SEQUENCE LISTING (180 sequences)

SEQ ID NO: 1 - DNA, 726 bp, Artificial Sequence
  Description of Artificial Sequence Synthetic polynucleotide
gctcttcgccgccgccactcctgctcgagcgcgcccgcgcgtgcgccgccagcgccttggccttttcgccgcgctcgtgcgcgtcgctgatgtccatcaccaggtccatgaggtctgccttgcgccggctgagccactgcttcgtccgggcggccaagaggagcatgagggaggactcctggtccagggtcctgacgtggtcgcggctctgggagcgggccagcatcatctggctctgccgcaccgaggccgcctccaactggtcctccagcagccgcagtcgccgccgaccctggcagaggaagacaggtgaggggggtatgaattgtacagaacaaccacgagccttgtctaggcagaatccctaccagtcatggctttacctggatgacggcctgcgaacagctgtccagcgaccctcgctgccgccgcttctcccgcacgcttctttccagcaccgtgatggcgcgagccagcgccgcacgctggcgctgcgcttcgccgatctgaggacagtcggggaactctgatcagtctaaacccccttgcgcgttagtgttgccatcctttgcagaccggtgagagccgacttgttgtgcgccaccccccacaccacctcctcccagaccaattctgtcacctttttggcgaaggcatcggcctcggcctgcagagaggacagcagtgcccagccgctgggggttggcggatgcacgctcaggtacc

SEQ ID NO: 2 - DNA, 749 bp, Artificial Sequence
  Description of Artificial Sequence Synthetic polynucleotide
gagctccttgttttccagaaggagttgctccttgagcctttcattctcagcctcgataacctccaaagccgctctaattgtggagggggttcgaatttaaaagcttggaatgttggttcgtgcgtctggaacaagcccagacttgttgctcactgggaaaaggaccatcagctccaaaaaacttgccgctcaaaccgcgtacctctgctttcgcgcaatctgccctgttgaaatcgccaccacattcatattgtgacgcttgagcagtctgtaattgcctcagaatgtggaatcatctgccccctgtgcgagcccatgccaggcatgtcgcgggcgaggacacccgccactcgtacagcagaccattatgctacctcacaatagttcataacagtgaccatatttctcgaagctccccaacgagcacctccatgctctgagtggccaccccccggccctggtgcttgcggagggcaggtcaaccggcatggggctaccgaaatccccgaccggatcccaccacccccgcgatgggaagaatctctccccgggatgtgggcccaccaccagcacaacctgctggcccaggcgagcgtcaaaccataccacacaaatatccttggcatcggccctgaattccttctgccgctctgctacccggtgcttctgtccgaagcaggggttgctagggatcgctccgagtccgcaaacccttgtcgcgtggcggggcttgttcgagcttgaagagc

SEQ ID NO: 3 - protein, 532 aa, Artificial Sequence
  Description of Artificial Sequence Synthetic polypeptide
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 4 - DNA, 1599 bp, Artificial Sequence
  Description of Artificial Sequence Synthetic polynucleotide
atgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtga

SEQ ID NO: 5 - DNA, 312 bp, Artificial Sequence
  Description of Artificial Sequence Synthetic polynucleotide
ctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaac

SEQ ID NO: 6 - DNA, 408 bp, Artificial Sequence
  Description of Artificial Sequence Synthetic polynucleotide
gcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctt

SEQ ID NO: 7 - DNA, 2333 bp, Artificial Sequence
  Description of Artificial Sequence Synthetic polynucleotide
ctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacggcgcgccatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcc

SEQ ID NO: 8 - DNA, 1065 bp, Prototheca moriformis
ggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagcc

SEQ ID NO: 9 - DNA, 120 bp, Artificial Sequence
  Description of Artificial Sequence Synthetic polynucleotide
actagtatggccaccgcatccactttctcggcgttcaatgcccgctgcggcgacctgcgtcgctcggcgggctccgggccccggcgcccagcgaggcccctccccgtgcgcgggcgcgcc

SEQ ID NO: 10 - DNA, 1236 bp, Cuphea wrightii
atggtggtggccgccgccgccagcagcgccttcttccccgtgcccgccccccgccccacccccaagcccggcaagttcggcaactggcccagcagcctgagccagcccttcaagcccaagagcaaccccaacggccgcttccaggtgaaggccaacgtgagcccccacgggcgcgcccccaaggccaacggcagcgccgtgagcctgaagtccggcagcctgaacaccctggaggacccccccagcagcccccccccccgcaccttcctgaaccagctgcccgactggagccgcctgcgcaccgccatcaccaccgtgttcgtggccgccgagaagcagttcacccgcctggaccgcaagagcaagcgccccgacatgctggtggactggttcggcagcgagaccatcgtgcaggacggcctggtgttccgcgagcgcttcagcatccgcagctacgagatcggcgccgaccgcaccgccagcatcgagaccctgatgaaccacctgcaggacaccagcctgaaccactgcaagagcgtgggcctgctgaacgacggcttcggccgcacccccgagatgtgcacccgcgacctgatctgggtgctgaccaagatgcagatcgtggtgaaccgctaccccacctggggcgacaccgtggagatcaacagctggttcagccagagcggcaagatcggcatgggccgcgagtggctgatcagcgactgcaacaccggcgagatcctggtgcgcgccaccagcgcctgggccatgatgaaccagaagacccgccgcttcagcaagctgccctgcgaggtgcgccaggagatcgccccccacttcgtggacgccccccccgtgatcgaggacaacgaccgcaagctgcacaagttcgacgtgaagaccggcgacagcatctgcaagggcctgacccccggctggaacgacttcgacgtgaaccagcacgtgagcaacgtgaagtacatcggctggattctggagagcatgcccaccgaggtgctggagacccaggagctgtgcagcctgaccctggagtaccgccgcgagtgcggccgcgagagcgtggtggagagcgtgaccagcatgaaccccagcaaggtgggcgaccgcagccagtaccagcacctgctgcgcctggaggacggcgccgacatcatgaagggccgcaccgagtggcgccccaagaacgccggcaccaaccgcgccatcagcacctga

SEQ ID NO: 11 - protein, 408 aa, Cuphea wrightii
MVVAAAASSAFFPVPAPRPTPKPGKFGNWPSSLSQPFKPKSNPNGRFQVKANVSPHPKANGSAVSLKSGSLNTLEDPPSSPPPRTFLNQLPDWSRLRTAITTVFVAAEKQFTRLDRKSKRPDMLVDWFGSETIVQDGLVFRERFSIRSYEIGADRTASIETLMNHLQDTSLNHCKSVGLLNDGFGRTPEMCTRDLIWVLTKMQIVVNRYPTWGDTVEINSWFSQSGKIGMGREWLISDCNTGEILVRATSAWAMMNQKTRRFSKLPCEVRQEIAPHFVDAPPVIEDNDRKLHKFDVKTGDSICKGLTPGWNDFDVNQHVSNVKYIGWILESMPTEVLETQELCSLTLEYRRECGRESVVESVTSMNPSKVGDRSQYQHLLRLEDGADIMKGRTEWRPKNAGTNRAIST

SEQ ID NO: 12 - DNA, 933 bp, Artificial Sequence
  Description of Artificial Sequence Synthetic polynucleotide
atggacgcctccggcgcctcctccttcctgcgcggccgctgcctggagtcctgcttcaaggcctccttcggctacgtaatgtcccagcccaaggacgccgccggccagccctcccgccgccccgccgacgccgacgacttcgtggacgacgaccgctggatcaccgtgatcctgtccgtggtgcgcatcgccgcctgcttcctgtccatgatggtgaccaccatcgtgtggaacatgatcatgctgatcctgctgccctggccctacgcccgcatccgccagggcaacctgtacggccacgtgaccggccgcatgctgatgtggattctgggcaaccccatcaccatcgagggctccgagttctccaacacccgcgccatctacatctgcaaccacgcctccctggtggacatcttcctgatcatgtggctgatccccaagggcaccgtgaccatcgccaagaaggagatcatctggtatcccctgttcggccagctgtacgtgctggccaaccaccagcgcatcgaccgctccaacccctccgccgccatcgagtccatcaaggaggtggcccgcgccgtggtgaagaagaacctgtccctgatcatcttccccgagggcacccgctccaagaccggccgcctgctgcccttcaagaagggcttcatccacatcgccctccagacccgcctgcccatcgtgccgatggtgctgaccggcacccacctggcctggcgcaagaactccctgcgcgtgcgccccgcccccatcaccgtgaagtacttctcccccatcaagaccgacgactgggaggaggagaagatcaaccactacgtggagatgatccacgccctgtacgtggaccacctgcccgagtcccagaagcccctggtgtccaagggccgcgacgcctccggccgctccaactcctga

SEQ ID NO: 13 - DNA, 563 bp, Artificial Sequence
  Description of Artificial Sequence Synthetic polynucleotide
gctcttcgctaacggaggtctgtcaccaaatggaccccgtctattgcgggaaaccacggcgatggcacgtttcaaaacttgatgaaatacaatattcagtatgtcgcgggcggcgacggcggggagctgatgtcgcgctgggtattgcttaatcgccagcttcgcccccgtcttggcgcgaggcgtgaacaagccgaccgatgtgcacgagcaaatcctgacactagaagggctgactcgcccggcacggctgaattacacaggcttgcaaaaataccagaatttgcacgcaccgtattcgcggtattttgttggacagtgaatagcgatgcggcaatggcttgtggcgttagaaggtgcgacgaaggtggtgccaccactgtgccagccagtcctggcggctcccagggccccgatcaagagccaggacatccaaactacccacagcatcaacgccccggcctatactcgaaccccacttgcactctgcaatggtatgggaaccacggggcagtcttgtgtgggtcgcgcctatcgcggtcggcgaagaccgggaaggtacc

SEQ ID NO: 14 - DNA, 465 bp, Artificial Sequence
  Description of Artificial Sequence Synthetic polynucleotide
gagctcagcggcgacggtcctgctaccgtacgacgttgggcacgcccatgaaagtttgtataccgagcttgttgagcgaactgcaagcgcggctcaaggatacttgaactcctggattgatatcggtccaataatggatggaaaatccgaacctcgtgcaagaactgagcaaacctcgttacatggatgcacagtcgccagtccaatgaacattgaagtgagcgaactgttcgcttcggtggcagtactactcaaagaatgagctgctgttaaaaatgcactctcgttctctcaagtgagtggcagatgagtgctcacgccttgcacttcgctgcccgtgtcatgccctgcgccccaaaatttgaaaaaagggatgagattattgggcaatggacgacgtcgtcgctccgggagtcaggaccggcggaaaataagaggcaacacactccgcttcttagctcttcc

SEQ ID NO: 15 - DNA, 1533 bp, Artificial Sequence
  Description of Artificial Sequence Synthetic polynucleotide
ctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgatcgagcaggacggcctccacgccggctcccccgccgcctgggtggagcgcctgttcggctacgactgggcccagcagaccatcggctgctccgacgccgccgtgttccgcctgtccgcccagggccgccccgtgctgttcgtgaagaccgacctgtccggcgccctgaacgagctgcaggacgaggccgcccgcctgtcctggctggccaccaccggcgtgccctgcgccgccgtgctggacgtggtgaccgaggccggccgcgactggctgctgctgggcgaggtgcccggccaggacctgctgtcctcccacctggcccccgccgagaaggtgtccatcatggccgacgccatgcgccgcctgcacaccctggaccccgccacctgccccttcgaccaccaggccaagcaccgcatcgagcgcgcccgcacccgcatggaggccggcctggtggaccaggacgacctggacgaggagcaccagggcctggcccccgccgagctgttcgcccgcctgaaggcccgcatgcccgacggcgaggacctggtggtgacccacggcgacgcctgcctgcccaacatcatggtggagaacggccgcttctccggcttcatcgactgcggccgcctgggcgtggccgaccgctaccaggacatcgccctggccacccgcgacatcgccgaggagctgggcggcgagtgggccgaccgcttcctggtgctgtacggcatcgccgcccccgactcccagcgcatcgccttctaccgcctgctggacgagttcttctgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcc

SEQ ID NO: 16 - protein, 310 aa, Cocos nucifera
MDASGASSFLRGRCLESCFKASFGYVMSQPKDAAGQPSRRPADADDFVDDDRWITVILSVVRIAACFLSMMVTTIVWNMIMLILLPWPYARIRQGNLYGHVTGRMLMWILGNPITIEGSEFSNTRAIYICNHASLVDIFLIMWLIPKGTVTIAKKEIIWYPLFGQLYVLANHQRIDRSNPSAAIESIKEVARAVVKKNLSLIIFPEGTRSKTGRLLPFKKGFIHIALQTRLPIVPMVLTGTHLAWRKNSLRVRPAPITVKYFSPIKTDDWEEEKINHYVEMIHALYVDHLPESQKPLVSKGRDASGRSNS

SEQ ID NO: 17 - DNA, 1476 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtga

SEQ ID NO: 18 - protein, 491 aa, Artificial Sequence
  Description of Artificial Sequence Synthetic polypeptide
MATASTFSAFNARCGDLRRSAGSGPRRPARPLPVRGRAAAAADANPARPERRVVITGQGVVTSLGQTIEQFYSSLLEGVSGISQIQKFDTTGYTTTIAGEIKSLQLDPYVPKRWAKRVDDVIKYVYIAGKQALESAGLPIEAAGLAGAGLDPALCGVLIGTAMAGMTSFAAGVEALTRGGVRKMNPFCIPFSISNMGGAMLAMDIGFMGPNYSISTACATGNYCILGAADHIRRGDANVMLAGGADAAIIPSGIGGFIACKALSKRNDEPERASRPWDADRDGFVMGEGAGVLVLEELEHAKRRGATILAELVGGAATSDAHHMTEPDPQGRGVRLCLERALERARLAPERVGYVNAHGTSTPAGDVAEYRAIRAVIPQDSLRINSTKSMIGHLLGGAGAVEAVAAIQALRTGWLHPNLNLENPAPGVDPVVLVGPRKERAEDLDVVLSNSFGFGGHNSCVIFRKYDEMDYKDHDGDYKDHDIDYKDDDDK

SEQ ID NO: 19 - DNA, 1590 bp, Artificial Sequence
  Description of Artificial Sequence Synthetic polynucleotide
atggactcccgcgcccagaaccgcgacggcggcgaggacgtgaagcaggagctgctgtccgccggcgacgacggcaaggtgccctgccccaccgtggccatcggcatccgccagcgcctgcccgacttcctgcagtccgtgaacatgaagtacgtgaagctgggctaccactacctgatcacccacgccatgttcctgctgaccctgcccgccttcttcctggtggccgccgagatcggccgcctgggccacgagcgcatctaccgcgagctgtggacccacctgcacctgaacctggtgtccatcatggcctgctcctccgccctggtggccggcgccaccctgtacttcatgtcccgcccccgccccgtgtacctggtggagttcgcctgctaccgccccgacgagcgcctgaaggtgtccaaggacttcttcctggacatgtcccgccgcaccggcctgttctcctcctcctccatggacttccagaccaagatcacccagcgctccggcctgggcgacgagacctacctgccccccgccatcctggcctccccccccaacccctgcatgcgcgaggcccgcgaggaggccgccatggtgatgttcggcgccctggacgagctgttcgagcagaccggcgtgaagcccaaggagatcggcgtgctggtggtgaactgctccctgttcaaccccaccccctccatgtccgccatgatcgtgaaccactaccacatgcgcggcaacatcaagtccctgaacctgggcggcatgggctgctccgccggcctgatctccatcgacctggcccgcgacctgctgcaggtgcacggcaacacctacgccgtggtggtgtccaccgagaacatcaccctgaactggtacttcggcgacgaccgctccaagctgatgtccaactgcatcttccgcatgggcggcgccgccgtgctgctgtccaacaagcgccgcgagcgccgccgcgccaagtacgagctgctgcacaccgtgcgcacccacaagggcgccgacgacaagtgcttccgctgcgtgtaccaggaggaggactccaccggctccctgggcgtgtccctgtcccgcgagctgatggccgtggccggcaacgccctgaaggccaacatcaccaccctgggccccctggtgctgcccctgtccgagcagatcctgttcttcgcctccctggtggcccgcaagttcctgaacatgaagatgaagccctacatccccgacttcaagctggccttcgagcacttctgcatccacgccggcggccgcgccgtgctggacgagctggagaagaacctggacctgaccgagtggcacatggagccctcccgcatgaccctgtaccgcttcggcaacacctcctcctcctccctgtggtacgagctggcctacaccgaggcccagggccgcgtgaagcgcggcgaccgcctgtggcagatcgccttcggctccggcttcaagtgcaactccgccgtgtggcgcgcgctgcgcaccgtgaagccccccgtgaacaacgcctggtccgacgtgatcgaccgcttccccgtgaagctgccccagttctga

SEQ ID NO: 20 - protein, 529 aa, Artificial Sequence
  Description of Artificial Sequence Synthetic polypeptide
MDSRAQNRDGGEDVKQELLSAGDDGKVPCPTVAIGIRQRLPDFLQSVNMKYVKLGYHYLITHAMFLLTLPAFFLVAAEIGRLGHERIYRELWTHLHLNLVSIMACSSALVAGATLYFMSRPRPVYLVEFACYRPDERLKVSKDFFLDMSRRTGLFSSSSMDFQTKITQRSGLGDETYLPPAILASPPNPCMREAREEAAMVMFGALDELFEQTGVKPKEIGVLVVNCSLFNPTPSMSAMIVNHYHMRGNIKSLNLGGMGCSAGLISIDLARDLLQVHGNTYAVVVSTENITLNWYFGDDRSKLMSNCIFRMGGAAVLLSNKRRERRRAKYELLHTVRTHKGADDKCFRCVYQEEDSTGSLGVSLSRELMAVAGNALKANITTLGPLVLPLSEQILFFASLVARKFLNMKMKPYIPDFKLAFEHFCIHAGGRAVLDELEKNLDLTEWHMEPSRMTLYRFGNTSSSSLWYELAYTEAQGRVKRGDRLWQIAFGSGFKCNSAVWRALRTVKPPVNNAWSDVIDRFPVKLPQF

SEQ ID NO: 21 - DNA, 906 bp, Trypanosoma brucei
atgctgatgaacttcggcggctcctacgacgcctacatcaacaacttccagggcaccttcctggccgagtggatgctggaccacccctccgtgccctacatcgccggcgtgatgtacctgatcctggtgctgtacgtgcccaagtccatcatggcctcccagccccccctgaacctgcgcgccgccaacatcgtgtggaacctgttcctgaccctgttctccatgtgcggcgcctactacaccgtgccctacctggtgaaggccttcatgaaccccgagatcgtgatggccgcctccggcatcaagctggacgccaacacctcccccatcatcacccactccggcttctacaccaccacctgcgccctggccgactccttctacttcaacggcgacgtgggcttctgggtggccctgttcgccctgtccaagatccccgagatgatcgacaccgccttcctggtgttccagaagaagcccgtgatcttcctgcactggtaccaccacctgaccgtgatgctgttctgctggttcgcctacgtgcagaagatctcctccggcctgtggttcgcctccatgaactactccgtgcactccatcatgtacctgtactacttcgtgtgcgcctgcggccaccgccgcctggtgcgccccttcgcccccatcatcaccttcgtgcagatcttccagatggtggtgggcaccatcgtggtgtgctacacctacaccgtgaagcacgtgctgggccgctcctgcaccgtgaccgacttctccctgcacaccggcctggtgatgtacgtgtcctacctgctgctgttctcccagctgttctaccgctcctacctgtccccccgcgacaaggcctccatcccccacgtggccgccgagatcaagaagaaggagtga

SEQ ID NO: 22 - protein, 322 aa, Trypanosoma brucei
MYPTHRDLILNNYSDIYRSPTCHYHTWHTLIHTPINELLFPNLPRECDFGYDIPYFRGQIDVFDGWSMIHFTSSNWCIPITVCLCYIMMIAGLKKYMGPRDGGRAPIQAKNYIIAWNLFLSFFSFAGVYYTVPYHLFDPENGLFAQGFYSTVCNNGAYYGNGNVGFFVWLFIYSKIFELVDTFFLLIRKNPVIFLHWYHHLTVLLYCWHAYSVRIGTGIWFATMNYSVHSVMYLYFAMTQYGPSTKKFAKKFSKFITTIQILQMVVGIIVTFAAMLYVTFDVPCYTSLANSVLGLMMYASYFVLFVQLYVSHYVSPKHVKQE

SEQ ID NO: 23 - DNA, 933 bp, Artificial Sequence
  Description of Artificial Sequence Synthetic polynucleotide
atggtgtccgactggaagaacttctgcctggagaaggcctcccgcttccgccccaccatcgaccgccccttcttcaacatctacctgtgggactacttcaaccgcgccgtgggctgggccaccgccggccgcttccagcccaaggacttcgagttcaccgtgggcaagcagcccctgtccgagccccgccccgtgctgctgttcatcgccatgtactacgtggtgatcttcggcggccgctccctggtgaagtcctgcaagcccctgaagctgcgcttcatctcccaggtgcacaacctgatgctgacctccgtgtccttcctgtggctgatcctgatggtggagcagatgctgcccatcgtgtaccgccacggcctgtacttcgccgtgtgcaacgtggagtcctggacccagcccatggagaccctgtactacctgaactacatgaccaagttcgtggagttcgccgacaccgtgctgatggtgctgaagcaccgcaagctgaccttcctgcacacctaccaccacggcgccaccgccctgctgtgctacaaccagctggtgggctacaccgccgtgacctgggtgcccgtgaccctgaacctggccgtgcacgtgctgatgtactggtactacttcctgtccgcctccggcatccgcgtgtggtggaaggcctgggtgacccgcctgcagatcgtgcagttcatgctggacctgatcgtggtgtactacgtgctgtaccagaagatcgtggccgcctacttcaagaacgcctgcaccccccagtgcgaggactgcctgggctccatgaccgccatcgccgccggcgccgccatcctgacctcctacctgttcctgttcatctccttctacatcgaggtgtacaagcgcggctccgcctccggcaagaagaagatcaacaagaacaactga

SEQ ID NO: 24 - protein, 310 aa, Artificial Sequence
  Description of Artificial Sequence Synthetic polypeptide
MVSDWKNFCLEKASRFRPTIDRPFFNIYLWDYFNRAVGWATAGRFQPKDFEFTVGKQPLSEPRPVLLFIAMYYVVIFGGRSLVKSCKPLKLRFISQVHNLMLTSVSFLWLILMVEQMLPIVYRHGLYFAVCNVESWTQPMETLYYLNYMTKFVEFADTVLMVLKHRKLTFLHTYHHGATALLCYNQLVGYTAVTWVPVTLNLAVHVLMYWYYFLSASGIRVWWKAWVTRLQIVQFMLDLIVVYYVLYQKIVAAYFKNACTPQCEDCLGSMTAIAAGAAILTSYLFLFISFYIEVYKRGSASGKKKINKNN

SEQ ID NO: 25 - DNA, 573 bp, Prototheca moriformis
tgttgaagaatgagccggcgacttaaaataaatggcaggctaagagaattaataactcgaaacctaagcgaaagcaagtcttaatagggcgctaatttaacaaaacattaaataaaatctaaagtcatttattttagacccgaacctgagtgatctaaccatggtcaggatgaaacttgggtgacaccaagtggaagtccgaaccgaccgatgttgaaaaatcggcggatgaactgtggttagtggtgaaataccagtcgaactcagagctagctggttctccccgaaatgcgttgaggcgcagcaatatatctcgtctatctaggggtaaagcactgtttcggtgcgggctatgaaaatggtaccaaatcgtggcaaactctgaatactagaaatgacgatatattagtgagactatgggggataagctccatagtcgagagggaaacagcccagaccaccagttaaggccccaaaatgataatgaagtggtaaaggaggtgaaaatgcaaatacaaccaggaggttggcttagaagcagccatcctttaaagagtgcgtaatagctcactg

SEQ ID NO: 26 - protein, 384 aa, Cuphea wrightii
MAIAAAAVIFLFGLIFFASGLIINLFQALCFVLIRPLSKNAYRRINRVFAELLLSELLCLFDWWAGAKLKLFTDPETFRLMGKEHALVIINHMTELDWMVGWVMGQHFGCLGSIISVAKKSTKFLPVLGWSMWFSEYLYLERSWAKDKSTLKSHIERLIDYPLPFWLVIFVEGTRFTRTKLLAAQQYAVSSGLPVPRNVLIPRTKGFVSCVSHMRSFVPAVYDVTVAFPKTSPPPTLLNLFEGQSIMLHVHIKRHAMKDLPESDDAVAEWCRDKFVEKDALLDKHNAEDTFSGQEVCHSGSRQLKSLLVVISWVVVTTFGALKFLQWSSWKGKAFSAIGLGIVTLLMHVLILSSQAERSNPAEVAQAKLKTGLSISKKVTDKEN

SEQ ID NO: 27 - protein, 387 aa, Cuphea wrightii
MAIAAAAVIVPLSLLFFVSGLIVNLVQAVCFVLIRPLSKNTYRRINRVVAELLWLELVWLIDWWAGVKIKVFTDHETFHLMGKEHALVICNHKSDIDWLVGWVLGQRSGCLGSTLAVMKKSSKFLPVLGWSMWFSEYLFLERSWAKDEITLKSGLNRLKDYPLPFWLALFVEGTRFTRAKLLAAQQYAASSGLPVPRNVLIPRTKGFVSSVSHMRSFVPAIYDVTVAIPKTSPPPTLIRMFKGQSSVLHVHLKRHLMKDLPESDDAVAQWCRDIFVEKDALLDKHNAEDTFSGQELQETGRPIKSLLVVISWAVLEVFGAVKFLQWSSLLSSWKGLAFSGIGLGVITLLMHILILFSQSERSTPAKVAPAKPKNEGESSKTEMEKEK

SEQ ID NO: 28 - protein, 327 aa, Cuphea wrightii
MEIPPHCLCSPSPAPSQLYYKKKKHAILQTQTPYRYRVSPTCFAPPRLRKQHPYPLPVLCYPKLLHFSQPRYPLVRSHLAEAGVAYRPGYELLGKIRGVCFYAVTAAVALLLFQCMLLLHPFVLLFDPFPRKAHHTIAKLWSICSVSLFYKIHIKGLENLPPPHSPAVYVSNHQSFLDIYTLLTLGRTFKFISKTEIFLYPIIGWAMYMLGTIPLKRLDSRSQLDTLKRCMDLIKKGASVFFFPEGTRSKDGKLGAFKKGAFSIAAKSKVPVVPITLIGTGKIMPPGSELTVNPGTVQVIIHKPIEGSDAEAMCNEARATISHSLDD

SEQ ID NO: 29 - DNA, 984 bp, Cuphea wrightii
atggagatcccgcctcactgtctctgttcgccttcgcctgcgccttcgcaattgtattacaagaagaagaagcatgccattctccaaactcaaactccctatagatatagagtttccccgacatgctttgcccccccccgattgaggaagcagcatccttaccctctccctgtcctctgctatccaaaactcctccacttcagccagcctaggtaccctctggttagatctcatttggctgaagctggtgttgcttatcgtccaggatacgaattattaggaaaaataaggggagtgtgtttctatgctgtcactgctgccgttgccttgcttctatttcagtgcatgctcctcctccatccctttgtgctcctcttcgatccatttccaagaaaggctcaccataccatcgccaaactctggtctatctgctctgtttctcttttttacaagattcacatcaagggtttggaaaatcttcccccaccccactctcctgccgtctatgtctctaatcatcagagttttctcgacatctatactctcctcactctcggtagaaccttcaagttcatcagcaagactgagatctttctctatccaattatcggttgggccatgtatatgttgggtaccattcctctcaagcggttggacagcagaagccaattggacactcttaagcgatgtatggatctcatcaagaagggagcatccgtctttttcttcccagagggaacacgaagtaaagatgggaaactgggtgctttcaagaaaggtgcattcagcatcgcagcaaaaagcaaggttcctgttgtgccgatcacccttattggaactggcaagattatgccacctgggagcgaacttactgtcaatccaggaactgtgcaagtaatcatacataaacctatcgaaggaagtgatgcagaagcaatgtgcaatgaagctagagccacgatttctcactcacttgatgattaa

SEQ ID NO: 30 - DNA, 1155 bp, Cuphea wrightii
atggcgattgcagcggcagctgtcatcttcctcttcggccttatcttcttcgcctccggcctcataatcaatctcttccaggcgctttgctttgtccttattcggcctctttcgaaaaacgcctacmggagaataaacagagtttttgcagaattgttgttgtcggagcttttatgcctattcgattggtgggctggtgctaagctcaaattatttaccgaccctgaaacctttcgccttatgggcaaggaacatgctcttgtcataattaatcacatgactgaacttgactggatggttggatgggttatgggtcagcattttggttgccttgggagcataatatctgttgcgaagaaatcaacaaaatttcttccggtattggggtggtcaatgtggttttcagagtacctatatcttgagagaagctgggccaaggataaaagtacattaaagtcacatatcgagaggctgatagactaccccctgcccttctggttggtaatttttgtggaaggaactcggtttactcggacaaaactcttggcagcccagcagtatgctgtctcatctgggctaccagtgccgagaaatgttttgatcccacgtactaagggttttgtttcatgtgtaagtcacatgcgatcatttgttccagcagtatatgatgtcacagtggcattccctaagacttcacctccaccaacgttgctaaatcttttcgagggtcagtccataatgcttcacgttcacatcaagcgacatgcaatgaaagatttaccagaatccgatgatgcagtagcagagtggtgtagagacaaatttgtggaaaaggatgctttgttggacaagcataatgctgaggacactttcagtggtcaagaagtttgtcatagcggcagccgccagttaaagtctcttctggtggtaatatcttgggtggttgtaacaacatttggggctctaaagttccttcagtggtcatcatggaaggggaaagcattttcagctatcgggctgggcatcgtcactctacttatgcacgtattgattctatcctcacaagcagagcggtctaaccctgcggaggtggcacaggcaaagctaaagaccgggttgtcgatctcaaagaaggtaacggacaaggaaaactag

SEQ ID NO: 31 - DNA, 1164 bp, Cuphea wrightii
atggcgattgctgcggcagctgtcatcgtcccgctcagcctcctcttcttcgtctccggcctcatcgtcaatctcgtacaggcagtttgctttgtactgattaggcctctgtcgaaaaacacttacagaagaataaacagagtggttgcagaattgttgtggttggagttggtatggctgattgattggtgggctggtgtcaagataaaagtattcacggatcatgaaacctttcaccttatgggcaaagaacatgctcttgtcatttgtaatcacaagagtgacatagactggctggttgggtgggttctgggacagcggtcaggttgccttggaagcacattagctgttatgaagaaatcatcaaagtttctcccggtattagggtggtcaatgtggttctcagagtatctattccttgaaagaagctgggccaaggatgaaattacattaaagtcaggtttgaataggctgaaagactatcccttacccttctggttggcactttttgtggaaggaactcggttcactcgagcaaaactcttggcagcccagcagtatgctgcctcttcggggctacctgtgccgagaaatgttctgatcccgcgtactaagggttttgtttcttctgtgagtcacatgcgatcatttgttccagccatatatgatgttacagtggcaatcccaaagacgtcacctccaccaacattgataagaatgttcaagggacagtcctcagtgcttcacgtccacctcaagcgacacctaatgaaagatttacctgaatcagatgatgctgttgctcagtggtgcagagatatattcgtcgagaaggatgctttgttggataagcataatgctgaggacactttcagtggccaagaacttcaagaaactggccgcccaataaagtctcttctggttgtaatctcttgggcggtgttggaggtatttggagctgtgaagtttcttcaatggtcatcgctgttatcatcatggaagggacttgcattttcgggaataggactgggtgtcatcacgctactcatgcacatactgattttattctcacaatccgagcggtctacccctgcaaaagtggcaccagcaaagccaaagaatgagggagagtcctccaagacggaaatggaaaaggaaaagtag

SEQ ID NO: 32 - DNA, 984 bp, Artificial Sequence
  Description of Artificial Sequence Synthetic polynucleotide
atggagatccccccccactgcctgtgctccccctcccccgccccctcccagctgtactacaagaagaagaagcacgccatcctgcagacccagaccccctaccgctaccgcgtgtcccccacctgcttcgcccccccccgcctgcgcaagcagcacccctaccccctgcccgtgctgtgctaccccaagctgctgcacttctcccagccccgctaccccctggtgcgctcccacctggccgaggccggcgtggcctaccgccccggctacgagctgctgggcaagatccgcggcgtgtgcttctacgccgtgaccgccgccgtggccctgctgctgttccagtgcatgctgctgctgcaccccttcgtgctgctgttcgaccccttcccccgcaaggcccaccacaccatcgccaagctgtggtccatctgctccgtgtccctgttctacaagatccacatcaagggcctggagaacctgccccccccccactcccccgccgtgtacgtgtccaaccaccagtccttcctggacatctacaccctgctgaccctgggccgcaccttcaagttcatctccaagaccgagatcttcctgtaccccatcatcggctgggccatgtacatgctgggcaccatccccctgaagcgcctggactcccgctcccagctggacaccctgaagcgctgcatggacctgatcaagaagggcgcctccgtgttcttcttccccgagggcacccgctccaaggacggcaagctgggcgccttcaagaagggcgccttctccatcgccgccaagtccaaggtgcccgtggtgcccatcaccctgatcggcaccggcaagatcatgccccccggctccgagctgaccgtgaaccccggcaccgtgcaggtgatcatccacaagcccatcgagggctccgacgccgaggccatgtgcaacgaggcccgcgccaccatctcccactccctggacgactga

SEQ ID NO: 33 - DNA, 1155 bp, Artificial Sequence
  Description of Artificial Sequence Synthetic polynucleotide
atggcgatcgcggccgcggcggtgatcttcctgttcggcctgatcttcttcgcctccggcctgatcatcaacctgttccaggcgctgtgcttcgtcctgatccgccccctgtccaagaacgcctaccgccgcatcaaccgcgtgttcgcggagctgctgctgtccgagctgctgtgcctgttcgactggtgggcgggcgcgaagctgaagctgttcaccgaccccgagacgttccgcctgatgggcaaggagcacgccctggtcatcatcaaccacatgaccgagctggactggatggtgggctgggtgatgggccagcacttcggctgcctgggctccatcatctccgtcgccaagaagtccacgaagttcctgcccgtgctgggctggtccatgtggttctccgagtacctgtacctggagcgctcctgggccaaggacaagtccaccctgaagtcccacatcgagcgcctgatcgactaccccctgcccttctggctggtcatcttcgtcgagggcacccgcttcacgcgcacgaagctgctggcggcccagcagtacgcggtctcctccggcctgcccgtcccccgcaacgtcctgatcccccgcacgaagggcttcgtctcctgcgtgtcccacatgcgctccttcgtccccgcggtgtacgacgtcacggtggcgttccccaagacgtcccccccccccacgctgctgaacctgttcgagggccagtccatcatgctgcacgtgcacatcaagcgccacgccatgaaggacctgcccgagtccgacgacgccgtcgcggagtggtgccgcgacaagttcgtcgagaaggacgccctgctggacaagcacaacgcggaggacacgttctccggccaggaggtgtgccactccggctcccgccagctgaagtccctgctggtcgtgatctcctgggtcgtggtgacgacgttcggcgccctgaagttcctgcagtggtcctcctggaagggcaaggcgttctccgccatcggcctgggcatcgtcaccctgctgatgcacgtgctgatcctgtcctcccaggccgagcgctccaaccccgccgaggtggcccaggccaagctgaagaccggcctgtccatctccaagaaggtgacggacaaggagaactga

SEQ ID NO: 34 - DNA, 1164 bp, Artificial Sequence
  Description of Artificial Sequence Synthetic polynucleotide
atggccatcgcggcggccgcggtgatcgtgcccctgtccctgctgttcttcgtgtccggcctgatcgtcaacctggtgcaggccgtctgcttcgtcctgatccgccccctgtccaagaacacgtaccgccgcatcaaccgcgtggtcgcggagctgctgtggctggagctggtgtggctgatcgactggtgggcgggcgtgaagatcaaggtcttcacggaccacgagacgttccacctgatgggcaaggagcacgccctggtcatctgcaaccacaagtccgacatcgactggctggtcggctgggtcctgggccagcgctccggctgcctgggctccaccctggcggtcatgaagaagtcctccaagttcctgcccgtcctgggctggtccatgtggttctccgagtacctgttcctggagcgctcctgggccaaggacgagatcacgctgaagtccggcctgaaccgcctgaaggactaccccctgcccttctggctggcgctgttcgtggagggcacgcgcttcacccgcgcgaagctgctggcggcgcagcagtacgccgcgtcctccggcctgcccgtgccccgcaacgtgctgatcccccgcacgaagggcttcgtgtcctccgtgtcccacatgcgctccttcgtgcccgcgatctacgacgtcaccgtggccatccccaagacgtcccccccccccacgctgatccgcatgttcaagggccagtcctccgtgctgcacgtgcacctgaagcgccacctgatgaaggacctgcccgagtccgacgacgccgtcgcgcagtggtgccgcgacatcttcgtggagaaggacgcgctgctggacaagcacaacgccgaggacaccttctccggccaggagctgcaggagaccggccgccccatcaagtccctgctggtcgtcatctcctgggccgtcctggaggtgttcggcgccgtcaagttcctgcagtggtcctccctgctgtcctcctggaagggcctggcgttctccggcatcggcctgggcgtgatcaccctgctgatgcacatcctgatcctgttctcccagtccgagcgctccacccccgccaaggtggcccccgcgaagcccaagaacgagggcgagtcctccaagaccgagatggagaaggagaagtga

SEQ ID NO: 35 - DNA, 7041 bp, Artificial Sequence
  Description of Artificial Sequence Synthetic polynucleotide
gctcttcgccgccgccactcctgctcgagcgcgcccgcgcgtgcgccgccagcgccttggccttttcgccgcgctcgtgcgcgtcgctgatgtccatcaccaggtccatgaggtctgccttgcgccggctgagccactgcttcgtccgggcggccaagaggagcatgagggaggactcctggtccagggtcctgacgtggtcgcggctctgggagcgggccagcatcatctggctctgccgcaccgaggccgcctccaactggtcctccagcagccgcagtcgccgccgaccctggcagaggaagacaggtgaggggggtatgaattgtacagaacaaccacgagccttgtctaggcagaatccctaccagtcatggctttacctggatgacggcctgcgaacagctgtccagcgaccctcgctgccgccgcttctcccgcacgcttctttccagcaccgtgatggcgcgagccagcgccgcacgctggcgctgcgcttcgccgatctgaggacagtcggggaactctgatcagtctaaacccccttgcgcgttagtgttgccatcctttgcagaccggtgagagccgacttgttgtgcgccaccccccacaccacctcctcccagaccaattctgtcacctttttggcgaaggcatcggcctcggcctgcagagaggacagcagtgcccagccgctgggggttggcggatgcacgctcaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgaattcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagaactcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccactagtatgacctccatcaacgtgaagctgctgtaccactacgtgatcaccaacctgttcaacctgtgcttcttccccctgaccgccatcgtggccggcaaggcctcccgcctgaccatcgacgacctgcaccacctgtactactcctacctgcagcacaacgtgatcaccatcgcccccctgttcgccttcaccgtgttcggctccatcctgtacatcgtgacccgccccaagcccgtgtacctggtggagtactcctgctacctgccccccacccagtgccgctcctccatctccaaggtgatggacatcttctaccaggtgcgcaaggccgaccccttccgcaacggcacctgcgacgactcctcctggctggacttcctgcgcaagatccaggagcgctccggcctgggcgacgagacccacggccccgagggcctgctgcaggtgcccccccgcaagaccttcgccgccgcccgcgaggagaccgagcaggtgatcgtgggcgccctgaagaacctgttcgagaacaccaaggtgaaccccaaggacatcggcatcctggtggtgaactcctccatgttcaaccccaccccctccctgtccgccatggtggtgaacaccttcaagctgcgctccaacgtgcgctccttcaacctgggcggcatgggctgctccgccggcgtgatcgccatcgacctggccaaggacctgctgcacgtgcacaagaacacctacgccctggtggtgtccaccgagaacatcacctacaacatctacgccggcgacaaccgctccatgatggtgtccaactgcctgttccgcgtgggcggcgccgccatcctgctgtccaacaagccccgcgaccgccgccgctccaagtacgagctggtgcacaccgtgcgcacccacaccggcgccgacgacaagtccttccgctgcgtgcagcagggcgacgacgagaacggcaagaccggcgtgtccctgtccaaggacatcaccgaggtggccggccgcaccgtgaagaagaacatcgccaccctgggccccctgatcctgcccctgtccgagaagctgctgttcttcgtgaccttcatggccaagaagctgttcaaggacaaggtgaagcactactacgtgcccgacttcaagctggccatcgaccacttctgcatccacgccggcggccgcgccgtgatcgacgtgctggagaagaacctgggcctggcccccatcgacgtggaggcctcccgctccaccctgcaccgcttcggcaacacctcctcctcctccatctggtacgagctggcctacatcgaggccaagggccgcatgaagaagggcaacaaggtgtggcagatcgccctgggctccggcttcaagtgcaactccgccgtgtgggtggccctgtccaacgtgaaggcctccaccaactccccctgggagcactgcatcgaccgctaccccgtgaagatcgactccgactccgccaagtccgagacccgcgcccagaacggccgctcctgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcttgttttccagaaggagttgctccttgagcctttcattctcagcctcgataacctccaaagccgctctaattgtggagggggttcgaatttaaaagcttggaatgttggttcgtgcgtctggaacaagcccagacttgttgctcactgggaaaaggaccatcagctccaaaaaacttgccgctcaaaccgcgtacctctgctttcgcgcaatctgccctgttgaaatcgccaccacattcatattgtgacgcttgagcagtctgtaattgcctcagaatgtggaatcatctgccccctgtgcgagcccatgccaggcatgtcgcgggcgaggacacccgccactcgtacagcagaccattatgctacctcacaatagttcataacagtgaccatatttctcgaagctccccaacgagcacctccatgctctgagtggccaccccccggccctggtgcttgcggagggcaggtcaaccggcatggggctaccgaaatccccgaccggatcccaccacccccgcgatgggaagaatctctccccgggatgtgggcccaccaccagcacaacctgctggcccaggcgagcgtcaaaccataccacacaaatatccttggcatcggccctgaattccttctgccgctctgctacccggtgcttctgtccgaagcaggggttgctagggatcgctccgagtccgcaaacccttgtcgcgtggcggggcttgttcgagcttgaagagc

SEQ ID NO: 36 - DNA, 1530 bp, Artificial Sequence
  Description of Artificial Sequence Synthetic polynucleotide
actagtatgacctccatcaacgtgaagctgctgtaccactacgtgatcaccaacttcttcaacctgtgcttcttccccctgaccgccatcctggccggcaaggcctcccgcctgaccaccaacgacctgcaccacttctactcctacctgcagcacaacctgatcaccctgaccctgctgttcgccttcaccgtgttcggctccgtgctgtacttcgtgacccgccccaagcccgtgtacctggtggactactcctgctacctgcccccccagcacctgtccgccggcatctccaagaccatggagatcttctaccagatccgcaagtccgaccccctgcgcaacgtggccctggacgactcctcctccctggacttcctgcgcaagatccaggagcgctccggcctgggcgacgagacctacggccccgagggcctgttcgagatccccccccgcaagaacctggcctccgcccgcgaggagaccgagcaggtgatcaacggcgccctgaagaacctgttcgagaacaccaaggtgaaccccaaggagatcggcatcctggtggtgaactcctccatgttcaaccccaccccctccctgtccgccatggtggtgaacaccttcaagctgcgctccaacatcaagtccttcaacctgggcggcatgggctgctccgccggcgtgatcgccatcgacctggccaaggacctgctgcacgtgcacaagaacacctacgccctggtggtgtccaccgagaacatcacccagaacatctacaccggcgacaaccgctccatgatggtgtccaactgcctgttccgcgtgggcggcgccgccatcctgctgtccaacaagcccggcgaccgccgccgctccaagtaccgcctggcccacaccgtgcgcacccacaccggcgccgacgacaagtccttcggctgcgtgcgccaggaggaggacgactccggcaagaccggcgtgtccctgtccaaggacatcaccggcgtggccggcatcaccgtgcagaagaacatcaccaccctgggccccctggtgctgcccctgtccgagaagatcctgttcgtggtgaccttcgtggccaagaagctgctgaaggacaagatcaagcactactacgtgcccgacttcaagctggccgtggaccacttctgcatccacgccggcggccgcgccgtgatcgacgtgctggagaagaacctgggcctgtcccccatcgacgtggaggcctcccgctccaccctgcaccgcttcggcaacacctcctcctcctccatctggtacgagctggcctacatcgaggccaagggccgcatgaagaagggcaacaaggcctggcagatcgccgtgggctccggcttcaagtgcaactccgccgtgtgggtggccctgcgcaacgtgaaggcctccgccaactccccctgggagcactgcatccacaagtaccccgtgcagatgtactccggctcctccaagtccgagacccgcgcccagaacggccgctcctgacttaag

SEQ ID NO: 37 - DNA, 1533 bp, Artificial Sequence
  Description of Artificial Sequence Synthetic polynucleotide
actagtatgacctccatcaacgtgaagctgctgtaccactacgtgctgaccaacttcttcaacctgtgcctgttccccctgaccgccttccccgccggcaaggcctcccagctgaccaccaacgacctgcaccacctgtactcctacctgcaccacaacctgatcaccgtgaccctgctgttcgccttcaccgtgttcggctccatcctgtacatcgtgacccgccccaagcccgtgtacctggtggactactcctgctacctgcccccccgccacctgtcctgcggcatctcccgcgtgatggagatcttctacgagatccgcaagtccgacccctcccgcgaggtgcccttcgacgacccctcctccctggagttcctgcgcaagatccaggagcgctccggcctgggcgacgagacctacggcccccagggcctggtgcacgacatgcccctgcgcatgaacttcgccgccgcccgcgaggagaccgagcaggtgatcaacggcgccctggagaagctgttcgagaacaccaaggtgaacccccgcgagatcggcatcctggtggtgaactcctccatgttcaaccccaccccctccctgtccgccatggtggtgaacaccttcaagctgcgctccaacatcaagtccttctccctgggcggcatgggctgctccgccggcatcatcgccatcgacctggccaaggacctgctgcacgtgcacaagaacacctacgccctggtggtgtccaccgagaacatcacccactccacctacaccggcgacaaccgctccatgatggtgtccaactgcctgttccgcatgggcggcgccgccatcctgctgtccaacaaggccggcgaccgccgccgctccaagtacaagctggcccacaccgtgcgcacccacaccggcgccgacgaccagtccttccgctgcgtgcgccaggaggacgacgaccgcggcaagatcggcgtgtgcctgtccaaggacatcaccgccgtggccggcaagaccgtgaccaagaacatcgccaccctgggccccctggtgctgcccctgtccgagaagttcctgtacgtggtgtccctgatggccaagaagctgttcaagaacaagatcaagcacacctacgtgcccgacttcaagctggccatcgaccacttctgcatccacgccggcggccgcgccgtgatcgacgtgctggagaagaacctggccctgtcccccgtggacgtggaggcctcccgctccaccctgcaccgcttcggcaacacctcctcctcctccatctggtacgagctggcctacatcgaggccaagggccgcatgaagaagggcaacaaggtgtggcagatcgccatcggctccggcttcaagtgcaactccgccgtgtgggtggccctgtgcaacgtgaagccctccgtgaactccccctgggagcactgcatcgaccgctaccccgtggagatcaactacggctcctccaagtccgagacccgcgcccagaacggccgctcctgacttaag

SEQ ID NO: 38 - DNA, 1524 bp, Artificial Sequence
  Description of Artificial Sequence Synthetic polynucleotide
actagtatgtccggcaccaaggccacctccgtgtccgtgcccctgcccgacttcaagcagtccgtgaacctgaagtacgtgaagctgggctaccactactccatcacccacgccatgtacctgttcctgacccccctgctgctgatcatgtccgcccagatctccaccttctccatccaggacttccaccacctgtacaaccacctgatcctgcacaacctgtcctccctgatcctgtgcatcgccctgctgctgttcgtgctgaccctgtacttcctgacccgccccacccccgtgtacctgctgaacttctcctgctacaagcccgacgccatccacaagtgcgaccgccgccgcttcatggacaccatccgcggcatgggcacctacaccgaggagaacatcgagttccagcgcaaggtgctggagcgctccggcatcggcgagtcctcctacctgccccccaccgtgttcaagatccccccccgcgtgtacgacgccgaggagcgcgccgaggccgagatgctgatgttcggcgccgtggacggcctgttcgagaagatctccgtgaagcccaaccagatcggcgtgctggtggtgaactgcggcctgttcaaccccatcccctccctgtcctccatgatcgtgaaccgctacaagatgcgcggcaacgtgttctcctacaacctgggcggcatgggctgctccgccggcgtgatctccatcgacctggccaaggacctgctgcaggtgcgccccaactcctacgccctggtggtgtccctggagtgcatctccaagaacctgtacctgggcgagcagcgctccatgctggtgtccaactgcctgttccgcatgggcggcgccgccatcctgctgtccaacaagatgtccgaccgctggcgctccaagtaccgcctggtgcacaccgtgcgcacccacaagggcaccgaggacaactgcttctcctgcgtgacccgcaaggaggactccgacggcaagatcggcatctccctgtccaagaacctgatggccgtggccggcgacgccctgaagaccaacatcaccaccctgggccccctggtgctgcccatgtccgagcagctgctgttcttcgccaccctggtgggcaagaaggtgttcaagatgaagctgcagccctacatccccgacttcaagctggccttcgagcacttctgcatccacgccggcggccgcgccgtgctggacgagctggagaagaacctgaagctgtcctcctggcacatggagccctcccgcatgtccctgtaccgcttcggcaacacctcctcctcctccctgtggtacgagctggcctactccgaggccaagggccgcatcaagaagggcgaccgcgtgtggcagatcgccttcggctccggcttcaagtgcaactccgccgtgtggaaggccctgcgcaacgtgaaccccgccgaggagaagaacccctggatggacgagatccacctgttccccgtggaggtgcccctgaactgacttaag

SEQ ID NO: 39 - DNA, 1530 bp, Artificial Sequence
  Description of Artificial Sequence Synthetic polynucleotide
actagtatgacctccatcaacgtgaagctgctgtaccactacgtgatcaccaacctgttcaacctgtgcttcttccccctgaccgccatcgtggccggcaaggcctacctgaccatcgacgacctgcaccacctgtactactcctacctgcagcacaacctgatcaccatcgcccccctgctggccttcaccgtgttcggctccgtgctgtacatcgccacccgccccaagcccgtgtacctggtggagtactcctgctacctgccccccacccactgccgctcctccatctccaaggtgatggacatcttcttccaggtgcgcaaggccgacccctcccgcaacggcacctgcgacgactcctcctggctggacttcctgcgcaagatccaggagcgctccggcctgggcgacgagacccacggccccgagggcctgctgcaggtgcccccccgcaagaccttcgcccgcgcccgcgaggagaccgagcaggtgatcatcggcgccctggagaacctgttcaagaacaccaacgtgaaccccaaggacatcggcatcctggtggtgaactcctccatgttcaaccccaccccctccctgtccgccatggtggtgaacaccttcaagctgcgctccaacgtgcgctccttcaacctgggcggcatgggctgctccgccggcgtgatcgccatcgacctggccaaggacctgctgcacgtgcacaagaacacctacgccctggtggtgtccaccgagaacatcacctacaacatctacgccggcgacaaccgctccatgatggtgtccaactgcctgttccgcgtgggcggcgccgccatcctgctgtccaacaagccccgcgaccgccgccgctccaagtacgagctggtgcacaccgtgcgcacccacaccggcgccgacgacaagtccttccgctgcgtgcagcagggcgacgacgagaacggccagaccggcgtgtccctgtccaaggacatcaccgacgtggccggccgcaccgtgaagaagaacatcgccaccctgggccccctgatcctgcccctgtccgagaagctgctgttcttcgtgaccttcatgggcaagaagctgttcaaggacgagatcaagcactactacgtgcccgacttcaagctggccatcgaccacttctgcatccacgccggcggcaaggccgtgatcgacgtgctggagaagaacctgggcctggcccccatcgacgtggaggcctcccgctccaccctgcaccgcttcggcaacacctcctcctcctccatctggtacgagctggcctacatcgagcccaagggccgcatgaagaagggcaacaaggtgtggcagatcgccctgggctccggcttcaagtgcaactccgccgtgtgggtggccctgaacaacgtgaaggcctccaccaactccccctgggagcactgcatcgaccgctaccccgtgaagatcgactccgactccggcaagtccgagacccgcgtgcccaacggccgctcctgacttaag

SEQ ID NO: 40 - DNA, 1599 bp, Artificial Sequence
  Description of Artificial Sequence Synthetic polynucleotide
actagtatggagcgcaccaactccatcgagatggaccaggagcgcctgaccgccgagatggccttcaaggactcctcctccgccgtgatccgcatccgccgccgcctgcccgacttcctgacctccgtgaagctgaagtacgtgaagctgggcctgcacaactccttcaacttcaccaccttcctgttcctgctgatcatcctgcccctgaccggcaccgtgctggtgcagctgaccggcctgaccttcgagaccttctccgagctgtggtacaaccacgccgcccagctggacggcgtgacccgcctggcctgcctggtgtccctgtgcttcgtgctgatcatctacgtgaccaaccgctccaagcccgtgtacctggtggacttctcctgctacaagcccgaggacgagcgcaagatgtccgtggactccttcctgaagatgaccgagcagaacggcgccttcaccgacgacaccgtgcagttccagcagcgcatctccaaccgcgccggcctgggcgacgagacctacctgccccgcggcatcacctccaccccccccaagctgaacatgtccgaggcccgcgccgaggccgaggccgtgatgttcggcgccctggactccctgttcgagaagaccggcatcaagcccgccgaggtgggcatcctgatcgtgtcctgctccctgttcaaccccaccccctccctgtccgccatgatcgtgaaccactacaagatgcgcgaggacatcaagtcctacaacctgggcggcatgggctgctccgccggcctgatctccatcgacctggccaacaacctgctgaaggccaaccccaactcctacgccgtggtggtgtccaccgagaacatcaccctgaactggtacttcggcaacgaccgctccatgctgctgtgcaactgcatcttccgcatgggcggcgccgccatcctgctgtccaaccgccgccaggaccgctccaagtccaagtacgagctggtgaacgtggtgcgcacccacaagggctccgacgacaagaactacaactgcgtgtaccagaaggaggacgagcgcggcaccatcggcgtgtccctggcccgcgagctgatgtccgtggccggcgacgccctgaagaccaacatcaccaccctgggccccatggtgctgcccctgtccggccagctgatgttctccgtgtccctggtgaagcgcaagctgctgaagctgaaggtgaagccctacatccccgacttcaagctggccttcgagcacttctgcatccacgccggcggccgcgccgtgctggacgaggtgcagaagaacctggacctggaggactggcacatggagccctcccgcatgaccctgcaccgcttcggcaacacctcctcctcctccctgtggtacgagatggcctacaccgaggccaagggccgcgtgaaggccggcgaccgcctgtggcagatcgccttcggctccggcttcaagtgcaactccgccgtgtggaaggccctgcgcgtggtgtccaccgaggagctgaccggcaacgcctgggccggctccatcgagaactaccccgtgaagatcgtgcagtgacttaag

SEQ ID NO: 41 - DNA, 5851 bp, Artificial Sequence
  Description of Artificial Sequence Synthetic polynucleotide
gctcttcggagtcactgtgccactgagttcgactggtagctgaatggagtcgctgctccactaaacgaattgtcagcaccgccagccggccgaggacccgagtcatagcgagggtagtagcgcgccatggcaccgaccagcctgcttgccagtactggcgtctcttccgcttctctgtggtcctctgcgcgctccagcgcgtgcgcttttccggtggatcatgcggtccgtggcgcaccgcagcggccgctgcccatgcagcgccgctgcttccgaacagtggcggtcagggccgcacccgcggtagccgtccgtccggaacccgcccaagagttttgggagcagcttgagccctgcaagatggcggaggacaagcgcatcttcctggaggagcaccggtgcgtggaggtccggggctgaccggccgtcgcattcaacgtaatcaatcgcatgatgatcagaggacacgaagtcttggtggcggtggccagaaacactgtccattgcaagggcatagggatgcgttccttcacctctcatttctcatttctgaatccctccctgctcactctttctcctcctccttcccgttcacgcagcattcggggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacgtagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagatatcgcgaggggtctgcctgggccagccgctccctctaaacacgggacgcgtggtccaattcgggcttcgggaccctttggcggtttgaacgccagggatggggcgcccgcgagcctggggaccccggcaacggcttccccagagcctgccttgcaatctcgcgcgtcctctccctcagcacgtggcggttccacgtgtggtcgggcttcccggactagctcgcgtcgtgacctagcttaatgaacccagccgggcctgtagcaccgcctaagaggttttgattatttcattataccaatctattcgccactagtatggccatcaagaccaaccgccagcccgtggagaagccccccttcaccatcggcaccctgcgcaaggccatccccgcccactgcttcgagcgctccgccctgcgctcctccatgtacctggccttcgacatcgccgtgatgtccctgctgtacgtggcctccacctacatcgaccccgcccccgtgcccacctgggtgaagtacggcgtgatgtggcccctgtactggttcttccagggcgccttcggcaccggcgtgtgggtgtgcgcccacgagtgcggccaccaggccttctcctcctcccaggccatcaacgacggcgtgggcctggtgttccactccctgctgctggtgccctactactcctggaagcactcccaccgccgccaccactccaacaccggctgcctggacaaggacgaggtgttcgtgcccccccaccgcgccgtggcccacgagggcctggagtgggaggagtggctgcccatccgcatgggcaaggtgctggtgaccctgaccctgggctggcccctgtacctgatgttcaacgtggcctcccgcccctacccccgcttcgccaaccacttcgacccctggtcccccatcttctccaagcgcgagcgcatcgaggtggtgatctccgacctggccctggtggccgtgctgtccggcctgtccgtgctgggccgcaccatgggctgggcctggctggtgaagacctacgtggtgccctacctgatcgtgaacatgtggctggtgctgatcaccctgctgcagcacacccaccccgccctgccccactacttcgagaaggactgggactggctgcgcggcgccatggccaccgtggaccgctccatgggcccccccttcatggacaacatcctgcaccacatctccgacacccacgtgctgcaccacctgttctccaccatcccccactaccacgccgaggaggcctccgccgccatccgccccatcctgggcaagtactaccagtccgactcccgctgggtgggccgcgccctgtgggaggactggcgcgactgccgctacgtggtgcccgacgcccccgaggacgactccgccctgtggttccacaagtagatcgatcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcttgttttccagaaggagttgctccttgagcctttcattctcagcctcgataacctccaaagccgctctaattgtggagggggttcgaatttaaaagcttggaatgttggttcgtgcgtctggaacaagcccagacttgttgctcactgggaaaaggaccatcagctccaaaaaacttgccgctcaaaccgcgtacctctgctttcgcgcaatctgccctgttgaaatcgccaccacattcatattgtgacgcttgagcagtctgtaattgcctcagaatgtggaatcatctgccccctgtgcgagcccatgccaggcatgtcgcgggcgaggacacccgccactcgtacagcagaccattatgctacctcacaatagttcataacagtgaccatatttctcgaagctccccaacgagcacctccatgctctgagtggccaccccccggccctggtgcttgcggagggcaggtcaaccggcatggggctaccgaaatccccgaccggatcccaccacccccgcgatgggaagaatctctccccgggatgtgggcccaccaccagcacaacctgctggcccaggcgagcgtcaaaccataccacacaaatatccttggcatcggccctgaattccttctgccgctctgctacccggtgcttctgtccgaagcaggggttgctagggatcgctccgagtccgcaaacccttgtcgcgtggcggggcttgttcgagcttgaagagc

SEQ ID NO: 42 - DNA, 186 bp, Artificial Sequence
  Description of Artificial Sequence Synthetic polynucleotide
tacaacttattacgtaacggagcgtcgtgcgggagggagtgtgccgagcggggagtcccggtctgtgcgaggcccggcagctgacgctggcgagccgtacgccccgagggtccccctcccctgcaccctcttccccttccctctgacggccgcgcctgttcttgcatgttcagcgacgaggatatc

SEQ ID NO: 43 - DNA, 305 bp, Artificial Sequence
  Description of Artificial Sequence Synthetic polynucleotide
gcgaggggtctgcctgggccagccgctccctctgaacacgggacgcgtggtccaattcgggcttcgggaccctttggcggtttgaacgcctgggagagggcgcccgcgagcctggggaccccggcaacggcttccccagagcctgccttgcaatctcgcgcgtcctctccctcagcacgtggcggttccacgtgtggtcgggcgtcccggactagctcacgtcgtgacctagcttaatgaacccagccgggcctgcagcaccaccttagaggttttgattatttgattagaccaatctattcacc

SEQ ID NO: 44 - DNA, 305 bp, Artificial Sequence
  Description of Artificial Sequence Synthetic polynucleotide
ggcgaatagattggtataatgaaataatcaaaacctcttaggcggtgctacaggcccggctgggttcattaagctaggtcacgacgcgagctagtccgggaagcccgaccacacgtggaaccgccacgtgctgagggagaggacgcgcgagattgcaaggcaggctctggggaagccgttgccggggtccccaggctcgcgggcgccccatccctggcgttcaaaccgccaaagggtcccgaagcccgaattggaccacgcgtcccgtgtttagagggagcggctggcccaggcagacccctcgc

SEQ ID NO: 45 - DNA, 305 bp, Artificial Sequence
  Description of Artificial Sequence Synthetic polynucleotide
ggtgaatagattggtctaatcaaataatcaaaacctctaaggtggtgctgcaggcccggctgggttcattaagctaggtcacgacgtgagctagtccgggacgcccgaccacacgtggaaccgccacgtgctgagggagaggacgcgcgagattgcaaggcaggctctggggaagccgttgccggggtccccaggctcgcgggcgccctctcccaggcgttcaaaccgccaaagggtcccgaagcccgaattggaccacgcgtcccgtgttcagagggagcggctggcccaggcagacccctcgc

SEQ ID NO: 46 - DNA, 1322 bp, Artificial Sequence
  Description of Artificial Sequence Synthetic polynucleotide
gtgatgggttctttagacgatccagcccaggatcatgtgttgcccacatggagcctatccacgctggcctagaaggcaagcacatttcaaggtgaacccacgtccatggagcgatggcgccaatatctcgcctctagaccaagcggttctcaccccaactgcgtcatttgtatgtatggctgcaaagttgtcggtacgatagaggccgccaacctggcggcgagggcgaggagctggttgccgatctgtgcccaagcatgtgtcggagctcggctgtctcggcagcgagctcctgtgcaaggggcttgcatcgagaatgtcaggcgatagacactgcacgttggggacacggaggtgcccctgtggcgtgtcctggatgccctcgggtccgtcgcgagaagctctggcgaccagcacccggccacaaccgcagcaggcgttcacccacaagaatcttccagatcgtgatgcgcatgtatcgtgacacgattggcgaggtccgcaggacgcacacggactcgtccactcatcagaactggtcagggcacccatctgcgtcccttttcaggaaccacccaccgctgccaggcaccttcgccagcggcggactccacacagagaatgccttgctgtgagagaccatggccggcaagtgctgtcggatctgcccgcatacggtcagtccccagcacaaggaagccaagagtacaggctgttggtgtcgatggaggagtggccgttcccacaagtagtgagcggcagctgctcaacggcttccccctgttcatcttggcaaagccagtgacttcctacaagtatgtgatgcagatcggcactgcaatctgtcggcatgcgtacagaacatcggctcgccagggcagcgttgctcgctctggatgagctgcttgggaggaatcatcggcacacgcccgtgccgtgcccgcgccccgcgcccgtcgggaaaggcccccggttaggacactgccgcgtcagccagtcgtgggatcgatcggacgtggcgaatcctcgcccggacaccctcatcacaccccacatttccctgcaagcaatcttgccgacaaaatagtcaagatccattgggtttagggaacacgtgcgagactgggcagctgtatctgtccttgccccgcgtcaaattcctgggcgtgacgcagtcacaggagaatctattagaccctggacttgcagctcagtcatgggcgtgagtggctaaagcacctaggtcaggcgagtaccgccccttccccaggattcactcttctgcgattgacgttgagcctgcatcgggctgcttcgtcacc

SEQ ID NO: 47 - DNA, 841 bp, Artificial Sequence
  Description of Artificial Sequence Synthetic polynucleotide
tcggagctaaagcagagactggacaagacttgcgttcgcatactggtgacacagaatagctcccatctattcatacgcctttgggaaaaggaacgagccttgtggcctctgcattgctgcctgctttgaggccgaggacggtgcgggacgctcagatccatcagcgatcgccccaccctcagagcacctccgatccaaggcaatactatcaggcaaagtttccaaattcaaacattccaaaatcacgccagggactggatcacacacgcagatcagcgccgttttgctctttgcctacgggcgactgtgccacttgtcgacccctggtgacgggagggaccacgcctgcggttggcatccacttcgacggacccagggacggtttctcatgccaaacctgagatttgagcacccagatgagcacattatgcgttttaggatgcctgagcagcgggcgtgcaggaatctggtctcgccagattcaccgaagatgcgcccatcggagcgaggcgagggctttgtgaccacgcaaggcagtgtgaggcaaacacatagggacacctgcgtctttcaatgcacagacatctatggtgcccatgtatataaaatgggctacttctgagtcaaaccaacgcaaactgcgctatggcaaggccggccaaggttggaatcccggtctgtctggatttgagtttgtgggggctatcacgtgacaatccctgggattgggcggcagcagcgcacggcctgggtggcaatggcgcactaatactgctgaaagcacggctctgcatccctttctcttgacctgcgattggtccttttcgcaagcgtgatcatc

SEQ ID NO: 48 - DNA, 841 bp, Artificial Sequence
  Description of Artificial Sequence Synthetic polynucleotide
tcggagctaaagcagaaactgaacaagacttgcgttcgcatacttgtgacactgaataggttcaatctattcatacgcctttgggaaactgaacgagccttgtggcctctgcattgctgcctgctttgaggccgaggacggcgcggaacgcacagatccatcagcgatcgccccaccctcagagtacatccgatccaaggcaatactatcaggcaaagtttccaaattcaaacattccaaaattacgtcagggactggatcacacacgcagatcagcgccgttttgctctttgcctacgggcgactgtgccacttgtcgacgcctggtgacgggagggaccacgcctgcggttggcatccacttcgacggacccagggacggtctcacatgccaaacctgagatttgagcaccaagatgagcacattatgcgtttttggatgcctgagcagcgggcgtgcaggaatctggtctcgccagattcaccgaagatgcggccatcggagcgaggcgagggctgtgtggccacgccaggcagtgtgaggcaaacacacagggacatctgcttctttcgatgcacagacatctatgttgcccgtgcatataaaatgggctacttctgaatcaaaccaacgcaaacttcgctatggcaaggccggccaaggttggaatcccggtctgtctggatttgagtttgtgggggctatcacgtgacaatccctgggattgggcggcagcagcgcacggcctggatggcaatggcgcactaatactgctgaaagcacggctctgcatccctttctcttgacctgcgattggtccttttcgcaagcgtgatcatc

SEQ ID NO: 49 - DNA, 512 bp, Artificial Sequence
  Description of Artificial Sequence Synthetic polynucleotide
caccgatcactccgtcgccgcccaagagaaatcaacctcgatggagggcgaggtggatcagaggtattggttatcgttcgttcttagtctcaatcaatcgtacaccttgcagttgcccgagtttctccacacatacagcacctcccgctcccagcccattcgagcgacccaatccgggcgatcccagcgatcgtcgtcgcttcagtgctgaccggtggaaagcaggagatctcgggcgagcaggaccacatccagcccaggatcttcgactggctcagagctgaccctcacgcggcacagcaaaagtagcacgcacgcgttatgcaaactggttacaacctgtccaacagtgttgcgacgttgactggctacattgtctgtctgtcgcgagtgcgcctgggcccttacggtgggacactggaactccgccccgagtcgaacacctagggcgacgcccgcagcttggcatgacagctctccttgtgttctaaataccttgcgcgtgtgggaga

SEQ ID NO: 50 - DNA, 516 bp, Artificial Sequence
  Description of Artificial Sequence Synthetic polynucleotide
atccaccgatcactccgtcgccgcccaagagaattcaacctcgatggagggcaaggtggatcagaggtattggttatcgttcgctattagtctcaatcaatcgtgcaccttgcagttgctcgagtttctccacacatacagcacctcccgctcccagcccattcgagcgacccaatccgggcgatcccagcgatcgtcgtcgcttcagtgctgaccggtggaaagcaggagatctcgggcgagcaggaccacatccagcacaggatcttcgactggctcagagctgaccctcacgcggcacagcaaaagtagcccgcacgcgttatgcaaacaggttacaacctgtccaacactgttgcgacgttgactggctacattgtctgtctgtcgcgagtacgcctggacccttacggtgggacactggaactccgccccgagtcgaacacctagggcgacgcccgcagcttggcatgacagctctccttgtattctaaatacctcgcgcgtgtgggagaa

SEQ ID NO: 51 - DNA, 335 bp, Artificial Sequence
  Description of Artificial Sequence Synthetic polynucleotide
atgatgcgcgtgtacgactatcaaggaagaaagaggacttaatttcttaccttctaaccaccatattctttttgctggatgcttgctcgtctcgatgacaattgtgaacctcttgtgtgaccctgaccctgctgcaaggctctccgaccgcacgcaaggcgcagccggcgcgtccggaggcgatcggatccaatccagtcgtcctcccgcagcccgggcacgtttgcccatgcaggcccttccacaccgctcaagagactcccgaacaccgcccactcggcactcgcttcggctgccgagtgcgcgtttgagtttgccctgccacagaagacacc

SEQ ID NO: 52 - DNA, 335 bp, Artificial Sequence
  Description of Artificial Sequence Synthetic polynucleotide
atgatgcgcgtgtacgactatcaaggaagaaagaggacttaatttcttaccttctaaccaccatattctttttgctggatgcttgctcgtctcgatgacaattgtgaacctcttgtgtgaccctgaccctgctgcaaggctctccgaccgcacgcaaggcgcagccggcgcgtccggaggcgatcggatccaatccagtcgtcctcccgcagcccgggcacgtttgcccatgcaggcccttccacaccgctcaagagactcccgaacaccgcccactcggcactcgcttcggctgccgagtgcgcgtttgagtttgccctgccacaggagacatc

SEQ ID NO: 53 - DNA, 1097 bp, Artificial Sequence
  Description of Artificial Sequence Synthetic polynucleotide
cccgggcgagctgtacgcctacggagcgaggcctggtgtgaccgttgcgatctcgccagcagacgtcgcggagcctcgtcccaaaggccctttctgatcgagcttgtcgtccactggacgctttaagttgcgcgcgcgatgggataaccgagctgatctgcactcagattttggtttgttttcgcgcatggtgcagcgaggggaggtactacgctggggtacgagatcctccggattcccagaccgtgttgccggcatttacccggtcatcgccagcgattcgggacgacaaggccttatcctgtgctgagacgctcgagcacgtttataaaattgtgggtaccgcggtatgcacagcgttcaacacgcgccacgccgaaattggttggtgggggagcacgtatgggactgacgtatggccagcagcgaacactcaccgaacaagtgccaatgtataccttgcatcaatgatgctccggcagcttcgattgactgtctcgaaaaagtgtgagcaagcagatcatgtggccgctctgtcgcgcagcacctgacgcattcgacacccacggcaatgcccaggccagggaatagagagtaagacaactcccattgttcagcaaaacattgcactgcagtgccttcacaactatacaatgaatgggagggaatatgggctctgcatgggacagcttagctgggacattcggctactgaacaagaaaaccccacgagaaccaattggcgaaacctgccgggaggaggtgatcgtttctgtaaatggcttacgcattcccccccggcggctcacgaggggtgtggtgaaccctgccagctgatcaagtgcttgctgacgtcggccagggaggtgtatgtgattgggccgtggggcgtgagttatcctaccgccggacccgcgaagtcacatgacgaatggccgtgcgggatgacgagagcacgactcgctctttcttcgccggcccggcttcatggaggacaataataaagggtggccaccggcaacagccctccatacctgaaccgattccagacccaaacctcttgaattttgagggatccagttcaccggtatagtcacg

SEQ ID NO: 54 - DNA, 1105 bp, Artificial Sequence
  Description of Artificial Sequence Synthetic polynucleotide
atccccgggcgagctgtacgcctacggagcgaggcctggtgtgaccgttgcgatctcgccagcagacgtcgcggagcctcgtcccaaaggccctttctgatcgagcttgtcgtccactggacgctttaagttgcgcgcgcgatgggataaccgagctgatctgcactcagattttggtttgttttcgcgcatggtgcagcgaggggaggtactacgctggggtacgagatcctccggattcccagaccgtgttgccggcatttacccggtcatcgccagcgattcgggacgacaaggccttatcctgtgctgagacgctcgagcacgtttataaaattgtggtcaccgtggtacgcacagcgtccaacacgcgccacgccgaaattcgttggtgggggagcacgtatcggactgacgtatggccagcagcgaacactcaccaaacaggtgccaatgtatagcttgcatcaatgatgctctggcagcttcgattgactgtctcgaaaaagtgtgtgcaaacagattatgtggccgctctgtggccgcgcagcacctgacgcactcgacacccacggcaatgcccaggccaaggaacagagagtaagacaactcccattgttcagtaaaacattgcactgcagtgccttcacaaacatacaacgaatgggagggaatatgggcttcgaatgggacagcttagctgggacattcggttactgaacaagaaaaccccacgagaaccaactggcgaaacctgccgggaggaggtgatcgtttttgtaaatggcttacgcattccccccccggcggctcacggggggtgtggtgaaccctgccagctgatcaagtgcttgctgacgtcggccagggaggtgtatgtgatttggccgtggggcgtgagttatcctaccgccggacccgcgaagtcacatgacgaatggccgtgcgggatgacgagagcagggctcgctctttcttcgccggcccggcttcatggaggacaataataaagggtggccaccggcaacagccctccatacctgaaccgattccagacccaaacctcttgaattttgagggatccagttcaccggtatagtcacga

SEQ ID NO: 55 - DNA, 754 bp, Artificial Sequence
  Description of Artificial Sequence Synthetic polynucleotide
gcgagtggttttgctgccgggaagggagtggggagcgtcgagcgagggacgcggcgctcgaggcgcacgtcgtctgtcaacgcgcgcggccctcgcggcccgcggccccacccagctctaatcatcgaaaactaagaggctccacacgcctgtcgtagaatgcatgggattcgccagtagaccacgatctgcgccgaagaagctggtctacccgacgttttttgttgctcctttattctgaatgatatgaagatagtgtgcgcagtgccacgcataggcatcaggagcaagggaggacgggtcaacttgaaagaaccaaaccatccatccgagaaatgcgcatcatctttgtagtaccatcaaacgccttggccaatgtcttctgcatggacaacacaacctgctcctggccacacggtcgacttggagcgccccatgcgcccaggtcgccacgacccgcggcccagcgcgcggcgattcgcctcacgagatcccggcggacccggcacgcccgcgggccgacggtgcgcttggcgatgctgctcattaacccacggccgtcacccgatccacatgctctttttcaacacatccacattggaatagagctctaccagggtgagtactgcattctttggggctgggaggaccccactcgacacctggtccttcatcggccgaaagcccgaacctgagcgcttccccgccccgttcctcatccccgactttccgatggcccattgcagtttcaaac

SEQ ID NO: 56 - DNA, 318 bp, Artificial Sequence
  Description of Artificial Sequence Synthetic polynucleotide
atctgggtggaggactgggagtaagatgtaaggatattaattaaacattctagtttgttgatggcacaacagtcaatgcatttcagtcgtcttgctccttataacctatgcgtgtgccatcgccggccatgcacctgtggcgtggtaccgaccatcggggagaggcccgagattcggaggtacctcccgccctgggcgagcccttcacgtgacggcacaagtcccttgcatcggcccgcgagcacggaatacagagccccgtgccccccacgggccctcacatcatccactccattgttcttgccacaccgatcagca

SEQ ID NO: 57 - DNA, 316 bp, Artificial Sequence
  Description of Artificial Sequence Synthetic polynucleotide
tgggtggaggactgggaagaagatgtaaggatatcaatttaacattctagtttgttgatggcacaacagtcactgaataccgggcgtctggctgctaaaatagccggagcgtgtgccatcgccggccatgcatctgtggcgtggtaccgaccatcagggagaggcccgagattcggaggtacctcccgccctgggcgagcccttcacgtgacggcacaagtcccttgcatcggcccgcgagcacggaatacagagccccgtgctccccacgggccctcacatcatccactccattgttcttgccacaccgatcagc

SEQ ID NO: 58 - DNA, 350 bp, Artificial Sequence
  Description of Artificial Sequence Synthetic polynucleotide
ataacgaggcacaatgatcgatatttctatcgaacaactgtatttagccctgtacgtaccccgctcttgggccagcccgtccgtgcttgccttcggaaaattgcatggcgcctcatgcaaactcgcgctctcacagcagatctcgcccagctcccgggagagcaatcgcgggtggggcccggggcgaatccaggacgcgccccgcggggccgctccactcgccagggccaatgggcggcttatagtcctggcatgggctctgcatgcacagtatcgcagtttgggcgaggtgttgcccccgcgatttcgaatacgcgacgcccggtactcgtgcgagaacagggttcttg

SEQ ID NO: 59 - DNA, 818 bp, Artificial Sequence
  Description of Artificial Sequence Synthetic polynucleotide
atcgcgatggtgcgcactcgtgcgcaatgaatatggggtcacgcggtggacgaacgcggagggggcctggccgaatctaggcttgcattcctcagatcactttctgccggcggtccggggtttgcgcgtcgcgcaacgctccgtctccctagccgctgcgcaccgcgcgtgcgacgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggcccactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacgcccagca

SEQ ID NO: 60 - DNA, 819 bp, Artificial Sequence
  Description of Artificial Sequence Synthetic polynucleotide
atcacgatggtgcgcattcgtgcaaagtgaatatggggtcacgcggtggacgaacgcggagggggcatgaccgaatctaggctcgcattcctcagatcacttcatgccggcggtccggggtttgcgcgtcgcgcaaggctacgtctccctagccgctgcgcaccacgcgtgcgacgcggaggccatcttccggagcaacgaccatggattgtcttagcgatcgcacgaatgagtgctagtgagtcgtacgctcgacccagtcgctcgcaggagaaggcggcagctgccgagcttcggcttaccagtcgtgactcgtatgtgatcaggaatcattggcattggtagcattataattcggcttccgcgctgcgtatgggcatggcaatgtctcatgcagtcgatcttagtcaaccaattttgggtggccaggtccgggcgaccgggctccgtgtcgccgggcaccacctcctgccaggagtagcagggccgccctctcgtcccgacgttggcccactgaataccgtggcttcgagccctacatgatgggctgcctagtcgggcgggacgcgcaactgcccgcgcgatctgggggctggtctgaatccttcaggcgggtgttacccgagaaagaaagggtgccgatttcaaagcagacccatgtgccgggccctgtggcctgtgttggcgcctatgtagtcaccccccctcacccaattgtcgccagtttgcgcactccataaactcaaaacagcagcttctgagctgcgctgttcaagaacacctctggggtttgctcacccgcgaggtcgacgcccagca

SEQ ID NO: 61 - DNA, 7081 bp, Artificial Sequence
  Description of Artificial Sequence Synthetic polynucleotide
gctcttcgccgccgccactcctgctcgagcgcgcccgcgcgtgcgccgccagcgccttggccttttcgccgcgctcgtgcgcgtcgctgatgtccatcaccaggtccatgaggtctgccttgcgccggctgagccactgcttcgtccgggcggccaagaggagcatgagggaggactcctggtccagggtcctgacgtggtcgcggctctgggagcgggccagcatcatctggctctgccgcaccgaggccgcctccaactggtcctccagcagccgcagtcgccgccgaccctggcagaggaagacaggtgaggggggtatgaattgtacagaacaaccacgagccttgtctaggcagaatccctaccagtcatggctttacctggatgacggcctgcgaacagctgtccagcgaccctcgctgccgccgcttctcccgcacgcttctttccagcaccgtgatggcgcgagccagcgccgcacgctggcgctgcgcttcgccgatctgaggacagtcggggaactctgatcagtctaaacccccttgcgcgttagtgttgccatcctttgcagaccggtgagagccgacttgttgtgcgccaccccccacaccacctcctcccagaccaattctgtcacctttttggcgaaggcatcggcctcggcctgcagagaggacagcagtgcccagccgctgggggttggcggatgcacgctcaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcttgttttccagaaggagttgctccttgagcctttcattctcagcctcgataacctccaaagccgctctaattgtggagggggttcgaatttaaaagcttggaatgttggttcgtgcgtctggaacaagcccagacttgttgctcactgggaaaaggaccatcagctccaaaaaacttgccgctcaaaccgcgtacctctgctttcgcgcaatctgccctgttgaaatcgccaccacattcatattgtgacgcttgagcagtctgtaattgcctcagaatgtggaatcatctgccccctgtgcgagcccatgccaggcatgtcgcgggcgaggacacccgccactcgtacagcagaccattatgctacctcacaatagttcataacagtgaccatatttctcgaagctccccaacgagcacctccatgctctgagtggccaccccccggccctggtgcttgcggagggcaggtcaaccggcatggggctaccgaaatccccgaccggatcccaccacccccgcgatgggaagaatctctccccgggatgtgggcccaccaccagcacaacctgctggcccaggcgagcgtcaaaccataccacacaaatatccttggcatcggccctgaattccttctgccgctctgctacccggtgcttctgtccgaagcaggggttgctagggatcgctccgagtccgcaaacccttgtcgcgtggcggggcttgttcgagcttgaagagc

SEQ ID NO: 62 - DNA, 8286 bp, Artificial Sequence
  Description of Artificial Sequence Synthetic polynucleotide
gctcttcccaactcagataataccaatacccctccttctcctcctcatccattcagtacccccccccttctcttcccaaagcagcaagcgcgtggcttacagaagaacaatcggcttccgccaaagtcgccgagcactgcccgacggcggcgcgcccagcagcccgcttggccacacaggcaacgaatacattcaatagggggcctcgcagaatggaaggagcggtaaagggtacaggagcactgcgcacaaggggcctgtgcaggagtgactgactgggcgggcagacggcgcaccgcgggcgcaggcaagcagggaagattgaagcggcagggaggaggatgctgattgaggggggcatcgcagtctctcttggacccgggataaggaagcaaatattcggccggttgggttgtgtgtgtgcacgttttcttcttcagagtcgtgggtgtgcttccagggaggatataagcagcaggatcgaatcccgcgaccagcgtttccccatccagccaaccaccctgtcggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagcatagcgactgctaccccccgaccatgtgccgaggcagaaattatatacaagaagcagatcgcaattaggcacatcgctttgcattatccacacactattcatcgctgctgcggcaaggctgcagagtgtatttttgtggcccaggagctgagtccgaagtcgacgcgacgagcggcgcaggatccgacccctagacgagctctgtcattttccaagcacgcagctaaatgcgctgagaccgggtctaaatcatccgaaaagtgtcaaaatggccgattgggttcgcctaggacaatgcgctgcggattcgctcgagtccgctgccggccaaaaggcggtggtacaggaaggcgcacggggccaaccctgcgaagccgggggcccgaacgccgaccgccggccttcgatctcgggtgtccccctcgtcaatttcctctctcgggtgcagccacgaaagtcgtgacgcaggtcacgaaatccggttacgaaaaacgcaggtcttcgcaaaaacgtgagggtttcgcgtctcgccctagctattcgtatcgccgggtcagacccacgtgcagaaaagcccttgaataacccgggaccgtggttaccgcgccgcctgcaccagggggcttatataagcccacaccacacctgtctcaccacgcatttctccaactcgcgacttttcggaagaaattgttatccacctagtatagactgccacctgcaggaccttgtgtcttgcagtttgtattggtcccggccgtcgagctcgacagatctgggctagggttggcctggccgctcggcactcccctttagccgcgcgcatccgcgttccagaggtgcgattcggtgtgtggagcattgtcatgcgcttgtgggggtcgttccgtgcgcggcgggtccgccatgggcgccgacctgggccctagggtttgttttcgggccaagcgagcccctctcacctcgtcgcccccccgcattccctctctcttgcagcccatatggccatggccgccgccgtgatcgtgcccctgggcatcctgttcttcatctccggcctggtggtgaacctgctgcaggccatctgctacgtgctgatccgccccctgtccaagaacacctaccgcaagatcaaccgcgtggtggccgagaccctgtggctggagctggtgtggatcgtggactggtgggccggcgtgaagatccaggtgttcgccgacaacgagaccttcaaccgcatgggcaaggagcacgccctggtggtgtgcaaccaccgctccgacatcgactggctggtgggctggatcctggcccagcgctccggctgcctgggctccgccctggccgtgatgaagaagtcctccaagttcctgcccgtgatcggctggtccatgtggttctccgagtacctgttcctggagcgcaactgggccaaggacgagtccaccctgaagtccggcctgcagcgcctgaacgacttcccccgccccttctggctggccctgttcgtggagggcacccgcttcaccgaggccaagctgaaggccgcccaggagtacgccgcctcctccgagctgcccgtgccccgcaacgtgctgatcccccgcaccaagggcttcgtgtccgccgtgtccaacatgcgctccttcgtgcccgccatctacgacatgaccgtggccatccccaagacctcccccccccccaccatgctgcgcctgttcaagggccagccctccgtggtgcacgtgcacatcaagtgccactccatgaaggacctgcccgagtccgacgacgccatcgcccagtggtgccgcgaccagttcgtggccaaggacgccctgctggacaagcacatcgccgccgacaccttccccggccagcaggagcagaacatcggccgccccatcaagtccctggccgtggtgctgtcctggtcctgcctgctgatcctgggcgccatgaagttcctgcactggtccaacctgttctcctcctggaagggcatcgccttctccgccctgggcctgggcatcatcaccctgtgcatgcagatcctgatccgctcctcccagtccgagcgctccacccccgccaaggtggtgcccgccaagcccaaggacaaccacaacgactccggctcctcctcccagaccgaggtggagaagcagaagtgaatgcatgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactagatatcatgtggatgatgagcatgaattcctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacactagtatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcaccggtgcctacgtgggtcaagtacggcatcatgtggccgctctactggttcttccaggtgtgtttgagggttttggttgcccgtattgaggtcctggtggcgcgcatggaggagaaggcgcctgtcccgctgacccccccggctaccctcccggcaccttccagggcgcgtacgggaagaaccagtagagcggccacatgatgccgtacttgacccacgtaggcaccggtgcagggtcgatgtacgtcgacgcgacgtagagcagggacatgaccgcgatgtcaaaggccaggtacatgctgctacgaagcgccgagcgctcgaaacagtgcgcggggatggccttgcgcagcgtcccgatcgtgaacggaggcttctccacaggctgcctgttcgtcttgatagccatctcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagagctcttgttttccagaaggagttgctccttgagcctttcattctcagcctcgataacctccaaagccgctctaattgtggagggggttcgaaccgaatgctgcgtgaacgggaaggaggaggagaaagagtgagcagggagggattcagaaatgagaaatgagaggtgaaggaacgcatccctatgcccttgcaatggacagtgtttctggccaccgccaccaagacttcgtgtcctctgatcatcatgcgattgattacgttgaatgcgacggccggtcagccccggacctccacgcaccggtgctcctccaggaagatgcgcttgtcctccgccatcttgcagggctcaagctgctcccaaaactcttgggcgggttccggacggacggctaccgcgggtgcggccctgaccgccactgttcggaagcagcggcgctgcatgggcagcggccgctgcggtgcgccacggaccgcatgatccaccggaaaagcgcacgcgctggagcgcgcagaggaccacagagaagcggaagagacgccagtactggcaagcaggctggtcggtgccatggcgcgctactaccctcgctatgactcgggtcctcggccggctggcggtgctgacaattcgtttagtggagcagcgactccattcagctaccagtcgaactcagtggcacagtgactccgctcttc

SEQ ID NO: 63 - protein, 390 aa, Brassica napus
MAMAAAVIVPLGILFFISGLVVNLLQAVCYVLVRPMSKNTYRKINRVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLQSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPEPEDEIAQWCRDQFVAKDALLDKHIAADTFPGQKEQNIGRPIKSLAVVVSWACLLTLGAMKFLHWSNLFSSWKGIALSAFGLGIITLCMQILIRSSQSERSTPAKVAPAKPKDNHQSGPSSQTEVEEKQK

SEQ ID NO: 64 - protein, 431 aa, Protheca moriformis
AAAAADANPARPERRVVITGQGVVTSLGQTIEQFYSSLLEGVSGISQIQKFDTTGYTTTIAGEIKSLQLDPYVPKRWAKRVDDVIKYVYIAGKQALESAGLPIEAAGLAGAGLDPALCGVLIGTAMAGMTSFAAGVEALTRGGVRKMNPFCIPFSISNMGGAMLAMDIGFMGPNYSISTACATGNYCILGAADHIRRGDANVMLAGGADAAIIPSGIGGFIACKALSKRNDEPERASRPWDADRDGFVMGEGAGVLVLEELEHAKRRGATILAELVGGAATSDAHHMTEPDPQGRGVRLCLERALERARLAPERVGYVNAHGTSTPAGDVAEYRAIRAVIPQDSLRINSTKSMIGHLLGGAGAVEAVAAIQALRTGWLHPNLNLENPAPGVDPVVLVGPRKERAEDLDVVLSNSFGFGGHNSCVIFRKYDE

SEQ ID NO: 65 - protein, 387 aa, Prototheca moriformis
GAVAAPGRRAASRPLVVHAVASEAPLGVPPSVQRPSPVVYSKLDKQHRLTPERLELVQSMGQFAEERVLPVLHPVDKLWQPQDFLPDPESPDFEDQVAELRARAKDLPDEYFVVLVGDMITEEALPTYMAMLNTLDGVRDDTGAADHPWARWTRQWVAEENRHGDLLNKYCWLTGRVNMRAVEVTINNLIKSGMNPQTDNNPYLGFVYTSFQERATKYSHGNTARLAAEHGDKGLSKICGLIASDEGRHEIAYTRIVDEFFRLDPEGAVAAYANMMRKQITMPAHLMDDMGHGEANPGRNLFADFSAVAEKIDVYDAEDYCRILEHLNARWKVDERQVSGQAAADQEYVLGLPQRFRKLAEKTAAKRKRVARRPVAFSWISGREIMV

SEQ ID NO: 66 - DNA, 7137 bp, Artificial Sequence
  Description of Artificial Sequence Synthetic polynucleotide
gctcttcacccaactcagataataccaatacccctccttctcctcctcatccattcagtacccccccccttctcttcccaaagcagcaagcgcgtggcttacagaagaacaatcggcttccgccaaagtcgccgagcactgcccgacggcggcgcgcccagcagcccgcttggccacacaggcaacgaatacattcaatagggggcctcgcagaatggaaggagcggtaaagggtacaggagcactgcgcacaaggggcctgtgcaggagtgactgactgggcgggcagacggcgcaccgcgggcgcaggcaagcagggaagattgaagcggcagggaggaggatgctgattgaggggggcatcgcagtctctcttggacccgggataaggaagcaaatattcggccggttgggttgtgtgtgtgcacgttttcttcttcagagtcgtgggtgtgcttccagggaggatataagcagcaggatcgaatcccgcgaccagcgtttccccatccagccaaccaccctgtcggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacggcgcgccatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcttgttttccagaaggagttgctccttgagcctttcattctcagcctcgataacctccaaagccgctctaattgtggagggggttcgaaccgaatgctgcgtgaacgggaaggaggaggagaaagagtgagcagggagggattcagaaatgagaaatgagaggtgaaggaacgcatccctatgcccttgcaatggacagtgtttctggccaccgccaccaagacttcgtgtcctctgatcatcatgcgattgattacgttgaatgcgacggccggtcagccccggacctccacgcaccggtgctcctccaggaagatgcgcttgtcctccgccatcttgcagggctcaagctgctcccaaaactcttgggcgggttccggacggacggctaccgcgggtgcggccctgaccgccactgttcggaagcagcggcgctgcatgggcagcggccgctgcggtgcgccacggaccgcatgatccaccggaaaagcgcacgcgctggagcgcgcagaggaccacagagaagcggaagagacgccagtactggcaagcaggctggtcggtgccatggcgcgctactaccctcgctatgactcgggtcctcggccggctggcggtgctgacaattcgtttagtggagcagcgactccattcagctaccagtcgaactcagtggcacagtgactccgctcttc

SEQ ID NO: 67 - DNA, 1003 bp, Artificial Sequence
  Description of Artificial Sequence Synthetic polynucleotide
atagcgactgctaccccccgaccatgtgccgaggcagaaattatatacaagaagcagatcgcaattaggcacatcgctttgcattatccacacactattcatcgctgctgcggcaaggctgcagagtgtatttttgtggcccaggagctgagtccgaagtcgacgcgacgagcggcgcaggatccgacccctagacgagctctgtcattttccaagcacgcagctaaatgcgctgagaccgggtctaaatcatccgaaaagtgtcaaaatggccgattgggttcgcctaggacaatgcgctgcggattcgctcgagtccgctgccggccaaaaggcggtggtacaggaaggcgcacggggccaaccctgcgaagccgggggcccgaacgccgaccgccggccttcgatctcgggtgtccccctcgtcaatttcctctctcgggtgcagccacgaaagtcgtgacgcaggtcacgaaatccggttacgaaaaacgcaggtcttcgcaaaaacgtgagggtttcgcgtctcgccctagctattcgtatcgccgggtcagacccacgtgcagaaaagcccttgaataacccgggaccgtggttaccgcgccgcctgcaccagggggcttatataagcccacaccacacctgtctcaccacgcatttctccaactcgcgacttttcggaagaaattgttatccacctagtatagactgccacctgcaggaccttgtgtcttgcagtttgtattggtcccggccgtcgagctcgacagatctgggctagggttggcctggccgctcggcactcccctttagccgcgcgcatccgcgttccagaggtgcgattcggtgtgtggagcattgtcatgcgcttgtgggggtcgttccgtgcgcggcgggtccgccatgggcgccgacctgggccctagggtttgttttcgggccaagcgagcccctctcacctcgtcgcccccccgcattccctctctcttgcagccttgcc

SEQ ID NO: 68 - DNA, 812 bp, Artificial Sequence
  Description of Artificial Sequence Synthetic polynucleotide
tgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatg

SEQ ID NO: 69 - DNA, 591 bp, Artificial Sequence
  Description of Artificial Sequence Synthetic polynucleotide
gtcctgaacaacgctttcggaggtcctcaggaagctggcatccccgcgccgtatgctcatccgcgcagccccgattcattcgcacccgttcgtcccctgtcccaccttccgctggcactccccgccacgcggtgccggcccggcgcccattgactcccatcccaatcgctacccacccacactctcaccgcgtgtccctgttcctgcttcgccgcagttccagcgctcccgcgcgccctgcctccctcccgcgcgcgggacatgtccctattctgctcataaccttgcctcacgcatcccttggtccacctgtatctccatagtacacacaggtcgcaaaaagaggtcaaaaatcagacgggtgcgagccggccagtgcccttcggtcatcccttggaagccagaacgaatcagcagggccgccccacgtgatatttgctggggatcgcgcgatgacgatgcacaagaccatccaatgaacaagccagcgggcagccacggaaaaccccccaatgccaacactctggccgttctctcactttgttcccacccactcgcccggtcgaccagcagcgcaacatggccatgatg

SEQ ID NO: 70 - DNA, 624 bp, Artificial Sequence
  Description of Artificial Sequence Synthetic polynucleotide
gaggaacccgcatggtggcagagcaatgccgaatgattgatcactgcgccacgtgccgggttatacaattggtgcaacggatggcgaggtcacggagggtgccccaaataacccccgtgccagccgtacacaagattctatcggctctgaaacattctgacgctcattaagtagcatgatgagacgcgaaaaacgacggagtcgggtggatgacaagggtggcatcggtgacacgatgctgccaaggatgcttatatctggattcgcactggtaccagcggctagctcaatgacagaacaacaggcaacgggcaccaccttgacaatcatgatgcgcaatactggcctgctttcgtacttttacttgcatgtcatccagttgagaaacgccatctcgattgattcactcagttgtgtcaccaagtatgggcctggatcacctgcctttccgcgcccttcgttagtcactgccccttcctttcttcgggcacaaacgcccggcgcccccgggcccgtgggggctccctttgagtacgcattcatccccaagacgctcccctctttcgatcagcgtgtcttcctccgcttacccgtttcccttgattgaacataggcgcagcggcg

SEQ ID NO: 71 - DNA, 529 bp, Artificial Sequence
  Description of Artificial Sequence Synthetic polynucleotide
cctggatgtgagagtctcgaagaaccattcccaagacccgtatcaggacgccacgcctattccatcaaacagacccatcgtcgggcatcaagacaataccttttgcggacatagaaatgcttacggcggtatgacatacataagccctcccccaactagcctgacaaaggcttccaaagagcgatcagcccaagcaccaaaactatccaaagcacaattcccgactctgaagcaatcagttagacgtagcacgcacatttatatatgtacacaagtcaaattggtaaaacaatcgcaacctgaccaagttcagcccgttgtgctccgtctgggccctgagcgagcgagggcagaggctcagaccaggcccagtttgtcccaggcgtgatcttcgtggcgcgacccgggcaagaggagggggccccctagaagcctcggccgccctcgcaggaataaacggcctctctgcagccgggatcgccctcttccacatttctgaaaacgctgtacgtgcgcttcaacttgaaga

SEQ ID NO: 72 - DNA, 529 bp, Artificial Sequence
  Description of Artificial Sequence Synthetic polynucleotide
cctggatgtgagagtctcgaagaaccactcccaagacccgtatcaggatgccacgccaaatccagcgaacaaacccatcatcgggcaccaagacaataccttttgccaacatagaaatgcatatggcggtatgacatacatacgccctcccctaactagcctgaccactgcttcccaagagcgatcagcccaagcaccaatactaaccaaagcacaactcccgactctgaagcaatcagttcggcgtagctcgcacattcagatatgtacacacgtcgaattggtaaaacaatcgcaacctgaccgagttcagcccgttgttctccgtctgggccctgagcgagcgagggcagaggctcagaccaggcccagtttgtcccaggcgtgatcttcgtggcgcgacccgggcaagaggagggggccccctagaagcctcggccgccctcgcaggaataaacggcctctctgcagccgggatcgccctcttccacatttctgaaaacgctgtacgtgcgcttcaacttgaaga

SEQ ID NO: 73 - DNA, 570 bp, Artificial Sequence
  Description of Artificial Sequence Synthetic polynucleotide
gcctgctcaagcgggcgctcaacatgcagagcgtcagcgagacgggctgtggcgatcgcgagacggacgaggccgcctctgccctgtttgaactgagcgtcagcgctggctaaggggagggagactcatccccaggctcgcgccagggctctgatcccgtctcgggcggtgatcggcgcgcatgactacgacccaacgacgtacgagactgatgtcggtcccgacgaggagcgccgcgaggcactcccgggccaccgaccatgtttacaccgaccgaaagcactcgctcgtatccattccgtgcgcccgcacatgcatcatcttttggtaccgacttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctccttccctgcttctctgtgatcgctgatcagcaaca

SEQ ID NO: 74 - DNA, 574 bp, Artificial Sequence
  Description of Artificial Sequence Synthetic polynucleotide
gcctgctcaagcgggcgctcaacatgcagagcgtcagcgagacgggctgtggagatcgggagacggacgaggccgcctctgccctgtttgaactgagcgtcagcgctggctaaggggagggagactcatccccacgcccgcgccagggctctgatcccgtctcgggcggtgatcggcgcgcatgactacgacccaacgacgtacgagactgatgtcggtcccgacgaggagcgccgcgaggcactcccgggccaccgaccatgtttacaccgaccgaaagcactcgctcgtatccattccgtgcgcccgcacatgcatcatcttttggtaccgacttcggtcttgttttacccctacgacctgccttttccaaggtgtgagcaactcgcccgggacatgaccgaggatggatcatccggatccccaggccccagcagcccctgccagaatggctcgcgctttccagcctgcaggcccgtctcccaggtcgacgcaacctacatgaccaccccaatctgtcccagaccccaaacaccctccttccctgcttctctgtgatcgctgatcagcaaca

SEQ ID NO: 75 - DNA, 377 bp, Artificial Sequence
  Description of Artificial Sequence Synthetic polynucleotide
gcgccgagtttgcgcgatcgaatacgataaccaataaaagcacgctctaagcaaaaactagcgatgcattgtttatagtcagctgcatgaatgtagacagcctgggcaatcatgtgtcgggtgatcggcgggcaccggctcccgataacatcagggcgctcgatcgagcgtgctccgctgcagaccccatctcccctcactctcgctcgggcgaggacccggcctgcacgaccagtctgtgcagaaccgcggtcttgcaaatcctattgcgagagccaggtgccgtataggtcaagggtggtccgtttttcgctagccagcgccggtgttggcacgactatcccaccagcccgggcgcacggaggcaggccagcagg

SEQ ID NO: 76 - DNA, 573 bp, Artificial Sequence
  Description of Artificial Sequence Synthetic polynucleotide
gagtgcggaggggccggccgaccttttgatgccgcaaccacacatacgtgttgttatagtctagtagtacagtactgcaagcaccaacttgaacctcaagatggtccgtcgacccagctccagtttgcaacgaaggtcgggcgggtattggagatccagatcaaagcgtaaatgcgaccctctcccgaagagacttcatgcgtgtgtcctgaagtgcatgaaaacattccaggcagcgactcgtgctccaggctggcgttctttgcgacttgttggcccgcttcgcagtcggacctaggggcctgattccgcggtcgcgttgatgacacagaaaccaacggacgacccatgtgacaccggggactgaatcacagctgcccccaggggctagggcattcgagctgatacattgataacgctagacgaagtgcactgcggcggtaaaaagctctatttgtgccatcacagcgccttgcgtggcttcaggagcgcttgacgcgctgcatttctgaagtcgaaagccctagtcgccaggaggagggtcgactcgcccgcagttcgggaacgtttgga

SEQ ID NO: 77 - DNA, 569 bp, Artificial Sequence
  Description of Artificial Sequence Synthetic polynucleotide
gagtgcgcagggcccggccgaccctttgatgccgcaaccacacatacgtgtttttagagtctagtaatacagtactgcaagcaccaacttgaacctcaagatggtccgtcgacccagctccagtttgcaacgaaggtcgggcaggtattggagatccagatcaaagctgacatgcgaccctcccgaagagacttcatgcgtgtgtcctgaagtgcatgaaaacattccaggcagcgactcgtgctccaggctggcgtactttgcgacttgttggcccgcttcgcggtcgaacctgggggcctgattccggtcgcgttgatgacacagaaaccaacggacgacccatgtgacaccggggactgaatcacagctgcccccaggggctagggcattcgggctgatacattgataacgccagacgaagtgcacggcggcggtaaaaagctctatttgtgccatcacagcgccttgcgtggcttcaggagcgcttgacgcgctgcatttttgaagtccaaagccctagtcgccaggaggagggtcgactcgcccgcagctcgggaacgtttgga

SEQ ID NO: 78 - DNA, 1164 bp, Artificial Sequence
  Description of Artificial Sequence Synthetic polynucleotide
gatagtttatattttcgtggtcgaagcgggtggggaagggtgcgtagggtttggcaagtatgaggcatgtgtgcccagcgttgcacccaggcgggggttcatggccgacaggacgcgtgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcggggctggcgggaaaacaggcttcgtgtgctcaggttatgggaggtgcaggacagctcattaaacgccaacaatcgcacaattcatggcaagctaatcagttatttcccattaacgagctataattgtcccaaaattctggtctaccgggggtgatccttcgtgtacgggcccttccctcaaccctaggtatgcgcacatgcggtcgccgcgcaacgcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggtgtaggatttcgtaatcattcgtcctgatggggagctaccgactgccctagtatcagcccgactgcctgacgccagcgtccacttttgtgcacacattccattcgtgcccaagacatttcattgtggtgcgaagcgtccccagttacgctcacctgatccccaacctccttattgttctgtcgacagagtgggcccagaggccggtcgca

SEQ ID NO: 79 - DNA, 1173 bp, Artificial Sequence
  Description of Artificial Sequence Synthetic polynucleotide
atggtttacatccttgtggttgaggcatctggggaagggggcgtggggtttggcgagtatgaggcgtgtgtgcccagcgctgcacccaggcggggggtcatggccgacaggacgcgcgtcaaaggtgctgggcgtgtatgccctggtcggcaggtcgttgctgttgctgcgctcgtggttccgcaaccctgattttggcgtcttattctggcgtggcaagcgctgacgcccgcgagccgggccggcggcgatgcggtgtctcacggctgccgagctccaagggaggcaagagcgcccggatcagctgaagggctttacacgcaaggtacagccgctcctgcaaggctgcgtggtggacttgaacctgtaggtcctctgctgaagttcctccactacctcaccaggcccagcagaccaaagcacaggcttttcaggtccgtgtcatccactctaaaacactcgactacgacctactgatggccctagattcttcatcaacaatgcctgagacacttgctcagaattgaaactccctgaagggaccaccagaggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgcggcgcctttcttgcgataatttatgcaatggactgctctgcaaatttctgggtctgtcgccaaccctaggatcagcggcgtaggatttcgtaatcattcgtcctgatggggagctaccgactaccctaatatcagcccggctgcctgacgccagcgtccacttttgcgtacacattccattcgtgcccaagacatttcattgtggtgcgaagcgtccccagttacgctcacctgtttcccgacctccttactgttctgtcgacagagcgggcccacaggccggtcgca

SEQ ID NO: 80 - DNA, 9619 bp, Artificial Sequence
  Description of Artificial Sequence Synthetic polynucleotide
gctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggcccactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagaattcgcctgctcaagcgggcgctcaacatgcagagcgtcagcgagacgggctgtggcgatcgcgagacggacgaggccgcctctgccctgtttgaactgagcgtcagcgctggctaaggggagggagactcatccccaggctcgcgccagggctctgatcccgtctcgggcggtgatcggcgcgcatgactacgacccaacgacgtacgagactgatgtcggtcccgacgaggagcgccgcgaggcactcccgggccaccgaccatgtttacaccgaccgaaagcactcgctcgtatccattccgtgcgcccgcacatgcatcatcttttggtaccgacttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctccttccctgcttctctgtgatcgctgatcagcaacaactagtatggccaccgcatccactttctcggcgttcaatgcccgctgcggcgacctgcgtcgctcggcgggctccgggccccggcgcccagcgaggcccctccccgtgcgcgggcgcgccggtgccgtggccgctcctggccgacgcgctgcctctcgtcctctggtggtgcacgccgtggcctccgaggctcctctgggcgtgcctccctccgtgcagcgcccttctcccgtggtgtactccaagctggacaagcagcaccgcctgacgcctgagcgcctggagctggtgcagtccatgggccagttcgccgaggagcgcgtgctgcccgtgctgcaccccgtggacaagctgtggcagccccaggacttcctgcccgaccccgagtcccccgacttcgaggaccaggtggccgagctgcgcgcccgcgccaaggacctgcccgacgagtacttcgtggtgctggtgggcgacatgatcaccgaggaggccctgcccacctacatggccatgctgaacaccctggacggcgtgcgcgacgacaccggcgccgccgaccacccctgggcccgctggacccgccagtgggtggccgaggagaaccgccacggcgacctgctgaacaagtactgctggctgaccggccgcgtgaacatgcgcgccgtggaggtgaccatcaacaacctgatcaagtccggcatgaacccccagaccgacaacaacccctacctgggcttcgtgtacacctccttccaggagcgcgccaccaagtactcccacggcaacaccgcccgcctggccgccgagcacggcgacaagggcctgtccaagatctgcggcctgatcgcctccgacgagggccgccacgagatcgcctacacccgcatcgtggacgagttcttccgcctggaccccgagggcgccgtggccgcctacgccaacatgatgcgcaagcagatcaccatgcccgcccacctgatggacgacatgggccacggcgaggccaaccccggccgcaacctgttcgccgacttctccgccgtggccgagaagatcgacgtgtacgacgccgaggactactgccgcatcctggagcacctgaacgcccgctggaaggtggacgagcgccaggtgtccggccaggccgccgccgaccaggagtacgtgctgggcctgccccagcgcttccgcaagctggccgagaagaccgccgccaagcgcaagcgcgtggcccgccgccccgtggccttctcctggatctccggccgcgagatcatggtgtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagc

SEQ ID NO: 81 - DNA, 566 bp, Protheca moriformis
gtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgaca

SEQ ID NO: 82 - protein, 281 aa, Limnanthes douglasii
MAKTRTSSLRNRRQLKPAVAATADDDKDGVFMVLLSCFKIFVCFAIVLITAVAWGLIMVLLLPWPYMRIRLGNLYGHIIGGLVIWIYGIPIKIQGSEHTKKRAIYISNHASPIDAFFVMWLAPIGTVGVAKKEVIWYPLLGQLYTLAHHIRIDRSNPAAAIQSMKEAVRVITEKNLSLIMFPEGTRSRDGRLLPFKKGFVHLALQSHLPIVPMILTGTHLAWRKGTFRVRPVPITVKYLPPINTDDWTVDKIDDYVKMIHDVYVRNLPASQKPLGSTNRSN

SEQ ID NO: 83 - protein, 281 aa, Limnanthes alba
MAKTRTSSLRNRRQLKTAVAATADDDKDGIFMVLLSCFKIFVCFAIVLITAVAWGLIMVLLLPWPYMRIRLGNLYGHIIGGLVIWLYGIPIEIQGSEHTKKRAIYISNHASPIDAFFVMWLAPIGTVGVAKKEVIWYPLLGQLYTLAHHIRIDRSNPAAAIQSMKEAVRVITEKNLSLIMFPEGTRSGDGRLLPFKKGFVHLALQSHLPIVPMILTGTHLAWRKGTFRVRPVPITVKYLPPINTDDWTVDKIDDYVKMIHDIYVRNLPASQKPLGSTNRSK

SEQ ID NO: 84 - protein, 506 aa, Crambe abyssinica
MTSINVKLLYHYVITNLFNLCFFPLTAIVAGKASRLTIDDLHHLYYSYLQHNVITIAPLFAFTVFGSILYIVTRPKPVYLVEYSCYLPPTQCRSSISKVMDIFYQVRKADPFRNGTCDDSSWLDFLRKIQERSGLGDETHGPEGLLQVPPRKTFAAAREETEQVIVGALKNLFENTKVNPKDIGILVVNSSMFNPTPSLSAMVVNTFKLRSNVRSFNLGGMGCSAGVIAIDLAKDLLHVHKNTYALVVSTENITYNIYAGDNRSMMVSNCLFRVGGAAILLSNKPRDRRRSKYELVHTVRTHTGADDKSFRCVQQGDDENGKTGVSLSKDITEVAGRTVKKNIATLGPLILPLSEKLLFFVTFMAKKLFKDKVKHYYVPDFKLAIDHFCIHAGGRAVIDVLEKNLGLAPIDVEASRSTLHRFGNTSSSSIWYELAYIEAKGRMKKGNKVWQIALGSGFKCNSAVWVALSNVKASTNSPWEHCIDRYPVKIDSDSAKSETRAQNGRS

SEQ ID NO: 85 - protein, 505 aa, Lunaria annua
MTSINVKLLYHYVITNFFNLCFFPLTAILAGKASRLTTNDLHHFYSYLQHNLITLTLLFAFTVFGSVLYFVTRPKPVYLVDYSCYLPPQHLSAGISKTMEIFYQIRKSDPLRNVALDDSSSLDFLRKIQERSGLGDETYGPEGLFEIPPRKNLASAREETEQVINGALKNLFENTKVNPKEIGILVVNSSMFNPTPSLSAMVVNTFKLRSNIKSFNLGGMGCSAGVIAIDLAKDLLHVHKNTYALVVSTENITQNIYTGDNRSMMVSNCLFRVGGAAILLSNKPGDRRRSKYRLAHTVRTHTGADDKSFGCVRQEEDDSGKTGVSLSKDITGVAGITVQKNITTLGPLVLPLSEKILFVVTFVAKKLLKDKIKHYYVPDFKLAVDHFCIHAGGRAVIDVLEKNLGLSPIDVEASRSTLHRFGNTSSSSIWYELAYIEAKGRMKKGNKAWQIAVGSGFKCNSAVWVALRNVKASANSPWEHCIHKYPVQMYSGSSKSETRAQNGRS

SEQ ID NO: 86 - protein, 462 aa, Arabidopsis thaliana
MDMSSMAGSIGVSVAVLRFLLCFVATIPVSFACRIVPSRLGKHLYAAASGAFLSYLSFGFSSNLHFLVPMTIGYASMAIYRPKCGIITFFLGFAYLIGCHVFYMSGDAWKEGGIDSTGALMVLTLKVISCSMNYNDGMLKEEGLREAQKKNRLIQMPSLIEYFGYCLCCGSHFAGPVYEMKDYLEWTEGKGIWDTTEKRKKPSPYGATIRAILQAAICMALYLYLVPQYPLTRFTEPVYQEWGFLRKFSYQYMAGFTARWKYYFIWSISEASIIISGLGFSGWTDDASPKPKWDRAKNVDILGVELAKSAVQIPLVWNIQVSTWLRHYVYERLVQNGKKAGFFQLLATQTVSAVWHGLYPGYMMFFVQSALMIAGSRVIYRWQQAISPKMAMLRNIMVFINFLYTVLVLNYSAVGFMVLSLHETLTAYGSVYYIGTIIPVGLILLSYVVPAKPSRPKPRKEE

SEQ ID NO: 87 - protein, 465 aa, Arabidopsis thaliana
MELLDMNSMAASIGVSVAVLRFLLCFVATIPISFLWRFIPSRLGKHIYSAASGAFLSYLSFGFSSNLHFLVPMTIGYASMAIYRPLSGFITFFLGFAYLIGCHVFYMSGDAWKEGGIDSTGALMVLTLKVISCSINYNDGMLKEEGLREAQKKNRLIQMPSLIEYFGYCLCCGSHFAGPVFEMKDYLEWTEEKGIWAVSEKGKRPSPYGAMIRAVFQAAICMALYLYLVPQFPLTRFTEPVYQEWGFLKRFGYQYMAGFTARWKYYFIWSISEASIIISGLGFSGWTDETQTKAKWDRAKNVDILGVELAKSAVQIPLFWNIQVSTWLRHYVYERIVKPGKKAGFFQLLATQTVSAVWHGLYPGYIIFFVQSALMIDGSKAIYRWQQAIPPKMAMLRNVLVLINFLYTVVVLNYSSVGFMVLSLHETLVAFKSVYYIGTVIPIAVLLLSYLVPVKPVRPKTRKEE

SEQ ID NO: 88 - protein, 466 aa, Brassica rapa
MISMDMDSMAASIGVSVAVLRFLLCFVATIPVSFFWRIVPSRLGKHVYAAASGVFLSYLSFGFSSNLHFLVPMTIGYASMAMYRPKCGIITFFLGFAYLIGCHVFYMSGDAWKEGGIDSTGALMVLTLKVISCAVNYNDGMLKEEGLREAQKKNRLIEMPSLIEYFGYCLCCGSHFAGPVYEMKDYLQWTEGTGIWDSSEKRKQPSPYLATLRAIFQAGICMALYLYLVPQFPLTRFTEPVYQEWGFWKKFGYQYMAGQTARWKYYFIWSISEASIIISGLGFSGWTDDEASPKPKWDRAKNVDILGVELAKSAVQIPLVWNIQVSTWLRHYVYERLVKSGKKAGFFQLLATQTVSAVWHGLYPGYMMFFVQSALMIAGSRVIYRWQQAISPKLGVLRSMMVFINFLYTVLVLNYSAVGFMVLSLHETLTAYGSVYYIGTIIPVGLILLSYVVPAKPYRAKPRKEE

SEQ ID NO: 89 - protein, 466 aa, Brassica juncea
MISMDMDSMAASIGVSVAVLRFLLCFVATIPVSFFWRIVPSRLGKHIYAAASGVFLSYLSFGFSSNLHFLVPMTIGYASMAMYRPKCGIITFFLGFAYLIGCHVFYMSGDAWKEGGIDSTGALMVLTLKVISCAVNYNDGMLKEEGLREAQKKNRLIEMPSLIEYFGYCLCCGSHFAGPVYEMKDYLQWTEGTGIWDSSEKRKQPSPYLATLRAIFQAGICMALYLYLVPQFPLTRFTEPVYQEWGFWKKFGYQYMAGQTARWKYYFIWSISEASIIISGLGFSGWTDDDASPKPKWDRAKNVDILGVELAKSAVQIPLVWNIQVSTWLRHYVYERLVKSGKKAGFFQLLATQTVSAVWHGLYPGYMMFFVQSALMIAGSRVIYRWQQAISPKLGVLRSMMVFINFLYTVLVLNYSAVGFMVLSLHETLTAYGSVYYIGTIIPVGLILLSYVVPAKPYRAKPRKEE

SEQ ID NO: 90 - protein, 466 aa, Brassica juncea
MISMDMNSMAASIGVSVAVLRFLLCFVATIPVSFAWRIVPSRLGKHIYAAASGVFLSYLSFGFSSNLHFLVPMTIGYASMAMYRPKCGIITFFLGFAYLIGCHVFYMSGDAWKEGGIDSTGALMVLTLKVISCAVNYNDGMLKEEGLREAQKKNRLIQMPSLIEYFGYCLCCGSHFAGPVYEMKDYLQWTEGKGIWDSSEKRKQPSPYGATLRAIFQAGICMALYLYLVPQFPLTRFTEPVYQEWGFLKKFGYQYMAGQTARWKYYFIWSISEASIIISGLGFSGWTDDDASPKPKWDRAKNVDILGVELAKSAVQIPLVWNIQVSTWLRHYVYERLVKSGKKAGFFQLLATQTVSAVWHGLYPGYMMFFVQSALMIAGSRVIYRWQQAISPKLAMLRNIMVFINFLYTVLVLNYSAVGFMVLSLHETLTAYGSVYYIGTIIPVGLILLSYVVPAKPSRPKPRKEE

SEQ ID NO: 91 - protein, 464 aa, Limnanthes douglasii
MDLDMDSMASSIGVSVPVLRFLLCYAATIPVSFICRFVPGKTPKNVFSAATGAFLSYLSFGFSSNIHFLIPMTLGYASMALYRAKCGIVTFFLAFGYLIGCHVYYMSGDAWKEGGIDATGALMVLTLKVISCSVNYNDGLLKEEGLRPSQKKNRLSSLPSFIEYVGYCLCCGTHFAGPVYEMKDYLEWTAGKGIWAKSEKAKSPSPFLPALRALLQGAVCMVLYLYLVPQYPLSQFTSPVYQEWGFWKRLSYQYMAGFTARWKYYFIWSISEASVILSGLGFSGWTDSSPPKPRWDRAKNVDILGVEFATSGAQVPLVWNIQVSTWLRHYVYDRLVKTGKKPGFFQLLATQTTSAVWHGLYPGYLFFFVQSALMIAGSKVIYRWKQALPPSASVLQKILVFANFLYTLLVLNYSCVGFMVLSMHETIAAYGSVYYVGTIVPIVLTILGSIIPVKPRRTKVQKEQ

SEQ ID NO: 92 - protein, 460 aa, Limnanthes douglasii
MNMQNAALLIGVSVPVFRFLVSFLATVPVSFLWRYAPGNLGKHVYAAGSGALLSCLAFGLLSNLHFLVLMVMGYCSMVFYRSKCGILTFVLGFTYLIGCHFYYMSGDAWKDGGMDATGSLMVLTLKVISCAINYNDGLLKEEGLREAQKKNRLINLPSVVEYVGYCLCCGSHFAGPVFEMKDYLQWTKKKGIWAAKERSPSPYVATIRALLQAAICMVVYMYLVPRFPLSTLAEPIYQEWGFWKKLSYQYITGFSSRWKYFFVWSISEASMIISGLGFSGWTDTSPQNPQWDRAKNVDILRAELPESAVVLPLVWNIHVSTWLRHYVYERLIKNGKKPGFFELLATQTVSAVWHGLYPGYIIFFVHTALMIAGSRVIYRWRQAVPPNMALVKKMLTFMNLLYTVLILNYSYVGFRVLNLHETLAAHRSVYYVGTILPIIFIFLGYIFPAKPSRPKPRKQQ

SEQ ID NO: 93 - DNA, 6138 bp, Artificial Sequence
  Description of Artificial Sequence Synthetic polynucleotide
gctcttctgcttcggattccactacatcaagtgggtgaacctggcgggcgcggaggagggcccccgcccgggcggcattgttagcaaccactgcagctacctggacatcctgctgcacatgtccgattccttccccgcctttgtggcgcgccagtcgacggccaagctgccctttatcggcatcatcaggtgcgtgaaagtgggggctgctgtggtcgtggtgggcggggtcacaaatgaggacattgatgctgtcgtttgccgatcaggggagctcgaaagtaagtgcagcctggtcatgggatcacaaatctcaccaccactcgtccaccttgcctgggccttgcagccaaattatgagctgcctctacgtgaaccgcgaccgctcggggcccaaccacgtgggtgtggccgacctggtgaagcagcgcatgcaggacgaggccgaggggaagaccccgcccgagtaccggccgctgctcctcttccccgaggtgggcttttgagacactgtttgtgcttgaaactgtggacgcgcgtgccctgacgcgcctccggcgcctgtctcgcatccattcgcctctcaaccccatctcaccttttctccatcgccagggcaccacctccaacggcgactacctgcttcccttcaagaccggcgccttcctggccggggtgcccgtccagcccgtggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatgtccgccgccgccgccgagaccgacgtgtccctgcgccgccgctccaactccctgaacggcaaccacaccaacggcgtggccatcgacggcaccctggacaacaacaaccgccgcgtgggcgacaccaacacccacatggacatctccgccaagaagaccgacaacggctacgccaacggcgtgggcggcggcggctggcgctccaaggcctccttcaccacctggaccgcccgcgacatcgtgtacgtggtgcgctaccactggatcccctgcatgttcgccgccggcctgctgttcttcatgggcgtggagtacaccctgcagatgatccccgcccgctccgagcccttcgacctgggcttcgtggtgacccgctccctgaaccgcgtgctggcctcctcccccgacctgaacaccgtgctggccgccctgaacaccgtgttcgtgggcatgcagaccacctacatcgtgtggacctggctggtggagggccgcgcccgcgccaccatcgccgccctgttcatgttcacctgccgcggcatcctgggctactccacccagctgcccctgccccaggacttcctgggctccggcgtggacttccccgtgggcaacgtgtccttcttcctgttcttctccggccacgtggccggctccatgatcgcctccctggacatgcgccgcatgcagcgcctgcgcctggccatggtgttcgacatcctgaacgtgctgcagtccatccgcctgctgggcacccgcggccactacaccatcgacctggccgtgggcgtgggcgccggcatcctgttcgactccctggccggcaagtacgaggagatgatgtccaagcgccacctgggcaccggcttctccctgatctccaaggactccctggtgaactgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgtcctccactaccacagggtatggtcgtgtggggtcgagcgtgttgaagcgcagaaggggatgcgccgtcaagatcaggagctaaaaatggtgccagcgaggatccagcgctctcactcttgctgccatcgctcccacccttttccccaggggaccctgtggcccacgtgggagacgattccggccaagtggcacatcttcctgatgctctgccacccccgccacaaagtgaccgtgatgaaggttaggacaagggtcgggacccgattctggatatgacctctgaggtgtgtttctcgcgcaagcgtcccccaattcgttacaccacatccctcacaccctcgcccctgacactcgcagttgcccgtgtacgtccccaatgaggaggaaaaggccgaccccaagctgtacgcccaaaacgtccgcaaagccatggtgcgtcgggaaccgtcaaagtttgcttgcgggtgggcggggcggctctagcgaattggctcattggccctcaccgaggcagcacatcggacaccagtcgccacccggcttgcatcttcgccccctttcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagc

SEQ ID NO: 94 - DNA, 6402 bp, Artificial Sequence
  Description of Artificial Sequence Synthetic polynucleotide
gctcttctgcttcggattccactacatcaagtgggtgaacctggcgggcgcggaggagggcccccgcccgggcggcattgttagcaaccactgcagctacctggacatcctgctgcacatgtccgattccttccccgcctttgtggcgcgccagtcgacggccaagctgccctttatcggcatcatcaggtgcgtgaaagtgggggctgctgtggtcgtggtgggcggggtcacaaatgaggacattgatgctgtcgtttgccgatcaggggagctcgaaagtaagtgcagcctggtcatgggatcacaaatctcaccaccactcgtccaccttgcctgggccttgcagccaaattatgagctgcctctacgtgaaccgcgaccgctcggggcccaaccacgtgggtgtggccgacctggtgaagcagcgcatgcaggacgaggccgaggggaagaccccgcccgagtaccggccgctgctcctcttccccgaggtgggcttttgagacactgtttgtgcttgaaactgtggacgcgcgtgccctgacgcgcctccggcgcctgtctcgcatccattcgcctctcaaccccatctcaccttttctccatcgccagggcaccacctccaacggcgactacctgcttcccttcaagaccggcgccttcctggccggggtgcccgtccagcccgtggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatgggctacatcggcgcccacggcgtggccgccctgcaccgctacaagtactccggcgtggaccactcctacctggccaagtacgtgctgcagcccttctggacccgcttcgtgaaggtgttccccctgtggatgccccccaacatgatcaccctgatgggcttcatgttcctggtgacctcctccctgctgggctacatctactccccccagctggactccccccccccccgctgggtgcacttcgcccacggcctgctgctgttcctgtaccagaccttcgacgccgtggacggcaagcaggcccgccgcaccaactcctcctcccccctgggcgagctgttcgaccacggctgcgacgccctggcctgcgccttcgaggccatggccttcggctccaccgccatgtgcggccgcgacaccttctggttctgggtgatctccgccatccccttctacggcgccacctgggagcactacttcaccaacaccctgatcctgcccgtgatcaacggccccaccgagggcctggccctgatcttcgtgtcccacttcttcaccgccatcgtgggcgccgagtggtgggcccagcagctgggccagtccatccccctgttctcctgggtgcccttcgtgaacgagatccagacctcccgcgccgtgctgtacatgatgatcgccttcgccgtgatccccaccgtggccttcaacgtgaccaacgtgtacaaggtggtgcgctcccgcaacggctccatggtgctggccctggccatgctgtaccccttcgtggtgctgctgggcggcgtgctgatctgggactacctgtcccccatcaacctgatcgccacctacccccacctggtggtgctgggcaccggcctggccttcggcttcctggtgggccgcatgatcctggcccacctgtgcgacgagcccaagggcctgaagaccaacatgtgcatgtccctgctgtacctgcccttcgccctggccaacgccctgaccgcccgcctgaacgccggcgtgcccctggtggacgagctgtgggtgctgctgggctactgcatcttcaccgtgtccctgtacctgcacttcgccacctccgtgatccacgagatcaccgaggccctgggcatctactgcttccgcatcacccgcaaggaggcctgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgtcctccactaccacagggtatggtcgtgtggggtcgagcgtgttgaagcgcagaaggggatgcgccgtcaagatcaggagctaaaaatggtgccagcgaggatccagcgctctcactcttgctgccatcgctcccacccttttccccaggggaccctgtggcccacgtgggagacgattccggccaagtggcacatcttcctgatgctctgccacccccgccacaaagtgaccgtgatgaaggttaggacaagggtcgggacccgattctggatatgacctctgaggtgtgtttctcgcgcaagcgtcccccaattcgttacaccacatccctcacaccctcgcccctgacactcgcagttgcccgtgtacgtccccaatgaggaggaaaaggccgaccccaagctgtacgcccaaaacgtccgcaaagccatggtgcgtcgggaaccgtcaaagtttgcttgcgggtgggcggggcggctctagcgaattggctcattggccctcaccgaggcagcacatcggacaccagtcgccacccggcttgcatcttcgccccctttcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagc

SEQ ID NO: 95 - DNA, 1191 bp, Artificial Sequence
  Description of Artificial Sequence Synthetic polynucleotide
actagtatgggctacatcggcgcccacggcgccgccgccctgcaccgctacaagtactccggcgaggaccactcctacctggccaagtacctgctgaaccccttctggacccgcttcgtgaaggtgttccccctgtggatgccccccaacatgatcaccctgatgggcttcatgttcctggtgacctcctccctgctgggctacatctactccccccagctggactccccccccccccgctgggtgcacttcgcccacggcctgctgctgttcctgtaccagaccttcgacgccgtggacggcaagcaggcccgccgcaccaactcctcctcccccctgggcgagctgttcgaccacggctgcgacgccctggcctgcgccttcgaggccatggccttcggctccaccgccatgtgcggccgcgacaccttctggttctgggtgatctccgccatccccttctacggcgccacctgggagcactacttcaccaacaccctgatcctgcccgtgatcaacggccccaccgagggcctggccctgatctacgtgtcccacttcttcaccgccctggtgggcgccgagtggtgggcccagcagctgggcgagtccatccccctgttctcctgggtgcccttcgtgaacgccatccagacctcccgcgccgtgctgtacatgatgatcgccttcgccgtgatccccaccgtggccatcaacgtgtccaacgtgtacaaggtggtgcagtcccgcaagggctccatggtgctggccctggccatgctgtaccccttcgtggtgctgctgggcggcgtgctgatctgggactacctgtcccccatcaacctgatcgagacctacccccacctggtggtgctgggcaccggcctggccttcggcttcctggtgggccgcatgatcctggcccacctgtgcgacgagcccaagggcctgaagaccaacatgtgcatgtccctggtgtacctgcccttcgccctggccaacgccctgaccgcccgcctgaacaacggcgtgcccctggtggacgagctgtgggtgctgctgggctactgcatcttcaccgtgtccctgtacctgcacttcgccacctccgtgatccacgagatcaccgccgccctgggcatctactgcttccgcatcaccaagaagctggagaagaagccctgacttaag

SEQ ID NO: 96 - DNA, 1191 bp, Artificial Sequence
  Description of Artificial Sequence Synthetic polynucleotide
actagtatgggctacatcggcgcccacggcgtgggcgccctgcaccgctacaagtactccggcgaggaccactcctacctggccaagtacctgctgaaccccttctggacccgcttcgtgaagatcttccccctgtggatgccccccaacatgatcaccctgatgggcttcatgttcctggtgacctcctccctgctgggctacatctactccccccagctggactccccccccccccgctgggtgcacttcgcccacggcctgctgctgttcctgtaccagaccttcgacgccgtggacggcaagcaggcccgccgcaccaactcctcctcccccctgggcgagctgttcgaccacggctgcgacgccctggcctgcgccttcgaggccatggccttcggctccaccgccatgtgcggccgcgacaccttctggttctgggtgatctccgccatccccttctacggcgccacctgggagcactacttcaccaacaccctgatcctgcccgtgatcaacggccccaccgagggcctggccctgatctacgtgtcccacttcttcaccgccatcgtgggcgccgagtggtgggcccagcagctgggcgagtccatccccctgttctcctgggtgcccttcgtgaacgccatccagacctcccgcgccgtgctgtacatgatgatcgccttcgccgtgatccccaccgtggccttcaacgtgtccaacgtgtacaaggtggtgcagtcccgcaagggctccatggtgctggccctggccatgctgtaccccttcgtggtgctgctgggcggcgtgctgatctgggactacctgtcccccatcaacctgatcgccacctacccccacctggtggtgctgggcaccggcctggccttcggcttcctggtgggccgcatgatcctggcccacctgtgcgacgagcccaagggcctgaagaccaacatgtgcatgtccctggtgtacctgcccttcgccctggccaacgccctgaccgcccgcctgaacgccggcgtgcccctggtggacgagctgtgggtgctgctgggctactgcatcttcaccgtgtccctgtacctgcacttcgccacctccgtgatccacgagatcaccgccgccctgggcatctactgcttccgcatcaccaagaagctggagaagaagccctgacttaag

SEQ ID NO: 97 - DNA, 12759 bp, Artificial Sequence
  Description of Artificial Sequence Synthetic polynucleotide
gctcttctgcttcggattccactacatcaagtgggtgaacctggcgggcgcggaggagggcccccgcccgggcggcattgttagcaaccactgcagctacctggacatcctgctgcacatgtccgattccttccccgcctttgtggcgcgccagtcgacggccaagctgccctttatcggcatcatcaggtgcgtgaaagtgggggctgctgtggtcgtggtgggcggggtcacaaatgaggacattgatgctgtcgtttgccgatcaggggagctcgaaagtaagtgcagcctggtcatgggatcacaaatctcaccaccactcgtccaccttgcctgggccttgcagccaaattatgagctgcctctacgtgaaccgcgaccgctcggggcccaaccacgtgggtgtggccgacctggtgaagcagcgcatgcaggacgaggccgaggggaagaccccgcccgagtaccggccgctgctcctcttccccgaggtgggcttttgagacactgtttgtgcttgaaactgtggacgcgcgtgccctgacgcgcctccggcgcctgtctcgcatccattcgcctctcaaccccatctcaccttttctccatcgccagggcaccacctccaacggcgactacctgcttcccttcaagaccggcgccttcctggccggggtgcccgtccagcccgtggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggacatgtcctccatggccggctccatcggcgtgtccgtggccgtgctgcgcttcctgctgtgcttcgtggccaccatccccgtgtccttcgcctgccgcatcgtgccctcccgcctgggcaagcacctgtacgccgccgcctccggcgccttcctgtcctacctgtccttcggcttctcctccaacctgcacttcctggtgcccatgaccatcggctacgcctccatggccatctaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgctccatgaactacaacgacggcatgctgaaggaggagggcctgcgcgaggcccagaagaagaaccgcctgatccagatgccctccctgatcgagtacttcggctactgcctgtgctgcggctcccacttcgccggccccgtgtacgagatgaaggactacctggagtggaccgagggcaagggcatctgggacaccaccgagaagcgcaagaagccctccccctacggcgccaccatccgcgccatcctgcaggccgccatctgcatggccctgtacctgtacctggtgccccagtaccccctgacccgcttcaccgagcccgtgtaccaggagtggggcttcctgcgcaagttctcctaccagtacatggccggcttcaccgcccgctggaagtactacttcatctggtccatctccgaggcctccatcatcatctccggcctgggcttctccggctggaccgacgacgcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgcagaacggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagatggccatgctgcgcaacatcatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctcccgccccaagccccgcaaggaggagtgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgtcctccactaccacagggtatggtcgtgtggggtcgagcgtgttgaagcgcagaaggggatgcgccgtcaagatcaggagctaaaaatggtgccagcgaggatccagcgctctcactcttgctgccatcgctcccacccttttccccaggggaccctgtggcccacgtgggagacgattccggccaagtggcacatcttcctgatgctctgccacccccgccacaaagtgaccgtgatgaaggttaggacaagggtcgggacccgattctggatatgacctctgaggtgtgtttctcgcgcaagcgtcccccaattcgttacaccacatccctcacaccctcgcccctgacactcgcagttgcccgtgtacgtccccaatgaggaggaaaaggccgaccccaagctgtacgcccaaaacgtccgcaaagccatggtgcgtcgggaaccgtcaaagtttgcttgcgggtgggcggggcggctctagcgaattggctcattggccctcaccgaggcagcacatcggacaccagtcgccacccggcttgcatcttcgccccctttcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagcgctcttctgcttcggattccactacatcaagtgggtgaacctggcgggcgcggaggagggcccccgcccgggcggcattgttagcaaccactgcagctacctggacatcctgctgcacatgtccgattccttccccgcctttgtggcgcgccagtcgacggccaagctgccctttatcggcatcatcaggtgcgtgaaagtgggggctgctgtggtcgtggtgggcggggtcacaaatgaggacattgatgctgtcgtttgccgatcaggggagctcgaaagtaagtgcagcctggtcatgggatcacaaatctcaccaccactcgtccaccttgcctgggccttgcagccaaattatgagctgcctctacgtgaaccgcgaccgctcggggcccaaccacgtgggtgtggccgacctggtgaagcagcgcatgcaggacgaggccgaggggaagaccccgcccgagtaccggccgctgctcctcttccccgaggtgggcttttgagacactgtttgtgcttgaaactgtggacgcgcgtgccctgacgcgcctccggcgcctgtctcgcatccattcgcctctcaaccccatctcaccttttctccatcgccagggcaccacctccaacggcgactacctgcttcccttcaagaccggcgccttcctggccggggtgcccgtccagcccgtggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatgtccgccgccgccgccgagaccgacgtgtccctgcgccgccgctccaactccctgaacggcaaccacaccaacggcgtggccatcgacggcaccctggacaacaacaaccgccgcgtgggcgacaccaacacccacatggacatctccgccaagaagaccgacaacggctacgccaacggcgtgggcggcggcggctggcgctccaaggcctccttcaccacctggaccgcccgcgacatcgtgtacgtggtgcgctaccactggatcccctgcatgttcgccgccggcctgctgttcttcatgggcgtggagtacaccctgcagatgatccccgcccgctccgagcccttcgacctgggcttcgtggtgacccgctccctgaaccgcgtgctggcctcctcccccgacctgaacaccgtgctggccgccctgaacaccgtgttcgtgggcatgcagaccacctacatcgtgtggacctggctggtggagggccgcgcccgcgccaccatcgccgccctgttcatgttcacctgccgcggcatcctgggctactccacccagctgcccctgccccaggacttcctgggctccggcgtggacttccccgtgggcaacgtgtccttcttcctgttcttctccggccacgtggccggctccatgatcgcctccctggacatgcgccgcatgcagcgcctgcgcctggccatggtgttcgacatcctgaacgtgctgcagtccatccgcctgctgggcacccgcggccactacaccatcgacctggccgtgggcgtgggcgccggcatcctgttcgactccctggccggcaagtacgaggagatgatgtccaagcgccacctgggcaccggcttctccctgatctccaaggactccctggtgaactgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgtcctccactaccacagggtatggtcgtgtggggtcgagcgtgttgaagcgcagaaggggatgcgccgtcaagatcaggagctaaaaatggtgccagcgaggatccagcgctctcactcttgctgccatcgctcccacccttttccccaggggaccctgtggcccacgtgggagacgattccggccaagtggcacatcttcctgatgctctgccacccccgccacaaagtgaccgtgatgaaggttaggacaagggtcgggacccgattctggatatgacctctgaggtgtgtttctcgcgcaagcgtcccccaattcgttacaccacatccctcacaccctcgcccctgacactcgcagttgcccgtgtacgtccccaatgaggaggaaaaggccgaccccaagctgtacgcccaaaacgtccgcaaagccatggtgcgtcgggaaccgtcaaagtttgcttgcgggtgggcggggcggctctagcgaattggctcattggccctcaccgaggcagcacatcggacaccagtcgccacccggcttgcatcttcgccccctttcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagc

SEQ ID NO: 98 - DNA, 1398 bp, Arabidopsis thaliana
atggagctgctggacatgaactccatggccgcctccatcggcgtgtccgtggccgtgctgcgcttcctgctgtgcttcgtggccaccatccccatctccttcctgtggcgcttcatcccctcccgcctgggcaagcacatctactccgccgcctccggcgccttcctgtcctacctgtccttcggcttctcctccaacctgcacttcctggtgcccatgaccatcggctacgcctccatggccatctaccgccccctgtccggcttcatcaccttcttcctgggcttcgcctacctgatcggctgccacgtgttctacatgtccggcgacgcctggaaggagggcggcatcgactccaccggcgccctgatggtgctgaccctgaaggtgatctcctgctccatcaactacaacgacggcatgctgaaggaggagggcctgcgcgaggcccagaagaagaaccgcctgatccagatgccctccctgatcgagtacttcggctactgcctgtgctgcggctcccacttcgccggccccgtgttcgagatgaaggactacctggagtggaccgaggagaagggcatctgggccgtgtccgagaagggcaagcgcccctccccctacggcgccatgatccgcgccgtgttccaggccgccatctgcatggccctgtacctgtacctggtgccccagttccccctgacccgcttcaccgagcccgtgtaccaggagtggggcttcctgaagcgcttcggctaccagtacatggccggcttcaccgcccgctggaagtactacttcatctggtccatctccgaggcctccatcatcatctccggcctgggcttctccggctggaccgacgagacccagaccaaggccaagtgggaccgcgccaagaacgtggacatcctgggcgtggagctggccaagtccgccgtgcagatccccctgttctggaacatccaggtgtccacctggctgcgccactacgtgtacgagcgcatcgtgaagcccggcaagaaggccggcttcttccagctgctggccacccagaccgtgtccgccgtgtggcacggcctgtaccccggctacatcatcttcttcgtgcagtccgccctgatgatcgacggctccaaggccatctaccgctggcagcaggccatcccccccaagatggccatgctgcgcaacgtgctggtgctgatcaacttcctgtacaccgtggtggtgctgaactactcctccgtgggcttcatggtgctgtccctgcacgagaccctggtggccttcaagtccgtgtactacatcggcaccgtgatccccatcgccgtgctgctgctgtcctacctggtgcccgtgaagcccgtgcgccccaagacccgcaaggaggagtga

SEQ ID NO: 99 - DNA, 1413 bp, Brassica rapa
atcagtatgatctccatggacatggactccatggccgcctccatcggcgtgtccgtggccgtgctgcgcttcctgctgtgcttcgtggccaccatccccgtgtccttcttctggcgcatcgtgccctcccgcctgggcaagcacgtgtacgccgccgcctccggcgtgttcctgtcctacctgtccttcggcttctcctccaacctgcacttcctggtgcccatgaccatcggctacgcctccatggccatgtaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgcgccgtgaactacaacgacggcatgctgaaggaggagggcctgcgcgaggcccagaagaagaaccgcctgatcgagatgccctccctgatcgagtacttcggctactgcctgtgctgcggctcccacttcgccggccccgtgtacgagatgaaggactacctgcagtggaccgagggcaccggcatctgggactcctccgagaagcgcaagcagccctccccctacctggccaccctgcgcgccatcttccaggccggcatctgcatggccctgtacctgtacctggtgccccagttccccctgacccgcttcaccgagcccgtgtaccaggagtggggcttctggaagaagttcggctaccagtacatggccggccagaccgcccgctggaagtactacttcatctggtccatctccgaggcctccatcatcatctccggcctgggcttctccggctggaccgacgacgaggcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgaagtccggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagctgggcgtgctgcgctccatgatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctaccgcgccaagccccgcaaggaggagtgacttaag

SEQ ID NO: 100 - DNA, 1401 bp, Brassica juncea
atgatctccatggacatgaactccatggccgcctccatcggcgtgtccgtggccgtgctgcgcttcctgctgtgcttcgtggccaccatccccgtgtccttcgcctggcgcatcgtgccctcccgcctgggcaagcacatctacgccgccgcctccggcgtgttcctgtcctacctgtccttcggcttctcctccaacctgcacttcctggtgcccatgaccatcggctacgcctccatggccatgtaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgcgccgtgaactacaacgacggcatgctgaaggaggagggcctgcgcgaggcccagaagaagaaccgcctgatccagatgccctccctgatcgagtacttcggctactgcctgtgctgcggctcccacttcgccggccccgtgtacgagatgaaggactacctgcagtggaccgagggcaagggcatctgggactcctccgagaagcgcaagcagccctccccctacggcgccaccctgcgcgccatcttccaggccggcatctgcatggccctgtacctgtacctggtgccccagttccccctgacccgcttcaccgagcccgtgtaccaggagtggggcttcctgaagaagttcggctaccagtacatggccggccagaccgcccgctggaagtactacttcatctggtccatctccgaggcctccatcatcatctccggcctgggcttctccggctggaccgacgacgacgcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgaagtccggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagctggccatgctgcgcaacatcatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctcccgccccaagccccgcaaggaggagtga

SEQ ID NO: 101 - DNA, 1401 bp, Limnanthes douglasii
actagtatggacctggacatggactccatggcctcctccatcggcgtgtccgtgcccgtgctgcgcttcctgctgtgctacgccgccaccatccccgtgtccttcatctgccgcttcgtgcccggcaagacccccaagaacgtgttctccgccgccaccggcgccttcctgtcctacctgtccttcggcttctcctccaacatccacttcctgatccccatgaccctgggctacgcctccatggccctgtaccgcgccaagtgcggcatcgtgaccttcttcctggccttcggctacctgatcggctgccacgtgtactacatgtccggcgacgcctggaaggagggcggcatcgacgccaccggcgccctgatggtgctgaccctgaaggtgatctcctgctccgtgaactacaacgacggcctgctgaaggaggagggcctgcgcccctcccagaagaagaaccgcctgtcctccctgccctccttcatcgagtacgtgggctactgcctgtgctgcggcacccacttcgccggccccgtgtacgagatgaaggactacctggagtggaccgccggcaagggcatctgggccaagtccgagaaggccaagtccccctcccccttcctgcccgccctgcgcgccctgctgcagggcgccgtgtgcatggtgctgtacctgtacctggtgccccagtaccccctgtcccagttcacctcccccgtgtaccaggagtggggcttctggaagcgcctgtcctaccagtacatggccggcttcaccgcccgctggaagtactacttcatctggtccatctccgaggcctccgtgatcctgtccggcctgggcttctccggctggaccgactcctccccccccaagccccgctgggaccgcgccaagaacgtggacatcctgggcgtggagttcgccacctccggcgcccaggtgcccctggtgtggaacatccaggtgtccacctggctgcgccactacgtgtacgaccgcctggtgaagaccggcaagaagcccggcttcttccagctgctggccacccagaccacctccgccgtgtggcacggcctgtaccccggctacctgttcttcttcgtgcagtccgccctgatgatcgccggctccaaggtgatctaccgctggaagcaggccctgcccccctccgcctccgtgctgcagaagatcctggtgttcgccaacttcctgtacaccctgctggtgctgaactactcctgcgtgggcttcatggtgctgtccatgcacgagaccatcgccgcctacggctccgtgtactacgtgggcaccatcgtgcccatcgtgctgaccatcctgggctccatcatccccgtgaagccccgccgcaccaaggtgcagaaggagcagtga

SEQ ID NO: 102 - DNA, 1383 bp, Limnanthes douglasii
atgaacatgcagaacgccgccctgctgatcggcgtgtccgtgcccgtgttccgcttcctggtgtccttcctggccaccgtgcccgtgtccttcctgtggcgctacgcccccggcaacctgggcaagcacgtgtacgccgccggctccggcgccctgctgtcctgcctggccttcggcctgctgtccaacctgcacttcctggtgctgatggtgatgggctactgctccatggtgttctaccgctccaagtgcggcatcctgaccttcgtgctgggcttcacctacctgatcggctgccacttctactacatgtccggcgacgcctggaaggacggcggcatggacgccaccggctccctgatggtgctgaccctgaaggtgatctcctgcgccatcaactacaacgacggcctgctgaaggaggagggcctgcgcgaggcccagaagaagaaccgcctgatcaacctgccctccgtggtggagtacgtgggctactgcctgtgctgcggctcccacttcgccggccccgtgttcgagatgaaggactacctgcagtggaccaagaagaagggcatctgggccgccaaggagcgctccccctccccctacgtggccaccatccgcgccctgctgcaggccgccatctgcatggtggtgtacatgtacctggtgccccgcttccccctgtccaccctggccgagcccatctaccaggagtggggcttctggaagaagctgtcctaccagtacatcaccggcttctcctcccgctggaagtacttcttcgtgtggtccatctccgaggcctccatgatcatctccggcctgggcttctccggctggaccgacacctccccccagaacccccagtgggaccgcgccaagaacgtggacatcctgcgcgccgagctgcccgagtccgccgtggtgctgcccctggtgtggaacatccacgtgtccacctggctgcgccactacgtgtacgagcgcctgatcaagaacggcaagaagcccggcttcttcgagctgctggccacccagaccgtgtccgccgtgtggcacggcctgtaccccggctacatcatcttcttcgtgcacaccgccctgatgatcgccggctcccgcgtgatctaccgctggcgccaggccgtgccccccaacatggccctggtgaagaagatgctgaccttcatgaacctgctgtacaccgtgctgatcctgaactactcctacgtgggcttccgcgtgctgaacctgcacgagaccctggccgcccaccgctccgtgtactacgtgggcaccatcctgcccatcatcttcatcttcctgggctacatcttccccgccaagccctcccgccccaagccccgcaagcagcagtga

SEQ ID NO: 103 - DNA, 6630 bp, Artificial Sequence
  Description of Artificial Sequence Synthetic polynucleotide
gctcttctgcttcggattccactacatcaagtgggtgaacctggcgggcgcggaggagggcccccgcccgggcggcattgttagcaaccactgcagctacctggacatcctgctgcacatgtccgattccttccccgcctttgtggcgcgccagtcgacggccaagctgccctttatcggcatcatcaggtgcgtgaaagtgggggctgctgtggtcgtggtgggcggggtcacaaatgaggacattgatgctgtcgtttgccgatcaggggagctcgaaagtaagtgcagcctggtcatgggatcacaaatctcaccaccactcgtccaccttgcctgggccttgcagccaaattatgagctgcctctacgtgaaccgcgaccgctcggggcccaaccacgtgggtgtggccgacctggtgaagcagcgcatgcaggacgaggccgaggggaagaccccgcccgagtaccggccgctgctcctcttccccgaggtgggcttttgagacactgtttgtgcttgaaactgtggacgcgcgtgccctgacgcgcctccggcgcctgtctcgcatccattcgcctctcaaccccatctcaccttttctccatcgccagggcaccacctccaacggcgactacctgcttcccttcaagaccggcgccttcctggccggggtgcccgtccagcccgtggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggagctgctggacatgaactccatggccgcctccatcggcgtgtccgtggccgtgctgcgcttcctgctgtgcttcgtggccaccatccccatctccttcctgtggcgcttcatcccctcccgcctgggcaagcacatctactccgccgcctccggcgccttcctgtcctacctgtccttcggcttctcctccaacctgcacttcctggtgcccatgaccatcggctacgcctccatggccatctaccgccccctgtccggcttcatcaccttcttcctgggcttcgcctacctgatcggctgccacgtgttctacatgtccggcgacgcctggaaggagggcggcatcgactccaccggcgccctgatggtgctgaccctgaaggtgatctcctgctccatcaactacaacgacggcatgctgaaggaggagggcctgcgcgaggcccagaagaagaaccgcctgatccagatgccctccctgatcgagtacttcggctactgcctgtgctgcggctcccacttcgccggccccgtgttcgagatgaaggactacctggagtggaccgaggagaagggcatctgggccgtgtccgagaagggcaagcgcccctccccctacggcgccatgatccgcgccgtgttccaggccgccatctgcatggccctgtacctgtacctggtgccccagttccccctgacccgcttcaccgagcccgtgtaccaggagtggggcttcctgaagcgcttcggctaccagtacatggccggcttcaccgcccgctggaagtactacttcatctggtccatctccgaggcctccatcatcatctccggcctgggcttctccggctggaccgacgagacccagaccaaggccaagtgggaccgcgccaagaacgtggacatcctgggcgtggagctggccaagtccgccgtgcagatccccctgttctggaacatccaggtgtccacctggctgcgccactacgtgtacgagcgcatcgtgaagcccggcaagaaggccggcttcttccagctgctggccacccagaccgtgtccgccgtgtggcacggcctgtaccccggctacatcatcttcttcgtgcagtccgccctgatgatcgacggctccaaggccatctaccgctggcagcaggccatcccccccaagatggccatgctgcgcaacgtgctggtgctgatcaacttcctgtacaccgtggtggtgctgaactactcctccgtgggcttcatggtgctgtccctgcacgagaccctggtggccttcaagtccgtgtactacatcggcaccgtgatccccatcgccgtgctgctgctgtcctacctggtgcccgtgaagcccgtgcgccccaagacccgcaaggaggagtgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgtcctccactaccacagggtatggtcgtgtggggtcgagcgtgttgaagcgcagaaggggatgcgccgtcaagatcaggagctaaaaatggtgccagcgaggatccagcgctctcactcttgctgccatcgctcccacccttttccccaggggaccctgtggcccacgtgggagacgattccggccaagtggcacatcttcctgatgctctgccacccccgccacaaagtgaccgtgatgaaggttaggacaagggtcgggacccgattctggatatgacctctgaggtgtgtttctcgcgcaagcgtcccccaattcgttacaccacatccctcacaccctcgcccctgacactcgcagttgcccgtgtacgtccccaatgaggaggaaaaggccgaccccaagctgtacgcccaaaacgtccgcaaagccatggtgcgtcgggaaccgtcaaagtttgcttgcgggtgggcggggcggctctagcgaattggctcattggccctcaccgaggcagcacatcggacaccagtcgccacccggcttgcatcttcgccccctttcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagc

SEQ ID NO: 104 - DNA, 6078 bp, Artificial Sequence
  Description of Artificial Sequence Synthetic polynucleotide
gctcttctgcttcggattccactacatcaagtgggtgaacctggcgggcgcggaggagggcccccgcccgggcggcattgttagcaaccactgcagctacctggacatcctgctgcacatgtccgattccttccccgcctttgtggcgcgccagtcgacggccaagctgccctttatcggcatcatcaggtgcgtgaaagtgggggctgctgtggtcgtggtgggcggggtcacaaatgaggacattgatgctgtcgtttgccgatcaggggagctcgaaagtaagtgcagcctggtcatgggatcacaaatctcaccaccactcgtccaccttgcctgggccttgcagccaaattatgagctgcctctacgtgaaccgcgaccgctcggggcccaaccacgtgggtgtggccgacctggtgaagcagcgcatgcaggacgaggccgaggggaagaccccgcccgagtaccggccgctgctcctcttccccgaggtgggcttttgagacactgtttgtgcttgaaactgtggacgcgcgtgccctgacgcgcctccggcgcctgtctcgcatccattcgcctctcaaccccatctcaccttttctccatcgccagggcaccacctccaacggcgactacctgcttcccttcaagaccggcgccttcctggccggggtgcccgtccagcccgtggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaagacccgcacctcctccctgcgcaaccgccgccagctgaagcccgccgtggccgccaccgccgacgacgacaaggacggcgtgttcatggtgctgctgtcctgcttcaagatcttcgtgtgcttcgccatcgtgctgatcaccgccgtggcctggggcctgatcatggtgctgctgctgccctggccctacatgcgcatccgcctgggcaacctgtacggccacatcatcggcggcctggtgatctggatctacggcatccccatcaagatccagggctccgagcacaccaagaagcgcgccatctacatctccaaccacgcctcccccatcgacgccttcttcgtgatgtggctggcccccatcggcaccgtgggcgtggccaagaaggaggtgatctggtaccccctgctgggccagctgtacaccctggcccaccacatccgcatcgaccgctccaaccccgccgccgccatccagtccatgaaggaggccgtgcgcgtgatcaccgagaagaacctgtccctgatcatgttccccgagggcacccgctcccgcgacggccgcctgctgcccttcaagaagggcttcgtgcacctggccctgcagtcccacctgcccatcgtgcccatgatcctgaccggcacccacctggcctggcgcaagggcaccttccgcgtgcgccccgtgcccatcaccgtgaagtacctgccccccatcaacaccgacgactggaccgtggacaagatcgacgactacgtgaagatgatccacgacgtgtacgtgcgcaacctgcccgcctcccagaagcccctgggctccaccaaccgctccaactgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgtcctccactaccacagggtatggtcgtgtggggtcgagcgtgttgaagcgcagaaggggatgcgccgtcaagatcaggagctaaaaatggtgccagcgaggatccagcgctctcactcttgctgccatcgctcccacccttttccccaggggaccctgtggcccacgtgggagacgattccggccaagtggcacatcttcctgatgctctgccacccccgccacaaagtgaccgtgatgaaggttaggacaagggtcgggacccgattctggatatgacctctgaggtgtgtttctcgcgcaagcgtcccccaattcgttacaccacatccctcacaccctcgcccctgacactcgcagttgcccgtgtacgtccccaatgaggaggaaaaggccgaccccaagctgtacgcccaaaacgtccgcaaagccatggtgcgtcgggaaccgtcaaagtttgcttgcgggtgggcggggcggctctagcgaattggctcattggccctcaccgaggcagcacatcggacaccagtcgccacccggcttgcatcttcgccccctttcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagc

SEQ ID NO: 105 - DNA, 725 bp, Artificial Sequence
  Description of Artificial Sequence Synthetic polynucleotide
gctcttctgcttcggattccactacatcaagtgggtgaacctggcgggcgcggaggagggcccccgcccgggcggcattgttagcaaccactgcagctacctggacatcctgctgcacatgtccgactccttccccgcctttgtggcgcgccagtcgacggccaagctgccctttatcggcatcatcaggtgcgtgaaagcgggggctgctgtggccgtggtgggcagggttgcgaaggggggcaggcgtaggcgtgcagtgtgagcggacattgatgccgtcgtttgccggtcaggagagctcgaaatcagagccagcctggtcatgggatcacagagctcaccaccactcgtccacctcgcctgcgccttgcagccaaatcatgagctgcctctacgtgaaccgcgaccgctcggggcccaaccacgtgggcgtggccgatctggtgaagcagcgcatgcaggacgaggccgaggggaggaccccgcccgagtaccgaccgctgctcctcttccccgaggtgggctttcgaggcaccgtttgtgcttgaaactgtgggcacgcgtgccccgacgcgcctctggcgcctgcttcgcatccattcgcctctcaaccccgtctctcctttcctccatcgccagggcaccacctccaacggcgactacctgcttcccttcaagaccggcgccttcctggccggggtgcccgtccagcccgtggtacc

SEQ ID NO: 106 - DNA, 723 bp, Artificial Sequence
  Description of Artificial Sequence Synthetic polynucleotide
gagctccgtcctccactaccacagggtatggtggtgtggggtcgagcgtgttgaagcgcggaaggggatgcgctgtcaagttttggagctgaaaatggtgcccgcgaggatccagcgcgccccactcacccttgctgccatcgctccccacccttttccccagggaaccctgtggcccacgtgggagacgattccggccaagtggcacatcttcctgatgctctgccacccccgccacaaagtgaccgtgatgaaggtacgaacaagggtcgggccccgattctggatatcacgtctggggtgtgtttctcgcgcacgcgtcccccgatgcgctgcacagtctccctcacaccctcacccctaacgctcgcagttgcccgtgtacgtccccaatgaggaggaaaaggccgaccccaagctgtacgcccaaaatgttcgcaaagccatggtgcgtcgggaaccgttcaagtttgcttgcgggtgggcggggcggctctagcgaattggcgcattggccctcaccgaggcagcacatcggacaccaatcgtcacccggcgagcaattccgccccctctgtcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagc

SEQ ID NO: 107 - DNA, 858 bp, Artificial Sequence
  Description of Artificial Sequence Synthetic polynucleotide
actagtatggccaagacccgcacctcctccctgcgcaaccgccgccagctgaagaccgccgtggccgccaccgccgacgacgacaaggacggcatcttcatggtgctgctgtcctgcttcaagatcttcgtgtgcttcgccatcgtgctgatcaccgccgtggcctggggcctgatcatggtgctgctgctgccctggccctacatgcgcatccgcctgggcaacctgtacggccacatcatcggcggcctggtgatctggctgtacggcatccccatcgagatccagggctccgagcacaccaagaagcgcgccatctacatctccaaccacgcctcccccatcgacgccttcttcgtgatgtggctggcccccatcggcaccgtgggcgtggccaagaaggaggtgatctggtaccccctgctgggccagctgtacaccctggcccaccacatccgcatcgaccgctccaaccccgccgccgccatccagtccatgaaggaggccgtgcgcgtgatcaccgagaagaacctgtccctgatcatgttccccgagggcacccgctccggcgacggccgcctgctgcccttcaagaagggcttcgtgcacctggccctgcagtcccacctgcccatcgtgcccatgatcctgaccggcacccacctggcctggcgcaagggcaccttccgcgtgcgccccgtgcccatcaccgtgaagtacctgccccccatcaacaccgacgactggaccgtggacaagatcgacgactacgtgaagatgatccacgacatctacgtgcgcaacctgcccgcctcccagaagcccctgggctccaccaaccgctccaagtgacttaag

SEQ ID NO: 108 - DNA, 1413 bp, Artificial Sequence
  Description of Artificial Sequence Synthetic polynucleotide
actagtatgatctccatggacatggactccatggccgcctccatcggcgtgtccgtggccgtgctgcgcttcctgctgtgcttcgtggccaccatccccgtgtccttcttctggcgcatcgtgccctcccgcctgggcaagcacatctacgccgccgcctccggcgtgttcctgtcctacctgtccttcggcttctcctccaacctgcacttcctggtgcccatgaccatcggctacgcctccatggccatgtaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgcgccgtgaactacaacgacggcatgctgaaggaggagggcctgcgcgaggcccagaagaagaaccgcctgatcgagatgccctccctgatcgagtacttcggctactgcctgtgctgcggctcccacttcgccggccccgtgtacgagatgaaggactacctgcagtggaccgagggcaccggcatctgggactcctccgagaagcgcaagcagccctccccctacctggccaccctgcgcgccatcttccaggccggcatctgcatggccctgtacctgtacctggtgccccagttccccctgacccgcttcaccgagcccgtgtaccaggagtggggcttctggaagaagttcggctaccagtacatggccggccagaccgcccgctggaagtactacttcatctggtccatctccgaggcctccatcatcatctccggcctgggcttctccggctggaccgacgacgacgcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgaagtccggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagctgggcgtgctgcgctccatgatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctaccgcgccaagccccgcaaggaggagtgacttaag

SEQ ID NO: 109 - DNA, 1413 bp, Artificial Sequence
  Description of Artificial Sequence Synthetic polynucleotide
actagtatgatctccatggacatgaactccatggccgcctccatcggcgtgtccgtggccgtgctgcgcttcctgctgtgcttcgtggccaccatccccgtgtccttcgcctggcgcatcgtgccctcccgcctgggcaagcacatctacgccgccgcctccggcgtgttcctgtcctacctgtccttcggcttctcctccaacctgcacttcctggtgcccatgaccatcggctacgcctccatggccatgtaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgcgccgtgaactacaacgacggcatgctgaaggaggagggcctgcgcgaggcccagaagaagaaccgcctgatccagatgccctccctgatcgagtacttcggctactgcctgtgctgcggctcccacttcgccggccccgtgtacgagatgaaggactacctgcagtggaccgagggcaagggcatctgggactcctccgagaagcgcaagcagccctccccctacggcgccaccctgcgcgccatcttccaggccggcatctgcatggccctgtacctgtacctggtgccccagttccccctgacccgcttcaccgagcccgtgtaccaggagtggggcttcctgaagaagttcggctaccagtacatggccggccagaccgcccgctggaagtactacttcatctggtccatctccgaggcctccatcatcatctccggcctgggcttctccggctggaccgacgacgacgcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgaagtccggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagctggccatgctgcgcaacatcatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctcccgccccaagccccgcaaggaggagtgacttaag

SEQ ID NO: 110 - DNA, 6633 bp, Artificial Sequence
  Description of Artificial Sequence Synthetic polynucleotide
gctcttctgcttcggattccactacatcaagtgggtgaacctggcgggcgcggaggagggcccccgcccgggcggcattgttagcaaccactgcagctacctggacatcctgctgcacatgtccgattccttccccgcctttgtggcgcgccagtcgacggccaagctgccctttatcggcatcatcaggtgcgtgaaagtgggggctgctgtggtcgtggtgggcggggtcacaaatgaggacattgatgctgtcgtttgccgatcaggggagctcgaaagtaagtgcagcctggtcatgggatcacaaatctcaccaccactcgtccaccttgcctgggccttgcagccaaattatgagctgcctctacgtgaaccgcgaccgctcggggcccaaccacgtgggtgtggccgacctggtgaagcagcgcatgcaggacgaggccgaggggaagaccccgcccgagtaccggccgctgctcctcttccccgaggtgggcttttgagacactgtttgtgcttgaaactgtggacgcgcgtgccctgacgcgcctccggcgcctgtctcgcatccattcgcctctcaaccccatctcaccttttctccatcgccagggcaccacctccaacggcgactacctgcttcccttcaagaccggcgccttcctggccggggtgcccgtccagcccgtggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatgatctccatggacatgaactccatggccgcctccatcggcgtgtccgtggccgtgctgcgcttcctgctgtgcttcgtggccaccatccccgtgtccttcgcctggcgcatcgtgccctcccgcctgggcaagcacatctacgccgccgcctccggcgtgttcctgtcctacctgtccttcggcttctcctccaacctgcacttcctggtgcccatgaccatcggctacgcctccatggccatgtaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgcgccgtgaactacaacgacggcatgctgaaggaggagggcctgcgcgaggcccagaagaagaaccgcctgatccagatgccctccctgatcgagtacttcggctactgcctgtgctgcggctcccacttcgccggccccgtgtacgagatgaaggactacctgcagtggaccgagggcaagggcatctgggactcctccgagaagcgcaagcagccctccccctacggcgccaccctgcgcgccatcttccaggccggcatctgcatggccctgtacctgtacctggtgccccagttccccctgacccgcttcaccgagcccgtgtaccaggagtggggcttcctgaagaagttcggctaccagtacatggccggccagaccgcccgctggaagtactacttcatctggtccatctccgaggcctccatcatcatctccggcctgggcttctccggctggaccgacgacgacgcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgaagtccggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagctggccatgctgcgcaacatcatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctcccgccccaagccccgcaaggaggagtgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgtcctccactaccacagggtatggtcgtgtggggtcgagcgtgttgaagcgcagaaggggatgcgccgtcaagatcaggagctaaaaatggtgccagcgaggatccagcgctctcactcttgctgccatcgctcccacccttttccccaggggaccctgtggcccacgtgggagacgattccggccaagtggcacatcttcctgatgctctgccacccccgccacaaagtgaccgtgatgaaggttaggacaagggtcgggacccgattctggatatgacctctgaggtgtgtttctcgcgcaagcgtcccccaattcgttacaccacatccctcacaccctcgcccctgacactcgcagttgcccgtgtacgtccccaatgaggaggaaaaggccgaccccaagctgtacgcccaaaacgtccgcaaagccatggtgcgtcgggaaccgtcaaagtttgcttgcgggtgggcggggcggctctagcgaattggctcattggccctcaccgaggcagcacatcggacaccagtcgccacccggcttgcatcttcgccccctttcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagc

SEQ ID NO: 111 - DNA, 725 bp, Artificial Sequence; identical to SEQ ID NO: 105
  Description of Artificial Sequence Synthetic polynucleotide

SEQ ID NO: 112 - DNA, 723 bp, Artificial Sequence; identical to SEQ ID NO: 106
  Description of Artificial Sequence Synthetic polynucleotide

SEQ ID NO: 113 - DNA, 1407 bp, Artificial Sequence
  Description of Artificial Sequence Synthetic polynucleotide
actagtatggacctggacatggactccatggcctcctccatcggcgtgtccgtgcccgtgctgcgcttcctgctgtgctacgccgccaccatccccgtgtccttcatctgccgcttcgtgcccggcaagacccccaagaacgtgttctccgccgccaccggcgccttcctgtcctacctgtccttcggcttctcctccaacatccacttcctgatccccatgaccctgggctacgcctccatggccctgtaccgcgccaagtgcggcatcgtgaccttcttcctggccttcggctacctgatcggctgccacgtgtactacatgtccggcgacgcctggaaggagggcggcatcgacgccaccggcgccctgatggtgctgaccctgaaggtgatctcctgctccgtgaactacaacgacggcctgctgaaggaggagggcctgcgcccctcccagaagaagaaccgcctgtcctccctgccctccttcatcgagtacgtgggctactgcctgtgctgcggcacccacttcgccggccccgtgtacgagatgaaggactacctggagtggaccgccggcaagggcatctgggccaagtccgagaaggccaagtccccctcccccttcctgcccgccctgcgcgccctgctgcagggcgccgtgtgcatggtgctgtacctgtacctggtgccccagtaccccctgtcccagttcacctcccccgtgtaccaggagtggggcttctggaagcgcctgtcctaccagtacatggccggcttcaccgcccgctggaagtactacttcatctggtccatctccgaggcctccgtgatcctgtccggcctgggcttctccggctggaccgactcctccccccccaagccccgctgggaccgcgccaagaacgtggacatcctgggcgtggagttcgccacctccggcgcccaggtgcccctggtgtggaacatccaggtgtccacctggctgcgccactacgtgtacgaccgcctggtgaagaccggcaagaagcccggcttcttccagctgctggccacccagaccacctccgccgtgtggcacggcctgtaccccggctacctgttcttcttcgtgcagtccgccctgatgatcgccggctccaaggtgatctaccgctggaagcaggccctgcccccctccgcctccgtgctgcagaagatcctggtgttcgccaacttcctgtacaccctgctggtgctgaactactcctgcgtgggcttcatggtgctgtccatgcacgagaccatcgccgcctacggctccgtgtactacgtgggcaccatcgtgcccatcgtgctgaccatcctgggctccatcatccccgtgaagccccgccgcaccaaggtgcagaaggagcagtgacttaag

SEQ ID NO: 114 - DNA, 1395 bp, Artificial Sequence
  Description of Artificial Sequence Synthetic polynucleotide
actagtatgaacatgcagaacgccgccctgctgatcggcgtgtccgtgcccgtgttccgcttcctggtgtccttcctggccaccgtgcccgtgtccttcctgtggcgctacgcccccggcaacctgggcaagcacgtgtacgccgccggctccggcgccctgctgtcctgcctggccttcggcctgctgtccaacctgcacttcctggtgctgatggtgatgggctactgctccatggtgttctaccgctccaagtgcggcatcctgaccttcgtgctgggcttcacctacctgatcggctgccacttctactacatgtccggcgacgcctggaaggacggcggcatggacgccaccggctccctgatggtgctgaccctgaaggtgatctcctgcgccatcaactacaacgacggcctgctgaaggaggagggcctgcgcgaggcccagaagaagaaccgcctgatcaacctgccctccgtggtggagtacgtgggctactgcctgtgctgcggctcccacttcgccggccccgtgttcgagatgaaggactacctgcagtggaccaagaagaagggcatctgggccgccaaggagcgctccccctccccctacgtggccaccatccgcgccctgctgcaggccgccatctgcatggtggtgtacatgtacctggtgccccgcttccccctgtccaccctggccgagcccatctaccaggagtggggcttctggaagaagctgtcctaccagtacatcaccggcttctcctcccgctggaagtacttcttcgtgtggtccatctccgaggcctccatgatcatctccggcctgggcttctccggctggaccgacacctccccccagaacccccagtgggaccgcgccaagaacgtggacatcctgcgcgccgagctgcccgagtccgccgtggtgctgcccctggtgtggaacatccacgtgtccacctggctgcgccactacgtgtacgagcgcctgatcaagaacggcaagaagcccggcttcttcgagctgctggccacccagaccgtgtccgccgtgtggcacggcctgtaccccggctacatcatcttcttcgtgcacaccgccctgatgatcgccggctcccgcgtgatctaccgctggcgccaggccgtgccccccaacatggccctggtgaagaagatgctgaccttcatgaacctgctgtacaccgtgctgatcctgaactactcctacgtgggcttccgcgtgctgaacctgcacgagaccctggccgcccaccgctccgtgtactacgtgggcaccatcctgcccatcatcttcatcttcctgggctacatcttccccgccaagccctcccgccccaagccccgcaagcagcagtgacttaag

SEQ ID NO: 115 - DNA, 1400 bp, Artificial Sequence
  Description of Artificial Sequence Synthetic polynucleotide
actagatggacatgtcctccatggccggctccatcggcgtgtccgtggccgtgctgcgcttcctgctgtgcttcgtggccaccatccccgtgtccttcgcctgccgcatcgtgccctcccgcctgggcaagcacctgtacgccgccgcctccggcgccttcctgtcctacctgtccttcggcttctcctccaacctgcacttcctggtgcccatgaccatcggctacgcctccatggccatctaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgctccatgaactacaacgacggcatgctgaaggaggagggcctgcgcgaggcccagaagaagaaccgcctgatccagatgccctccctgatcgagtacttcggctactgcctgtgctgcggctcccacttcgccggccccgtgtacgagatgaaggactacctggagtggaccgagggcaagggcatctgggacaccaccgagaagcgcaagaagccctccccctacggcgccaccatccgcgccatcctgcaggccgccatctgcatggccctgtacctgtacctggtgccccagtaccccctgacccgcttcaccgagcccgtgtaccaggagtggggcttcctgcgcaagttctcctaccagtacatggccggcttcaccgcccgctggaagtactacttcatctggtccatctccgaggcctccatcatcatctccggcctgggcttctccggctggaccgacgacgcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgcagaacggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagatggccatgctgcgcaacatcatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctcccgccccaagccccgcaaggaggagtgacttaag

SEQ ID NO: 116 - DNA, 1410 bp, Artificial Sequence
  Description of Artificial Sequence Synthetic polynucleotide
actagtatggagctgctggacatgaactccatggccgcctccatcggcgtgtccgtggccgtgctgcgcttcctgctgtgcttcgtggccaccatccccatctccttcctgtggcgcttcatcccctcccgcctgggcaagcacatctactccgccgcctccggcgccttcctgtcctacctgtccttcggcttctcctccaacctgcacttcctggtgcccatgaccatcggctacgcctccatggccatctaccgccccctgtccggcttcatcaccttcttcctgggcttcgcctacctgatcggctgccacgtgttctacatgtccggcgacgcctggaaggagggcggcatcgactccaccggcgccctgatggtgctgaccctgaaggtgatctcctgctccatcaactacaacgacggcatgctgaaggaggagggcctgcgcgaggcccagaagaagaaccgcctgatccagatgccctccctgatcgagtacttcggctactgcctgtgctgcggctcccacttcgccggccccgtgttcgagatgaaggactacctggagtggaccgaggagaagggcatctgggccgtgtccgagaagggcaagcgcccctccccctacggcgccatgatccgcgccgtgttccaggccgccatctgcatggccctgtacctgtacctggtgccccagttccccctgacccgcttcaccgagcccgtgtaccaggagtggggcttcctgaagcgcttcggctaccagtacatggccggcttcaccgcccgctggaagtactacttcatctggtccatctccgaggcctccatcatcatctccggcctgggcttctccggctggaccgacgagacccagaccaaggccaagtgggaccgcgccaagaacgtggacatcctgggcgtggagctggccaagtccgccgtgcagatccccctgttctggaacatccaggtgtccacctggctgcgccactacgtgtacgagcgcatcgtgaagcccggcaagaaggccggcttcttccagctgctggccacccagaccgtgtccgccgtgtggcacggcctgtaccccggctacatcatcttcttcgtgcagtccgccctgatgatcgacggctccaaggccatctaccgctggcagcaggccatcccccccaagatggccatgctgcgcaacgtgctggtgctgatcaacttcctgtacaccgtggtggtgctgaactactcctccgtgggcttcatggtgctgtccctgcacgagaccctggtggccttcaagtccgtgtactacatcggcaccgtgatccccatcgccgtgctgctgctgtcctacctggtgcccgtgaagcccgtgcgccccaagacccgcaaggaggagtgacttaag

SEQ ID NO: 117 - DNA, 6621 bp, Artificial Sequence
  Description of Artificial Sequence Synthetic polynucleotide
gctcttctgcttcggattccactacatcaagtgggtgaacctggcgggcgcggaggagggcccccgcccgggcggcattgttagcaaccactgcagctacctggacatcctgctgcacatgtccgattccttccccgcctttgtggcgcgccagtcgacggccaagctgccctttatcggcatcatcaggtgcgtgaaagtgggggctgctgtggtcgtggtgggcggggtcacaaatgaggacattgatgctgtcgtttgccgatcaggggagctcgaaagtaagtgcagcctggtcatgggatcacaaatctcaccaccactcgtccaccttgcctgggccttgcagccaaattatgagctgcctctacgtgaaccgcgaccgctcggggcccaaccacgtgggtgtggccgacctggtgaagcagcgcatgcaggacgaggccgaggggaagaccccgcccgagtaccggccgctgctcctcttccccgaggtgggcttttgagacactgtttgtgcttgaaactgtggacgcgcgtgccctgacgcgcctccggcgcctgtctcgcatccattcgcctctcaaccccatctcaccttttctccatcgccagggcaccacctccaacggcgactacctgcttcccttcaagaccggcgccttcctggccggggtgcccgtccagcccgtggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggacatgtcctccatggccggctccatcggcgtgtccgtggccgtgctgcgcttcctgctgtgcttcgtggccaccatccccgtgtccttcgcctgccgcatcgtgccctcccgcctgggcaagcacctgtacgccgccgcctccggcgccttcctgtcctacctgtccttcggcttctcctccaacctgcacttcctggtgcccatgaccatcggctacgcctccatggccatctaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgctccatgaactacaacgacggcatgctgaaggaggagggcctgcgcgaggcccagaagaagaaccgcctgatccagatgccctccctgatcgagtacttcggctactgcctgtgctgcggctcccacttcgccggccccgtgtacgagatgaaggactacctggagtggaccgagggcaagggcatctgggacaccaccgagaagcgcaagaagccctccccctacggcgccaccatccgcgccatcctgcaggccgccatctgcatggccctgtacctgtacctggtgccccagtaccccctgacccgcttcaccgagcccgtgtaccaggagtggggcttcctgcgcaagttctcctaccagtacatggccggcttcaccgcccgctggaagtactacttcatctggtccatctccgaggcctccatcatcatctccggcctgggcttctccggctggaccgacgacgcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgcagaacggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagatggccatgctgcgcaacatcatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctcccgccccaagccccgcaaggaggagtgacttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgtcctccactaccacagggtatggtcgtgtggggtcgagcgtgttgaagcgcagaaggggatgcgccgtcaagatcaggagctaaaaatggtgccagcgaggatccagcgctctcactcttgctgccatcgctcccacccttttccccaggggaccctgtggcccacgtgggagacgattccggccaagtggcacatcttcctgatgctctgccacccccgccacaaagtgaccgtgatgaaggttaggacaagggtcgggacccgattctggatatgacctctgaggtgtgtttctcgcgcaagcgtcccccaattcgttacaccacatccctcacaccctcgcccctgacactcgcagttgcccgtgtacgtccccaatgaggaggaaaaggccgaccccaagctgtacgcccaaaacgtccgcaaagccatggtgcgtcgggaaccgtcaaagtttgcttgcgggtgggcggggcggctctagcgaattggctcattggccctcaccgaggcagcacatcggacaccagtcgccacccggcttgcatcttcgccccctttcttctcgcagatggaggtcgccgggaccaaggacacgacggcggtgtttgaggacaagatgcgctacctgaactccctgaagagaaagtacggcaagcctgtgcctaagaaaattgagtgaacccccgtcgtcgaccagaagagc

SEQ ID NO: 118 - DNA, 725 bp, Artificial Sequence; identical to SEQ ID NO: 105
  Description of Artificial Sequence Synthetic polynucleotide

SEQ ID NO: 119 - DNA, 723 bp, Artificial Sequence; identical to SEQ ID NO: 106
  Description of Artificial Sequence Synthetic polynucleotide

SEQ ID NO: 120 - DNA, 1410 bp, Artificial Sequence; identical to SEQ ID NO: 116
  Description of Artificial Sequence Synthetic polynucleotide

SEQ ID NO: 121 - DNA, 1413 bp, Artificial Sequence
  Description of Artificial Sequence Synthetic polynucleotide
actagtatgatctccatggacatggactccatggccgcctccatcggcgtgtccgtggccgtgctgcgcttcctgctgtgcttcgtggccaccatccccgtgtccttcttctggcgcatcgtgccctcccgcctgggcaagcacgtgtacgccgccgcctccggcgtgttcctgtcctacctgtccttcggcttctcctccaacctgcacttcctggtgcccatgaccatcggctacgcctccatggccatgtaccgccccaagtgcggcatcatcaccttcttcctgggcttcgcctacctgatcggctgccacgtgttctacatgtccggcgacgcctggaaggagggcggcatcgactccaccggcgccctgatggtgctgaccctgaaggtgatctcctgcgccgtgaactacaacgacggcatgctgaaggaggagggcctgcgcgaggcccagaagaagaaccgcctgatcgagatgccctccctgatcgagtacttcggctactgcctgtgctgcggctcccacttcgccggccccgtgtacgagatgaaggactacctgcagtggaccgagggcaccggcatctgggactcctccgagaagcgcaagcagccctccccctacctggccaccctgcgcgccatcttccaggccggcatctgcatggccctgtacctgtacctggtgccccagttccccctgacccgcttcaccgagcccgtgtaccaggagtggggcttctggaagaagttcggctaccagtacatggccggccagaccgcccgctggaagtactacttcatctggtccatctccgaggcctccatcatcatctccggcctgggcttctccggctggaccgacgacgaggcctcccccaagcccaagtgggaccgcgccaagaacgtggacatcctgggcgtggagctggccaagtccgccgtgcagatccccctggtgtggaacatccaggtgtccacctggctgcgccactacgtgtacgagcgcctggtgaagtccggcaagaaggccggcttcttccagctgctggccacccagaccgtgtccgccgtgtggcacggcctgtaccccggctacatgatgttcttcgtgcagtccgccctgatgatcgccggctcccgcgtgatctaccgctggcagcaggccatctcccccaagctgggcgtgctgcgctccatgatggtgttcatcaacttcctgtacaccgtgctggtgctgaactactccgccgtgggcttcatggtgctgtccctgcacgagaccctgaccgcctacggctccgtgtactacatcggcaccatcatccccgtgggcctgatcctgctgtcctacgtggtgcccgccaagccctaccgcgccaagccccgcaaggaggagtgacttaag

SEQ ID NO: 122 - DNA, 1413 bp, Artificial Sequence; identical to SEQ ID NO: 108
  Description of Artificial Sequence Synthetic polynucleotide

SEQ ID NO: 123 - DNA, 1413 bp, Artificial Sequence; identical to SEQ ID NO: 109
  Description of Artificial Sequence Synthetic polynucleotide

SEQ ID NO: 124 - DNA, 1407 bp, Artificial Sequence; identical to SEQ ID NO: 113
  Description of Artificial Sequence Synthetic polynucleotide

SEQ ID NO: 125 - DNA, 1395 bp, Artificial Sequence; identical to SEQ ID NO: 114
  Description of Artificial Sequence Synthetic polynucleotide

SEQ ID NO: 126 - DNA, 6138 bp, Artificial Sequence; identical to SEQ ID NO: 93
  Description of Artificial Sequence Synthetic polynucleotide

SEQ ID NO: 127 - DNA, 725 bp, Artificial Sequence; identical to SEQ ID NO: 105
  Description of Artificial Sequence Synthetic polynucleotide

SEQ ID NO: 128 - DNA, 723 bp, Artificial Sequence; identical to SEQ ID NO: 106
  Description of Artificial Sequence Synthetic polynucleotide

SEQ ID NO: 129 - DNA, 6138 bp, Artificial Sequence; identical to SEQ ID NO: 93
  Description of Artificial Sequence Synthetic polynucleotide

SEQ ID NO: 130 - DNA, 725 bp, Artificial Sequence; identical to SEQ ID NO: 105
  Description of Artificial Sequence Synthetic polynucleotide

SEQ ID NO: 131 - DNA, 723 bp, Artificial Sequence; identical to SEQ ID NO: 106
  Description of Artificial Sequence Synthetic polynucleotide

SEQ ID NO: 132 - DNA, 6402 bp, Artificial Sequence; identical to SEQ ID NO: 94
  Description of Artificial Sequence Synthetic polynucleotide

SEQ ID NO: 133 - DNA, 725 bp, Artificial Sequence; identical to SEQ ID NO: 105
  Description of Artificial Sequence Synthetic polynucleotide

SEQ ID NO: 134 - DNA, 723 bp, Artificial Sequence; identical to SEQ ID NO: 106
  Description of Artificial Sequence Synthetic polynucleotide

SEQ ID NO: 135 - DNA, 1191 bp, Artificial Sequence; identical to SEQ ID NO: 95
  Description of Artificial Sequence Synthetic polynucleotide

SEQ ID NO: 136 - DNA, 1191 bp, Artificial Sequence; identical to SEQ ID NO: 96
  Description of Artificial Sequence Synthetic polynucleotide

SEQ ID NO: 137 - DNA, 6630 bp, Artificial Sequence; identical to SEQ ID NO: 103
  Description of Artificial Sequence Synthetic polynucleotide

SEQ ID NO: 138 - DNA, 1413 bp, Artificial Sequence; identical to SEQ ID NO: 121
  Description of Artificial Sequence Synthetic polynucleotide

SEQ ID NO: 139 - DNA, 10491 bp, Artificial Sequence
  Description of Artificial Sequence Synthetic polynucleotide
gctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggcccactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagaattcgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccggtgccgtggccgctcctggccgacgcgctgcctctcgtcctctggtggtgcacgccgtggcctccgaggctcctctgggcgtgcctccctccgtgcagcgcccttctcccgtggtgtactccaagctggacaagcagcaccgcctgacgcctgagcgcctggagctggtgcagtccatgggccagttcgccgaggagcgcgtgctgcccgtgctgcaccccgtggacaagctgtggcagccccaggacttcctgcccgaccccgagtcccccgacttcgaggaccaggtggccgagctgcgcgcccgcgccaaggacctgcccgacgagtacttcgtggtgctggtgggcgacatgatcaccgaggaggccctgcccacctacatggccatgctgaacaccctggacggcgtgcgcgacgacaccggcgccgccgaccacccctgggcccgctggacccgccagtgggtggccgaggagaaccgccacggcgacctgctgaacaagtactgctggctgaccggccgcgtgaacatgcgcgccgtggaggtgaccatcaacaacctgatcaagtccggcatgaacccccagaccgacaacaacccctacctgggcttcgtgtacacctccttccaggagcgcgccaccaagtactcccacggcaacaccgcccgcctggccgccgagcacggcgacaagggcctgtccaagatctgcggcctgatcgcctccgacgagggccgccacgagatcgcctacacccgcatcgtggacgagttcttccgcctggaccccgagggcgccgtggccgcctacgccaacatgatgcgcaagcagatcaccatgcccgcccacctgatggacgacatgggccacggcgaggccaaccccggccgcaacctgttcgccgacttctccgccgtggccgagaagatcgacgtgtacgacgccgaggactactgccgcatcctggagcacctgaacgcccgctggaaggtggacgagcgccaggtgtccggccaggccgccgccgaccaggagtacgtgctgggcctgccccagcgcttccgcaagctggccgagaagaccgccgccaagcgcaagcgcgtggcccgccgccccgtggccttctcctggatctccggccgcgagatcatggtgtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagc

SEQ ID NO: 140 - DNA, 5108 bp, Artificial Sequence
  Description of Artificial Sequence Synthetic polynucleotide
gctcttcacccaactcagataataccaatacccctccttctcctcctcatccattcagtacccccccccttctcttcccaaagcagcaagcgcgtggcttacagaagaacaatcggcttccgccaaagtcgccgagcactgcccgacggcggcgcgcccagcagcccgcttggccacacaggcaacgaatacattcaatagggggcctcgcagaatggaaggagcggtaaagggtacaggagcactgcgcacaaggggcctgtgcaggagtgactgactgggcgggcagacggcgcaccgcgggcgcaggcaagcagggaagattgaagcggcagggaggaggatgctgattgaggggggcatcgcagtctctcttggacccgggataaggaagcaaatattcggccggttgggttgtgtgtgtgcacgttttcttcttcagagtcgtgggtgtgcttccagggaggatataagcagcaggatcgaatcccgcgaccagcgtttccccatccagccaaccaccctgtcggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacggcgcgccatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgaattcctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacactagtatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcaccggtgcctacgtgggtcaagtacggcatcatgtggccgctctactggttcttccaggtgtgtttgagggttttggttgcccgtattgaggtcctggtggcgcgcatggaggagaaggcgcctgtcccgctgacccccccggctaccctcccggcaccttccagggcgcgtacgggaagaaccagtagagcggccacatgatgccgtacttgacccacgtaggcaccggtgcagggtcgatgtacgtcgacgcgacgtagagcagggacatgaccgcgatgtcaaaggccaggtacatgctgctacgaagcgccgagcgctcgaaacagtgcgcggggatggccttgcgcagcgtcccgatcgtgaacggaggcttctccacaggctgcctgttcgtcttgatagccatctcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagagctcttgttttccagaaggagttgctccttgagcctttcattctcagcctcgataacctccaaagccgctctaattgtggagggggttcgaaccgaatgctgcgtgaacgggaaggaggaggagaaagagtgagcagggagggattcagaaatgagaaatgagaggtgaaggaacgcatccctatgcccttgcaatggacagtgtttctggccaccgccaccaagacttcgtgtcctctgatcatcatgcgattgattacgttgaatgcgacggccggtcagccccggacctccacgcaccggtgctcctccaggaagatgcgcttgtcctccgccatcttgcagggctcaagctgctcccaaaactcttgggcgggttccggacggacggctaccgcgggtgcggccctgaccgccactgttcggaagcagcggcgctgcatgggcagcggccgctgcggtgcgccacggaccgcatgatccaccggaaaagcgcacgcgctggagcgcgcagaggaccacagagaagcggaagagacgccagtactggcaagcaggctggtcggtgccatggcgcgctactaccctcgctatgactcgggtcctcggccggctggcggtgctgacaattcgtttagtggagcagcgactccattcagctaccagtcgaactcagtggcacagtgactccgctcttc

SEQ ID NO: 141 - DNA, 10051 bp, Artificial Sequence
  Description of Artificial Sequence Synthetic polynucleotide
gctcttcgccgccgccactcctgctcgagcgcgcccgcgcgtgcgccgccagcgccttggccttttcgccgcgctcgtgcgcgtcgctgatgtccatcaccaggtccatgaggtctgccttgcgccggctgagccactgcttcgtccgggcggccaagaggagcatgagggaggactcctggtccagggtcctgacgtggtcgcggctctgggagcgggccagcatcatctggctctgccgcaccgaggccgcctccaactggtcctccagcagccgcagtcgccgccgaccctggcagaggaagacaggtgaggggggtatgaattgtacagaacaaccacgagccttgtctaggcagaatccctaccagtcatggctttacctggatgacggcctgcgaacagctgtccagcgaccctcgctgccgccgcttctcccgcacgcttctttccagcaccgtgatggcgcgagccagcgccgcacgctggcgctgcgcttcgccgatctgaggacagtcggggaactctgatcagtctaaacccccttgcgcgttagtgttgccatcctttgcagaccggtccctccgtctctgcactctggcgcccctcctccgtctcgtggactgacggacgagagtctgggcgccgcttttctatccacaccgccctttccgcatcgaagacaccacccatcgtgccgccaggtcttccccaatcacccgccctgtggtcctctctcccagccgtgtttggtcgctgcgtccacatttttccattcgtgccccacgatcctcgcccatcttggcgccttggataggcacccttttttcagcacgccctggtgtgtagcacaacctgacctctctctaccgcatcgcctccctcccacacctcagttgactccctcgtcgcacgttgcacccgcaagctccccatttcatcctattgacaatcgcacactgtacatgtatgctcattattttgcaaaaaaacagggggtcggttcactcctggcagacgacgcggtgctgccgcgcgccgctgaggcggcgtcgcgacggcaacacccatcgcaccgcacgtcgacgagtcaacccaccctgctcaacggtgatctccccatcgcgacaccccccgtgaccgtactatgtgcgtccatacgcaacatgaaaaggaccttggtccccggaggcggcgagctcgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgccactagtatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgataccttattacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacggcgcgccatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagatatcgaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccgaggtgcacgtgcaggtgacccactccctggcccccgagaagcgcgagatcttcaactccctgaacaactgggcccaggagaacatcctggtgctgctgaaggacgtggacaagtgctggcagccctccgacttcctgcccgactccgcctccgagggcttcgacgagcaggtgatggagctgcgcaagcgctgcaaggagatccccgacgactacttcatcgtgctggtgggcgacatgatcaccgaggaggccctgcccacctaccagaccatgctgaacaccctggacggcgtgcgcgacgagaccggcgcctccctgaccccctgggccatctggacccgcgcctggaccgccgaggagaaccgccacggcgacctgctgaacaagtacctgtacctgtccggccgcgtggacatgaagcagatcgagaagaccatccagtacctgatcggctccggcatggacccccgcaccgagaacaacccctacctgggcttcatctacacctccttccaggagcgcgccaccttcatctcccacggcaacaccgcccgcctggccaaggagcacggcgacctgaagctggcccagatctgcggcatcatcgccgccgacgagaagcgccacgagaccgcctacaccaagatcgtggagaagctgttcgagatcgaccccgacggcaccgtgctggccctggccgacatgatgcgcaagaaggtgtccatgcccgcccacctgatgtacgacggccaggacgacaacctgttcgagaacttctcctccgtggcccagcgcctgggcgtgtacaccgccaaggactacgccgacatcctggagttcctggtgggccgctgggacatcgagaagctgaccggcctgtccggcgagggccgcaaggcccaggactacgtgtgcaccctgcccccccgcatccgccgcctggaggagcgcgcccagtcccgcgtgaagaaggcctccgccacccccttctcctggatcttcggccgcgagatcaacctgatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcttgttttccagaaggagttgctccttgagcctttcattctcagcctcgataacctccaaagccgctctaattgtggagggggttcgaatttaaaagcttggaatgttggttcgtgcgtctggaacaagcccagacttgttgctcactgggaaaaggaccatcagctccaaaaaacttgccgctcaaaccgcgtacctctgctttcgcgcaatctgccctgttgaaatcgccaccacattcatattgtgacgcttgagcagtctgtaattgcctcagaatgtggaatcatctgccccctgtgcgagcccatgccaggcatgtcgcgggcgaggacacccgccactcgtacagcagaccattatgctacctcacaatagttcataacagtgaccatatttctcgaagctccccaacgagcacctccatgctctgagtggccaccccccggccctggtgcttgcggagggcaggtcaaccggcatggggctaccgaaatccccgaccggatcccaccacccccgcgatgggaagaatctctccccgggatgtgggcccaccaccagcacaacctgctggcccaggcgagcgtcaaaccataccacacaaatatccttggcatcggccctgaattccttctgccgctctgctacccggtgcttctgtccgaagcaggggttgctagggatcgctccgagtccgcaaacccttgtcgcgtggcggggcttgttcgagcttgaagagc

SEQ ID NO: 142 - DNA, 7021 bp, Artificial Sequence
  Description of Artificial Sequence Synthetic polynucleotide
catatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgatgcatgctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggcccactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggaattcctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatgaaggtcacggtggtgagcaggtccggcagggaggtgctcaaggcccccctggacctgccggactccgccacggtcgctgacctccaggaggccttccacaagcgcgcgaagaagttttatcccagccgccagcggctgaccctgccggtggcccccggctccaaggacaagccggtggtgctgaactcgaagaagagcctcaaggagtactgcgacggtaacaccgactcgctcacggtggtgtttaaggacttgggcgcgcaggtctcctaccgcaccctgttcttcttcgagtacctgggccccctgctgatctaccccgtcttctactacttccctgtctataagtacctgggctacggcgaggaccgcgtcatccacccggtgcagacgtatgccatgtactactggtgcttccactactttaagcgcattatggagacgttcttcgtgcaccgcttcagccacgccacctcgcccatcggtaacgtcttccgcaactgcgcctactactggacgttcggcgcctacatcgcttactacgtgaaccaccccctgtacacccccgtgagcgacttgcagatgaagatcggcttcgggttcggcctcgtgtttcaggtggcgaacttctactgccacatcctgctgaagaatctgcgcgacccgaacggcagcggcggttaccagatcccgcgcggcttcctgttcaacatcgtcacgtgcgcgaactacaccacggagatctaccagtggctcggctttaacatcgccacgcagaccatcgccggctacgtgttcctcgcggtggccgccctgattatgaccaactgggccctcggcaagcactcgcggctccggaagatcttcgacggcaaggacggcaagccgaagtacccccgccgctgggtgatcctccccccgttcctgtgactcgagcgggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagcctctaga

SEQ ID NO: 143 - DNA, 678 bp, Artificial Sequence
  Description of Artificial Sequence Synthetic polynucleotide
actagtatggcgggctccctgtcgtttgtgcggcgcgtgtacctcaccctgtacaactggatcgtgttcgccggctgggcccaggtgctgtactttgccgtcaagacgctcaaggagtccggccacgagaacgtgtacgacgccgtggagaagcccctccagctggcgcaaaccgccgcggtcctggagatcctccacggcctggtcggcctcgtcaggagcccggtctcggccaccctgccgcagatcgggagccgcctctttctgacctggggcattctgtattccttcccggaggtccagagccactttctggtgacctccctcgtgatcagctggtcgatcacggaaatcatccgctacagcttcttcggcctgaaggaggcgctgggcttcgcgcccagctggcacctgtggctccgctattcgagctttctggtgctctaccccaccggcatcacctccgaggtcggcctcatctacctggccctgccgcacatcaagacgtcggagatgtactccgtccgcatgcccaacaccttgaacttttccttcgactttttctacgccacgattctcgtcctcgcgatctacgtccccggttcgccccacatgtaccgctacatgctgggccagcggaagcgggccctgagcaagtccaagcgcgagtgactcgag

SEQ ID NO: 144 - DNA, 969 bp, Artificial Sequence
  Description of Artificial Sequence Synthetic polynucleotide
actagtatggagatctgcacgtacttcaagtcccaacccagctggctgctgctcctgtttttcctgggcagcctccagatcctgaagtcgacgttctccctcctgaagagcctgtacatctacttcctgcgccccggcaagaacctccgccgctacgggtcctgggccattatcaccggcccgaccgacggcatcggcaaggcctttgcgttccagctggcccacaagggcctgaacctggtgctggtggcgcgcaacccggacaagctgaaggacgtctccgacagcatcaggtccaagcatagcaacgtgcagatcaagacggtgatcatggactttagcggcgacgttgacgacggcgtccgccgcatcaaggagaccatcgaggggctggaggtgggcatcctgatcaacaatgccggcatgtcctacccgtacgcgaagtactttcacgaggtcgacgaggagctcgtcaacggcctcatcaaaatcaacgtcgagggcacgaccaaggtgacccaggccgtgctgccgggcatgctggagcgcaagcgcggcgccatcgtcaacatgggcagcggcgcggccgccctgatcccgtcgtaccccttctacagcgtgtatgccggcgcgaagacgtacgtggaccagttcacccggtgcctgcacgtcgagtacaagaagagcggcattgacgtccagtgccaggtcccgctctacgtggccacgaagatgacgaagatccgccgcgcctccttcctggtcgcctcccccgagggctacgccaaggccgccctgcggttcgtggggtacgaggcccggtgcaccccctactggccgcacgccctgatgggctacgtcgtctccgccctgccccagtccgtgttcgagtccttcaacatcaagcgctgcctgcagatccgcaagaagggcatgctgaaggattcgcggaagaaggagtgactcgag

SEQ ID NO: 145 - DNA, 5319 bp, Artificial Sequence
  Description of Artificial Sequence Synthetic polynucleotide
gatttctatcatcaagtttctcatatgtttcacgcgttgctcacaacaccggcaaatgcgttgttgttccctgtttttacaccttgccagagcctggtcaaagcttgacagtttgaccaaattcaggtggcctcatctctctcgcactgatagacattgcagatttggaagacccagtcagtacactacatgcacagccgtttgctcctgcgccatgaacttgccacttttgtgcgccggtcgggggtgatagctcggcagccgccgatcccaaaggtcccgcggcccaggggcacgagaacccccgacacgattaaatagccaaaatcagttagaacggcacctccaccctacccgaatctgacagggtcatcaagcgcgcgaaacaacggcgagggtgcgttcgggaagcgcgcgtagttgacgcaagaagcctgggtcaggctgggagggccgcgagaagatcgcttcctgccgagtctgcacccacgcctcgagcgcaccgtccgcgaacaaccaacccctttgcgcgagccctgacattctttcaattgccaaggatgcacatgtgacacgtatagccattcggctttgtttgtgcctgcttgactcgcgtcatttaattgatttgtgccggtgagccgggagtcggccactcgtctccgagccgcagtcccggcgccagtcccccggcctctgatctgggtccggaagggttggtataggagcggtctcggctatctgaagcccattacccgacactttggccggctgctttccaggcagccgtgtactcttgcgcagtcggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggaattcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagaactcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccactagtatgacggtggccaatcccccggaagccccgttcgacagcgagggttcctcgctggcgcccgacaatgggtccagcaagcccaccaagctgagctccacccggtccttgctgtccatctcctaccgggagctctcgcgttccaagtgcgtgcaggggcgggggcaccttttgttggtgttgtttgggcgggcctcagcactggggtggaggaagaatgcgtgagtgtgcttgcacacctcggcggtttaagatgtaatgcgccaatttcttgctgatgcattcctagacacaaagagtctctcattcgagtctcatcgcggttgtgcgctcctcactccgtgcagccagcagtcgcggtcgttcacttcgcggggggtgccagggaggacggacgtttcggatgagctggagcgccgcatcctcgagtggcagggcgatcgcgccatccacaggtcggttgggtgggaaagggggggcgttggggtcaggtcagaagtcgtgaagttacaggcctgcatttgcacatcctgcgcgcgcctctggccgcttgtcttaagacccttgcactcgcttcctcatgaacccccatgaactccctcctgcaccccacagcgtgctggtggccaacaacggtctggcggcggtcaagttcatccggtcgatccggtcgtggtcgtacaagacgtttgggaacgagcgtgcggtgaagctgatcgcgatggcgacgcccgaggacatgcgcgcggacgcggagcacatccgcatggcggaccagtttgtggaggtccccggcggcaagaacgtgcagaactacgccaacgtgggcctgatcacctcggtggcggtgcgcaccggggtggacgcggtgcctgcagg

SEQ ID NO: 146 - DNA, 811 bp, Artificial Sequence
  Description of Artificial Sequence Synthetic polynucleotide
gattcatatcatcaaatttcgcatatgtttcacgagttgctcacaacatcggcaaatgcgttgttgttccctgtttttacaccttgccagggcctggtcaaagcttgacagtttgaccaaattcaggtggcctcatctctttcgcactgatagacattgcagatttggaagacccagccagtacattacatgcacagccatttgctcctgcaccatgaacttgccacttttgtgcgccggtcgggggtgatagctcggcagccgccgatcccaaaggtcccgcggcccaggggcacgagaccccccgacacgattaaatagccaaaatcagtcagaacggcacctccaccctacccgaatctgacaaggtcatcaaacgcgcgaaacaacggcgagggtgcgttcgggaagcgcgcgtagttgacgcaagaagcctgggtcaggctggagggccgcgagaagatcgcttcctgccgagtctgcacccacgcctcgagcgcaccgtccgcgaacaaccaaccccttttcgcgagccctggcattctttcaattgccaaggatgcacatgtgacacgtatagccattcggctttgtttgtgcctgcttgactcgcgccatttaattgttttgtgccggtgagccgggagtcggccactcgtctccgagccgcagtcccggcgccagtcccccggcctctgatctgggtccggaagggttggtataggagcagtctcggctatctgaagcccgttaccagacactttggccggctgctttccaggcagccgtgtactcttgcgcagtcggtacc

SEQ ID NO: 147 - DNA, 884 bp, Artificial Sequence
  Description of Artificial Sequence Synthetic polynucleotide
actagtatgacggtggccaatcccccggaagccccgttcgacagcgagggttcctcgctggcgcccgacaatgggtccagcaagcccaccaagctgagctccacccggtccctgctgtccatctcctaccgggagctctcgcgttccaagtgcgtacaggggcgagggcaccttttgttggtgttgtttgggcgggcctcggtactgggaggaggaggaatgcgtgcacacctctgcggttttagatgcaatgcgacaagtgcctgctgatgcattttctagacatgaagcatctcgtattcgagtctcaacgcgggtgtgcgctcctcactccgtgcagccagcagtcgcggtcgttcacttcgcggggggtgccagggaggacggacgtttcggatgagctggagcgccgcatcctcgagtggcagggcgatcgcgccatccacaggtcggttgggtgggaaagggggagtaccggggtcaggtcagaagtcgtgcatttacaggcatgcatctgcacatcgtgcgcacgcgcacgtctttggccgcttgtctcaagactcttgcactcgtttcctcatgcaccataatcaattccctcccccctcgcaaactcacagcgtgctggtggccaacaacggtctggcggcggtcaagttcatccggtcgatccggtcgtggtcgtacaagacgtttgggaacgagcgcgcggtgaagctgattgcgatggcgacgcccgagggcatgcgcgcggacgcggagcacatccgcatggcggaccagtttgtggaggtccccggcggcaagaacgtgcagaactacgccaacgtgggcctgatcacctcggtggcggtgcgcaccggggtggacgcggtgcctgcagg

SEQ ID NO: 148 - DNA, 2734 bp, Artificial Sequence
  Description of Artificial Sequence Synthetic polynucleotide
ggtaccgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgccgctagcaccatggccaccgcatccactttctcggcgttcaatgcccgctgcggcgacctgcgtcgctcggcgggctccgggccccggcgcccagcgaggcccctccccgtgcgcgggcgcgccgtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcatgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgataccttattacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcc

SEQ ID NO: 149 - DNA, 9618 bp, Artificial Sequence
  Description of Artificial Sequence Synthetic polynucleotide
catatgtttcacgcgttgctcacaacaccggcaaatgcgttgttgttccctgtttttacaccttgccagagcctggtcaaagcttgacagtttgaccaaattcaggtggcctcatctctctcgcactgatagacattgcagatttggaagacccagtcagtacactacatgcacagccgtttgctcctgcgccatgaacttgccacttttgtgcgccggtcgggggtgatagctcggcagccgccgatcccaaaggtcccgcggcccaggggcacgagaacccccgacacgattaaatagccaaaatcagttagaacggcacctccaccctacccgaatctgacagggtcatcaagcgcgcgaaacaacggcgagggtgcgttcgggaagcgcgcgtagttgacgcaagaagcctgggtcaggctgggagggccgcgagaagatcgcttcctgccgagtctgcacccacgcctcgagcgcaccgtccgcgaacaaccaacccctttgcgcgagccctgacattctttcaattgccaaggatgcacatgtgacacgtatagccattcggctttgtttgtgcctgcttgactcgcgtcatttaattgatttgtgccggtgagccgggagtcggccactcgtctccgagccgcagtcccggcgccagtcccccggcctctgatctgggtccggaagggttggtataggagcggtctcggctatctgaagcccattacccgacactttggccggctgctttccaggcagccgtgtactcttgcgcagtcggtaccgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgccgctagcaccatggccaccgcatccactttctcggcgttcaatgcccgctgcggcgacctgcgtcgctcggcgggctccgggccccggcgcccagcgaggcccctccccgtgcgcgggcgcgccgtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcatgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgataccttattacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggaattccgcctgctcaagcgggcgctcaacatgcagagcgtcagcgagacgggctgtggcgatcgcgagacggacgaggccgcctctgccctgtttgaactgagcgtcagcgctggctaaggggagggagactcatccccaggctcgcgccagggctctgatcccgtctcgggcggtgatcggcgcgcatgactacgacccaacgacgtacgagactgatgtcggtcccgacgaggagcgccgcgaggcactcccgggccaccgaccatgtttacaccgaccgaaagcactcgctcgtatccattccgtgcgcccgcacatgcatcatcttttggtaccgacttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctccttccctgcttctctgtgatcgctgatcagcaacaactagtatgaaggtcacggtggtgagcaggtccggcagggaggtgctcaaggcccccctggacctgccggactccgccacggtcgctgacctccaggaggccttccacaagcgcgcgaagaagttttatcccagccgccagcggctgaccctgccggtggcccccggctccaaggacaagccggtggtgctgaactcgaagaagagcctcaaggagtactgcgacggtaacaccgactcgctcacggtggtgtttaaggacttgggcgcgcaggtctcctaccgcaccctgttcttcttcgagtacctgggccccctgctgatctaccccgtcttctactacttccctgtctataagtacctgggctacggcgaggaccgcgtcatccacccggtgcagacgtatgccatgtactactggtgcttccactactttaagcgcattatggagacgttcttcgtgcaccgcttcagccacgccacctcgcccatcggtaacgtcttccgcaactgcgcctactactggacgttcggcgcctacatcgcttactacgtgaaccaccccctgtacacccccgtgagcgacttgcagatgaagatcggcttcgggttcggcctcgtgtttcaggtggcgaacttctactgccacatcctgctgaagaatctgcgcgacccgaacggcagcggcggttaccagatcccgcgcggcttcctgttcaacatcgtcacgtgcgcgaactacaccacggagatctaccagtggctcggctttaacatcgccacgcagaccatcgccggctacgtgttcctcgcggtggccgccctgattatgaccaactgggccctcggcaagcactcgcggctccggaagatcttcgacggcaaggacggcaagccgaagtacccccgccgctgggtgatcctccccccgttcctgtgactcgagcgggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggatctagatacgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcatcaaaccgagttgtgcacaagggcgactaattttttagtcgggaaacaacccgcttccagaagcatccggacgggggtagcgaggctgtgtcgagcgccgtggggatctggccggtgaggtgcccgaaatccgtgtacagctcagcggctgggatcatcgacccccgggatcatcgaccccgtgggccgggcccccggaccctataactaaaagccgacgccagtgcaaaaccacaaacatttactccttaatcctccctcctccttcatacacacccacaagtaatcaactcaccactagtatggagatctgcacgtacttcaagtcccaacccagctggctgctgctcctgtttttcctgggcagcctccagatcctgaagtcgacgttctccctcctgaagagcctgtacatctacttcctgcgccccggcaagaacctccgccgctacgggtcctgggccattatcaccggcccgaccgacggcatcggcaaggcctttgcgttccagctggcccacaagggcctgaacctggtgctggtggcgcgcaacccggacaagctgaaggacgtctccgacagcatcaggtccaagcatagcaacgtgcagatcaagacggtgatcatggactttagcggcgacgttgacgacggcgtccgccgcatcaaggagaccatcgaggggctggaggtgggcatcctgatcaacaatgccggcatgtcctacccgtacgcgaagtactttcacgaggtcgacgaggagctcgtcaacggcctcatcaaaatcaacgtcgagggcacgaccaaggtgacccaggccgtgctgccgggcatgctggagcgcaagcgcggcgccatcgtcaacatgggcagcggcgcggccgccctgatcccgtcgtaccccttctacagcgtgtatgccggcgcgaagacgtacgtggaccagttcacccggtgcctgcacgtcgagtacaagaagagcggcattgacgtccagtgccaggtcccgctctacgtggccacgaagatgacgaagatccgccgcgcctccttcctggtcgcctcccccgagggctacgccaaggccgccctgcggttcgtggggtacgaggcccggtgcaccccctactggccgcacgccctgatgggctacgtcgtctccgccctgccccagtccgtgttcgagtccttcaacatcaagcgctgcctgcagatccgcaagaagggcatgctgaaggattcgcggaagaaggagtgactcgagcgggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagatatcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagaactcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccactagtatgacggtggccaatcccccggaagccccgttcgacagcgagggttcctcgctggcgcccgacaatgggtccagcaagcccaccaagctgagctccacccggtccttgctgtccatctcctaccgggagctctcgcgttccaagtgcgtgcaggggcgggggcaccttttgttggtgttgtttgggcgggcctcagcactggggtggaggaagaatgcgtgagtgtgcttgcacacctcggcggtttaagatgtaatgcgccaatttcttgctgatgcattcctagacacaaagagtctctcattcgagtctcatcgcggttgtgcgctcctcactccgtgcagccagcagtcgcggtcgttcacttcgcggggggtgccagggaggacggacgtttcggatgagctggagcgccgcatcctcgagtggcagggcgatcgcgccatccacaggtcggttgggtgggaaagggggggcgttggggtcaggtcagaagtcgtgaagttacaggcctgcatttgcacatcctgcgcgcgcctctggccgcttgtcttaagacccttgcactcgcttcctcatgaacccccatgaactccctcctgcaccccacagcgtgctggtggccaacaacggtctggcggcggtcaagttcatccggtcgatccggtcgtggtcgtacaagacgtttgggaacgagcgtgcggtgaagctgatcgcgatggcgacgcccgaggacatgcgcgcggacgcggagcacatccgcatggcggaccagtttgtggaggtccccggcggcaagaacgtgcagaactacgccaacgtgggcctgatcacctcggtggcggtgcgcaccggggtggacgcggtgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaat

SEQ ID NO: 150 - DNA, 678 bp, Artificial Sequence; identical to SEQ ID NO: 143
  Description of Artificial Sequence Synthetic polynucleotide

SEQ ID NO: 151 - DNA, 811 bp, Artificial Sequence; identical to SEQ ID NO: 146
  Description of Artificial Sequence Synthetic polynucleotide

SEQ ID NO: 152 - DNA, 884 bp, Artificial Sequence; identical to SEQ ID NO: 147
  Description of Artificial Sequence Synthetic polynucleotide

SEQ ID NO: 153 - protein, 310 aa, Alliaria petiolata
MKVTVVSRSGREVLKAPLDLPDSATVADLQEAFHKRAKKFYPSRQRLTLPVAPGSKEKPVVLNSKKSLKEYCDGNTDSLTVVFKDLGTQVSYRTLFFFEYLGPLLIYPVFYYFPVYKFLGYGEDRVIHPVQTYAMYYWCFHYFKRILETFFVHRFSHATSPIANVFRNCAYYWTFGAYIAYYVNHPLYTPVSDLQMKIGFGFGLVCQVANFYCHILLKNLRDPNGSGGYQIPRGFLFNIVTCANYTTEIYQWLGFNIATQTVAGYVFLTVAALIMTNWALGKHSRLRKIFDGKDGKPKYPRRWVILPPFL

SEQ ID NO: 154 - protein, 310 aa, Arabidopsis thaliana
MKVTVVSRSGREVLKAPLDLPDSATVADLQEAFHKRAKKFYPSRQRLTLPVTPGSKDKPVVLNSKKSLKEYCDGNNNSLTVVFKDLGAQVSYRTLFFFEYLGPLLIYPVFYYFPVYKFLGYGEDCVIHPVQTYAMYYWCFHYFKRILETFFVHRFSHATSPIGNVFRNCAYYWSFGAYIAYYVNHPLYTPVSDLQMKIGFGFGLVCQVANFYCHILLKNLRDPSGAGGYQIPRGFLFNIVTCANYTTEIYQWLGFNIATQTIAGYVFLAVAALIMTNWALGKHSRLRKIFDGKDGKPKYPRRWVILPPFL

SEQ ID NO: 155 - protein, 310 aa, Crambe abyssinica
MKVTVVSRSGREVLKAPLDLPDSATVADLQEAFHKRAKKFYPSRQRLTLPVAPGSKDKPVVLNSKKSLKEYCDGNTDSLTVVFKDLGAQVSYRTLFFFEYLGPLLIYPVFYYFPVYKYLGYGEDRVIHPVQTYAMYYWCFHYFKRIMETFFVHRFSHATSPIGNVFRNCAYYWTFGAYIAYYVNHPLYTPVSDLQMKIGFGFGLVFQVANFYCHILLKNLRDPNGSGGYQIPRGFLFNIVTCANYTTEIYQWLGFNIATQTIAGYVFLAVAALIMTNWALGKHSRLRKIFDGKDGKPKYPRRWVILPPFL

SEQ ID NO: 156 - protein, 310 aa, Crambe cordifolia
MKVTVVSRSGREVLKAPLDLPDSATVADLQEAFHKRAKKFYPSRQRLTLPVAPGSKDKPVVLNSKKSLKEYCDGNTDSLTVVFKDLGAQVSYRTLFFFEYLGPLLIYPVFYYFPVYKYLGYGEDRVIHPVQTYAMYYWCFHYFKRIMETFFVHRFSHATSPIGNVFRNCAYYWTFGAYIAYYVNHPLYTPVSDLQMKIGFGFGLVCQVANFYCHILLKNLRDPNGSGGYQIPRGFLFNIVTCANYTTEIYQWLGFNIATQTIAGYVFLAVAALIMTNWALGKHSRLRKIFDGKDGKPKYPRRWVILPPFL

SEQ ID NO: 157 - protein, 310 aa, Erysimum allioni
MKVTVVSRSGREVLKAPLDLPDSATVADLQEAFHKRAKKFYPSRQRLTLPVAPGSKDKPVVLNSKKSLKEYCDGNTDSLTVVFKDLGAQVSYRTLFFFEYLGPLLIYPVFYYLPVYKFLGYGEDRVIHPVQTYAMYYWCFHYFKRILETFFVHRFSHATSPIGNVFRNCAYYWSFGAFIAYYVNHPLYTPVSDLQMKIGFGFGLVCQVANFYCHILLKNLRDPNGSGGYQIPRGFLFNIVTCANYTTEIYQWLGFNIATQTIAGYVFLAVAALIMTNWALGKHSRLRKIFDGKDGKPKYPRRWVILPPFL

SEQ ID NO: 158 - protein, 305 aa, Prototheca moriformis
MPLEILIKTRDGRPAFSRDGGAITVDSTSATVQEVKGLIARAKKLSPARLRLTLPAPAGTRPTVLEDKKTLGDYGLHDGASLVLKDLGPQIGYQMVFFWEYFGPLAIYPLFYFLPSLIYGRSTEHVFAQKAALAFWTFHYAKRILETFFVHKFGHATMPVRNLVKNCSYYWSFGAFISYFVNHPLYAAPPAAQTAVAFVAATLCTLSNFKCHLILSNLRAPGGSGYVIPRGFLFDYVTCANYTAEIWSWIFFSIGTQCLPALIFTVAGAAQMAIWAGGKHRRLKKLFDGKEGRERYPKRYIMLPP

SEQ ID NO: 159 - protein, 305 aa, Prototheca moriformis
MPLEILVKTRNGRPAFSRDGGAITVDSTSATVQEVKGLIARAKKLSPARLRLTLPAPAGTRPTVLEDKKTLGDYGLHDGASLVLKDLGPQIGYQMVFFWEYFGPLAIYPLFYFLPSLIYGRPTEHVFAQKAALAFWTFHYGKRILESFFVHKFGHATMPVRNLVKNCSYYWSFGAFISYFVNHPLYSAPPAAQTAVAFVAATLCTLSNFKCHLILSNLRAPGGSGYVIPRGFLFDYVTCANYTAEIWSWIFFSIGTQCLPALVFTVAGAAQMAIWAGGKHCRLKKLFDGKEGRERYPKRYIMFPP

SEQ ID NO: 160 - protein, 221 aa, Alliaria petiolata
MAGFLSVVRRVYLTLYNWIVFAGWAQVLFLAIKTLKETGHENVYDAIEKPLQLAQTAAVLEILHGLVGLVRSPVSATLPQIGSRLFLTWGILYSFPEVRSHFLVTSLVISWSITEIIRYSFFGFKEALGFAPAWHLWLRYSSFLLLYPTGITSEVGLIYLALPHIKTSEMYSVRMPNTLNFSFDYFYATILVLAIYVPGSPHMYRYMLGQRKRALSKSKRE

SEQ ID NO: 161 - protein, 221 aa, Arabidopsis thaliana
MAGFLSVVRRVYLTLYNWIVFAGWAQVLYLAITTLKETGYENVYDAIEKPLQLAQTAAVLEILHGLVGLVRSPVSATLPQIGSRLFLTWGILYSFPEVRSHFLVTSLVISWSITEIIRYSFFGFKEALGFAPSWHLWLRYSSFLLLYPTGITSEVGLIYLALPHIKTSEMYSVRMPNILNFSFDFFYATILVLAIYVPGSPHMYRYMLGQRKRALSKSKRE

SEQ ID NO: 162 - protein, 221 aa, Crambe abyssinica
MAGSLSFVRRVYLTLYNWIVFAGWAQVLYFAVKTLKESGHENVYDAVEKPLQLAQTAAVLEILHGLVGLVRSPVSATLPQIGSRLFLTWGILYSFPEVQSHFLVTSLVISWSITEIIRYSFFGLKEALGFAPSWHLWLRYSSFLVLYPTGITSEVGLIYLALPHIKTSEMYSVRMPNTLNFSFDFFYATILVLAIYVPGSPHMYRYMLGQRKRALSKSKRE

SEQ ID NO: 163 - protein, 221 aa, Crambe cordifolia
MAGSFSFIRRVYLTLYNWIVFAGWAQVLYFAVKTLKETGHENVYDAVEKPLQLAQTAAVLEILHGLVGLVRSPVSATLPQIGSRLFLTWGILYSFPEVQSHFLVTSLVISWSITEIIRYSFFGLKEALGFAPSWHLWLRYSSFLVLYPTGITSEVGLIYLALPHIKTSEMYSVRMPNTLNFSFDFFYATILVLAIYVPGSPHMYRYMLGQRKRALSKSKRE

SEQ ID NO: 164 - protein, 221 aa, Erysimum allioni
MAGFLSVVRRVYLTLYNWIVFAGWAQVLFLAIKTLKETGHENVFDAIEKPLQLAQTAAVLEILHGLVGLVRSPVSATLPQIGSRLFLTWGILYSFPEVRSHFLVTSLVISWSITEIIRYSFFGFKEALGFAPAWHLWLRYSSFLLLYPTGITSEVGLIYLALPHMKTSEMYSVRMPNTLNFSFDYFYATILVLAIYVPGSPHMYRYMLGQRKRALSKSKRE

SEQ ID NO: 165 - protein, 221 aa, Erysimum sp.
  Erysimum golden gem
MAGFLSVVRRVYLTLYNWIVFAGWAQVLFLAIKTLKETGHENVYDAIEKPLQLAQTAAVLEILHGLVGLVRSPVSATLPQIGSRLFLTWGILYSFPEVRSHFLVTSLVISWSITEIIRYSFFGFKEALGFAPAWHLWLRYSSFLLLYPTGITSEVGLIYLALPHMKTSEMYNVRMPNTLNFSFDYFYATILVLAIYVPGSPHMYRYMLGQRKRALSKSKRE

SEQ ID NO: 166 - protein, 221 aa, Erysimum sp.; identical to SEQ ID NO: 160
  Erysimum helveticum

SEQ ID NO: 167 - protein, 235 aa, Prototheca moriformis
MSLRSAYLTIYNGSLALGWAYLLWLSVSVLAAGGSLWDLWKTVEVPLKIIQTAAIAEVVHASVGIVRSPPLVTALQVASRVFLVWGVVNLAPEVATGSQVAAVPIPGVGRVGLSFATLVIAWALSEIIRYGHFAAKEAGIASKLLLWLRYTGFLVLYPLGVSSELTMVYLVAPYVKKHGILSLEMPNAANFAFSYYAALWIVSLTYIPGFPMLYGYMLKQRKKMLGGGAKAKKLA

SEQ ID NO: 168 - protein, 318 aa, Alliaria petiolata
MEICTYFKSQPTWLLILFLLGSISISKFTFTLLRSFYIYFLRPAKNLRKYGSWALITGPTDGIGKAFAFQLAQKGFNLILVGRNPEKLKDVSESIRSKYNKTQILTVVMDFSGDIDEGVKRIKETIEGLEVGVLINSAGISYPYAKYFHEVDQELLNNLIKINVEGTTKVTQAVLPNMLARKRGAIVNMGSGAAALIPSYPFYSVYAGAKTYVDQFTKCLHVEYKKSGIDVQCQVPLYVATKMTKIRKASFLVASPEGYAKAALRFVGYEAQCTPYWPHALMGAVISALPESIFESFNIKRCLQIRKKGLQKDSMKKE

SEQ ID NO: 169 - protein, 318 aa, Arabidopsis thaliana
MEICTYFKSQPTWLLILFVLGSISIFKFIFTLLRSFYIYFLRPSKNLRRYGSWAIITGPTDGIGKAFAFQLAQKGLNLILVARNPDKLKDVSDSIRSKYSQTQILTVVMDFSGDIDEGVKRIKESIEGLDVGILINNAGMSYPYAKYFHEVDEELINNLIKINVEGTTKVTQAVLPNMLKRKKGAIINMGSGAAALIPSYPFYSVYAGAKTYVDQFTKCLHVEYKKSGIDVQCQVPLYVATKMTKIRRASFLVASPEGYAKAALRFVGYEAQCTPYWPHALMGAVVSALPESVFESFNIKRCLQIRKKGLQKDSMKKE

SEQ ID NO: 170 - protein, 319 aa, Brassica napus
MEICTYFKSQPTWLLVLFSLGSISILRFTFTLLTSLYIYFLRPGKNLRRYGSWAIITGPTDGIGKAFAFQLAQKGLNLVLVARNPDKLKDVSDSIQAKYSNTQIKTVVMDFSGDIDGGVRRIKEAIEGLEVGILINNAGVSYPYAKYFHEVDEEMLGNLIKINVEGTTKVTQAVLVNMLKRKRGAIVNMGSGAAALIPSYPFYSVYAGAKTYVDQFSRCLHVEYKKSGIDVQCQVPLYVATKMTKIRRASFLVASPEGYAKAALRFVGYEPRCTPYWPHALMGYVVSALPESVFESFNIKRCLQIRKKGMLKDSISKKE

SEQ ID NO: 171 - protein, 319 aa, Brassica napus
MEICTYFKSQPTWLLVLFSLGSISILRFTLTLLTSLYIYFLRPGKNLRRYGSWAIITGPTDGIGKAFAFQLAQKGLHLVLVARNPDKLKAVSDSIQAKHSTTQIKTVLMDFSGDIDAGVRRIKEAIEGLEVGILINNAGVSYPYAKYFHEVDEELLGNLIKINVEGTTKVTQAVLVNMLKRKRGAIVNMGSGAAALIPSYPFYSVYAGAKTYVDQFSRCLHVEYKKSGIDVQCQVPLYVATKMTKIRRASFLVASPEGYAKAALRFVGYEPRCTPYWPHALMGYVVSALPESVFESFNIKRCLQIRKKGMLKDSSSKKE

SEQ ID NO: 172 - protein, 318 aa, Crambe abyssinica
MEICTYFKSQPSWLLLLFFLGSLQILKSTFSLLKSLYIYFLRPGKNLRRYGSWAIITGPTDGIGKAFAFQLAHKGLNLVLVARNPDKLKDVSDSIRSKHSNVQIKTVIMDFSGDVDDGVRRIKETIEGLEVGILINNAGMSYPYAKYFHEVDEELVNGLIKINVEGTTKVTQAVLPGMLERKRGAIVNMGSGAAALIPSYPFYSVYAGAKTYVDQFTRCLHVEYKKSGIDVQCQVPLYVATKMTKIRRASFLVASPEGYAKAALRFVGYEARCTPYWPHALMGYVVSALPQSVFESFNIKRCLQIRKKGMLKDSRKKE

SEQ ID NO: 173 - protein, 318 aa, Crambe cordifolia
MEICTYFKSQPSWLLVLFALGSLQILKFTFSLLTSLYIYFLRPGKNLRRYGSWAIITGPTDGIGKAFAFQLAHKGLNLVLVARNPDKLRDVSDSIRSKYSNVEIKTVLMDFSGDVDDGVRRIKETIEGLEVGILINNAGMSYPYAKYFHEVDEELVNGLIKINVEGTTKVTQAVLPGMLKRKRGAIVNMGSGAAALIPSYPFYSVYAGAKTYVDQFTRCLHVEYKKSGIDVQCQVPLYVATKMTSIRRASFLVASPEGYAKAALRFVGYEARCTPYWPHALMGYVVSALPESVFESFNIKRCLQIRKKGMLKDSRKKE

SEQ ID NO: 174 - protein, 318 aa, Erysimum allioni
MEICTYFKSQPTWLLFLFALGSISIFKFIFSLLRSFYIYFLRPAKNLRRYGSWAIVTGPTDGIGKAFAFQLAQKGLNLVLVARNPDKLNDVSDSIRSKYSNIQIKTVIMDFSGDIDEGVKRIKETIEGLEIGILINNAGMSYPYAKYFHEVDEEMLTNLIKINVEGTTKVTQAVLPIMLQRKRGAIVNMGSGAAALIPSYPFYSVYAGAKTYVDQFTRCLHVEYKKSGIDVQCQVPLYVATKMTSIRRASFLVASPEGYAKAALRFVGYEARCTPYWPHALMGFVVSALPESVFESFNIKRCLQIRKKGLLKDSRKKE

SEQ ID NO: 175 - protein, 339 aa, Prototheca moriformis
MDAQGYLVKASESPAWTYLVLLASALFAIKVVGFVLTVLGGLYAHFLRKGKKLRRYGDWAVVTGATDGIGKAYAEALAKQKLRLVLISRTESRLEEEARLLQDKFGVEVKIIPADLSSSDEAVFARIGKGLEGLDIGILVNNAGMSYPHPEYLHLVDDETLTNLINLNVATLTKLCKMVLGGMKERGRGLVVNVGSGVASAIPSGPLLSAYTASKAYVDQLSESLNDEYKEFGVQVQNQAPLFVATKMSKIRKPRIDAPTPGTWAAAAVRAMGFETLSFPYWFHALQAAVVERLPEAMIRYQVMQIHRSLRRAAYKKKARASAAALADEGVSAEPKKDL

SEQ ID NO: 176 - protein, 326 aa, Zea mays
MAGTCAHVEFLRAQPAWALALAAVGLLVAVRAAARFALWVYAAFLRPGKPLRRRYGAWAVVTGATDGIGRAVAFRLAASGLGLVLVGRNQEKLAAVAAEIKARHPKVPEVRTFVLDFAGEGLAAAVEALKDSIRGLDVGVLVNNAGVSYPYARYFHEVDEELMRTLIRVNVEGVTRVTHAVLPAMVERKRGAIVNIGSGAASVVPSDPLYSVYAATKAYVDQFSRCLYVEYKSKGIDVQCQVPLYVATKMASIRKSSFMVPSADTYARAAVRHIGYEPRCTPYWPHSVVWFLISILPESLIDSVRLGMCIKIRKKGLAKDAKKKAL

SEQ ID NO: 177 - protein, 1145 aa, Prototheca moriformis
MTVANPRKPPLAFQPAVAVVHFAGVPGRTDVSDELERRILEWQGDRAIHSVLVANNGLAAVKFIRSIRSWSYKTFGNERAVKLIAMATPEDMRADAEHIRMADQFVEVPGGKNVQNYANVGLITSVAVRTGVDAVWPGWGHASEFPELPESLGATPSQIRFVGPPAGPMAALGDKVGSTILAQAAGVPTLAWSGSGVSIAYADCPRGEIPPEVYRRACIDSLEAALACCERIGYPVMLKASWGGGGKGIRKVLSADEVKLAYTQVCGEVPGSPVFAMKLAPQSRHLEVQLLCDAHGQVCSLYSRDCSVQRRHQKVVEEGPVTAAPPEVLEGMERCARSLARAVGYVGAATVEYLYMVETREYCFLELNPRLQVEHPVTEMITGVNIPAAQLLVAAGVPLHRIPDIRRLYRAPLAEDGPIDFEDDAARLPPNGHVLAVRVTAENAHDGFKPTSGAIEEISFRSTPDVWGYFSVKGGSAVHEFADSQFGHLFARGDSREAAVRAMVVALKEIKIRGEIQTPVDYVARMIQTDDFLGNRHHTGWLDARIAAQVGAERPPWFLVVIAGAVLRATRAVNERAAAFLDHLRKGQLPPSEVPLTRVAEEFVVDGTKYAVDVTRTGPQAYRVALRDPEARAPGHARRESLASAAGAASSVDVIARTLQDGGLLLQVDGRVHVLHSEEEALGTRLVIDSATCLLSNEHDPSQLVAVSTGKLVRHLVAEGEHVRADEPYAEVEVMKMMMTLLAPAAGAVRWEVAEGAALAPGLLLARLELDDPAAAAQDVLDGFEGDVPAVVAALLAALDDPALSLVLLDEALAVASTPRFVVKRRLTPAARCTARFEEAARAALASAASEEARAALDPLLTPLLELTAAHRYLDVEERFESGGAKTDQEVIDGLRGTHSADPGKVLEIVVAHQSAGPRADLVHRLLNALRARRLLERSLLGELRVLVARALSGLDMFSDATLRGLCLGESPGEPVSPRAELASALAAESSVASPTSRRSTLAEGLYVGLGNLAAAAASSVEARMAMLVEAPAAVDDALATLLDHPDPVLQRRALSTYVRRIYFPGVLHEPQVVGLGRRGSVAAVWAHAGARPARPRATAARSSMAGLAPGTLHVVLTAEGAAALQLDAAAQAALGTLDVSGYVA

SEQ ID NO: 178 - protein, 1182 aa, Prototheca moriformis
MTVANPPEAPFDSEALAFQPAVAVVHFAGVPGRTDVSDELERRILEWQGDRAIHSVLVANNGLAAVKFIRSIRSWSYKTFGNERAVKLIAMATPEDMRADAEHIRMADQFVEVPGGKNVQNYANVGLITSVAVRTGVDAVWPGWGHASEFPELPESLGATPSQIRFVGPPAGPMAALGDKVGSTILAQAAGVPTLAWSGSGVSIAYADCPRGEIPPEVYRRACIDSLEAALACCERIGYPVMLKASWGGGGKGIRKVLSADEVKLAYTQVCGEVPGSPVFAMKLAPQSRHLEVQLLCDAHGQVCSLYSRDCSVQRRHQKVVEEGPVTAAPPEVLEGMERCARSLARAVGYVGAATVEYLYMVETQEYCFLELNPRLQVEHPVTEMITGVNIPAAQLLVAAGVPLHRIPDIRRLYRAPLAEDGPIDFEDDAARLPPNGHVLAVRVTAENAHDGFKPTSGAIEEISFRSTPDVWGYFSVKGGSAVHEFADSQFGHLFARGDSREAAVRAMVVALKEIKIRGEIQTPVDYVARMIQTDDFLGNRHHTGWLDARIAAQVGAERPPWFLVVIAGAVLRATRAVNERAAAFLDHLRKGQLPPSEVPLTRVAEEFVVDGTKYAVDVTRTGPQAYRSLASAAGAASSVDVIARTLQDGGLLLQVDGRVHVLHSEEEALGTRLVIDSATCLLSNEHDPSQLVAVSTGKLVRHLVAEGEHVRADEPYAEVEVMKMMMTLLAPAAGAVRWEVAEGAALAPGLLLARLELDDPAAVRRAEPFRGAFPDLGPPSPDFDGLAARYRSALEAAQDVLDGFEGDVPAVVAALLAALDDPALSLVLLDEALAVASTPRFVVKRRLTPAARCTARFEEAARAALASAASEEARAALDPLLTPLLELTAAHVGGPEGHARRVARQLLERYLDVEERFESGGAKTDQEVIDGLRGTHSADPGKVLEIVVAHQSAGPRADLVHRLLNALRARRLLERSLLGELRVLVARALSGLDMFSDATLRGLCLGESPGEPVSPRAELASALAAESSVASPTSRRSTLAEGLYVGLGNLAAAAASSVEARMAMLRRALSTYVRRIYFPGVLHEPQVVGLGRRGSVAAVWAHAGARPGAPARHGGALVVPALRALPDALAELAHLRAQTSMAGLAPGTLHVALTAEGAAALQLDAAAQAALGTLDVSGYVA

SEQ ID NO: 179 - protein, 1105 aa, Prototheca moriformis
PEHQQQPGLPDATVVAASAAAAVSALAPALREAGYNAVSFLTKRGGVEPLRVVFYDGTSSGSAAASDASSTQTATGWSLDPVLGTVEPPTAEALELACLAGRAGASHDASRNRQWHMWTVAERAGKRSAVLRRTFLRGAVRSLGRPALLSAAYAGNGPAVAAAALAELEQTVEAAAAELERLGKGRVGGASPDWTHAFFSVLSSLPLGASEPREEGAVARALAAGAAAIASRHVAALRRAALAQWEVCLRTGSGGASHREGGWRVVVSSPTGHEAGEAFVDVYREAADGTLRAVNPSLRAPGPLDGQSVLAPYPTLAPLQQRRLTARRHKTTYAYDFPAVFEDALRSIWLQRAVELGATGLEAAREDLLPPGNRLVTAEELVLDTEEAIYENGAAHIRLTDRAPSMNDLGVVAWRLTLATPECPRGRAVVAIANDITYNSGSFGPREDAFFKAATEYALAERLPVVYLAANSGARVGLAEEVKRCLRVEWSVPGDPTKGHKYLYLDDEDYRSITSRAAGRTLPVSCSAKVGADGRTRHVLHDVIGLEDGLGVECLSGSGAIAGAFARAFREGFTVTLVSGRTVGIGAYLARLGRRCVQRREQPIILTGFAALNKLLGREVYTSQQQLGGPRVMGANGVSHHVVDDDLQGVHTVLRWLAYTPARVGELPPTLRAADPVDRRVTYAPVENEKLDPRLAVAGGDAPEPVTGLFDRGSWTEAQAGWAQTVVTGRARLGGIPVGVVAVEVNAVSLHIPADPGMPDSAERTIPQAGQVWFPDSALKTAQAIEEFGLEGLPLFILANWRGFSGGQRDLFEGVLQAGSQIVEMLRTYRRPVTVYLGPGCELRGGAWVVLDSQINPASIEMYADPTAQGAVLEPQGVVEIKFRTPDLLAAMHRLDEKIIALKSDDSPSALAAIKAREAELLPVYSQVAHQFAQMHDGPVRMLAKGVLRGIVPWSAARAFLATRLRRRLAEEALLRQIAAADASVEHADALAMLRSWFLSSPPTGGAPGAPGALGALLKETVVAPPDAGEAPLALWQDDLAFLDWSEAEAGASRVALELKSLRVNVAMRSVDRLCQTPEGTAGLVKGLDEAIKSNPSLLLCLRSL

SEQ ID NO: 180 - protein, 1067 aa, Prototheca moriformis
PEHQQQPGCRTPPSAVSFLTKRGGVEPLRVVFYDGTSSGSAAASDSSSTQTGPSTGWSLDPVLGTVEPPTAEALELACLAGRAGASHDASRNRQWHMWTVAERAGKRSAVLRRTFLRGAVRSLGRPALLSAAYAGNGPAVAAAALAELEQTVEAAAAELERLGKGRVGGASPDWTHAFFSVLSSLPLGASEPREEGAVARALAAGAAAIASRHVAALRRAALAQWEVCLRTGSGGASHREGGWRVVVSSPTGHEAGEAFVDVYREAADGTLRAVNPSLRAPGPLDGQSVLAPYPTLAPLQQRRLTARRHKTTYAYDFPAVFEDALRSIWLQRAVELGATGLEAAREDLLPPGHRLVTAEELVLDTEEAVYENGAAHIRLTDRAPSMNDLGVVAWRLTLATPECPRGRAVVAIANDITYNSGSFGPREDAFFKAATEYALAERLPVVYLAANSGARVGLAEEVKRCLRVEWSVPGDPTKGHKYLYLDDEDYRSITSRAAGRTLPVSCSAKVGADGRTRHVLHDVIGLEDGLGVECLSGSGAIAGAFARAFREGFTVTLVSGRTVGIGAYLARLGRRCVQRREQPIILTGFAALNKLLGREVYTSQQQLGGPRVMGANGVSHHVVDDDLQGVHTVLRWLAYTPARVGELPPTLRAADPVDRRVTYAPVENEKLDPRLAVAGGDAPEPVTGLFDRGSWTEAQAGWAQTVVNAVSLHIPADPGMPDSAERTIPQAGQVWFPDSALKTAQAIEEFGLEGLPLFILANWRGFSGGQRDLFEGVLQAGSQIVEMLRTYRRPVTVYLGPGCELRGGAWVVLDSQINPASIEMYADPTAQGAVLEPQGVVEIKFRTPDLLAAMHRLDEKIIALKSDDSPSALAAIKARESELLPVYSQVAHQFAQMHDGPVRMLAKGVLRGIVPWSAARAFLATRLRRRLAEEALLRQIAAADASVEHADALAMLRSWFLSSPPTGGAPGAPGALGALLKETVVAPPDAGEAPLALWQDDLAFLDWSEAEAGASRVALELKSLRVNVAMRSVDRLCQTPEGTAGLVKGLDEAIKSNPSLLLCLRSL

(19) United States
(12) Patent Application Publication
(10) Pub. No.: US 2016/0348119 A1
(43) Pub. Date: Dec. 1, 2016

(54) Oleaginous Microalgae Having an LPAAT Ablation

(71) Applicant: TerraVia Holdings, Inc., South San Francisco, CA (US)

(72) Inventors: Scott Franklin, Woodside, CA (US); Riyaz Bhat, South San Francisco, CA (US); Xinhua Zhao, Dublin, CA (US)

(21) Appl. No.: 15/092,538

(22) Filed: Apr. 6, 2016

Related U.S. Application Data
(60) Provisional application No. 62/143,711, filed on Apr. 6, 2015.
(60) Provisional application No. 62/145,723, filed on Apr. 10, 2015.

(51) Int. Cl. C12N 15/52; C11C 1/00

(52) U.S. Cl. CPC C12N 15/52; C11C 1/002

(57) ABSTRACT

Recombinant DNA techniques are used to produce oleaginous recombinant cells that produce triglyceride oils having desired fatty acid profiles and regiospecific or stereospecific profiles. Genes manipulated include those encoding stearoyl-ACP desaturase, delta 12 fatty acid desaturase, acyl-ACP thioesterase, ketoacyl-ACP synthase, lysophosphatidic acid acyltransferase, ketoacyl-CoA reductase, hydroxyacyl-CoA dehydratase, and/or enoyl-CoA reductase. The oil produced can have enhanced oxidative or thermal stability, or can be useful as a frying oil, shortening, roll-in shortening, tempering fat, cocoa butter replacement, as a lubricant, or as a feedstock for various chemical processes. The fatty acid profile can be enriched in midchain profiles or the oil can be enriched in triglycerides of the saturated-unsaturated-saturated type.

### CROSS-REFERENCE TO RELATED APPLICATIONS

This application claims the benefit under 35 USC 119(e) of U.S. Provisional Patent Application No. 62/143,711, filed Apr. 6, 2015, and U.S. Provisional Patent Application No. 62/145,723, filed Apr. 10, 2015, each of which is incorporated herein by reference in its entirety.

### REFERENCE TO A SEQUENCE LISTING

This application includes a list of sequences, as shown at the end of the detailed description.

### FIELD OF THE INVENTION

Embodiments of the present invention relate to oils/fats, fuels, foods, and oleochemicals and their production from cultures of genetically engineered cells. Specific embodiments relate to oils with a high content of triglycerides bearing fatty acyl groups upon the glycerol backbone in particular regiospecific patterns, highly stable oils, oils with high levels of oleic or mid-chain fatty acids, and products produced from such oils.

### BACKGROUND OF THE INVENTION

PCT Publications WO2008/151149, WO2010/06031, WO2010/06032, WO2011/150410, WO2011/150411, WO2012/061647, WO2012/061647, WO2012/106560, and WO2013/158938 disclose oils and methods for producing those oils in microbes, including microalgae. These publications also describe the use of such oils to make foods, oleochemicals and fuels.

Certain enzymes of the fatty acyl-CoA elongation pathway function to extend the length of fatty acyl-CoA molecules. Elongase-complex enzymes extend fatty acyl-CoA molecules in 2 carbon additions, for example myristoyl-CoA to palmitoyl-CoA, stearoyl-CoA to arachidyl-CoA, or oleoyl-CoA to eicosanoyl-CoA, eicosanoyl-CoA to erucyl-CoA. In addition, elongase enzymes also extend acyl chain length in 2 carbon increments. KCS enzymes condense acyl-CoA molecules with two carbons from malonyl-CoA to form beta-ketoacyl-CoA. KCS and elongases may show specificity for condensing acyl substrates of particular carbon length, modification (such as hydroxylation), or degree of saturation. For example, the jojoba (Simmondsia chinensis) beta-ketoacyl-CoA synthase has been demonstrated to prefer monounsaturated and saturated C18- and C20-CoA substrates to elevate production of erucic acid in transgenic plants (Lassner et al., Plant Cell, 1996, Vol 8(2), pp. 281-292), whereas specific elongase enzymes of Trypanosoma brucei show preference for elongating short and midchain saturated CoA substrates (Lee et al., Cell, 2006, Vol 126(4), pp. 691-9).

The type II fatty acid biosynthetic pathway employs a series of reactions catalyzed by soluble proteins with intermediates shuttled between enzymes as thioesters of acyl carrier protein (ACP). By contrast, the type I fatty acid biosynthetic pathway uses a single, large multifunctional polypeptide.

The oleaginous, non-photosynthetic alga, Prototheca moriformis, stores copious amounts of triacylglyceride oil under conditions when the nutritional carbon supply is in excess, but cell division is inhibited due to limitation of other essential nutrients. Bulk biosynthesis of fatty acids with carbon chain lengths up to C18 occurs in the plastids; fatty acids are then exported to the endoplasmic reticulum where (if it occurs) elongation past C18 and incorporation into triacylglycerides (TAGs) is believed to occur. Lipids are stored in large cytoplasmic organelles called lipid bodies until environmental conditions change to favor growth, whereupon they are mobilized to provide energy and carbon molecules for anabolic metabolism.

### SUMMARY OF THE INVENTION

In accordance with an embodiment, there is a cell, optionally a microalgal cell, which produces at least 20% oil by dry weight. The oil has a fatty acid profile with 5% or less of saturated fatty acids, optionally less than 4%, less than 3.5%, or less than 3% of saturated fatty acids. The fatty acid profile can have (a) less than 2.0% C16:0; (b) less than 2% C18:0; and/or (c) a C18:1/C18:0 ratio of greater than 20. Alternately, the fatty acid profile can have (a) less than 1.9% C16:0; (b) less than 1% C18:0; and/or (c) a C18:1/C18:0 ratio of greater than 100. The fatty acid profile can have a sum of C16:0 and C18:0 of 2.5% or less, or optionally, 2.2% or less.

The cell can overexpress both a KASII gene and a SAD gene. Optionally, the KASII gene encodes a mature KASII protein with at least 80, 85, 90, or 95% sequence identity to SEQ ID NO: 18 and/or the SAD gene encodes a mature SAD protein with at least 80, 85, 90, or 95% sequence identity to SEQ ID NO: 65. Optionally, the cell has a disruption of an endogenous FATA gene and/or an endogenous FAD2 gene. In some cases, the cell comprises a nucleic acid encoding an inhibitory RNA to down-regulate the expression of a desaturase. In some cases, the inhibitory RNA is a hairpin RNA that down regulates a FAD2 gene.

The cell can be a Eukaryotic microalgal cell; the oil has sterols with a sterol profile characterized by an excess of ergosterol over β-sitosterol and/or the presence of 22, 23-dihydrobrassicasterol, poriferasterol or clionasterol.

In an embodiment, a method includes cultivating the recombinant cell and extracting the oil from the cell. Optionally, the oil is used in a food product with at least one other edible ingredient or subjected to a chemical reaction.

In one embodiment, an oleaginous eukaryotic microalgal cell that produces a cell oil, the cell comprising an ablation (knock-out) of one or more alleles of an endogenous polynucleotide encoding a lysophosphatidic acid acyltransferase (LPAAT). In some embodiments, the cell comprises ablation of both alleles of an LPAAT. In some embodiments, the cell comprises ablation of an allele of an LPAAT identified as LPAAT1 or ablation of an LPAAT identified as LPAAT2. In some embodiments, the cell comprises ablation of both alleles of LPAAT1 and ablation of both alleles of LPAAT2.

In some embodiments, an oleaginous eukaryotic microalgal cell has both an ablation of an endogenous LPAAT and a recombinant nucleic acid that encodes one or more of an active LPCAT, PDCT, DAG-CPT, LPAAT and FAE. The LPCAT has at least 80, 85, 90 or 95% sequence identity to SEQ ID NO: 86, 87, 88, 89, 90, 91, or 92 or to the relevant portions of SEQ ID NO: 97, 98, 99, 100, 101, 102, or 103. The PDCT has at least 80, 85, 90 or 95% sequence identity to the relevant portions of SEQ ID NO: 93. The DAG-CPT has at least 80, 85, 90 or 95% sequence identity to the relevant portions of SEQ ID NO: 94, 95, or 96. The LPAAT has at least 80, 85, 90 or 95% sequence identity to the relevant portions of SEQ ID NO: 12, 16, 26, 27, 28, 29, 30, 31, 32, 33, 63, 82, or 83. The FAE has at least 80, 85, 90 or 95% sequence identity to the relevant portions of SEQ ID NO: 19, 20, 84, or 85.

In some embodiments, an oleaginous eukaryotic microalgal cell has both an ablation of an endogenous LPAAT and a first recombinant nucleic acid that encodes one or more of an active LPCAT, PDCT, DAG-CPT, and LPAAT and a second recombinant nucleic acid that encodes an active FAE.

In some embodiments, an oleaginous eukaryotic microalgal cell has both an ablation of an endogenous LPAAT and a recombinant nucleic acid that encodes one or more of an active LPCAT, PDCT, DAG-CPT, LPAAT and FAE and another recombinant nucleic acid that encodes an active sucrose invertase.

In some embodiments, the invention is an oil produced by a eukaryotic microalgal cell, the cell optionally of the genus Prototheca, the cell comprising an ablation of one or more alleles of an endogenous polynucleotide encoding LPAAT.

In other embodiments, the invention comprises an oil produced by a eukaryotic microalgal cell that has both an ablation of an endogenous LPAAT and a recombinant nucleic acid that encodes one or more of an active LPCAT, PDCT, DAG-CPT, LPAAT and FAE.

In some embodiments, the invention comprises an oil produced an oleaginous eukaryotic microalgal cell has both an ablation of an endogenous LPAAT and a first recombinant nucleic acid that encodes one or more of an active LPCAT, PDCT, DAG-CPT, and LPAAT and a second recombinant nucleic acid that encodes an active FAE.

In some embodiments, the oil comprises at least 10%, at least 15%, at least 20%, or at least 25% or higher C18:2. In other embodiments the oil comprises at least 5%, at least 10%, at least 20%, or at least 25% or higher C18:3. In some embodiments, the oil comprises at least 1%, at least 5%, at least 7%, or at least 10% or higher C20:1. In some embodiments, the oil comprises at least 1%, at least 5%, at least 7%, or at least 10% or higher C22:1.

In some embodiments, the oil comprises at least 10%, at least 15%, or at least 20% or higher of the combined amount of C20:1 and C22:1.

In some embodiments, the oil comprises less than 50%, less than 40%, less than 30%, or less than 20% or lower C18:1.

In some embodiments, an oleaginous eukaryotic microalgal cell that produces a cell oil, the cell comprising a recombinant nucleic acid that encodes one or more of an active enzymes selected from the group consisting of LPCAT, PDCT, DAG-CPT, LPAAT and FAE. In other embodiments, the cell comprises a second exogenous gene encoding an active sucrose invertase.

In an embodiment, an oleaginous eukaryotic microalgal cell produces a cell oil. The cell is optionally of the genus Prototheca and includes an first exogenous gene encoding an active enzyme of one of the following types:

(a) a lysophosphatidylcholine acyltransferase (LPCAT);
(b) a phosphatidylcholine diacylglycerol cholinephosphotransferase (PDCT); or
(c) CDP-choline:1,2-sn-diacylglycerol cholinephosphotransferase (DAG-CPT);
and optionally a second exogenous gene encoding
(d) a fatty acid elongase (FAE) active to increase the amount of C20:1 and/or C22:1 fatty acids in the oil.

In some embodiments methods of heterotrophically cultivating recombinant cells of the invention are provided. In some embodiments methods of cultivating recombinant cells heterotrophically and in the dark are provided. The cultivated cells can be dewatered and/or dried. Oil from the cultivated cells can be extracted by mechanical means. Oil from the cultivated cells can be extracted by the use of non-polar organic solvents such as hexane, heptane, pentane and the like. Alternatively methanol, ethanol, or other polar organic solvents may be used. When miscible solvents such as ethanol are used, salts such as NaCl may be used to “break” the emulsion between aqueous and organic phase.

In one aspect, the present invention is directed to an oil produced by an oleaginous eukaryotic microalgal cell as discussed above or herein.

In some embodiments, one or more chemical reactions are performed on the oil of the invention to produce a lubricant, fuel, or other useful products. In other embodiments, a food product is prepared by adding the oil of the invention to another edible food ingredient.

In one aspect, the present invention is directed to an oleaginous eukaryotic microalgal cell that produces a cell oil, in which the cell is optionally of the genus Prototheca, and the cell comprises an exogenous polynucleotide that encodes an active ketoacyl-CoA reductase, hydroxyacyl-CoA dehydratase, or enoyl-CoA reductase. In some embodiments, the exogenous polynucleotide has at least 80, 85, 90 or 95% sequence identity to SEQ ID NO: 144 and encodes an active ketoacyl-CoA reductase. In some embodiments, the exogenous polynucleotide has at least 80, 85, 90 or 95% sequence identity to SEQ ID NO: 143 and encodes an active hydroxyacyl-CoA dehydratase. In some embodiments, the exogenous polynucleotide has at least 80, 85, 90 or 95% sequence identity to the enoyl-CoA reductase encoding portion of SEQ ID NO: 142 and encodes an active enoyl-CoA reductase.

In some cases, the cell further comprises an exogenous nucleic acid encoding a lysophosphatidylcholine acyltransferase (LPCAT), a phosphatidylcholine diacylglycerol cholinephosphotransferase (PDCT), CDP-choline:1,2-sn-diacylglycerol cholinephosphotransferase (DAG-CPT), a lysophosphatidic acid acyltransferase (LPAAT) or a fatty acid elongase (FAE). In some cases, the cell further comprises an exogenous nucleic acid encoding an enzyme selected from the group consisting of a sucrose invertase and an alpha galactosidase. In some cases, the cell further comprises an exogenous nucleic acid that encodes a desaturase and/or a ketoacyl synthase. In some cases, the cell further comprises a disruption of an endogenous FATA gene. In some cases, the cell further comprises a disruption of an endogenous or FAD2 gene. In some embodiments, the cell further comprises a nucleic acid encoding an inhibitory RNA that down-regulates the expression of a desaturase.

In some embodiments, the cell oil comprises sterols with a sterol profile characterized by an excess of ergosterol over β-sitosterol and/or the presence of 22, 23-dihydrobrassicasterol, poriferasterol or clionasterol.

In one aspect, the present invention provides an oil produced by an oleaginous eukaryotic microalgal cell, in which the cell is optionally of the genus Prototheca, and the cell comprises an exogenous polynucleotide that encodes an active ketoacyl-CoA reductase, hydroxyacyl-CoA dehydratase, or enoyl-CoA reductase. In some cases, the exogenous polynucleotide has at least 80, 85, 90 or 95% sequence identity to SEQ ID NO: 144 and encodes an active ketoacyl-CoA reductase. In some cases, the exogenous polynucleotide has at least 80, 85, 90 or 95% sequence identity to SEQ ID NO: 143 and encodes an active hydroxyacyl-CoA dehydratase. In some cases, the exogenous polynucleotide has at least 80, 85, 90 or 95% sequence identity to the enoyl-CoA reductase encoding portion of SEQ ID NO: 142 and encodes an active enoyl-CoA reductase.

In some embodiments, the oil is produced by a cell that further comprises an exogenous nucleic acid encoding a lysophosphatidylcholine acyltransferase (LPCAT), a phosphatidylcholine diacylglycerol cholinephosphotransferase (PDCT), CDP-choline:1,2-sn-diacylglycerol cholinephosphotransferase (DAG-CPT), a lysophosphatidic acid acyltransferase (LPAAT) or a fatty acid elongase (FAE). In some cases, the cell further comprises and exogenous nucleic acid encoding an enzyme selected from the group consisting of a sucrose invertase and an alpha galactosidase.

In some cases, the oil comprises at least 10% C18:2. In some cases, the oil comprises at least 15% C18:2. In some cases, the oil comprises at least 1% C18:3. In some cases, the oil comprises at least 5% C18:3. In some cases, the oil comprises at least 10% C18:3. In some cases, the oil comprises at least 1% C20:1. In some cases, the oil comprises at least 5% C20:1. In some cases, the oil comprises at least 7% C20:1. In some cases, the oil comprises at least 1% C22:1. In some cases, the oil comprises at least 5% C22:1. In some cases, the oil comprises at least 7% C22:1. In some embodiments, the oil comprises sterols with a sterol profile characterized by an excess of ergosterol over β-sitosterol and/or the presence of 22, 23-dihydrobrassicasterol, poriferasterol or clionasterol.

In one aspect, the present invention is directed to a cell of the genera Prototheca or Chlorella that produces a cell oil, wherein the cell comprises an exogenous polynucleotide that replaces an endogenous regulatory element of an endogenous gene. In some cases, the cell is a Prototheca cell. In some cases, the cell is a Prototheca moriformis cell.

In some embodiments, the endogenous regulatory element is a promoter that controls the expression of an endogenous acetyl-CoA carboxylase. In some cases, the exogenous polynucleotide is a Prototheca moriformis AMT03 promoter.

In some cases, the cell further comprises an exogenous nucleic acid that encodes an active ketoacyl-CoA reductase, hydroxyacyl-CoA dehydratase, or enoyl-CoA reductase. In some embodiments, the exogenous nucleic acid has at least 80, 85, 90 or 95% sequence identity to SEQ ID NO: 144 and encodes an active ketoacyl-CoA reductase. In some embodiments, the exogenous nucleic acid has at least 80, 85, 90 or 95% sequence identity to SEQ ID NO: 143 and encodes an active hydroxyacyl-CoA dehydratase. In some embodiments, the exogenous nucleic acid has at least 80, 85, 90 or 95% sequence identity to the enoyl-CoA reductase encoding portion of SEQ ID NO: 142 and encodes an active enoyl-CoA reductase.

In some cases, the cell further comprises an exogenous nucleic acid encoding a lysophosphatidylcholine acyltransferase (LPCAT), a phosphatidylcholine diacylglycerol cholinephosphotransferase (PDCT), CDP-choline:1,2-sn-diacylglycerol cholinephosphotransferase (DAG-CPT), a lysophosphatidic acid acyltransferase (LPAAT) or a fatty acid elongase (FAE). In some cases, the cell further comprises an exogenous nucleic acid that encodes a desaturase and/or a ketoacyl synthase. In some cases, the cell further comprises a disruption of an endogenous FATA gene. In some cases, the cell further comprises a disruption of an endogenous or FAD2 gene. In some cases, the cell further comprises a nucleic acid encoding an inhibitory RNA that down-regulates the expression of a desaturase.

In some embodiments, the cell oil comprises sterols with a sterol profile characterized by an excess of ergosterol over β-sitosterol and/or the presence of 22, 23-dihydrobrassicasterol, poriferasterol or clionasterol.

In one aspect, the present invention provides an oil produced by any one of the cells discussed above or herein.

In one aspect, the present invention provides a method comprising (a) cultivating a cell as discussed above or herein to produce an oil, and (b) extracting the oil from the cell.

In one aspect, the present invention provides a method of preparing a composition comprising subjecting the oil discussed above or herein to a chemical reaction.

In one aspect, the present invention provides a method of preparing a food product comprising adding the oil discussed above or herein to another edible ingredient.

In one aspect, the present invention provides a polynucleotide with at least 80, 85, 90 or 95% sequence identity to SEQ ID NO: 144. In some cases, the polynucleotide comprises the nucleotide sequence of SEQ ID NO: 144.

In one aspect, the present invention provides a polynucleotide with at least 80, 85, 90 or 95% sequence identity to SEQ ID NO: 143. In some cases, the polynucleotide comprises the nucleotide sequence of SEQ ID NO: 143.

In one aspect, the present invention provides a polynucleotide with at least 80, 85, 90 or 95% sequence identity to nucleotides 4884 to 5816 of SEQ ID NO: 142. In some cases, the polynucleotide comprises the nucleotide sequence of nucleotides 4884 to 5816 of SEQ ID NO: 142.

In one aspect, the present invention provides a ketoacyl-CoA reductase (KCR) encoded by the nucleotide sequence of SEQ ID NO: 144. In some cases, the KCR is encoded by a polynucleotide with at least 80, 85, 90 or 95% sequence identity to SEQ ID NO: 144.

In one aspect, the present invention provides a hydroxylacyl-CoA dehydratase (HACD) encoded by the nucleotide sequence of SEQ ID NO: 143. In some cases, the HACD is encoded by a polynucleotide with at least 80, 85, 90 or 95% sequence identity to SEQ ID NO: 143.

In one aspect, the present invention provides an enoyl-CoA reductase (ECR) encoded by the nucleotide sequence of nucleotides 4884 to 5816 of SEQ ID NO: 142. In some cases, the ECR is encoded by a polynucleotide with at least 80, 85, 90 or 95% sequence identity to nucleotides 4884 to 5816 of SEQ ID NO: 142.

In various embodiments of the invention, two or more features discussed above or herein can be combined together.

### BRIEF DESCRIPTION OF THE DRAWINGS

FIG. 1 shows the total saturated fatty acid levels of S8188 in 15-L fed-batch fermentation runs 140558F22 and 140574F24.

FIG. 2 shows the percent saturates produced from various cell lines discussed in Example 17. “MCB” refers to the master cell bank, and “WCB” refers to the working cell bank. Strains S8695 and S8696, when cultivated in liquid culture media, had total saturates of about 3.6% and 3.75%, respectively.

FIG. 3 shows the alignment of the amino acid sequences of P. morformis and plant ketoacyl-CoA reductase proteins.

FIG. 4 shows the alignment of the amino acid sequences of P. morformis and plant hydroxyacyl-CoA dehydratase proteins.

FIG. 5 shows the alignment of the amino acid sequences of P. morformis and plant enoyl-CoA reductase proteins.

FIGS. 6A and 6B show the alignment of the amino acid sequences of the two alleles of P. morformis acetyl-CoA carboxylase proteins, PmACCase 1-1 and PmACCase1-2

### DETAILED DESCRIPTION OF THE INVENTION

### I. Definitions

An “allele” refers to a copy of a gene where an organism has multiple similar or identical gene copies, even if on the same chromosome. An allele may encode the same or similar protein.

In connection with two fatty acids in a fatty acid profile, “balanced” shall mean that the two fatty acids are within a specified percentage of their mean area percent. Thus, for fatty acid a in x % abundance and fatty acid b in y % abundance, the fatty acids are “balanced to within z %” if |x−((x+y)/2)| and |y−((x+y)/2)| are ≦100(z).

A “cell oil” or “cell fat” shall mean a predominantly triglyceride oil obtained from an organism, where the oil has not undergone blending with another natural or synthetic oil, or fractionation so as to substantially alter the fatty acid profile of the triglyceride. In connection with an oil comprising triglycerides of a particular regiospecificity, the cell oil or cell fat has not been subjected to interesterification or other synthetic process to obtain that regiospecific triglyceride profile, rather the regiospecificity is produced naturally, by a cell or population of cells. For a cell oil produced by a cell, the sterol profile of oil is generally determined by the sterols produced by the cell, not by artificial reconstitution of the oil by adding sterols in order to mimic the cell oil. In connection with a cell oil or cell fat, and as used generally throughout the present disclosure, the terms oil and fat are used interchangeably, except where otherwise noted. Thus, an “oil” or a “fat” can be liquid, solid, or partially solid at room temperature, depending on the makeup of the substance and other conditions. Here, the term “fractionation” means removing material from the oil in a way that changes its fatty acid profile relative to the profile produced by the organism, however accomplished. The terms “cell oil” and “cell fat” encompass such oils obtained from an organism, where the oil has undergone minimal processing, including refining, bleaching and/or degumming, which does not substantially change its triglyceride profile. A cell oil can also be a “noninteresterified cell oil”, which means that the cell oil has not undergone a process in which fatty acids have been redistributed in their acyl linkages to glycerol and remain essentially in the same configuration as when recovered from the organism.

“Exogenous gene” shall mean a nucleic acid that codes for the expression of an RNA and/or protein that has been introduced into a cell (e.g. by transformation/transfection), and is also referred to as a “transgene”. A cell comprising an exogenous gene may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome (nuclear or plastid) or as an episomal molecule.

“FADc”, also referred to as “FAD2” is a gene encoding a delta-12 fatty acid desaturase.

“Fatty acids” shall mean free fatty acids, fatty acid salts, or fatty acyl moieties in a glycerolipid. It will be understood that fatty acyl groups of glycerolipids can be described in terms of the carboxylic acid or anion of a carboxylic acid that is produced when the triglyceride is hydrolyzed or saponified.

“Fixed carbon source” is a molecule(s) containing carbon, typically an organic molecule that is present at ambient temperature and pressure in solid or liquid form in a culture media that can be utilized by a microorganism cultured therein. Accordingly, carbon dioxide is not a fixed carbon source.

“In operable linkage” is a functional linkage between two nucleic acid sequences, such a control sequence (typically a promoter) and the linked sequence (typically a sequence that encodes a protein, also called a coding sequence). A promoter is in operable linkage with an exogenous gene if it can mediate transcription of the gene.

“Microalgae” are eukaryotic microbial organisms that contain a chloroplast or other plastid, and optionally that is capable of performing photosynthesis, or a prokaryotic microbial organism capable of performing photosynthesis. Microalgae include obligate photoautotrophs, which cannot metabolize a fixed carbon source as energy, as well as heterotrophs, which can live solely off of a fixed carbon source. Microalgae include unicellular organisms that separate from sister cells shortly after cell division, such as Chlamydomonas, as well as microbes such as, for example, Volvox, which is a simple multicellular photosynthetic microbe of two distinct cell types. Microalgae include cells such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis, such as certain dinoflagellate algae species and species of the genus Prototheca.

In connection with fatty acid length, “mid-chain” shall mean C8 to C16 fatty acids.

In connection with a recombinant cell, the term “knockdown” refers to a gene that has been partially suppressed (e.g., by about 1-95%) in terms of the production or activity of a protein encoded by the gene.

Also, in connection with a recombinant cell, the term “knockout” refers to a gene that has been completely or nearly completely (e.g., >95%) suppressed in terms of the production or activity of a protein encoded by the gene. Knockouts can be prepared by ablating the gene by homologous recombination of a nucleic acid sequence into a coding sequence, gene deletion, mutation or other method. When homologous recombination is performed, the nucleic acid that is inserted (“knocked-in”) can be a sequence that encodes an exogenous gene of interest or a sequence that does not encode for a gene of interest.

An “oleaginous” cell is a cell capable of producing at least 20% lipid by dry cell weight, naturally or through recombinant or classical strain improvement. An “oleaginous microbe” or “oleaginous microorganism” is a microbe, including a microalga that is oleaginous (especially eukaryotic microalgae that store lipid). An oleaginous cell also encompasses a cell that has had some or all of its lipid or other content removed, and both live and dead cells.

An “ordered oil” or “ordered fat” is one that forms crystals that are primarily of a given polymorphic structure. For example, an ordered oil or ordered fat can have crystals that are greater than 50%, 60%, 70%, 80%, or 90% of the 13 or (3′ polymorphic form.

In connection with a cell oil, a “profile” is the distribution of particular species or triglycerides or fatty acyl groups within the oil. A “fatty acid profile” is the distribution of fatty acyl groups in the triglycerides of the oil without reference to attachment to a glycerol backbone. Fatty acid profiles are typically determined by conversion to a fatty acid methyl ester (FAME), followed by gas chromatography (GC) analysis with flame ionization detection (FID), as in Example 1. The fatty acid profile can be expressed as one or more percent of a fatty acid in the total fatty acid signal determined from the area under the curve for that fatty acid. FAME-GC-FID measurement approximate weight percentages of the fatty acids. A “sn-2 profile” is the distribution of fatty acids found at the sn-2 position of the triacylglycerides in the oil. A “regiospecific profile” is the distribution of triglycerides with reference to the positioning of acyl group attachment to the glycerol backbone without reference to stereospecificity. In other words, a regiospecific profile describes acyl group attachment at sn-1/3 vs. sn-2. Thus, in a regiospecific profile, POS (palmitate-oleate-stearate) and SOP (stearate-oleate-palmitate) are treated identically. A “stereospecific profile” describes the attachment of acyl groups at sn-1, sn-2 and sn-3. Unless otherwise indicated, triglycerides such as SOP and POS are to be considered equivalent. A “TAG profile” is the distribution of fatty acids found in the triglycerides with reference to connection to the glycerol backbone, but without reference to the regiospecific nature of the connections. Thus, in a TAG profile, the percent of SSO in the oil is the sum of SSO and SOS, while in a regiospecific profile, the percent of SSO is calculated without inclusion of SOS species in the oil. In contrast to the weight percentages of the FAME-GC-FID analysis, triglyceride percentages are typically given as mole percentages; that is the percent of a given TAG molecule in a TAG mixture.

The term “percent sequence identity,” in the context of two or more amino acid or nucleic acid sequences, refers to two or more sequences or subsequences that are the same or have a specified percentage of amino acid residues or nucleotides that are the same, when compared and aligned for maximum correspondence, as measured using a sequence comparison algorithm or by visual inspection. For sequence comparison to determine percent nucleotide or amino acid identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted using the NCBI BLAST software (ncbi.nlm.nih.gov/BLAST/) set to default parameters. For example, to compare two nucleic acid sequences, one may use blastn with the “BLAST 2 Sequences” tool Version 2.0.12 (Apr. 21, 2000) set at the following default parameters: Matrix: BLOSUM62; Reward for match: 1; Penalty for mismatch: −2; Open Gap: 5 and Extension Gap: 2 penalties; Gap x drop-off: 50; Expect: 10; Word Size: 11; Filter: on. For a pairwise comparison of two amino acid sequences, one may use the “BLAST 2 Sequences” tool Version 2.0.12 (Apr. 21, 2000) with blastp set, for example, at the following default parameters: Matrix: BLOSUM62; Open Gap: 11 and Extension Gap: 1 penalties; Gap x drop-off 50; Expect: 10; Word Size: 3; Filter: on.

“Recombinant” is a cell, nucleic acid, protein or vector that has been modified due to the introduction of an exogenous nucleic acid or the alteration of a native nucleic acid. Thus, e.g., recombinant cells can express genes that are not found within the native (non-recombinant) form of the cell or express native genes differently than those genes are expressed by a non-recombinant cell. Recombinant cells can, without limitation, include recombinant nucleic acids that encode for a gene product or for suppression elements such as mutations, knockouts, antisense, interfering RNA (RNAi) or dsRNA that reduce the levels of active gene product in a cell. A “recombinant nucleic acid” is a nucleic acid originally formed in vitro, in general, by the manipulation of nucleic acid, e.g., using polymerases, ligases, exonucleases, and endonucleases, using chemical synthesis, or otherwise is in a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Similarly, a “recombinant protein” is a protein made using recombinant techniques, i.e., through the expression of a recombinant nucleic acid.

The terms “triglyceride”, “triacylglyceride” and “TAG” are used interchangeably as is known in the art.

### II. General

Illustrative embodiments of the present invention feature oleaginous cells that produce altered fatty acid profiles and/or altered regiospecific distribution of fatty acids in glycerolipids, and products produced from the cells. Examples of oleaginous cells include microbial cells having a type II fatty acid biosynthetic pathway, including plastidic oleaginous cells such as those of oleaginous algae and, where applicable, oil producing cells of higher plants including but not limited to commercial oilseed crops such as soy, corn, rapeseed/canola, cotton, flax, sunflower, safflower and peanut. Other specific examples of cells include heterotrophic or obligate heterotrophic microalgae of the phylum Chlorophtya, the class Trebouxiophytae, the order Chlorellales, or the family Chlorellacae. Examples of oleaginous microalgae and method of cultivation are also provided in Published PCT Patent Applications WO2008/151149, WO2010/06032, WO2011/150410, and WO2011/150411, including species of Chlorella and Prototheca, a genus comprising obligate heterotrophs. The oleaginous cells can be, for example, capable of producing 25, 30, 40, 50, 60, 70, 80, 85, or about 90% oil by cell weight, ±5%. Optionally, the oils produced can be low in highly unsaturated fatty acids such as DHA or EPA fatty acids. For example, the oils can comprise less than 5%, 2%, or 1% DHA and/or EPA. The above-mentioned publications also disclose methods for cultivating such cells and extracting oil, especially from microalgal cells; such methods are applicable to the cells disclosed herein and incorporated by reference for these teachings. When microalgal cells are used they can be cultivated autotrophically (unless an obligate heterotroph) or in the dark using a sugar (e.g., glucose, fructose and/or sucrose) In any of the embodiments described herein, the cells can be heterotrophic cells comprising an exogenous invertase gene so as to allow the cells to produce oil from a sucrose feedstock. Alternately, or in addition, the cells can metabolize xylose from cellulosic feedstocks. For example, the cells can be genetically engineered to express one or more xylose metabolism genes such as those encoding an active xylose transporter, a xylulose-5-phosphate transporter, a xylose isomerase, a xylulokinase, a xylitol dehydrogenase and a xylose reductase. See WO2012/154626, “GENETICALLY ENGINEERED MICROORGANISMS THAT METABOLIZE XYLOSE”, published Nov. 15, 2012, including disclosure of genetically engineered Prototheca strains that utilize xylose.

The oleaginous cells may, optionally, be cultivated in a bioreactor/fermenter. For example, heterotrophic oleaginous microalgal cells can be cultivated on a sugar-containing nutrient broth. Optionally, cultivation can proceed in two stages: a seed stage and a lipid-production stage. In the seed stage, the number of cells is increased from a starter culture. Thus, the seed stage(s) typically includes a nutrient rich, nitrogen replete, media designed to encourage rapid cell division. After the seed stage(s), the cells may be fed sugar under nutrient-limiting (e.g. nitrogen sparse) conditions so that the sugar will be converted into triglycerides. As used herein, “standard lipid production conditions” means that the culture conditions are nitrogen limiting. Sugar and other nutrients can be added during the fermentation but no additional nitrogen is added. The cells will consume all or nearly all of the nitrogen present, but no additional nitrogen is provided. For example, the rate of cell division in the lipid-production stage can be decreased by 50%, 80% or more relative to the seed stage. Additionally, variation in the media between the seed stage and the lipid-production stage can induce the recombinant cell to express different lipid-synthesis genes and thereby alter the triglycerides being produced. For example, as discussed below, nitrogen and/or pH sensitive promoters can be placed in front of endogenous or exogenous genes. This is especially useful when an oil is to be produced in the lipid-production phase that does not support optimal growth of the cells in the seed stage.

The oleaginous cells express one or more exogenous genes encoding fatty acid biosynthesis enzymes. As a result, some embodiments feature cell oils that were not obtainable from a non-plant or non-seed oil, or not obtainable at all.

The oleaginous cells (optionally microalgal cells) can be improved via classical strain improvement techniques such as UV and/or chemical mutagenesis followed by screening or selection under environmental conditions, including selection on a chemical or biochemical toxin. For example the cells can be selected on a fatty acid synthesis inhibitor, a sugar metabolism inhibitor, or an herbicide. As a result of the selection, strains can be obtained with increased yield on sugar, increased oil production (e.g., as a percent of cell volume, dry weight, or liter of cell culture), or improved fatty acid or TAG profile. Co-owned U.S. application 60/141,167 filed on 31 Mar. 2015 describes methods for classically mutagenizing oleaginous cells.

For example, the cells can be selected on one or more of 1,2-Cyclohexanedione; 19-Norethindone acetate; 2,2-dichloropropionic acid; 2,4,5-trichlorophenoxyacetic acid; 2,4,5-trichlorophenoxyacetic acid, methyl ester; 2,4-dichlorophenoxyacetic acid; 2,4-dichlorophenoxyacetic acid, butyl ester; 2,4-dichlorophenoxyacetic acid, isooctyl ester; 2,4-dichlorophenoxyacetic acid, methyl ester; 2,4-dichlorophenoxybutyric acid; 2,4-dichlorophenoxybutyric acid, methyl ester; 2,6-dichlorobenzonitrile; 2-deoxyglucose; 5-Tetradecyloxy-w-furoic acid; A-922500; acetochlor; alachlor; ametryn; amphotericin; atrazine; benfluralin; bensulide; bentazon; bromacil; bromoxynil; Cafenstrole; carbonyl cyanide m-chlorophenyl hydrazone (CCCP); carbonyl cyanide-p-trifluoromethoxyphenylhydrazone (FCCP); cerulenin; chlorpropham; chlorsulfuron; clofibric acid; clopyralid; colchicine; cycloate; cyclohexamide; C75; DACTHAL (dimethyl tetrachloroterephthalate); dicamba; dichloroprop ((R)-2-(2,4-dichlorophenoxy)propanoic acid); Diflufenican; dihyrojasmonic acid, methyl ester; diquat; diuron; dimethylsulfoxide; Epigallocatechin gallate (EGCG); endothall; ethalfluralin; ethanol; ethofumesate; Fenoxaprop-p-ethyl; Fluazifop-p-Butyl; fluometuron; fomasefen; foramsulfuron; gibberellic acid; glufosinate ammonium; glyphosate; haloxyfop; hexazinone; imazaquin; isoxaben; Lipase inhibitor THL ((−)-Tetrahydrolipstatin); malonic acid; MCPA (2-methyl-4-chlorophenoxyacetic acid); MCPB (4-(4-chloro-o-tolyloxy)butyric acid); mesotrione; methyl dihydrojasmonate; metolachlor; metribuzin; Mildronate; molinate; naptalam; norharman; orlistat; oxadiazon; oxyfluorfen; paraquat; pendimethalin; pentachlorophenol; PF-04620110; phenethyl alcohol; phenmedipham; picloram; Platencin; Platensimycin; prometon; prometryn; pronamide; propachlor; propanil; propazine; pyrazon; Quizalofop-p-ethyl; s-ethyl dipropylthiocarbamate (EPTC); s,s,s-tributylphosphorotrithioate; salicylhydroxamic acid; sesamol; siduron; sodium methane arsenate; simazine; T-863 (DGAT inhibitor); tebuthiuron; terbacil; thiobencarb; tralkoxydim; triallate; triclopyr; triclosan; trifluralin; and vulpinic acid.

The oleaginous cells produce a storage oil, which is primarily triacylglyceride and may be stored in storage bodies of the cell. A raw oil may be obtained from the cells by disrupting the cells and isolating the oil. The raw oil may comprise sterols produced by the cells. WO2008/151149, WO2010/06032, WO2011/150410, and WO2011/1504 disclose heterotrophic cultivation and oil isolation techniques for oleaginous microalgae. For example, oil may be obtained by providing or cultivating, drying and pressing the cells. The oils produced may be refined, bleached and deodorized (RBD) as known in the art or as described in WO2010/120939. The raw or RBD oils may be used in a variety of food, chemical, and industrial products or processes. Even after such processing, the oil may retain a sterol profile characteristic of the source. Microalgal sterol profiles are disclosed below. See especially Section XIII of this patent application. After recovery of the oil, a valuable residual biomass remains. Uses for the residual biomass include the production of paper, plastics, absorbents, adsorbents, drilling fluids, as animal feed, for human nutrition, or for fertilizer.

The nucleic acids of the invention may contain control sequences upstream and downstream in operable linkage with the gene of interest, including LPAAT, LPCAT, FAE, PDCT, DAG-CPT, and other lipid biosynthetic pathway genes as discussed herein. These control sequences include promoters, targeting sequences, untranslated sequences and other control elements.

The nucleic acids of the invention can be codon optimized for expression in a target host cell (e.g., using the codon usage tables of Tables 1 and 2.) For example, at least 60, 65, 70, 75, 80, 85, 90, 95 or 100% of the codons used can be the most preferred codon according to Table 1 or 2. Alternately, at least 60, 65, 70, 75, 80, 85, 90, 95 or 100% of the codons used can be the first or second most preferred codon according to Table 1 or 2. Preferred codons for Prototheca strains and for Chlorella protothecoides are shown below in Tables 1 and 2, respectively.

TABLE 1
Preferred codon usage in Prototheca strains.

| | | | | | |
|---|---|---|---|---|---|
| Ala | GCG | 345 (0.36) | Asn | AAT | 8 (0.04) |
| | GCA | 66 (0.07) | | AAC | 201 (0.96) |
| | GCT | 101 (0.11) | | | |
| | GCC | 442 (0.46) | Pro | CCG | 161 (0.29) |
| | | | | CCA | 49 (0.09) |
| Cys | TGT | 12 (0.10) | | CCT | 71 (0.13) |
| | TGC | 105 (0.90) | | CCC | 267 (0.49) |
| Asp | GAT | 43 (0.12) | Gln | CAG | 226 (0.82) |
| | GAC | 316 (0.88) | | CAA | 48 (0.18) |
| Glu | GAG | 377 (0.96) | Arg | AGG | 33 (0.06) |
| | GAA | 14 (0.04) | | AGA | 14 (0.02) |
| | | | | CGG | 102 (0.18) |
| Phe | TTT | 89 (0.29) | | CGA | 49 (0.08) |
| | TTC | 216 (0.71) | | CGT | 51 (0.09) |
| | | | | CGC | 331 (0.57) |
| Gly | GGG | 92 (0.12) | | | |
| | GGA | 56 (0.07) | Ser | AGT | 16 (0.03) |
| | GGT | 76 (0.10) | | AGC | 123 (0.22) |
| | GGC | 559 (0.71) | | TCG | 152 (0.28) |
| | | | | TCA | 31 (0.06) |
| His | CAT | 42 (0.21) | | TCT | 55 (0.10) |
| | CAC | 154 (0.79) | | TCC | 173 (0.31) |
| Ile | ATA | 4 (0.01) | Thr | ACG | 184 (0.38) |
| | ATT | 30 (0.08) | | ACA | 24 (0.05) |
| | ATC | 338 (0.91) | | ACT | 21 (0.05) |
| | | | | ACC | 249 (0.52) |
| Lys | AAG | 284 (0.98) | | | |
| | AAA | 7 (0.02) | Val | GTG | 308 (0.50) |
| | | | | GTA | 9 (0.01) |
| Leu | TTG | 26 (0.04) | | GTT | 35 (0.06) |
| | TTA | 3 (0.00) | | GTC | 262 (0.43) |
| | CTG | 447 (0.61) | | | |
| | CTA | 20 (0.03) | Trp | TGG | 107 (1.00) |
| | CTT | 45 (0.06) | | | |
| | CTC | 190 (0.26) | Tyr | TAT | 10 (0.05) |
| | | | | TAC | 180 (0.95) |
| Met | ATG | 191 (1.00) | | | |

| | |
|---|---|
| Stop | TGA/TAG/TAA |

TABLE 2
Preferred codon usage in Chlorella protothecoides.

| | | | |
|---|---|---|---|
| TTC (Phe) | TAC (Tyr) | TGC (Cys) | TGA (Stop) |
| TGG (Trp) | CCC (Pro) | CAC (His) | CGC (Arg) |
| CTG (Leu) | CAG (Gln) | ATC (Ile) | ACC (Thr) |
| GAC (Asp) | TCC (Ser) | ATG (Met) | AAG (Lys) |
| GCC (Ala) | AAC (Asn) | GGC (Gly) | GTG (Val) |
| GAG (Glu) | | | |

The cell oils of this invention can be distinguished from conventional vegetable or animal triacylglycerol sources in that the sterol profile will be indicative of the host organism as distinguishable from the conventional source. Conventional sources of oil include soy, corn, sunflower, safflower, palm, palm kernel, coconut, cottonseed, canola, rape, peanut, olive, flax, tallow, lard, cocoa, shea, mango, sal, illipe, kokum, and allanblackia. See section XIII of this disclosure for a discussion of microalgal sterols.

TABLE 3
The fatty acid profiles of some commercial oilseed strains.

| Common Food Oils* | C12:0 | C14:0 | C16:0 | C16:1 | C18:0 | C18:1 | C18:2 | C18:3 |
|---|---|---|---|---|---|---|---|---|
| Corn oil (Zea mays) | | <1.0 | 8.0-19.0 | <0.5 | 0.5-4.0 | 19-50 | 38-65 | <2.0 |
| Cottonseed oil (Gossypium barbadense) | <0.1 | 0.5-2.0 | 17-29 | <1.5 | 1.0-4.0 | 13-44 | 40-63 | 0.1-2.1 |
| Canola (Brassica rapa, B. napus, B. juncea) | <0.1 | <0.2 | <6.0 | <1.0 | <2.5 | >50 | <40 | <14 |
| Olive (Olea europea) | | <0.1 | 6.5-20.0 | ≦3.5 | 0.5-5.0 | 56-85 | 3.5-20.0 | ≦1.2 |
| Peanut (Arachis hypogaea) | <0.1 | <0.2 | 7.0-16.0 | <1.0 | 1.3-6.5 | 35-72 | 13.0-43 | <0.6 |
| Palm (Elaeis guineensis) | | 0.5-5.9 | 32.0-47.0 | | 2.0-8.0 | 34-44 | 7.2-12.0 | |
| Safflower (Carthamus tinctorus) | <0.1 | <1.0 | 2.0-10.0 | <0.5 | 1.0-10.0 | 7.0-16.0 | 72-81 | <1.5 |
| Sunflower (Helianthus annus) | <0.1 | <0.5 | 3.0-10.0 | <1.0 | 1.0-10.0 | 14-65 | 20-75 | <0.5 |
| Soybean (Glycine max) | <0.1 | <0.5 | 7.0-12.0 | <0.5 | 2.0-5.5 | 19-30 | 48-65 | 5.0-10.0 |
| Solin-Flax (Linum usitatissimum) | <0.1 | <0.5 | 2.0-9.0 | <0.5 | 2.0-5.0 | 8.0-60 | 40-80 | <5.0 |

*Unless otherwise indicated, data taken from the U.S. Pharacopeia's Food and Chemicals Codex, 7th Ed. 2010-2011**

Where a fatty acid profile of a triglyceride (also referred to as a “triacylglyceride” or “TAG”) cell oil is given here, it will be understood that this refers to a nonfractionated sample of the storage oil extracted from the cell analyzed under conditions in which phospholipids have been removed or with an analysis method that is substantially insensitive to the fatty acids of the phospholipids (e.g. using chromatography and mass spectrometry). The oil may be subjected to an RBD process to remove phospholipids, free fatty acids and odors yet have only minor or negligible changes to the fatty acid profile of the triglycerides in the oil. Because the cells are oleaginous, in some cases the storage oil will constitute the bulk of all the TAGs in the cell. Example 1 below gives analytical methods for determining TAG fatty acid composition and regiospecific structure.

Broadly categorized, certain embodiments of the invention include (i) recombinant oleaginous cells that comprise an ablation of one or two or all alleles of an endogenous polynucleotide, including polynucleotides encoding lysophosphatidic acid acyltransferase (LPAAT) or (ii) cells that produce oils having low concentrations of polyunsaturated fatty acids, including cells that are auxotrophic for unsaturated fatty acids; (iii) cells producing oils having high concentrations of particular fatty acids due to expression of one or more exogenous genes encoding enzymes that transfer fatty acids to glycerol or a glycerol ester; (iv) cells producing regiospecific oils, (v) genetic constructs or cells encoding a an LPAAT, a lysophosphatidylcholine acyltransferase (LPCAT), a phosphatidylcholine diacylglycerol cholinephosphotransferase (PDCT), diacylglycerol cholinephosphotransferase (DAG-CPT) or fatty acyl elongase (FAE), (vi) cells producing low levels of saturated fatty acids and/or high levels of C18:1, C18:2, C18:3, C20:1 or C22:1, (vii) and other inventions related to producing cell oils with altered profiles. The embodiments also encompass the oils made by such cells, the residual biomass from such cells after oil extraction, oleochemicals, fuels and food products made from the oils and methods of cultivating the cells.

In any of the embodiments below, the cells used are optionally cells having a type II fatty acid biosynthetic pathway such as microalgal cells including heterotrophic or obligate heterotrophic microalgal cells, including cells classified as Chlorophyta, Treboindophyceae, Chlorellales, Chlorellaceae, or Chlorophyceae, or cells engineered to have a type II fatty acid biosynthetic pathway using the tools of synthetic biology (i.e., transplanting the genetic machinery for a type II fatty acid biosynthesis into an organism lacking such a pathway). Use of a host cell with a type II pathway avoids the potential for non-interaction between an exogenous acyl-ACP thioesterase or other ACP-binding enzyme and the multienzyme complex of type I cellular machinery. In specific embodiments, the cell is of the species Prototheca moriformis, Prototheca krugani, Prototheca stagnora or Prototheca zopfii or has a 23S rRNA sequence with at least 65, 70, 75, 80, 85, 90 or 95% nucleotide identity SEQ ID NO: 25. By cultivating in the dark or using an obligate heterotroph, the cell oil produced can be low in chlorophyll or other colorants. For example, the cell oil can have less than 100, 50, 10, 5, 1, 0.0.5 ppm of chlorophyll without substantial purification.

The stable carbon isotope value δ13C is an expression of the ratio of ¹³C/¹²C relative to a standard (e.g. PDB, carbonite of fossil skeleton of Belemnite americana from Peedee formation of South Carolina). The stable carbon isotope value δ13C (%) of the oils can be related to the δ13C value of the feedstock used. In some embodiments the oils are derived from oleaginous organisms heterotrophically grown on sugar derived from a C4 plant such as corn or sugarcane. In some embodiments the δ13C (%) of the oil is from −10 to −17% from −13 to −16%.

In specific embodiments and examples discussed below, one or more fatty acid synthesis genes (e.g., encoding an acyl-ACP thioesterase, a keto-acyl ACP synthase, an LPAAT, an LPCAT, a PDCT, a DAG-CPT, an FAE a stearoyl ACP desaturase, or others described herein) is incorporated into a microalga. It has been found that for certain microalga, a plant fatty acid synthesis gene product is functional in the absence of the corresponding plant acyl carrier protein (ACP), even when the gene product is an enzyme, such as an acyl-ACP thioesterase, that requires binding of ACP to function. Thus, optionally, the microalgal cells can utilize such genes to make a desired oil without co-expression of the plant ACP gene.

For the various embodiments of recombinant cells comprising exogenous genes or combinations of genes, it is contemplated that substitution of those genes with genes having 60, 70, 80, 85, 90, 91, 92, 93, 94, 95, 96, 97, 98, or 99% nucleic acid sequence identity can give similar results, as can substitution of genes encoding proteins having 60, 70, 80, 85, 90, 91, 92, 93, 94, 95, 95.5, 96, 96.5, 97, 97.5, 98, 98.5, 99 or 99.5% amino acid sequence identity. Likewise, for novel regulatory elements, it is contemplated that substitution of those nucleic acids with nucleic acids having 60, 70, 80, 85, 90, 91, 92, 93, 94, 95, 96, 97, 98, or 99% nucleic acid can be efficacious. In the various embodiments, it will be understood that sequences that are not necessary for function (e.g. FLAG® tags or inserted restriction sites) can often be omitted in use or ignored in comparing genes, proteins and variants.

Although discovered using or exemplified with microalgae, the novel genes and gene combinations reported here can be used in higher plants using techniques that are well known in the art. For example, the use of exogenous lipid metabolism genes in higher plants is described in U.S. Pat. Nos. 6,028,247, 5,850,022, 5,639,790, 5,455,167, 5,512,482, and 5,298,421 disclose higher plants with exogenous acyl-ACP thioesterases. WO2009129582 and WO1995027791 disclose cloning of LPAAT in plants. FAD2 suppression in higher plants is taught in WO 2013112578, and WO 2008006171.

As described in Example 7, transcript profiling was used to discover promoters that modulate expression in response to low nitrogen conditions. The promoters are useful to selectively express various genes and to alter the fatty acid composition of microbial oils. In accordance with an embodiment, there are non-natural constructs comprising a heterologous promoter and a gene, wherein the promoter comprises at least 60, 65, 70, 75, 80, 85, 90, or 95% sequence identity to any of the promoters of Example 7 (e.g., SEQ ID NOs: 43-58) and the gene is differentially expressed under low vs. high nitrogen conditions. Optionally, the expression is less pH sensitive than for the AMT03 promoter. For example, the promoters can be placed in front of a FAD2 gene in a linoleic acid auxotroph to produce an oil with less than 5, 4, 3, 2, or 1% linoleic acid after culturing under high, then low nitrogen conditions.

### III. Ablation (Knock Out) of LPAAT and/or FATA

In an embodiment, the cell is genetically engineered so that one, two or all alleles of a lipid pathway gene are knocked out. In an embodiment, the lipid pathway gene is an LPAAT gene. Alternately, the amount or activity of the gene products of the alleles is knocked down, for example by inhibitory RNA technologies including RNAi, siRNA, miRNA, dsRNA, antisense, and hairpin RNA techniques. When one allele of the lipid pathway gene is knocked out, a corresponding decrease in the enzymatic activity is observed. When all alleles of the lipid pathway gene are knocked out or sufficiently inhibited an auxotroph is created. A first transformation construct can be generated bearing donor sequences homologous to one or more of the alleles of the gene. This first transformation construct may be introduced and selection methods followed to obtain an isolated strain characterized by one or more allelic disruptions. Alternatively, a first strain may be created that is engineered to express a selectable marker from an insertion into a first allele, thereby inactivating the first allele. This strain may be used as the host for still further genetic engineering to knockout or knockdown the remaining allele(s) of the lipid pathway gene (e.g., using a second selectable marker to disrupt a second allele). Complementation of the endogenous gene can be achieved through engineered expression of an additional transformation construct bearing the endogenous gene whose activity was originally ablated, or through the expression of a suitable heterologous gene. The expression of the complementing gene can either be regulated constitutively or through regulatable control, thereby allowing for tuning of expression to the desired level so as to permit growth or create an auxotrophic condition at will. In an embodiment, a population of the fatty acid auxotroph cells are used to screen or select for complementing genes; e.g., by transformation with particular gene candidates for exogenous fatty acid synthesis enzymes, or a nucleic acid library believed to contain such candidates.

Knockout of all alleles of the desired gene and complementation of the knocked-out gene need not be carried out sequentially. The disruption of an endogenous gene of interest and its complementation either by constitutive or inducible expression of a suitable complementing gene can be carried out in several ways. In one method, this can be achieved by co-transformation of suitable constructs, one disrupting the gene of interest and the second providing complementation at a suitable, alternative locus. In another method, ablation of the target gene can be effected through the direct replacement of the target gene by a suitable gene under control of an inducible promoter (“promoter hijacking”). In this way, expression of the targeted gene is now put under the control of a regulatable promoter. An additional approach is to replace the endogenous regulatory elements of a gene with an exogenous, inducible gene expression system. Under such a regime, the gene of interest can now be turned on or off depending upon the particular needs. A still further method is to create a first strain to express an exogenous gene capable of complementing the gene of interest, then to knockout out or knockdown all alleles of the gene of interest in this first strain. The approach of multiple allelic knockdown or knockout and complementation with exogenous genes may be used to alter the fatty acid profile, regiospecific profile, sn-2 profile, or the TAG profile of the engineered cell.

Where a regulatable promoter is used, the promoter can be pH-sensitive (e.g., amt03), nitrogen and pH sensitive (e.g., amt03), or nitrogen sensitive but pH-insensitive (e.g., newly discovered promoters of Example 7) or variants thereof comprising at least 60, 65, 70, 75, 80, 85, 90, 95, 96, 97, 98 or 99% sequence identity to any of the aforementioned promoters. In connection with a promoter, pH-insensitive means that the promoter is less sensitive than the amt03 promoter when environmental conditions are shifter from pH 6.8 to 5.0 (e.g., at least 5, 10, 15, or 20% less relative change in activity upon the pH-shift as compared to an equivalent cell with amt03 as the promoter).

In a specific embodiment, the recombinant cell comprises nucleic acids operable to reduce the activity of an endogenous acyl-ACP thioesterase; for example a FatA or FatB acyl-ACP thioesterase having a preference for hydrolyzing fatty acyl-ACP chains of length C18 (e.g., stearate (C18:0) or oleate (C18:1), or C8:0-C16:0 fatty acids. The activity of an endogenous acyl-ACP thioesterase may be reduced by knockout or knockdown approaches. Knockdown may be achieved, for example, through the use of one or more RNA hairpin constructs, by promoter hijacking (substitution of a lower activity or inducible promoter for the native promoter of an endogenous gene), or by a gene knockout combined with introduction of a similar or identical gene under the control of an inducible promoter. Example 9 describes the ablation of an endogenous FATA locus and the expression of sucrose inveratase and SAD from the ablated locus.

Accordingly, oleaginous cells, including those of organisms with a type II fatty acid biosynthetic pathway can have knockouts or knockdowns of acyl-ACP thioesterase-encoding or LPAAT-encoding alleles to such a degree as to eliminate or severely limit viability of the cells in the absence of fatty acid supplementation or genetic complementations. These strains can be used to select for transformants expressing acyl-ACP-thioesterase or LPAAT transgenes.

Alternately, or in addition, the strains can be used to completely transplant exogenous acyl-ACP-thioesterases to give dramatically different fatty acid profiles of cell oils produced by such cells. For example, FATA expression can be completely or nearly completely eliminated and replaced with FATB genes that produce mid-chain fatty acids. Alternately, an organism with an endogenous FatA gene having specificity for palmitic acid (C16) relative to stearic or oleic acid (C18) can be replaced with an exogenous FatA gene having a greater relative specificity for stearic acid (C18:0) or replaced with an exogenous FatA gene having a greater relative specificity for oleic acid (C18:1). In certain specific embodiments, these transformants with double knockouts of an endogenous acyl-ACP thioesterase produce cell oils with more than 50, 60, 70, 80, or 90% caprylic, capric, lauric, myristic, or palmitic acid, or total fatty acids of chain length less than 18 carbons. Such cells may require supplementation with longer chain fatty acids such as stearic or oleic acid or switching of environmental conditions between growth permissive and restrictive states in the case of an inducible promoter regulating a FatA gene.

As discussed herein, the LPAAT enzyme catalyzes the transfer of a fatty-acyl group to the sn-2 position of a substituted acylglyceroester. Depending on the particular LPAAT, the enzyme may prefer substrates of short-chain, mid-chain or long-chain fatty-acyl groups. Certain LPAATs have broad specificity and can catalyze short-chain and mid-chain fatty-acly groups or mid-chain or long-chain fatty acyl groups.

In host cells of the invention, the host cell may have one or more endogenous LPAAT enzymes as well as having 1, 2 or more alleles encoding a particular LPAAT. The notation used herein to designate the LPAATs and their respective alleles is as follows. LPAAT1-1 designates allele 1 encoding LPAAT1; LPAAT1-2 designates allele 2 encoding LPAAT1; LPAAT2-1 designates allele 1 encoding LPAAT2; LPAAT2-2 designates allele 2 encoding LPAAT2.

In host cells of the invention, the host cell may have one or more endogenous thioesterase enzymes as well as having 1, 2 or more alleles encoding a particular thioesteras. The notation used herein to designate the thioesterases and their respective alleles is as follows. FATA-1 designates allele 1 encoding FATA; FATA-2 designates allele 2 encoding FATA; FATB-1 designates allele 1 encoding FATB; FATB-2 designates allele 2 encoding FATB.

Alternately, or in addition, the strains can be used to completely transplant exogenous LPATT to give dramatically different SN-2 profiles of cell oils produced by such cells. For example, LPAAT expression can be completely or nearly completely eliminated and replaced with LPAAT genes that catalyze the transfer of fatty-acyl groups to the SN-2 position. Alternately, an organism with an endogenous LPAAT gene having specificity for long-chain fatty-acyl groups can be replaced with an exogenous LPAAT gene having a greater relative specificity for mid-chains or replaced with an exogenous LPAAT gene having a greater relative specificity for short-chain fatty-acyl groups.

In an embodiment the oleaginous cells are cultured (e.g., in a bioreactor). The cells are fully auxotrophic or partially auxotrophic (i.e., lethality or synthetic sickness) with respect to one or more types of fatty acid. The cells are cultured with supplementation of the fatty acid(s) so as to increase the cell number, then allowing the cells to accumulate oil (e.g. to at least 40% by dry cell weight). Alternatively, the cells comprise a regulatable fatty acid synthesis gene that can be switched in activity based on environmental conditions and the environmental conditions during a first, cell division, phase favor production of the fatty acid and the environmental conditions during a second, oil accumulation, phase disfavor production of the fatty acid. In the case of an inducible gene, the regulation of the inducible gene can be mediated, without limitation, via environmental pH (for example, by using the AMTS promoter as described in the Examples).

As a result of applying either of these supplementation or regulation methods, a cell oil may be obtained from the cell that has low amounts of one or more fatty acids essential for optimal cell propagation. Specific examples of oils that can be obtained include those low in stearic, linoleic and/or linolenic acids.

These cells and methods are illustrated in connection with low polyunsaturated oils in the section immediately below.

Likewise, fatty acid auxotrophs can be made in other fatty acid synthesis genes including those encoding a SAD, FAD, KASIII, KASI, KASII, KCS, FAE, LPCAT. PDCT. DAG-CPT, GPAT, LPAAT, DGAT or AGPAT or PAP. These auxotrophs can also be used to select for complement genes or to eliminate native expression of these genes in favor of desired exogenous genes in order to alter the fatty acid profile, regiospecific profile, or TAG profile of cell oils produced by oleaginous cells.

Accordingly, in an embodiment of the invention, there is a method for producing an oil/fat. The method comprises cultivating a recombinant oleaginous cell in a growth phase under a first set of conditions that is permissive to cell division so as to increase the number of cells due to the presence of a fatty acid, cultivating the cell in an oil production phase under a second set of conditions that is restrictive to cell division but permissive to production of an oil that is depleted in the fatty acid, and extracting the oil from the cell, wherein the cell has a mutation or exogenous nucleic acids operable to suppress the activity of a fatty acid synthesis enzyme, the enzyme optionally being a stearoyl-ACP desaturase, delta 12 fatty acid desaturase, or a ketoacyl-ACP synthase, FAD, KASIII, KASI, KASII, KCS, FAE, LPCAT. PDCT. DAG-CPT, GPAT, LPAAT, DGAT or AGPAT or PAP. The oil produced by the cell can be depleted in the fatty acid by at least 50, 60, 70, 80, or 90%. The cell can be cultivated heterotrophically. The cell can be a microalgal cell cultivated heterotrophically or autotrophically and may produce at least 40, 50, 60, 70, 80, or 90% oil by dry cell weight.

### IV. Cell Oils with Less than 3% Saturated Fats

In an embodiment of the present invention, the cell oil produced by the cell has less than 3% total saturated fatty acids. The cell oil can be a liquid or solid at room temperature, or a blend of liquid and solid oils, including the regiospecific or stereospecific oils, or oils with high mono-unsaturated fatty acid content, described infra.

For example, the OSI (oxidative stability index) test may be run at temperatures between 110° C. and 140° C. The oil is produced by cultivating cells (e.g., any of the plastidic microbial cells mentioned above or elsewhere herein) that are genetically engineered to reduce the activity of one or more fatty acid desaturase. For example, the cells may be genetically engineered to reduce the activity of one or more fatty acyl Δ12 desaturase(s) responsible for converting oleic acid (18:1) into linoleic acid (18:2) and/or one or more fatty acyl Δ15 desaturase(s) responsible for converting linoleic acid (18:2) into linolenic acid (18:3). Various methods may be used to inhibit the desaturase including knockout or mutation of one or more alleles of the gene encoding the desaturase in the coding or regulatory regions, inhibition of RNA transcription, or translation of the enzyme, including RNAi, siRNA, miRNA, dsRNA, antisense, and hairpin RNA techniques. Other techniques known in the art can also be used including introducing an exogenous gene that produces an inhibitory protein or other substance that is specific for the desaturase. In specific examples, a knockout of one fatty acyl 412 desaturase allele is combined with RNA-level inhibition of a second allele. Example 9 describes an oil will less than 3% total saturated fatty acids produced by an oleaginous microalgal cell in which the FAD gene was knocked out.

In another specific embodiment there is an oil that is combined with antioxidants such as PANA and ascorbyl palmitate. Triglyceride oils and the combination of these antioxidants may have general applicability including in producing stable biodegradable lubricants (e.g., jet engine lubricants). The oxidative stability of oils can be determined by well-known techniques including the Rancimat method using the AOCS Cd 12b-92 standard test at a defined temperature. For example, the OSI (oxidative stability index) can be determined at a range of temperatures, preferably between 110° C. and 140° C.

Antioxidants suitable for use with the oils of the present invention include alpha, delta, and gamma tocopherol (vitamin E), tocotrienol, ascorbic acid (vitamin C), glutathione, lipoic acid, uric acid, β-carotene, lycopene, lutein, retinol (vitamin A), ubiquinol (coenzyme Q), melatonin, resveratrol, flavonoids, rosemary extract, propyl gallate (PG), tertiary butylhydroquinone (TBHQ), butylated hydroxyanisole (BHA), and butylated hydroxytoluene (BHT), N,N′-di-2-butyl-1,4-phenylenediamine, 2,6-di-tert-butyl-4-methylphenol, 2,4-dimethyl-6-tert-butylphenol, 2,4-dimethyl-6-tert-butylphenol, 2,4-dimethyl-6-tert-butylphenol, 2,6-di-tert-butyl-4-methylphenol, 2,6-di-tert-butylphenol, and phenyl-alpha-naphthylamine (PANA).

In addition to the desaturase modifications, in a related embodiment other genetic modifications may be made to further tailor the properties of the oil, as described throughout, including introduction or substitution of acyl-ACP thioesterases having altered chain length specificity and/or overexpression of an endogenous or exogenous gene encoding a KAS, SAD, LPAAT, DGAT, KASIII, KASI, KASII, KCS, FAE, LPCAT. PDCT. DAG-CPT, GPAT, LPAAT, DGAT or AGPAT or PAP gene. For example, a strain that produces elevated oleic levels may also produce low levels of polyunsaturates. Such genetic modifications can include increasing the activity of stearoyl-ACP desaturase (SAD) by introducing an exogenous SAD gene, increasing elongase activity by introducing an exogenous KASII gene, and/or knocking down or knocking out a FATA gene. See Example 9.

In a specific embodiment, a high oleic cell oil with low polyunsaturates may be produced. For example, the oil may have a fatty acid profile with greater than 60, 70, 80, 90, or 95% oleic acid and less than 5, 4, 3, 2, or 1% polyunsaturates. In related embodiments, a cell oil is produced by a cell having recombinant nucleic acids operable to decrease fatty acid 412 desaturase activity and optionally fatty acid 415 desaturase so as to produce an oil having less than or equal to 3% polyunsaturated fatty acids with greater than 60% oleic acid, less than 2% polyunsaturated fatty acids and greater than 70% oleic acid, less than 1% polyunsaturated fatty acids and greater than 80% oleic acid, or less than 0.5% polyunsaturated fatty acids and greater than 90% oleic acid. It has been found that one way to increase oleic acid is to use recombinant nucleic acids operable to decrease expression of a FATA acyl-ACP thioesterase and optionally overexpress a KAS II gene; such a cell can produce an oil with greater than or equal to 75% oleic acid. Alternately, overexpression of KASII can be used without the FATA knockout or knockdown. Oleic acid levels can be further increased by reduction of delta 12 fatty acid desaturase activity using the methods above, thereby decreasing the amount of oleic acid the is converted into the unsaturates linoleic acid and linolenic acid. Thus, the oil produced can have a fatty acid profile with at least 75% oleic and at most 3%, 2%, 1%, or 0.5% linoleic acid. In a related example, the oil has between 80 to 95% oleic acid and about 0.001 to 2% linoleic acid, 0.01 to 2% linoleic acid, or 0.1 to 2% linoleic acid. In another related embodiment, an oil is produced by cultivating an oleaginous cell (e.g., a microalga) so that the microbe produces a cell oil with less than 10% palmitic acid, greater than 85% oleic acid, 1% or less polyunsaturated fatty acids, and less than 7% saturated fatty acids. Such an oil is produced in a microalga with FAD and FATA knockouts plus expression of an exogenous KASII gene. Such oils will have a low freezing point, with excellent stability and are useful in foods, for frying, fuels, or in chemical applications. Further, these oils may exhibit a reduced propensity to change color over time.

### V. Cells with Exogenous Acyltransferases

In various embodiments of the present invention, one or more genes encoding an acyltransferase (an enzyme responsible for the condensation of a fatty acid with glycerol or a glycerol derivative to form an acylglyceride) can be introduced into an oleaginous cell (e.g., a plastidic microalgal cell) so as to alter the fatty acid composition of a cell oil produced by the cell. The genes may encode one or more of a glycerol-3-phosphate acyltransferase (GPAT), lysophosphatidic acid acyltransferase (LPAAT), also known as 1-acylglycerol-3-phosphate acyltransferase (AGPAT), phosphatidic acid phosphatase (PAP), or diacylglycerol acyltransferase (DGAT) that transfers an acyl group to the sn-3 position of DAG, thereby producing a TAG.

Recombinant nucleic acids may be integrated into a plasmid or chromosome of the cell. Alternately, the gene encodes an enzyme of a lipid pathway that generates TAG precursor molecules through fatty acyl-CoA-independent routes separate from that above. Acyl-ACPs may be substrates for plastidial GPAT and LPAAT enzymes and/or mitochondrial GPAT and LPAAT enzymes. Among further enzymes capable of incorporating acyl groups (e.g., from membrane phospholipids) to produce TAGs is phospholipid diacylglycerol acyltransferase (PDAT). Still further acyltransferases, including lysophosphosphatidylcholine acyltransferase (LPCAT), lysophosphosphatidylserine acyltransferase (LPSAT), lysophosphosphatidylethanolamine acyltransferase (LPEAT), and lysophosphosphatidylinositol acyltransferase (LPIAT), are involved in phospholipid synthesis and remodeling that may impact triglyceride composition.

The exogenous gene can encode an acyltransferase enzyme having preferential specificity for transferring an acyl substrate comprising a specific number of carbon atoms and/or a specific degree of saturation is introduced into a oleaginous cell so as to produce an oil enriched in a given regiospecific triglyceride. For example, the coconut (Cocos nucifera) lysophosphatidic acid acyltransferase has been demonstrated to prefer C12:0-CoA substrates over other acyl-CoA substrates (Knutzon et al., Plant Physiology, Vol. 120, 1999, pp. 739-746), whereas the 1-acyl-sn-3-glycerol-3-phosphate acyltransferase of maturing safflower seeds shows preference for linoleoyl-CoA and oleoyl-CoA substrates over other acyl-CoA substrates, including stearoyl-CoA (Ichihara et al., European Journal of Biochemistry, Vol. 167, 1989, pp. 339-347). Furthermore, acyltransferase proteins may demonstrate preferential specificity for one or more short-chain, medium-chain, or long-chain acyl-CoA or acyl-ACP substrates, but the preference may only be encountered where a particular, e.g. medium-chain, acyl group is present in the sn-1 or sn-3 position of the lysophosphatidic acid donor substrate. As a result of the exogenous gene, a TAG oil can be produced by the cell in which a particular fatty acid is found at the sn-2 position in greater than 20, 30, 40, 50, 60, 70, 90, or 90% of the TAG molecules.

In some embodiments of the invention, the cell makes an oil rich in saturated-unsaturated-saturated (sat-unsat-sat) TAGs. Sat-unsat-sat TAGS include 1,3-dihexadecanoyl-2-(9Z-octadecenoyl)-glycerol (referred to as 1-palmitoyl-2-oleyl-glycero-3-palmitoyl), 1,3-dioctadecanoyl-2-(9Z-octadecenoyl)-glycerol (referred to as 1-stearoyl-2-oleyl-glycero-3-stearoyl), and 1-hexadecanoyl-2-(9Z-octadecenoyl)-3-octadecanoy-glycerol (referred to as 1-palmitoyl-2-oleyl-glycero-3-stearoyl). These molecules are more commonly referred to as POP, SOS, and POS, respectively, where ‘P’ represents palmitic acid, ‘S’ represents stearic acid, and ‘0’ represents oleic acid. Further examples of saturated-unsaturated-saturated TAGs include MOM, LOL, MOL, COC and COL, where ‘M’ represents myristic acid, ‘L’ represents lauric acid, and ‘C’ represents capric acid (C8:0). Trisaturates, triglycerides with three saturated fatty acyl groups, are commonly sought for use in food applications for their greater rate of crystallization than other types of triglycerides. Examples of trisaturates include PPM, PPP, LLL, SSS, CCC, PPS, PPL, PPM, LLP, and LLS. In addition, the regiospecific distribution of fatty acids in a TAG is an important determinant of the metabolic fate of dietary fat during digestion and absorption.

In some embodiments, the expression of the acyltransferase, e.g., LPAAT, decreases the C18:1 content of the TAG and/or increases the C18:2, C18:3, C20:1, or C22:1 content of the TAG. Example 10 discloses the expression of LPAAT in microalgae that show significant decrease of C18:1 and significant increase in C18:2, C18:3, C20:1, or C22:1. The amount of decrease in C18:1 present in the cell oil may be decreased by lower than 10%, lower than 15%, lower than 20%, lower than 25%, lower than 30%, lower than 35%, lower than 50%, lower than 55%, lower than 60%, lower than 65%, lower than 70%, lower than 75%, lower than 80%, lower than 85%, lower than 90%, or lower than 95% than in the cell oil produced by the microorganism without the recombinant nucleic acids.

In some embodiments, the expression of the acyltransferase, e.g., LPAAT, increases the C18:2, C18:3, C20:1, or C22:1 content of the TAG. The amount of increase in C18:2, C18:3, C20:1, or C22:1 present in the cell oil may be increased by greater than 10%, greater than 15%, greater than 20%, greater than 25%, greater than 30%, greater than 35%, greater than 50%, greater than 55%, greater than 60%, greater than 65%, greater than 70%, greater than 75%, greater than 80%, greater than 85%, greater than 90%, greater than 100%, greater than 100-500%, or greater than 500% than in the cell oil produced by the microorganism without the recombinant nucleic acids.

According to certain embodiments of the present invention, oleaginous cells are transformed with recombinant nucleic acids so as to produce cell oils that comprise an elevated amount of a specified regiospecific triglyceride, for example 1-acyl-2-oleyl-glycero-3-acyl, or 1-acyl-2-lauric-glycero-3-acyl where oleic or lauric acid respectively is at the sn-2 position, as a result of introduced recombinant nucleic acids. Alternately, caprylic, capric, myristic, or palmitic acid may be at the sn-2 position. The amount of the specified regiospecific triglyceride present in the cell oil may be increased by greater than 5%, greater than 10%, greater than 15%, greater than 20%, greater than 25%, greater than 30%, greater than 35%, greater than 40%, greater than 50%, greater than 60%, greater than 70%, greater than 80%, greater than 90%, greater than 100-500%, or greater than 500% than in the cell oil produced by the microorganism without the recombinant nucleic acids. As a result, the sn-2 profile of the cell triglyceride may have greater than 10, 20, 30, 40, 50, 60, 70, 80, or 90% of the particular fatty acid.

The identity of the acyl chains located at the distinct stereospecific or regiospecific positions in a glycerolipid can be evaluated through one or more analytical methods known in the art (see Luddy et al., J. Am. Oil Chem. Soc., 41, 693-696 (1964), Brockerhoff, J. Lipid Res., 6, 10-15 (1965), Angers and Aryl, J. Am. Oil Chem. Soc., Vol. 76:4, (1999), Buchgraber et al., Eur. J. Lipid Sci. Technol., 106, 621-648 (2004)), or in accordance with Example 1 given below.

The positional distribution of fatty acids in a triglyceride molecule can be influenced by the substrate specificity of acyltransferases and by the concentration and type of available acyl moieties substrate pool. Nonlimiting examples of enzymes suitable for altering the regiospecificity of a triglyceride produced in a recombinant microorganism are listed in Tables 4-7. One of skill in the art may identify additional suitable proteins.

TABLE 4
Glycerol-3-phosphate acyltransferases and GenBank accession numbers.

| | | |
|---|---|---|
| glycerol-3-phosphate acyltransferase | Arabidopsis | BAA00575 |
| | thaliana | |
| glycerol-3-phosphate acyltransferase | Chlamydomonas | EDP02129 |
| | reinhardtii | |
| glycerol-3-phosphate acyltransferase | Chlamydomonas | Q886Q7 |
| | reinhardtii | |
| acyl-(acyl-carrier-protein): | Cucurbita moschata | BAB39688 |
| glycerol-3-phosphate acyltransferase | | |
| glycerol-3-phosphate acyltransferase | Elaeis guineensis | AAF64066 |
| glycerol-3-phosphate acyltransferase | Garcina | ABS86942 |
| | mangostana | |
| glycerol-3-phosphate acyltransferase | Gossypium hirsutum | ADK23938 |
| glycerol-3-phosphate acyltransferase | Jatropha curcas | ADV77219 |
| plastid glycerol-3-phosphate | Jatropha curcas | ACR61638 |
| acyltransferase | | |
| plastidial glycerol-phosphate | Ricinus communis | EEF43526 |
| acyltransferase | | |
| glycerol-3-phosphate acyltransferase | Vica faba | AAD05164 |
| glycerol-3-phosphate acyltransferase | Zea mays | ACG45812 |

Lysophosphatidic acid acyltransferases suitable for use with the microbes and methods of the invention include, without limitation, those listed in Table 5.

TABLE 5
Lysophosphatidic acid acyltransferases and GenBank accession numbers.

| | | |
|---|---|---|
| 1-acyl-sn-glycerol-3-phosphate acyltransferase | Arabidopsis thaliana | AEE85783 |
| 1-acyl-sn-glycerol-3-phosphate acyltransferase | Brassica juncea | ABQ42862 |
| 1-acyl-sn-glycerol-3-phosphate acyltransferase | Brassica juncea | ABM92334 |
| 1-acyl-sn-glycerol-3-phosphate acyltransferase | Brassica napus | CAB09138 |
| lysophosphatidic acid acyltransferase | Chlamydomonas | EDP02300 |
| | reinhardtii | |
| lysophosphatidic acid acyltransferase | Limnanthes alba | AAC49185 |
| 1-acyl-sn-glycerol-3-phosphate acyltransferase | Limnanthes douglasii | CAA88620 |
| (putative) | | |
| acyl-CoA:sn-1-acylglycerol-3-phosphate | Limnanthes douglasii | ABD62751 |
| acyltransferase | | |
| 1-acylglycerol-3-phosphate O-acyltransferase | Limnanthes douglasii | CAA58239 |
| 1-acyl-sn-glycerol-3-phosphate acyltransferase | Ricinus communis | EEF39377 |
| lysophosphatidic acid acyltransferase | Limnanthes douglasii | Q42870 |
| lysophosphatidic acid acyltransferase | Limnanthes alba | Q42868 |

Diacylglycerol acyltransferases suitable for use with the microbes and methods of the invention include, without limitation, those listed in Table 6.

TABLE 6
Diacylglycerol acyltransferases and GenBank accession numbers.

| | | |
|---|---|---|
| diacylglycerol acyltransferase | Arabidopsis | CAB45373 |
| | thaliana | |
| diacylglycerol acyltransferase | Brassica juncea | AAY40784 |
| putative diacylglycerol acyltransferase | Elaeis guineensis | AEQ94187 |
| putative diacylglycerol acyltransferase | Elaeis guineensis | AEQ94186 |
| acyl CoA:diacylglycerol acyltransferase | Glycine max | AAT73629 |
| diacylglycerol acyltransferase | Helianthus annus | ABX61081 |
| acyl-CoA:diacylglycerol | Olea europaea | AAS01606 |
| acyltransferase 1 | | |
| diacylglycerol acyltransferase | Ricinus communis | AAR11479 |

Phospholipid diacylglycerol acyltransferases suitable for use with the microbes and methods of the invention include, without limitation, those listed in Table 7.

TABLE 7
Phospholipid diacylglycerol acyltransferases and GenBank accession
numbers.

| | | |
|---|---|---|
| phospholipid:diacylglycerol | Arabidopsis | AED91921 |
| acyltransferase | thaliana | |
| Putative | Elaeis guineensis | AEQ94116 |
| phospholipid:diacylglycerol | | |
| acyltransferase | | |
| phospholipid:diacylglycerol | Glycine max | XP_003541296 |
| acyltransferase 1-like | | |
| phospholipid:diacylglycerol | Jatropha curcas | AEZ56255 |
| acyltransferase | | |
| phospholipid:diacylglycerol | Ricinus | ADK92410 |
| acyltransferase | communis | |
| phospholipid:diacylglycerol | Ricinus | AEW99982 |
| acyltransferase | communis | |

In an embodiment of the invention, known or novel LPAAT genes are transformed into the oleaginous cells so as to alter the fatty acid profile of triglycerides produced by those cells, by altering the sn-2 profile of the triglycerides or by increasing the C18:3, C20:1, or C22:1 content of the triglycerides or by decreasing the C18:1 content of the triglycerides. For example, by virtue of expressing an exogenous active LPAAT in an oleaginous cell, the percent of unsaturated fatty acid at the sn-2 position is increased by 10, 20, 30, 40, 50, 60, 70, 80, 90% or more. For example, a cell may produce triglycerides with 30% unsaturates (which may be primarily 18:1 and 18:2 and 18:3 fatty acids) at the sn-2 position. In another embodiment, the expression of the active LPPAT results in decreased production of C18:1 by 10%, 20%, 30%, 40%, 50%, 60%, 70%, 80%, 90% or 95%. In another embodiment, the expression of the active LPPAT results in increase production of C18:2, C18:3, C20:1, or C22:1 either individually or together by 10%, 20%, 30%, 40%, 50%, 60%, 70%, 80%, 90%, 100%, 150%, 200%, 250%, 300%, 350%, 400%, 450%, 500%, or more than 500%. Alternately, an exogenous LPAAT can be used to increase mid-chain fatty acids including saturated mid-chains such as C8:0, C10:0, C12:0, C14:0 or C16:0 moieties at the sn-2 position. As a result, mid-chain levels in the overall fatty acid profile may be increased. The choice of LPAAT gene is important in that different LPAATs can cause a shift in the sn-2 and fatty acid profiles toward different acyl group chain-lengths or saturation levels.

Specific embodiments of the invention are a nucleic acid construct, a cell comprising the nucleic acid construct, a method of cultivating the cell to produce a triglyceride, and the triglyceride oil produced where the nucleic acid construct has a promoter operably linked to a novel LPAAT coding sequence. The coding sequence can have an initiation codon upstream and a termination codon downstream followed by a 3 UTR sequence. In a specific embodiment, the LPAAT gene has LPAAT activity and a coding sequence have at least 75, 80, 85, 90, 95, 96, 97, 98, or 99% sequence identity to any of the cDNAs of SEQ ID NOs: 29 to 34 or a functional fragment thereof including equivalent sequences by virtue of degeneracy of the genetic code. Introns can be inserted into the sequence as well. In addition to microalgae and other oleaginous cells, plants expressing the novel LPAAT as transgenes are expressly included in the embodiments and can be produced using known genetic engineering techniques.

### VI. Cells with Exogenous Elongases or Elongase Complex Enzymes

In various embodiments of the present invention, one or more genes encoding elongases or components of the fatty acyl-CoA elongation complex can be introduced into an oleaginous cell (e.g., a plastidic microalgal cell) so as to alter the fatty acid composition of the cell or of a cell oil produced by the cell. The genes may encode a beta-ketoacyl-CoA synthase (also referred to as Elongase, 3-ketoacyl synthase, beta-ketoacyl synthase or KCS), a ketoacyl-CoA reductase, a hydroxyacyl-CoA dehydratase, enoyl-CoA reductase, or elongase. The enzymes encoded by these genes are active in the elongation of acyl-coA molecules liberated by acyl-ACP thioesterases. Recombinant nucleic acids may be integrated into a plasmid or chromosome of the cell. In a specific embodiment, the cell is of Chlorophyta, including heterotrophic cells such as those of the genus Prototheca.

Beta-Ketoacyl-CoA synthase and elongase enzymes suitable for use with the microbes and methods of the invention include, without limitation, those listed in Table 8 and in the sequence listing.

TABLE 8
Beta-Ketoacyl-CoA synthases and elongases listed with GenBank
accession numbers.

Trypanosoma brucei elongase 3 (GenBank Accession No. AAX70673), Marchanita
polymorpha (GenBank Accession No. AAP74370), Trypanosoma cruzi fatty acid elongase,
putative (GenBank Accession No. EFZ33366), Nannochloropsis oculata fatty acid elongase
(GenBank Accession No. ACV21066.1), Leishmania donovani fatty acid elongase, putative
(GenBank Accession No. CBZ32733.1), Glycine max 3-ketoacyl-CoA synthase 11-like
(GenBank Accession No. XP_003524525.1), Medicago truncatula beta-ketoacyl-CoA
synthase (GenBank Accession No. XP_003609222), Zea mays fatty acid elongase (GenBank
Accession No. ACG36525), Gossypium hirsutum beta-ketoacyl-CoA synthase (GenBank
Accession No. ABV60087), Helianthus annuus beta-ketoacyl-CoA synthase (GenBank
Accession No. ACC60973.1), Saccharomyces cerevisiae ELO1 (GenBank Accession No.
P39540), Simmondsia chinensis beta-ketoacyl-CoA synthase (GenBank Accession No.
AAC49186), Tropaeolum majus putative fatty acid elongase (GenBank Accession No.
AAL99199, Brassica napus fatty acid elongase (GenBank Accession No. AAA96054)

In an embodiment of the invention, an exogenous gene encoding a beta-ketoacyl-CoA synthase or elongase enzyme having preferential specificity for elongating an acyl substrate comprising a specific number of carbon atoms and/or a specific degree of acyl chain saturation is introduced into a oleaginous cell so as to produce a cell or an oil enriched in fatty acids of specified chain length and/or saturation. Examples 10 and 15 describe engineering of Prototheca strains in which exogenous fatty acid elongases with preferences for extending long-chain fatty acyl-CoAs have been overexpressed to increase the concentration of C18:2, C18:3, C20:1, and/or C22:1.

In specific embodiments, the oleaginous cell produces an oil comprising greater than 0.5, 1, 2, 5, 10, 20, 30, 40, 50, 60 70, or 80% linoleic, linolenic, erucic and/or eicosenoic acid. Alternately, the cell produces an oil comprising 0.5-5, 5-10, 10-15, 15-20, 20-30, 30-40, 40-50, 50-60, 60-70, 70-80, 80-90, or 90-99% linoleic, linolenic, erucic or eicosenoic acid. The cell may comprise recombinant acids described above in connection with high-oleic oils with a further introduction of an exogenous beta-ketoacyl-CoA synthase that is active in elongating oleoyl-CoA. As a result of the expression of the exogenous beta-ketoacyl-CoA synthase, the natural production of linolenic, erucic or eicosenoic acid by the cell can be increased by more than 2, 3, 4, 5, 10, 20, 30, 40, 50, 70, 100, 130, 170, 200, 250, 300, 350, Or 400 fold. The high erucic and/or eicosenoic oil can also be a high stability oil; e.g., one comprising less than 5, 4, 3, 2, or 1% polyunsaturates and/or having the OSI values described in Section IV or this application and accompanying Examples. In a specific embodiment, the cell is a microalgal cell, optionally cultivated heterotrophically. As in the other embodiments, the oil/fat can be produced by genetic engineering of a plastidic cell, including heterotrophic microalgae of the phylum Chlorophyta, the class Trebouxiophytae, the order Chlorellales, or the family Chlorellacae. Preferably, the cell is oleaginous and capable of accumulating at least 40% oil by dry cell weight. The cell can be an obligate heterotroph, such as a species of Prototheca, including Prototheca moriformis or Prototheca zopfii.

In specific embodiments, an oleaginous microbial cell, optionally an oleaginous microalgal cell, optionally of the phylum Chlorophyta, the class Trebouxiophytae, the order Chlorellales, or the family Chlorellacae expresses an enzyme having 80, 85, 90, 95, 96, 97, 98, or 99% amino acid sequence identity to an enzyme of Table 8.

### VII. Regiospecific and Stereospecific Oils/Fats

In an embodiment, a recombinant cell produces a cell fat or oil having a given regiospecific makeup. As a result, the cell can produce triglyceride fats having a tendency to form crystals of a given polymorphic form; e.g., when heated to above melting temperature and then cooled to below melting temperature of the fat. For example, the fat may tend to form crystal polymorphs of the β or β′ form (e.g., as determined by X-ray diffraction analysis), either with or without tempering. The fats may be ordered fats. In specific embodiments, the fat may directly from either β or β′ crystals upon cooling; alternatively, the fat can proceed through a β form to a β′ form. Such fats can be used as structuring, laminating or coating fats for food applications. The cell fats can be incorporated into candy, dark or white chocolate, chocolate flavored confections, ice cream, margarines or other spreads, cream fillings, pastries, or other food products. Optionally, the fats can be semi-solid (at room temperature) yet free of artificially produced trans-fatty acids. Such fats can also be useful in skin care and other consumer or industrial products.

As in the other embodiments, the fat can be produced by genetic engineering of a plastidic cell, including heterotrophic eukaryotic microalgae of the phylum Chlorophyta, the class Trebouxiophytae, the order Chlorellales, or the family Chlorellacae. Preferably, the cell is oleaginous and capable of accumulating at least 40% oil by dry cell weight. The cell can be an obligate heterotroph, such as a species of Prototheca, including Prototheca moriformis or Prototheca zopfii. The fats can also be produced in autotrophic algae or plants. Optionally, the cell is capable of using sucrose to produce oil and a recombinant invertase gene may be introduced to allow metabolism of sucrose, as described in PCT Publications WO2008/151149, WO2010/06032, WO2011/150410, WO2011/150411, and international patent application PCT/US12/23696. The invertase may be codon optimized and integrated into a chromosome of the cell, as may all of the genes mentioned here. It has been found that cultivated recombinant microalgae can produce hardstock fats at temperatures below the melting point of the hardstock fat. For example, Prototheca moriformis can be altered to heterotrophically produce triglyceride oil with greater than 50% stearic acid at temperatures in the range of 15 to 30° C., wherein the oil freezes when held at 30° C.

In an embodiment, the cell fat has at least 30, 40, 50, 60, 70, 80, or 90% fat of the general structure [saturated fatty acid (sn-1)-unsaturated fatty acid (sn-2)-saturated fatty acid (sn-3)]. This is denoted below as Sat-Unsat-Sat fat. In a specific embodiment, the saturated fatty acid in this structure is preferably stearate or palmitate and the unsaturated fatty acid is preferably oleate. As a result, the fat can form primarily β or β′ polymorphic crystals, or a mixture of these, and have corresponding physical properties, including those desirable for use in foods or personal care products. For example, the fat can melt at mouth temperature for a food product or skin temperature for a cream, lotion or other personal care product (e.g., a melting temperature of 30 to 40, or 32 to 35° C.). Optionally, the fats can have a 2 L or 3 L lamellar structure (e.g., as determined by X-ray diffraction analysis). Optionally, the fat can form this polymorphic form without tempering.

In a specific related embodiment, a cell fat triglyceride has a high concentration of SOS (i.e. triglyceride with stearate at the terminal sn-1 and sn-3 positions, with oleate at the sn-2 position of the glycerol backbone). For example, the fat can have triglycerides comprising at least 50, 60, 70, 80 or 90% SOS. In an embodiment, the fat has triglyceride of at least 80% SOS. Optionally, at least 50, 60, 70, 80 or 90% of the sn-2 linked fatty acids are unsaturated fatty acids. In a specific embodiment, at least 95% of the sn-2 linked fatty acids are unsaturated fatty acids. In addition, the SSS (tri-stearate) level can be less than 20, 10 or 5% and/or the C20:0 fatty acid (arachidic acid) level may be less than 6%, and optionally greater than 1% (e.g., from 1 to 5%). For example, in a specific embodiment, a cell fat produced by a recombinant cell has at least 70% SOS triglyceride with at least 80% sn-2 unsaturated fatty acyl moieties. In another specific embodiment, a cell fat produced by a recombinant cell has TAGs with at least 80% SOS triglyceride and with at least 95% sn-2 unsaturated fatty acyl moieties. In yet another specific embodiment, a cell fat produced by a recombinant cell has TAGs with at least 80% SOS, with at least 95% sn-2 unsaturated fatty acyl moieties, and between 1 to 6% C20 fatty acids.

In yet another specific embodiment, the sum of the percent stearate and palmitate in the fatty acid profile of the cell fat is twice the percentage of oleate, ±10, 20, 30 or 40% [e.g., (% P+% S)/% O=2.0±20%]. Optionally, the sn-2 profile of this fat is at least 40%, and preferably at least 50, 60, 70, or 80% oleate (at the sn-2 position). Also optionally, this fat may be at least 40, 50, 60, 70, 80, or 90% SOS. Optionally, the fat comprises between 1 to 6% C20 fatty acids.

In any of these embodiments, the high SatUnsatSat fat may tend to form β′ polymorphic crystals. Unlike previously available plant fats like cocoa butter, the SatUnsatSat fat produced by the cell may form β′ polymorphic crystals without tempering. In an embodiment, the polymorph forms upon heating to above melting temperature and cooling to less that the melting temperature for 3, 2, 1, or 0.5 hours. In a related embodiment, the polymorph forms upon heating to above 60° C. and cooling to 10° C. for 3, 2, 1, or 0.5 hours.

In various embodiments the fat forms polymorphs of the β form, β′ form, or both, when heated above melting temperature and the cooled to below melting temperature, and optionally proceeding to at least 50% of polymorphic equilibrium within 5, 4, 3, 2, 1, 0.5 hours or less when heated to above melting temperature and then cooled at 10° C. The fat may form β′ crystals at a rate faster than that of cocoa butter.

Optionally, any of these fats can have less than 2 mole % diacylglycerol, or less than 2 mole % mono and diacylglycerols, in sum.

In an embodiment, the fat may have a melting temperature of between 30-60° C., 30-40° C., 32 to 37° C., 40 to 60° C. or 45 to 55° C. In another embodiment, the fat can have a solid fat content (SFC) of 40 to 50%, 15 to 25%, or less than 15% at 20° C. and/or have an SFC of less than 15% at 35° C.

The cell used to make the fat may include recombinant nucleic acids operable to modify the saturate to unsaturate ratio of the fatty acids in the cell triglyceride in order to favor the formation of SatUnsatSat fat. For example, a knock-out or knock-down of stearoyl-ACP desaturase (SAD) gene can be used to favor the formation of stearate over oleate or expression of an exogenous mid-chain-preferring acyl-ACP thioesterase gene can increase the levels mid-chain saturates. Alternately a gene encoding a SAD enzyme can be overexpressed to increase unsaturates.

In a specific embodiment, the cell has recombinant nucleic acids operable to elevate the level of stearate in the cell. As a result, the concentration of SOS may be increased. Another genetic modification to increase stearate levels includes increasing a ketoacyl ACP synthase (KAS) activity in the cell so as to increase the rate of stearate production. Methods of increasing the level of sterate in the cell are described in WO2012/1106560, WO2013/158938, and PCT/US2014/059161.

The cell oils invention can be distinguished from conventional vegetable or animal triacylglycerol sources in that the sterol profile will be indicative of the host organism as distinguishable from the conventional source. Conventional sources of oil include soy, corn, sunflower, safflower, palm, palm kernel, coconut, cottonseed, canola, rape, peanut, olive, flax, tallow, lard, cocoa, shea, mango, sal, illipe, kokum, and allanblackia. See section XIII of this disclosure for a discussion of microalgal sterols.

### VIII. Cells Expressing a Recombinant Nucleic Acid Encoding LPCAT, PDCT, DAG-PCT and/or FAE and Oils Enriched in C18:2, C18:3, C20:1 and C22:1

Lysophosphatidylcholine acyltransferase (LPCAT) enzymes play a central role in acyl editing of phosphatidylcholine (PC). LPCAT enzymes work in both forward and reversible reaction modes. In the forward mode, they are responsible for the channeling of fatty acids into PC (at both available sn positions). In the reverse reaction mode, LPCAT enzymes transfer of fatty acid out of PC into the acyl CoA pool. The liberated fatty acid can then be incorporated into the formation of a TAG or further desaturated or elongated. In the case of a liberated oleic acid, it can be incorporated into the formation of a TAG or can be further processed to linoleic acid, linolenic acid or further elongated to C20:1, C22:1 or more highly desaturated fatty acids which then can be incorporated to form a TAG.

Phosphotidylcholine diacylglycerol cholinephosphotransferase (PDCT) and diacylglycerol cholinephosphotransferas (DAG-CPT) catalyze the removal of linoleic acid or linolenic acid from PC. The liberated fatty acids can then can be incorporated into the formation of a TAG or further elongated to C20:1 or C22:1 or more highly desaturated fatty acids which then can be incorporated to form a TAG.

In various embodiments of the present invention, one or more nucleic acids encoding LPCAT, PDCT, DAG-CPT and/or FAE can be introduced into an oleaginous cell (e.g., a plastidic microalgal cell) so as to alter the fatty acid composition of the cell or of a cell oil produced by the cell. Recombinant nucleic acids may be integrated into a plasmid or chromosome of the cell. In a specific embodiment, the cell is of Chlorophyta, including heterotrophic cells such as those of the genus Prototheca.

In some embodiments, the expression of the LPCAT, PDCT, DAG-CPT, and/or FAE decreases the C18:1 content of the TAG and/or increases the C18:2, C18:3, C20:1, or C22:1 content of the TAG. Examples 11, 12 and 16 disclose the expression of LPCAT in microalgae that show significant decrease of C18:1 and significant increase in C18:2, C18:3, C20:1, or C22:1. Examples 13 and 14 disclose the expression of PDCT in microalgae that show significant decrease of C18:1 and significant increase in C18:2, C18:3, C20:1, or C22:1. Example 15 discloses the expression of DAG-CPT in microalgae that show significant decrease of C18:1 and significant increase in C18:2, C18:3, C20:1, or C22:1. The amount of decrease in C18:1 present in the cell oil may be decreased by lower than 10%, lower than 15%, lower than 20%, lower than 25%, lower than 30%, lower than 35%, lower than 50%, lower than 55%, lower than 60%, lower than 65%, lower than 70%, lower than 75%, lower than 80%, lower than 85%, lower than 90%, or lower than 95% than in the cell oil produced by the microorganism without the recombinant nucleic acids.

In some embodiments, the expression of the LPCAT, PDCT, DAG-CPT, and/or FAE increases the C18:2, C18:3, C20:1, or C22:1 content of the TAG. The amount of increase in C18:2, C18:3, C20:1, or C22:1 present in the cell oil may be increased by greater than 10%, greater than 15%, greater than 20%, greater than 25%, greater than 30%, greater than 35%, greater than 50%, greater than 55%, greater than 60%, greater than 65%, greater than 70%, greater than 75%, greater than 80%, greater than 85%, greater than 90%, greater than 100%, greater than 100-500%, or greater than 500% than in the cell oil produced by the microorganism without the recombinant nucleic acids.

### IX. Cells with an Ablation of an Endogenous Gene and a Recombinant Nucleic Acid Encoding LPCAT, PDCT, DAG-Pct and/or FAE and Oils Enriched in C18:2, C18:3, C20:1 and C22:1

One embodiment of the invention is a recombinant cell in which one, two or all the alleles of an endogenous gene is ablated (knocked-out) and one or more recombinant nucleic acids encoding LPCAT, PDCT, DAG-PCT, AND/OR FAE is expressed. Optionally, the gene that is ablated is a lipid biosynthetic pathway gene. Alternately, the amount or activity of the gene products of the alleles is knocked down, for example by inhibitory RNA technologies including RNAi, siRNA, miRNA, dsRNA, antisense, and hairpin RNA techniques. so as to require supplementation with fatty acids. When one allele of the lipid pathway gene is knocked out, a corresponding decrease in the enzymatic activity is observed. When all alleles of the lipid pathway gene are knocked out or sufficiently inhibited an auxotroph is created. As discussed herein, constructs can be generated bearing donor sequences homologous to one or more of the alleles of the gene. This first transformation construct may be introduced and selection methods followed to obtain an isolated strain characterized by one or more allelic disruptions. Alternatively, a first strain may be created that is engineered to express a selectable marker from an insertion into a first allele, thereby inactivating the first allele. This strain may be used as the host for still further genetic engineering to knockout or knockdown the remaining allele(s) of the lipid pathway gene (e.g., using a second selectable marker to disrupt a second allele).

In some embodiments, an allele that is ablated is also locus for insertion of the nucleic acids encoding encoding LPCAT, PDCT, DAG-PCT and/or FAE. In one embodiment the allele that is knocked-out is a gene that encodes an LPAAT. In Example 10, one allele of LPAAT1, designated as LPAAT1-1 was ablated and served as the locus for insertion of a nucleic acid encoding LPAAT. Also in Example 10, the 6S site served as the locus for insertion of a nucleic acid encoding FAE. In Examples 11, one allele of LPAAT1, designated as LPAAT1-1 was ablated and served as the locus for insertion of a nucleic acid encoding LPCAT. Example 11 also discloses ablation of LPAAT1-1 which served as the locus for insertion of a nucleic acid encoding FAE. In Example 13, LPAAT1-1 (allele 1), or LPAAT1-2 (allele 2) served as the locus for insertion of a nucleic acid encoding PDCT. Example 13 also discloses insertion of FAE into the 6S site. In Example 14, LPAAT1-1 was the locus for insertion of PDCT. In Example 15, LPAAT1-1 or LPAAT2-2 was the locus for insertion of DAG-PCT. Example 15 also discloses insertion of FAE into the 6S site. In Example 16, LPAAT1-1 was the locus for insertion of LPCAT. Example 16 also discloses insertion of FAE into the 6S site.

In some embodiments, the ablation of a lipid biosynthetic pathway gene, optionally LPAAT, and expression of the LPCAT, PDCT, DAG-CPT, and/or FAE decreases the C18:1 content of the TAG and/or increases the C18:2, C18:3, C20:1, or C22:1 content of the TAG. The amount of decrease in C18:1 present in the cell oil may be decreased by lower than 10%, lower than 15%, lower than 20%, lower than 25%, lower than 30%, lower than 35%, lower than 50%, lower than 55%, lower than 60%, lower than 65%, lower than 70%, lower than 75%, lower than 80%, lower than 85%, lower than 90%, or lower than 95% than in the cell oil produced by the microorganism without the recombinant nucleic acids.

In some embodiments, the ablation of a lipid biosynthetic pathway gene, optionally LPAAT, the expression of the LPCAT, PDCT, DAG-CPT, and/or FAE increases the C18:2, C18:3, C20:1, or C22:1 content of the TAG. The amount of increase in C18:2, C18:3, C20:1, or C22:1 present in the cell oil may be increased by greater than 10%, greater than 15%, greater than 20%, greater than 25%, greater than 30%, greater than 35%, greater than 50%, greater than 55%, greater than 60%, greater than 65%, greater than 70%, greater than 75%, greater than 80%, greater than 85%, greater than 90%, greater than 100%, greater than 100-500%, or greater than 500% than in the cell oil produced by the microorganism without the recombinant nucleic acids.

### X. Low Saturate Oil

In an embodiment, a cell oil is produced from a recombinant cell. The oil produced has a fatty acid profile that has less that 4%, 3%, 2%, or 1% (area %), saturated fatty acids. In a specific embodiment, the oil has 0.1 to 5%, 0.1 to 4%, or 0.1 to 3.5% saturated fatty acids. Certain of such oils can be used to produce a food with negligible amounts of saturated fatty acids. Optionally, these oils can have fatty acid profiles comprising at least 90% oleic acid or at least 90% oleic acid with at least 3% polyunsaturated fatty acids. In an embodiment, a cell oil produced by a recombinant cell comprises at least 90% oleic acid, at least 3% of the sum of linoleic and linolenic acid, or at least 2% of the sum of linoleic and linolenic acid, and has less than 4%, or less than 3.5% saturated fatty acids. In a related embodiment, a cell oil produced by a recombinant cell comprises at least 90% oleic acid, at least 3% of the sum of linoleic and linolenic acid and has less than 4%, or less than 3.5% saturated fatty acids, the majority of the saturated fatty acids being comprised of chain length 10 to 16. In a related embodiment, a cell oil produced by a recombinant cell comprises at least 90% oleic acid, at least 2% or 3% of the sum of linoleic and linolenic acid, has less than 3.5% saturated fatty acids and comprises at least 0.5%, at least 1%, or at least 2% palmitic acid. These oils may be produced by recombinant oleaginous cells including but not limited to those described here and in U.S. patent application Ser. No. 13/365,253. For example, overexpression of a KASII enzyme in a cell with a highly active SAD can produce a high oleic oil with less than or equal to 3.75%, 3.6% or 3.5% saturates. Optionally, an oleate-specific acyl-ACP thioesterase is also overexpressed and/or an endogenous thioesterase having a propensity to hydrolyze acyl chains of less than C18 knocked out or suppressed. The oleate-specific acyl-ACP thioesterase may be a transgene with low activity toward ACP-palmitate and ACP-stearate so that the ratio of oleic acid relative to the sum of palmitic acid and stearic acid in the fatty acid profile of the oil produced is greater than 3, 5, 7, or 10. Alternately, or in addition, a FATA gene may be knocked out or knocked down. A FATA gene may be knocked out or knocked down and an exogenous KASII overexpressed. Another optional modification is to increase KASI and/or KASIII activity, which can further suppress the formation of shorter chain saturates. Optionally, one or more acyltransferases (e.g., an LPAAT) having specificity for transferring unsaturated fatty acyl moieties to a substituted glycerol is also overexpressed and/or an endogenous acyltransferase is knocked out or attenuated. An additional optional modification is to increase the activity of KCS enzymes having specificity for elongating unsaturated fatty acids and/or an endogenous KCS having specificity for elongating saturated fatty acids is knocked out or attenuated. Optionally, oleate is increased at the expense of linoleate production by knockout or knockdown of a delta 12 fatty acid desaturase. Optionally, the exogenous genes used can be plant genes; e.g., obtained from cDNA derived from mRNA found in oil seeds. Example 9 discloses a cell oil with less than 3.5% saturated fatty acids.

In addition to the above genetic modifications, the low saturate oil can be a high-stability oil by virtue of low amounts of polyunsaturated fatty acids. Methods and characterizations of high-stability, low-polyunsaturated oils are described herein, including method to reduce the activity of endogenous 412 fatty acid desaturase. In a specific embodiment, an oil is produced by a oleaginous microbial cell having a type II fatty acid synthetic pathway and has no more than 3.5% saturated fatty acids and also has no more than 3% polyunsaturated fatty acids. In another specific embodiment, the oil has no more than 3% saturated fatty acids and also has no more than 2% polyunsaturated fatty acids. In another specific embodiment, the oil has no more than 3% saturated fatty acids and also has no more than 1% polyunsaturated fatty acids. In another specific embodiment, a eukaryotic microalgal cell comprises an exogenous gene that desaturates palmitic acid to palmitoleic acid in operable linkage with regulatory elements operable in the microalgal cell. The cell further comprises a knockout or knockdown of a FAD gene. Due to the genetic modifications, the cell produces a cell oil having a fatty acid profile in which the ratio of palmitoleic acid (C16:1) to palmitic acid (C16:0) is greater than 0.1, with no more than 3% polyunsaturated fatty acids. Optionally, palmitoleic acid comprises 0.5% or more of the profile. Optionally, the cell oil comprises less than 3.5% saturated fatty acids.

The low saturate and low saturate/high stability oil can be blended with less expensive oils to reach a targeted saturated fatty acid level at less expense. For example, an oil with 1% saturated fat can be blended with an oil having 7% saturated fat (e.g. high-oleic sunflower oil) to give an oil having 3.5% or less saturated fat.

Oils produced according to embodiments of the present invention can be used in the transportation fuel, oleochemical, and/or food and cosmetic industries, among other applications. For example, transesterification of lipids can yield long-chain fatty acid esters useful as biodiesel. Other enzymatic and chemical processes can be tailored to yield fatty acids, aldehydes, alcohols, alkanes, and alkenes. In some applications, renewable diesel, jet fuel, or other hydrocarbon compounds are produced. The present disclosure also provides methods of cultivating microalgae for increased productivity and increased lipid yield, and/or for more cost-effective production of the compositions described herein. The methods described here allow for the production of oils from plastidic cell cultures at large scale; e.g., 1000, 10,000, 100,000 liters or more.

In an embodiment, an oil extracted from the cell has 3.5%, 3%, 2.5%, or 2% saturated fat or less and is incorporated into a food product. The finished food product has 3.5, 3, 2.5, or 2% saturated fat or less. For example, oils recovered from such recombinant microalgae can be used for frying oils or as an ingredient in a prepared food that is low in saturated fats. The oils can be used neat or blended with other oils so that the food has less than 0.5 g of saturated fat per serving, thus allowing a label stating zero saturated fat (per US regulation). In a specific embodiment, the oil has a fatty acid profile with at least 90% oleic acid, less than 3% saturated fat, and more oleic acid than linoleic acid.

As with the other oils disclosed in this patent application, the low-saturate oils described in this section, including those with increased levels palmitoleic acid, can have a microalgal sterol profile as described in Section XIII of this application. For example, via expression of an exogenous PAD gene, an oil can be produced with a fatty acid profile characterized by a ratio of palmitoleic acid to palmitic acid of at least 0.1 and/or palmitoleic acid levels of 0.5% or more, as determined by FAME GC/FID analysis and a sterol profile characterized by an excess of ergosterol over β-sitosterol and/or the presence of 22, 23-dihydrobrassicasterol, poriferasterol or clionasterol.

### XI. Minor Oil Components

The oils produced according to the above methods in some cases are made using a microalgal host cell. As described above, the microalga can be, without limitation, fall in the classification of Chlorophyta, Trebouxiophyceae, Chlorellales, Chlorellaceae, or Chlorophyceae. It has been found that microalgae of Trebouxiophyceae can be distinguished from vegetable oils based on their sterol profiles. Oil produced by Chlorella protothecoides was found to produce sterols that appeared to be brassicasterol, ergosterol, campesterol, stigmasterol, and β-sitosterol, when detected by GC-MS. However, it is believed that all sterols produced by Chlorella have C24β stereochemistry. Thus, it is believed that the molecules detected as campesterol, stigmasterol, and β-sitosterol, are actually 22,23-dihydrobrassicasterol, poriferasterol and clionasterol, respectively. Thus, the oils produced by the microalgae described above can be distinguished from plant oils by the presence of sterols with C24β stereochemistry and the absence of C24α stereochemistry in the sterols present. For example, the oils produced may contain 22, 23-dihydrobrassicasterol while lacking campesterol; contain clionasterol, while lacking in β-sitosterol, and/or contain poriferasterol while lacking stigmasterol. Alternately, or in addition, the oils may contain significant amounts of Δ⁷-poriferasterol.

In one embodiment, the oils provided herein are not vegetable oils. Vegetable oils are oils extracted from plants and plant seeds. Vegetable oils can be distinguished from the non-plant oils provided herein on the basis of their oil content. A variety of methods for analyzing the oil content can be employed to determine the source of the oil or whether adulteration of an oil provided herein with an oil of a different (e.g. plant) origin has occurred. The determination can be made on the basis of one or a combination of the analytical methods. These tests include but are not limited to analysis of one or more of free fatty acids, fatty acid profile, total triacylglycerol content, diacylglycerol content, peroxide values, spectroscopic properties (e.g. UV absorption), sterol profile, sterol degradation products, antioxidants (e.g. tocopherols), pigments (e.g. chlorophyll), d13C values and sensory analysis (e.g. taste, odor, and mouth feel). Many such tests have been standardized for commercial oils such as the Codex Alimentarius standards for edible fats and oils.

Sterol profile analysis is a particularly well-known method for determining the biological source of organic matter. Campesterol, b-sitosterol, and stigmasterol are common plant sterols, with β-sitosterol being a principle plant sterol. For example, β-sitosterol was found to be in greatest abundance in an analysis of certain seed oils, approximately 64% in corn, 29% in rapeseed, 64% in sunflower, 74% in cottonseed, 26% in soybean, and 79% in olive oil (Gul et al. J. Cell and Molecular Biology 5:71-79, 2006).

Oil isolated from Prototheca moriformis strain UTEX1435 were separately clarified (CL), refined and bleached (RB), or refined, bleached and deodorized (RBD) and were tested for sterol content according to the procedure described in JAOCS vol. 60, no. 8, August 1983. Results of the analysis are shown below (units in mg/100 g) in Table 9.

TABLE 9
Sterol profiles of oils from UTEX 1435.

| | | | | | Refined, |
|---|---|---|---|---|---|
| | | | | Refined & | bleached, & |
| | Sterol | Crude | Clarified | bleached | deodorized |
| 1 | Ergosterol | 384 | 398 | 293 | 302 |
| | | (56%) | (55%) | (50%) | (50%) |
| 2 | 5,22-cholestadien- | 14.6 | 18.8 | 14 | 15.2 |
| | 24-methyl-3-ol | (2.1%) | (2.6%) | (2.4%) | (2.5%) |
| | (Brassicasterol) | | | | |
| 3 | 24-methylcholest- | 10.7 | 11.9 | 10.9 | 10.8 |
| | 5-en-3-ol | (1.6%) | (1.6%) | (1.8%) | (1.8%) |
| | (Campesterol or | | | | |
| | 22,23-dihydro- | | | | |
| | brassicasterol) | | | | |
| 4 | 5,22-cholestadien- | 57.7 | 59.2 | 46.8 | 49.9 |
| | 24-ethyl-3-ol | (8.4%) | (8.2%) | (7.9%) | (8.3%) |
| | (Stigmasterol | | | | |
| | or poriferasterol) | | | | |
| 5 | 24-ethylcholest-5- | 9.64 | 9.92 | 9.26 | 10.2 |
| | en-3-ol (β-Sitosterol | (1.4%) | (1.4%) | (1.6%) | (1.7%) |
| | or clionasterol) | | | | |
| 6 | Other sterols | 209 | 221 | 216 | 213 |
| | Total sterols | 685.64 | 718.82 | 589.96 | 601.1 |

These results show three striking features. First, ergosterol was found to be the most abundant of all the sterols, accounting for about 50% or more of the total sterols. The amount of ergosterol is greater than that of campesterol, β-sitosterol, and stigmasterol combined. Ergosterol is steroid commonly found in fungus and not commonly found in plants, and its presence particularly in significant amounts serves as a useful marker for non-plant oils. Secondly, the oil was found to contain brassicasterol. With the exception of rapeseed oil, brassicasterol is not commonly found in plant based oils. Thirdly, less than 2% β-sitosterol was found to be present. β-sitosterol is a prominent plant sterol not commonly found in microalgae, and its presence particularly in significant amounts serves as a useful marker for oils of plant origin. In summary, Prototheca moriformis strain UTEX1435 has been found to contain both significant amounts of ergosterol and only trace amounts of β-sitosterol as a percentage of total sterol content. Accordingly, the ratio of ergosterol:β-sitosterol or in combination with the presence of brassicasterol can be used to distinguish this oil from plant oils.

In some embodiments, the oil content of an oil provided herein contains, as a percentage of total sterols, less than 20%, 15%, 10%, 5%, 4%, 3%, 2%, or 1% β-sitosterol. In other embodiments the oil is free from β-sitosterol. For any of the oils or cell-oils disclosed in this application, the oil can have the sterol profile of any column of Table 9, above, with a sterol-by-sterol variation of 30%, 20%, 10% or less.

In some embodiments, the oil is free from one or more of β-sitosterol, campesterol, or stigmasterol. In some embodiments the oil is free from β-sitosterol, campesterol, and stigmasterol. In some embodiments the oil is free from campesterol. In some embodiments the oil is free from stigmasterol.

In some embodiments, the oil content of an oil provided herein comprises, as a percentage of total sterols, less than 20%, 15%, 10%, 5%, 4%, 3%, 2%, or 1% 24-ethylcholest-5-en-3-ol. In some embodiments, the 24-ethylcholest-5-en-3-ol is clionasterol. In some embodiments, the oil content of an oil provided herein comprises, as a percentage of total sterols, at least 1%, 2%, 3%, 4%, 5%, 6%, 7%, 8%, 9%, or 10% clionasterol.

In some embodiments, the oil content of an oil provided herein contains, as a percentage of total sterols, less than 20%, 15%, 10%, 5%, 4%, 3%, 2%, or 1% 24-methylcholest-5-en-3-ol. In some embodiments, the 24-methylcholest-5-en-3-ol is 22, 23-dihydrobrassicasterol. In some embodiments, the oil content of an oil provided herein comprises, as a percentage of total sterols, at least 1%, 2%, 3%, 4%, 5%, 6%, 7%, 8%, 9%, or 10% 22,23-dihydrobrassicasterol.

In some embodiments, the oil content of an oil provided herein contains, as a percentage of total sterols, less than 20%, 15%, 10%, 5%, 4%, 3%, 2%, or 1% 5,22-cholestadien-24-ethyl-3-ol. In some embodiments, the 5, 22-cholestadien-24-ethyl-3-ol is poriferasterol. In some embodiments, the oil content of an oil provided herein comprises, as a percentage of total sterols, at least 1%, 2%, 3%, 4%, 5%, 6%, 7%, 8%, 9%, or 10% poriferasterol.

In some embodiments, the oil content of an oil provided herein contains ergosterol or brassicasterol or a combination of the two. In some embodiments, the oil content contains, as a percentage of total sterols, at least 5%, 10%, 20%, 25%, 35%, 40%, 45%, 50%, 55%, 60%, or 65% ergosterol. In some embodiments, the oil content contains, as a percentage of total sterols, at least 25% ergosterol. In some embodiments, the oil content contains, as a percentage of total sterols, at least 40% ergosterol. In some embodiments, the oil content contains, as a percentage of total sterols, at least 5%, 10%, 20%, 25%, 35%, 40%, 45%, 50%, 55%, 60%, or 65% of a combination of ergosterol and brassicasterol.

In some embodiments, the oil content contains, as a percentage of total sterols, at least 1%, 2%, 3%, 4% or 5% brassicasterol. In some embodiments, the oil content contains, as a percentage of total sterols less than 10%, 9%, 8%, 7%, 6%, or 5% brassicasterol.

In some embodiments the ratio of ergosterol to brassicasterol is at least 5:1, 10:1, 15:1, or 20:1.

In some embodiments, the oil content contains, as a percentage of total sterols, at least 5%, 10%, 20%, 25%, 35%, 40%, 45%, 50%, 55%, 60%, or 65% ergosterol and less than 20%, 15%, 10%, 5%, 4%, 3%, 2%, or 1% β-sitosterol. In some embodiments, the oil content contains, as a percentage of total sterols, at least 25% ergosterol and less than 5% β-sitosterol. In some embodiments, the oil content further comprises brassicasterol.

Sterols contain from 27 to 29 carbon atoms (C27 to C29) and are found in all eukaryotes. Animals exclusively make C27 sterols as they lack the ability to further modify the C27 sterols to produce C28 and C29 sterols. Plants however are able to synthesize C28 and C29 sterols, and C28/C29 plant sterols are often referred to as phytosterols. The sterol profile of a given plant is high in C29 sterols, and the primary sterols in plants are typically the C29 sterols b-sitosterol and stigmasterol. In contrast, the sterol profile of non-plant organisms contain greater percentages of C27 and C28 sterols. For example the sterols in fungi and in many microalgae are principally C28 sterols. The sterol profile and particularly the striking predominance of C29 sterols over C28 sterols in plants has been exploited for determining the proportion of plant and marine matter in soil samples (Huang, Wen-Yen, Meinschein W. G., “Sterols as ecological indicators”; Geochimica et Cosmochimia Acta. Vol 43. pp 739-745).

In some embodiments the primary sterols in the microalgal oils provided herein are sterols other than b-sitosterol and stigmasterol. In some embodiments of the microalgal oils, C29 sterols make up less than 50%, 40%, 30%, 20%, 10%, or 5% by weight of the total sterol content.

In some embodiments the microalgal oils provided herein contain C28 sterols in excess of C29 sterols. In some embodiments of the microalgal oils, C28 sterols make up greater than 50%, 60%, 70%, 80%, 90%, or 95% by weight of the total sterol content. In some embodiments the C28 sterol is ergosterol. In some embodiments the C28 sterol is brassicasterol.

### XII. Fuels and Chemicals

The oils discussed above alone or in combination are useful in the production of foods, fuels and chemicals (including plastics, foams, films, etc.). The oils, triglycerides, fatty acids from the oils may be subjected to C—H activation, hydroamino methylation, methoxy-carbonation, ozonolysis, enzymatic transformations, epoxidation, methylation, dimerization, thiolation, metathesis, hydro-alkylation, lactonization, or other chemical processes.

The oils can be converted to alkanes (e.g., renewable diesel) or esters (e.g., methyl or ethyl esters for biodisesel produced by transesterification). The alkanes or esters may be used as fuel, as solvents or lubricants, or as a chemical feedstock. Methods for production of renewable diesel and biodiesel are well established in the art. See, for example, WO2011/150411.

In a specific embodiment of the present invention, a high-oleic or high-oleic-high stability oil described above is esterified. For example, the oils can be transesterified with methanol to an oil that is rich in methyl oleate. Such formulations have been found to compare favorably with methyl oleate from soybean oil.

In another specific example, the oil is converted to C36 diacids or products of C36 diacids. Fatty acids produced from the oil can be polymerized to give a composition rich in C36 dimer acids. In a specific example, high-oleic oil is split to give a high-oleic fatty acid material which is polymerized to give a composition rich in C36-dimer acids. Optionally, the oil is high oleic high stability oil (e.g., greater than 60% oleic acid with less than 3% polyunsaturates, greater than 70% oleic acid with less than 2% polyunsaturates, or greater than 80% oleic acid with less than 1% polyunsaturates). It is believed that using a high oleic, high stability, starting material will give lower amounts of cyclic products, which may be desirable in some cases. After hydrolyzing the oil, one obtains a high concentration of oleic acid. In the process of making dimer acids, a high oleic acid stream will convert to a “cleaner” C36 dimer acid and not produce trimers acids (C54) and other more complex cyclic by-products which are obtained due to presence of C18:2 and C18:3 acids. For example, the oil can be hydrolyzed to fatty acids and the fatty acids purified and dimerized at 250° C. in the presence of montmorillonite clay. See SRI Natural Fatty Acid, March 2009. A product rich in C36 dimers of oleic acid is recovered.

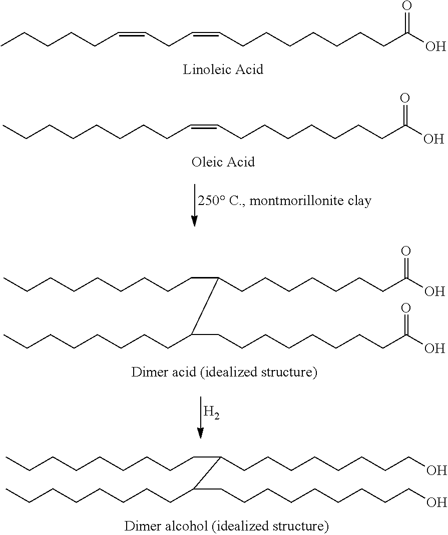 [SMILES: CCCCC/C=C\C/C=C\CCCCCCCC(=O)O ; CCCCCCCC/C=C\CCCCCCCC(=O)O ; CCCCCCCCCC(CCCCCCCC(=O)O)C(CCCCCCCC)CCCCCCCCC(=O)O ; "H2" ; CCCCCCCCCC(CCCCCCCCO)C(CCCCCCCC)CCCCCCCCCO]

Further, the C36 dimer acids can be esterified and hydrogenated to give diols. The diols can be polymerized by catalytic dehydration. Polymers can also be produced by transesterification of dimerdiols with dimethyl carbonate.

For the production of fuel in accordance with the methods of the invention lipids produced by cells of the invention are harvested, or otherwise collected, by any convenient means. Lipids can be isolated by whole cell extraction. The cells are first disrupted, and then intracellular and cell membrane/cell wall-associated lipids as well as extracellular hydrocarbons can be separated from the cell mass, such as by use of centrifugation. Intracellular lipids produced in oleaginous cells are, in some embodiments, extracted after lysing the cells. Once extracted, the lipids are further refined to produce oils, fuels, or oleochemicals.

Various methods are available for separating lipids from cellular lysates. For example, lipids and lipid derivatives such as fatty aldehydes, fatty alcohols, and hydrocarbons such as alkanes can be extracted with a hydrophobic solvent such as hexane (see Frenz et al. 1989, Enzyme Microb. Technol., 11:717). Lipids and lipid derivatives can also be extracted using liquefaction (see for example Sawayama et al. 1999, Biomass and Bioenergy 17:33-39 and Inoue et al. 1993, Biomass Bioenergy 6(4):269-274); oil liquefaction (see for example Minowa et al. 1995, Fuel 74(12):1735-1738); and supercritical CO₂ extraction (see for example Mendes et al. 2003, Inorganica Chimica Acta 356:328-334). Miao and Wu describe a protocol of the recovery of microalgal lipid from a culture of Chlorella protothecoides in which the cells were harvested by centrifugation, washed with distilled water and dried by freeze drying. The resulting cell powder was pulverized in a mortar and then extracted with n-hexane. Miao and Wu, Biosource Technology (2006) 97:841-846.

Lipids and lipid derivatives can be recovered by extraction with an organic solvent. In some cases, the preferred organic solvent is hexane. Typically, the organic solvent is added directly to the lysate without prior separation of the lysate components. In one embodiment, the lysate generated by one or more of the methods described above is contacted with an organic solvent for a period of time sufficient to allow the lipid and/or hydrocarbon components to form a solution with the organic solvent. In some cases, the solution can then be further refined to recover specific desired lipid or hydrocarbon components. Hexane extraction methods are well known in the art.

Lipids produced by cells in vivo, or enzymatically modified in vitro, as described herein can be optionally further processed by conventional means. The processing can include “cracking” to reduce the size, and thus increase the hydrogen:carbon ratio, of hydrocarbon molecules. Catalytic and thermal cracking methods are routinely used in hydrocarbon and triglyceride oil processing. Catalytic methods involve the use of a catalyst, such as a solid acid catalyst. The catalyst can be silica-alumina or a zeolite, which result in the heterolytic, or asymmetric, breakage of a carbon-carbon bond to result in a carbocation and a hydride anion. These reactive intermediates then undergo either rearrangement or hydride transfer with another hydrocarbon. The reactions can thus regenerate the intermediates to result in a self-propagating chain mechanism. Hydrocarbons can also be processed to reduce, optionally to zero, the number of carbon-carbon double, or triple, bonds therein. Hydrocarbons can also be processed to remove or eliminate a ring or cyclic structure therein. Hydrocarbons can also be processed to increase the hydrogen:carbon ratio. This can include the addition of hydrogen (“hydrogenation”) and/or the “cracking” of hydrocarbons into smaller hydrocarbons.

Thermal methods involve the use of elevated temperature and pressure to reduce hydrocarbon size. An elevated temperature of about 800° C. and pressure of about 700 kPa can be used. These conditions generate “light,” a term that is sometimes used to refer to hydrogen-rich hydrocarbon molecules (as distinguished from photon flux), while also generating, by condensation, heavier hydrocarbon molecules which are relatively depleted of hydrogen. The methodology provides homolytic, or symmetrical, breakage and produces alkenes, which may be optionally enzymatically saturated as described above.

Catalytic and thermal methods are standard in plants for hydrocarbon processing and oil refining. Thus hydrocarbons produced by cells as described herein can be collected and processed or refined via conventional means. See Hillen et al. (Biotechnology and Bioengineering, Vol. XXIV:193-205 (1982)) for a report on hydrocracking of microalgae-produced hydrocarbons. In alternative embodiments, the fraction is treated with another catalyst, such as an organic compound, heat, and/or an inorganic compound. For processing of lipids into biodiesel, a transesterification process is used as described below in this Section.

Hydrocarbons produced via methods of the present invention are useful in a variety of industrial applications. For example, the production of linear alkylbenzene sulfonate (LAS), an anionic surfactant used in nearly all types of detergents and cleaning preparations, utilizes hydrocarbons generally comprising a chain of 10-14 carbon atoms. See, for example, U.S. Pat. Nos. 6,946,430; 5,506,201; 6,692,730; 6,268,517; 6,020,509; 6,140,302; 5,080,848; and 5,567,359. Surfactants, such as LAS, can be used in the manufacture of personal care compositions and detergents, such as those described in U.S. Pat. Nos. 5,942,479; 6,086,903; 5,833,999; 6,468,955; and 6,407,044.

Increasing interest is directed to the use of hydrocarbon components of biological origin in fuels, such as biodiesel, renewable diesel, and jet fuel, since renewable biological starting materials that may replace starting materials derived from fossil fuels are available, and the use thereof is desirable. There is an urgent need for methods for producing hydrocarbon components from biological materials. The present invention fulfills this need by providing methods for production of biodiesel, renewable diesel, and jet fuel using the lipids generated by the methods described herein as a biological material to produce biodiesel, renewable diesel, and jet fuel.

Traditional diesel fuels are petroleum distillates rich in paraffinic hydrocarbons. They have boiling ranges as broad as 370° to 780° F., which are suitable for combustion in a compression ignition engine, such as a diesel engine vehicle. The American Society of Testing and Materials (ASTM) establishes the grade of diesel according to the boiling range, along with allowable ranges of other fuel properties, such as cetane number, cloud point, flash point, viscosity, aniline point, sulfur content, water content, ash content, copper strip corrosion, and carbon residue. Technically, any hydrocarbon distillate material derived from biomass or otherwise that meets the appropriate ASTM specification can be defined as diesel fuel (ASTM D975), jet fuel (ASTM D1655), or as biodiesel if it is a fatty acid methyl ester (ASTM D6751).

After extraction, lipid and/or hydrocarbon components recovered from the microbial biomass described herein can be subjected to chemical treatment to manufacture a fuel for use in diesel vehicles and jet engines.

Biodiesel is a liquid which varies in color—between golden and dark brown—depending on the production feedstock. It is practically immiscible with water, has a high boiling point and low vapor pressure. Biodiesel refers to a diesel-equivalent processed fuel for use in diesel-engine vehicles. Biodiesel is biodegradable and non-toxic. An additional benefit of biodiesel over conventional diesel fuel is lower engine wear. Typically, biodiesel comprises C14-C18 alkyl esters. Various processes convert biomass or a lipid produced and isolated as described herein to diesel fuels. A preferred method to produce biodiesel is by transesterification of a lipid as described herein. A preferred alkyl ester for use as biodiesel is a methyl ester or ethyl ester.

Biodiesel produced by a method described herein can be used alone or blended with conventional diesel fuel at any concentration in most modern diesel-engine vehicles. When blended with conventional diesel fuel (petroleum diesel), biodiesel may be present from about 0.1% to about 99.9%. Much of the world uses a system known as the “B” factor to state the amount of biodiesel in any fuel mix. For example, fuel containing 20% biodiesel is labeled B20. Pure biodiesel is referred to as B100.

Biodiesel can be produced by transesterification of triglycerides contained in oil-rich biomass. Thus, in another aspect of the present invention a method for producing biodiesel is provided. In a preferred embodiment, the method for producing biodiesel comprises the steps of (a) cultivating a lipid-containing microorganism using methods disclosed herein (b) lysing a lipid-containing microorganism to produce a lysate, (c) isolating lipid from the lysed microorganism, and (d) transesterifying the lipid composition, whereby biodiesel is produced. Methods for growth of a microorganism, lysing a microorganism to produce a lysate, treating the lysate in a medium comprising an organic solvent to form a heterogeneous mixture and separating the treated lysate into a lipid composition have been described above and can also be used in the method of producing biodiesel. The lipid profile of the biodiesel is usually highly similar to the lipid profile of the feedstock oil.

Lipid compositions can be subjected to transesterification to yield long-chain fatty acid esters useful as biodiesel. Preferred transesterification reactions are outlined below and include base catalyzed transesterification and transesterification using recombinant lipases. In a base-catalyzed transesterification process, the triacylglycerides are reacted with an alcohol, such as methanol or ethanol, in the presence of an alkaline catalyst, typically potassium hydroxide. This reaction forms methyl or ethyl esters and glycerin (glycerol) as a byproduct.

Transesterification has also been carried out, as discussed above, using an enzyme, such as a lipase instead of a base. Lipase-catalyzed transesterification can be carried out, for example, at a temperature between the room temperature and 80° C., and a mole ratio of the TAG to the lower alcohol of greater than 1:1, preferably about 3:1. Other examples of lipases useful for transesterification are found in, e.g., U.S. Pat. Nos. 4,798,793; 4,940,845 5,156,963; 5,342,768; 5,776,741 and WO89/01032. Such lipases include, but are not limited to, lipases produced by microorganisms of Rhizopus, Aspergillus, Candida, Mucor, Pseudomonas, Rhizomucor, Candida, and Humicola and pancreas lipase.

Subsequent processes may also be used if the biodiesel will be used in particularly cold temperatures. Such processes include winterization and fractionation. Both processes are designed to improve the cold flow and winter performance of the fuel by lowering the cloud point (the temperature at which the biodiesel starts to crystallize). There are several approaches to winterizing biodiesel. One approach is to blend the biodiesel with petroleum diesel. Another approach is to use additives that can lower the cloud point of biodiesel. Another approach is to remove saturated methyl esters indiscriminately by mixing in additives and allowing for the crystallization of saturates and then filtering out the crystals. Fractionation selectively separates methyl esters into individual components or fractions, allowing for the removal or inclusion of specific methyl esters. Fractionation methods include urea fractionation, solvent fractionation and thermal distillation.

Another valuable fuel provided by the methods of the present invention is renewable diesel, which comprises alkanes, such as C10:0, C12:0, C14:0, C16:0 and C18:0 and thus, are distinguishable from biodiesel. High quality renewable diesel conforms to the ASTM D975 standard. The lipids produced by the methods of the present invention can serve as feedstock to produce renewable diesel. Thus, in another aspect of the present invention, a method for producing renewable diesel is provided. Renewable diesel can be produced by at least three processes: hydrothermal processing (hydrotreating); hydroprocessing; and indirect liquefaction. These processes yield non-ester distillates. During these processes, triacylglycerides produced and isolated as described herein, are converted to alkanes.

In one embodiment, the method for producing renewable diesel comprises (a) cultivating a lipid-containing microorganism using methods disclosed herein (b) lysing the microorganism to produce a lysate, (c) isolating lipid from the lysed microorganism, and (d) deoxygenating and hydrotreating the lipid to produce an alkane, whereby renewable diesel is produced. Lipids suitable for manufacturing renewable diesel can be obtained via extraction from microbial biomass using an organic solvent such as hexane, or via other methods, such as those described in U.S. Pat. No. 5,928,696. Some suitable methods may include mechanical pressing and centrifuging.

In some methods, the microbial lipid is first cracked in conjunction with hydrotreating to reduce carbon chain length and saturate double bonds, respectively. The material is then isomerized, also in conjunction with hydrotreating. The naptha fraction can then be removed through distillation, followed by additional distillation to vaporize and distill components desired in the diesel fuel to meet an ASTM D975 standard while leaving components that are heavier than desired for meeting the D975 standard. Hydrotreating, hydrocracking, deoxygenation and isomerization methods of chemically modifying oils, including triglyceride oils, are well known in the art. See for example European patent applications EP1741768 (A1); EP1741767 (A1); EP1682466 (A1); EP1640437 (A1); EP1681337 (A1); EP1795576 (A1); and U.S. Pat. Nos. 7,238,277; 6,630,066; 6,596,155; 6,977,322; 7,041,866; 6,217,746; 5,885,440; 6,881,873.

In one embodiment of the method for producing renewable diesel, treating the lipid to produce an alkane is performed by hydrotreating of the lipid composition. In hydrothermal processing, typically, biomass is reacted in water at an elevated temperature and pressure to form oils and residual solids. Conversion temperatures are typically 300° to 660° F., with pressure sufficient to keep the water primarily as a liquid, 100 to 170 standard atmosphere (atm). Reaction times are on the order of 15 to 30 minutes. After the reaction is completed, the organics are separated from the water. Thereby a distillate suitable for diesel is produced.

In some methods of making renewable diesel, the first step of treating a triglyceride is hydroprocessing to saturate double bonds, followed by deoxygenation at elevated temperature in the presence of hydrogen and a catalyst. In some methods, hydrogenation and deoxygenation occur in the same reaction. In other methods deoxygenation occurs before hydrogenation. Isomerization is then optionally performed, also in the presence of hydrogen and a catalyst. Naphtha components are preferably removed through distillation. For examples, see U.S. Pat. No. 5,475,160 (hydrogenation of triglycerides); U.S. Pat. No. 5,091,116 (deoxygenation, hydrogenation and gas removal); U.S. Pat. No. 6,391,815 (hydrogenation); and U.S. Pat. No. 5,888,947 (isomerization).

One suitable method for the hydrogenation of triglycerides includes preparing an aqueous solution of copper, zinc, magnesium and lanthanum salts and another solution of alkali metal or preferably, ammonium carbonate. The two solutions may be heated to a temperature of about 20° C. to about 85° C. and metered together into a precipitation container at rates such that the pH in the precipitation container is maintained between 5.5 and 7.5 in order to form a catalyst. Additional water may be used either initially in the precipitation container or added concurrently with the salt solution and precipitation solution. The resulting precipitate may then be thoroughly washed, dried, calcined at about 300° C. and activated in hydrogen at temperatures ranging from about 100° C. to about 400° C. One or more triglycerides may then be contacted and reacted with hydrogen in the presence of the above-described catalyst in a reactor. The reactor may be a trickle bed reactor, fixed bed gas-solid reactor, packed bubble column reactor, continuously stirred tank reactor, a slurry phase reactor, or any other suitable reactor type known in the art. The process may be carried out either batchwise or in continuous fashion. Reaction temperatures are typically in the range of from about 170° C. to about 250° C. while reaction pressures are typically in the range of from about 300 psig to about 2000 psig. Moreover, the molar ratio of hydrogen to triglyceride in the process of the present invention is typically in the range of from about 20:1 to about 700:1. The process is typically carried out at a weight hourly space velocity (WHSV) in the range of from about 0.1 h⁻¹ to about 5 h⁻¹. One skilled in the art will recognize that the time period required for reaction will vary according to the temperature used, the molar ratio of hydrogen to triglyceride, and the partial pressure of hydrogen. The products produced by the such hydrogenation processes include fatty alcohols, glycerol, traces of paraffins and unreacted triglycerides. These products are typically separated by conventional means such as, for example, distillation, extraction, filtration, crystallization, and the like.

Petroleum refiners use hydroprocessing to remove impurities by treating feeds with hydrogen. Hydroprocessing conversion temperatures are typically 300° to 700° F. Pressures are typically 40 to 100 atm. The reaction times are typically on the order of 10 to 60 minutes. Solid catalysts are employed to increase certain reaction rates, improve selectivity for certain products, and optimize hydrogen consumption.

Suitable methods for the deoxygenation of an oil includes heating an oil to a temperature in the range of from about 350° F. to about 550° F. and continuously contacting the heated oil with nitrogen under at least pressure ranging from about atmospheric to above for at least about 5 minutes.

Suitable methods for isomerization include using alkali isomerization and other oil isomerization known in the art.

Hydrotreating and hydroprocessing ultimately lead to a reduction in the molecular weight of the triglyceride feed. The triglyceride molecule is reduced to four hydrocarbon molecules under hydroprocessing conditions: a propane molecule and three heavier hydrocarbon molecules, typically in the C8 to C18 range.

Thus, in one embodiment, the product of one or more chemical reaction(s) performed on lipid compositions of the invention is an alkane mixture that comprises ASTM D975 renewable diesel. Production of hydrocarbons by microorganisms is reviewed by Metzger et al. Appl Microbiol Biotechnol (2005) 66: 486-496 and A Look Back at the U.S. Department of Energy's Aquatic Species Program: Biodiesel from Algae, NREL/TP-580-24190, John Sheehan, Terri Dunahay, John Benemann and Paul Roessler (1998).

The distillation properties of a diesel fuel is described in terms of T10-T90 (temperature at 10% and 90%, respectively, volume distilled). Methods of hydrotreating, isomerization, and other covalent modification of oils disclosed herein, as well as methods of distillation and fractionation (such as cold filtration) disclosed herein, can be employed to generate renewable diesel compositions with other T10-T90 ranges, such as 20, 25, 30, 35, 40, 45, 50, 60 and 65° C. using triglyceride oils produced according to the methods disclosed herein.

Methods of hydrotreating, isomerization, and other covalent modification of oils disclosed herein, as well as methods of distillation and fractionation (such as cold filtration) disclosed herein, can be employed to generate renewable diesel compositions with other T10 values, such as T10 between 180 and 295, between 190 and 270, between 210 and 250, between 225 and 245, and at least 290.

Methods of hydrotreating, isomerization, and other covalent modification of oils disclosed herein, as well as methods of distillation and fractionation (such as cold filtration) disclosed herein can be employed to generate renewable diesel compositions with certain T90 values, such as T90 between 280 and 380, between 290 and 360, between 300 and 350, between 310 and 340, and at least 290.

Methods of hydrotreating, isomerization, and other covalent modification of oils disclosed herein, as well as methods of distillation and fractionation (such as cold filtration) disclosed herein, can be employed to generate renewable diesel compositions with other FBP values, such as FBP between 290 and 400, between 300 and 385, between 310 and 370, between 315 and 360, and at least 300.

Other oils provided by the methods and compositions of the invention can be subjected to combinations of hydrotreating, isomerization, and other covalent modification including oils with lipid profiles including (a) at least 1%-5%, preferably at least 4%, C8-C14; (b) at least 0.25%-1%, preferably at least 0.3%, C8; (c) at least 1%-5%, preferably at least 2%, C10; (d) at least 1%-5%, preferably at least 2%, C12; and (3) at least 20%-40%, preferably at least 30% C8-C14.

A traditional ultra-low sulfur diesel can be produced from any form of biomass by a two-step process. First, the biomass is converted to a syngas, a gaseous mixture rich in hydrogen and carbon monoxide. Then, the syngas is catalytically converted to liquids. Typically, the production of liquids is accomplished using Fischer-Tropsch (FT) synthesis. This technology applies to coal, natural gas, and heavy oils. Thus, in yet another preferred embodiment of the method for producing renewable diesel, treating the lipid composition to produce an alkane is performed by indirect liquefaction of the lipid composition.

The present invention also provides methods to produce jet fuel. Jet fuel is clear to straw colored. The most common fuel is an unleaded/paraffin oil-based fuel classified as Aeroplane A-1, which is produced to an internationally standardized set of specifications. Jet fuel is a mixture of a large number of different hydrocarbons, possibly as many as a thousand or more. The range of their sizes (molecular weights or carbon numbers) is restricted by the requirements for the product, for example, freezing point or smoke point. Kerosene-type Aeroplane fuel (including Jet A and Jet A-1) has a carbon number distribution between about 8 and 16 carbon numbers. Wide-cut or naphtha-type Aeroplane fuel (including Jet B) typically has a carbon number distribution between about 5 and 15 carbons.

In one embodiment of the invention, a jet fuel is produced by blending algal fuels with existing jet fuel. The lipids produced by the methods of the present invention can serve as feedstock to produce jet fuel. Thus, in another aspect of the present invention, a method for producing jet fuel is provided. Herewith two methods for producing jet fuel from the lipids produced by the methods of the present invention are provided: fluid catalytic cracking (FCC); and hydrodeoxygenation (HDO).

Fluid Catalytic Cracking (FCC) is one method which is used to produce olefins, especially propylene from heavy crude fractions. The lipids produced by the method of the present invention can be converted to olefins. The process involves flowing the lipids produced through an FCC zone and collecting a product stream comprised of olefins, which is useful as a jet fuel. The lipids produced are contacted with a cracking catalyst at cracking conditions to provide a product stream comprising olefins and hydrocarbons useful as jet fuel.

In one embodiment, the method for producing jet fuel comprises (a) cultivating a lipid-containing microorganism using methods disclosed herein, (b) lysing the lipid-containing microorganism to produce a lysate, (c) isolating lipid from the lysate, and (d) treating the lipid composition, whereby jet fuel is produced. In one embodiment of the method for producing a jet fuel, the lipid composition can be flowed through a fluid catalytic cracking zone, which, in one embodiment, may comprise contacting the lipid composition with a cracking catalyst at cracking conditions to provide a product stream comprising C2-05 olefins.

In certain embodiments of this method, it may be desirable to remove any contaminants that may be present in the lipid composition. Thus, prior to flowing the lipid composition through a fluid catalytic cracking zone, the lipid composition is pretreated. Pretreatment may involve contacting the lipid composition with an ion-exchange resin. The ion exchange resin is an acidic ion exchange resin, such as Amberlyst™-15 and can be used as a bed in a reactor through which the lipid composition is flowed, either upflow or downflow. Other pretreatments may include mild acid washes by contacting the lipid composition with an acid, such as sulfuric, acetic, nitric, or hydrochloric acid. Contacting is done with a dilute acid solution usually at ambient temperature and atmospheric pressure.

The lipid composition, optionally pretreated, is flowed to an FCC zone where the hydrocarbonaceous components are cracked to olefins. Catalytic cracking is accomplished by contacting the lipid composition in a reaction zone with a catalyst composed of finely divided particulate material. The reaction is catalytic cracking, as opposed to hydrocracking, and is carried out in the absence of added hydrogen or the consumption of hydrogen. As the cracking reaction proceeds, substantial amounts of coke are deposited on the catalyst. The catalyst is regenerated at high temperatures by burning coke from the catalyst in a regeneration zone. Coke-containing catalyst, referred to herein as “coked catalyst”, is continually transported from the reaction zone to the regeneration zone to be regenerated and replaced by essentially coke-free regenerated catalyst from the regeneration zone. Fluidization of the catalyst particles by various gaseous streams allows the transport of catalyst between the reaction zone and regeneration zone. Methods for cracking hydrocarbons, such as those of the lipid composition described herein, in a fluidized stream of catalyst, transporting catalyst between reaction and regeneration zones, and combusting coke in the regenerator are well known by those skilled in the art of FCC processes. Exemplary FCC applications and catalysts useful for cracking the lipid composition to produce C2-05 olefins are described in U.S. Pat. Nos. 6,538,169, 7,288,685, which are incorporated in their entirety by reference.

Suitable FCC catalysts generally comprise at least two components that may or may not be on the same matrix. In some embodiments, both two components may be circulated throughout the entire reaction vessel. The first component generally includes any of the well-known catalysts that are used in the art of fluidized catalytic cracking, such as an active amorphous clay-type catalyst and/or a high activity, crystalline molecular sieve. Molecular sieve catalysts may be preferred over amorphous catalysts because of their much-improved selectivity to desired products. In some preferred embodiments, zeolites may be used as the molecular sieve in the FCC processes. Preferably, the first catalyst component comprises a large pore zeolite, such as a Y-type zeolite, an active alumina material, a binder material, comprising either silica or alumina and an inert filler such as kaolin.

In one embodiment, cracking the lipid composition of the present invention, takes place in the riser section or, alternatively, the lift section, of the FCC zone. The lipid composition is introduced into the riser by a nozzle resulting in the rapid vaporization of the lipid composition. Before contacting the catalyst, the lipid composition will ordinarily have a temperature of about 149° C. to about 316° C. (300° F. to 600° F.). The catalyst is flowed from a blending vessel to the riser where it contacts the lipid composition for a time of abort 2 seconds or less.

The blended catalyst and reacted lipid composition vapors are then discharged from the top of the riser through an outlet and separated into a cracked product vapor stream including olefins and a collection of catalyst particles covered with substantial quantities of coke and generally referred to as “coked catalyst.” In an effort to minimize the contact time of the lipid composition and the catalyst which may promote further conversion of desired products to undesirable other products, any arrangement of separators such as a swirl arm arrangement can be used to remove coked catalyst from the product stream quickly. The separator, e.g. swirl arm separator, is located in an upper portion of a chamber with a stripping zone situated in the lower portion of the chamber. Catalyst separated by the swirl arm arrangement drops down into the stripping zone. The cracked product vapor stream comprising cracked hydrocarbons including light olefins and some catalyst exit the chamber via a conduit which is in communication with cyclones. The cyclones remove remaining catalyst particles from the product vapor stream to reduce particle concentrations to very low levels. The product vapor stream then exits the top of the separating vessel. Catalyst separated by the cyclones is returned to the separating vessel and then to the stripping zone. The stripping zone removes adsorbed hydrocarbons from the surface of the catalyst by counter-current contact with steam.

Low hydrocarbon partial pressure operates to favor the production of light olefins. Accordingly, the riser pressure is set at about 172 to 241 kPa (25 to 35 psia) with a hydrocarbon partial pressure of about 35 to 172 kPa (5 to 25 psia), with a preferred hydrocarbon partial pressure of about 69 to 138 kPa (10 to 20 psia). This relatively low partial pressure for hydrocarbon is achieved by using steam as a diluent to the extent that the diluent is 10 to 55 wt-% of lipid composition and preferably about 15 wt-% of lipid composition. Other diluents such as dry gas can be used to reach equivalent hydrocarbon partial pressures.

The temperature of the cracked stream at the riser outlet will be about 510° C. to 621° C. (950° F. to 1150° F.). However, riser outlet temperatures above 566° C. (1050° F.) make more dry gas and more olefins. Whereas, riser outlet temperatures below 566° C. (1050° F.) make less ethylene and propylene. Accordingly, it is preferred to run the FCC process at a preferred temperature of about 566° C. to about 630° C., preferred pressure of about 138 kPa to about 240 kPa (20 to 35 psia). Another condition for the process is the catalyst to lipid composition ratio which can vary from about 5 to about 20 and preferably from about 10 to about 15.

In one embodiment of the method for producing a jet fuel, the lipid composition is introduced into the lift section of an FCC reactor. The temperature in the lift section will be very hot and range from about 700° C. (1292° F.) to about 760° C. (1400° F.) with a catalyst to lipid composition ratio of about 100 to about 150. It is anticipated that introducing the lipid composition into the lift section will produce considerable amounts of propylene and ethylene.

In another embodiment of the method for producing a jet fuel using the lipid composition or the lipids produced as described herein, the structure of the lipid composition or the lipids is broken by a process referred to as hydrodeoxygenation (HDO). HDO means removal of oxygen by means of hydrogen, that is, oxygen is removed while breaking the structure of the material. Olefinic double bonds are hydrogenated and any sulfur and nitrogen compounds are removed. Sulfur removal is called hydrodesulphurization (HDS). Pretreatment and purity of the raw materials (lipid composition or the lipids) contribute to the service life of the catalyst.

Generally in the HDO/HDS step, hydrogen is mixed with the feed stock (lipid composition or the lipids) and then the mixture is passed through a catalyst bed as a co-current flow, either as a single phase or a two phase feed stock. After the HDO/MDS step, the product fraction is separated and passed to a separate isomerization reactor. An isomerization reactor for biological starting material is described in the literature (FI 100 248) as a co-current reactor.

The process for producing a fuel by hydrogenating a hydrocarbon feed, e.g., the lipid composition or the lipids herein, can also be performed by passing the lipid composition or the lipids as a co-current flow with hydrogen gas through a first hydrogenation zone, and thereafter the hydrocarbon effluent is further hydrogenated in a second hydrogenation zone by passing hydrogen gas to the second hydrogenation zone as a counter-current flow relative to the hydrocarbon effluent. Exemplary HDO applications and catalysts useful for cracking the lipid composition to produce C2-05 olefins are described in U.S. Pat. No. 7,232,935, which is incorporated in its entirety by reference.

Typically, in the hydrodeoxygenation step, the structure of the biological component, such as the lipid composition or lipids herein, is decomposed, oxygen, nitrogen, phosphorus and sulfur compounds, and light hydrocarbons as gas are removed, and the olefinic bonds are hydrogenated. In the second step of the process, i.e. in the so-called isomerization step, isomerization is carried out for branching the hydrocarbon chain and improving the performance of the paraffin at low temperatures.

In the first step, i.e. HDO step, of the cracking process, hydrogen gas and the lipid composition or lipids herein which are to be hydrogenated are passed to a HDO catalyst bed system either as co-current or counter-current flows, said catalyst bed system comprising one or more catalyst bed(s), preferably 1-3 catalyst beds. The HDO step is typically operated in a co-current manner. In case of a HDO catalyst bed system comprising two or more catalyst beds, one or more of the beds may be operated using the counter-current flow principle. In the HDO step, the pressure varies between 20 and 150 bar, preferably between 50 and 100 bar, and the temperature varies between 200 and 500° C., preferably in the range of 300−400° C. In the HDO step, known hydrogenation catalysts containing metals from Group VII and/or VIB of the Periodic System may be used. Preferably, the hydrogenation catalysts are supported Pd, Pt, Ni, NiMo or a CoMo catalysts, the support being alumina and/or silica. Typically, NiMo/Al₂O₃ and CoMo/Al₂O₃ catalysts are used.

Prior to the HDO step, the lipid composition or lipids herein may optionally be treated by prehydrogenation under milder conditions thus avoiding side reactions of the double bonds. Such prehydrogenation is carried out in the presence of a prehydrogenation catalyst at temperatures of 50−400° C. and at hydrogen pressures of 1-200 bar, preferably at a temperature between 150 and 250° C. and at a hydrogen pressure between 10 and 100 bar. The catalyst may contain metals from Group VIII and/or VIB of the Periodic System. Preferably, the prehydrogenation catalyst is a supported Pd, Pt, Ni, NiMo or a CoMo catalyst, the support being alumina and/or silica.

A gaseous stream from the HDO step containing hydrogen is cooled and then carbon monoxide, carbon dioxide, nitrogen, phosphorus and sulfur compounds, gaseous light hydrocarbons and other impurities are removed therefrom. After compressing, the purified hydrogen or recycled hydrogen is returned back to the first catalyst bed and/or between the catalyst beds to make up for the withdrawn gas stream. Water is removed from the condensed liquid. The liquid is passed to the first catalyst bed or between the catalyst beds.

After the HDO step, the product is subjected to an isomerization step. It is substantial for the process that the impurities are removed as completely as possible before the hydrocarbons are contacted with the isomerization catalyst. The isomerization step comprises an optional stripping step, wherein the reaction product from the HDO step may be purified by stripping with water vapor or a suitable gas such as light hydrocarbon, nitrogen or hydrogen. The optional stripping step is carried out in counter-current manner in a unit upstream of the isomerization catalyst, wherein the gas and liquid are contacted with each other, or before the actual isomerization reactor in a separate stripping unit utilizing counter-current principle.

After the stripping step the hydrogen gas and the hydrogenated lipid composition or lipids herein, and optionally an n-paraffin mixture, are passed to a reactive isomerization unit comprising one or several catalyst bed(s). The catalyst beds of the isomerization step may operate either in co-current or counter-current manner.

It is important for the process that the counter-current flow principle is applied in the isomerization step. In the isomerization step this is done by carrying out either the optional stripping step or the isomerization reaction step or both in counter-current manner. In the isomerization step, the pressure varies in the range of 20-150 bar, preferably in the range of 20-100 bar, the temperature being between 200 and 500° C., preferably between 300 and 400° C. In the isomerization step, isomerization catalysts known in the art may be used. Suitable isomerization catalysts contain molecular sieve and/or a metal from Group VII and/or a carrier. Preferably, the isomerization catalyst contains SAPO-11 or SAPO41 or ZSM-22 or ZSM-23 or ferrierite and Pt, Pd or Ni and Al₂O₃ or SiO₂. Typical isomerization catalysts are, for example, Pt/SAPO-11/Al₂O₃, Pt/ZSM-22/Al₂O₃, Pt/ZSM-23/Al₂O₃ and Pt/SAPO-11/SiO₂. The isomerization step and the HDO step may be carried out in the same pressure vessel or in separate pressure vessels. Optional prehydrogenation may be carried out in a separate pressure vessel or in the same pressure vessel as the HDO and isomerization steps.

Thus, in one embodiment, the product of one or more chemical reactions is an alkane mixture that comprises HRJ-5. In another embodiment, the product of the one or more chemical reactions is an alkane mixture that comprises ASTM D1655 jet fuel. In some embodiments, the composition conforming to the specification of ASTM 1655 jet fuel has a sulfur content that is less than 10 ppm. In other embodiments, the composition conforming to the specification of ASTM 1655 jet fuel has a T10 value of the distillation curve of less than 205° C. In another embodiment, the composition conforming to the specification of ASTM 1655 jet fuel has a final boiling point (FBP) of less than 300° C. In another embodiment, the composition conforming to the specification of ASTM 1655 jet fuel has a flash point of at least 38° C. In another embodiment, the composition conforming to the specification of ASTM 1655 jet fuel has a density between 775K/M³ and 840K/M³. In yet another embodiment, the composition conforming to the specification of ASTM 1655 jet fuel has a freezing point that is below −47° C. In another embodiment, the composition conforming to the specification of ASTM 1655 jet fuel has a net Heat of Combustion that is at least 42.8 MJ/K. In another embodiment, the composition conforming to the specification of ASTM 1655 jet fuel has a hydrogen content that is at least 13.4 mass %. In another embodiment, the composition conforming to the specification of ASTM 1655 jet fuel has a thermal stability, as tested by quantitative gravimetric JFTOT at 260° C., which is below 3 mm of Hg. In another embodiment, the composition conforming to the specification of ASTM 1655 jet fuel has an existent gum that is below 7 mg/dl.

Thus, the present invention discloses a variety of methods in which chemical modification of microalgal lipid is undertaken to yield products useful in a variety of industrial and other applications. Examples of processes for modifying oil produced by the methods disclosed herein include, but are not limited to, hydrolysis of the oil, hydroprocessing of the oil, and esterification of the oil. Other chemical modification of microalgal lipid include, without limitation, epoxidation, oxidation, hydrolysis, sulfations, sulfonation, ethoxylation, propoxylation, amidation, and saponification. The modification of the microalgal oil produces basic oleochemicals that can be further modified into selected derivative oleochemicals for a desired function. In a manner similar to that described above with reference to fuel producing processes, these chemical modifications can also be performed on oils generated from the microbial cultures described herein. Examples of basic oleochemicals include, but are not limited to, soaps, fatty acids, fatty esters, fatty alcohols, fatty nitrogen compounds including fatty amides, fatty acid methyl esters, and glycerol. Examples of derivative oleochemicals include, but are not limited to, fatty nitriles, esters, dimer acids, quats (including betaines), surfactants, fatty alkanolamides, fatty alcohol sulfates, resins, emulsifiers, fatty alcohols, olefins, drilling muds, polyols, polyurethanes, polyacrylates, rubber, candles, cosmetics, metallic soaps, soaps, alpha-sulphonated methyl esters, fatty alcohol sulfates, fatty alcohol ethoxylates, fatty alcohol ether sulfates, imidazolines, surfactants, detergents, esters, quats (including betaines), ozonolysis products, fatty amines, fatty alkanolamides, ethoxysulfates, monoglycerides, diglycerides, triglycerides (including medium chain triglycerides), lubricants, hydraulic fluids, greases, dielectric fluids, mold release agents, metal working fluids, heat transfer fluids, other functional fluids, industrial chemicals (e.g., cleaners, textile processing aids, plasticizers, stabilizers, additives), surface coatings, paints and lacquers, electrical wiring insulation, and higher alkanes. Other derivatives include fatty amidoamines, amidoamine carboxylates, amidoamine oxides, amidoamine oxide carboxylates, amidoamine esters, ethanolamine amides, sulfonates, amidoamine sulfonates, diamidoamine dioxides, sulfonated alkyl ester alkoxylates, betaines, quarternized diamidoamine betaines, and sulfobetaines.

Hydrolysis of the fatty acid constituents from the glycerolipids produced by the methods of the invention yields free fatty acids that can be derivatized to produce other useful chemicals. Hydrolysis occurs in the presence of water and a catalyst which may be either an acid or a base. The liberated free fatty acids can be derivatized to yield a variety of products, as reported in the following: U.S. Pat. No. 5,304,664 (Highly sulfated fatty acids); U.S. Pat. No. 7,262,158 (Cleansing compositions); U.S. Pat. No. 7,115,173 (Fabric softener compositions); U.S. Pat. No. 6,342,208 (Emulsions for treating skin); U.S. Pat. No. 7,264,886 (Water repellant compositions); U.S. Pat. No. 6,924,333 (Paint additives); U.S. Pat. No. 6,596,768 (Lipid-enriched ruminant feedstock); and U.S. Pat. No. 6,380,410 (Surfactants for detergents and cleaners).

In some methods, the first step of chemical modification may be hydroprocessing to saturate double bonds, followed by deoxygenation at elevated temperature in the presence of hydrogen and a catalyst. In other methods, hydrogenation and deoxygenation may occur in the same reaction. In still other methods deoxygenation occurs before hydrogenation. Isomerization may then be optionally performed, also in the presence of hydrogen and a catalyst. Finally, gases and naphtha components can be removed if desired. For example, see U.S. Pat. No. 5,475,160 (hydrogenation of triglycerides); U.S. Pat. No. 5,091,116 (deoxygenation, hydrogenation and gas removal); U.S. Pat. No. 6,391,815 (hydrogenation); and U.S. Pat. No. 5,888,947 (isomerization).

In some embodiments of the invention, the triglyceride oils are partially or completely deoxygenated. The deoxygenation reactions form desired products, including, but not limited to, fatty acids, fatty alcohols, polyols, ketones, and aldehydes. In general, without being limited by any particular theory, the deoxygenation reactions involve a combination of various different reaction pathways, including without limitation: hydrogenolysis, hydrogenation, consecutive hydrogenation-hydrogenolysis, consecutive hydrogenolysis-hydrogenation, and combined hydrogenation-hydrogenolysis reactions, resulting in at least the partial removal of oxygen from the fatty acid or fatty acid ester to produce reaction products, such as fatty alcohols, that can be easily converted to the desired chemicals by further processing. For example, in one embodiment, a fatty alcohol may be converted to olefins through FCC reaction or to higher alkanes through a condensation reaction.

One such chemical modification is hydrogenation, which is the addition of hydrogen to double bonds in the fatty acid constituents of glycerolipids or of free fatty acids. The hydrogenation process permits the transformation of liquid oils into semi-solid or solid fats, which may be more suitable for specific applications.

Hydrogenation of oil produced by the methods described herein can be performed in conjunction with one or more of the methods and/or materials provided herein, as reported in the following: U.S. Pat. No. 7,288,278 (Food additives or medicaments); U.S. Pat. No. 5,346,724 (Lubrication products); U.S. Pat. No. 5,475,160 (Fatty alcohols); U.S. Pat. No. 5,091,116 (Edible oils); U.S. Pat. No. 6,808,737 (Structural fats for margarine and spreads); U.S. Pat. No. 5,298,637 (Reduced-calorie fat substitutes); U.S. Pat. No. 6,391,815 (Hydrogenation catalyst and sulfur adsorbent); U.S. Pat. Nos. 5,233,099 and 5,233,100 (Fatty alcohols); U.S. Pat. No. 4,584,139 (Hydrogenation catalysts); U.S. Pat. No. 6,057,375 (Foam suppressing agents); and U.S. Pat. No. 7,118,773 (Edible emulsion spreads).

One skilled in the art will recognize that various processes may be used to hydrogenate carbohydrates. One suitable method includes contacting the carbohydrate with hydrogen or hydrogen mixed with a suitable gas and a catalyst under conditions sufficient in a hydrogenation reactor to form a hydrogenated product. The hydrogenation catalyst generally can include Cu, Re, Ni, Fe, Co, Ru, Pd, Rh, Pt, Os, Ir, and alloys or any combination thereof, either alone or with promoters such as W, Mo, Au, Ag, Cr, Zn, Mn, Sn, B, P, Bi, and alloys or any combination thereof. Other effective hydrogenation catalyst materials include either supported nickel or ruthenium modified with rhenium. In an embodiment, the hydrogenation catalyst also includes any one of the supports, depending on the desired functionality of the catalyst. The hydrogenation catalysts may be prepared by methods known to those of ordinary skill in the art.

In some embodiments the hydrogenation catalyst includes a supported Group VIII metal catalyst and a metal sponge material (e.g., a sponge nickel catalyst). Raney nickel provides an example of an activated sponge nickel catalyst suitable for use in this invention. In other embodiment, the hydrogenation reaction in the invention is performed using a catalyst comprising a nickel-rhenium catalyst or a tungsten-modified nickel catalyst. One example of a suitable catalyst for the hydrogenation reaction of the invention is a carbon-supported nickel-rhenium catalyst.

In an embodiment, a suitable Raney nickel catalyst may be prepared by treating an alloy of approximately equal amounts by weight of nickel and aluminum with an aqueous alkali solution, e.g., containing about 25 weight % of sodium hydroxide. The aluminum is selectively dissolved by the aqueous alkali solution resulting in a sponge shaped material comprising mostly nickel with minor amounts of aluminum. The initial alloy includes promoter metals (i.e., molybdenum or chromium) in the amount such that about 1 to 2 weight % remains in the formed sponge nickel catalyst. In another embodiment, the hydrogenation catalyst is prepared using a solution of ruthenium (III) nitrosylnitrate, ruthenium (III) chloride in water to impregnate a suitable support material. The solution is then dried to form a solid having a water content of less than about 1% by weight. The solid may then be reduced at atmospheric pressure in a hydrogen stream at 300° C. (uncalcined) or 400° C. (calcined) in a rotary ball furnace for 4 hours. After cooling and rendering the catalyst inert with nitrogen, 5% by volume of oxygen in nitrogen is passed over the catalyst for 2 hours.

In certain embodiments, the catalyst described includes a catalyst support. The catalyst support stabilizes and supports the catalyst. The type of catalyst support used depends on the chosen catalyst and the reaction conditions. Suitable supports for the invention include, but are not limited to, carbon, silica, silica-alumina, zirconia, titania, ceria, vanadia, nitride, boron nitride, heteropolyacids, hydroxyapatite, zinc oxide, chromia, zeolites, carbon nanotubes, carbon fullerene and any combination thereof.

The catalysts used in this invention can be prepared using conventional methods known to those in the art. Suitable methods may include, but are not limited to, incipient wetting, evaporative impregnation, chemical vapor deposition, wash-coating, magnetron sputtering techniques, and the like.

The conditions for which to carry out the hydrogenation reaction will vary based on the type of starting material and the desired products. One of ordinary skill in the art, with the benefit of this disclosure, will recognize the appropriate reaction conditions. In general, the hydrogenation reaction is conducted at temperatures of 80° C. to 250° C., and preferably at 90° C. to 200° C., and most preferably at 100° C. to 150° C. In some embodiments, the hydrogenation reaction is conducted at pressures from 500 KPa to 14000 KPa.

The hydrogen used in the hydrogenolysis reaction of the current invention may include external hydrogen, recycled hydrogen, in situ generated hydrogen, and any combination thereof. As used herein, the term “external hydrogen” refers to hydrogen that does not originate from the biomass reaction itself, but rather is added to the system from another source.

In some embodiments of the invention, it is desirable to convert the starting carbohydrate to a smaller molecule that will be more readily converted to desired higher hydrocarbons. One suitable method for this conversion is through a hydrogenolysis reaction. Various processes are known for performing hydrogenolysis of carbohydrates. One suitable method includes contacting a carbohydrate with hydrogen or hydrogen mixed with a suitable gas and a hydrogenolysis catalyst in a hydrogenolysis reactor under conditions sufficient to form a reaction product comprising smaller molecules or polyols. As used herein, the term “smaller molecules or polyols” includes any molecule that has a smaller molecular weight, which can include a smaller number of carbon atoms or oxygen atoms than the starting carbohydrate. In an embodiment, the reaction products include smaller molecules that include polyols and alcohols. Someone of ordinary skill in the art would be able to choose the appropriate method by which to carry out the hydrogenolysis reaction.

In some embodiments, a 5 and/or 6 carbon sugar or sugar alcohol may be converted to propylene glycol, ethylene glycol, and glycerol using a hydrogenolysis catalyst. The hydrogenolysis catalyst may include Cr, Mo, W, Re, Mn, Cu, Cd, Fe, Co, Ni, Pt, Pd, Rh, Ru, Ir, Os, and alloys or any combination thereof, either alone or with promoters such as Au, Ag, Cr, Zn, Mn, Sn, Bi, B, O, and alloys or any combination thereof. The hydrogenolysis catalyst may also include a carbonaceous pyropolymer catalyst containing transition metals (e.g., chromium, molybdenum, tungsten, rhenium, manganese, copper, cadmium) or Group VIII metals (e.g., iron, cobalt, nickel, platinum, palladium, rhodium, ruthenium, iridium, and osmium). In certain embodiments, the hydrogenolysis catalyst may include any of the above metals combined with an alkaline earth metal oxide or adhered to a catalytically active support. In certain embodiments, the catalyst described in the hydrogenolysis reaction may include a catalyst support as described above for the hydrogenation reaction.

The conditions for which to carry out the hydrogenolysis reaction will vary based on the type of starting material and the desired products. One of ordinary skill in the art, with the benefit of this disclosure, will recognize the appropriate conditions to use to carry out the reaction. In general, they hydrogenolysis reaction is conducted at temperatures of 110° C. to 300° C., and preferably at 170° C. to 220° C., and most preferably at 200° C. to 225° C. In some embodiments, the hydrogenolysis reaction is conducted under basic conditions, preferably at a pH of 8 to 13, and even more preferably at a pH of 10 to 12. In some embodiments, the hydrogenolysis reaction is conducted at pressures in a range between 60 KPa and 16500 KPa, and preferably in a range between 1700 KPa and 14000 KPa, and even more preferably between 4800 KPa and 11000 KPa.

The hydrogen used in the hydrogenolysis reaction of the current invention can include external hydrogen, recycled hydrogen, in situ generated hydrogen, and any combination thereof.

In some embodiments, the reaction products discussed above may be converted into higher hydrocarbons through a condensation reaction in a condensation reactor. In such embodiments, condensation of the reaction products occurs in the presence of a catalyst capable of forming higher hydrocarbons. While not intending to be limited by theory, it is believed that the production of higher hydrocarbons proceeds through a stepwise addition reaction including the formation of carbon-carbon, or carbon-oxygen bond. The resulting reaction products include any number of compounds containing these moieties, as described in more detail below.

In certain embodiments, suitable condensation catalysts include an acid catalyst, a base catalyst, or an acid/base catalyst. As used herein, the term “acid/base catalyst” refers to a catalyst that has both an acid and a base functionality. In some embodiments the condensation catalyst can include, without limitation, zeolites, carbides, nitrides, zirconia, alumina, silica, aluminosilicates, phosphates, titanium oxides, zinc oxides, vanadium oxides, lanthanum oxides, yttrium oxides, scandium oxides, magnesium oxides, cerium oxides, barium oxides, calcium oxides, hydroxides, heteropolyacids, inorganic acids, acid modified resins, base modified resins, and any combination thereof. In some embodiments, the condensation catalyst can also include a modifier. Suitable modifiers include La, Y, Sc, P, B, Bi, Li, Na, K, Rb, Cs, Mg, Ca, Sr, Ba, and any combination thereof. In some embodiments, the condensation catalyst can also include a metal. Suitable metals include Cu, Ag, Au, Pt, Ni, Fe, Co, Ru, Zn, Cd, Ga, In, Rh, Pd, Ir, Re, Mn, Cr, Mo, W, Sn, Os, alloys, and any combination thereof.

In certain embodiments, the catalyst described in the condensation reaction may include a catalyst support as described above for the hydrogenation reaction. In certain embodiments, the condensation catalyst is self-supporting. As used herein, the term “self-supporting” means that the catalyst does not need another material to serve as support. In other embodiments, the condensation catalyst in used in conjunction with a separate support suitable for suspending the catalyst. In an embodiment, the condensation catalyst support is silica.

The conditions under which the condensation reaction occurs will vary based on the type of starting material and the desired products. One of ordinary skill in the art, with the benefit of this disclosure, will recognize the appropriate conditions to use to carry out the reaction. In some embodiments, the condensation reaction is carried out at a temperature at which the thermodynamics for the proposed reaction are favorable. The temperature for the condensation reaction will vary depending on the specific starting polyol or alcohol. In some embodiments, the temperature for the condensation reaction is in a range from 80° C. to 500° C., and preferably from 125° C. to 450° C., and most preferably from 125° C. to 250° C. In some embodiments, the condensation reaction is conducted at pressures in a range between 0 Kpa to 9000 KPa, and preferably in a range between 0 KPa and 7000 KPa, and even more preferably between 0 KPa and 5000 KPa.

The higher alkanes formed by the invention include, but are not limited to, branched or straight chain alkanes that have from 4 to 30 carbon atoms, branched or straight chain alkenes that have from 4 to 30 carbon atoms, cycloalkanes that have from 5 to 30 carbon atoms, cycloalkenes that have from 5 to 30 carbon atoms, aryls, fused aryls, alcohols, and ketones. Suitable alkanes include, but are not limited to, butane, pentane, pentene, 2-methylbutane, hexane, hexene, 2-methylpentane, 3-methylpentane, 2,2,-dimethylbutane, 2,3-dimethylbutane, heptane, heptene, octane, octene, 2,2,4-trimethylpentane, 2,3-dimethyl hexane, 2,3,4-trimethylpentane, 2,3-dimethylpentane, nonane, nonene, decane, decene, undecane, undecene, dodecane, dodecene, tridecane, tridecene, tetradecane, tetradecene, pentadecane, pentadecene, nonyldecane, nonyldecene, eicosane, eicosene, uneicosane, uneicosene, doeicosane, doeicosene, trieicosane, trieicosene, tetraeicosane, tetraeicosene, and isomers thereof. Some of these products may be suitable for use as fuels.

In some embodiments, the cycloalkanes and the cycloalkenes are unsubstituted. In other embodiments, the cycloalkanes and cycloalkenes are mono-substituted. In still other embodiments, the cycloalkanes and cycloalkenes are multi-substituted. In the embodiments comprising the substituted cycloalkanes and cycloalkenes, the substituted group includes, without limitation, a branched or straight chain alkyl having 1 to 12 carbon atoms, a branched or straight chain alkylene having 1 to 12 carbon atoms, a phenyl, and any combination thereof. Suitable cycloalkanes and cycloalkenes include, but are not limited to, cyclopentane, cyclopentene, cyclohexane, cyclohexene, methyl-cyclopentane, methyl-cyclopentene, ethyl-cyclopentane, ethyl-cyclopentene, ethyl-cyclohexane, ethyl-cyclohexene, isomers and any combination thereof.

In some embodiments, the aryls formed are unsubstituted. In another embodiment, the aryls formed are mono-substituted. In the embodiments comprising the substituted aryls, the substituted group includes, without limitation, a branched or straight chain alkyl having 1 to 12 carbon atoms, a branched or straight chain alkylene having 1 to 12 carbon atoms, a phenyl, and any combination thereof. Suitable aryls for the invention include, but are not limited to, benzene, toluene, xylene, ethyl benzene, para xylene, meta xylene, and any combination thereof.

The alcohols produced in the invention have from 4 to 30 carbon atoms. In some embodiments, the alcohols are cyclic. In other embodiments, the alcohols are branched. In another embodiment, the alcohols are straight chained. Suitable alcohols for the invention include, but are not limited to, butanol, pentanol, hexanol, heptanol, octanol, nonanol, decanol, undecanol, dodecanol, tridecanol, tetradecanol, pentadecanol, hexadecanol, heptyldecanol, octyldecanol, nonyldecanol, eicosanol, uneicosanol, doeicosanol, trieicosanol, tetraeicosanol, and isomers thereof.

The ketones produced in the invention have from 4 to 30 carbon atoms. In an embodiment, the ketones are cyclic. In another embodiment, the ketones are branched. In another embodiment, the ketones are straight chained. Suitable ketones for the invention include, but are not limited to, butanone, pentanone, hexanone, heptanone, octanone, nonanone, decanone, undecanone, dodecanone, tridecanone, tetradecanone, pentadecanone, hexadecanone, heptyldecanone, octyldecanone, nonyldecanone, eicosanone, uneicosanone, doeicosanone, trieicosanone, tetraeicosanone, and isomers thereof.

Another such chemical modification is interesterification. Naturally produced glycerolipids do not have a uniform distribution of fatty acid constituents. In the context of oils, interesterification refers to the exchange of acyl radicals between two esters of different glycerolipids. The interesterification process provides a mechanism by which the fatty acid constituents of a mixture of glycerolipids can be rearranged to modify the distribution pattern. Interesterification is a well-known chemical process, and generally comprises heating (to about 200° C.) a mixture of oils for a period (e.g., 30 minutes) in the presence of a catalyst, such as an alkali metal or alkali metal alkylate (e.g., sodium methoxide). This process can be used to randomize the distribution pattern of the fatty acid constituents of an oil mixture, or can be directed to produce a desired distribution pattern. This method of chemical modification of lipids can be performed on materials provided herein, such as microbial biomass with a percentage of dry cell weight as lipid at least 20%.

Directed interesterification, in which a specific distribution pattern of fatty acids is sought, can be performed by maintaining the oil mixture at a temperature below the melting point of some TAGs which might occur. This results in selective crystallization of these TAGs, which effectively removes them from the reaction mixture as they crystallize. The process can be continued until most of the fatty acids in the oil have precipitated, for example. A directed interesterification process can be used, for example, to produce a product with a lower calorie content via the substitution of longer-chain fatty acids with shorter-chain counterparts. Directed interesterification can also be used to produce a product with a mixture of fats that can provide desired melting characteristics and structural features sought in food additives or products (e.g., margarine) without resorting to hydrogenation, which can produce unwanted trans isomers.

Interesterification of oils produced by the methods described herein can be performed in conjunction with one or more of the methods and/or materials, or to produce products, as reported in the following: U.S. Pat. No. 6,080,853 (Nondigestible fat substitutes); U.S. Pat. No. 4,288,378 (Peanut butter stabilizer); U.S. Pat. No. 5,391,383 (Edible spray oil); U.S. Pat. No. 6,022,577 (Edible fats for food products); U.S. Pat. No. 5,434,278 (Edible fats for food products); U.S. Pat. No. 5,268,192 (Low calorie nut products); U.S. Pat. No. 5,258,197 (Reduce calorie edible compositions); U.S. Pat. No. 4,335,156 (Edible fat product); U.S. Pat. No. 7,288,278 (Food additives or medicaments); U.S. Pat. No. 7,115,760 (Fractionation process); U.S. Pat. No. 6,808,737 (Structural fats); U.S. Pat. No. 5,888,947 (Engine lubricants); U.S. Pat. No. 5,686,131 (Edible oil mixtures); and U.S. Pat. No. 4,603,188 (Curable urethane compositions).

In one embodiment in accordance with the invention, transesterification of the oil, as described above, is followed by reaction of the transesterified product with polyol, as reported in U.S. Pat. No. 6,465,642, to produce polyol fatty acid polyesters. Such an esterification and separation process may comprise the steps as follows: reacting a lower alkyl ester with polyol in the presence of soap; removing residual soap from the product mixture; water-washing and drying the product mixture to remove impurities; bleaching the product mixture for refinement; separating at least a portion of the unreacted lower alkyl ester from the polyol fatty acid polyester in the product mixture; and recycling the separated unreacted lower alkyl ester.

Transesterification can also be performed on microbial biomass with short chain fatty acid esters, as reported in U.S. Pat. No. 6,278,006. In general, transesterification may be performed by adding a short chain fatty acid ester to an oil in the presence of a suitable catalyst and heating the mixture. In some embodiments, the oil comprises about 5% to about 90% of the reaction mixture by weight. In some embodiments, the short chain fatty acid esters can be about 10% to about 50% of the reaction mixture by weight. Non-limiting examples of catalysts include base catalysts, sodium methoxide, acid catalysts including inorganic acids such as sulfuric acid and acidified clays, organic acids such as methane sulfonic acid, benzenesulfonic acid, and toluenesulfonic acid, and acidic resins such as Amberlyst 15. Metals such as sodium and magnesium, and metal hydrides also are useful catalysts.

Another such chemical modification is hydroxylation, which involves the addition of water to a double bond resulting in saturation and the incorporation of a hydroxyl moiety. The hydroxylation process provides a mechanism for converting one or more fatty acid constituents of a glycerolipid to a hydroxy fatty acid. Hydroxylation can be performed, for example, via the method reported in U.S. Pat. No. 5,576,027. Hydroxylated fatty acids, including castor oil and its derivatives, are useful as components in several industrial applications, including food additives, surfactants, pigment wetting agents, defoaming agents, water proofing additives, plasticizing agents, cosmetic emulsifying and/or deodorant agents, as well as in electronics, pharmaceuticals, paints, inks, adhesives, and lubricants. One example of how the hydroxylation of a glyceride may be performed is as follows: fat may be heated, preferably to about 30-50° C. combined with heptane and maintained at temperature for thirty minutes or more; acetic acid may then be added to the mixture followed by an aqueous solution of sulfuric acid followed by an aqueous hydrogen peroxide solution which is added in small increments to the mixture over one hour; after the aqueous hydrogen peroxide, the temperature may then be increased to at least about 60° C. and stirred for at least six hours; after the stirring, the mixture is allowed to settle and a lower aqueous layer formed by the reaction may be removed while the upper heptane layer formed by the reaction may be washed with hot water having a temperature of about 60° C.; the washed heptane layer may then be neutralized with an aqueous potassium hydroxide solution to a pH of about 5 to 7 and then removed by distillation under vacuum; the reaction product may then be dried under vacuum at 100° C. and the dried product steam-deodorized under vacuum conditions and filtered at about 50° to 60° C. using diatomaceous earth.

Hydroxylation of microbial oils produced by the methods described herein can be performed in conjunction with one or more of the methods and/or materials, or to produce products, as reported in the following: U.S. Pat. No. 6,590,113 (Oil-based coatings and ink); U.S. Pat. No. 4,049,724 (Hydroxylation process); U.S. Pat. No. 6,113,971 (Olive oil butter); U.S. Pat. No. 4,992,189 (Lubricants and lube additives); U.S. Pat. No. 5,576,027 (Hydroxylated milk); and U.S. Pat. No. 6,869,597 (Cosmetics).

Hydroxylated glycerolipids can be converted to estolides. Estolides consist of a glycerolipid in which a hydroxylated fatty acid constituent has been esterified to another fatty acid molecule. Conversion of hydroxylated glycerolipids to estolides can be carried out by warming a mixture of glycerolipids and fatty acids and contacting the mixture with a mineral acid, as described by Isbell et al., JAOCS 71(2):169-174 (1994). Estolides are useful in a variety of applications, including without limitation those reported in the following: U.S. Pat. No. 7,196,124 (Elastomeric materials and floor coverings); U.S. Pat. No. 5,458,795 (Thickened oils for high-temperature applications); U.S. Pat. No. 5,451,332 (Fluids for industrial applications); U.S. Pat. No. 5,427,704 (Fuel additives); and U.S. Pat. No. 5,380,894 (Lubricants, greases, plasticizers, and printing inks).

Another such chemical modification is olefin metathesis. In olefin metathesis, a catalyst severs the alkylidene carbons in an alkene (olefin) and forms new alkenes by pairing each of them with different alkylidine carbons. The olefin metathesis reaction provides a mechanism for processes such as truncating unsaturated fatty acid alkyl chains at alkenes by ethenolysis, cross-linking fatty acids through alkene linkages by self-metathesis, and incorporating new functional groups on fatty acids by cross-metathesis with derivatized alkenes.

In conjunction with other reactions, such as transesterification and hydrogenation, olefin metathesis can transform unsaturated glycerolipids into diverse end products. These products include glycerolipid oligomers for waxes; short-chain glycerolipids for lubricants; homo- and hetero-bifunctional alkyl chains for chemicals and polymers; short-chain esters for biofuel; and short-chain hydrocarbons for jet fuel. Olefin metathesis can be performed on triacylglycerols and fatty acid derivatives, for example, using the catalysts and methods reported in U.S. Pat. No. 7,119,216, US Patent Pub. No. 2010/0160506, and U.S. Patent Pub. No. 2010/0145086.

Olefin metathesis of bio-oils generally comprises adding a solution of Ru catalyst at a loading of about 10 to 250 ppm under inert conditions to unsaturated fatty acid esters in the presence (cross-metathesis) or absence (self-metathesis) of other alkenes. The reactions are typically allowed to proceed from hours to days and ultimately yield a distribution of alkene products. One example of how olefin metathesis may be performed on a fatty acid derivative is as follows: A solution of the first generation Grubbs Catalyst (dichloro[2(1-methylethoxy-α-O)phenyl]methylene-α-C] (tricyclohexyl-phosphine) in toluene at a catalyst loading of 222 ppm may be added to a vessel containing degassed and dried methyl oleate. Then the vessel may be pressurized with about 60 psig of ethylene gas and maintained at or below about 30° C. for 3 hours, whereby approximately a 50% yield of methyl 9-decenoate may be produced.

Olefin metathesis of oils produced by the methods described herein can be performed in conjunction with one or more of the methods and/or materials, or to produce products, as reported in the following: Patent App. PCT/US07/081427 (α-olefin fatty acids) and U.S. patent application Ser. No. 12/281,938 (petroleum creams), Ser. No. 12/281,931 (paintball gun capsules), Ser. No. 12/653,742 (plasticizers and lubricants), Ser. No. 12/422,096 (bifunctional organic compounds), and Ser. No. 11/795,052 (candle wax).

Other chemical reactions that can be performed on microbial oils include reacting triacylglycerols with a cyclopropanating agent to enhance fluidity and/or oxidative stability, as reported in U.S. Pat. No. 6,051,539; manufacturing of waxes from triacylglycerols, as reported in U.S. Pat. No. 6,770,104; and epoxidation of triacylglycerols, as reported in “The effect of fatty acid composition on the acrylation kinetics of epoxidized triacylglycerols”, Journal of the American Oil Chemists' Society, 79:1, 59-63, (2001) and Free Radical Biology and Medicine, 37:1, 104-114 (2004).

The generation of oil-bearing microbial biomass for fuel and chemical products as described above results in the production of delipidated biomass meal. Delipidated meal is a byproduct of preparing algal oil and is useful as animal feed for farm animals, e.g., ruminants, poultry, swine and aquaculture. The resulting meal, although of reduced oil content, still contains high quality proteins, carbohydrates, fiber, ash, residual oil and other nutrients appropriate for an animal feed. Because the cells are predominantly lysed by the oil separation process, the delipidated meal is easily digestible by such animals. Delipidated meal can optionally be combined with other ingredients, such as grain, in an animal feed. Because delipidated meal has a powdery consistency, it can be pressed into pellets using an extruder or expander or another type of machine, which are commercially available.

The invention, having been described in detail above, is exemplified in the following examples, which are offered to illustrate, but not to limit, the claimed invention.

### EXAMPLES

### Example 1

### Fatty Acid Analysis by Fatty Acid Methyl Ester Detection

Lipid samples were prepared from dried biomass. 20-40 mg of dried biomass was resuspended in 2 mL of 5% H₂SO₄ in MeOH, and 200 ul of toluene containing an appropriate amount of a suitable internal standard (C19:0) was added. The mixture was sonicated briefly to disperse the biomass, then heated at 70-75° C. for 3.5 hours. 2 mL of heptane was added to extract the fatty acid methyl esters, followed by addition of 2 mL of 6% K₂CO₃ (aq) to neutralize the acid. The mixture was agitated vigorously, and a portion of the upper layer was transferred to a vial containing Na₂SO₄ (anhydrous) for gas chromatography analysis using standard FAME GC/FID (fatty acid methyl ester gas chromatography flame ionization detection) methods. Fatty acid profiles reported below were determined by this method.

### Example 2

### Engineering Microorganisms for Fatty Acid and Sn-2 Profiles Increased in Lauric Acid Through Exogenous LPAAT Expression

This example describes the use of recombinant polynucleotides that encode a C. nucifera 1-acyl-sn-glycerol-3-phosphate acyltransferase (Cn LPAAT) enzyme to engineer a microorganism in which the fatty acid profile and the sn-2 profile of the transformed microorganism has been enriched in lauric acid.

A classically mutagenized strain of Prototheca moriformis (UTEX 1435), Strain A, was initially transformed with the plasmid construct pSZ1283 according to biolistic transformation methods as described in PCT/US2009/066141, PCT/US2009/066142, PCT/US2011/038463, PCT/US2011/038464, and PCT/US2012/023696. pSZ1283, described in PCT/US2011/038463, PCT/US2011/038464, and PCT/US2012/023696 hereby incorporated by reference, comprised the coding sequence of the Cuphea wrightii FATB2 (CwTE2) thioesterase (SEQ ID NO: 10), 5′ (SEQ ID NO: 1) and 3′ (SEQ ID NO: 2) homologous recombination targeting sequences (flanking the construct) to the 6S genomic region for integration into the nuclear genome, and a S. cerevisiae suc2 sucrose invertase coding region (SEQ ID NO: 4), to express the protein sequence given in SEQ ID NO: 3, under the control of C. reinhardtii β-tubulin promoter/5′UTR (SEQ ID NO: 5) and Chlorella vulgaris nitrate reductase 3′ UTR (SEQ ID NO: 6). This S. cerevisiae suc2 expression cassette is listed as SEQ ID NO: 7 and served as a selectable marker. The CwTE2 protein coding sequence to express the protein sequence given in SEQ ID NO: 11, was under the control of the P. moriformis Amt03 promoter/5′UTR (SEQ ID NO: 8) and C. vulgaris nitrate reductase 3′UTR. The protein coding regions of CwTE2 and suc2 were codon optimized to reflect the codon bias inherent in P. moriformis UTEX 1435 nuclear genes as described in PCT/US2009/066141, PCT/US2009/066142, PCT/US2011/038463, PCT/US2011/038464, and PCT/US2012/023696.

Upon transformation of pSZ1283 into Strain A, positive clones were selected on agar plates with sucrose as the sole carbon source. Primary transformants were then clonally purified and a single transformant, Strain B, was selected for further genetic modification. This genetically engineered strain was transformed with plasmid construct pSZ2046 to interrupt the pLoop genomic locus of Strain B. Construct pSZ2046 comprised the coding sequence of the C. nucifera 1-acyl-sn-glycerol-3-phosphate acyltransferase (Cn LPAAT) enzyme (SEQ ID NO: 12), 5′ (SEQ ID NO: 13) and 3′ (SEQ ID NO: 14) homologous recombination targeting sequences (flanking the construct) to the pLoop genomic region for integration into the nuclear genome, and a neomycin resistance protein-coding sequence under the control of C. reinhardtii β-tubulin promoter/5′UTR (SEQ ID NO: 5), and Chlorella vulgaris nitrate reductase 3′ UTR (SEQ ID NO: 6). This NeoR expression cassette is listed as SEQ ID NO: 15 and served as a selectable marker. The Cn LPAAT protein coding sequence was under the control of the P. moriformis Amt03 promoter/5′UTR (SEQ ID NO: 8) and C. vulgaris nitrate reductase 3′UTR. The protein coding regions of Cn LPAAT and NeoR were codon optimized to reflect the codon bias inherent in P. moriformis UTEX 1435 nuclear genes as described in PCT/US2009/066141, PCT/US2009/066142, PCT/US2011/038463, PCT/US2011/038464, and PCT/US2012/023696. The amino acid sequence of Cn LPAAT is provided as SEQ ID NO: 16.

Upon transformation of pSZ2046 into Strain B, thereby generating Strain C, positive clones were selected on agar plates comprising G418 (Geneticin). Individual transformants were clonally purified and grown at pH 7.0 under conditions suitable for lipid production as detailed in PCT/US2009/066141, PCT/US2009/066142, PCT/US2011/038463, PCT/US2011/038464, and PCT/US2012/023696. Lipid samples were prepared from dried biomass from each transformant and fatty acid profiles from these samples were analyzed using standard fatty acid methyl ester gas chromatography flame ionization (FAME GC/FID) detection methods as described in Example 1. The fatty acid profiles (expressed as Area % of total fatty acids) of P. moriformis UTEX 1435 (U1) grown on glucose as a sole carbon source, untransformed Strain B and five pSZ2046 positive transformants (Strain C, 1-5) are presented in Table 10.

TABLE 10
Effect of LPAAT expression on fatty acid profiles
of transformed Prototheca moriformis (UTEX 1435)
comprising a mid-chain preferring thioesterase.

| Area % | | Strain | Strain | Strain | Strain | Strain | Strain |
|---|---|---|---|---|---|---|---|
| Fatty acid | U1 | B | C-1 | C-2 | C-3 | C-4 | C-5 |
| C10:0 | 0.01 | 5.53 | 11.37 | 11.47 | 10.84 | 11.13 | 11.12 |
| C12:0 | 0.04 | 31.04 | 46.63 | 46.47 | 45.84 | 45.80 | 45.67 |
| C14:0 | 1.27 | 15.99 | 15.14 | 15.12 | 15.20 | 15.19 | 15.07 |
| C16:0 | 27.20 | 12.49 | 7.05 | 7.03 | 7.30 | 7.20 | 7.19 |
| C18:0 | 3.85 | 1.30 | 0.71 | 0.72 | 0.74 | 0.74 | 0.74 |
| C18:l | 58.70 | 24.39 | 10.26 | 10.41 | 10.95 | 11.31 | 11.45 |
| C18:2 | 7.18 | 7.79 | 7.05 | 6.93 | 7.30 | 6.88 | 7.01 |
| C10-C12 | 0.50 | 36.57 | 58.00 | 57.94 | 56.68 | 56.93 | 56.79 |

As shown in Table 10, the fatty acid profile of Strain B expressing CwTE2 showed increased composition of C10:0, C12:0, and C14:0 fatty acids and a decrease in C16:0, C18:0, and C18:1 fatty acids relative to the fatty acid profile of the untransformed UTEX 1435 strain. The impact of additional genetic modification on the fatty acid profile of the transformed strains, namely the expression of CnLPAAT in Strain B, is a still further increase in the composition of C10:0 and C12:0 fatty acids, a still further decrease in C16:0, C18:0, and C18:1 fatty acids, but no significant effect on the C14:0 fatty acid composition. These data indicate that the CnLPAAT shows substrate preference in the context of a microbial host organism.

The untransformed P. moriformis Strain A is characterized by a fatty acid profile comprising less than 0.5% C12 fatty acids and less than 1% C10-C12 fatty acids. In contrast, the fatty acid profile of Strain B expressing a C. wrightii thioesterase comprised 31% C12:0 fatty acids, with C10-C12 fatty acids comprising greater than 36% of the total fatty acids. Further, fatty acid profiles of Strain C, expressing a higher plant thioesterase and a CnLPAAT enzyme, comprised between 45.67% and 46.63% C12:0 fatty acids, with C10-C12% fatty acids comprising between 71 and 73% of total fatty acids. The result of expressing an exogenous thioesterase was a 62-fold increase in the percentage of C12 fatty acid present in the engineered microbe. The result of expressing an exogenous thioesterase and exogenous LPAAT was a 92-fold increase in the percentage of C12 fatty acids present in the engineered microbe.

The TAG fraction of oil samples extracted from Strains A, B, and C were analyzed for the sn-2 profile of their triacylglycerides. The TAGs were extracted and processed, and analyzed as in Example 1. The fatty acid composition and the sn-2 profiles of the TAG fraction of oil extracted from Strains A, B, and C (expressed as Area % of total fatty acids) are presented in Table 11. Values not reported are indicated as “n.r.”

TABLE 11
Effect of LPAAT expression on the fatty acid composition and the
sn-2 profile of TAGs produced from transformed Prototheca moriformis
(UTEX 1435) comprising a mid-chain preferring thioesterase.

| | Strain | | | | | |
|---|---|---|---|---|---|---|
| | Strain A | | Strain B | | Strain C | |
| | (untransformed) | | (pSZ1500) | | (pSZ1500 + pSZ2046) | |
| Area % | | sn-2 | | sn-2 | | sn-2 |
| fatty acid | FA | profile | FA | profile | FA | profile |
| C10:0 | n.r. | n.r. | 11.9 | 14.2 | 12.4 | 7.1 |
| C12:0 | n.r. | n.r. | 42.4 | 25 | 47.9 | 52.8 |
| C14:0 | 1.0 | 0.6 | 12 | 10.4 | 13.9 | 9.1 |
| C16:0 | 23.9 | 1.6 | 7.2 | 1.3 | 6.1 | 0.9 |
| C18:0 | 3.7 | 0.3 | n.r | n.r. | 0.8 | 0.3 |
| C18:1 | 64.3 | 90.5 | 18.3 | 36.6 | 9.9 | 17.5 |
| C18:2 | 4.5 | 5.8 | 5.8 | 10.8 | 6.5 | 10 |
| C18:3 | n.r. | n.r. | n.r. | n.r. | 1.1 | 1.6 |

As shown in Table 11, the fatty acid composition of triglycerides (TAGs) isolated from Strain B expressing CwTE2 was increased for C10:0, C12:0, and C14:0 fatty acids and decrease in C16:0 and C18:1 fatty acids relative to the fatty acid profile of TAGs isolated from untransformed Strain A. The impact of additional genetic modification on the fatty acid profile of the transformed strains, namely the expression of CnLPAAT, was a still further increase in the composition of C10:0 and C12:0 fatty acids, a still further decrease in C16:0, C18:0, and C18:1 fatty acids, but no significant effect on the C14:0 fatty acid composition. These data indicate that expression of the exogenous CnLPAAT improves the midchain fatty acid profile of transformed microbes.

The untransformed P. moriformis Strain A is characterized by an sn-2 profile of about 0.6% C14, about 1.6% C16:0, about 0.3% C18:0, about 90% C18:1, and about 5.8% C18:2. In contrast to Strain A, Strain B, expressing a C. wrightii thioesterase is characterized by an sn-2 profile that is higher in midchain fatty acids and lower in long chain fatty acids. C12 fatty acids comprised 25% of the sn-2 profile of Strain B. The impact of additional genetic modification on the sn-2 profile of the transformed strains, namely the expression of CnLPAAT, was still a further increase in C12 fatty acids (from 25% to 52.8%), a decrease in C18:1 fatty acids (from 36.6% to 17.5%), and a decrease in C10:0 fatty acids. (The sn-2 profile composition of C14:0 and C16:0 fatty acids was relatively similar for Strains B and C.)

These data demonstrate the utility and effectiveness of polynucleotides permitting exogenous LPAAT expression to alter the fatty acid profile of engineered microorganisms, and in particular in increasing the concentration of C10:0 and C12:0 fatty acids in microbial cells. These data further demonstrate the utility and effectiveness of polynucleotides permitting exogenous thioesterase and exogenous LPAAT expression to alter the sn-2 profile of TAGs produced by microbial cells, in particular in increasing the C12 composition of sn-2 profiles and decreasing the C18:1 composition of sn-2 profiles.

### Example 3

### Analysis of Regiospecific Profile

LC/MS TAG distribution analyses were carried out using a Shimadzu Nexera ultra high performance liquid chromatography system that included a SIL-30AC autosampler, two LC-30AD pumps, a DGU-20A5 in-line degasser, and a CTO-20A column oven, coupled to a Shimadzu LCMS 8030 triple quadrupole mass spectrometer equipped with an APCI source. Data was acquired using a Q3 scan of m/z 350-1050 at a scan speed of 1428 u/sec in positive ion mode with the CID gas (argon) pressure set to 230 kPa. The APCI, desolvation line, and heat block temperatures were set to 300, 250, and 200° C., respectively, the flow rates of the nebulizing and drying gases were 3.0 L/min and 5.0 L/min, respectively, and the interface voltage was 4500 V. Oil samples were dissolved in dichloromethane-methanol (1:1) to a concentration of 5 mg/mL, and 0.8 μL of sample was injected onto Shimadzu Shim-pack XR-ODS III (2.2 μm, 2.0×200 mm) maintained at 30° C. A linear gradient from 30% dichloromethane-2-propanol (1:1)/acetonitrile to 51% dichloromethane-2-propanol (1:1)/acetonitrile over 27 minutes at 0.48 mL/min was used for chromatographic separations.

### Example 4

### Engineering Microorganisms for Increased Production of Erucic Acid Through Elongase or Beta-Ketoacyl-CoA Synthase Overexpression

In an embodiment of the present invention, a recombinant polynucleotide transformation vector operable to express an exogenous elongase or beta-ketoacyl-CoA synthase in an optionally plastidic oleaginous microbe is constructed and employed to transform Prototheca moriformis (UTEX 1435) according to the biolistic transformation methods as described in PCT/US2009/066141, PCT/US2009/066142, PCT/US2011/038463, PCT/US2011/038464, and PCT/US2012/023696 to obtain a cell increased for production of erucic acid. The transformation vector includes a protein coding region to overexpress an elongase or beta-ketoacyl-CoA synthase such as those listed in Table 8, promoter and 3′UTR control sequences to regulate expression of the exogenous gene, 5′ and 3′ homologous recombination targeting sequences targeting the recombinant polynucleotides for integration into the P. moriformis (UTEX 1435) nuclear genome, and nucleotides operable to express a selectable marker. The protein-coding sequences of the transformation vector are codon-optimized for expression in P. moriformis (UTEX 1435) as described in PCT/US2009/066141, PCT/US2009/066142, PCT/US2011/038463, PCT/US2011/038464, and PCT/US2012/023696. Recombinant polynucleotides encoding promoters, 3′ UTRs, and selectable markers operable for expression in P. moriformis (UTEX 1435) are disclosed herein and in PCT/US2009/066141, PCT/US2009/066142, PCT/US2011/038463, PCT/US2011/038464, and PCT/US2012/023696.

Upon transformation of the transformation vector into P. moriformis (UTEX 1435) or a classically-mutagenized strain of P. moriformis (UTEX 1435), positive clones are selected on agar plates. Individual transformants are clonally purified and cultivated under heterotrophic conditions suitable for lipid production as detailed in PCT/US2009/066141, PCT/US2009/066142, PCT/US2011/038463, PCT/US2011/038464, and PCT/US2012/023696. Lipid samples are prepared from dried biomass from each transformant and fatty acid profiles from these samples are analyzed using fatty acid methyl ester gas chromatography flame ionization (FAME GC/FID) detection methods as described in Example 1. As a result of these manipulations, the cell may exhibit an increase in erucic acid of at least 5, 10, 15, or 20 fold.

The transgenic CuPSR23 LPAAT2 strains (D1520A-E) show a significant increase in the accumulation of C10:0, C12:0, and C14:0 fatty acids with a concomitant decrease in C18:1 and C18:2. The transgenic CuPSR23 LPAAT3 strains (D1521A-E) show a significant increase in the accumulation of C10:0, C12:0, and C14:0 fatty acids with a concomitant decrease in C18:1. The expression of the CuPSR23 LPAAT in these transgenic lines appears to be directly responsible for the increased accumulation of mid-chain fatty acids in general, and especially laurates. While the transgenic lines show a shift from longer chain fatty acids (C16:0 and above) to mid-chain fatty acids, the shift is targeted predominantly to C10:0 and C12:0 fatty acids with a slight effect on C14:0 fatty acids. The data presented also show that co-expression of the LPAAT2 and LPAAT3 genes from Cuphea PSR23 and the FATB2 from C. wrightii (expressed in the strain Strain B) have an additive effect on the accumulation of C12:0 fatty acids.

Our results suggest that the LPAAT enzymes from Cuphea PSR23 are active in the algal strains derived from UTEX 1435. These results also demonstrate that the enzyme functions in conjunction with the heterologous FatB2 acyl-ACP thioesterase enzyme expressed in Strain B, which is derived from Cuphea wrightii.

The transgenic CuPSR23 LPAATx strains (D1542A-E) show a significant decrease in the accumulation of C10:0, C12:0, and C14:0 fatty acids relative to the parent, Strain B, with a concomitant increase in C16:0, C18:0, C18:1 and C18:2. The expression of the CuPSR23 LPAATx gene in these transgenic lines appears to be directly responsible for the decreased accumulation of mid-chain fatty acids (C10-C14) and the increased accumulation of C16:0 and C18 fatty acids, with the most pronounced increase observed in palmitates (C16:0). The data presented also show that despite the expression of the midchain specific FATB2 from C. wrightii (present in Strain B), the expression of CuPSR23 LPAATx appears to favor incorporation of longer chain fatty acids into TAGs.

Our results suggest that the LPAATx enzyme from Cuphea PSR23 is active in the algal strains derived from UTEX 1435. Contrary to Cuphea PSR23 LPAAT2 and LPAAT3, which increase mid-chain fatty acid levels, CuPSR23 LPAATx leads to increased C16:0 and C18:0 levels. These results demonstrate that the different LPAATs derived from CuPSR23 (LPAAT2, LPAAT3, and LPAATx) exhibit different fatty acid specificities in Strain B as judged by their effects on overall fatty acid levels.

### Example 5

### Production of Eicosenoic and Erucic Fatty Acids

In this example we demonstrate that expression of heterologous fatty acid elongase (FAE), also known as 3-ketoacyl-CoA synthase (KCS), genes from Cramble abyssinica (CaFAE, Accession No: AY793549), Lunaria annua (LaFAE, ACJ61777), and Cardamine graeca (CgFAE, ACJ61778) leads to production of very long chain monounsaturated fatty acids such as eicosenoic (20:1^{Δ11}) and erucic (22:1^{Δ13}) acids in classically mutagenized derivative of UTEX 1435, Strain Z. On the other hand a putative FAE gene from Tropaeolum majus (TmFAE, ABD77097) and two FAE genes from Brassica napus (BnFAE1, AAA96054 and BnFAE2, AAT65206), while resulting in modest increase in eicosenoic (20:1^{Δ11}), produced no detectable erucic acid in STRAIN Z. Interestingly the unsaturated fatty acid profile obtained with heterologous expression of BnFAE1 in STRAIN Z resulted in noticeable increase in Docosadienoic acid (22:2n6). All the genes were codon optimized to reflect UTEX 1435 codon usage. These results suggest that CaFAE, LaFAE or CgFAE genes encode condensing enzymes involved in the biosynthesis of very long-chain utilizing monounsaturated and saturated acyl substrates, with specific capability for improving the eicosenoic and erucic acid content.

Construct Used for the Expression of the Cramble abyssinica Fatty Acid Elongase (CaFAE) in P. moriformis (UTEX 1435 Strain Z)—[pSZ3070]:

In this example STRAIN Z strains, transformed with the construct pSZ3070, were generated, which express sucrose invertase (allowing for their selection and growth on medium containing sucrose) and C. abyssinica FAE gene. Construct pSZ3070 introduced for expression in STRAIN Z can be written as 6S::CrTUB2-ScSUC2-Cvnr:PmAmt03-CaFAE-Cvnr::6S.

The sequence of the transforming DNA is provided below. Relevant restriction sites in the construct are indicated in lowercase, bold, and are from 5′-3′ BspQI, KpnI, XbaI, MfeI, BamHI, EcoRI, SpeI, AflII, SacI, BspQI, respectively. BspQI sites delimit the 5′ and 3′ ends of the transforming DNA. Bold, lowercase sequences represent genomic DNA from STRAIN Z that permit targeted integration at the 6S locus via homologous recombination. Proceeding in the 5′ to 3′ direction, the C. reinhardtii β-tubulin promoter driving the expression of the Saccharomyces cerevisiae SUC2 gene (encoding sucrose hydrolyzing activity, thereby permitting the strain to grow on sucrose) is indicated by lowercase, boxed text. The initiator ATG and terminator TGA for SUC2 are indicated by uppercase italics, while the coding region is indicated with lowercase italics. The Chlorella vulgaris nitrate reductase (NR) gene 3′ UTR is indicated by lowercase underlined text followed by an endogenous AMTS promoter of P. moriformis, indicated by boxed italicized text. The Initiator ATG and terminator TGA codons of the CaFAE are indicated by uppercase, bold italics, while the remainder of the gene is indicated by bold italics. The C. vulgaris nitrate reductase 3′ UTR is again indicated by lowercase underlined text followed by the STRAIN Z 6S genomic region indicated by bold, lowercase text. The final construct was sequenced to ensure correct reading frames and targeting sequences.

Nucleotide sequence of transforming DNA contained in plasmid pSZ3070:
(SEQ ID NO: 35)
gctcttcgccgccgccactcctgctcgagcgcgcccgcgcgtgcgccgccagcgccttggccttttcgccgcgctcgtgcgcgtcgctgatgt
ccatcaccaggtccatgaggtctgccttgcgccggctgagccactgcttcgtccgggcggccaagaggagcatgagggaggactcctggt
ccagggtcctgacgtggtcgcggctctgggagcgggccagcatcatctggctctgccgcaccgaggccgcctccaactggtcctccagca
gccgcagtcgccgccgaccctggcagaggaagacaggtgaggggggtatgaattgtacagaacaaccacgagccttgtctaggcagaa
tccctaccagtcatggctttacctggatgacggcctgcgaacagctgtccagcgaccctcgctgccgccgcttctcccgcacgcttctttcca
gcaccgtgatggcgcgagccagcgccgcacgctggcgctgcgcttcgccgatctgaggacagtcggggaactctgatcagtctaaacccc
cttgcgcgttagtgttgccatcctttgcagaccggtgagagccgacttgttgtgcgccaccccccacaccacctcctcccagaccaattctgt

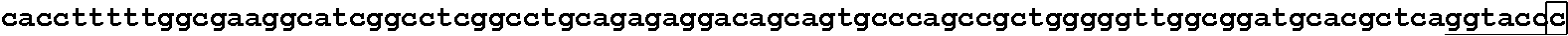

cacctttttggcgaaggcatcggcctcggcctgcagagaggacagcagtgcccagccgctgggggttggcggatgcacgctcaggtacc

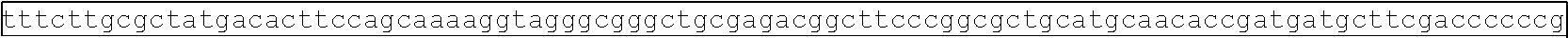

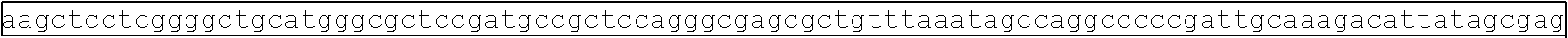

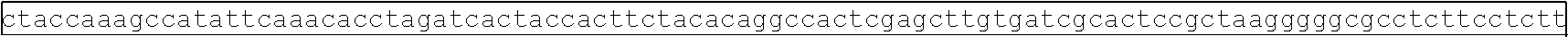

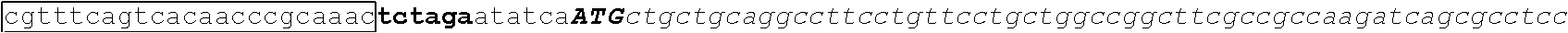

atgacgaacgagacgtccgaccgccccctggtgcacttcacccccaacaagggctggatgaacgaccccaacggcctgtggtacgacgag
aaggacgccaagtggcacctgtacttccagtacaacccgaacgacaccgtctggggacgcccttgttctggggccacgccacgtccgacg
acctgaccaactgggaggaccagcccatcgccatcgccccgaagcgcaacgactccggcgccttctccggctccatggtggtggactacaa
caacacctccggcttcttcaacgacaccatcgacccgcgccagcgctgcgtggccatctggacctacaacaccccggagtccgaggagcagt
acatctcctacagcctggacggcggctacaccttcaccgagtaccagaagaaccccgtgctggccgccaactccacccagttccgcgacccg
aaggtcttctggtacgagccctcccagaagtggatcatgaccgcggccaagtcccaggactacaagatcgagatctactcctccgacgacctg
aagtcctggaagctggagtccgcgttcgccaacgagggcttcctcggctaccagtacgagtgccccggcctgatcgaggtccccaccgagca
ggaccccagcaagtcctactgggtgatgttcatctccatcaaccccggcgccccggccggcggctccttcaaccagtacttcgtcggcagcttc
aacggcacccacttcgaggccttcgacaaccagtcccgcgtggtggacttcggcaaggactactacgccctgcagaccttcttcaacaccgac
ccgacctacgggagcgccctgggcatcgcgtgggcctccaactgggagtactccgccttcgtgcccaccaacccctggcgctcctccatgtcc
ctcgtgcgcaagttctccctcaacaccgagtaccaggccaacccggagacggagctgatcaacctgaaggccgagccgatcctgaacatca
gcaacgccggcccctggagccggttcgccaccaacaccacgttgacgaaggccaacagctacaacgtcgacctgtccaacagcaccggca
ccctggagttcgagctggtgtacgccgtcaacaccacccagacgatctccaagtccgtgttcgcggacctctccctctggttcaagggcctgga
ggaccccgaggagtacctccgcatgggcttcgaggtgtccgcgtcctccttcttcctggaccgcgggaacagcaaggtgaagttcgtgaagga
gaacccctacttcaccaaccgcatgagcgtgaacaaccagcccttcaagagcgagaacgacctgtcctactacaaggtgtacggcttgctgg
accagaacatcctggagctgtacttcaacgacggcgacgtcgtgtccaccaacacctacttcatgaccaccgggaacgccctgggctccgtg

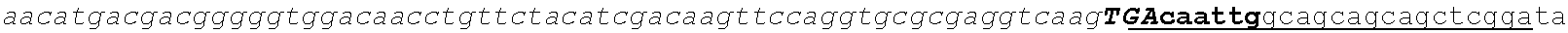

gtatcgacacactctggacctggtcgtgtgatggactgttgccgccacacttgctgccttgacctgtgaatatccctgccgcttttatcaaacagcctc
agtgtgtttgatcttgtgtgtacgcgcttttgcgagttgctagctgcttgtgctatttgcgaataccacccccagcatccccttccctcgtttcatatcgctt
gcatcccaaccgcaacttatctacgctgtcctgctatccctcagcgctgctcctgctcctgctcactgcccctcgcacagccttggtttgggctccgcc
tgtattctcctggtactgcaacctgtaaaccagcactgcaatgctgatgcacgggaagtagtgggatgggaacacaaatggaggatcccgcgtctc
gaacagagcgcgcagaggaacgctgaaggtctcgcctctgtcgcacctcagcgcggcatacaccacaataaccacctgacgaatgcgcttggtt
cttcgtccattagcgaagcgtccggttcacacacgtgccacgttggcgaggtggcaggtgacaatgatcggtggagctgatggtcgaaacgttcac

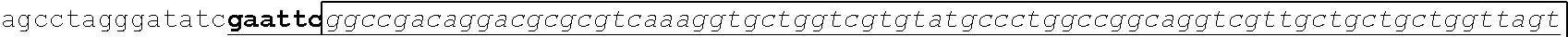

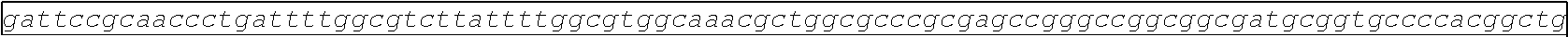

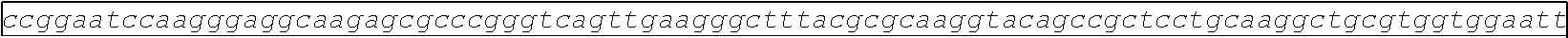

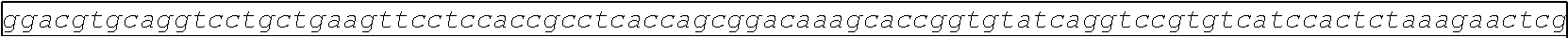

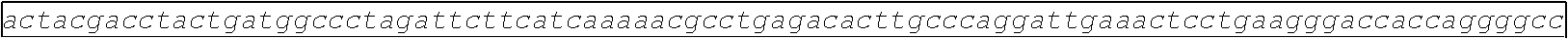

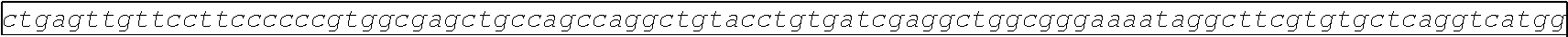

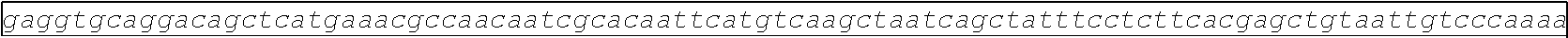

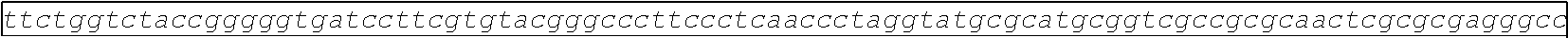

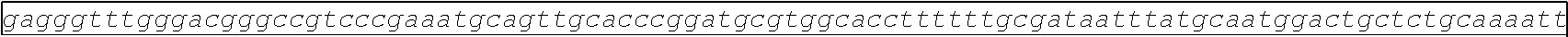

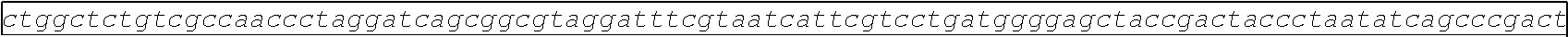

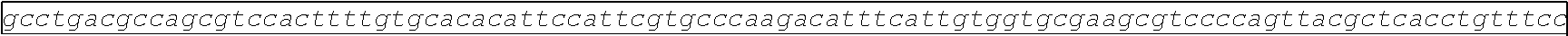

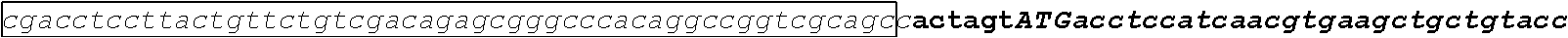

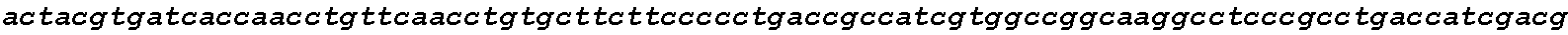

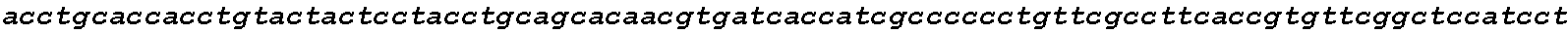

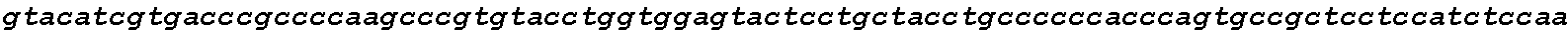

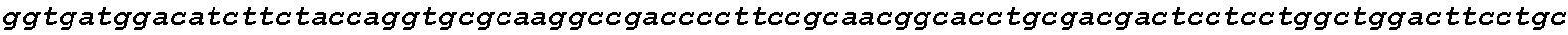

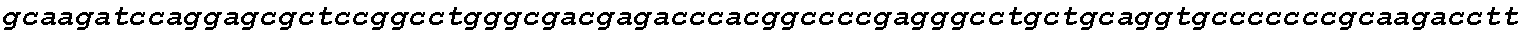

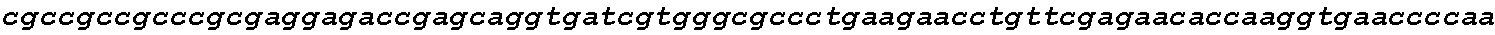

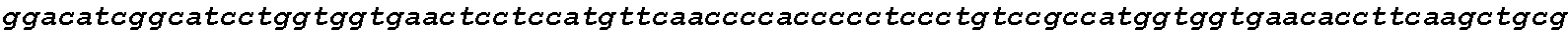

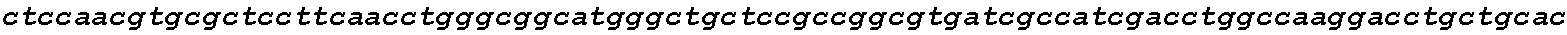

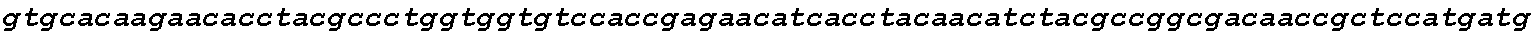

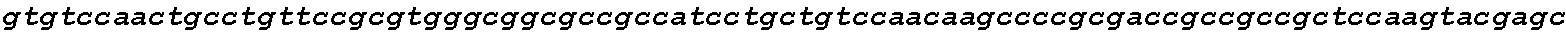

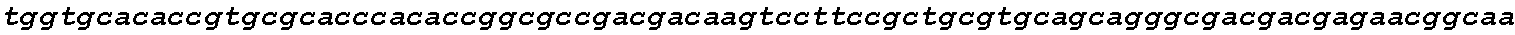

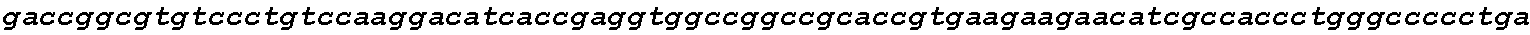

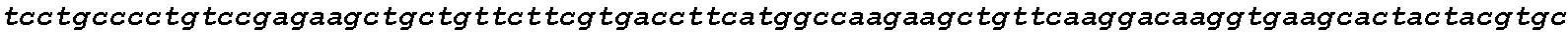

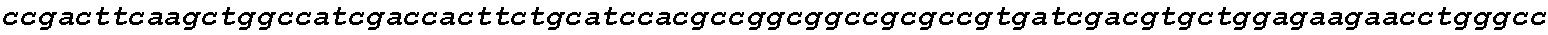

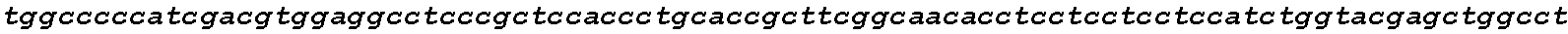

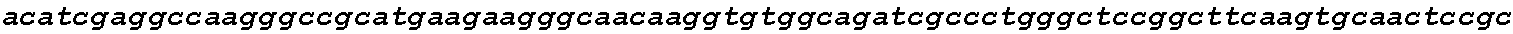

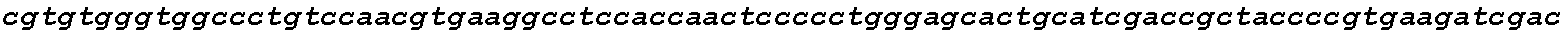

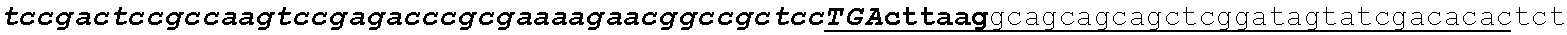

ggacgctggtcgtgtgatggactgttgccgccacacttgctgccttgacctgtgaatatccctgccgcttttatcaaacagcctcagtgtgtttgatcttg
tgtgtacgcgcttttgcgagttgctagctgcttgtgctatttgcgaataccacccccagcatccccaccctcgtttcatatcgcttgcatcccaaccgca
acttatctacgctgtcctgctatccctcagcgctgctcctgctcctgctcactgcccctcgcacagccttggtttgggctccgcctgtattctcctggtac
tgcaacctgtaaaccagcactgcaatgctgatgcacgggaagtagtgggatgggaacacaaatggaaagcttaattaagagctcttgttttccaga
aggagttgctccttgagcctttcattctcagcctcgataacctccaaagccgctctaattgtggagggggttcgaatttaaaagcttggaatg
ttggttcgtgcgtctggaacaagcccagacttgttgctcactgggaaaaggaccatcagctccaaaaaacttgccgctcaaaccgcgtacc
tctgctttcgcgcaatctgccctgttgaaatcgccaccacattcatattgtgacgcttgagcagtctgtaattgcctcagaatgtggaatcatc
tgccccctgtgcgagcccatgccaggcatgtcgcgggcgaggacacccgccactcgtacagcagaccattatgctacctcacaatagttca
taacagtgaccatatttctcgaagctccccaacgagcacctccatgctctgagtggccaccccccggccctggtgcttgcggagggcaggt
caaccggcatggggctaccgaaatccccgaccggatcccaccacccccgcgatgggaagaatctctccccgggatgtgggcccaccacc
agcacaacctgctggcccaggcgagcgtcaaaccataccacacaaatatccttggcatcggccctgaattccttctgccgctctgctacccg
gtgcttctgtccgaagcaggggttgctagggatcgctccgagtccgcaaacccttgtcgcgtggcggggcttgttcgagcttgaagagc

Constructs Used for the Expression of the FAE Genes from Higher Plants in Strain Z:

In addition to the CaFAE gene (pSZ3070), LaFAE (pSZ3071) from Lunaria annua, CgFAE (pSZ3072) from Cardamine graeca, TmFAE (pSZ3067) Tropaeolum majus and BnFAE1 (pSZ3068) and BnFAE2 (pSZ3069) genes from Brassica napus have been constructed for expression in STRAIN Z. These constructs can be described as:

pSZ3071—6S::CrTUB2-ScSUC2-Cvnr:PmAmt03-LaFAE-Cvnr::6S
pSZ3072—6S::CrTUB2-ScSUC2-Cvnr:PmAmt03-CgFAE-Cvnr::6S
pSZ3067—6S::CrTUB2-ScSUC2-Cvnr:PmAmt03-TmFAE-Cvnr::6S
pSZ3068—6S::CrTUB2-ScSUC2-Cvnr:PmAmt03-BnFAE1-Cvnr::6S
pSZ3069—6S::CrTUB2-ScSUC2-Cvnr:PmAmt03-BnFAE2-Cvnr::6S

All these constructs have the same vector backbone; selectable marker, promoters, and 3′ utr as pSZ3070, differing only in the respective FAE genes. Relevant restriction sites in these constructs are also the same as in pSZ3070. The sequences of LaFAE, CgFAE, TmFAE, BnFAE1 and BnFAE2 are shown below. Relevant restriction sites as bold text including SpeI and AflII are shown 5′-3′ respectively.

Nucleotide sequence of LaFAE contained in pSZ3071:
(SEQ ID NO:36)

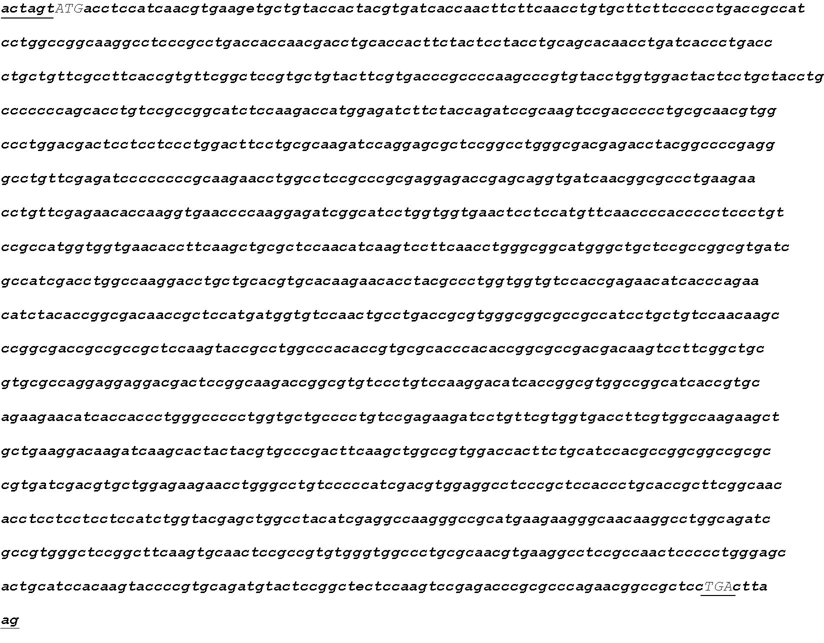

Nucleotide sequence of CgFAE contained in pSZ3072:
(SEQ ID NO:37)

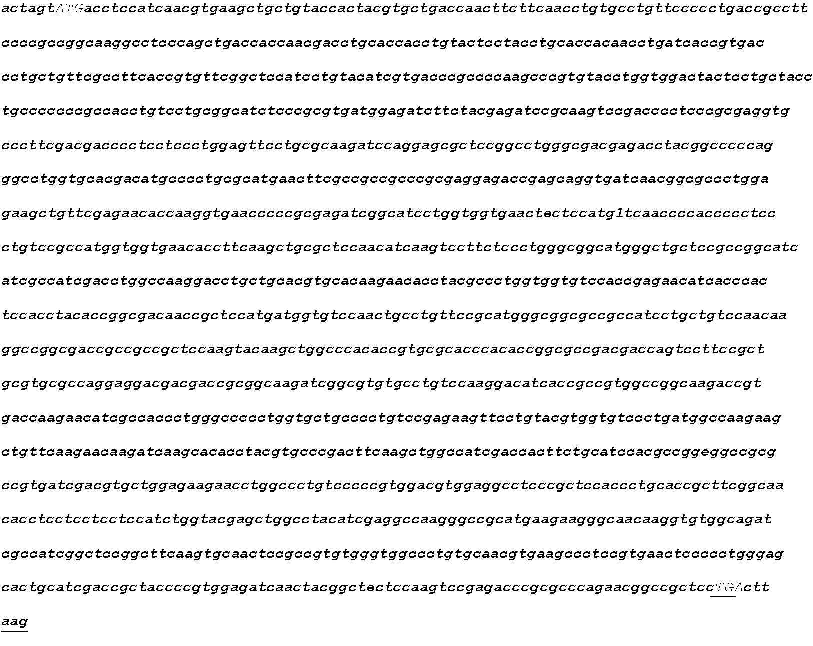

Nucleotide sequence of TmFAE contained in pSZ3067:
(SEQ ID NO:38)

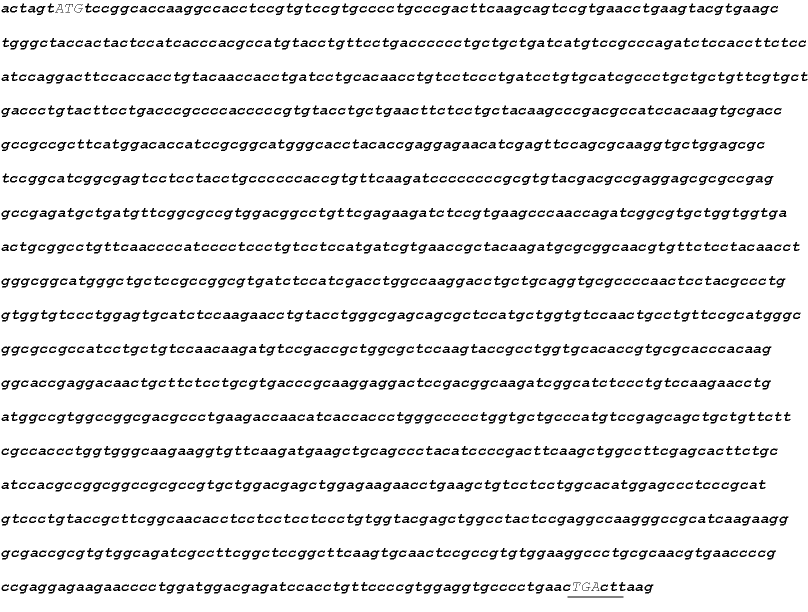

Nucleotide sequence of BnFAE1 contained in pSZ3068:
(SEQ ID NO:39)

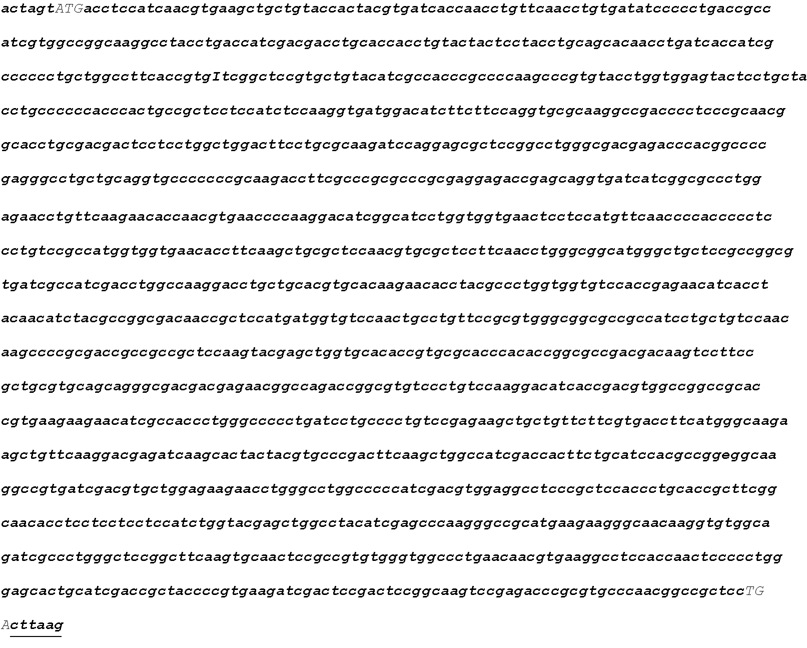

Nucleotide sequence of BnFAE2 contained in pSZ3069:
(SEQ ID NO:40)

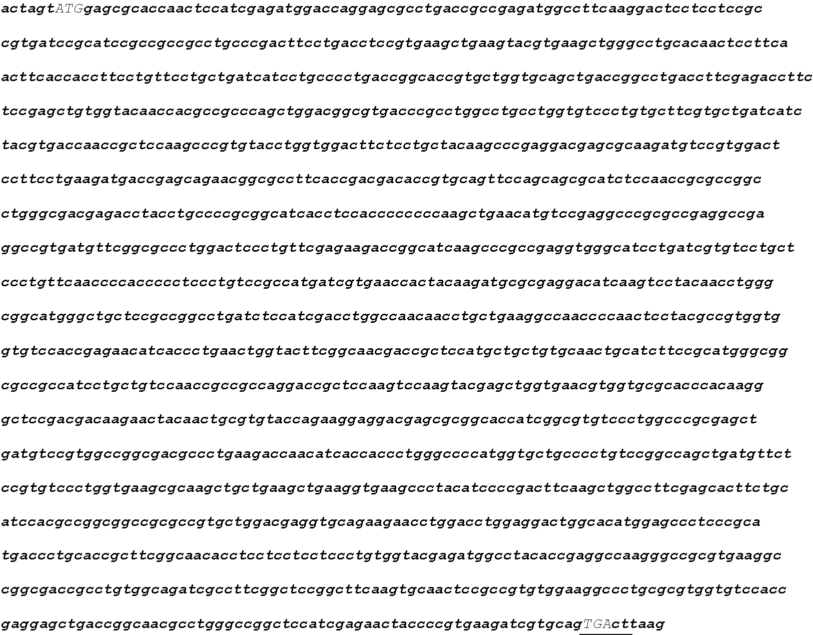

To determine their impact on fatty acid profiles, the above constructs containing various heterologous FAE genes, driven by the PmAMT3 promoter, were transformed independently into STRAIN Z.

Primary transformants were clonally purified and grown under low-nitrogen lipid production conditions at pH7.0 (all the plasmids require growth at pH 7.0 to allow for maximal FAE gene expression when driven by the pH regulated PmAMT03 promoter). The resulting profiles from a set of representative clones arising from transformations with pSZ3070, pSZ3071, pSZ3072, pSZ3067, pSZ3068 and pSZ3069 into STRAIN Z are shown in Tables 12-17, respectively, below.

All the transgenic STRAIN Z strains expressing heterologous FAE genes show an increased accumulation of C20:1 and C22:1 fatty acid (see Tables 12-17). The increase in eicosenoic (20:1^{Δ11}) and erucic (22:1^{Δ13}) acids levels over the wildtype is consistently higher than the wildtype levels. Additionally, the unsaturated fatty acid profile obtained with heterologous expression of BnFAE1 in STRAIN Z resulted in noticeable increase in Docosadienoic acid (C22:2n6). Protein alignment of aforementioned FAE expressed in STRAIN Z is shown in Figure.

TABLE 12
Unsaturated fatty acid profile in STRAIN Z and representative derivative
transgenic lines transformed with pSZ3070 (CaFAE) DNA.

| Sample ID | C18:1 | C18:2 | C18:3a | C20:1 | C22:1 | C22:2n6 | C22:5 |
|---|---|---|---|---|---|---|---|
| STRAIN Z; | 51.49 | 9.13 | 0.65 | 4.35 | 1.24 | 0.11 | 0.00 |
| T588; | | | | | | | |
| D1828-20 | | | | | | | |
| STRAIN Z; | 55.59 | 7.65 | 0.50 | 3.78 | 0.85 | 0.00 | 0.13 |
| T588; | | | | | | | |
| D1828-23 | | | | | | | |
| STRAIN Z; | 54.70 | 7.64 | 0.50 | 3.44 | 0.85 | 0.09 | 0.00 |
| T588; | | | | | | | |
| D1828-43 | | | | | | | |
| STRAIN Z; | 52.43 | 7.89 | 0.59 | 2.72 | 0.73 | 0.00 | 0.00 |
| T588; | | | | | | | |
| D1828-12 | | | | | | | |
| STRAIN Z; | 56.02 | 7.12 | 0.52 | 3.04 | 0.63 | 0.10 | 0.11 |
| T588; | | | | | | | |
| D1828-19 | | | | | | | |
| Cntrl | 57.99 | 6.62 | 0.56 | 0.19 | 0.00 | 0.06 | 0.05 |
| STRAIN Z | | | | | | | |
| pH 7 | | | | | | | |
| Cntrl | 57.70 | 7.08 | 0.54 | 0.11 | 0.00 | 0.05 | 0.05 |
| STRAIN Z | | | | | | | |
| pH 5 | | | | | | | |

TABLE 13
Unsaturated fatty acid profile in STRAIN Z and representative derivative
transgenic lines transformed with pSZ3071 (LaFAE) DNA.

| Sample ID | C18:1 | C18:2 | C18:3 a | C20:1 | C22:1 | C22:2n6 | C22:5 |
|---|---|---|---|---|---|---|---|
| STRAIN Z; | 54.66 | 7.04 | 0.52 | 1.82 | 0.84 | 0.12 | 0.09 |
| T588; | | | | | | | |
| D1829-36 | | | | | | | |
| STRAIN Z; | 56.27 | 6.72 | 0.51 | 1.70 | 0.72 | 0.09 | 0.00 |
| T588; | | | | | | | |
| D1829-24 | | | | | | | |
| STRAIN Z; | 56.65 | 8.36 | 0.54 | 2.04 | 0.67 | 0.00 | 0.00 |
| T588; | | | | | | | |
| D1829-11 | | | | | | | |
| STRAIN Z; | 55.57 | 7.71 | 0.53 | 0.10 | 0.66 | 0.00 | 0.00 |
| T588; | | | | | | | |
| D1829-35 | | | | | | | |
| STRAIN Z; | 56.03 | 7.06 | 0.54 | 1.54 | 0.51 | 0.06 | 0.08 |
| T588; | | | | | | | |
| D1829-42 | | | | | | | |
| Cntrl | 57.70 | 7.08 | 0.54 | 0.11 | 0.00 | 0.06 | 0.05 |
| STRAIN Z | | | | | | | |
| pH 7 | | | | | | | |
| Cntrl | 57.99 | 6.62 | 0.56 | 0.19 | 0.00 | 0.05 | 0.05 |
| STRAIN Z | | | | | | | |
| pH 5 | | | | | | | |

TABLE 14
Unsaturated fatty acid profile in STRAIN Z and representative derivative
transgenic lines transformed with pSZ3072 (CgFAE) DNA.

| Sample ID | C18:1 | C18:2 | C18:3 a | C20:1 | C22:1 | C22:2n6 | C22:5 |
|---|---|---|---|---|---|---|---|
| STRAIN Z; | 57.74 | 7.79 | 0.52 | 1.61 | 0.25 | 0.11 | 0.05 |
| T588; | | | | | | | |
| D1830-47 | | | | | | | |
| STRAIN Z; | 58.06 | 7.39 | 0.55 | 1.64 | 0.22 | 0.07 | 0.06 |
| T588; | | | | | | | |
| D1830-16 | | | | | | | |
| STRAIN Z; | 57.77 | 6.86 | 0.51 | 1.34 | 0.19 | 0.09 | 0.00 |
| T588; | | | | | | | |
| D1830-12 | | | | | | | |
| STRAIN Z; | 58.45 | 7.54 | 0.49 | 1.65 | 0.19 | 0.06 | 0.00 |
| T588; | | | | | | | |
| D1830-37 | | | | | | | |
| STRAIN Z; | 57.10 | 7.28 | 0.56 | 1.43 | 0.19 | 0.07 | 0.00 |
| T588; | | | | | | | |
| D1830-44 | | | | | | | |
| Cntrl | 57.70 | 7.08 | 0.54 | 0.11 | 0.00 | 0.06 | 0.05 |
| STRAIN Z | | | | | | | |
| pH 7 | | | | | | | |
| Cntrl | 57.99 | 6.62 | 0.56 | 0.19 | 0.00 | 0.05 | 0.05 |
| STRAIN Z | | | | | | | |
| pH 5 | | | | | | | |

TABLE 15
Unsaturated fatty acid profile in Strain AR and representative
derivative transgenic lines transformed with pSZ3070
(TmFAE) DNA. No detectable Erucic (22:1) acid peaks
were reported for these transgenic lines.

| Sample ID | C18:1 | C18:2 | C18:3 a | C20:1 | C22:2n6 | C22:5 |
|---|---|---|---|---|---|---|
| STRAIN Z; | 59.97 | 7.44 | 0.56 | 0.57 | 0.00 | 0.00 |
| T588; | | | | | | |
| D1825-47 | | | | | | |
| STRAIN Z; | 58.77 | 7.16 | 0.51 | 0.50 | 0.09 | 0.11 |
| T588; | | | | | | |
| D1825-35 | | | | | | |
| STRAIN Z; | 60.40 | 7.82 | 0.47 | 0.44 | 0.07 | 0.07 |
| T588; | | | | | | |
| D1825-27 | | | | | | |
| STRAIN Z; | 58.07 | 7.32 | 0.54 | 0.41 | 0.05 | 0.05 |
| T588; | | | | | | |
| D1825-14 | | | | | | |
| STRAIN Z; | 58.66 | 7.74 | 0.46 | 0.39 | 0.08 | 0.00 |
| T588; | | | | | | |
| D1825-40 | | | | | | |
| Cntrl | 57.99 | 6.62 | 0.56 | 0.19 | 0.05 | 0.05 |
| STRAIN Z | | | | | | |
| pH 7 | | | | | | |
| Cntrl | 57.70 | 7.08 | 0.54 | 0.11 | 0.06 | 0.05 |
| STRAIN Z | | | | | | |
| pH 5 | | | | | | |

TABLE 16
Unsaturated fatty acid profile in STRAIN Z and representative
derivative transgenic lines transformed with pSZ3068
(BnFAE1) DNA. No detectable Erucic (22:1) acid peaks
were reported for these transgenic lines.

| Sample ID | C18:1 | C18:2 | C18:3 a | C20:1 | C22:2n6 | C22:5 |
|---|---|---|---|---|---|---|
| STRAIN Z; | 59.82 | 7.88 | 0.55 | 0.32 | 0.17 | 0.10 |
| T588; | | | | | | |
| D1826-30 | | | | | | |
| STRAIN Z; | 59.32 | 8.02 | 0.58 | 0.27 | 0.18 | 0.07 |
| T588; | | | | | | |
| D1826-23 | | | | | | |
| STRAIN Z; | 59.63 | 7.49 | 0.55 | 0.27 | 0.19 | 0.08 |
| T588; | | | | | | |
| D1826-45 | | | | | | |
| STRAIN Z; | 59.35 | 7.78 | 0.57 | 0.26 | 0.23 | 0.00 |
| T588; | | | | | | |
| D1826-24 | | | | | | |
| STRAIN Z; | 59.14 | 7.61 | 0.57 | 0.25 | 0.22 | 0.05 |
| T588; | | | | | | |
| D1826-34 | | | | | | |
| Cntrl | 57.81 | 7.15 | 0.59 | 0.19 | 0.04 | 0.06 |
| STRAIN Z | | | | | | |
| pH 7 | | | | | | |
| Cntrl | 58.23 | 6.70 | 0.58 | 0.18 | 0.05 | 0.06 |
| STRAIN Z | | | | | | |
| pH 5 | | | | | | |

TABLE 17
Unsaturated fatty acid profile in STRAIN Z and representative
derivative transgenic lines transformed with pSZ3069
(BnFAE2) DNA. No detectable Erucic (22:1) acid peaks
were reported for these transgenic lines.

| Sample ID | C18:1 | C18:2 | C18:3 a | C20:1 | C22:2n6 | C22:5 |
|---|---|---|---|---|---|---|
| STRAIN Z; | 60.59 | 8.20 | 0.57 | 0.34 | 0.00 | 0.07 |
| T588; | | | | | | |
| D1827-6 | | | | | | |
| STRAIN Z; | 59.62 | 6.44 | 0.52 | 0.30 | 0.07 | 0.00 |
| T588; | | | | | | |
| D1827-42 | | | | | | |
| STRAIN Z; | 59.71 | 7.99 | 0.59 | 0.30 | 0.06 | 0.00 |
| T588; | | | | | | |
| D1827-48 | | | | | | |
| STRAIN Z; | 60.66 | 8.21 | 0.59 | 0.29 | 0.04 | 0.00 |
| T588; | | | | | | |
| D1827-43 | | | | | | |
| STRAIN Z; | 60.26 | 7.99 | 0.57 | 0.28 | 0.04 | 0.00 |
| T588; | | | | | | |
| D1827-3 | | | | | | |
| Cntrl | 57.81 | 7.15 | 0.59 | 0.19 | 0.04 | 0.06 |
| STRAIN Z | | | | | | |
| pH 7 | | | | | | |
| Cntrl | 58.23 | 6.70 | 0.58 | 0.18 | 0.05 | 0.06 |
| STRAIN Z | | | | | | |
| pH 5 | | | | | | |

### Example 6

### Tag Regiospecificity in UTEX1435 by Expression of Cuphea PSR23 LPAAT2 and LPAAT3 Genes

We have demonstrated that the expression of 2 different 1-acyl-sn-glycerol-3-phosphate acyltransferases (LPAATs), the LPAAT2 and LPAAT3 genes from Cuphea PSR23 (CuPSR23) in the UTEX1435 derivative strain S2014 resulted in elevation of C10:0, C12:0 and C14:0 fatty acids levels. In this example we provide evidence that Cuphea PSR23 LPAAT2 exhibits high specificity towards incorporating C10:0 fatty acids at sn-2 position in TAGs. The Cuphea PSR23 LPAAT3 specifically incorporates C18:2 fatty acids at sn-2 position in TAGs.

Composition and properties of Prototheca moriformis (UTEX 1435) transgenic strain B, transforming vectors pSZ2299 and pSZ2300 that express CuPSR23 LPAAT2 and LPAAT3 genes, respectively, and their sequences were described previously.

To determine the impact of Cuphea PSR23 LPAAT genes on the resulting fatty acid profiles we have taken advantage of Strain B which synthesizes both mid chain and long chain fatty acids at relatively high levels. As shown in Table 18, the expression of the LPAAT2 gene (D1520) in Strain B resulted in increased C10-C12:0 levels (up to 12% in the best strain, D1520.3-7) suggesting that this LPAAT is specific for mid chain fatty acids. Alternatively, expression of the LPAAT3 gene resulted in a relatively modest increase, (up to 5% in the best strain, D1521.28-7) indicating it has little or no impact on mid-chain levels.

TABLE 18
Fatty acid profiles of Strain B and representative transgenic
lines transformed with pSZ2299 (D1520) and pSZ2300 (D1521) DNA.

| | Fatty Acid (area %) | | | | | | | | | Total |
|---|---|---|---|---|---|---|---|---|---|---|
| Strain | C8:0 | C10:0 | C12:0 | C14:0 | C16:0 | C18:0 | C18:1 | C18:2 | C10-C12 | Saturates |
| Strain B | 0.09 | 4.95 | 29.02 | 15.59 | 12.55 | 1.27 | 27.93 | 7.60 | 33.97 | 63.47 |
| D1520.8-6 | 0.00 | 6.71 | 31.15 | 15.80 | 13.04 | 1.42 | 24.32 | 6.56 | 37.86 | 68.12 |
| D1520.13-4 | 0.00 | 6.58 | 30.96 | 16.14 | 13.34 | 1.25 | 24.32 | 6.27 | 37.54 | 68.27 |
| D1520.19-4 | 0.00 | 7.53 | 32.94 | 16.64 | 12.63 | 1.17 | 21.96 | 6.11 | 40.47 | 70.91 |
| D1520.3-7 | 0.06 | 9.44 | 36.26 | 16.71 | 11.44 | 1.28 | 18.41 | 5.59 | 45.70 | 75.19 |
| D1521.13-8 | 0.00 | 6.21 | 33.13 | 16.70 | 12.30 | 1.18 | 20.84 | 8.70 | 39.34 | 69.52 |
| D1521.18-2 | 0.00 | 5.87 | 31.91 | 16.46 | 12.60 | 1.22 | 22.14 | 8.59 | 37.78 | 68.06 |
| D1521.24-8 | 0.00 | 5.75 | 31.47 | 16.13 | 12.60 | 1.42 | 23.31 | 8.22 | 37.22 | 67.37 |
| D1521.28-7 | 0.00 | 6.28 | 32.82 | 16.33 | 12.27 | 1.43 | 21.98 | 7.91 | 39.10 | 69.13 |

To determine if expression of the Cuphea PSR23 LPAAT genes affected regiospecificity of fatty acids at the sn-2 position, we analyzed TAGs from representative D1520 and D1521 strains utilizing the porcine pancreatic lipase method. As demonstrated in Table 19, the Cuphea PSR23 LPAAT2 gene shows remarkable specificity towards C10:0 fatty acids and appears to incorporate 50% more C10:0 fatty acids into the sn-2 position. The Cuphea PSR23 LPAAT3 gene appears to act exclusively on C18:2 fatty acids, resulting in redistribution of C18:2 fatty acids onto sn-2 position. Accordingly, microbial triglyceride oils with sn-2 profiles of greater than 15% or 20% C10:0 or C18:2 fatty acids are obtainable by introduction of an exogenous LPAAT gene having corresponding specificity.

TABLE 19
TAG and sn-2 fatty acid profiles in oils of parental
S2014 strain and the progeny strains expressing Cuphea
PSR23 LPAAT2 (BJ) and LPAAT3 (BK) genes.

| | | Strain | | | | | |
|---|---|---|---|---|---|---|---|
| | | Strain | | Strain BI | | Strain BK | |
| | | B | | (D1520.3-7) | | (D1521.13-8) | |
| | | Analysis | | | | | |
| | | TAG | sn-2 | TAG | sn-2 | TAG | sn-2 |
| | | Profile | Profile | Profile | Profile | Profile | Profile |
| Fatty | C8:0 | 0 | 0 | 0.1 | 0 | 0 | 0 |
| Acid | C10:0 | 12 | 14.2 | 11 | 24.9 | 6.21 | 6.3 |
| (area | C12:0 | 42.8 | 25.1 | 40.5 | 24.3 | 33.13 | 19.5 |
| %) | C14:0 | 12.1 | 10.4 | 16.3 | 10 | 16.7 | 11.8 |
| | C16:0 | 7.3 | 1.3 | 10.2 | 1.4 | 12.3 | 3 |
| | C18:0 | 0.7 | 0.2 | 0.9 | 0.6 | 1.18 | 0.5 |
| | C18:1 | 18.5 | 36.8 | 15.4 | 29.2 | 20.84 | 36.3 |
| | C18:2 | 5.8 | 10.9 | 4.9 | 8.7 | 8.7 | 20.9 |
| | C18:3a | 0.6 | 0.8 | 0.4 | 0.8 | 0.48 | 1.2 |
| | C10- | 66.9 | 49.7 | 67.8 | 59.2 | 56.0 | 37.6 |
| | C14 | | | | | | |
| | C10- | 54.8 | 39.3 | 51.5 | 49.2 | 39.3 | 25.8 |
| | C12 | | | | | | |

### Example 7

### A Suite of Regulatable Promoters to Conditionally Control Gene Expression Levels in Oleaginous Cells in Synchrony with Lipid Production

S5204 was generated by knocking out both copies of FATAL in Prototheca moriformis (PmFATA1) while simultaneously overexpressing the endogenous PmKASII gene in a Δfad2 line, S2532. S2532 itself is a FAD2 (also known as FADc) double knockout strain that was previously generated by insertion of C. tinctorius ACP thioesterase (Accession No: AAA33019.1) into S1331, under the control of CrTUB2 promoter at the FAD2 locus. S5204 and its parent S2532 have a disrupted endogenous PmFAD2-1 gene resulting in no 412 specific desaturase activity manifested as 0% C18:2 (linoleic acid) levels in both seed and lipid production stages. Lack of any C18:2 in S5204 (and its parent S2532) results in growth defects which can be partially mitigated by exogenous addition of linoleic acid in the seed stage. For industrial applications of a zero linoleic oil however, exogenous addition of linoleic acid entails additional cost. We have previously shown that complementation of S5204 (and other Δfad2 strains S2530 and S2532) with pH inducible AMT03p driven PmFAD2-1 restores C18:2 to wild-type levels at pH 7.0 and also results in rescued growth characteristics during seed stage without any linoleic supplementation. Additionally when the seed from pH 7.0 grown complemented lines is subsequently transferred into low-nitrogen lipid production flasks with pH adjusted to 5.0 (to control AMT03p driven FAD2 protein levels), the resulting final oil profile matches the parent S5204 or S2532 profile with zero linoleic levels but with rescued growth and productivity metrics. Thus in essence with AMT03p driven FAD2-1 we have developed a pH regulatable strain that potentially could be used to generate oils with varying linoleic levels depending on the desired application.

Prototheca moriformis undergoes rapid cell division during the first 24-30 hrs in fermenters before nitrogen runs out in the media and the cells switch to storing lipids. This initial cell division and growth in fermenters is critical for the overall strain productivity and, as reported above, FAD2 protein is crucial for sustaining vigorous growth characteristic of a particular strain. However when first generation, single insertion, genetically clean, PmFAD2-1 complemented strains (S4694 and S4695) were run in 7 L fermenters at pH 5.0 (with seed grown at pH 7.0), they did not perform on par with the original parent base strain (S1331) in terms of productivity. Western data suggested that AMT03p promoter driving PmFAD2-1 (as measured by FAD2 protein levels) is severely down regulated between 0-30 hrs in fermenters irrespective of fermenter pH (5.0 or 7.0). Work on fermentation conditions (batched vs unbatched/limited initial N, pH shift from 7 to 5 at different time points during production phase) suggested that initial batching (and excess amounts) of nitrogen during early lipid production was the likely cause of AMT03p promoter down regulation in fermenters. Indeed, this initial repression in AMT03 can be directly seen in transcript time-course during fermentation. A significant depression of Amt03 expression was observed early in the run, which corresponds directly with NH4 levels in the fermenter.

When the fermentations were performed with limited N, we were able to partially rescue the AMT03p promoter activity and while per cell productivity of S4694/S4695 was on par with the parent S1331, the overall productivity still lagged behind. These results suggest that a suboptimal or inactive AMT03p promoter and thus limitation of FAD2 protein in early fermentation stages inhibits any complemented strains from attaining their full growth potential and overall productivity. Here we identify new, improved promoter that allow differential gene activity during high-nitrogen growth and low-nitrogen lipid production phases.

In particular, we observed that:
In trans expression of the fatty acid desaturase-2 gene from Prototheca moriformis (PmFad2-1) under the control of down regulated promoter elements identified using a transcriptome based bioinformatics approach results in functional complementation of PmFAD2-1 with restored growth in Δfad2, Δfata1 strain S5204.
Complementation of S5204 manifested in a robust growth phenotype only occurs in seed and early fermentation stages when the new promoter elements are actively driving the expression of PmFAD2-1.
Once the cells enter the active lipid production phase (around the time when N runs out in the fermenter), the newly identified promoters are down regulated resulting in no additional FAD2 protein and the final oil profile of the complemented lines is same as the parent S5204 albeit with better growth characteristics.
These strains should potentially mitigate the problems that were encountered with AMT03p driven FAD2 in earlier complemented strains.
Importantly, we have identified down-regulatable promoters of varying strengths, some of which are relatively strong in the beginning with low-to-moderate levels provided during the remainder of the run. Thus depending on phenotype these promoters can be selected for fine-tuning the desired levels of transgenes.

Bioinformatics Methods:

RNA was prepared from cells taken from 8 time points during a typical fermenter run. RNA was polyA-selected for run on an Illumina HiSeq. Illumina paired-end data (100 bp reads×2, ˜600 bp fragment size) was collected and processed for read quality using FastQC [www.bioinformatics.babraham.ac.uk/projects/fastqc/]. Reads were run through a custom read-processing pipeline that de-duplicates, quality-trims, and length-trims reads.

Transcripts were assembled from Illumina paired-end reads using Oases/velvet [Velvet: algorithms for de novo short read assembly using de Bruijn graphs. D. R. Zerbino and E. Birney. Genome Research 18:821-829] and assessed by N50 and other metrics. The transcripts from all 8 time points were further collapsed using CD-Hit. [Limin Fu, Beifang Niu, Zhengwei Zhu, Sitao Wu and Weizhong Li, CD-HIT: accelerated for clustering the next generation sequencing data. Bioinformatics, (2012), 28 (23): 3150-3152. doi: 10.1093/bioinformatics/bts565; Cd-hit: a fast program for clustering and comparing large sets of protein or nucleotide sequences”, Weizhong Li & Adam Godzik Bioinformatics, (2006) 22:1658-9].

These transcripts were used as the base (reference assembly) for expression-level analysis. Reads from the 8 time points were analyzed using RSEM which provides raw read counts as well as a normalized value provided in Transcripts Per Million (TPM). [Li, Bo & Dewey, Colin N. (2011). RSEM: accurate transcript quantification from RNA-Seq data with or without a reference genome, BioMed Central: The Open Access Publisher. Retrieved at Oct. 10, 2012, from the website temoa: Open Educational Resources (OER) Portal at www.temoa.info/node/4416141 The TPM was used to determine expression levels. Genes previously identified in screens for strong promoters were also used to gauge which levels should be considered as significantly high or low. This data was loaded into a Postgres database and visualized with Spotfire, along with integrated data that includes gene function and other characteristics such as categorization based on expression profile. This enabled rapid and targeted analysis of genes with significant changes in expression.

The promoters for genes, which we selected, were mapped onto a high-quality reference genome for 5376 (our reference Prototheca moriformis strain). Briefly, PacBio long reads (˜2 kb) were error-corrected by high-quality PacBio CCS reads (˜600 bp) and assembled using the Allora assembler in SMRTPipe [pacbiodevnet.com]. This reference genome, in conjunction with transcriptome read mapping, was used to annotate the precise gene structures, promoter and UTR locations, and promoter elements within the region of interest, which then guided further sequencing and promoter element selection.

The criteria used for identifying new promoter elements were:
1. Reasonable expression (e.g., >500, <100, or <50 transcripts per million [TPM]) of a downstream gene in seed and early lipid production stages (T0-T30 hrs)
2. Severe down regulation of the gene above (e.g., >5-fold. 10-fold, or 15-fold) when the nitrogen gets depleted in the fermenters.
3. pH neutrality of the promoter elements (e.g., less than a 2-fold change in TPM on going from pH 5.0 top 7.0 in cultivation conditions), or at least effective operation under pH5 conditions.

Using the above described criteria we identified several potentially down regulated promoter elements that were eventually used to drive PmFAD2-1 expression in S5204. A range of promoters was chosen that included some that started as being weak promoters and went down to extremely low levels, through those that started quite high and dropped only to moderately low levels. This was done because it was unclear a priori how much expression would be needed for FAD2 early on to support robust growth, and how little FAD2 would be required during the lipid production phase in order to achieve the zero linoleic phenotype.

The promoter elements that were selected for screening and their allelic forms were named after their downstream gene and are as follows:

1. Carbamoyl phosphate synthase (PmCPS1p and PmCPS2p)

2. Dipthine synthase (PmDPS1p and PmDPS2p)

3. Inorganic pyrophosphatase (PmIPP1p)

4. Adenosylhomocysteinase (PmAHC1p and PmAHC2p)

5. Peptidyl-prolyl cis-trans isomerase (PmPPI1p and PmPPI2p)

6. GMP Synthetase (PmGMPS1p and PmGMPS2p)

7. Glutamate Synthase (PmGSp)

8. Citrate Synthase (PmCS1p and PmCS2p)

9. Gamma Glutamyl Hydrolase (PmGGH1p)

10. Acetohydroxyacid Isomerase (PmAHI1p and PmAHI2p)

11. Cysteine Endopeptidase (PmCEP1p)

12. Fatty acid desaturase 2 (PmFAD2-1p and PmFad2-2p) [CONTROL]

The transcript profile of two representative genes viz. PmIPP (Inorganic Pyrophosphatase) and PmAHC, (Adenosylhomocysteinase) start off very strong (4000-5000 TPM) but once the cells enter active lipid production their levels fall off very quickly. While the transcript levels of PmIPP drop off to nearly 0 TPM, the levels of PmAHC drop to around 250 TPM and then stay steady for the rest of the fermentation. All the other promoters (based on their downstream gene transcript levels) showed similar downward expression profiles.

The elements were PCR amplified and wherever possible promoters from allelic genes were identified, cloned and named accordingly e.g. the promoter elements for 2 genes of Carbamoyl phosphate synthase were named PmCPS1p and PmCPS2p. As a comparator promoter elements from PmFAD2-1 and PmFAD2-2 were also amplified and used to drive PmFAD2-1 gene. While, in the present example, we used FAD2-1 expression and hence C18:2 levels to interrogate the newly identified down regulated promoters, in principle these promoter elements can be used to down regulate any gene of interest.

Construct Used for the Expression of the Prototheca moriformis Fatty Acid Desaturase 2 (PmFAD2-1) Under the Expression of PmCPS1p in Δfad2 Strains S5204—[pSZ3377]:

The Δfad2 Δfata1 S5204 strain was transformed with the construct pSZ3377. The sequence of the transforming DNA is provided below. Relevant restriction sites in the construct pSZ3377 (6S::PmHXT1p-ScMEL1-CvNR::PmCPS1p-PmFAD2-1-CvNR::6S) are indicated in lowercase, underlined and bold, and are from 5′-3′ BspQ 1, KpnI, SpeI, SnaBI, EcoRV, SpeI, AflII, SacI, BspQ I, respectively. BspQI sites delimit the 5′ and 3′ ends of the transforming DNA. Bold, lowercase sequences represent genomic DNA from UTEX 1435 that permits targeted integration of the transforming DNA at the 6S locus via homologous recombination. Proceeding in the 5′ to 3′ direction, the Hexose transporter (HXT1) gene promoter from UTEX 1435 driving the expression of the Saccharomyces cerevisiae Melibiase (ScMEL1) gene is indicated by the boxed text. The initiator ATG and terminator TGA for ScMEL1 are indicated by uppercase, bold italics while the coding region is indicated in lowercase italics. The Chlorella vulgaris nitrate reductase 3′ UTR is indicated by lowercase underlined text followed by an UTEX 1435 CPS1p promoter of Prototheca moriformis, indicated by boxed italics text. The Initiator ATG and terminator TGA codons of the PmFAD2-1 are indicated by uppercase, bold italics, while the remainder of the gene is indicated by bold italics. The C. vulgaris nitrate reductase 3′ UTR is again indicated by lowercase underlined text followed by the UTEX 1435 6S genomic region indicated by bold, lowercase text. The final construct was sequenced to ensure correct reading frames and targeting sequences.

Nucleotide sequence of transforming DNA contained in plasmid pSZ3377:
(SEQ ID NO: 41)
gctcttcggagtcactgtgccactgagttcgactggtagctgaatggagtcgctgctccactaaacgaattgtcagcaccgcca
gccggccgaggacccgagtcatagcgagggtagtagcgcgccatggcaccgaccagcctgcttgccagtactggcgtctcttc
cgcttctctgtggtcctctgcgcgctccagcgcgtgcgcttttccggtggatcatgcggtccgtggcgcaccgcagcggccgctg
cccatgcagcgccgctgcttccgaacagtggcggtcagggccgcacccgcggtagccgtccgtccggaacccgcccaagagt
tttgggagcagcttgagccctgcaagatggcggaggacaagcgcatcttcctggaggagcaccggtgcgtggaggtccgggg
ctgaccggccgtcgcattcaacgtaatcaatcgcatgatgatcagaggacacgaagtcttggtggcggtggccagaaacact
gtccattgcaagggcatagggatgcgttccttcacctctcatttctcatttctgaatccctccctgctcactctttctcctcctccttc

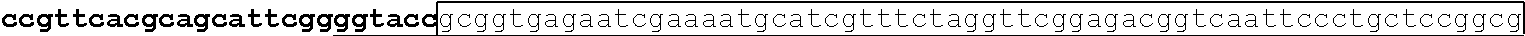

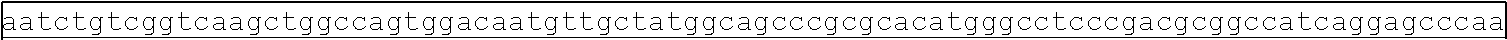

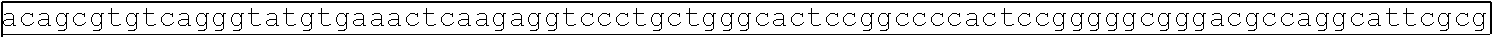

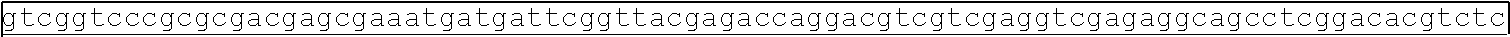

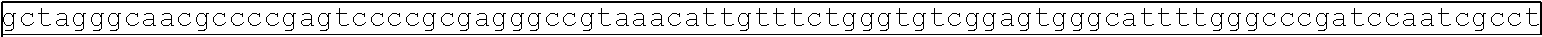

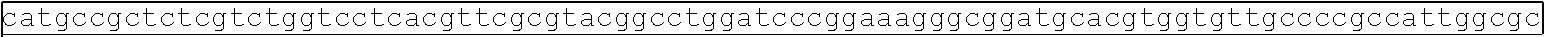

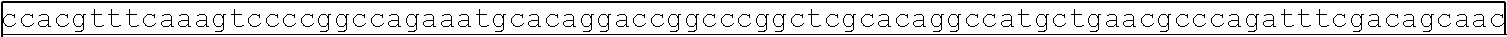

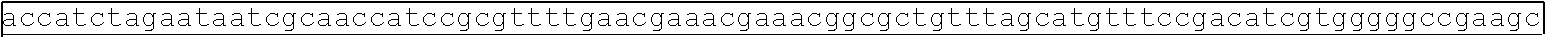

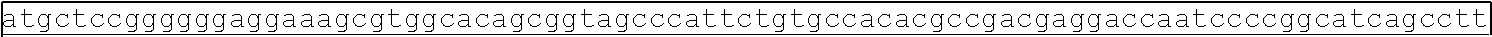

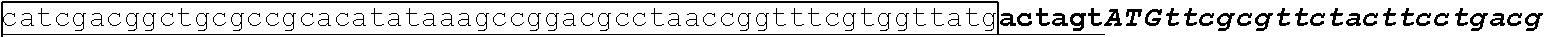

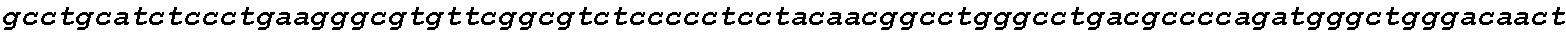

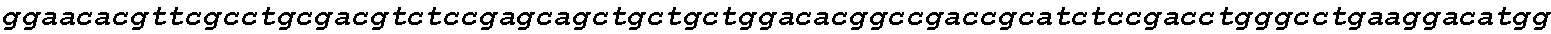

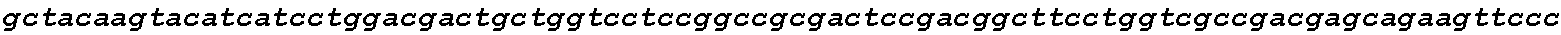

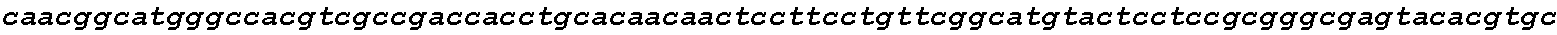

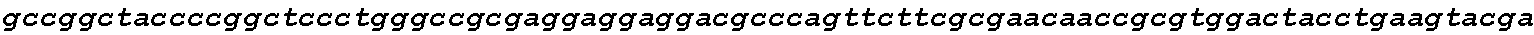

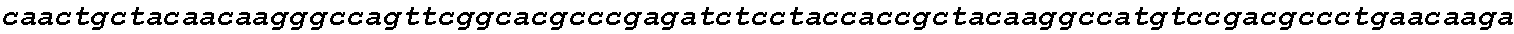

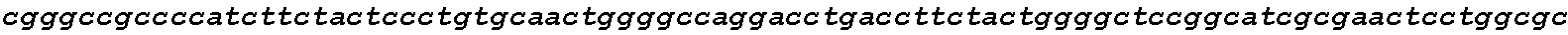

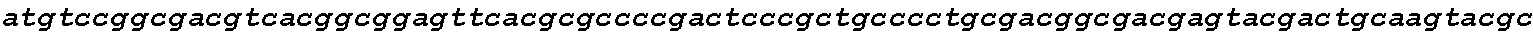

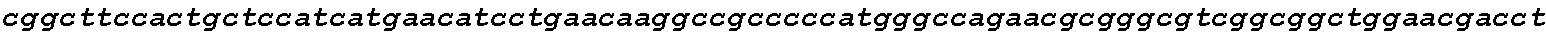

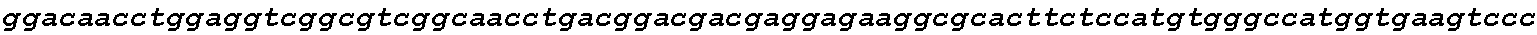

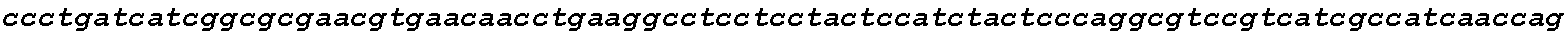

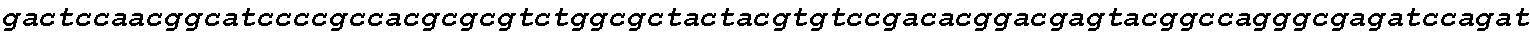

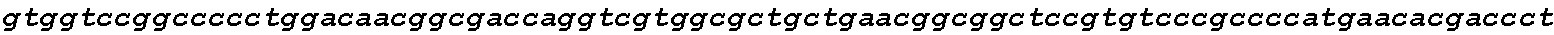

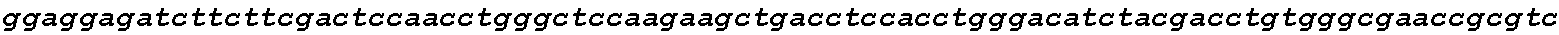

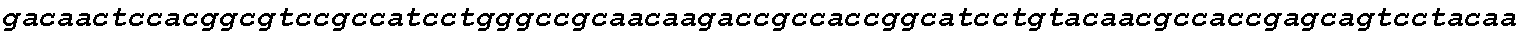

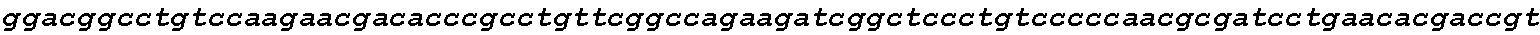

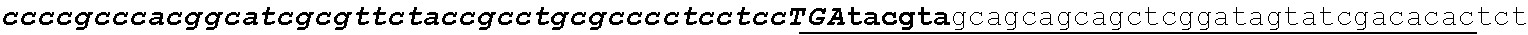

ggacgctggtcgtgtgatggactgttgccgccacacttgctgccttgacctgtgaatatccctgccgcttttatcaaacagcctcagtgtg
tttgatcttgtgtgtacgcgcttttgcgagttgctagctgcttgtgctatttgcgaataccacccccagcatccccttccctcgtttcatatcgc
ttgcatcccaaccgcaacttatctacgctgtcctgctatccctcagcgctgctcctgctcctgctcactgcccctcgcacagccttggtttg
ggctccgcctgtattctcctggtactgcaacctgtaaaccagcactgcaatgctgatgcacgggaagtagtgggatgggaacacaaat

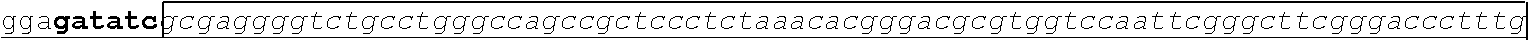

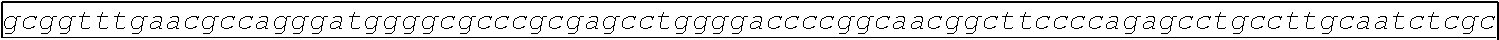

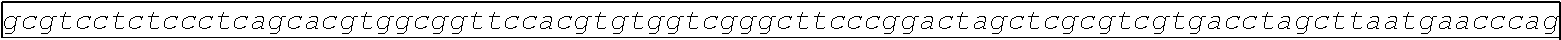

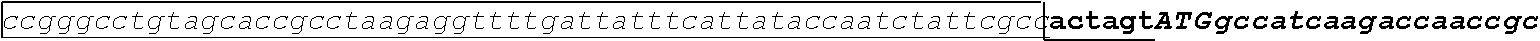

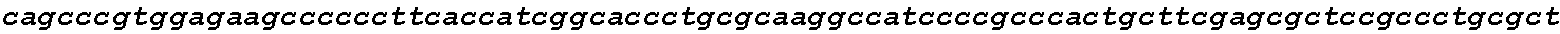

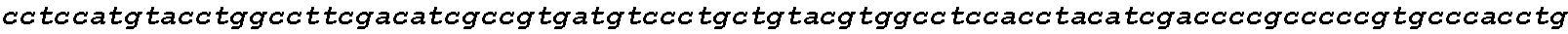

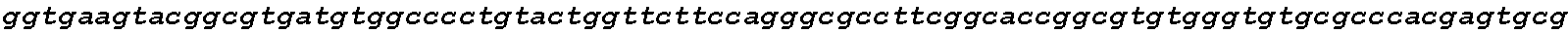

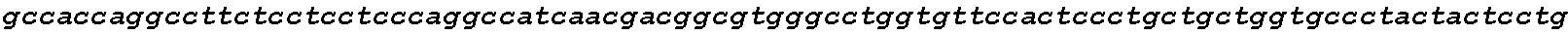

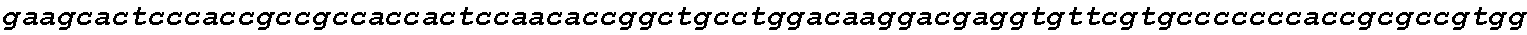

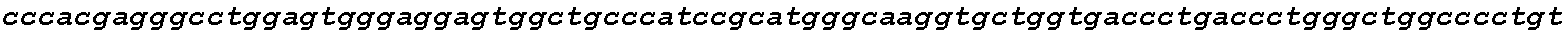

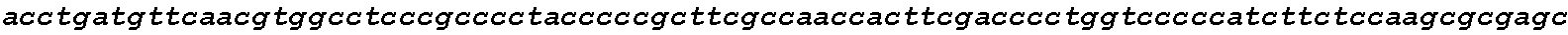

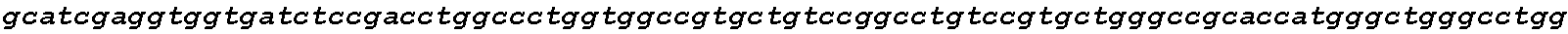

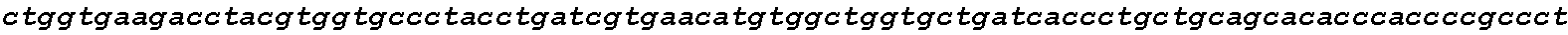

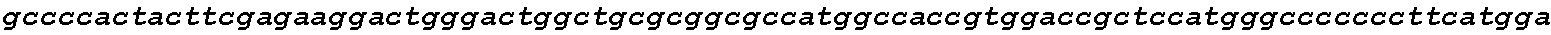

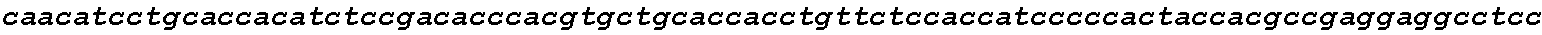

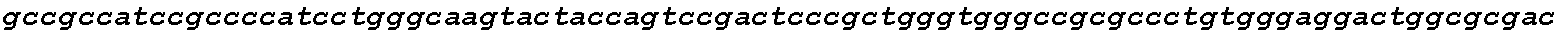

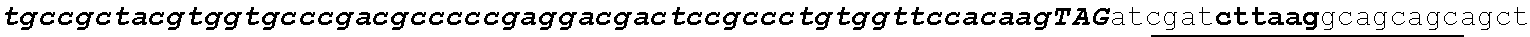

cggatagtatcgacacactctggacgctggtcgtgtgatggactgttgccgccacacttgctgccttgacctgtgaatatccctgccgctt
ttatcaaacagcctcagtgtgtttgatcttgtgtgtacgcgcttttgcgagttgctagctgcttgtgctatttgcgaataccacccccagcatc
cccttccctcgtttcatatcgcttgcatcccaaccgcaacttatctacgctgtcctgctatccctcagcgctgctcctgctcctgctcactgc
ccctcgcacagccttggtttgggctccgcctgtattctcctggtactgcaacctgtaaaccagcactgcaatgctgatgcacgggaagta
gtgggatgggaacacaaatggaaagcttaattaagagctcttgttttccagaaggagttgctccttgagcctttcattctcagcctcg
ataacctccaaagccgctctaattgtggagggggttcgaatttaaaagcttggaatgttggttcgtgcgtctggaacaagccca
gacttgttgctcactgggaaaaggaccatcagctccaaaaaacttgccgctcaaaccgcgtacctctgctttcgcgcaatctgc
cctgttgaaatcgccaccacattcatattgtgacgcttgagcagtctgtaattgcctcagaatgtggaatcatctgccccctgtgc
gagcccatgccaggcatgtcgcgggcgaggacacccgccactcgtacagcagaccattatgctacctcacaatagttcataac
agtgaccatatttctcgaagctccccaacgagcacctccatgctctgagtggccaccccccggccctggtgcttgcggagggca
ggtcaaccggcatggggctaccgaaatccccgaccggatcccaccacccccgcgatgggaagaatctctccccgggatgtgg
gcccaccaccagcacaacctgctggcccaggcgagcgtcaaaccataccacacaaatatccttggcatcggccagaattcct
tctgccgctctgctacccggtgcttctgtccgaagcaggggttgctagggatcgctccgagtccgcaaacccttgtcgcgtggeg
gggcttgttcgagcttgaagagc

The recombination between C. vulgaris nitrate reductase 3′ UTR's in the construct pSZ3377 results in multiple copies of PmFAD2-1 in transgenic lines which would then manifest most likely as higher C18:2 levels at the end of fermentation. Since the goal was to create a strain with 0% terminal C18:2, we took precautions to avoid this recombination. In another version of the above plasmid ScMEL1 gene was followed by Chlorella protothecoides (UTEX 250) elongation factor 1a (CpEF1a) 3′ UTR instead of C. vulgaris 3′ UTR. The sequence of C. protothecoides (UTEX 250) elongation factor 1a (CpEF1a) 3′ UTR used in construct pSZ3384 and other constructs with this 3′ UTR (described below) is shown below. Plasmid pSZ3384 could be written as 6S::PmHXT1p-ScMEL1-CpEF1a::PmCPS1p-PmFAD2-1-CvNR::6S.

Nucleotide sequence of Chlorella protothecoides
(UTEX 250) elongation factor 1a (CpEF1a)
3′ UTR in pSZ3384:
(SEQ ID NO: 42)
tacaacttattacgtaacggagcgtcgtgcgggagggagtgtgccgag
cggggagtcccggtctgtgcgaggcccggcagctgacgctggcgagcc
gtacgccccgagggtccccctcccctgcaccctcttccccttccctct
gacggccgcgcctgttcttgcatgttcagcgacgaggatatc

The C. protothecoides (UTEX 250) elongation factor 1a 3′ UTR sequence is flanked by restriction sites SnaBI on 5′ and EcoRV on 3′ ends shown in lowercase bold underlined text. Note that the plasmids containing CpEF1a 3′ UTR (pSZ3384 and others described below) after ScMEL1 stop codon contains 10 extra nucleotides before the 5′ SnaBI site. These nucleotides are not present in the plasmids that contain C. vulgaris nitrate reductase 3′ UTR after the S. ScMEL1 stop codon.

In addition to plasmids pSZ3377 and pSZ3384 expressing either a recombinative CvNR-Promoter-PmFAD2-1-CvNR or non-recombinative CpEF1a-Promoter-PmFAD2-1-CvNR expression unit described above, plasmids using other promoter elements mentioned above were constructed for expression in S5204. These constructs along with their transformation identifiers (D #) can be described as:

| Plasmid ID | D # | Description |
|---|---|---|
| pSZ3378 | D2090 | 6SA::pPmHXT1-ScarIMEL1-CvNR:PmCPS2p-PmFad2-1-CvNR::6SB |
| pSZ3385 | D2097 | 6SA::pPmHXT1-ScarIMEL1-CpEF1a:PmCPS2p-PmFad2-1-CvNR::6SB |
| pSZ3379 | D2091 | 6SA::pPmHXT1-ScarIMEL1-CvNR:PmDPS1p-PmFad2-1-CvNR::6SB |
| pSZ3386 | D2098 | 6SA::pPmHXT1)-ScarIMEL1-CpEF1a:PmDPS1p-PmFad2-1-CvNR::6SB |
| pSZ3380 | D2092 | 6SA::pPmHXT1-ScarIMEL1-CvNR:PmDPS2p-PmFad2-1-CvNR::6SB |
| pSZ3387 | D2099 | 6SA::pPmHXT1-ScarIMEL1-CpEF1a:PmDPS2p-PmFad2-1-CvNR::6SB |
| pSZ3480 | D2259 | 6SA::pPmHXT1-ScarIMEL1-CvNR:PmIPP1p-PmFad2-1-CvNR::6SB |
| pSZ3481 | D2260 | 6SA::pPmHXT1-ScarIMEL1-CpEF1a:PmIPP1p-PmFad2-1-CvNR::6SB |
| pSZ3509 | D2434 | 6SA::pPmHXT1-ScarIMEL1-CvNR:PmAHC1p-PmFad2-1-CvNR::6SB |
| pSZ3516 | D2266 | 6SA::pPmHXT1-ScarIMEL1-CpEF1a:PmAHC1p-PmFad2-1-CvNR::6SB |
| pSZ3510 | D2435 | 6SA::pPmHXT1-ScarIMEL1-CvNR:PmAHC2p-PmFad2-1-CvNR::6SB |
| pSZ3513 | D2263 | 6SA::pPmHXT1-ScarIMEL1-CvNR:PmPPI1p-PmFad2-1-CvNR::6SB |
| pSZ3689 | D2440 | 6SA::pPmHXT1-ScarIMEL1-CpEF1a:PmPPI1p-PmFad2-1-CvNR::6SB |
| pSZ3514 | D2264 | 6SA::pPmHXT1-ScarIMEL1-CvNR:PmPPI2p-PmFad2-1-CvNR::6SB |
| pSZ3518 | D2268 | 6SA::pPmHXT1-ScarIMEL1-CpEF1a:PmPPI2p-PmFad2-1-CvNR::6SB |
| pSZ3515 | D2265 | 6SA::pPmHXT1-ScarIMEL1-CvNR:PmGMPS1p-PmFad2-1-CvNR::6SB |
| pSZ3519 | D2269 | 6SA::pPmHXT1-ScarIMEL1-CpEF1a:PmGMPS1p-PmFad2-1-CvNR::6SB |
| pSZ3520 | D2270 | 6SA::pPmHXT1-ScarIMEL1-CpEF1a:PmGMPS2p-PmFad2-1-CvNR::6SB |
| pSZ3684 | D2436 | 6SA::pPmHXT1-ScarIMEL1-CvNR:PmCS1p-PmFad2-1-CvNR::6SB |
| pSZ3686 | D2438 | 6SA::pPmHXT1-ScarIMEL1-CpEF1A:PmCS1p-PmFad2-1-CvNR::6SB |
| pSZ3685 | D2437 | 6SA::pPmHXT1-ScarIMEL1-CvNR:PmCS2p-PmFad2-1-CvNR::6SB |
| pSZ3688 | D2439 | 6SA::pPmHXT1-ScarIMEL1-CvNR:PmGGHp-PmFad2-1-CvNR::6SB |
| pSZ3511 | D2261 | 6SA::pPmHXT1-ScarIMEL1-CvNR:PmAHI2p-PmFad2-1-CvNR::6SB |
| pSZ3517 | D2267 | 6SA::pPmHXT1-ScarIMEL1-CpEF1a:PmAHI1p-PmFad2-1-CvNR::6SB |
| pSZ3512 | D2262 | 6SA::pPmHXT1-ScarIMEL1-CvNR:PmCEP1p-PmFad2-1-CvNR::6SB |
| pSZ3375 | D2087 | 6SA::pPmHXT1-ScarIMEL1-CvNR:PmFAD2-1p-PmFad2-1-CvNR::6SB |
| pSZ3382 | D2094 | 6SA::pPmHXT1-ScarIMEL1-CpEF1a:PmFAD2-1p-PmFad2-1-CvNR::6SB |
| pSZ3376 | D2088 | 6SA::pPmHXT1-ScarIMEL1-CvNR:PmFAD2-2p-PmFad2-1-CvNR::6SB |
| pSZ3383 | D2095 | 6SA::pPmHXT1-ScarIMEL1-CpEF1a:PmFAD2-2p-PmFad2-1-CvNR::6SB |

The above constructs are the same as pSZ3377 or pSZ3384 except for the promoter element that drives PmFAD2-1. The sequences of different promoter elements used in the above constructs are shown below.

Nucleotide sequence of Carbamoyl phosphate synthase allele 2 promoter contained
in plasmid pSZ3378 and pSZ3385 (PmCPS2p promoter sequence):
(SEQ ID NO: 43)

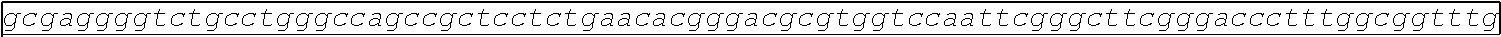

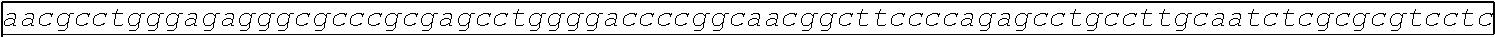

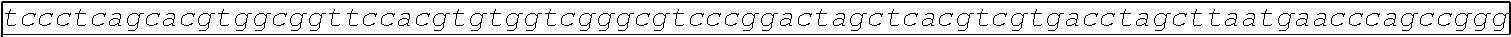

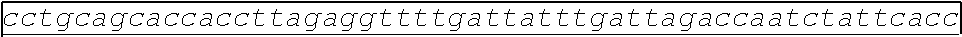

Nucleotide sequence of Dipthine synthase allele 1 promoter contained in plasmid
pSZ3379 and pSZ3386 (PmDPS1p promoter sequence):
(SEQ ID NO: 44)

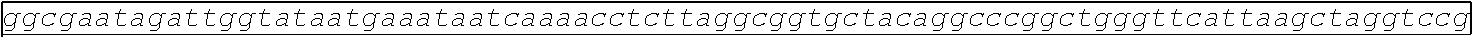

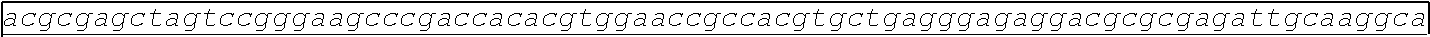

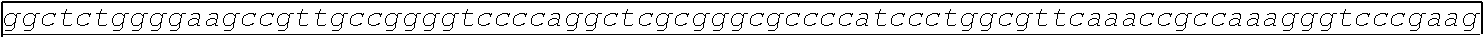

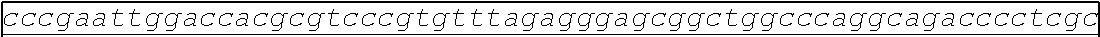

Nucleotide sequence of Dipthine synthase allele 2 promoter contained in plasmid
pSZ3380 and pSZ3387 (PmDPS2p promoter sequence):
(SEQ ID NO: 45)

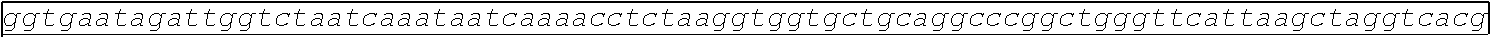

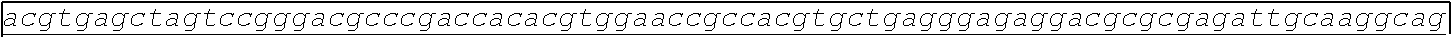

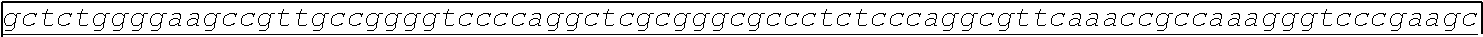

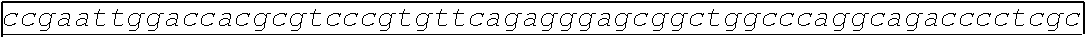

Nucleotide sequence of Inorganic pyrophosphatase allele 1 promoter contained in
plasmid pSZ3480 and pSZ3481 (PmIPP1p promoter sequence):
(SEQ ID NO: 46)

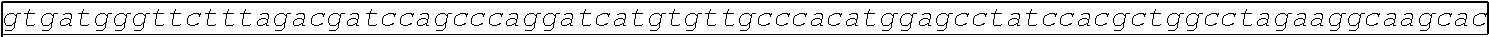

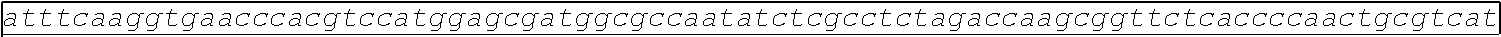

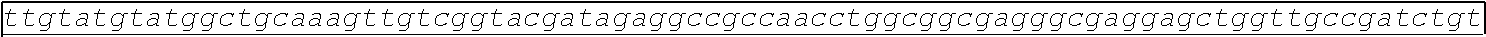

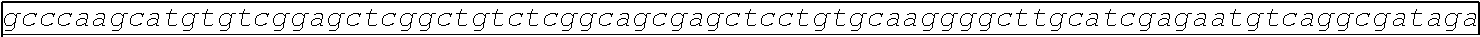

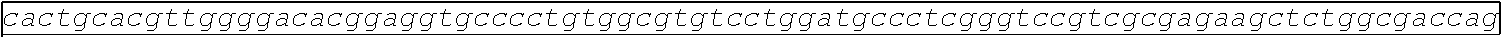

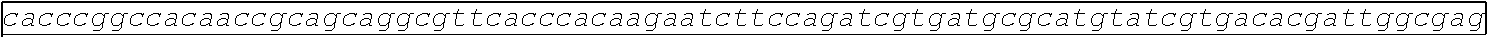

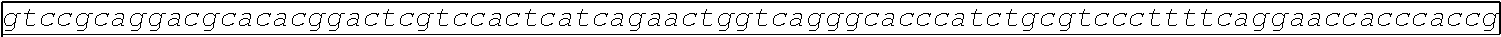

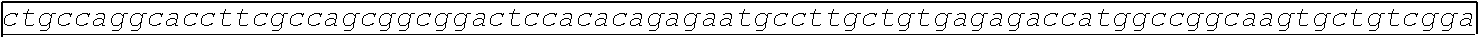

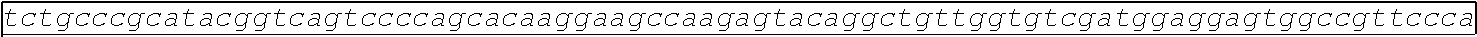

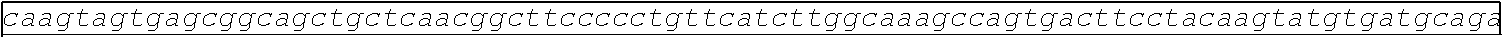

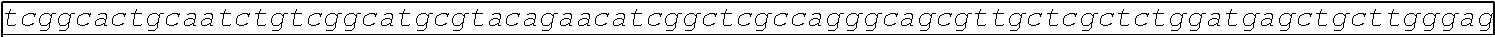

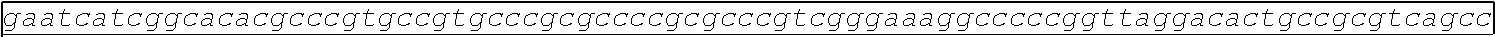

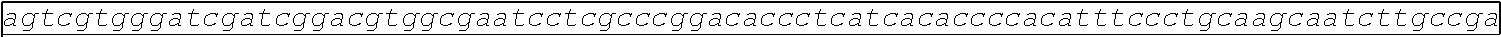

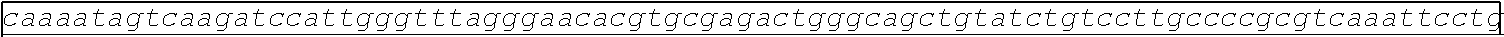

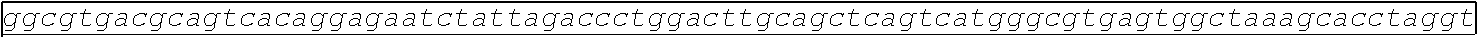

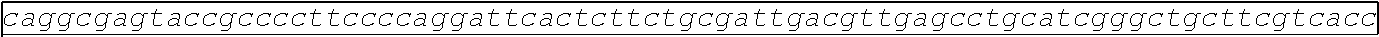

Nucleotide sequence of Adenosylhomocysteinase allele 1 promoter contained in
plasmid pSZ3509 and pSZ3516 (PmAHC1p promoter sequence):
(SEQ ID NO: 47)

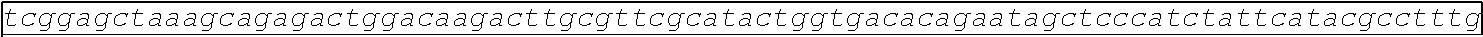

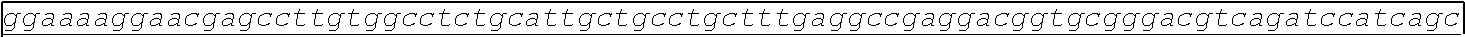

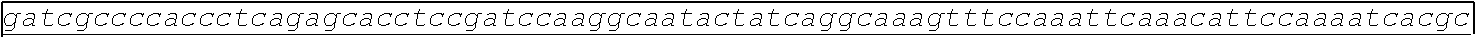

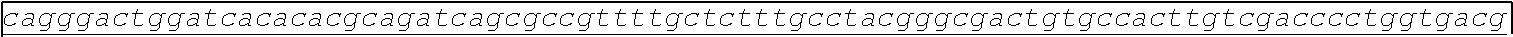

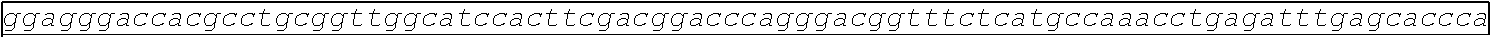

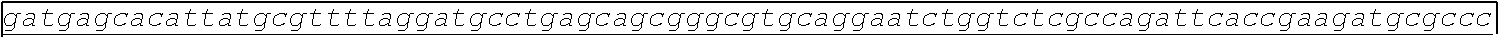

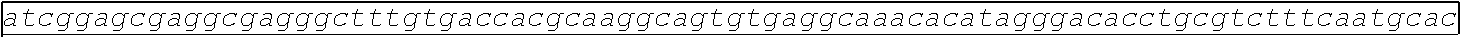

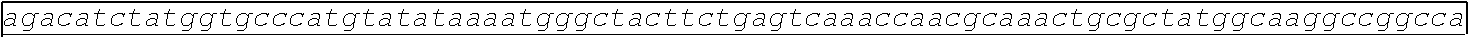

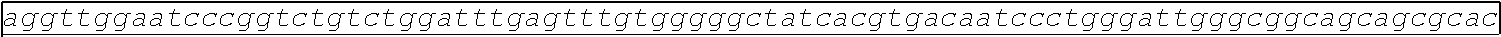

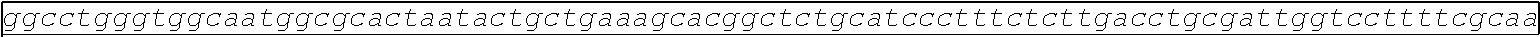

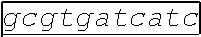

Nucleotide sequence of Adenosylhomocysteinase allele 2 promoter contained in
plasmid pSZ3510 (PmAHC2p promoter sequence):
(SEQ ID NO: 48)

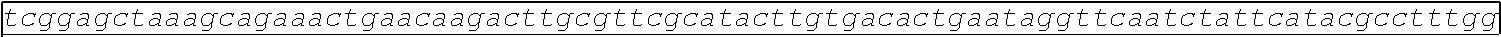

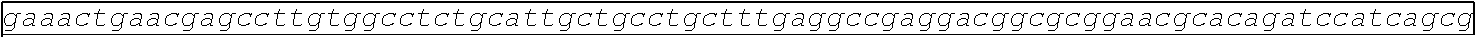

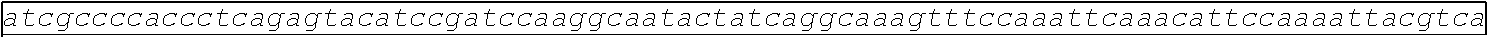

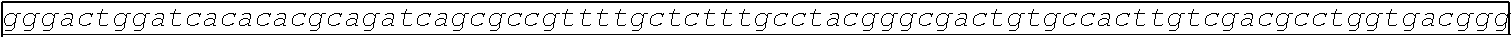

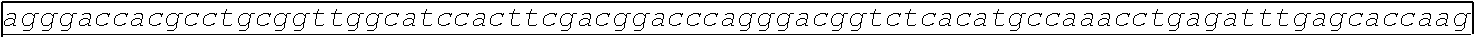

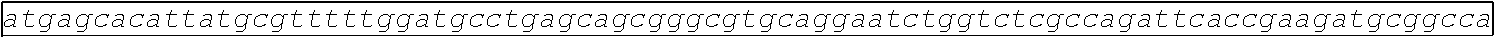

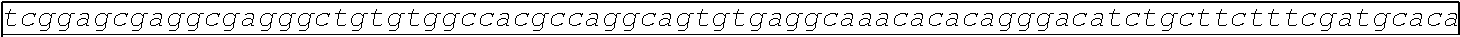

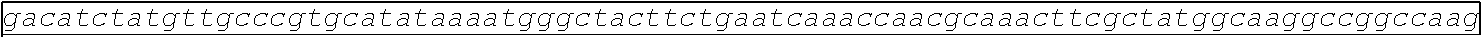

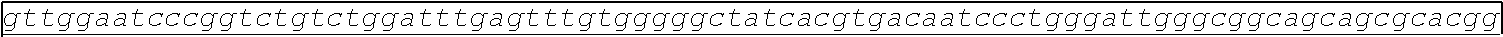

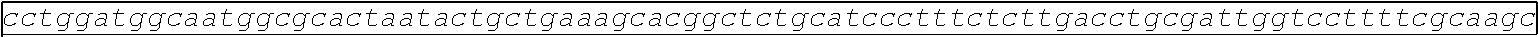

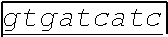

Nucleotide sequence of Peptidyl-prolyl cis-trans isomerase allele 1 promoter
contained in plasmid pSZ3513 and pSZ3689 (PmPPI1p promoter sequence):
(SEQ ID NO: 49)

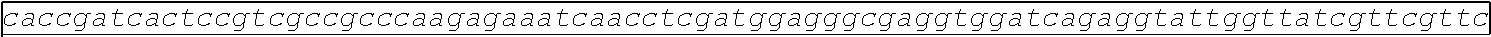

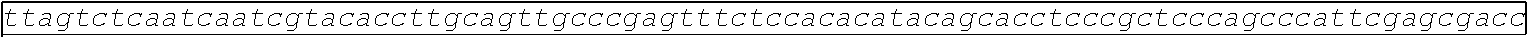

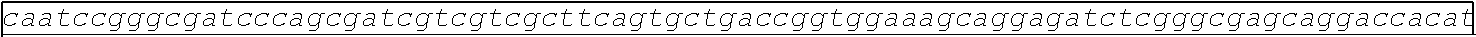

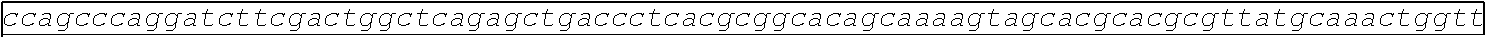

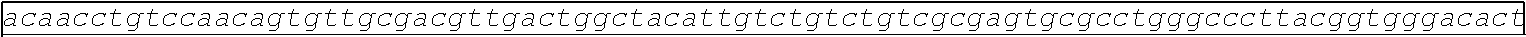

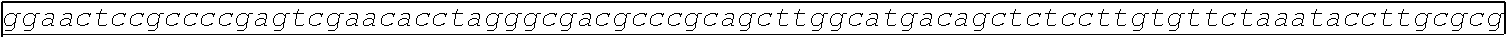

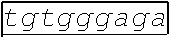

Nucleotide sequence of Peptidyl-prolyl cis-trans isomerase allele 2 promoter
contained in plasmid pSZ3514 and pSZ3518 (PmPPI2p promoter sequence):
(SEQ ID NO: 50)

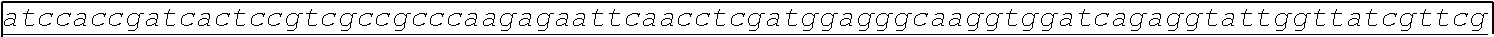

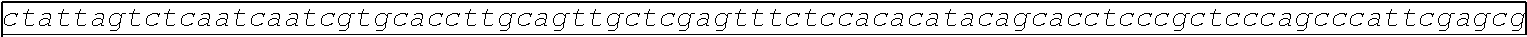

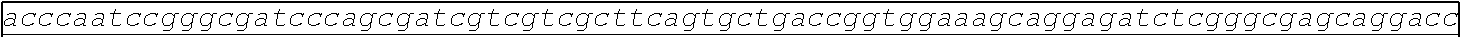

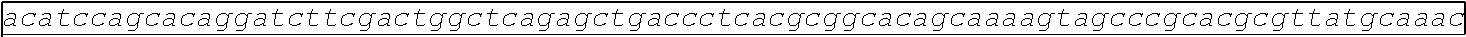

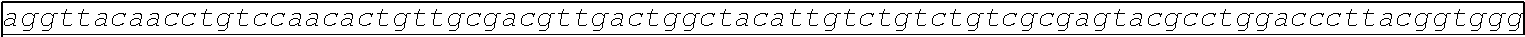

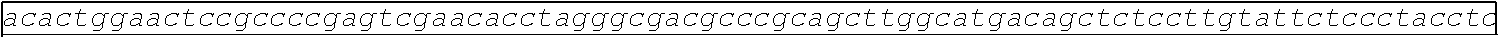

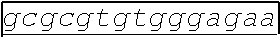

Nucleotide sequence of GMP Synthetase allele 1 promoter contained in plasmid
pSZ3515 and pSZ3519 (PmGMPS 1p promoter sequence):
(SEQ ID NO: 51)

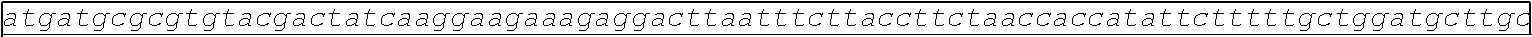

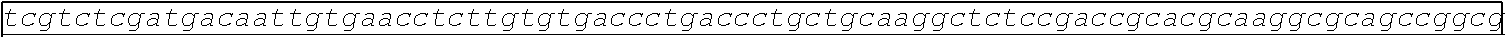

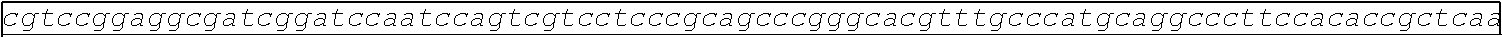

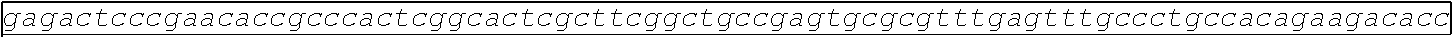

Nucleotide sequence of GMP Synthetase allele 2 promoter contained in plasmid
pSZ3520 (PmGMPS2p promoter sequence):
(SEQ ID NO: 52)

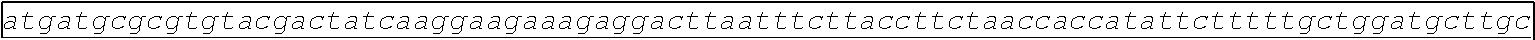

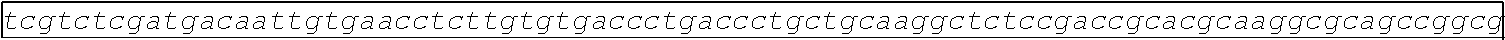

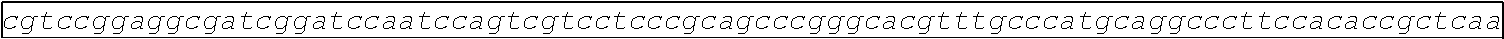

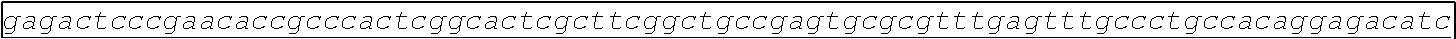

Nucleotide sequence of Citrate synthase allele 1 promoter contained in plasmid
pSZ3684 and pSZ3686 (PmCS1p promoter sequence):
(SEQ ID NO: 53)

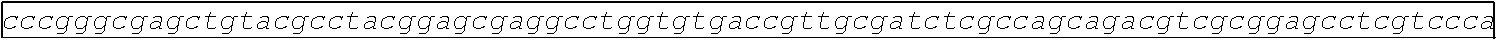

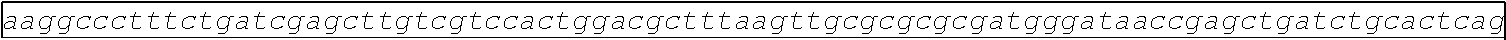

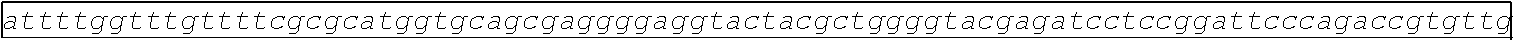

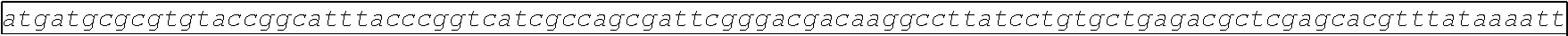

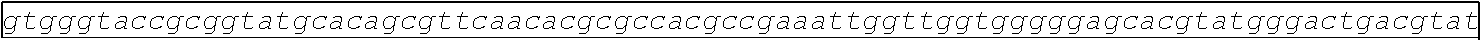

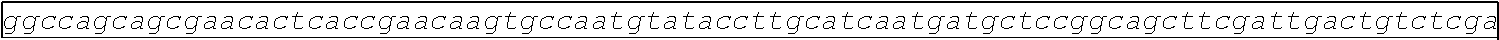

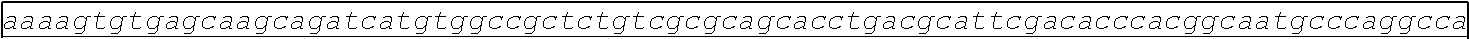

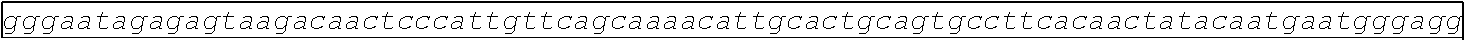

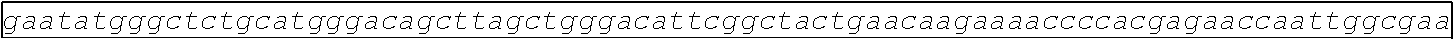

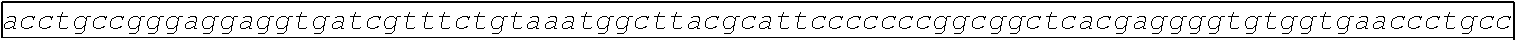

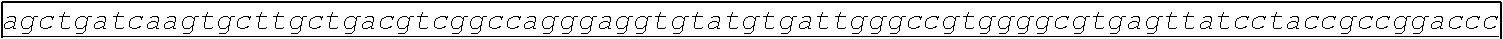

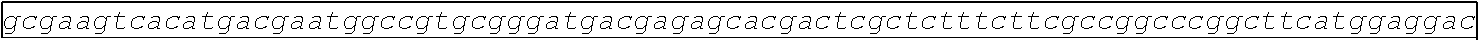

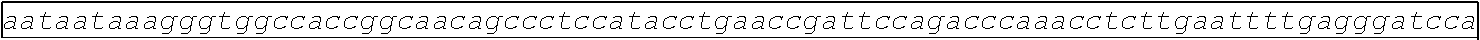

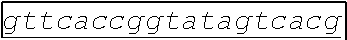

Nucleotide sequence of Citrate synthase allele 2 promoter contained in plasmid
pSZ3685 (PmCS2p promoter sequence):
(SEQ ID NO: 54)

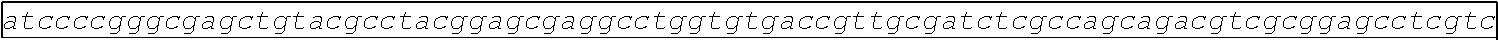

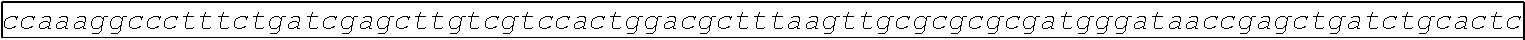

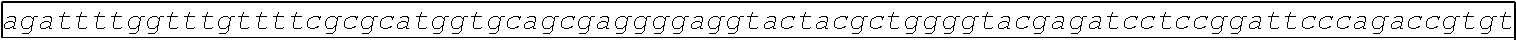

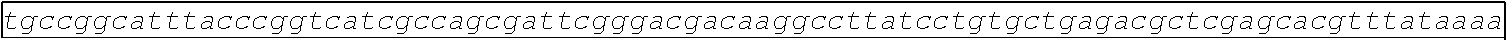

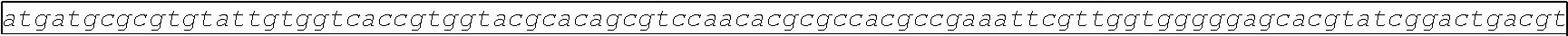

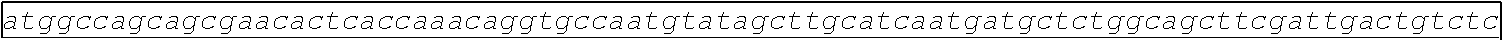

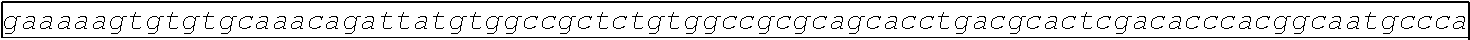

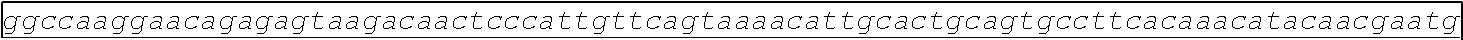

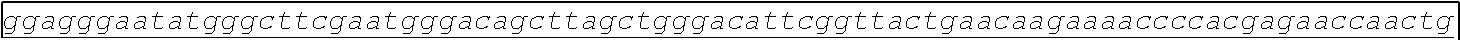

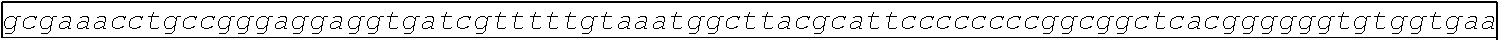

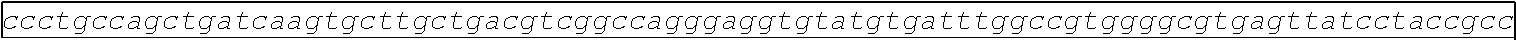

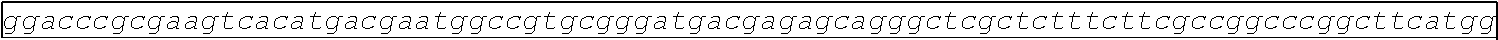

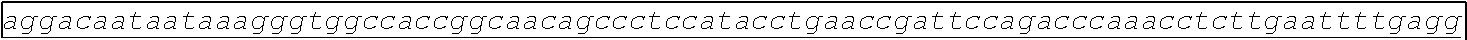

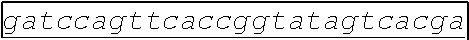

Nucleotide sequence of Gamma Glutamyl Hydrolase allele 1 promoter contained in
plasmid pSZ3688 (PmGGH1p promoter sequence):
(SEQ ID NO: 55)

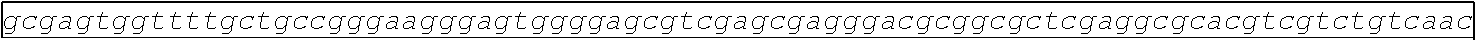

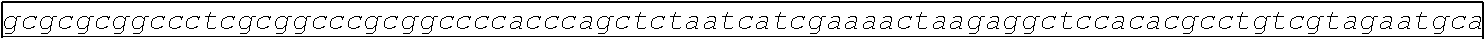

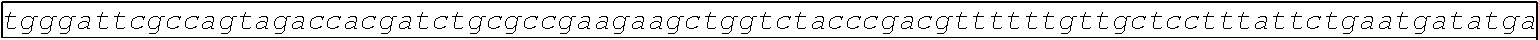

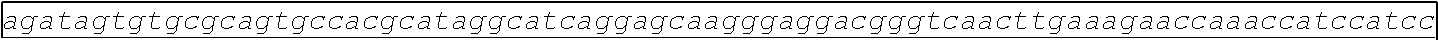

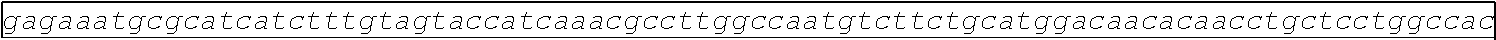

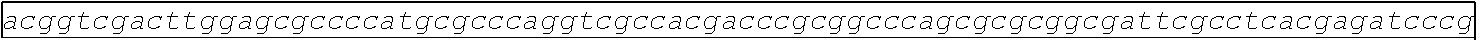

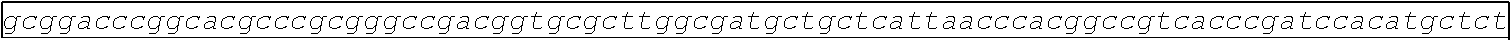

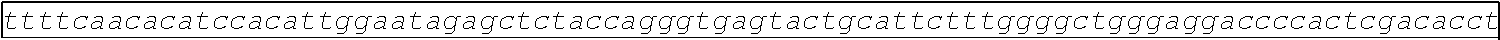

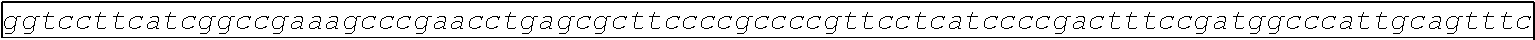

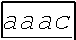

Nucleotide sequence of Acetohydroxyacid Isomerase allele 1 promoter contained in
plasmid pSZ3517 (PmAHI1p promoter sequence):
(SEQ ID NO: 56)

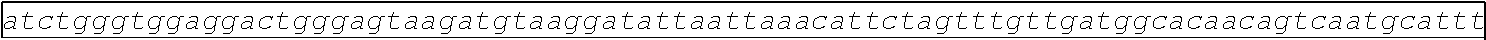

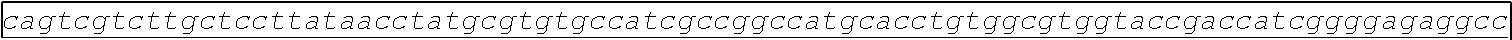

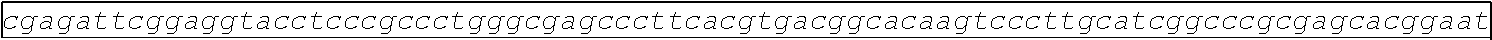

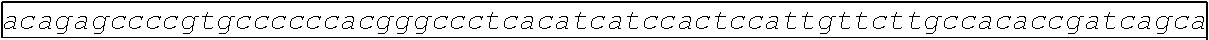

Nucleotide sequence of Acetohydroxyacid Isomerase allele 2 promoter contained in
plasmid pSZ3511 (PmAHI2p promoter sequence):
(SEQ ID NO: 57)

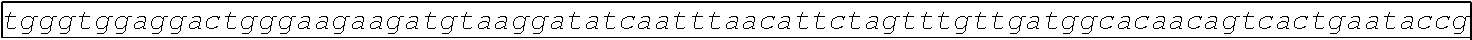

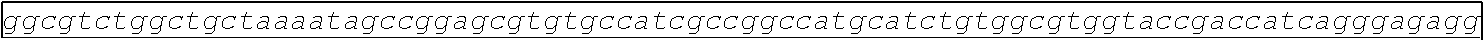

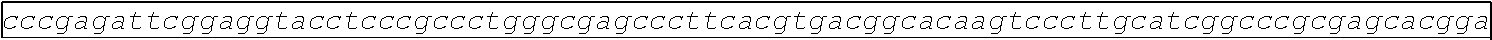

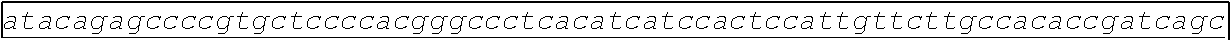

Nucleotide sequence of Cysteine Endopeptidase allele 1 promoter contained in
plasmid pSZ3512 (PmCEP1 promoter sequence):
(SEQ ID NO: 58)

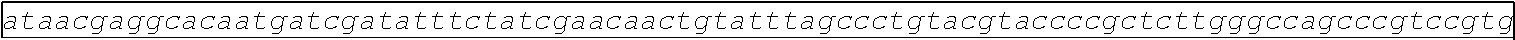

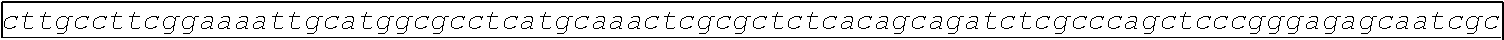

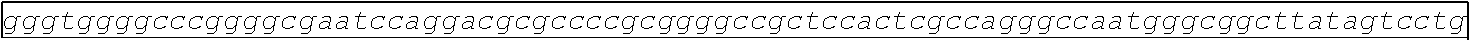

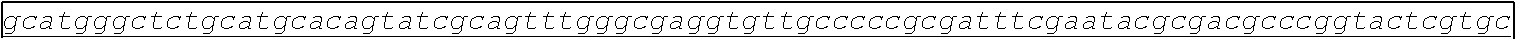

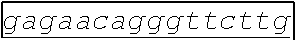

Nucleotide sequence of Fatty acid desaturase 2 allele 1 promoter contained in
plasmid pSZ3375 and 3382 (PmFAD2-1 promoter sequence):
(SEQ ID NO: 59)

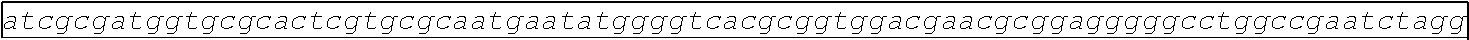

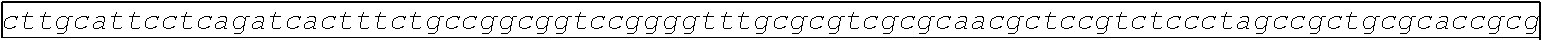

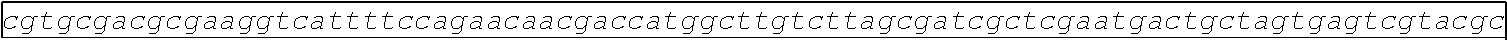

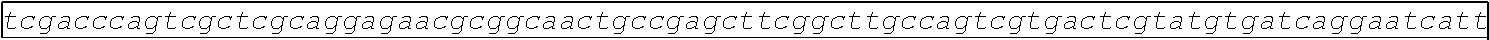

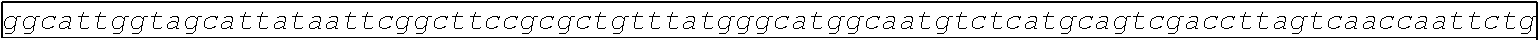

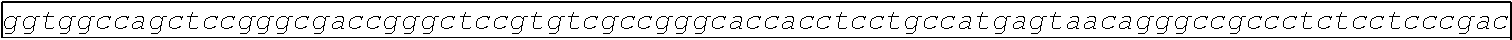

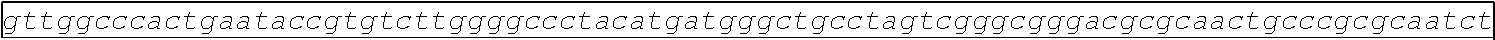

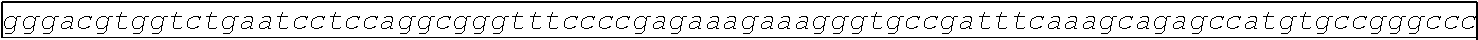

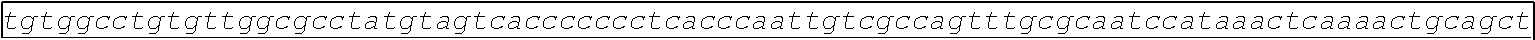

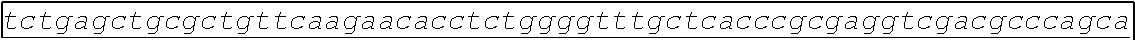

Nucleotide sequence of Fatty acid desaturase 2 allele 2 promoter contained in
plasmid pSZ3376 and 3383 (PmFAD2-2 promoter sequence):
(SEQ ID NO: 60)

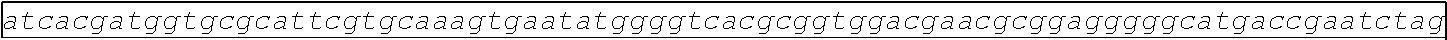

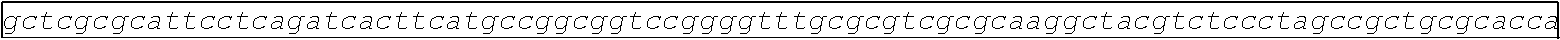

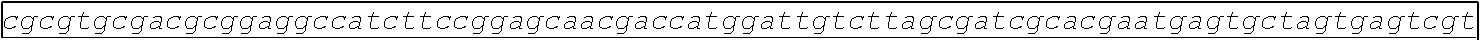

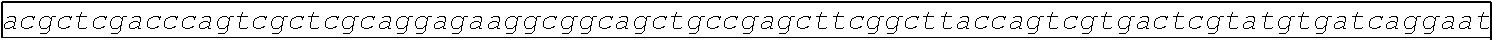

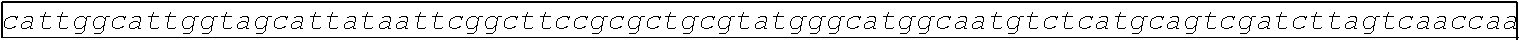

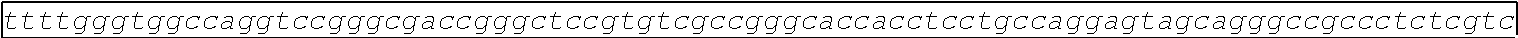

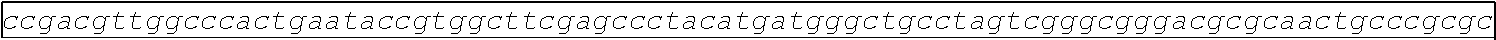

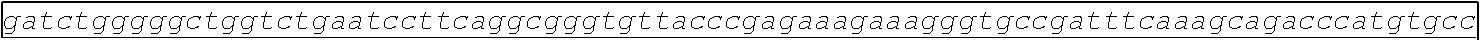

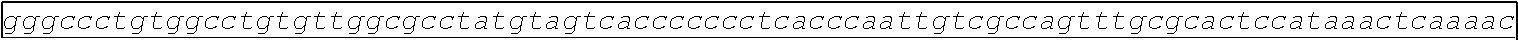

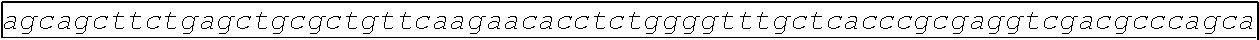

To determine their impact on growth and fatty acid profiles, the above-described constructs were independently transformed into a Δfad2 Δfata1 strain S5204. Primary transformants were clonally purified and grown under standard lipid production conditions at pH5.0 or at pH7.0. The resulting profiles from a set of representative clones arising from transformations are shown in Tables 20-50.

TABLE 20
Fatty acid profile in some representative complemented
(D2087) and parent S5204 lines transformed with pSZ3375
DNA containing PmFAD2-1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 7; S5204; | 0.38 | 4.43 | 1.78 | 83.93 | 7.58 | 0.81 |
| T665; | | | | | | |
| D2087-22 | | | | | | |
| pH 7; S5204; | 0.41 | 4.92 | 1.94 | 83.21 | 7.55 | 0.84 |
| T665; | | | | | | |
| D2087-16 | | | | | | |
| pH 7; S5204; | 0.40 | 4.82 | 1.78 | 83.51 | 7.52 | 0.79 |
| T665; | | | | | | |
| D2087-17 | | | | | | |
| pH 7; S5204; | 1.30 | 8.06 | 2.54 | 79.03 | 7.30 | 0.82 |
| T665; | | | | | | |
| D2087-26 | | | | | | |
| pH 7; S5204; | 1.13 | 7.88 | 2.45 | 79.48 | 7.26 | 0.79 |
| T665; | | | | | | |
| D2087-29 | | | | | | |

TABLE 21
Fatty acid profile in some representative complemented
(D) and parent S5204 lines transformed with pSZ3382
DNA containing PmFAD2-1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 7; S5204; | 0.49 | 5.76 | 2.95 | 83.39 | 5.08 | 0.84 |
| T672; | | | | | | |
| D2094-5 | | | | | | |
| pH 7; S5204; | 0.35 | 5.01 | 2.41 | 85.10 | 5.09 | 0.64 |
| T672; | | | | | | |
| D2094-25 | | | | | | |
| pH 7; S5204; | 0.33 | 5.07 | 2.30 | 84.89 | 5.30 | 0.69 |
| T672; | | | | | | |
| D2094-13 | | | | | | |
| pH 7; S5204; | 0.38 | 4.33 | 1.78 | 85.63 | 5.31 | 0.85 |
| T672; | | | | | | |
| D2094-11 | | | | | | |
| pH 7; S5204; | 0.35 | 5.29 | 2.32 | 84.59 | 5.34 | 0.66 |
| T672; | | | | | | |
| D2094-8 | | | | | | |

TABLE 22
Fatty acid profile in some representative complemented
(D2088) and parent S5204 lines transformed with pSZ3376
DNA containing PmFAD2-2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 7; S5204; | 1.11 | 8.18 | 2.92 | 78.13 | 6.96 | 0.87 |
| T665; | | | | | | |
| D2088-16 | | | | | | |
| pH 7; S5204; | 1.06 | 7.78 | 2.95 | 78.65 | 6.95 | 0.84 |
| T665; | | | | | | |
| D2088-20 | | | | | | |
| pH 7; S5204; | 0.91 | 7.13 | 2.87 | 79.63 | 6.93 | 0.78 |
| T665; | | | | | | |
| D2088-29 | | | | | | |
| pH 7; S5204; | 1.18 | 8.29 | 2.98 | 77.90 | 6.91 | 0.88 |
| T665; | | | | | | |
| D2088-6 | | | | | | |
| pH 7; S5204; | 1.10 | 7.98 | 3.09 | 78.42 | 6.78 | 0.81 |
| T665; | | | | | | |
| D2088-18 | | | | | | |

TABLE 23
Fatty acid profile in some representative complemented
(D) and parent S5204 lines transformed with pSZ3383
DNA containing PmFAD2-2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 7; S5204; | 0.30 | 5.43 | 2.45 | 85.10 | 4.62 | 0.68 |
| T673; | | | | | | |
| D2095-47 | | | | | | |
| pH 7; S5204; | 0.38 | 5.16 | 2.48 | 84.46 | 5.41 | 0.68 |
| T673; | | | | | | |
| D2095-14 | | | | | | |
| pH 7; S5204; | 0.43 | 4.60 | 2.54 | 84.82 | 5.47 | 0.58 |
| T673; | | | | | | |
| D2095-16 | | | | | | |
| pH 7; S5204; | 0.34 | 5.41 | 2.57 | 84.21 | 5.49 | 0.66 |
| T673; | | | | | | |
| D2095-6 | | | | | | |
| pH 7; S5204; | 0.42 | 5.30 | 2.49 | 83.97 | 5.57 | 0.68 |
| T673; | | | | | | |
| D2095-39 | | | | | | |

TABLE 24
Fatty acid profile in representative complemented
(D2089) and parent S5204 lines transformed with pSZ3377
DNA containing PmCPS1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 7; S5204; | 0.35 | 4.73 | 2.29 | 88.94 | 1.79 | 0.39 |
| T672; | | | | | | |
| D2089-40 | | | | | | |
| pH 7; S5204; | 0.51 | 4.85 | 2.96 | 87.55 | 2.05 | 0.41 |
| T672; | | | | | | |
| D2089-2 | | | | | | |
| pH 7; S5204; | 0.56 | 5.00 | 3.04 | 87.24 | 2.07 | 0.36 |
| T672; | | | | | | |
| D2089-14 | | | | | | |
| pH 7; S5204; | 0.38 | 5.04 | 2.39 | 88.02 | 2.39 | 0.44 |
| T672; | | | | | | |
| D2089-7 | | | | | | |
| pH 7; S5204; | 0.38 | 5.00 | 2.37 | 87.93 | 2.42 | 0.43 |
| T672; | | | | | | |
| D2089-18 | | | | | | |

TABLE 25
Fatty acid profile in some representative complemented
(D2096) and parent S5204 lines transformed with pSZ3384
DNA containing PmCPS1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 7; S5204; | 0.33 | 4.18 | 1.10 | 92.91 | 0.00 | 0.00 |
| T673; | | | | | | |
| D2096-6 | | | | | | |
| pH 7; S5204; | 0.36 | 4.14 | 1.33 | 92.42 | 0.34 | 0.12 |
| T673; | | | | | | |
| D2096-12 | | | | | | |
| pH 7; S5204; | 0.32 | 4.35 | 1.64 | 92.12 | 0.35 | 0.14 |
| T673; | | | | | | |
| D2096-14 | | | | | | |
| pH 7; S5204; | 0.50 | 6.44 | 0.95 | 89.81 | 0.46 | 0.32 |
| T673; | | | | | | |
| D2096-8 | | | | | | |
| pH 7; S5204; | 0.29 | 3.93 | 1.79 | 91.19 | 1.34 | 0.37 |
| T673; | | | | | | |
| D2096-1 | | | | | | |

TABLE 26
Fatty acid profile in some representative complemented
(D2090) and parent S5204 lines transformed with pSZ3378
DNA containing PmCPS2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 7; S5204; | 0.33 | 4.73 | 1.84 | 91.24 | 0.00 | 0.00 |
| T672; | | | | | | |
| D2090-5 | | | | | | |
| pH 7; S5204; | 0.42 | 4.99 | 2.01 | 91.06 | 0.00 | 0.00 |
| T672; | | | | | | |
| D2090-29 | | | | | | |
| pH 7; S5204; | 0.43 | 4.31 | 1.87 | 90.44 | 0.78 | 0.16 |
| T672; | | | | | | |
| D2090-22 | | | | | | |
| pH 7; S5204; | 0.32 | 3.77 | 2.43 | 89.72 | 1.68 | 0.35 |
| T672; | | | | | | |
| D2090-1 | | | | | | |
| pH 7; S5204; | 0.49 | 5.01 | 1.97 | 88.48 | 1.84 | 0.38 |
| T672; | | | | | | |
| D2090-32 | | | | | | |

TABLE 27
Fatty acid profile in some representative complemented
(D2097) and parent S5204 lines transformed with pSZ3385
DNA containing PmCPS2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 5; S5204; | 0.50 | 5.73 | 1.97 | 87.12 | 2.61 | 0.76 |
| T680; | | | | | | |
| D2097-1 | | | | | | |
| pH 5; S5204; | 0.75 | 8.20 | 2.46 | 85.73 | 0.89 | 0.53 |
| T680; | | | | | | |
| D2097-2 | | | | | | |

TABLE 28
Fatty acid profile in some representative complemented
(D2091) and parent S5204 lines transformed with pSZ3379
DNA containing PmDPS1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 7; S5204; | 1.42 | 4.39 | 2.32 | 89.87 | 0.00 | 0.00 |
| T672; | | | | | | |
| D2091-4 | | | | | | |
| pH 7; S5204; | 0.27 | 4.79 | 2.24 | 90.94 | 0.00 | 0.00 |
| T672; | | | | | | |
| D2091-14 | | | | | | |
| pH 7; S5204; | 0.30 | 5.26 | 2.20 | 90.73 | 0.00 | 0.00 |
| T672; | | | | | | |
| D2091-15 | | | | | | |
| pH 7; S5204; | 0.31 | 4.51 | 1.77 | 91.65 | 0.00 | 0.00 |
| T672; | | | | | | |
| D2091-19 | | | | | | |
| pH 7; S5204; | 0.31 | 5.36 | 2.24 | 90.67 | 0.00 | 0.00 |
| T672; | | | | | | |
| D2091-46 | | | | | | |

TABLE 29
Fatty acid profile in some representative complemented
(D2098) and parent S5204 lines transformed with pSZ3386
DNA containing PmDPS1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 7; S5204; | 0.34 | 4.89 | 1.56 | 92.08 | 0.00 | 0.00 |
| T680; | | | | | | |
| D2098-39 | | | | | | |
| pH 7; S5204; | 0.30 | 4.31 | 1.61 | 92.34 | 0.30 | 0.00 |
| T680; | | | | | | |
| D2098-7 | | | | | | |
| pH 7; S5204; | 0.33 | 3.89 | 1.58 | 92.65 | 0.36 | 0.00 |
| T680; | | | | | | |
| D2098-3 | | | | | | |
| pH 7; S5204; | 0.32 | 4.18 | 1.64 | 92.34 | 0.36 | 0.11 |
| T680; | | | | | | |
| D2098-25 | | | | | | |
| pH 7; S5204; | 0.32 | 4.36 | 1.50 | 92.10 | 0.37 | 0.12 |
| T680; | | | | | | |
| D2098-13 | | | | | | |

TABLE 30
Fatty acid profile in some representative complemented
(D2092) and parent S5204 lines transformed with pSZ3380
DNA containing PmDPS2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 7; S5204; | 0.29 | 5.13 | 1.59 | 92.16 | 0.00 | 0.00 |
| T672; | | | | | | |
| D2092-35 | | | | | | |
| pH 7; S5204; | 0.37 | 4.66 | 1.75 | 91.71 | 0.19 | 0.05 |
| T672; | | | | | | |
| D2092-29 | | | | | | |
| pH 7; S5204; | 0.24 | 3.47 | 1.84 | 93.19 | 0.43 | 0.11 |
| T672; | | | | | | |
| D2092-15 | | | | | | |
| pH 7; S5204; | 0.25 | 3.50 | 1.82 | 93.16 | 0.44 | 0.09 |
| T672; | | | | | | |
| D2092-21 | | | | | | |
| pH 7; S5204; | 0.28 | 3.18 | 1.50 | 93.59 | 0.52 | 0.12 |
| T672; | | | | | | |
| D2092-16 | | | | | | |

TABLE 31
Fatty acid profile in some representative complemented
(D2099) and parent S5204 lines transformed with pSZ3387
DNA containing PmDPS2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 7; S5204; | 0.31 | 4.02 | 1.46 | 93.07 | 0.00 | 0.00 |
| T680; | | | | | | |
| D2099-20 | | | | | | |
| pH 7; S5204; | 0.28 | 4.67 | 1.50 | 92.38 | 0.00 | 0.00 |
| T680; | | | | | | |
| D2099-24 | | | | | | |
| pH 7; S5204; | 0.40 | 4.07 | 1.22 | 93.26 | 0.00 | 0.00 |
| T680; | | | | | | |
| D2099-27 | | | | | | |
| pH 7; S5204; | 0.32 | 4.59 | 1.57 | 92.40 | 0.00 | 0.00 |
| T680; | | | | | | |
| D2099-30 | | | | | | |
| pH 7; S5204; | 0.30 | 4.56 | 1.54 | 92.49 | 0.00 | 0.00 |
| T680; | | | | | | |
| D2099-35 | | | | | | |

TABLE 32
Fatty acid profile in some representative complemented
(D2259) and parent S5204 lines transformed with pSZ3480
DNA containing PmIPP1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 5; S5204; | 0.36 | 5.27 | 2.19 | 89.32 | 1.51 | 0.51 |
| T711; | | | | | | |
| D2259-43 | | | | | | |
| pH 5; S5204; | 0.35 | 4.88 | 2.17 | 86.34 | 4.41 | 0.70 |
| T711; | | | | | | |
| D2259-22 | | | | | | |
| pH 5; S5204; | 0.35 | 4.82 | 2.18 | 86.32 | 4.45 | 0.69 |
| T711; | | | | | | |
| D2259-28 | | | | | | |
| pH 5; S5204; | 0.33 | 4.90 | 2.08 | 86.33 | 4.49 | 0.74 |
| T711; | | | | | | |
| D2259-21 | | | | | | |
| pH 5; S5204; | 0.50 | 5.97 | 2.14 | 84.67 | 4.49 | 0.74 |
| T711; | | | | | | |
| D2259-36 | | | | | | |

TABLE 33
Fatty acid profile in some representative complemented
(D2260) and parent S5204 lines transformed with pSZ3481
DNA containing PmIPP1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.10 | 0.00 |
| pH 5; S5204 | 0.39 | 5.67 | 1.36 | 91.13 | 0.00 | 0.00 |
| pH 5; S5204; | 0.36 | 4.96 | 2.10 | 89.46 | 1.55 | 0.49 |
| T711; | | | | | | |
| D2260-32 | | | | | | |
| pH 5; S5204; | 0.33 | 4.83 | 1.99 | 89.40 | 1.63 | 0.58 |
| T711; | | | | | | |
| D2260-10 | | | | | | |
| pH 5; S5204; | 0.34 | 4.83 | 2.16 | 89.39 | 1.64 | 0.49 |
| T711; | | | | | | |
| D2260-2 | | | | | | |
| pH 5; S5204; | 0.37 | 4.81 | 2.11 | 89.51 | 1.69 | 0.26 |
| T711; | | | | | | |
| D2260-30 | | | | | | |
| pH 5; S5204; | 0.33 | 4.91 | 2.17 | 89.73 | 1.72 | 0.16 |
| T711; | | | | | | |
| D2260-41 | | | | | | |

TABLE 34
Fatty acid profile in some representative complemented
(D2434) and parent S5204 lines transformed with pSZ3509
DNA containing PmAHC1p driving PmFAD2-1.

| Sample ID | C14.0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; | 0.33 | 4.45 | 1.55 | 81.55 | 8.51 | 1.38 |
| T768; | | | | | | |
| D2434-32 | | | | | | |
| pH 5; S5204; | 0.62 | 7.27 | 1.58 | 78.65 | 9.44 | 1.49 |
| T768; | | | | | | |
| D2434-27 | | | | | | |
| pH 5; S5204; | 0.38 | 5.81 | 1.79 | 79.63 | 10.01 | 1.18 |
| T768; | | | | | | |
| D2434-4 | | | | | | |
| pH 5; S5204; | 0.5 | 5.93 | 1.5 | 78.7 | 10.25 | 1.56 |
| T768; | | | | | | |
| D2434-23 | | | | | | |
| pH 5; S5204; | 0.51 | 6.08 | 1.6 | 78.79 | 10.25 | 1.36 |
| T768; | | | | | | |
| D2434-43 | | | | | | |

TABLE 35
Fatty acid profile in some representative complemented (D2266) and
parent S5204 lines transformed with pSZ3516 DNA containing
PmAHC1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T718; D2266-46 | 0.32 | 5.41 | 1.94 | 91.26 | 0.11 | 0.00 |
| pH 5; S5204; T718; D2266-36 | 0.36 | 5.33 | 1.90 | 91.17 | 0.17 | 0.00 |
| pH 5; S5204; T718; D2266-35 | 0.37 | 4.96 | 2.13 | 90.82 | 0.41 | 0.00 |
| pH 5; S5204; T718; D2266-41 | 0.38 | 5.33 | 2.10 | 90.31 | 0.44 | 0.31 |
| pH 5; S5204; T718; D2266-5 | 0.36 | 5.15 | 2.23 | 90.55 | 0.48 | 0.31 |

TABLE 36
Fatty acid profile in some representative complemented (D2435) and
parent S5204 lines transformed with pSZ3510 DNA containing
PmAHC2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T768; D2435-37 | 0.35 | 6.09 | 1.90 | 78.52 | 11.01 | 1.18 |
| pH 5; S5204; T768; D2435-3 | 0.43 | 5.90 | 1.97 | 78.74 | 10.97 | 1.20 |
| pH 5; S5204; T768; D2435-20 | 0.40 | 6.01 | 1.89 | 79.00 | 10.97 | 1.14 |
| pH 5; S5204; T768; D2435-13 | 0.39 | 6.11 | 1.89 | 78.26 | 10.84 | 1.24 |
| pH 5; S5204; T768; D2435-34 | 0.46 | 6.02 | 1.97 | 79.48 | 10.46 | 1.19 |

TABLE 37
Fatty acid profile in some representative complemented (D2263) and
parent S5204 lines transformed with pSZ3513 DNA containing
PmPPI1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T718; D2263-13 | 0.75 | 9.44 | 1.98 | 87.09 | 0.00 | 0.00 |
| pH 5; S5204; T718; D2263-14 | 0.58 | 7.72 | 1.64 | 89.26 | 0.00 | 0.00 |
| pH 5; S5204; T718; D2263-19 | 0.62 | 7.92 | 1.56 | 89.25 | 0.00 | 0.00 |
| pH 5; S5204; T718; D2263-26 | 0.42 | 7.39 | 1.70 | 89.28 | 0.00 | 0.00 |
| pH 5; S5204; T718; D2263-29 | 0.58 | 7.32 | 1.30 | 90.07 | 0.00 | 0.00 |

TABLE 38
Fatty acid profile in some representative complemented (D2440) and
parent S5204 lines transformed with pSZ3689 DNA containing
PmPPI1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T770; D2440-23 | 0.31 | 6.24 | 1.41 | 90.42 | 0.17 | 0.05 |
| pH 5; S5204; T770; D2440-32 | 0.23 | 4.69 | 1.41 | 91.72 | 0.17 | 0.00 |
| pH 5; S5204; T770; D2440-38 | 0.30 | 6.31 | 1.49 | 90.21 | 0.17 | 0.00 |
| pH 5; S5204; T770; D2440-7 | 0.30 | 6.33 | 1.38 | 90.29 | 0.18 | 0.05 |
| pH 5; S5204; T770; D2440-36 | 0.29 | 6.38 | 1.36 | 90.39 | 0.18 | 0.05 |
| pH 5; S5204; T770; D2440-8 | 0.34 | 5.63 | 1.15 | 91.15 | 0.19 | 0.05 |

TABLE 39
Fatty acid profile in some representative complemented (D2264) and
parent S5204 lines transformed with pSZ3514 DNA containing
PmPPI2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 7; S6207; T718; D2264-1 | 0.49 | 6.15 | 1.61 | 90.82 | 0.00 | 0.00 |
| pH 7; S6207; T718; D2264-6 | 0.38 | 5.36 | 1.51 | 91.58 | 0.00 | 0.00 |
| pH 7; S6207; T718; D2264-29 | 0.45 | 6.09 | 1.46 | 91.10 | 0.00 | 0.00 |
| pH 7; S6207; T718; D2264-4 | 0.40 | 5.42 | 2.28 | 89.86 | 0.90 | 0.00 |
| pH 7; S6207; T718; D2264-7 | 0.40 | 5.37 | 2.02 | 90.18 | 1.04 | 0.00 |

TABLE 40
Fatty acid profile in some representative complemented (D2268) and
parent S5204 lines transformed with pSZ3518 DNA containing
PmPPI2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T720; D2268-1 | 0.39 | 6.43 | 1.78 | 90.49 | 0.00 | 0.00 |
| pH 5; S5204; T720; D2268-2 | 0.38 | 6.49 | 1.74 | 90.38 | 0.00 | 0.00 |
| pH 5; S5204; T720; D2268-3 | 0.38 | 6.56 | 1.74 | 90.27 | 0.00 | 0.00 |
| pH 5; S5204; T720; D2268-4 | 0.45 | 5.73 | 1.52 | 91.75 | 0.00 | 0.00 |
| pH 5; S5204; T720; D2268-5 | 0.38 | 6.58 | 1.81 | 90.79 | 0.00 | 0.00 |

TABLE 41
Fatty acid profile in some representative complemented (D2265) and
parent S5204 lines transformed with pSZ3515 DNA containing
PmGMPS1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T718; D2265-16 | 0.46 | 7.02 | 1.71 | 90.06 | 0.00 | 0.00 |
| pH 5; S5204; T718; D2265-43 | 0.00 | 7.90 | 1.90 | 89.27 | 0.00 | 0.00 |
| pH 5; S5204; T718; D2265-14 | 0.46 | 5.53 | 1.68 | 91.28 | 0.35 | 0.00 |
| pH 5; S5204; T718; D2265-4 | 0.39 | 6.17 | 1.75 | 90.44 | 0.42 | 0.00 |
| pH 5; S5204; T718; D2265-9 | 0.49 | 5.87 | 1.77 | 90.51 | 0.45 | 0.00 |

TABLE 42
Fatty acid profile in some representative complemented (D2269) and
parent S5204 lines transformed with pSZ3519 DNA containing
PmGMPS1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T720; D2269-1 | 0.38 | 6.73 | 1.68 | 90.24 | 0.00 | 0.00 |
| pH 5; S5204; T720; D2269-3 | 0.36 | 6.76 | 1.71 | 90.17 | 0.00 | 0.00 |
| pH 5; S5204; T720; D2269-4 | 0.42 | 6.57 | 1.71 | 90.32 | 0.00 | 0.00 |
| pH 5; S5204; T720; D2269-5 | 0.59 | 8.81 | 1.93 | 87.97 | 0.00 | 0.00 |
| pH 5; S5204; T720; D2269-6 | 0.50 | 7.29 | 1.73 | 89.29 | 0.00 | 0.00 |

TABLE 43
Fatty acid profile in some representative complemented (D2270) and
parent S5204 lines transformed with pSZ3520 DNA containing
PmGMPS2p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T720; D2270-1 | 0.37 | 6.80 | 1.74 | 90.18 | 0.00 | 0.00 |
| pH 5; S5204; T720; D2270-2 | 0.46 | 6.76 | 1.83 | 89.90 | 0.00 | 0.00 |
| pH 5; S5204; T720; D2270-3 | 0.41 | 6.69 | 1.70 | 90.22 | 0.00 | 0.00 |
| pH 5; S5204; T720; D2270-4 | 0.43 | 7.44 | 1.72 | 89.31 | 0.00 | 0.00 |
| pH 5; S5204; T720; D2270-5 | 0.44 | 6.98 | 1.78 | 89.79 | 0.00 | 0.00 |

TABLE 44
Fatty acid profile in some representative complemented (D2436) and
parent S5204 lines transformed with pSZ3684 DNA containing PmCS1p
driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T768; D2436-48 | 7.59 | 1.57 | 88.88 | 0.18 | 0.00 | 0.00 |
| pH 5; S5204; T768; D2436-1 | 6.37 | 1.50 | 85.00 | 3.97 | 1.04 | 0.00 |
| pH 5; S5204; T768; D2436-16 | 9.40 | 1.86 | 81.13 | 4.11 | 1.21 | 0.00 |
| pH 5; S5204; T768; D2436-8 | 6.07 | 1.77 | 84.78 | 4.26 | 0.94 | 0.00 |
| pH 5; S5204; T768; D2436-32 | 5.97 | 1.62 | 85.28 | 4.50 | 0.98 | 0.00 |

TABLE 45
Fatty acid profile in some representative complemented (D2438) and
parent S5204 lines transformed with pSZ3686 DNA containing
PmCS1p driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T770; D2438-7 | 0.50 | 5.96 | 1.69 | 89.87 | 1.30 | 0.00 |
| pH 5; S5204; T770; D2438-11 | 0.41 | 6.05 | 1.86 | 87.88 | 2.46 | 0.00 |
| pH 5; S5204; T770; D2438-9 | 0.41 | 5.75 | 1.93 | 88.35 | 2.50 | 0.00 |
| pH 5; S5204; T770; D2438-15 | 0.45 | 6.18 | 1.85 | 87.86 | 2.59 | 0.00 |
| pH 5; S5204; T770; D2438-37 | 0.40 | 5.92 | 1.97 | 87.80 | 2.59 | 0.00 |

TABLE 46
Fatty acid profile in some representative complemented (D2437) and
parent S5204 lines transformed with pSZ3685 DNA containing
PmCSCp driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T768; D2437-15 | 0.00 | 4.83 | 1.98 | 90.43 | 1.17 | 0.53 |
| pH 5; S5204; T768; D2437-35 | 0.45 | 6.03 | 1.81 | 88.69 | 1.88 | 0.31 |
| pH 5; S5204; T768; D2437-17 | 0.39 | 4.96 | 2.00 | 88.58 | 3.24 | 0.00 |
| pH 5; S5204; T768; D2437-26 | 0.90 | 9.55 | 2.07 | 82.29 | 3.37 | 1.24 |
| pH 5; S5204; T768; D2437-8 | 0.53 | 10.76 | 1.55 | 79.62 | 4.46 | 1.12 |

TABLE 47
Fatty acid profile in some representative complemented (D2439) and
parent S5204 lines transformed with pSZ3688 DNA containing PmGGHp
driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T770; D2439-11 | 0.31 | 6.79 | 1.47 | 89.97 | 0.00 | 0.00 |
| pH 5; S5204; T770; D2439-22 | 0.27 | 4.19 | 0.94 | 92.91 | 0.08 | 0.00 |
| pH 5; S5204; T770; D2439-12 | 0.39 | 6.02 | 1.26 | 90.91 | 0.16 | 0.00 |
| pH 5; S5204; T770; D2439-34 | 0.64 | 6.50 | 1.10 | 89.53 | 0.20 | 0.00 |
| pH 5; S5204; T770; D2439-32 | 0.33 | 5.25 | 1.45 | 89.98 | 1.08 | 0.51 |

TABLE 48
Fatty acid profile in some representative complemented (D2261) and
parent S5204 lines transformed with pSZ3511 DNA containing PmAHI2p
driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T711; D2261-35 | 0.45 | 5.06 | 2.02 | 89.35 | 1.73 | 0.63 |
| pH 5; S5204; T711; D2261-8 | 0.46 | 5.12 | 2.19 | 88.92 | 2.16 | 0.19 |
| pH 5; S5204; T711; D2261-43 | 0.37 | 5.12 | 2.15 | 88.62 | 2.30 | 0.45 |
| pH 5; S5204; T711; D2261-2 | 0.42 | 5.27 | 2.14 | 88.23 | 2.39 | 0.30 |
| pH 5; S5204; T711; D2261-24 | 0.41 | 5.14 | 2.23 | 88.44 | 2.39 | 0.45 |

TABLE 49
Fatty acid profile in some representative complemented (D2267) and
parent S5204 lines transformed with pSZ3517 DNA containing PmAHI1p
driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; T720; D2267-3 | 0.34 | 4.87 | 2.11 | 90.00 | 1.20 | 0.39 |
| pH 5; S5204; T720; D2267-20 | 0.37 | 5.00 | 2.14 | 89.50 | 1.46 | 0.49 |
| pH 5; S5204; T720; D2267-36 | 0.34 | 4.90 | 2.08 | 89.75 | 1.67 | 0.36 |
| pH 5; S5204; T720; D2267-15 | 0.37 | 4.95 | 2.14 | 89.77 | 1.69 | 0.00 |
| pH 5; S5204; T720; D2267-2 | 0.35 | 4.85 | 2.12 | 89.71 | 1.72 | 0.32 |

TABLE 50
Fatty acid profile in some representative complemented (D2262) and
parent S5204 lines transformed with pSZ3512 DNA containing PmCEP1p
driving PmFAD2-1.

| Sample ID | C14:0 | C16:0 | C18:0 | C18:1 | C18:2 | C18:3 α |
|---|---|---|---|---|---|---|
| pH 7; S3150 | 1.71 | 29.58 | 3.13 | 56.53 | 6.43 | 0.68 |
| pH 5; S3150 | 1.56 | 27.70 | 2.98 | 59.49 | 5.95 | 0.53 |
| pH 7; S5204 | 0.30 | 5.59 | 1.63 | 90.88 | 0.1 | 0 |
| pH 5; S5204 | 0.39 | 5.67 | 1.36 | 91.13 | 0 | 0 |
| pH 5; S5204; | 0.48 | 5.50 | 2.08 | 90.58 | 0.35 | 0.00 |
| T711; | | | | | | |
| D2262-3 | | | | | | |
| pH 5; S5204; | 0.39 | 5.20 | 2.17 | 89.90 | 1.08 | 0.37 |
| T711; | | | | | | |
| D2262-33 | | | | | | |
| pH 5; S5204; | 0.34 | 5.08 | 1.93 | 89.69 | 1.34 | 0.37 |
| T711; | | | | | | |
| D2262-24 | | | | | | |
| pH 5; S5204; | 0.40 | 4.89 | 2.19 | 89.88 | 1.45 | 0.27 |
| T711; | | | | | | |
| D2262-32 | | | | | | |
| pH 5; S5204; | 0.39 | 4.95 | 2.75 | 89.30 | 1.47 | 0.27 |
| T711; | | | | | | |
| D2262-34 | | | | | | |

Combined baseline expression of endogenous PmFAD2-1 and PmFAD2-2 in wild type Prototheca strains (like S3150, S1920 or S1331) manifests as 5-7% C18:2. S5204 overexpresses PmKASII which results in the elongation of C16:0 to C18:0. This increased pool of C18:0 is eventually desaturated by PmSAD2 resulting in elevated C18:1 levels. Additionally disruption of the both copies of PmFAD2 (viz. PmFAD2-1 and PmFAD2-2) in S5204 prevents further desaturation of C18:1 into C18:2 and results in a unique high oleic oil (C18:1) with 0% linoleic acid (C18:2). However as mentioned above any strain with 0% C18:2 grows very poorly and requires exogenous addition of linoleic acid to sustain growth/productivity. Complementation of a strain like S5204 with inducible PmAMT03p driven PmFAD2-1 can rescue the growth phenotype while preserving the terminal high C18:1 with 0% C18:2 levels. However data suggests that PmAMT03 shuts off in the early stages of fermentation thus severely compromising the ability of any complemented strain to achieve its full growth and productivity potential. The goal of this work was to identify promoter elements that would allow the complemented strains to grow efficiently in early stages of fermentation (T0-T30 hrs; irrespective of excess batched N in the fermenters) and then effectively shut off once the cells enter active lipid production (when N in the media gets depleted) so that the complemented strains would still finish with very high C18:1 and 0% C18:2 levels. As a comparator we also complemented S5204 with PmFAD2-1 being driven by either PmFAd2-1p or PmFAD2-2p promoter elements.

Complementation of S5204 with PmFAD2-1 driven by either PmFAD2-1p or PmFAD2-2p promoter elements results in complete restoration of the C18:2 levels using vectors either designed to amplify PmFAD2-1 copy number (e.g. pSZ3375 or pSZ3376) or the ones where PmFAD2-1 copy number is restricted to one (pSZ3382 or pSZ3383). Copy number of the PmFAD2-1 in these strains seems to have very marginal effect on the terminal C18:2 levels.

On the other hand expression of PmFAD2-1 driven by any of new promoter elements results in marked decrease in terminal C18:2 levels. The representative profiles from various strains expressing new promoters driving FAD2-1 are shown in Tables 20-50. This reduction in C18:2 levels is even more pronounced in strains where the copy number of PmFAD2-1 is limited to one. Promoter elements like PmDPS1 (D2091 & D2098), PmDPS2 (D2092 & D2099), PmPPI1 (D2263 & D2440), PmPPI2 (D2264 & D2268), PmGMPS1 (D2265 & D2269), PmGMPS2 (D2270) resulted in strains with 0% or less than 0.5% terminal C18:2 levels in both single or multiple copy PmFAD2-1 versions. The rest of the promoters resulted in terminal C18:2 levels that ranged between 1-5%. One unexpected result was the data from PmAHC1p and PmAHC2p driving PmFAD2-1 in D2434 and D2435. Both these promoters resulted in very high levels of C18:2 (9-20%) in multiple copy FAD2-1 versions. The levels of terminal C18:2 in single copy version in D2266 was more in line with the transcriptomic data suggesting that PmAHC promoter activity and the corresponding PmAHC transcription is severely downregulated when cells are actively producing lipid in depleted nitrogen environment. A quick look at the transcriptome revealed that the initial transcription of PmAHC is very high (4000-5500 TPM) which then suddenly drops down to ˜250 TPM. Thus it is conceivable that in strains with multiple copies on PmFAD2-1 (D2434 and D2435), the massive amount of PmFAD2-1 protein produced earlier in the fermentation lingers and results in high C18:2 levels. In single copy PmFAD2-1 strains this is not the case and thus we do not see elevated C18:2 levels in D2266.

In complemented strains with 0% terminal C18:2 levels, the key question was whether they were complemented in the first place. In order to ascertain that, representative strains along with parent S5204 and previously AMT03p driven PmFAD2-1 complemented S2532 (viz S4695) strains were grown in seed medium in 96 well blocks. The cultures were seeded at 0.1 OD units per ml and the OD750 was checked at different time points. Compared to S5204, which grew very poorly, only S4695 and newly complemented strains grew to any meaningful OD's at 20 and 44 hrs (Table 51) demonstrating that the promoters identified above are active early on and switch off once cells enter the active lipid production phase.

TABLE 51
Growth characteristics of Δfad2 Δfata1 strain S5204, S4695 and
representative complemented S5204 lines in seed medium sorted by OD750 at 44 hrs. Note
that in 1 ml 96 well blocks after initial rapid division and growth, cells stop growing
efficiently because of lack of nutrients, aeration etc.

| | | | | | | | OD750 | OD750 | OD750 |
|---|---|---|---|---|---|---|---|---|---|
| Sample ID | C14:0 | C16:0 | C18:0 | C18:1 | C18:2 | C18:3 α | @20 hrs | @44 hrs | @68 hrs |
| S5204 | | | | | | | 0.162 | 7.914 | 10.93 |
| S5204 | | | | | | | 0.224 | 6.854 | 9.256 |
| S4695 | | | | | | | 1.456 | 29.032 | 32.766 |
| pH 7; S5204; T672; D2091-46 | 0.31 | 5.36 | 2.24 | 90.67 | 0.00 | 0.00 | 1.38 | 33.644 | 33.226 |
| pH 5; S5204; T720; D2268-1 | 0.39 | 6.43 | 1.78 | 90.49 | 0.00 | 0.00 | 0.75 | 32.782 | 31.624 |
| S5204; T720; D2270-47 | 0.39 | 6.69 | 1.81 | 90.05 | 0.00 | 0.00 | 1.204 | 32.752 | 31.602 |
| pH 5; S5204; T720; D2270-39 | 0.39 | 6.87 | 1.81 | 89.94 | 0.00 | 0.00 | 1.012 | 32.552 | 33.138 |
| pH 7; S5204; T680; D2099-35 | 0.30 | 4.56 | 1.54 | 92.49 | 0.00 | 0.00 | 0.48 | 32.088 | 31.92 |
| pH 5; S5204; T720; D2270-44 | 0.51 | 6.85 | 1.74 | 90.06 | 0.00 | 0.00 | 1.468 | 31.802 | 30.61 |
| pH 5; S5204; T720; D2270-41 | 0.00 | 7.85 | 1.65 | 89.18 | 0.00 | 0.00 | 1.576 | 31.35 | 30.69 |
| pH 5; S5204; T720; D2270-17 | 0.46 | 6.78 | 1.71 | 90.24 | 0.00 | 0.00 | 1.79 | 30.732 | 24.768 |
| pH 7; S5204; T680; D2099-30 | 0.32 | 4.59 | 1.57 | 92.40 | 0.00 | 0.00 | 0.59 | 30.166 | 34.64 |
| pH 5; S5204; T720; D2268-40 | 0.42 | 6.66 | 1.86 | 90.02 | 0.00 | 0.00 | 0.764 | 29.62 | 29 |
| pH 5; S5204; T720; D2270-23 | 0.39 | 6.52 | 1.72 | 90.35 | 0.00 | 0.00 | 1.334 | 29.604 | 27.518 |
| pH 5; S5204; T720; D2270-42 | 0.61 | 6.59 | 1.53 | 90.28 | 0.00 | 0.00 | 2.042 | 28.986 | 32.184 |
| pH 7; S5204; T672; D2090-5 | 0.33 | 4.73 | 1.84 | 91.24 | 0.00 | 0.00 | 1.326 | 28.976 | 35.508 |
| pH 7; S5204; T672; D2091-15 | 0.30 | 5.26 | 2.20 | 90.73 | 0.00 | 0.00 | 0.826 | 28.824 | 32.848 |
| pH 7; S5204; T680; D2099-20 | 0.31 | 4.02 | 1.46 | 93.07 | 0.00 | 0.00 | 1.31 | 28.732 | 26.61 |
| pH 5; S5204; T720; D2269-19 | 0.42 | 6.51 | 1.61 | 90.43 | 0.00 | 0.00 | 1.278 | 28.65 | 31.362 |
| pH 5; S5204; T720; D2269-29 | 0.43 | 7.36 | 1.72 | 89.35 | 0.00 | 0.00 | 1.342 | 28.376 | 28.66 |
| pH 5; S5204; T720; D2270-19 | 0.39 | 6.81 | 1.75 | 90.05 | 0.00 | 0.00 | 2.142 | 28.376 | 25.934 |
| pH 5; S5204; T720; D2270-43 | 0.80 | 7.64 | 1.66 | 88.93 | 0.00 | 0.00 | 1.896 | 28.174 | 32.376 |
| pH 5; S5204; T720; D2270-46 | 0.45 | 6.75 | 1.72 | 90.02 | 0.00 | 0.00 | 1.644 | 28.122 | 30.464 |
| pH 5; S5204; T720; D2268-3 | 0.38 | 6.56 | 1.74 | 90.27 | 0.00 | 0.00 | 0.926 | 28.114 | 31.552 |
| pH 5; S5204; T720; D2268-12 | 0.00 | 5.68 | 1.84 | 91.53 | 0.00 | 0.00 | 1.414 | 28.106 | 30.644 |
| pH 5; S5204; T720; D2269-37 | 0.54 | 7.12 | 1.75 | 89.80 | 0.00 | 0.00 | 1.268 | 28.078 | 30.014 |
| pH 5; S5204; T720; D2270-31 | 0.46 | 6.94 | 1.74 | 89.71 | 0.00 | 0.00 | 1.224 | 28.064 | 29.344 |
| pH 5; S5204; T720; D2270-48 | 0.00 | 7.21 | 1.87 | 90.16 | 0.00 | 0.00 | 1.352 | 28 | 28.21 |
| pH 5; S5204; T720; D2269-8 | 0.33 | 6.67 | 1.64 | 90.34 | 0.00 | 0.00 | 0.96 | 27.912 | 27.564 |
| pH 5; S5204; T720; D2268-32 | 0.44 | 6.59 | 1.85 | 90.11 | 0.00 | 0.00 | 0.78 | 27.834 | 31.952 |
| pH 5; S5204; T720; D2269-47 | 0.42 | 6.83 | 1.82 | 89.85 | 0.00 | 0.00 | 1.17 | 27.76 | 29.648 |
| pH 7; S5204; T672; D2091-19 | 0.31 | 4.51 | 1.77 | 91.65 | 0.00 | 0.00 | 1.568 | 27.682 | 25.828 |
| pH 5; S5204; T720; D2270-38 | 0.39 | 6.65 | 1.83 | 90.11 | 0.00 | 0.00 | 1.74 | 27.606 | 31.104 |
| pH 5; S5204; T720; D2268-2 | 0.38 | 6.49 | 1.74 | 90.38 | 0.00 | 0.00 | 0.95 | 27.564 | 32.254 |
| pH 5; S5204; T720; D2269-35 | 0.38 | 7.04 | 1.68 | 89.82 | 0.00 | 0.00 | 1.19 | 27.482 | 29.186 |
| pH 5; S5204; T720; D2269-20 | 0.36 | 7.01 | 1.73 | 89.86 | 0.00 | 0.00 | 0.966 | 27.47 | 28.284 |
| pH 5; S5204; T720; D2269-13 | 0.39 | 6.76 | 1.89 | 89.98 | 0.00 | 0.00 | 0.936 | 27.39 | 33.464 |
| pH 7; S5204; T680; D2099-24 | 0.28 | 4.67 | 1.50 | 92.38 | 0.00 | 0.00 | 0.8 | 27.28 | 27.35 |
| pH 5; S5204; T720; D2268-11 | 0.38 | 6.56 | 1.85 | 90.56 | 0.00 | 0.00 | 1.136 | 27.254 | 32.508 |
| pH 5; S5204; T720; D2270-3 | 0.41 | 6.69 | 1.70 | 90.22 | 0.00 | 0.00 | 0.872 | 27.214 | 30.23 |
| pH 5; S5204; T720; D2269-33 | 0.39 | 6.36 | 1.67 | 90.59 | 0.00 | 0.00 | 0.956 | 27.194 | 30.568 |
| pH 5; S5204; T720; D2268-10 | 0.45 | 6.93 | 1.70 | 90.16 | 0.00 | 0.00 | 0.612 | 27.126 | 31.616 |
| pH 5; S5204; T720; D2269-43 | 0.36 | 6.55 | 1.84 | 90.25 | 0.00 | 0.00 | 0.998 | 27.086 | 29.618 |
| pH 5; S5204; T720; D2270-1 | 0.37 | 6.80 | 1.74 | 90.18 | 0.00 | 0.00 | 2.428 | 27.004 | 31.044 |
| pH 5; S5204; T720; D2268-4 | 0.45 | 5.73 | 1.52 | 91.75 | 0.00 | 0.00 | 0.736 | 26.948 | 28.796 |
| pH 5; S5204; T720; D2270-9 | 0.38 | 6.88 | 1.74 | 90.22 | 0.00 | 0.00 | 2.68 | 26.944 | 29.92 |
| pH 5; S5204; T720; D2269-26 | 0.41 | 6.85 | 1.68 | 90.03 | 0.00 | 0.00 | 0.896 | 26.794 | 31.31 |
| pH 5; S5204; T720; D2270-24 | 0.39 | 6.51 | 1.78 | 90.33 | 0.00 | 0.00 | 1.51 | 26.682 | 27.486 |
| pH 5; S5204; T720; D2269-18 | 0.41 | 7.04 | 1.71 | 89.83 | 0.00 | 0.00 | 1.024 | 26.58 | 29.794 |
| pH 5; S5204; T720; D2269-32 | 0.38 | 6.81 | 1.72 | 90.06 | 0.00 | 0.00 | 1.214 | 26.48 | 29.478 |
| pH 5; S5204; T720; D2268-31 | 0.33 | 6.68 | 1.76 | 90.20 | 0.00 | 0.00 | 0.808 | 26.432 | 31.294 |
| pH 5; S5204; T720; D2269-7 | 0.29 | 5.33 | 1.69 | 91.59 | 0.00 | 0.00 | 1.1 | 26.41 | 28.754 |
| pH 5; S5204; T720; D2268-6 | 0.39 | 6.62 | 1.70 | 90.28 | 0.00 | 0.00 | 0.626 | 26.372 | 30.822 |
| pH 7; S5204; T680; D2099-27 | 0.40 | 4.07 | 1.22 | 93.26 | 0.00 | 0.00 | 0.936 | 26.116 | 29.75 |
| pH 5; S5204; T720; D2269-39 | 0.48 | 6.88 | 1.82 | 89.67 | 0.00 | 0.00 | 2.218 | 26.106 | 30.8 |
| pH 5; S5204; T720; D2269-12 | 0.35 | 6.39 | 1.80 | 90.47 | 0.00 | 0.00 | 1.18 | 26.032 | 28.19 |
| pH 5; S5204; T720; D2269-42 | 0.39 | 6.99 | 1.67 | 89.91 | 0.00 | 0.00 | 2.132 | 25.924 | 27.854 |
| pH 5; S5204; T720; D2268-8 | 0.56 | 6.77 | 1.49 | 90.20 | 0.00 | 0.00 | 0.96 | 25.702 | 29.788 |
| pH 5; S5204; T720; D2270-37 | 0.44 | 7.33 | 1.71 | 89.69 | 0.00 | 0.00 | 0.916 | 25.612 | 34.034 |
| pH 5; S5204; T720; D2270-40 | 0.00 | 9.30 | 1.62 | 88.12 | 0.00 | 0.00 | 2.072 | 25.552 | 29.474 |
| pH 5; S5204; T720; D2270-14 | 0.43 | 7.40 | 1.71 | 89.73 | 0.00 | 0.00 | 1.916 | 25.526 | 27.908 |
| pH 5; S5204; T720; D2269-21 | 0.40 | 6.69 | 1.69 | 89.99 | 0.00 | 0.00 | 0.826 | 25.396 | 29 |
| pH 5; S5204; T718; D2265-16 | 0.46 | 7.02 | 1.71 | 90.06 | 0.00 | 0.00 | 0.9 | 25.332 | 32.018 |
| pH 5; S5204; T720; D2270-15 | 0.40 | 6.90 | 1.68 | 90.32 | 0.00 | 0.00 | 1.594 | 25.32 | 26.794 |
| pH 5; S5204; T720; D2269-40 | 0.00 | 7.00 | 1.66 | 90.15 | 0.00 | 0.00 | 1.804 | 25.286 | 29.468 |
| pH 5; S5204; T720; D2268-5 | 0.38 | 6.58 | 1.81 | 90.79 | 0.00 | 0.00 | 0.678 | 25.156 | 33.066 |
| pH 5; S5204; T720; D2270-18 | 0.45 | 6.20 | 1.45 | 91.09 | 0.00 | 0.00 | 2.646 | 25.126 | 27.536 |
| pH 5; S5204; T720; D2269-25 | 0.44 | 7.02 | 1.69 | 89.91 | 0.00 | 0.00 | 0.868 | 25.018 | 32.104 |
| pH 5; S5204; T720; D2269-30 | 0.45 | 6.77 | 1.78 | 90.00 | 0.00 | 0.00 | 0.718 | 24.978 | 29.868 |
| pH 5; S5204; T720; D2270-25 | 0.31 | 6.82 | 1.68 | 90.09 | 0.00 | 0.00 | 2.32 | 24.814 | 36.024 |
| pH 5; S5204; T720; D2270-21 | 0.52 | 7.23 | 1.70 | 89.99 | 0.00 | 0.00 | 1.92 | 24.58 | 25.398 |
| pH 5; S5204; T720; D2269-38 | 0.00 | 7.45 | 1.50 | 90.19 | 0.00 | 0.00 | 1.494 | 24.578 | 30.178 |
| pH 5; S5204; T720; D2268-9 | 0.48 | 5.94 | 1.51 | 90.83 | 0.00 | 0.00 | 0.73 | 24.344 | 30.83 |
| pH 5; S5204; T720; D2268-37 | 0.44 | 6.35 | 1.84 | 90.31 | 0.00 | 0.00 | 0.548 | 24.306 | 32.848 |
| pH 5; S5204; T720; D2269-28 | 0.41 | 7.12 | 1.66 | 89.73 | 0.00 | 0.00 | 0.808 | 24.288 | 31.27 |
| pH 5; S5204; T720; D2270-5 | 0.44 | 6.98 | 1.78 | 89.79 | 0.00 | 0.00 | 2.328 | 24.14 | 30.186 |
| pH 5; S5204; T720; D2269-23 | 0.44 | 6.99 | 1.71 | 89.43 | 0.00 | 0.00 | 0.876 | 24.076 | 29.494 |
| pH 5; S5204; T720; D2269-9 | 0.38 | 6.84 | 1.71 | 90.32 | 0.00 | 0.00 | 0.806 | 24 | 26.844 |
| pH 5; S5204; T720; D2269-24 | 0.55 | 7.31 | 1.71 | 89.68 | 0.00 | 0.00 | 1.09 | 23.97 | 29.642 |
| pH 5; S5204; T720; D2270-35 | 0.36 | 6.58 | 1.72 | 90.38 | 0.00 | 0.00 | 1.554 | 23.71 | 28.868 |
| pH 5; S5204; T720; D2269-15 | 0.00 | 5.69 | 1.36 | 91.86 | 0.00 | 0.00 | 1.246 | 23.584 | 28.196 |
| pH 5; S5204; T720; D2270-28 | 0.39 | 7.15 | 1.82 | 89.92 | 0.00 | 0.00 | 1.648 | 23.486 | 30.858 |
| pH 7; S5204; T680; D2098-39 | 0.34 | 4.89 | 1.56 | 92.08 | 0.00 | 0.00 | 1.08 | 23.46 | 31.888 |
| pH 5; S5204; T720; D2269-27 | 0.33 | 6.87 | 1.68 | 89.98 | 0.00 | 0.00 | 1.3 | 23.262 | 33.112 |
| pH 5; S5204; T718; D2265-43 | 0.00 | 7.90 | 1.90 | 89.27 | 0.00 | 0.00 | 0.832 | 23.23 | 30.052 |
| pH 5; S5204; T720; D2270-30 | 0.41 | 7.00 | 1.68 | 89.83 | 0.00 | 0.00 | 2.144 | 23.1 | 30.97 |
| pH 5; S5204; T720; D2268-25 | 0.00 | 7.05 | 1.94 | 90.20 | 0.00 | 0.00 | 0.716 | 23.088 | 29.922 |
| pH 5; S5204; T720; D2270-29 | 0.34 | 6.81 | 1.74 | 90.11 | 0.00 | 0.00 | 2.542 | 22.98 | 31.402 |
| pH 5; S5204; T720; D2269-45 | 0.00 | 7.64 | 1.56 | 89.90 | 0.00 | 0.00 | 0.806 | 22.892 | 29.022 |
| pH 5; S5204; T720; D2270-27 | 0.72 | 9.32 | 1.99 | 87.35 | 0.00 | 0.00 | 2.352 | 22.81 | 29.996 |
| pH 5; S5204; T720; D2269-11 | 0.65 | 6.41 | 1.69 | 90.22 | 0.00 | 0.00 | 1.056 | 22.768 | 26.056 |
| pH 5; S5204; T720; D2270-36 | 0.00 | 5.45 | 1.59 | 91.60 | 0.00 | 0.00 | 1.886 | 22.738 | 24.69 |
| pH 5; S5204; T720; D2269-22 | 0.39 | 7.12 | 1.72 | 89.63 | 0.00 | 0.00 | 1.08 | 22.634 | 27.532 |
| pH 5; S5204; T718; D2263-30 | 0.54 | 7.58 | 1.57 | 89.47 | 0.00 | 0.00 | 0.71 | 22.564 | 29.996 |
| pH 7; S5204; T672; D2091-47 | 0.32 | 5.22 | 2.23 | 90.45 | 0.00 | 0.00 | 0.938 | 22.486 | 32.046 |
| pH 5; S5204; T720; D2269-1 | 0.38 | 6.73 | 1.68 | 90.24 | 0.00 | 0.00 | 1.154 | 22.48 | 29.994 |
| pH 7; S5204; T673; D2096-6 | 0.33 | 4.18 | 1.10 | 92.91 | 0.00 | 0.00 | 0.91 | 22.446 | 28.714 |
| pH 5; S5204; T720; D2270-33 | 0.40 | 6.95 | 1.76 | 89.89 | 0.00 | 0.00 | 2.28 | 22.408 | 29.656 |
| pH 5; S5204; T718; D2263-14 | 0.58 | 7.72 | 1.64 | 89.26 | 0.00 | 0.00 | 0.306 | 22.35 | 32.294 |
| pH 5; S5204; T720; D2270-34 | 0.36 | 6.75 | 1.77 | 90.10 | 0.00 | 0.00 | 2.398 | 22.3 | 28.958 |
| pH 7; S5204; T672; D2090-29 | 0.42 | 4.99 | 2.01 | 91.06 | 0.00 | 0.00 | 1.16 | 22.112 | 30.376 |
| pH 5; S5204; T720; D2269-14 | 0.00 | 7.86 | 1.80 | 89.57 | 0.00 | 0.00 | 0.574 | 21.802 | 31.558 |
| pH 5; S5204; T718; D2263-29 | 0.58 | 7.32 | 1.30 | 90.07 | 0.00 | 0.00 | 0.418 | 21.746 | 30.426 |
| pH 5; S5204; T718; D2263-19 | 0.62 | 7.92 | 1.56 | 89.25 | 0.00 | 0.00 | 0.574 | 21.692 | 29.514 |
| pH 5; S5204; T720; D2269-10 | 0.39 | 6.82 | 1.70 | 90.05 | 0.00 | 0.00 | 1.104 | 21.622 | 25.264 |
| pH 5; S5204; T720; D2269-4 | 0.42 | 6.57 | 1.71 | 90.32 | 0.00 | 0.00 | 1.082 | 21.466 | 29.698 |
| pH 5; S5204; T720; D2270-4 | 0.43 | 7.44 | 1.72 | 89.31 | 0.00 | 0.00 | 1.758 | 21.446 | 32.656 |
| pH 5; S5204; T720; D2269-34 | 0.00 | 6.69 | 1.78 | 90.64 | 0.00 | 0.00 | 0.946 | 21.438 | 28.538 |
| pH 5; S5204; T720; D2270-16 | 0.39 | 7.08 | 1.71 | 89.70 | 0.00 | 0.00 | 1.592 | 21.422 | 27.72 |
| pH 5; S5204; T718; D2263-26 | 0.42 | 7.39 | 1.70 | 89.28 | 0.00 | 0.00 | 0.514 | 21.328 | 29.746 |
| pH 5; S5204; T720; D2269-3 | 0.36 | 6.76 | 1.71 | 90.17 | 0.00 | 0.00 | 0.668 | 21.242 | 29.74 |
| pH 5; S5204; T720; D2270-22 | 0.35 | 6.77 | 1.67 | 90.15 | 0.00 | 0.00 | 1.194 | 21.026 | 25.084 |
| pH 5; S5204; T720; D2270-26 | 0.41 | 6.81 | 1.82 | 89.66 | 0.00 | 0.00 | 1.606 | 20.948 | 32.142 |
| pH 5; S5204; T720; D2270-10 | 0.46 | 6.98 | 1.80 | 90.03 | 0.00 | 0.00 | 0.792 | 20.728 | 28.264 |
| pH 5; S5204; T720; D2269-16 | 0.51 | 6.17 | 1.50 | 90.64 | 0.00 | 0.00 | 0.922 | 20.502 | 30.132 |
| pH 5; S5204; T720; D2270-8 | 0.50 | 6.95 | 1.42 | 90.34 | 0.00 | 0.00 | 2.252 | 20.486 | 28.34 |
| pH 5; S5204; T720; D2270-2 | 0.46 | 6.76 | 1.83 | 89.90 | 0.00 | 0.00 | 0.97 | 20.366 | 31.758 |
| pH 5; S5204; T720; D2269-36 | 0.00 | 7.43 | 1.66 | 89.88 | 0.00 | 0.00 | 0.754 | 20.006 | 29.648 |
| pH 5; S5204; T720; D2269-31 | 0.72 | 9.29 | 1.86 | 86.92 | 0.00 | 0.00 | 2.062 | 19.002 | 27.61 |
| pH 5; S5204; T720; D2269-44 | 0.00 | 9.45 | 1.58 | 88.16 | 0.00 | 0.00 | 1.378 | 18.576 | 22.52 |
| pH 7; S5204; T672; D2091-14 | 0.27 | 4.79 | 2.24 | 90.94 | 0.00 | 0.00 | 0.93 | 18.1 | 30.434 |
| pH 5; S5204; T720; D2270-32 | 0.40 | 7.14 | 1.74 | 89.63 | 0.00 | 0.00 | 1.668 | 17.966 | 27.06 |
| pH 5; S5204; T720; D2270-11 | 0.82 | 9.24 | 1.93 | 87.35 | 0.00 | 0.00 | 1.178 | 15.998 | 28.196 |
| pH 5; S5204; T720; D2269-48 | 0.72 | 9.05 | 2.14 | 88.08 | 0.00 | 0.00 | 1.172 | 14.694 | 25.384 |
| pH 5; S5204; T720; D2269-17 | 0.66 | 9.08 | 2.12 | 87.12 | 0.00 | 0.00 | 0.84 | 14.488 | 25.886 |
| pH 5; S5204; T720; D2270-20 | 0.62 | 8.35 | 1.97 | 88.43 | 0.00 | 0.00 | 1.37 | 14.168 | 23.794 |
| pH 5; S5204; T718; D2263-13 | 0.75 | 9.44 | 1.98 | 87.09 | 0.00 | 0.00 | 0.64 | 13.854 | 29.466 |
| pH 5; S5204; T720; D2269-46 | 0.43 | 6.87 | 1.71 | 89.81 | 0.00 | 0.00 | 0.646 | 10.452 | 31.464 |
| pH 5; S5204; T720; D2269-5 | 0.59 | 8.81 | 1.93 | 87.97 | 0.00 | 0.00 | 0.654 | 9.37 | 25.786 |
| pH 7; S5204; T672; D2091-4 | 1.42 | 4.39 | 2.32 | 89.87 | 0.00 | 0.00 | 0.686 | 8.182 | 16.454 |
| pH 5; S5204; T720; D2269-6 | 0.50 | 7.29 | 1.73 | 89.29 | 0.00 | 0.00 | 0.79 | 7.978 | 21.346 |
| pH 5; S5204; T720; D2270-45 | 0.00 | 9.16 | 1.65 | 88.19 | 0.00 | 0.00 | 0.464 | 3.448 | 16.796 |
| Blank | | | | | | | 0 | 0 | 0 |

It is comtemplated that these promoters, or variants thereof, discovered here can be used to regulate a fatty acid synthesis gene (e.g., any of the FATA, FATB, SAD, FAD2, KASI/IV, KASII, LPAAT or KCS genes disclosed herein) or other gene or gene-suppression element expressed in a cell including a microalgal cell. Variants can have for example 60, 65, 70, 75, 80, 85, 90, 95, 96, 97, 98, 99% or greater identity to the sequences disclosed here.

### Example 8

### Combining KASII, FATA and LPAAT Transgenes to Produce an Oil High in SOS

In Prototheca moriformis, we overexpressed the P. moriformis KASII, knocked out an endogenous SAD2 allele, knocked out the endogenous FATA allele, and overexpressed both a LPAAT from Brassica napus and a FATA gene from Garcinia mangostana (“GarmFAT1”). The resulting strain produced an oil with over 55% SOS, over 70% Sat-O-Sat, and less than 8% trisaturated TAGs.

A base strain was transformed with a linearized plasmid with flanking regions designed for homologous recombination at the SAD2 site. The construct ablated SAD2 and overexpressed P. moriformis KASII. A ThiC selection marker was used. This strain was further transformed with a construct designed to overexpress GarmFATA1 with a P. moriformis SASD1 plastid targeting peptide via homologous recombination at the 6S chromosomal site using invertase as a selection marker. The resulting strain, produced oil with about 62% stearate, 6% palmitate, 5% linoleate, 45% SOS and 20% trisaturates.

The sequence of the transforming DNA from the GarmFATA1 expression construct (pSZ3204) is shown below in SEQ ID NO:61. Relevant restriction sites are indicated in lowercase, bold, and are from 5′-3′ BspQI, KpnI, XbaI, MfeI, BamHI, AvrII, EcoRV, SpeI, AscI, ClaI, AflII, SacI and BspQI. Underlined sequences at the 5′ and 3′ flanks of the construct represent genomic DNA from P. moriformis that enable targeted integration of the transforming DNA via homologous recombination at the 6S locus. Proceeding in the 5′ to 3′ direction, the CrTUB2 promoter driving the expression of Saccharomyces cerevisiae SUC2 (ScSUC2) gene, enabling strains to utilize exogenous sucrose, is indicated by lowercase, boxed text. The initiator ATG and terminator TGA of ScSUC2 are indicated by uppercase italics, while the coding region is represented by lowercase italics. The 3′ UTR of the CvNR gene is indicated by small capitals. A spacer region is represented by lowercase text. The P. moriformis SAD2-2 (PmSAD2-2) promoter driving the expression of the chimeric CpSAD1tp_GarmFATA1_FLAG gene is indicated by lowercase, boxed text. The initiator ATG and terminator TGA are indicated by uppercase italics; the sequence encoding CpSAD1tp is represented by lowercase, underlined italics; the sequence encoding the GarmFATA1 mature polypeptide is indicated by lowercase italics; and the 3× FLAG epitope tag is represented by uppercase, bold italics. A second CvNR 3′ UTR is indicated by small capitals.

Nucleotide sequence of the transforming DNA from pSZ3204:
(SEQ ID NO:61)
gctcttcGCCGCCGCCACTCCTGCTCGAGCGCGCCCGCGCGTGCGCCGCCAGCGCCTTGGCCTTTTCGC
CGCGCTCGTGCGCGTCGCTGATGTCCATCACCAGGTCCATGAGGTCTGCCTTGCGCCGGCTGAGCCA
CTGCTTCGTCCGGGCGGCCAAGAGGAGCATGAGGGAGGACTCCTGGTCCAGGGTCCTGACGTGGT
CGCGGCTCTGGGAGCGGGCCAGCATCATCTGGCTCTGCCGCACCGAGGCCGCCTCCAACTGGTCCT
CCAGCAGCCGCAGTCGCCGCCGACCCTGGCAGAGGAAGACAGGTGAGGGGGGTATGAATTGTACA
GAACAACCACGAGCCTTGTCTAGGCAGAATCCCTACCAGTCATGGCTTTACCTGGATGACGGCCTGC
GAACAGCTGTCCAGCGACCCTCGCTGCCGCCGCTTCTCCCGCACGCTTCTTTCCAGCACCGTGATGGC
GCGAGCCAGCGCCGCACGCTGGCGCTGCGCTTCGCCGATCTGAGGACAGTCGGGGAACTCTGATCA
GTCTAAACCCCCTTGCGCGTTAGTGTTGCCATCCTTTGCAGACCGGTGAGAGCCGACTTGTTGTGCG
CCACCCCCCACACCACCTCCTCCCAGACCAATTCTGTCACCTTTTTGGCGAAGGCATCGGCCTCGGCC

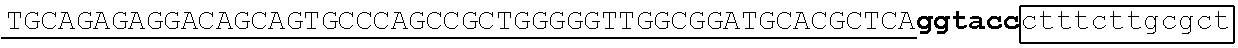

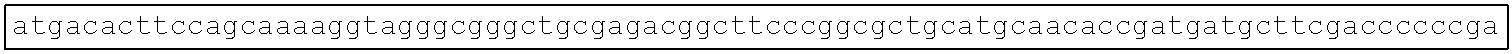

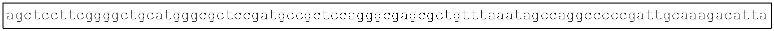

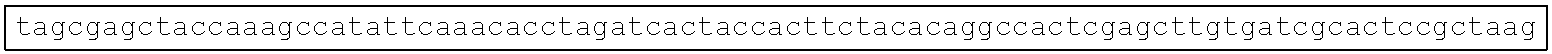

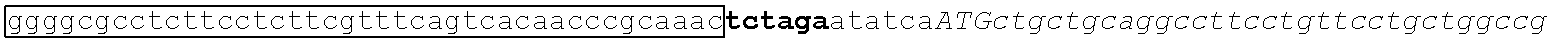

gcttcgccgccaagatcagcgcctccatgacgaacgagacgtccgaccgccccctggtgcacttcacccccaacaagggctgg
atgaacgaccccaacggcctgtggtacgacgagaaggacgccaagtggcacctgtacttccagtacaacccgaacgacacc
gtctgggggacgcccttgttctggggccacgccacgtccgacgacctgaccaactgggaggaccagcccatcgccatcgcccc
gaagcgcaacgactccggcgccttctccggctccatggtggtggactacaacaacacctccggcttcttcaacgacaccatcga
cccgcgccagcgctgcgtggccatctggacctacaacaccccggagtccgaggagcagtacatctcctacagcctggacggcg
gctacaccttcaccgagtaccagaagaaccccgtgctggccgccaactccacccagttccgcgacccgaaggtcttctggtacg
agccctcccagaagtggatcatgaccgcggccaagtcccaggactacaagatcgagatctactcctccgacgacctgaagtcc
tggaagctggagtccgcgttcgccaacgagggcttcctcggctaccagtacgagtgccccggcctgatcgaggtccccaccga
gcaggaccccagcaagtcctactgggtgatgttcatctccatcaaccccggcgccccggccggcggctccttcaaccagtacttc
gtcggcagcttcaacggcacccacttcgaggccttcgacaaccagtcccgcgtggtggacttcggcaaggactactacgccctg
cagaccttcttcaacaccgacccgacctacgggagcgccctgggcatcgcgtgggcctccaactgggagtactccgccttcgtg
cccaccaacccctggcgctcctccatgtccctcgtgcgcaagttctccctcaacaccgagtaccggccaacccggagacggag
ctgatcaacctgaaggccgagccgatcctgaacatcagcaacgccggcccctggagccggttcgccaccaacaccacgttgac
gaaggccaacagctacaacgtcgacctgtccaacagcaccggcaccctggagttcgagctggtgtacgccgtcaacaccacc
cagacgatctccaagtccgtgttcgcggacctctccctctggttcaagggcctggaggaccccgaggagtacctccgcatgggc
ttcgaggtgtccgcgtcctccttcttcctggaccgcgggaacagcaaggtgaagttcgtgaaggagaacccctacttcaccaac
cgcatgagcgtgaacaaccagcccttcaagagcgagaacgacctgtcctactacaaggtgtacggcttgctggaccagaaca
tcctggagctgtacttcaacgacggcgacgtcgtgtccaccaacacctacttcatgaccaccgggacgccctgggctccgtga
acatgacgacgggggtggacaacctgttctacatcgacaagttccaggtgcgcgaggtcaagTGAcaattgGCAGCAGCAG
CTCGGATAGTATCGACACACTCTGGACGCTGGTCGTGTGATGGACTGTTGCCGCCACACTTGCTGCCTTGACCTGTGAATA
TCCCTGCCGCTTTTATCAAACAGCCTCAGTGTGTTTGATCTTGTGTGTACGCGCTTTTGCGAGTTGCTAGCTGCTTGTGCTA
TTTGCGAATACCACCCCCAGCATCCCCTTCCCTCGTTTCATATCGCTTGCATCCCAACCGCAACTTATCTACGCTGTCCTGCT
ATCCCTCAGCGCTGCTCCTGCTCCTGCTCACTGCCCCTCGCACAGCCTTGGTTTGGGCTCCGCCTGTATTCTCCTGGTACTG
CAACCTGTAAACCAGCACTGCAATGCTGATGCACGGGAAGTAGTGGGATGGGAACACAAATGGAggatcccgcgtctcga
acagagcgcgcagaggaacgctgaaggtctcgcctctgtcgcacctcagcgcggcatacaccacaataaccacctgacgaatgcg
cttggttcttcgtccattagcgaagcgtccggttcacacacgtgccacgttggcgaggtggcaggtgacaatgatcggtggagctgat

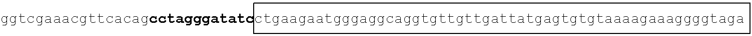

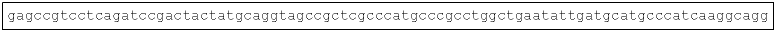

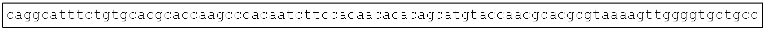

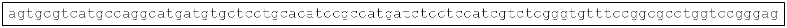

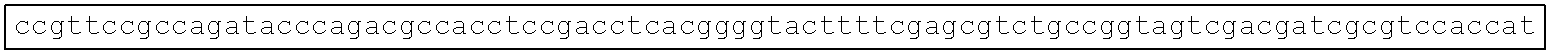

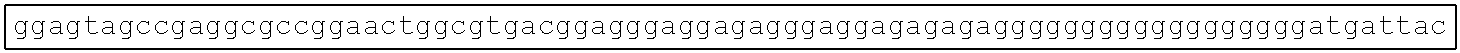

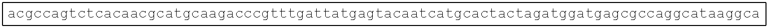

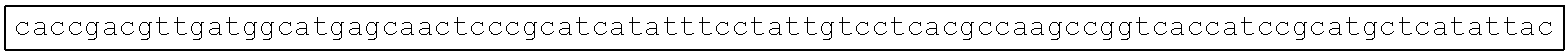

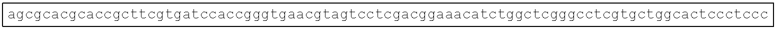

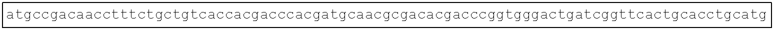

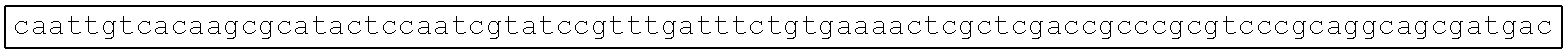

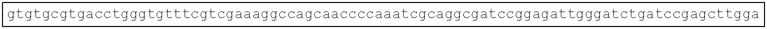

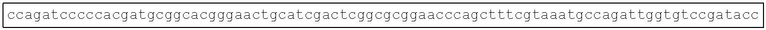

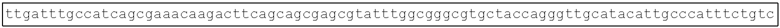

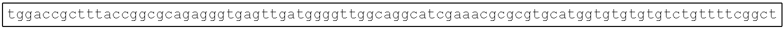

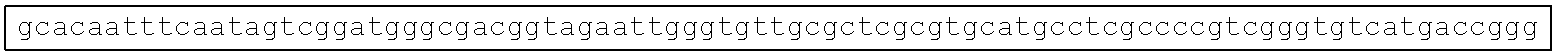

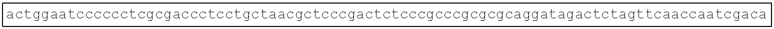

actagtATGgccaccgcatccactttctcggcgttcaatgcccgctgcggcgacctgcgtcgctcggcgggctccgggccccgg
cgcccagcgaggcccctccccgtgcgcgggcgcgccatccccccccgcatcatcgtggtgtcctcctcctcctccaaggtgaaccc
cctgaagaccgaggccgtggtgtcctccggcctggccgaccgcctgcgcctgggctccctgaccgaggacggcctgtcctaca
aggagaagttcatcgtgcgctgctacgaggtgggcatcaacaagaccgccaccgtggagaccatcgccaacctgctgcagg
aggtgggctgcaaccacgcccagtccgtgggctactccaccggcggcttctccaccacccccaccatgcgcaagctgcgcctga
tctgggtgaccgcccgcatgcacatcgagatctacaagtaccccgcctggtccgacgtggtggagatcgagtcctggggccag
ggcgagggcaagatcggcacccgccgcgactggatcctgcgcgactacgccaccggccaggtgatcggccgcgccacctcca
agtgggtgatgatgaaccaggacacccgccgcctgcagaaggtggacgtggacgtgcgcgacgagtacctggtgcactgcc
cccgcgagctgcgcctggccttccccgaggagaacaactcctccctgaagaagatctccaagctggaggacccctcccagtac
tccaagctgggcctggtgccccgccgcgccgacctggacatgaaccagcacgtgaacaacgtgacctacatcggctgggtgct
ggagtccatgccccaggagatcatcgacacccacgagctgcagaccatcaccctggactaccgccgcgagtgccagcacgac
gacgtggtggactccctgacctcccccgagccctccgaggacgccgaggccgtgttcaaccacaacggcaccaacggctccgc
caacgtgtccgccaacgaccacggctgccgcaacttcctgcacctgctgcgcctgtccggcaacggcctggagatcaaccgcg
gccgcaccgagtggcgcaagaagcccacccgcATGGACTACAAGGACCACGACGGCGACTACAAGGACCAC
GACATCGACTACAAGGACGACGACGACAAGTGAatcgatagatctcttaagGCAGCAGCAGCTCGGATAGTAT
CGACACACTCTGGACGCTGGTCGTGTGATGGACTGTTGCCGCCACACTTGCTGCCTTGACCTGTGAATATCCCTGCCGCTT
TTATCAAACAGCCTCAGTGTGTTTGATCTTGTGTGTACGCGCTTTTGCGAGTTGCTAGCTGCTTGTGCTATTTGCGAATACC
ACCCCCAGCATCCCCTTCCCTCGTTTCATATCGCTTGCATCCCAACCGCAACTTATCTACGCTGTCCTGCTATCCCTCAGCGC
TGCTCCTGCTCCTGCTCACTGCCCCTCGCACAGCCTTGGTTTGGGCTCCGCCTGTATTCTCCTGGTACTGCAACCTGTAAAC
CAGCACTGCAATGCTGATGCACGGGAAGTAGTGGGATGGGAACACAAATGGAaagcttaattaagagctcTTGTTTTCC
AGAAGGAGTTGCTCCTTGAGCCTTTCATTCTCAGCCTCGATAACCTCCAAAGCCGCTCTAATTGTGGA
GGGGGTTCGAATTTAAAAGCTTGGAATGTTGGTTCGTGCGTCTGGAACAAGCCCAGACTTGTTGCTC
ACTGGGAAAAGGACCATCAGCTCCAAAAAACTTGCCGCTCAAACCGCGTACCTCTGCTTTCGCGCAA
TCTGCCCTGTTGAAATCGCCACCACATTCATATTGTGACGCTTGAGCAGTCTGTAATTGCCTCAGAAT
GTGGAATCATCTGCCCCCTGTGCGAGCCCATGCCAGGCATGTCGCGGGCGAGGACACCCGCCACTC
GTACAGCAGACCATTATGCTACCTCACAATAGTTCATAACAGTGACCATATTTCTCGAAGCTCCCCAA
CGAGCACCTCCATGCTCTGAGTGGCCACCCCCCGGCCCTGGTGCTTGCGGAGGGCAGGTCAACCGG
CATGGGGCTACCGAAATCCCCGACCGGATCCCACCACCCCCGCGATGGGAAGAATCTCTCCCCGGG
ATGTGGGCCCACCACCAGCACAACCTGCTGGCCCAGGCGAGCGTCAAACCATACCACACAAATATCC
TTGGCATCGGCCCTGAATTCCTTCTGCCGCTCTGCTACCCGGTGCTTCTGTCCGAAGCAGGGGTTGCT
AGGGATCGCTCCGAGTCCGCAAACCCTTGTCGCGTGGCGGGGCTTGTTCGAGCTTgaagagc

The resulting strain was further transformed with a construct designed to recombine at (and thereby disrupt) the endogenous FATA and also express the LPAAT from B. napus under control of the UAPA1 promoter and using alpha galactosidase as a selectable marker with selection on melbiose. The resulting strain showed increased production of SOS (about 57-60%) and Sat-O-Sat (about 70-76%) and lower amounts of trisaturates (4.8 to 7.6%).

Strains were generated in the high-C18:0 56573 background in which we maximized SOS production and minimized the formation of trisaturated TAGs by targeting both the Brassica napus LPAT2(Bn1.13) gene and the PmFAD2hpA RNAi construct to the FATA-1 locus. The sequence of the transforming DNA from the PmFAD2hpA expression construct pSZ4164 is shown below in SEQ ID NO:62. Relevant restriction sites are indicated in lowercase, bold, and are from 5′-3′ BspQI, KpnI, SpeI, SnaBI, BamHI, NdeI, NsiI, AflII, EcoRI, SpeI, BsiWI, XhoI, SacI and BspQI. Underlined sequences at the 5′ and 3′ flanks of the construct represent genomic DNA from P. moriformis that enable targeted integration of the transforming DNA via homologous recombination at the FATA-1 locus. Proceeding in the 5′ to 3′ direction, the PmHXT1 promoter driving the expression of Saccharomyces carlbergensis MEL1 (ScarMEL1) gene, enabling strains to utilize exogenous melibiose, is indicated by lowercase, boxed text. The initiator ATG and terminator TGA of ScarMEL1 are indicated by uppercase italics, while the coding region is represented by lowercase italics. The 3′ UTR of the P. moriformis PGK gene is indicated by small capitals. A spacer region is represented by lowercase text. The P. moriformis UAPA1 promoter driving the expression of the BnLPAT2(Bn1.13) gene is indicated by lowercase, boxed text. The initiator ATG and terminator TGA are indicated by uppercase italics; the sequence encoding BnLPAT2(Bn1.13) is represented by lowercase, underlined italics. The 3′ UTR of the CvNR gene is indicated by small capitals. A second spacer region is represented by lowercase text. The C. reinhardtii CrTUB2 promoter driving the expression of the PmFAD2hpA hairpin sequence is indicated by lowercase, boxed text. The FAD2 exon 1 sequence in the forward orientation is indicated with lowercase italics; the FAD2 intron 1 sequence is represented with lowercase, bold italics; a short linker region is indicated with lowercase text, and the FAD2 exon 1 sequence in the reverse orientation is indicated with lowercase, underlined italics. A second CvNR 3′ UTR is indicated by small capitals.

Nucleotide sequence of the transforming DNA from pSZ4164:
(SEQ ID NO:62)
gctcttcCCAACTCAGATAATACCAATACCCCTCCTTCTCCTCCTCATCCATTCAGTACCCCCCCCCTTCTC
TTCCCAAAGCAGCAAGCGCGTGGCTTACAGAAGAACAATCGGCTTCCGCCAAAGTCGCCGAGCACT
GCCCGACGGCGGCGCGCCCAGCAGCCCGCTTGGCCACACAGGCAACGAATACATTCAATAGGGGG
CCTCGCAGAATGGAAGGAGCGGTAAAGGGTACAGGAGCACTGCGCACAAGGGGCCTGTGCAGGA
GTGACTGACTGGGCGGGCAGACGGCGCACCGCGGGCGCAGGCAAGCAGGGAAGATTGAAGCGGC
AGGGAGGAGGATGCTGATTGAGGGGGGCATCGCAGTCTCTCTTGGACCCGGGATAAGGAAGCAAA
TATTCGGCCGGTTGGGTTGTGTGTGTGCACGTTTTCTTCTTCAGAGTCGTGGGTGTGCTTCCAGGGA
GGATATAAGCAGCAGGATCGAATCCCGCGACCAGCGTTTCCCCATCCAGCCAACCACCCTGTCggtac

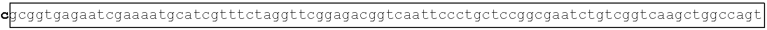

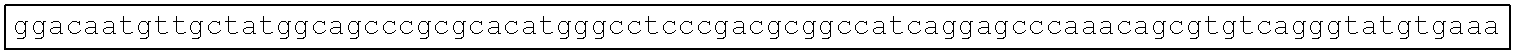

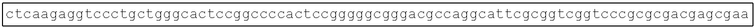

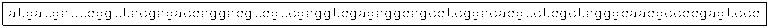

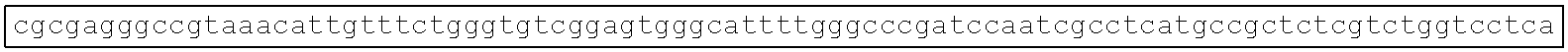

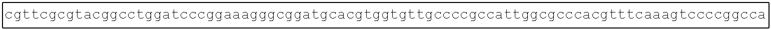

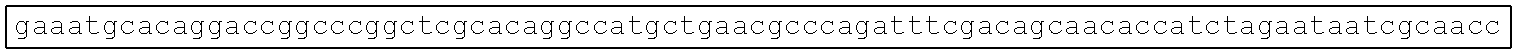

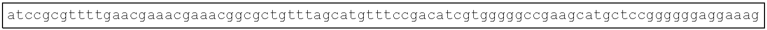

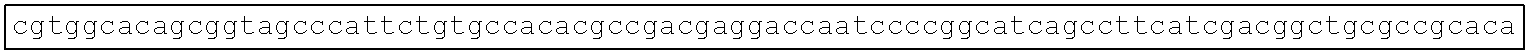

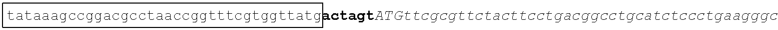

gtgttcggcgtctccccctcctacaacggcctgggcctgacgccccagatgggctgggacaactggaacacgttcgcctgcgac
gtctccgagcagctgctgctggacacggccgaccgcatctccgacctgggcctgaaggacatgggctacaagtacatcatcct
ggacgactgctggtcctccggccgcgactccgacggcttcctggtcgccgacgagcagaagttccccaacggcatgggccacg
tcgccgaccacctgcacaacaactccttcctgttcggcatgtactcctccgcgggcgagtacacgtgcgccggctaccccggctc
cctgggccgcgaggaggaggacgcccagttcttcgcgaacaaccgcgtggactacctgaagtacgacaactgctacaacaa
gggccagttcggcacgcccgagatctcctaccaccgctacaaggccatgtccgacgccctgaacaagacgggccgccccatct
tctactccctgtgcaactggggccaggacctgaccttctactggggctccggcatcgcgaaactcctggcgcatgtccggcgacgt
cacggcggagttcacgcgccccgactcccgctgcccctgcgacggcgacgagtacgactgcaagtacgccggcttccactgctc
catcatgaacatcctgaacaaggccgcccccatgggccagaacgcgggcgtcggcggctggaacgacctggacaacctgga
ggtcggcgtcggcaacctgacggacgacgaggagaaggcgcacttctccatgtgggccatggtgaagtcccccctgatcatc
ggcgcgaacgtgaacaacctgaaggcctcctcctactccatctactcccaggcgtccgtcatcgccatcaaccaggactccaac
ggcatccccgccacgcgcgtctggcgctactacgtgtccgacacggacgagtacggccagggcgagatccagatgtggtccg
gccccctggacaacggcgaccaggtcgtggcgctgctgaacggcggctccgtgtcccgccccatgaacacgaccctggagga
gatcttcttcgactccaacctgggctccaagaagctgacctccacctgggacatctacgacctgtgggcgaaccgcgtcgacaa
ctccacggcgtccgccatcctgggccgcaacaagaccgccaccggcatcctgtacaacgccaccgagcagtcctacaaggac
ggcctgtccaagaacgacacccgcctgttcggccagaagatcggctccctgtcccccaacgcgatcctgaacacgaccgtcccc
gcccacggcatcgcgttctaccgcctgcgcccctcctccTGAacaacttattacgtaTTCTGACCGGCGCTGATGTGGCGCGG
ACGCCGTCGTACTCTTTCAGACTTTACTCTTGAGGAATTGAACCTTTCTCGCTTGCTGGCATGTAAACATTGGCGCAATTAA
TTGTGTGATGAAGAAAGGGTGGCACAAGATGGATCGCGAATGTACGAGATCGACAACGATGGTGATTGTTATGAGGGG
CCAAACCTGGCTCAATCTTGTCGCATGTCCGGCGCAATGTGATCCAGCGGCGTGACTCTCGCAACCTGGTAGTGTGTGCG
CACCGGGTCGCTTTGATTAAAACTGATCGCATTGCCATCCCGTCAACTCACAAGCCTACTCTAGCTCCCATTGCGCACTCGG
GCGCCCGGCTCGATCAATGTTCTGAGCGGAGGGCGAAGCGTCAGGAAATCGTCTCGGCAGCTGGAAGCGCATGGAATGC
GGAGCGGAGATCGAATCAggatcccgcgtctcgaacagagcgcgcagaggaacgctgaaggtctcgcctctgtcgcacctcagc
gcggcatacaccacaataaccacctgacgaatgcgcttggttcttcgtccattagcgaagcgtccggttcacacacgtgccacgttg

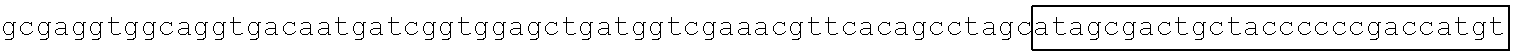

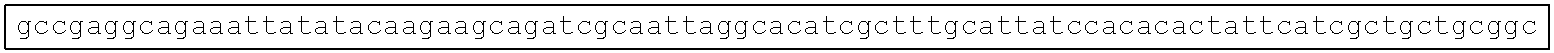

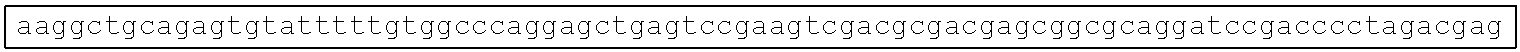

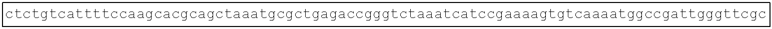

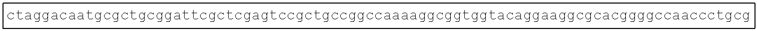

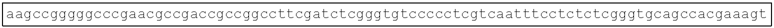

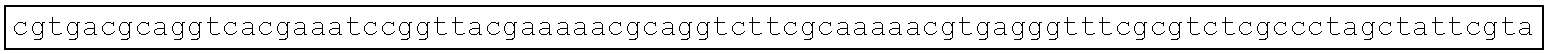

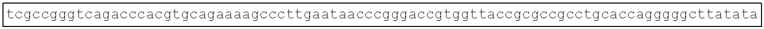

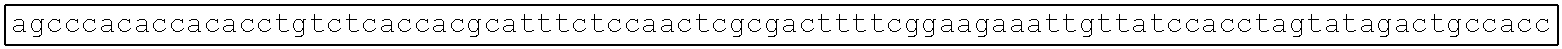

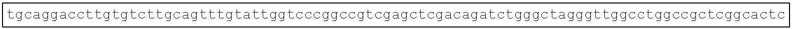

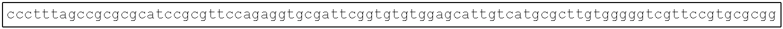

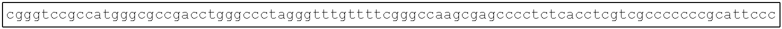

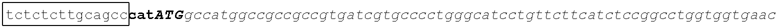

ctgctgcaggccatctgctacgtgctgatccgccccctgtccaagaacacctaccgcaagatcaaccgcgtggtggccgagacc
ctgtggctggagctggtgtggatcgtggactggtgggccggcgtgaagatccaggtgttcgccgacaacgagaccttcaacc
gcatgggcaaggagcacgccctggtggtgtgcaaccaccgctccgacatcgactggctggtgggctggatcctggcccagcg
ctccggctgcctgggctccgccctggccgtgatgaagaagtcctccaagttcctgcccgtgatcggctggtccatgtggttctccg
agtacctgacctggagcgcaactgggccaaggacgagtccaccctgaagtccggcctgcagcgcctgaacgacttcccccgc
cccttctggctggccctgttcgtggagggcacccgcttcaccgaggccaagctgaaggccgcccaggagtacgccgcctcctcc
gagctgcccgtgccccgcaacgtgctgatcccccgcaccaagggcttcgtgtccgccgtgtccaacatgcgctccttcgtgcccg
ccatctacgacatgaccgtggccatccccaagacctcccccccccccaccatgctgcgcctgttcaagggccagccctccgtggt
gcacgtgcacatcaagtgccactccatgaaggacctgcccgagtccgacgacgccatcgcccagtggtgccgcgaccagttcg
tggccaaggacgccctgctggacaagcacatcgccgccgacaccttccccggccagcaggagcagaacatcggccgccccat
caagtccctggccgtggtgctgtcctggtcctgcctgctgatcctgggcgccatgaagttcctgcactggtccaacctgactcctc
ctggaagggcatcgccactccgccctgggcctgggcatcatcaccctgtgcatgcagatcctgatccgctcctcccagtccgag
cgctccacccccgccaaggtggtgcccgccaagcccaaggacaaccacaacgactccggctcctcctcccagaccgaggtgga
gaagcagaagTGAatgcatGCAGCAGCAGCTCGGATAGTATCGACACACTCTGGACGCTGGTCGTGTGATGGACTGTTG
CCGCCACACTTGCTGCCTTGACCTGTGAATATCCCTGCCGCTTTTATCAAACAGCCTCAGTGTGTTTGATCTTGTGTGTACG
CGCTTTTGCGAGTTGCTAG CTG CTTGTGCTATTTGCGAATACCACCCCCAGCATCCCCTTCCCTCGTTTCATATCGCTTGCAT
CCCAACCGCAACTTATCTACGCTGTCCTGCTATCCCTCAGCGCTGCTCCTGCTCCTGCTCACTGCCCCTCGCACAGCCTTGG
TTTGGGCTCCGCCTGTATTCTCCTGGTACTGCAACCTGTAAACCAGCACTGCAATGCTGATGCACGGGAAGTAGTGGGAT
GGGAACACAAATGGActtaaggatctaagtaagattcgaagcgctcgaccgtgccggacggactgcagccccatgtcgtagtga
ccgccaatgtaagtgggctggcgtttccctgtacgtgagtcaacgtcactgcacgcgcaccaccctctcgaccggcaggaccaggca
tcgcgagatacagcgcgagccagacacggagtgccgagctatgcgcacgctccaactagatatcatgtggatgatgagcatgaatt

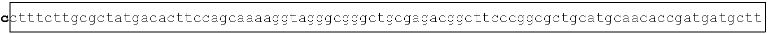

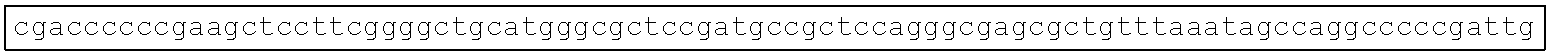

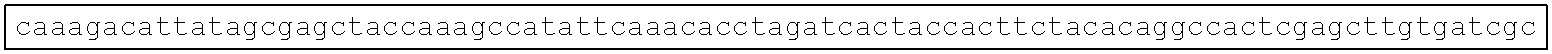

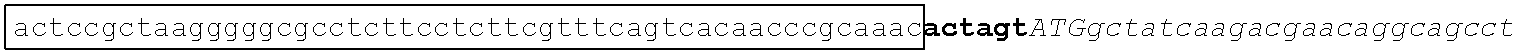

gtggagaagcctccgttcacgatcgggacgctgcgcaaggccatccccgcgcactgtacgagcgctcggcgcttcgtagcag
catgtacctggcctttgacatcgcggtcatgtccctgctctacgtcgcgtcgacgtacatcgaccctgcaccggtgcctacgtggg

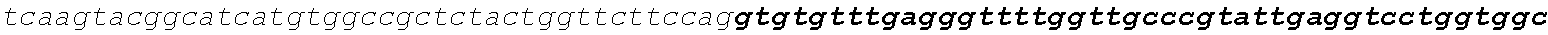

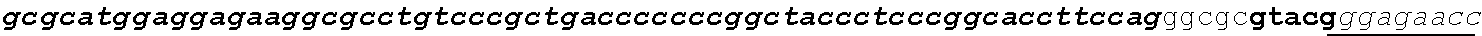

agtagagcagccacatgatqccgtacttgacccacgtaggcaccgatqcaggatcgatatacgtcgacgcgacgtagagca
gggacatgaccgcgatgtcaaaggccaggtacatgctgctacgaagcgccgagcgctcgaaacagtgcgcggggatggcct
tgcgcagcgtcccgatcgtgaacggaggcttctccacaggctgcctgttcgtcttgatagccatctcgagGCAGCAGCAGCTCG
GATAGTATCGACACACTCTGGACGCTGGTCGTGTGATGGACTGTTGCCGCCACACTTGCTGCCTTGACCTGTGAATATCCC
TGCCGCTTTTATCAAACAGCCTCAGTGTGTTTGATCTTGTGTGTACGCGCTTTTGCGAGTTGCTAGCTGCTTGTGCTATTTG
CGAATACCACCCCCAGCATCCCCTTCCCTCGTTTCATATCGCTTGCATCCCAACCGCAACTTATCTACGCTGTCCTGCTATCC
CTCAGCGCTGCTCCTGCTCCTGCTCACTGCCCCTCGCACAGCCTTGGTTTGGGCTCCGCCTGTATTCTCCTGGTACTGCAAC
CTGTAAACCAGCACTGCAATGCTGATGCACGGGAAGTAGTGGGATGGGAACACAAATGGAAAGCTGTAgagctcttgtttt
ccagaaggagttgctccttgagcctttcattctcagcctcgataacctccaaagccgctctaattgtggagggggttcgaaCCGAA
TGCTGCGTGAACGGGAAGGAGGAGGAGAAAGAGTGAGCAGGGAGGGATTCAGAAATGAGAAATG
AGAGGTGAAGGAACGCATCCCTATGCCCTTGCAATGGACAGTGTTTCTGGCCACCGCCACCAAGACT
TCGTGTCCTCTGATCATCATGCGATTGATTACGTTGAATGCGACGGCCGGTCAGCCCCGGACCTCCA
CGCACCGGTGCTCCTCCAGGAAGATGCGCTTGTCCTCCGCCATCTTGCAGGGCTCAAGCTGCTCCCA
AAACTCTTGGGCGGGTTCCGGACGGACGGCTACCGCGGGTGCGGCCCTGACCGCCACTGTTCGGAA
GCAGCGGCGCTGCATGGGCAGCGGCCGCTGCGGTGCGCCACGGACCGCATGATCCACCGGAAAAG
CGCACGCGCTGGAGCGCGCAGAGGACCACAGAGAAGCGGAAGAGACGCCAGTACTGGCAAGCAG
GCTGGTCGGTGCCATGGCGCGCTACTACCCTCGCTATGACTCGGGTCCTCGGCCGGCTGGCGGTGCT
GACAATTCGTTTAGTGGAGCAGCGACTCCATTCAGCTACCAGTCGAACTCAGTGGCACAGTGACTcc
gctcttc

### Example 9

### Algal Oil with “Zero” Saturated Fat Per Serving

In this example, we demonstrate that triacylglycerols in Prototheca moriformis (derived from UTEX 1435) can be significantly reduced in levels of saturated fatty acids, utilizing both molecular genetics and classical mutagenesis approaches. As described below, strain S8188 produces oil with less than or about 3% total saturated fatty acids in multiple fermentation runs. Strain 8188 expresses exogenous genes that produce the mature KASII and SAD proteins of SEQ ID NOS: 64 and 65, respectively with an insertion that disrupts the expression of an endogenous FATA allele.

Summary of Strain S8188 Generation.

The strain S8188 was created by two successive transformations. The high oleic base strain S7505 was first transformed with pSZ3870 (FATA1 3′::CrTUB2-ScSUC2-CvNR:PmSAD2-2-CpSADtp-PmKASII-CvNR::FATA1 5′), a construct that disrupts a single copy of the FATA1 allele while simultaneously overexpressing the P. moriformis KASII. The resulting high-oleic, lower-palmitic strain S7740 produces 1.4% palmitate with 7.3% total saturates in fermentation runs (Table 52).

Specifically, S7505 and S5100 are cerulenen resistant isolates of Strain S3150 with low C16:0 titer and high C18:1 titer made according to the methods disclosed in co-owned application 62/141,167 filed on 31 Mar. 2015.

S7740 was subsequently transformed with pSZ4768 (FAD2-1 5′::PmHXT1V2-ScarMEL1-PmPGK:PmSAD2-2p-CpSADtp-PmKASII-CvNR:PmACP1-PmSAD2-1-CvNR::FAD2-1 3′), introducing another copy of PmKASII and simultaneously overexpressing PmSAD2-1 gene targeting the FAD2 (delta-12 fatty acid desaturase) locus, to yield strain S8188. Strain S8188 produces 1.7% C16:0 and 0.5% C18:0, and total saturated fatty acids levels around 3% (Table 52). Note that disrupting FAD2 elevates the levels of oleic acid relative to polyunsaturates, but this disruption may not be needed to achieve low levels of unsaturates.

TABLE 52
Comparison of fatty acid profiles between strains S7505, S7740 and
S8188 in high cell-density fermentation experiment. Strain S7740
produces lower C16:0; while S8188 produces lower C16:0 and C18:0,
therefore lower in total saturated fatty acids.

| | Fatty Acids Area % | | | | |
|---|---|---|---|---|---|
| Strains | C16:0 | C18:0 | C18:1 | C18:2 | Total saturates % |
| S7505 | 12.5 | 5.6 | 75.5 | 4.8 | 18.9 |
| S7740 | 1.4 | 4.9 | 85.2 | 5.1 | 7.3 |
| S8188 | 1.7 | 0.5 | 91.8 | 3.8 | 3.0 |

Optimization of PmKASII Expression to Generate a Lower Palmitic Strain.

The major saturated fatty acids in P. moriformis UTEX 1435 strain include C16:0 and C18:0. In an effort to minimize C16:0 fatty acid levels, we investigated if optimizing PmKASII gene expression might result in further reductions in palmitate, thereby reducing total saturated fatty acids levels. A total of 14 putative strong, endogenous promoters were utilized to drive the expression of PmKASII gene (Table 53). These promoters were individually cloned upstream of the PmKASII gene as part of a cassette which simultaneously knocks out a single allele of FATA.

TABLE 53
Endogenous promoters identified through transcriptome analysis and
evaluated in this study: PmUAPA1 (Uric acid xanthine permease 1); PmHXT1 (Hexose co-
transporter); PmSAD2-2 (Stearoyl ACP desaturase 2-2); PmSOD (Superoxide dismutase);
PmATPB1 (ATP synthase subunit B); PmEF1-1 (Elongation factor allele 1); PmEF1-2
(Elongation factor allele 2); PmACP-P1(Acyl carrier protein plastidic-1); PmACP-P2 (Acyl
carrier protein plastidic-2); PmC1LYR1 (Homology to C1 LYR family domain); PmAMT1-1
(Ammonium transporter 1-1) PmAMT1-2 (Ammonium transporter 1-2); PmAMT3-1
(Ammonium transporter 3-1); PmAMT3-2 (Ammonium transporter 3-2)

| pSZ# | Construct |
|---|---|
| pSZ2533 | FATA1 3′::CrTUB2-ScSUC2-CvNR:PmUAPA1-CpSADtp-PmKASII- |
| | CvNR::FATA1 5′ |
| pSZ3869 | FATA1 3′::CrTUB2-ScSUC2-CvNR:PmHXT1-CpSADtp-PmKASII- |
| | CvNR::FATA1 5′ |
| pSZ3870 | FATA1 3′::CrTUB2-ScSUC2-CvNR:PmSAD2-2-CpSADtp-PmKASII- |
| | CvNR::FATA1 5′ |
| pSZ3935 | FATA1 3′::CrTUB2-ScSUC2-CvNR:PmSOD-CpSADtp-PmKASII-CvNR::FATA1 |
| | 5′ |
| pSZ3936 | FATA1 3′::CrTUB2-ScSUC2-CvNR:PmATPB1-CpSADtp-PmKASII- |
| | CvNR::FATA1 5′ |
| pSZ3937 | FATA1 3′::CrTUB2-ScSUC2-CvNR-PmEF1-1-CpSADtp-PmKASII- |
| | CvNR::FATA1 5′ |
| pSZ3938 | FATA1 3′::CrTUB2-ScSUC2-CvNR-PmEF1-2-CpSADtp-PmKASII- |
| | CvNR::FATA1 5′ |
| pSZ3939 | FATA1 3′::CrTUB2-ScSUC2-CvNR:PmACP-P1-CpSADtp-PmKASII- |
| | CvNR::FATA1 5′ |
| pSZ3940 | FATA1 3′::CrTUB2-ScSUC2-CvNR:PmACP-P2-CpSADtp-PmKASII- |
| | CvNR::FATA1 5′ |
| pSZ3941 | FATA1 3′::CrTUB2-ScSUC2-CvNR:PmC1LYR1-CpSADtp-PmKASII- |
| | CvNR::FATA1 5′ |
| pSZ3942 | FATA1 3′::CrTUB2-ScSUC2-CvNR:PmAMT1-1-CpSADtp-PmKASII- |
| | CvNR::FATA1 5′ |
| pSZ3943 | FATA1 3′::CrTUB2-ScSUC2-CvNR:PmAMT1-2-CpSADtp-PmKASII- |
| | CvNR::FATA1 5′ |
| pSZ3944 | FATA1 3′::CrTUB2-ScSUC2-CvNR:PmAMT3-1-CpSADtp-PmKASII- |
| | CvNR::FATA1 5′ |
| pSZ3945 | FATA1 3′::CrTUB2-ScSUC2-CvNR:PmAMT3-2-CpSADtp-PmKASII- |
| | CvNR::FATA1 5′ |

All the 14 constructs have same configuration except the different promoters that drive the expression of PmKASII gene. The sequences of these transforming DNAs are provided in the sequences below. In these constructs, the Saccharomyces cerevisiae invertase gene (SUC2) was utilized as the selectable marker, conferring on strains the ability to grow on sucrose. The resulting constructs were first transformed into high oleic base strain S5100, and a minimum of 20 transgenic lines arising from each transformation were assayed. As shown in Table 54, transgenic lines overexpressing the PmKASII gene that driven by promoters such as PmSAD2-2, PmACP-P1, PmACP-P2, PmUAPA1, and PmHXT1, show significant decreases in C16:0 fatty acid levels. We also observed a significant accumulation of C18:1 fatty acids.

We then transformed these top five constructs (PmSAD2-2, PmACP-P1, PmACP-P2, PmUAPA1, and PmHXT1) into high oleic strain S7505. Again, a minimum of 20 transgenic lines were assayed. Overall, the average C16:0 level achieved by transgenic lines generated in S7505 are lower than those generated in S5100, which is consistent with the levels observed in the parental strains. On the other hand, the promoter which resulted in the lowest C16:0 level, was different depending upon which high oleic base strain was tested. For example, PmACP-P2 appears to be the best promoter driving the expression of PmKASII in S5100, while in S7505, the PmSAD2-2 promoter performs the best (Table 54).

TABLE 54
Palmitate levels achieved in transgenic lines over expressing PmKASII
concomitant with down regulation of FATA1 in the high oleic base strains
S5100 and S7505. The lowest and average C16:0 levels are the result
of assessing a minimum of 20 transgenic lines from each transformation.

| | Parental | | Parental | |
|---|---|---|---|---|
| | strain S5100 | | strain S7505 | |
| | Lowest | Average | lowest | Average |
| Constructs | C16:0 | C16:0 | C16:0 | C16:0 |
| PmUAPA1::PmKASII, Δfata1 | 3.88 | 8.78 | 4.74 | 7.99 |
| PmHXT1::PmKASII, Δfata1 | 4.37 | 9.47 | 5.99 | 8.09 |
| PmSAD2-2::PmKASII, Δfata1 | 3.82 | 8.36 | 2.38 | 5.88 |
| PmSOD::PmKASII, Δfata1 | 7.71 | 9.83 | — | — |
| PmATPB1::PmKASII, Δfata1 | 10.11 | 13.97 | — | — |
| PmEF1-1::PmKASII, Δfata1 | 8.29 | 8.91 | — | — |
| PmEF1-2::PmKASII, Δfata1 | 8.47 | 10.15 | — | — |
| PmACP-P1::PmKASII, Δfata1 | 3.03 | 7.93 | 3.09 | 6.94 |
| PmACP-P2::PmKASII, Δfata1 | 3.01 | 7.81 | 3.55 | 6.63 |
| PmC1LYR1::PmKASII, Δfata1 | 10.31 | 11.45 | — | — |
| PmAMT1-1::PmKASII, Δfata1 | 6.51 | 9.62 | — | — |
| PmAMT1-2::PmKASII, Δfata1 | 5.21 | 8.56 | — | — |
| PmAMT3-1::PmKASII, Δfata1 | 6.37 | 10.72 | — | — |
| PmAMT3-2::PmKASII, Δfata1 | 9.69 | 10.83 | — | — |

Given the initial results seen through the inactivation of FATA1 and overexpression of PmKASII when driven by the PmSAD2-2 promoter in strain S7505, we moved several of these transgenic lines into genetic stability assays and assessment of the integration events by Southern blot analysis. Strain S7740 is a resulting stable line showing the correct integration of the DNA into the FATA1 locus. The fatty acid profile of S7740 when evaluated in lab scale fermenter is shown in Table 55. As expected, the C16:0 levels in strain S7740 are 2.3% lower than that observed in previous high oleic leading strain S5587 run under the same conditions (Table 55). S5587 is a strain in which pSZ2533 was expressed in S5100.

TABLE 55
Comparison of fatty acid profiles between strains S5587 and S7740 in
high cell-density fermentation experiment. Strain S7740 produces
2.3% less C16:0 than S5587, while the oleate levels are comparable
between the two strains.

| | Fatty Acid area % | | | | | |
|---|---|---|---|---|---|---|
| Strains | C16:0 | C18:0 | C18:1 | C18:2 | C20:1 | Total saturates |
| S5587 | 3.7 | 3.5 | 85.6 | 5.6 | 0.7 | 7.9 |
| S7740 | 1.4 | 4.9 | 85.2 | 5.1 | 2.1 | 7.3 |

S7740 is one of the transformants generated from pSZ3870 (FATA13′::CrTUB2: ScSUC2:CvNR::PmSAD2-2-CpSADtp:PmKASII-CvNR::FATA1 5′) transforming S7505. The sequence of the pSZ3870 transforming DNA is provided in SEQ ID NO: 66. Relevant restriction sites in the construct are indicated in lowercase, bold and underlining and are 5′-3′ BspQ 1, Kpn I, Asc I, Mfe I, EcoRV, SpeI, AscI, ClaI, Sac I, BspQ I, respectively. BspQI sites delimit the 5′ and 3′ ends of the transforming DNA. Bold, lowercase sequences represent FATA1 3′ genomic DNA that permit targeted integration at FATA1 locus via homologous recombination. Proceeding in the 5′ to 3′ direction, the C. reinhardtii β-tubulin promoter driving the expression of the yeast sucrose invertase gene is indicated by boxed text. The initiator ATG and terminator TGA for invertase are indicated by uppercase, bold italics while the coding region is indicated in lowercase italics. The Chlorella vulgaris nitrate reductase 3′ UTR is indicated by lowercase underlined text followed by the P. moriformis SAD2-2 promoter, indicated by boxed italics text. The Initiator ATG and terminator TGA codons of the PmKASII are indicated by uppercase, bold italics, while the remainder of the coding region is indicated by bold italics. The Chlorella protothecoides S106 stearoyl-ACP desaturase transit peptide is located between initiator ATG and the Asc I site. The C. vulgaris nitrate reductase 3′ UTR is again indicated by lowercase underlined text followed by the FATA1 5′ genomic region indicated by bold, lowercase text.

As we described earlier, we utilized 13 additional promoters for driving the expression of PmKASII. All 14 constructs have same configuration and relevant restriction sites.

Nucleotide sequence of transforming DNA contained in pSZ3870:
(SEQ ID NO: 66)
gctcttcacccaactcagataataccaatacccctccttctcctcctcatccattcagtacccccccccttctcttcccaaagcagcaagcgcgtg
gcttacagaagaacaatcggcttccgccaaagtcgccgagcactgcccgacggcggcgcgcccagcagcccgcttggccacacaggcaacga
atacattcaatagggggcctcgcagaatggaaggagcggtaaagggtacaggagcactgcgcacaaggggcctgtgcaggagtgactgact
gggcgggcagacggcgcaccgcgggcgcaggcaagcagggaagattgaagcggcagggaggaggatgctgattgaggggggcatcgcagt
ctctcttggacccgggataaggaagcaaatattcggccggttgggttgtgtgtgtgcacgttttcttcttcagagtcgtgggtgtgcttccaggga

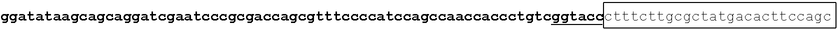

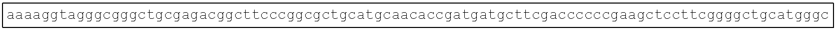

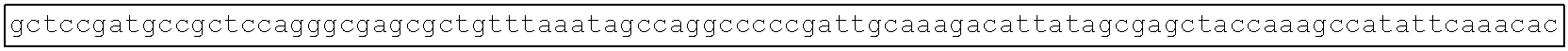

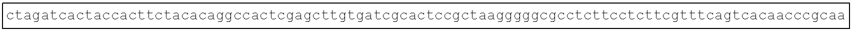

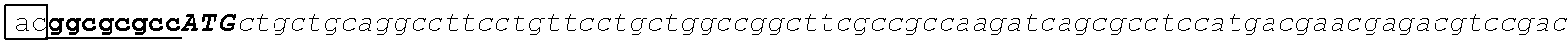

cgccccctggtgcacttcacccccaacaagggctggatgaacgaccccaacggcctgtggtacgacgagaaggacgccaagtggcacct
gtacttccagtacaacccgaacgacaccgtctgggggacgcccttgttctggggccacgccacgtccgacgacctgaccaactgggagga
ccagcccatcgccatcgccccgaagcgcaacgactccggcgccttctccggctccatggtggtggactacaacaacacctccggcttcttca
acgacaccatcgacccgcgccagcgctgcgtggccatctggacctacaacaccccggagtccgaggagcagtacatctcctacagcctgg
acggcggctacaccttcaccgagtaccagaagaaccccgtgctggccgccaactccacccagttccgcgacccgaaggtcttctggtacga
gccctcccagaagtggatcatgaccgcggccaagtcccaggactacaagatcgagatctactcctccgacgacctgaagtcctggaagct
ggagtccgcgttcgccaacgagggcttcctcggctaccagtacgagtgccccggcctgatcgaggtccccaccgagcaggaccccagcaa
gtcctactgggtgatgttcatctccatcaaccccggcgccccggccggcggctccttcaaccagtacttcgtcggcagcttcaacggcaccca
cttcgaggccttcgacaaccagtcccgcgtggtggacttcggcaaggactactacgccctgcagaccttcttcaacaccgacccgacctacg
ggagcgccctgggcatcgcgtgggcctccaactgggagtactccgccttcgtgcccaccaacccctggcgctcctccatgtccctcgtgcgca
agttctccctcaacaccgagtaccaggccaacccggagacggagctgatcaacctgaaggccgagccgatcctgaacatcagcaacgcc
ggcccctggagccggttcgccaccaacaccacgttgacgaaggccaacagctacaacgtcgacctgtccaacagcaccggcaccctgga
gttcgagctggtgtacgccgtcaacaccacccagacgatctccaagtccgtgttcgcggacctctccctctggttcaagggcctggaggacc
ccgaggagtacctccgcatgggcttcgaggtgtccgcgtcctccttcttcctggaccgcgggaacagcaaggtgaagttcgtgaaggaga
acccctacttcaccaaccgcatgagcgtgaacaaccagcccttcaagagcgagaacgacctgtcctactacaaggtgtacggcttgctgga
ccagaacatcctggagctgtacttcaacgacggcgacgtcgtgtccaccaacacctacttcatgaccaccgggaacgccctgggctccgtg

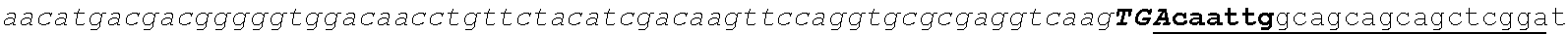

agtatcgacacactctggacgctggtcgtgtgatggactgttgccgccacacttgctgccttgacctgtgaatatccctgccgcttttatcaaacag
cctcagtgtgtttgatcttgtgtgtacgcgcttttgcgagttgctagctgcttgtgctatttgcgaataccacccccagcatccccttccctcgtttcat
atcgcttgcatcccaaccgcaacttatctacgctgtcctgctatccctcagcgctgctcctgctcctgctcactgcccctcgcacagccttggtttgg
gctccgcctgtattctcctggtactgcaacctgtaaaccagcactgcaatgctgatgcacgggaagtagtgggatgggaacacaaatggaggat
cccgcgtctcgaacagagcgcgcagaggaacgctgaaggtctcgcctctgtcgcacctcagcgcggcatacaccacaataaccacctgacgaa
tgcgcttggttcttcgtccattagcgaagcgtccggttcacacacgtgccacgttggcgaggtggcaggtgacaatgatcggtggagctgatggtc

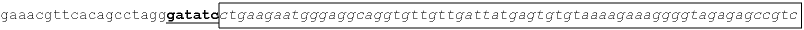

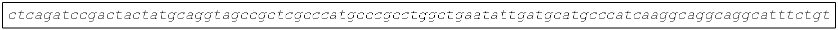

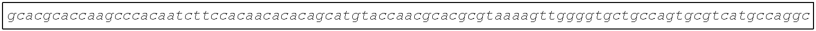

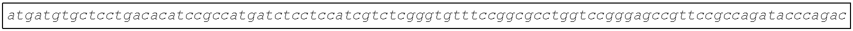

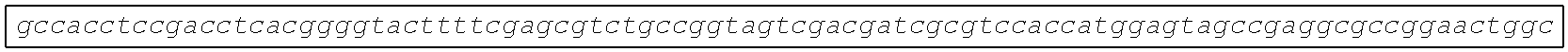

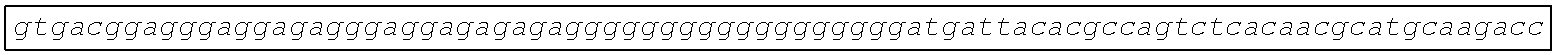

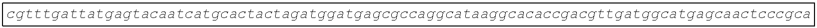

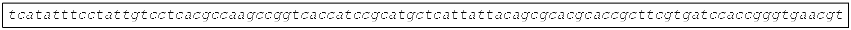

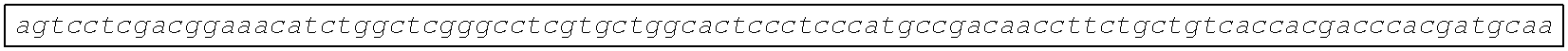

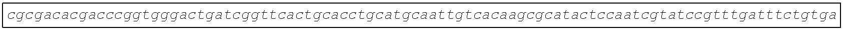

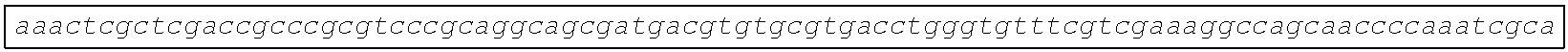

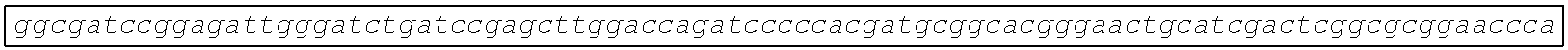

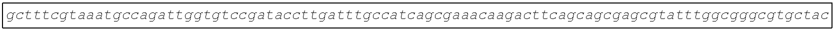

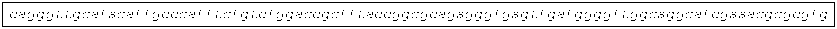

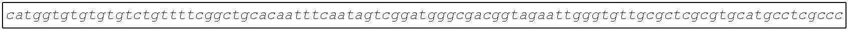

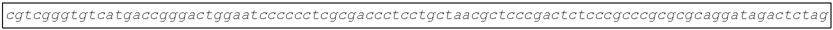

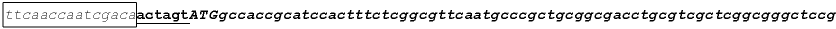

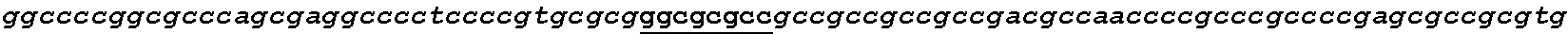

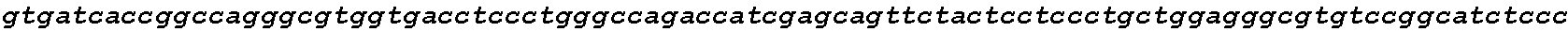

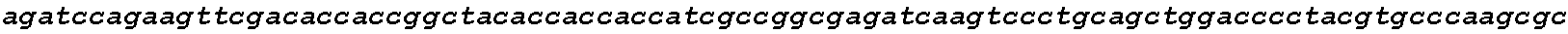

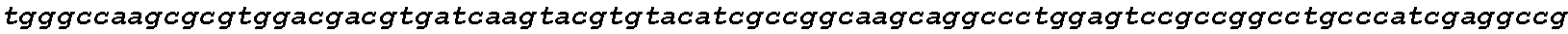

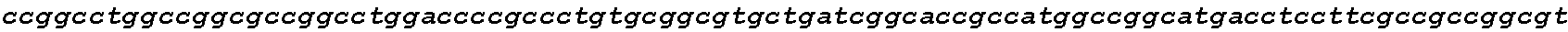

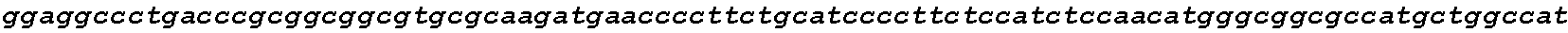

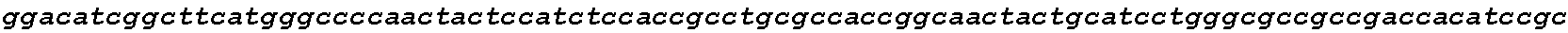

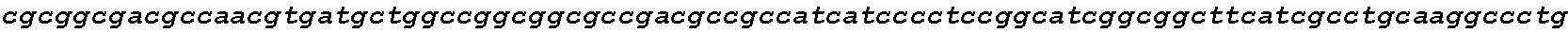

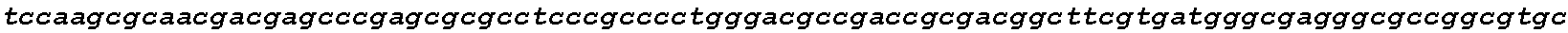

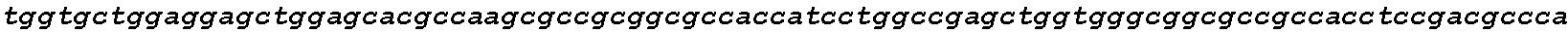

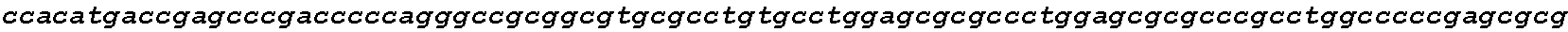

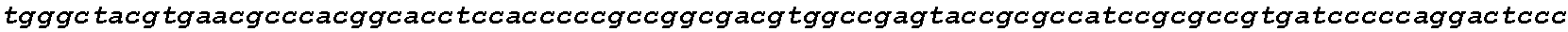

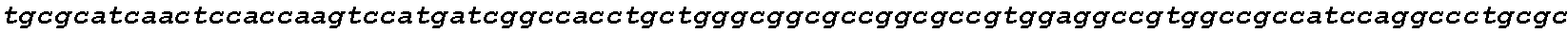

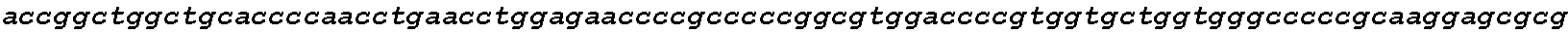

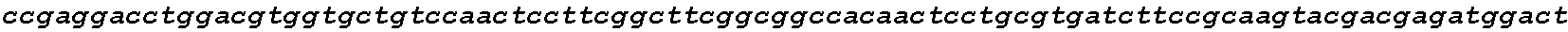

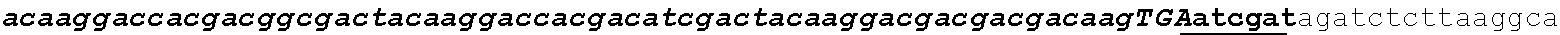

gcagcagctcggatagtatcgacacactctggacgctggtcgtgtgatggactgttgccgccacacttgctgccttgacctgtgaatatccctgcc
gcttttatcaaacagcctcagtgtgtttgatcttgtgtgtacgcgcttttgcgagttgctagctgcttgtgctatttgcgaataccacccccagcatcc
ccttccctcgtttcatatcgcttgcatcccaaccgcaacttatctacgctgtcctgctatccctcagcgctgctcctgctcctgctcactgcccctcgc
acagccttggtttgggctccgcctgtattctcctggtactgcaacctgtaaaccagcactgcaatgctgatgcacgggaagtagtgggatggaa
cacaaatggaaagcttaattaagagctcttgttttccagaaggagttgctccttgagcctttcattctcagcctcgataacctccaaagccgctct
aattgtggagggggttcgaaccgaatgctgcgtgaacgggaaggaggaggagaaagagtgagcagggagggattcagaaatgagaaatg
agaggtgaaggaacgcatccctatgcccttgcaatggacagtgtttctggccaccgccaccaagacttcgtgtcctctgatcatcatgcgattga
ttacgttgaatgcgacggccggtcagccccggacctccacgcaccggtgctcctccaggaagatgcgcttgtcctccgccatcttgcagggctca
agctgctcccaaaactcttgggcgggttccggacggacggctaccgcgggtgcggccctgaccgccactgttcggaagcagcggcgctgcatg
ggcagcggccgctgcggtgcgccacggaccgcatgatccaccggaaaagcgcacgcgctggagcgcgcagaggaccacagagaagcggaa
gagacgccagtactggcaagcaggctggtcggtgccatggcgcgctactaccctcgctatgactcgggtcctcggccggctggcggtgctgaca
attcgtttagtggagcagcgactccattcagctaccagtcgaactcagtggcacagtgactccgctcttc
Nucleotide sequence of PmUAPA1 promoter contained in pSZ2533:
(SEQ ID NO: 67)

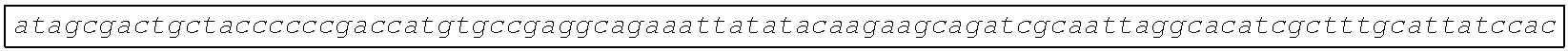

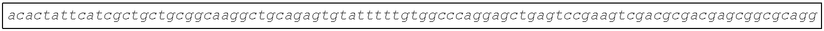

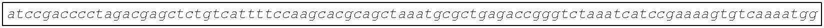

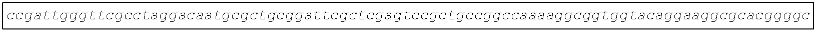

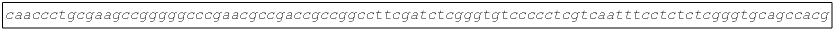

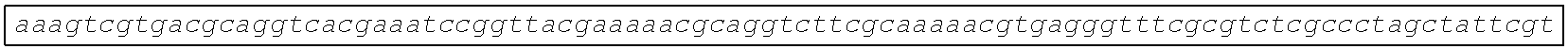

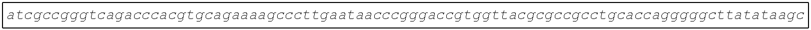

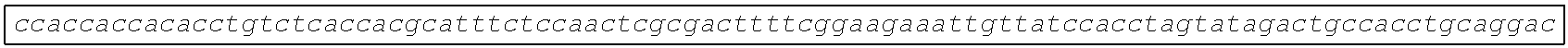

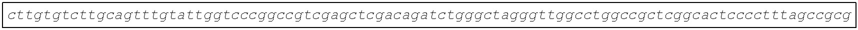

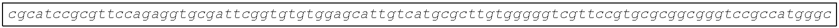

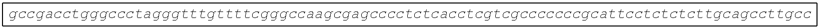

Nucleotide sequence of PmHXT1 promoter contained in pSZ3869:
(SEQ ID NO: 68)

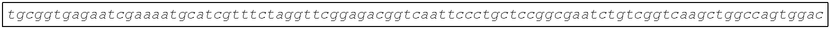

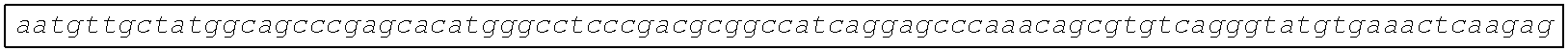

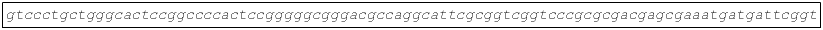

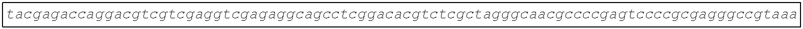

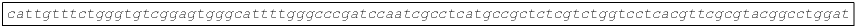

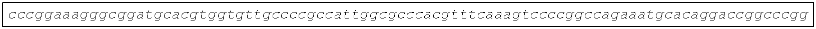

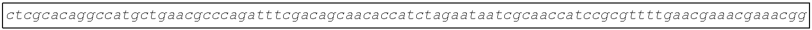

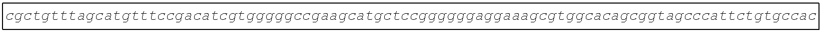

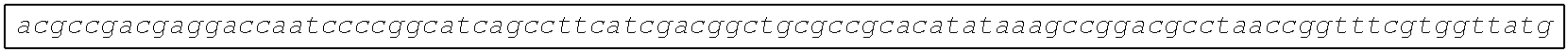

Nucleotide sequence of PmSOD promoter contained in pSZ3935:
(SEQ ID NO: 69)

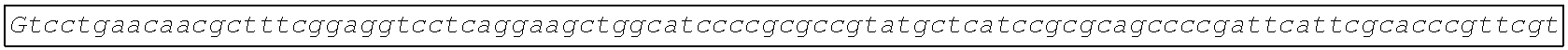

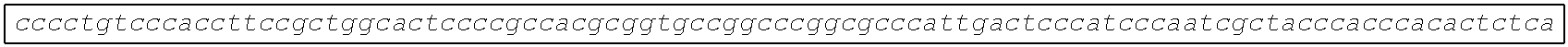

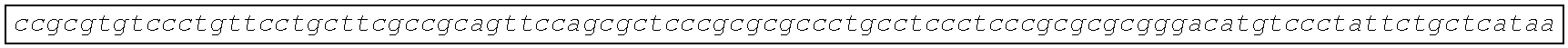

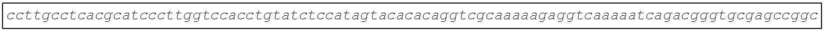

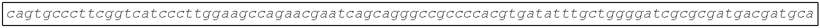

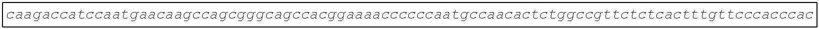

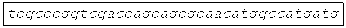

Nucleotide sequence of PmATPB1 promoter contained in pSZ3936:
(SEQ ID NO: 70)

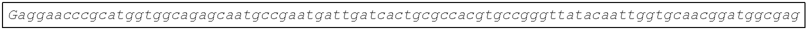

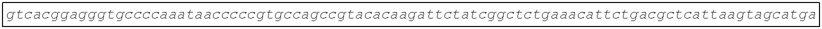

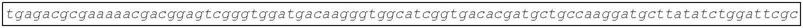

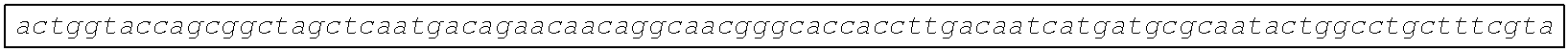

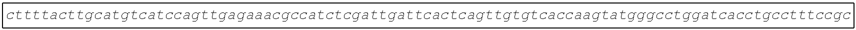

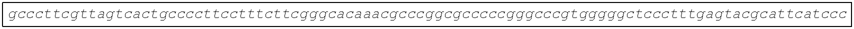

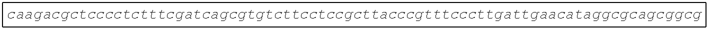

Nucleotide sequence of PmEf1-1 promoter contained in pSZ3937:
(SEQ ID NO: 71)

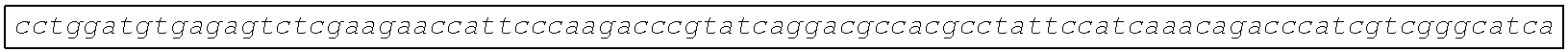

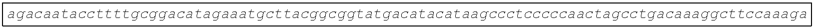

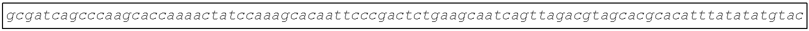

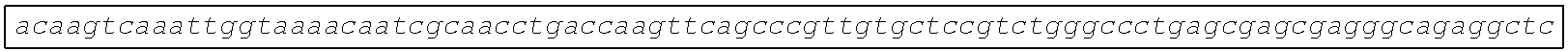

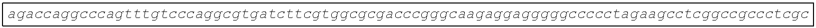

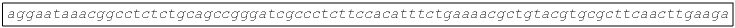

Nucleotide sequence of PmEf1-2 promoter contained in pSZ3938:
(SEQ ID NO: 72)

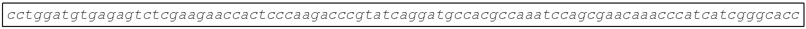

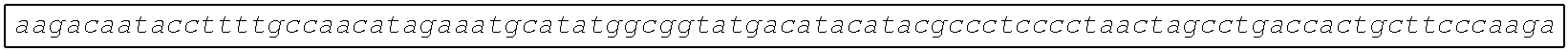

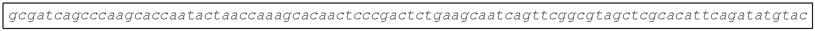

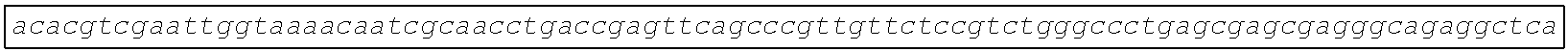

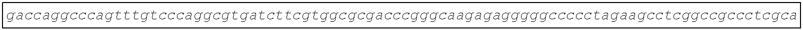

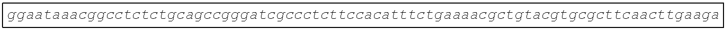

Nucleotide sequence of PmACP1 promoter contained in pSZ3939:
(SEQ ID NO: 73)

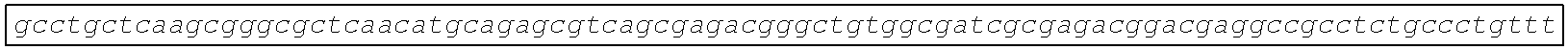

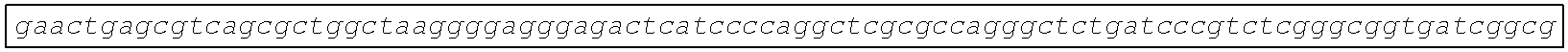

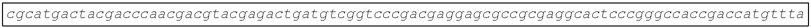

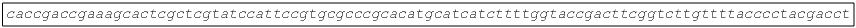

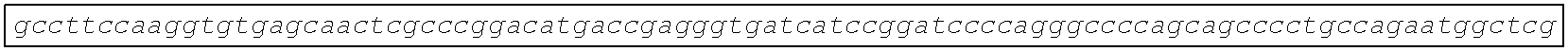

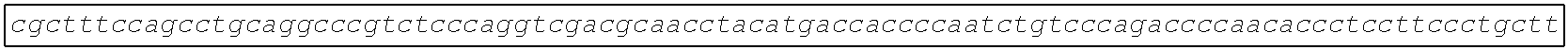

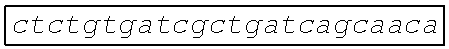

Nucleotide sequence of PmACP2 promoter contained in pSZ3940:
(SEQ ID NO: 74)

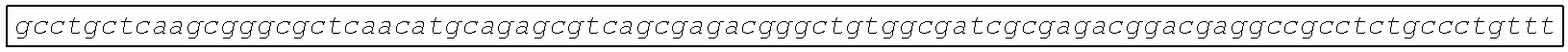

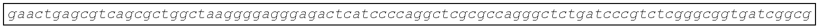

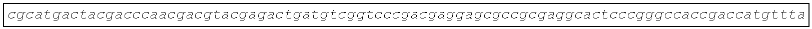

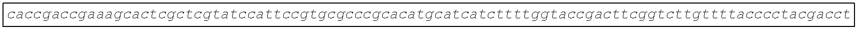

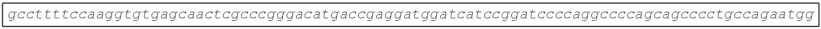

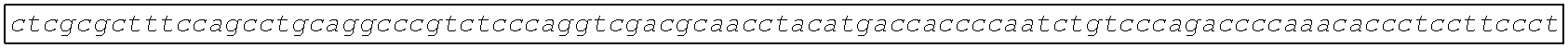

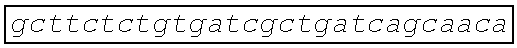

Nucleotide sequence of PmC1LYR1 promoter contained in pSZ3941:
(SEQ ID NO: 75)

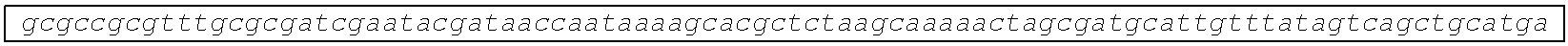

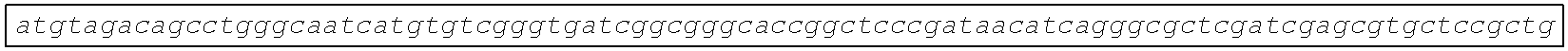

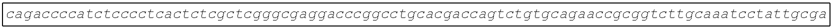

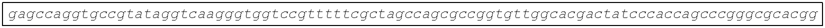

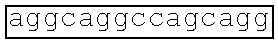

Nucleotide sequence of PmAMT1-1 promoter contained in pSZ3942:
(SEQ ID NO: 76)

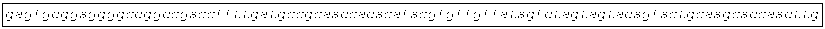

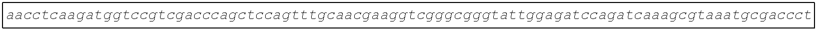

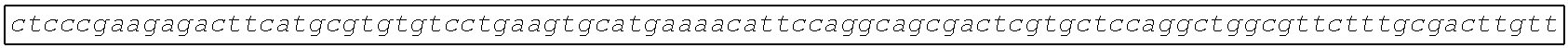

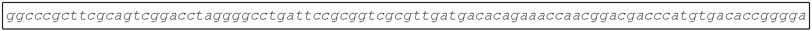

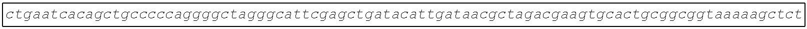

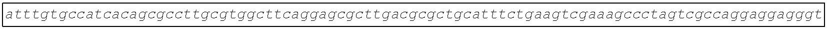

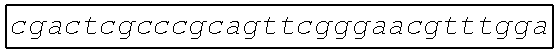

Nucleotide sequence of PmAMT1-2 promoter contained in pSZ3943:
(SEQ ID NO: 77)

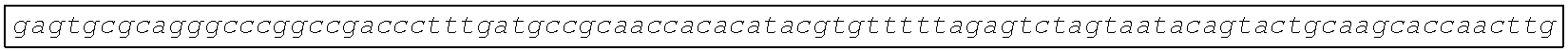

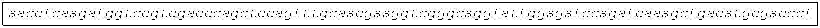

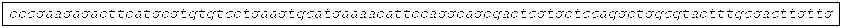

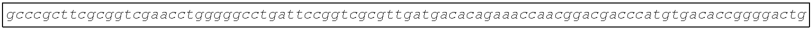

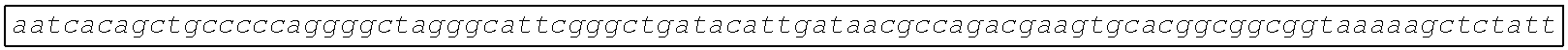

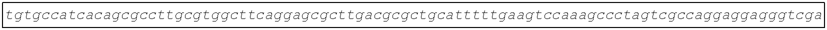

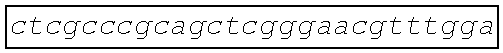

Nucleotide sequence of PmAMT3-1 promoter contained in pSZ3944:
(SEQ ID NO: 78)

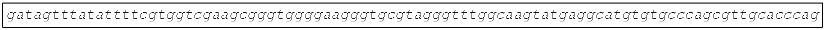

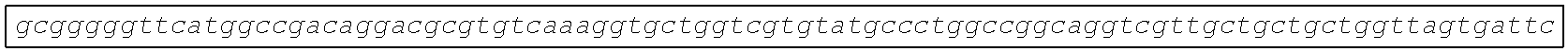

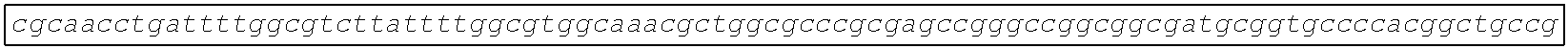

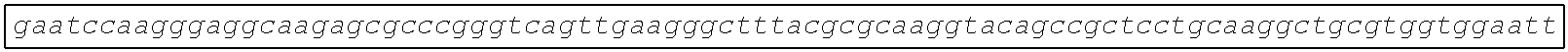

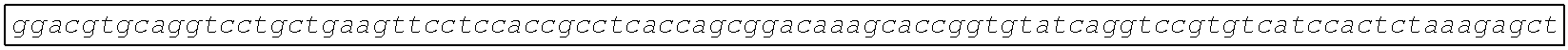

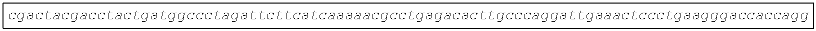

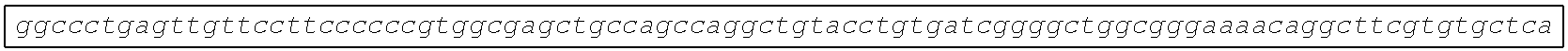

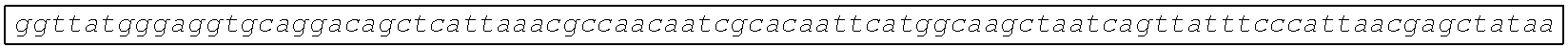

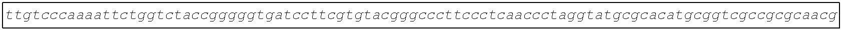

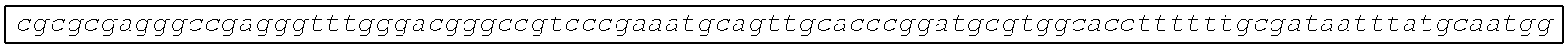

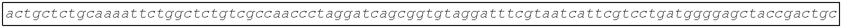

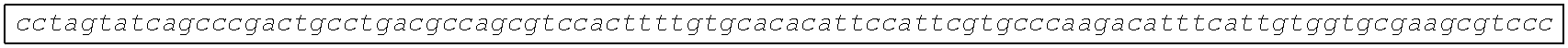

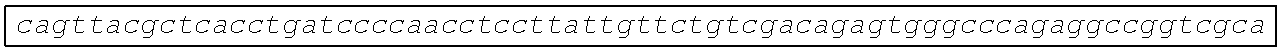

Nucleotide sequence of PmAMT3-2 promoter contained in pSZ3945:
(SEQ ID NO: 79)

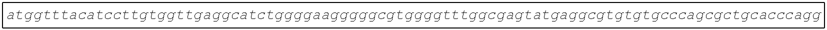

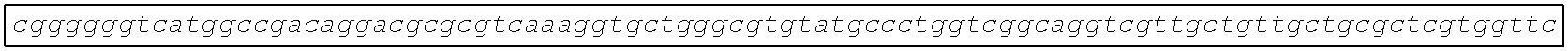

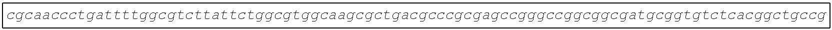

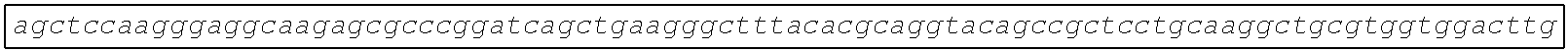

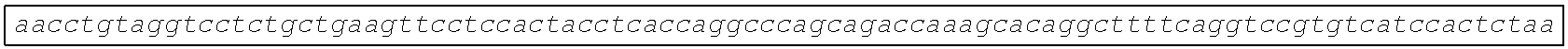

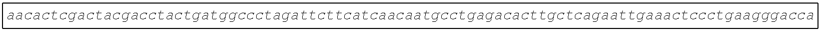

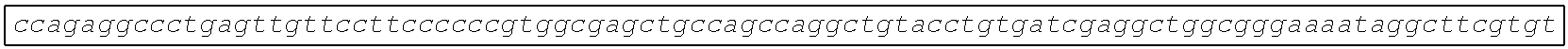

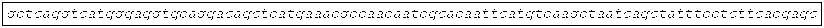

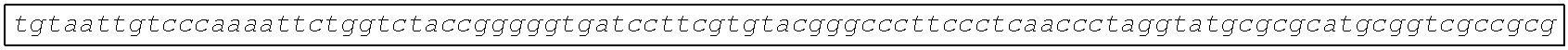

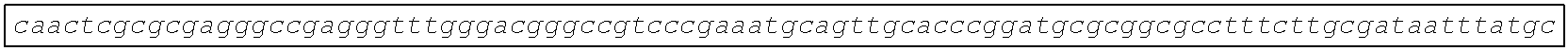

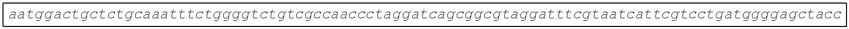

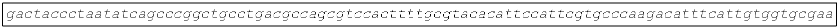

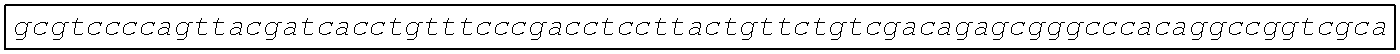

Expression of PmSAD2-1 in S7740 Resulted in Zero SAT FAT Strain S8188

The PmSAD2-1 gene was then introduced into S7740 to reduce the stearic level. Strain S8188 is one of the stable lines generated from the transformation of pSZ4768 DNA (FAD2 5′::PmHXT1V2-ScarMEL1-PmPGK:PmSAD2-2p-CpSADtp-PmKASII-CvNR:PmACP1-PmSAD2-1-CvNR::FAD2 3′) into S7740. In this construct, the Saccharomyces carlbergensis MEL1 gene was used as the selectable marker to introduce the PmSAD2-1, and an additional copy of PmKASII into the FAD2-1 locus of P. moriformis strain S7740 by homologous recombination using previously described transformation methods (biolistics).

The sequence of the pSZ4768 (D3870) transforming DNA is provided in SEQ ID NO: 85. Relevant restriction sites in pSZ4768 are indicated in lowercase, bold and underlining and are 5′-3′ BspQ 1, Kpn I, SnaBI, BamHI, AvrII, SpeI, AscI, ClaI, EcoRI, SpeI, AscI, ClaI, PacI, SacI BspQ I, respectively. BspQI sites delimit the 5′ and 3′ ends of the transforming DNA. Bold, lowercase sequences represent FAD2-1 5′ genomic DNA that permits targeted integration at FAD2-1 locus via homologous recombination. Proceeding in the 5′ to 3′ direction, the P. moriformis HXT1 promoter driving the expression of the S. carlbergensis MEL1 gene is indicated by boxed text. The initiator ATG and terminator TGA for ScarMEL1 are indicated by uppercase, bold italics while the coding region is indicated in lowercase italics. The P. moriformis PGK 3′UTR is indicated by lowercase underlined text followed by the PmSAD2-2 promoter indicated by boxed italics text. The Initiator ATG and terminator TGA codons of the PmKASII are indicated by uppercase, bold italics, while the remainder of the coding region is indicated by bold italics. The Chlorella protothecoides S106 stearoyl-ACP desaturase transit peptide is located between initiator ATG and the Asc I site. The Chlorella vulgaris nitrate reductase 3′ UTR is indicated by lowercase underlined text followed by the PmACP1 promoter driving the expression of PmSAD2-1 gene. The PmACP1 promoter is indicated by boxed italics text. The Initiator

ATG and terminator TGA codons of the PmSAD2-1 are indicated by uppercase, bold italics, while the remainder of the coding region is indicated by bold italics. The C. protothecoides S106 stearoyl-ACP desaturase transit peptide is located between initiator ATG and the Asc I site. The C. vulgaris nitrate reductase 3′ UTR is again indicated by lowercase underlined text followed by the FAD2-1 3′ genomic region indicated by bold, lowercase text.

Nucleotide sequence of transforming DNA contained in pSZ4768 (D3870):
(SEQ ID NO: 80)
gctcttcgcgaaggtcattttccagaacaacgaccatggcttgtcttagcgatcgctcgaatgactgctagtgagtcgtacgctcgacccagtcg
ctcgcaggagaacgcggcaactgccgagcttcggcttgccagtcgtgactcgtatgtgatcaggaatcattggcattggtagcattataattcg
gcttccgcgctgtttatgggcatggcaatgtctcatgcagtcgaccttagtcaaccaattctgggtggccagctccgggcgaccgggctccgtgt
cgccgggcaccacctcctgccatgagtaacagggccgccctctcctcccgacgttggcccactgaataccgtgtcttggggccctacatgatggg
ctgcctagtcgggcgggacgcgcaactgcccgcgcaatctgggacgtggtctgaatcctccaggcgggtttccccgagaaagaaagggtgccg
atttcaaagcagagccatgtgccgggccctgtggcctgtgttggcgcctatgtagtcaccccccctcacccaattgtcgccagtttgcgcaatcc

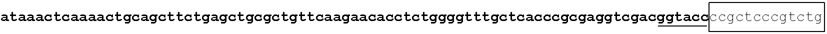

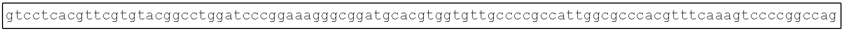

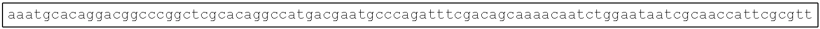

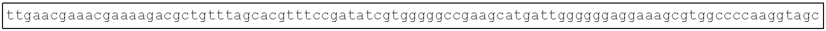

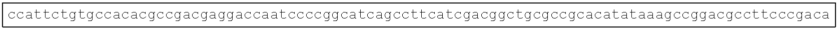

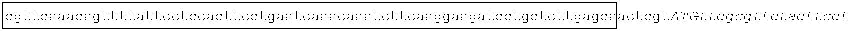

gacggcctgcatctccctgaagggcgtgttcggcgtctccccctcctacaacggcctgggcctgacgccccagatgggctgggacaactgg
aacacgttcgcctgcgacgtctccgagcagctgctgctggacacggccgaccgcatctccgacctgggcctgaaggacatgggctacaag
tacatcatcctggacgactgctggtcctccggccgcgactccgacggcttcctggtcgccgacgagcagaagttccccaacggcatgggcc
acgtcgccgaccacctgcacaacaactccttcctgttcggcatgtactcctccgcgggcgagtacacgtgcgccggctaccccggctccctgg
gccgcgaggaggaggacgcccagttcttcgcgaacaaccgcgtggactacctgaagtacgacaactgctacaacaagggccagttcggc
acgcccgagatctcctaccaccgctacaaggccatgtccgacgccctgaacaagacgggccgccccatcttctactccctgtgcaactgggg
ccaggacctgaccttctactggggctccggcatcgcgaactcctggcgcatgtccggcgacgtcacggcggagttcacgcgccccgactccc
gctgcccctgcgacggcgacgagtacgactgcaagtacgccggcttccactgctccatcatgaacatcctgaacaaggccgcccccatggg
ccagaacgcgggcgtcggcggctggaacgacctggacaacctggaggtcggcgtcggcaacctgacggacgacgaggagaaggcgca
cttctccatgtgggccatggtgaagtcccccctgatcatcggcgcgaacgtgaacaacctgaaggcctcctcctactccatctactcccaggc
gtccgtcatcgccatcaaccaggactccaacggcatccccgccacgcgcgtctggcgctactacgtgtccgacacggacgagtacggccag
ggcgagatccagatgtggtccggccccctggacaacggcgaccaggtcgtggcgctgctgaacggcggctccgtgtcccgccccatgaac
acgaccctggaggagatcttcttcgactccaacctgggctccaagaagctgacctccacctgggacatctacgacctgtgggcgaaccgcg
tcgacaactccacggcgtccgccatcctgggccgcaacaagaccgccaccggcatcctgtacaacgccaccgagcagtcctacaaggacg
gcctgtccaagaacgacacccgcctgttcggccagaagatcggctccctgtcccccaacgcgatcctgaacacgaccgtccccgcccacgg

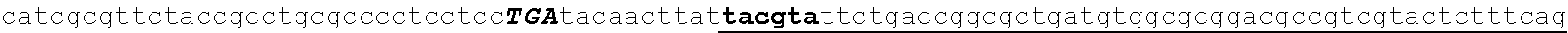

actttactcttgaggaattgaacctttctcgcttgctggcatgtaaacattggcgcaattaattgtgtgatgaagaaagggtggcacaagatggat
cgcgaatgtacgagatcgacaacgatggtgattgttatgaggggccaaacctggctcaatcttgtcgcatgtccggcgcaatgtgatccagcggc
gtgactctcgcaacctggtagtgtgtgcgcaccgggtcgctttgattaaaactgatcgcattgccatcccgtcaactcacaagcctactctagctcc
cattgcgcactcgggcgcccggctcgatcaatgttctgagcggagggcgaagcgtcaggaaatcgtctcggcagctggaagcgcatggaatgcg
gagcggagatcgaatcaggatcccgcgtctcgaacagagcgcgcagaggaacgctgaaggtctcgcctctgtcgcacctcagcgcggcataca
ccacaataaccacctgacgaatgcgcttggttcttcgtccattagcgaagcgtccggttcacacacgtgccacgttggcgaggtggcaggtgaca

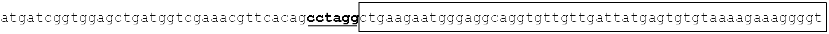

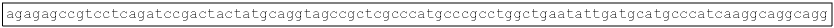

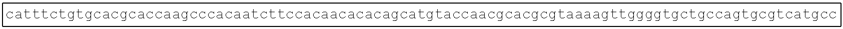

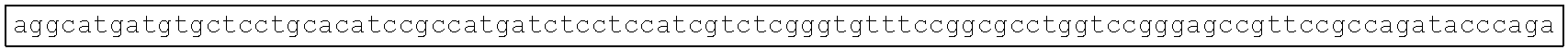

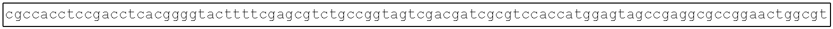

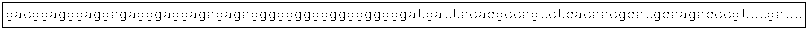

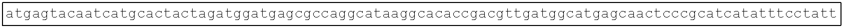

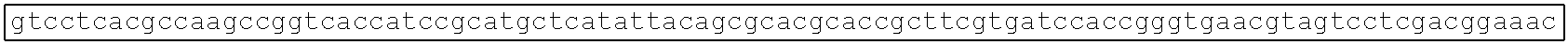

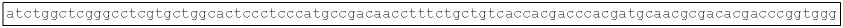

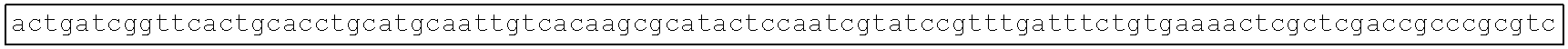

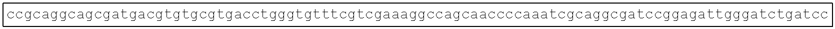

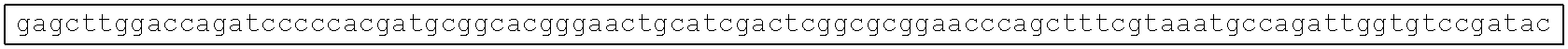

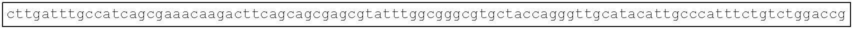

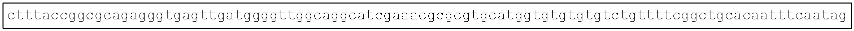

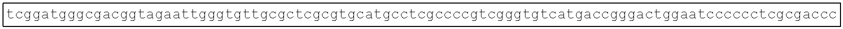

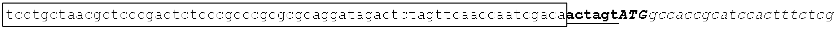

gcgttcaatgcccgctgcggcgacctgcgtcgctcggcgggctccgggccccggcgcccagcgaggcccctccccgtgcgcgggcgcgccg
ccgccgccgccgacgccaaccccgcccgccccgagcgccgcgtggtgatcaccggccagggcgtggtgacctccctgggccagaccatcg
agcagttctactcctccctgctggagggcgtgtccggcatctcccagatccagaagttcgacaccaccggctacaccaccaccatcgccggc
gagatcaagtccctgcagctggacccctacgtgcccaagcgctgggccaagcgcgtggacgacgtgatcaagtacgtgtacatcgccggc
aagcaggccctggagtccgccggcctgcccatcgaggccgccggcctggccggcgccggcctggaccccgccctgtgcggcgtgctgatc
ggcaccgccatggccggcatgacctccttcgccgccggcgtggaggccctgacccgcggcggcgtgcgcaagatgaaccccttctgcatcc
ccttctccatctccaacatgggcggcgccatgctggccatggacatcggcttcatgggccccaactactccatctccaccgcctgcgccaccg
gcaactactgcatcctgggcgccgccgaccacatccgccgcggcgacgccaacgtgatgctggccggcggcgccgacgccgccatcatcc
cctccggcatcggcggcttcatcgcctgcaaggccctgtccaagcgcaacgacgagcccgagcgcgcctcccgcccctgggacgccgaccg
cgacggcttcgtgatgggcgagggcgccggcgtgctggtgctggaggagctggagcacgccaagcgccgcggcgccaccatcctggccg
agctggtgggcggcgccgccacctccgacgcccaccacatgaccgagcccgacccccagggccgcggcgtgcgcctgtgcctggagcgcg
ccctggagcgcgcccgcctggcccccgagcgcgtgggctacgtgaacgcccacggcacctccacccccgccggcgacgtggccgagtacc
gcgccatccgcgccgtgatcccccaggactccctgcgcatcaactccaccaagtccatgatcggccacctgctgggcggcgccggcgccgt
ggaggccgtggccgccatccaggccctgcgcaccggctggctgcaccccaacctgaacctggagaaccccgcccccggcgtggaccccgt
ggtgctggtgggcccccgcaaggagcgcgccgaggacctggacgtggtgctgtccaactccttcggcttcggcggccacaactcctgcgtg
atcttccgcaagtacgacgagatggactacaaggaccacgacggcgactacaaggaccacgacatcgactacaaggacgacgacgac
aagTGAatcgatagatctcttaaggcagcagcagctcggatagtatcgacacactctggacgctggtcgtgtgatggactgttgccgccacact
tgctgccttgacctgtgaatatccctgccgcttttatcaaacagcctcagtgtgtttgatcttgtgtgtacgcgcttttgcgagttgctagctgcttgtg
ctatttgcgaataccacccccagcatccccttccctcgtttcatatcgcttgctcccaaccgcaacttatctacgctgtcctgctatccctcagcgct
gctcctgctcctgctcactgcccctcgcacagccttggtttgggctccgcctgtattctcctggtactgcaacctgtaaaccagcactgcaatgctga

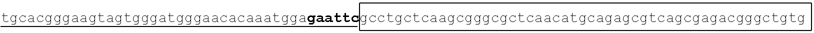

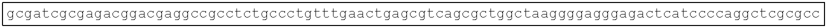

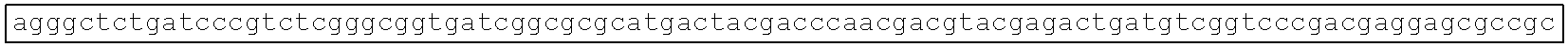

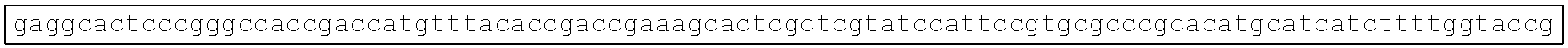

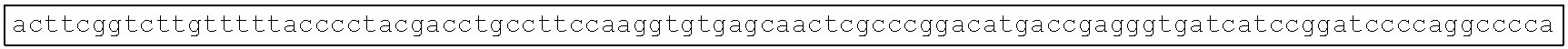

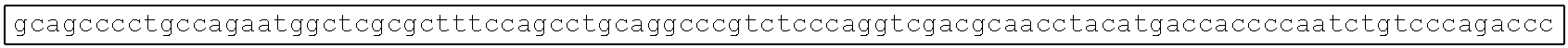

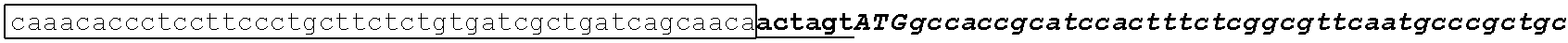

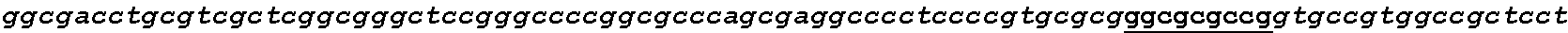

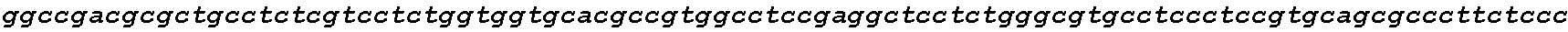

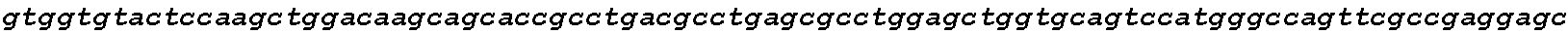

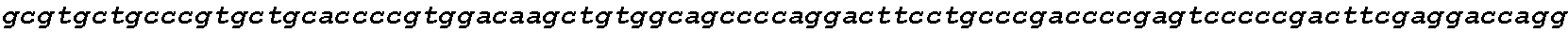

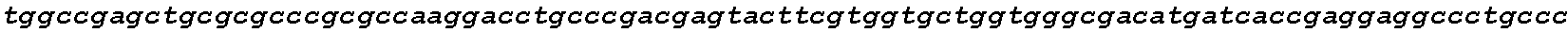

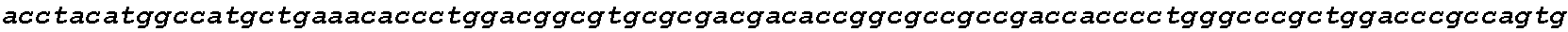

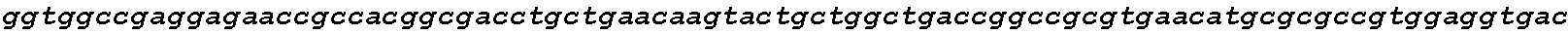

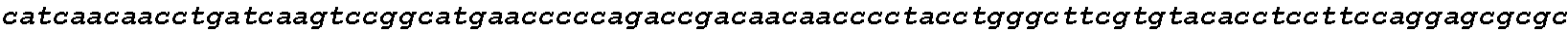

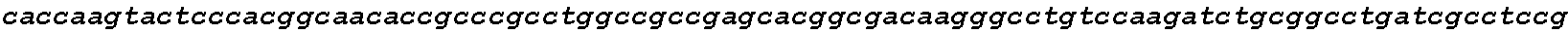

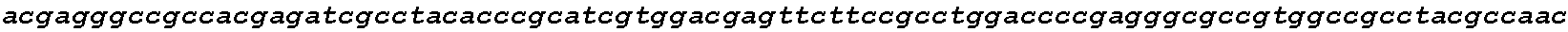

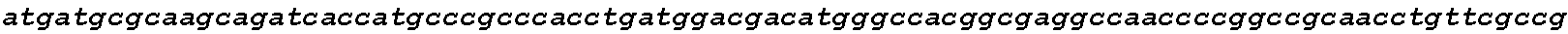

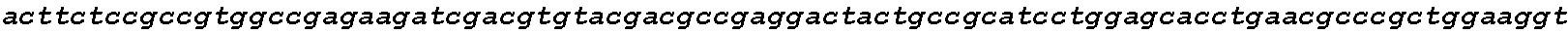

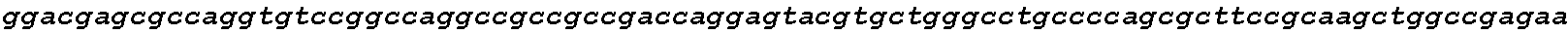

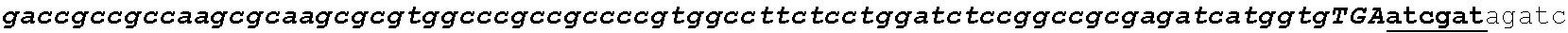

tcttaaggcagcagcagctcggatagtatcgacacactctggacgctggtcgtgtgatggactgttgccgccacacttgctgccttgacctgtgaa
tatccctgccgcttttatcaaacagcctcagtgtgtttgatcttgtgtgtacgcgcttttgcgagttgctagctgcttgtgctatttgcgaataccacc
cccagcatccccttccctcgtttcatatcgcttgcatcccaaccgcaacttatctacgctgtcctgctatccctcagcgctgctcctgctcctgctcac
tgcccctcgcacagccttggtttgggctccgcctgtattctcctggtactgcaacctgtaaaccagcactgcaatgctgatgcacgggaagtagtg
ggatgggaacacaaatggaaagcttaattaagagctcctcactcagcgcgcctgcgcggggatgcggaacgccgccgccgccttgtcttttgca
cgcgcgactccgtcgcttcgcgggtggcacccccattgaaaaaaacctcaattctgtttgtggaagacacggtgtacccccaaccacccacctg
cacctctattattggtattattgacgcgggagcgggcgttgtactctacaacgtagcgtctctggttttcagctggctcccaccattgtaaattctt
gctaaaatagtgcgtggttatgtgagaggtatggtgtaacagggcgtcagtcatgttggttttcgtgctgatctcgggcacaaggcgtcgtcga
cgtgacgtgcccgtgatgagagcaataccgcgctcaaagccgacgcatggcctttactccgcactccaaacgactgtcgctcgtatttttcggat
atctattttttaagagcgagcacagcgccgggcatgggcctgaaaggcctcgcggccgtgctcgtggtgggggccgcgagcgcgtggggcatc
gcggcagtgcaccaggcgcagacggaggaacgcatggtgagtgcgcatcacaagatgcatgtcttgttgtctgtactataatgctagagcatc
accaggggcttagtcatcgcacctgctttggtcattacagaaattgcacaagggcgtcctccgggatgaggagatgtaccagctcaagctgga
gcggcttcgagccaagcaggagcgcggcgcatgacgacctacccacatgcgaagagc

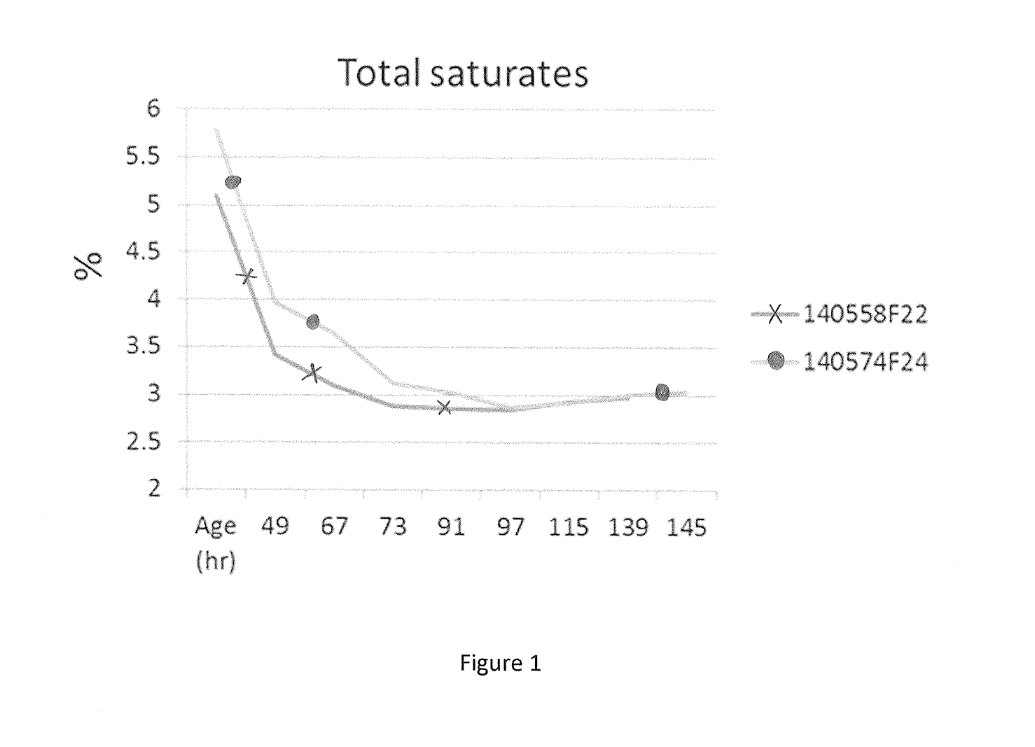

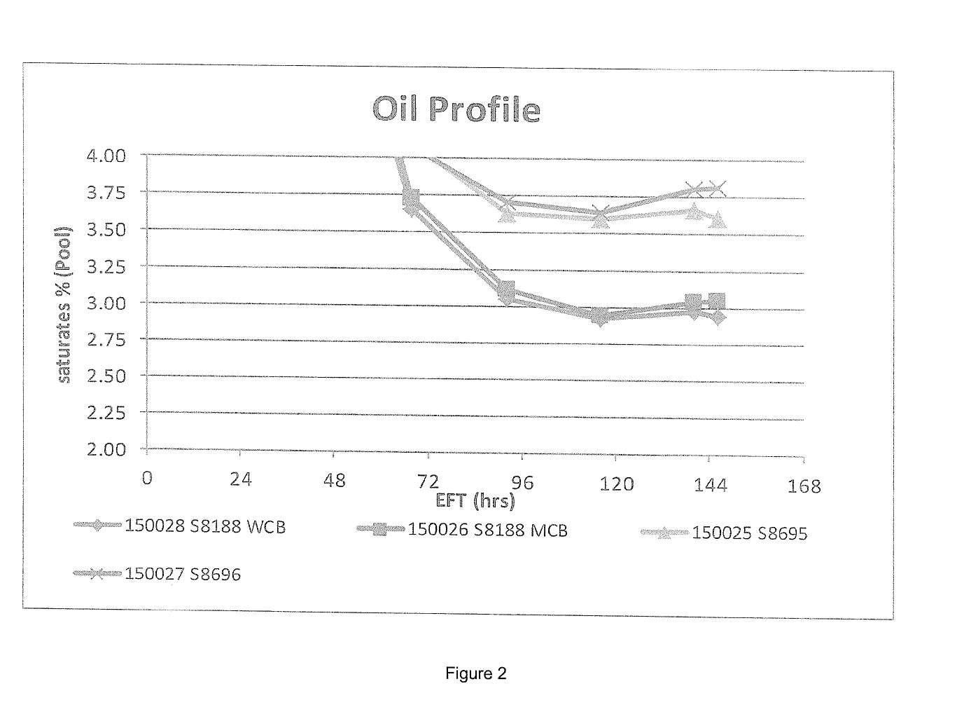

The resulting profiles from representative clones arising from transformations of pSZ4768 (D3870) into S7740 are shown in Table 56. The impact of overexpressing the PmSAD2-1 gene is a clear diminution of C18:0 chain lengths, thereby significantly reduced the level of total saturated fatty acids. Strain S8188 is one of the stable lines from the transformant D3870-21 (Table 56), and it produces ˜4% total saturated fatty acids when evaluated in shake flask experiment. To confirm that S8188 is able to produce oil with lower total saturates, the performance of S8188 was further evaluated in a fermentation experiment. As shown in FIG. 1, strain S8188 produces 2.9-3.0% total saturates in both fermentation runs 140558F22 and 140574F24.

TABLE 56
Fatty acid profile of representative clones arising from transformation
with D3870 (pSZ4768) DNA, into strain S7740.

| Sample ID | C16:0 | C18:0 | C18:1 | C18:2 |
|---|---|---|---|---|
| pH 5; S7740; T1089; D3870-20; | 2.51 | 0.88 | 86.59 | 7.26 |
| pH 5; S7740; T1089; D3870-13; | 2.50 | 1.09 | 88.55 | 5.41 |
| pH 5; S7740; T1089; D3870-21; | 2.89 | 1.25 | 89.03 | 4.55 |
| pH 5; S7740; T1089; D3870-24; | 2.16 | 1.67 | 89.38 | 4.39 |
| pH 5; S7740; T1089; D3870-8; | 2.18 | 1.74 | 88.62 | 5.04 |
| pH 5; S7740; T1089; D3870-17; | 2.37 | 1.75 | 88.44 | 4.94 |
| pH 5; S7740; | 2.56 | 5.15 | 82.59 | 6.31 |

### Example 10

### Expression of LPAAT in High-Erucic Transgenic Microalgae

In the below given example we demonstrate the feasibility of using lysophosphatidic acid acyltransferase (LPAAT) to alter the content and composition of oils in our transgenic algal strains for producing certain very long chain fatty acids (VLCFA). Specifically we show that expression of a heterologous LPAAT gene from Limnanthes douglasii (LimdLPAAT, Uniprot Accession No:Q42870, SEQ ID NO: 82) or Limnanthes alba (LimaLPAAT, Uniprot Accession No: 42868, SEQ ID NO: 83) in transgenic high-erucic strains S7211 and S7708 results in more than 3 fold enhancement in erucic (22:1^{Δ13}) acid content in individual lines over the parents. S7211 and S7708 were generated by expressing either genes encoding Crambe hispanica subsp. abyssinica (also called Crambe abyssinica) (SEQ ID NO: 84) and Lunaria annua (SEQ ID NO: 85) fatty acid elongase (FAE), respectively, as disclosed in co-owned application WO2013/158938 in classically mutagenized derivative of a pool of UTEX 1435 and S3150 (selected for high oil production).

In this example S7211 and S7708 strains, transformed with the construct pSZ5119, were generated which express Sacharomyces carlbergenesis MEL1 gene (allowing for their selection and growth on medium containing melibiose) and L. douglasii LPAAT gene targeted at endogenous PmLPAAT1-1 genomic region. Construct pSZ5119 introduced for expression in S7211 and S7708 can be written as LPAAT1-1 5′ flank::PmHXT1-ScarMEL1-CvNR:PmSAD2-2v2-LimdLPAAT-CvNR::LPAAT1-1 3′ flank.

The sequence of the transforming DNA is provided in SEQ ID NO: 104. Relevant restriction sites in the construct are indicated in lowercase, underlined bold, and are from 5′-3′ BspQI, KpnI, SpeI, SnaBI, EcoRI, SpeI, AflII, SacI, BspQI, respectively. BspQI sites delimit the 5′ and 3′ ends of the transforming DNA. Bold, lowercase sequences represent genomic DNA from S3150 that permit targeted integration at the PLSC-2/LPAAT1-1 locus via homologous recombination. Proceeding in the 5′ to 3′ direction, the endogenous P. moriformis Hexose Transporter 1 promoter driving the expression of the S. carlbergenesis MEL1 gene (encoding an alpha galactosidase enzyme activity required for catabolic conversion of Meliobise to glucose and galactose, thereby permitting the transformed strain to grow on melibiose) is indicated by lowercase, boxed text. The initiator ATG and terminator TGA for MEL1 are indicated by uppercase italics, while the coding region is indicated with lowercase italics. The Chlorella vulgaris nitrate reductase (NR) gene 3′ UTR is indicated by lowercase underlined text followed by an endogenous AMT3 promoter of P. moriformis, indicated by boxed italicized text. The Initiator ATG and terminator TGA codons of the LimdLPAAT are indicated by uppercase, bold italics, while the remainder of the gene is indicated by bold italics. The C. vulgaris nitrate reductase 3′ UTR is again indicated by lowercase underlined text followed by the S3150 PLSC-2/LPAAT1-1 genomic region indicated by bold, lowercase text. The final construct was sequenced to ensure correct reading frames and targeting sequences.

Construct Used for the Expression of the Limnanthes douglasii Lysophosphatidic Acid Acyltransferase (LimdLPAAT) in Erucic Strains S7211 and S7708—

Nucleotide sequence of transforming DNA contained in plasmid pSZ5119:
(SEQ ID NO: 104)
gctcttctgcttcggattccactacatcaagtgggtgaacctggcgggcgcggaggagggcccccgcccgggcggcat
tgttagcaaccactgcagctacctggacatcctgctgcacatgtccgattccttccccgcctttgtggcgcgccagtcga
cggccaagctgccctttatcggcatcatcaggtgcgtgaaagtgggggctgctgtggtcgtggtgggcggggtcacaa
atgaggacattgatgctgtcgtttgccgatcaggggagctcgaaagtaagtgcagcctggtcatgggatcacaaatct
caccaccactcgtccaccttgcctgggccttgcagccaaattatgagctgcctctacgtgaaccgcgaccgctcggggc
ccaaccacgtgggtgtggccgacctggtgaagcagcgcatgcaggacgaggccgaggggaagaccccgcccgagta
ccggccgctgctcctcttccccgaggtgggcttttgagacactgtttgtgcttgaaactgtggacgcgcgtgccctgacg
cgcctccggcgcctgtctcgcatccattcgcctctcaaccccatctcaccttttctccatcgccagggcaccacctccaac

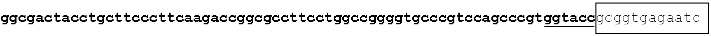

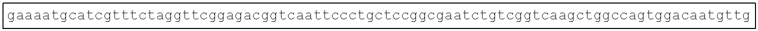

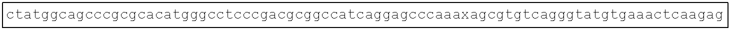

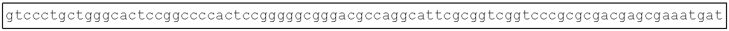

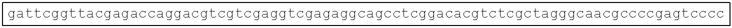

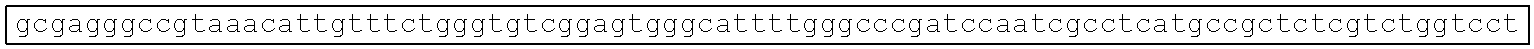

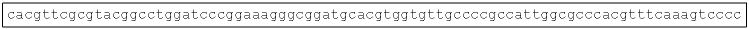

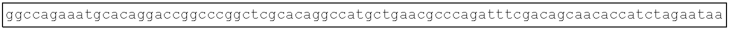

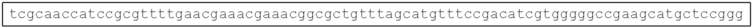

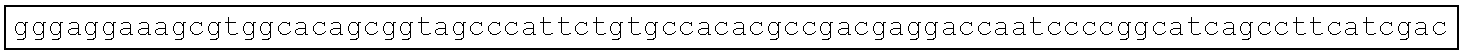

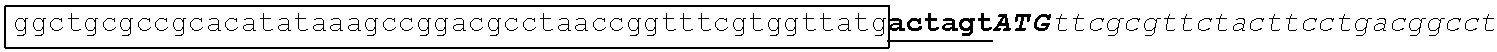

gcatctccctgaagggcgtgttcggcgtctccccctcctacaacggcctgggcctgacgccccagatgggctgggacaactg
gaacacgttcgcctgcgacgtctccgagcagctgctgctggacacggccgaccgcatctccgacctgggcctgaaggacat
gggctacaagtacatcatcctggacgactgctggtcctccggccgcgactccgacggcttcctggtcgccgacgagcagaa
gttccccaacggcatgggccacgtcgccgaccacctgcacaacaactccacctgacggcatgtactcctccgcgggcgag
tacacgtgcgccggctaccccggctccctgggccgcgaggaggaggacgcccagttcttcgcgaacaaccgcgtggacta
cctgaagtacgacaactgctacaacaagggccagttcggcacgcccgagatctcctaccaccgctacaaggccatgtccg
acgccctgaacaagacgggccgccccatcactactccctgtgcaactggggccaggacctgaccactactggggctccgg
catcgcgaactcctggcgcatgtccggcgacgtcacggcggagttcacgcgccccgactcccgctgcccctgcgacggcga
cgagtacgactgcaagtacgccggcaccactgctccatcatgaacatcctgaacaaggccgcccccatgggccagaacgc
gggcgtcggcggctggaacgacctggacaacctggaggtcggcgtcggcaacctgacggacgacgaggagaaggcgc
acttctccatgtgggccatggtgaagtcccccctgatcatcggcgcgaacgtgaacaacctgaaggcctcctcctactccatc
tactcccaggcgtccgtcatcgccatcaaccaggactccaacggcatccccgccacgcgcgtctggcgctactacgtgtccg
acacggacgagtacggccagggcgagatccagatgtggtccggccccctggacaacggcgaccaggtcgtggcgctgct
gaacggcggctccgtgtcccgccccatgaacacgaccctggaggagatcttcttcgactccaacctgggctccaagaagct
gacctccacctgggacatctacgacctgtgggcgaaccgcgtcgacaactccacggcgtccgccatcctgggccgcaacaa
gaccgccaccggcatcctgtacaacgccaccgagcagtcctacaaggacggcctgtccaagaacgacacccgcctgttcg
gccagaagatcggctccctgtcccccaacgcgatcctgaacacgaccgtccccgcccacggcatcgcgttctaccgcctgcg

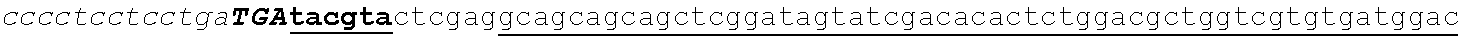

tgttgccgccacacttgctgccttgacctgtgaatatccctgccgcttttatcaaacagcctcagtgtgtttgatcttgtgtgtacgc
gcttttgcgagttgctagctgcttgtgctatttgcgaataccacccccagcatccccttccctcgtttcatatcgcttgcatcccaac
cgcaacttatctacgctgtcctgctatccctcagcgctgctcctgctcctgctcactgcccctcgcacagccttgattgggctccg
cctgtattctcctggtactgcaacctgtaaaccagcactgcaatgctgatgcacgggaagtagtgggatgggaacacaaatgga

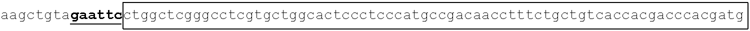

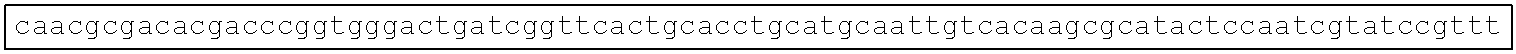

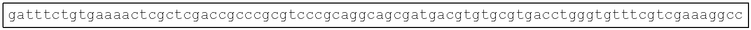

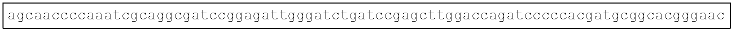

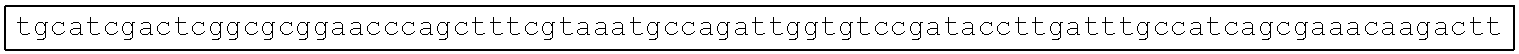

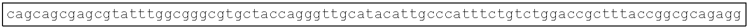

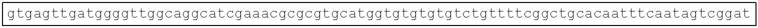

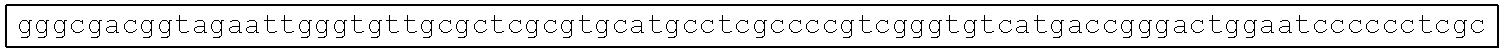

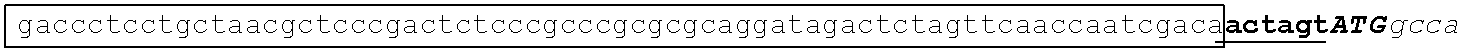

agacccgcacctcctccctgcgcaaccgccgccagctgaagcccgccgtggccgccaccgccgacgacgacaaggacggc
gtgttcatggtgctgctgtcctgcttcaagatcttcgtgtgcttcgccatcgtgctgatcaccgccgtggcctggggcctgatca
tggtgctgctgctgccctggccctacatgcgcatccgcctgggcaacctgtacggccacatcatcggcggcctggtgatctgg
atctacggcatccccatcaagatccagggctccgagcacaccaagaagcgcgccatctacatctccaaccacgcctccccc
atcgacgccttcttcgtgatgtggctggcccccatcggcaccgtgggcgtggccaagaaggaggtgatctggtaccccctgc
tgggccagctgtacaccctggcccaccacatccgcatcgaccgctccaaccccgccgccgccatccagtccatgaaggagg
ccgtgcgcgtgatcaccgagaagaacctgtccctgatcatgttccccgagggcacccgctcccgcgacggccgcctgctgcc
cttcaagaagggcttcgtgcacctggccctgcagtcccacctgcccatcgtgcccatgatcctgaccggcacccacctggcct
ggcgcaagggcaccttccgcgtgcgccccgtgcccatcaccgtgaagtacctgccccccatcaacaccgacgactggaccg
tggacaagatcgacgactacgtgaagatgatccacgacgtgtacgtgcgcaacctgcccgcctcccagaagcccctgggc

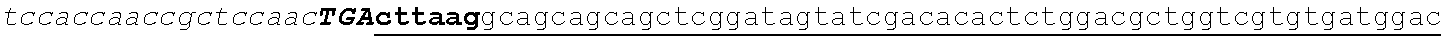

tgttgccgccacacttgctgccttgacctgtgaatatccctgccgcttttatcaaacagcctcagtgtgtttgatcttgtgtgtacgc
gcttttgcgagttgctagctgcttgtgctatttgcgaataccacccccagcatccccttccctcgtttcatatcgcttgcatcccaac
cgcaacttatctacgctgtcctgctatccctcagcgctgctcctgctcctgctcactgcccctcgcacagccttgattgggctccg
cctgtattctcctggtactgcaacctgtaaaccagcactgcaatgctgatgcacgggaagtagtgggatgggaacacaaatgga
aagcttaattaagagctccgtcctccactaccacagggtatggtcgtgtggggtcgagcgtgttgaagcgcagaagggg
atgcgccgtcaagatcaggagctaaaaatggtgccagcgaggatccagcgctctcactcttgctgccatcgctcccac
ccttttccccaggggaccctgtggcccacgtgggagacgattccggccaagtggcacatcttcctgatgctctgccaccc
ccgccacaaagtgaccgtgatgaaggttaggacaagggtcgggacccgattctggatatgacctctgaggtgtgtttct
cgcgcaagcgtcccccaattcgttacaccacatccctcacaccctcgcccctgacactcgcagttgcccgtgtacgtccc
caatgaggaggaaaaggccgaccccaagctgtacgcccaaaacgtccgcaaagccatggtgcgtcgggaaccgtca
aagtttgcttgcgggtgggcggggcggctctagcgaattggctcattggccctcaccgaggcagcacatcggacacca
gtcgccacccggcttgcatcttcgccccctttcttctcgcagatggaggtcgccgggaccaaggacacgacggcggtgt
ttgaggacaagatgcgctacctgaactccctgaagagaaagtacggcaagcctgtgcctaagaaaattgagtgaacc
cccgtcgtcgaccagaagagc

Constructs Used for the Expression of the LimdLPAAT and LimaLPAAT Genes from Higher Plants in S7211 and S7708.

In addition to the L. douglasii LPAAT targeted at PLSC-2/PmLPAAT1-1 locus (pSZ5119), L. douglasii LPAAT targeted at PLSC-2/LPAAT1-2 locus (pSZ5120), L. alba LPAAT targeted at PLSC-2/PmLPAAT1-1 locus (pSZ5343) and L. alba LPAAT targeted at PLSC-2/PmLPAAT1-2 locus (pSZ5348) have been constructed for expression in S7211 and S7708. These constructs can be described as:

pSZ5120: PLSC-2/LPAAT1-2 5′ flank::PmHXT1-ScarMEL1-CvNR:PmSAD2-2v2-LimdLPAAT-CvNR::PLSC-2/LPAAT1-2 3′ flank

pSZ5343: PLSC-2/LPAAT1-1 5′ flank::PmHXT1-ScarMEL1-CvNR:PmSAD2-2v2-LimaLPAAT-CvNR::PLSC-2/LPAAT1-1 3′ flank

pSZ5348: PLSC-2/LPAAT1-2 5′ flank::PmHXT1-ScarMEL1-CvNR:PmSAD2-2v2-LimaLPAAT-CvNR::PLSC-2/LPAAT1-2 3′ flank

All these constructs have the same vector backbone; selectable marker, promoters, and 3′ utr as pSZ5119, differing only in either the genomic region used for construct targeting and/or the respective LPAAT gene. Relevant restriction sites in these constructs are also the same as in pSZ5119. The sequences immediately below indicate the sequence of PLSC-2/LPAAT1-2 5′ flank, PLSC-2/LPAAT1-2 3′ flank, LimaLPAAT respectively. Relevant restriction sites as bold text are shown 5′-3′ respectively.

Sequence of PLSC-2/LPAAT1-2 5′ flank in pSZ5120 and pSZ5348
PLSC-2/LPAAT1-2 5′ flank:
(SEQ ID NO: 105)
gctcttctgcttcggattccactacatcaagtgggtgaacctggcgggcgcggaggagggcccccgcccgggcggcat
tgttagcaaccactgcagctacctggacatcctgctgcacatgtccgactccttccccgcctttgtggcgcgccagtcga
cggccaagctgccctttatcggcatcatcaggtgcgtgaaagcgggggctgctgtggccgtggtgggcagggttgcga
aggggggcaggcgtaggcgtgcagtgtgagcggacattgatgccgtcgtttgccggtcaggagagctcgaaatcaga
gccagcctggtcatgggatcacagagctcaccaccactcgtccacctcgcctgcgccttgcagccaaatcatgagctgc
ctctacgtgaaccgcgaccgctcggggcccaaccacgtgggcgtggccgatctggtgaagcagcgcatgcaggacga
ggccgaggggaggaccccgcccgagtaccgaccgctgctcctcttccccgaggtgggctttcgaggcaccgtttgtgct
tgaaactgtgggcacgcgtgccccgacgcgcctctggcgcctgcttcgcatccattcgcctctcaaccccgtctctccttt
cctccatcgccagggcaccacctccaacggcgactacctgcttcccttcaagaccggcgccttcctggccggggtgccc
gtccagcccgtggtacc
Sequence of PLSC-2/LPAAT1-2 3′ flank in pSZ5120 and pSZ5348
PLSC-2/LPAAT1-2 3′ flank:
(SEQ ID NO: 106)
gagctccgtcctccactaccacagggtatggtggtgtggggtcgagcgtgttgaagcgcggaaggggatgcgctgtca
agttttggagctgaaaatggtgcccgcgaggatccagcgcgccccactcacccttgctgccatcgctccccacccttttc
cccagggaaccctgtggcccacgtgggagacgattccggccaagtggcacatcttcctgatgctctgccacccccgcc
acaaagtgaccgtgatgaaggtacgaacaagggtcgggccccgattctggatatcacgtctggggtgtgtttctcgcg
cacgcgtcccccgatgcgctgcacagtctccctcacaccctcacccctaacgctcgcagttgcccgtgtacgtccccaat
gaggaggaaaaggccgaccccaagctgtacgcccaaaatgttcgcaaagccatggtgcgtcgggaaccgttcaagtt
tgcttgcgggtgggcggggcggctctagcgaattggcgcattggccctcaccgaggcagcacatcggacaccaatcgt
cacccggcgagcaattccgccccctctgtcttctcgcagatggaggtcgccgggaccaaggacacgacggcggtgttt
gaggacaagatgcgctacctgaactccctgaagagaaagtacggcaagcctgtgcctaagaaaattgagtgaacccc
cgtcgtcgaccagaagagc
Nucleotide sequence of L. alba LPAAT (LimaLPAAT) contained in pSZ5343 and
pSZ5348 - LimaLPAAT:
(SEQ ID NO: 107)

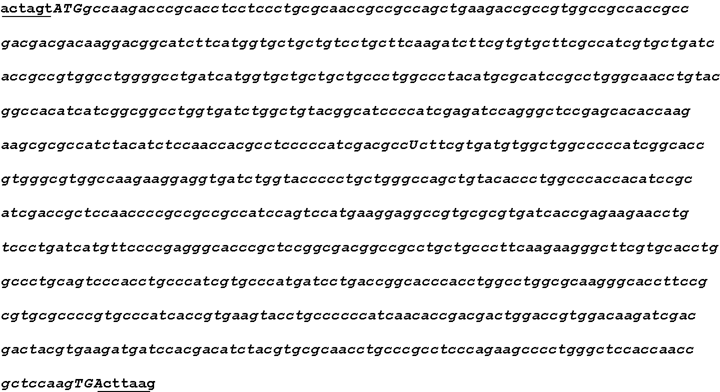

To determine their impact on fatty acid profiles, all the constructs described above were transformed independently into either S7211 or S7708. Primary transformants were clonally purified and grown under standard lipid production conditions at pH7.0. Strains S7211 and S7708 express a FAE, from C. abyssinica or L. annua respectively, under the control of pH regulated, AMT03 (Ammonium transporter 03) promoter. Thus both parental (S7211 and S7708) and the resulting LPAAT transformed strains require growth at pH 7.0 to allow for maximal fatty acid elongase (FAE) gene expression. The resulting profiles from a set of representative clones arising from transformations with pSZ5119 (D3979), pSZ5120 (D3980), pSZ5343 (D4204), and pSZ5348 (D4209) into S7211 or S7708 are shown in Tables 57-62.

All the transgenic S7211 or S7708 strains expressing LPAAT gene from either L. douglasii or L. alba show 2 fold or more enhanced accumulation of C22:1 fatty acid (see tables 57-62). The enhancement in erucic (C22:1^{Δ13}) acid levels is 4.2 fold in S7708; T1127; D3979-15 over the parent S7708 and 3.7 fold in S7211; T1181; D4204-5; pH7 over the parent S7211. These results clearly demonstrate using LPAAT genes to alter the VLCFA content in transgenic algal strains.

TABLE 57
Unsaturated fatty acid profile in S3150, S7211 and representative
derivative transgenic lines transformed with pSZ5119
(LimdLPAAT at PLSC-2/LPAAT1-1 genomic locus) DNA.

| Sample ID | C18:1 | C18:2 | C18:3a | Sum C20:1 | C22:1 |
|---|---|---|---|---|---|
| S7211; T1120; | 37.01 | 14.5 | 1.63 | 6.95 | 4.32 |
| D3979-24; pH 7 | | | | | |
| S7211; T1120; | 38.99 | 13.63 | 1.54 | 6.31 | 3.96 |
| D3979-31; pH 7 | | | | | |
| S7211; T1120; | 44.87 | 10.84 | 1.05 | 4.98 | 1.99 |
| D3979-2; pH 7 | | | | | |
| S7211; T1120; | 46.10 | 10.43 | 1.01 | 4.69 | 1.97 |
| D3979-19; pH 7 | | | | | |
| S7211; T1120; | 43.80 | 10.66 | 1.05 | 4.73 | 1.97 |
| D3979-29; pH 7 | | | | | |
| S7211A; pH 7 | 46.80 | 9.89 | 0.84 | 4.40 | 1.60 |
| S7211B; pH 7 | 46.80 | 9.89 | 0.84 | 4.37 | 1.65 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 58
Unsaturated fatty acid profile in S3150, S7211 and representative
derivative transgenic lines transformed with pSZ5120
(LimdLPAAT at PLSC-2/LPAAT1-1 genomic locus) DNA.

| Sample ID | C18:1 | C18:2 | C18:3a | C20:1 Sum | C22:1 |
|---|---|---|---|---|---|
| S7211; T1120; | 36.92 | 14.01 | 1.93 | 6.41 | 4.36 |
| D3980-45; pH 7 | | | | | |
| S7211; T1120; | 35.91 | 15.31 | 2.14 | 6.13 | 3.55 |
| D3980-48; pH 7 | | | | | |
| S7211; T1120; | 34.38 | 17.95 | 2.93 | 5.44 | 2.50 |
| D3980-27; pH 7 | | | | | |
| S7211; T1120; | 41.52 | 12.09 | 1.12 | 5.03 | 2.26 |
| D3980-46; pH 7 | | | | | |
| S7211; T1120; | 43.64 | 11.25 | 1.09 | 5.39 | 2.25 |
| D3980-14; pH 7 | | | | | |
| S7211A; pH 7 | 46.80 | 9.89 | 0.84 | 4.4 | 1.6 |
| S7211B; pH 7 | 46.80 | 9.89 | 0.84 | 4.37 | 1.65 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 59
Unsaturated fatty acid profile in S3150, S7708 and representative
derivative transgenic lines transformed with pSZ5119
(LimdLPAAT at PLSC-2/LPAAT1-2 genomic locus) DNA.

| Sample ID | C18:1 | C18:2 | C18:3a | Sum C20:1 | C22:1 |
|---|---|---|---|---|---|
| S7708; T1127; | 33.34 | 14.98 | 1.95 | 4.09 | 6.50 |
| D3979-15; pH 7 | | | | | |
| S7708; T1127; | 43.31 | 11.28 | 1.05 | 4.72 | 3.89 |
| D3979-32; pH 7 | | | | | |
| S7708; T1127; | 42.76 | 11.35 | 1.05 | 4.65 | 3.81 |
| D3979-42; pH 7 | | | | | |
| S7708; T1127; | 46.67 | 10.22 | 1.07 | 4.18 | 3.19 |
| D3979-3; pH 7 | | | | | |
| S7708; T1127; | 46.38 | 9.96 | 0.90 | 4.14 | 3.00 |
| D3979-40; pH 7 | | | | | |
| S7708A; pH 7 | 49.61 | 8.47 | 0.69 | 2.91 | 1.53 |
| S7708B; pH 7 | 50.14 | 8.37 | 0.70 | 2.97 | 1.52 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 60
Unsaturated fatty acid profile in S3150, S7708 and representative
derivative transgenic lines transformed with pSZ5120
(LimdLPAAT at PLSC-2/LPAAT1-2 genomic locus) DNA.

| Sample ID | C18:1 | C18:2 | C18:3a | Sum C20:1 | C22:1 |
|---|---|---|---|---|---|
| S7708; T1127; | 44.49 | 12.25 | 1.41 | 5.14 | 3.80 |
| D3980-24; pH 7 | | | | | |
| S7708; T1127; | 46.89 | 9.97 | 0.93 | 4.40 | 2.66 |
| D3980-42; pH 7 | | | | | |
| S7708; T1127; | 47.77 | 10.08 | 0.91 | 4.21 | 2.44 |
| D3980-43; pH 7 | | | | | |
| S7708; T1127; | 50.36 | 8.80 | 0.68 | 3.61 | 2.13 |
| D3980-14; pH 7 | | | | | |
| S7708; T1127; | 47.55 | 10.49 | 0.64 | 3.64 | 2.13 |
| D3980-17; pH 7 | | | | | |
| S7708A; pH 7 | 49.61 | 8.47 | 0.69 | 2.91 | 1.53 |
| S7708B; pH 7 | 50.14 | 8.37 | 0.7 | 2.97 | 1.52 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 61
Unsaturated fatty acid profile in S3150, S7708 and representative
derivative transgenic lines transformed with pSZ5343
(LimaLPAAT at PLSC-2/LPAAT1-1 genomic locus) DNA.

| Sample ID | C18:1 | C18:2 | C18:3a | Sum C20:1 | C22:1 |
|---|---|---|---|---|---|
| S7211; T1181; | 37.27 | 13.62 | 1.60 | 6.64 | 5.12 |
| D4204-5; pH 7 | | | | | |
| S7211; T1181; | 39.39 | 12.58 | 1.78 | 5.86 | 3.12 |
| D4204-16; pH 7 | | | | | |
| S7211; T1181; | 42.52 | 11.53 | 1.31 | 4.82 | 2.01 |
| D4204-6; pH 7 | | | | | |
| S7211; T1181; | 45.97 | 10.56 | 0.99 | 4.73 | 1.92 |
| D4204-2; pH 7 | | | | | |
| S7211; T1181; | 45.76 | 10.52 | 1.00 | 4.63 | 1.88 |
| D4204-11; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 57.99 | 6.62 | 0.56 | 0.19 | 0 |
| S3150; pH 5 | 57.7 | 7.08 | 0.54 | 0.11 | 0 |

TABLE 62
Unsaturated fatty acid profile in S3150, S7708 and representative
derivative transgenic lines transformed with pSZ5348
(LimaLPAAT at PLSC-2/LPAAT1-2 genomic locus) DNA.

| Sample ID | C18:1 | C18:2 | C18:3a | Sum C20:1 | C22:1 |
|---|---|---|---|---|---|
| S7211; T1181; | 40.46 | 13.18 | 1.43 | 6.59 | 3.94 |
| D4209-24; pH 7 | | | | | |
| S7211; T1181; | 41.79 | 12.71 | 1.29 | 6.10 | 3.50 |
| D4209-18; pH 7 | | | | | |
| S7211; T1181; | 43.32 | 11.65 | 1.45 | 5.22 | 2.79 |
| D4209-3; pH 7 | | | | | |
| S7211; T1181; | 47.41 | 9.68 | 1.01 | 6.01 | 2.36 |
| D4209-27; pH 7 | | | | | |
| S7211; T1181; | 43.67 | 12.77 | 0.99 | 5.05 | 2.24 |
| D4209-5; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 57.99 | 6.62 | 0.56 | 0.19 | 0 |
| S3150; pH 5 | 57.70 | 7.08 | 0.54 | 0.11 | 0 |

### Example 11

### Expression of LPCAT in a Microalga

Here we demonstrate the feasibility of using higher plant Lysophosphatidylcholine acyltransferase (LPCAT) genes to alter the content and composition of oils in transgenic algal strains for producing oils rich in linoleic acid. We demonstrate that expression of heterologous LPCAT enzymes in P. moriformis strain S7485 results in more than 3 fold enhancement in linoleic (C18:2) acid in individual lines over the parents.

Wildtype Prototheca strains when cultured under low-nitrogen lipid production conditions result in extracted cell oil with around 5-7% C18:2 levels and point towards a functional endogenous LPCAT and downstream DAG-CPT and/or PDCT enzyme in our host. When higher plant LPCATs or DAG-CPTs are used as baits, transcripts for both genes were found the P. moriformis transcriptome. However no hits for a corresponding PDCT like gene were found.

We have identified both alleles of LPCAT in Prototheca moriformis (PmLPCAT1). The overall transcription of both alleles is very low. Transcript levels for both start out at 50-60 transcripts per million and then slowly increase over the course of lipid production. PmLPCAT1-1 reaches around 210 transcripts per million while PmLPCAT1-2 increases to around 150 transcripts per million

Two LPCAT genes from A. thaliana encoding (AtLPCAT1 NP_172724.2 [SEQ ID NO: 86], AtLPCAT2 NP_176493.1[SEQ ID NO: 87]) available in the public databases were used to identify corresponding LPCAT genes from our internally assembled transcriptomes of B. rapa, B. juncea and L. douglasii. 5 full-length sequences were identified and named as BrLPCAT [SEQ ID NO: 99], BjLPCAT1 [SEQ ID NO: 108], BjLPCAT2 [SEQ ID NO: 109], LimdLPCAT1 [SEQ ID NO: 101], and LimdLPCAT2 [SEQ ID NO: 102]. The codon optimized sequences of these enzymes except BjLPCAT1, along with the AtLPCAT genes, were expressed in P. moriformis strain S7485. S7485 is a strain made according to the methods disclosed in co-owned application No. 62/141,167 filed on 31 Mar. 2015. Specifically, S7485 is a cerulenin resistant isolate of Strain K with low C16:0 titer and high C18:1.

Construct Used for the Expression of the B. juncea Lysophosphatidylcholine Acyltransferase-1 (BjLPCAT1) in S7485 [pSZ5298]:

Strain S7485 was transformed with the construct pSZ5298, to express the Sacharomyces carlbergenesis MEL1 gene (allowing for their selection and growth on medium containing melibiose) and B. rapa LPCAT gene targeted at endogenous PmLPAAT1-1 genomic region. Construct pSZ5298 introduced for expression in S7485 can be written as PLSC-2/LPAAT1-1 5′ flank::PmHXT1-ScarMEL1-CvNR:PmSAD2-2v2-BjLPCAT1-CvNR:: PLSC-2/LPAAT1-1 3′ flank.

The sequence of the transforming DNA is provided below as SEQ ID NO: 110. Relevant restriction sites in the construct are indicated in lowercase, underlined bold, and are from 5′-3′ BspQI, KpnI, SpeI, SnaBI, EcoRI, SpeI, AflII, SacI, BspQI, respectively. BspQI sites delimit the 5′ and 3′ ends of the transforming DNA. Bold, lowercase sequences represent genomic DNA from S3150 that permit targeted integration at the PLSC-2/LPAAT1-1 locus via homologous recombination. Proceeding in the 5′ to 3′ direction, the endogenous P. moriformis Hexose Transporter 1 promoter driving the expression of the S. carlbergenesis MEL1 gene (encoding an alpha galactosidase enzyme activity required for catabolic conversion of Melibiose to glucose and galactose, thereby permitting the transformed strain to grow on melibiose) is indicated by lowercase, boxed text. The initiator ATG and terminator TGA for MEL1 are indicated by uppercase italics, while the coding region is indicated with lowercase italics. The Chlorella vulgaris nitrate reductase (NR) gene 3′ UTR is indicated by lowercase underlined text followed by an endogenous AMTS promoter of P. moriformis, indicated by boxed italicized text. The Initiator ATG and terminator TGA codons of the BjLPCAT1 are indicated by uppercase, bold italics, while the remainder of the gene is indicated by bold italics. The C. vulgaris nitrate reductase 3′ UTR is again indicated by lowercase underlined text followed by the S3150 PLSC-2/LPAAT1-1 genomic region indicated by bold, lowercase text. The final construct was sequenced to ensure correct reading frames and targeting sequences.

Nucleotide sequence of transforming DNA contained in plasmid pSZ5298:
(SEQ ID NO: 110)
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccattatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg

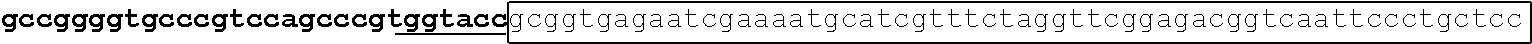

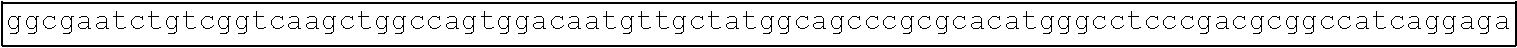

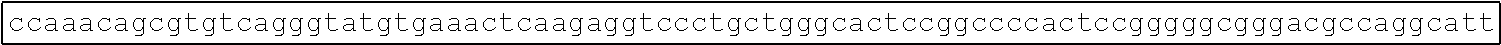

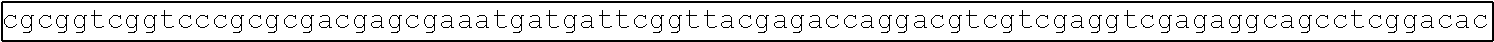

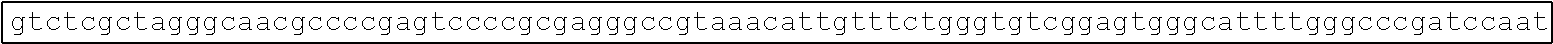

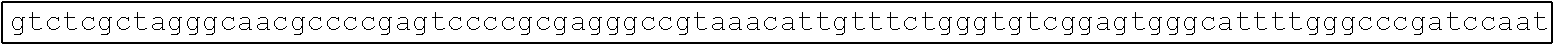

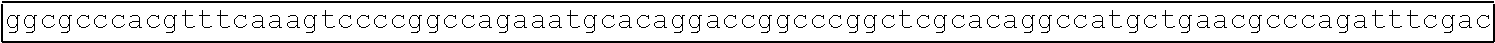

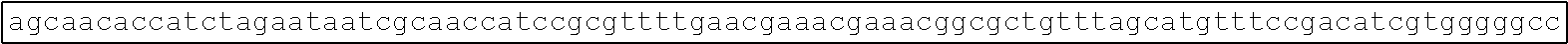

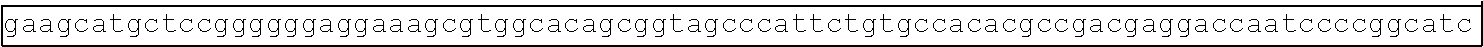

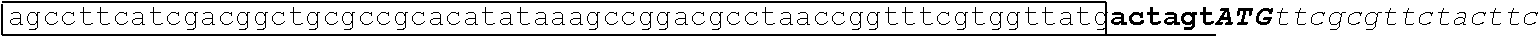

tgacggcctgcatctccctgaagggcgtgttcggcgtctccccctcctacaacggcctgggcctgacgccccagatgggctggga
caactggaacacgttcgcctgcgacgtctccgagcagctgctgctggacacggccgaccgcatctccgacctgggcctgaagga
catgggctacaagtacatcatcctggacgactgctggtcctccggccgcgactccgacggcttcctggtcgccgacgagcagaag
ttccccaacggcatgggccacgtcgccgaccacctgcacaacaactccttcctgttcggcatgtactcctgcggggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttaccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgttcggccagaagatcggctccctgtcccccaacgcgatcctgaacacg

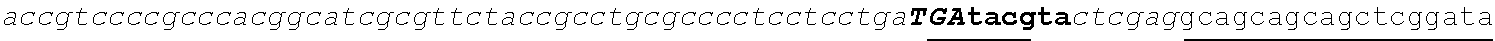

gtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtggg

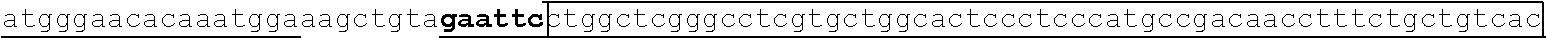

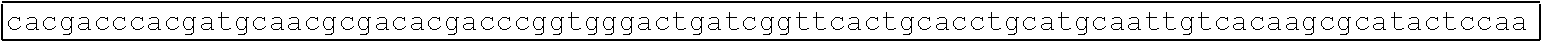

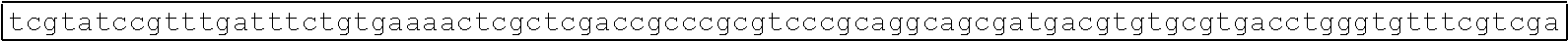

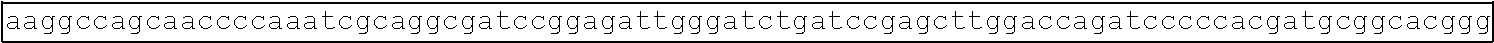

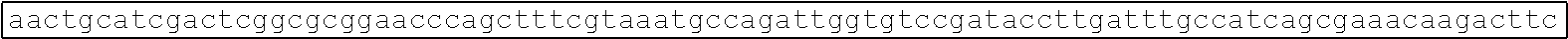

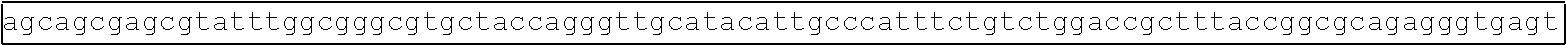

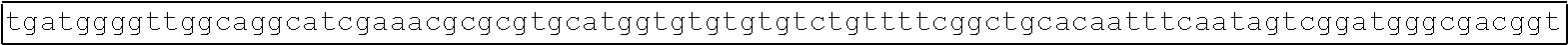

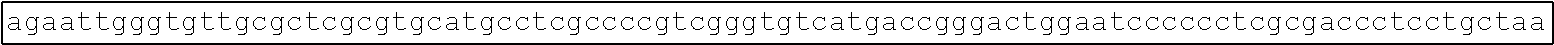

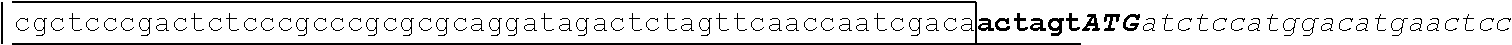

atggccgcctccatcggcgtgtccgtggccgtgctgcgcttcctgctgtgcttcgtggccaccatccccgtgtccttcgcctggcgcat
cgtgccctcccgcctgggcaagcacatctacgccgccgcctccggcgtgttcctgtcctacctgtccttcggcttctcctccaacctgc
acttcctggtgcccatgaccatcggctacgcctccatggccatgtaccgccccaagtgcggcatcatcaccttcttcctgggcttcgc
ctacctgatcggctgccacgtgttctacatgtccggcgacgcctggaaggagggcggcatcgactccaccggcgccctgatggtg
ctgaccctgaaggtgatctcctgcgccgtgaactacaacgacggcatgctgaaggaggagggcctgcgcgaggcccagaaga
agaaccgcctgatccagatgccctccctgatcgagtacttcggctactgcctgtgctgcggctcccacttcgccggccccgtgtacg
agatgaaggactacctgcagtggaccgagggcaagggcatctgggactcctccgagaagcgcaagcagccctccccctacgg
cgccaccctgcgcgccatcttccaggccggcatctgcatggccctgtacctgtacctggtgccccagttccccctgacccgcttcac
cgagcccgtgtaccaggagtggggcttcctgaagaagttcggctaccagtacatggccggccagaccgcccgctggaagtacta
cttcatctggtccatctccgaggcctccatcatcatctccggcctgggcttctccggctggaccgacgacgacgcctcccccaagcc
caagtgggaccgcgccaagaacgtggacatcctgggcgtggagctggccaagtccgccgtgcagatccccctggtgtggaaca
tccaggtgtccacctggctgcgccactacgtgtacgagcgcctggtgaagtccggcaagaaggccggcttcttccagctgctggcc
acccagaccgtgtccgccgtgtggcacggcctgtaccccggctacatgatgttcttcgtgcagtccgccctgatgatcgccggctcc
cgcgtgatctaccgctggcagcaggccatctcccccaagctggccatgctgcgcaacatcatggtgttcatcaacttcctgtacacc
gtgctggtgctgaactactccgccgtgggcttcatggtgctgtccctgcacgagaccctgaccgcctacggctccgtgtactacatc
ggcaccatcatccccgtgggcctgatcctgctgtcctacgtggtgcccgccaagccctcccgccccaagccccgcaaggaggag

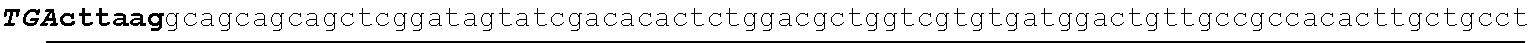

tgacctgtgaatatccctgccgcttttatcaaacagcctcagtgtgtttgatcttgtgtgtacgcgcttttgcgagttgctagctgcttgtgct
atttgcgaataccacccccagcatccccttccctcgtttcatatcgcttgcatcccaaccgcaacttatctacgctgtcctgctatccctcag
cgctgctcctgctcctgctcactgcccctcgcacagccttggtttgggctccgcctgtattctcctggtactgcaacctgtaaaccagcac
tgcaatgctgatgcacgggaagtagtgggatgggaacacaaatggaaagcttaattaagagctccgtcctccactaccacagggta
tggtcgtgtggggtcgagcgtgttgaagcgcagaaggggatgcgccgtcaagatcaggagctaaaaatggtgccagcgagg
atccagcgctctcactcttgctgccatcgctcccacccttttccccaggggaccctgtggcccacgtgggagacgattccggcca
agtggcacatcttcctgatgctctgccacccccgccacaaagtgaccgtgatgaaggttaggacaagggtcgggacccgattc
tggatatgacctctgaggtgtgtttctcgcgcaagcgtcccccaattcgttacaccacatccctcacaccctcgcccctgacactc
gcagttgcccgtgtacgtccccaatgaggaggaaaaggccgaccccaagctgtacgcccaaaacgtccgcaaagccatggt
gcgtcgggaaccgtcaaagtttgcttgcgggtgggcggggcggctctagcgaattggctcattggccctcaccgaggcagcac
atcggacaccagtcgccacccggcttgcatcttcgccccctttcttctcgcagatggaggtcgccgggaccaaggacacgacg
gcggtgtttgaggacaagatgcgctacctgaactccctgaagagaaagtacggcaagcctgtgcctaagaaaattgagtgaa
cccccgtcgtcgaccagaagagc

Constructs Used for the Expression of BrLPCAT, LimdLPCAT1, LimdLPCAT2, AtLPCAT1 and AtLPCAT2 Genes from Higher Plants in S7485.

In addition to the B. juncea LPCAT1 targeted at PLSC-2/PmLPAAT1-1 locus (pSZ5298), B. rapa LPCAT targeted at PLSC-2/PmLPAAT1-1 locus (pSZ5299), L. douglasii LPCAT1 targeted at PLSC-2/PmLPAAT1-1 locus (pSZ5300), L. douglasii LPCAT2 targeted at PLSC-2/PmLPAAT1-1 locus (pSZ5301), A. thaliana LPCAT1 targeted at PLSC-2/LPAAT1-2 locus (pSZ5307), A. thaliana LPCAT2 targeted at PLSC-2/LPAAT1-2 locus (pSZ5308), B. rapa LPCAT targeted at PLSC-2/PmLPAAT1-2 locus (pSZ5309) and L. douglasii LPCAT2 targeted at PLSC-2/PmLPAAT1-2 locus (pSZ5310) have been constructed for expression in S7211. These constructs can be described as:
pSZ5299: PLSC-2/LPAAT1-1::PmHXT1-ScarMEL1-CvNR:PmSAD2-2v2-BrLPCAT-CvNR::PLSC-2/LPAAT1-1
pSZ5300: PLSC-2/LPAAT1-1::PmHXT1-ScarMEL1-CvNR:PmSAD2-2v2-LimdLPCAT1-CvNR::PLSC-2/LPAAT1-1
pSZ5301: PLSC-2/LPAAT11::PmHXT1-ScarMEL1-CvNR:PmSAD2-2v2-LimdLPCAT2-CvNR::PLSC-2/LPAAT1-1
pSZ5307: PLSC-2/LPAAT1-2::PmHXT1-ScarMEL1-CvNR:PmSAD2-2v2-AtLPCAT1-CvNR::PLSC-2/LPAAT1-2
pSZ5308: PLSC-2/LPAAT1-2::PmHXT1-ScarMEL1-CvNR:PmSAD2-2v2-AtLPCAT2-CvNR::PLSC-2/LPAAT1-2
pSZ5309: PLSC-2/LPAAT1-2::PmHXT1-ScarMEL1-CvNR:PmSAD2-2v2-BrLPCAT-CvNR::PLSC-2/LPAAT1-2
pSZ5310: PLSC-2/LPAAT1 2::PmHXT1-ScarMEL1-CvNR:PmSAD2-2v2-LimdLPCAT2-CvNR::PLSC-2/LPAAT1-2

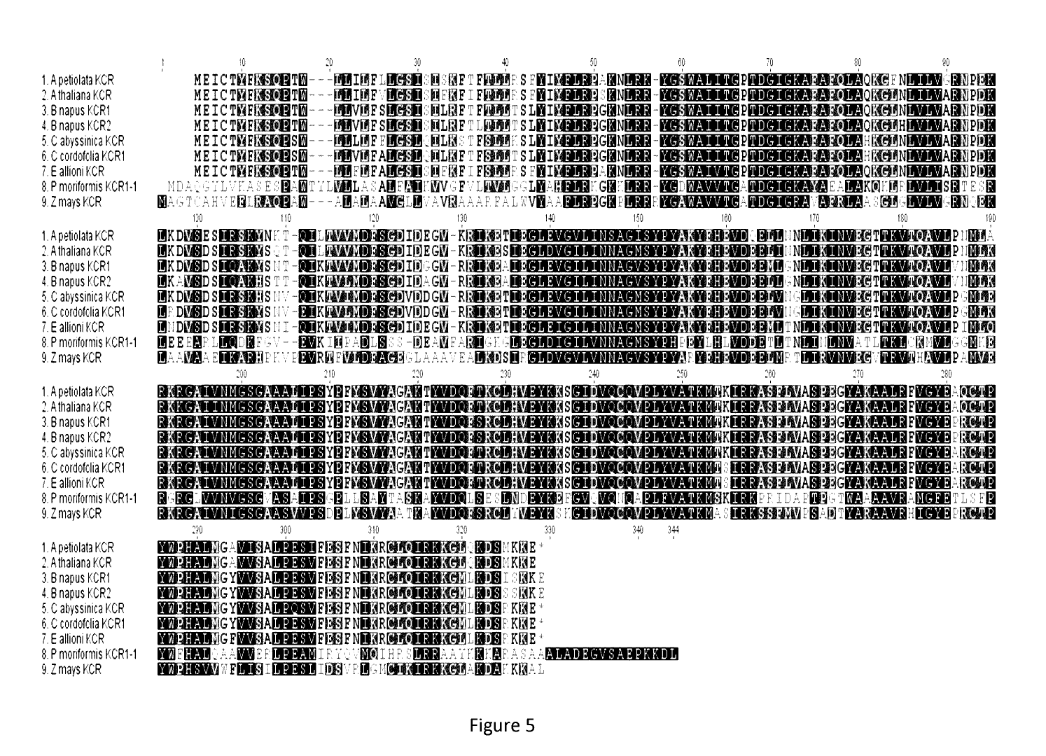

All these constructs have the same vector backbone; selectable marker, promoters, and 3′ utr as pSZ5298, differing only in either the genomic region used for construct targeting and/or the respective LPCAT gene. Relevant restriction sites in these constructs are also the same as in pSZ5298. FIGS. 5-11 indicate the sequence of PLSC-2/LPAAT1-2 5′ flank, PLSC-2/LPAAT1-2 3′ flank, BrLPCAT, LimdLPCAT1, LimdLPCAT2, AtLPCAT1 and AtLPCAT2 respectively. Relevant restriction sites as bold text are shown 5′-3′ respectively.

Sequence of PLSC-2/LPAAT1-2 5′ flank in pSZ5307, pSZ5308, pSZ5309, and
pSZ5310. PLS C-2/LPAAT1 -2 5′ flank:
(SEQ ID NO: 111)
gctcttctgcttcggattccactacatcaagtgggtgaacctggcgggcgcggaggagggcccccgcccgggcggcattgtta
gcaaccactgcagctacctggacatcctgctgcacatgtccgactccttccccgcctttgtggcgcgccagtcgacggccaagc
tgccctttatcggcatcatcaggtgcgtgaaagcgggggctgctgtggccgtggtgggcagggttgcgaaggggggcaggcg
taggcgtgcagtgtgagcggacattgatgccgtcgtttgccggtcaggagagctcgaaatcagagccagcctggtcatgggat
cacagagctcaccaccactcgtccacctcgcctgcgccttgcagccaaatcatgagctgcctctacgtgaaccgcgaccgctc
ggggcccaaccacgtgggcgtggccgatctggtgaagcagcgcatgcaggacgaggccgaggggaggaccccgcccgagt
accgaccgctgctcctcttccccgaggtgggctttcgaggcaccgtttgtgcttgaaactgtgggcacgcgtgccccgacgcgc
ctctggcgcctgcttcgcatccattcgcctctcaaccccgtctctcctttcctccatcgccagggcaccacctccaacggcgacta
cctgcttcccttcaagaccggcgccttcctggccggggtgcccgtccagcccgtggtacc
Sequence of PLSC-2/LPAAT1-2 3′ flank in pSZ5307, pSZ5308, pSZ5309, and
pSZ5310. PLS C-2/LPAAT1 -2 3′ flank:
(SEQ ID NO: 112)
gagctccgtcctccactaccacagggtatggtggtgtggggtcgagcgtgttgaagcgcggaaggggatgcgctgtcaagttt
tggagctgaaaatggtgcccgcgaggatccagcgcgccccactcacccttgctgccatcgctccccacccttttccccagggaa
ccctgtggcccacgtgggagacgattccggccaagtggcacatcttcctgatgctctgccacccccgccacaaagtgaccgtg
atgaaggtacgaacaagggtcgggccccgattctggatatcacgtctggggtgtglltctcgcgcacgcgtcccccgatgcgct
gcacagtctccctcacaccctcacccctaacgctcgcagttgcccgtgtacgtccccaatgaggaggaaaaggccgaccccaa
gctgtacgcccaaaatgttcgcaaagccatggtgcgtcgggaaccgttcaagtttgcttgcgggtgggcggggcggctctagc
gaattggcgcattggccctcaccgaggcagcacatcggacaccaatcgtcacccggcgagcaattccgccccctctgtcttctc
gcagatggaggtcgccgggaccaaggacacgacggcggtgtttgaggacaagatgcgctacctgaactccctgaagagaaa
gtacggcaagcctgtgcctaagaaaattgagtgaacccccgtcgtcgaccagaagagc
Nucleotide sequence of B. rapa LPCAT (BrLPCAT) contained in pSZ5299 and
pSZ5309. BrLPCAT:
(SEQ ID NO: 112)

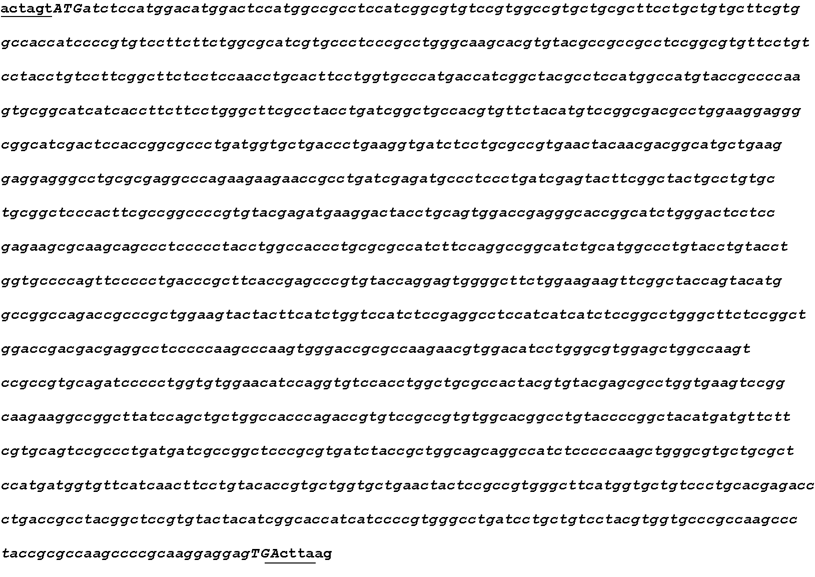

Nucleotide sequence of L. douglasii LPCATI (LimdLPCAT1) contained in
pSZ5300. LimdLPCAT1:
(SEQ ID NO: 113)

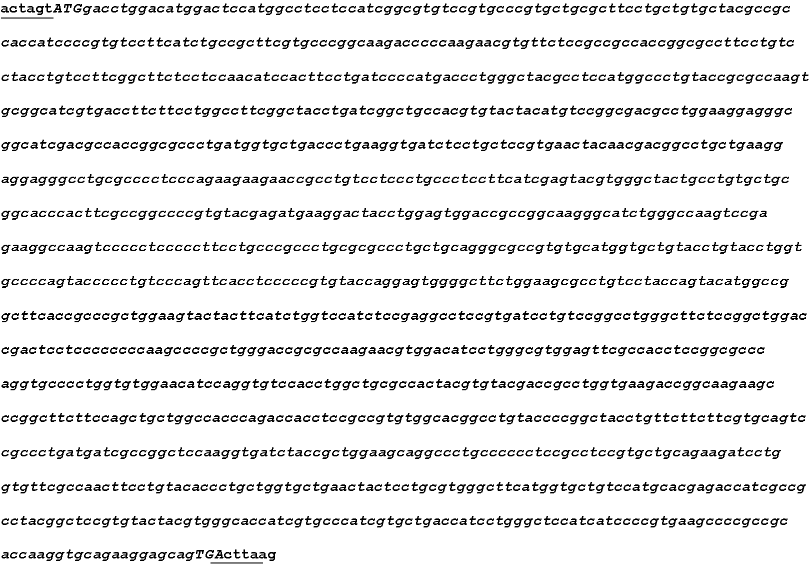

Nucleotide sequence of L. douglasii LPCAT2 (LimdLPCAT2) contained in
pSZ5301 and pSZ5310. LimdLPCAT2:
(SEQ ID NO: 114)

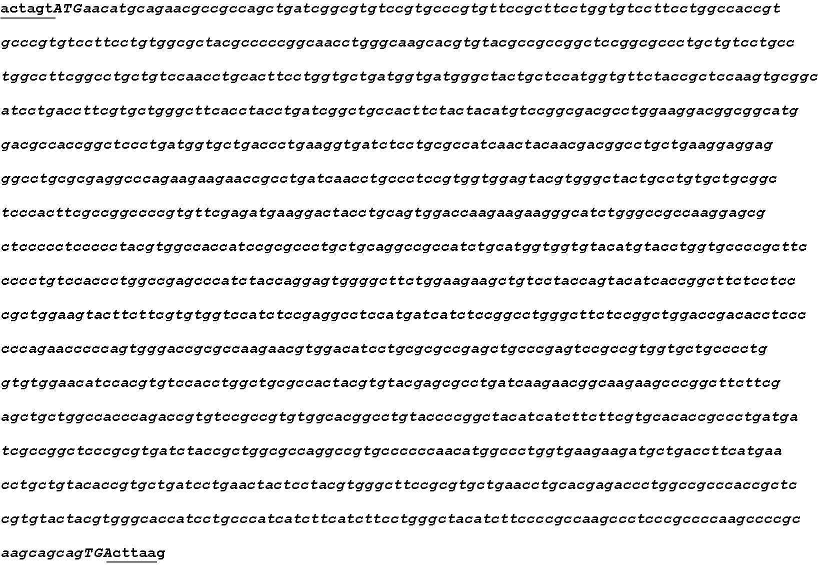

Nucleotide sequence of A. thaliana LPCAT1 (AtLPCAT1) contained in
pSZ5307. AtLPCAT1:
(SEQ ID NO: 115)

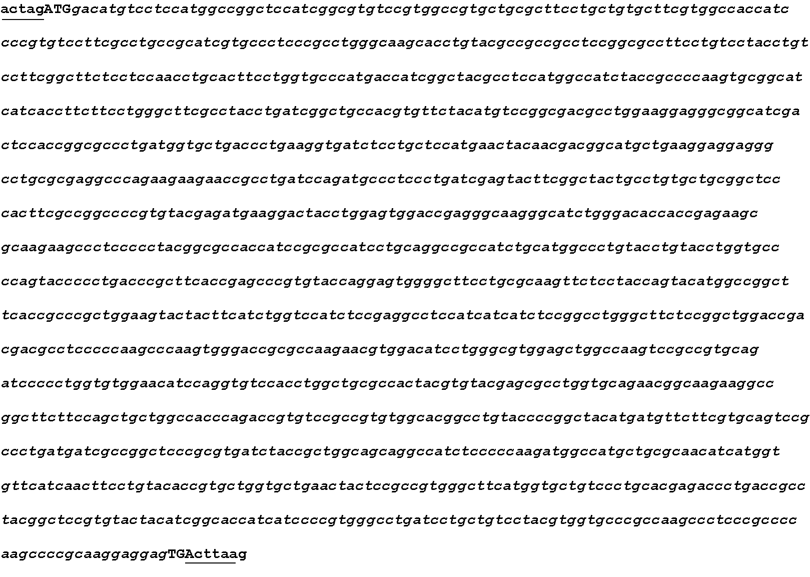

Nucleotide sequence of A. thaliana LPCAT 2 (AtLPCAT2) contained in
pSZ5308. AtLPCAT2:
(SEQ ID NO: 116)

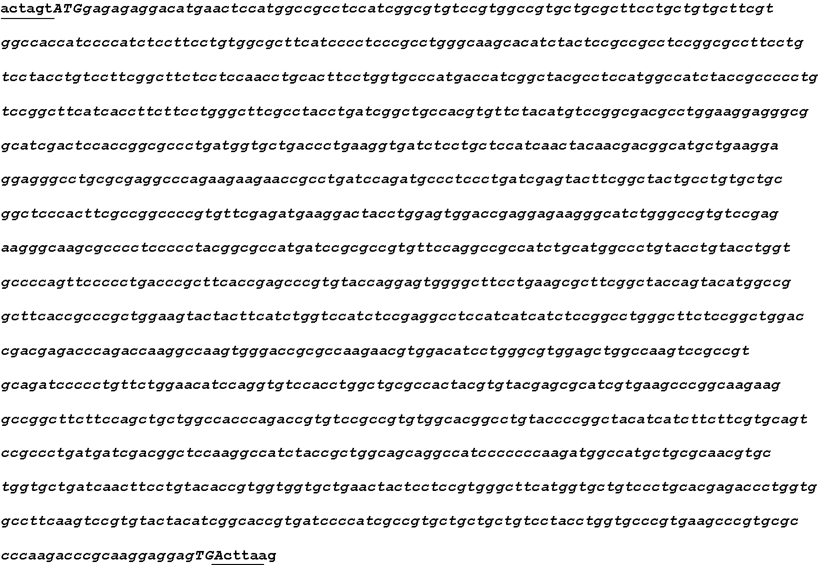

To determine their impact on fatty acid profiles, all the constructs described above were transformed independently into S7211. Primary transformants were clonally purified and grown under standard lipid production conditions at pH7.0. S7211 expresses a FAE, from C. abyssinica under the control of pH regulated, AMT03 (Ammonium transporter 03) promoter. Thus both parental (S7211) and the resulting LPCAT transformed strains require growth at pH 7.0 to allow for maximal fatty acid elongase (FAE) gene expression. The resulting profiles from a set of representative clones arising from transformations with pSZ5298 (D4159), pSZ5299 (D4160), pSZ5300 (D4161), pSZ5301 (D4162), pSZ5307 (D4168), pSZ5308 (D4169), pSZ5309 (D4170) and pSZ5310 (D4171) are shown in tables 63-70 respectively.

Except for L. douglasii LPCAT2, all the tested LPCAT enzymes resulted in 3 fold increase in C18:2 levels over the parent S7485. In the case of lines expressing LimdLPCAT2 increase in C18:2, while significant, was only 2 fold over the parent. The increase in C18:2 in S7211; T1172; D4157-14; pH7, expressing AtLPCAT1 at PLSC-2/LPAAT1-1 locus, was 2.54 fold (over parent S7211). These results strongly suggest that heterologous LPCAT gene expression in our algal host enhances the conversion of C18:1-CoA into C18:1-PC. The PC associated C18:1 is subsequently acted upon by downstream enzymes like FAD2 and converted into C18:2. As discussed above similar results were obtained when LPCAT genes were transformed into erucic strain S7211 (expressing CrhFAE). In S7211, gains in C18:2 levels were also associated with increases in erucic acid content. The combined results from both experiments suggest that most likely the CrhFAE in S7211 uses C18:1-PC rather than C18:1-CoA as a substrate for elongation. In this scenario PmFAD2 and CrhFAE in S7211 would compete for the same substrate resulting in elevated C18:2 as well as VLCFA like C20:1 and C22:1. If our hypothesis is correct then currently it would seem that PmFAD2-1 competes better for the substrate than CrhFAE. One of the approaches currently being pursued to channel more substrate for elongation is to reduce the PmFAD2 activity using RNAi Technology.

This example describes a significant increase in the C18:2 and C22:1 levels in an engineered microalgae.

Identification of LPCAT enzymes to increase conversion of C18:1 to C18:1-PC gives us a much better control over C18:1 phospholipid pool which can then be either directed towards making more polyunsaturated fatty acids or VLCFA by modulating the PmFAD2-1 activity.

TABLE 63
Unsaturated fatty acid profile in S7485 and representative derivative
transgenic lines transformed with pSZ5298 (BjLPCAT2) at PLSC-2/
LPAAT1-1 genomic locus) DNA.

| Sample ID | 14:0 | 16:0 | 18:0 | 18:1 | 18:2 | 18:3a |
|---|---|---|---|---|---|---|
| S7485 ctrl; pH 5 | .15 | 7.16 | .72 | 9.63 | .91 | .56 |
| S7485 ctrl; pH 5 | .18 | 7.24 | .74 | 9.45 | .94 | .57 |
| S7485; T1208; D4159-1; pH 5 | .27 | 7.48 | .87 | 0.42 | 3.61 | .60 |
| S7485; T1208; D4159-41; | .22 | 8.43 | .41 | 0.60 | 3.04 | .57 |
| pH 5 | | | | | | |
| S7485; T1208; D4159-24; | .43 | 0.10 | .82 | 8.98 | 2.82 | .81 |
| pH 5 | | | | | | |
| S7485; T1208; D4159-23; | .73 | 2.64 | .26 | 7.35 | 2.41 | .94 |
| pH 5 | | | | | | |
| S7485; T1208; D4159-18; | .08 | 7.47 | .66 | 2.42 | 2.16 | .53 |
| pH 5 | | | | | | |

TABLE 64
Unsaturated fatty acid profile in S7485 and representative derivative
transgenic lines transformed with pSZ5299 (BrLPCAT) at PLSC-2/
LPAAT1-1 genomic locus) DNA.

| Sample ID | 14:0 | 16:0 | 18:0 | 18:1 | 18:2 | 18:3a |
|---|---|---|---|---|---|---|
| S7485 ctrl; pH 5 | .15 | 7.16 | .72 | 9.63 | .91 | .56 |
| S7485 ctrl; pH 5 | .18 | 7.24 | .74 | 9.45 | .94 | .57 |
| S7485; T1208; D4160-44; | .50 | 0.23 | .51 | 0.06 | 2.60 | .54 |
| pH 5 | | | | | | |
| S7485; T1208; D4160-5; pH 5 | .27 | 8.69 | .78 | 1.45 | 2.25 | .70 |
| S7485; T1208; D4160-35; | .18 | 7.45 | .75 | 2.79 | 1.66 | .53 |
| pH 5 | | | | | | |
| S7485; T1208; D4160-30; | .20 | 7.66 | .72 | 2.65 | 1.60 | .54 |
| pH 5 | | | | | | |
| S7485; T1208; D4160-3; pH 5 | .12 | 7.26 | .77 | 3.08 | 1.59 | .55 |

TABLE 65
Unsaturated fatty acid profile in S7485 and representative derivative
transgenic lines transformed with pSZ5300 (LimdLPCAT1) at PLSC-2/
LPAAT1-1 genomic locus) DNA.

| Sample ID | 14:0 | 16:0 | 18:0 | 18:1 | 18:2 | 18:3a |
|---|---|---|---|---|---|---|
| S7485 ctrl; pH 5 | .15 | 7.14 | .72 | 9.62 | .94 | .58 |
| S7485 ctrl; pH 5 | .17 | 7.22 | .73 | 9.43 | .96 | .60 |
| S7485; T1208; D4161-48; | .14 | 7.07 | .74 | 0.85 | 3.87 | .56 |
| pH 5 | | | | | | |
| S7485; T1208; D4161-25; | .45 | 9.98 | .96 | 8.09 | 3.28 | .96 |
| pH 5 | | | | | | |
| S7485; T1208; D4161-10; | .07 | 6.91 | .83 | 2.50 | 2.45 | .53 |
| pH 5 | | | | | | |
| S7485; T1208; D4161-18; | .04 | 6.49 | .79 | 3.20 | 2.21 | .51 |
| pH 5 | | | | | | |
| S7485; T1208; D4161-47; | .31 | 8.16 | .77 | 2.42 | 1.04 | .60 |
| pH 5 | | | | | | |

TABLE 66
Unsaturated fatty acid profile in S7485 and representative derivative
transgenic lines transformed with pSZ5301 (LimdLPCAT2) at PLSC-2/
LPAAT1-1 genomic locus) DNA.

| Sample ID | 14:0 | 16:0 | 18:0 | 18:1 | 18:2 | 18:3a |
|---|---|---|---|---|---|---|
| S7485 ctrl; pH 5 | .15 | 7.14 | .72 | 9.62 | .94 | .58 |
| S7485 ctrl; pH 5 | .17 | 7.22 | .73 | 9.43 | .96 | .60 |
| S7485; T1208; D4162-36; | .21 | 6.64 | .76 | 6.44 | .55 | .59 |
| pH 5 | | | | | | |
| S7485; T1208; D4162-47; | .38 | 3.05 | .18 | 1.20 | .88 | .43 |
| pH 5 | | | | | | |
| S7485; T1208; D4162-38; | .51 | 0.48 | .53 | 4.94 | .34 | .59 |
| pH 5 | | | | | | |
| S7485; T1208; D4162-21; | .09 | 6.70 | .75 | 7.98 | .19 | .57 |
| pH 5 | | | | | | |
| S7485; T1208; D4162-5; pH 5 | .03 | 5.68 | .81 | 9.08 | .16 | .48 |

TABLE 67
Unsaturated fatty acid profile in S7485 and representative derivative
transgenic lines transformed with pSZ5307 (AtLPCAT1) at PLSC-2/
LPAAT1-2 genomic locus) DNA.

| Sample ID | 14:0 | 16:0 | 18:0 | 18:1 | 18:2 | 18:3 a |
|---|---|---|---|---|---|---|
| S7485 ctrl; pH 5 | .15 | 7.14 | .72 | 9.62 | .94 | .58 |
| S7485 ctrl; pH 5 | .17 | 7.22 | .73 | 9.43 | .96 | .60 |
| S7485; T1208; D4168-43; | .19 | 4.43 | .77 | 3.47 | 3.88 | .52 |
| pH 5 | | | | | | |
| S7485; T1208; D4168-18; | .44 | 7.39 | .18 | 1.73 | 2.93 | .65 |
| pH 5 | | | | | | |
| S7485; T1208; D4168-25; | .19 | 7.60 | .17 | 1.28 | 2.74 | .89 |
| pH 5 | | | | | | |
| S7485; T1208; D4168-16; | .14 | 3.48 | .00 | 4.53 | 2.64 | .92 |
| pH 5 | | | | | | |
| S7485; T1208; D4168-23; | .14 | 7.50 | .62 | 2.58 | 1.89 | .55 |
| pH 5 | | | | | | |

TABLE 68
Unsaturated fatty acid profile in S7485 and representative derivative
transgenic lines transformed with pSZ5308 (AtLPCAT2) at PLSC-2/
LPAAT1-2 genomic locus) DNA.

| Sample ID | 14:0 | 16:0 | 18:0 | 18:1 | 18:2 | 18:3a |
|---|---|---|---|---|---|---|
| S7485 ctrl; pH 5 | .15 | 7.14 | .72 | 9.62 | .94 | .58 |
| S7485 ctrl; pH 5 | .17 | 7.22 | .73 | 9.43 | .96 | .60 |
| S7485; T1208; D4169-26; | .47 | 9.39 | .33 | 8.33 | 5.31 | .51 |
| pH 5 | | | | | | |
| S7485; T1208; D4169-41; | .24 | 8.20 | .82 | 9.81 | 4.20 | .64 |
| pH 5 | | | | | | |
| S7485; T1208; D4169-19; | .28 | 9.52 | .98 | 9.26 | 2.89 | .86 |
| pH 5 | | | | | | |
| S7485; T1208; D4169-38; | .23 | 7.87 | .75 | 1.25 | 2.66 | .55 |
| pH 5 | | | | | | |
| S7485; T1208; D4169-37; | .19 | 7.52 | .79 | 1.59 | 2.62 | .56 |
| pH 5 | | | | | | |

TABLE 69
Unsaturated fatty acid profile in S7485 and representative derivative
transgenic lines transformed with pSZ5309 (BrLPCAT) at PLSC-2/
LPAAT1-2 genomic locus) DNA.

| Sample ID | 14:0 | 16:0 | 18:0 | 18:1 | 18:2 | 18:3 a |
|---|---|---|---|---|---|---|
| S7485; pH 5 | .15 | 7.16 | .72 | 9.63 | .91 | .56 |
| S7485; pH 5 | .18 | 7.24 | .74 | 9.45 | .94 | .57 |
| S7485; T1208; D4170-43; | .55 | 1.35 | .19 | 6.95 | 4.78 | .59 |
| pH 5 | | | | | | |
| S7485; T1208; D4170-46; | .14 | 7.43 | .76 | 1.94 | 2.52 | .58 |
| pH 5 | | | | | | |
| S7485; T1208; D4170-40; | .16 | 7.87 | .79 | 1.54 | 2.42 | .56 |
| pH 5 | | | | | | |
| S7485; T1208; D4170-42; | .07 | 8.06 | .74 | 1.69 | 2.30 | .54 |
| pH 5 | | | | | | |
| S7485; T1208; D4170-4; | .13 | 7.53 | .65 | 2.27 | 2.24 | .54 |
| pH 5 | | | | | | |

TABLE 70
Unsaturated fatty acid profile in S7485 and representative derivative
transgenic lines transformed with pSZ5309 (LimLPCAT2) at PLSC-2/
LPAAT1-2 genomic locus) DNA.

| Sample ID | 14:0 | 16:0 | 18:0 | 18:1 | 18:2 | 18:3 a |
|---|---|---|---|---|---|---|
| S7485 ctrl; pH 5 | .15 | 7.16 | .72 | 9.63 | .91 | .56 |
| S7485 ctrl; pH 5 | .18 | 7.24 | .74 | 9.45 | .94 | .57 |
| S7485; T1208; D4171-15; | .99 | 4.46 | .81 | 8.50 | .16 | .48 |
| pH 5 | | | | | | |
| S7485; T1208; D4171-30; | .14 | 5.91 | .81 | 7.62 | .30 | .55 |
| pH 5 | | | | | | |
| S7485; T1208; D4171-34; | .17 | 6.77 | .94 | 8.09 | .81 | .55 |
| pH 5 | | | | | | |
| S7485; T1208; D4171-43; | .01 | 5.75 | .88 | 9.47 | .78 | .51 |
| pH 5 | | | | | | |
| S7485; T1208; D4171-13; | .04 | 6.11 | .81 | 9.24 | .66 | .49 |
| pH 5 | | | | | | |

### Example 12

### Expression of LPCAT in a High-Erucic Transgenic Microalga

In this example we demonstrate the use of higher plant Lysophosphatidylcholine acyltransferase (LPCAT) genes to alter the content and composition of oils in transgenic algal strains for producing oils rich in linoleic and/or very long chain fatty acids (VLCFA).

The LPCAT genes from Example 11 herein were expressed in S7211. S7211was. Our results show that expression of heterologous LPCAT enzymes in S7211 results in more than 3 fold enhancement in linoleic (C18:2) and erucic (C22:1) acid content in individual lines over the parents.

Construct Used for the Expression of the A. thaliana Lysophosphatidylcholine Acyltransferase AtLPCAT) in Strain S7211 [pSZ5296]:

In this example, S7211, transformed with the construct pSZ5296, were generated which express Sacharomyces carlbergenesis MEL1 gene (allowing for their selection and growth on medium containing melibiose) and A. thaliana LPCAT gene targeted at endogenous PmLPAAT1-1 genomic region. Construct can be written as PLSC-2/LPAAT1-1 5′ flank::PmHXT1-ScarMEL1-CvNR:PmSAD2-2v2-AtLPCAT1-CvNR::PLSC-2/LPAAT1-1 3′ flank.

The sequence of the transforming DNA is provided below. Relevant restriction sites in the construct are indicated in lowercase, underlined bold, and are from 5′-3′ BspQI, KpnI, SpeI, SnaBI, EcoRI, SpeI, AflII, SacI, BspQI, respectively. BspQI sites delimit the 5′ and 3′ ends of the transforming DNA. Bold, lowercase sequences represent genomic DNA from S3150 that permit targeted integration at the PLSC-2/LPAAT1-1 locus via homologous recombination. Proceeding in the 5′ to 3′ direction, the endogenous P. moriformis Hexose Transporter 1 promoter driving the expression of the S. carlbergenesis MEL1 gene is indicated by lowercase, boxed text. The initiator ATG and terminator TGA for MEL1 are indicated by uppercase italics, while the coding region is indicated with lowercase italics. The Chlorella vulgaris nitrate reductase (NR) gene 3′ UTR is indicated by lowercase underlined text followed by PmSAD2-2v2. promoter of P. moriformis, indicated by boxed italicized text. The Initiator ATG and terminator TGA codons of the AtLPCAT1 are indicated by uppercase, bold italics, while the remainder of the gene is indicated by bold italics. The C. vulgaris nitrate reductase 3′ UTR is again indicated by lowercase underlined text followed by the P. moriformis PLSC-2/LPAAT1-1 genomic region indicated by bold, lowercase text. The final construct was sequenced to ensure correct reading frames and targeting sequences.

Nucleotide sequence of transforming DNA contained in plasmid pSZ5296:
(SEQ ID NO: 117)
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccctttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccacctg

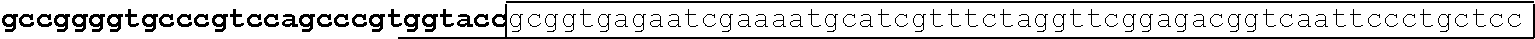

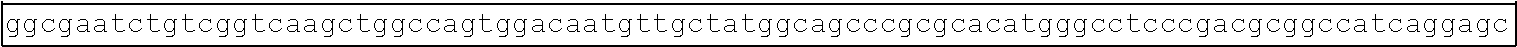

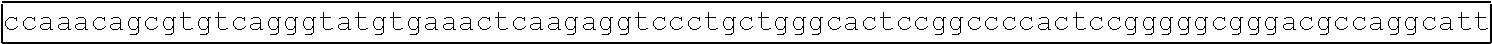

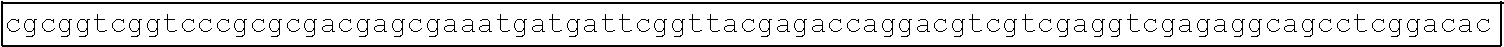

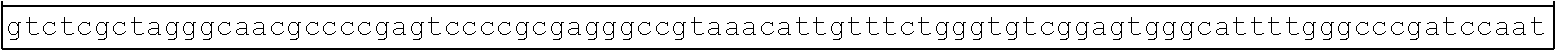

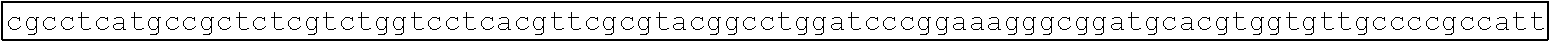

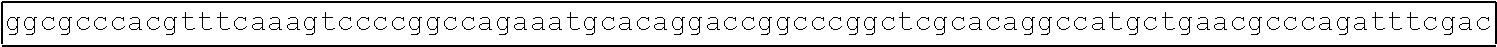

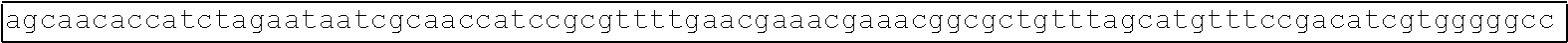

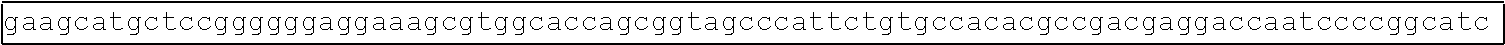

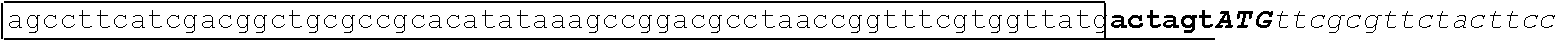

tgacggcctgcatctccctgaagggcgtgttcggcgtctccccctcctacaacggcctgggcctgacgccccagatgggctggga
caactggaacacgttcgcctgcgacgtctccgagcagctgctgctggacacggccgaccgcatctccgacctgggcctgaagga
catgggctacaagtacatcatcctggacgactgctggtcctccggccgcgactccgacggcttcctggtcgccgacgagcagaag
ttccccaacggcatgggccacgtcgccgaccacctgcacaacaactccttcctgttcggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgttcggccagaagatcggctccctgtcccccaacgcgatcctgaacacg

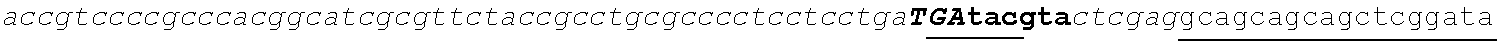

gtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtggg

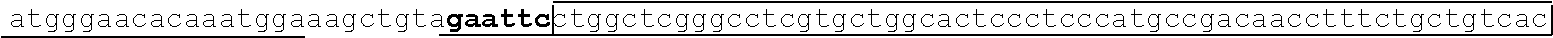

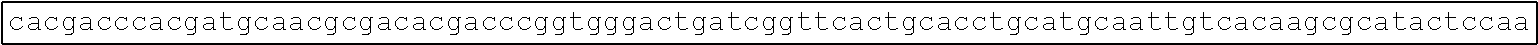

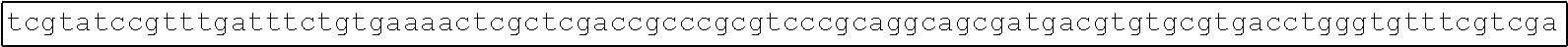

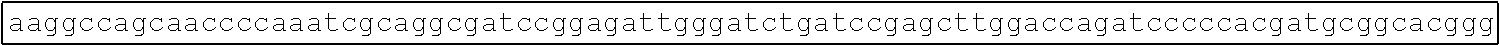

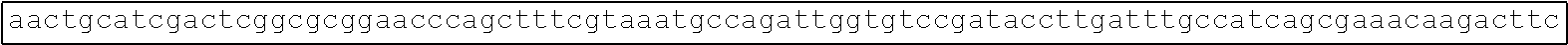

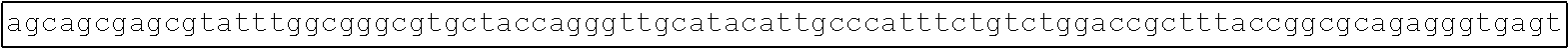

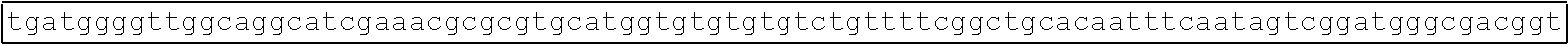

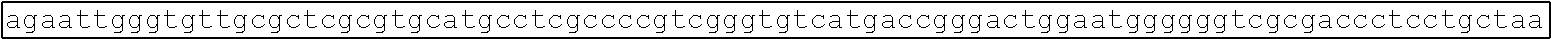

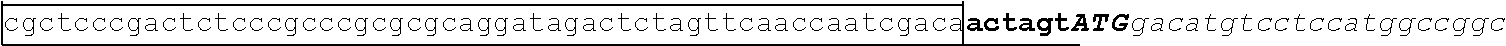

tccatcggcgtgtccgtggccgtgctgcgcttcctgctgtgcttcgtggccaccatccccgtgtccttcgcctgccgcatcgtgccctcc
cgcctgggcaagcacctgtacgccgccgcctccggcgccttcctgtcctacctgtccttcggcttctcctccaacctgcacttcctggt
gcccatgaccatcggctacgcctccatggccatctaccgccccaagtgcggcatcatcaccttcttcctgggcttcgcctacctgatc
ggctgccacgtgttctacatgtccggcgacgcctggaaggagggcggcatcgactccaccggcgccctgatggtgctgaccctga
aggtgatctcctgctccatgaactacaacgacggcatgctgaaggaggagggcctgcgcgaggcccagaagaagaaccgcct
gatccagatgccctccctgatcgagtacttcggctactgcctgtgctgcggctcccacttcgccggccccgtgtacgagatgaagga
ctacctggagtggaccgagggcaagggcatctgggacaccaccgagaagcgcaagaagccctccccctacggcgccaccatc
cgcgccatcctgcaggccgccatctgcatggccctgtacctgtacctggtgccccagtaccccctgacccgcttcaccgagcccgt
gtaccaggagtggggcttcctgcgcaagttctcctaccagtacatggccggcttcaccgcccgctggaagtactacttcatctggtc
catctccgaggcctccatcatcatctccggcctgggcttctccggctggaccgacgacgcctcccccaagcccaagtgggaccgc
gccaagaacgtggacatcctgggcgtggagctggccaagtccgccgtgcagatccccctggtgtggaacatccaggtgtccacc
tggctgcgccactacgtgtacgagcgcctggtgcagaacggcaagaaggccggcttcttccagctgctggccacccagaccgtgt
ccgccgtgtggcacggcctgtaccccggctacatgatgttcttcgtgcagtccgccctgatgatcgccggctcccgcgtgatctacc
gctggcagcaggccatctcccccaagatggccatgctgcgcaacatcatggtgttcatcaacttcctgtacaccgtgctggtgctga
actactccgccgtgggcttcatggtgctgtccctgcacgagaccctgaccgcctacggctccgtgtactacatcggcaccatcatcc

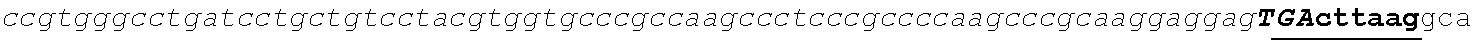

gcagcagctcggatagtatcgacacactctggacgctggtcgtgtgatggactgttgccgccacacttgctgccttgacctgtgaatatc
cctgccgcttttatcaaacagcctcagtgtgtttgatcttgtgtgtacgcgcttttgcgagttgctagctgcttgtgctatttgcgaataccac
ccccagcatccccttccctcgtttcatatcgcttgcatcccaaccgcaacttatctacgctgtcctgctatccctcagcgctgctcctgctc
ctgctcactgcccctcgcacagccttggtttgggctccgcctgtattctcctggtactgcaacctgtaaaccagcactgcaatgctgatgc
acgggaagtagtgggatgggaacacaaatggaaagcttaattaagagctccgtcctccactaccacagggtatggtcgtgtgggg
tcgagcgtgttgaagcgcagaaggggatgcgccgtcaagatcaggagctaaaaatggtgccagcgaggatccagcgctctc
actcttgctgccatcgctcccaccctttcccccaggggaccctgtggcccacgtgggagacgattccggccaagtggcacatctt
cctgatgctctgccacccccgccacaaagtgaccgtgatgaaggttaggacaagggtcgggacccgattctggatatgacctc
tgaggtgtgtttctcgcgcaagcgtcccccaattcgttacaccacatccctcacacctcgcccctgacactcgcagttgcccgt
gtacgtccccaatgaggaggaaaaggccgaccccaagctgtacgcccaaaacgtccgcaaagccatggtgcgtcgggaacc
gtcaaagtttgcttgcgggtgggcggggcggctctagcgaattggctcattggccctcaccgaggcagcacatcggacaccag
tcgccacccggctttgcatcttcgccccctttcttctcgcagatggaggtcgccgggaccaaggacacgacggcggtgttttgagg
acaagatgcgctacctgaactccctgaagagaaagtacggcaagcctgtgcctaagaaaattgagtgaacccccgtcgtcga
ccagaagagc

Constructs Used for the Expression of the AtLPCAT1 and AtLPCAT2, BrLPCAT, BjLPCAT1, BjLPCAT2, LimdLPCAT1 and LimdLPCAT2 Genes from Higher Plants in S7211:

In addition to the A. thaliana LPCAT1 targeted at PLSC-2/PmLPAAT1-1 locus (pSZ5296), A. thaliana LPCAT1 targeted at PLSC-2/LPAAT1-2 locus (pSZ5307), A. thaliana LPCAT2 targeted at PLSC-2/LPAAT1-1 locus (pSZ5297), A. thaliana LPCAT2 targeted at PLSC-2/LPAAT1-2 locus (pSZ5308), B. rapa LPCAT targeted at PLSC-2/PmLPAAT1-1 locus (pSZ5299), B. rapa LPCAT targeted at PLSC-2/PmLPAAT1-2 locus (pSZ5309), B. juncea LPCAT1 targeted at PLSC-2/PmLPAAT1-1 locus (pSZ5346), B. juncea LPCAT1 targeted at PLSC-2/PmLPAAT1-2 locus (pSZ5351), B. juncea LPCAT2 targeted at PLSC-2/PmLPAAT1-1 locus (pSZ5298), B. juncea LPCAT2 targeted at PLSC-2/PmLPAAT1-2 locus (pSZ5352), L. douglasii LPCAT1 targeted at PLSC-2/PmLPAAT1-1 locus (pSZ5300), L. douglasii LPCAT1 targeted at PLSC-2/PmLPAAT1-2 locus (pSZ5353), L. douglasii LPCAT2 targeted at PLSC-2/PmLPAAT1-1 locus (pSZ5301) and L. douglasii LPCAT2 targeted at PLSC-2/PmLPAAT1-2 locus (pSZ5310) have been constructed for expression in S7211. These constructs can be described as:

pSZ5307—PLSC-2/LPAAT1-2::PmHXT1-ScarMEL1-CvNR:PmSAD2-2v2-AtLPCAT1-CvNR::PLSC-2/LPAAT1-2
pSZ5297—PLSC-2/LPAAT1-1::PmHXT1-ScarMEL1-CvNR:PmSAD2-2v2-AtLPCAT2-CvNR::PLSC-2/LPAAT1-1
pSZ5308—PLSC-2/LPAAT1-2::PmHXT1-ScarMEL1-CvNR:PmSAD2-2v2-AtLPCAT2-CvNR::PLSC-2/LPAAT1-2
pSZ5299—PLSC-2/LPAAT1-1::PmHXT1-ScarMEL1-CvNR:PmSAD2-2v2-BrLPCAT-CvNR::PLSC-2/LPAAT1-1
pSZ5309—PLSC-2/LPAAT1-2::PmHXT1-ScarMEL1-CvNR:PmSAD2-2v2-BrLPCAT-CvNR::PLSC-2/LPAAT1-2
pSZ5346—PLSC-2/LPAAT1-1::PmHXT1-ScarMEL1-CvNR:PmSAD2-2v2-BjLPCAT1-CvNR::PLSC-2/LPAAT1-1
pSZ5351—PLSC-2/LPAAT1-2::PmHXT1-ScarMEL1-CvNR:PmSAD2-2v2-BjLPCAT1-CvNR::PLSC-2/LPAAT1-2
pSZ5298—PLSC-2/LPAAT1-1::PmHXT1-ScarMEL1-CvNR:PmSAD2-2v2-BjLPCAT2-CvNR::PLSC-2/LPAAT1-1
pSZ5352—PLSC-2/LPAAT1-2::PmHXT1-ScarMEL1-CvNR:PmSAD2-2v2-BjLPCAT2-CvNR::PLSC-2/LPAAT1-2
pSZ5300—PLSC-2/LPAAT1-1::PmHXT1-ScarMEL1-CvNR:PmSAD2-2v2-LimdLPCAT1-CvNR::PLSC-2/LPAAT1-1
pSZ5353—PLSC-2/LPAAT1-2::PmHXT1-ScarMEL1-CvNR:PmSAD2-2v2-LimdLPCAT1-CvNR::PLSC-2/LPAAT1-2
pSZ5301—PLSC-2/LPAAT1-1::PmHXT1-ScarMEL1-CvNR:PmSAD2-2v2-LimdLPCAT2-CvNR::PLSC-2/LPAAT1-1
pSZ5310—PLSC-2/LPAAT1-2::PmHXT1-ScarMEL1-CvNR:PmSAD2-2v2-LimdLPCAT2-CvNR::PLSC-2/LPAAT1-2

All these constructs have the same vector backbone; selectable marker, promoters, and 3′ utr as pSZ5296, differing only in either the genomic region used for construct targeting and/or the respective LPCAT gene. Relevant restriction sites in these constructs are also the same as in pSZ5296. The sequence of PLSC-2/LPAAT1-2 5′ flank, PLSC-2/LPAAT1-2 3′ flank and AtLPCAT1, AtLPCAT2, BrLPCAT, BjLPCAT1, BjLPCAT2, LimdLPCAT1 and LimdLPCAT2 genes respectively. Relevant restriction sites as bold text are shown 5′-3′ respectively are shown below.

Sequence of PLSC-2/LPAAT1-2 5′ flank in pSZ5307, pSZ5308, pSZ5309,
pSZ5310, pSZ5351, pSZ5352 and pSZ5353. PLSC-2/LPAAT1-2 5′ flank:
(SEQ ID NO: 118)
gctcttctgcttcggattccactacatcaagtgggtgaacctggcgggcgcggaggagggcccccgcccgggcggcattgtta
gcaaccactgcagctacctggacatcctgctgcacatgtccgactccttccccgcctttgtggcgcgccagtcgacggccaagc
tgccattatcggcatcatcaggtgcgtgaaagcgggggctgctgtggccgtggtgggcagggttgcgaaggggggcaggcg
taggcgtgcagtgtgagcggacattgatgccgtcgtttgccggtcaggagagctcgaaatcagagccagcctggtcatgggat
cacagagctcaccaccactcgtccacctcgcctgcgccttgcagccaaatcatgagctgcctctacgtgaaccgcgaccgctc
ggggcccaaccacgtgggcgtggccgatctggtgaagcagcgcatgcaggacgaggccgaggggaggaccccgcccgagt
accgaccgctgctcctcttccccgaggtgggctttcgaggcaccgtttgtgcttgaaactgtgggcacgcgtgccccgacgcgc
ctctggcgcctgcttcgcatccattcgcctctcaaccccgtctctcctttcctccatcgccagggcaccacctccaacggcgacta
cctgcttcccttcaagaccggcgccttcctggccggggtgcccgtccagcccgtggtacc
Sequence of PLSC-2/LPAAT1-2 3′ flank in pSZ5307, pSZ5308, pSZ5309,
pSZ5310, pSZ5351, pSZ5352 and pSZ5353. PLSC-2/LPAAT1-2 3′ flank:
(SEQ ID NO: 119)
gagctccgtcctccactaccacagggtatggtggtgtggggtcgagcgtgttgaagcgcggaaggggatgcgctgtcaagttt
tggagctgaaaatggtgcccgcgaggatccagcgcgccccactcacccttgctgccatcgctccccaccatttccccagggaa
ccctgtggcccacgtgggagacgattccggccaagtggcacatcttcctgatgctctgccacccccgccacaaagtgaccgtg
atgaaggtacgaacaagggtcgggccccgattctggatatcacgtctggggtgtglltctcgcgcacgcgtcccccgatgcgct
gcacagtctccctcacaccctcacccctaacgctcgcagttgcccgtgtacgtccccaatgaggaggaaaaggccgaccccaa
gctgtacgcccaaaatgttcgcaaagccatggtgcgtcgggaaccgttcaagtttgcttgcgggtgggcggggcggctctagc
gaattggcgcattggccctcaccgaggcagcacatcggacaccaatcgtcacccggcgagcaattccgccccctctgtcttctc
gcagatggaggtcgccgggaccaaggacacgacggcggtgtttgaggacaagatgcgctacctgaactccctgaagagaaa
gtacggcaagcctgtgcctaagaaaattgagtgaacccccgtcgtcgaccagaagagc
Nucleotide sequence of A. thaliana LPCAT 2 (AtLPCAT2) contained in
pSZ5297 and pSZ5308. AtLPCAT2:
(SEQ ID NO: 120)

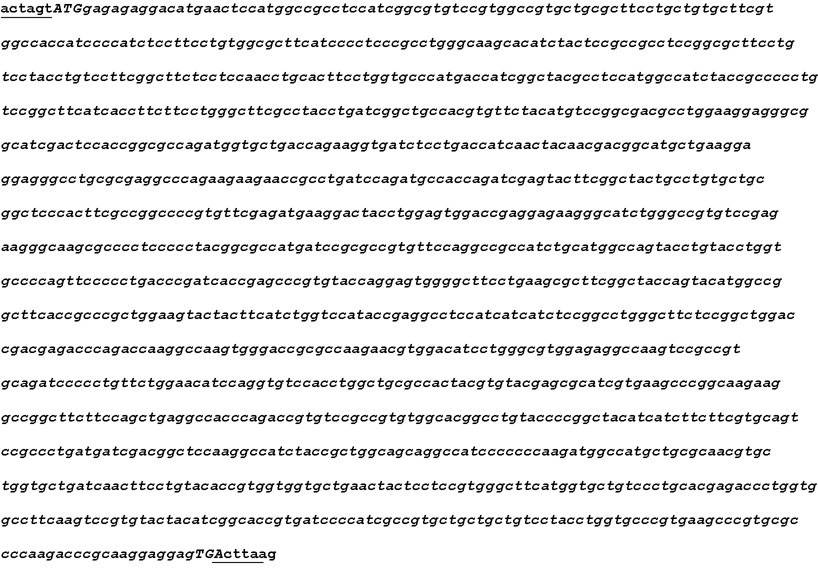

Nucleotide sequence of B. rapa LPCAT (BrLPCAT) contained in pSZ5299 and
pSZ5309. BrLPCAT:
(SEQ ID NO: 121)

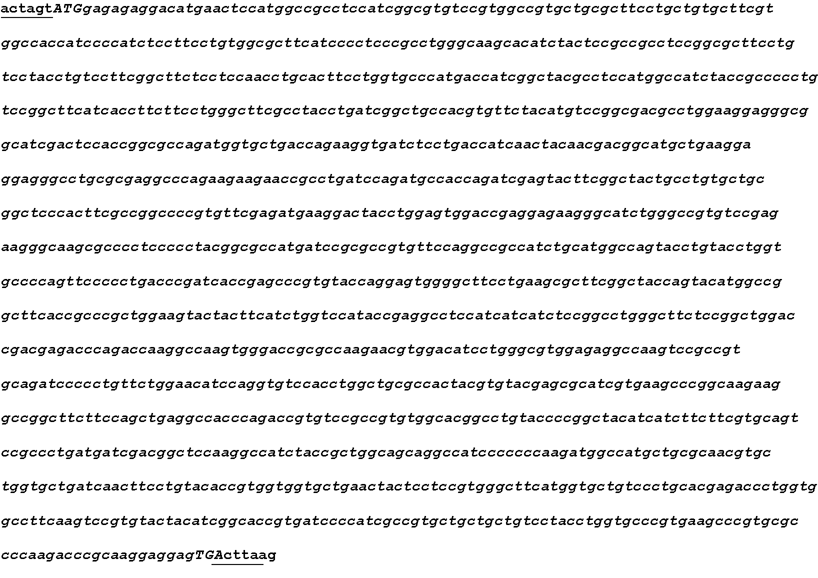

Nucleotide sequence of B. juncea LPCAT1 (BjLPCAT1) contained in pSZ5346
and pSZ5351. BjLPCAT1:
(SEQ ID NO: 122)

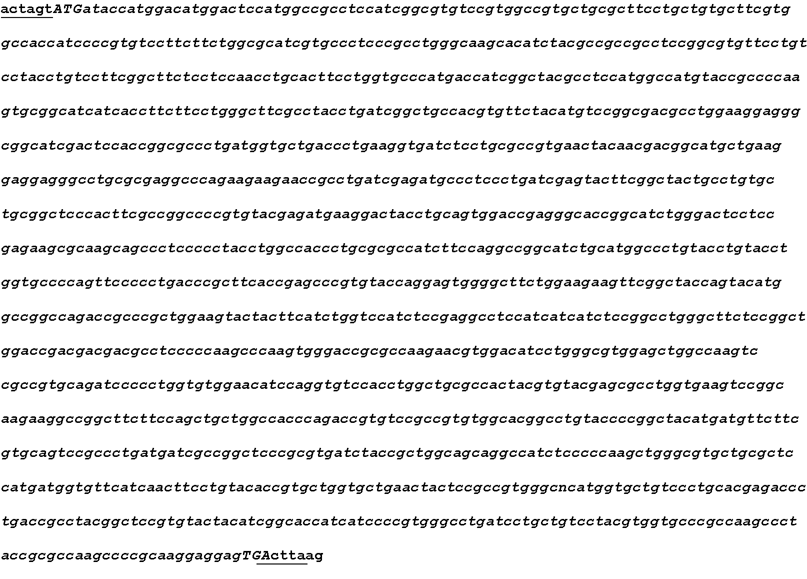

Nucleotide sequence of B. juncea LPCAT2 (BjLPCAT2) contained in pSZ5298
and pSZ5352. BjLPCAT2:
(SEQ ID NO: 123)

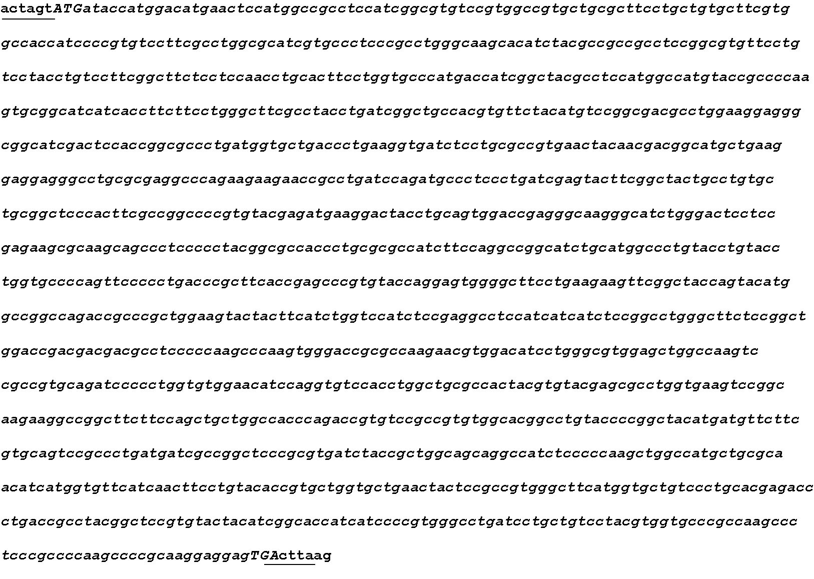

Nucleotide sequence of L. douglasii LPCAT1 (LimdLPCAT1) contained in
pSZ5300 and pSZ5353. LimdLPCAT1:
(SEQ ID NO: 124)

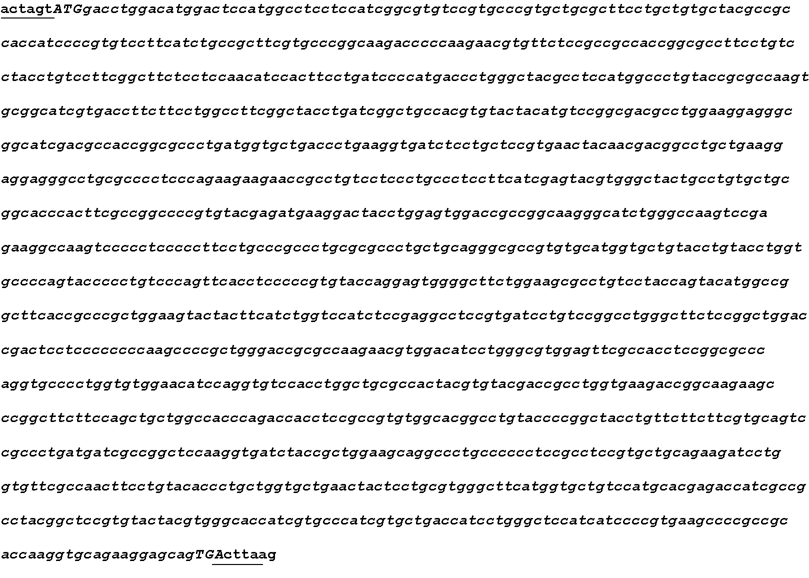

Nucleotide sequence of L. douglasii LPCAT2 (LimdLPCAT2) contained in
pSZ5301 and pSZ5310. LimdLPCAT2:
(SEQ ID NO: 125)

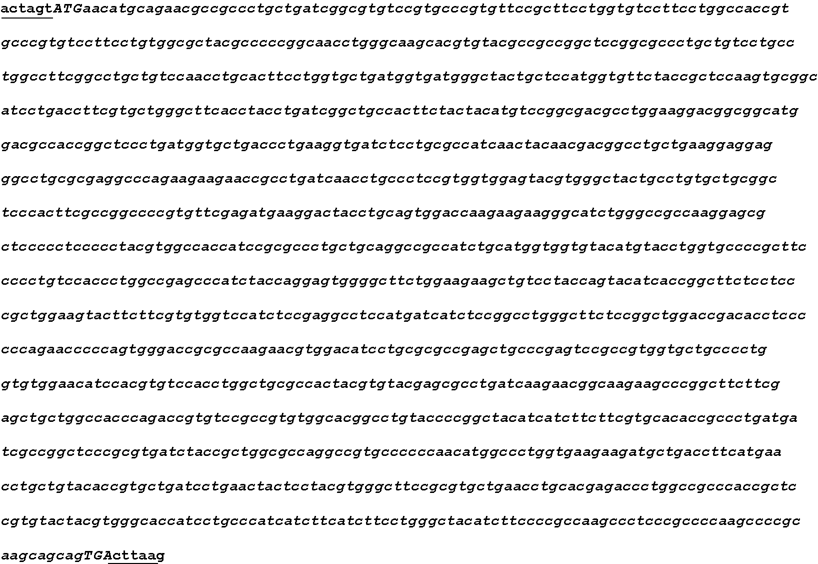

To determine their impact on fatty acid profiles, all the constructs described above were transformed independently into S7211. Primary transformants were clonally purified and grown under at pH7.0. S7211 expresses a FAE, from C. abyssinica under the control of pH regulated, AMT03 (Ammonium transporter 03) promoter. Thus both parental (S7211) and the resulting LPCAT transformed strains require growth at pH 7.0 to allow for maximal fatty acid elongase (FAE) gene expression. The resulting profiles from a set of representative clones arising from transformations with pSZ5296 (D4157), pSZ5307 (D4168), pSZ5297 (D4158), pSZ5308 (D4169), pSZ5299 (D4160), pSZ5309 (D4170), pSZ5346 (D4207), pSZ5351 (D4212), pSZ5298 (D4159), pSZ5352 (D4213), pSZ5300 (D4161), pSZ5353 (D4214), pSZ5301 (D4162) and pSZ5310 (D4171) into S7211 are shown in Tables 71-84 respectively.

All the transgenic lines expressing any of the above described LPCAT genes resulted in more than 2 fold increase in C18:2. The increase in C18:2 in S7211; T1172; D4157-14; pH7, expressing AtLPCAT1 at PLSC-2/LPAAT1-1 locus, was 2.54 fold (over parent S7211). These results demonstrate that heterologous LPCAT gene expression in our algal host enhances the conversion of C18:1-CoA into C18:1-PC. The PC associated C18:1 is subsequently acted upon by downstream enzymes like FAD2 and converted into C18:2. Concomitant with increase in C18:2 there was also significant and noticeable increase in C20:1 and C22:1. While the increase in C20:1 level was only 1.5-2 folds over the parent, the increase in C22:1 level was more than 3 fold in the majority of the genes tested at either LPAAT1-1 or LPAAT1-2 locus. In the case of S7211; T1174; D4171-11; pH7 the increase in C22:1 level was 5.3 fold (7.23%) over the parent (1.36%). Similarly in the case of S7211; T1173; D4162-10; pH7 the increase in C22:1 was 3.84 fold (5.23%) over the parent (1.36%). These are some of the highest C22:1 levels that we have obtained thus far in any algal base or transgenic strain. These results suggest that most likely the CrhFAE in S7211 uses C18:1-PC rather than C18:1-CoA as a substrate for elongation. In this scenario PmFAD2 and CrhFAE in S7211 would compete for the same substrate resulting in elevated C18:2 as well as VLCFA like C20:1 and C22:1. It would seem that PmFAD2-1 competes better for the substrate than CrhFAE.

Identification of LPCAT enzymes to increase conversion of C18:1 to C18:1-PC gives us a much better control over C18:1 phospholipid pool which can then be either directed towards making more polyunsaturated fatty acids or VLCFA by modulating the PmFAD2-1 activity.

TABLE 71
Unsaturated fatty acid profile in S3150, S7211 and representative
derivative transgenic lines transformed with pSZ5296
(AtLPCAT1 at PLSC-2/LPAAT1-1 genomic locus) DNA.

| Sample ID | 18:1 | 18:2 | 18:3a | um C20:1 | 22:1 |
|---|---|---|---|---|---|
| S7211; T1172; D4157-14; pH 7 | 3.75 | 4.59 | .72 | .30 | .17 |
| S7211; T1172; D4157-5; pH 7 | 2.42 | 1.22 | .47 | .99 | .04 |
| S7211; T1172; D4157-15; pH 7 | 3.70 | 0.99 | .38 | .94 | .88 |
| S7211; T1172; D4157-20; pH 7 | 2.46 | 1.19 | .41 | .87 | .78 |
| S7211; T1172; D4157-8; pH 7 | 2.77 | 0.88 | .41 | .86 | .72 |
| S7211A; pH 7 | 8.10 | .65 | .78 | .03 | .34 |
| S7211B; pH 7 | 8.11 | .64 | .77 | .01 | .33 |
| S3150; pH 7 | 7.99 | .62 | .56 | .19 | .00 |
| S3150; pH 5 | 7.70 | .08 | .54 | .11 | .00 |

TABLE 72
Unsaturated fatty acid profile in S3150, S7211 and representative
derivative transgenic lines transformed with pSZ5307
(AtLPCAT1 at PLSC-2/LPAAT1-2 genomic locus) DNA.

| Sample ID | C18:1 | C18:2 | C18:3a | Sum C20:1 | C22:1 |
|---|---|---|---|---|---|
| S7211; T1173; | 31.13 | 21.20 | 1.73 | 4.96 | 4.44 |
| D4168-12; pH 7 | | | | | |
| S7211; T1173; | 33.12 | 20.26 | 1.52 | 4.90 | 4.08 |
| D4168-7; pH 7 | | | | | |
| S7211; T1173; | 32.86 | 20.82 | 1.60 | 4.63 | 3.79 |
| D4168-15; pH 7 | | | | | |
| S7211; T1173; | 32.34 | 21.12 | 1.67 | 4.77 | 3.67 |
| D4168-1; pH 7 | | | | | |
| S7211; T1173; | 32.86 | 20.83 | 1.54 | 4.75 | 3.67 |
| D4168-3; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 58 | 6.62 | 0.56 | 0.19 | 0.0 |
| S3150; pH 5 | 57.7 | 7.08 | 0.54 | 0.11 | 0.0 |

TABLE 73
Unsaturated fatty acid profile in S3150, S7211 and representative
derivative transgenic lines transformed with pSZ5297
(AtLPCAT2 at PLSC-2/LPAAT1-1 genomic locus) DNA.

| Sample ID | C18:1 | C18:2 | C18:3a | Sum C20:1 | C22:1 |
|---|---|---|---|---|---|
| S7211; T1172; | 27.68 | 22.42 | 1.72 | 4.60 | 5.56 |
| D4158-4; pH 7 | | | | | |
| S7211; T1172; | 31.76 | 21.24 | 1.38 | 4.75 | 4.14 |
| D4158-18; pH 7 | | | | | |
| S7211; T1172; | 22.59 | 23.56 | 1.63 | 4.38 | 4.09 |
| D4158-5; pH 7 | | | | | |
| S7211; T1172; | 21.74 | 23.81 | 1.75 | 4.35 | 4.04 |
| D4158-1; pH 7 | | | | | |
| S7211; T1172; | 31.29 | 21.82 | 1.45 | 4.90 | 3.95 |
| D4158-25; pH 7 | | | | | |
| S7211A; pH 7 | 48.23 | 9.69 | 0.75 | 4.02 | 1.34 |
| S7211B; pH 7 | 48.24 | 9.65 | 0.75 | 4.01 | 1.33 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 74
Unsaturated fatty acid profile in S3150, S7211 and representative
derivative transgenic lines transformed with pSZ5308
(AtLPCAT2 at PLSC-2/LPAAT1-2 genomic locus) DNA.

| Sample ID | C18:1 | C18:2 | C18:3a | Sum C20:1 | C22:1 |
|---|---|---|---|---|---|
| S7211; T1174; | 31.32 | 20.66 | 1.79 | 4.95 | 3.51 |
| D4169-7; pH 7 | | | | | |
| S7211; T1174; | 32.20 | 20.47 | 1.78 | 4.83 | 3.29 |
| D4169-1; pH 7 | | | | | |
| S7211; T1174; | 39.33 | 17.63 | 0.88 | 4.29 | 1.79 |
| D4169-2; pH 7 | | | | | |
| S7211; T1174; | 39.99 | 17.17 | 0.83 | 3.91 | 1.76 |
| D4169-3; pH 7 | | | | | |
| S7211; T1174; | 37.46 | 17.54 | 0.97 | 3.99 | 1.73 |
| D4169-8; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 75
Unsaturated fatty acid profile in S3150, S7211 and representative
derivative transgenic lines transformed with pSZ5299
(BrLPCAT at PLSC-2/LPAAT1-1 genomic locus) DNA.

| Sample ID | C:18:1 | C18:2 | C18:3a | Sum C20:1 | C22:1 |
|---|---|---|---|---|---|
| S7211; T1172; | 42.75 | 15.97 | 1.87 | 6.42 | 4.14 |
| D4160-13; pH 7 | | | | | |
| S7211; T1172; | 31.80 | 21.32 | 1.42 | 4.66 | 3.58 |
| D4160-10; pH 7 | | | | | |
| S7211; T1172; | 33.68 | 21.02 | 1.36 | 4.52 | 3.17 |
| D4160-5; pH 7 | | | | | |
| S7211; T1172; | 32.50 | 21.86 | 1.37 | 4.34 | 3.03 |
| D4160-3; pH 7 | | | | | |
| S7211; T1172; | 31.07 | 22.48 | 1.68 | 3.78 | 3.02 |
| D4160-12; pH 7 | | | | | |
| S7211A; pH 7 | 48.10 | 9.65 | 0.78 | 4.03 | 1.34 |
| S7211B; pH 7 | 48.11 | 9.64 | 0.77 | 4.01 | 1.33 |
| S3150; pH 7 | 58.00 | 6.62 | 0.56 | 0.19 | 0.00 |
| S3150; pH 5 | 57.7 | 7.08 | 0.54 | 0.11 | 0.00 |

TABLE 76
Unsaturated fatty acid profile in S3150, S7211 and representative
derivative transgenic lines transformed with pSZ5309
(BrLPCAT at PLSC-2/LPAAT1-2 genomic locus) DNA.

| Sample ID | C18:1 | C18:2 | C18:3a | Sum C20:1 | C22:1 |
|---|---|---|---|---|---|
| S7211; T1174; | 31.46 | 20.98 | 1.69 | 4.53 | 3.33 |
| D4170-9; pH 7 | | | | | |
| S7211; T1174; | 29.68 | 22.07 | 1.77 | 4.29 | 3.12 |
| D4170-7; pH 7 | | | | | |
| S7211; T1174; | 38.98 | 17.16 | 0.92 | 3.76 | 1.63 |
| D4170-6; pH 7 | | | | | |
| S7211; T1174; | 34.80 | 18.50 | 0.95 | 3.60 | 1.51 |
| D4170-3; pH 7 | | | | | |
| S7211; T1174; | 40.55 | 16.64 | 0.91 | 3.68 | 1.50 |
| D4170-5; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 77
Unsaturated fatty acid profile in S3150, S7211 and representative
derivative transgenic lines transformed with pSZ5346
(BjLPCAT1 at PLSC-2/LPAAT1-1 genomic locus) DNA.

| Sample ID | C18:1 | C18:2 | C18:3a | C20:1 | C22:1 |
|---|---|---|---|---|---|
| S7211; T1181; D4207-4; | 29.69 | 21.89 | 1.79 | 5.04 | 4.50 |
| pH 7 | | | | | |
| S7211; T1181; D4207-6; | 32.55 | 20.69 | 1.56 | 4.71 | 3.68 |
| pH 7 | | | | | |
| S7211; T1181; | 36.16 | 17.75 | 1.51 | 3.89 | 1.83 |
| D4207-12; pH 7 | | | | | |
| S7211; T1181; D4207-2; | 40.69 | 16.61 | 0.94 | 3.74 | 1.58 |
| pH 7 | | | | | |
| S7211; T1181; | 38.53 | 17.69 | 1.15 | 3.66 | 1.47 |
| D4207-21; pH 7 | | | | | |
| S7211; pH 7 | 47.81 | 10.21 | 0.88 | 4.27 | 1.54 |
| S7211; pH 7 | 47.96 | 10.11 | 0.90 | 4.28 | 1.55 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 78
Unsaturated fatty acid profile in S3150, S7211 and representative
derivative transgenic lines transformed with pSZ5351
(BjLPCAT1 at PLSC-2/LPAAT1-2 genomic locus) DNA.

| Sample ID | C18:1 | C18:2 | C18:3 a | Sum C20:1 | C22:1 |
|---|---|---|---|---|---|
| S7211; T1181; | 32.19 | 20.59 | 1.66 | 4.75 | 3.13 |
| D4212-19; pH 7 | | | | | |
| S7211; T1181; | 38.65 | 19.57 | 1.73 | 4.41 | 2.70 |
| D4212-16; pH 7 | | | | | |
| S7211; T1181; | 37.23 | 17.56 | 1.12 | 4.14 | 2.59 |
| D4212-4; pH 7 | | | | | |
| S7211; T1181; | 40.99 | 17.16 | 0.99 | 3.88 | 1.74 |
| D4212-7; pH 7 | | | | | |
| S7211; T1181; | 40.35 | 17.23 | 1.00 | 3.82 | 1.74 |
| D4212-10; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 79
Unsaturated fatty acid profile in S3150, S7211 and representative
derivative transgenic lines transformed with pSZ5298
(BjLPCAT2 at PLSC-2/LPAAT1-1 genomic locus) DNA.

| Sample ID | C18:1 | C18:2 | C18:3a | Sum C20:1 | C22:1 |
|---|---|---|---|---|---|
| S7211; T1172; | 31.41 | 22.58 | 1.29 | 4.65 | 3.55 |
| D4159-1; pH 7 | | | | | |
| S7211; T1172; | 34.25 | 19.66 | 1.34 | 4.63 | 3.29 |
| D4159-4; pH 7 | | | | | |
| S7211; T1172; | 33.63 | 21.08 | 1.39 | 4.51 | 3.00 |
| D4159-2; pH 7 | | | | | |
| S7211; T1172; | 32.92 | 21.65 | 1.32 | 4.29 | 2.78 |
| D4159-5; pH 7 | | | | | |
| S7211; T1172; | 40.83 | 16.13 | 0.80 | 4.24 | 1.75 |
| D4159-3; pH 7 | | | | | |
| S7211A; pH 7 | 48.10 | 9.65 | 0.78 | 4.03 | 1.34 |
| S7211B; pH 7 | 48.11 | 9.64 | 0.77 | 4.01 | 1.33 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 80
Unsaturated fatty acid profile in S3150, S7211 and representative
derivative transgenic lines transformed with pSZ5352
(BjLPCAT2 at PLSC-2/LPAAT1-2 genomic locus) DNA.

| Sample ID | C18:1 | C18:2 | C18:3a | Sum C20:1 | C22:1 |
|---|---|---|---|---|---|
| S7211; T1181; | 42.85 | 11.60 | 1.14 | 4.56 | 2.43 |
| D4213-8; pH 7 | | | | | |
| S7211; T1181; | 37.35 | 18.74 | 1.38 | 4.04 | 2.23 |
| D4213-10; pH 7 | | | | | |
| S7211; T1181; | 39.13 | 17.39 | 1.06 | 3.84 | 2.00 |
| D4213-2; pH 7 | | | | | |
| S7211; T1181; | 40.16 | 17.18 | 1.02 | 3.83 | 1.77 |
| D4213-4; pH 7 | | | | | |
| S7211; T1181; | 39.01 | 17.52 | 1.22 | 3.86 | 1.69 |
| D4213-9; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 81
Unsaturated fatty acid profile in S3150, S7211 and
representative derivative transgenic lines transformed
with pSZ5300 (LimdLPCAT1 at PLSC-2/LPAAT1-1 genomic
locus) DNA.

| Sample ID | C18:1 | C18:2 | C18:3a | SumC20:1 | C22:1 |
|---|---|---|---|---|---|
| S7211; T1173; | 38.70 | 13.22 | 1.42 | 5.92 | 4.02 |
| D4161-1; pH 7 | | | | | |
| S7211; T1173; | 34.45 | 19.36 | 1.46 | 5.14 | 3.94 |
| D4161-10; pH 7 | | | | | |
| S7211; T1173; | 39.15 | 12.89 | 1.43 | 5.80 | 3.90 |
| D4161-2; pH 7 | | | | | |
| S7211; T1173; | 33.94 | 19.19 | 1.49 | 5.00 | 3.74 |
| D4161-9; pH 7 | | | | | |
| S7211; T1173; | 34.36 | 19.61 | 1.48 | 5.01 | 3.70 |
| D4161-5; pH 7 | | | | | |
| S7211A; pH 7 | 48.23 | 9.69 | 0.75 | 4.02 | 1.34 |
| S7211B; pH 7 | 48.24 | 9.65 | 0.75 | 4.01 | 1.33 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 82
Unsaturated fatty acid profile in S3150, S7211 and
representative derivative transgenic lines transformed
with pSZ5353 (LimdLPCAT1 at PLSC-2/LPAAT1-2 genomic
locus) DNA.

| | | | | Sum | |
|---|---|---|---|---|---|
| Sample ID | C18:1 | C18:2 | C18:3a | C20:1 | C22:1 |
| S7211; T1181; | 34.11 | 19.55 | 1.70 | 5.13 | 3.96 |
| D4214-10; pH 7 | | | | | |
| S7211; T1181; | 34.31 | 19.37 | 1.82 | 5.02 | 3.76 |
| D4214-24; pH 7 | | | | | |
| S7211; T1181; | 35.81 | 19.18 | 1.71 | 4.77 | 3.10 |
| D4214-6; pH 7 | | | | | |
| S7211; T1181; | 39.90 | 17.88 | 1.02 | 4.20 | 1.79 |
| D4214-15; pH 7 | | | | | |
| S7211; T1181; | 42.15 | 16.56 | 0.93 | 4.04 | 1.72 |
| D4214-9; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 83
Unsaturated fatty acid profile in S3150, S7211 and
representative derivative transgenic lines transformed
with pSZ5301 (LimdLPCAT2 at PLSC-2/LPAAT1-1 genomic
locus) DNA.

| | | | | Sum | |
|---|---|---|---|---|---|
| Sample ID | C18:1 | C18:2 | C18:3 a | C20:1 | C22:1 |
| S7211; T1173; | 38.40 | 17.61 | 1.86 | 7.29 | 5.28 |
| D4162-10; pH 7 | | | | | |
| S7211; T1173; | 37.73 | 13.94 | 1.27 | 6.06 | 4.41 |
| D4162-1; pH 7 | | | | | |
| S7211; T1173; | 37.27 | 14.92 | 1.45 | 6.33 | 4.34 |
| D4162-11; pH 7 | | | | | |
| S7211; T1173; | 36.23 | 15.03 | 1.55 | 6.23 | 4.16 |
| D4162-2; pH 7 | | | | | |
| S7211; T1173; | 37.90 | 14.29 | 1.41 | 6.08 | 4.16 |
| D4162-9; pH 7 | | | | | |
| S7211A; pH 7 | 48.23 | 9.69 | 0.75 | 4.02 | 1.34 |
| S7211B; pH 7 | 48.24 | 9.65 | 0.75 | 4.01 | 1.33 |
| S3150; pH 7 | 58.00 | 6.62 | 0.56 | 0.19 | 0.00 |
| S3150; pH 5 | 57.70 | 7.08 | 0.54 | 0.11 | 0.00 |

TABLE 84
Unsaturated fatty acid profile in S3150, S7211 and
representative derivative transgenic lines transformed
with pSZ5310 (LimdLPCAT2 at PLSC-2/LPAAT1-2 genomic
locus) DNA.

| | | | | Sum | |
|---|---|---|---|---|---|
| Sample ID | C18:1 | C18:2 | C18:3a | C20:1 | C22:1 |
| S7211; T1174; | 26.00 | 17.76 | 2.44 | 6.63 | 7.23 |
| D4171-11; pH 7 | | | | | |
| S7211; T1174; | 32.30 | 19.30 | 0.97 | 7.56 | 5.37 |
| D4171-3; pH 7 | | | | | |
| S7211; T1174; | 36.47 | 14.36 | 1.30 | 5.75 | 3.86 |
| D4171-9; pH 7 | | | | | |
| S7211; T1174; | 37.07 | 15.14 | 1.49 | 5.86 | 3.75 |
| D4171-12; pH 7 | | | | | |
| S7211; T1174; | 39.18 | 13.71 | 1.54 | 5.68 | 3.41 |
| D4171-2; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 58.00 | 6.62 | 0.56 | 0.19 | 0.00 |
| S3150; pH 5 | 57.70 | 7.08 | 0.54 | 0.11 | 0.00 |

### Example 13

### Expression of Arabidopsis thaliana PDCT in High-Erucic and High-Oleic Transgenic Microalgae

In this example we demonstrate the use of Arabidopsis thaliana Phosphatidylcholine diacylglycerol cholinephosphotransferase (AtPDCT) gene to alter the content and composition of oils in transgenic algal strains for producing oils rich in linoleic and/or very long chain fatty acids (VLCFA).

Fatty acids produced in the plastids are not always immediately available for TAG biosynthesis. Diacylglycerol (DAG) represents an important branch point between non-polar and membrane lipid biosynthesis. DAGs may be converted to PC by CDP-choline:1,2-sn-diacylglycerol cholinephosphotransferase (DAG-CPT), and acyl residues are then further desaturated by fatty acid desaturases. There are at least two possible routes whereby acyl residues from PC are incorporated into TAG. First, the DAG moiety of PC can be liberated (by hydrolysis) by reversible action of DAG-CPT, thus becoming available for TAG assembly by DGAT. The second route involves an enzyme known as phosphatidylcholine:1,2-sn-diacylglycerol choline phosphotransferase (PDCT). Like DAG-CPT, the PDCT mediates a symmetrical inter-conversion between phosphatidylcholine (PC) and diacylglycerol (DAG), thus enriching PC-modified fatty acids—C18:2 and C18:3—in the DAG pool prior to forming TAG.

AtPDCT has been reported as a major pathway for inter-conversion between PC and DAG pools while DAG-CPT plays a minor role. In light of this information we decided to express AtPDCT in our algal host. We express AtPDCT in high erucic strain S7211. We also expressed the AtPDCT in classically mutagenized high oleic base strain S8028 which produces significantly more C18:1 (68%) than our base strain S3150 (57%) but does not produce erucic acid. S8028 is a strain made according to the methods disclosed in co-owned application No. 61/779,708 filed on 13 Mar. 2013. Specifically, S8028 is a cerulenin resistant isolate of Strain K with low C16:0 titer and high C18:1 titer made according to Example 14 of 61/779,708.

The sequence of AtPDCT was codon optimized for expression in our P. moriformis and transformed into S7211 and S8028. Our results show that expression of AtPDCT in both erucic strain S7211 and high oleic base strain S8028 results in more than 3 fold enhancement in linoleic (C18:2) in individual lines. Additionally in S7211 there is a noticeable increase in erucic (C22:1) acid content in individual lines over the parents.

Construct Used for the Expression of the A. thaliana Phosphatidylcholine Diacylglycerol Cholinephosphotransferase (AtPDCT) in S7211 and S8028 [pSZ5344]:

Construct pSZ5344 expresses Sacharomyces carlbergenesis MEL1 gene (allowing for their selection and growth on medium containing melibiose) and A. thaliana LPCAT gene targeted at endogenous PmLPAAT1-1 genomic region. Construct pSZ5344 can be written as PLSC-2/LPAAT1-1 5′ flank::PmHXT1-ScarMEL1-CvNR:PmSAD2-2v2-AtLPDCT-CvNR::PLSC-2/LPAAT1-1 3′ flank.

The sequence of the transforming DNA is provided in below. Relevant restriction sites in the construct are indicated in lowercase, underlined bold, and are from 5′-3′ BspQI, KpnI, SpeI, SnaBI, EcoRI, SpeI, AflII, SacI, BspQI, respectively. BspQI sites delimit the 5′ and 3′ ends of the transforming DNA. Bold, lowercase sequences represent genomic DNA from S3150 that permit targeted integration at the PLSC-2/LPAAT1-1 locus via homologous recombination. Proceeding in the 5′ to 3′ direction, the endogenous P. moriformis Hexose Transporter 1 promoter driving the expression of the S. carlbergenesis MEL1 gene (encoding an alpha galactosidase enzyme activity required for catabolic conversion of Meliobise to glucose and galactose, thereby permitting the transformed strain to grow on melibiose) is indicated by lowercase, boxed text. The initiator ATG and terminator TGA for MEL1 are indicated by uppercase italics, while the coding region is indicated with lowercase italics. The Chlorella vulgaris nitrate reductase (NR) gene 3′ UTR is indicated by lowercase underlined text followed by a PMSAD2-2 promoter of P. moriformis, indicated by boxed italicized text. The Initiator ATG and terminator TGA codons of the AtPDCT are indicated by uppercase, bold italics, while the remainder of the gene is indicated by bold italics. The C. vulgaris nitrate reductase 3′ UTR is again indicated by lowercase underlined text followed by the S3150 PLSC-2/LPAAT1-1 genomic region indicated by bold, lowercase text. The final construct was sequenced to ensure correct reading frames and targeting sequences.

Nucleotide sequence of transforming DNA contained in plasmid pSZ5344:
(SEQ ID NO: 126)
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccctttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg

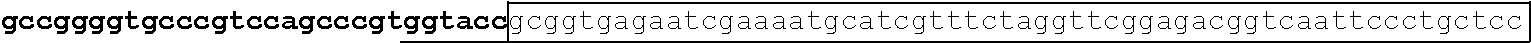

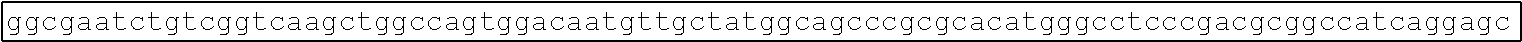

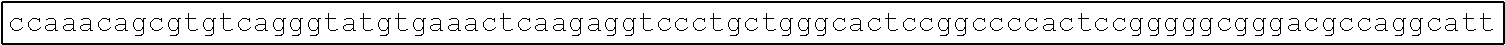

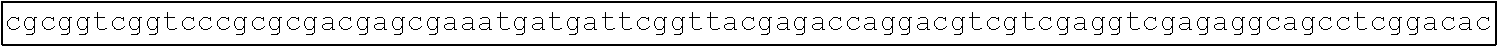

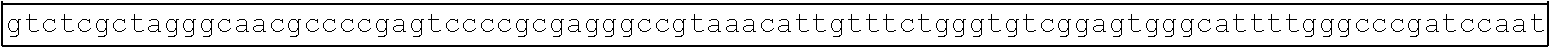

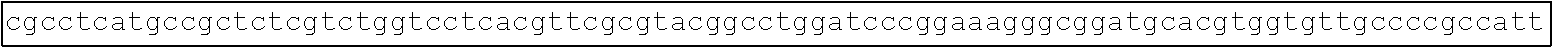

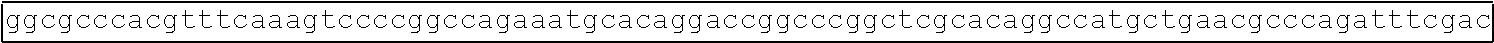

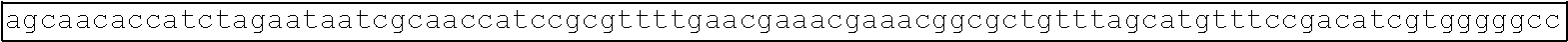

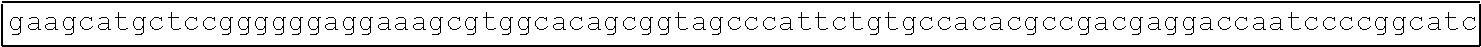

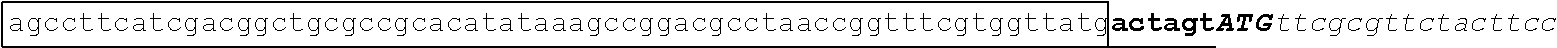

tgacggcctgcatctccctgaagggcgtgttcggcgtctccccctcctacaacggcctgggcctgacgccccagatgggctggga
caactggaacacgttcgcctgcgacgtctccgagcagctgctggacacggccgaccgcatctccgacctgggcctgaagga
catgggctacaagtacatcatcctggacgactgctggtcctccggccgcgactccgacggcttcctggtcgccgacgagcagaag
ttccccaacggcatgggccacgtcgccgaccacctgcacaacaactccttcctgttcggcatgtactcctccgcgggcgagtacac
gtgcgccggctaccccggctccctgggccgcgaggaggaggacgcccagttatcgcgaacaaccgcgtggactacctgaagt
acgacaactgctacaacaagggccagttcggcacgcccgagatctcctaccaccgctacaaggccatgtccgacgccagaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgttcggccagaagatcggctccctgtcccccaacgcgatcctgaacacg

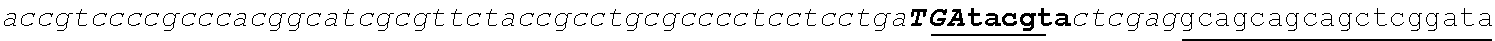

gtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtggg

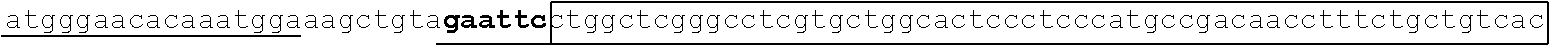

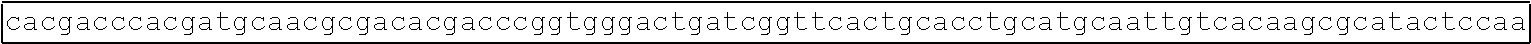

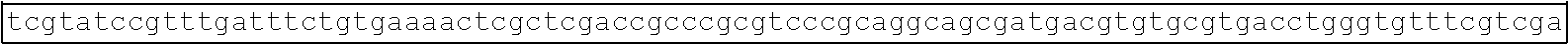

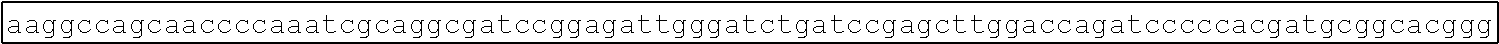

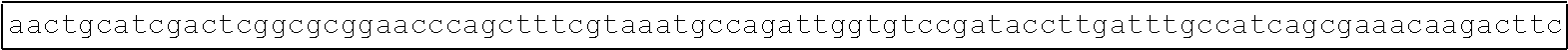

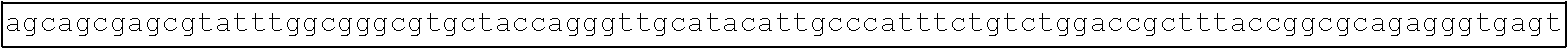

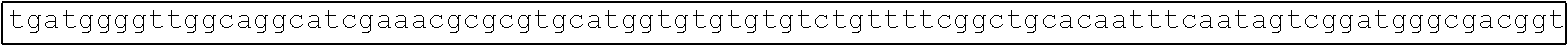

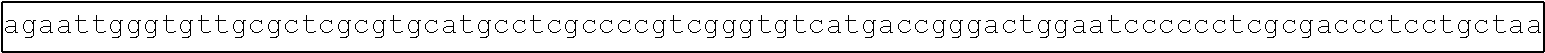

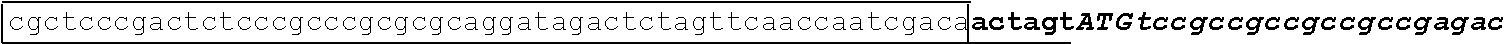

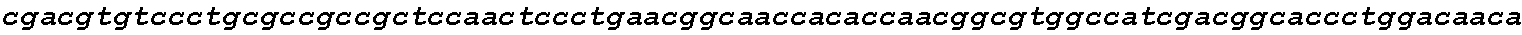

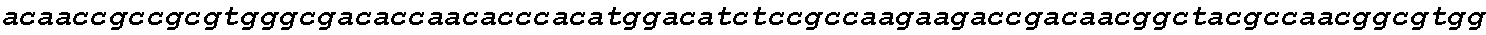

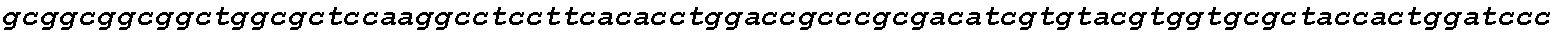

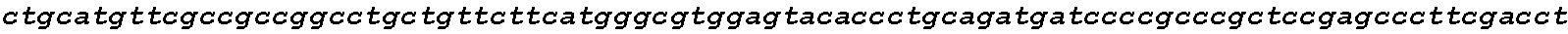

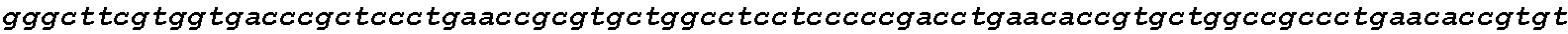

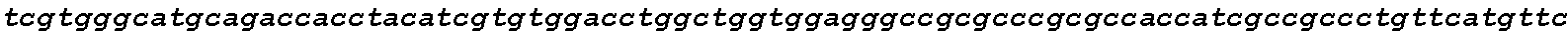

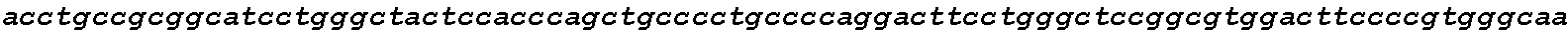

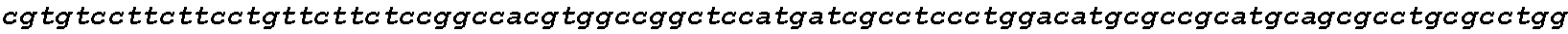

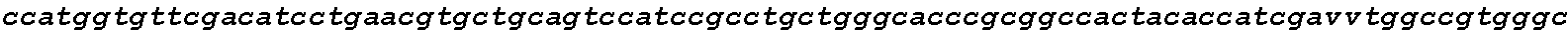

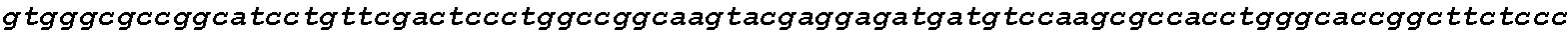

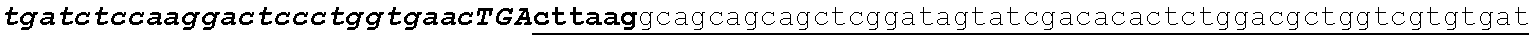

ggactgttgccgccacacttgctgccttgacctgtgaatatccctgccgcttttatcaaacagcctcagtgtgtttgatcttgtgtgtacgcg
cttttgcgagttgctagctgcttgtgctatttgcgaataccacccccagcatccccttccctcgtttcatatcgcttgcatcccaaccgcaac
ttatctacgctgtcctgctatccctcagcgctgctcctgctcctgctcactgcccctcgcacagccttggtttgggctccgcctgtattctcc
tggtactgcaacctgtaaaccagcactgcaatgctgatgcacgggaagtagtgggatgggaacacaaatggaaagcttaattaagag
ctccgtcctccactaccacagggtatggtcgtgtggggtcgagcgtgttgaagcgcagaaggggatgcgccgtcaagatcag
gagctaaaaatggtgccagcgaggatccagcgctctcactcttgctgccatcgctcccacccttttccccaggggaccctgtgg
cccacgtgggagacgattccggccaagtggcacatcttcctgatgctctgccacccccgccacaaagtgaccgtgatgaaggt
taggacaagggtcgggacccgattctggatatgacctctgaggtgtgtttctcgcgcaagcgtcccccaattcgttacaccaca
tccctcacaccctcgcccctgacactcgcagttgcccgtgtacgtccccaatgaggaggaaaaggccgaccccaagctgtacg
cccaaaacgtccgcaaagccatggtgcgtcgggaaccgtcaaagtttgcttgcgggtgggcggggcggctctagcgaattgg
ctcattggccctcaccgaggcagcacatcggacaccagtcgccacccggcttgcatcttcgcccctttcttctcgcagatggag
gtcgccgggaccaaggacacgacggcggtgtttgaggacaagatgcgctacctgaactccctgaagagaaagtacggcaag
cctgtgcctaagaaaattgagtgaacccccgtcgtcgaccagaagagc

Construct Used for the Expression of the AtPDCT at PLSC-2/PmLPAAT1-2 Locus in S7211 and S8028:

In addition to the A. thaliana PDCT targeted at PLSC-2/PmLPAAT1-1 locus (pSZ5344), A. thaliana PDCT targeted at PLSC-2/LPAAT1-2 locus (pSZ5349), was constructed for expression in both S7211 and S8028. The construct can be described as:

pSZ5349-PLSC-2/LPAAT1-2::PmHXT1-ScarMEL1-CvNR:PmSAD2-2 v2-AtPDCT-CvNR::PLSC-2/LPAAT1-2

pSZ5439 has the same vector backbone; selectable marker, promoters, and 3′ utr as pSZ5344, differing only in the genomic region used for construct targeting Relevant restriction sites in these constructs are also the same as in pSZ5344. The sequences of PLSC-2/LPAAT1-2 5′ flank, PLSC-2/LPAAT1-2 3′ flank used in pSZ5349 are shown below. Relevant restriction sites as bold text are shown 5′-3′ respectively.

PLSC-2/LPAAT1-2 5′ flank in pSZ5349:
(SEQ ID NO: 127)
gctcttctgcttcggattccactacatcaagtgggtgaacctggcggg
cgcggaggagggcccccgcccgggcggcattgttagcaaccactgcag
ctacctggacatcctgctgcacatgtccgactccttccccgcctttgt
ggcgcgccagtcgacggccaagctgccctttatcggcatcatcaggtg
cgtgaaagcgggggctgctgtggccgtggtgggcagggttgcgaaggg
gggcaggcgtaggcgtgcagtgtgagcggacattgatgccgtcgtttg
ccggtcaggagagctcgaaatcagagccagcctggtcatgggatcaca
gagctcaccaccactcgtccacctcgccttgccttgcagccaaatcat
gagggcctctacgtgaaccgcgaccgctcggggcccaaccacgtgggc
gtggccgatctggtgaagcagcgcatgcaggacgaggccgaggggagg
accccgcccgagtaccgaccgctgctcctcttccccgaggtgggcttt
cgaggcaccgtttgtgcttgaaactgtgggcacgcgtgccccgacgcg
cctctggcgcctgcttcgcatccattcgcctctcaaccccgtctctcc
tttcctccatcgccagggcaccacctccaacggcgactacctgcttcc
cttcaagaccggcgccttcctggccggggtgcccgtccagcccgtggt
acc
PLSC-2/LPAAT1-2 3′ flank in pSZ5349.
(SEQ ID NO: 128)
gagctccgtcctccactaccacagggtatggtggtgtggggtcgagcg
tgttgaagcgcggaaggggatgcgctgtcaagttttggagctgaaaat
ggtgcccgcgaggatccagcgcgccccactcacccttgctgccatcgc
tccccaccatttccccagggaaccctgtggcccacgtgggagacgatt
ccggccaagtggcacatcttcctgatgctctgccacccccgccacaaa
gtgaccgtgatgaaggtacgaacaagggtcgggccccgattctggata
tcacgtctggggtgtgtttctcgcgcacgcgtcccccgatgcgctgca
cagtctccctcacaccctcacccctaacgctcgcagttgcccgtgtac
gtccccaatgaggaggaaaaggccgaccccaagctgtacgcccaaaat
gttcgcaaagccatggtgcgtcgggaaccgttcaagtttgcttgcggg
tgggcggggcggctctagcgaattggcgcattggccctcaccgaggca
gcacatcggacaccaatcgtcacccggcgagcaattccgccccctctg
tcttctcgcagatggaggtcgccgggaccaaggacacgacggcggtgt
ttgaggacaagatgcgctacctgaactccctgaagagaaagtacggca
agcctgtgcctaagaaaattgagtgaacccccgtcgtcgaccagaaga
gc

To determine their impact on fatty acid profiles, both the constructs described above were transformed independently into S7211 and S8028. Primary transformants were clonally purified and grown under standard lipid production conditions at pH7.0. As discussed above, S7211 expresses a FAE, from C. abyssinica under the control of pH regulated, PMSAD2V-2(Ammonium transporter 03) promoter. Thus both parental (S7211) and the resulting PDCT transformed strains require growth at pH 7.0 to allow for maximal fatty acid elongase (FAE) gene expression.

S8028 and its derivative lines transformed with AtPDCT were cultured at pH 5.0. The resulting profiles from a set of representative clones arising from transformations with pSZ5344 (D4205) and pSZ5349 (D4210) into S7211 and S8028 are shown in Tables 85-88 respectively.

The expectation with the expression of PDCT into our algal host was somewhat increased C18:2 and/or VLCFA (in S7211) since our host has a moderate LPCAT activity which normally results in 5-7% C18:2 in our base strains. However contrary to our expectation there was more than 2.5 fold increase in C18:2 levels in strains expressing PDCT at either PLSC-2/LPAAT1-1 or PLSC-2/LPAAT1-2 genomic locus in both S7211 and S8028. In the best case scenario the increase in C18:2 level was 2.85 fold in S7211; T1181; D4210-10; pH7 over the parent (27.12 vs 9.53% in parent S7211) and 3.19 fold in S8028; T1226; D4205-1; pH5 (18.76% vs 5.88% in parent S8028). PDCT expression also led to noticeable increase in C22:1 levels in S7211. In the best case scenario C22:1 increased from 1.36% in parent to 5.04% in S7211; T1181; D4210-10; pH7—an increase of 3.7 fold.

The increase in C18:2 in PDCT expressing lines reported herein is even more pronounced than when higher plant LPCAT genes are expressed in S7211 (reported earlier). LPCAT overexpression leads to increased conversion of C18:1-CoA into C18:1-PC which then becomes available for further desaturation and/or elongation by competing FAD2 and FAE enzyme activities respectively. Since PDCT efficiently removes the PC associated polyunsaturated fatty acids for eventual incorporation into DAG pool, our results strongly suggest that the PC to DAG conversion by endogenous DAG-CPT in our host is somewhat inefficient. This inefficiency is removed by transplanting a higher plant PDCT gene into our algal genome. Furthermore once an efficient PC to DAG conversion is set into place by expression of AtPDCT, this likely increases the efficiency of upstream endogenous PmLPCAT enzyme and results in increased conversion of C18:1-CoA to C18:1-PC. At this stage it is unclear whether the elongation by CrhFAE occurs on the C18:1-PC (as opposed to C18:1-CoA) since PmFAD2-1 seems to compete better for the substrate than CrhFAE. Expressing CrhFAE and AtPDCT in a strain with very low FAD2 activity will help to understand the relation between desaturation and elongation in our algal host.

In summary, identification of LPCAT (discussed above) and now AtPDCT enzymes to increase conversion of C18:1 to C18:1-PC gives us a much better control over C18:1 phospholipid pool which can then be either directed towards making more polyunsaturated fatty acids or VLCFA by modulating the PmFAD2-1 activity.

TABLE 85
Unsaturated fatty acid profile in S3150, S7211 and
representative derivative transgenic lines transformed
with pSZ5344 (AtPDCT at PLSC-2/LPAAT1-1 genomic
locus) DNA.

| | | | | Sum | |
|---|---|---|---|---|---|
| Sample ID | C18:1 | C18:2 | C18:3 a | C20:1 | C22:1 |
| S7211; T1181; | 30.03 | 24.05 | 1.23 | 4.88 | 2.44 |
| D4205-9; pH 7 | | | | | |
| S7211; T1181; | 31.20 | 24.32 | 1.04 | 5.04 | 2.36 |
| D4205-1; pH 7 | | | | | |
| S7211; T1181; | 34.96 | 22.05 | 0.86 | 5.52 | 2.16 |
| D4205-8; pH 7 | | | | | |
| S7211; T1181; | 31.66 | 23.97 | 0.98 | 5.47 | 2.15 |
| D4205-6; pH 7 | | | | | |
| S7211; T1181; | 26.92 | 24.51 | 0.99 | 4.61 | 2.11 |
| D4205-18; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 86
Unsaturated fatty acid profile in S3150, S7211 and representative
derivative transgenic lines transformed with pSZ5349 (AtPDCT at
PLSC-2/LPAAT1-2 genomic locus) DNA.

| | | | | Sum | |
|---|---|---|---|---|---|
| Sample ID | C18:1 | C18:2 | C18:3a | C20:1 | C22:1 |
| S7211; T1181; | 23.16 | 27.15 | 1.73 | 6.33 | 5.04 |
| D4210-10; pH 7 | | | | | |
| S7211; T1181; | 23.81 | 26.10 | 1.55 | 6.01 | 4.19 |
| D4210-19; pH 7 | | | | | |
| S7211; T1181; | 26.74 | 26.00 | 1.47 | 5.78 | 3.90 |
| D4210-12; pH 7 | | | | | |
| S7211; T1181; | 31.12 | 24.49 | 1.22 | 4.99 | 2.59 |
| D4210-11; pH 7 | | | | | |
| S7211; T1181; | 32.16 | 24.01 | 1.19 | 5.07 | 2.42 |
| D4210-14; pH 7 | | | | | |
| S7211; pH 7 | 47.76 | 9.53 | 0.74 | 4.05 | 1.37 |
| S7211; pH 7 | 47.73 | 9.53 | 0.79 | 4.02 | 1.36 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 87
Unsaturated fatty acid profile in S8028 and representative
derivative transgenic lines transformed with pSZ5344 (AtPDCT
at PLSC-2/LPAAT1-1 genomic locus) DNA.

| | | | | Sum | |
|---|---|---|---|---|---|
| Sample ID | C18:1 | C18:2 | C18:3a | C20:1 | C22:1 |
| S8028; T1226; | 54.19 | 18.76 | 0.71 | 0.12 | 0.00 |
| D4205-1; pH 5 | | | | | |
| S8028; T1226; | 56.14 | 18.22 | 0.79 | 0.19 | 0.00 |
| D4205-47; pH 5 | | | | | |
| S8028; T1226; | 57.98 | 16.79 | 0.56 | 0.11 | 0.00 |
| D4205-48; pH 5 | | | | | |
| S8028; T1226; | 57.93 | 16.78 | 0.61 | 0.13 | 0.00 |
| D4205-5; pH 5 | | | | | |
| S8028; T1226; | 57.39 | 16.31 | 0.57 | 0.15 | 0.00 |
| D4205-20; pH 5 | | | | | |
| S8028 (pH 5); pH 5 | 68.13 | 5.88 | 0.54 | 0.11 | 0.00 |
| S8028 (pH 5); pH 5 | 68.08 | 5.85 | 0.54 | 0.15 | 0.00 |

TABLE 88
Unsaturated fatty acid profile in S8028 and representative
derivative transgenic lines transformed with pSZ5349 (AtPDCT
at PLSC-2/LPAAT1-2 genomic locus) DNA.

| | | | | Sum | |
|---|---|---|---|---|---|
| Sample ID | C18:1 | C18:2 | C18:3a | C20:1 | C22:1 |
| S8028; T1226; | 54.61 | 17.53 | 0.85 | 0.16 | 0.00 |
| D4210-34; pH 5 | | | | | |
| S8028; T1226; | 58.43 | 17.43 | 0.50 | 0.18 | 0.00 |
| D4210-7; pH 5 | | | | | |
| S8028; T1226; | 51.95 | 17.00 | 0.60 | 0.11 | 0.00 |
| D4210-20; pH 5 | | | | | |
| S8028; T1226; | 55.65 | 16.74 | 0.77 | 0.19 | 0.00 |
| D4210-14; pH 5 | | | | | |
| S8028; T1226; | 56.42 | 16.72 | 0.65 | 0.18 | 0.00 |
| D4210-3; pH 5 | | | | | |
| S8028 (pH 5); pH 5 | 68.13 | 5.88 | 0.54 | 0.11 | 0.00 |
| S8028 (pH 5); pH 5 | 68.08 | 5.85 | 0.54 | 0.15 | 0.00 |

### Example 14

### Expression of PDCT in a High-Linolenic Transgenic Microalga

In this example we demonstrate using Arabidopsis thaliana Phosphatidylcholine diacylglycerol cholinephosphotransferase (AtPDCT) gene to alter the content and composition of oils in transgenic algal strains for producing oils rich in linoleic and/or linolenenic acids.

We determined the effect of AtPDCT expression on C18:3 levels in linolenic strain S3709 expressing Linum usitatissimu FADS desaturase. S3709 was prepared according to Example 11 of co-owned application WO2012/106560. The sequence of AtPDCT was codon optimized for expression in our algal host and transformed into S3709.

Our results show that expression of AtPDCT in Solazyme linolenic strain S3709 results in more than 2 fold enhancement in linolenic acid (C18:3) content in individual lines over the parents.

Construct Used for the Expression of the A. thaliana Phosphatidylcholine Diacylglycerol Cholinephosphotransferase (AtPDCT) in Erucic Strain S3709 [pSZ5344]:

S3709, transformed with the construct pSZ5344, were generated which express Sacharomyces carlbergenesis MEL1 gene (allowing for their selection and growth on medium containing melibiose) and A. thaliana PDCT gene targeted at the endogenous PmLPAAT1-1 genomic region. Construct pSZ5344 introduced for expression in S7211 can be written as PLSC-2/LPAAT1-1 5′ flank::PmHXT1-ScarMEL1-CvNR:PmSAD2-2v2-AtPDCT-CvNR::PLSC-2/LPAAT1-1 3′ flank.

The sequence of the transforming DNA is provided below. Relevant restriction sites in the construct are indicated in lowercase, underlined bold, and are from 5′-3′ BspQI, KpnI, SpeI, SnaBI, EcoRI, SpeI, AflII, SacI, BspQI, respectively. BspQI sites delimit the 5′ and 3′ ends of the transforming DNA. Bold, lowercase sequences represent genomic DNA from S3150 that permit targeted integration at the PLSC-2/LPAAT1-1 locus via homologous recombination. Proceeding in the 5′ to 3′ direction, the endogenous P. moriformis Hexose Transporter 1 promoter driving the expression of the S. carlbergenesis MEL1 gene is indicated by lowercase, boxed text. The initiator ATG and terminator TGA for MEL1 are indicated by uppercase italics, while the coding region is indicated with lowercase italics. The Chlorella vulgaris nitrate reductase (NR) gene 3′ UTR is indicated by lowercase underlined text followed by a PMSAD2-v2 promoter of P. moriformis, indicated by boxed italicized text. The Initiator ATG and terminator TGA codons of the AtPDCT are indicated by uppercase, bold italics, while the remainder of the gene is indicated by bold italics. The C. vulgaris nitrate reductase 3′ UTR is again indicated by lowercase underlined text followed by the S3150 PLSC-2/LPAAT1-1 genomic region indicated by bold, lowercase text. The final construct was sequenced to ensure correct reading frames and targeting sequences.

Nucleotide sequence of transforming DNA contained in plasmid pSZ5344:
(SEQ ID NO: 129)
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccdttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg
gccggggtgcccgtccagcccgtggtaccgcggtgagaatcgaaaatgcatcgtttctaggttcggagacggtcaattccctgctcc
ggcgaatctglcgglcaagctggccagtggacaatgltgctatggcagcccgcgcacatgggcctcccgacgcggccatcaggagc
ccaaacagcgtgtcagggtatgtgaaactcaagaggtccctgctgggcactccggccccactccgggggcgggacgccaggcattc
gcggtcggtcccgcgcgacgagcgaaatgatgattcggttacgagaccaggacgtcgtcgaggtcgagaggcagcctcggacacg
tctcgctagggcaacgccccgagtccccgcgagggccgtaaacattgtttctgggtgtcggagtgggcattttgggcccgatccaatc
gcctcatgccgctctcgtctggtcctcacgttcgcgtacggcctggatcccggaaagggcggatgcacgtggtgttgccccgccattg
gcgcccacgtttcaaagtccccggccagaaatgcacaggaccggcccggctcgcacaggccatgctgaacgcccagatttcgaca
gcaacaccatctagaataatcgcaaccatccgcgttttgaacgaaacgaaacggcgctgtttagcatgtttccgacatcgtgggggccg
aagcatgctccggggggaggaaagcgtggcacagcggtagcccattctgtgccacacgccgacgaggaccaatccccggcatca

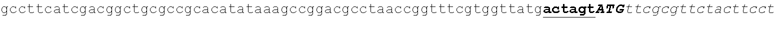

gacggcctgcatctccctgaagggcgtgttcggcgtctccccctcctacaacggcctgggcctgacgccccagatgggctgggac
aactggaacacgttcgcctgcgacgtctccgagcagctgctgctggacacggccgaccgcatctccgacctgggcctgaaggac
atgggctacaagtacatcatcctggacgactgctggtcctccggccgcgactccgacggcttcctggtcgccgacgagcagaagt
tccccaacggcatgggccacgtcgccgaccacctgcacaacaactccttcctgttcggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatccrgtacaacgccaccgagcagtcc
tacaaggacggcctgtccaagaacgacacccgcctgttcggccagaagatcggctccctgtcccccaacgcgatcctgaacacg

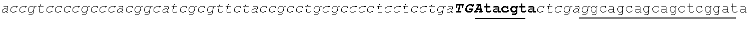

gtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtggg
atgggaacacaaatggaaagctgtagaattcctggctcgggcctcgtgctggcactccctcccatgccgacaacctttctgctgtcacc
acgacccacgatgcaacgcgacacgacccggtgggactgatcggttcactgcacctgcatgcaattgtcacaagcgcatactccaat
cgtatccgtttgatttctgtgaaaactcgctcgaccgcccgcgtcccgcaggcagcgatgacgtgtgcgtgacctgggtgttttcgtcga
aaggccagcaaccccaaatcgcaggcgatccggagattgggatctgatccgagcttggaccagatcccccacgatgcggcacggg
aactgcatcgactcggcgcggaacccagctttcgtaaatgccagattggtgtccgataccttgatttgccatcagcgaaacaagacttca
gcagcgagcgtatttggcgggcgtgctaccagggttgcatacattgcccatttctgtctggaccgctttaccggcgcagagggtgagtt
gatggggttggcaggcatcgaaacgcgcgtgcatggtgtgtgtgtctgttttcggctgcacaatttcaatagtcggatgggcgacggta
gaattgggtgttgcgctcgcgtgcatgcctcgccccgtcgggtgtcatgaccgggactggaatcccccctcgcgaccctcctgctaac

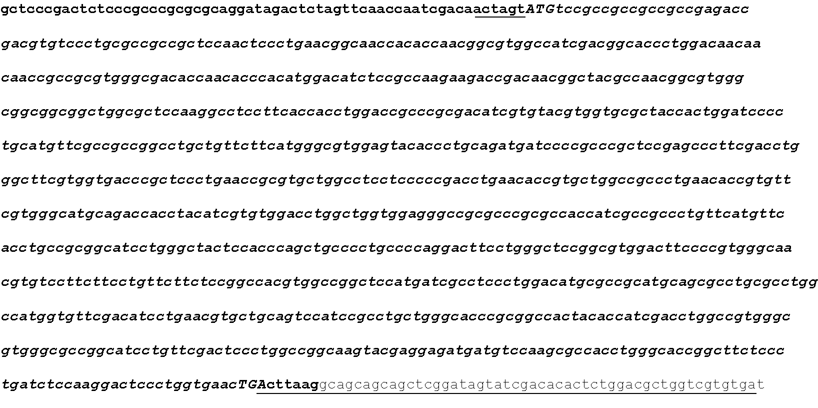

ggactgttgccgccacacttgctgccttgacctgtgaatatccctgccgcttttatcaaacagcctcagtgtgtttgatcttgtgtgtacgcg
cttttgcgagttgctagctgcttgtgctatttgcgaataccacccccagcatccccttccctcgtttcatatcgcttgcatcccaaccgcaac
ttatctacgctgtcctgctatccctcagcgctgctcctgctcctgctcactgcccctcgcacagccttggtttgggctccgcctgtattctcc
tggtactgcaacctgtaaaccagcactgcaatgctgatgcacgggaagtagtgggatgggaacacaaatggaaagcttaattaagag
ctccgtcctccactaccacagggtatggtcgtgtggggtcgagcgtgttgaagcgcagaaggggatgcgccgtcaagatcag
gagctaaaaatggtgccagcgaggatccagcgctctcactcttgctgccatcgctcccacccttttccccaggggaccctgtgg
cccacgtgggagacgattccggccaagtggcacatcttcctgatgctctgccacccccgccacaaagtgaccgtgatgaaggt
taggacaagggtcgggacccgattctggatatgacctctgaggtgtgtttctcgcgcaagcgtcccccaattcgttacaccaca
tccctcacaccctcgcccctgacactcgcagttgcccgtgtacgtccccaatgaggaggaaaaggccgaccccaagctgtacg
cccaaaacgtccgcaaagccatggtgcgtcgggaaccgtcaaagtttgcttgcgggtgggcggggcggctctagcgaattgg
ctcattggccctcaccgaggcagcacatcggacaccagtcgccacccggcttgcatcttcgccccctttcttctcgcagatggag
gtcgccgggaccaaggacacgacggcggtgtttgaggacaagatgcgctacctgaactccctgaagagaaagtacggcaag
cctgtgcctaagaaaattgagtgaacccccgtcgtcgatccagaagagc

In addition to the A. thaliana PDCT targeted at PLSC-2/PmLPAAT1-1 locus (pSZ5344), A. thaliana PDCT targeted at PLSC-2/LPAAT1-2 locus (pSZ5349), was constructed for expression in S7211. These constructs can be described as:

pSZ5349-PLSC-2/LPAAT1-2::PmHXT1-ScarMEL1-CvNR:PmSAD2-2v2-AtPDCT-CvNR::PLSC-2/LPAAT1-2

pSZ5439 has the same vector backbone; selectable marker, promoters, and 3′ utr as pSZ5344, differing only in the genomic region used for construct targeting Relevant restriction sites in these constructs are also the same as in pSZ5344. The sequence of PLSC-2/LPAAT1-2 5′ flank, PLSC-2/LPAAT1-2 3′ flank used in pSZ5344 are provided below. Relevant restriction sites as bold text are shown 5′-3′ respectively.

PLSC-2/LPAAT1-2 5′ flank in pSZ5349:
(SEQ ID NO: 130)
gctcttctgcttcggattccactacatcaagtgggtgaacctggcggg
cgcggaggagggccccgcccgggcggcattgttagcaaccactgcagc
tacctggacatcctgctgcacatgtccgactccttccccgcctttgtg
gcgcgccagtcgacggccaagctgccctttatcggcatcatcaggtgc
gtgaaagcgggggctgctgtggccgtggtgggcagggttgcgaagggg
ggcaggcgtaggcgtgcagtgtgagcggacattgatgccgtcgtttgc
cggtcaggagagctcgaaatcagagccagcctggtcatgggatcacag
agctcaccaccactcgtccacctcgcctgccttgcagccaaatcatga
gctgcctctacgtgaaccgcgaccgctcggggcccaaccacgtgggcg
tggccgatctggtgaagcagcgcatgcaggacgaggccgaggggagga
ccccgcccgagtaccgaccgctgctcctcttccccgaggtgggctttc
gaggcaccgtttgtgcttgaaactgtgggcacgcgtgccccgacgcgc
ctctggcgcctgcttcgcatccattcgcctctcaaccccgtctctcct
ttcctccatcgccagggcaccacctccaacggcgactacctgcttccc
ttcaagaccggcgccttcctggccggggtgcccgtccagcccgtggta
cc
PLSC-2/LPAAT1-2 3′ flank in pSZ5349:
(SEQ ID NO: 131)
gagctccgtcctccactaccacagggtatggtggtgtggggtcgagcg
tgttgaagcgcggaaggggatgcgctgtcaagttttggagctgaaaat
ggtgcccgcgaggatccagcgcgccccactcacccttgctgccatcgc
tccccacccttttccccagggaaccctgtggcccacgtgggagacgat
tccggccaagtggcacatcttcctgatgctctgccacccccgccacaa
agtgaccgtgatgaaggtacgaacaagggtcgggccccgattctggat
atcacgtctggggtgtgtttctcgcgcacgcgtcccccgatgcgctgc
acagtctccctcacaccctcacccctaacgctcgcagttgcccgtgta
cgtccccaatgaggaggaaaaggccgaccccaagctgtacgcccaaaa
tgttcgcaaagccatggtgcgtcgggaaccgttcaagtttgcttgcgg
gtgggcggggcggctctagcgaattggcgcattggccctcaccgaggc
agcacatcggacaccaatcgtcacccggcgagcaattccgccccctct
gtcttctcgcagatggaggtcgccgggaccaaggacacgacggcggtg
tttgaggacaagatgcgctacctgaactccctgaagagaaagtacggc
aagcctgtgcctaagaaaattgagtgaacccccgtcgtcgaccagaag
agc

To determine their impact on fatty acid profiles, both the constructs described above were transformed independently into S3709. Primary transformants were clonally purified and grown under standard lipid production conditions at pH7.0. S3709 expresses a LnFAD3, from Linum usitatissimu under the control of pH regulated, PMSAD2-v2(Ammonium transporter 03) promoter. Thus both parental (S3709) and the resulting PDCT transformed strains require growth at pH 7.0 to allow for maximal fatty acid desaturase (LnFAD3) gene expression. The resulting profiles from a set of representative clones arising from transformations with pSZ5344 (D4205) and pSZ5349 (D4210) into S3709 are shown in Tables 89 and 90, respectively.

Individual transgenic lines expressing AtPDCT genes resulted in more than 2 fold increase in C18:3 (Tables 89 and 90). The increase in C18:3 in S3709; T1228; D4205-36; pH7 12.17 fold (14.51%) while the increase was 1.89 fold in S3709; T1228; D4210-4; pH7 (12.61%) over the parent S3709 (6.66%). As discussed in Example 13 above, enhancing the removal of PC associated polyunsaturated fatty acids by AtPDCT increases the C18:2 content more than just expressing a heterologous PDCT in our host. However, unlike the S3709 parent, not all of the available C18:2 is converted into C18:3. This is most likely due to sub-optimal expression of LnFAD3 in S3709.

Since both LPCAT and PDCT enzymes channel polyunsaturates onto DAG, it would be informative to combine these two activities together and express them in various background strains like S3709 (Linolenic strain), S8028 (High Oleic base strain) or S7211 (Erucic strain).

TABLE 89
Unsaturated fatty acid profile in S3709 and representative derivative
transgenic lines transformed with pSZ5344 (AtPDCT at PLSC-2/
LPAAT1-1 genomic locus) DNA.

| Sample ID | 14:0 | 16:0 | 18:0 | 18:1 | 18:2 | 18:3 a |
|---|---|---|---|---|---|---|
| S3709 (pH 7); pH 7 | .86 | 8.85 | .54 | 7.22 | .42 | .66 |
| S3709 (pH 7); pH 7 | .90 | 9.00 | .54 | 6.89 | .45 | .81 |
| S3709; T1228; D4205-36; | .62 | 2.74 | .48 | 8.67 | .12 | 4.51 |
| pH 7 | | | | | | |
| S3709; T1228; D4205-1; | .94 | 7.62 | .57 | 5.09 | .28 | 1.53 |
| pH 7 | | | | | | |
| S3709; T1228; D4205-4; | .42 | 9.48 | .15 | 3.03 | 0.91 | 0.22 |
| pH 7 | | | | | | |
| S3709; T1228; D4205-44; | .80 | 8.81 | .53 | 2.84 | .18 | .20 |
| pH 7 | | | | | | |
| S3709; T1228; D4205-33; | .06 | 1.79 | .75 | 2.21 | .07 | .17 |
| pH 7 | | | | | | |

TABLE 90
Unsaturated fatty acid profile in S3709 and representative derivative
transgenic lines transformed with pSZ5349 (AtPDCT at PLSC-2/
LPAAT1-2 genomic locus) DNA.

| Sample ID | 14:0 | 16:0 | 18:0 | 18:1 | 18:2 | 18:3 a |
|---|---|---|---|---|---|---|
| S3709 (pH 7); pH 7 | .86 | 8.85 | .54 | 7.22 | .42 | .66 |
| S3709 (pH 7); pH 7 | .90 | 9.00 | .54 | 6.89 | .45 | .81 |
| S3709; T1228; D4210-4; | .11 | 6.68 | .59 | 0.05 | .00 | 2.61 |
| pH 7 | | | | | | |
| S3709; T1228; D4210-36; | .97 | 9.44 | .85 | 5.40 | .67 | 1.93 |
| pH 7 | | | | | | |
| S3709; T1228; D4210-11; | .92 | 7.35 | .53 | 8.82 | .19 | 0.98 |
| pH 7 | | | | | | |
| S3709; T1228; D4210-38; | .18 | 9.20 | .36 | 5.08 | .82 | .25 |
| pH 7 | | | | | | |
| S3709; T1228; D4210-43; | .97 | 8.81 | .47 | 6.38 | .57 | .21 |
| pH 7 | | | | | | |

### Example 15

### Expression of DAG-CPT in a High-Erucic Transgenic Microalga

In this example we demonstrate using higher plant CDP-choline:1,2-sn-diacylglycerol cholinephosphotransferase (DAG-CPT) gene to alter the content and composition of oils in transgenic algal strains for producing oils rich in linoleic and/or very long chain fatty acids (VLCFA).

We used A. thaliana AtDAG-CPT (NP_172813) available in the public databases to identify corresponding DAG-CPT genes from our internally assembled transcriptomes of B. rapa, and B. juncea. The codon optimized sequences of all the internally identified genes (BrDAG-CPT and BjDAG-CPT), along with AtDAG-CPT genes, were expressed in strain S7211. The preparation of S7211 is discussed above.

Our results show that expression of DAG-CPT genes in Solazyme erucic strain S7211 results in enhancement in linoleic (C18:2) and erucic (C22:1) acid content in individual lines over the parents.

Construct Used for the Expression of the A. thaliana Phosphatidylcholine Diacylglycerol Cholinephosphotransferase (AtDAG-CPT) in Erucic Strain S7211 [pSZ5295]:

In this example, transgenic lines from S7211, transformed with the construct pSZ5295, were generated. These lines express Sacharomyces carlbergenesis MEL1 gene and A. thaliana DAG-CPT gene targeted at endogenous PmLPAAT1-1 genomic region. Construct pSZ5295 introduced for expression in S7211 can be written as PLSC-2/LPAAT1-1 5′ flank::PmHXT1-ScarMEL1-CvNR:PmSAD2-2v2-AtDAG-CPT-CvNR::PLSC-2/LPAAT1-1 3′ flank.

The sequence of the transforming DNA is provided in below. Relevant restriction sites in the construct are indicated in lowercase, underlined bold, and are from 5′-3′ BspQI, KpnI, SpeI, SnaBI, EcoRI, SpeI, AflII, SacI, BspQI, respectively. BspQI sites delimit the 5′ and 3′ ends of the transforming DNA. Bold, lowercase sequences represent genomic DNA from S3150 that permit targeted integration at the PLSC-2/LPAAT1-1 locus via homologous recombination. Proceeding in the 5′ to 3′ direction, the endogenous P. moriformis Hexose Transporter 1 promoter driving the expression of the S. carlbergenesis MEL1 gene is indicated by lowercase, boxed text. The initiator ATG and terminator TGA for MEL1 are indicated by uppercase italics, while the coding region is indicated with lowercase italics. The Chlorella vulgaris nitrate reductase (NR) gene 3′ UTR is indicated by lowercase underlined text followed by a PMSAD2-v2 promoter of P. moriformis, indicated by boxed italicized text. The Initiator ATG and terminator TGA codons of the AtDAG-CPT are indicated by uppercase, bold italics, while the remainder of the gene is indicated by bold italics. The C. vulgaris nitrate reductase 3′ UTR is again indicated by lowercase underlined text followed by the S3150 PLSC-2/LPAAT1-1 genomic region indicated by bold, lowercase text. The final construct was sequenced to ensure correct reading frames and targeting sequences.

Nucleotide sequence of transforming DNA contained in plasmid pSZ5295:
(SEQ ID NO: 132)
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccctttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg
gccggggtgcccgtccagcccgtggtaccgcggtgagaatcgaaaatgcatcgtactaggacggagacggtcaattccctgctcc
ggcgaatctgtcggtcaagctggccagtggacaatgttgctatggcagcccgcgcacatgggcctcccgacgcggccatcaggagc
ccaaacagcgtgtcagggtatgtgaaactcaagaggtccctgctgggcactccggccccactccgggggcgggacgccaggcattc
gcggtcggtcccgcgcgacgagcgaaatgatgattcggttacgagaccaggacgtcgtcgaggtcgagaggcagcctcggacacg
tctcgctagggcaacgccccgagtccccgcgagggccgtaaacattgtttctgggtgtcggagtgggcattttgggcccgatccaatc
gcctcatgccgctctcgtctggtcctcacgttcgcgtacggcctggatcccggaaagggcggatgcacgtggtgttgccccgccattg
gcgcccacgtttcaaagtccccggccagaaatgcacaggaccggcccggctcgcacaggccatgctgaacgcccagatttcgaca
gcaacaccatctagaataatcgcaaccatccgcgttttgaacgaaacgaaacggcgctgtttagcatgtttccgacatcgtgggggccg
aagcatgctccggggggaggaaagcgtggcacagcggtagcccattctgtgccacacgccgacgaggaccaatccccggcatca

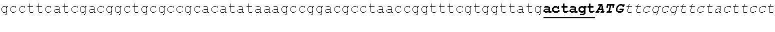

gacggcctgcatctccctgaagggcgtgttcggcgtctccccctcctacaacggcctgggcctgacgccccagatgggctgggac
aactggaacacgttcgcctgcgacgtctccgagcagctgctgctggacacggccgaccgcatctccgacctgggcctgaaggac
atgggctacaagtacatcatcctggacgactgcggtcctccggccgcgactccgacggcttcctggtcgccgacgagcagaagt
tccccaacggcatgggccacgtcgccgaccacctgcacaacaactccttcctgttcggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctggaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgttcggccagaagatcggctccctgtcccccaacgcgatcctgaacacg

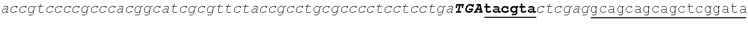

gtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtggg
atgggaacacaaatggaaagctgtagaattcctggctcgggcctcgtgctggcactccctcccatgccgacaacctttctgctgtcacc
acgacccacgatgcaacgcgacacgacccggtgggactgatcggttcactgcacctgcatgcaattgtcacaagcgcatactccaat
cgtatccgtttgatttctgtgaaaactcgctcgaccgcccgcgtcccgcaggcagcgatgacgtgtgcgtgacctgggtgtttcgtcga
aaggccagcaaccccaaatcgcaggcgatccggagattgggatctgatccgagcttggaccagatcccccacgatgcggcacggg
aactgcatcgactcggcgcggaacccagctttcgtaaatgccagattggtgtccgataccttgatttgccatcagcgaaacaagacttca
gcagcgagcgtatttggcgggcgtgctaccagggttgcatacattgcccatttctgtctggaccgctttaccggcgcagagggtgagtt
gatggggttggcaggcatcgaaacgcgcgtgcatggtgtgtgtgtctgttttcggctgcacaatttcaatagtcggatgggcgacggta
gaattgggtgttgcgctcgcgtgcatgcctcgccccgtcgggtgtcatgaccgggactggaatcccccctcgcgaccctcctgctaac

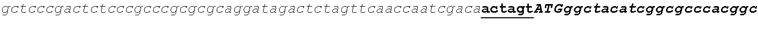

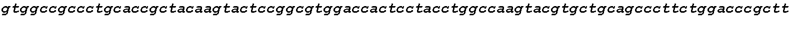

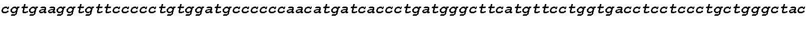

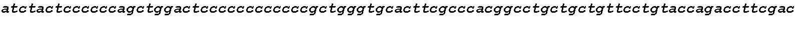

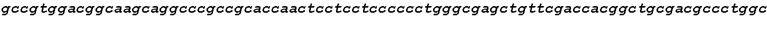

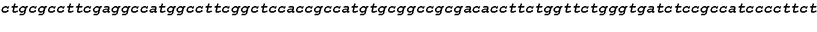

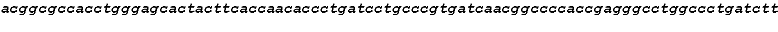

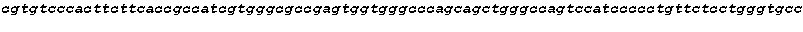

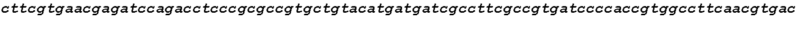

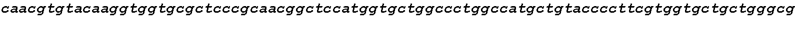

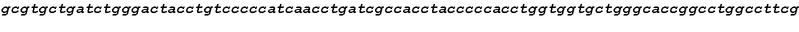

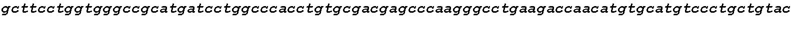

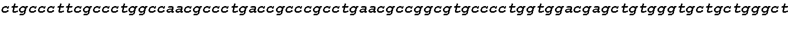

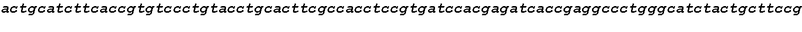

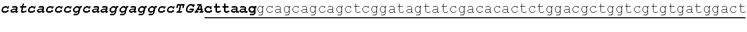

gttgccgccacacttgctgccttgacctgtgaatatccctgccgcttttatcaaacagcctcagtgtgtttgatcttgtgtgtacgcgcttttg
cgagagctagctgcagtgctatttgcgaataccacccccagcatccccaccctcgatcatatcgcagcatcccaaccgcaacttatct
acgctgtcctgctatccctcagcgctgctcctgctcctgctcactgcccctcgcacagccttggtttgggctccgcctgtattctcctggta
ctgcaacctgtaaaccagcactgcaatgctgatgcacgggaagtagtgggatgggaacacaaatggaaagcttaattaagagctccg
tcctccactaccacagggtatggtcgtgtggggtcgagcgtgttgaagcgcagaaggggatgcgccgtcaagatcaggagct
aaaaatggtgccagcgaggatccagcgctctcactcttgctgccatcgctcccaccatttccccaggggaccctgtggcccac
gtgggagacgattccggccaagtggcacatcttcctgatgactgccacccccgccacaaagtgaccgtgatgaaggttagga
caagggtcgggacccgattctggatatgacctctgaggtgtgtttctcgcgcaagcgtcccccaattcgttacaccacatccctc
acaccctcgcccctgacactcgcagttgcccgtgtacgtccccaatgaggaggaaaaggccgaccccaagagtacgcccaa
aacgtccgcaaagccatggtgcgtcgggaaccgtcaaagtttgcttgcgggtgggcggggcggctctagcgaattggctcatt
ggccctcaccgaggcagcacatcggacaccagtcgccacccggcttgcatcttcgccccattcttctcgcagatggaggtcgc
cgggaccaaggacacgacggcggtgtttgaggacaagatgcgctacctgaactccctgaagagaaagtacggcaagcctgt
gcctaagaaaattgagtgaacccccgtcgtcgaccagaagagc

Constructs Used for the Expression of the AtDAG-CPT, BjDAG-CPT and BrDAG-CPT at PLSC-2/PmLPAAT1-1 or PLSC-2/PmLPAAT1-2 loci in S7211:

In addition to the A. thaliana DAG-CPT targeted at PLSC-2/PmLPAAT1-1 locus (pSZ5295), A. thaliana DAG-CPT targeted at PLSC-2/LPAAT1-2 locus (pSZ5305), BrDAG-CPT targeted at PLSC-2/PmLPAAT1-1 locus (pSZ5345), BrDAG-CPT targeted at PLSC-2/PmLPAAT1-2 locus (pSZ5350), BjDAG-CPT targeted at PLSC-2/PmLPAAT1-1 locus (pSZ5347) and BjDAG-CPT targeted at PLSC-2/PmLPAAT1-2 locus (pSZ5306), have been constructed for expression in S7211. These constructs can be described as:

pSZ5305 PLSC-2/LPAAT1-2::PmHXT1-ScarMEL1-CvNR:PmSAD2-2 v2-AtDAG-CPT-CvNR::PLSC-2/LPAAT1-2
pSZ5345 PLSC-2/LPAAT1-1::PmHXT1-ScarMEL1-CvNR:PmSAD2-2 v2-BrDAG-CPT-CvNR::PLSC-2/LPAAT1-1
pSZ5306 PLSC-2/LPAAT1-2::PmHXT1-ScarMEL1-CvNR:PmSAD2-2 v2-BjDAG-CPT-CvNR::PLSC-2/LPAAT1-2
pSZ5347 PLSC-2/LPAAT1-1::PmHXT1-ScarMEL1-CvNR:PmSAD2-2 v2-BjDAG-CPT-CvNR::PLSC-2/LPAAT1-1
pSZ5350 PLSC-2/LPAAT1-2::PmHXT1-ScarMEL1-CvNR:PmSAD2-2 v2-BrDAG-CPT-CvNR::PLSC-2/LPAAT1-2

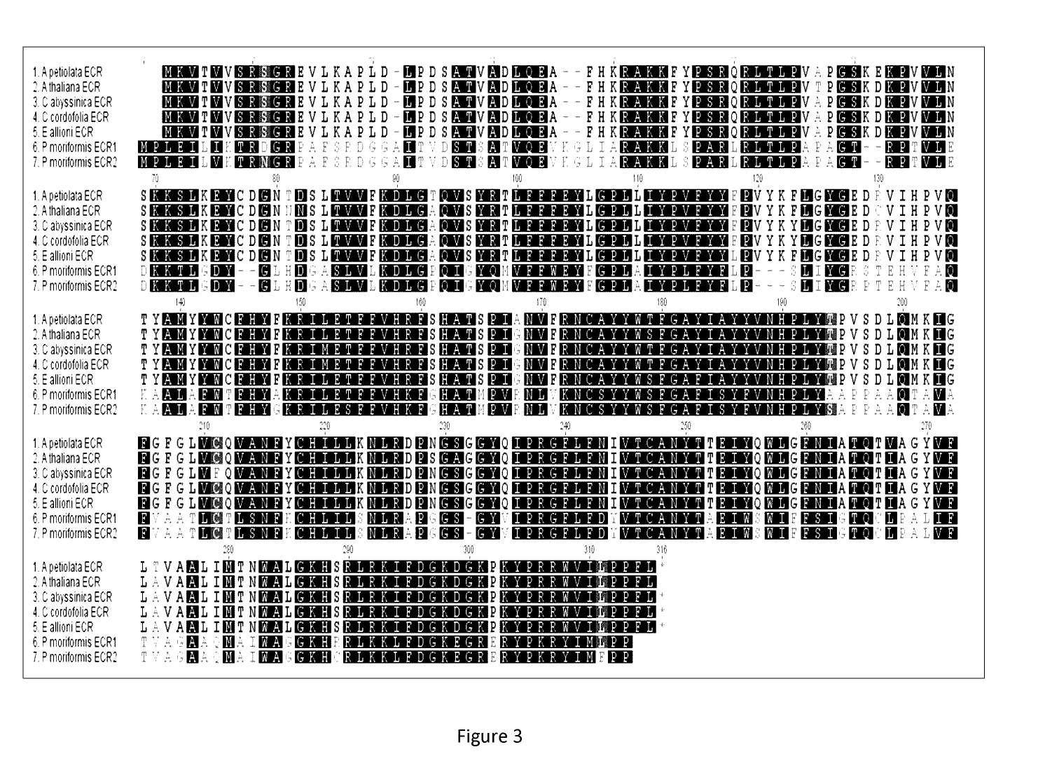

All these constructs have same vector backbone; selectable marker, promoters, and 3′ utr as pSZ5295, differing only in the genomic region used for construct targeting and/or the relevant DAG-CPT gene. Relevant restriction sites in these constructs are also same as in pSZ5295. FIGS. 3-6 indicate the sequence of PLSC-2/LPAAT1-2 5′ flank, PLSC-2/LPAAT1-2 3′ flank and BrDAG-CPT and BjDAG-CPT genes respectively. Relevant restriction sites as bold text are shown 5′-3′ respectively.

PLSC-2/LPAAT1-2 5′ flank in pSZ5305, pSZ5306 and pSZ5350:
(SEQ ID NO: 133)
gctcttctgcttcggattccactacatcaagtgggtgaacctggcgggcgcggaggagggcccccgcccgggcggcattgtta
gcaaccactgcagctacctggacatcctgctgcacatgtccgactccttccccgcctttgtggcgcgccagtcgacggccaagc
tgccctttatcggcatcatcaggtgcgtgaaagcgggggctgctgtggccgtggtgggcagggttgcgaaggggggcaggcg
taggcgtgcagtgtgagcggacattgatgccgtcgtttgccggtcaggagagctcgaaatcagagccagcctggtcatgggat
cacagagctcaccaccactcgtccacctcgcctgcgccttgcagccaaatcatgagctgcctctacgtgaaccgcgaccgctc
ggggcccaaccacgtgggcgtggccgatctggtgaagcagcgcatgcaggacgaggccgaggggaggaccccgcccgagt
accgaccgctgctcctcttccccgaggtgggctttcgaggcaccgtttgtgcttgaaactgtgggcacgcgtgccccgacgcgc
ctctggcgcctgcttcgcatccattcgcctctcaaccccgtctctcctttcctccatcgccagggcaccacctccaacggcgacta
cctgcttcccttcaagaccggcgccttcctggccggggtgcccgtccagcccgtggtacc
PLSC-2/LPAAT1-2 3′ flank in pSZ5305, pSZ5306 and pSZ5350:
(SEQ ID NO: 134)
gagctccgtcctccactaccacagggtatggtggtgtggggtcgagcgtgttgaagcgcggaaggggatgcgctgtcaagttt
tggagctgaaaatggtgcccgcgaggatccagcgcgccccactcacccttgctgccatcgctccccacccttttccccagggaa
ccctgtggcccacgtgggagacgattccggccaagtggcacatcttcctgatgctctgccacccccgccacaaagtgaccgtg
atgaaggtacgaacaagggtcgggccccgattctggatatcacgtctggggtgtgtttctcgcgcacgcgtcccccgatgcgct
gcacagtctccctcacaccctcacccctaacgctcgcagttgcccgtgtacgtccccaatgaggaggaaaaggccgaccccaa
gctgtacgcccaaaatgttcgcaaagccatggtgcgtcgggaaccgttcaagtttgcttgcgggtgggcggggcggctctagc
gaattggcgcattggccctcaccgaggcagcacatcggacaccaatcgtcacccggcgagcaattccgccccctagtcttctc
gcagatggaggtcgccgggaccaaggacacgacggcggtgtttgaggacaagatgcgctacctgaactccctgaagagaaa
gtacggcaagcctgtgcctaagaaaattgagtgaacccccgtcgtcgaccagaagagc
Sequence of BrDAG-CPT in pSZ5345 and pSZ5350:
(SEQ ID NO: 135)

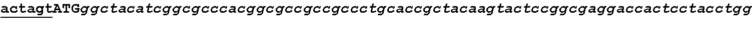

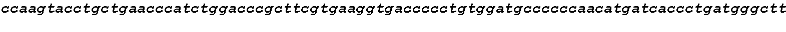

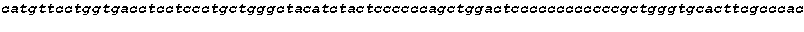

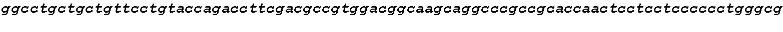

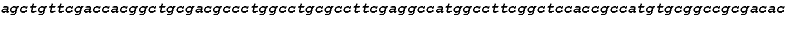

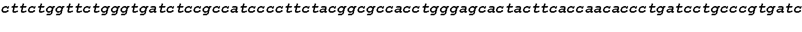

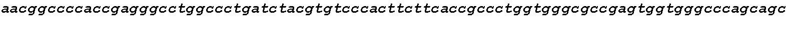

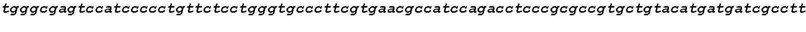

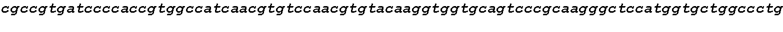

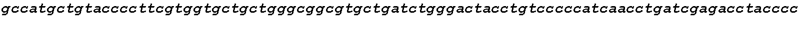

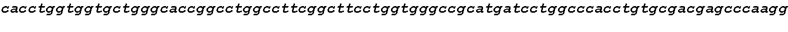

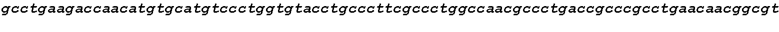

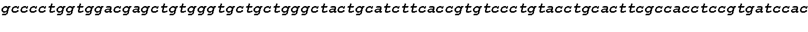

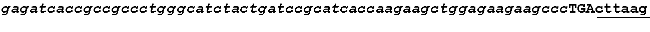

Sequence of BjDAG-CPT in pSZ5306 and pSZ5347:
(SEQ ID NO: 136)

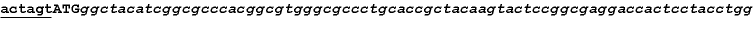

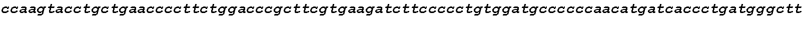

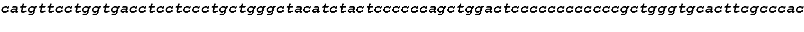

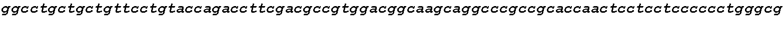

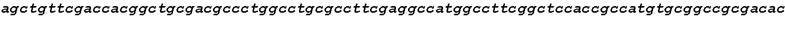

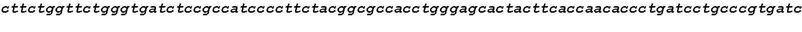

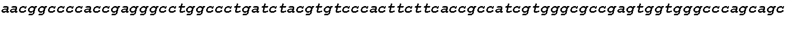

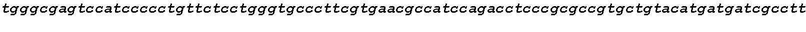

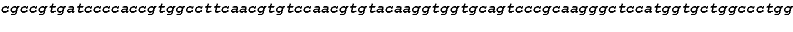

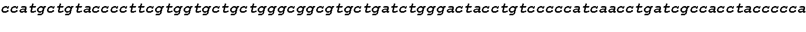

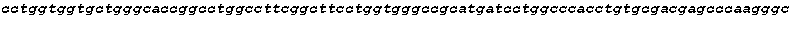

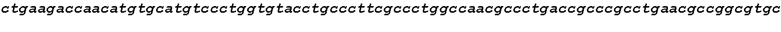

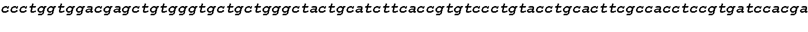

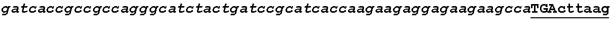

To determine their impact on fatty acid profiles, all the constructs described above were transformed independently into S7211. Primary transformants were clonally purified and grown under standard lipid production conditions at pH7.0. The resulting fatty acid profiles from a set of representative clones arising from transformations with pSZ5295 (D4156), pSZ5305 (D4166), pSZ5345 (D4206), pSZ5350 (D4211), pSZ5347 (D4208) and pSZ5306 (D4167) into S7211 sorted by C22:1 levels are shown in Tables 91-96, respectively.

The expectation was that the expression of DAG-CPTs into our algal host might enhance the removal of DAG-acyl-CoAs from PC and lead increase in polyunsaturated fatty and/or VLCFA in TAG since our host has a moderate LPCAT activity which normally results in 5-7% C18:2 in our base strains. We got noticeable and sustained increase in C18:2 and VLCFA levels in strains expression DAG-CPTs at either PLSC-2/LPAAT1-1 or PLSC-2/LPAAT1-2 genomic locus.

These results suggest that PC to DAG conversion by endogenous DAG-CPT in our host is somewhat inefficient and can be augmented by transplanting a corresponding higher plant homolog gene into our algal genome. Furthermore once an efficient PC to DAG conversion is set into place, this likely increases the efficiency of upstream endogenous PmLPCAT enzyme and results in increased conversion of C18:1-CoA to C18:1-PC.

In summary, identification of earlier discussed LPCAT and PDCT and DAG-CPT enzymes to increase conversion of C18:1 to C18:1-PC and their eventual removal from PC for incorporation into DAG gives us a much better control over C18:1 phospholipid pool which can then be either directed towards making more polyunsaturated fatty acids or VLCFA by modulating the PmFAD2-1 activity.

TABLE 91
Unsaturated fatty acid profile in S3150, S7211 and
representative derivative transgenic lines transformed
with pSZ5295 (AtDAG-CPT at PLSC-2/LPAAT1-1 genomic
locus) DNA.

| | | | | Sum | |
|---|---|---|---|---|---|
| Sample ID | C18:1 | C18:2 | C18:3a | C20:1 | C22:1 |
| S7211; T1172; | 37.45 | 15.68 | 1.26 | 6.18 | 4.16 |
| D4156-5; pH 7 | | | | | |
| S7211; T1172; | 39.25 | 15.00 | 1.20 | 5.77 | 3.47 |
| D4156-14; pH 7 | | | | | |
| S7211; T1172; | 41.78 | 13.04 | 1.29 | 5.80 | 3.43 |
| D4156-4; pH 7 | | | | | |
| S7211; T1172; | 38.61 | 15.68 | 1.40 | 6.02 | 3.30 |
| D4156-3; pH 7 | | | | | |
| S7211; T1172; | 39.80 | 14.61 | 1.16 | 5.61 | 3.27 |
| D4156-12; pH 7 | | | | | |
| S7211; pH 7 | 48.10 | 9.65 | 0.78 | 4.03 | 1.34 |
| S7211; pH 7 | 48.11 | 9.64 | 0.77 | 4.01 | 1.33 |
| S3150; pH 7 | 58 | 6.62 | 0.56 | 0.19 | 0 |
| S3150; pH 5 | 57.7 | 7.08 | 0.54 | 0.11 | 0 |

TABLE 92
Unsaturated fatty acid profile in S3150, S7211 and
representative derivative transgenic lines transformed
with pSZ5305 (AtDAG-CPT at PLSC-2/LPAAT1-2 genomic
locus) DNA.

| | | | | Sum | |
|---|---|---|---|---|---|
| Sample ID | C18:1 | C18:2 | C18:3a | C20:1 | C22:1 |
| S7211; T1173; | 38.33 | 15.16 | 1.53 | 5.64 | 3.33 |
| D4166-4; pH 7 | | | | | |
| S7211; T1173; | 37.99 | 16.12 | 1.32 | 5.53 | 3.19 |
| D4166-8; pH 7 | | | | | |
| S7211; T1173; | 39.17 | 14.89 | 1.41 | 5.54 | 3.07 |
| D4166-6; pH 7 | | | | | |
| S7211; T1173; | 38.71 | 15.11 | 1.38 | 5.45 | 2.99 |
| D4166-5; pH 7 | | | | | |
| S7211; T1173; | 39.75 | 14.34 | 1.37 | 5.36 | 2.99 |
| D4166-7; pH 7 | | | | | |
| S7211A; pH 7 | 48.23 | 9.69 | 0.75 | 4.02 | 1.34 |
| S7211B; pH 7 | 48.24 | 9.65 | 0.75 | 4.01 | 1.33 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 93
Unsaturated fatty acid profile in S3150, S7211 and
representative derivative transgenic lines transformed
with pSZ5345 (BrDAG-CPT at PLSC-2/LPAAT1-1 genomic
locus) DNA.

| | | | | Sum | |
|---|---|---|---|---|---|
| Sample ID | C18:1 | C18:2 | C18:3a | C20:1 | C22:1 |
| S7211; T1181; | 47.43 | 11.53 | 0.85 | 4.63 | 1.76 |
| D4206-13; pH 7 | | | | | |
| S7211; T1181; | 45.60 | 12.37 | 0.85 | 4.49 | 1.71 |
| D4206-15; pH 7 | | | | | |
| S7211; T1181; | 47.66 | 11.26 | 0.89 | 4.36 | 1.66 |
| D4206-12; pH 7 | | | | | |
| S7211; T1181; | 46.38 | 11.51 | 0.91 | 4.44 | 1.65 |
| D4206-5; pH 7 | | | | | |
| S7211; T1181; | 46.22 | 12.73 | 0.58 | 4.43 | 1.65 |
| D4206-7; pH 7 | | | | | |
| S7211A; pH 7 | 47.76 | 9.53 | 0.74 | 4.05 | 1.37 |
| S7211B; pH 7 | 47.73 | 9.53 | 0.79 | 4.02 | 1.36 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 94
Unsaturated fatty acid profile in S3150, S7211 and
representative derivative transgenic lines transformed
with pSZ5350 (BrDAG-CPT at PLSC-2/LPAAT1-2 genomic
locus) DNA.

| | | | | Sum | |
|---|---|---|---|---|---|
| Sample ID | C18:1 | C18:2 | C18:3a | C20:1 | C22:1 |
| S7211; T1181; | 36.84 | 15.57 | 1.69 | 6.21 | 4.09 |
| D4211-20; pH 7 | | | | | |
| S7211; T1181; | 37.87 | 14.56 | 1.90 | 6.14 | 3.92 |
| D4211-8; pH 7 | | | | | |
| S7211; T1181; | 38.49 | 14.39 | 1.58 | 5.86 | 3.67 |
| D4211-18; pH 7 | | | | | |
| S7211; T1181; | 40.12 | 14.08 | 1.65 | 5.93 | 3.57 |
| D4211-2; pH 7 | | | | | |
| S7211; T1181; | 38.45 | 15.17 | 1.36 | 5.52 | 2.94 |
| D4211-3; pH 7 | | | | | |
| S7211; pH 7 | 47.81 | 10.21 | 0.88 | 4.27 | 1.54 |
| S7211; pH 7 | 47.96 | 10.11 | 0.90 | 4.28 | 1.55 |
| S3150; pH 7 | 57.99 | 6.62 | 0.56 | 0.19 | 0 |
| S3150; pH 5 | 57.7 | 7.08 | 0.54 | 0.11 | 0 |

TABLE 95
Unsaturated fatty acid profile in S3150, S7211 and
representative derivative transgenic lines transformed
with pSZ5306 (BjDAG-CPT at PLSC-2/LPAAT1-1 genomic
locus) DNA.

| | | | | Sum | |
|---|---|---|---|---|---|
| Sample ID | C18:1 | C18:2 | C18:3a | C20:1 | C22:1 |
| S7211; T1173; | 35.10 | 14.35 | 1.18 | 5.64 | 4.43 |
| D4167-4; pH 7 | | | | | |
| S7211; T1173; | 41.05 | 13.35 | 1.48 | 5.68 | 3.41 |
| D4167-1; pH 7 | | | | | |
| S7211; T1173; | 41.72 | 13.18 | 1.48 | 5.49 | 3.00 |
| D4167-7; pH 7 | | | | | |
| S7211; T1173; | 43.95 | 12.31 | 1.19 | 5.14 | 2.62 |
| D4167-5; pH 7 | | | | | |
| S7211; T1173; | 45.19 | 11.65 | 1.09 | 4.78 | 2.32 |
| D4167-10; pH 7 | | | | | |
| S7211A; pH 7 | 48.23 | 9.69 | 0.75 | 4.02 | 1.34 |
| S7211B; pH 7 | 48.24 | 9.65 | 0.75 | 4.01 | 1.33 |
| S3150; pH 7 | 57.99 | 6.62 | 0.56 | 0.19 | 0.00 |
| S3150; pH 5 | 57.70 | 7.08 | 0.54 | 0.11 | 0.00 |

TABLE 96
Unsaturated fatty acid profile in S3150, S7211 and
representative derivative transgenic lines transformed
with pSZ55347 (BjDAG-CPT at PLSC-2/LPAAT1-2 genomic
locus) DNA.

| | | | | Sum | |
|---|---|---|---|---|---|
| Sample ID | C18:1 | C18:2 | C18:3a | C20:1 | C22:1 |
| S7211; T1181; | 38.61 | 13.92 | 1.50 | 6.21 | 4.38 |
| D4208-11; pH 7 | | | | | |
| S7211; T1181; | 37.66 | 14.22 | 0.98 | 6.04 | 3.67 |
| D4208-15; pH 7 | | | | | |
| S7211; T1181; | 40.69 | 13.04 | 1.46 | 5.55 | 3.45 |
| D4208-5; pH 7 | | | | | |
| S7211; T1181; | 40.27 | 13.43 | 1.51 | 5.94 | 3.41 |
| D4208-10; pH 7 | | | | | |
| S7211; T1181; | 39.83 | 13.84 | 1.33 | 5.13 | 2.29 |
| D4208-20; pH 7 | | | | | |
| S7211; pH 7 | 47.81 | 10.21 | 0.88 | 4.27 | 1.54 |
| S7211; pH 7 | 47.96 | 10.11 | 0.90 | 4.28 | 1.55 |
| S3150; pH 7 | 57.99 | 6.62 | 0.56 | 0.19 | 0.00 |
| S3150; pH 5 | 57.70 | 7.08 | 0.54 | 0.11 | 0.00 |

### Example 16

### Expression of LPCAT in a High-Linolenic Transgenic Microalga

In this example we demonstrate using higher plant Lysophosphatidylcholine acyltransferase (LPCAT) genes to alter the content and composition of oils in transgenic algal strains for producing oils rich in linoleic and/or linolenic acids. A. thaliana LPCAT2 (AtLPCAT2 NP_176493.1) and B. rapa LPCAT (BrLPCAT) nucleic acid sequences were discussed herein in Examples 11 and 12. The sequences of both AtLPCAT1 and BrLPCAT were codon optimized for expression in our host and expressed in S3709. S3709 is described in Example 14. Our results show that expression of heterologous LPCAT enzymes S3709 more than doubles the C18:3 content in individual lines over the parents.

Construct Used for the Expression of the A. thaliana Lysophosphatidylcholine Acyltransferase-2 (AtLPCAT2) in Linolenic Strain S3709 [pSZ5297]:

In this example, transgenic lines from S3709, transformed with the construct pSZ5297, were generated which express Sacharomyces carlbergenesis MEL1 gene (allowing for their selection and growth on medium containing melibiose) and A. thaliana LPCAT2 (AtLPCAT2) gene targeted at endogenous PmLPAAT1-1 genomic region. Construct pSZ5297 introduced for expression in S3709 can be written as PLSC-2/LPAAT1-1 5′ flank::PmHXT1-ScarMEL1-CvNR:PmSAD2-2v2-AtLPCAT2-CvNR::PLSC-2/LPAAT1-1 3′ flank.

The sequence of the transforming DNA is provided below. Relevant restriction sites in the construct are indicated in lowercase, underlined bold, and are from 5′-3′ BspQI, KpnI, SpeI, SnaBI, EcoRI, SpeI, AflII, SacI, BspQI, respectively. BspQI sites delimit the 5′ and 3′ ends of the transforming DNA. Bold, lowercase sequences represent genomic DNA from S3150 that permit targeted integration at the PLSC-2/LPAAT1-1 locus via homologous recombination. Proceeding in the 5′ to 3′ direction, the endogenous P. moriformis Hexose Transporter 1 promoter driving the expression of the S. carlbergenesis MEL1 gene (encoding an alpha galactosidase enzyme activity required for catabolic conversion of Meliobise to glucose and galactose, thereby permitting the transformed strain to grow on melibiose) is indicated by lowercase, boxed text. The initiator ATG and terminator TGA for MEL1 are indicated by uppercase italics, while the coding region is indicated with lowercase italics. The Chlorella vulgaris nitrate reductase (NR) gene 3′ UTR is indicated by lowercase underlined text followed by an endogenous PMSAD2-v2 promoter of P. moriformis, indicated by boxed italicized text. The Initiator ATG and terminator TGA codons of the AtLPCAT2 are indicated by uppercase, bold italics, while the remainder of the gene is indicated by bold italics. The C. vulgaris nitrate reductase 3′ UTR is again indicated by lowercase underlined text followed by the S1920 PLSC-2/LPAAT1-1 genomic region indicated by bold, lowercase text. The final construct was sequenced to ensure correct reading frames and targeting sequences.

Nucleotide sequence of transforming DNA contained in plasmid pSZ5297:
(SEQ ID NO: 137)
gctcttctgcttcggattccactacatcaagtgggtgaacctggcgggcgcggaggagggcccccgcccgggcggcattgtta
gcaaccactgcagctacctggacatcctgctgcacatgtccgattccaccccgcctttgtggcgcgccagtcgacggccaagc
tgccctttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg
gccggggtgcccgtccagcccgtggtaccgcggtgagaatcgaaaatgcatcgtttctaggttcggagacggtcaattccctgctcc
ggcgaatctgtcggtcaagctggccagtggacaatgttgctatggcagcccgcgcacatgggcctcccgacgcggccatcaggagc
ccaaacagcgtgtcagggtatgtgaaactcaagaggtccctgctgggcactccggccccactccgggggcgggacgccaggcattc
gcggtcggtcccgcgcgacgagcgaaatgatgattcggttacgagaccaggacgtcgtcgaggtcgagaggcagcctcggacacg
tctcgctagggcaacgccccgagtccccgcgagggccgtaaacattgtttctgggtgtcggagtgggcattttgggcccgatccaatc
gcctcatgccgctctcgtctggtcctcacgttcgcgtacggcctggatcccggaaagggcggatgcacgtggtgttgccccgccattg
gcgcccacgtttcaaagtccccggccagaaatgcacaggaccggcccggctcgcacaggccatgctgaacgcccagatttcgaca
gcaacaccatctagaataatcgcaaccatccgcgttttgaacgaaacgaaacggcgctgtttagcatgtttccgacatcgtgggggccg
aagcatgctccggggggaggaaagcgtggcacagcggtagcccattctgtgccacacgccgacgaggaccaatccccggcatca

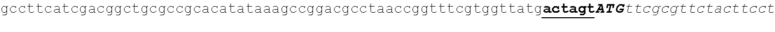

gacggcctgcatctccctgaagggcgtgttcggcgtctccccctcctacaacggcctgggcctgacgccccagatgggctgggac
aactggaacacgttcgcctgcgacgtctccgagcagctgctgctggacacggccgaccgcatctccgacctgggcctgaaggac
atgggctacaagtacatcatcctggacgactgctggtcctccggccgcgactccgacggcttcctggtcgccgacgagcagaagt
tccccaacggcatgggccacgtcgccgaccacctgcacaacaactccttcctgttcggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgtgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgttcggccagaagatcggctccctgtcccccaacgcgatcctgaacacg

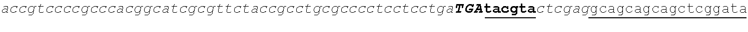

gtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtggg
atgggaacacaaatggaaagctgtagaattcctggctcgggcctcgtgctggcactccctcccatgccgacaacctttctgctgtcacc
acgacccacgatgcaacgcgacacgacccggtgggactgatcggttcactgcacctgcatgcaattgtcacaagcgcatactccaat
cgtatccgtttgatttctgtgaaaactcgctcgaccgcccgcgtcccgcaggcagcgatgacgtgtgcgtgacctgggtgtttcgtcga
aaggccagcaaccccaaatcgcaggcgatccggagattgggatctgatccgagcttggaccagatcccccacgatgcggcacggg
aactgcatcgactcggcgcggaacccagcatcgtaaatgccagattggtgtccgataccttgatttgccatcagcgaaacaagacttca
gcagcgagcgtataggcgggcgtgctaccagggagcatacattgcccatactgtctggaccgcataccggcgcagagggtgagtt
gatggggaggcaggcatcgaaacgcgcgtgcatggtgtgtgtgtctgattcggctgcacaatttcaatagtcggatgggcgacggta
gaattgggtgagcgctcgcgtgcatgcctcgccccgtcgggtgtcatgaccgggactggaatcccccctcgcgaccctcctgctaac

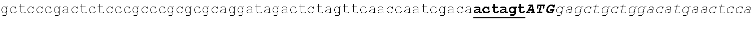

tggccgcctccatcggcgtgtccgtggccgtgctgcgcttcctgctgtgcttcgtggccaccatccccatctccttcctgtggcgcttca
tcccctcccgcctgggcaagcacatctactccgccgcctccggcgccttcctgtcctacctgtccttcggcttctcctccaacctgcac
ttcctggtgcccatgaccatcggctacgcctccatggccatctaccgccccctgtccggcttcatcaccttcttcctgggcttcgcctac
ctgatcggctgccacgtgttctacatgtccggcgacgcctggaaggagggcggcatcgactccaccggcgccctgatggtgctga
ccctgaaggtgatctcctgctccatcaactacaacgacggcatgctgaaggaggagggcctgcgcgaggcccagaagaagaa
ccgcctgatccagatgccctccctgatcgagtacttcggctactgcctgtgctgcggctcccacttcgccggccccgtgacgagatg
aaggactacctggagtggaccgaggagaagggcatctgggccgtgtccgagaagggcaagcgcccctccccctacggcgcca
tgatccgcgccgtgaccaggccgccatctgcatggccctgtacctgtacctggtgccccagttccccctgacccgcttcaccgagc
ccgtgtaccaggagtggggcttcctgaagcgcttcggctaccagtacatggccggcttcaccgcccgctggaagtactacttcatct
ggtccatctccgaggcctccatcatcatctccggcctgggcttctccggctggaccgacgagacccagaccaaggccaagtggg
accgcgccaagaacgtggacatcctgggcgtggagctggccaagtccgccgtgcagatccccctgttctggaacatccaggtgtc
cacctggctgcgccactacgtgtacgagcgcatcgtgaagcccggcaagaaggccggcttatccagctgctggccacccagac
cgtgtccgccgtgtggcacggcctgtaccccggctacatcatcttcttcgtgcagtccgccctgatgatcgacggctccaaggccat
ctaccgctggcagcaggccatcccccccaagatggccatgctgcgcaacgtgctggtgctgatcaacttcctgtacaccgtggtgg
tgctgaactactcctccgtgggcttcatggtgctgtccctgcacgagaccctggtggccttcaagtccgtgtactacatcggcaccgt

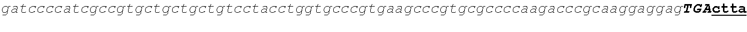

aggcagcagcagctcggatagtatcgacacactctggacgctggtcgtgtgatggactgagccgccacacttgctgccttgacctgtg
aatatccctgccgcattatcaaacagcctcagtgtgatgatcagtgtgtacgcgcattgcgagagctagctgcttgtgctatttgcgaat
accacccccagcatccccaccctcgatcatatcgcttgcatcccaaccgcaacttatctacgctgtcctgctatccctcagcgctgctcc
tgctcctgctcactgcccctcgcacagccttggtttgggctccgcctgtattctcctggtactgcaacctgtaaaccagcactgcaatgct
gatgcacgggaagtagtgggatgggaacacaaatggaaagcttaattaagagctccgtcctccactaccacagggtatggtcgtgt
ggggtcgagcgtgttgaagcgcagaaggggatgcgccgtcaagatcaggagctaaaaatggtgccagcgaggatccagcg
ctctcactcttgctgccatcgctcccacccttttccccaggggaccctgtggcccacgtgggagacgattccggccaagtggcac
atcttcctgatgctctgccacccccgccacaaagtgaccgtgatgaaggttaggacaagggtcgggacccgattctggatatg
acctctgaggtgtgtttctcgcgcaagcgtcccccaattcgttacaccacatccctcacaccctcgcccctgacactcgcagttg
cccgtgtacgtccccaatgaggaggaaaaggccgaccccaagctgtacgcccaaaacgtccgcaaagccatggtgcgtcgg
gaaccgtcaaagtttgcttgcgggtgggcggggcggctctagcgaattggctcattggccctcaccgaggcagcacatcggac
accagtcgccacccggcttgcatcttcgccccctttcttctcgcagatggaggtcgccgggaccaaggacacgacggcggtgtt
tgaggacaagatgcgctacctgaactccctgaagagaaagtacggcaagcctgtgcctaagaaaattgagtgaacccccgtc
gtcgaccagaagagc

Constructs Used for the Expression of the BrLPCAT in S3709:

In addition to the A. thaliana LPCAT2 targeted at PLSC-2/PmLPAAT1-1 locus (pSZ5297), B. rapa LPCAT targeted at PLSC-2/PmLPAAT1-1 locus (pSZ5299) was also constructed for expression in S3709. The construct can be described as:

pSZ5299 PLSC-2/LPAAT1-1::PmHXT1-ScarMEL1-CvNR:PmSAD2-2v2-BrLPCAT-CvNR::PLSC-2/LPAAT1-1

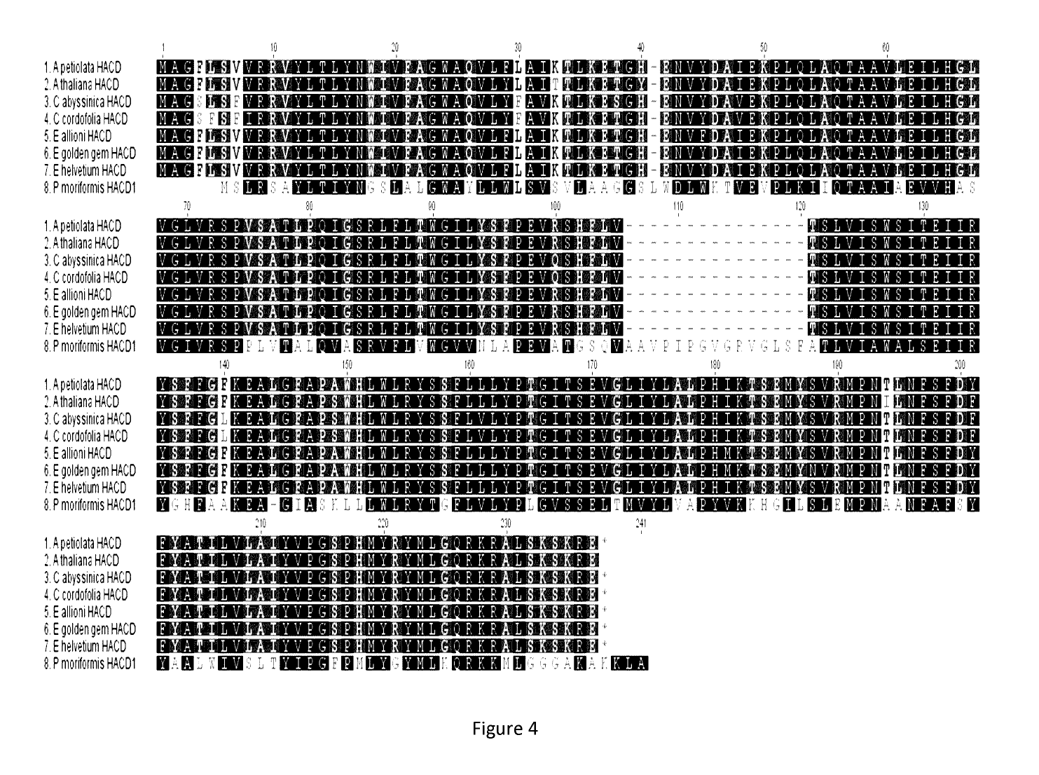

pSZ5299 has the same vector backbone; selectable marker, promoters, and 3′ utr as pSZ5297, differing only in the respective LPCAT gene. Relevant restriction sites in these constructs are also the same as in pSZ5296. FIGS. 5-4 indicate the sequence of PLSC-2/LPAAT1-2 5′ flank, PLSC-2/LPAAT1-2 3′ flank and AtLPCAT1, AtLPCAT2, BrLPCAT, BjLPCAT1, BjLPCAT2, LimdLPCAT1 and LimdLPCAT2 genes respectively. Relevant restriction sites as bold text are shown 5′-3′ respectively. The BrLPCAT sequence is shown below.

Nucleotide sequence of B. rapa LPCAT (BrLPCAT) contained in pSZ5299:
(SEQ ID NO: 138)

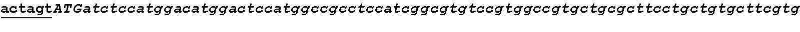

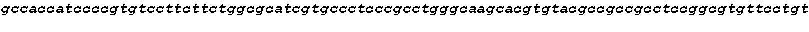

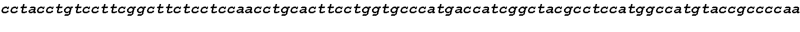

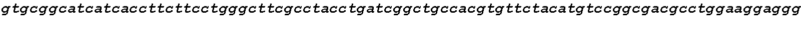

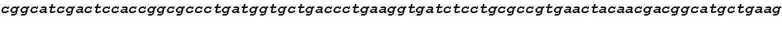

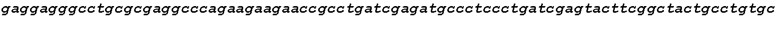

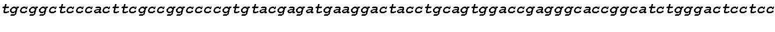

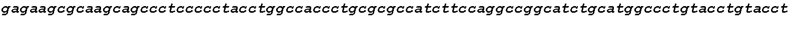

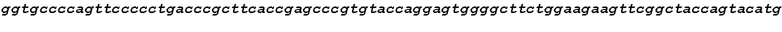

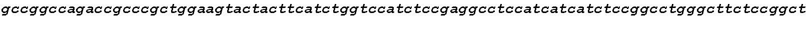

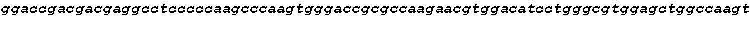

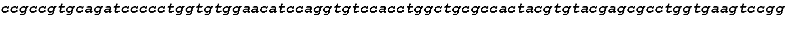

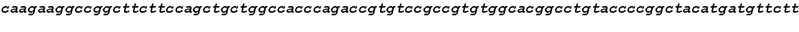

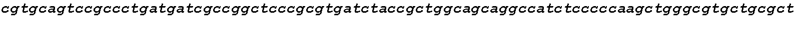

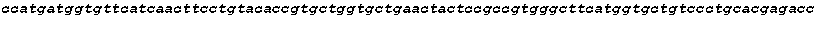

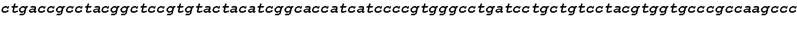

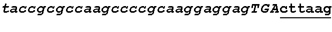

To determine their impact on fatty acid profiles, both constructs described above were transformed independently into S3709. Primary transformants were clonally purified and grown under standard lipid production conditions at pH7.0. The resulting fatty acid profiles from a set of representative clones arising from transformations with pSZ5297 (D4158) and pSZ5299 (D4160) into S3709 are shown in Tables 97 and 98, respectively.

All the transgenic lines expressing any of the above described LPCAT genes resulted in significant increase in C18:3. The increase in C18:3 in S3709; T1228; D4158-10; pH7 was 1.8 fold (12%) while the increase was 1.76 fold in S3709; T1228; D4160-17; pH7 (11.75%) over the parent S3709 (6.66%). However, unlike S3709 parent, not all of the available C18:2 was converted into C18:3 most likely due to sub-optimal expression of BnFAD3 in S3709. The conversion could be further enhanced by either optimizing the B. napus FAD3 activity in S3709 or expressing a better FAD3 enzyme activity from another higher plant like Flax.

TABLE 97
Unsaturated fatty acid profile in S3709 and representative derivative
transgenic lines transformed with pSZ5297 (AtLPCAT2 at PLSC-2/
LPAAT1-1 genomic locus) DNA.

| Sample ID | 14:0 | 16:0 | 18:0 | 18:1 | 18:2 | 18:3 a |
|---|---|---|---|---|---|---|
| S3709; pH 7 | .86 | 8.85 | .54 | 7.22 | .42 | .66 |
| S3709; pH 7 | .90 | 9.00 | .54 | 6.89 | .45 | .81 |
| S3709; T1228; D4158-10; | .12 | 1.92 | .97 | 6.70 | .78 | 2.00 |
| pH 7 | | | | | | |
| S3709; T1228; D4158-1; | .91 | 8.78 | .67 | 9.68 | .04 | 1.94 |
| pH 7 | | | | | | |
| S3709; T1228; D4158-19; | .21 | 8.62 | .05 | 6.28 | .46 | 1.47 |
| pH 7 | | | | | | |
| S3709; T1228; D4158-20; | .68 | 9.79 | .09 | 7.92 | .23 | 1.34 |
| pH 7 | | | | | | |
| S3709; T1228; D4158-11; | .63 | 0.32 | .10 | 7.74 | .19 | 0.95 |
| pH 7 | | | | | | |

TABLE 98
Unsaturated fatty acid profile in S3150, S7211 and representative
derivative transgenic lines transformed with pSZ5299 (BrLPCAT
at PLSC-2/LPAAT1-1 genomic locus) DNA.

| Sample ID | 14:0 | 16:0 | 18:0 | 18:1 | 18:2 | 18:3 a |
|---|---|---|---|---|---|---|
| S3709; pH 7 | .86 | 8.85 | .54 | 7.22 | .42 | .66 |
| S3709; pH 7 | .90 | 9.00 | .54 | 6.89 | .45 | .81 |
| S3709; T1228; D4160-17; | .98 | 9.37 | .74 | 9.80 | .19 | 1.75 |
| pH 7 | | | | | | |
| S3709; T1228; D4160-40; | .41 | 8.90 | .03 | 8.67 | .62 | 1.54 |
| pH 7 | | | | | | |
| S3709; T1228; D4160-26; | .64 | 9.94 | .11 | 8.14 | .88 | 1.53 |
| pH 7 | | | | | | |
| S3709; T1228; D4160-18; | .57 | 0.03 | .06 | 7.99 | .47 | 1.26 |
| pH 7 | | | | | | |
| S3709; T1228; D4160-4; | .03 | 1.42 | .92 | 7.43 | .95 | 0.89 |
| pH 7 | | | | | | |

The described embodiments of the invention are intended to be merely exemplary and numerous variations and modifications will be apparent to those skilled in the art. All such variations and modifications are intended to be within the scope of the present invention. For example, where a knockout of a gene is called for, an equivalent result may be reached using knockdown techniques including mutation and expression of inhibitory substances such as RNAi or antisense.

### Example 17

### Algal Strain and Oil with Less than 4% Saturated Fat, Less than 1% C18:2, and Greater than 90% C18:1

In this example, we describe strains where we have modified the fatty acid profile to maximize the accumulation of oleic acid, and minimize the total saturates and polyunsaturates, by down-regulating endogenous FATA or FAD2 activity, over-expression of KASII or SAD2 genes. The resulting strains, including S8695, produce oils with >94% C18:1, <4% total saturates, and <1% C18:2. S8696, a clonal isolate prepared in the same manner as S8695 had essentially identical fatty acid profiles.

The strain, S8695 was created by three successive transformations. The high oleic base strain S7505 was first transformed with pSZ4769 (FAD2 5′1-PmHXT1V2-ScarMEL1-PmPGK-PmSAD2-2p-PmKASII-CvNR-PmSAD2-2P-PmSAD2-1-CvNR-FAD2 3′), in which a construct that disrupts a single copy of the FAD2 allele while simultaneously overexpressing the P. moriformis KASII and PmSAD2-1. The resulting strain S8045 produces 87.3% C18:1 with total saturates 7.3%, under same condition; S7505 produces 18.9% total saturates (Table 99).

S8045 was subsequently transformed with pSZ5173 (FATA1 3′::CrTUB2-ScSUC2-CvNR:CrTUB2-HpFAD2-CvNR::FATA1 5′), a construct disrupts FATA allele1 to further reduce C16:0, and express a hairpin FAD2 to reduce C18:2. One of the resulting strains, S8197, produces 0.5% C18:2 and the total saturates level drop to 4.9%, due to the reduction of C16:0 fatty acid. We also observed that although S8197 is stable for sucrose invertase marker, the sucrose hydrolysis activity of this strain is less than ideal.

Strain S8197 was then transformed with pSZ5563 (6SA::PmLDH1-AtThic-PmHSP90: CrTUB2-ScSUC2-PmPGH-CvNR:PmSAD2-2V2-OeSAD-CvNR::6SB), a construct to over express one more stearoyl-ACP desaturase gene from Olea europaea. Goal of this transformation is to further reduce total saturates level. To increase sucrose hydrolysis activity in strain S8197, we also introduced an additional copy of sucrose invertase gene in pSZ5563. The resulting strain S8695 produces 1.6% C18:0, as oppose to 2.1% in S8197, therefore, the saturates level in S8695 is around 0.5% less than its parental strain S8197.

TABLE 99
Comparison of fatty acid profiles between strains S7505,
S8045, S8197 and S8695 in shake-flask experiment.

| | Fatty Acids Area % | | | | |
|---|---|---|---|---|---|
| Strains | C16:0 | C18:0 | C18:1 | C18:2 | Total saturates % |
| S7505 | 12.5 | 5.6 | 75.5 | 4.8 | 18.9 |
| S8045 | 4.3 | 2.1 | 87.3 | 3.9 | 7.3 |
| S8197 | 2.3 | 2.1 | 92.3 | 0.6 | 4.9 |
| S8695 | 2.4 | 1.6 | 92.7 | 0.5 | 4.5 |
| S8695 | 1.5 | 1.5 | 94.1 | 0.4 | 3.6 |

Generation of Strain S8045:

Strain S8045 is one of the transformants generated from pSZ4769 (FAD2 5′1-PmHXT1V2-ScarMEL1-PmPGK-PmSAD2-2p-PmKASII-CvNR-PmSAD2-2P-PmSAD2-1-CvNR-FAD2 3′) transforming high oleic base strain S7505. The sequence of the pSZ4769 transforming DNA is provided below. Relevant restriction sites in the construct are indicated in lowercase, bold and underlining and are 5′-3′ BspQ 1, Kpn I, Spe I, SnaBI, BamHI, AvrII, SpeI, ClaI, BamHI, SpeI, ClaI, Pad, BspQ I, respectively. BspQI sites delimit the 5′ and 3′ ends of the transforming DNA. Bold, lowercase sequences represent FAD2-1 5′ genomic DNA that permit targeted integration at Fad2-1 locus via homologous recombination. Proceeding in the 5′ to 3′ direction, the P. moriformis HXT1 promoter driving the expression of the Saccharomyces carlbergensis MEL1 gene is indicated by boxed text. The initiator ATG and terminator TGA for MEL1 gene are indicated by uppercase, bold italics while the coding region is indicated in lowercase italics. The P. moriformis PGK 3′ UTR is indicated by lowercase underlined text followed by the P. moriformis SAD2-2 promoter, indicated by boxed italics text. The Initiator ATG and terminator TGA codons of the PmKASII are indicated by uppercase, bold italics, while the remainder of the coding region is indicated by bold italics. The Chlorella protothecoides S106 stearoyl-ACP desaturase transit peptide is located between initiator ATG and the Asc I site. The Chlorella vulgaris nitrate reductase 3′ UTR is indicated by lowercase underlined text followed by another P. moriformis SAD2-2 promoter, indicated by boxed italics text. The Initiator ATG and terminator TGA codons of the PmSAD2-1 are indicated by uppercase, bold italics, while the remainder of the coding region is indicated by bold italics. The C. vulgaris nitrate reductase 3′ UTR is indicated by lowercase underlined text followed by the FAD2-1 3′ genomic region indicated by bold, lowercase text.

Nucleotide sequence of transforming DNA contained in pSZ4769:
(SEQ ID NO: 139)
gctcttcgcgaaggtcattttccagaacaacgaccatggcttgtcttagcgatcgctcgaatgactgctagtgagtcgtacgctcgacccagt
cgctcgcaggagaacgcggcaactgccgagcttcggcttgccagtcgtgactcgtatgtgatcaggaatcattggcattggtagcattata
attcggcttccgcgctgtttatgggcatggcaatgtctcatgcagtcgaccttagtcaaccaattctgggtggccagctccgggcgaccggg
ctccgtgtcgccgggcaccacctcctgccatgagtaacagggccgccctctcctcccgacgttggcccactgaataccgtgtcttggggccc
tacatgatgggctgcctagtcgggcgggacgcgcaactgcccgcgcaatctgggacgtggtctgaatcctccaggcgggtttccccgaga
aagaaagggtgccgatttcaaagcagagccatgtgccgggccctgtggcctgtgttggcgcctatgtagtcaccccccctcacccaattgtc
gccagtttgcgcaatccataaactcaaaactgcagcttctgagctgcgctgttcaagaacacctctggggtttgctcacccgcgaggtcgac

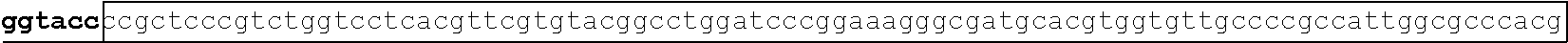

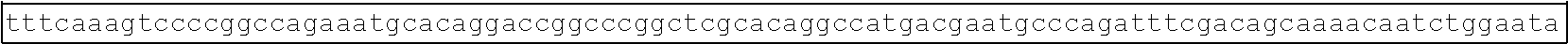

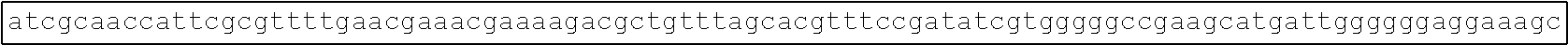

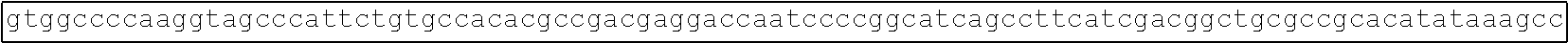

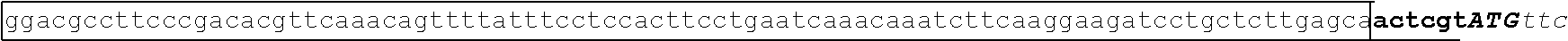

gcgttctacttcctgacggcctgcatctccctgaagggcgtgttcggcgtctccccctcctacaacggcctgggcctgacgccccagatgggctg
ggacaactggaacacgttcgcctgcgacgtctccgagcagctgctgctggacacggccgaccgcatctccgacctgggcctgaaggacatg
ggctacaagtacatcatcctggacgactgctggtcctccggccgcgactccgacggatcctggtcgccgacgagcagaagttccccaacggc
atgggccacgtcgccgaccacctgcacaacaactccttcctgttcggcatgtactcctccgcgggcgagtacacgtgcgccggctaccccggc
tccctgggccgcgaggaggaggacgcccagttcttcgcgaacaaccgcgtggactacctgaagtacgacaactgctacaacaagggccagt
tcggcacgcccgagatctcctaccaccgctacaaggccatgtccgacgccctgaacaagacgggccgccccatcuctactccctgtgcaact
ggggccaggacctgaccuctactggggctccggcatcgcgaactcctggcgcatgtccggcgacgtcacggcggagttcacgcgccccgac
tcccgctgcccctgcgacggcgacgagtacgactgcaagtacgccggatccactgctccatcatgaacatcctgaacaaggccgcccccat
gggccagaacgcgggcgtcggcggctggaacgacctggacaacctggaggtcggcgtcggcaacctgacggacgacgaggagaaggc
gcacttctccatgtgggccatggtgaagtcccccctgatcatcggcgcgaacgtgaacaacctgaaggcctcctcctactccatctactcccag
gcgtccgtcatcgccatcaaccaggactccaacggcatccccgccacgcgcgtctggcgctactacgtgtccgacacggacgagtacggcca
gggcgagatccagatgtggtccggccccctggacaacggcgaccaggtcgtggcgctgctgaacggcggctccgtgtcccgccccatgaac
acgaccctggaggagatcttcttcgactccaacctgggctccaagaagctgacctccacctgggacatctacgacctgtgggcgaaccgcgtc
gacaactccacggcgtccgccatcctgggccgcaacaagaccgccaccggcatcctgtacaacgccaccgagcagtcctacaaggacggc
ctgtccaagaacgacacccgcctgttcggccagaagatcggctccctgtcccccaacgcgatcctgaacacgaccgtccccgcccacggcat

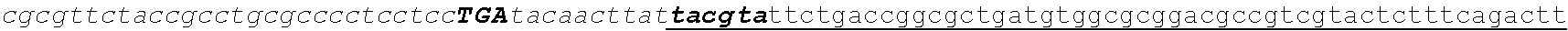

tactcttgaggaattgaacctttctcgcttgctggcatgtaaacattggcgcaattaattgtgtgatgaagaaagggtggcacaagatggatcgcgaat
gtacgagatcgacaacgatggtgattgttatgaggggccaaacctggctcaatcttgtcgcatgtccggcgcaatgtgatccagcggcgtgactctc
gcaacctggtagtgtgtgcgcaccgggtcgctttgattaaaactgatcgcattgccatcccgtcaactcacaagcctactctagctcccattgcgcact
cgggcgcccggctcgatcaatgttctgagcggagggcgaagcgtcaggaaatcgtctcggcagctggaagcgcatggaatgcggagcggagat

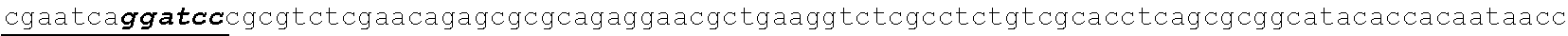

acctgacgaatgcgcttggttcttcgtccattagcgaagcgtccggttcacacacgtgcaacgttggcgaggtggcaggtgacaatgatcggtgga

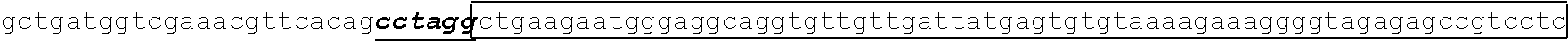

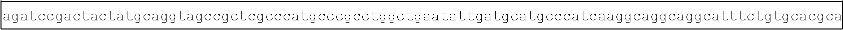

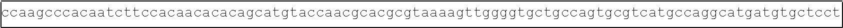

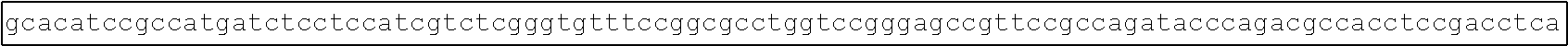

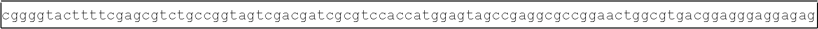

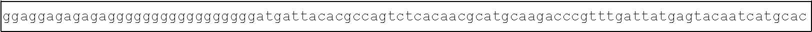

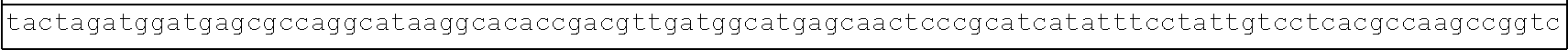

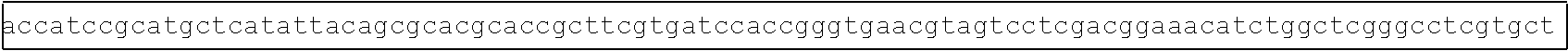

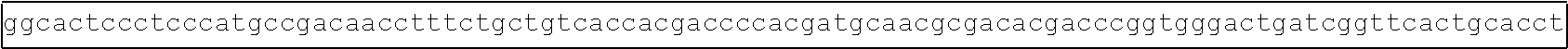

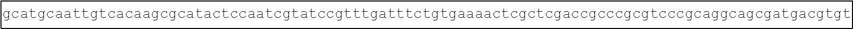

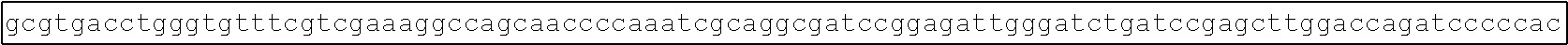

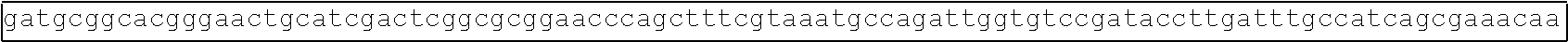

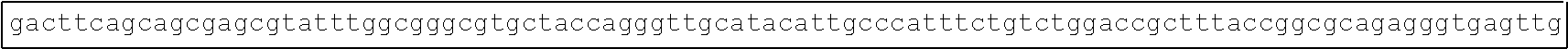

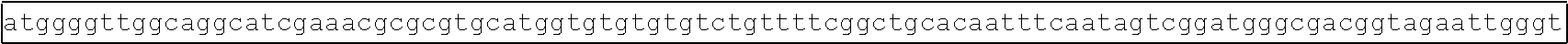

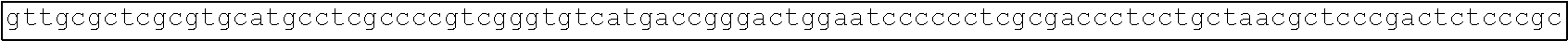

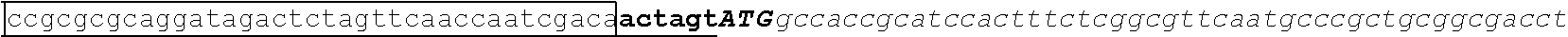

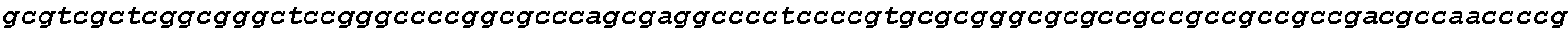

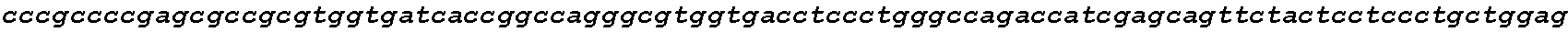

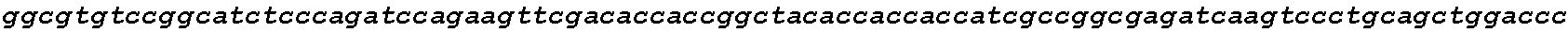

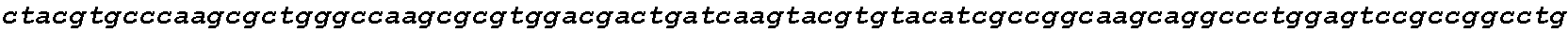

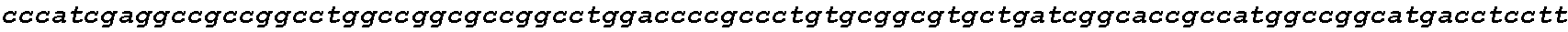

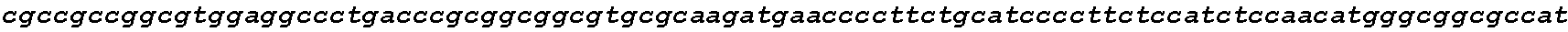

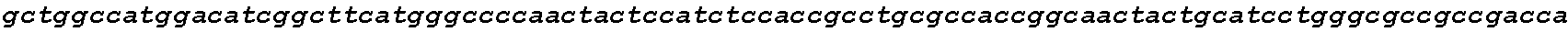

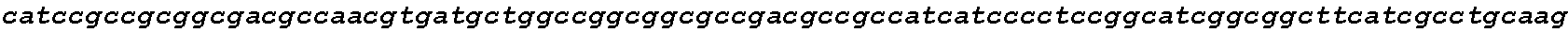

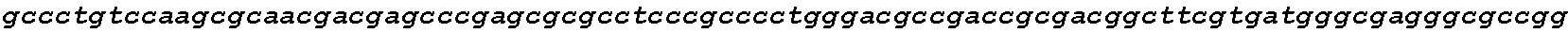

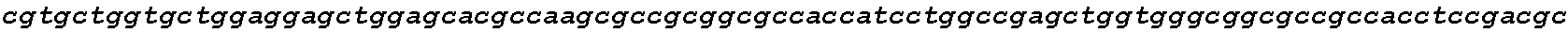

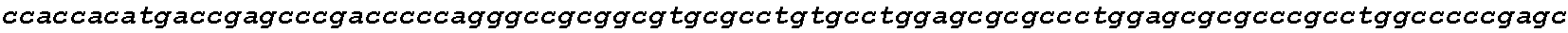

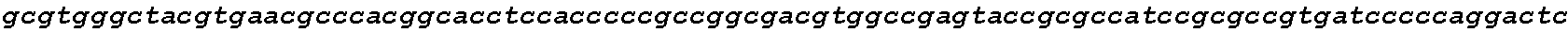

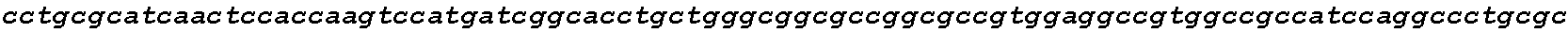

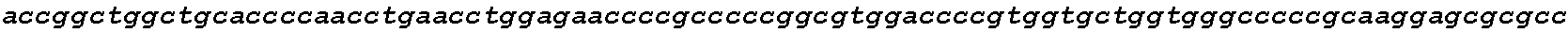

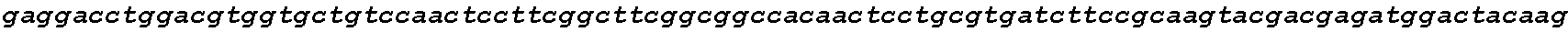

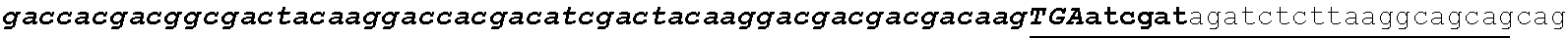

ctcggatagtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttccctcgtttc
atatcgcttgcatcccaaccgcaacttatctacgctgtcctgctatccctcagcgctgctcctgctcctgctcactgcccctcgcacagccttggtttgg
gctccgcctgtattctcctggtactgcaacctgtaaaccagcactgcaatgctgatgcacgggaagtagtgggatgggaacacaaatggagaattc

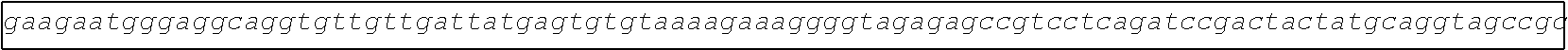

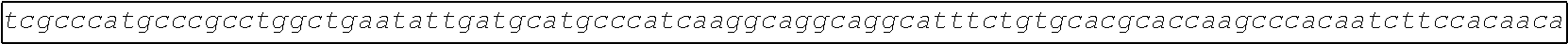

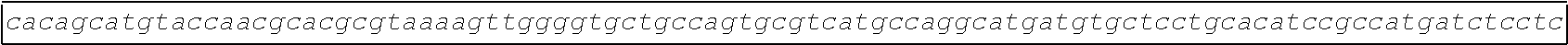

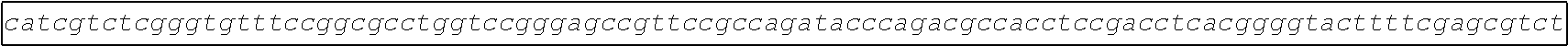

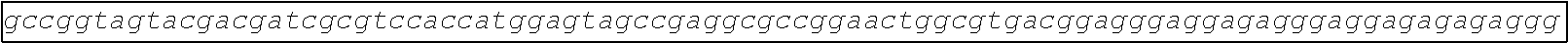

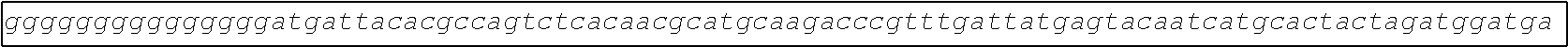

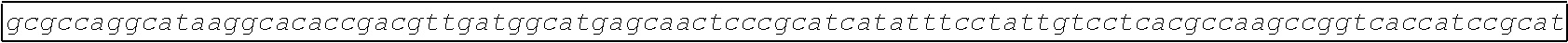

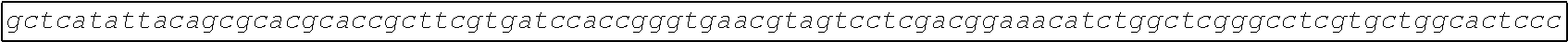

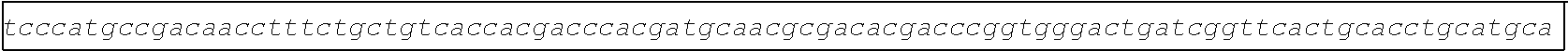

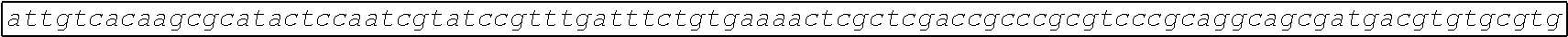

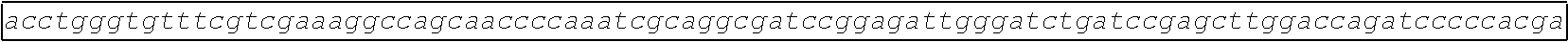

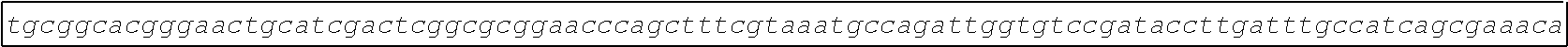

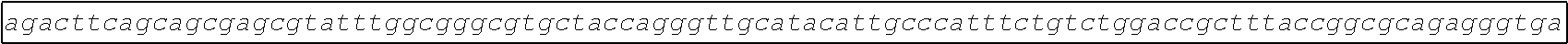

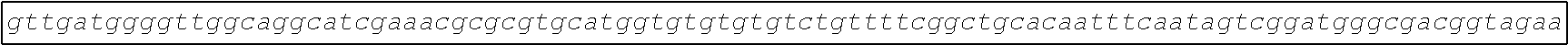

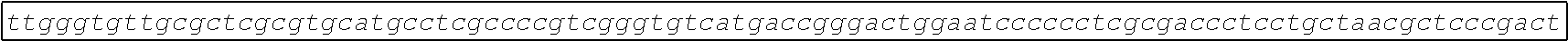

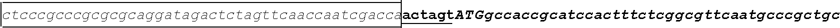

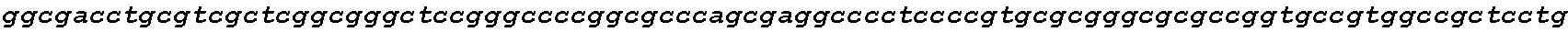

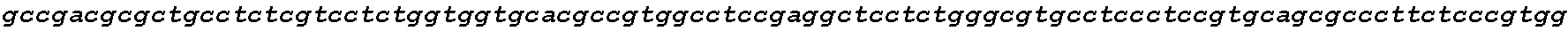

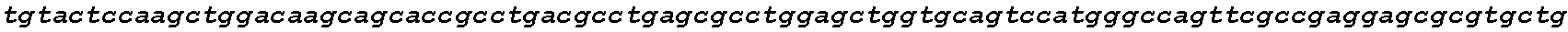

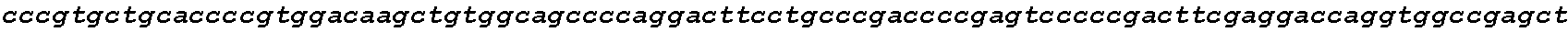

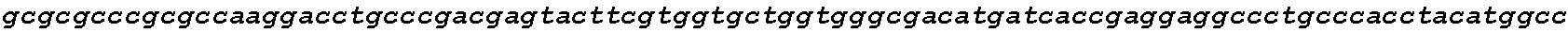

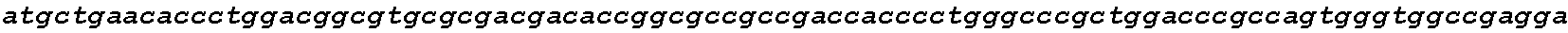

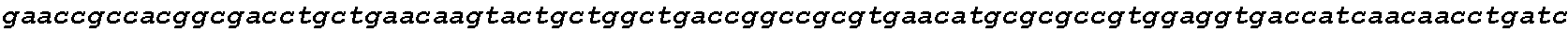

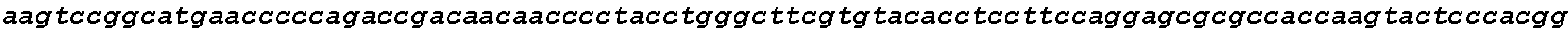

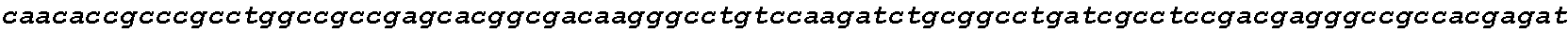

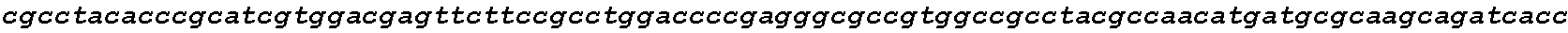

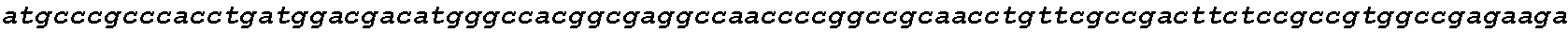

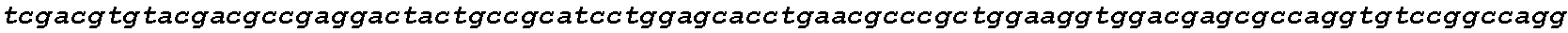

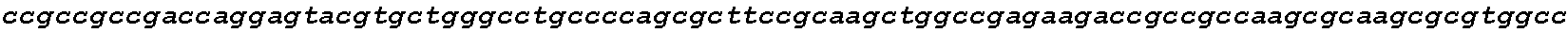

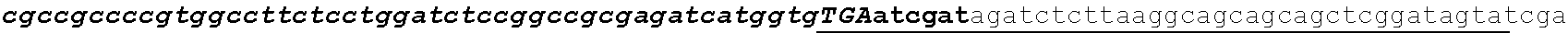

cacactctggacgctggtcgtgtgatggactgttgccgccacacttgctgccttgacctgtgaatatccctgccgcttttatcaaacagcctcagtgtgt
ttgatcttgtgtgtacgcgcttttgcgagttgctagctgcttgtgctatttgcgaataccacccccagcatcccccttccctcgtttcatatcgcttgcatccc
aaccgcaacttatctacgctgtcctgctatccctcagcgctgctcctgctcctgctcactgcccctcgcacagccttggtttgggctccgcctgtattct
cctggtactgcaacctgtaaaccagcactgcaatgctgatgcacgggaagtagtgggatgggaacacaaatggaaagcttaattaagagctcctc
actcagcgcgcctgcgcggggatgcggaacgccgccgccgccttgtcttttgcacgcgcgactccgtcgcttcgcgggtggcacccccatt
gaaaaaaacctcaattctgtttgtggaagacacggtgtacccccaaccacccacctgcacctctattattggtattattgacgcgggagcgg
gcgttgtactctacaacgtagcgtctctggttttcagctggctcccaccattgtaaattcttgctaaaatagtgcgtggttatgtgagaggtat
ggtgtaacagggcgtcagtcatgttggttttcgtgctgatctcgggcacaaggcgtcgtcgacgtgacgtgcccgtgatgagagcaatacc
gcgctcaaagccgacgcatggcctttactccgcactccaaacgactgtcgctcgtatttttcggatatctattttttaagagcgagcacagcg
ccgggcatgggcctgaaaggcctcgcggccgtgctcgtggtgggggccgcgagcgcgtggggcatcgcggcagtgcaccaggcgcaga
cggaggaacgcatggtgagtgcgcatcacaagatgcatgtcttgttgtctgtactataatgctagagcatcaccaggggcttagtcatcgca
cctgctttggtcattacagaaattgcacaagggcgtcctccgggatgaggagatgtaccagctcaagctggagcggcttcgagccaagca
ggagcgcggcgcatgacgacctacccacatgcgaagagc

Generation of Strain S8197:

Strain S8197 is one of the transformants generated from pSZ5173 (FATA1 3′::CrTUB2-ScSUC2-CvNR:CrTUB2-HpFAD2-CvNR::FATA1 5′) transforming strain S8045. The sequence of the pSZ5173 transforming DNA is provided below. Relevant restriction sites in the construct are indicated in lowercase, bold and underlining and are 5′-3′ BspQ I, Kpn I, AscI, MfeI, SpeI, SacI, BspQ I, respectively. BspQI sites delimit the 5′ and 3′ ends of the transforming DNA. Bold, lowercase sequences represent FATA1 3′ genomic DNA that permit targeted integration at FATA1 locus via homologous recombination.

Proceeding in the 5′ to 3′ direction, the C. reinhardtii β-tubulin promoter driving the expression of the yeast sucrose invertase gene is indicated by boxed text. The initiator ATG and terminator TGA for invertase are indicated by uppercase, bold italics while the coding region is indicated in lowercase italics. The C. vulgaris nitrate reductase 3′ UTR is indicated by lowercase underlined text followed by another C. reinhardtii β-tubulin promoter, indicated by boxed italics text. The hairpin FAD2 cassette is indicated by bold italics. The C. vulgaris nitrate reductase 3′ UTR is indicated by lowercase underlined text followed by the FATA1 5′ genomic region indicated by bold, lowercase text.

Nucleotide sequence of transforming DNA contained in pSZ5173:
(SEQ ID NO: 140)
gctcttcacccaactcagataataccaatacccctccttctcctcctcatccattcagtacccccccccttctcttcccaaagcagcaagcgcg
tggcttacagaagaacaatcggcttccgccaaagtcgccgagcactgcccgacggcggcgcgcccagcagcccgcttggccacacaggc
aacgaatacattcaatagggggcctcgcagaatggaaggagcggtaaagggtacaggagcactgcgcacaaggggcctgtgcaggag
tgactgactgggcgggcagacggcgcaccgcgggcgcaggcaagcagggaagattgaagcggcagggaggaggatgctgattgagg
ggggcatcgcagtctctcttggacccgggataaggaagcaaatattcggccggttgggttgtgtgtgtgcacgttttcttcttcagagtcgtg

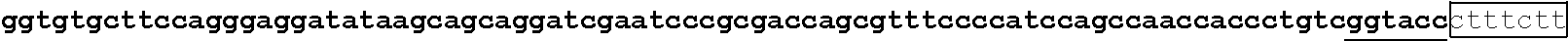

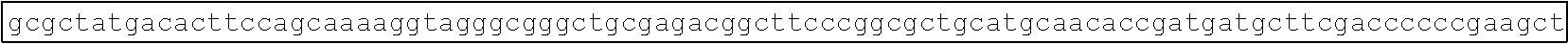

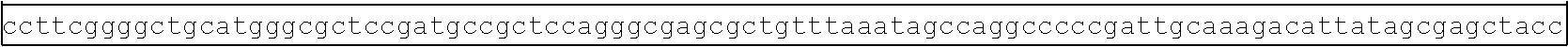

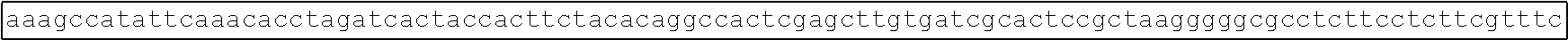

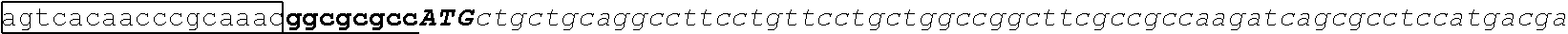

acgagacgtccgaccgccccctggtgcatcttcacccccaacaagggctggatgaacgaccccaacggcctgtggtacgacgagaaggacg
ccaagtggcacctgtacttccagtacaacccgaacgacaccgtctgggggacgcccttgttctggggccacgccacgtccgacgacctgacc
aactgggaggaccagcccatcgccatcgccccgaagcgcaacgactccggcgccttctccggctccatggtggtggactacaacaacacct
ccggctcttcaacgacaccatcgacccgcgccagcgctgcgtggccatctggacctacaacaccccggagtccgaggagcagtacatctcc
tacagcctggacggcggctacaccttcaccgagtaccagaagaaccccgtgctggccgccaactccacccagttccgcgacccgaaggtctt
ctggtacgagccctcccagaagtggatcatgaccgcggccaagtcccaggactacaagatcgagatctactcctccgacgacctgaagtcct
ggaagctggagtccgcgttcgccaacgagggcttcctcggctaccagtacgagtgccccggcctgatcgaggtccccaccgagcaggaccc
cagcaagtcctactgggtgatgttcatctccatcaaccccggcgccccggccggcggctcctcaaccagtacttcgtcggcagcttcaacggc
acccacttcgaggccttcgacaaccagtcccgcgtggtggacttcggcaaggactactacgccctgcagaccttcttcaacaccgacccgacc
tacgggagcgccctgggcatcgcgtgggcctccaactgggagtactccgccttcgtgcccaccaacccctggcgctcctccatgtccctcgtgc
gcaagttctccctcaacaccgagtaccaggccaacccggagacggagctgatcaacctgaaggccgagcgatcctgaacatcagcaacg
ccggcccctggagccggttcgccaccaacaccacgttgacgaaggccaacagctacaacgtcgacctgtccaacagcaccggcaccctgg
agttcgagctggtgtacgccgtcaacaccacccagacgatctccaagtccgtgttcgcggacctctccctctggttcaagggcctggaggaccc
cgaggagtacctccgcatgggcttcgaggtgtccgcgtcctccttcttcctggaccgcgggaacagcaaggtgaagttcgtgaaggagaaccc
ctacttcaccaaccgcatgagcgtgaacaaccagcccttcaagagcgagaacgacctgtcctactacaaggtgtacggcttgctggaccaga
acatcctggagctgtacttcaacgacggcgacgtcgtgtccacaccaacacctacttcatgaccaccgggaacgccctgggctccgtgaacatga

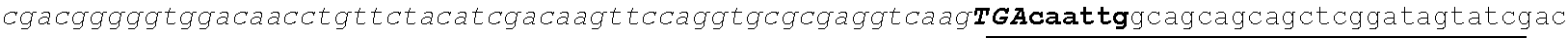

acactctggacgctggtcgtgtgatggactgttgccgccacacttgctgccttgacctgtgaatatccctgccgcttttatcaaacagcctcagtgtgttt
gatcttgtgtgtacgcgcttttgcgagttgctagctgcttgtgctatttgcgaataccacccccagcatccccttccctcgtttcatatcgcttgcatccca
accgcaacttatctacgctgtcctgctatccctcagcgctgctcctgctcctgctcactgcccctcgcacagccttggtttgggctccgcctgtattctc
ctggtactgcaacctgtaaaccagcactgcaatgctgatgcacgggaagtagtgggatgggaacacaaatggaggatcccgcgtctcgaacaga
gcgcgcagaggaacgctgaaggtctcgcctctgtcgcacctcagcgcggcatacaccacaataaccacctgacgaatgcgcttggttcttcgtcca
ttagcgaagcgtccggttcacacacgtgccacgttggcgaggtggcaggtgacaatgatcggtggagctgatggtcgaaacgttcacagcctagg

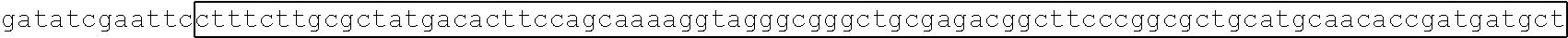

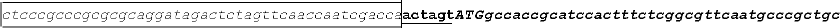

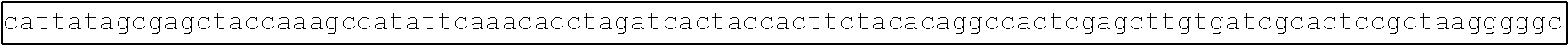

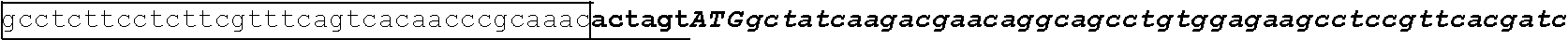

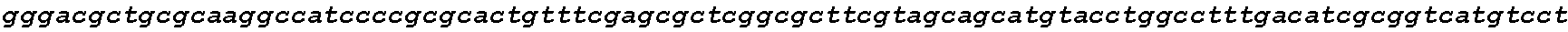

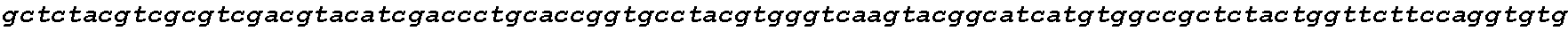

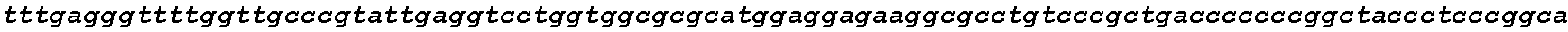

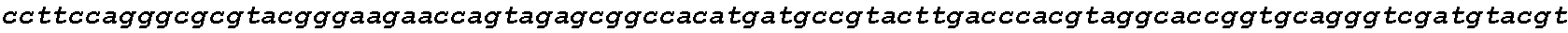

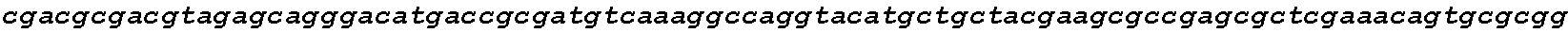

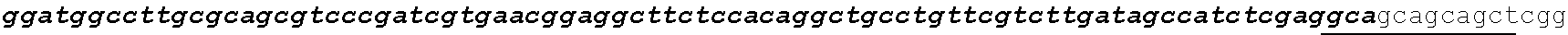

atagtatcgacacactctggacgctggtcgtgtgatggactgttgccgccacacttgctgccttgacctgtgaatatccctgccgcttttatcaaacagc
ctcagtgtgtttgatcttgtgtgtacgcgcttttgcgagttgctagctgcttgtgctatttgcgaataccacccccagcatccccttccctcgtttcatatcg
cttgcatcccaaccgcaacttatctacgctgtcctgctatccctcagcgctgctcctgctcctgctcactgcccctcgcacagccttggtttgggctccg
cctgtattctcctggtactgcaacctgtaaaccagcactgcaatgctgatgcacgggaagtagtgggatgggaacacaaatggaaagctgtagagc
tcttgttttccagaaggagttgctccttgagcctttcattctcagcctcgataacctccaaagccgctctaattgtggagggggacgaaccgaatgctg
cgtgaacgggaaggaggaggagaaagagtgagcagggagggattcagaaatgagaaatgagaggtgaaggaacgcatccctatgcc
cttgcaatggacagtgtttctggccaccgccaccaagacttcgtgtcctctgatcatcatgcgattgattacgttgaatgcgacggccggtca
gccccggacctccacgcaccggtgctcctccaggaagatgcgcttgtcctccgccatcttgcagggctcaagctgctcccaaaactcttggg
cgggttccggacggacggctaccgcgggtgcggccctgaccgccactgttcggaagcagcggcgctgcatgggcagcggccgctgcggt
gcgccacggaccgcatgatccaccggaaaagcgcacgcgctggagcgcgcagaggaccacagagaagcggaagagacgccagtact
ggcaagcaggctggtcggtgccatggcgcgctactaccctcgctatgactcgggtcctcggccggctggcggtgctgacaattcgtttagtg
gagcagcgactccattcagctaccagtcgaactcagtggcacagtgactccgctcttc

Generation of Strain S8695:

Strain S8695 is one of the transformants generated from pSZ5563 (6SA::PmLDH1-AtThic-PmHSP90: CrTUB2-ScSUC2-PmPGH-CvNR:PmSAD2-2V2-OeSAD-CvNR::6SB) transforming strain S8197. The sequence of the pSZ5563 transforming DNA is provided below. Relevant restriction sites in the construct are indicated in lowercase, bold and underlining and are 5′-3′ BspQ I, SpeI, KpnI, AscI, MfeI, AvrII, EcoRV, SpeI, AscI, ClaI, SacI, BspQ I, respectively. BspQI sites delimit the 5′ and 3′ ends of the transforming DNA. Bold, lowercase sequences represent 6SA genomic DNA that permits targeted integration at 6S locus via homologous recombination. Proceeding in the 5′ to 3′ direction, the P. moriformis LDH1 promoter driving the expression of the Arabidopsis thaliana THIC gene is indicated by boxed text. The initiator ATG and terminator TGA for THIC gene are indicated by uppercase, bold italics while the coding region is indicated in lowercase italics. The P. moriformis HSP90 3′ UTR is indicated by lowercase underlined text followed by C. reinhardtii β-tubulin promoter, indicated by boxed italics text. The initiator ATG and terminator TGA for invertase are indicated by uppercase, bold italics while the coding region is indicated in lowercase italics. The P. moriformis PGH 3′ UTR is indicated by lowercase underlined text followed by a C. vulgaris nitrate reductase 3′ UTR, indicated by lowercase underlined text. The P. moriformis SAD2-2 promoter, indicated by boxed italics text, is utilized to drive the expression of O. europaea SAD gene. The Initiator ATG and terminator TGA codons of the OeSAD are indicated by uppercase, bold italics, while the remainder of the coding region is indicated by bold italics. The C. protothecoides S106 stearoyl-ACP desaturase transit peptide is located between initiator ATG and the Asc I site. The C. vulgaris nitrate reductase 3′ UTR is indicated by lowercase underlined text followed by the 6SB genomic region indicated by bold, lowercase text.

Nucleotide sequence of transforming DNA contained in pSZ5563:
(SEQ ID NO: 141)
gctcttcgccgccgccactcctgctcgagcgcgcccgcgcgtgcgccgccagcgccttggccttttcgccgcgctcgtgcgcgtcgctgatgt
ccatcaccaggtccatgaggtctgccttgcgccggctgagccactgcttcgtccgggcggccaagaggagcatgagggaggactcctggt
ccagggtcctgacgtggtcgcggctctgggagcgggccagcatcatctggctctgccgcaccgaggccgcctccaactggtcctccagca
gccgcagtcgccgccgaccctggcagaggaagacaggtgaggggggtatgaattgtacagaacaaccacgagccttgtctaggcagaa
tccctaccagtcatggctttacctggatgacggcctgcgaacagctgtccagcgaccctcgctgccgccgcttctcccgcacgcttctttcca
gcaccgtgatggcgcgagccagcgccgcacgctggcgctgcgcttcgccgatctgaggacagtcggggaactctgatcagtctaaacccc

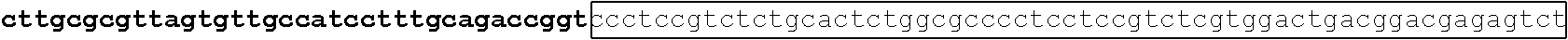

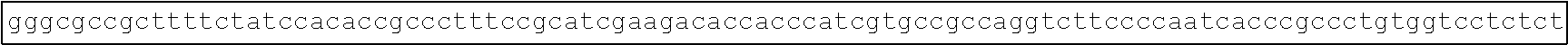

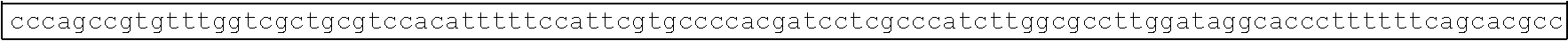

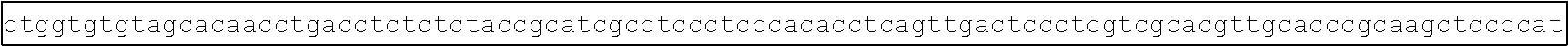

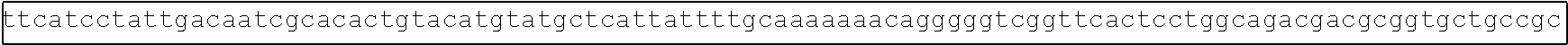

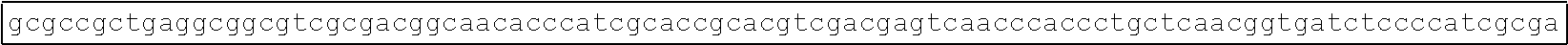

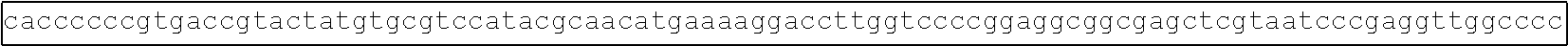

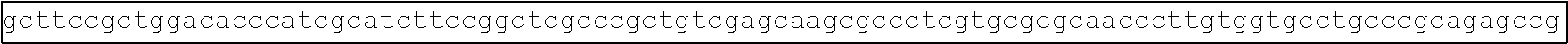

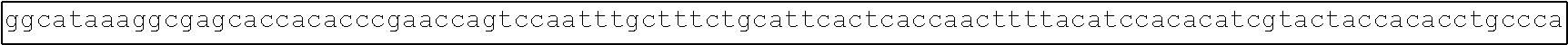

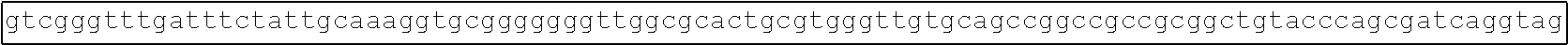

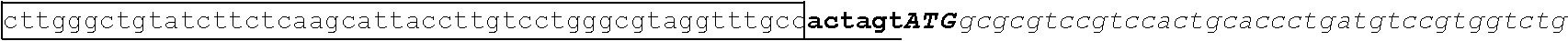

caacaacaagaaccactccgcccgccccaagctgcccaactcctccctgctgcccggcttcgacgtggtggtccaggccgcggccacccgct
tcaagaaggagacgacgaccacccgcgccacgctgacgttcgacccccccacgaccaactccgagcgcgccaagcagcgcaagcacac
catcgacccctcctcccccgacttccagcccatcccctccttcgaggagtgcttccccaagtccacgaaggagcacaaggaggtggtgcacga
ggagtccggccacgtcctgaaggtgcccttccgccgcgtgcacctgtccggcggcgagcccgccttcgacaactacgacacgtccggccccc
agaacgtcaacgcccacatcggcctggcgaagctgcgcaaggagtggatcgaccgccgcgagaagctgggcacgccccgctacacgcag
atgtactacgcgaagcagggcatcatcacggaggagatgctgtactgcgcgacgcgcgagaagctggaccccgagttcgtccgctccgagg
tcgcgcggggccgcgccatcatcccctccaacaagaagcacctggagctggagcccatgatcgtgggccgcaagttcctggtgaaggtgaa
cgcgaacatcggcaactccgccgtggcctcctccatcgaggaggaggtctacaaggtgcagtgggccaccatgtggggcgccgacaccatc
atggacctgtccacgggccgccacatccacgagacgcgcgagtggatcctgcgcaactccgcggtccccgtgggcaccgtccccatctacca
ggcgctggagaaggtggacggcatcgcggagaacctgaactgggaggtgttccgcgagacgctgatcgagcaggccgagcagggcgtgg
actacttcacgatccacgcgggcgtgctgctgcgctacatccccctgaccgccaagcgcctgacgggcatcgtgtcccgcggcggctccatcc
acgcgaagtggtgcctggcctaccacaaggagaacttcgcctacgagcactgggacgacatcctggacatctgcaaccagtacgacgtcgc
cctgtccatcggcgacggcctgcgccccggctccatctacgacgccaacgacacggcccagttcgccgagctgctgacccagggcgagctg
acgcgccgcgcgtgggagaaggacgtgcaggtgatgaacgagggccccggccacgtgcccatgcacaagatccccgagaacatgcaga
agcagctggagtggtgcaacgaggcgcccttctacaccctgggccccctgacgaccgacatcgcgcccggctacgaccacatcacctccgc
catcggcgcggccaacatcggcgccctgggcaccgccctgctgtgctacgtgacgcccaaggagcacctgggcctgcccaaccgcgacga
cgtgaaggcgggcgtcatcgcctacaagatcgccgcccacgcggccgacctggccaagcagcacccccacgcccaggcgtgggacgacg
cgctgtccaaggcgcgcttcgagttccgctggatggaccagttcgcgctgtccctggaccccatgacggcgatgtccttccacgacgagacgct
gcccgcggacggcgcgaaggtcgcccacttctgctccatgtgcggccccaagttctgctccatgaagatcacggaggacatccgcaagtacg
ccgaggagaacggctacggctccgccgaggaggccatccgccagggcatggacgccatgtccgaggagttcaacatcgccaagaagacg

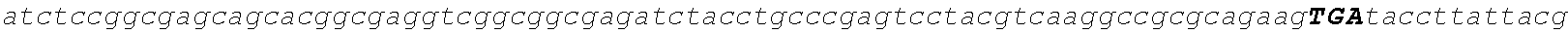

taacagacgaccttggcaggcgtcgggtagggaggtggtggtgatggcgtctcgatgccatcgcacgcatccaacgaccgtatacgcatcgtcca
atgaccgtcggtgtcctctctgcctccgttttgtgagatgtctcaggcttggtgcatcctcgggtggccagccacgttgcgcgtcgtgctgcttgcctct
cttgcgcctctgtggtactggaaaatatcatcgaggcccgtttttttgctcccatttcctttccgctacatcttgaaagcaaacgacaaacgaagcagca
agcaaagagcacgaggacggtgaacaagtctgtcacctgtatacatctatttccccgcgggtgcacctactctctctcctgccccggcagagtcagc

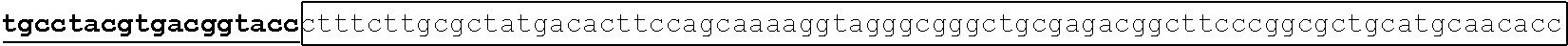

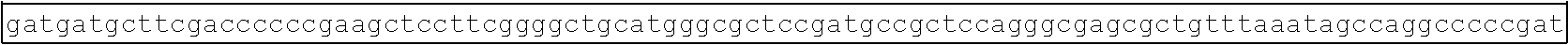

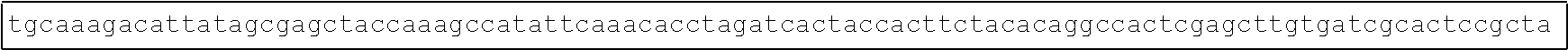

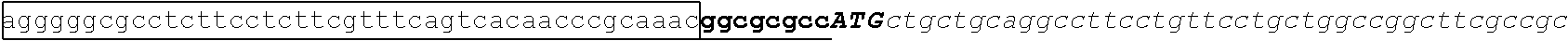

caagatcagcgcctccatgacgaacgagacgtccgaccgccccctggtgcacttcacccccaacaagggctggatgaacgaccccaacgg
cctgtggtacgacgagaaggacgccaagtggcacctgtacttccagtacaacccgaacgacaccgtctgggggacgccatgttctggggcc
acgccacgtccgacgacctgaccaactgggaggaccagcccatcgccatcgccccgaagcgcaacgactccggcgccuctccggctccat
ggtggtggactacaacaacacctccggcttcttcaacgacaccatcgacccgcgccagcgctgcgtggccatctggacctacaacaccccgg
agtccgaggagcagtacatctcctacagcctggacggcggctacaccttcaccgagtaccagaagaaccccgtgctggccgccaactccac
ccagttccgcgacccgaaggtcttctggtacgagccctcccagaagtggatcatgaccgcggccaagtcccaggactacaagatcgagatct
actcctccgacgacctgaagtcctggaagctggagtccgcgttcgccaacgagggcttcctcggctaccagtacgagtgccccggcctgatcg
aggtccccaccgagcaggaccccagcaagtcctactgggtgatgttcatctccatcaaccccggcgccccggccggcggctcatcaaccagt
acttcgtcggcagcttcaacggcacccacttcgaggccttcgacaaccagtcccgcgtggtggacttcggcaaggactactacgccctgcaga
ccttcttcaacaccgacccgacctacgggagcgccctgggcatcgcgtgggcctccaactgggagtactccgccttcgtgcccaccaacccct
ggcgctcctccatgtccctcgtgcgcaagttctccctcaacaccgagtaccaggccaacccggagacggagctgatcaacctgaaggccgag
ccgatcctgaacatcagcaacgccggcccctggagccggttcgccaccaacaccacgttgacgaaggccaacagctacaacgtcgacctgt
ccaacagcaccggcaccctggagttcgagctggtgtacgccgtcaacaccacccagacgatctccaagtccgtgttcgcggacctctccctct
ggttcaagggcctggaggaccccgaggagtacctccgcatgggcttcgaggtgtccgcgtcctccttcttcctggaccgcgggaacagcaag
gtgaagttcgtgaaggagaacccctacttcaccaaccgcatgagcgtgaacaaccagcccttcaagagcgagaacgacctgtcctactaca
aggtgtacggcttgctggaccagaacatcctggagctgtacttcaacgacggcgacgtcgtgtccaccaacacctacttcatgaccaccggga

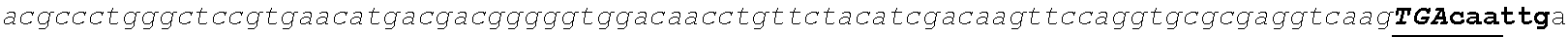

cgcccgcgcggcgcacctgacctgttctctcgagggcgcctgttctgccttgcgaaacaagcccctggagcatgcgtgcatgatcgtctctggcgc
cccgccgcgcggtttgtcgccctcgcgggcgccgcggccgcgggggcgcattgaaattgttgcaaaccccacctgacagattgagggcccagg
caggaaggcgttgagatggaggtacaggagtcaagtaactgaaagtttttatgataactaacaacaaagggtcgtttctggccagcgaatgacaag
aacaagattccacatttccgtgtagaggcttgccatcgaatgtgagcgggcgggccgcggacccgacaaaacccttacgacgtggtaagaaaaac
gtggcgggcactgtccctgtagcctgaagaccagcaggagacgatcggaagcatcacagcacaggatcccgcgtctcgaacagagcgcgcag
aggaacgctgaaggtctcgcctctgtcgcacctcagcgcggcatacaccacaataaccacctgacgaatgcgcttggttcttcgtccattagcgaag
cgtccggttcacacacgtgccacgttggcgaggtggcaggtgacaatgatcggtggagctgatggtcgaaacgttcacagcctagggcagcagc
agctcggatagtatcgacacactctggacgctggtcgtgtgatggactgttgccgccacacttgctgccttgacctgtgaatatccctgccgcttttatc
aaacagcctcagtgtgtttgatcttgtgtgtacgcgcttttgcgagttgctagctgcttgtgctatttgcgaataccacccccagcatccccttccctcgtt
tcatatcgcttgcatcccaaccgcaacttatctacgctgtcctgctatccctcagcgctgctcctgctcctgctcactgcccctcgcacagccttggtttg
ggctccgcctgtattctcctggtactgcaacctgtaaaccagcactgcaatgctgatgcacgggaagtagtgggatgggaacacaaatggaaagct

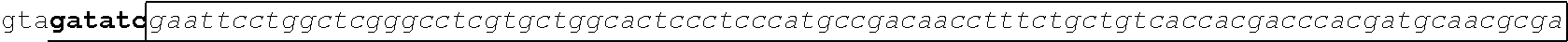

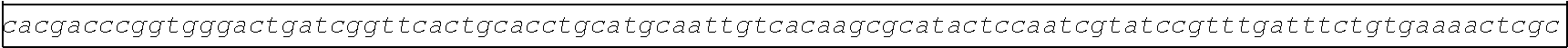

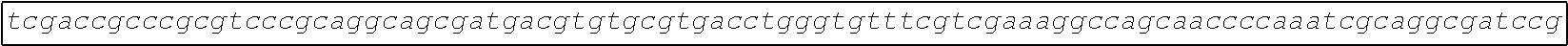

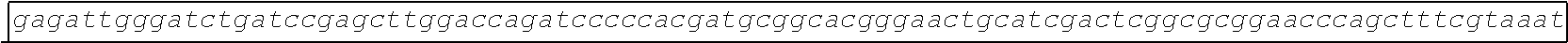

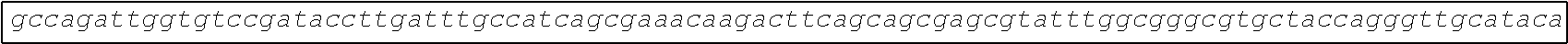

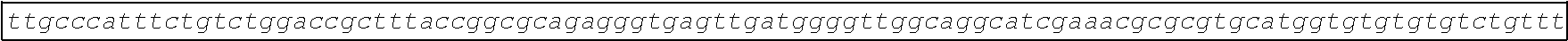

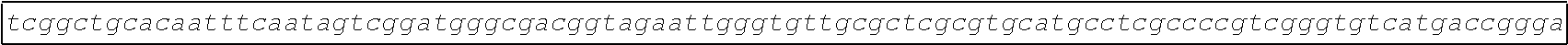

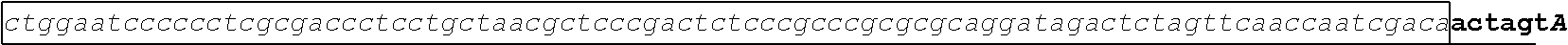

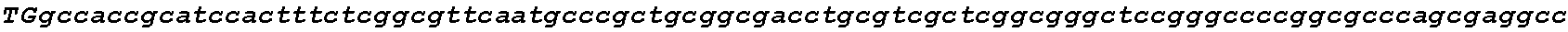

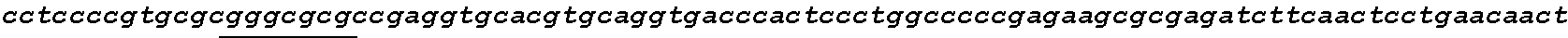

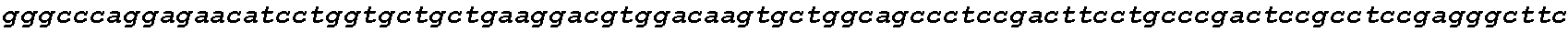

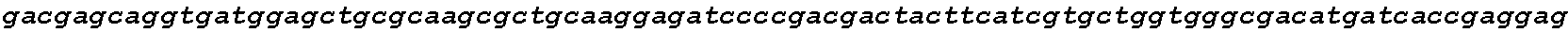

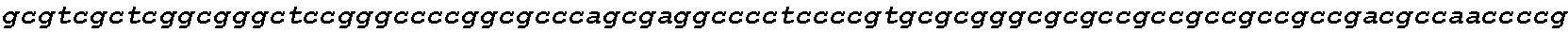

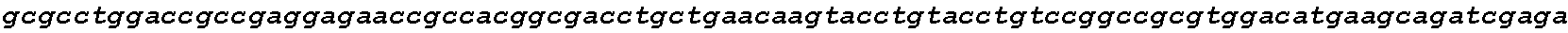

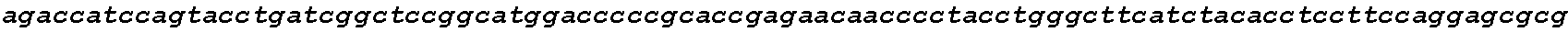

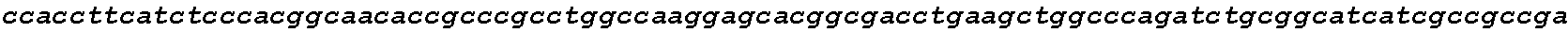

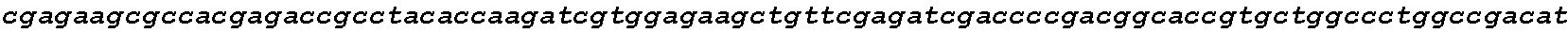

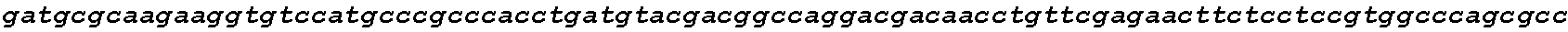

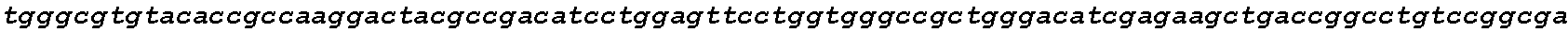

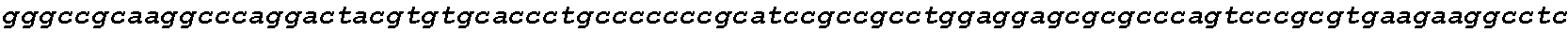

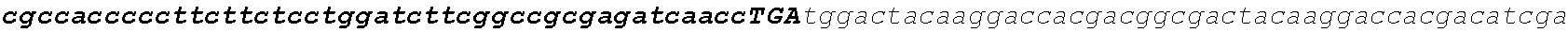

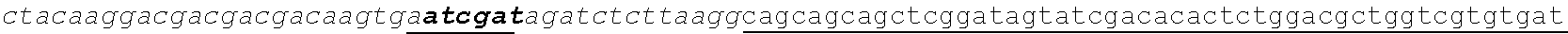

ggactgttgccgccacacttgctgccttgacctgtgaatatccctgccgcttttatcaaacagcctcagtgtgtttgatcttgtgtgtacgcgcttttgcga
gttgctagctgcttgtgctatttgcgaataccacccccagcatccccttccctcgtttcatatcgcttgcatcccaaccgcaacttatctacgctgtcctgc
tatccctcagcgctgctcctgctcctgctcactgcccctcgcacagccttggtttgggctccgcctgtattctcctggtactgcaacctgtaaaccagca
ctgcaatgctgatgcacgggaagtagtgggatgggaacacaaatggaaagcttaattaagagctcttgttttccagaaggagttgctccttgagc
ctttcattctcagcctcgataacctccaaagccgctctaattgtggagggggttcgaatttaaaagcttggaatgttggttcgtgcgtctggaa
caagcccagacttgttgctcactgggaaaaggaccatcagctccaaaaaacttgccgctcaaaccgcgtacctctgctttcgcgcaatctg
ccctgttgaaatcgccaccacattcatattgtgacgcttgagcagtctgtaattgcctcagaatgtggaatcatctgccccctgtgcgagccc
atgccaggcatgtcgcgggcgaggacacccgccactcgtacagcagaccattatgctacctcacaatagttcataacagtgaccatatttc
tcgaagctccccaacgagcacctccatgctctgagtggccaccccccggccctggtgcttgcggagggcaggtcaaccggcatggggcta
ccgaaatccccgaccggatcccaccacccccgcgatgggaagaatctctccccgggatgtgggcccaccaccagcacaacctgctggcc
caggcgagcgtcaaaccataccacacaaatatccttggcatcggccctgaattccttctgccgctctgctacccggtgcttctgtccgaagc
aggggttgctagggatcgctccgagtccgcaaacccttgtcgcgtggcggggcttgttcgagcttgaagagc.

### Example 18

### Expression of Ketoacyl-CoA Reductase (KCR), Hydroxyacyl-CoA Hydratase (HACD) and Enoyl-CoA Reductase (ECR)

In this example, the outcome of expression of Ketoacyl-CoA Reductase (KCR), Hydroxyacyl-CoA Dehydratase (HACD) and Enoyl-CoA Reductase (ECR), enzymes involved in very long chain fatty acid biosynthesis, in P. moriformis (UTEX 1435) is disclosed. Specifically, we demonstrate that expression of heterologous ECR, HACD or KCR genes from our internally assembled Crambe abyssinica transcriptome in Solazyme erucic strains S7211 and S7708 (discussed above) results in increases in both eicosenoic (C20:1) and erucic (C22:1) acids. The preparation of S7211 and S7708 are discussed in the Examples above.

Higher plants and most other eukaryotes have a highly specialized elongation system for extension of fatty acids beyond C18. Each elongation reaction condenses two carbons at a time from malonyl-CoA to an acyl group, followed by reduction, dehydration and a final reduction reaction. FAE (or KCS), a membrane bound protein localized in the cytosol, catalyzes the condensation of malonyl-CoA with an acyl group. Additional components of the elongation system have not been characterized in greater detail in higher plants. Having previously demonstrated the function of a heterologous FAE in P. moroformis (WO2013/158908, incorporated by reference), this example discloses the expression of heterologous KCR, HACD and ECR enzyme activities in strains already expressing a functional FAE gene. Arabidopsis KCR, HACD and ECR protein sequences were used as baits to mine the corresponding full-length genes from P. moriformis as well as our internally assembled Crambe abbysinica, Alliaria petiolata, Erysimum allioni, Crambe cordifolia and Erysimum golden gem transcriptomes. KCR, HACD and ECR genes identified from the P. moriformis transcriptome were found to be fairly divergent from their higher plant homologs. The sequence alignment of P. moriformis and higher plant KCR, HACD and ECR protein sequences are shown in FIGS. 3-5. Previously, we identified Crambe abyssinica FAE (KCS) as one of the best heterologous FAEs in our host, and thus we decided to codon optimize and synthesize the KCR, HACD and ECR genes from C. abyssinica and express them in S7211 (Crambe abyssinica FAE strain) and S7708 (Lunaria annua FAE strain). The sequence identities between P. moriformis KCR, HACD and ECR and the respective plant sequences are shown in Tables 100-102 below.

TABLE 100

| | | A thaliana | | | | | |
|---|---|---|---|---|---|---|---|
| | A petiolata E . . . | ECR | C abyssinica . . . | C cordofolia . . . | E allioni ECR | P moriformis . . . | P moriformis . . . |
| A petiolata ECR | | 96.1% | 97.4% | 97.7% | 97.4% | 47.6% | 47.6% |
| A thaliana ECR | 96.1% | | 96.8% | 97.1% | 97.4% | 47.3% | 47.3% |
| C abyssinica ECR | 97.4% | 96.8% | | 99.7% | 98.1% | 46.9% | 46.9% |
| C cordofolia ECR | 97.7% | 97.1% | 99.7% | | 98.4% | 47.3% | 47.3% |
| E allioni ECR | 97.4% | 97.4% | 98.1% | 98.4% | | 48.6% | 48.6% |
| P moriformis ECR1 | 47.6% | 47.3% | 46.9% | 47.3% | 48.6% | | 97.0% |
| P moriformis ECR2 | 47.6% | 47.3% | 46.9% | 47.3% | 48.6% | 97.0% | |

TABLE 101

| | A | A | C | C | E allioni | E | | |
|---|---|---|---|---|---|---|---|---|
| | petiolata H . . . | thaliana H . . . | abyssinica . . . | cordofolia . . . | HACD | golden ge . . . | E helvetium . . . | P moriformis . . . |
| A petiolata | | 97.3% | 94.6% | 94.1% | 99.1% | 99.1% | 100% | 40.3% |
| HACD | | | | | | | | |
| A thaliana | 97.3% | | 94.6% | 94.1% | 96.4% | 96.4% | 97.3% | 40.1% |
| HACD | | | | | | | | |
| C abyssinica | 94.6% | 94.6% | | 98.6% | 93.7% | 93.7% | 94.6% | 40.8% |
| HACD | | | | | | | | |
| C cordofolia | 94.1% | 94.1% | 98.6% | | 93.2% | 93.2% | 94.1% | 40.8% |
| HACD | | | | | | | | |
| E allioni | 99.1% | 96.4% | 93.7% | 93.2% | | 99.1% | 99.1% | 40.3% |
| HACD | | | | | | | | |
| E golden gem | 99.1% | 96.4% | 93.7% | 93.2% | 99.1% | | 99.1% | 39.9% |
| HACD | | | | | | | | |
| E helvetium | 100% | 97.3% | 94.6% | 94.1% | 99.1% | 99.1% | | 40.3% |
| HACD | | | | | | | | |
| P moriformis | 40.3% | 40.1% | 40.8% | 40.8% | 40.3% | 39.9% | 40.3% | |
| HACD1 | | | | | | | | |

TABLE 102

| | A petiolata | A thaliana | B napus | B napus | C | C | E allioni | P | Z mays |
|---|---|---|---|---|---|---|---|---|---|
| | K . . . | KCR | KCR1 | KCR2 | abyssinica . . . | cordofolia . . . | KCR | moriformis . . . | KCR |
| A petiolata | | 92.1% | 86.2% | 85.0% | 85.6% | 85.6% | 88.4% | 39.9% | 54.3% |
| KCR | | | | | | | | | |
| A thaliana | 92.1% | | 89.3% | 86.1% | 89.4% | 86.7% | 91.9% | 41.0% | 53.9% |
| KCR | | | | | | | | | |
| B napus | 86.2% | 89.3% | | 97.2% | 89.7% | 90.6% | 89.7% | 42.4% | 55.3% |
| KCR1 | | | | | | | | | |
| B napus | 85.0% | 88.1% | 97.2% | | 89.0% | 89.7% | 87.0% | 42.2% | 56.2% |
| KCR2 | | | | | | | | | |
| C abyssinica | 85.6% | 88.4% | 89.7% | 89.0% | | 96.6% | 90.6% | 41.5% | 55.3% |
| KCR | | | | | | | | | |
| C cordofolia | 85.6% | 68.7% | 90.6% | 89.7% | 96.6% | | 91.5% | 41.8% | 55.9% |
| KCR1 | | | | | | | | | |
| E allioni | 88.4% | 91.5% | 89.7% | 87.0% | 90.6% | 91.5% | | 42.7% | 55.0% |
| KCR | | | | | | | | | |
| P moriformis | 39.9% | 41.0% | 42.4% | 42.7% | 41.5% | 41.8% | 42.7% | | 41.2% |
| KCR1-1 | | | | | | | | | |
| Z mays | 54.3% | 53.9% | 55.3% | 56.2% | 55.3% | 55.9% | 55.0% | 41.2% | |
| KCR | | | | | | | | | |

Construct Used for the Expression of the Crambe abyssinica Enoyl-CoA Reductase (CrhECR) in Erucic Strains S7211 and S7708—[pSZ5907]

Strains S7211 and S7708, transformed with the construct pSZ5907, were generated, which express Sacharomyces carlbergenesis MEL1 gene (allowing for their selection and growth on medium containing melibiose) and C. abyssinica ECR gene targeted at endogenous PmFAD2-1 genomic region. Construct pSZ5907 introduced for expression in S7211 and S7708 can be written as:
pSZ5907: FAD2-1-1 5′ flank::PmHXT1-ScarMEL1-CvNR:Buffer DNA:PmSAD2-2v2-CrhECR-CvNR::FAD2-1 3′ flank.

The sequence of the transforming DNA is provided below. Relevant restriction sites in the construct are indicated in lowercase, underlined bold, and are from 5′-3′ NdeI, KpnI, SpeI, SnaBI, EcoRI, SpeI, XhoI, SacI and XbaI, respectively. NdeI and XbaI sites delimit the 5′ and 3′ ends of the transforming DNA. Bold, lowercase sequences represent genomic DNA from S3150 that permit targeted integration at the FAD2-1 locus via homologous recombination. Proceeding in the 5′ to 3′ direction, the endogenous P. moriformis Hexose Transporter 1 v2 promoter driving the expression of the S. carlbergenesis MEL1 gene (encoding an alpha galactosidase enzyme activity required for catabolic conversion of Melibise to glucose and galactose, thereby permitting the transformed strain to grow on melibiose) is indicated by lowercase, boxed text. Uppercase italics indicate the initiator ATG and terminator TGA for MEL1, while the coding region is indicated with lowercase italics. The P. moriformis Phosphoglucokinase (PGK) gene 3′ UTR is indicated by lowercase underlined text followed by buffer/spacer DNA sequence indicated by lowercase bold italic text Immediately following the buffer DNA is an endogenous SAD2-2 promoter of P. moriformis, indicated by boxed italicized text. Uppercase, bold italics indicate the Initiator ATG and terminator TGA codons of the CrhECR, while the lowercase italics indicate the remainder of the gene. The C. vulgaris nitrate reductase 3′ UTR is indicated by lowercase underlined text followed by the S3150 FAD2-1 genomic region indicated by bold, lowercase text. The final construct was sequenced to ensure correct reading frames and targeting sequences.

Nucleotide sequence of transforming DNA contained in plasmid pSZ5907:
(SEQ ID NO: 142).
catatgcggtgtgaaataccgcacagatgcgtaaggagaaaataccgcatcaggcgccattcgccattcaggctgcgcaactgttgg
gagggcgatcggtgcgggcctcttcgctattacgccagctggcgaaaggggatgtgctgcaaggcgattaagttgggtaacgcc
agggttttcccagtcacgacgttgtaaaacgacggccagtgaattgatgatgctcttcgcgaaggtcattttccagaacaacgacca
tggcttgtcttagcgatcgctcgaatgactgctagtgagtcgtacgctcgacccagtcgctcgcaggagaacgcggcaactgcc
gagcttcggcttgccagtcgtgactcgtatgtgatcaggaatcattggcattggtagcattataattcggcttccgcgctgtttat
gggcatggcaatgtctcatgcagtcgaccttagtcaaccaattctgggtggccagctccgggcgaccgggctccgtgtcgccg
ggcaccacctcctgccatgagtaacagggccgccctctcctcccgacgttggcccactgaataccgtgtcttggggccctacat
gatgggctgcctagtcgggcgggacgcgcaactgcccgcgcaatctgggacgtggtctgaatcctccaggcgggtttccccga
gaaagaaagggtgccgatttcaaagcagagccatgtgccgggccctgtggcctgtgttggcgcctatgtagtcaccccccctc
acccaattgtcgccagtttgcgcaatccataaactcaaaactgcagcttctgagctgcgctgttcaagaacacctctggggtttg

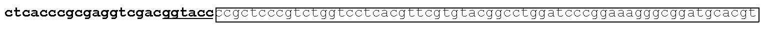

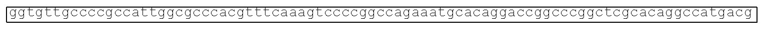

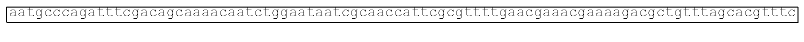

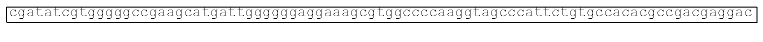

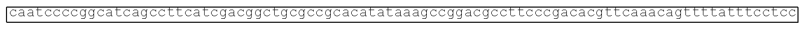

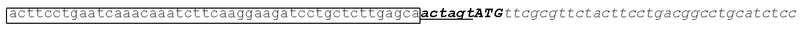

ctgagggcgtgttcggcgtctccccctcctacaacggcctgggcctgacgccccagatgggctgggacaactggaacacgttcg
cctgcgacgtctccgagcagctgctgctggacacggccgaccgcatctccgacctgggcctgaaggacatgggctacaagtaca
tcatcctggacgactgctggtcctccggccgcgactccgacggcttcctggtcgccgacgagcagaagttccccaacggcatggg
ccacgtcgccgaccacctgcacaacaactcttcctgttcggcatgtactcctccgcgggcgagtacacgtgcgccggctaccccg
gctccctgggccgcgaggaggaggacgcccagttcttcgcgaacaaccgcgtggactacctgaagtacgacaactgctacaac
aagggccagttcggcacgcccgagatctcctaccaccgctacaaggccatgtccgacgccctgaacaagacgggccgccccat
cttctactccctgtgcaactggggccaggacctgaccttctactggggctccggcatcgcgaactcctggcgcatgtccggcgacgt
cacggcggagttcacgcgccccgactcccgctgcccctgcgacggcgacgagtacgactgcaagtacgccggcttccactgctc
catcatgaacatcctgaacaaggccgcccccatgggccagaacgcgggcgtcggcggctggaacgacctggacaacctggag
gtcggcgtcggcaacctgacggacgacgaggagaaggcgcacttctccatgtgggccatggtgaagtcccccctgatcatcggc
gcgaacgtgaacaacctgaaggcctcctcctactccatctactcccaggcgtccgtcatcgccatcaaccaggactccaacggca
tccccgccacgcgcgtctggcgctactacgtgtccgacacggacgagtacggccagggcgagatccagatgtggtccggccccc
tggacaacggcgaccaggtcgtggcgctgctgaacggcggctccgtgtcccgccccatgaacacgaccctggaggagatcttctt
cgactccaacctgggctccaagaagctgacctccacctgggacatctacgacctgtgggcgaaccgcgtcgacaactccacggc
gtccgccatcctgggccgcaacaagaccgccaccggcatcctgtacaacgccaccgagagtcctacaaggacggcctgtcca
agaacgacacccgcctgttcggccagaagatcggctccctgtcccccaacgcgatcctgaacacgaccgtccccgcccacggc

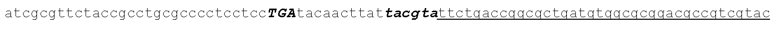

tctttcagactttactcttgaggaattgaacctttctcgcttgctggcatgtaaacattggcgcaattaattgtgtgatgaagaaagggtggc
acaagatggatcgcgaatgtacgagatcgacaacgatggtgattgttatgaggggccaaacctggctcaatcttgtcgcatgtccggc
gcaatgtgatccagcggcgtgactctcgcaacctggtagtgtgtgcgcaccgggtcgctttgattaaaactgatcgcattgccatcccgt
caactcacaagcctactctagctcccattgcgcactcgggcgcccggctcgatcaatgttctgagcggagggcgaagcgtcaggaa

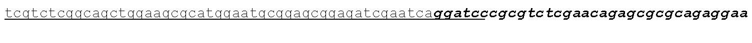

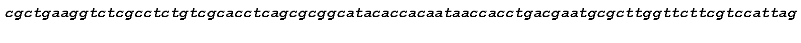

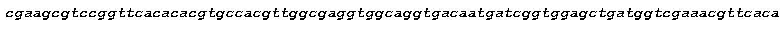

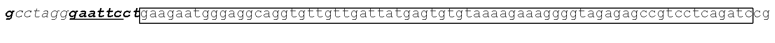

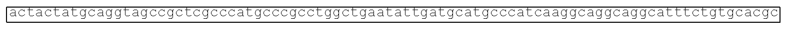

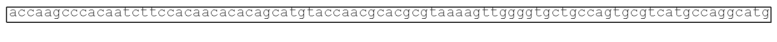

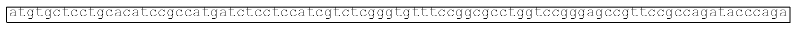

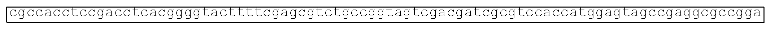

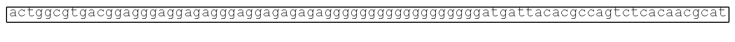

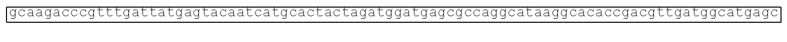

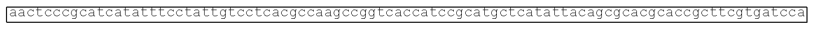

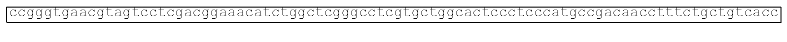

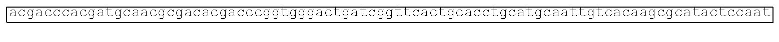

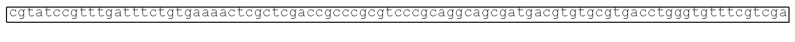

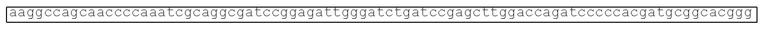

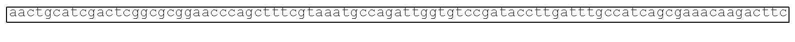

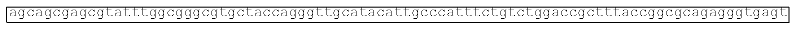

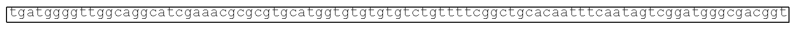

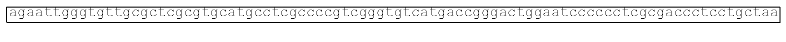

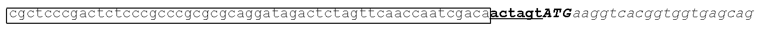

gtccggcagggaggtgctcaaggcccccctggacctgccggactccgccacggtgcgctgacctccaggaggccttccacaagc
gcgcgaagaagttttatcccagccgccagcggctgaccctgccggtggcccccggctccaaggacaagccggtggtgctgaact
cgaagaagagcctcaaggagtactgcgacggtaacaccgactcgctcacggtggtgtttaaggacttgggcgcgcaggtctcct
accgcaccctgttcttcttcgagtaactgggccccctgctgatctaccccgtcttctactacttccctgtctataagtacctgggctacgg
cgaggaccgcgtcatccacccggtgcagacgtatgccatgtactactggtgcttccactacttttaagcgattatggagacgttcttc
gtgcaccgcttcagccacgccacctcgcccatcggtaacgtcttccgcaactgcgcctactactggacgttcggcgcctacatcgct
tactacgtgaaccaccccctgtacaccccctgtgagcgacttgcagatgaagatcggcttcgggttcggcctcgtgtttcaggtggcg
aacttctactgccacatcctgctgaagaatctgcgcgacccgaacggcagcggcggttaccagatcccgcgcggcttcctgttcaa
catcgtcacgtgcgcgaactacaccacggagatctaccagtggctcggctttaacatcgccacgcagaccatcgccggctacgtg
ttcctcgcggtggccgccctgattatgaccaactgggccctcggcaagcactcgcggctccggaagatcttcgacggcaaggacg

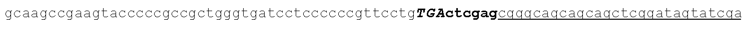

cacactctggacgctggtcgtgtgatggactgttgccgccacacttgctgccttgacctgtgaatatccctgccgcttttatcaaacagcc
tcagtgtgtttgatcttgtgtgtacgcgcttttgcgagttgctagctgcttgtgctatttgcgaataccacccccagcatccccttccctcgttt
catatcgcttgcatcccaaccgcaacttatctacgctgtcctgctatccctcagcgctgctcctgctcctgctcactgcccctcgcacagc
cttggtttgggctccgcctgtattctcctggtactgcaacctgtaaaccagcactgcaatgctgatgcacgggaagtagtgggatggga
acacaaatggaagctgtagagctcctcactcagcgcgcctgcgcggggatgcggaacgccgccgccgccttgtcttttgcacgc
gcgactccgtcgcttcgcgggtggcacccccattgaaaaaaacctcaattctgtttgtggaagacacggtgtacccccaaccac
ccacctgcacctctattattggtattattgacgcgggagcgggcgttgtactctacaacgtagcgtctctggttttcagctggctc
ccaccattgtaaattcttgctaaaatagtgcgtggttatgtgagaggtatggtgtaacagggcgtcagtcatgttggttttgtgc
tgatctcgggcacaaggcgtcgtcgacgtgacgtgcccgtgatgagagcaataccgcgctcaaagccgacgcatggcctttac
tccgcactccaaacgactgtcgctcgtatttttcggatatctattttttaagagcgagcacagcgccgggcatgggcctgaaagg
cctcgcggccgtgctcgtggtgggggccgcgagcgcgtggggcatcgcggcagtgcaccaggcgagacggaggaacgcat
ggtgagtgcgcatcacaagatgcatgtcttgttgtctgtactataatgctagagcatcaccaggggcttagtcatcgcacctgct
ttggtcattacagaaattgcacaagggcgtcctccgggatgaggagatgtaccagctcaagctggagcggcttcgagccaag
caggagcgcggcgcatgacgacctacccacatgcgaagagcctctaga

Constructs Used for the Expression of the Crambe abyssinica Hydroxyacyl-CoA Hydratase (HACD) and Ketoacyl-CoA Reductase (KCR) Genes in S7211 and S7708

In addition to the C. abyssinica KCR targeted at FAD2-1 locus (pSZ5909), C. abyssinica ECR targeted at FAD2-1 locus (pSZ5907) and C. abyssinica HACD targeted at FAD2-1 locus (pSZ5908) have been constructed for expression in S7211 and S7708. These constructs can be described as:
pSZ5908—FAD2-1-1 5′::PmHXT1-ScarMEL1-CvNR:Buffer DNA:PmSAD2-2v2-CrhHACD-CvNR::FAD2-1 3′
pSZ5909—FAD2-1-1 5 ‘::PmHXT1-ScarMEL1-CvNR:Buffer DNA:PmSAD2-2v2-CrhKCR-CvNR::FAD2-1 3’

Both of these constructs have the same vector backbone; selectable marker, promoters, and 3′ utr as pSZ5907, except that CrhECR was replaced with CrHACD or CrKCR, respectively. Relevant restriction sites in these constructs are also the same as in pSZ5907. The nucleotide sequences of CrhHACD and CrhKCR are shown below. Relevant restriction sites, as bold text, are shown 5′-3′ respectively.

CrhHACD gene in pSZ5908:
(SEQ ID NO: 143)

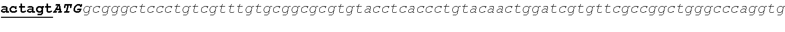

ctgtactttgccgtcaagacgctcaaggagtccggccacgagaacgtgtacgacgccgtggagaagcccctccagctggcgcaaac
cgccgcggtcctggagatcctccacggcctggtcggcctcgtcaggagcccggtctcggccaccctgccgcagatcgggagccgc
ctctttctgacctggggcattctgtattccttcccggaggtccagagccactttctggtgacctccctcgtgatcagctggtcgatcacgg
aaatcatccgctacagcttcttcggcctgaaggaggcgctgggcttcgcgcccagctggcacctgtggctccgctattcgagctttctg
gtgctctaccccaccggcatcacctccgaggtcggcctcatctacctggccctgccgcacatcaagacgtcggagatgtactccgtcc
gcatgcccaacaccttgaacttttccttcgactttttctacgccacgattctcgtcctcgcgatctacgtccccggttcgccccacatgtacc

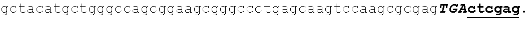

CrhKCR gene in pSZ5909:
(SEQ ID NO: 144)

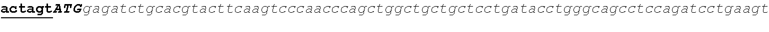

cgacgttctccctcctgaagagcctgtacatctacttcctgcgccccggcaagaacctccgccgctacgggtcctgggccattatcacc
ggcccgaccgacggcatcggcaaggcctttgcgttccagctggcccacaagggcctgaacctggtgctggtggcgcgcaacccgg
acaagctgaaggacgtctccgacagcatcaggtccaagcatagcaacgtgcagatcaagacggtgatcatggactttagcggcgac
gttgacgacggcgtccgccgcatcaaggagaccatcgaggggctggaggtgggcatcctgatcaacaatgccggcatgtcctaccc
gtacgcgaagtactttcacgaggtcgacgaggagctcgtcaacggcctcatcaaaatcaacgtcgagggcacgaccaaggtgaccc
aggccgtgctgccgggcatgctggagcgcaagcgcggcgccatcgtcaacatgggcagcggcgcggccgccctgatcccgtcgt
accccactacagcgtgtatgccggcgcgaagacgtacgtggaccagttcacccggtgcctgcacgtcgagtacaagaagagcggc
attgacgtccagtgccaggtcccgctctacgtggccacgaagatgacgaagatccgccgcgcctccacctggtcgcctcccccgag
ggctacgccaaggccgccctgcggttcgtggggtacgaggcccggtgcaccccctactggccgcacgccctgatgggctacgtcgt
ctccgccctgccccagtccgtgacgagtcatcaacatcaagcgctgcctgcagatccgcaagaagggcatgctgaaggattcgcgg

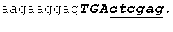

Expression of CrhKCR Gene in pSZ5909

To determine their impact on fatty acid profiles, all the three constructs described above were transformed independently into either S7211 or S7708. Primary transformants were clonally purified and grown under standard lipid production conditions at pH7.0. Strains S7211 and S7708 express a FAE, from C. abyssinica or L. annua respectively, under the control of pH regulated, AMT03 (Ammonium transporter 03) promoter. Thus, both parental (S7211 and S7708) and the resulting KCR, ECR and HACD transformed strains require growth at pH 7.0 to allow for maximal fatty acid elongase (FAE) gene expression. The resulting profiles from a set of representative clones arising from transformations with pSZ5907 (D4905), pSZ5908 (D4906) and pSZ5909 (D4907) into S7708 and S7211 are shown in Tables 103-105, respectively. In both S7708 and S7211, expression of CrhECR, CrhHACD or CrhKCR leads to an increase in both C20:1 and C22:1 content.

TABLE 103
Fatty acid profiles of S7708 and S7211 strains transformed
with D4905 (CrhECR).

| Sample ID | C18:1 | C18:2 | C18:3α | C20:1 | C22:1 |
|---|---|---|---|---|---|
| S7708; pH 7 | 49.41 | 8.89 | 0.64 | 2.90 | 1.53 |
| S7211; pH 7 | 46.64 | 11.16 | 0.79 | 4.76 | 1.84 |
| S7708; T1379; | 43.04 | 11.15 | 1.00 | 3.50 | 2.71 |
| D4905-9; pH 7 | | | | | |
| S7708; T1379; | 52.86 | 8.21 | 0.73 | 3.34 | 1.95 |
| D4905-35; pH 7 | | | | | |
| S7708; T1379; | 52.75 | 8.19 | 0.74 | 3.31 | 1.93 |
| D4905-31; pH 7 | | | | | |
| S7708; T1379; | 52.72 | 8.18 | 0.73 | 3.31 | 1.89 |
| D4905-25; pH 7 | | | | | |
| S7708; T1379; | 47.35 | 9.45 | 0.74 | 3.06 | 1.83 |
| D4905-10; pH 7 | | | | | |
| S7211; T1380; | 47.28 | 9.20 | 0.78 | 5.26 | 2.06 |
| D4905-4; pH 7 | | | | | |
| S7211; T1380; | 47.53 | 10.42 | 0.76 | 4.97 | 1.91 |
| D4905-3; pH 7 | | | | | |
| S7211; T1380; | 48.36 | 8.75 | 0.74 | 5.01 | 1.83 |
| D4905-5; pH 7 | | | | | |
| S7211; T1380; | 47.43 | 8.52 | 0.77 | 4.88 | 1.75 |
| D4905-1; pH 7 | | | | | |

TABLE 104
Fatty acid profiles of S7708 and S7211 strains transformed
with D4906 (CrhHACD)

| Sample ID | C18:1 | C18:2 | C18:3α | C20:1 | C22:1 |
|---|---|---|---|---|---|
| S7708; pH 7 | 49.41 | 8.89 | 0.64 | 2.90 | 1.53 |
| S7211; pH 7 | 46.64 | 11.16 | 0.79 | 4.76 | 1.84 |
| S7708; T1379; | 46.83 | 8.68 | 0.65 | 3.87 | 2.20 |
| D4906-2; pH 7 | | | | | |
| S7708; T1379; | 50.82 | 6.78 | 0.60 | 3.82 | 2.00 |
| D4906-7; pH 7 | | | | | |
| S7708; T1379; | 47.88 | 8.64 | 0.61 | 3.56 | 1.99 |
| D4906-4; pH 7 | | | | | |
| S7708; T1379; | 49.99 | 6.97 | 0.64 | 3.70 | 1.97 |
| D4906-8; pH 7 | | | | | |
| S7708; T1379; | 49.83 | 6.96 | 0.62 | 3.62 | 1.91 |
| D4906-11; pH 7 | | | | | |
| S7211; T1380; | 45.58 | 8.95 | 0.81 | 5.87 | 2.40 |
| D4906-2; pH 7 | | | | | |
| S7211; T1380; | 45.73 | 8.90 | 0.80 | 5.72 | 2.28 |
| D4906-1; pH 7 | | | | | |
| S7211; T1380; | 46.91 | 10.22 | 0.80 | 5.02 | 1.90 |
| D4906-3; pH 7 | | | | | |
| S7211; T1380; | 46.68 | 10.61 | 0.77 | 4.77 | 1.77 |
| D4906-4; pH 7 | | | | | |

TABLE 105
Fatty acid profiles of S7708 and S7211 strains transformed
with D4907 (CrhKCR).

| Sample ID | C18:1 | C18:2 | C18:3α | C20:1 | C22:1 |
|---|---|---|---|---|---|
| S7708; pH 7 | 49.41 | 8.89 | 0.64 | 2.90 | 1.53 |
| S7211; pH 7 | 46.64 | 11.16 | 0.79 | 4.76 | 1.84 |
| S7708; T1379; | 46.11 | 9.62 | 0.62 | 3.93 | 2.86 |
| D4907-7; pH 7 | | | | | |
| S7708; T1379; | 47.52 | 9.09 | 0.62 | 4.07 | 2.60 |
| D4907-6; pH 7 | | | | | |
| S7708; T1379; | 49.27 | 6.82 | 0.62 | 4.15 | 2.57 |
| D4907-2; pH 7 | | | | | |
| S7708; T1379; | 49.45 | 6.75 | 0.59 | 4.08 | 2.47 |
| D4907-4; pH 7 | | | | | |
| S7708; T1379; | 48.05 | 8.99 | 0.62 | 3.81 | 2.32 |
| D4907-9; pH 7 | | | | | |
| S7211; T1380; | 45.61 | 8.94 | 0.85 | 5.91 | 2.66 |
| D4907-7; pH 7 | | | | | |
| S7211; T1380; | 46.73 | 8.71 | 0.79 | 5.90 | 2.46 |
| D4907-6; pH 7 | | | | | |
| S7211; T1380; | 44.94 | 10.98 | 0.81 | 5.49 | 2.44 |
| D4907-3; pH 7 | | | | | |
| S7211; T1380; | 47.54 | 8.73 | 0.75 | 5.85 | 2.42 |
| D4907-2; pH 7 | | | | | |
| S7211; T1380; | 46.58 | 9.11 | 0.76 | 5.76 | 2.41 |
| D4907-4; pH 7 | | | | | |

### Example 19

### Expression of Acetyl-CoA Carboxylase (ACCase)

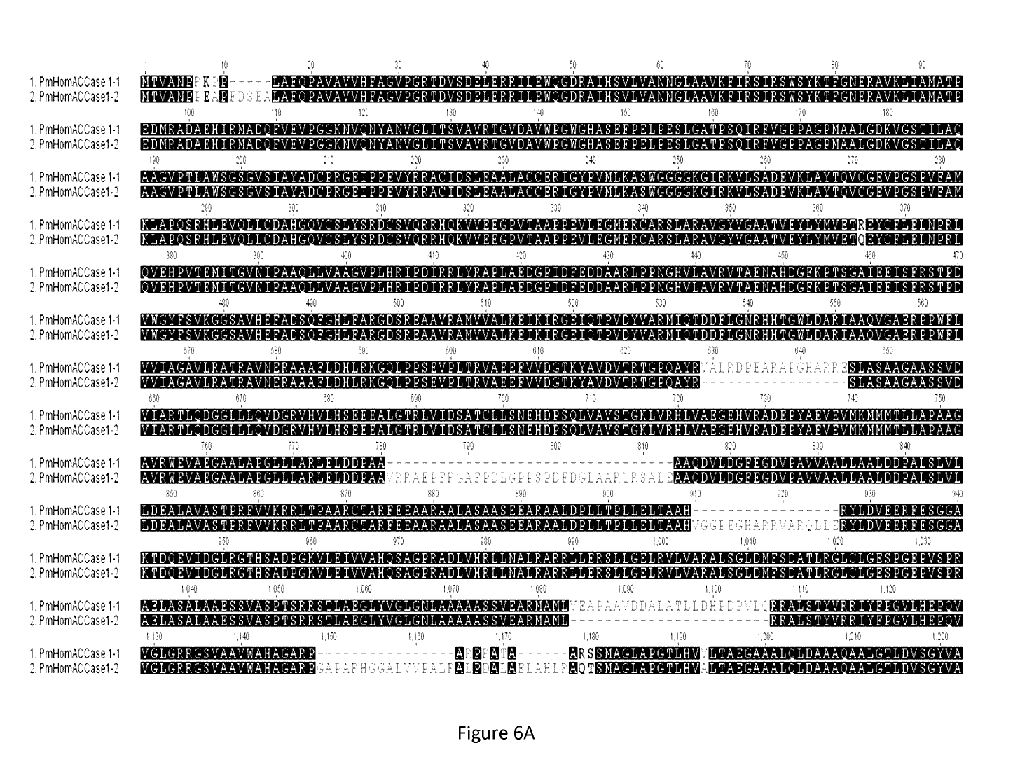

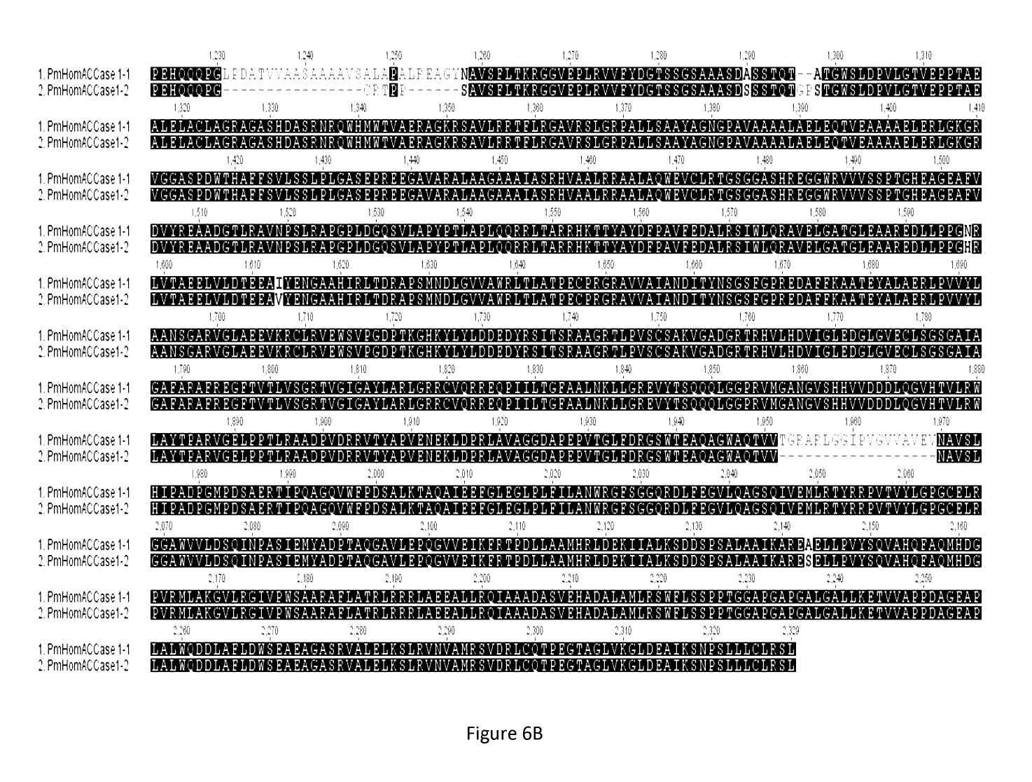

In this example, we demonstrate that upregulating cytosolic homomeric Acetyl-CoA carboxylase (ACCase) in erucic strains S7708 and S8414 results in a three or more fold increase in C22:1 content in the resulting transgenic strains. S7708 is a strain that expresses a Lunaria annua fatty acid elongase as discussed above and prepared according to co-owned WO2013/158938. Strain S8414 is an isolate that expresses a Crambe hispanica fatty acid elongase/3-ketoacyl-CoA synthase (FAE/KCS) and is recombinantly identical to S7211 (Example 10). Extension of fatty acids beyond C18, in microalgae, requires the coordinated action of four key cytosolic/ER enzymes—a Ketoacyl Co-A synthase (KCS aka fatty acid elongase, FAE), a Ketoacyl-CoA Reductase (KCR), a Hydroxyacyl-CoA Hydratase (HACD) and an Enoyl-CoA Reductase (ECR). Each elongation reaction condenses two carbons at a time from malonyl-CoA to an acyl group, followed by reduction, dehydration and a final reduction reaction. KCS (or FAE) catalyzes the condensation of malonyl-CoA with an acyl primer. Malonyl-CoA is generated through irreversible carboxylation of cytosolic acetyl-CoA by the action of multidomain cytosolic homomeric ACCase. For efficient and sustained fatty acid elongation, unavailability of ample malonyl-CoA can become a bottleneck. In the microalgal cell, malonyl-CoA is also used for the production of falvonoids, anthocyanins, malonated D-aminoacids and malonyl-amino cyclopropane-carboxylic acid, which further decreases its availability for fatty acid elongation. Using a bioinformatics approach we identified both alleles for ACCase in P. moriformis. PmACCase1-1 encodes a 2250 amino acid protein while PmACCase1-2 encodes a 2540 amino acid protein. The pairwise protein alignment of PmACCase1-1 and PmACCase1-2 is shown in FIGS. 6A and 6B. Given the large size of the protein we decided to hijack the endogenous ACCAse promoter with our strong pH regulatable Ammonia transport 3 (PmAMT03) promoter in S7708 and S8414. The “promoter hijack” was accomplished by inserting the AMT03 promoter between the endogenous PmACCCase1-1 or PmACCase 1-2 promoter and the initiation codon of the PmACCase1-1 or PmACCase1-2 protein in both S7708 and S8414, thus disrupting the endogenous promoter and replacing it with the Prototheca moriformis AMT03 promoter. This results in the expression the P. moriformis ACCase driven by the AMT03 promoter rather than the endogenous promoter. In S7708 transgenics both the LaFAE and the hijacked ACCase are driven by AMT03 promoter. The AMT03 promoter is a promoter that drives expression at pH 7 and at pH 5 expression is minimal. In S8414 the CrhFAE is driven by the PmSAD2-2v2 promoter, which is not a pH regulated promoter, and thus the effect of PmACCase can be easily monitored by running the lipid assays at either pH7. The amino acid alignment of P. moriformis ACCase1-1 and P. moriformis ACCase 1-2 is shown in FIGS. 6A and 6B. The sequence identity between P. moriformis ACCase 1-1 and a-2 is 92.3%.

Construct Used for the Upregulation of P. Moriformis Acetyl-CoA Carboxylase (PmACCase) in Erucic Strain and S7708 is pSZ5391.

Strain S7708, transformed with the construct pSZ5391, was generated, which expresses Sacharomyces carlbergenesis MEL1 gene (allowing for their selection and growth on medium containing melibiose) and upregulated P. morformis ACCase driven by a PmAMT03 promoter. Construct pSZ5391 introduced for expression in S7708 can be written as:

PmACCase1-1::PmHXT1v2-ScarMEL1-PmPGK:BDNA:PmAMT03::PmACCase1-1.

The sequence of the transforming DNA is provided below. Relevant restriction sites in the construct are indicated in lowercase, underlined bold, and are from 5′-3′ BsaBI, KpnI, SpeI, SnaBI, BamHI, EcoRI, SpeI and SbfI respectively. BasBI and SbfI sites delimit the 5′ and 3′ ends of the transforming DNA. Bold, lowercase sequences represent genomic DNA from S3150 that permit targeted integration at the ACCase locus via homologous recombination. Proceeding in the 5′ to 3′ direction, the endogenous P. moriformis Hexose Transporter 1 v2 promoter driving the expression of the S. carlbergenesis MEL1 gene (encoding an alpha galactosidase enzyme activity required for catabolic conversion of Meliobise to glucose and galactose, thereby permitting the transformed strain to grow on melibiose) is indicated by lowercase, boxed text. Uppercase italics indicate the initiator ATG and terminator TGA for MEL1, while the coding region is indicated with lowercase italics. The P. moriformis Phosphoglucokinase (PGK) gene 3′ UTR is indicated by lowercase underlined text followed by buffer/spacer DNA sequence indicated by lowercase bold italic text. Immediately following the buffer DNA is an endogenous AMT03 promoter of P. moriformis, indicated by boxed lowercase text followed by the PmACCCase1-1 genomic region indicated by bold, lowercase text. Uppercase, bold italics indicate the Initiator ATG of the endogenous PmACCase1-1 gene targeted for upregulation by preceding PmAMT03 promoter. The final construct was sequenced to ensure correct reading frames and targeting sequences.

Nucleotide sequence of transforming DNA contained in plasmid pSZ5391
transformed into S7708:
(SEQ ID NO: 145)
gatttctatcatcaagtttctcatatgtttcacgcgttgctcacaacaccggcaaatgcgttgttgttccctgtttttacaccttgcc
agagcctggtcaaagcttgacagtttgaccaaattcaggtggcctcatctctctcgcactgatagacattgcagatttggaaga
cccagtcagtacactacatgcacagccgtttgctcctgcgccatgaacttgccacttttgtgcgccggtcgggggtgatagctcg
gcagccgccgatcccaaaggtcccgcggcccaggggcacgagaacccccgacacgattaaatagccaaaatcagttagaac
ggcacctccaccctacccgaatctgacagggtcatcaagcgcgcgaaacaacggcgagggtgcgttcgggaagcgcgcgta
gttgacgcaagaagcctgggtcaggctgggagggccgcgagaagatcgcttcctgccgagtctgcacccacgcctcgagcgc
accgtccgcgaacaaccaacccctttgcgcgagccctgacattctttcaattgccaaggatgcacatgtgacacgtatagccat
tcggctttgtttgtgcctgcttgactcgcgtcatttaattgatttgtgccggtgagccgggagtcggccactcgtctccgagccgc
agtcccggcgccagtcccccggcctctgatctgggtccggaagggttggtataggagcggtctcggctatctgaagcccattac

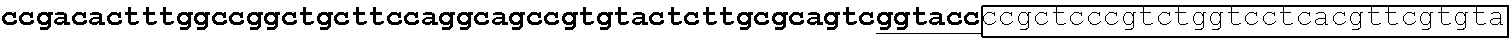

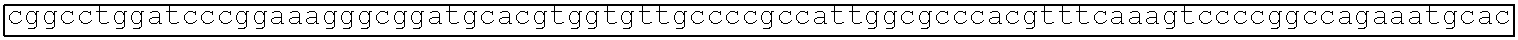

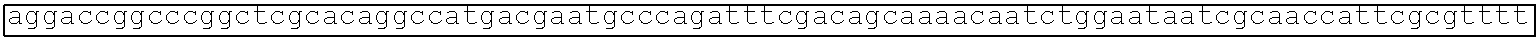

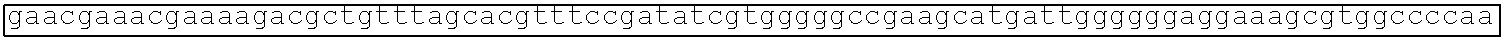

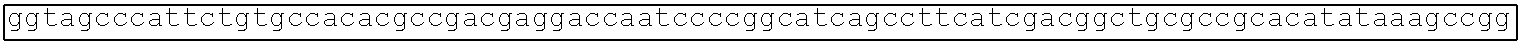

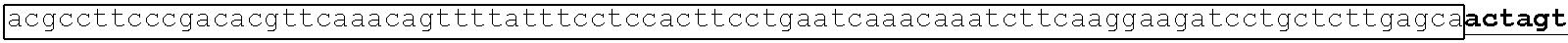

ATGttcgcgttctacttcctgacggcctgcatctccctgaagggcgtgtttggcgtctccccctcctacaacggcctgggcctgacg
ccccagatgggctgggacaactggaacacgttcgcctgcgacgtctccgagcagctgctgctggacacggccgaccgcatctcc
gacctgggcctgaaggacatgggctacaagtacatcatcctggacgactgctggtcctccggccgcgactccgacggcttcctgg
tcgccgacgagcagaagttccccaacggcatgggccacgtcgccgaccacctgcacaacaactccttcctgttcggcatgtactc
ctccgcgggcgagtacacgtgcgccggctaccccggctccctgggccgcgaggaggaggacgcccagttcttcgcgaacaacc
gcgtggactacctgaagtacgacaactgctacaacaagggccagttcggcacgcccgagatctcctaccaccgctacaaggcc
atgtccgacgccagaacaagacgggccgccccatatctactccctgtgcaactggggccaggacctgaccttctactggggctc
cggcatcgcgaactcctggcgcatgtccggcgacgtcacggcggagttcacgcgccccgactcccgctgcccctgcgacggcga
cgagtacgactgcaagtacgccggcttccactgaccatcatgaacatcctgaacaaggccgcccccatgggccagaacgcgg
gcgtcggcggctggaacgacctggacaacctggaggtcggcgtcggcaacctgacggacgacgaggagaaggcgcacttctc
catgtgggccatggtgaagtcccccctgatcatcggcgcgaacgtgaacaacctgaaggcctcctcctactccatctactcccagg
cgtccgtcatcgccatcaaccaggactccaacggcatccccgccacgcgcgtaggcgctactacgtgtccgacacggacgagt
acggccagggcgagatccagatgtggtccggccccctggacaacggcgaccaggtcgtggcgctgctgaacggcggctccgtg
tcccgccccatgaacacgaccaggaggagatcttatcgactccaacctgggctccaagaagagacctccacctgggacatct
acgacctgtgggcgaaccgcgtcgacaactccacggcgtccgccatcctgggccgcaacaagaccgccaccggcatcctgtac
aacgccaccgagcagtcctacaaggacggcctgtccaagaacgacacccgcctgttcggccagaagatcggctccctgtcccc

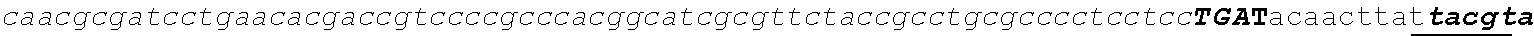

ttctgaccggcgctgatgtggcgcggacgccgtcgtactcatcagacatactcttgaggaattgaaccatctcgcttgctggcatgta
aacattggcgcaattaattgtgtgatgaagaaagggtggcacaagatggatcgcgaatgtacgagatcgacaacgatggtgattgttat
gaggggccaaacctggctcaatcttgtcgcatgtccggcgcaatgtgatccagcggcgtgactctcgcaacctggtagtgtgtgcgca
ccgggtcgctttgattaaaactgatcgcattgccatcccgtcaactcacaagcctactctagctcccattgcgcactcgggcgcccggct
cgatcaatgttctgagcggagggcgaagcgtcaggaaatcgtctcggcagctggaagcgcatggaatgcggagcggagatcgaat

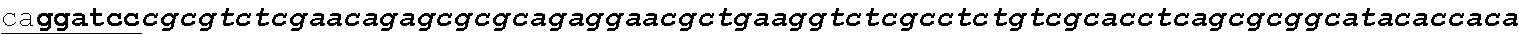

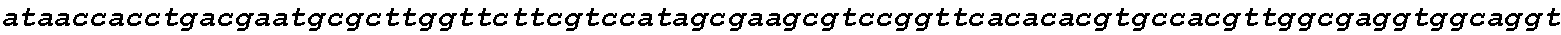

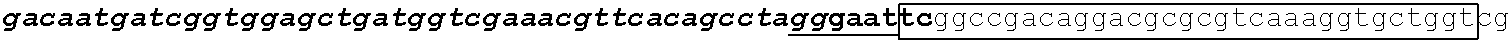

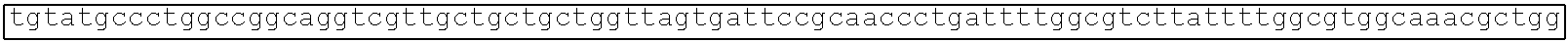

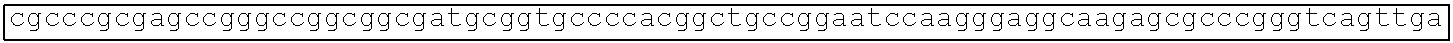

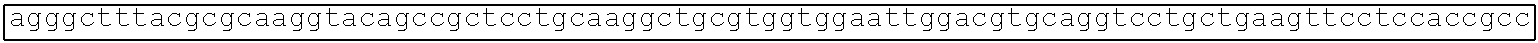

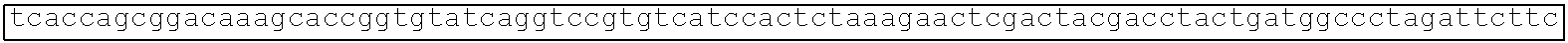

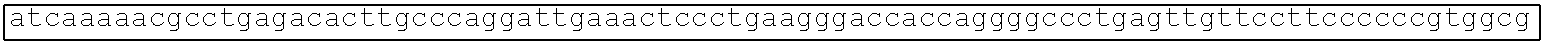

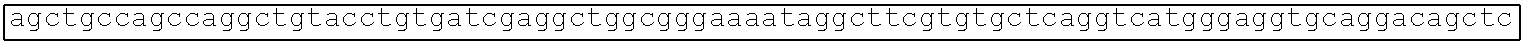

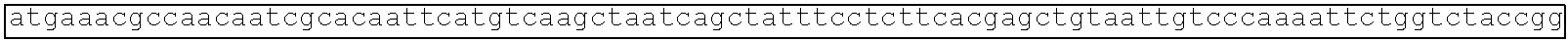

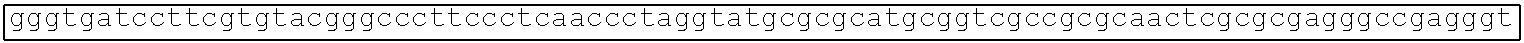

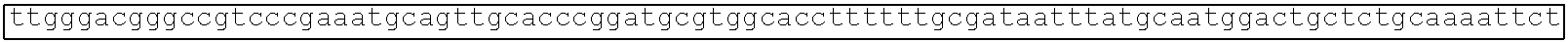

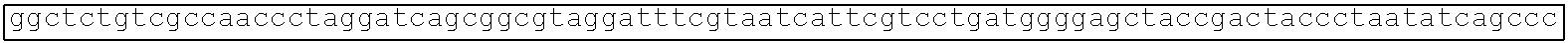

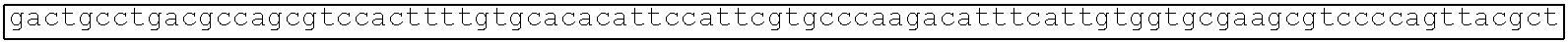

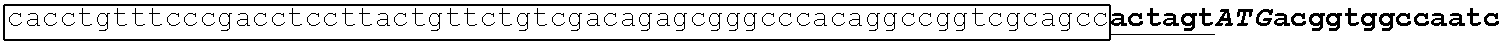

ccccggaagccccgttcgacagcgagggttcctcgctggcgcccgacaatgggtccagcaagcccaccaagctgagctccac
ccggtccttgctgtccatctcctaccgggagctctcgcgttccaagtgcgtgcaggggcgggggcaccttttgttggtgttgtttg
ggcgggcctcagcactggggtggaggaagaatgcgtgagtgtgcttgcacacctcggcggtttaagatgtaatgcgccaattt
cttgctgatgcattcctagacacaaagagtctctcattcgagtctcatcgcggttgtgcgctcctcactccgtgcagccagcagtc
gcggtcgttcacttcgcggggggtgccagggaggacggacgtttcggatgagctggagcgccgcatcctcgagtggcagggc
gatcgcgccatccacaggtcggttgggtgggaaagggggggcgttggggtcaggtcagaagtcgtgaagttacaggcctgca
tttgcacatcctgcgcgcgcctctggccgcttgtcttaagacccttgcactcgcttcctcatgaacccccatgaactccctcctgc
accccacagcgtgctggtggccaacaacggtctggcggcggtcaagttcatccggtcgatccggtcgtggtcgtacaagacgt
ttgggaacgagcgtgcggtgaagctgatcgcgatggcgacgcccgaggacatgcgcgcggacgcggagcacatccgcatgg
cggaccagtttgtggaggtccccggcggcaagaacgtgcagaactacgccaacgtgggcctgatcacctcggtggcggtgcg
caccggggtggacgcggtgcctgcagg.

In addition to pSZ5931 described above, constructs hijacking PmACCase1-2 promoter with PmAMT03 for transformation into S7708 or S8414 have also been constructed. These constructs are described as:

pSZ5932—PmACCase1-2::PmHXT1v2-ScarMEL1-PmPGK-BDNA:BDNA:PmAMT03::PmACCase1-2

pSZ6106—PmACCase1-1::PmLDH1v2p-AtTHIC(L337M)-PmHSP90-BDNA:PmAMT03::PmACCase1-1

pSZ6107—PmACCase1-2::PmLDH1v2p-AtTHIC(L337M)-PmHSP90-BDNA:PmAMT03::PmACCase1-2

pSZ5932 has the same vector backbone; selectable marker, promoters, and 3′ utr as pSZ5931, differing only in PmACCase flanks used for integration. While pSZ5931 is targeted to PmACCase1-1, pSZ5932 is targeted to PmACCase1-2 genomic locus. Nucleotide sequences of PmACCase1-2 5′ flank and PmACCase1-2 3′ flank and are shown below. Relevant restriction sites as underlined bold text are shown 5′-3′ respectively.

Nucleotide sequence of PmACCase 5′ flank contained
in plasmid pSZ5392 and pSZ6107 transformed into
S7708 and S8414, respectively:
(SEQ ID NO: 146)
Gattcatatcatcaaatttcgcatatgtttcacgagttgctcacaacatc
ggcaaatgcgttgttgttccctgtttttacaccttgccagggcctggtca
aagcttgacagtttgaccaaattcaggtggcctcatctctttcgcactga
tagacattgcagatttggaagacccagccagtacattacatgcacagcca
tttgctcctgcaccatgaacttgccacttttgtgcgccggtcgggggtga
tagctcggcagccgccgatcccaaaggtcccgcggcccaggggcacgaga
ccccccgacacgattaaatagccaaaatcagtcagaacggcacctccacc
ctacccgaatctgacaaggtcatcaaacgcgcgaaacaacggcgagggtg
cgttcgggaagcgcgcgtagttgacgcaagaagcctgggtcaggctggag
ggccgcgagaagatcgcttcctgccgagtctgcacccacgcctcgagcgc
accgtccgcgaacaaccaaccccttttcgcgagccctggcattctttcaa
ttgccaaggatgcacatgtgacacgtatagccattcggctttgtttgtgc
ctgcttgactcgcgccatttaattgttttgtgccggtgagccgggagtcg
gccactcgtctccgagccgcagtcccggcgccagtcccccggcctctgat
ctgggtccggaagggttggtataggagcagtctcggctatctgaagcccg
ttaccagacactttggccggctgattccaggcagccgtgtactcttgcgc
agtcggtacc.
Nucleotide sequence of PmACCase 3′ flank contained
in plasmid pSZ5392 and pSZ6107 transformed into
S7708 and S8414, respectively:
(SEQ ID NO: 147)
actagtATGacggtggccaatcccccggaagccccgttcgacagcgaggg
ttcctcgctggcgcccgacaatgggtccagcaagcccaccaagctgagct
ccacccggtccctgctgtccatctcctaccgggagctctcgcgttccaag
tgcgtacaggggcgagggcaccttttgttggtgttgtttgggcgggcctc
ggtactgggaggaggaggaatgcgtgcacacctctgcggttttagatgca
atgcgacaagtgcctgctgatgcattttctagacatgaagcatctcgtat
tcgagtctcaacgcgggtgtgcgctcctcactccgtgcagccagcagtcg
cggtcgttcacttcgcggggggtgccagggaggacggacgtttcggatga
gctggagcgccgcatcctcgagtggcagggcgatcgcgccatccacaggt
cggttgggtgggaaagggggagtaccggggtcaggtcagaagtcgtgcat
ttacaggcatgcatctgcacatcgtgcgcacgcgcacgtctttggccgct
tgtctcaagactcttgcactcgtttcctcatgcaccataatcaattccct
cccccctcgcaaactcacagcgtgctggtggccaacaacggtctggcggc
ggtcaagttcatccggtcgatccggtcgtggtcgtacaagacgtttggga
acgagcgcgcggtgaagctgattgcgatggcgacgcccgagggcatgcgc
gcggacgcggagcacatccgcatggcggaccagtttgtggaggtccccgg
cggcaagaacgtgcagaactacgccaacgtgggcctgatcacctcggtgg
cggtgcgcaccggggtggacgcggtgcctgcagg.

pSZ6106 is identical to pSZ5931, while pSZ6107 is identical to pSZ5932 except for the selectable marker module. While both pSZ5931 and pSZ5932 use S. carlbergensis MEL1 driven by PmHXT1v2 promoter and PmPGK as 3′ UTR as a selectable marker module, pSZ5073 and pSZ5074 uses Arabidopsis thaliana THiC driven by pmLDH1 promoter and PmHSP90 3′ UTR instead. Nucleotide sequence of the PmLDH1 promoter, AtThiC gene and PmHSP90 3′ UTR contained in pSZ6106 and pSZ6107 is shown below.

Nucleotide sequence of PmLDH1 promoter (boxed lowercase text), CpSAD transit
peptide (underlined lowercase text) and AtThiC-L337M (lowercase italic text) gene with and
PmHSP90 3' UTR (lowercase text) contained in pSZ6106 and pSZ6107 transformed into
S8414. Rcstriction sites in 5′ -3′ direction shown in bold underlined text are KpnI, NheI,
AscI, SnaBI and BamHI, respectively:
(SEQ ID NO: 148)

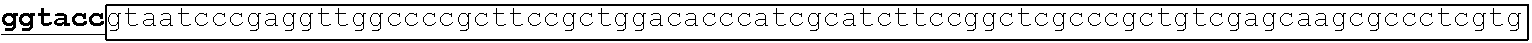

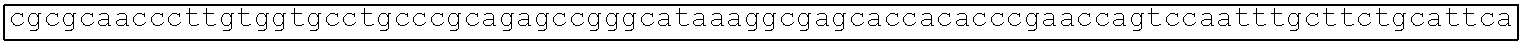

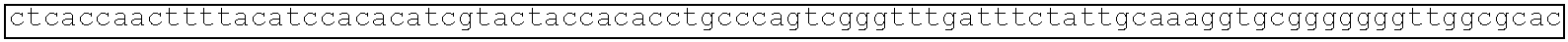

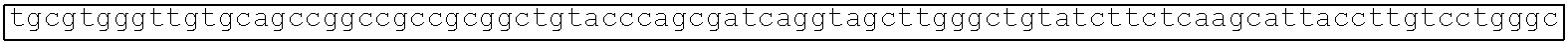

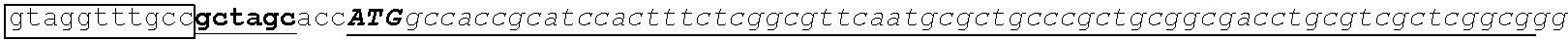

ctccgggccccggcgcccagcgaggcccctccccgtgcgcgggcgcgccgtccaggccgcggccacccgcttcaagaaggag
acgacgaccacccgcgccacgctgacgttcgacccccccacgaccaactccgagcgcgccaagcagcgcaagcacaccatc
gacccctcctcccccgacttccagcccatcccctccttcgaggagtgcttccccaagtccacgaaggagcacaaggaggtggtgc
acgaggagtccggccacgtcctgaaggtgcccttccgccgcgtgcacctgtccggcggcgagcccgccttcgacaactacgaca
cgtccggcccccagaacgtcaacgcccacatcggcctggcgaagctgcgcaaggagtggatcgaccgccgcgagaagctggg
cacgccccgctacacgcagatgtactacgcgaagcagggcatcatcacggaggagatgctactgcgcgacgcgcgagaag
ctggaccccgagttcgtccgctccgaggtcgcgcggggccgcgccatcatcccctccaacaagaagcacctggagctggagcc
catgatcgtgggccgcaagttcctggtgaaggtgaacgcgaacatcggcaactccgccgtggcctcctccatcgaggaggaggt
ctacaaggtgcagtgggccaccatgtggggcgccgacaccatcatggacctgtccacgggccgccacatccacgagacgcgcg
agtggatcctgcgcaactccgcggtccccgtgggcaccgtccccatctaccaggcgctggagaaggtggacggcatcgcggag
aacctgaactgggaggtgttccgcgagacgctgatcgagcaggccgagcagggcgtggactacttcacgatccacgcgggcgt
gctgctgcgctacatccccctgaccgccaagcgcatgacgggcatcgtgtcccgcggcggctccatccacgcgaagtggtgcctg
gcctaccacaaggagaacttcgcctacgagcactgggacgacatcctggacatctgcaaccagtacgacgtcgccctgtccatc
ggcgacggcctgcgccccggctccatctacgacgccaacgacacggcccagttcgccgagctgctgacccagggcgagctgac
gcgccgcgcgtgggagaaggacgtgcaggtgatgaacgagggccccggccacgtgcccatgcacaagatccccgagaacat
gcagaagcagctggagtggtgcaacgaggcgcccttctacaccctgggccccctgacgaccgacatcgcgcccggctacgacc
acatcacctccgccatcggcgcggccaacatcggcgccctgggcaccgccctgctgtgctacgtgacgcccaaggagcacctgg
gcctgcccaaccgcgacgacgtgaaggcgggcgtcatcgcctacaagatcgccgcccacgcggccgacctggccaagcagca
cccccacgcccaggcgtgggacgacgcgctgtccaaggcgcgcttcgagttccgctggatggaccagttcgcgctgtccctggac
cccatgacggcgatgtccttccacgacgagacgctgcccgcggacggcgcgaaggtcgcccacttctgctccatgtgcggcccc
aagttctgctccatgaagatcacggaggacatccgcaagtacgccgaggagaacggctacggctccgccgaggaggccatcc
gccagggcatggacgccatgtccgaggagttcaacatcgccaagaagacgatctccggcgagcagcacggcgaggtcggcg

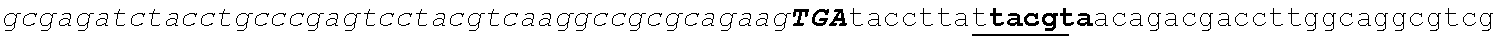

ggtagggaggtggtggtgatggcgtctcgatgccatcgcacgcatccaacgaccgtatacgcatcgtccaatgaccgtcggtgtcctc
tctgcctccgttttgtgagatgtctcaggcttggtgcatcctcgggtggccagccacgttgcgcgtcgtgctgcttgcctctcttgcgcctc
tgtggtactggaaaatatcatcgaggcccgatattgctcccataccatccgctacatcttgaaagcaaacgacaaacgaagcagcaa
gcaaagagcacgaggacggtgaacaagtctgtcacctgtatacatctatttccccgcgggtgcacctactctctctcctgccccggcag
agtcagctgccttacgtgacggatcc.

To determine their impact on fatty acid profiles, the constructs described above were transformed independently into S7708 (pSZ5391; D4383 and pSZ5392; D4384) or S8414 (pSZ6106; D5073 and pSZ6107; D5074). Primary transformants were clonally purified and grown under standard lipid production conditions at pH7.0. pH 7 was chosen to allow for maximal expression of PmACCase1-1 or PmACCase1-2 genes being upregulated by our pH regulated AMT03 (Ammonium transporter 03) promoter. The resulting profiles from a set of representative clones arising from transformations with pSZ5391 (D4383), pSZ5392 (D4384), pSZ6106 (D5073) and pSZ6107 (D5074) and shown in Tables 106-110 below.

TABLE 106
Fatty acid profiles of representative S7708 and strains transformed
with D4383 (pSZ5391 - PmAccase1-1 upregulation).

| | Fatty acid profile | | | | | |
|---|---|---|---|---|---|---|
| Sample ID | C18:0 | C18:1 | C18:2 | C18:3α | C20:1 | C22:1 |
| S7708; pH 7 | 1.77 | 50.47 | 7.93 | 0.67 | 2.97 | 1.53 |
| S7708; T1215; | 1.02 | 32.85 | 14.68 | 1.87 | 4.44 | 7.61 |
| D4383-1; | | | | | | |
| pH 7 | | | | | | |
| S7708; T1215; | 1.64 | 51.32 | 8.34 | 0.73 | 3.01 | 1.70 |
| D4383-10; | | | | | | |
| pH 7 | | | | | | |
| S7708; T1215; | 1.47 | 41.77 | 9.57 | 1.10 | 2.48 | 1.46 |
| D4383-6; | | | | | | |
| pH 7 | | | | | | |
| S7708; T1215; | 1.61 | 51.17 | 8.01 | 0.70 | 2.43 | 1.35 |
| D4383-3; | | | | | | |
| pH 7 | | | | | | |
| S7708; T1215; | 1.61 | 50.99 | 8.33 | 0.65 | 2.36 | 1.33 |
| D4383-2; | | | | | | |
| pH 7 | | | | | | |

TABLE 107
Primary Fatty acid profiles of representative S7708 and strains
transformed with D4383 (pSZ5392 - PmAccase1-2 upregulation)

| | Fatty acid profile | | | | | |
|---|---|---|---|---|---|---|
| Sample ID | C18:0 | C18:1 | C18:2 | C18:3α | C20:1 | C22:1 |
| S7708; pH 7 | 1.74 | 50.39 | 7.93 | 0.68 | 3.02 | 1.54 |
| S7708; T1215; | 1.08 | 34.60 | 14.27 | 1.69 | 4.28 | 6.71 |
| D4384-1; | | | | | | |
| pH 7 | | | | | | |
| S7708; T1215; | 1.60 | 51.06 | 8.15 | 0.67 | 3.02 | 1.70 |
| D4384-7; | | | | | | |
| pH 7 | | | | | | |
| S7708; T1215; | 1.59 | 50.49 | 8.33 | 0.67 | 3.02 | 1.60 |
| D4384-2; | | | | | | |
| pH 7 | | | | | | |
| S7708; T1215; | 1.72 | 51.48 | 7.96 | 0.70 | 2.78 | 1.51 |
| D4384-4; | | | | | | |
| pH 7 | | | | | | |
| S7708; T1215; | 1.63 | 51.56 | 7.98 | 0.64 | 2.95 | 1.50 |
| D4384-5; | | | | | | |
| pH 7 | | | | | | |

D4383-1 (7.61% C22:1) and D4384-1 (6.71% C22:1) showed more than a 3 fold increase in C22:1 levels over the parent S7708. Both the strains were subsequently found to have stable phenotypes. D5073-45 (13.61% C22:1) and D5074-15 (9.62% C22:1) showed 2.95 and 2.11 fold increases in C22:1 levels over the parent S8414 (4.60% C22:1). Selected S8414 lines transformed with either D5073 or D5074 were run at pH5 and pH7 to regulate the PmAMT03 driven PmACCase1-1 or PmACCase1-2 gene expression (table 110). Shutting down the PmACCAse1-1 or PmACCase1-2 at pH5.0 led to near parental levels of C22:1 in all the selected lines, confirming the positive impact of PmACCase upregulation on very long chain fatty acid biosynthesis in our host. These results conclusively demonstrate that increasing the Malonyl-CoA via upregulation of PmACCase1-1 or PmACCase1-2 results in significant increase in the very long chain fatty acid biosynthesis in P. moriformis expressing a heterologous fatty acid elongase. pH5/pH7 experiments cannot be performed on S7708 derived transformants since the heterologous LaFAE in parent S7708 is also driven by PmAMT03 and running the lines at pH5.0 would lead to shutting off of the elongase as well.

TABLE 108
Fatty acid profiles of representative S8414 and strains transformed
with D5073 (pSZ6106 - PmAccase1-1 upregulation).

| | Fatty acid profile | | | | | |
|---|---|---|---|---|---|---|
| Sample ID | C18:0 | C18:1 | C18:2 | C18:3α | C20:1 | C22:1 |
| S8414 | 1.36 | 38.95 | 11.90 | 0.88 | 7.50 | 4.60 |
| S8414; T1435; | 1.16 | 24.00 | 13.24 | 2.09 | 8.42 | 13.61 |
| D5073-45 | | | | | | |
| S8414; T1435; | 0.90 | 29.65 | 16.64 | 1.05 | 9.09 | 9.63 |
| D5073-8 | | | | | | |
| S8414; T1435; | 0.83 | 29.14 | 15.64 | 1.42 | 7.25 | 9.48 |
| D5073-24 | | | | | | |
| S8414; T1435; | 0.88 | 35.26 | 16.57 | 0.47 | 11.02 | 9.26 |
| D5073-44 | | | | | | |
| S8414; T1435; | 1.02 | 35.12 | 13.82 | 1.06 | 7.97 | 7.31 |
| D5073-21 | | | | | | |

TABLE 109
Fatty acid profiles of representative S8414 and strains transformed
with D5074 (pSZ6107 - PmAccase1-2 upregulation).

| | Fatty acid profile | | | | | |
|---|---|---|---|---|---|---|
| Sample ID | C18:0 | C18:1 | C18:2 | C18:3α | C20:1 | C22:1 |
| S8414 | 1.36 | 38.95 | 11.90 | 0.88 | 7.50 | 4.60 |
| S8414; T1435; | 1.22 | 36.19 | 12.60 | 0.86 | 9.56 | 9.62 |
| D5074-15 | | | | | | |
| S8414; T1435; | 1.11 | 33.08 | 13.33 | 1.11 | 8.51 | 8.12 |
| D5074-1 | | | | | | |
| S8414; T1435; | 1.06 | 32.72 | 13.40 | 1.16 | 7.84 | 7.75 |
| D5074-9 | | | | | | |
| S8414; T1435; | 1.12 | 34.13 | 13.01 | 1.01 | 8.49 | 7.53 |
| D5074-2 | | | | | | |
| S8414; T1435; | 0.86 | 31.63 | 13.51 | 0.80 | 5.90 | 6.95 |
| D5074-10 | | | | | | |

TABLE 110
Fatty acid profiles of selected S8414 strains transformed
with D5073 and D5074 run at pH 5 and pH 7.

| | Fatty acid profile | | | | | |
|---|---|---|---|---|---|---|
| Sample ID | C18:0 | C18:1 | C18:2 | C18:3 a | C20:1 | C22:1 |
| S7485; pH 5 | 3.84 | 50.91 | 5.41 | 0.49 | 0.07 | 0.00 |
| S7485; pH 7 | 4.24 | 45.95 | 5.56 | 0.61 | 0.05 | 0.00 |
| S8414; pH 5 | 1.62 | 47.70 | 9.36 | 0.59 | 6.36 | 2.57 |
| S8414; pH 7 | 1.40 | 38.78 | 11.50 | 0.84 | 7.79 | 4.75 |
| S8414; T1435; | 0.93 | 43.04 | 13.65 | 0.97 | 6.33 | 3.18 |
| D5073-8; | | | | | | |
| pH 5 | | | | | | |
| S8414; T1435; | 0.90 | 30.19 | 16.45 | 1.10 | 9.11 | 9.46 |
| D5073-8; | | | | | | |
| pH 7 | | | | | | |
| S8414; T1435; | 1.32 | 34.54 | 10.86 | 1.44 | 8.74 | 6.36 |
| D5073-45; | | | | | | |
| pH 5 | | | | | | |
| S8414; T1435; | 1.22 | 25.44 | 12.81 | 1.99 | 9.02 | 13.08 |
| D5073-45; | | | | | | |
| pH 7 | | | | | | |
| S8414; T1435; | 1.37 | 44.32 | 10.57 | 0.76 | 7.40 | 3.76 |
| D5074-1; | | | | | | |
| pH 5 | | | | | | |
| S8414; T1435; | 1.16 | 34.05 | 12.92 | 1.09 | 8.56 | 7.19 |
| D5074-1; | | | | | | |
| pH 7 | | | | | | |
| S8414; T1435; | 1.32 | 46.03 | 9.79 | 0.62 | 8.68 | 4.34 |
| D5074-15; | | | | | | |
| pH 5 | | | | | | |
| S8414; T1435; | 1.25 | 36.95 | 12.58 | 0.88 | 9.58 | 8.95 |
| D5074-15; | | | | | | |
| pH 7 | | | | | | |

### Example 20

### Expression of 3-Ketoacyl-CoA Reductase (KCR), Enoyl-CoA Reductase (ECR), Hydroxyacyl-CoA Hydratase (HACD), and Acetyl-CoA Carboxylase (ACCase)

In this example, we report the outcome of co-expression of Ketoacyl-CoA Reductase (KCR) and Enoyl-CoA Reductase (ECR) or Hydroxyacyl-CoA Dehydratase (HACD) enzymes involved in very long chain fatty acid biosynthesis, in P. moriformis (UTEX 1435). Simultaneously we also upregulated the endogenous cytosolic homomeric Acetyl-CoA carboxylase (ACCase) by hijacking the promoter of either PmACCase1-1 or PmACCase1-2 and replacing it with PmAMT03 promoter. Our results demonstrate that combining the heterologous KCR and ECR or HACD activities with up-regulated endogenous ACCase activity in S8414 and S8242 results in a significant increase (more than 4-fold) in C22:1 levels in the resulting transgenic lines. S8414 is described above. S8242 was generated by expressing Limnanthes douglasii LPAAT in S7708 as discussed in Example 10.

Crambe abyssinica fatty acid elongase (CrhFAE) is a very active FAE in Prototheca. We codon optimized and synthesized nucleic acids encoding CrhKCR, CrhHACD and CrhECR and expressed them in S7211 (CrhFAE strain) and S7708 (Lunaria annua FAE strain). The codon-optimized genes were cloned into appropriate expression vectors and transformed into both S7708 and S7211. Expression of each of the partner genes in both S7708 and S7211 resulted in improved VLCFA biosynthesis. The increase in C22:1 was between 1.2 to 1.9 fold over the parent strains. Further, we disclosed above that we increased the availability of malonyl-CoA by upregulation of endogenous PmACCase and this led to significant increases the long chain fatty acid biosynthesis in a strain already expressing a FAE (3 or more fold increase in C22:1 in S7708 and S8414 backgrounds). To further increase VLCFA biosynthesis we performed the following: Combine KCR, ECR and HACD activities with upregulated PmACCase in a strain already expressing a FAE (S8414) to maximize the VLCFA biosynthesis; and Expression of above activities in a strain like S8242 further increased VLCFA biosynthesis since in addition to a FAE activity, S8242 also expresses an erucic acid preferring LPAAT from Limnanthes douglasii (LimdLPAAT).

We made constructs to co-express CrhKCR (driven by either PmACPP1 or PmG3PDH promoter) along with CrhECR or CrhHACD (driven by PmG3PDH or PmACPP1 promoters) in S8414 (3.3% C22:1; PmSAD2-2v2-CrhFAE-PmHSP90) and S8242 (5-7% C22:1; PmAMT03-LaFAE-CvNR and PmSAD2-2v2-LimdLPAAT-CvNR) strains. The constructs were targeted to PmACCase1-1 or PmACCase1-2 loci while simultaneously hijacking the promoter of the endogenous PmACCase1-1 or PmACCAse1-2 with the pH regulatable Ammonia transport 3 (PmAMT03) promoter. The “promoter hijack” was accomplished by inserting the PmAMT03 promoter between the endogenous PmACCCase1-1 or PmACCase 1-2 promoter and the initiation codon of the PmACCase1-1 or PmACCase1-2 gene in both S8414 and S8242.

Construct Used for the Coexpression of ECR and KCR while Simultaneously Up Regulating P. Moriformis Acetyl-CoA Carboxylase (PmACCase) in Erucic Strains S8414 and S8242—[pSZpSZ6114)

S8414 and S8242 strains were transformed with the construct pSZ6114, which expresses a mutant version (L337M) of Arabidopsis thaliana ThiC gene driven by PmLDH1v2 promoter (allowing for their selection and growth on medium without thiamine), CrhECR driven by PmACPP1 promoter, CrhKCR driven by PmG3PDH promoter and endogenous P. morformis ACCase driven by PmAMT03 promoter (promoter hijack). Construct pSZ5391 is described above. Construct pSZ6114 for expression in S8414 and S8242 can be written as:
PmACCase 1-1 PmLDH1v2p-AtTHIC(L337M):PmHSP90:BDNA:PmACPP1-CrhECR-CvNR:PmG3 PDH-CrhKCRCvNR:PmAMT03::PmACCase1-1.

The sequence of transforming DNA (pSZ6114) is provided below. Relevant restriction sites in the construct are indicated in lowercase, underlined bold, and are from 5′-3′ NdeI, KpnI, NcoI, SnaBI, BamHI, EcoRI, SpeI, XhoI, XbaI, SpeI, XhoI, EcoRV, SpeI and SbfI respectively. NdeI and AseI sites delimit the 5′ and 3′ ends of the transforming DNA. Bold, lowercase sequences represent genomic DNA from S3150 that permit targeted integration at the ACCase locus via homologous recombination. Proceeding in the 5′ to 3′ direction, the endogenous P. moriformis lactate dehydrogenase (LDH) promoter driving the expression of the Arabidopsis thaliana THiC is indicated by lowercase, boxed text. Uppercase italics indicate the initiator ATG and terminator TGA for AtThiC, while the coding region is indicated with lowercase italics. The P. moriformis heat shock protein 90 (HSP90) gene 3′ UTR is indicated by lowercase underlined text followed by buffer/spacer DNA sequence indicated by lowercase bold italic text Immediately following the buffer DNA is an endogenous Acyl Carrier protein (ACPP1) promoter of P. moriformis, indicated by boxed lowercase text. Uppercase italics indicate the initiator ATG and terminator TGA for C. abyssinica enoyl-CoA reductase (CrhECR) gene while the coding region is indicated with lowercase italics. The Chlorella vulgaris nitrate reductase (CvNR) gene 3′ UTR is indicated by lowercase underlined text immediately followed by endogenous G3PDH promoter indicated by lower case boxed text. Uppercase italics indicate the initiator ATG and terminator TGA for C. abyssinica Ketoacyl-CoA reductase (CrhKCR) gene while the coding region is indicated with lowercase italics. The Chlorella vulgaris nitrate reductase (CvNR) gene 3′ UTR is indicated by lowercase underlined text Immediately following the CvNR 3 UTR is an endogenous AMT03 promoter of P. moriformis, indicated by boxed lowercase text followed by the PmACCCase1-1 genomic region indicated by bold, lowercase text. Uppercase, bold italics indicate the Initiator ATG of the endogenous PmACCase1-1 gene targeted for upregulation by preceding PmAMT03 promoter. The final construct was sequenced to ensure correct reading frames and targeting sequences.

Nucleotide sequence of transforming DNA contained in plasmid pSZ6114
transformed into S8414 and S8242:
(SEQ ID NO: 149)
catatgtttcacgcgttgctcacaacaccggcaaatgcgttgttgttccctgtttttacaccttgccagagcctggtcaaagcttg
acagtttgaccaaattcaggtggcctcatctctctcgcactgatagacattgcagatttggaagacccagtcagtacactacatg
cacagccgtttgctcctgcgccatgaacttgccacttttgtgcgccggtcgggggtgatagctcggcagccgccgatcccaaag
gtcccgcggcccaggggcacgagaacccccgacacgattaaatagccaaaatcagttagaacggcacctccaccctacccg
aatctgacagggtcatcaagcgcgcgaaacaacggcgagggtgcgttcgggaagcgcgcgtagttgacgcaagaagcctgg
gtcaggctgggagggccgcgagaagatcgcttcctgccgagtctgcacccacgcctcgagcgcaccgtccgcgaacaacca
acccctttgcgcgagccctgacattctttcaattgccaaggatgcacatgtgacacgtatagccattcggctttgtttgtgcctgct
tgactcgcgtcatttaattgatttgtgccggtgagccgggagtcggccactcgtctccgagccgcagtcccggcgccagtcccc
cggcctctgatctgggtccggaagggttggtataggagcggtctcggctatctgaagcccattacccgacactttggccggctg

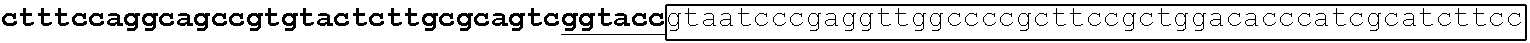

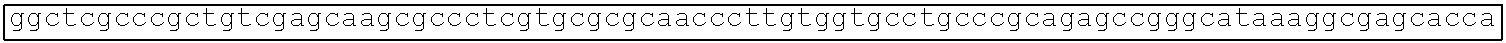

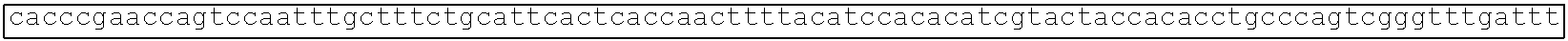

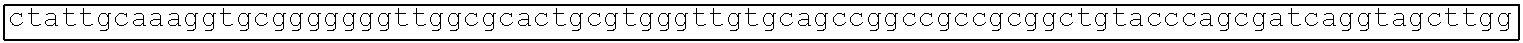

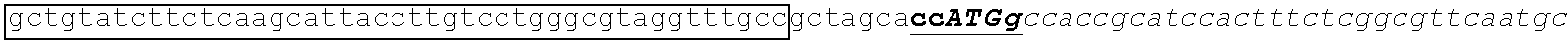

ccgctgcggcgacctgcgtcgctcggcgggctccgggccccggcgcccagcgaggcccctccccgtgcgcgggcgcgccgtcc
aggccgcggccacccgcttcaagaaggagacgacgaccacccgcgccacgctgacgttcgacccccccacgaccaactccga
gcgcgccaagcagcgcaagcacaccatcgacccctcctcccccgacttccagcccatcccctccttcgaggagtgcttccccaag
tccacgaaggagcacaaggaggtggtgcacgaggagtccggccacgtcctgaaggtgcccttccgccgcgtgcacctgtccgg
cggcgagcccgccttcgacaactacgacacgtccggcccccagaacgtcaacgcccacatcggcctggcgaagctgcgcaag
gagtggatcgaccgccgcgagaagctgggcacgccccgctacacgcagatgtactacgcgaagcagggcatcatcacggagg
agatgctgtactgcgcgacgcgcgagaagctggaccccgagttcgtccgctccgaggtcgcgcggggccgcgccatcatcccct
ccaacaagaagcacctggagctggagcccatgatcgtgggccgcaagttcctggtgaaggtgaacgcgaacatcggcaactcc
gccgtggcctcctccatcgaggaggaggtctacaaggtgcagtgggccaccatgtggggcgccgacaccatcatggacctgtcc
acgggccgccacatccacgagacgcgcgagtggatcctgcgcaactccgcggtccccgtgggcaccgtccccatctaccaggc
gctggagaaggtggacggcatcgcggagaacctgaactgggaggtgttccgcgagacgctgatcgagcaggccgagcaggg
cgtggactacttcacgatccacgcgggcgtgctgctgcgctacatccccctgaccgccaagcgcatgacgggcatcgtgtcccgc
ggcggctccatccacgcgaagtggtgcctggcctaccacaaggagaacttcgcctacgagcactgggacgacatcctggacatc
tgcaaccagtacgacgtcgccctgtccatcggcgacggcctgcgccccggctccatctacgacgccaacgacacggcccagttc
gccgagctgctgacccagggcgagctgacgcgccgcgcgtgggagaaggacgtgcaggtgatgaacgagggccccggccac
gtgcccatgcacaagatccccgagaacatgcagaagcagctggagtggtgcaacgaggcgccatctacaccctgggccccct
gacgaccgacatcgcgcccggctacgaccacatcacctccgccatcggcgcggccaacatcggcgccctgggcaccgccctgc
tgtgctacgtgacgcccaaggagcacctgggcctgcccaaccgcgacgacgtgaaggcgggcgtcatcgcctacaagatcgcc
gcccacgcggccgacctggccaagcagcacccccacgcccaggcgtgggacgacgcgctgtccaaggcgcgcttcgagttcc
gctggatggaccagttcgcgctgtccctggaccccatgacggcgatgtccttccacgacgagacgctgcccgcggacggcgcga
aggtcgcccacttctgctccatgtgcggccccaagttctgctccatgaagatcacggaggacatccgcaagtacgccgaggaga
acggctacggctccgccgaggaggccatccgccagggcatggacgccatgtccgaggagttcaacatcgccaagaagacgat

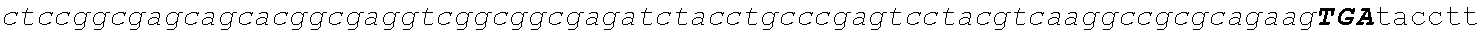

attacgtaacagacgaccaggcaggcgtcgggtagggaggtggtggtgatggcgtctcgatgccatcgcacgcatccaacgaccg
tatacgcatcgtccaatgaccgtcggtgtcctctctgcctccgttttgtgagatgtctcaggcttggtgcatcctcgggtggccagccacg
ttgcgcgtcgtgctgcttgcctctcttgcgcctctgtggtactggaaaatatcatcgaggcccgtttttttgctcccatttcctttccgctacat
cttgaaagcaaacgacaaacgaagcagcaagcaaagagcacgaggacggtgaacaagtctgtcacctgtatacatctatttccccgc
gggtgcacctactctctctcctgccccggcagagtcagctgccttacgtgacggatcccgcgtctcgaacagagcgcgcagagga
acgctgaaggtdcgcctagtcgcacctcagcmgcatacaccacaataaccacctgacgaatgcgcttggttcttcgtcca
ttagcgaagcgtccggttcacacacgtgccacgttggcgaggtggcaggtgacaatgatcggtggagctgatggtcgaaacg

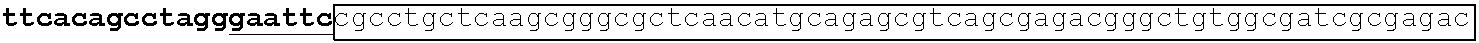

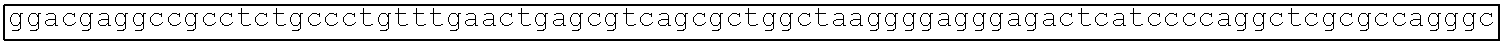

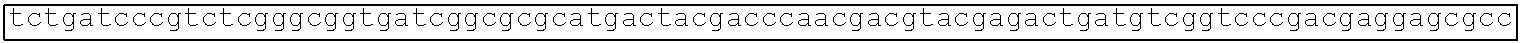

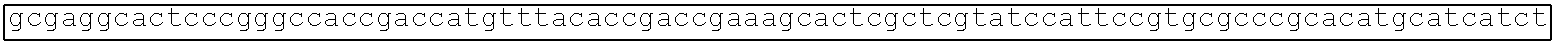

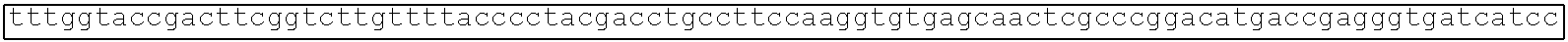

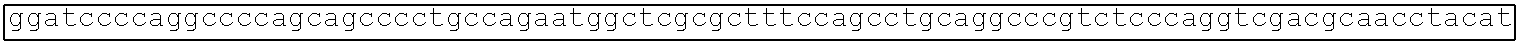

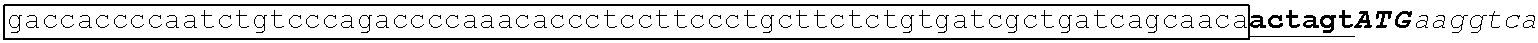

cggtggtgagcaggtccggcagggaggtgacaaggcccccaggacctgccggactccgccacggtcgctgacctccaggag
gccttccacaagcgcgaagaagttttatcccagccgccagcggctgaccagccggtggcccccggaccaaggacaagcc
ggtggtgctgaactcgaagaagagcctcaaggagtactgcgacggtaacaccgactcgctcacggtggtgtttaaggacttggg
cgcgcaggtacctaccgcaccagttcttatcgagtacctgggccccctgctgatctaccccgtatctactacttccagtctataag
tacctgggctacggcgaggaccgcgtcatccacccggtgcagacgtatgccatgtactactggtgatccactactttaagcgcatt
atggagacgttcttcgtgcaccgatcagccacgccacctcgcccatcggtaacgtatccgcaactmcctactactggacgttc
ggcgcctacatcgcttactacgtgaaccaccccctgtacacccccgtgagcgacttgcagatgaagatcggcttcgggttcggcct
cgtgtttcaggtggcgaacttctactgccacatcctgctgaagaatctgcgcgacccgaacggcagcggcggttaccagatcccg
cgcggcttcctgttcaacatcgtcacgtgcgcgaactacaccacggagatctaccagtggctcggattaacatcgccacgcagac
catcgccggctacgtgttcctcgcggtggccgccagattatgaccaactgggccacggcaagcactcgcggaccggaagatct

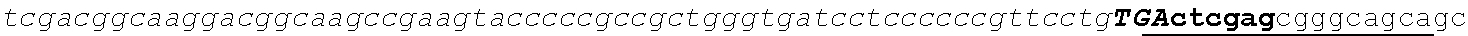

agctcggatagtatcgacacactctggacgctggtcgtgtgatggactgagccgccacacttgctgccttgacctgtgaatatccctgc
cgcattatcaaacagcctcagtgtgatgatcagtgtgtacgcgcattgcgagagctagctgcttgtgctatttgcgaataccaccccca
gcatcccatccctcgatcatatcgcttgcatcccaaccgcaacttatctacgctgtcctgctatccctcagcgctgctcctgctcctgctc
actgcccctcgcacagccttggtttgggctccgcctgtattctcctggtactgcaacctgtaaaccagcactgcaatgctgatgcacggg

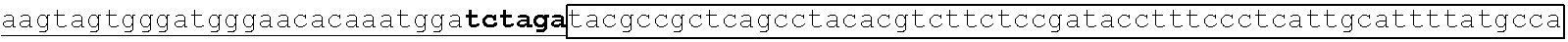

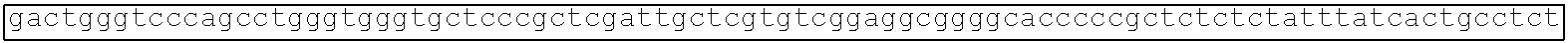

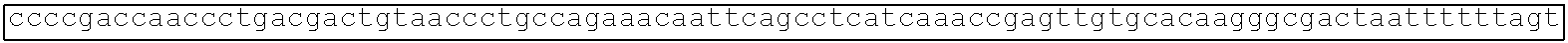

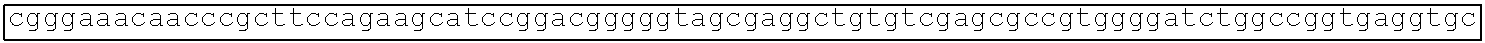

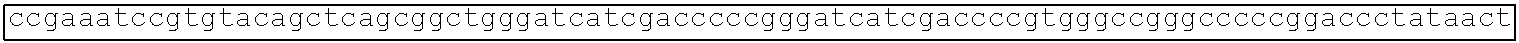

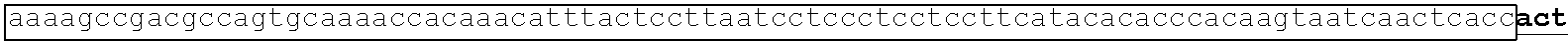

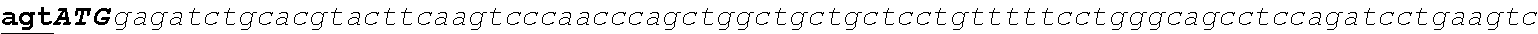

gacgttctccctcctgaagagcctgtacatctacttcctgcgccccggcaagaacctccgccgctacgggtcctgggccattatcac
cggcccgaccgacggcatcggcaaggcctttgcgttccagaggcccacaagggcctgaacctggtgctggtggcgcgcaaccc
ggacaagagaaggacgtaccgacagcatcaggtccaagcatagcaacgtgcagatcaagacggtgatcatggactttagcg
gcgacgttgacgacggcgtccgccgcatcaaggagaccatcgaggggctggaggtgggcatcctgatcaacaatgccggcatg
tcctacccgtacgcgaagtactttcacgaggtcgacgaggagctcgtcaacggcctcatcaaaatcaacgtcgagggcacgacc
aaggtgacccaggccgtgctgccgggcatgctggagcgcaagcgcggcgccatcgtcaacatgggcagcggcgcggccgccc
tgatcccgtcgtaccccttctacagcgtgtatgccggcgcgaagacgtacgtggaccagttcacccggtgcctgcacgtcgagtac
aagaagagcggcattgacgtccagtgccaggtcccgctctacgtggccacgaagatgacgaagatccgccgcgcctccttcctg
gtcgcctcccccgagggctacgccaaggccgccctgcggttcgtggggtacgaggcccggtgcaccccctactggccgcacgcc
ctgatgggctacgtcgtctccgccctgccccagtccgtgttcgagtccttcaacatcaagcgctgcctgcagatccgcaagaaggg

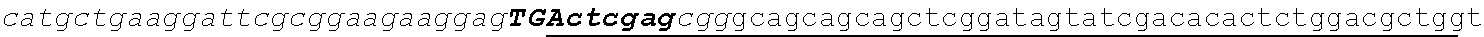

cgtgtgatggactgagccgccacacagctgccagacctgtgaatatccctgccgcttttatcaaacagcctcagtgtgatgatcagtg
tgtacgcgcttttgcgagttgctagctgcttgtgctatttgcgaataccacccccagcatccccttccctcgtttcatatcgcttgcatccca
accgcaacttatctacgctgtcctgctatccctcagcgctgctcctgctcctgctcactgcccctcgcacagccaggtagggctccgcc
tgtaactcctggtactgcaacctgtaaaccagcactgcaatgctgatgcacgggaagtagtgggatgggaacacaaatggagatatc

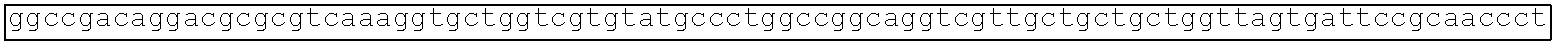

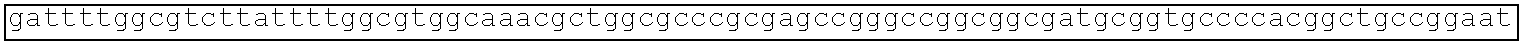

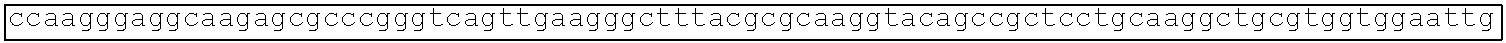

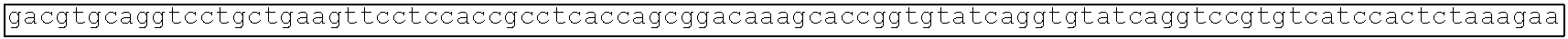

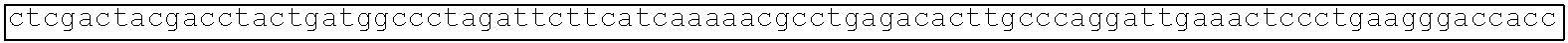

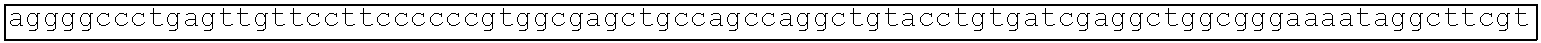

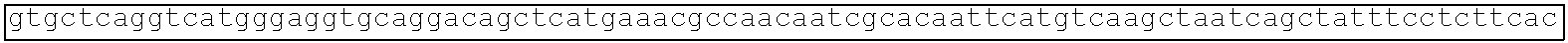

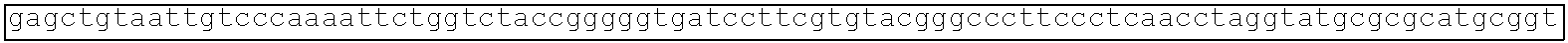

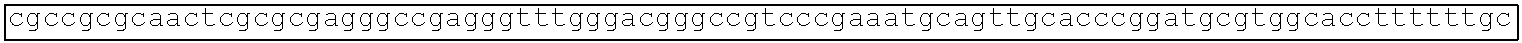

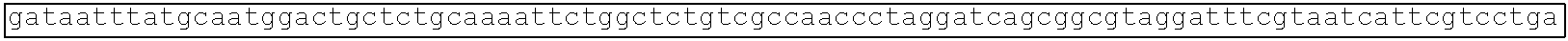

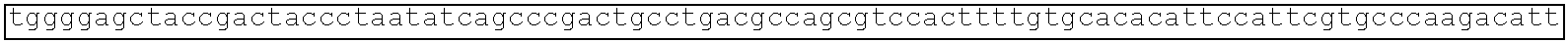

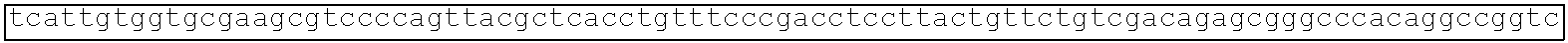

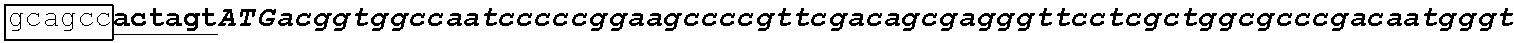

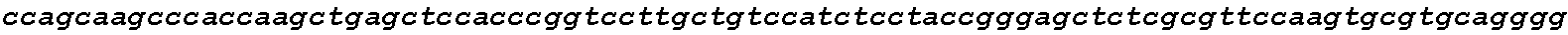

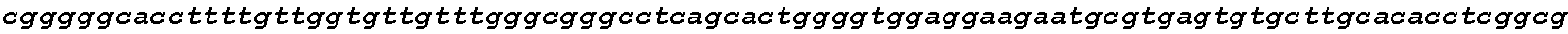

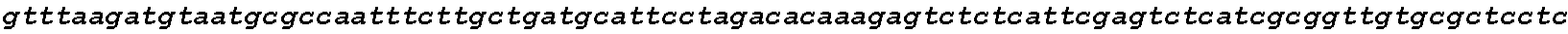

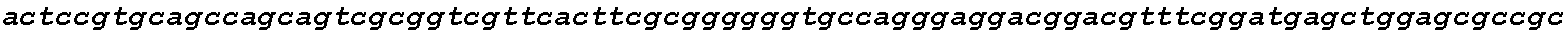

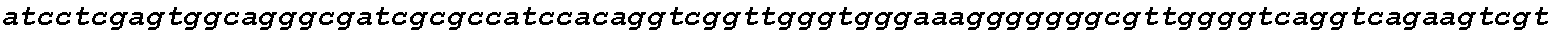

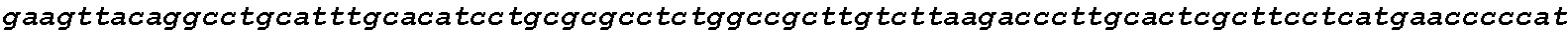

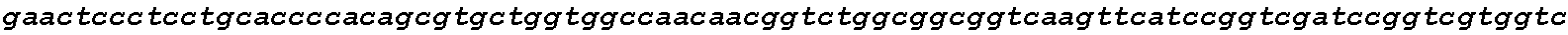

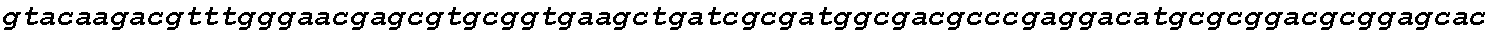

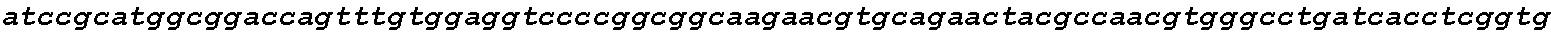

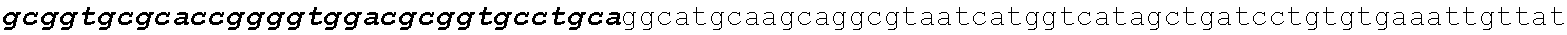

ccgctcacaaaccacacaacatacgagccggaagcataaagtgtaaagcctggggtgcctaatgagtgagctaactcacattaat.

In addition to C. abyssinica ECR and C. abyssinica KCR genes targeted at PmACCase1-1 locus while simultaneously upregulating the endogenous PmACCase1-1 gene (pSZ6114), several other constructs were designed for transformation into S8414 and S8242. These constructs can be described as:
pSZ6115-PmACCase1-1::PmLDH1 v2p-AtTHIC(L337M)-PmHSP90:BDNA::PmACPP1-CrhHACD-CvNR:PmG3PDH-CrhKCR-CvNR: PmAMT03::PmACCase1-1
pSZ6116-PmACCase1-1::PmLDH1 v2p-AtTHIC(L337M)-PmHSP90;BDNA::PmG3PDH-CrhECR-CvNR:PmACPP1-CrhKCR-CvNR:PmAMT03::PmACCase1-1
pSZ6117-PmACCase1-1::PmLDH1 v2p-AtTHIC(L337M)-PmHSP90:BDNA::PmG3PDH-CrhHACD-CvNR: PmACPP1-CrhKCR-CvNR: PmAMT03::PmACCase1-1
pSZ6118-PmACCase1-2::PmLDH1 v2p-AtTHIC(L337M):PmHSP90:BDNA:PmACPP1-CrhECR-CvNR:PmG3PDH-CrhKCR-CvNR: PmAMT03::PmACCase1-2
pSZ6119-PmACCase1-2::PmLDH1 v2p-AtTHIC(L337M)-PmHSP90:BDNA::PmACPP1-CrhHACD-CvNR: PmG3PDH-CrhKCR-CvNR: PmAMT03::PmACCase1-2
pSZ6120-PmACCase1-2::PmLDH1 v2p-AtTHIC(L337M)-PmHSP90:BDNA::PmG3PDH-CrhHACD-CvNR: PmACPP1 CrhKCR-CvNR: PmAMT03::PmACCase1-2

pSZ6115 is similar to pSZ6114 in every respect except the gene driven by PmACPP1 promoter. In pSZ6115 PmACPP1 promoter drives the expression of CrhHACD gene while in pSZ6114 it drives the expression of CrhECR. The nucleotide sequence of CrhHACD is shown below. pSZ6116 differs from pSZ6114 in that CrhECR is driven by PmG3PDH and CrhKCR is driven by PmACPP1 promoters while it is the opposite in pSZ6114 Similarly pSZ6118 is similar to pSZ6116 except that CrhHACD is driven by PmG3PDH and CrhKCR is driven by pmACPP1 promoters while it is opposite in pSZ6115. pSZ6118, pSZ6119 and pSZ6120 are same as pSZ6114, pSZ6115 and pSZ6117 respectively except that the former constructs are targeted to PmACCase1-2 locus while the latter ones are targeted to PmACCase1-1 locus. The PmACCase1-2 5 flank and PmACCAse1-2 3′ flank sequences used for targeting in pSZ6118, pSZ6119 and pSZ6120 are shown below. The initiator ATG of the endogenous PmACCase1-2 being upregulated by PmAMT03 is indicated in capital bold and italic letters. Relevant restriction sites as underlined bold text are shown 5′-3′ respectively.

Nucleotide sequence of CrhHACD gene in pSS6115, pSZ6117, pSZ6119 and
pSZ61120:
(SEQ ID NO: 150)

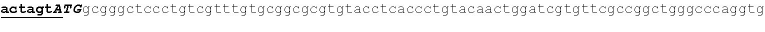

ctgtactttgccgtcaagacgctcaaggagtccggccacgagaacgtgtacgacgccgtggagaagcccctccagctggcgcaaac
cgccgcggtcctggagatcctccacggcctggtcggcctcgtcaggagcccggtctcggccaccctgccgcagatcgggagccgc
ctctttctgacctggggcattctgtattccttcccggaggtccagagccactcctggtgacctccctcgtgatcagctggtcgatcacgg
aaatcatccgctacagcttcttcggcctgaaggaggcgctgggcttcgcgcccagctggcacctgtggctccgctattcgagattctg
gtgctctaccccaccggcatcacctccgaggtcggcctcatctacctggccctgccgcacatcaagacgtcggagatgtactccgtcc
gcatgcccaacaccttgaaccttccccgactttttctacgccacgattctcgtcctcgcgatctacgtccccggttcgccccacatgtacc

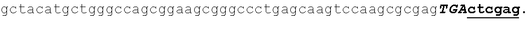

Nucleotide sequence of PmACCase 5′ flank contained in plasmids pSZ6118,
pSZ6119 and pSZ6120 respectively:
(SEQ ID NO: 151)
Gattcatatcatcaaatttcgcatatgtttcacgagttgctcacaacatcggcaaatgcgttgttgttccctgtttttacaccttgc
cagggcctggtcaaagcttgacagtttgaccaaattcaggtggcctcatctattcgcactgatagacattgcagatttggaaga
cccagccagtacattacatgcacagccatttgctcctgcaccatgaacttgccacttttgtgcgccggtcgggggtgatagctcg
gcagccgccgatcccaaaggtcccgcggcccaggggcacgagaccccccgacacgattaaatagccaaaatcagtcagaa
cggcacctccaccctacccgaatctgacaaggtcatcaaacgcgcgaaacaacggcgagggtgcgttcgggaagcgcgcgt
agttgacgcaagaagcctgggtcaggctggagggccgcgagaagatcgcttcctgccgagtctgcacccacgcctcgagcgc
accgtccgcgaacaaccaaccccttttcgcgagccctggcattctttcaattgccaaggatgcacatgtgacacgtatagccatt
cggctttgtttgtgcctgcttgactcgcgccatttaattgttttgtgccggtgagccgggagtcggccactcgtctccgagccgca
gtcccggcgccagtcccccggcctctgatctgggtccggaagggttggtataggagcagtctcggctatctgaagcccgttacc
agacactttggccggctgctttccaggcagccgtgtactcttgcgcagtcggtacc.
Nucleotide sequence of PmACCase 3′ flank contained in plasmids pSZ6118,
pSZ6119 and pSZ6120:
(SEQ ID NO: 152)

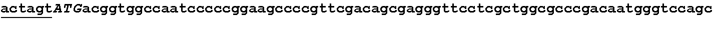

aagcccaccaagctgagctccacccggtccctgctgtccatctcctaccgggagctctcgcgttccaagtgcgtacaggggcg
agggcaccttttgttggtgttgtttgggcgggcctcggtactgggaggaggaggaatgcgtgcacacctctgcggttttagatgc
aatgcgacaagtgcctgctgatgcattttctagacatgaagcatctcgtattcgagtctcaacgcgggtgtgcgctcctcactcc
gtgcagccagcagtcgcggtcgttcacttcgcggggggtgccagggaggacggacgtttcggatgagctggagcgccgcatc
ctcgagtggcagggcgatcgcgccatccacaggtcggttgggtgggaaagggggagtaccggggtcaggtcagaagtcgtg
catttacaggcatgcatctgcacatcgtgcgcacgcgcacgtattggccgcttgtctcaagactcttgcactcgtttcctcatgc
accataatcaattccctcccccctcgcaaactcacagcgtgctggtggccaacaacggtctggcggcggtcaagttcatccggt
cgatccggtcgtggtcgtacaagacgtttgggaacgagcgcgcggtgaagctgattgcgatggcgacgcccgagggcatgcg
cgcggacgcggagcacatccgcatggcggaccagtttgtggaggtccccggcggcaagaacgtgcagaactacgccaacgt
gggcctgatcacctcggtggcggtgcgcaccggggtggacgcggtgcctgcagg.

To determine their impact on fatty acid profiles, the constructs described above were transformed independently into S8414 and S8242. Primary transformants were clonally purified and grown under standard lipid production conditions at pH 7.0. pH 7 was chosen to allow for maximal expression of PmACCase1-1 or PmACCase1-2 genes being upregulated by our pH regulated AMT03 (Ammonium transporter 03) promoter. The resulting profiles from a set of representative clones arising from transformations with pSZ6114 (D5062), pSZ6115 (D5063), pSZ6116 (D5064), pSZ6117 (D5065), pSZ6118 (D5066), pSZ6119 (D5067) and pSZ6120 (D5068) into S8414 and S8242 tables 111-117. In all the transgenic lines either expressing a combination of CrhECR and CrhKCR or CrhHACD and CrkKCR with upregulated PmACCase 1-1 or PmACCase1-2, in both S8414 and S8242 backgrounds, there was a significant increase in C22:1 levels. In S8414 background, the lines S8414; T1435; D5062-6 (18.92%), S8414; T1435; D5063-5 (18.36%), S8414, T1439, D5065-4 (19.15%), the increase in C22:1 levels is 4.03, 3.91 and 4.08 fold over the parent S8414 (4.69%) respectively. The same is true for S8242, T1439; D5063-7 (20.47%) and S8242, T1439; D5065-2 (18.21%) where the increase in C22:1 is 4.06 and 3.62 fold over the parent S8242 (5.03%) respectively. Selected S8414 lines transformed with either D5062, D5063, D5064, D5065, D5066, D5067 or D5068 were run at pH5 and pH7 to regulate the PmAMT03 driven PmACCase1-1 or PmACCase1-2 gene expression (table 118). Decreasing the expression of PmACCase1-1 or PmACCase1-2 by cultivating at pH5.0 led to significant reduction (2.5 or more fold reduction) in C22:1 in all the selected lines confirming the contribution of PmACCase upregulation on very long chain fatty acid biosynthesis (VLCFA) in our host. The reduced C22:1 levels were nevertheless more than the levels in the parent S8414 in almost all the lines thereby demonstrating the positive influence of heterologous KCR and ECR or HACD in VLCFA biosynthesis in P. moriformis (consistent with our results in S7708 background—earlier IP example).

The results disclosed herein demonstrate that increasing the available Malonyl-CoA via upregulation of PmACCase1-1 or PmACCase1-2 along with combined expression of heterologous KCR and ECR or HACD enzyme activities results in significant increase in the VLCFA biosynthesis in P. moriformis strains already expressing a heterologous fatty acid elongase.

TABLE 111
Fatty acid profiles of representative S8414 and
S8242 strains transformed with D5062 (pSZ6114).

| | Fatty acid profile | | | | | |
|---|---|---|---|---|---|---|
| | | | | C18:3 | | |
| Sample ID | C18:0 | C18:1 | C18:2 | □ | C20:1 | C22:1 |
| S8414 | 1.31 | 38.57 | 11.70 | 0.90 | 7.67 | 4.69 |
| S8414; T1435; | 0.75 | 23.73 | 13.11 | 1.37 | 8.91 | 18.92 |
| D5062-6 | | | | | | |
| S8414; T1435; | 1.05 | 28.54 | 12.63 | 1.42 | 8.35 | 13.73 |
| D5062-1 | | | | | | |
| S8414; T1435; | 1.13 | 33.45 | 11.65 | 1.00 | 10.13 | 12.15 |
| D5062-4 | | | | | | |
| S8414; T1435; | 1.10 | 30.86 | 12.41 | 1.32 | 8.50 | 10.63 |
| D5062-7 | | | | | | |
| S8414; T1435; | 1.20 | 40.52 | 11.06 | 0.50 | 9.20 | 6.25 |
| D5062-5 | | | | | | |
| S8242 | 1.77 | 41.06 | 12.69 | 1.17 | 5.85 | 5.03 |
| S8242, T1439; | 1.41 | 32.14 | 12.41 | 1.36 | 7.48 | 14.30 |
| D5062-3 | | | | | | |
| S8242, T1439; | 1.38 | 32.46 | 12.39 | 1.28 | 7.33 | 14.27 |
| D5062-4 | | | | | | |
| S8242, T1439; | 1.43 | 33.50 | 12.02 | 1.11 | 7.58 | 12.79 |
| D5062-1 | | | | | | |
| S8242, T1439; | 1.49 | 33.46 | 12.05 | 1.24 | 7.35 | 12.70 |
| D5062-2 | | | | | | |

TABLE 112
Primary 3-day Fatty acid profiles of representative S8414
and S8242 strains transformed with D5063 (pSZ6115).

| | Fatty acid profile | | | | | |
|---|---|---|---|---|---|---|
| | | | | C18:3 | | |
| Sample ID | C18:0 | C18:1 | C18:2 | □ | C20:1 | C22:1 |
| S8414 | 1.29 | 38.57 | 11.81 | 0.92 | 7.63 | 4.56 |
| S8414; T1435; | 0.95 | 29.36 | 10.91 | 0.72 | 10.88 | 18.36 |
| D5063-5 | | | | | | |
| S8414; T1435; | 0.98 | 28.73 | 12.04 | 1.08 | 9.98 | 13.53 |
| D5063-3 | | | | | | |
| S8414; T1435; | 0.91 | 26.31 | 13.57 | 1.07 | 8.30 | 13.38 |
| D5063-7 | | | | | | |
| S8414; T1435; | 1.04 | 28.94 | 12.73 | 1.35 | 9.23 | 13.18 |
| D5063-9 | | | | | | |
| S8414; T1435; | 1.01 | 32.62 | 11.71 | 1.05 | 8.47 | 10.81 |
| D5063-1 | | | | | | |
| S8242 | 1.75 | 40.66 | 12.63 | 1.16 | 5.79 | 4.81 |
| S8242, T1439; | 1.24 | 27.24 | 11.84 | 1.51 | 8.25 | 20.47 |
| D5063-7 | | | | | | |
| S8242, T1439; | 1.30 | 28.70 | 11.71 | 1.46 | 8.29 | 18.74 |
| D5063-10 | | | | | | |
| S8242, T1439; | 1.28 | 29.14 | 11.81 | 1.45 | 8.29 | 18.30 |
| D5063-3 | | | | | | |
| S8242, T1439; | 1.40 | 29.92 | 11.98 | 1.32 | 8.12 | 17.02 |
| D5063-8 | | | | | | |
| S8242, T1439; | 1.30 | 30.29 | 12.24 | 1.42 | 8.20 | 16.87 |
| D5063-9 | | | | | | |

TABLE 113
Primary 3-day Fatty acid profiles of representative S8414
and S8242 strains transformed with D5064 (pSZ6116).

| | Fatty acid profile | | | | | |
|---|---|---|---|---|---|---|
| | | | | C18:3 | | |
| Sample ID | C18:0 | C18:1 | C18:2 | □ | C20:1 | C22:1 |
| S8414 | 1.29 | 38.57 | 11.81 | 0.92 | 7.63 | 4.56 |
| S8414; T1435; | 1.27 | 31.25 | 12.36 | 1.31 | 10.71 | 14.48 |
| D5064-13 | | | | | | |
| S8414; T1435; | 1.27 | 31.34 | 12.46 | 1.29 | 10.59 | 14.21 |
| D5064-11 | | | | | | |
| S8414; T1435; | 1.32 | 32.45 | 12.43 | 1.28 | 10.55 | 13.36 |
| D5064-15 | | | | | | |
| S8414; T1435; | 1.13 | 29.77 | 11.96 | 1.12 | 8.99 | 12.97 |
| D5064-5 | | | | | | |
| S8414; T1435; | 1.01 | 31.26 | 13.13 | 1.30 | 9.18 | 11.24 |
| D5064-1 | | | | | | |
| S8242 | 1.75 | 40.66 | 12.63 | 1.16 | 5.79 | 4.81 |
| S8242, T1439; | 1.34 | 30.06 | 12.30 | 1.43 | 7.59 | 16.46 |
| D5064-3 | | | | | | |
| S8242, T1439; | 3.44 | 41.31 | 10.11 | 1.03 | 6.15 | 3.51 |
| D5064-1 | | | | | | |
| S8242, T1439; | 2.88 | 43.14 | 10.50 | 1.10 | 4.90 | 1.92 |
| D5064-2 | | | | | | |

TABLE 114
Primary 3-day Fatty acid profiles of representative S8414
and S8242 strains transformed with D5065 (pSZ6117).

| | Fatty acid profile | | | | | |
|---|---|---|---|---|---|---|
| | | | | C18:3 | | |
| Sample ID | C18:0 | C18:1 | C18:2 | □ | C20:1 | C22:1 |
| S8414 | 1.29 | 38.57 | 11.81 | 0.92 | 7.63 | 4.56 |
| S8414; T1435; | 0.79 | 25.39 | 11.77 | 1.02 | 9.70 | 19.15 |
| D5065-4 | | | | | | |
| S8414; T1435; | 0.83 | 27.00 | 12.44 | 1.15 | 10.13 | 16.34 |
| D5065-5 | | | | | | |
| S8414; T1435; | 0.85 | 27.72 | 11.43 | 0.99 | 9.33 | 15.45 |
| D5065-10 | | | | | | |
| S8414; T1435; | 0.94 | 27.09 | 12.72 | 1.24 | 9.33 | 14.68 |
| D5065-8 | | | | | | |
| S8414; T1435; | 0.87 | 27.62 | 13.83 | 1.88 | 8.97 | 14.42 |
| D5065-3 | | | | | | |
| S8242 | 1.75 | 40.66 | 12.63 | 1.16 | 5.79 | 4.81 |
| S8242, T1439; | 1.30 | 29.17 | 12.04 | 1.51 | 8.36 | 18.21 |
| D5065-2 | | | | | | |
| S8242, T1439; | 1.34 | 28.69 | 11.77 | 1.26 | 7.91 | 17.52 |
| D5065-6 | | | | | | |
| S8242, T1439; | 1.40 | 30.48 | 12.01 | 1.38 | 8.25 | 16.95 |
| D5065-4 | | | | | | |
| S8242, T1439; | 1.50 | 32.68 | 11.95 | 1.26 | 7.95 | 13.75 |
| D5065-5 | | | | | | |
| S8242, T1439; | 1.55 | 33.26 | 11.87 | 1.20 | 7.80 | 12.81 |
| D5065-7 | | | | | | |

TABLE 115
Primary 3-day Fatty acid profiles of representative S8414
and S8242 strains transformed with D5066 (pSZ6118).

| | Fatty acid profile | | | | | |
|---|---|---|---|---|---|---|
| | | | | C18:3 | | |
| Sample ID | C18:0 | C18:1 | C18:2 | □ | C20:1 | C22:1 |
| S8414 | 1.29 | 38.57 | 11.81 | 0.92 | 7.63 | 4.56 |
| S8414; T1435; | 0.80 | 22.41 | 15.23 | 1.52 | 9.12 | 17.54 |
| D5066-5 | | | | | | |
| S8414; T1435; | 1.40 | 38.24 | 11.83 | 1.05 | 7.55 | 6.89 |
| D5066-2 | | | | | | |
| S8414; T1435; | 1.27 | 39.55 | 11.88 | 0.83 | 8.60 | 6.55 |
| D5066-11 | | | | | | |
| S8414; T1435; | 1.23 | 38.53 | 12.07 | 0.84 | 9.10 | 6.43 |
| D5066-9 | | | | | | |
| S8414; T1435; | 1.21 | 39.28 | 12.14 | 0.88 | 8.42 | 6.26 |
| D5066-8 | | | | | | |
| S8242 | 1.75 | 40.66 | 12.63 | 1.16 | 5.79 | 4.81 |
| S8242, T1439; | 1.48 | 33.72 | 12.52 | 1.36 | 7.51 | 12.63 |
| D5066-6 | | | | | | |
| S8242, T1439; | 1.46 | 33.55 | 12.83 | 1.34 | 7.55 | 11.89 |
| D5066-3 | | | | | | |
| S8242, T1439; | 1.55 | 34.33 | 12.58 | 1.33 | 7.39 | 11.78 |
| D5066-1 | | | | | | |
| S8242, T1439; | 1.72 | 37.79 | 12.62 | 1.31 | 6.82 | 8.54 |
| D5066-4 | | | | | | |
| S8242, T1439; | 1.63 | 37.39 | 12.70 | 1.29 | 6.96 | 8.28 |
| D5066-7 | | | | | | |

TABLE 116
Primary 3-day Fatty acid profiles of representative S8414
and S8242 strains transformed with D5067 (pSZ6119).

| | Fatty acid profile | | | | | |
|---|---|---|---|---|---|---|
| | | | | C18:3 | | |
| Sample ID | C18:0 | C18:1 | C18:2 | □ | C20:1 | C22:1 |
| S8414 | 1.29 | 38.57 | 11.81 | 0.92 | 7.63 | 4.56 |
| S8414; T1435; | 1.05 | 31.85 | 11.64 | 0.94 | 9.94 | 13.46 |
| D5067-8 | | | | | | |
| S8414; T1435; | 1.05 | 33.66 | 12.72 | 1.13 | 8.81 | 9.01 |
| D5067-1 | | | | | | |
| S8414; T1435; | 1.00 | 32.15 | 13.99 | 1.56 | 9.06 | 8.89 |
| D5067-14 | | | | | | |
| S8414; T1435; | 1.02 | 36.16 | 12.37 | 1.04 | 9.43 | 8.24 |
| D5067-2 | | | | | | |
| S8414; T1435; | 1.06 | 40.21 | 11.99 | 0.82 | 10.41 | 7.86 |
| D5067-3 | | | | | | |
| S8242 | 1.75 | 40.66 | 12.63 | 1.16 | 5.79 | 4.81 |
| S8242, T1439; | 1.26 | 32.50 | 11.80 | 1.28 | 8.13 | 15.84 |
| D5067-1 | | | | | | |

TABLE 117
Primary 3-day Fatty acid profiles of representative S8414
and S8242 strains transformed with D5068 (pSZ6120).

| | Fatty acid profile | | | | | |
|---|---|---|---|---|---|---|
| | | | | C18:3 | | |
| Sample ID | C18:0 | C18:1 | C18:2 | □ | C20:1 | C22:1 |
| S8414 | 1.29 | 38.57 | 11.81 | 0.92 | 7.63 | 4.56 |
| S8414; T1435; | 0.91 | 28.90 | 12.68 | 1.10 | 9.83 | 13.56 |
| D5068-19 | | | | | | |
| S8414; T1435; | 0.89 | 27.90 | 13.13 | 1.39 | 8.99 | 13.56 |
| D5068-3 | | | | | | |
| S8414; T1435; | 1.02 | 35.58 | 15.04 | 0.91 | 11.37 | 12.78 |
| D5068-11 | | | | | | |
| S8414; T1435; | 1.03 | 33.71 | 13.14 | 1.23 | 8.92 | 8.83 |
| D5068-2 | | | | | | |
| S8414; T1435; | 1.11 | 33.86 | 11.93 | 1.07 | 9.11 | 8.65 |
| D5068-18 | | | | | | |
| S8242 | 1.75 | 40.66 | 12.63 | 1.16 | 5.79 | 4.81 |
| S8242, T1439; | 1.27 | 30.29 | 12.73 | 1.52 | 8.18 | 16.18 |
| D5068-6 | | | | | | |
| S8242, T1439; | 1.49 | 31.77 | 13.37 | 1.45 | 7.97 | 12.10 |
| D5068-5 | | | | | | |
| S8242, T1439; | 1.56 | 34.75 | 12.21 | 1.23 | 7.90 | 11.99 |
| D5068-1 | | | | | | |
| S8242, T1439; | 1.86 | 39.96 | 12.64 | 1.27 | 6.77 | 6.61 |
| D5068-2 | | | | | | |
| S8242, T1439; | 1.70 | 39.32 | 13.11 | 1.25 | 6.04 | 5.89 |
| D5068-3 | | | | | | |

TABLE 118
3-day fatty acid profiles of selected S8414 strains
transformed with D5062-D5068 run at pH 5 and pH 7.

| | Fatty acid profile | | | | | |
|---|---|---|---|---|---|---|
| Sample ID | C18:0 | C18:1 | C18:2 | C18:3 a | C20:1 | C22:1 |
| S7485; pH 5 | 3.84 | 50.91 | 5.41 | 0.49 | 0.07 | 0.00 |
| S7485; pH 7 | 4.24 | 45.95 | 5.56 | 0.61 | 0.05 | 0.00 |
| S8414; pH 5 | 1.62 | 47.70 | 9.36 | 0.59 | 6.36 | 2.57 |
| S8414; pH 7 | 1.40 | 38.78 | 11.50 | 0.84 | 7.79 | 4.75 |
| S8414; T1435; | 1.42 | 41.89 | 11.40 | 1.19 | 6.15 | 3.46 |
| D5062-1; | | | | | | |
| pH 5 | | | | | | |
| S8414; T1435; | 1.29 | 32.49 | 11.93 | 1.39 | 8.01 | 10.68 |
| D5062-1; | | | | | | |
| pH 7 | | | | | | |
| S8414; T1435; | 0.95 | 34.40 | 13.89 | 1.66 | 7.78 | 6.57 |
| D5062-6; | | | | | | |
| pH 5 | | | | | | |
| S8414; T1435; | 0.78 | 23.80 | 13.07 | 1.41 | 8.73 | 19.28 |
| D5062-6; | | | | | | |
| pH 7 | | | | | | |
| S8414; T1435; | 1.26 | 44.55 | 10.32 | 0.74 | 7.59 | 3.78 |
| D5063-3; | | | | | | |
| pH 5 | | | | | | |
| S8414; T1435; | 1.08 | 29.92 | 11.69 | 1.07 | 9.98 | 13.25 |
| D5063-3; | | | | | | |
| pH 7 | | | | | | |
| S8414; T1435; | 1.25 | 43.54 | 9.96 | 0.65 | 9.17 | 5.49 |
| D5063-5; | | | | | | |
| pH 5 | | | | | | |
| S8414; T1435; | 1.01 | 30.05 | 10.79 | 0.73 | 10.94 | 18.25 |
| D5063-5; | | | | | | |
| pH 7 | | | | | | |
| S8414; T1435; | 1.86 | 48.14 | 10.94 | 0.91 | 8.31 | 3.93 |
| D5064-11; | | | | | | |
| pH 5 | | | | | | |
| S8414; T1435; | 1.40 | 32.79 | 11.97 | 1.20 | 10.75 | 13.92 |
| D5064-11; | | | | | | |
| pH 7 | | | | | | |
| S8414; T1435; | 1.80 | 47.75 | 11.06 | 0.96 | 8.43 | 4.07 |
| D5064-13; | | | | | | |
| pH 5 | | | | | | |
| S8414; T1435; | 1.36 | 32.26 | 12.13 | 1.21 | 10.88 | 14.26 |
| D5064-13; | | | | | | |
| pH 7 | | | | | | |
| S8414; T1435; | 0.99 | 39.35 | 10.84 | 0.81 | 8.95 | 6.79 |
| D5065-4; | | | | | | |
| pH 5 | | | | | | |
| S8414; T1435; | 0.88 | 26.65 | 11.74 | 1.00 | 9.88 | 17.90 |
| D5065-4; | | | | | | |
| pH 7 | | | | | | |
| S8414; T1435; | 1.14 | 42.90 | 10.80 | 0.79 | 8.08 | 4.58 |
| D5065-5; | | | | | | |
| pH 5 | | | | | | |
| S8414; T1435; | 0.98 | 28.01 | 12.04 | 1.13 | 10.06 | 15.53 |
| D5065-5; | | | | | | |
| pH 7 | | | | | | |
| S8414; T1435; | 1.71 | 47.24 | 9.94 | 0.82 | 5.95 | 2.93 |
| D5066-2; | | | | | | |
| pH 5 | | | | | | |
| S8414; T1435; | 1.74 | 39.55 | 11.02 | 0.95 | 7.04 | 6.61 |
| D5066-2; | | | | | | |
| pH 7 | | | | | | |
| S8414; T1435; | 1.01 | 34.20 | 15.15 | 1.35 | 8.58 | 7.12 |
| D5066-5; | | | | | | |
| pH 5 | | | | | | |
| S8414; T1435; | 0.81 | 22.84 | 15.16 | 1.65 | 9.34 | 18.13 |
| D5066-5; | | | | | | |
| pH 7 | | | | | | |
| S8414; T1435; | 1.27 | 44.50 | 10.40 | 0.73 | 7.52 | 4.00 |
| D5067-8; | | | | | | |
| pH 5 | | | | | | |
| S8414; T1435; | 1.11 | 30.78 | 11.82 | 1.04 | 9.66 | 12.96 |
| D5067-8; | | | | | | |
| pH 7 | | | | | | |
| S8414; T1435; | 1.18 | 39.69 | 10.23 | 1.05 | 9.48 | 6.67 |
| D5067-14; | | | | | | |
| pH 5 | | | | | | |
| S8414; T1435; | 1.08 | 32.21 | 13.71 | 1.57 | 9.38 | 9.40 |
| D5067-14; | | | | | | |
| pH 7 | | | | | | |
| S8414; T1435; | 1.37 | 51.76 | 13.81 | 0.81 | 6.90 | 2.65 |
| D5068-11; | | | | | | |
| pH 5 | | | | | | |
| S8414; T1435; | 1.07 | 35.67 | 15.27 | 0.88 | 11.13 | 12.50 |
| D5068-11; | | | | | | |
| pH 7 | | | | | | |
| S8414; T1435; | 1.15 | 42.32 | 10.69 | 0.79 | 8.36 | 5.01 |
| D5068-19; | | | | | | |
| pH 5 | | | | | | |
| S8414; T1435; | 1.03 | 30.35 | 12.71 | 1.10 | 9.79 | 12.52 |
| D5068-19; | | | | | | |
| pH 7 | | | | | | |

SEQUENCES
6S 5′ genomic donor sequence
SEQ ID NO: 1
GCTCTTCGCCGCCGCCACTCCTGCTCGAGCGCGCCCGCGCGTGCGCCGCCAGCGCCTTGGCCTTTTCG
CCGCGCTCGTGCGCGTCGCTGATGTCCATCACCAGGTCCATGAGGTCTGCCTTGCGCCGGCTGAGCCA
CTGCTTCGTCCGGGCGGCCAAGAGGAGCATGAGGGAGGACTCCTGGTCCAGGGTCCTGACGTGGTCGC
GGCTCTGGGAGCGGGCCAGCATCATCTGGCTCTGCCGCACCGAGGCCGCCTCCAACTGGTCCTCCAGC
AGCCGCAGTCGCCGCCGACCCTGGCAGAGGAAGACAGGTGAGGGGGGTATGAATTGTACAGAACAACC
ACGAGCCTTGTCTAGGCAGAATCCCTACCAGTCATGGCTTTACCTGGATGACGGCCTGCGAACAGCTG
TCCAGCGACCCTCGCTGCCGCCGCTTCTCCCGCACGCTTCTTTCCAGCACCGTGATGGCGCGAGCCAG
CGCCGCACGCTGGCGCTGCGCTTCGCCGATCTGAGGACAGTCGGGGAACTCTGATCAGTCTAAACCCC
CTTGCGCGTTAGTGTTGCCATCCTTTGCAGACCGGTGAGAGCCGACTTGTTGTGCGCCACCCCCCACA
CCACCTCCTCCCAGACCAATTCTGTCACCTTTTTGGCGAAGGCATCGGCCTCGGCCTGCAGAGAGGAC
AGCAGTGCCCAGCCGCTGGGGGTTGGCGGATGCACGCTCAGGTACC
6S 3′ genomic donor sequence
SEQ ID NO: 2
GAGCTCCTTGTTTTCCAGAAGGAGTTGCTCCTTGAGCCTTTCATTCTCAGCCTCGATAACCTCCAAAG
CCGCTCTAATTGTGGAGGGGGTTCGAATTTAAAAGCTTGGAATGTTGGTTCGTGCGTCTGGAACAAGC
CCAGACTTGTTGCTCACTGGGAAAAGGACCATCAGCTCCAAAAAACTTGCCGCTCAAACCGCGTACCT
CTGCTTTCGCGCAATCTGCCCTGTTGAAATCGCCACCACATTCATATTGTGACGCTTGAGCAGTCTGT
AATTGCCTCAGAATGTGGAATCATCTGCCCCCTGTGCGAGCCCATGCCAGGCATGTCGCGGGCGAGGA
CACCCGCCACTCGTACAGCAGACCATTATGCTACCTCACAATAGTTCATAACAGTGACCATATTTCTC
GAAGCTCCCCAACGAGCACCTCCATGCTCTGAGTGGCCACCCCCCGGCCCTGGTGCTTGCGGAGGGCA
GGTCAACCGGCATGGGGCTACCGAAATCCCCGACCGGATCCCACCACCCCCGCGATGGGAAGAATCTC
TCCCCGGGATGTGGGCCCACCACCAGCACAACCTGCTGGCCCAGGCGAGCGTCAAACCATACCACACA
AATATCCTTGGCATCGGCCCTGAATTCCTTCTGCCGCTCTGCTACCCGGTGCTTCTGTCCGAAGCAGG
GGTTGCTAGGGATCGCTCCGAGTCCGCAAACCCTTGTCGCGTGGCGGGGCTTGTTCGAGCTTGAAGAG
C
S. cereviseae invertase protein sequence
SEQ ID NO: 3
MLLQAFLFLLAGFAAKISASMTNETSDRPLVHFTPNKGWMNDPNGLWYDEKDAKWHLYFQYNPNDTVW
GTPLFWGHATSDDLTNWEDQPIAIAPKRNDSGAFSGSMVVDYNNTSGFFNDTIDPRQRCVAIWTYNTP
ESEEQYISYSLDGGYTFTEYQKNPVLAANSTQFRDPKVFWYEPSQKWIMTAAKSQDYKIEIYSSDDLK
SWKLESAFANEGFLGYQYECPGLIEVPTEQDPSKSYWVMFISINPGAPAGGSFNQYFVGSFNGTHFEA
FDNQSRVVDFGKDYYALQTFFNTDPTYGSALGIAWASNWEYSAFVPTNPWRSSMSLVRKFSLNTEYQA
NPETELINLKAEPILNISNAGPWSRFATNTTLTKANSYNVDLSNSTGTLEFELVYAVNTTQTISKSVF
ADLSLWFKGLEDPEEYLRMGFEVSASSFFLDRGNSKVKFVKENPYFTNRMSVNNQPFKSENDLSYYKV
YGLLDQNILELYFNDGDVVSTNTYFMTTGNALGSVNMTTGVDNLFYIDKFQVREVK
S. cereviseae invertase protein coding sequence codon optimized for
expression in P. moriformis (UTEX 1435)
SEQ ID NO: 4
ATGctgctgcaggccttcctgttcctgctggccggcttcgccgccaagatcagcgcctccatgacgaa
cgagacgtccgaccgccccctggtgcacttcacccccaacaagggctggatgaacgaccccaacggcc
tgtggtacgacgagaaggacgccaagtggcacctgtacttccagtacaacccgaacgacaccgtctgg
gggacgcccttgttctggggccacgccacgtccgacgacctgaccaactgggaggaccagcccatcgc
catcgccccgaagcgcaacgactccggcgccttctccggctccatggtggtggactacaacaacacct
ccggcttcttcaacgacaccatcgacccgcgccagcgctgcgtggccatctggacctacaacaccccg
gagtccgaggagcagtacatctcctacagcctggacggcggctacaccttcaccgagtaccagaagaa
ccccgtgctggccgccaactccacccagttccgcgacccgaaggtcttctggtacgagccctcccaga
agtggatcatgaccgcggccaagtcccaggactacaagatcgagatctactcctccgacgacctgaag
tcctggaagctggagtccgcgttcgccaacgagggcttcctcggctaccagtacgagtgccccggcct
gatcgaggtccccaccgagcaggaccccagcaagtcctactgggtgatgttcatctccatcaaccccg
gcgccccggccggcggctccttcaaccagtacttcgtcggcagcttcaacggcacccacttcgaggcc
ttcgacaaccagtcccgcgtggtggacttcggcaaggactactacgccctgcagaccttcttcaacac
cgacccgacctacgggagcgccctgggcatcgcgtgggcctccaactgggagtactccgccttcgtgc
ccaccaacccctggcgctcctccatgtccctcgtgcgcaagttctccctcaacaccgagtaccaggcc
aacccggagacggagctgatcaacctgaaggccgagccgatcctgaacatcagcaacgccggcccctg
gagccggttcgccaccaacaccacgttgacgaaggccaacagctacaacgtcgacctgtccaacagca
ccggcaccctggagttcgagctggtgtacgccgtcaacaccacccagacgatctccaagtccgtgttc
gcggacctctccctctggttcaagggcctggaggaccccgaggagtacctccgcatgggcttcgaggt
gtccgcgtcctccttcttcctggaccgcgggaacagcaaggtgaagttcgtgaaggagaacccctact
tcaccaaccgcatgagcgtgaacaaccagcccttcaagagcgagaacgacctgtcctactacaaggtg
tacggcttgctggaccagaacatcctggagctgtacttcaacgacggcgacgtcgtgtccaccaacac
ctacttcatgaccaccgggaacgccctgggctccgtgaacatgacgacgggggtggacaacctgttct
acatcgacaagttccaggtgcgcgaggtcaagTGA
Chlamydomonas reinhardtii TUB2 (B-tub) promoter/5′ UTR
SEQ ID NO: 5
CTTTCTTGCGCTATGACACTTCCAGCAAAAGGTAGGGCGGGCTGCGAGACGGCTTCCCGGCGCTGCAT
GCAACACCGATGATGCTTCGACCCCCCGAAGCTCCTTCGGGGCTGCATGGGCGCTCCGATGCCGCTCC
AGGGCGAGCGCTGTTTAAATAGCCAGGCCCCCGATTGCAAAGACATTATAGCGAGCTACCAAAGCCAT
ATTCAAACACCTAGATCACTACCACTTCTACACAGGCCACTCGAGCTTGTGATCGCACTCCGCTAAGG
GGGCGCCTCTTCCTCTTCGTTTCAGTCACAACCCGCAAAC
Chlorella vulgaris nitrate reductase 3′ UTR
SEQ ID NO: 6
GCAGCAGCAGCTCGGATAGTATCGACACACTCTGGACGCTGGTCGTGTGATGGACTGTTGCCGCCACA
CTTGCTGCCTTGACCTGTGAATATCCCTGCCGCTTTTATCAAACAGCCTCAGTGTGTTTGATCTTGTG
TGTACGCGCTTTTGCGAGTTGCTAGCTGCTTGTGCTATTTGCGAATACCACCCCCAGCATCCCCTTCC
CTCGTTTCATATCGCTTGCATCCCAACCGCAACTTATCTACGCTGTCCTGCTATCCCTCAGCGCTGCT
CCTGCTCCTGCTCACTGCCCCTCGCACAGCCTTGGTTTGGGCTCCGCCTGTATTCTCCTGGTACTGCA
ACCTGTAAACCAGCACTGCAATGCTGATGCACGGGAAGTAGTGGGATGGGAACACAAATGGAAAGCTT
Nucleotidc sequence of thc codon-optimized expression cassette of S.
cerevisiae suc2 gene with C. reinhardtii β-tubulin promoter/5′ UTR
and C. vulgaris nitrate reductase 3′ UTR
SEQ ID NO: 7
CTTTCTTGCGCTATGACACTTCCAGCAAAAGGTAGGGCGGGCTGCGAGACGGCTTCCCGGCGCTGCAT
GCAACACCGATGATGCTTCGACCCCCCGAAGCTCCTTCGGGGCTGCATGGGCGCTCCGATGCCGCTCC
AGGGCGAGCGCTGTTTAAATAGCCAGGCCCCCGATTGCAAAGACATTATAGCGAGCTACCAAAGCCAT
ATTCAAACACCTAGATCACTACCACTTCTACACAGGCCACTCGAGCTTGTGATCGCACTCCGCTAAGG
GGGCGCCTCTTCCTCTTCGTTTCAGTCACAACCCGCAAACGGCGCGCCATGCTGCTGCAGGCCTTCCT
GTTCCTGCTGGCCGGCTTCGCCGCCAAGATCAGCGCCTCCATGACGAACGAGACGTCCGACCGCCCCC
TGGTGCACTTCACCCCCAACAAGGGCTGGATGAACGACCCCAACGGCCTGTGGTACGACGAGAAGGAC
GCCAAGTGGCACCTGTACTTCCAGTACAACCCGAACGACACCGTCTGGGGGACGCCCTTGTTCTGGGG
CCACGCCACGTCCGACGACCTGACCAACTGGGAGGACCAGCCCATCGCCATCGCCCCGAAGCGCAACG
ACTCCGGCGCCTTCTCCGGCTCCATGGTGGTGGACTACAACAACACCTCCGGCTTCTTCAACGACACC
ATCGACCCGCGCCAGCGCTGCGTGGCCATCTGGACCTACAACACCCCGGAGTCCGAGGAGCAGTACAT
CTCCTACAGCCTGGACGGCGGCTACACCTTCACCGAGTACCAGAAGAACCCCGTGCTGGCCGCCAACT
CCACCCAGTTCCGCGACCCGAAGGTCTTCTGGTACGAGCCCTCCCAGAAGTGGATCATGACCGCGGCC
AAGTCCCAGGACTACAAGATCGAGATCTACTCCTCCGACGACCTGAAGTCCTGGAAGCTGGAGTCCGC
GTTCGCCAACGAGGGCTTCCTCGGCTACCAGTACGAGTGCCCCGGCCTGATCGAGGTCCCCACCGAGC
AGGACCCCAGCAAGTCCTACTGGGTGATGTTCATCTCCATCAACCCCGGCGCCCCGGCCGGCGGCTCC
TTCAACCAGTACTTCGTCGGCAGCTTCAACGGCACCCACTTCGAGGCCTTCGACAACCAGTCCCGCGT
GGTGGACTTCGGCAAGGACTACTACGCCCTGCAGACCTTCTTCAACACCGACCCGACCTACGGGAGCG
CCCTGGGCATCGCGTGGGCCTCCAACTGGGAGTACTCCGCCTTCGTGCCCACCAACCCCTGGCGCTCC
TCCATGTCCCTCGTGCGCAAGTTCTCCCTCAACACCGAGTACCAGGCCAACCCGGAGACGGAGCTGAT
CAACCTGAAGGCCGAGCCGATCCTGAACATCAGCAACGCCGGCCCCTGGAGCCGGTTCGCCACCAACA
CCACGTTGACGAAGGCCAACAGCTACAACGTCGACCTGTCCAACAGCACCGGCACCCTGGAGTTCGAG
CTGGTGTACGCCGTCAACACCACCCAGACGATCTCCAAGTCCGTGTTCGCGGACCTCTCCCTCTGGTT
CAAGGGCCTGGAGGACCCCGAGGAGTACCTCCGCATGGGCTTCGAGGTGTCCGCGTCCTCCTTCTTCC
TGGACCGCGGGAACAGCAAGGTGAAGTTCGTGAAGGAGAACCCCTACTTCACCAACCGCATGAGCGTG
AACAACCAGCCCTTCAAGAGCGAGAACGACCTGTCCTACTACAAGGTGTACGGCTTGCTGGACCAGAA
CATCCTGGAGCTGTACTTCAACGACGGCGACGTCGTGTCCACCAACACCTACTTCATGACCACCGGGA
ACGCCCTGGGCTCCGTGAACATGACGACGGGGGTGGACAACCTGTTCTACATCGACAAGTTCCAGGTG
CGCGAGGTCAAGTGACAATTGGCAGCAGCAGCTCGGATAGTATCGACACACTCTGGACGCTGGTCGTG
TGATGGACTGTTGCCGCCACACTTGCTGCCTTGACCTGTGAATATCCCTGCCGCTTTTATCAAACAGC
CTCAGTGTGTTTGATCTTGTGTGTACGCGCTTTTGCGAGTTGCTAGCTGCTTGTGCTATTTGCGAATA
CCACCCCCAGCATCCCCTTCCCTCGTTTCATATCGCTTGCATCCCAACCGCAACTTATCTACGCTGTC
CTGCTATCCCTCAGCGCTGCTCCTGCTCCTGCTCACTGCCCCTCGCACAGCCTTGGTTTGGGCTCCGC
CTGTATTCTCCTGGTACTGCAACCTGTAAACCAGCACTGCAATGCTGATGCACGGGAAGTAGTGGGAT
GGGAACACAAATGGAGGATCC
Prototheca moriformis (UTEX 1435) Amt03 promoter
SEQ ID NO: 8
GGCCGACAGGACGCGCGTCAAAGGTGCTGGTCGTGTATGCCCTGGCCGGCAGGTCGTTGCTGCTGCTG
GTTAGTGATTCCGCAACCCTGATTTTGGCGTCTTATTTTGGCGTGGCAAACGCTGGCGCCCGCGAGCC
GGGCCGGCGGCGATGCGGTGCCCCACGGCTGCCGGAATCCAAGGGAGGCAAGAGCGCCCGGGTCAGTT
GAAGGGCTTTACGCGCAAGGTACAGCCGCTCCTGCAAGGCTGCGTGGTGGAATTGGACGTGCAGGTCC
TGCTGAAGTTCCTCCACCGCCTCACCAGCGGACAAAGCACCGGTGTATCAGGTCCGTGTCATCCACTC
TAAAGAGCTCGACTACGACCTACTGATGGCCCTAGATTCTTCATCAAAAACGCCTGAGACACTTGCCC
AGGATTGAAACTCCCTGAAGGGACCACCAGGGGCCCTGAGTTGTTCCTTCCCCCCGTGGCGAGCTGCC
AGCCAGGCTGTACCTGTGATCGAGGCTGGCGGGAAAATAGGCTTCGTGTGCTCAGGTCATGGGAGGTG
CAGGACAGCTCATGAAACGCCAACAATCGCACAATTCATGTCAAGCTAATCAGCTATTTCCTCTTCAC
GAGCTGTAATTGTCCCAAAATTCTGGTCTACCGGGGGTGATCCTTCGTGTACGGGCCCTTCCCTCAAC
CCTAGGTATGCGCGCATGCGGTCGCCGCGCAACTCGCGCGAGGGCCGAGGGTTTGGGACGGGCCGTCC
CGAAATGCAGTTGCACCCGGATGCGTGGCACCTTTTTTGCGATAATTTATGCAATGGACTGCTCTGCA
AAATTCTGGCTCTGTCGCCAACCCTAGGATCAGCGGCGTAGGATTTCGTAATCATTCGTCCTGATGGG
GAGCTACCGACTACCCTAATATCAGCCCGACTGCCTGACGCCAGCGTCCACTTTTGTGCACACATTCC
ATTCGTGCCCAAGACATTTCATTGTGGTGCGAAGCGTCCCCAGTTACGCTCACCTGTTTCCCGACCTC
CTTACTGTTCTGTCGACAGAGCGGGCCCACAGGCCGGTCGCAGCC
Chlorella protothecoides (UTEX 250) stearoyl ACP desaturase transit
peptide cDNA sequence codon optimized for expression in P.
moriformis.
SEQ ID NO: 9
ACTAGTATGGCCACCGCATCCACTTTCTCGGCGTTCAATGCCCGCTGCGGCGACCTGCGTCGCTCGGC
GGGCTCCGGGCCCCGGCGCCCAGCGAGGCCCCTCCCCGTGCGCGGGCGCGCC
Cuphea wrightii FatB2 thioesterase nucleic acid sequence; Gen Bank
Accession No. U56104
SEQ ID NO: 10
ATGGTGGTGGCCGCCGCCGCCAGCAGCGCCTTCTTCCCCGTGCCCGCCCCCCGCCCCACCCCCAAGCC
CGGCAAGTTCGGCAACTGGCCCAGCAGCCTGAGCCAGCCCTTCAAGCCCAAGAGCAACCCCAACGGCC
GCTTCCAGGTGAAGGCCAACGTGAGCCCCCACGGGCGCGCCCCCAAGGCCAACGGCAGCGCCGTGAGC
CTGAAGTCCGGCAGCCTGAACACCCTGGAGGACCCCCCCAGCAGCCCCCCCCCCCGCACCTTCCTGAA
CCAGCTGCCCGACTGGAGCCGCCTGCGCACCGCCATCACCACCGTGTTCGTGGCCGCCGAGAAGCAGT
TCACCCGCCTGGACCGCAAGAGCAAGCGCCCCGACATGCTGGTGGACTGGTTCGGCAGCGAGACCATC
GTGCAGGACGGCCTGGTGTTCCGCGAGCGCTTCAGCATCCGCAGCTACGAGATCGGCGCCGACCGCAC
CGCCAGCATCGAGACCCTGATGAACCACCTGCAGGACACCAGCCTGAACCACTGCAAGAGCGTGGGCC
TGCTGAACGACGGCTTCGGCCGCACCCCCGAGATGTGCACCCGCGACCTGATCTGGGTGCTGACCAAG
ATGCAGATCGTGGTGAACCGCTACCCCACCTGGGGCGACACCGTGGAGATCAACAGCTGGTTCAGCCA
GAGCGGCAAGATCGGCATGGGCCGCGAGTGGCTGATCAGCGACTGCAACACCGGCGAGATCCTGGTGC
GCGCCACCAGCGCCTGGGCCATGATGAACCAGAAGACCCGCCGCTTCAGCAAGCTGCCCTGCGAGGTG
CGCCAGGAGATCGCCCCCCACTTCGTGGACGCCCCCCCCGTGATCGAGGACAACGACCGCAAGCTGCA
CAAGTTCGACGTGAAGACCGGCGACAGCATCTGCAAGGGCCTGACCCCCGGCTGGAACGACTTCGACG
TGAACCAGCACGTGAGCAACGTGAAGTACATCGGCTGGATTCTGGAGAGCATGCCCACCGAGGTGCTG
GAGACCCAGGAGCTGTGCAGCCTGACCCTGGAGTACCGCCGCGAGTGCGGCCGCGAGAGCGTGGTGGA
GAGCGTGACCAGCATGAACCCCAGCAAGGTGGGCGACCGCAGCCAGTACCAGCACCTGCTGCGCCTGG
AGGACGGCGCCGACATCATGAAGGGCCGCACCGAGTGGCGCCCCAAGAACGCCGGCACCAACCGCGCC
ATCAGCACCTGA
Cuphea wrightii FatB2 thioesterase amino acid sequence; Gen Bank
Accession No. U56104
SEQ ID NO: 11
MVVAAAASSAFFPVPAPRPTPKPGKFGNWPSSLSQPFKPKSNPNGRFQVKANVSPHPKANGSAVSLKS
GSLNTLEDPPSSPPPRTFLNQLPDWSRLRTAITTVFVAAEKQFTRLDRKSKRPDMLVDWFGSETIVQD
GLVFRERFSIRSYEIGADRTASIETLMNHLQDTSLNHCKSVGLLNDGFGRTPEMCTRDLIWVLTKMQI
VVNRYPTWGDTVEINSWFSQSGKIGMGREWLISDCNTGEILVRATSAWAMMNQKTRRFSKLPCEVRQE
IAPHFVDAPPVIEDNDRKLHKFDVKTGDSICKGLTPGWNDFDVNQHVSNVKYIGWILESMPTEVLETQ
ELCSLTLEYRRECGRESVVESVTSMNPSKVGDRSQYQHLLRLEDGADIMKGRTEWRPKNAGTNRAIST
Codon-optimized coding region of Cocus nucifera C12:0-preferring
LPAAT from pSZ2046
SEQ ID NO: 12
ATGGACGCCTCCGGCGCCTCCTCCTTCCTGCGCGGCCGCTGCCTGGAGTCCTGCTTCAAGGCCTCCTT
CGGCTACGTAATGTCCCAGCCCAAGGACGCCGCCGGCCAGCCCTCCCGCCGCCCCGCCGACGCCGACG
ACTTCGTGGACGACGACCGCTGGATCACCGTGATCCTGTCCGTGGTGCGCATCGCCGCCTGCTTCCTG
TCCATGATGGTGACCACCATCGTGTGGAACATGATCATGCTGATCCTGCTGCCCTGGCCCTACGCCCG
CATCCGCCAGGGCAACCTGTACGGCCACGTGACCGGCCGCATGCTGATGTGGATTCTGGGCAACCCCA
TCACCATCGAGGGCTCCGAGTTCTCCAACACCCGCGCCATCTACATCTGCAACCACGCCTCCCTGGTG
GACATCTTCCTGATCATGTGGCTGATCCCCAAGGGCACCGTGACCATCGCCAAGAAGGAGATCATCTG
GTATCCCCTGTTCGGCCAGCTGTACGTGCTGGCCAACCACCAGCGCATCGACCGCTCCAACCCCTCCG
CCGCCATCGAGTCCATCAAGGAGGTGGCCCGCGCCGTGGTGAAGAAGAACCTGTCCCTGATCATCTTC
CCCGAGGGCACCCGCTCCAAGACCGGCCGCCTGCTGCCCTTCAAGAAGGGCTTCATCCACATCGCCCT
CCAGACCCGCCTGCCCATCGTGCCGATGGTGCTGACCGGCACCCACCTGGCCTGGCGCAAGAACTCCC
TGCGCGTGCGCCCCGCCCCCATCACCGTGAAGTACTTCTCCCCCATCAAGACCGACGACTGGGAGGAG
GAGAAGATCAACCACTACGTGGAGATGATCCACGCCCTGTACGTGGACCACCTGCCCGAGTCCCAGAA
GCCCCTGGTGTCCAAGGGCCGCGACGCCTCCGGCCGCTCCAACTCCTGA
pLoop 5′ genomic donor sequence
SEQ ID NO: 13
gctcttcgctaacggaggtctgtcaccaaatggaccccgtctattgcgggaaaccacggcgatggcac
gtttcaaaacttgatgaaatacaatattcagtatgtcgcgggcggcgacggcggggagctgatgtcgc
gctgggtattgcttaatcgccagcttcgcccccgtcttggcgcgaggcgtgaacaagccgaccgatgt
gcacgagcaaatcctgacactagaagggctgactcgcccggcacggctgaattacacaggcttgcaaa
aataccagaatttgcacgcaccgtattcgcggtattttgttggacagtgaatagcgatgcggcaatgg
cttgtggcgttagaaggtgcgacgaaggtggtgccaccactgtgccagccagtcctggcggctcccag
ggccccgatcaagagccaggacatccaaactacccacagcatcaacgccccggcctatactcgaaccc
cacttgcactctgcaatggtatgggaaccacggggcagtcttgtgtgggtcgcgcctatcgcggtcgg
cgaagaccgggaaggtacc
pLoop 3′ genomic donor sequence
SEQ ID NO: 14
gagctcagcggcgacggtcctgctaccgtacgacgttgggcacgcccatgaaagtttgtataccgagc
ttgttgagcgaactgcaagcgcggctcaaggatacttgaactcctggattgatatcggtccaataatg
gatggaaaatccgaacctcgtgcaagaactgagcaaacctcgttacatggatgcacagtcgccagtcc
aatgaacattgaagtgagcgaactgttcgcttcggtggcagtactactcaaagaatgagctgctgtta
aaaatgcactctcgttctctcaagtgagtggcagatgagtgctcacgccttgcacttcgctgcccgtg
tcatgccctgcgccccaaaatttgaaaaaagggatgagattattgggcaatggacgacgtcgtcgctc
cgggagtcaggaccggcggaaaataagaggcaacacactccgcttcttagctcttcc
NeoR expression cassette including C. reinhardtii β-tubulin
promoter/5′UTR and C. vulgaris nitrate reductase 3′ UTR
SEQ ID NO: 15

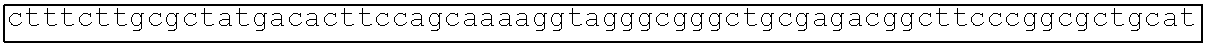

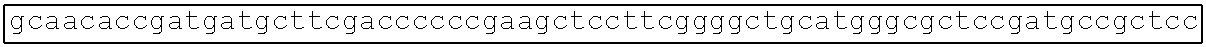

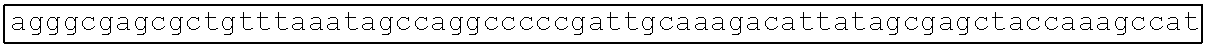

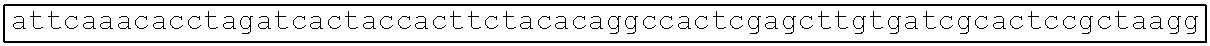

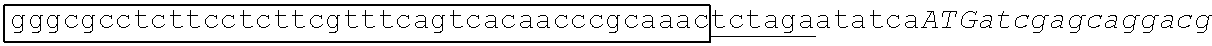

gcctccacgccggctcccccgccgcctgggtggagcgcctgttcggctacgactgggcccagcagacc
atcggctgctccgacgccgccgtgttccgcctgtccgcccagggccgccccgtgctgttcgtgaagac
cgacctgtccggcgccctgaacgagctgcaggacgaggccgcccgcctgtcctggctggccaccaccg
gcgtgccctgcgccgccgtgctggacgtggtgaccgaggccggccgcgactggctgctgctgggcgag
gtgcccggccaggacctgctgtcctcccacctggcccccgccgagaaggtgtccatcatggccgacgc
catgcgccgcctgcacaccctggaccccgccacctgccccttcgaccaccaggccaagcaccgcatcg
agcgcgcccgcacccgcatggaggccggcctggtggaccaggacgacctggacgaggagcaccagggc
ctggcccccgccgagctgttcgcccgcctgaaggcccgcatgcccgacggcgaggacctggtggtgac
ccacggcgacgcctgcctgcccaacatcatggtggagaacggccgcttctccggcttcatcgactgcg
gccgcctgggcgtggccgaccgctaccaggacatcgccctggccacccgcgacatcgccgaggagctg
ggcggcgagtgggccgaccgcttcctggtgctgtacggcatcgccgcccccgactcccagcgcatcgc
cttctaccgcctgctggacgagttcttcTGAcaattggcagcagcagctcggatagtatcgacacact
ctggacgctggtcgtgtgatggactgttgccgccacacttgctgccttgacctgtgaatatccctgcc
gcttttatcaaacagcctcagtgtgtttgatcttgtgtgtacgcgcttttgcgagttgctagctgctt
gtgctatttgcgaataccacccccagcatccccttccctcgtttcatatcgcttgcatcccaaccgca
acttatctacgctgtcctgctatccctcagcgctgctcctgctcctgctcactgcccctcgcacagcc
ttggtttgggctccgcctgtattctcctggtactgcaacctgtaaaccagcactgcaatgctgatgca
cgggaagtagtgggatgggaacacaaatggaggatcc
Cocos nucifera 1-acyl-sn-glycerol-3-phosphatc acyltransferase
(LPAAT)
SEQ ID NO: 16
MDASGASSFLRGRCLESCFKASFGYVMSQPKDAAGQPSRRPADADDFVDDDRWITVILSV
VRIAACFLSMMVITIVWNMIMLILLPWPYARIRQGNLYGHVTGRMLMWILGNPITIEGSE
FSNTRAIYICNHASLVDIFLIMWLIPKGIVTIAKKEIIWYPLFGQLYVLANHQRIDRSNP
SAAIESIKEVARAVVKKNLSLIIFPEGIRSKTGRLLPFKKGFIHIALQTRLPIVPMVLIG
THLAWRKNSLRVRPAPITVKYFSPIKTDDWEEEKINHYVEMIHALYVDHLPESQKPLVSK
GRDASGRSNS
PmKASII (Prototheca moriformis KASII) comprising a C. protothecoides
S106 stearoyl-ACP desaturase transit peptide
SEQ ID NO: 17
ATGgccaccgcatccactttctcggcgttcaatgcccgctgcggcgacctgcgtcgctcggcgggctc
cgggccccggcgcccagcgaggcccctccccgtgcgcgggcgcgccgccgccgccgccgacgccaacc
ccgcccgccccgagcgccgcgtggtgatcaccggccagggcgtggtgacctccctgggccagaccatc
gagcagttctactcctccctgctggagggcgtgtccggcatctcccagatccagaagttcgacaccac
cggctacaccaccaccatcgccggcgagatcaagtccctgcagctggacccctacgtgcccaagcgct
gggccaagcgcgtggacgacgtgatcaagtacgtgtacatcgccggcaagcaggccctggagtccgcc
ggcctgcccatcgaggccgccggcctggccggcgccggcctggaccccgccctgtgcggcgtgctgat
cggcaccgccatggccggcatgacctccttcgccgccggcgtggaggccctgacccgcggcggcgtgc
gcaagatgaaccccttctgcatccccttctccatctccaacatgggcggcgccatgctggccatggac
atcggcttcatgggccccaactactccatctccaccgcctgcgccaccggcaactactgcatcctggg
cgccgccgaccacatccgccgcggcgacgccaacgtgatgctggccggcggcgccgacgccgccatca
tcccctccggcatcggcggcttcatcgcctgcaaggccctgtccaagcgcaacgacgagcccgagcgc
gcctcccgcccctgggacgccgaccgcgacggcttcgtgatgggcgagggcgccggcgtgctggtgct
ggaggagctggagcacgccaagcgccgcggcgccaccatcctggccgagctggtgggcggcgccgcca
cctccgacgcccaccacatgaccgagcccgacccccagggccgcggcgtgcgcctgtgcctggagcgc
gccctggagcgcgcccgcctggcccccgagcgcgtgggctacgtgaacgcccacggcacctccacccc
cgccggcgacgtggccgagtaccgcgccatccgcgccgtgatcccccaggactccctgcgcatcaact
ccaccaagtccatgatcggccacctgctgggcggcgccggcgccgtggaggccgtggccgccatccag
gccctgcgcaccggctggctgcaccccaacctgaacctggagaaccccgcccccggcgtggaccccgt
ggtgctggtgggcccccgcaaggagcgcgccgaggacctggacgtggtgctgtccaactccttcggct
tcggcggccacaactcctgcgtgatcttccgcaagtacgacgagatggactacaaggaccacgacggc
gactacaaggaccacgacatcgactacaaggacgacgacgacaagTGA
PmKASII (Prototheca moriformis KASII) comprising a C. protothecoides
S106 stearoylACP desaturase transit peptide
SEQ ID NO: 18
MATASTFSAFNARCGDLRRSAGSGPRRPARPLPVRGRAAAAADANPARPERRVVITGQGVVISLGQTI
EQFYSSLLEGVSGISQIQKFDTTGYITTIAGEIKSLQLDPYVPKRWAKRVDDVIKYVYIAGKQALESA
GLPIEAAGLAGAGLDPALCGVLIGTAMAGMTSFAAGVEALTRGGVRKMNPFCIPFSISNMGGAMLAMD
IGFMGPNYSISTACAIGNYCILGAADHIRRGDANVMLAGGADAAIIPSGIGGFIACKALSKRNDEPER
ASRPWDADRDGFVMGEGAGVLVLEELEHAKRRGATILAELVGGAATSDAHHMTEPDPQGRGVRLCLER
ALERARLAPERVGYVNAHGTSTPAGDVAEYRAIRAVIPQDSLRINSTKSMIGHLLGGAGAVEAVAAIQ
ALRIGWLHPNLNLENPAPGVDPVVLVGPRKERAEDLDVVLSNSFGFGGHNSCVIFRKYDEMDYKDHDG
DYKDHDIDYKDDDDK
Codon optimized M. polymorpha FAE3 (GenBank Accession No. AAP74370)
SEQ ID NO: 19
ATGgactcccgcgcccagaaccgcgacggcggcgaggacgtgaagcaggagctgctgtccgccggcga
cgacggcaaggtgccctgccccaccgtggccatcggcatccgccagcgcctgcccgacttcctgcagt
ccgtgaacatgaagtacgtgaagctgggctaccactacctgatcacccacgccatgttcctgctgacc
ctgcccgccttcttcctggtggccgccgagatcggccgcctgggccacgagcgcatctaccgcgagct
gtggacccacctgcacctgaacctggtgtccatcatggcctgctcctccgccctggtggccggcgcca
ccctgtacttcatgtcccgcccccgccccgtgtacctggtggagttcgcctgctaccgccccgacgag
cgcctgaaggtgtccaaggacttcttcctggacatgtcccgccgcaccggcctgttctcctcctcctc
catggacttccagaccaagatcacccagcgctccggcctgggcgacgagacctacctgccccccgcca
tcctggcctccccccccaacccctgcatgcgcgaggcccgcgaggaggccgccatggtgatgttcggc
gccctggacgagctgttcgagcagaccggcgtgaagcccaaggagatcggcgtgctggtggtgaactg
ctccctgttcaaccccaccccctccatgtccgccatgatcgtgaaccactaccacatgcgcggcaaca
tcaagtccctgaacctgggcggcatgggctgctccgccggcctgatctccatcgacctggcccgcgac
ctgctgcaggtgcacggcaacacctacgccgtggtggtgtccaccgagaacatcaccctgaactggta
cttcggcgacgaccgctccaagctgatgtccaactgcatcttccgcatgggcggcgccgccgtgctgc
tgtccaacaagcgccgcgagcgccgccgcgccaagtacgagctgctgcacaccgtgcgcacccacaag
ggcgccgacgacaagtgcttccgctgcgtgtaccaggaggaggactccaccggctccctgggcgtgtc
cctgtcccgcgagctgatggccgtggccggcaacgccctgaaggccaacatcaccaccctgggccccc
tggtgctgcccctgtccgagcagatcctgttcttcgcctccctggtggcccgcaagttcctgaacatg
aagatgaagccctacatccccgacttcaagctggccttcgagcacttctgcatccacgccggcggccg
cgccgtgctggacgagctggagaagaacctggacctgaccgagtggcacatggagccctcccgcatga
ccctgtaccgcttcggcaacacctcctcctcctccctgtggtacgagctggcctacaccgaggcccag
ggccgcgtgaagcgcggcgaccgcctgtggcagatcgccttcggctccggcttcaagtgcaactccgc
cgtgtggcgcgcgctgcgcaccgtgaagccccccgtgaacaacgcctggtccgacgtgatcgaccgct
tccccgtgaagctgccccagttcTGA
M. polymorpha FAE3 (GenBank Accession No. AAP74370)
SEQ ID NO: 20
MDSRAQNRDGGEDVKQELLSAGDDGKVPCPTVAIGIRQRLPDFLQSVNMKYVKLGYHYLITHAMFLLT
LPAFFLVAAEIGRLGHERIYRELWTHLHLNLVSIMACSSALVAGATLYFMSRPRPVYLVEFACYRPDE
RLKVSKDFFLDMSRRTGLFSSSSMDFQTKITQRSGLGDETYLPPAILASPPNPCMREAREEAAMVMFG
ALDELFEQTGVKPKEIGVLVVNCSLFNPIPSMSAMIVNHYHMRGNIKSLNLGGMGCSAGLISIDLARD
LLQVHGNIYAVVVSTENITLNWYFGDDRSKLMSNCIFRMGGAAVLLSNKRRERRRAKYELLHIVRTHK
GADDKCFRCVYQEEDSIGSLGVSLSRELMAVAGNALKANITTLGPLVLPLSEQILFFASLVARKFLNM
KMKPYIPDFKLAFEHFCIHAGGRAVLDELEKNLDLTEWHMEPSRMTLYRFGNISSSSLWYELAYTEAQ
GRVKRGDRLWQIAFGSGFKCNSAVWRALRIVKPPVNNAWSDVIDRFPVKLPQF
Trypanosoma brucei ELO3 (GenBank Accession No. AAX70673)
SEQ ID NO: 21

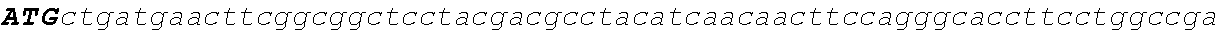

gtggatgctggaccacccctccgtgccctacatcgccggcgtgatgtacctgatcctggtgctgtacg
tgcccaagtccatcatggcctcccagccccccctgaacctgcgcgccgccaacatcgtgtggaacctg
ttcctgaccctgttctccatgtgcggcgcctactacaccgtgccctacctggtgaaggccttcatgaa
ccccgagatcgtgatggccgcctccggcatcaagctggacgccaacacctcccccatcatcacccact
ccggcttctacaccaccacctgcgccctggccgactccttctacttcaacggcgacgtgggcttctgg
gtggccctgttcgccctgtccaagatccccgagatgatcgacaccgccttcctggtgttccagaagaa
gcccgtgatcttcctgcactggtaccaccacctgaccgtgatgctgttctgctggttcgcctacgtgc
agaagatctcctccggcctgtggttcgcctccatgaactactccgtgcactccatcatgtacctgtac
tacttcgtgtgcgcctgcggccaccgccgcctggtgcgccccttcgcccccatcatcaccttcgtgca
gatcttccagatggtggtgggcaccatcgtggtgtgctacacctacaccgtgaagcacgtgctgggcc
gctcctgcaccgtgaccgacttctccctgcacaccggcctggtgatgtacgtgtcctacctgctgctg
ttctcccagctgttctaccgctcctacctgtccccccgcgacaaggcctccatcccccacgtggccgc

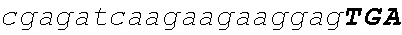

Trypanosoma brucei ELO3 (GenBank Accession No. AAX70673)
SEQ ID NO: 22
MYPTHRDLILNNYSDIYRSPTCHYHTWHILIHTPINELLFPNLPRECDFGYDIPYFRGQIDVFDGWSM
IHFISSNWCIPITVCLCYIMMIAGLKKYMGPRDGGRAPIQAKNYIIAWNLFLSFFSFAGVYYTVPYHL
FDPENGLFAQGFYSTVCNNGAYYGNGNVGFFVWLFIYSKIFELVDIFFLLIRKNPVIFLHWYHHLTVL
LYCWHAYSVRIGIGIWFATMNYSVHSVMYLYFAMTQYGPSTKKFAKKFSKFITTIQILQMVVGIIVTF
AAMLYVTFDVPCYTSLANSVLGLMMYASYFVLFVQLYVSHYVSPKHVKQE
Codon optimized Saccharomyces cerevisiae ELO1 (GenBank Accession No.
P39540)
SEQ ID NO: 23

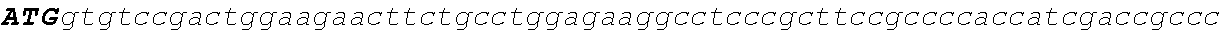

cttcttcaacatctacctgtgggactacttcaaccgcgccgtgggctgggccaccgccggccgcttcc
agcccaaggacttcgagttcaccgtgggcaagcagcccctgtccgagccccgccccgtgctgctgttc
atcgccatgtactacgtggtgatcttcggcggccgctccctggtgaagtcctgcaagcccctgaagct
gcgcttcatctcccaggtgcacaacctgatgctgacctccgtgtccttcctgtggctgatcctgatgg
tggagcagatgctgcccatcgtgtaccgccacggcctgtacttcgccgtgtgcaacgtggagtcctgg
acccagcccatggagaccctgtactacctgaactacatgaccaagttcgtggagttcgccgacaccgt
gctgatggtgctgaagcaccgcaagctgaccttcctgcacacctaccaccacggcgccaccgccctgc
tgtgctacaaccagctggtgggctacaccgccgtgacctgggtgcccgtgaccctgaacctggccgtg
cacgtgctgatgtactggtactacttcctgtccgcctccggcatccgcgtgtggtggaaggcctgggt
gacccgcctgcagatcgtgcagttcatgctggacctgatcgtggtgtactacgtgctgtaccagaaga
tcgtggccgcctacttcaagaacgcctgcaccccccagtgcgaggactgcctgggctccatgaccgcc
atcgccgccggcgccgccatcctgacctcctacctgttcctgttcatctccttctacatcgaggtgta

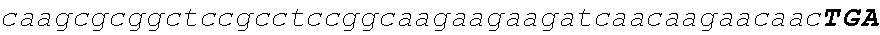

Saccharomyces cerevisiae ELO1 (GenBank Accession No. P39540)
SEQ ID NO: 24
MVSDWKNFCLEKASRFRPTIDRPFFNIYLWDYFNRAVGWATAGRFQPKDFEFTVGKQPLSEPRPVLLF
IAMYYVVIFGGRSLVKSCKPLKLRFISQVHNLMLTSVSFLWLILMVEQMLPIVYRHGLYFAVCNVESW
TQPMETLYYLNYMTKFVEFADTVLMVLKHRKLTFLHTYHHGATALLCYNQLVGYTAVTWVPVTLNLAV
HVLMYWYYFLSASGIRVWWKAWVTRLQIVQFMLDLIVVYYVLYQKIVAAYFKNACTPQCEDCLGSMTA
IAAGAAILTSYLFLFISFYIEVYKRGSASGKKKINKNN
23S rRNA for UTEX 1439, UTEX 1441, UTEX 1435, UTEX 1437 Prototheca
moriformis
SEQ ID NO: 25
TGTTGAAGAATGAGCCGGCGACTTAAAATAAATGGCAGGCTAAGAGAATTAATAACTCGAAACCTAAG
CGAAAGCAAGTCTTAATAGGGCGCTAATTTAACAAAACATTAAATAAAATCTAAAGTCATTTATTTTA
GACCCGAACCTGAGTGATCTAACCATGGTCAGGATGAAACTTGGGTGACACCAAGTGGAAGTCCGAAC
CGACCGATGTTGAAAAATCGGCGGATGAACTGTGGTTAGTGGTGAAATACCAGTCGAACTCAGAGCTA
GCTGGTTCTCCCCGAAATGCGTTGAGGCGCAGCAATATATCTCGTCTATCTAGGGGTAAAGCACTGTT
TCGGTGCGGGCTATGAAAATGGTACCAAATCGTGGCAAACTCTGAATACTAGAAATGACGATATATTA
GTGAGACTATGGGGGATAAGCTCCATAGTCGAGAGGGAAACAGCCCAGACCACCAGTTAAGGCCCCAA
AATGATAATGAAGTGGTAAAGGAGGTGAAAATGCAAATACAACCAGGAGGTTGGCTTAGAAGCAGCCA
TCCTTTAAAGAGTGCGTAATAGCTCACTG
Cu PSR23 LPAAT2-1
SEQ ID NO: 26
MAIAAAAVIFLFGLIFFASGLIINLFQALCFVLIRPLSKNAYRRINRVFAELLLSELLCLFDWWAGAK
LKLFTDPETFRLMGKEHALVIINHMTELDWMVGWVMGQHFGCLGSIISVAKKSTKFLPVLGWSMWFSE
YLYLERSWAKDKSTLKSHIERLIDYPLPFWLVIFVEGTRFTRTKLLAAQQYAVSSGLPVPRNVLIPRT
KGFVSCVSHMRSFVPAVYDVTVAFPKTSPPPTLLNLFEGQSIMLHVHIKRHAMKDLPESDDAVAEWCR
DKFVEKDALLDKHNAEDTFSGQEVCHSGSRQLKSLLVVISWVVVTTFGALKFLQWSSWKGKAFSAIGL
GIVTLLMHVLILSSQAERSNPAEVAQAKLKTGLSISKKVTDKEN
CuPSR23 LPAAT3-1
SEQ ID NO: 27
MAIAAAAVIVPLSLLFFVSGLIVNLVQAVCFVLIRPLSKNTYRRINRVVAELLWLELVWLIDWWAGVK
IKVFTDHETFHLMGKEHALVICNHKSDIDWLVGWVLGQRSGCLGSTLAVMKKSSKFLPVLGWSMWFSE
YLFLERSWAKDEITLKSGLNRLKDYPLPFWLALFVEGTRFTRAKLLAAQQYAASSGLPVPRNVLIPRT
KGFVSSVSHMRSFVPAIYDVTVAIPKTSPPPTLIRMFKGQSSVLHVHLKRHLMKDLPESDDAVAQWCR
DIFVEKDALLDKHNAEDTFSGQELQETGRPIKSLLVVISWAVLEVFGAVKFLQWSSLLSSWKGLAFSG
IGLGVITLLMHILILFSQSERSTPAKVAPAKPKNEGESSKTEMEKEK
Amino acid sequence for CuPSR23 LPPATx
SEQ ID NO: 28
MEIPPHCLCSPSPAPSQLYYKKKKHAILQTQTPYRYRVSPTCFAPPRLRKQHPYPLPVLCYPKLLHFS
QPRYPLVRSHLAEAGVAYRPGYELLGKIRGVCFYAVTAAVALLLFQCMLLLHPFVLLFDPFPRKAHHT
IAKLWSICSVSLFYKIHIKGLENLPPPHSPAVYVSNHQSFLDIYTLLTLGRTFKFISKTEIFLYPIIG
WAMYMLGTIPLKRLDSRSQLDTLKRCMDLIKKGASVFFFPEGTRSKDGKLGAFKKGAFSIAAKSKVPV
VPITLIGTGKIMPPGSELTVNPGTVQVIIHKPIEGSDAEAMCNEARATISHSLDD
cDNA sequence for CuPSR23 LPAATx coding region
SEQ ID NO: 29
ATGGAGATCCCGCCTCACTGTCTCTGTTCGCCTTCGCCTGCGCCTTCGCAATTGTATTACAAGAAGAA
GAAGCATGCCATTCTCCAAACTCAAACTCCCTATAGATATAGAGTTTCCCCGACATGCTTTGCCCCCC
CCCGATTGAGGAAGCAGCATCCTTACCCTCTCCCTGTCCTCTGCTATCCAAAACTCCTCCACTTCAGC
CAGCCTAGGTACCCTCTGGTTAGATCTCATTTGGCTGAAGCTGGTGTTGCTTATCGTCCAGGATACGA
ATTATTAGGAAAAATAAGGGGAGTGTGTTTCTATGCTGTCACTGCTGCCGTTGCCTTGCTTCTATTTC
AGTGCATGCTCCTCCTCCATCCCTTTGTGCTCCTCTTCGATCCATTTCCAAGAAAGGCTCACCATACC
ATCGCCAAACTCTGGTCTATCTGCTCTGTTTCTCTTTTTTACAAGATTCACATCAAGGGTTTGGAAAA
TCTTCCCCCACCCCACTCTCCTGCCGTCTATGTCTCTAATCATCAGAGTTTTCTCGACATCTATACTC
TCCTCACTCTCGGTAGAACCTTCAAGTTCATCAGCAAGACTGAGATCTTTCTCTATCCAATTATCGGT
TGGGCCATGTATATGTTGGGTACCATTCCTCTCAAGCGGTTGGACAGCAGAAGCCAATTGGACACTCT
TAAGCGATGTATGGATCTCATCAAGAAGGGAGCATCCGTCTTTTTCTTCCCAGAGGGAACACGAAGTA
AAGATGGGAAACTGGGTGCTTTCAAGAAAGGTGCATTCAGCATCGCAGCAAAAAGCAAGGTTCCTGTT
GTGCCGATCACCCTTATTGGAACTGGCAAGATTATGCCACCTGGGAGCGAACTTACTGTCAATCCAGG
AACTGTGCAAGTAATCATACATAAACCTATCGAAGGAAGTGATGCAGAAGCAATGTGCAATGAAGCTA
GAGCCACGATTTCTCACTCACTTGATGATTAA
cDNA sequence for CuPSR23 LPAAT 2-1 coding region
SEQ ID NO: 30
ATGGCGATTGCAGCGGCAGCTGTCATCTTCCTCTTCGGCCTTATCTTCTTCGCCTCCGGCCTCATAAT
CAATCTCTTCCAGGCGCTTTGCTTTGTCCTTATTCGGCCTCTTTCGAAAAACGCCTACMGGAGAATAA
ACAGAGTTTTTGCAGAATTGTTGTTGTCGGAGCTTTTATGCCTATTCGATTGGTGGGCTGGTGCTAAG
CTCAAATTATTTACCGACCCTGAAACCTTTCGCCTTATGGGCAAGGAACATGCTCTTGTCATAATTAA
TCACATGACTGAACTTGACTGGATGGTTGGATGGGTTATGGGTCAGCATTTTGGTTGCCTTGGGAGCA
TAATATCTGTTGCGAAGAAATCAACAAAATTTCTTCCGGTATTGGGGTGGTCAATGTGGTTTTCAGAG
TACCTATATCTTGAGAGAAGCTGGGCCAAGGATAAAAGTACATTAAAGTCACATATCGAGAGGCTGAT
AGACTACCCCCTGCCCTTCTGGTTGGTAATTTTTGTGGAAGGAACTCGGTTTACTCGGACAAAACTCT
TGGCAGCCCAGCAGTATGCTGTCTCATCTGGGCTACCAGTGCCGAGAAATGTTTTGATCCCACGTACT
AAGGGTTTTGTTTCATGTGTAAGTCACATGCGATCATTTGTTCCAGCAGTATATGATGTCACAGTGGC
ATTCCCTAAGACTTCACCTCCACCAACGTTGCTAAATCTTTTCGAGGGTCAGTCCATAATGCTTCACG
TTCACATCAAGCGACATGCAATGAAAGATTTACCAGAATCCGATGATGCAGTAGCAGAGTGGTGTAGA
GACAAATTTGTGGAAAAGGATGCTTTGTTGGACAAGCATAATGCTGAGGACACTTTCAGTGGTCAAGA
AGTTTGTCATAGCGGCAGCCGCCAGTTAAAGTCTCTTCTGGTGGTAATATCTTGGGTGGTTGTAACAA
CATTTGGGGCTCTAAAGTTCCTTCAGTGGTCATCATGGAAGGGGAAAGCATTTTCAGCTATCGGGCTG
GGCATCGTCACTCTACTTATGCACGTATTGATTCTATCCTCACAAGCAGAGCGGTCTAACCCTGCGGA
GGTGGCACAGGCAAAGCTAAAGACCGGGTTGTCGATCTCAAAGAAGGTAACGGACAAGGAAAACTAG
cDNA sequence for CuPSR23 LPAAx 3-1 coding region
SEQ ID NO: 31
ATGGCGATTGCTGCGGCAGCTGTCATCGTCCCGCTCAGCCTCCTCTTCTTCGTCTCCGGCCTCATCGT
CAATCTCGTACAGGCAGTTTGCTTTGTACTGATTAGGCCTCTGTCGAAAAACACTTACAGAAGAATAA
ACAGAGTGGTTGCAGAATTGTTGTGGTTGGAGTTGGTATGGCTGATTGATTGGTGGGCTGGTGTCAAG
ATAAAAGTATTCACGGATCATGAAACCTTTCACCTTATGGGCAAAGAACATGCTCTTGTCATTTGTAA
TCACAAGAGTGACATAGACTGGCTGGTTGGGTGGGTTCTGGGACAGCGGTCAGGTTGCCTTGGAAGCA
CATTAGCTGTTATGAAGAAATCATCAAAGTTTCTCCCGGTATTAGGGTGGTCAATGTGGTTCTCAGAG
TATCTATTCCTTGAAAGAAGCTGGGCCAAGGATGAAATTACATTAAAGTCAGGTTTGAATAGGCTGAA
AGACTATCCCTTACCCTTCTGGTTGGCACTTTTTGTGGAAGGAACTCGGTTCACTCGAGCAAAACTCT
TGGCAGCCCAGCAGTATGCTGCCTCTTCGGGGCTACCTGTGCCGAGAAATGTTCTGATCCCGCGTACT
AAGGGTTTTGTTTCTTCTGTGAGTCACATGCGATCATTTGTTCCAGCCATATATGATGTTACAGTGGC
AATCCCAAAGACGTCACCTCCACCAACATTGATAAGAATGTTCAAGGGACAGTCCTCAGTGCTTCACG
TCCACCTCAAGCGACACCTAATGAAAGATTTACCTGAATCAGATGATGCTGTTGCTCAGTGGTGCAGA
GATATATTCGTCGAGAAGGATGCTTTGTTGGATAAGCATAATGCTGAGGACACTTTCAGTGGCCAAGA
ACTTCAAGAAACTGGCCGCCCAATAAAGTCTCTTCTGGTTGTAATCTCTTGGGCGGTGTTGGAGGTAT
TTGGAGCTGTGAAGTTTCTTCAATGGTCATCGCTGTTATCATCATGGAAGGGACTTGCATTTTCGGGA
ATAGGACTGGGTGTCATCACGCTACTCATGCACATACTGATTTTATTCTCACAATCCGAGCGGTCTAC
CCCTGCAAAAGTGGCACCAGCAAAGCCAAAGAATGAGGGAGAGTCCTCCAAGACGGAAATGGAAAAGG
AAAAGTAG
cDNA sequence for CuPSR23 LPAATx coding region codon optimized for
Prototheca moriformis
SEQ ID NO: 32
ATGgagatccccccccactgcctgtgctccccctcccccgccccctcccagctgtactacaagaagaa
gaagcacgccatcctgcagacccagaccccctaccgctaccgcgtgtcccccacctgcttcgcccccc
cccgcctgcgcaagcagcacccctaccccctgcccgtgctgtgctaccccaagctgctgcacttctcc
cagccccgctaccccctggtgcgctcccacctggccgaggccggcgtggcctaccgccccggctacga
gctgctgggcaagatccgcggcgtgtgcttctacgccgtgaccgccgccgtggccctgctgctgttcc
agtgcatgctgctgctgcaccccttcgtgctgctgttcgaccccttcccccgcaaggcccaccacacc
atcgccaagctgtggtccatctgctccgtgtccctgttctacaagatccacatcaagggcctggagaa
cctgccccccccccactcccccgccgtgtacgtgtccaaccaccagtccttcctggacatctacaccc
tgctgaccctgggccgcaccttcaagttcatctccaagaccgagatcttcctgtaccccatcatcggc
tgggccatgtacatgctgggcaccatccccctgaagcgcctggactcccgctcccagctggacaccct
gaagcgctgcatggacctgatcaagaagggcgcctccgtgttcttcttccccgagggcacccgctcca
aggacggcaagctgggcgccttcaagaagggcgccttctccatcgccgccaagtccaaggtgcccgtg
gtgcccatcaccctgatcggcaccggcaagatcatgccccccggctccgagctgaccgtgaaccccgg
caccgtgcaggtgatcatccacaagcccatcgagggctccgacgccgaggccatgtgcaacgaggccc
gcgccaccatctcccactccctggacgacTGA
cDNA sequence for CuPSR23 LPAAT 2-1 coding region codon optimized
for Prototheca moriformis
SEQ ID NO: 33
ATGgcgatcgcggccgcggcggtgatcttcctgttcggcctgatcttcttcgcctccggcctgatcat
caacctgttccaggcgctgtgcttcgtcctgatccgccccctgtccaagaacgcctaccgccgcatca
accgcgtgttcgcggagctgctgctgtccgagctgctgtgcctgttcgactggtgggcgggcgcgaag
ctgaagctgttcaccgaccccgagacgttccgcctgatgggcaaggagcacgccctggtcatcatcaa
ccacatgaccgagctggactggatggtgggctgggtgatgggccagcacttcggctgcctgggctcca
tcatctccgtcgccaagaagtccacgaagttcctgcccgtgctgggctggtccatgtggttctccgag
tacctgtacctggagcgctcctgggccaaggacaagtccaccctgaagtcccacatcgagcgcctgat
cgactaccccctgcccttctggctggtcatcttcgtcgagggcacccgcttcacgcgcacgaagctgc
tggcggcccagcagtacgcggtctcctccggcctgcccgtcccccgcaacgtcctgatcccccgcacg
aagggcttcgtctcctgcgtgtcccacatgcgctccttcgtccccgcggtgtacgacgtcacggtggc
gttccccaagacgtcccccccccccacgctgctgaacctgttcgagggccagtccatcatgctgcacg
tgcacatcaagcgccacgccatgaaggacctgcccgagtccgacgacgccgtcgcggagtggtgccgc
gacaagttcgtcgagaaggacgccctgctggacaagcacaacgcggaggacacgttctccggccagga
ggtgtgccactccggctcccgccagctgaagtccctgctggtcgtgatctcctgggtcgtggtgacga
cgttcggcgccctgaagttcctgcagtggtcctcctggaagggcaaggcgttctccgccatcggcctg
ggcatcgtcaccctgctgatgcacgtgctgatcctgtcctcccaggccgagcgctccaaccccgccga
ggtggcccaggccaagctgaagaccggcctgtccatctccaagaaggtgacggacaaggagaacTGA
cDNA sequence for CuPSR23 LPAAx 3-1 coding region codon optimized
for Prototheca moriformis
SEQ ID NO: 34
ATGgccatcgcggcggccgcggtgatcgtgcccctgtccctgctgttcttcgtgtccggcctgatcgt
caacctggtgcaggccgtctgcttcgtcctgatccgccccctgtccaagaacacgtaccgccgcatca
accgcgtggtcgcggagctgctgtggctggagctggtgtggctgatcgactggtgggcgggcgtgaag
atcaaggtcttcacggaccacgagacgttccacctgatgggcaaggagcacgccctggtcatctgcaa
ccacaagtccgacatcgactggctggtcggctgggtcctgggccagcgctccggctgcctgggctcca
ccctggcggtcatgaagaagtcctccaagttcctgcccgtcctgggctggtccatgtggttctccgag
tacctgttcctggagcgctcctgggccaaggacgagatcacgctgaagtccggcctgaaccgcctgaa
ggactaccccctgcccttctggctggcgctgttcgtggagggcacgcgcttcacccgcgcgaagctgc
tggcggcgcagcagtacgccgcgtcctccggcctgcccgtgccccgcaacgtgctgatcccccgcacg
aagggcttcgtgtcctccgtgtcccacatgcgctccttcgtgcccgcgatctacgacgtcaccgtggc
catccccaagacgtcccccccccccacgctgatccgcatgttcaagggccagtcctccgtgctgcacg
tgcacctgaagcgccacctgatgaaggacctgcccgagtccgacgacgccgtcgcgcagtggtgccgc
gacatcttcgtggagaaggacgcgctgctggacaagcacaacgccgaggacaccttctccggccagga
gctgcaggagaccggccgccccatcaagtccctgctggtcgtcatctcctgggccgtcctggaggtgt
tcggcgccgtcaagttcctgcagtggtcctccctgctgtcctcctggaagggcctggcgttctccggc
atcggcctgggcgtgatcaccctgctgatgcacatcctgatcctgttctcccagtccgagcgctccac
ccccgccaaggtggcccccgcgaagcccaagaacgagggcgagtcctccaagaccgagatggagaagg
agaagTGA
SEQ ID NO: 35

| | |
|---|---|
| gctcttcgcc gccgccactc ctgctcgagc gcgcccgcgc gtgcgccgcc agcgccttgg | 60 |
| ccttttcgcc gcgctcgtgc gcgtcgctga tgtccatcac caggtccatg aggtctgcct | 120 |
| tgcgccggct gagccactgc ttcgtccggg cggccaagag gagcatgagg gaggactcct | 180 |
| ggtccagggt cctgacgtgg tcgcggctct gggagcgggc cagcatcatc tggctctgcc | 240 |
| gcaccgaggc cgcctccaac tggtcctcca gcagccgcag ccgccgccga ccctggcaga | 300 |
| ggaagacagg tgaggggggt atgaattgta cagaacaacc acgagccttg tctaggcaga | 360 |
| atccctacca gtcatggctt tacctggatg acggcctgcg aacagctgtc cagcgaccct | 420 |
| cgctgccgcc gcttctcccg cacgcttctt tccagcaccg tgatggcgcg agccagcgcc | 480 |
| gcacgctggc gctgcgcttc gccgatctga ggacagtcgg ggaactctga tcagtctaaa | 540 |
| cccccttgcg cgttagtgtt gccatccttt gcagaccggt gagagccgac ttgttgtgcg | 600 |
| ccacccccca caccacctcc tcccagacca attctgtcac ctttttggcg aaggcatcgg | 660 |
| cctcggcctg cagagaggac agcagtgccc agccgctggg ggttggcgga tgcacgctca | 720 |
| ggtacccttt cttgcgctat gacacttcca gcaaaaggta gggcgggctg cgagacggct | 780 |
| tcccggcgct gcatgcaaca ccgatgatgc ttcgaccccc cgaagctcct tcggggctgc | 840 |
| atgggcgctc cgatgccgct ccagggcgag cgctgtttaa atagccaggc ccccgattgc | 900 |
| aaagacatta tagcgagcta ccaaagccat attcaaacac ctagatcact accacttcta | 960 |
| cacaggccac tcgagcttgt gatcgcactc cgctaagggg gcgcctcttc ctcttcgttt | 1020 |
| cagtcacaac ccgcaaactc tagaatatca atgctgctgc aggccttcct gttcctgctg | 1080 |
| gccggcttcg ccgccaagat cagcgcctcc atgacgaacg agacgtccga ccgccccctg | 1140 |
| gtgcacttca cccccaacaa gggctggatg aacgacccca acggcctgtg gtacgacgag | 1200 |
| aaggacgcca agtggcacct gtacttccag tacaacccga acgacaccgt ctgggggacg | 1260 |
| cccttgttct ggggccacgc cacgtccgac gacctgacca actgggagga ccagcccatc | 1320 |
| aacaacacct ccggcttctt caacgacacc atcgacccgc gccagcgctg cgtggccatc | 1440 |
| tggacctaca acaccccgga gtccgaggag cagtacatct cctacagcct ggacggcggc | 1500 |
| tacaccttca ccgagtacca gaagaacccc gtgctggccg ccaactccac ccagttccgc | 1560 |
| gacccgaagg tcttctggta cgagccctcc cagaagtgga tcatgaccgc ggccaagtcc | 1620 |
| caggactaca agatcgagat ctactcctcc gacgacctga agtcctggaa gctggagtcc | 1680 |
| gcgttcgcca acgagggctt cctcggctac cagtacgagt gccccggcct gatcgaggtc | 1740 |
| cccaccgagc aggaccccag caagtcctac tgggtgatgt tcatctccat caaccccggc | 1800 |
| gccccggccg gcggctcctt caaccagtac ttcgtcggca gcttcaacgg cacccacttc | 1860 |
| gaggccttcg acaaccagtc ccgcgtggtg gacttcggca aggactacta cgccctgcag | 1920 |
| accttcttca acaccgaccc gacctacggg agcgccctgg gcatcgcgtg ggcctccaac | 1980 |
| tgggagtact ccgccttcgt gcccaccaac ccctggcgct cctccatgtc cctcgtgcgc | 2040 |
| aagttctccc tcaacaccga gtaccaggcc aacccggaga cggagctgat caacctgaag | 2100 |
| gccgagccga tcctgaacat cagcaacgcc ggcccctgga gccggttcgc caccaacacc | 2160 |
| acgttgacga aggccaacag ctacaacgtc gacctgtcca acagcaccgg caccctggag | 2220 |
| ttcgagctgg tgtacgccgt caacaccacc cagacgatct ccaagtccgt gttcgcggac | 2280 |
| ctctccctct ggttcaaggg cctggaggac cccgaggagt acctccgcat gggcttcgag | 2340 |
| gtgtccgcgt cctccttctt cctggaccgc gggaacagca aggtgaagtt cgtgaaggag | 2400 |
| aacccctact tcaccaaccg catgagcgtg aacaaccagc ccttcaagag cgagaacgac | 2460 |
| ctgtcctact acaaggtgta cggcttgctg gaccagaaca tcctggagct gtacttcaac | 2520 |
| gacggcgacg tcgtgtccac caacacctac ttcatgacca ccgggaacgc cctgggctcc | 2580 |
| gtgaacatga cgacgggggt ggacaacctg ttctacatcg acaagttcca ggtgcgcgag | 2640 |
| gtcaagtgac aattggcagc agcagctcgg atagtatcga cacactctgg acgctggtcg | 2700 |
| tgtgatggac tgttgccgcc acacttgctg ccttgacctg tgaatatccc tgccgctttt | 2760 |
| atcaaacagc ctcagtgtgt ttgatcttgt gtgtacgcgc ttttgcgagt tgctagctgc | 2820 |
| ttgtgctatt tgcgaatacc acccccagca tccccttccc tcgtttcata tcgcttgcat | 2880 |
| cccaaccgca acttatctac gctgtcctgc tatccctcag cgctgctcct gctcctgctc | 2940 |
| actgcccctc gcacagcctt ggtttgggct ccgcctgtat tctcctggta ctgcaacctg | 3000 |
| taaaccagca ctgcaatgct gatgcacggg aagtagtggg atgggaacac aaatggagga | 3060 |
| tcccgcgtct cgaacagagc gcgcagagga acgctgaagg tctcgcctct gtcgcacctc | 3120 |
| agcgcggcat acaccacaat aaccacctga cgaatgcgct tggttcttcg tccattagcg | 3180 |
| aagcgtccgg ttcacacacg tgccacgttg gcgaggtggc aggtgacaat gatcggtgga | 3240 |
| gctgatggtc gaaacgttca cagcctaggg atatcgaatt cggccgacag gacgcgcgtc | 3300 |
| aaaggtgctg gtcgtgtatg ccctggccgg caggtcgttg ctgctgctgg ttagtgattc | 3360 |
| cgcaaccctg attttggcgt cttattttgg cgtggcaaac gctggcgccc gcgagccggg | 3420 |
| ccggcggcga tgcggtgccc cacggctgcc ggaatccaag ggaggcaaga gcgcccgggt | 3480 |
| cagttgaagg gctttacgcg caaggtacag ccgctcctgc aaggctgcgt ggtggaattg | 3540 |
| gacgtgcagg tcctgctgaa gttcctccac cgcctcacca gcggacaaag caccggtgta | 3600 |
| tcaggtccgt gtcatccact ctaaagaact cgactacgac ctactgatgg ccctagattc | 3660 |
| ttcatcaaaa acgcctgaga cacttgccca ggattgaaac tccctgaagg gaccaccagg | 3720 |
| ggccctgagt tgttccttcc ccccgtggcg agctgccagc caggctgtac ctgtgatcga | 3780 |
| ggctggcggg aaaataggct tcgtgtgctc aggtcatggg aggtgcagga cagctcatga | 3840 |
| aacgccaaca atcgcacaat tcatgtcaag ctaatcagct atttcctctt cacgagctgt | 3900 |
| aattgtccca aaattctggt ctaccggggg tgatccttcg tgtacgggcc cttccctcaa | 3960 |
| ccctaggtat gcgcgcatgc ggtcgccgcg caactcgcgc gagggccgag ggtttgggac | 4020 |
| gggccgtccc gaaatgcagt tgcacccgga tgcgtggcac cttttttgcg ataatttatg | 4080 |
| caatggactg ctctgcaaaa ttctggctct gtcgccaacc ctaggatcag cggcgtagga | 4140 |
| tttcgtaatc attcgtcctg atggggagct accgactacc ctaatatcag cccgactgcc | 4200 |
| tgacgccagc gtccactttt gtgcacacat tccattcgtg cccaagacat ttcattgtgg | 4260 |
| tgcgaagcgt ccccagttac gctcacctgt ttcccgacct ccttactgtt ctgtcgacag | 4320 |
| agcgggccca caggccggtc gcagccacta gtatgacctc catcaacgtg aagctgctgt | 4380 |
| accactacgt gatcaccaac ctgttcaacc tgtgcttctt ccccctgacc gccatcgtgg | 4440 |
| ccggcaaggc ctcccgcctg accatcgacg acctgcacca cctgtactac tcctacctgc | 4500 |
| agcacaacgt gatcaccatc gcccccctgt tcgccttcac cgtgttcggc tccatcctgt | 4560 |
| acatcgtgac ccgccccaag cccgtgtacc tggtggagta ctcctgctac ctgcccccca | 4620 |
| cccagtgccg ctcctccatc tccaaggtga tggacatctt ctaccaggtg cgcaaggccg | 4680 |
| accccttccg caacggcacc tgcgacgact cctcctggct ggacttcctg cgcaagatcc | 4740 |
| aggagcgctc cggcctgggc gacgagaccc acggccccga gggcctgctg caggtgcccc | 4800 |
| cccgcaagac cttcgccgcc gcccgcgagg agaccgagca ggtgatcgtg ggcgccctga | 4860 |
| agaacctgtt cgagaacacc aaggtgaacc ccaaggacat cggcatcctg gtggtgaact | 4920 |
| cctccatgtt caaccccacc ccctccctgt ccgccatggt ggtgaacacc ttcaagctgc | 4980 |
| gctccaacgt gcgctccttc aacctgggcg gcatgggctg ctccgccggc gtgatcgcca | 5040 |
| tcgacctggc caaggacctg ctgcacgtgc acaagaacac ctacgccctg gtggtgtcca | 5100 |
| ccgagaacat cacctacaac atctacgccg gcgacaaccg ctccatgatg gtgtccaact | 5160 |
| gcctgttccg cgtgggcggc gccgccatcc tgctgtccaa caagccccgc gaccgccgcc | 5220 |
| gctccaagta cgagctggtg cacaccgtgc gcacccacac cggcgccgac gacaagtcct | 5280 |
| tccgctgcgt gcagcagggc gacgacgaga acggcaagac cggcgtgtcc ctgtccaagg | 5340 |
| acatcaccga ggtggccggc cgcaccgtga agaagaacat cgccaccctg ggccccctga | 5400 |
| tcctgcccct gtccgagaag ctgctgttct tcgtgacctt catggccaag aagctgttca | 5460 |
| aggacaaggt gaagcactac tacgtgcccg acttcaagct ggccatcgac cacttctgca | 5520 |
| tccacgccgg cggccgcgcc gtgatcgacg tgctggagaa gaacctgggc ctggccccca | 5580 |
| tcgacgtgga ggcctcccgc tccaccctgc accgcttcgg caacacctcc tcctcctcca | 5640 |
| tctggtacga gctggcctac atcgaggcca agggccgcat gaagaagggc aacaaggtgt | 5700 |
| ggcagatcgc cctgggctcc ggcttcaagt gcaactccgc cgtgtgggtg gccctgtcca | 5760 |
| acgtgaaggc ctccaccaac tccccctggg agcactgcat cgaccgctac cccgtgaaga | 5820 |
| tcgactccga ctccgccaag tccgagaccc gcgcccagaa cggccgctcc tgacttaagg | 5880 |
| cagcagcagc tcggatagta tcgacacact ctggacgctg gtcgtgtgat ggactgttgc | 5940 |
| cgccacactt gctgccttga cctgtgaata tccctgccgc ttttatcaaa cagcctcagt | 6000 |
| gtgtttgatc ttgtgtgtac gcgcttttgc gagttgctag ctgcttgtgc tatttgcgaa | 6060 |
| taccaccccc agcatcccct tccctcgttt catatcgctt gcatcccaac cgcaacttat | 6120 |
| ctacgctgtc ctgctatccc tcagcgctgc tcctgctcct gctcactgcc cctcgcacag | 6180 |
| ccttggtttg ggctccgcct gtattctcct ggtactgcaa cctgtaaacc agcactgcaa | 6240 |
| tgctgatgca cgggaagtag tgggatggga acacaaatgg aaagcttaat taagagctct | 6300 |
| tgttttccag aaggagttgc tccttgagcc tttcattctc agcctcgata acctccaaag | 6360 |
| ccgctctaat tgtggagggg gttcgaattt aaaagcttgg aatgttggtt cgtgcgtctg | 6420 |
| gaacaagccc agacttgttg ctcactggga aaaggaccat cagctccaaa aaacttgccg | 6480 |
| ctcaaaccgc gtacctctgc tttcgcgcaa tctgccctgt tgaaatcgcc accacattca | 6540 |
| tattgtgacg cttgagcagt ctgtaattgc ctcagaatgt ggaatcatct gccccctgtg | 6600 |
| cgagcccatg ccaggcatgt cgcgggcgag gacacccgcc actcgtacag cagaccatta | 6660 |
| tgctacctca caatagttca taacagtgac catatttctc gaagctcccc aacgagcacc | 6720 |
| tccatgctct gagtggccac cccccggccc tggtgcttgc ggagggcagg tcaaccggca | 6780 |
| tggggctacc gaaatccccg accggatccc accacccccg cgatgggaag aatctctccc | 6840 |
| cgggatgtgg gcccaccacc agcacaacct gctggcccag gcgagcgtca aaccatacca | 6900 |
| cacaaatatc cttggcatcg gccctgaatt ccttctgccg ctctgctacc cggtgcttct | 6960 |
| gtccgaagca ggggttgcta gggatcgctc cgagtccgca aacccttgtc gcgtggcggg | 7020 |
| gcttgttcga gcttgaagag c | 7041 |

SEQ ID NO: 36

| | |
|---|---|
| actagtatga cctccatcaa cgtgaagctg ctgtaccact acgtgatcac caacttcttc | 60 |
| aacctgtgct tcttccccct gaccgccatc ctggccggca aggcctcccg cctgaccacc | 120 |
| aacgacctgc accacttcta ctcctacctg cagcacaacc tgatcaccct gaccctgctg | 180 |
| ttcgccttca ccgtgttcgg ctccgtgctg tacttcgtga cccgccccaa gcccgtgtac | 240 |
| ctggtggact actcctgcta cctgcccccc cagcacctgt ccgccggcat ctccaagacc | 300 |
| atggagatct tctaccagat ccgcaagtcc gaccccctgc gcaacgtggc cctggacgac | 360 |
| tcctcctccc tggacttcct gcgcaagatc caggagcgct ccggcctggg cgacgagacc | 420 |
| tacggccccg agggcctgtt cgagatcccc ccccgcaaga acctggcctc cgcccgcgag | 480 |
| gagaccgagc aggtgatcaa cggcgccctg aagaacctgt tcgagaacac caaggtgaac | 540 |
| cccaaggaga tcggcatcct ggtggtgaac tcctccatgt tcaaccccac cccctccctg | 600 |
| tccgccatgg tggtgaacac cttcaagctg cgctccaaca tcaagtcctt caacctgggc | 660 |
| ggcatgggct gctccgccgg cgtgatcgcc atcgacctgg ccaaggacct gctgcacgtg | 720 |
| cacaagaaca cctacgccct ggtggtgtcc accgagaaca tcacccagaa catctacacc | 780 |
| ggcgacaacc gctccatgat ggtgtccaac tgcctgttcc gcgtgggcgg cgccgccatc | 840 |
| ctgctgtcca acaagcccgg cgaccgccgc cgctccaagt accgcctggc ccacaccgtg | 900 |
| cgcacccaca ccggcgccga cgacaagtcc ttcggctgcg tgcgccagga ggaggacgac | 960 |
| tccggcaaga ccggcgtgtc cctgtccaag gacatcaccg gcgtggccgg catcaccgtg | 1020 |
| cagaagaaca tcaccaccct gggccccctg gtgctgcccc tgtccgagaa gatcctgttc | 1080 |
| gtggtgacct tcgtggccaa gaagctgctg aaggacaaga tcaagcacta ctacgtgccc | 1140 |
| gacttcaagc tggccgtgga ccacttctgc atccacgccg gcggccgcgc cgtgatcgac | 1200 |
| gtgctggaga agaacctggg cctgtccccc atcgacgtgg aggcctcccg ctccaccctg | 1260 |
| caccgcttcg gcaacacctc ctcctcctcc atctggtacg agctggccta catcgaggcc | 1320 |
| aagggccgca tgaagaaggg caacaaggcc tggcagatcg ccgtgggctc cggcttcaag | 1380 |
| tgcaactccg ccgtgtgggt ggccctgcgc aacgtgaagg cctccgccaa ctccccctgg | 1440 |
| gagcactgca tccacaagta ccccgtgcag atgtactccg gctcctccaa gtccgagacc | 1500 |
| cgcgcccaga acggccgctc ctgacttaag | 1530 |

SEQ ID NO: 37

| | |
|---|---|
| actagtatga cctccatcaa cgtgaagctg ctgtaccact acgtgctgac caacttcttc | 60 |
| aacctgtgcc tgttccccct gaccgccttc cccgccggca aggcctccca gctgaccacc | 120 |
| aacgacctgc accacctgta ctcctacctg caccacaacc tgatcaccgt gaccctgctg | 180 |
| ttcgccttca ccgtgttcgg ctccatcctg tacatcgtga cccgccccaa gcccgtgtac | 240 |
| ctggtggact actcctgcta cctgcccccc cgccacctgt cctgcggcat ctcccgcgtg | 300 |
| atggagatct tctacgagat ccgcaagtcc gacccctccc gcgaggtgcc cttcgacgac | 360 |
| ccctcctccc tggagttcct gcgcaagatc caggagcgct ccggcctggg cgacgagacc | 420 |
| tacggccccc agggcctggt gcacgacatg cccctgcgca tgaacttcgc cgccgcccgc | 480 |
| gaggagaccg agcaggtgat caacggcgcc ctggagaagc tgttcgagaa caccaaggtg | 540 |
| aacccccgcg agatcggcat cctggtggtg aactcctcca tgttcaaccc caccccctcc | 600 |
| ctgtccgcca tggtggtgaa caccttcaag ctgcgctcca acatcaagtc cttctccctg | 660 |
| ggcggcatgg gctgctccgc cggcatcatc gccatcgacc tggccaagga cctgctgcac | 720 |
| gtgcacaaga acacctacgc cctggtggtg tccaccgaga acatcaccca ctccacctac | 780 |
| accggcgaca accgctccat gatggtgtcc aactgcctgt tccgcatggg cggcgccgcc | 840 |
| atcctgctgt ccaacaaggc cggcgaccgc cgccgctcca agtacaagct ggcccacacc | 900 |
| gtgcgcaccc acaccggcgc cgacgaccag tccttccgct gcgtgcgcca ggaggacgac | 960 |
| gaccgcggca agatcggcgt gtgcctgtcc aaggacatca ccgccgtggc cggcaagacc | 1020 |
| gtgaccaaga acatcgccac cctgggcccc ctggtgctgc ccctgtccga gaagttcctg | 1080 |
| tacgtggtgt ccctgatggc caagaagctg ttcaagaaca agatcaagca cacctacgtg | 1140 |
| cccgacttca agctggccat cgaccacttc tgcatccacg ccggcggccg cgccgtgatc | 1200 |
| gacgtgctgg agaagaacct ggccctgtcc cccgtggacg tggaggcctc ccgctccacc | 1260 |
| ctgcaccgct tcggcaacac ctcctcctcc tccatctggt acgagctggc ctacatcgag | 1320 |
| gccaagggcc gcatgaagaa gggcaacaag gtgtggcaga tcgccatcgg ctccggcttc | 1380 |
| aagtgcaact ccgccgtgtg ggtggccctg tgcaacgtga agccctccgt gaactccccc | 1440 |
| tgggagcact gcatcgaccg ctaccccgtg gagatcaact acggctcctc caagtccgag | 1500 |
| acccgcgccc agaacggccg ctcctgactt aag | 1533 |

SEQ ID NO: 38

| | |
|---|---|
| actagtatgt ccggcaccaa ggccacctcc gtgtccgtgc ccctgcccga cttcaagcag | 60 |
| tccgtgaacc tgaagtacgt gaagctgggc taccactact ccatcaccca cgccatgtac | 120 |
| ctgttcctga cccccctgct gctgatcatg tccgcccaga tctccacctt ctccatccag | 180 |
| gacttccacc acctgtacaa ccacctgatc ctgcacaacc tgtcctccct gatcctgtgc | 240 |
| atcgccctgc tgctgttcgt gctgaccctg tacttcctga cccgccccac ccccgtgtac | 300 |
| ctgctgaact tctcctgcta caagcccgac gccatccaca agtgcgaccg ccgccgcttc | 360 |
| acggacacca tccgcggcat gggcacctac accgaggaga acatcgagtt ccagcgcaag | 420 |
| gtgctggagc gctccggcat cggcgagtcc tcctacctgc cccccaccgt gttcaagatc | 480 |
| cccccccgcg tgtacgacgc cgaggagcgc gccgaggccg agatgctgat gttcggcgcc | 540 |
| gtggacggcc tgttcgagaa gatctccgtg aagcccaacc agatcggcgt gctggtggtg | 600 |
| aactgcggcc tgttcaaccc catcccctcc ctgtcctcca tgatcgtgaa ccgctacaag | 660 |
| atgcgcggca acgtgttctc ctacaacctg ggcggcatgg gctgctccgc cggcgtgatc | 720 |
| tccatcgacc tggccaagga cctgctgcag gtgcgcccca actcctacgc cctggtggtg | 780 |
| tccctggagt gcatctccaa gaacctgtac ctgggcgagc agcgctccat gctggtgtcc | 840 |
| aactgcctgt tccgcatggg cggcgccgcc atcctgctgt ccaacaagat gtccgaccgc | 900 |
| tggcgctcca agtaccgcct ggtgcacacc gtgcgcaccc acaagggcac cgaggacaac | 960 |
| tgcttctcct gcgtgacccg caaggaggac tccgacggca agatcggcat ctccctgtcc | 1020 |
| aagaacctga tggccgtggc cggcgacgcc ctgaagacca acatcaccac cctgggcccc | 1080 |
| ctggtgctgc ccatgtccga gcagctgctg ttcttcgcca ccctggtggg caagaaggtg | 1140 |
| ttcaagatga agctgcagcc ctacatcccc gacttcaagc tggccttcga gcacttctgc | 1200 |
| atccacgccg gcggccgcgc cgtgctggac gagctggaga agaacctgaa gctgtcctcc | 1260 |
| tggcacatgg agccctcccg catgtccctg taccgcttcg gcaacacctc ctcctcctcc | 1320 |
| ctgtggtacg agctggccta ctccgaggcc aagggccgca tcaagaaggg cgaccgcgtg | 1380 |
| tggcagatcg ccttcggctc cggcttcaag tgcaactccg ccgtgtggaa ggccctgcgc | 1440 |
| aacgtgaacc ccgccgagga gaagaacccc tggatggacg agatccacct gttccccgtg | 1500 |
| gaggtgcccc tgaactgact taag | 1524 |

SEQ ID NO: 39

| | |
|---|---|
| actagtatga cctccatcaa cgtgaagctg ctgtaccact acgtgatcac caacctgttc | 60 |
| aacctgtgct tcttccccct gaccgccatc gtggccggca aggcctacct gaccatcgac | 120 |
| gacctgcacc acctgtacta ctcctacctg cagcacaacc tgatcaccat cgcccccctg | 180 |
| ctggccttca ccgtgttcgg ctccgtgctg tacatcgcca cccgccccaa gcccgtgtac | 240 |
| ctggtggagt actcctgcta cctgcccccc acccactgcc gctcctccat ctccaaggtg | 300 |
| atggacatct tcttccaggt gcgcaaggcc gacccctccc gcaacggcac ctgcgacgac | 360 |
| tcctcctggc tggacttcct gcgcaagatc caggagcgct ccggcctggg cgacgagacc | 420 |
| cacggccccg agggcctgct gcaggtgccc ccccgcaaga ccttcgcccg cgcccgcgag | 480 |
| gagaccgagc aggtgatcat cggcgccctg gagaacctgt tcaagaacac caacgtgaac | 540 |
| cccaaggaca tcggcatcct ggtggtgaac tcctccatgt tcaaccccac cccctccctg | 600 |
| tccgccatgg tggtgaacac cttcaagctg cgctccaacg tgcgctcctt caacctgggc | 660 |
| ggcatgggct gctccgccgg cgtgatcgcc atcgacctgg ccaaggacct gctgcacgtg | 720 |
| cacaagaaca cctacgccct ggtggtgtcc accgagaaca tcacctacaa catctacgcc | 780 |
| ggcgacaacc gctccatgat ggtgtccaac tgcctgttcc gcgtgggcgg cgccgccatc | 840 |
| ctgctgtcca acaagccccg cgaccgccgc cgctccaagt acgagctggt gcacaccgtg | 900 |
| cgcacccaca ccggcgccga cgacaagtcc ttccgctgcg tgcagcaggg cgacgacgag | 960 |
| aacggccaga ccggcgtgtc cctgtccaag gacatcaccg acgtggccgg ccgcaccgtg | 1020 |
| aagaagaaca tcgccaccct gggccccctg atcctgcccc tgtccgagaa gctgctgttc | 1080 |
| ttcgtgacct tcatgggcaa gaagctgttc aaggacgaga tcaagcacta ctacgtgccc | 1140 |
| gacttcaagc tggccatcga ccacttctgc atccacgccg gcggcaaggc cgtgatcgac | 1200 |
| gtgctggaga agaacctggg cctggccccc atcgacgtgg aggcctcccg ctccaccctg | 1260 |
| caccgcttcg gcaacacctc ctcctcctcc atctggtacg agctggccta catcgagccc | 1320 |
| aagggccgca tgaagaaggg caacaaggtg tggcagatcg ccctgggctc cggcttcaag | 1380 |
| tgcaactccg ccgtgtgggt ggccctgaac aacgtgaagg cctccaccaa ctccccctgg | 1440 |
| gagcactgca tcgaccgcta ccccgtgaag atcgactccg actccggcaa gtccgagacc | 1500 |
| cgcgtgccca acggccgctc ctgacttaag | 1530 |

SEQ ID NO: 40

| | |
|---|---|
| actagtatgg agcgcaccaa ctccatcgag atggaccagg agcgcctgac cgccgagatg | 60 |
| gccttcaagg actcctcctc cgccgtgatc cgcatccgcc gccgcctgcc cgacttcctg | 120 |
| acctccgtga agctgaagta cgtgaagctg ggcctgcaca actccttcaa cttcaccacc | 180 |
| ttcctgttcc tgctgatcat cctgcccctg accggcaccg tgctggtgca gctgaccggc | 240 |
| ctgaccttcg agaccttctc cgagctgtgg tacaaccacg ccgcccagct ggacggcgtg | 300 |
| acccgcctgg cctgcctggt gtccctgtgc ttcgtgctga tcatctacgt gaccaaccgc | 360 |
| tccaagcccg tgtacctggt ggacttctcc tgctacaagc ccgaggacga gcgcaagatg | 420 |
| tccgtggact ccttcctgaa gatgaccgag cagaacggcg ccttcaccga cgacaccgtg | 480 |
| cagttccagc agcgcatctc caaccgcgcc ggcctgggcg acgagaccta cctgccccgc | 540 |
| ggcatcacct ccaccccccc caagctgaac atgtccgagg cccgcgccga ggccgaggcc | 600 |
| gtgatgttcg gcgccctgga ctccctgttc gagaagaccg gcatcaagcc cgccgaggtg | 660 |
| ggcatcctga tcgtgtcctg ctccctgttc aaccccaccc cctccctgtc cgccatgatc | 720 |
| gtgaaccact acaagatgcg cgaggacatc aagtcctaca acctgggcgg catgggctgc | 780 |
| tccgccggcc tgatctccat cgacctggcc aacaacctgc tgaaggccaa ccccaactcc | 840 |
| tacgccgtgg tggtgtccac cgagaacatc accctgaact ggtacttcgg caacgaccgc | 900 |
| tccatgctgc tgtgcaactg catcttccgc atgggcggcg ccgccatcct gctgtccaac | 960 |
| cgccgccagg accgctccaa gtccaagtac gagctggtga acgtggtgcg cacccacaag | 1020 |
| ggctccgacg acaagaacta caactgcgtg taccagaagg aggacgagcg cggcaccatc | 1080 |
| ggcgtgtccc tggcccgcga gctgatgtcc gtggccggcg acgccctgaa gaccaacatc | 1140 |
| accaccctgg gccccatggt gctgcccctg tccggccagc tgatgttctc cgtgtccctg | 1200 |
| gtgaagcgca agctgctgaa gctgaaggtg aagccctaca tccccgactt caagctggcc | 1260 |
| ttcgagcact tctgcatcca cgccggcggc cgcgccgtgc tggacgaggt gcagaagaac | 1320 |
| ctggacctgg aggactggca catggagccc tcccgcatga ccctgcaccg cttcggcaac | 1380 |
| acctcctcct cctccctgtg gtacgagatg gcctacaccg aggccaaggg ccgcgtgaag | 1440 |
| gccggcgacc gcctgtggca gatcgccttc ggctccggct tcaagtgcaa ctccgccgtg | 1500 |
| tggaaggccc tgcgcgtggt gtccaccgag gagctgaccg gcaacgcctg ggccggctcc | 1560 |
| atcgagaact accccgtgaa gatcgtgcag tgacttaag | 1599 |

SEQ ID NO: 41

| | |
|---|---|
| gctcttcgga gtcactgtgc cactgagttc gactggtagc tgaatggagt cgctgctcca | 60 |
| ctaaacgaat tgtcagcacc gccagccggc cgaggacccg agtcatagcg agggtagtag | 120 |
| cgcgccatgg caccgaccag cctgcttgcc agtactggcg tctcttccgc ttctctgtgg | 180 |
| tcctctgcgc gctccagcgc gtgcgctttt ccggtggatc atgcggtccg tggcgcaccg | 240 |
| cagcggccgc tgcccatgca gcgccgctgc ttccgaacag tggcggtcag ggccgcaccc | 300 |
| gcggtagccg tccgtccgga acccgcccaa gagttttggg agcagcttga gccctgcaag | 360 |
| atggcggagg acaagcgcat cttcctggag gagcaccggt gcgtggaggt ccggggctga | 420 |
| ccggccgtcg cattcaacgt aatcaatcgc atgatgatca gaggacacga agtcttggtg | 480 |
| gcggtggcca gaaacactgt ccattgcaag ggcataggga tgcgttcctt cacctctcat | 540 |
| ttctcatttc tgaatccctc cctgctcact ctttctcctc ctccttcccg ttcacgcagc | 600 |
| attcggggta ccgcggtgag aatcgaaaat gcatcgtttc taggttcgga gacggtcaat | 660 |
| tccctgctcc ggcgaatctg tcggtcaagc tggccagtgg acaatgttgc tatggcagcc | 720 |
| cgcgcacatg ggcctcccga cgcggccatc aggagcccaa acagcgtgtc agggtatgtg | 780 |
| aaactcaaga ggtccctgct gggcactccg gccccactcc gggggcggga cgccaggcat | 840 |
| tcgcggtcgg tcccgcgcga cgagcgaaat gatgattcgg ttacgagacc aggacgtcgt | 900 |
| cgaggtcgag aggcagcctc ggacacgtct cgctagggca acgccccgag tccccgcgag | 960 |
| ggccgtaaac attgtttctg ggtgtcggag tgggcatttt gggcccgatc caatcgcctc | 1020 |
| atgccgctct cgtctggtcc tcacgttcgc gtacggcctg gatcccggaa agggcggatg | 1080 |
| cacgtggtgt tgccccgcca ttggcgccca cgtttcaaag tccccggcca gaaatgcaca | 1140 |
| ggaccggccc ggctcgcaca ggccatgctg aacgcccaga tttcgacagc aacaccatct | 1200 |
| agaataatcg caaccatccg cgttttgaac gaaacgaaac ggcgctgttt agcatgtttc | 1260 |
| cgacatcgtg ggggccgaag catgctccgg ggggaggaaa gcgtggcaca gcggtagccc | 1320 |
| attctgtgcc acacgccgac gaggaccaat ccccggcatc agccttcatc gacggctgcg | 1380 |
| ccgcacatat aaagccggac gcctaaccgg tttcgtggtt atgactagta tgttcgcgtt | 1440 |
| ctacttcctg acggcctgca tctccctgaa gggcgtgttc ggcgtctccc cctcctacaa | 1500 |
| cggcctgggc ctgacgcccc agatgggctg ggacaactgg aacacgttcg cctgcgacgt | 1560 |
| ctccgagcag ctgctgctgg acacggccga ccgcatctcc gacctgggcc tgaaggacat | 1620 |
| gggctacaag tacatcatcc tggacgactg ctggtcctcc ggccgcgact ccgacggctt | 1680 |
| cctggtcgcc gacgagcaga agttccccaa cggcatgggc cacgtcgccg accacctgca | 1740 |
| caacaactcc ttcctgttcg gcatgtactc ctccgcgggc gagtacacgt gcgccggcta | 1800 |
| ccccggctcc ctgggccgcg aggaggagga cgcccagttc ttcgcgaaca accgcgtgga | 1860 |
| ctacctgaag tacgacaact gctacaacaa gggccagttc ggcacgcccg agatctccta | 1920 |
| ccaccgctac aaggccatgt ccgacgccct gaacaagacg ggccgcccca tcttctactc | 1980 |
| cctgtgcaac tggggccagg acctgacctt ctactggggc tccggcatcg cgaactcctg | 2040 |
| gcgcatgtcc ggcgacgtca cggcggagtt cacgcgcccc gactcccgct gcccctgcga | 2100 |
| cggcgacgag tacgactgca agtacgccgg cttccactgc tccatcatga acatcctgaa | 2160 |
| caaggccgcc cccatgggcc agaacgcggg cgtcggcggc tggaacgacc tggacaacct | 2220 |
| ggaggtcggc gtcggcaacc tgacggacga cgaggagaag gcgcacttct ccatgtgggc | 2280 |
| catggtgaag tcccccctga tcatcggcgc gaacgtgaac aacctgaagg cctcctccta | 2340 |
| ctccatctac tcccaggcgt ccgtcatcgc catcaaccag gactccaacg gcatccccgc | 2400 |
| cacgcgcgtc tggcgctact acgtgtccga cacggacgag tacggccagg gcgagatcca | 2460 |
| gatgtggtcc ggccccctgg acaacggcga ccaggtcgtg gcgctgctga acggcggctc | 2520 |
| cgtgtcccgc cccatgaaca cgaccctgga ggagatcttc ttcgactcca acctgggctc | 2580 |
| caagaagctg acctccacct gggacatcta cgacctgtgg gcgaaccgcg tcgacaactc | 2640 |
| cacggcgtcc gccatcctgg gccgcaacaa gaccgccacc ggcatcctgt acaacgccac | 2700 |
| cgagcagtcc tacaaggacg gcctgtccaa gaacgacacc cgcctgttcg gccagaagat | 2760 |
| cggctccctg tcccccaacg cgatcctgaa cacgaccgtc cccgcccacg gcatcgcgtt | 2820 |
| ctaccgcctg cgcccctcct cctgatacgt agcagcagca gctcggatag tatcgacaca | 2880 |
| ctctggacgc tggtcgtgtg atggactgtt gccgccacac ttgctgcctt gacctgtgaa | 2940 |
| tatccctgcc gcttttatca aacagcctca gtgtgtttga tcttgtgtgt acgcgctttt | 3000 |
| gcgagttgct agctgcttgt gctatttgcg aataccaccc ccagcatccc cttccctcgt | 3060 |
| ttcatatcgc ttgcatccca accgcaactt atctacgctg tcctgctatc cctcagcgct | 3120 |
| gctcctgctc ctgctcactg cccctcgcac agccttggtt tgggctccgc ctgtattctc | 3180 |
| ctggtactgc aacctgtaaa ccagcactgc aatgctgatg cacgggaagt agtgggatgg | 3240 |
| gaacacaaat ggagatatcg cgaggggtct gcctgggcca gccgctccct ctaaacacgg | 3300 |
| gacgcgtggt ccaattcggg cttcgggacc ctttggcggt ttgaacgcca gggatggggc | 3360 |
| gcccgcgagc ctggggaccc cggcaacggc ttccccagag cctgccttgc aatctcgcgc | 3420 |
| gtcctctccc tcagcacgtg gcggttccac gtgtggtcgg gcttcccgga ctagctcgcg | 3480 |
| tcgtgaccta gcttaatgaa cccagccggg cctgtagcac cgcctaagag gttttgatta | 3540 |
| tttcattata ccaatctatt cgccactagt atggccatca agaccaaccg ccagcccgtg | 3600 |
| gagaagcccc ccttcaccat cggcaccctg cgcaaggcca tccccgccca ctgcttcgag | 3660 |
| cgctccgccc tgcgctcctc catgtacctg gccttcgaca tcgccgtgat gtccctgctg | 3720 |
| tacgtggcct ccacctacat cgaccccgcc cccgtgccca cctgggtgaa gtacggcgtg | 3780 |
| atgtggcccc tgtactggtt cttccagggc gccttcggca ccggcgtgtg ggtgtgcgcc | 3840 |
| cacgagtgcg gccaccaggc cttctcctcc tcccaggcca tcaacgacgg cgtgggcctg | 3900 |
| gtgttccact ccctgctgct ggtgccctac tactcctgga agcactccca ccgccgccac | 3960 |
| cactccaaca ccggctgcct ggacaaggac gaggtgttcg tgccccccca ccgcgccgtg | 4020 |
| gcccacgagg gcctggagtg ggaggagtgg ctgcccatcc gcatgggcaa ggtgctggtg | 4080 |
| accctgaccc tgggctggcc cctgtacctg atgttcaacg tggcctcccg cccctacccc | 4140 |
| cgcttcgcca accacttcga cccctggtcc cccatcttct ccaagcgcga gcgcatcgag | 4200 |
| gtggtgatct ccgacctggc cctggtggcc gtgctgtccg gcctgtccgt gctgggccgc | 4260 |
| accatgggct gggcctggct ggtgaagacc tacgtggtgc cctacctgat cgtgaacatg | 4320 |
| tggctggtgc tgatcaccct gctgcagcac acccaccccg ccctgcccca ctacttcgag | 4380 |
| aaggactggg actggctgcg cggcgccatg gccaccgtgg accgctccat gggccccccc | 4440 |
| ttcatggaca acatcctgca ccacatctcc gacacccacg tgctgcacca cctgttctcc | 4500 |
| accatccccc actaccacgc cgaggaggcc tccgccgcca tccgccccat cctgggcaag | 4560 |
| tactaccagt ccgactcccg ctgggtgggc cgcgccctgt gggaggactg gcgcgactgc | 4620 |
| cgctacgtgg tgcccgacgc ccccgaggac gactccgccc tgtggttcca caagtagatc | 4680 |
| gatcttaagg cagcagcagc tcggatagta tcgacacact ctggacgctg gtcgtgtgat | 4740 |
| ggactgttgc cgccacactt gctgccttga cctgtgaata tccctgccgc ttttatcaaa | 4800 |
| cagcctcagt gtgtttgatc ttgtgtgtac gcgcttttgc gagttgctag ctgcttgtgc | 4860 |
| tatttgcgaa taccaccccc agcatcccct tccctcgttt catatcgctt gcatcccaac | 4920 |
| cgcaacttat ctacgctgtc ctgctatccc tcagcgctgc tcctgctcct gctcactgcc | 4980 |
| cctcgcacag ccttggtttg ggctccgcct gtattctcct ggtactgcaa cctgtaaacc | 5040 |
| agcactgcaa tgctgatgca cgggaagtag tgggatggga acacaaatgg aaagcttaat | 5100 |
| taagagctct tgttttccag aaggagttgc tccttgagcc tttcattctc agcctcgata | 5160 |
| acctccaaag ccgctctaat tgtggagggg gttcgaattt aaaagcttgg aatgttggtt | 5220 |
| cgtgcgtctg gaacaagccc agacttgttg ctcactggga aaaggaccat cagctccaaa | 5280 |
| aaacttgccg ctcaaaccgc gtacctctgc tttcgcgcaa tctgccctgt tgaaatcgcc | 5340 |
| accacattca tattgtgacg cttgagcagt ctgtaattgc ctcagaatgt ggaatcatct | 5400 |
| gccccctgtg cgagcccatg ccaggcatgt cgcgggcgag gacacccgcc actcgtacag | 5460 |
| cagaccatta tgctacctca caatagttca taacagtgac catatttctc gaagctcccc | 5520 |
| aacgagcacc tccatgctct gagtggccac cccccggccc tggtgcttgc ggagggcagg | 5580 |
| tcaaccggca tggggctacc gaaatccccg accggatccc accacccccg cgatgggaag | 5640 |
| aatctctccc cgggatgtgg gcccaccacc agcacaacct gctggcccag gcgagcgtca | 5700 |
| aaccatacca cacaaatatc cttggcatcg gccctgaatt ccttctgccg ctctgctacc | 5760 |
| cggtgcttct gtccgaagca ggggttgcta gggatcgctc cgagtccgca aacccttgtc | 5820 |
| gcgtggcggg gcttgttcga gcttgaagag c | 5851 |

SEQ ID NO: 42

| | |
|---|---|
| tacaacttat tacgtaacgg agcgtcgtgc gggagggagt gtgccgagcg gggagtcccg | 60 |
| gtctgtgcga ggcccggcag ctgacgctgg cgagccgtac gccccgaggg tccccctccc | 120 |
| ctgcaccctc ttccccttcc ctctgacggc cgcgcctgtt cttgcatgtt cagcgacgag | 180 |
| gatatc | 186 |

SEQ ID NO: 43

| | |
|---|---|
| gcgaggggtc tgcctgggcc agccgctccc tctgaacacg ggacgcgtgg tccaattcgg | 60 |
| gcttcgggac cctttggcgg tttgaacgcc tgggagaggg cgcccgcgag cctggggacc | 120 |
| ccggcaacgg cttccccaga gcctgccttg caatctcgcg cgtcctctcc ctcagcacgt | 180 |
| ggcggttcca cgtgtggtcg ggcgtcccgg actagctcac gtcgtgacct agcttaatga | 240 |
| acccagccgg gcctgcagca ccaccttaga ggttttgatt atttgattag accaatctat | 300 |
| tcacc | 305 |

SEQ ID NO: 44

| | |
|---|---|
| ggcgaataga ttggtataat gaaataatca aaacctctta ggcggtgcta caggcccggc | 60 |
| tgggttcatt aagctaggtc acgacgcgag ctagtccggg aagcccgacc acacgtggaa | 120 |
| ccgccacgtg cugagggaga ggacgcgcga gattgcaagg caggctctgg ggaagccgtt | 180 |
| gccggggtcc ccaggctcgc gggcgcccca tccctggcgt tcaaaccgcc aaagggtccc | 240 |
| gaagcccgaa ttggaccacg cgtcccgtgt ttagagggag cggctggccc aggcagaccc | 300 |
| ctcgc | 305 |

SEQ ID NO: 45

| | |
|---|---|
| ggtgaataga ttggtctaat caaataatca aaacctctaa ggtggtgctg caggcccggc | 60 |
| tgggttcatt aagctaggtc acgacgtgag ctagtccggg acgcccgacc acacgtggaa | 120 |
| ccgccacgtg ctgagggaga ggacgcgcga gattgcaagg caggctctgg ggaagccgtt | 180 |
| gccggggtcc ccaggctcgc gggcgccctc tcccaggcgt tcaaaccgcc aaagggtccc | 240 |
| gaagcccgaa ttggaccacg cgtcccgtgt tcagagggag cggctggccc aggcagaccc | 300 |
| ctcgc | 305 |

SEQ ID NO: 46

| | |
|---|---|
| gtgatgggtt ctttagacga tccagcccag gatcatgtgt tgcccacatg gagcctatcc | 60 |
| acgctggcct agaaggcaag cacatttcaa ggtgaaccca cgtccatgga gcgatggcgc | 120 |
| caatatctcg cctctagacc aagcggttct caccccaact gcgtcatttg tatgtatggc | 180 |
| tgcaaagttg tcggtacgat agaggccgcc aacctggcgg cgagggcgag gagctggttg | 240 |
| ccgatctgtg cccaagcatg tgtcggagct cggctgtctc ggcagcgagc tcctgtgcaa | 300 |
| ggggcttgca tcgagaatgt caggcgatag acactgcacg ttggggacac ggaggtgccc | 360 |
| ctgtggcgtg tcctggatgc cctcgggtcc gtcgcgagaa gctctggcga ccagcacccg | 420 |
| gccacaaccg cagcaggcgt tcacccacaa gaatcttcca gatcgtgatg cgcatgtatc | 480 |
| gtgacacgat tggcgaggtc cgcaggacgc acacggactc gtccactcat cagaactggt | 540 |
| cagggcaccc atctgcgtcc cttttcagga accacccacc gctgccaggc accttcgcca | 600 |
| gcggcggact ccacacagag aatgccttgc tgtgagagac catggccggc aagtgctgtc | 660 |
| ggatctgccc gcatacggtc agtccccagc acaaggaagc caagagtaca ggctgttggt | 720 |
| gtcgatggag gagtggccgt tcccacaagt agtgagcggc agctgctcaa cggcttcccc | 780 |
| ctgttcatct tggcaaagcc agtgacttcc tacaagtatg tgatgcagat cggcactgca | 840 |
| atctgtcggc atgcgtacag aacatcggct cgccagggca gcgttgctcg ctctggatga | 900 |
| gctgcttggg aggaatcatc ggcacacgcc cgtgccgtgc ccgcgccccg cgcccgtcgg | 960 |
| gaaaggcccc cggttaggac actgccgcgt cagccagtcg tgggatcgat cggacgtggc | 1020 |
| gaatcctcgc ccggacaccc tcatcacacc ccacatttcc ctgcaagcaa tcttgccgac | 1080 |
| aaaatagtca agatccattg ggtttaggga acacgtgcga gactgggcag ctgtatctgt | 1140 |
| ccttgccccg cgtcaaattc ctgggcgtga cgcagtcaca ggagaatcta ttagaccctg | 1200 |
| gacttgcagc tcagtcatgg gcgtgagtgg ctaaagcacc taggtcaggc gagtaccgcc | 1260 |
| ccttccccag gattcactct tctgcgattg acgttgagcc tgcatcgggc tgcttcgtca | 1320 |
| cc | 1322 |

SEQ ID NO: 47

| | |
|---|---|
| tcggagctaa agcagagact ggacaagact tgcgttcgca tactggtgac acagaatagc | 60 |
| tcccatctat tcatacgcct ttgggaaaag gaacgagcct tgtggcctct gcattgctgc | 120 |
| ctgctttgag gccgaggacg gtgcgggacg ctcagatcca tcagcgatcg ccccaccctc | 180 |
| agagcacctc cgatccaagg caatactatc aggcaaagtt tccaaattca aacattccaa | 240 |
| aatcacgcca gggactggat cacacacgca gatcagcgcc gttttgctct ttgcctacgg | 300 |
| gcgactgtgc cacttgtcga cccctggtga cgggagggac cacgcctgcg gttggcatcc | 360 |
| acttcgacgg acccagggac ggtttctcat gccaaacctg agatttgagc acccagatga | 420 |
| gcacattatg cgttttagga tgcctgagca gcgggcgtgc aggaatctgg tctcgccaga | 480 |
| ttcaccgaag atgcgcccat cggagcgagg cgagggcttt gtgaccacgc aaggcagtgt | 540 |
| gaggcaaaca catagggaca cctgcgtctt tcaatgcaca gacatctatg gtgcccatgt | 600 |
| atataaaatg ggctacttct gagtcaaacc aacgcaaact gcgctatggc aaggccggcc | 660 |
| aaggttggaa tcccggtctg tctggatttg agtttgtggg ggctatcacg tgacaatccc | 720 |
| tgggattggg cggcagcagc gcacggcctg ggtggcaatg gcgcactaat actgctgaaa | 780 |
| gcacggctct gcatcccttt ctcttgacct gcgattggtc cttttcgcaa gcgtgatcat | 840 |
| c | 841 |

SEQ ID NO:48

| | |
|---|---|
| tcggagctaa agcagaaact gaacaagact tgcgttcgca tacttgtgac actgaatagg | 60 |
| ttcaatctat tcatacgcct ttgggaaact gaacgagcct tgtggcctct gcattgctgc | 120 |
| ctgctttgag gccgaggacg gcgcggaacg cacagatcca tcagcgatcg ccccaccctc | 180 |
| agagtacatc cgatccaagg caatactatc aggcaaagtt tccaaattca aacattccaa | 240 |
| aattacgtca gggactggat cacacacgca gatcagcgcc gttttgctct ttgcctacgg | 300 |
| gcgactgtgc cacttgtcga cgcctggtga cgggagggac cacgcctgcg gttggcatcc | 360 |
| acttcgacgg acccagggac ggtctcacat gccaaacctg agatttgagc accaagatga | 420 |
| gcacattatg cgtttttgga tgcctgagca gcgggcgtgc aggaatctgg tctcgccaga | 480 |
| ttcaccgaag atgcggccat cggagcgagg cgagggctgt gtggccacgc caggcagtgt | 540 |
| gaggcaaaca cacagggaca tctgcttctt tcgatgcaca gacatctatg ttgcccgtgc | 600 |
| atataaaatg ggctacttct gaatcaaacc aacgcaaact tcgctatggc aaggccggcc | 660 |
| aaggttggaa tcccggtctg tctggatttg agtttgtggg ggctatcacg tgacaatccc | 720 |
| tgggattggg cggcagcagc gcacggcctg gatggcaatg gcgcactaat actgctgaaa | 780 |
| gcacggctct gcatcccttt ctcttgacct gcgattggtc cttttcgcaa gcgtgatcat | 840 |
| c | 841 |

SEQ ID NO: 49

| | |
|---|---|
| caccgatcac tccgtcgccg cccaagagaa atcaacctcg atggagggcg aggtggatca | 60 |
| gaggtattgg ttatcgttcg ttcttagtct caatcaatcg tacaccttgc agttgcccga | 120 |
| gtttctccac acatacagca cctcccgctc ccagcccatt cgagcgaccc aatccgggcg | 180 |
| atcccagcga tcgtcgtcgc ttcagtgctg accggtggaa agcaggagat ctcgggcgag | 240 |
| caggaccaca tccagcccag gatcttcgac tggctcagag ctgaccctca cgcggcacag | 300 |
| caaaagtagc acgcacgcgt tatgcaaact ggttacaacc tgtccaacag tgttgcgacg | 360 |
| ttgactggct acattgtctg tctgtcgcga gtgcgcctgg gcccttacgg tgggacactg | 420 |
| gaactccgcc ccgagtcgaa cacctagggc gacgcccgca gcttggcatg acagctctcc | 480 |
| ttgtgttcta aataccttgc gcgtgtggga ga | 512 |

SEQ ID NO: 50

| | |
|---|---|
| atccaccgat cactccgtcg ccgcccaaga gaattcaacc tcgatggagg gcaaggtgga | 60 |
| tcagaggtat tggttatcgt tcgctattag tctcaatcaa tcgtgcacct tgcagttgct | 120 |
| cgagtttctc cacacataca gcacctcccg ctcccagccc attcgagcga cccaatccgg | 180 |
| gcgatcccag cgatcgtcgt cgcttcagtg ctgaccggtg gaaagcagga gatctcgggc | 240 |
| gagcaggacc acatccagca caggatcttc gactggctca gagctgaccc tcacgcggca | 300 |
| cagcaaaagt agcccgcacg cgttatgcaa acaggttaca acctgtccaa cactgttgcg | 360 |
| acgttgactg gctacattgt ctgtctgtcg cgagtacgcc tggaccctta cggtgggaca | 420 |
| ctggaactcc gccccgagtc gaacacctag ggcgacgccc gcagcttggc atgacagctc | 480 |
| tccttgtatt ctaaatacct cgcgcgtgtg ggagaa | 516 |

SEQ ID NO: 51

| | |
|---|---|
| atgatgcgcg tgtacgacta tcaaggaaga aagaggactt aatttcttac cttctaacca | 60 |
| ccatattctt tttgctggat gcttgctcgt ctcgatgaca attgtgaacc tcttgtgtga | 120 |
| ccctgaccct gctgcaaggc tctccgaccg cacgcaaggc gcagccggcg cgtccggagg | 180 |
| cgatcggatc caatccagtc gtcctcccgc agcccgggca cgtttgccca tgcaggccct | 240 |
| tccacaccgc tcaagagact cccgaacacc gcccactcgg cactcgcttc ggctgccgag | 300 |
| tgcgcgtttg agtttgccct gccacagaag acacc | 335 |

SEQ ID NO: 52

| | |
|---|---|
| atgatgcgcg tgtacgacta tcaaggaaga aagaggactt aatttcttac cttctaacca | 60 |
| ccatattctt tttgctggat gcttgctcgt ctcgatgaca attgtgaacc tcttgtgtga | 120 |
| ccctgaccct gctgcaaggc tctccgaccg cacgcaaggc gcagccggcg cgtccggagg | 180 |
| cgatcggatc caatccagtc gtcctcccgc agcccgggca cgtttgccca tgcaggccct | 240 |
| tccacaccgc tcaagagact cccgaacacc gcccactcgg cactcgcttc ggctgccgag | 300 |
| tgcgcgtttg agtttgccct gccacaggag acatc | 335 |

SEQ ID NO: 53

| | |
|---|---|
| cccgggcgag ctgtacgcct acggagcgag gcctggtgtg accgttgcga tctcgccagc | 60 |
| agacgtcgcg gagcctcgtc ccaaaggccc tttctgatcg agcttgtcgt ccactggacg | 120 |
| ctttaagttg cgcgcgcgat gggataaccg agctgatctg cactcagatt ttggtttgtt | 180 |
| ttcgcgcatg gtgcagcgag gggaggtact acgctggggt acgagatcct ccggattccc | 240 |
| agaccgtgtt gccggcattt acccggtcat cgccagcgat tcgggacgac aaggccttat | 300 |
| cctgtgctga gacgctcgag cacgtttata aaattgtggg taccgcggta tgcacagcgt | 360 |
| tcaacacgcg ccacgccgaa attggttggt gggggagcac gtatgggact gacgtatggc | 420 |
| cagcagcgaa cactcaccga acaagtgcca atgtatacct tgcatcaatg atgctccggc | 480 |
| agcttcgatt gactgtctcg aaaaagtgtg agcaagcaga tcatgtggcc gctctgtcgc | 540 |
| gcagcacctg acgcattcga cacccacggc aatgcccagg ccagggaata gagagtaaga | 600 |
| caactcccat tgttcagcaa aacattgcac tgcagtgcct tcacaactat acaatgaatg | 660 |
| ggagggaata tgggctctgc atgggacagc ttagctggga cattcggcta ctgaacaaga | 720 |
| aaaccccacg agaaccaatt ggcgaaacct gccgggagga ggtgatcgtt tctgtaaatg | 780 |
| gcttacgcat tcccccccgg cggctcacga ggggtgtggt gaaccctgcc agctgatcaa | 840 |
| gtgcttgctg acgtcggcca gggaggtgta tgtgattggg ccgtggggcg tgagttatcc | 900 |
| taccgccgga cccgcgaagt cacatgacga atggccgtgc gggatgacga gagcacgact | 960 |
| cgctctttct tcgccggccc ggcttcatgg aggacaataa taaagggtgg ccaccggcaa | 1020 |
| cagccctcca tacctgaacc gattccagac ccaaacctct tgaattttga gggatccagt | 1080 |
| tcaccggtat agtcacg | 1097 |

SEQ ID NO: 54

| | |
|---|---|
| atccccgggc gagctgtacg cctacggagc gaggcctggt gtgaccgttg cgatctcgcc | 60 |
| agcagacgtc gcggagcctc gtcccaaagg ccctttctga tcgagcttgt cgtccactgg | 120 |
| acgctttaag ttgcgcgcgc gatgggataa ccgagctgat ctgcactcag attttggttt | 180 |
| gttttcgcgc atggtgcagc gaggggaggt actacgctgg ggtacgagat cctccggatt | 240 |
| cccagaccgt gttgccggca tttacccggt catcgccagc gattcgggac gacaaggcct | 300 |
| tatcctgtgc tgagacgctc gagcacgttt ataaaattgt ggtcaccgtg gtacgcacag | 360 |
| cgtccaacac gcgccacgcc gaaattcgtt ggtgggggag cacgtatcgg actgacgtat | 420 |
| ggccagcagc gaacactcac caaacaggtg ccaatgtata gcttgcatca atgatgctct | 480 |
| ggcagcttcg attgactgtc tcgaaaaagt gtgtgcaaac agattatgtg gccgctctgt | 540 |
| ggccgcgcag cacctgacgc actcgacacc cacggcaatg cccaggccaa ggaacagaga | 600 |
| gtaagacaac tcccattgtt cagtaaaaca ttgcactgca gtgccttcac aaacatacaa | 660 |
| cgaacgggag ggaatatggg cttcgaatgg gacagcttag ctgggacatt cggttactga | 720 |
| acaagaaaac cccacgagaa ccaactggcg aaacctgccg ggaggaggtg atcgtttttg | 780 |
| taaatggctt acgcattccc cccccggcgg ctcacggggg gtgtggtgaa ccctgccagc | 840 |
| tgatcaagtg cttgctgacg tcggccaggg aggtgtatgt gatttggccg tggggcgtga | 900 |
| gttatcctac cgccggaccc gcgaagtcac atgacgaatg gccgtgcggg atgacgagag | 960 |
| cagggctcgc tctttcttcg ccggcccggc ttcatggagg acaataataa agggtggcca | 1020 |
| ccggcaacag ccctccatac ctgaaccgat tccagaccca aacctcttga attttgaggg | 1080 |
| atccagttca ccggtatagt cacga | 1105 |

SEQ ID NO: 55

| | |
|---|---|
| gcgagtggtt ttgctgccgg gaagggagtg gggagcgtcg agcgagggac gcggcgctcg | 60 |
| aggcgcacgt cgtctgtcaa cgcgcgcggc cctcgcggcc cgcggcccca cccagctcta | 120 |
| atcatcgaaa actaagaggc tccacacgcc tgtcgtagaa tgcatgggat tcgccagtag | 180 |
| accacgatct gcgccgaaga agctggtcta cccgacgttt tttgttgctc ctttattctg | 240 |
| aatgatatga agatagtgtg cgcagtgcca cgcataggca tcaggagcaa gggaggacgg | 300 |
| gtcaacttga aagaaccaaa ccatccatcc gagaaatgcg catcatcttt gtagtaccat | 360 |
| caaacgcctt ggccaatgtc ttctgcatgg acaacacaac ctgctcctgg ccacacggtc | 420 |
| gacttggagc gccccatgcg cccaggtcgc cacgacccgc ggcccagcgc gcggcgattc | 480 |
| gcctcacgag atcccggcgg acccggcacg cccgcgggcc gacggtgcgc ttggcgatgc | 540 |
| tgctcattaa cccacggccg tcacccgatc cacatgctct ttttcaacac atccacattg | 600 |
| gaatagagct ctaccagggt gagtactgca ttctttgggg ctgggaggac cccactcgac | 660 |
| acctggtcct tcatcggccg aaagcccgaa cctgagcgct tccccgcccc gttcctcatc | 720 |
| cccgactttc cgatggccca ttgcagtttc aaac | 754 |

SEQ ID NO: 56

| | |
|---|---|
| atctgggtgg aggactggga gtaagatgta aggatattaa ttaaacattc tagtttgttg | 60 |
| atggcacaac agtcaatgca tttcagtcgt cttgctcctt ataacctatg cgtgtgccat | 120 |
| cgccggccat gcacctgtgg cgtggtaccg accatcgggg agaggcccga gattcggagg | 180 |
| tacctcccgc cctgggcgag cccttcacgt gacggcacaa gtcccttgca tcggcccgcg | 240 |
| agcacggaat acagagcccc gtgcccccca cgggccctca catcatccac tccattgttc | 300 |
| ttgccacacc gatcagca | 318 |

SEQ ID NO: 57

| | |
|---|---|
| tgggtggagg actgggaaga agatgtaagg atatcaattt aacattctag tttgttgatg | 60 |
| gcacaacagt cactgaatac cgggcgtctg gctgctaaaa tagccggage gtgtgccatc | 120 |
| gccggccatg catctgtggc gtggtaccga ccatcaggga gaggcccgag attcggaggt | 180 |
| acctcccgcc ccgggcgagc ccttcacgtg acggcacaag tcccttgcat cggcccgcga | 240 |
| gcacggaata cagagccccg tgctccccac gggccctcac atcatccact ccattgttct | 300 |
| tgccacaccg atcagc | 316 |

SEQ ID NO: 58

| | |
|---|---|
| ataacgaggc acaatgatcg atatttctat cgaacaactg tatttagccc tgtacgtacc | 60 |
| ccgctcttgg gccagcccgt ccgtgcttgc cttcggaaaa ttgcatggcg cctcatgcaa | 120 |
| actcgcgctc tcacagcaga tctcgcccag ctcccgggag agcaatcgcg ggtggggccc | 180 |
| ggggcgaatc caggacgcgc cccgcggggc cgctccactc gccagggcca atgggcggct | 240 |
| tatagtcctg gcatgggctc tgcatgcaca gtatcgcagt ttgggcgagg tgttgccccc | 300 |
| gcgatttcga atacgcgacg cccggtactc gtgcgagaac agggttcttg | 350 |

SEQ ID NO: 59

| | |
|---|---|
| atcgcgatgg tgcgcactcg tgcgcaatga atatggggtc acgcggtgga cgaacgcgga | 60 |
| gggggcctgg ccgaatctat gcttgcattc ctcagatcac tttctgccgg cggtccgggg | 120 |
| tttgcgcgtc gcgcaacgct ccgtctccct agccgctgcg caccgcgcgt gcgacgcgaa | 180 |
| ggtcattttc cagaacaacg accatggctt gtcttagcga tcgctcgaat gactgctagt | 240 |
| gagtcgtacg ctcgacccag tcgctcgcag gagaacgcgg caactgccga gcttcggctt | 300 |
| gccagtcgtg actcgtatgt gatcaggaat cattggcatt ggtagcatta taattcggct | 360 |
| tccgcgctgt ttatgggcat ggcaatgtct catgcagtcg accttagtca accaattctg | 420 |
| ggtggccagc tccgggcgac cgggctccgt gtcgccgggc accacctcct gccatgagta | 480 |
| acagggccgc cctctcctcc cgacgttggc ccactgaata ccgtgtcttg gggccctaca | 540 |
| tgatgggctg cctagtcggg cgggacgcgc aactgcccgc gcaatctggg acgtggtctg | 600 |
| aatcctccag gcgggtttcc ccgagaaaga aagggtgccg atttcaaagc agagccatgt | 660 |
| gccgggccct gtggcctgtg ttggcgccta tgtagtcacc ccccctcacc caattgtcgc | 720 |
| cagtttgcgc aatccataaa ctcaaaactg cagcttctga gctgcgctgt tcaagaacac | 780 |
| ctctggggtt tgctcacccg cgaggtcgac gcccagca | 818 |

SEQ ID NO: 60

| | |
|---|---|
| atcacgatgg tgcgcattcg tgcaaagtga atatggggtc acgcggtgga cgaacgcgga | 60 |
| gggggcatga ccgaatctag gctcgcattc ctcagatcac ttcatgccgg cggtccgggg | 120 |
| tttgcgcgtc gcgcaaggct acgtctccct agccgctgcg caccacgcgt gcgacgcgga | 180 |
| ggccatcttc cggagcaacg accatggatt gtcttagcga tcgcacgaat gagtgctagt | 240 |
| gagtcgtacg ctcgacccag tcgctcgcag gagaaggcgg cagctgccga gcttcggctt | 300 |
| accagtcgtg actcgtatgt gatcaggaat cattggcatt ggtagcatta taattcggct | 360 |
| tccgcgctgc gtatgggcat ggcaatgtct catgcagtcg atcttagtca accaattttg | 420 |
| ggtggccagg tccgggcgac cgggctccgt gtcgccgggc accacctcct gccaggagta | 480 |
| gcagggccgc cctctcgtcc cgacgttggc ccactgaata ccgtggcttc gagccctaca | 540 |
| tgatgggctg cctagtcggg cgggacgcgc aactgcccgc gcgatctggg ggctggtctg | 600 |
| aatccttcag gcgggtgtta cccgagaaag aaagggtgcc gatttcaaag cagacccatg | 660 |
| tgccgggccc tgtggcctgt gttggcgcct atgtagtcac cccccctcac ccaattgtcg | 720 |
| ccagtttgcg cactccataa actcaaaaca gcagcttctg agctgcgctg ttcaagaaca | 780 |
| cctctggggt ttgctcaccc gcgaggtcga cgcccagca | 819 |

SEQ ID NO: 61

| | |
|---|---|
| gctcttcgcc gccgccactc ctgctcgagc gcgcccgcgc gtgcgccgcc agcgccttgg | 60 |
| ccttttcgcc gcgctcgtgc gcgtcgctga tgtccatcac caggtccatg aggtctgcct | 120 |
| tgcgccggct gagccactgc ttcgtccggg cggccaagag gagcatgagg gaggactcct | 180 |
| ggtccagggt cctgacgtgg tcgcggctct gggagcgggc cagcatcatc tggctctgcc | 240 |
| gcaccgaggc cgcctccaac tggtcctcca gcagccgcag tcgccgccga ccctggcaga | 300 |
| ggaagacagg tgaggggggt atgaattgta cagaacaacc acgagccttg tctaggcaga | 360 |
| atccctacca gtcatggctt tacctggatg acggcctgcg aacagctgtc cagcgaccct | 420 |
| cgctgccgcc gcttctcccg cacgcttctt tccagcaccg tgatggcgcg agccagcgcc | 480 |
| gcacgctggc gctgcgcttc gccgatctga ggacagtcgg ggaactctga tcagtctaaa | 540 |
| cccccttgcg cgttagtgtt gccatccttt gcagaccggt gagagccgac ttgttgtgcg | 600 |
| ccacccccca caccacctcc tcccagacca attctgtcac ctttttggcg aaggcatcgg | 660 |
| cctcggcctg cagagaggac agcagtgccc agccgctggg ggttggcgga tgcacgctca | 720 |
| ggtacccttt cttgcgctat gacacttcca gcaaaaggta gggcgggctg cgagacggct | 780 |
| tcccggcgct gcatgcaaca ccgatgatgc ttcgaccccc cgaagctcct tcggggctgc | 840 |
| atgggcgctc cgatgccgct ccagggcgag cgctgtttaa atagccaggc ccccgattgc | 900 |
| aaagacatta tagcgagcta ccaaagccat attcaaacac ctagatcact accacttcta | 960 |
| cacaggccac tcgagcttgt gatcgcactc cgctaagggg gcgcctcttc ctcttcgttt | 1020 |
| cagtcacaac ccgcaaactc tagaatatca atgctgctgc aggccttcct gttcctgctg | 1080 |
| gccggcttcg ccgccaagat cagcgcctcc atgacgaacg agacgtccga ccgccccctg | 1140 |
| gtgcacttca cccccaacaa gggctggatg aacgacccca acggcctgtg gtacgacgag | 1200 |
| aaggacgcca agtggcacct gtacttccag tacaacccga acgacaccgt ctgggggacg | 1260 |
| cccttgttct ggggccacgc cacgtccgac gacctgacca actgggagga ccagcccatc | 1320 |
| gccatcgccc cgaagcgcaa cgactccggc gccttctccg gctccatggt ggtggactac | 1380 |
| aacaacacct ccggcttctt caacgacacc atcgacccgc gccagcgctg cgtggccatc | 1440 |
| tggacctaca acaccccgga gtccgaggag cagtacatct cctacagcct ggacggcggc | 1500 |
| tacaccttca ccgagtacca gaagaacccc gtgctggccg ccaactccac ccagttccgc | 1560 |
| gacccgaagg tcttctggta cgagccctcc cagaagtgga tcatgaccgc ggccaagtcc | 1620 |
| caggactaca agatcgagat ctactcctcc gacgacctga agtcctggaa gctggagtcc | 1680 |
| gcgttcgcca acgagggctt cctcggctac cagtacgagt gccccggcct gatcgaggtc | 1740 |
| cccaccgagc aggaccccag caagtcctac tgggtgatgt tcatctccat caaccccggc | 1800 |
| gccccggccg gcggctcctt caaccagtac ttcgtcggca gcttcaacgg cacccacttc | 1860 |
| gaggccttcg acaaccagtc ccgcgtggtg gacttcggca aggactacta cgccctgcag | 1920 |
| accttcttca acaccgaccc gacctacggg agcgccctgg gcatcgcgtg ggcctccaac | 1980 |
| tgggagtact ccgccttcgt gcccaccaac ccctggcgct cctccatgtc cctcgtgcgc | 2040 |
| aagttctccc tcaacaccga gtaccaggcc aacccggaga cggagctgat caacctgaag | 2100 |
| gccgagccga tcctgaacat cagcaacgcc ggcccctgga gccggttcgc caccaacacc | 2160 |
| acgttgacga aggccaacag ctacaacgtc gacctgtcca acagcaccgg caccctggag | 2220 |
| ttcgagctgg tgtacgccgt caacaccacc cagacgatct ccaagtccgt gttcgcggac | 2280 |
| ctctccctct ggttcaaggg cctggaggac cccgaggagt acctccgcat gggcttcgag | 2340 |
| gtgtccgcgt cctccttctt cctggaccgc gggaacagca aggtgaagtt cgtgaaggag | 2400 |
| aacccctact tcaccaaccg catgagcgtg aacaaccagc ccttcaagag cgagaacgac | 2460 |
| ctgtcctact acaaggtgta cggcttgctg gaccagaaca tcctggagct gtacttcaac | 2520 |
| gacggcgacg tcgtgtccac caacacctac ttcatgacca ccgggaacgc cctgggctcc | 2580 |
| gtgaacatga cgacgggggt ggacaacctg ttctacatcg acaagttcca ggtgcgcgag | 2640 |
| gtcaagtgac aattggcagc agcagctcgg atagtatcga cacactctgg acgctggtcg | 2700 |
| tgtgatggac tgttgccgcc acacttgctg ccttgacctg tgaatatccc tgccgctttt | 2760 |
| atcaaacagc ctcagtgtgt ttgatcttgt gtgtacgcgc ttttgcgagt tgctagctgc | 2820 |
| ttgtgctatt tgcgaatacc acccccagca tccccttccc tcgtttcata tcgcttgcat | 2880 |
| cccaaccgca acttatctac gctgtcctgc tatccctcag cgctgctcct gctcctgctc | 2940 |
| actgcccctc gcacagcctt ggtttgggct ccgcctgtat tctcctggta ctgcaacctg | 3000 |
| taaaccagca ctgcaatgct gatgcacggg aagtagtggg atgggaacac aaatggagga | 3060 |
| tcccgcgtct cgaacagagc gcgcagagga acgctgaagg tctcgcctct gtcgcacctc | 3120 |
| agcgcggcat acaccacaat aaccacctga cgaatgcgct tggttcttcg tccattagcg | 3180 |
| aagcgtccgg ttcacacacg tgccacgttg gcgaggtggc aggtgacaat gatcggtgga | 3240 |
| gctgatggtc gaaacgttca cagcctaggg atatcctgaa gaatgggagg caggtgttgt | 3300 |
| tgattatgag tgtgtaaaag aaaggggtag agagccgtcc tcagatccga ctactatgca | 3360 |
| tgattatgag tgtgtaaaag aaaggggtag agagccgtcc tcagatccga ctactatgca | 3360 |
| ggtagccgct cgcccatgcc cgcctggctg aatattgatg catgcccatc aaggcaggca | 3420 |
| ggcatttctg tgcacgcacc aagcccacaa tcttccacaa cacacagcat gtaccaacgc | 3480 |
| acgcgtaaaa gttggggtgc tgccagtgcg tcatgccagg catgatgtgc tcctgcacat | 3540 |
| ccgccatgat ctcctccatc gtctcgggtg tttccggcgc ctggtccggg agccgttccg | 3600 |
| ccagataccc agacgccacc tccgacctca cggggtactt ttcgagcgtc tgccggtagt | 3660 |
| cgacgatcgc gtccaccatg gagtagccga ggcgccggaa ctggcgtgac ggagggagga | 3720 |
| gagggaggag agagaggggg gggggggggg gggatgatta cacgccagtc tcacaacgca | 3780 |
| tgcaagaccc gtttgattat gagtacaatc atgcactact agatggatga gcgccaggca | 3840 |
| taaggcacac cgacgttgat ggcatgagca actcccgcat catatttcct attgtcctca | 3900 |
| cgccaagccg gtcaccatcc gcatgctcat attacagcgc acgcaccgct tcgtgatcca | 3960 |
| ccgggtgaac gtagtcctcg acggaaacat ctggctcggg cctcgtgctg gcactccctc | 4020 |
| ccatgccgac aacctttctg ctgtcaccac gacccacgat gcaacgcgac acgacccggt | 4080 |
| gggactgatc ggttcactgc acctgcatgc aattgtcaca agcgcatact ccaatcgtat | 4140 |
| ccgtttgatt tctgtgaaaa ctcgctcgac cgcccgcgtc ccgcaggcag cgatgacgtg | 4200 |
| tgcgtgacct gggtgtttcg tcgaaaggcc agcaacccca aatcgcaggc gatccggaga | 4260 |
| ttgggatctg atccgagctt ggaccagatc ccccacgatg cggcacggga actgcatcga | 4320 |
| ctcggcgcgg aacccagctt tcgtaaatgc cagattggtg tccgatacct tgatttgcca | 4380 |
| tcagcgaaac aagacttcag cagcgagcgt atttggcggg cgtgctacca gggttgcata | 4440 |
| cattgcccat ttctgtctgg accgctttac cggcgcagag ggtgagttga tggggttggc | 4500 |
| aggcatcgaa acgcgcgtgc atggtgtgtg tgtctgtttt cggctgcaca atttcaatag | 4560 |
| tcggatgggc gacggtagaa ttgggtgttg cgctcgcgtg catgcctcgc cccgtcgggt | 4620 |
| gtcatgaccg ggactggaat cccccctcgc gaccctcctg ctaacgctcc cgactctccc | 4680 |
| gcccgcgcgc aggatagact ctagttcaac caatcgacaa ctagtatggc caccgcatcc | 4740 |
| actttctcgg cgttcaatgc ccgctgcggc gacctgcgtc gctcggcggg ctccgggccc | 4800 |
| cggcgcccag cgaggcccct ccccgtgcgc gggcgcgcca tccccccccg catcatcgtg | 4860 |
| gtgtcctcct cctcctccaa ggtgaacccc ctgaagaccg aggccgtggt gtcctccggc | 4920 |
| ctggccgacc gcctgcgcct gggctccctg accgaggacg gcctgtccta caaggagaag | 4980 |
| ttcatcgtgc gctgctacga ggtgggcatc aacaagaccg ccaccgtgga gaccatcgcc | 5040 |
| aacctgctgc aggaggtggg ctgcaaccac gcccagtccg tgggctactc caccggcggc | 5100 |
| ttctccacca cccccaccat gcgcaagctg cgcctgatct gggtgaccgc ccgcatgcac | 5160 |
| atcgagatct acaagtaccc cgcctggtcc gacgtggtgg agatcgagtc ctggggccag | 5220 |
| ggcgagggca agatcggcac ccgccgcgac tggatcctgc gcgactacgc caccggccag | 5280 |
| gtgatcggcc gcgccacctc caagtgggtg atgatgaacc aggacacccg ccgcctgcag | 5340 |
| aaggtggacg tggacgtgcg cgacgagtac ctggtgcact gcccccgcga gctgcgcctg | 5400 |
| gccttccccg aggagaacaa ctcctccctg aagaagatct ccaagctgga ggacccctcc | 5460 |
| cagtactcca agctgggcct ggtgccccgc cgcgccgacc tggacatgaa ccagcacgtg | 5520 |
| aacaacgtga cctacatcgg ctgggtgctg gagtccatgc cccaggagat catcgacacc | 5580 |
| cacgagctgc agaccatcac cctggactac cgccgcgagt gccagcacga cgacgtggtg | 5640 |
| gactccctga cctcccccga gccctccgag gacgccgagg ccgtgttcaa ccacaacggc | 5700 |
| accaacggct ccgccaacgt gtccgccaac gaccacggct gccgcaactt cctgcacctg | 5760 |
| ctgcgcctgt ccggcaacgg cctggagatc aaccgcggcc gcaccgagtg gcgcaagaag | 5820 |
| cccacccgca tggactacaa ggaccacgac ggcgactaca aggaccacga catcgactac | 5880 |
| aaggacgacg acgacaagtg aatcgataga tctcttaagg cagcagcagc tcggatagta | 5940 |
| tcgacacact ctggacgctg gtcgtgcgat ggactgttgc cgccacactt gctgccttga | 6000 |
| cctgtgaata tccctgccgc ttttatcaaa cagcctcagt gtgtttgatc ttgtgtgtac | 6060 |
| gcgcttttgc gagttgctag ctgcttgtgc tatttgcgaa taccaccccc agcatcccct | 6120 |
| tccctcgttt catatcgctt gcatcccaac cgcaacttat ctacgctgtc ctgctatccc | 6180 |
| tcagcgctgc tcctgctcct gctcactgcc cctcgcacag ccttggtttg ggctccgcct | 6240 |
| gtattctcct ggtactgcaa cctgtaaacc agcactgcaa tgctgatgca cgggaagtag | 6300 |
| tgggatggga acacaaatgg aaagcttaat taagagctct tgttttccag aaggagttgc | 6360 |
| tccttgagcc tttcattctc agcctcgata acctccaaag ccgctctaat tgtggagggg | 6420 |
| gttcgaattt aaaagcttgg aatgttggtt cgtgcgtctg gaacaagccc agacttgttg | 6480 |
| ctcactggga aaaggaccat cagctccaaa aaacttgccg ctcaaaccgc gtacctctgc | 6540 |
| tttcgcgcaa tctgccctgt tgaaatcgcc accacattca tattgtgacg cttgagcagt | 6600 |
| ctgtaattgc ctcagaatgt ggaatcatct gccccctgtg cgagcccatg ccaggcatgt | 6660 |
| cgcgggcgag gacacccgcc actcgtacag cagaccatta tgctacctca caatagttca | 6720 |
| taacagtgac catatttctc gaagctcccc aacgagcacc tccatgctct gagtggccac | 6780 |
| cccccggccc tggtgcttgc ggagggcagg tcaaccggca tggggctacc gaaatccccg | 6840 |
| accggatccc accacccccg cgatgggaag aatctctccc cgggatgtgg gcccaccacc | 6900 |
| agcacaacct gctggcccag gcgagcgtca aaccatacca cacaaatatc cttggcatcg | 6960 |
| gccctgaatt ccttctgccg ctctgctacc cggtgcttct gtccgaagca ggggttgcta | 7020 |
| gggatcgctc cgagtccgca aacccttgtc gcgtggcggg gcttgttcga gcttgaagag | 7080 |
| c | 7081 |

SEQ ID NO: 62

| | |
|---|---|
| gctcttccca actcagataa taccaatacc cctccttctc ctcctcatcc attcagtacc | 60 |
| cccccccttc tcttcccaaa gcagcaagcg cgtggcttac agaagaacaa tcggcttccg | 120 |
| ccaaagtcgc cgagcactgc ccgacggcgg cgcgcccagc agcccgcttg gccacacagg | 180 |
| caacgaatac attcaatagg gggcctcgca gaatggaagg agcggtaaag ggtacaggag | 240 |
| cactgcgcac aaggggcctg tgcaggagtg actgactggg cgggcagacg gcgcaccgcg | 300 |
| ggcgcaggca agcagggaag attgaagcgg cagggaggag gatgctgatt gaggggggca | 360 |
| tcgcagtctc tcttggaccc gggataagga agcaaatatt cggccggttg ggttgtgtgt | 420 |
| gtgcacgttt tcttcttcag agtcgtgggt gtgcttccag ggaggatata agcagcagga | 480 |
| tcgaatcccg cgaccagcgt ttccccatcc agccaaccac cctgtcggta ccgcggtgag | 540 |
| aatcgaaaat gcatcgtttc taggttcgga gacggtcaat tccctgctcc ggcgaatctg | 600 |
| tcggtcaagc tggccagtgg acaatgttgc tatggcagcc cgcgcacatg ggcctcccga | 660 |
| cgcggccatc aggagcccaa acagcgtgtc agggtatgtg aaactcaaga ggtccctgct | 720 |
| gggcactccg gccccactcc gggggcggga cgccaggcat tcgcggtcgg tcccgcgcga | 780 |
| cgagcgaaat gatgattcgg ttacgagacc aggacgtcgt cgaggtcgag aggcagcctc | 840 |
| ggacacgtct cgctagggca acgccccgag tccccgcgag ggccgtaaac attgtttctg | 900 |
| ggtgtcggag tgggcatttt gggcccgatc caatcgcctc atgccgctct cgtctggtcc | 960 |
| tcacgttcgc gtacggcctg gatcccggaa agggcggatg cacgtggtgt tgccccgcca | 1020 |
| ttggcgccca cgtttcaaag tccccggcca gaaatgcaca ggaccggccc ggctcgcaca | 1080 |
| ggccatgctg aacgcccaga tttcgacagc aacaccatct agaataatcg caaccatccg | 1140 |
| cgttttgaac gaaacgaaac ggcgctgttt agcatgtttc cgacatcgcg ggggccgaag | 1200 |
| catgctccgg ggggaggaaa gcgtggcaca gcggtagccc attctgtgcc acacgccgac | 1260 |
| gaggaccaat ccccggcatc agccttcatc gacggctgcg ccgcacatat aaagccggac | 1320 |
| gcctaaccgg tttcgtggtt atgactagta tgttcgcgtt ctacttcctg acggcctgca | 1380 |
| tctccctgaa gggcgtgttc ggcgtctccc cctcctacaa cggcctgggc ctgacgcccc | 1440 |
| agatgggctg ggacaactgg aacacgttcg cctgcgacgt ctccgagcag ctgctgctgg | 1500 |
| acacggccga ccgcatctcc gacctgggcc tgaaggacat gggctacaag tacatcatcc | 1560 |
| tggacgactg ctggtcctcc ggccgcgact ccgacggctt cctggtcgcc gacgagcaga | 1620 |
| agttccccaa cggcatgggc cacgtcgccg accacctgca caacaactcc ttcctgttcg | 1680 |
| gcatgtactc ctccgcgggc gagtacacgt gcgccggcta ccccggctcc ctgggccgcg | 1740 |
| aggaggagga cgcccagttc ttcgcgaaca accgcgtgga ctacctgaag tacgacaact | 1800 |
| gctacaacaa gggccagttc ggcacgcccg agatctccta ccaccgctac aaggccatgt | 1860 |
| ccgacgccct gaacaagacg ggccgcccca tcttctactc cctgtgcaac tggggccagg | 1920 |
| acctgacctt ctactggggc tccggcatcg cgaactcctg gcgcatgtcc ggcgacgtca | 1980 |
| cggcggagtt cacgcgcccc gactcccgct gcccctgcga cggcgacgag tacgactgca | 2040 |
| agtacgccgg cttccactgc tccatcatga acatcctgaa caaggccgcc cccatgggcc | 2100 |
| agaacgcggg cgtcggcggc tggaacgacc tggacaacct ggaggtcggc gtcggcaacc | 2160 |
| tgacggacga cgaggagaag gcgcacttct ccatgtgggc catggtgaag tcccccctga | 2220 |
| tcatcggcgc gaacgtgaac aacctgaagg cctcctccta ctccatctac tcccaggcgt | 2280 |
| ccgtcatcgc catcaaccag gactccaacg gcatccccgc cacgcgcgtc tggcgctact | 2340 |
| acgtgtccga cacggacgag tacggccagg gcgagatcca gatgtggtcc ggccccctgg | 2400 |
| acaacggcga ccaggtcgtg gcgctgctga acggcggctc cgtgtcccgc cccatgaaca | 2460 |
| cgaccctgga ggagatcttc ttcgactcca acctgggctc caagaagctg acctccacct | 2520 |
| gggacatcta cgacctgtgg gcgaaccgcg tcgacaactc cacggcgtcc gccatcctgg | 2580 |
| gccgcaacaa gaccgccacc ggcatcctgt acaacgccac cgagcagtcc tacaaggacg | 2640 |
| gcctgtccaa gaacgacacc cgcctgttcg gccagaagat cggctccctg tcccccaacg | 2700 |
| cgatcctgaa cacgaccgtc cccgcccacg gcatcgcgtt ctaccgcctg cgcccctcct | 2760 |
| cctgatacaa cttattacgt attctgaccg gcgctgatgt ggcgcggacg ccgtcgtact | 2820 |
| ctttcagact ttactcttga ggaattgaac ctttctcgct tgctggcatg taaacattgg | 2880 |
| cgcaattaat tgtgtgatga agaaagggtg gcacaagatg gatcgcgaat gtacgagatc | 2940 |
| gacaacgatg gtgattgtta tgaggggcca aacctggctc aatcttgtcg catgtccggc | 3000 |
| gcaatgtgat ccagcggcgt gactctcgca acctggtagt gtgtgcgcac cgggtcgctt | 3060 |
| tgattaaaac tgatcgcatt gccatcccgt caactcacaa gcctactcta gctcccattg | 3120 |
| cgcactcggg cgcccggctc gatcaatgtt ctgagcggag ggcgaagcgt caggaaatcg | 3180 |
| tctcggcagc tggaagcgca tggaatgcgg agcggagatc gaatcaggat cccgcgtctc | 3240 |
| gaacagagcg cgcagaggaa cgctgaaggt ctcgcctctg tcgcacctca gcgcggcata | 3300 |
| caccacaata accacctgac gaatgcgctt ggttcttcgt ccattagcga agcgtccggt | 3360 |
| tcacacacgt gccacgttgg cgaggtggca ggtgacaatg atcggtggag ctgatggtcg | 3420 |
| aaacgttcac agcctagcat agcgactgct accccccgac catgtgccga ggcagaaatt | 3480 |
| atatacaaga agcagatcgc aattaggcac atcgctttgc attatccaca cactattcat | 3540 |
| cgctgctgcg gcaaggctgc agagtgtatt tttgtggccc aggagctgag tccgaagtcg | 3600 |
| acgcgacgag cggcgcagga tccgacccct agacgagctc tgtcattttc caagcacgca | 3660 |
| gctaaatgcg ctgagaccgg gtctaaatca tccgaaaagt gtcaaaatgg ccgattgggt | 3720 |
| tcgcctagga caatgcgctg cggattcgct cgagtccgct gccggccaaa aggcggtggt | 3780 |
| acaggaaggc gcacggggcc aaccctgcga agccgggggc ccgaacgccg accgccggcc | 3840 |
| ttcgatctcg ggtgtccccc tcgtcaattt cctctctcgg gtgcagccac gaaagtcgtg | 3900 |
| acgcaggtca cgaaatccgg ttacgaaaaa cgcaggtctt cgcaaaaacg tgagggtttc | 3960 |
| gcgtctcgcc ctagctattc gtatcgccgg gtcagaccca cgtgcagaaa agcccttgaa | 4020 |
| taacccggga ccgtggttac cgcgccgcct gcaccagggg gcttatataa gcccacacca | 4080 |
| cacctgtctc accacgcatt tctccaactc gcgacttttc ggaagaaatt gttatccacc | 4140 |
| tagtatagac tgccacctgc aggaccttgt gtcttgcagt ttgtattggt cccggccgtc | 4200 |
| gagctcgaca gatctgggct agggttggcc tggccgctcg gcactcccct ttagccgcgc | 4260 |
| gcatccgcgt tccagaggtg cgattcggtg tgtggagcat tgtcatgcgc ttgtgggggt | 4320 |
| cgttccgtgc gcggcgggtc cgccatgggc gccgacctgg gccctagggt ttgttttcgg | 4380 |
| gccaagcgag cccctctcac ctcgtcgccc ccccgcattc cctctctctt gcagcccata | 4440 |
| tggccatggc cgccgccgtg atcgtgcccc tgggcatcct gttcttcatc tccggcctgg | 4500 |
| tggtgaacct gctgcaggcc atctgctacg tgctgatccg ccccctgtcc aagaacacct | 4560 |
| accgcaagat caaccgcgtg gtggccgaga ccctgtggct ggagctggtg tggatcgtgg | 4620 |
| actggtgggc cggcgtgaag atccaggtgt tcgccgacaa cgagaccttc aaccgcatgg | 4680 |
| gcaaggagca cgccctggtg gtgtgcaacc accgctccga catcgactgg ctggtgggct | 4740 |
| ggatcctggc ccagcgctcc ggctgcctgg gctccgccct ggccgtgatg aagaagtcct | 4800 |
| ccaagttcct gcccgtgatc ggctggtcca tgtggttctc cgagtacctg ttcctggagc | 4860 |
| gcaactgggc caaggacgag tccaccctga agtccggcct gcagcgcctg aacgacttcc | 4920 |
| cccgcccctt ctggctggcc ctgttcgtgg agggcacccg cttcaccgag gccaagctga | 4980 |
| aggccgccca ggagtacgcc gcctcctccg agctgcccgt gccccgcaac gtgctgatcc | 5040 |
| cccgcaccaa gggcttcgtg tccgccgtgt ccaacatgcg ctccttcgtg cccgccatct | 5100 |
| acgacatgac cgtggccatc cccaagacct cccccccccc caccatgctg cgcctgttca | 5160 |
| agggccagcc ctccgtggtg cacgtgcaca tcaagtgcca ctccatgaag gacctgcccg | 5220 |
| agtccgacga cgccatcgcc cagtggtgcc gcgaccagtt cgtggccaag gacgccctgc | 5280 |
| tggacaagca catcgccgcc gacaccttcc ccggccagca ggagcagaac atcggccgcc | 5340 |
| ccatcaagtc cctggccgtg gtgctgtcct ggtcctgcct gctgatcctg ggcgccatga | 5400 |
| agttcctgca ctggtccaac ctgttctcct cctggaaggg catcgccttc tccgccctgg | 5460 |
| gcctgggcat catcaccctg tgcatgcaga tcctgatccg ctcctcccag tccgagcgct | 5520 |
| ccacccccgc caaggtggtg cccgccaagc ccaaggacaa ccacaacgac tccggctcct | 5580 |
| cctcccagac cgaggtggag aagcagaagt gaatgcatgc agcagcagct cggatagtat | 5640 |
| cgacacactc tggacgctgg tcgtgtgatg gactgttgcc gccacacttg ctgccttgac | 5700 |
| ctgtgaatat ccctgccgct tttatcaaac agcctcagtg tgtttgatct tgtgtgtacg | 5760 |
| cgcttttgcg agttgctagc tgcttgtgct atttgcgaat accaccccca gcatcccctt | 5820 |
| ccctcgtttc atatcgcttg catcccaacc gcaacttatc tacgctgtcc tgctatccct | 5880 |
| cagcgctgct cctgctcctg ctcactgccc ctcgcacagc cttggtttgg gctccgcctg | 5940 |
| tattctcctg gtactgcaac ctgtaaacca gcactgcaat gctgatgcac gggaagtagt | 6000 |
| gggaugggaa cacaaatgga cttaaggatc taagtaagat tcgaagcgct cgaccgtgcc | 6060 |
| ggacggactg cagccccatg tcgtagtgac cgccaatgta agtgggctgg cgtttccctg | 6120 |
| tacgtgagtc aacgtcactg cacgcgcacc accctctcga ccggcaggac caggcatcgc | 6180 |
| gagatacagc gcgagccaga cacggagtgc cgagctatgc gcacgctcca actagatatc | 6240 |
| atgtggatga tgagcatgaa ttcctttctt gcgctatgac acttccagca aaaggtaggg | 6300 |
| cgggctgcga gacggcttcc cggcgctgca tgcaacaccg atgatgcttc gaccccccga | 6360 |
| agctccttcg gggctgcatg ggcgctccga tgccgctcca gggcgagcgc tgtttaaata | 6420 |
| gccaggcccc cgattgcaaa gacattatag cgagctacca aagccatatt caaacaccta | 6480 |
| gatcactacc acttctacac aggccactcg agcttgtgat cgcactccgc taagggggcg | 6540 |
| cctcttcctc ttcgtttcag tcacaacccg caaacactag tatggctatc aagacgaaca | 6600 |
| ggcagcctgt ggagaagcct ccgttcacga tcgggacgct gcgcaaggcc atccccgcgc | 6660 |
| actgtttcga gcgctcggcg cttcgtagca gcatgtacct ggcctttgac atcgcggtca | 6720 |
| tgtccctgct ctacgtcgcg tcgacgtaca tcgaccctgc accggtgcct acgtgggtca | 6780 |
| agtacggcat catgtggccg ctctactggt tcttccaggt gtgtttgagg gttttggttg | 6840 |
| cccgtattga ggtcctggtg gcgcgcatgg aggagaaggc gcctgtcccg ctgacccccc | 6900 |
| cggctaccct cccggcacct tccagggcgc gtacgggaag aaccagtaga gcggccacat | 6960 |
| gatgccgtac ttgacccacg taggcaccgg tgcagggtcg atgtacgtcg acgcgacgta | 7020 |
| gagcagggac atgaccgcga tgtcaaaggc caggtacatg ctgctacgaa gcgccgagcg | 7080 |
| ctcgaaacag tgcgcgggga tggccttgcg cagcgtcccg atcgtgaacg gaggcttctc | 7140 |
| cacaggctgc ctgttcgtct tgatagccat ctcgaggcag cagcagctcg gatagtatcg | 7200 |
| acacactctg gacgctggtc gtgtgatgga ctgttgccgc cacacttgct gccttgacct | 7260 |
| gtgaatatcc ctgccgcttt tatcaaacag cctcagtgtg tttgatcttg tgtgtacgcg | 7320 |
| cttttgcgag ttgctagctg cttgtgctat ttgcgaatac cacccccagc atccccttcc | 7380 |
| ctcgtttcat atcgcttgca tcccaaccgc aacttatcta cgctgtcctg ctatccctca | 7440 |
| gcgctgctcc tgctcctgct cactgcccct cgcacagcct tggtttgggc tccgcctgta | 7500 |
| ttctcctggt actgcaacct gtaaaccagc actgcaatgc tgatgcacgg gaagtagtgg | 7560 |
| gatgggaaca caaatggaaa gctgtagagc tcttgttttc cagaaggagt tgctccttga | 7620 |
| gcctttcatt ctcagcctcg ataacctcca aagccgctct aattgtggag ggggttcgaa | 7680 |
| ccgaatgctg cgtgaacggg aaggaggagg agaaagagtg agcagggagg gattcagaaa | 7740 |
| tgagaaatga gaggtgaagg aacgcatccc tatgcccttg caatggacag tgtttctggc | 7800 |
| caccgccacc aagacttcgt gtcctctgat catcatgcga ttgattacgt tgaatgcgac | 7860 |
| ggccggtcag ccccggacct ccacgcaccg gtgctcctcc aggaagatgc gcttgtcctc | 7920 |
| cgccatcttg cagggctcaa gctgctccca aaactcttgg gcgggttccg gacggacggc | 7980 |
| taccgcgggt gcggccctga ccgccactgt tcggaagcag cggcgctgca tgggcagcgg | 8040 |
| ccgctgcggt gcgccacgga ccgcatgatc caccggaaaa gcgcacgcgc tggagcgcgc | 8100 |
| agaggaccac agagaagcgg aagagacgcc agtactggca agcaggctgg tcggtgccat | 8160 |
| ggcgcgctac taccctcgct atgactcggg tcctcggccg gctggcggtg ctgacaattc | 8220 |
| gtttagtgga gcagcgactc cattcagcta ccagtcgaac tcagtggcac agtgactccg | 8280 |
| ctcttc | 8286 |

Brassic napus LPAAT CDS
SEQ ID NO: 63
MAMAAAVIVPLGILFFISGLVVNLLQAVCYVLVRPMSKNTYRKINRVVAETLWLELVWIVDWWAGVKIQV
FADDETFNRMGKEHALVVCNHRSDIDWLVGWILAQRSGCLGSALAVMKKSSKFLPVIGWSMWFSEYLFLE
RNWAKDESTLQSGLQRLNDFPRPFWLALFVEGTRFTEAKLKAAQEYAASSELPVPRNVLIPRTKGFVSAV
SNMRSFVPAIYDMTVAIPKTSPPPTMLRLFKGQPSVVHVHIKCHSMKDLPEPEDEIAQWCRDQFVAKDAL
LDKHIAADTFPGQKEQNIGRPIKSLAVVVSWACLLTLGAMKFLHWSNLFSSWKGIALSAFGLGIITLCMQ
ILIRSSQSERSTPAKVAPAKPKDNHQSGPSSQTEVEEKQK
Mature native Protheca moriformis KASII amino acid sequence
SEQ ID NO: 64
AAAAADANPARPERRVVITGQGVVTSLGQTIEQFYSSLLEGVSGISQIQKFDTTGYTTTIAGEIKSLQ
LDPYVPKRWAKRVDDVIKYVYIAGKQALESAGLPIEAAGLAGAGLDPALCGVLIGTAMAGMTSFAAGV
EALTRGGVRKMNPFCIPFSISNMGGAMLAMDIGFMGPNYSISTACATGNYCILGAADHIRRGDANVML
AGGADAAIIPSGIGGFIACKALSKRNDEPERASRPWDADRDGFVMGEGAGVLVLEELEHAKRRGATIL
AELVGGAATSDAHHMTEPDPQGRGVRLCLERALERARLAPERVGYVNAHGTSTPAGDVAEYRAIRAVI
PQDSLRINSTKSMIGHLLGGAGAVEAVAAIQALRTGWLHPNLNLENPAPGVDPVVLVGPRKERAEDLD
VVLSNSFGFGGHNSCVIFRKYDE
Mature Prototheca moriformis Stearoyl Acyl-ACP desaturase (SAD2-1)
SEQ ID NO: 65
GAVAAPGRRAASRPLVVHAVASEAPLGVPPSVQRPSPVVYSKLDKQHRLTPERLELVQSMGQFAEERV
LPVLHPVDKLWQPQDFLPDPESPDFEDQVAELRARAKDLPDEYFVVLVGDMITEEALPTYMAMLNTLD
GVRDDTGAADHPWARWTRQWVAEENRHGDLLNKYCWLTGRVNMRAVEVTINNLIKSGMNPQTDNNPYL
GFVYTSFQERATKYSHGNTARLAAEHGDKGLSKICGLIASDEGRHEIAYTRIVDEFFRLDPEGAVAAY
ANMMRKQITMPAHLMDDMGHGEANPGRNLFADFSAVAEKIDVYDAEDYCRILEHLNARWKVDERQVSG
QAAADQEYVLGLPQRFRKLAEKTAAKRKRVARRPVAFSWISGREIMV
Nucleotide sequence of transforming DNA contained in pSZ3870
SEQ ID NO: 66
gctcttcacccaactcagataataccaatacccctccttctcctcctcatccattcagtacccccccccttctcttcccaaagcagcaagcgcgtg
gcttacagaagaacaatcggcttccgccaaagtcgccgagcactgcccgacggcggcgcgcccagcagcccgcttggccacacaggcaacga
atacattcaatagggggcctcgcagaatggaaggagcggtaaagggtacaggagcactgcgcacaaggggcctgtgcaggagtgactgact
gggcgggcagacggcgcaccgcgggcgcaggcaagcagggaagattgaagcggcagggaggaggatgctgattgaggggggcatcgcagt
ctctcttggacccgggataaggaagcaaatattcggccggttgggttgtgtgtgtgcacgttttcttcttcagagtcgtgggtgtgcttccaggga

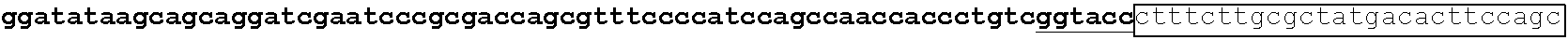

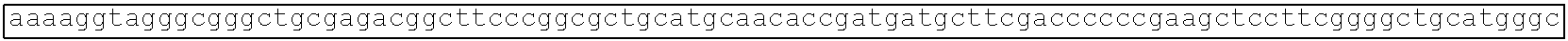

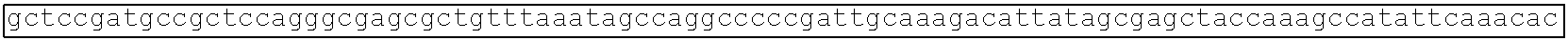

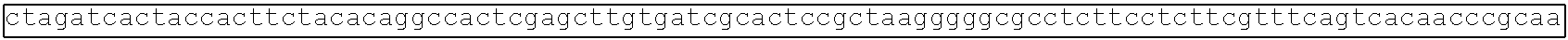

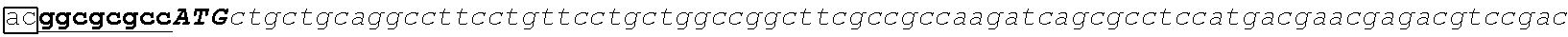

cgccccctggtgcacttcacccccaacaagggctggatgaacgaccccaacggcctgtggtacgacgagaaggacgccaagtggcacct
gtacttccagtacaacccgaacgacaccgtctgggggacgcccttgttctggggccacgccacgtccgacgacctgaccaactgggagga
ccagcccatcgccatcgccccgaagcgcaacgactccggcgccttctccggctccatggtggtggactacaacaacacctccggcttcttca
acgacaccatcgacccgcgccagcgctgcgtggccatctggacctacaacaccccggagtccgaggagcagtacatctcctacagcctgg
acggcggctacaccttcaccgagtaccagaagaaccccgtgctggccgccaactccacccagttccgcgacccgaaggtcttctggtacga
gccctcccagaagtggatcatgaccgcggccaagtcccaggactacaagatcgagatctactcctccgacgacctgaagtcctggaagct
ggagtccgcgttcgccaacgagggcttcctcggctaccagtacgagtgccccggcctgatcgaggtccccaccgagcaggaccccagcaa
gtcctactgggtgatgttcatctccatcaaccccggcgccccggccggcggctccttcaaccagtacttcgtcggcagcttcaacggcaccca
cttcgaggccttcgacaaccagtcccgcgtggtggacttcggcaaggactactacgccctgcagaccttcttcaacaccgacccgacctacg
ggagcgccctgggcatcgcgtgggcctccaactgggagtactccgccttcgtgcccaccaacccctggcgctcctccatgtccctcgtgcgca
agttctccctcaacaccgagtaccaggccaacccggagacggagctgatcaacctgaaggccgagccgatcctgaacatcagcaacgcc
ggcccctggagccggttcgccaccaacaccacgttgacgaaggccaacagctacaacgtcgacctgtccaacagcaccggcaccctgga
gttcgagctggtgtacgccgtcaacaccacccagacgatctccaagtccgtgttcgcggacctctccctctggttcaagggcctggaggacc
ccgaggagtacctccgcatgggcttcgaggtgtccgcgtcctccttcttcctggaccgcgggaacagcaaggtgaagttcgtgaaggaga
acccctacttcaccaaccgcatgagcgtgaacaaccagcccttcaagagcgagaacgacctgtcctactacaaggtgtacggcttgctgga
ccagaacatcctggagctgtacttcaacgacggcgacgtcgtgtccaccaacacctacttcatgaccaccgggaacgccctgggctccgtg

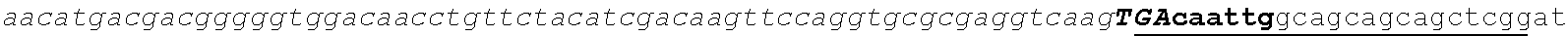

agtatcgacacactctggacgctggtcgtgtgatggactgttgccgccacacttgctgccttgacctgtgaatatccctgccgcttttatcaaacag
cctcagtgtgtttgatcttgtgtgtacgcgcttttgcgagttgctagctgcttgtgctatttgcgaataccacccccagcatccccttccctcgtttcat
atcgcttgcatcccaaccgcaacttatctacgctgtcctgctatccctcagcgctgctcctgctcctgctcactgcccctcgcacagccttggtttgg
gctccgcctgtattctcctggtactgcaacctgtaaaccagcactgcaatgctgatgcacgggaagtagtgggatgggaacacaaatggaggat
cccgcgtctcgaacagagcgcgcagaggaacgctgaaggtctcgcctctgtcgcacctcagcgcggcatacaccacaataaccacctgacgaa
tgcgcttggttcttcgtccattagcgaagcgtccggttcacacacgtgccacgttggcgaggtggcaggtgacaatgatcggtggagctgatggtc

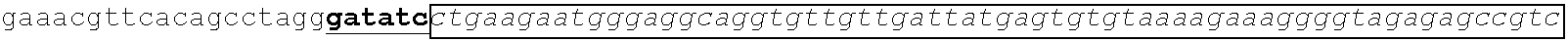

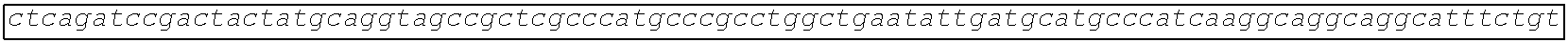

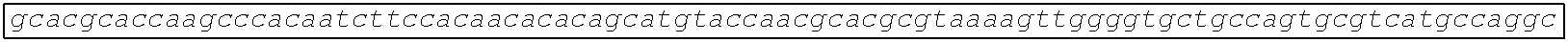

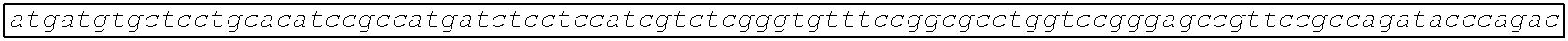

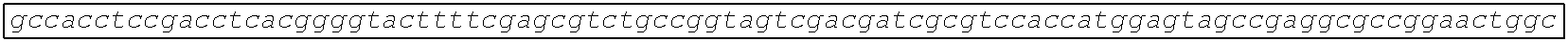

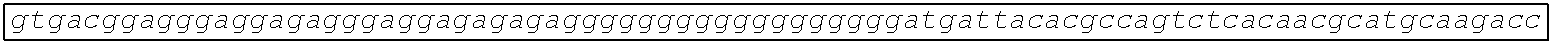

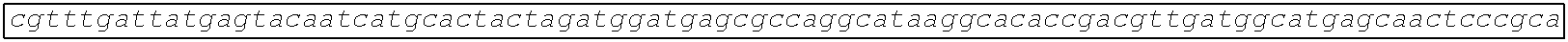

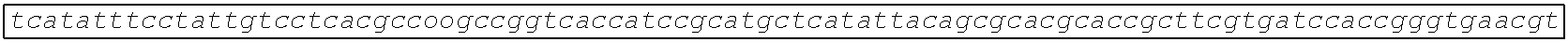

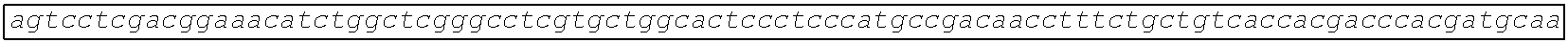

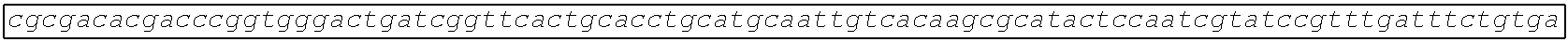

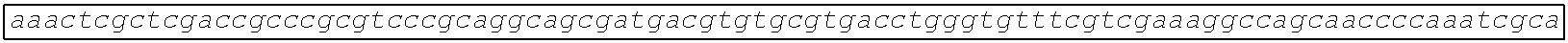

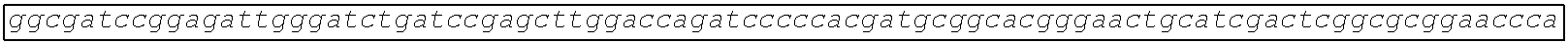

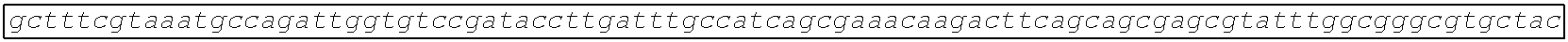

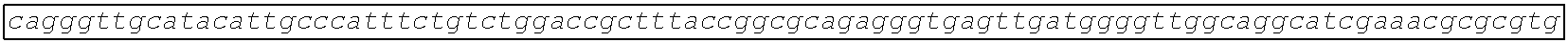

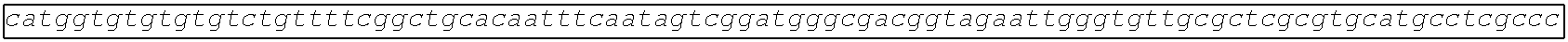

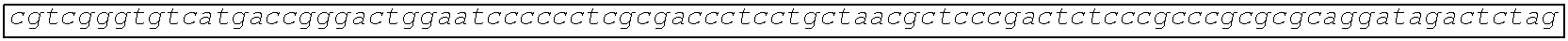

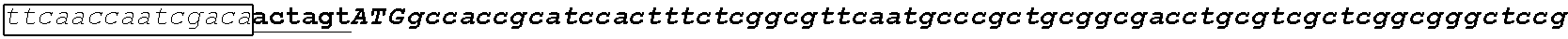

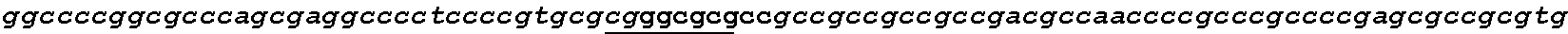

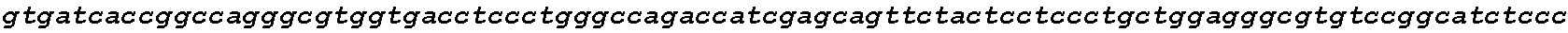

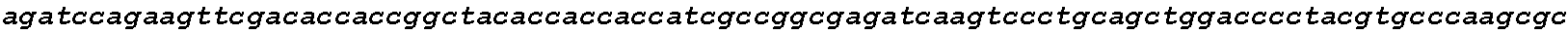

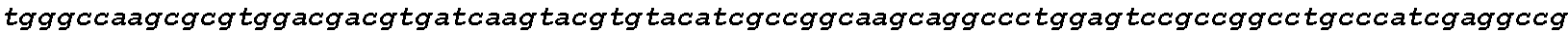

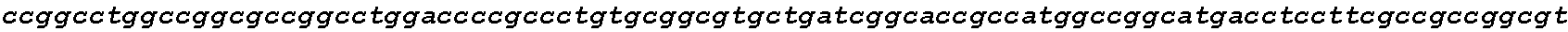

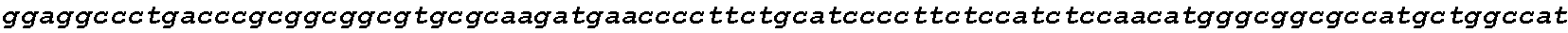

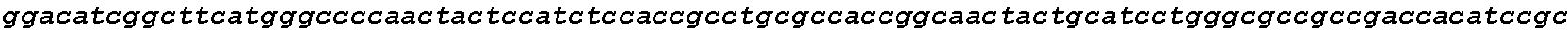

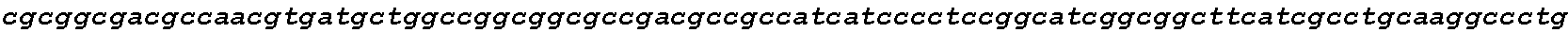

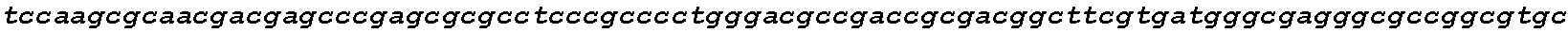

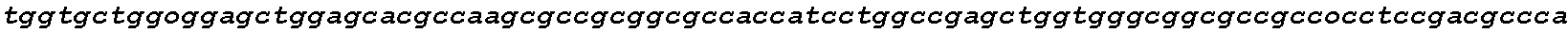

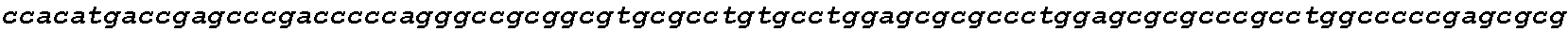

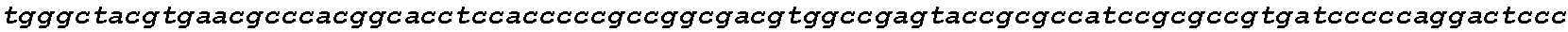

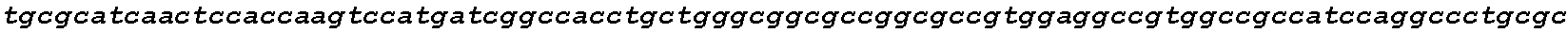

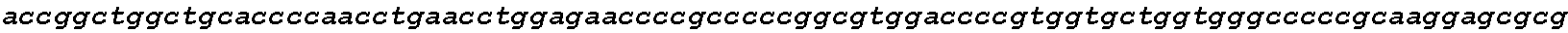

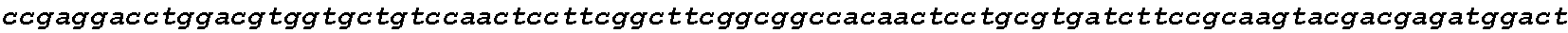

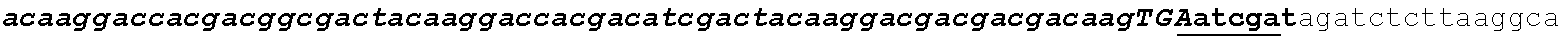

gcagcagctcggatagtatcgacacactctggacgctggtcgtgtgatggactgttgccgccacacttgctgccttgacctgtgaatatccctgcc
gcttttatcaaacagcctcagtRtRtttgatcttgtgtgtacgcgcttttgcgagttgctagctgcttgtgctatttgcgaataccacccccagcatcc
ccttccctcgtttcatatcgcttgcatcccaaccgcaacttatctacgctgtcctgctatccctcagcgctgctcctgctcctgctcactgcccctcgc
acagccttggtttgggrtccgcctgtattctcctggtactgcaacctgtaaaccagcactgcaatgctgatgcacgggaagtagtgggatgggaa
cacaaatggaaagcttaattaagagctcttgttttccagaaggagttgctccttgagcctttcattctcagcctcgataacctccaaagccgctct
aattgtggagggggttcgaaccgaatgctgcgtgaacgggaaggaggaggagaaagagtgagcagggagggattcagaaatgagaaatg
agaggtgaaggaacgcatccctatgcccttgcaatggacagtgtttctggccaccgccaccaagacttcgtgtcctctgatcatcatgcgattga
ttacgttgaatgcgacggccggtcagccccggacctccacgcaccggtgctcctccaggaagatgcgcttgtcctccgccatcttgcagggctca
agctgctcccaaaactcttgggcgggttccggacggacggctaccgcgggtgcggccctgaccgccactgttcggaagcagcggcgctgcatg
ggcagcggccgctgcggtgcgccacggaccgcatgatccaccggaaaagcgcacgcgctggagcgcgcagaggaccacagagaagcggaa
gagacgccagtactggcaagcaggctggtcggtgccatggcgcgctactaccctcgctatgactcgggtcctcggccggctggcggtgctgaca
attcgtttagtggagcagcgactccattcagctaccagtcgaactcagtggcacagtgactccgctcttc
Nucleotide sequence of PmUAPA1 promoter contained in pSZ2533
SEQ ID NO: 67

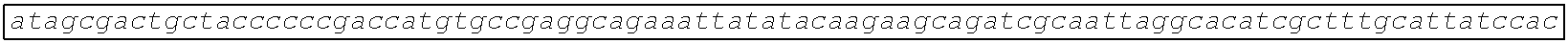

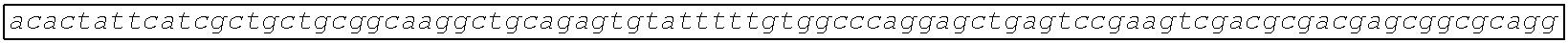

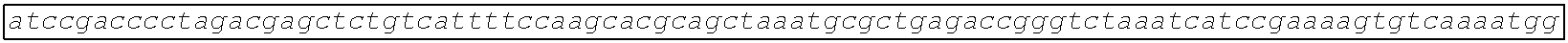

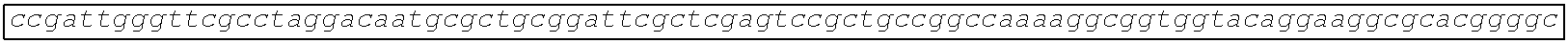

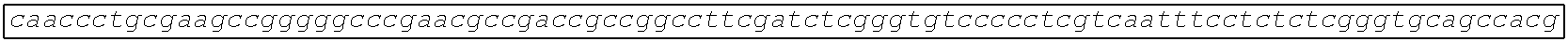

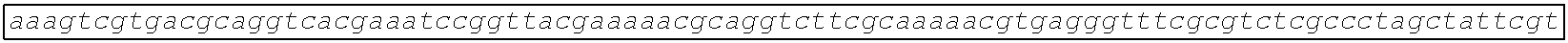

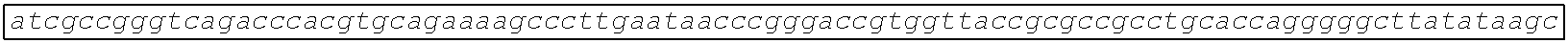

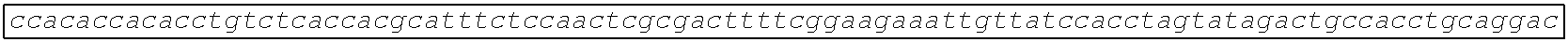

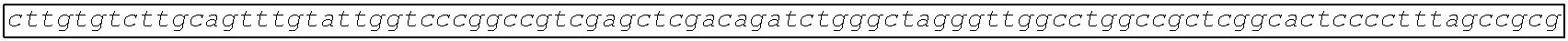

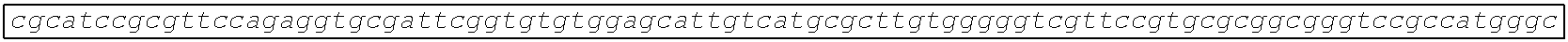

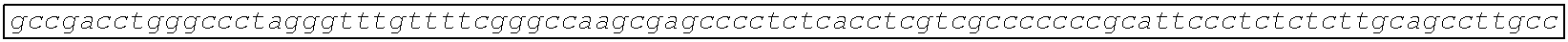

Nucleotide sequence of PmHXT1 promoter contained in pSZ3869
SEQ ID NO: 68

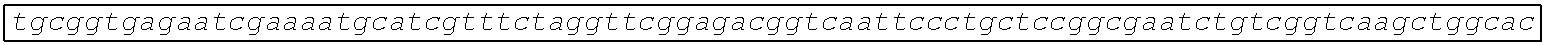

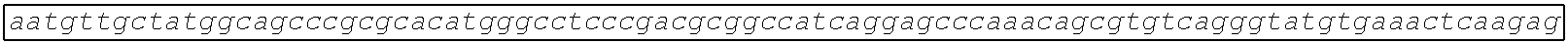

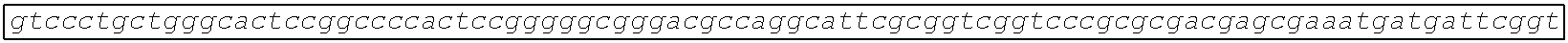

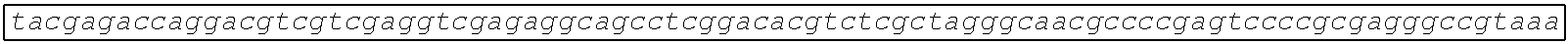

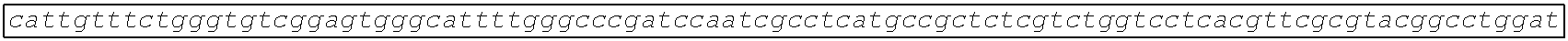

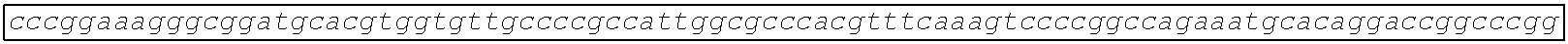

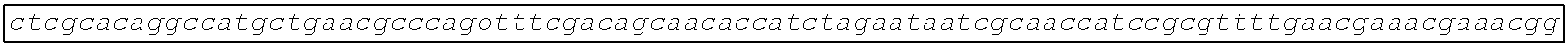

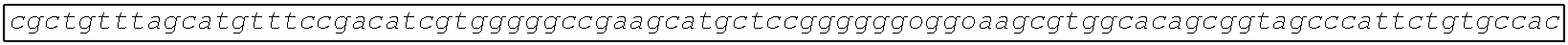

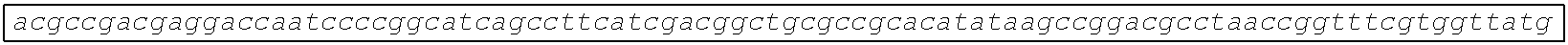

Nucleotide sequence of PmSOD promoter contained in pSZ3935
SEQ ID NO: 69

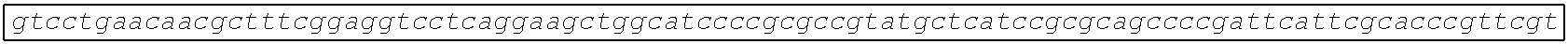

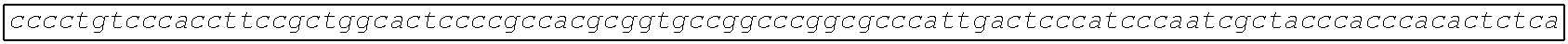

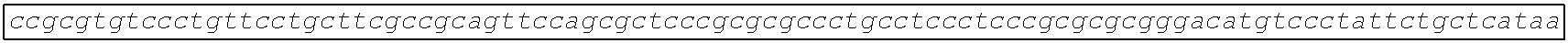

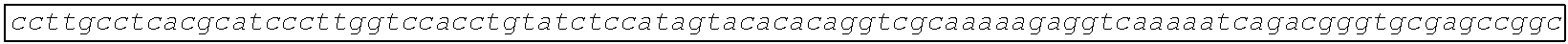

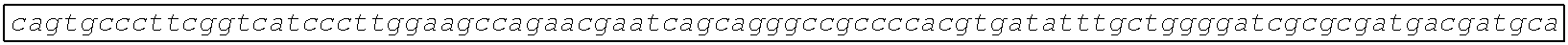

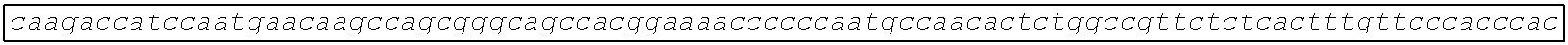

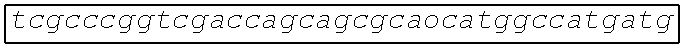

Nucleotide sequence of PmATPB1 promoter contained in pSZ3936
SEQ ID NO: 70

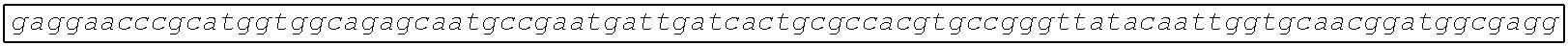

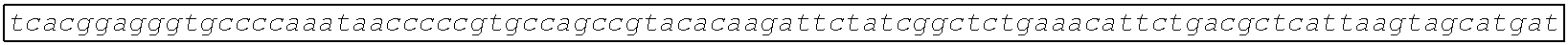

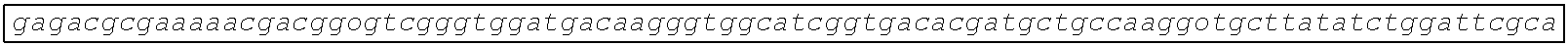

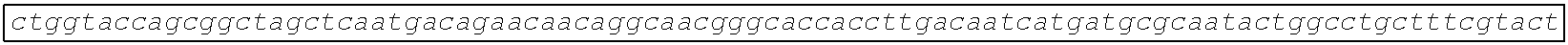

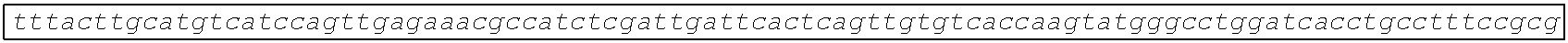

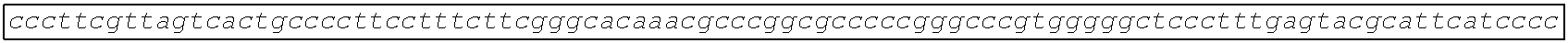

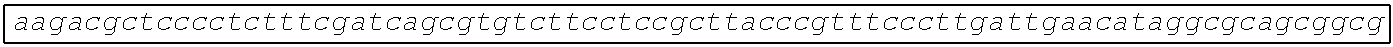

Nucleotide sequence of PmEf1-1 promoter contained in pSZ3937
SEQ ID NO: 71

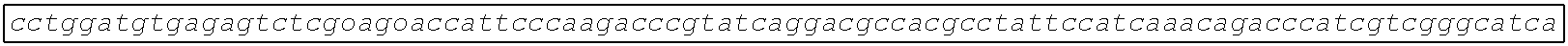

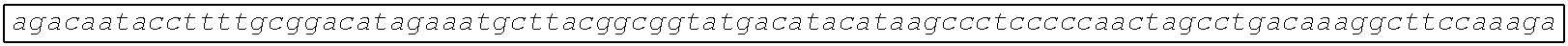

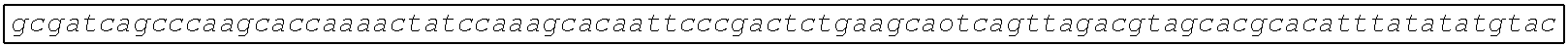

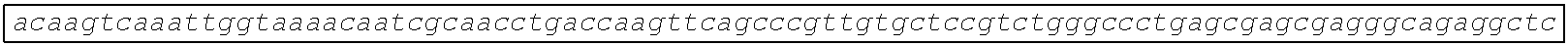

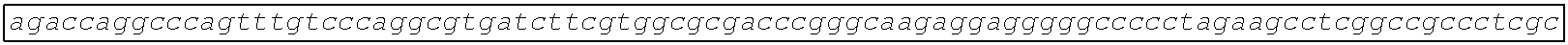

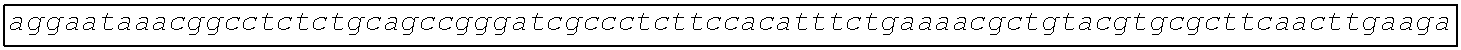

Nucleotide sequence of PmEf1-2 promoter contained in pSZ3938
SEQ ID NO: 72

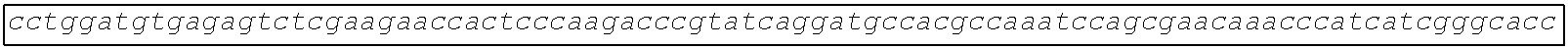

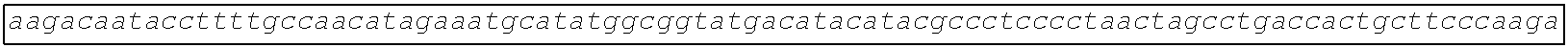

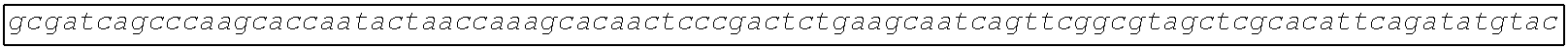

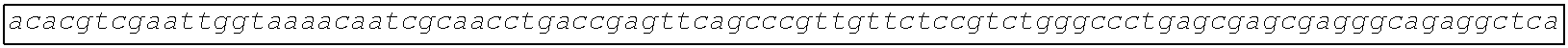

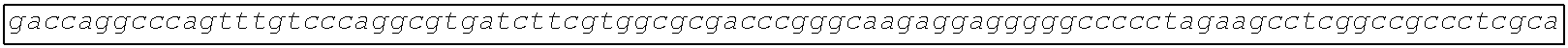

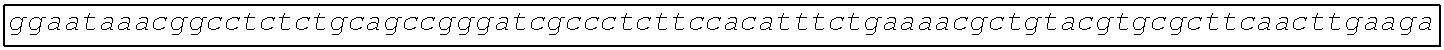

Nucleotide sequence of PmACP1 promoter contained in pSZ3939
SEQ ID NO: 73

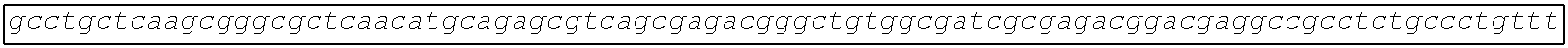

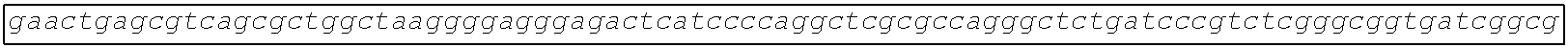

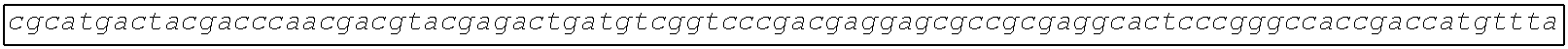

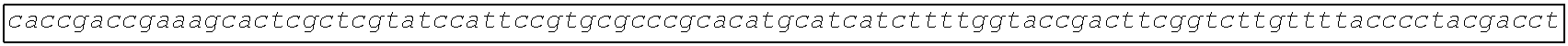

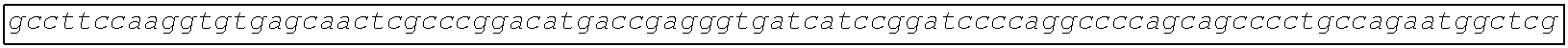

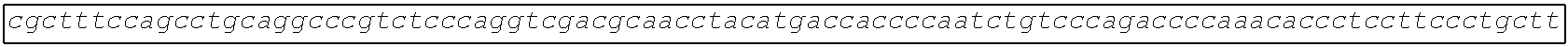

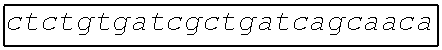

Nucleotide sequence of PmACP2 promoter contained in pSZ3940
SEQ ID NO: 74

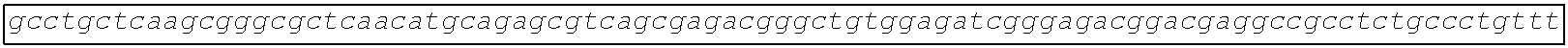

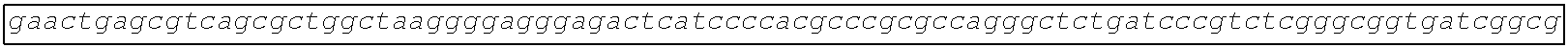

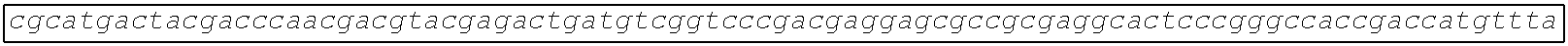

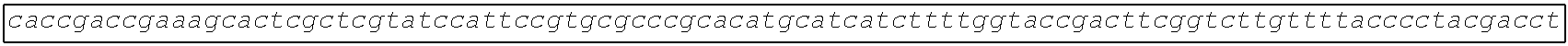

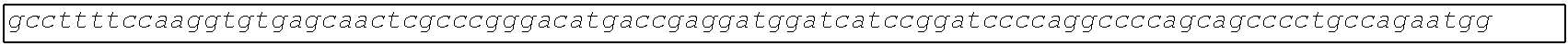

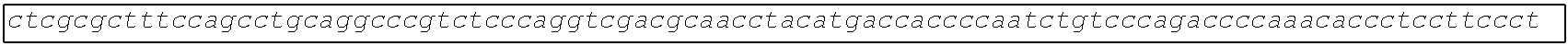

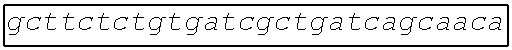

Nucleotide sequence of PmC1LYR1 promoter contained in pSZ3941
SEQ ID NO: 75

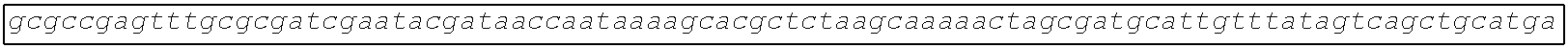

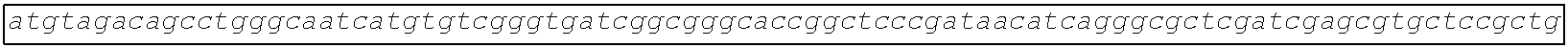

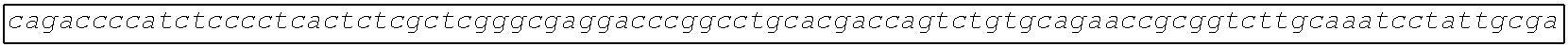

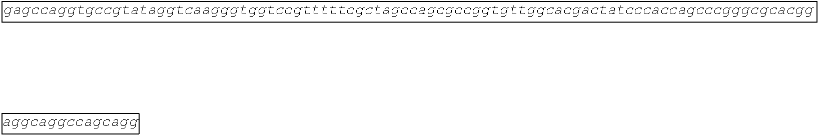

Nucleotide sequence of PmAMT1-1 promoter contained in pSZ3942
SEQ ID NO: 76

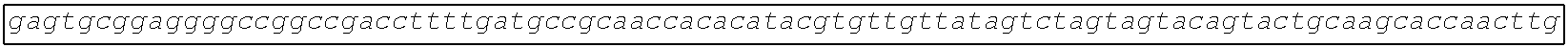

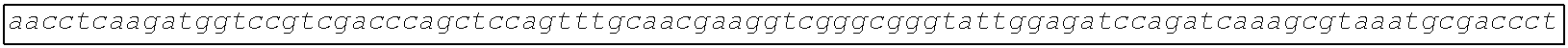

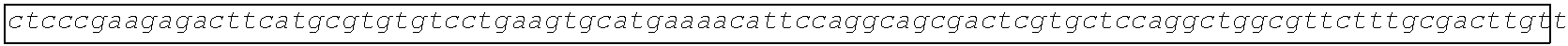

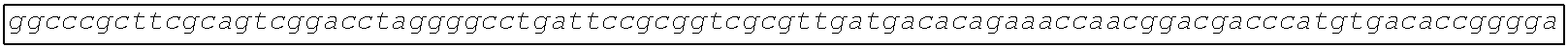

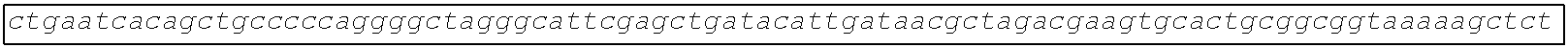

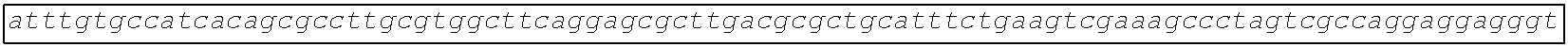

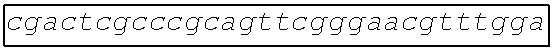

Nucleotide sequence of PmAMT1-2 promoter contained in pSZ3943
SEQ ID NO: 77

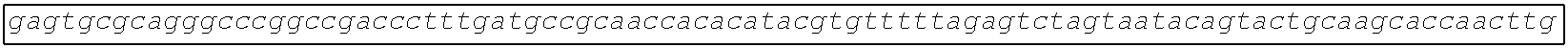

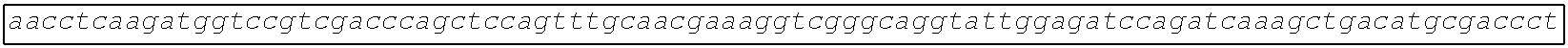

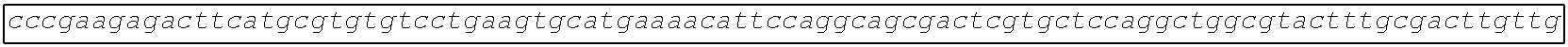

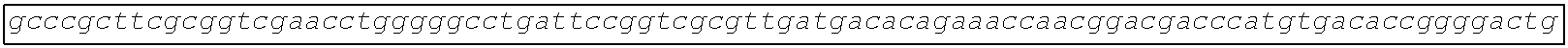

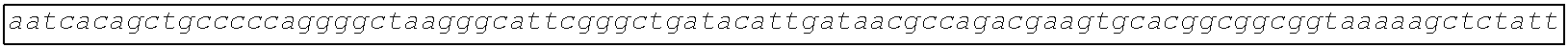

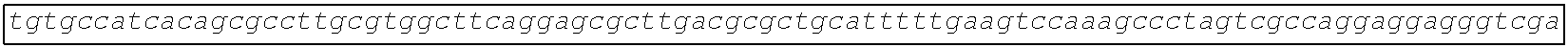

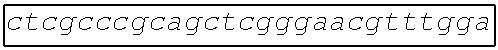

Nucleotide sequence of PmAMT3-1 promoter contained in pSZ3944
SEQ ID NO: 78

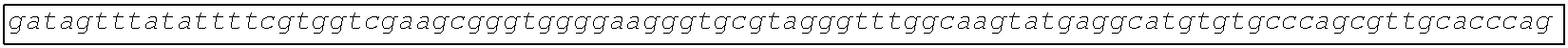

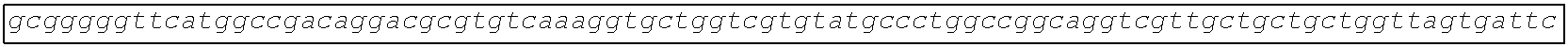

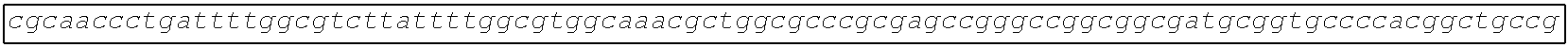

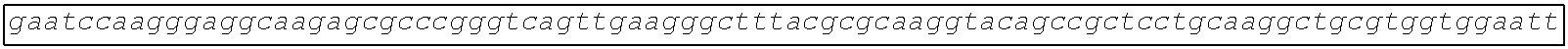

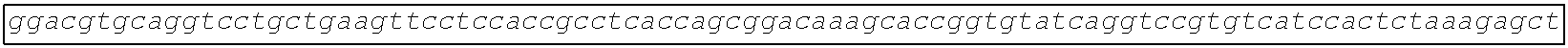

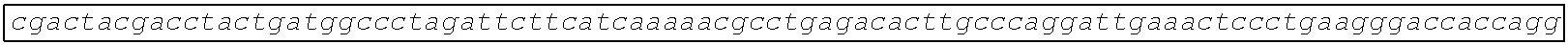

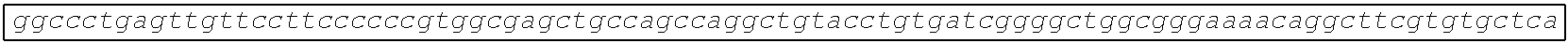

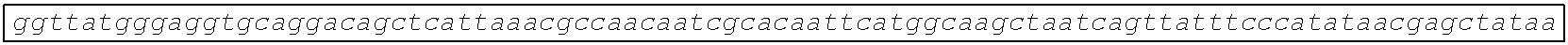

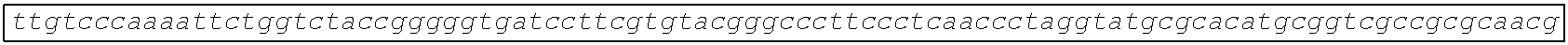

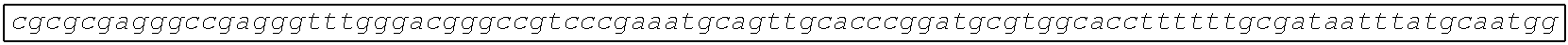

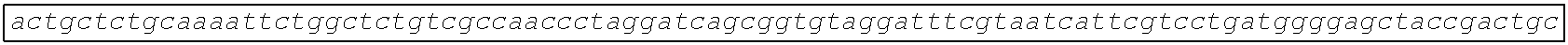

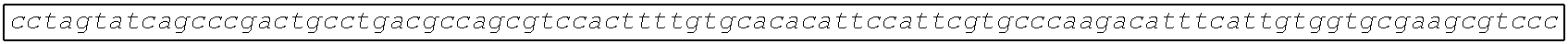

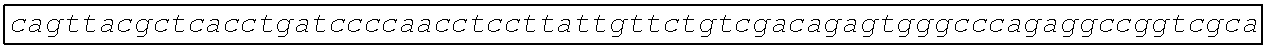

Nucleotide sequence of PmAMT3-2 promoter contained in pSZ3945
SEQ ID NO: 79

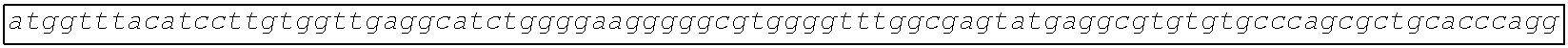

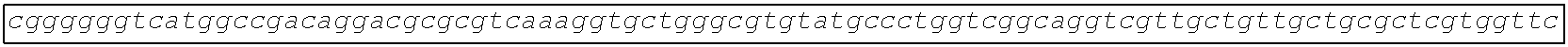

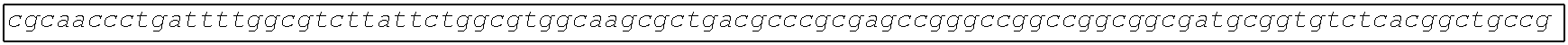

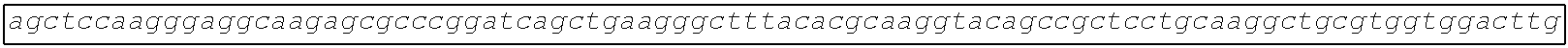

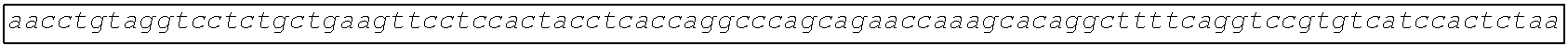

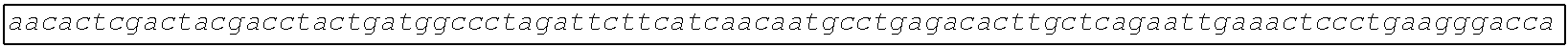

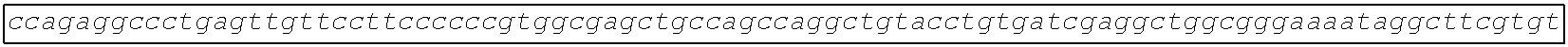

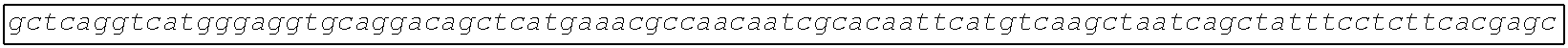

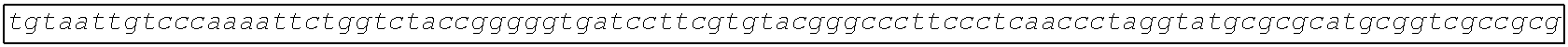

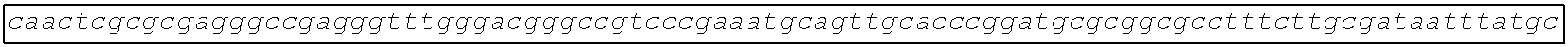

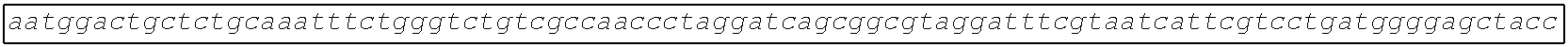

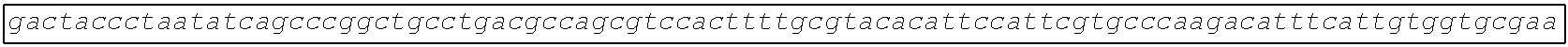

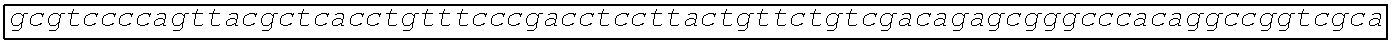

Nucleotide sequence of transforming DNA contained in pSZ4768 (D3870)
SEQ ID NO: 80
gctcttcgcgaaggtcattttccagaacaacgaccatggcttgtcttagcgatcgctcgaatgactgctagtgagtcgtacgctcgacccagtcg
ctcgcaggagaacgcggcaactgccgagcttcggcttgccagtcgtgactcgtatgtgatcaggaatcattggcattggtagcattataattcg
gcttccgcgctgtttatgggcatggcaatgtctcatgcagtcgaccttagtcaaccaattctgggtggccagctccgggcgaccgggctccgtgt
cgccgggcaccacctcctgccatgagtaacagggccgccctctcctcccgacgttggcccactgaataccgtgtcttggggccctacatgatggg
ctgcctagtcgggcgggacgcgcaactgcccgcgcaatctgggacgtggtctgaatcctccaggcgggtttccccgagaaagaaagggtgccg
atttcaaagcagagccatgtgccgggccctgtggcctgtgttggcgcctatgtagtcaccccccctcacccaattgtcgccagtttgcgcaatcc

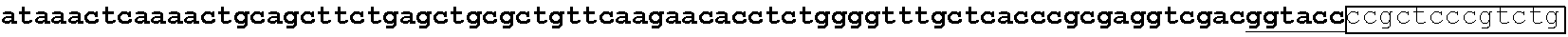

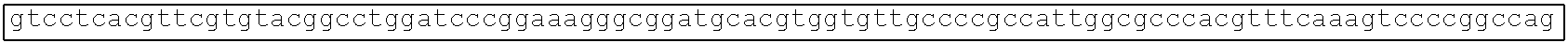

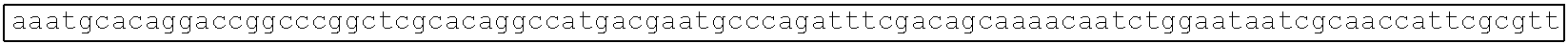

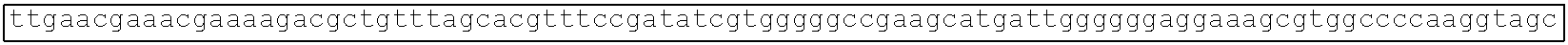

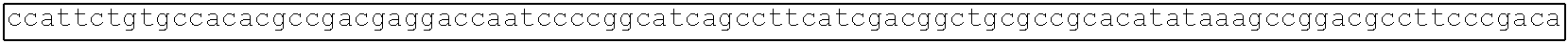

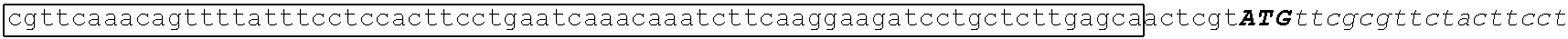

gacggcctgcatctccctgaagggcgtgttcggcgtctccccctcctacaacggcctgggcctgacgccccagatgggctgggacaactgg
aacacgttcgcctgcgacgtctccgagcagctgctgctggacacggccgaccgcatctccgacctgggcctgaaggacatgggctacaag
tacatcatcctggacgactgctggtcctccggccgcgactccgacggcttcctggtcgccgacgagcagaagttccccaacggcatgggcc
acgtcgccgaccacctgcacaacaactccttcctgttcggcatgtactcctccgcgggcgagtacacgtgcgccggctaccccggctccctgg
gccgcgaggaggaggacgcccagttcttcgcgaacaaccgcgtggactacctgaagtacgacaactgctacaacaagggccagttcggc
acgcccgagatctcctaccaccgctacaaggccatgtccgacgccctgaacaagacgggccgccccatcttctactccctgtgcaactgggg
ccaggacctgaccttctactggggctccggcatcgcgaactcctggcgcatgtccggcgacgtcacggcggagttcacgcgccccgactccc
gctgcccctgcgacggcgacgagtacgactgcaagtacgccggcttccactgctccatcatgaacatcctgaacaaggccgcccccatggg
ccagaacgcgggcgtcggcggctggaacgacctggacaacctggaggtcggcgtcggcaacctgacggacgacgaggagaaggcgca
cttctccatgtgggccatggtgaagtcccccctgatcatcggcgcgaacgtgaacaacctgaaggcctcctcctactccatctactcccaggc
gtccgtcatcgccatcaaccaggactccaacggcatccccgccacgcgcgtctggcgctactacgtgtccgacacggacgagtacggccag
ggcgagatccagatgtggtccggccccctggacaacggcgaccaggtcgtggcgctgctgaacggcggctccgtgtcccgccccatgaac
acgaccctggaggagatcttcttcgactccaacctgggctccaagaagctgacctccacctgggacatctacgacctgtgggcgaaccgcg
tcgacaactccacggcgtccgccatcctgggccgcaacaagaccgccaccggcatcctgtacaacgccaccgagcagtcctacaaggacg
gcctgtccaagaacgacacccgcctgttcggccagaagatcggctccctgtcccccaacgcgatcctgaacacgaccgtccccgcccacgg

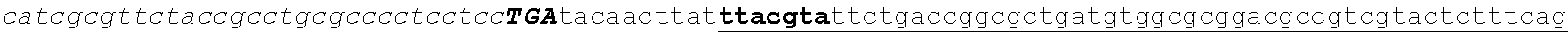

actttactcttgaggaattgaacctttctcgcttgctggcatgtaaacattggcgcaattaattgtgtgatgaagaaagggtggcacaagatggat
cgcgaatgtacgagatcgacaacgatggtgattgttatgaggggccaaacctggctcaatcttgtcgcatgtccggcgcaatgtgatccagcggc
gtgactctcgcaacctggtagtgtgtgcgcaccgggtcgctttgattaaaactgatcgcattgccatcccgtcaactcacaagcctactctagctcc
cattgcgcactcgggcgcccggctcgatcaatgttctgagcggagggcgaagcgtcaggaaatcgtctcggcagctggaagcgcatggaatgcg
gagcggagatcgaatcaggatcccgcgtctcgaacagagcgcgcagaggaacgctgaaggtctcgcctctgtcgcacctcagcgcggcataca
ccacaataaccacctgacgaatgcgcttggttcttcgtccattagcgaagcgtccggttcacacacgtgccacgttggcgaggtggcaggtgaca

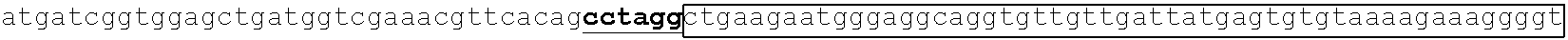

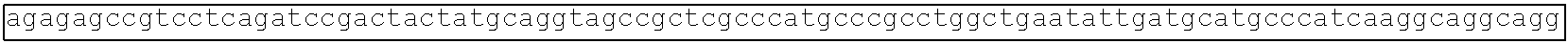

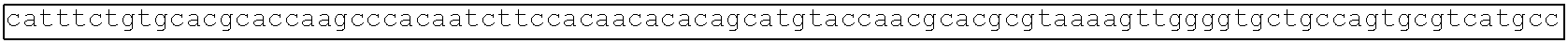

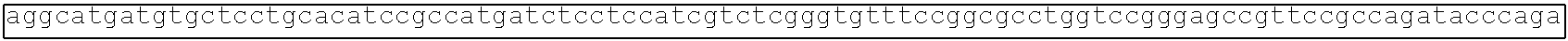

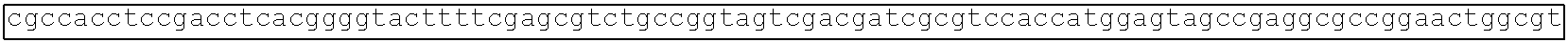

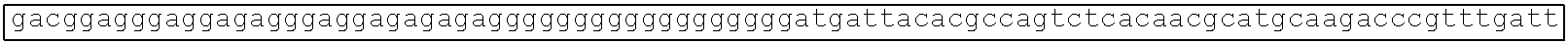

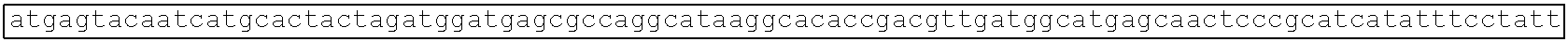

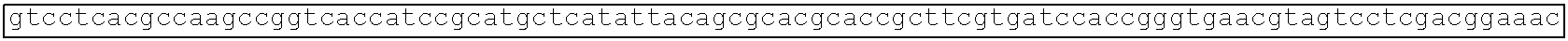

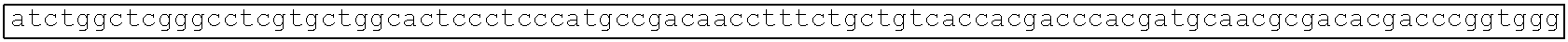

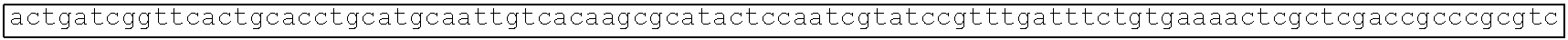

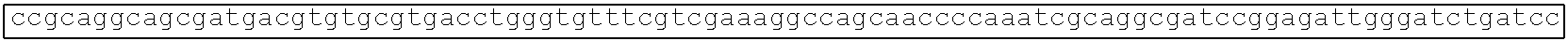

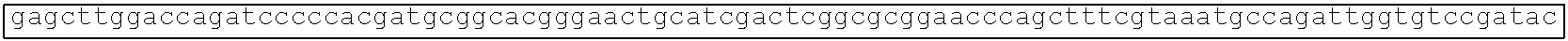

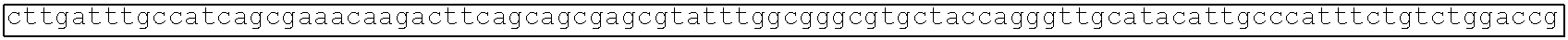

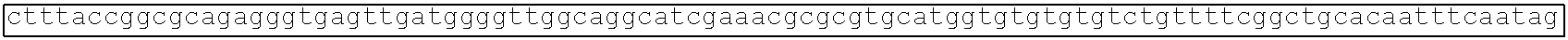

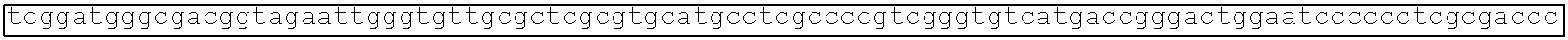

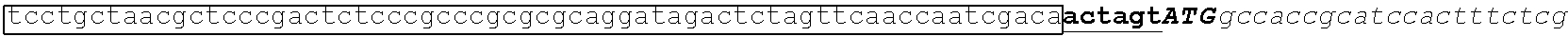

gcgttcaatgcccgctgcggcgacctgcgtcgctcggcgggctccgggccccggcgcccagcgaggcccctccccgtgcgcgggcgcgccg
ccgccgccgccgacgccaaccccgcccgccccgagcgccgcgtggtgatcaccggccagggcgtggtgacctccctgggccagaccatcg
agcagttctactcctccctgctggagggcgtgtccggcatctcccagatccagaagttcgacaccaccggctacaccaccaccatcgccggc
gagatcaagtccctgcagctggacccctacgtgcccaagcgctgggccaagcgcgtggacgacgtgatcaagtacgtgtacatcgccggc
aagcaggccctggagtccgccggcctgcccatcgaggccgccggcctggccggcgccggcctggaccccgccctgtgcggcgtgctgatc
ggcaccgccatggccggcatgacctccttcgccgccggcgtggaggccctgacccgcggcggcgtgcgcaagatgaaccccttctgcatcc
ccttctccatctccaacatgggcggcgccatgctggccatggacatcggcttcatgggccccaactactccatctccaccgcctgcgccaccg
gcaactactgcatcctgggcgccgccgaccacatccgccgcggcgacgccaacgtgatgctggccggcggcgccgacgccgccatcatcc
cctccggcatcggcggcttcatcgcctgcaaggccctgtccaagcgcaacgacgagcccgagcgcgcctcccgcccctgggacgccgaccg
cgacggcttcgtgatgggcgagggcgccggcgtgctggtgctggaggagctggagcacgccaagcgccgcggcgccaccatcctggccg
agctggtgggcggcgccgccacctccgacgcccaccacatgaccgagcccgacccccagggccgcggcgtgcgcctgtgcctggagcgcg
ccctggagcgcgcccgcctggcccccgagcgcgtgggctacgtgaacgcccacggcacctccacccccgccggcgacgtggccgagtacc
gcgccatccgcgccgtgatcccccaggactccctgcgcatcaactccaccaagtccatgatcggccacctgctgggcggcgccggcgccgt
ggaggccgtggccgccatccaggccctgcgcaccggctggctgcaccccaacctgaacctggagaaccccgcccccggcgtggaccccgt
ggtgctggtgggcccccgcaaggagcgcgccgaggacctggacgtggtgctgtccaactccttcggcttcggcggccacaactcctgcgtg
atcttccgcaagtacgacgagatggactacaaggaccacgacggcgactacaaggaccacgacatcgactacaaggacgacgacgac

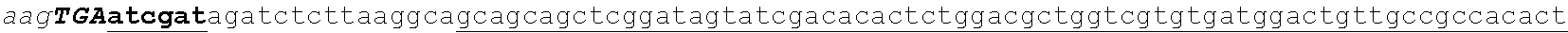

tgctgccttgacctgtgaatatccctgccgcttttatcaaacagcctcagtgtgtttgatcttgtgtgtacgcgcttttgcgagttgctagctgcttgtg
ctatttgcgaataccacccccagcatccccttccctcgtttcatatcgcttgcatcccaaccgcaacttatctacgctgtcctgctatccctcagcgct
gctcctgctcctgctcactgcccctcgcacagccttggtttgggctccgcctgtattctcctggtactgcaacctgtaaaccagcactgcaatgctga

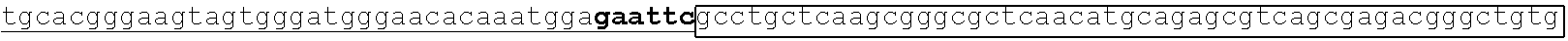

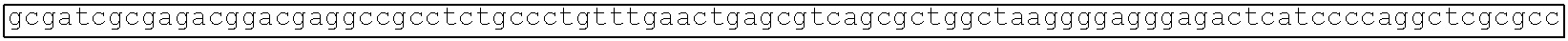

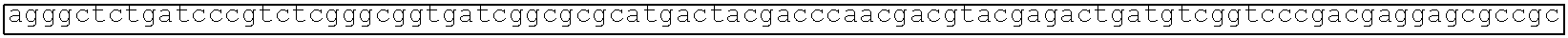

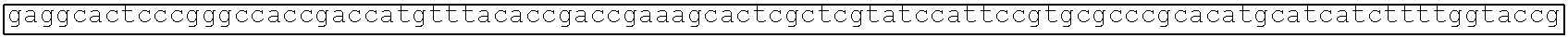

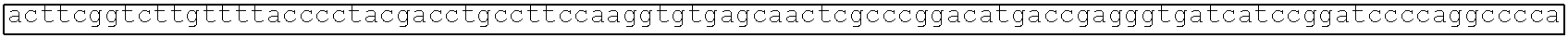

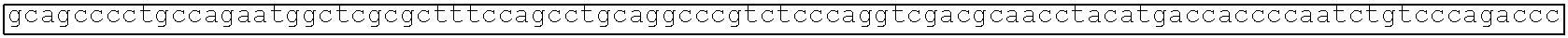

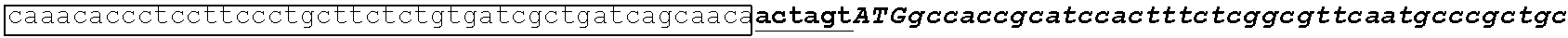

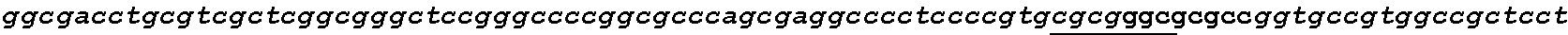

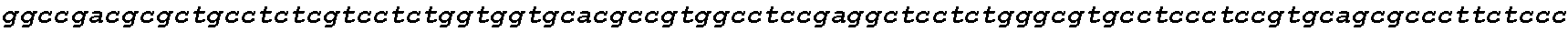

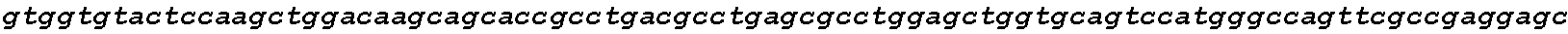

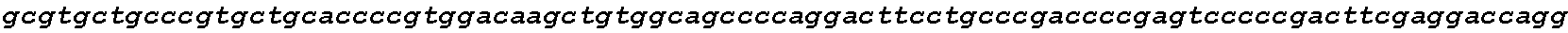

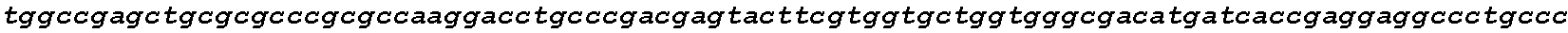

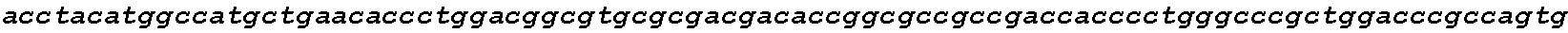

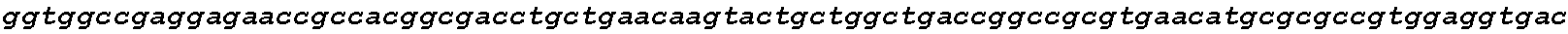

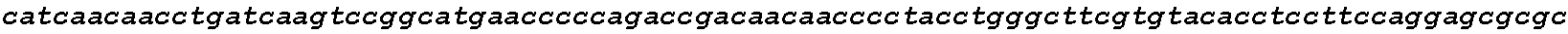

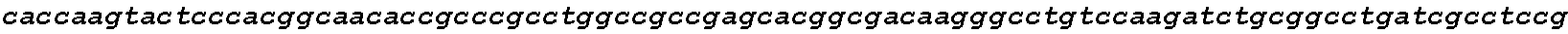

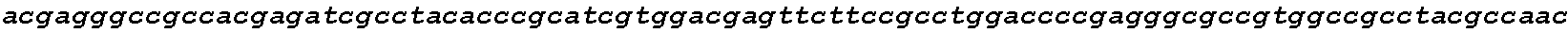

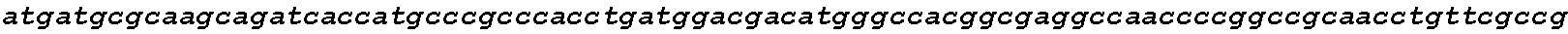

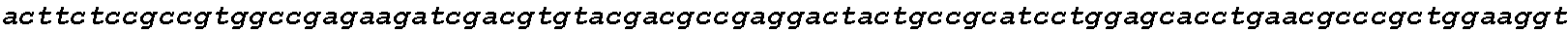

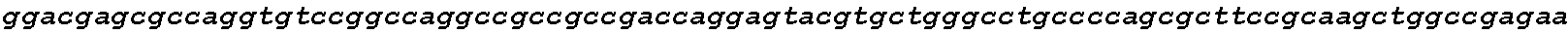

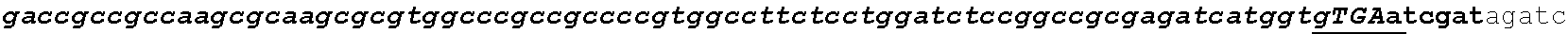

tcttaaggcagcagcagctcggatagtatcgacacactctggacgctggtcgtgtgatggactgttgccgccacacttgctgccttgacctgtgaa
tatccctgccgcttttatcaaacagcctcagtgtgtttgatcttgtgtgtacgcgcttttgcgagttgctagctgcttgtgctatttgcgaataccacc
cccagcatccccttccctcgtttcatatcgcttgcatcccaaccgcaacttatctacgctgtcctgctatccctcagcgctgctcctgctcctgctcac
tgcccctcgcacagccttggtttgggctccgcctgtattctcctggtactgcaacctgtaaaccagcactgcaatgctgatgcacgggaagtagtg
ggatgggaacacaaatggaaagcttaattaagagctcctcactcagcgcgcctgcgcggggatgcggaacgccgccgccgccttgtcttttgca
cgcgcgactccgtcgcttcgcgggtggcacccccattgaaaaaaacctcaattctgtttgtggaagacacggtgtacccccaaccacccacctg
cacctctattattggtattattgacgcgggagcgggcgttgtactctacaacgtagcgtctctggttttcagctggctcccaccattgtaaattctt
gctaaaatagtgcgtggttatgtgagaggtatggtgtaacagggcgtcagtcatgttggttttcgtgctgatctcgggcacaaggcgtcgtcga
cgtgacgtgcccgtgatgagagcaataccgcgctcaaagccgacgcatggcctttactccgcactccaaacgactgtcgctcgtatttttcggat
atctattttttaagagcgagcacagcgccgggcatgggcctgaaaggcctcgcggccgtgctcgtggtgggggccgcgagcgcgtggggcatc
gcggcagtgcaccaggcgcagacggaggaacgcatggtgagtgcgcatcacaagatgcatgtcttgttgtctgtactataatgctagagcatc
accaggggcttagtcatcgcacctgctttggtcattacagaaattgcacaagggcgtcctccgggatgaggagatgtaccagctcaagctgga
gcggcttcgagccaagcaggagcgcggcgcatgacgacctacccacatgcgaagagc
Prothcca moriformis SAD2-2v3 promoter
SEQ ID NO: 81
GTGAAAACTCGCTCGACCGCCCGCGTCCCGCAGGCAGCGATGACGTGTGCGTGACCTGGGTGTTTCGT
CGAAAGGCCAGCAACCCCAAATCGCAGGCGATCCGGAGATTGGGATCTGATCCGAGCTTGGACCAGAT
CCCCCACGATGCGGCACGGGAACTGCATCGACTCGGCGCGGAACCCAGCTTTCGTAAATGCCAGATTG
GTGTCCGATACCTTGATTTGCCATCAGCGAAACAAGACTTCAGCAGCGAGCGTATTTGGCGGGCGTGC
TACCAGGGTTGCATACATTGCCCATTTCTGTCTGGACCGCTTTACCGGCGCAGAGGGTGAGTTGATGG
GGTTGGCAGGCATCGAAACGCGCGTGCATGGTGTGTGTGTCTGTTTTCGGCTGCACAATTTCAATAGT
CGGATGGGCGACGGTAGAATTGGGTGTTGCGCTCGCGTGCATGCCTCGCCCCGTCGGGTGTCATGACC
GGGACTGGAATCCCCCCTCGCGACCCTCCTGCTAACGCTCCCGACTCTCCCGCCCGCGCGCAGGATAG
ACTCTAGTTCAACCAATCGACA
Limnanthes douglasii (LimdLPAAT, Uniprot Accession No: Q42870)
SEQ ID NO: 82
MAKTRTSSLRNRRQLKPAVAATADDDKDGVFMVLLSCFKIFVCFAIVLITAVAWGLIMVL
LLPWPYMRIRLGNLYGHIIGGLVIWIYGIPIKIQGSEHTKKRAIYISNHASPIDAFFVMW
LAPIGTVGVAKKEVIWYPLLGQLYTLAHHIRIDRSNPAAAIQSMKEAVRVITEKNLSLIM
FPEGTRSRDGRLLPFKKGFVHLALQSHLPIVPMILTGTHLAWRKGTFRVRPVPITVKYLP
PINTDDWTVDKIDDYVKMIHDVYVRNLPASQKPLGSTNRSN
Limnanthes alba (LimaLPAAT, Unirprot Accession No: Q42868)
SEQ ID NO: 83
MAKTRTSSLRNRRQLKTAVAATADDDKDGIFMVLLSCFKIFVCFAIVLITAVAWGLIMVL
LLPWPYMRIRLGNLYGHIIGGLVIWLYGIPIEIQGSEHTKKRAIYISNHASPIDAFFVMW
LAPIGTVGVAKKEVIWYPLLGQLYTLAHHIRIDRSNPAAAIQSMKEAVRVITEKNLSLIM
FPEGTRSGDGRLLPFKKGFVHLALQSHLPIVPMILTGTHLAWRKGTFRVRPVPITVKYLP
PINTDDWTVDKIDDYVKMIHDIYVRNLPASQKPLGSTNRSK
Crambe hispanica subsp. abyssinica FAE GenBank Accession No: AY793549
SEQ ID NO: 84
MTSINVKLLYHYVITNLFNLCFFPLTAIVAGKASRLTIDDLHHLYYSYLQHNVITIAPLFAFTVFGSILY
IVTRPKPVYLVEYSCYLPPTQCRSSISKVMDIFYQVRKADPFRNGTCDDSSWLDFLRKIQERSGLGDETH
GPEGLLQVPPRKTFAAAREETEQVIVGALKNLFENTKVNPKDIGILVVNSSMFNPTPSLSAMVVNTFKLR
SNVRSFNLGGMGCSAGVIAIDLAKDLLHVHKNTYALVVSTENITYNIYAGDNRSMMVSNCLFRVGGAAIL
LSNKPRDRRRSKYELVHTVRTHTGADDKSFRCVQQGDDENGKTGVSLSKDITEVAGRTVKKNIATLGPLI
LPLSEKLLFFVTFMAKKLFKDKVKHYYVPDFKLAIDHFCIHAGGRAVIDVLEKNLGLAPIDVEASRSTLH
RFGNTSSSSIWYELAYIEAKGRMKKGNKVWQIALGSGFKCNSAVWVALSNVKASTNSPWEHCIDRYPVKI
DSDSAKSETRAQNGRS
Lunaria annua FAE GenBank Accession No: ACJ61777
SEQ ID NO: 85
MTSINVKLLYHYVITNFFNLCFFPLTAILAGKASRLTTNDLHHFYSYLQHNLITLTLLFAFTVFGSVLYF
VTRPKPVYLVDYSCYLPPQHLSAGISKTMEIFYQIRKSDPLRNVALDDSSSLDFLRKIQERSGLGDETYG
PEGLFEIPPRKNLASAREETEQVINGALKNLFENTKVNPKEIGILVVNSSMFNPTPSLSAMVVNTFKLRS
NIKSFNLGGMGCSAGVIAIDLAKDLLHVHKNTYALVVSTENITQNIYTGDNRSMMVSNCLFRVGGAAILL
SNKPGDRRRSKYRLAHTVRTHTGADDKSFGCVRQEEDDSGKTGVSLSKDITGVAGITVQKNITTLGPLVL
PLSEKILFVVTRVAKKLLKDKIKHYYVPDFKLAVDHFCIHAGGRAVIDVLEKNLGLSPIDVEASRSTLHR
FGNTSSSSIWYELAYIEAKGRMKKGNKAWQIAVGSGFKCNSAVWVALRNVKASANSPWEHCIHKYPVQMY
SGSSKSETRAQNGRS
AtLPCAT1 NP_172724.2
SEQ ID NO: 86
MDMSSMAGSIGVSVAVLRFLLCFVATIPVSFACRIVPSRLGKHLYAAASGAFLSYLSFGFSSNLHF
LVPMTIGYASMAIYRPKCGIITFFLGFAYLIGCHVFYMSGDAWKEGGIDSTGALMVLTLKVISCSM
NYNDGMLKEEGLREAQKKNRLIQMPSLIEYFGYCLCCGSHFAGPVYEMKDYLEWTEGKGIWDTT
EKRKKPSPYGATIRAILQAAICMALYLYLVPQYPLTRFTEPVYQEWGFLRKFSYQYMAGFTARWK
YYFIWSISEASIIISGLGFSGWTDDASPKPKWDRAKNIVDILGVELAKSAVQIPLVWNIQVSTWLRH
YVYERLVQNGICKAGFFQLLATQTVSAVWHGLYPGYMMFFVQSALMIAGSRVIYRWQQAISPKM
AMLRNIMVFINFLYTVLVLNYSAVGFMVLSLHETLTAYGSVYYIGTIIPVGLILLSYVVPAKPSRPK
PRKEE
AtLPCAT2 NP_176493.1
SEQ ID NO: 87
MELLDMNSMAASIGVSVAVLRFLLCFVATIPISFLWRFIPSRLGKHIYSAASGAFLSYLSFGFSSNL
HFLVPMTIGYASMAMYRPKCGIITFFLGFAYLIGCHVFYMSGDAWKEGGIDSTGALMVLTLKVISC
SINYNDGMLKEEGLREAQKKNRLIQMPSLIEYFGYCLCCGSHFAGPVFEMKDYLEWTEEKGIWA
VSLEKGKRPSPYGAMIRAVFQAAICMALYLYLVPQFPLTRFTEPVYQEWGFLKRFGYQYMAGFTA
RWKYYFIWSISEASIIISGLGFSGWTDETQTKAKWDRAKNVDILGVELAKSAVQIPLFWNIQVSTW
LRHYVYERIVKPGKKAGFFQLLATQTVSAVWHGLYFGYIIFFVQSALMIDGSKAIYRWQQAIPPK
MAMLRNVLVLINFLYTVVVLNYSSVGFMVLSLHETLVAFKSVYYIGTVIPIAVLLLSYLVPVKPVR
PKTRKEE
BrLPCAT S16_Br_Trinity_38655 - ORF 1 (frame 2)
SEQ ID NO: 88
MISMDMDSMAASIGVSVAVLRFLLCFVATIPVSFFWRIVPSRLGKHVYAAASGVFLSYLSFGFSSN
LHFLVPMTIGYASMAMYRPKCGIITFFLGFAYLIGCHVFYMSGDAWKEGGIDSTGALMVLTLKVI
SCAVNYNDGMLKEEGLREAQKKNRLIEMPSLIEYFGYCLCCGSHFAGPVYEMKDYLQWTEGTGI
WDSSEKRKQPSPYLATLRAIFQAGICMALYLYLVPQFPLTRFTEPVYQEWGFWKKFGYQYMAGQ
TARWKYYFIWSISEASIIISGLGFSGWTDDEASPKPKWDRAKNVDILGVELAKSAVQIPLVWNIQV
STWLRHYVYERLVKSGKKAGFFQLLATQTVSAVWHGLYPGYMMFFVQSALMIAGSRVIYRWQQ
AISPKLGVLRSMMVFINFLYTVLVLNYSAVGFMVLSLHETLTAYGSVYYIGTIIPVGLILLSYVVPA
KPYRAKPRKEE
BjLPCAT1 S15_Bj_Trinity_73901 - ORF 1 (frame 3)
SEQ ID NO: 89
MISMDMDSMAASIGVSVAVLRFLLCFVATIPVSFFWRIVPSRLGKHVYAAASGVFLSYLSFGFSSNL
HFLVPMTIGYASMAMYRPKCGIITFFLGFAYLIGCHVFYMSGDAWKEGGIDSTGALMVLTLKVIS
CAVNYNDGMLKEEGLREAQKKNRLIEMPSLIEYFGYCLCCGSHFAGPVYEMKDYLQWTEGTGI
WDSSEKRKQPSPYLATLRAIFQAGICMALYLYLVPQFPLTRFTEPVYQEWGFWKKFGYQYMAGQ
TARWKYYFIWSISEASIIISGLGFSGWTDDEASPKPKWDRAKNVDILGVELAKSAVQIPLVWNIQV
STWLRHYVYERLVKSGKKAGFFQLLATQTVSAVWHGLYPGYMMFFVQSALMIAGSRVIYRWQQ
AISPKLGVLRSMMVFINFLYTVLVLNYSAVGFMVLSLHETLTAYGSVYYIGTIIPVGLILLSYVVPA
KPYRAKPRKEE
BjLPCAT2 _PTX_Sample_S15_Bj_merged_transcripts- ORF 1 (frame 3)
SEQ ID NO: 90
MISMDMDSMAASIGVSVAVLRFLLCFVATIPVSFFWRIVPSRLGKHVYAAASGVFLSYLSFGFSSN
LHFLVPMTIGYASMAMYRPKCGIITFFLGFAYLIGCHVFYMSGDAWKEGGIDSTGALMVLTLKVI
SCAVNYNDGMLKEEGLREAQKKNRLIEMPSLIEYFGYCLCCGSHFAGPVYEMKDYLQWTEGTGI
WDSSEKRKQPSPYLATLRAIFQAGICMALYLYLVPQFPLTRFTEPVYQEWGFWKKFGYQYMAGQ
TARWKYYFIWSISEASIIISGLGFSGWTDDEASPKPKWDRAKNVDILGVELAKSAVQIPLVWNIQV
STWLRHYVYERLVKSGKKAGFFQLLATQTVSAVWHGLYPGYMMFFVQSALMIAGSRVIYRWQQ
AISPKLGVLRSMMVFINFLYTVLVLNYSAVGFMVLSLHETLTAYGSVYYIGTIIPVGLILLSYVVPA
KPYRAKPRKEE
LimdLPCAT1 S03_Ld_Trinity_38978 - ORF 2 (frame 3)
SEQ ID NO: 91
MDLDMDSMASSIGVSVPVLRFLLCYAATIPVSFICRFVPGKTPKNVFSAATGAFLSYLSFGFSSNIH
FLIPMTLGYASMALYRAKCGIVTFFLAFGYLIGCHVYYMSGDAWKEGGIDATGALMVLTLKVISC
SVNYNDGLLKEEGLRPSQKKNRLSSLPSFIEYVGYCLCCGTHFAGPVYEMKDYLEWTAGKGIWA
KSEKAKSPSPFLPALRALLQGAVCMVLYLYLVPQYPLSQFTSPVYQEWGFWKRLSYQYMAGFTA
RWKYYFIWSISEASVILSGLGFSGWTDSSPPKPRWDRAKNVDILGVEFATSGAQVPLVWNIQVST
WLRHYVYDRLVKTGKKPGFFQLLATQTTSAVWHGLYPGYLFFFVQSALMIAGSKVIYRWKQALP
PSASVLQKILVFANFLYTLLVLNYSCVGFMVLSMHETIAAYGSVYYVGTIVPIVLTILGSIIPVKPRR
TKVQKEQ
LimdLPCAT2 S03_Ld_Trinity_29594 - ORF 1 (frame 1)
SEQ ID NO: 92
MNMQNAALLIGVSVPVFRFLVSFLATVPVSFLWRYAPGNLGKHVYAAGSGALLSCLAFGLLSNL
HFLVLMVMGYCSMVFYRSKCGILTFVLGFTYLIGCHFYYMSGDAWKDGGMDATGSLMVLTLKV
ISCAINYNDGLLKEEGLREAQKKNRLINLPSVVEYVGYCLCCGSHFAGPVFEMKDYLQWTKKKGI
WAAKERSPSPYVATIRALLQAAICMVVYMYLVPRFPLSTLAEPIYQEWGFWKKLSYQYITGFSSR
WKYFFVWSISEASMIISGLGFSGWTDTSPQNPQWDRAKNVDILRAELPESAVVLPLVWNIHVSTW
LRHYVYERLIKNGKKPGFFELLATQTVSAVWHGLYPGYIIFFVHTALMIAGSRVIYRWRQAVPPN
MALVKKMLTFMNLLYTVLILNYSYVGFRVLNLHETLAAHRSVYYVGTILPIIFIFLGYIFPAKPSRP
KPRKQQ
pSZ5344; AtPDCT
SEQ ID NO: 93
gctcttctgcttcggattccactacatcaagtgggtgaacctggcgggcgcggaggagggcccccgcccgggcggcattgttagca
accactgcagctacctggacatcctgctgcacatgtccgattccttccccgcctttgtggcgcgccagtcgacggccaagctgccctt
tatcggcatcatcaggtgcgtgaaagtgggggctgctgtggtcgtggtgggcggggtcacaaatgaggacattgatgctgtcgttt
gccgatcaggggagctcgaaagtaagtgcagcctggtcatgggatcacaaatctcaccaccactcgtccaccttgcctgggccttg
cagccaaattatgagctgcctctacgtgaaccgcgaccgctcggggcccaaccacgtgggtgtggccgacctggtgaagcagcgc
atgcaggacgaggccgaggggaagaccccgcccgagtaccggccgctgctcctcttccccgaggtgggcttttgagacactgtttg
tgcttgaaactgtggacgcgcgtgccctgacgcgcctccggcgcctgtctcgcatccattcgcctctcaaccccatctcaccttttctc
catcgccagggcaccacctccaacggcgactacctgcttcccttcaagaccggcgccttcctggccggggtgcccgtccagcccgtg

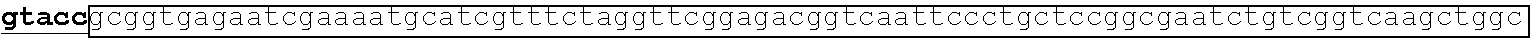

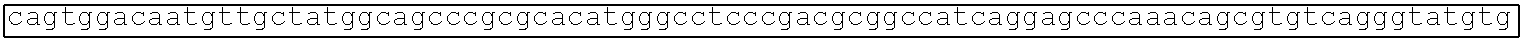

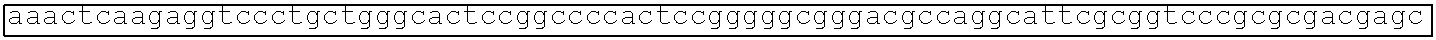

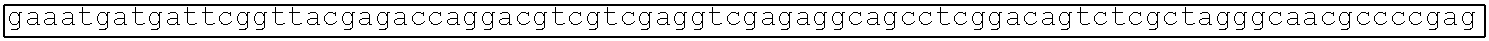

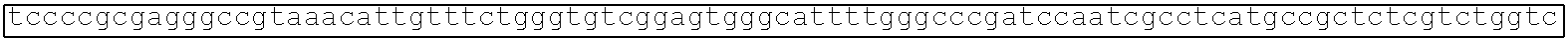

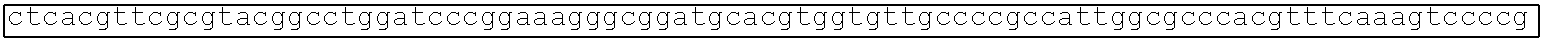

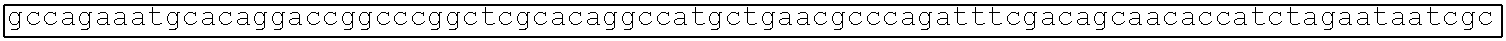

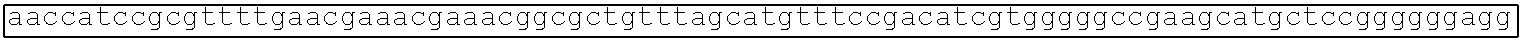

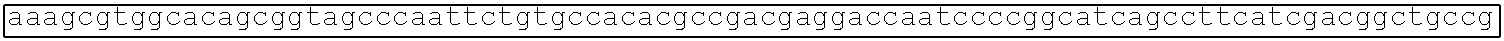

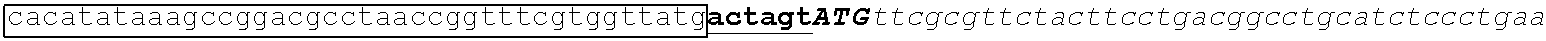

gggcgtgttcggcgtctccccctcctacaacggcctgggcctgacgccccagatgggctgggacaactggaacacgttcgcctg
cgacgtctccgagcagctgctgctggacacggccgaccgcatctccgacctgggcctgaaggacatgggctacaagtacatca
tcctggacgactgctggtcctccggccgcgactccgacggcttcctggtcgccgacgagcagaagttccccaacggcatgggcc
acgtcgccgaccacctgcacaacaactccttcctgttcggcatgtactcctccgcgggcgagtacacgtgcgccggctaccccgg
ctccctgggccgcgaggaggaggacgcccagttcttcgcgaacaaccgcgtggactacctgaagtacgacaactgctacaac
aagggccagttcggcacgcccgagatctcctaccaccgctacaaggccatgtccgacgccctgaacaagacgggccgcccca
tcttctactccctgtgcaactggggccaggacctgaccttctactggggctccggcatcgcgaactcctggcgcatgtccggcga
cgtcacggcggagttcacgcgccccgactcccgctgcccctgcgacggcgacgagtacgactgcaagtacgccggcttccact
gctccatcatgaacatcctgaacaaggccgcccccatgggccagaacgcgggcgtcggcggctggaacgacctggacaacct
ggaggtcggcgtcggcaacctgacggacgacgaggagaaggcgcacttctccatgtgggccatggtgaagtcccccctgatc
atcggcgcgaacgtgaacaacctgaaggcctcctcctactccatctactcccaggcgtccgtccgtcatcgccatcaaccaggactcc
aacggcatccccgccacgcgcgtctggcgctactacgtgtccgacacggacgagtacggccagggcgagatccagatgtggt
ccggccccctggacaacggcgaccaggtcgtggcgctgctgaacggcggctccgtgtcccgccccatgaacacgaccctgga
ggagatcttcttcgactccaacctgggctccaagaagctgacctccacctgggacatctacgacctgtgggcgaaccgcgtcga
caactccacggcgtccgccatcctgggccgcaacaagaccgccaccggcatcctgtacaacgccaccgagcagtcctacaag
gacggcctgtccaagaacgacacccgcctgttcggccagaagatcggctccctgtcccccaacgcgatcctgaacacgaccgt

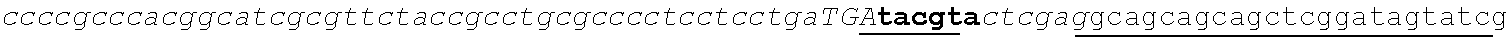

acacactctggacgctggtcgtgtgatggactgttgccgccacacttgctgccttgacctgtgaatatccctgccgcttttatcaaaca
gcctcagtgtgtttgatcttgtgtgtacgcgcttttgcgagttgctagctgcttgtgctatttgcgaataccacccccagcatccccttcc
ctcgtttcatatcgcttgcatcccaaccgcaacttatctacgctgtcctgctatccctcagcgctgctcctgctcctgctcactgcccctc
gcacagccttggtttgggctccgcctgtattctcctggtactgcaacctgtaaaccagcactgcaatgctgatgcacgggaagtagtg

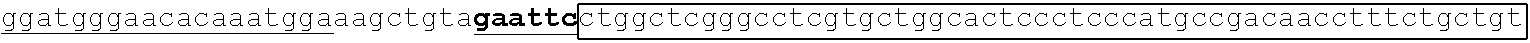

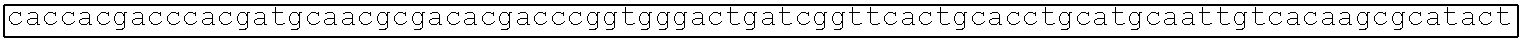

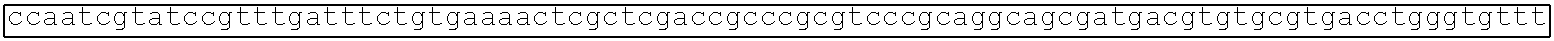

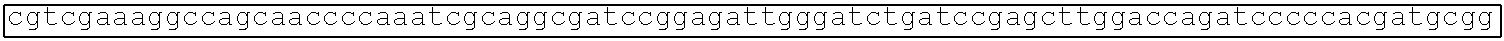

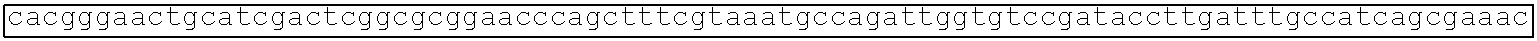

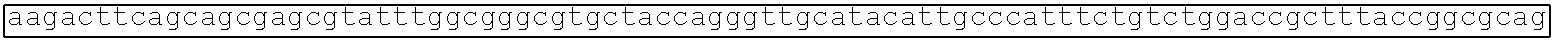

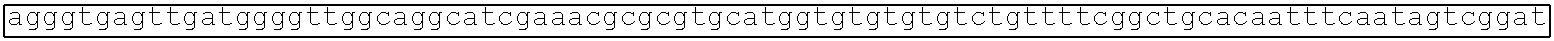

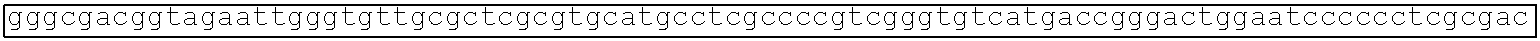

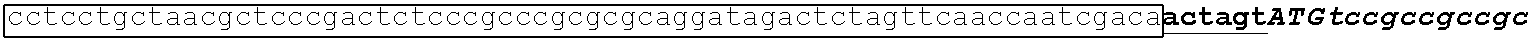

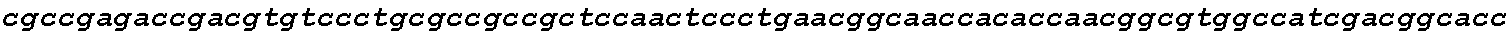

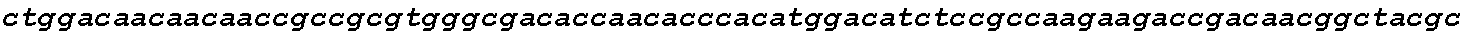

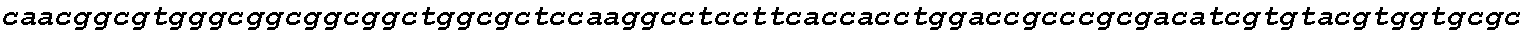

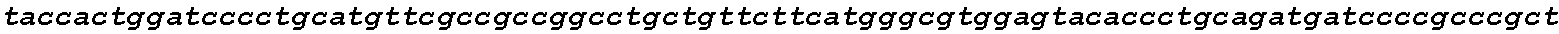

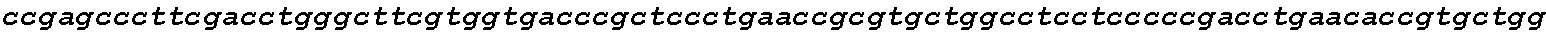

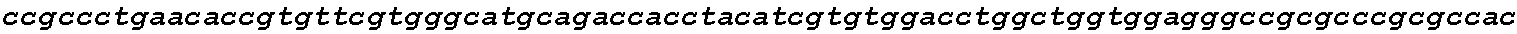

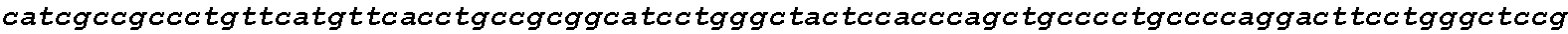

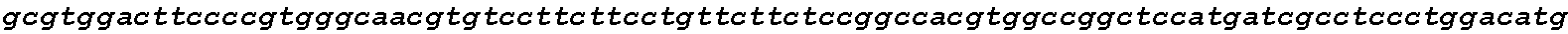

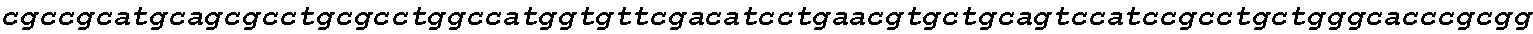

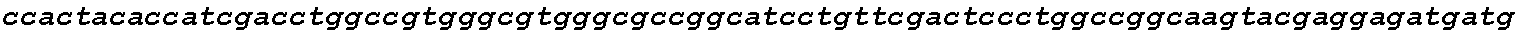

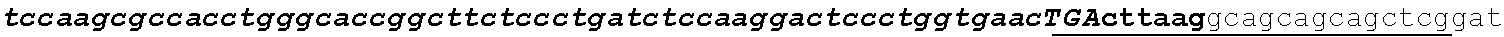

agtatcgacacactctggacgctggtcgtgtgatggactgttgccgccacacttgctgccttgacctgtgaatatccctgccgcttttat
caaacagcctcagtgtgtttgatcttgtgtgtacgcgcttttgcgagttgctagctgcttgtgctatttgcgaataccacccccagcatc
cccttccctcgtttcatatcgcttgcatcccaaccgcaacttatctacgctgtcctgctatccctcagcgctgctcctgctcctgctcact
gcccctcgcacagccttggtttgggctccgcctgtattctcctggtactgcaacctgtaaaccagcactgcaatgctgatgcacggga
agtagtgggatgggaacacaaatggaaagcttaattaagagctccgtcctccactaccacagggtatggtcgtgtggggtcgagc
gtgttgaagcgcagaaggggatgcgccgtcaagatcaggagctaaaaatggtgccagcgaggatccagcgctctcactcttgctg
ccatcgctcccacccttttccccaggggaccctgtggcccacgtgggagacgattccggccaagtggcacatcttcctgatgctctg
ccacccccgccacaaagtgaccgtgatgaaggttaggacaagggtcgggacccgattctggatatgacctctgaggtgtgtttctc
gcgcaagcgtcccccaattcgttacaccacatccctcacaccctcgcccctgacactcgcagttgcccgtgtacgtccccaatgagg
aggaaaaggccgaccccaagctgtacgcccaaaacgtccgcaaagccatggtgcgtcgggaaccgtcaaagtttgcttgcgggt
gggcggggcggctctagcgaattggctcattggccctcaccgaggcagcacatcggacaccagtcgccacccggcttgcatcttcg
ccccctttcttctcgcagatggaggtcgccgggaccaaggacacgacggcggtgtttgaggacaagatgcgctacctgaactccct
gaagagaaagtacggcaagcctgtgcctaagaaaattgagtgaacccccgtcgtcgaccagaagagc
PSZ5295: ATDAG-CPT
SEQ ID NO: 94
gctcttctgcttcggattccactacatcaagtgggtgaacctggcgggcgcggaggagggcccccgcccgggcggcattgttagca
accactgcagctacctggacatcctgctgcacatgtccgattccttccccgcctttgtggcgcgccagtcgacggccaagctgccctt
tatcggcatcatcaggtgcgtgaaagtgggggctgctgtggtcgtggtgggcggggtcacaaatgaggacattgatgctgtcgttt
gccgatcaggggagctcgaaagtaagtgcagcctggtcatgggatcacaaatctcaccaccactcgtccaccttgcctgggccttg
cagccaaattatgagctgcctctacgtgaaccgcgaccgctcggggcccaaccacgtgggtgtggccgacctggtgaagcagcgc
atgcaggacgaggccgaggggaagaccccgcccgagtaccggccgctgctcctcttccccgaggtgggcttttgagacactgtttg
tgcttgaaactgtggacgcgcgtgccctgacgcgcctccggcgcctgtctcgcatccattcgcctctcaaccccatctcaccttttctc
catcgccagggcaccacctccaacggcgactacctgcttcccttcaagaccggcgccttcctggccggggtgcccgtccagcccgtg

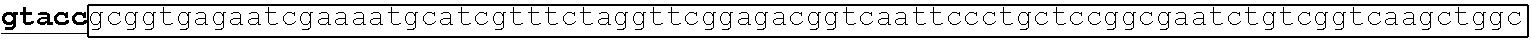

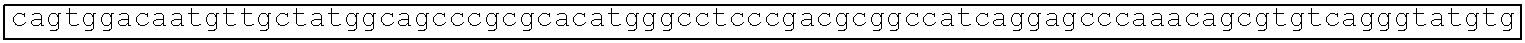

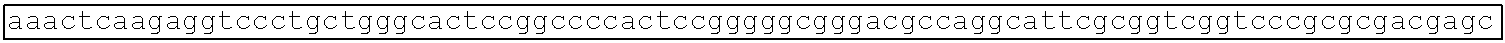

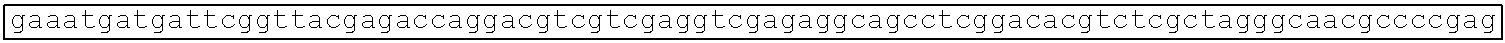

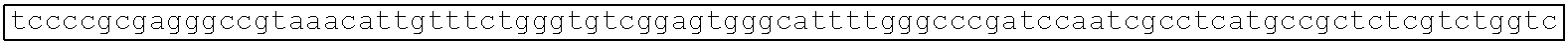

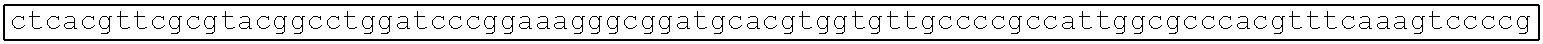

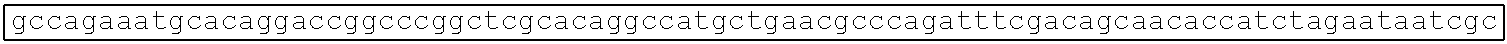

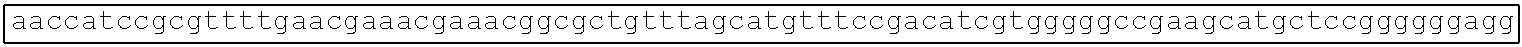

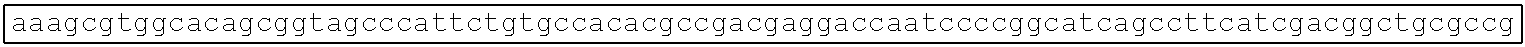

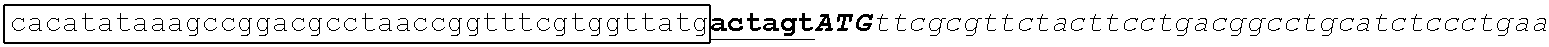

gggcgtgttcggcgtctccccctcctacaacggcctgggcctgacgccccagatgggctgggacaactggaacacgttcgcctg
cgacgtctccgagcagctgctgctggacacggccgaccgcatctccgacctgggcctgaaggacatgggctacaagtacatca
tcctggacgactgctggtcctccggccgcgactccgacggcttcctggtcgccgacgagcagaagttccccaacggcatgggcc
acgtcgccgaccacctgcacaacaactccttcctgttcggcatgtactcctccgcgggcgagtacacgtgcgccggctaccccgg
ctccctgggccgcgaggaggaggacgcccagttcttcgcgaacaaccgcgtggactacctgaagtacgacaactgctacaac
aagggccagttcggcacgcccgagatctcctaccaccgctacaaggccatgtccgacgccctgaacaagacgggccgcccca
tcttctactccctgtgcaactggggccaggacctgaccttctactggggctccggcatcgcgaactcctggcgcatgtccggcga
cgtcacggcggagttcacgcgccccgactcccgctgcccctgcgacggcgacgagtacgactgcaagtacgccggcttccact
gctccatcatgaacatcctgaacaaggccgcccccatgggccagaacgcgggcgtcggcggctggaacgacctggacaacct
ggaggtcggcgtcggcaacctgacggacgacgaggagaaggcgcacttctccatgtgggccatggtgaagtcccccctgatc
atcggcgcgaacgtgaacaacctgaaggcctcctcctactccatctactcccaggcgtccgtcatcgccatcaaccaggactcc
aacggcatccccgccacgcgcgtctggcgctactacgtgtccgacacggacgagtacggccagggcgagatccagatgtggt
ccggccccctggacaacggcgaccaggtcgtggcgctgctgaacggcggctccgtgtcccgccccatgaacacgaccctgga
ggagatcttcttcgactccaacctgggctccaagaagctgacctccacctgggacatctacgacctgtgggcgaaccgcg
caactccacggcgtccgccatcctgggccgcaacaagaccgccaccggcatcctgtacaacgccaccgagcagtcctacaag
gacggcctgtccaagaacgacacccgcctgttcggccagaagatcggctccctgtcccccaacgcgatcctgaacacgaccgt

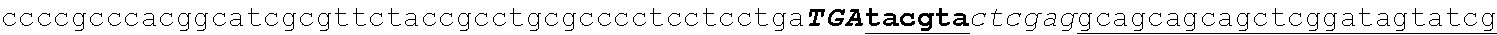

acacactctggacgctggtcgtgtgatggactgttgccgccacacttgctgccttgacctgtgaatatccctgccgcttttatcaaaca
gcctcagtgtgtttgatcttgtgtgtacgcgcttttgcgagttgctagctgcttgtgctatttgcgaataccacccccagcatccccttcc
ctcgtttcatatcgcttgcatcccaaccgcaacttatctacgctgtcctgctatccctcagcgctgctcctgctcctgctcactgcccctc
gcacagccttggtttgggctccgcctgtattctcctggtactgcaacctgtaaaccagcactgcaatgctgatgcacgggaagtagtg

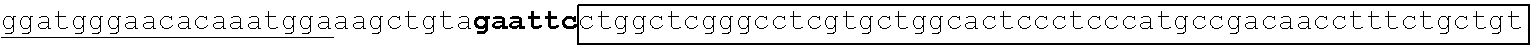

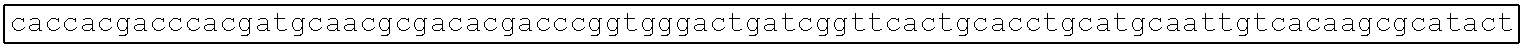

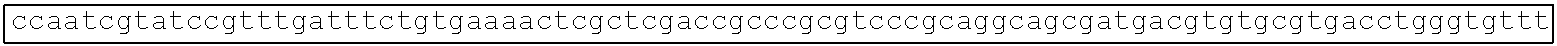

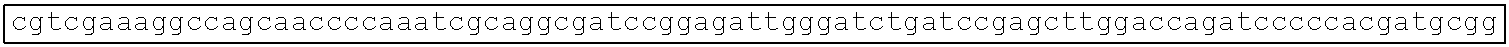

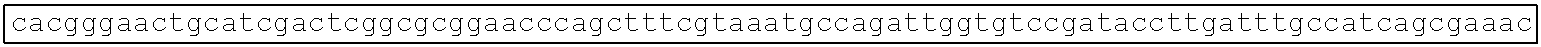

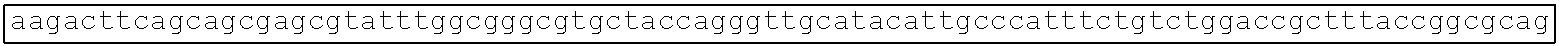

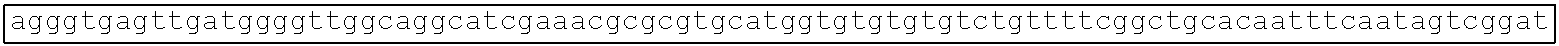

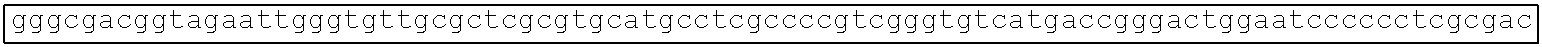

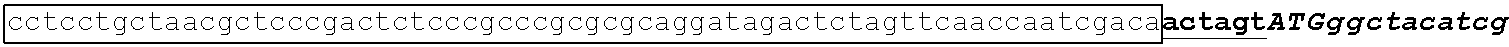

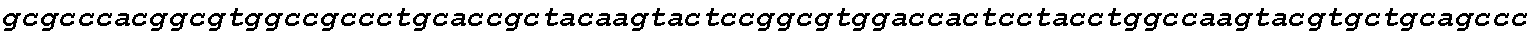

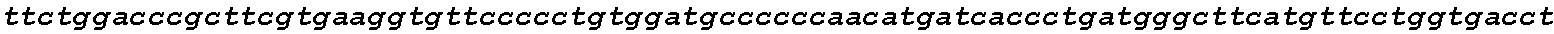

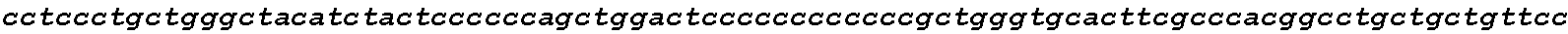

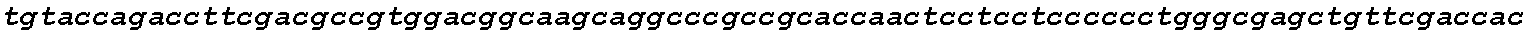

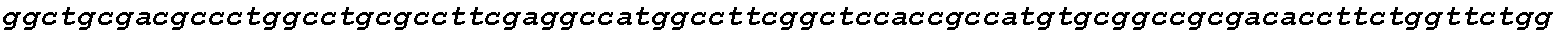

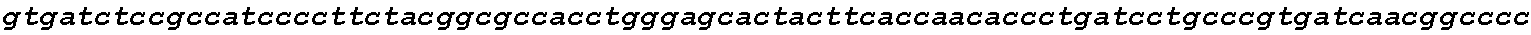

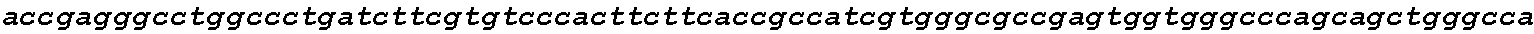

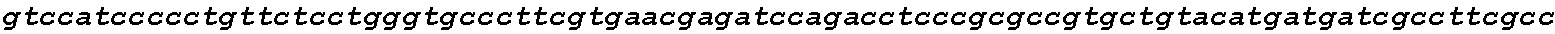

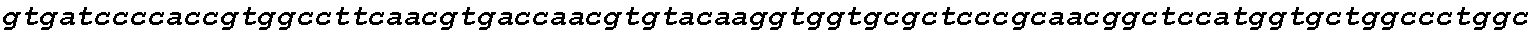

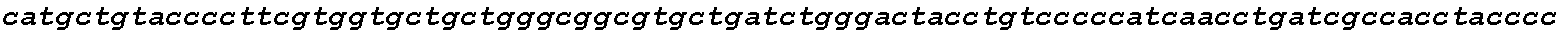

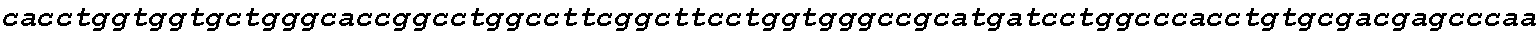

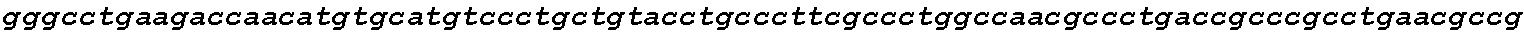

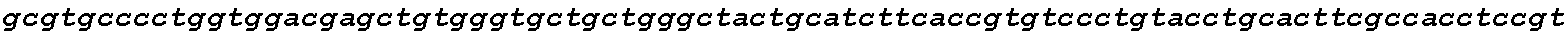

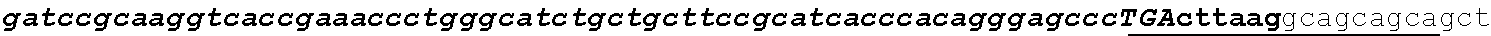

cggatagtatcgacacactctggacgctggtcgtgtgatggactgttgccgccacacttgctgccttgacctgtgaatatccctgccgc
ttttatcaaacagcctcagtgtgtttgatcttgtgtgtacgcgcttttgcgagttgctagctgcttgtgctatttgcgaataccaccccca
gcatccccttccctcgtttcatatcgcttgcatcccaaccgcaacttatctacgctgtcctgctatccctcagcgctgctcctgctcctgc
tcactgcccctcgcacagccttggtttgggctccgcctgtattctcctggtactgcaacctgtaaaccagcactgcaatgctgatgcac
gggaagtagtgggatgggaacacaaatggaaagcttaattaagagctccgtcctccactaccacagggtatggtcgtgtggggtc
gagcgtgttgaagcgcagaaggggatgcgccgtcaagatcaggagctaaaaatggtgccagcgaggatccagcgctctcactct
tgctgccatcgctcccacccttttccccaggggaccctgtggcccacgtgggagacgattccggccaagtggcacatcttcctgatg
ctctgccacccccgccacaaagtgaccgtgatgaaggttaggacaagggtcgggacccgattctggatatgacctctgaggtgtgt
ttctcgcgcaagcgtcccccaattcgttacaccacatccctcacaccctcgcccctgacactcgcagttgcccgtgtacgtccccaat
gaggaggaaaaggccgaccccaagctgtacgcccaaaacgtccgcaaagccatggtgcgtcgggaaccgtcaaagtttgcttgc
gggtgggcggggcggctctagcgaattggctcattggccctcaccgaggcagcacatcggacaccagtcgccacccggcttgcat
cttcgccccctttcttctcgcagatggaggtcgccgggaccaaggacacgacggcggtgtttgaggacaagatgcgctacctgaac
tccctgaagagaaagtacggcaagcctgtgcctaagaaaattgagtgaacccccgtcgtcgaccagaagagc
BrDAG-CPT in pSZ5345 and pSZ5350
SEQ ID NO: 95

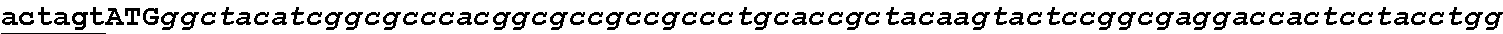

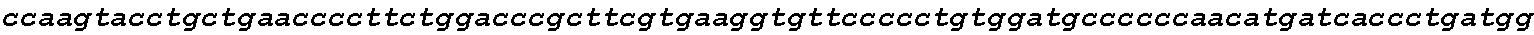

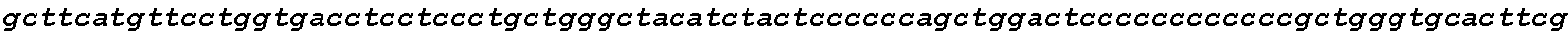

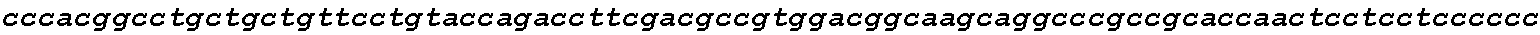

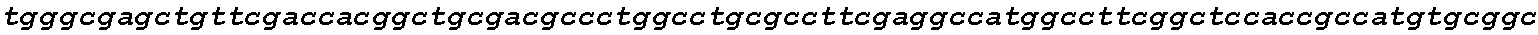

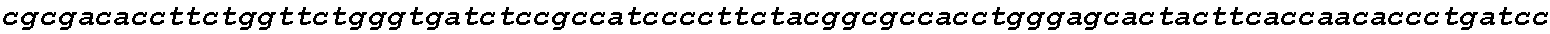

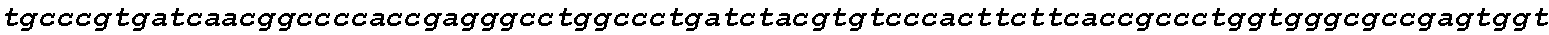

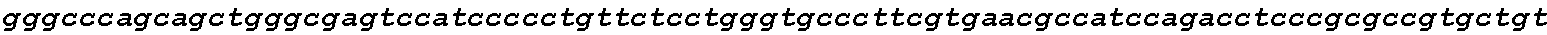

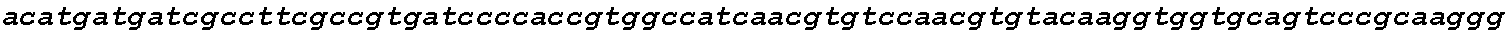

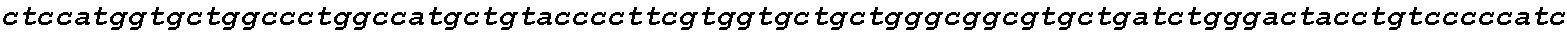

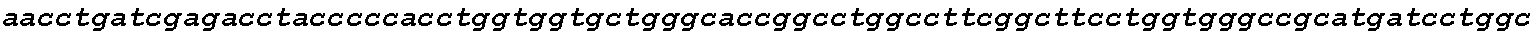

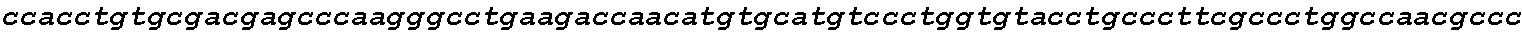

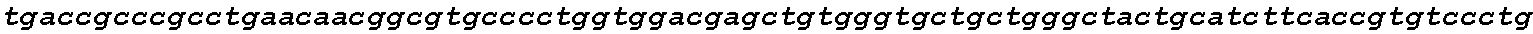

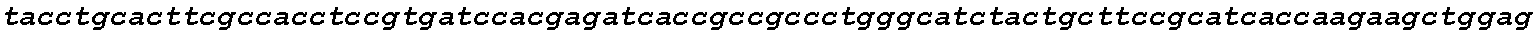

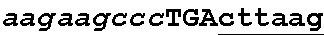

BjDAG-CPT in pSZ5306 and pSZ5347
SEQ ID NO: 96

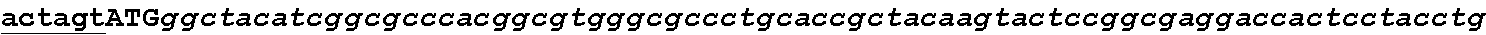

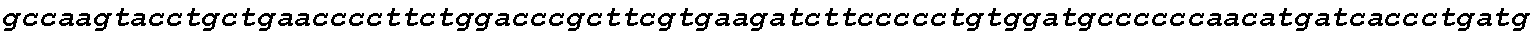

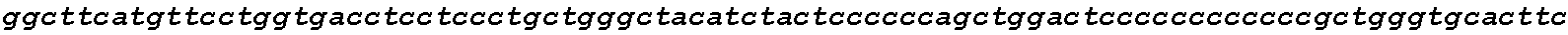

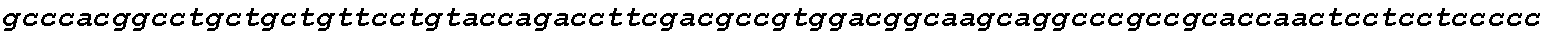

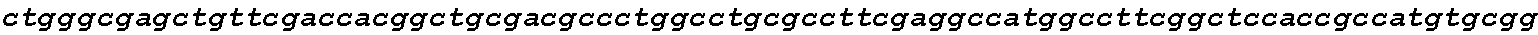

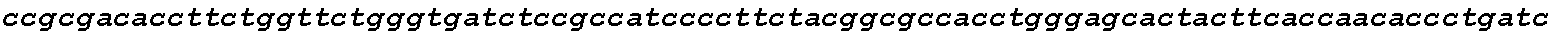

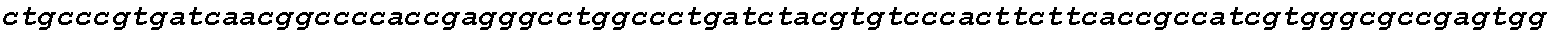

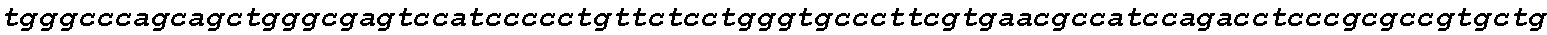

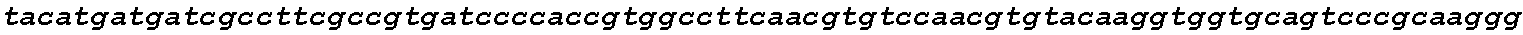

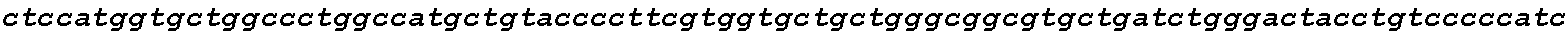

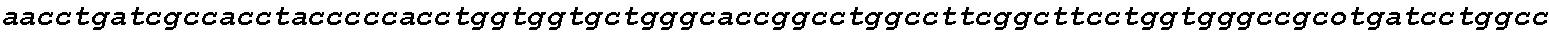

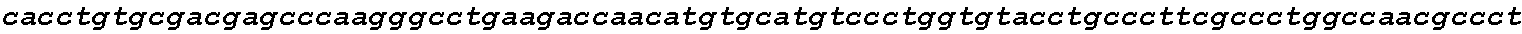

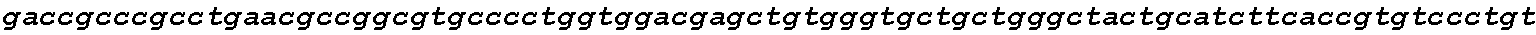

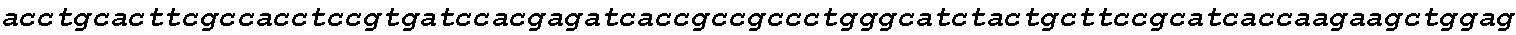

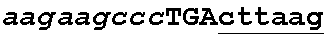

PSZ5296; AtLPCAT1
SEQ ID NO: 97
gctcttctgcttcggattccactacatcaagtgggtgaacctggcgggcgcggaggagggcccccgcccgggcggcattgttagca
accactgcagctacctggacatcctgctgcacatgtccgattccttccccgcctttgtggcgcgccagtcgacggccaagctgccctt
tatcggcatcatcaggtgcgtgaaagtgggggctgctgtggtcgtggtgggcggggtcacaaatgaggacattgatgctgtcgttt
gccgatcaggggagctcgaaagtaagtgcagcctggtcatgggatcacaaatctcaccaccactcgtccaccttgcctgggccttg
cagccaaattatgagctgcctctacgtgaaccgcgaccgctcggggcccaaccacgtgggtgtggccgacctggtgaagcagcgc
atgcaggacgaggccgaggggaagaccccgcccgagtaccggccgctgctcctcttccccgaggtgggcttttgagacactgtttg
tgcttgaaactgtggacgcgcgtgccctgacgcgcctccggcgcctgtctcgcatccattcgcctctcaaccccatctcaccttttctc
catcgccagggcaccacctccaacggcgactacctgcttcccttcaagaccggcgccttcctggccggggtgcccgtccagcccgtg

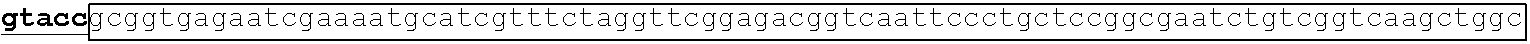

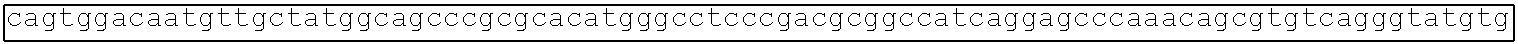

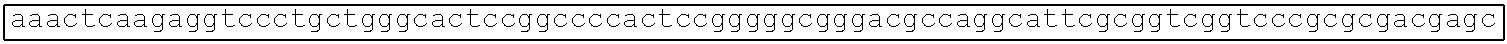

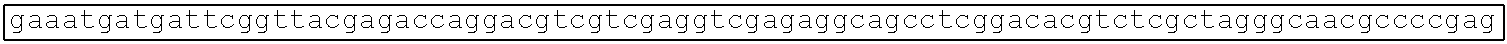

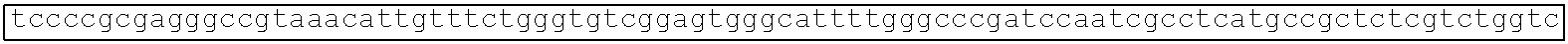

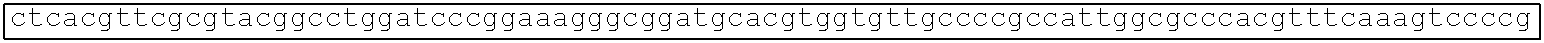

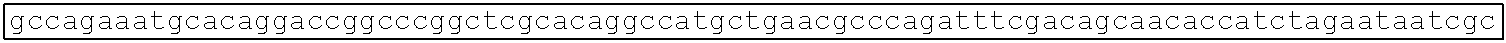

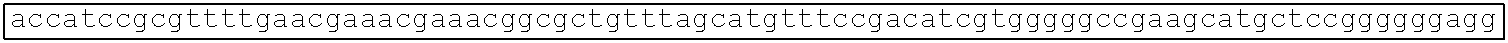

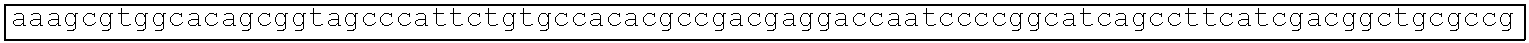

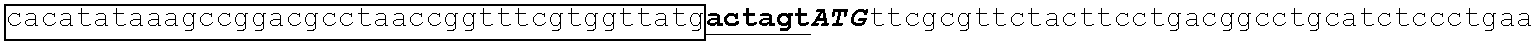

gggcgtgttcggcgtctccccctcctacaacggcctgggcctgacgccccagatgggctgggacaactggaacacgttcgcctg
cgacgtctccgagcagctgctgctggacacggccgaccgcatctccgacctgggcctgaaggacatgggctacaagtacatca
tcctggacgactgctggtcctccggccgcgactccgacggcttcctggtcgccgacgagcagaagttccccaacggcacatgggcc
acgtcgccgaccacctgcacaacaactccttcctgttcggcatgtactcctccgcgggcgagtacacgtgcgccggctaccccgg
ctccctgggccgcgaggaggaggacgcccagttcttcgcgaacaaccgcgtggactacctgaagtacgacaactgctacaac
aagggccagttcggcacgcccgagatctcctaccaccgctacaaggccatgtccgacgccctgaacaagacgggccgcccca
tcttctactccctgtgcaactggggccaggacctgaccttctactggggctccggcatcgcgaactcctggcgcatgtccggcga
cgtcacggcggagttcacgcgccccgactcccgctgcccctgcgacggcgacgagtacgactgcaagtacgccggcttccact
gctccatcatgaacatcctgaacaaggccgcccccatgggccagaacgcgggcgtcggcggctggaacgacctggacaacct
ggaggtcggcgtcggcaacctgacggacgacgaggagaaggcgcacttctccatgtgggccatggtgaagtcccccctgatc
atcggcgcgaacgtgaacaacctgaaggcctcctcctactccatctactcccaggcgtccgtcatcgccatcaaccaggactcc
aacggcatccccgccacgcgcgtctggcgctactacgtgtccgacacggacgagtacggccagggcgagatccagatgtggt
ccggccccctggacaacggcgaccaggtcgtggcgctgctgaacggcggctccgtgtcccgccccatgaacacgaccctgga
ggagatcttcttcgactccaacctgggctccaagaagctgacctccacctgggacatctacgacctgtgggcgaaccgcgtcga
caactccacggcgtccgccatcctgggccgcaacaagaccgccaccggcatcctgtacaacgccaccgagcagtcctacaag
gacggcctgtccaagaacgacacccgcctgttcggccagaagatcggctccctgtcccccaacgcgatcctgaacacgaccgt

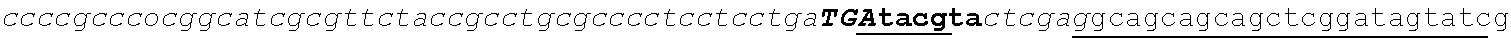

acacactctggacgctggtcgtgtgatggactgttgccgccacacttgctgccttgacctgtgaatatccctgccgcttttatcaaaca
gcctcagtgtgtttgatcttgtgtgtacgcgcttttgcgagttgctagctgcttgtgctatttgcgaataccacccccagcatccccttcc
ctcgtttcatatcgcttgcatcccaaccgcaacttatctacgctgtcctgctatccctcagcgctgctcctgctcctgctcactgcccctc
gcacagccttggtttgggctccgcctgtattctcctggtactgcaacctgtaaaccagcactgcaatgctgatgcacgggaagtagtg

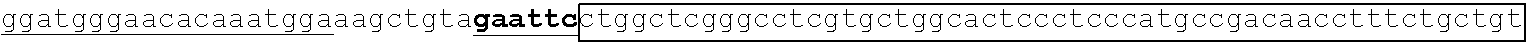

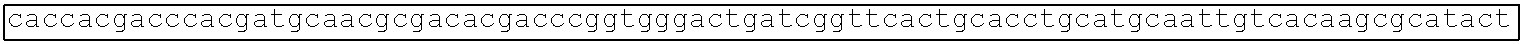

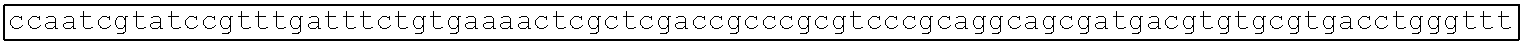

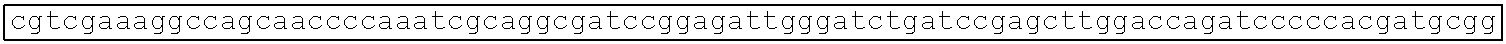

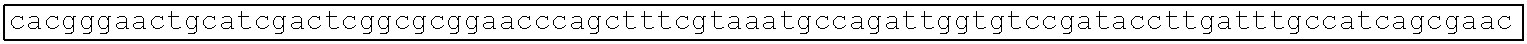

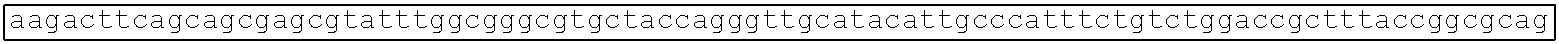

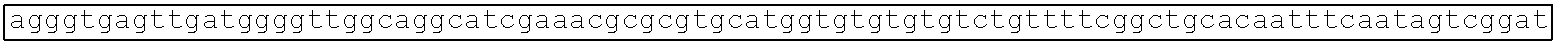

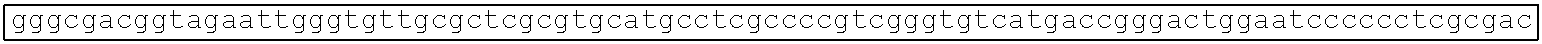

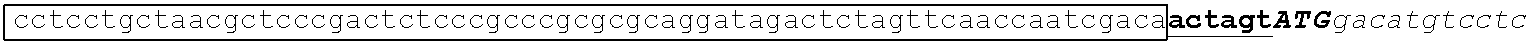

catggccggctccatcggcgtgtccgtggccgtgctgcgcttcctgctgtgcttcgtggccaccatccccgtgtccttcgcctgccg
catcgtgccctcccgcctgggcaagcacctgtacgccgccgcctccggcgccttcctgtcctacctgtccttcggcttctcctccaac
ctgcacttcctggtgcccatgaccatcggctacgcctccatggccatctaccgccccaagtgcggcatcatcaccttcttcctgggc
ttcgcctacctgatcggctgccacgtgttctacatgtccggcgacgcctggaaggagggcggcatcgactccaccggcgccctg
atggtgctgaccctgaaggtgatctcctgctccatgaactacaacgacggcatgctgaaggaggagggcctgcgcgaggccc
agaagaagaaccgcctgatccagatgccctccctgatcgagtacttcggctactgcctgtgctgcggctcccacttcgccggccc
cgtgtacgagatgaaggactacctggagtggaccgagggcaagggcatctgggacaccaccgagaagcgcaagaagccct
ccccctacggcgccaccatccgcgccatcctgcaggccgccatctgcatggccctgtacctgtacctggtgccccagtaccccctg
acccgcttcaccgagcccgtgtaccaggagtggggcttcctgcgcaagttctcctaccagtacatggccggcttcaccgcccgct
ggaagtactacttcatctggtccatctccgaggcctccatcatcatctccggcctgggcttctccggctggaccgacgacgcctcc
cccaagcccaagtgggaccgcgccaagaacgtggacatcctgggcgtggagctggccaagtccgccgtgcagatccccctgg
tgtggaacatccaggtgtccacctggctgcgccactacgtgtacgagcgcctggtgcagaacggcaagaaggccggcttcttc
cagctgctggccacccagaccgtgtccgccgtgtggcacggcctgtaccccggctacatgatgttcttcgtgcagtccgccctga
tgatcgccggctcccgcgtgatctaccgctggcagcaggccatctcccccaagatggccatgctgcgcaacatcatggtgttcat
caacttcctgtacaccgtgctggtgctgaactactccgccgtgggcttcatggtgctgtccctgcacgagaccctgaccgcctacg
gctccgtgtactacatcggcaccatcatccccgtgggcctgatcctgctgtcctacgtggtgcccgccaagccctcccgccccaag

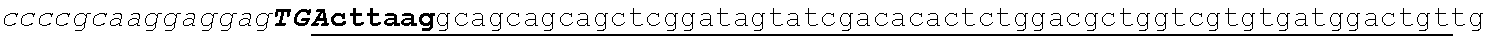

ccgccacacttgctgccttgacctgtgaatatccctgccgcttttatcaaacagcctcagtgtgtttgatcttgtgtgtacgcgcttttgc
gagttgctagctgcttgtgctatttgcgaataccacccccagcatccccttccctcgtttcatatcgcttgcatcccaaccgcaacttat
ctacgctgtcctgctatccctcagcgctgctcctgctcctgctcactgcccctcgcacagccttggtttgggctccgcctgtattctcctg
gtactgcaacctgtaaaccagcactgcaatgctgatgcacgggaagtagtgggatgggaacacaaatggaaagcttaattaagag
ctccgtcctccactaccacagggtatggtcgtgtggggtcgagcgtgttgaagcgcagaaggggatgcgccgtcaagatcaggag
ctaaaaatggtgccagcgaggatccagcgctctcactcttgctgccatcgctcccacccttttccccaggggaccctgtggcccacg
tgggagacgattccggccaagtggcacatcttcctgatgctctgccacccccgccacaaagtgaccgtgatgaaggttaggacaa
gggtcgggacccgattctggatatgacctctgaggtgtgtttctcgcgcaagcgtcccccaattcgttacaccacatccctcacacc
ctcgcccctgacactcgcagttgcccgtgtacgtccccaatgaggaggaaaaggccgaccccaagctgtacgcccaaaacgtccg
caaagccatggtgcgtcgggaaccgtcaaagtttgcttgcgggtgggcggggcggctctagcgaattggctcattggccctcaccg
aggcagcacatcggacaccagtcgccacccggcttgcatcttcgccccctttcttctcgcagatggaggtcgccgggaccaaggac
acgacggcggtgtttgaggacaagatgcgctacctgaactccctgaagagaaagtacggcaagcctgtgcctaagaaaattgag
tgaacccccgtcgtcgaccagaagagcgctcttctgcttcggattccactacatcaagtgggtgaacctggcgggcgcgga
ggagggcccccgcccgggcggcattgttagcaaccactgcagctacctggacatcctgctgcacatgtccgattccttc
cccgcctttgtggcgcgccagtcgacggccaagctgccctttatcggcatcatcaggtgcgtgaaagtgggggctgctg
tggtcgtggtgggcggggtcacaaatgaggacattgatgctgtcgtttgccgatcaggggagctcgaaagtaagtgca
gcctggtcatgggatcacaaatctcaccaccactcgtccaccttgcctgggccttgcagccaaattatgagctgcctcta
cgtgaaccgcgaccgctcggggcccaaccacgtgggtgtggccgacctggtgaagcagcgcatgcaggacgaggcc
gaggggaagaccccgcccgagtaccggccgctgctcctcttccccgaggtgggcttttgagacactgtttgtgcttgaa
actgtggacgcgcgtgccctgacgcgcctccggcgcctgtctcgcatccattcgcctctcaaccccatctcaccttttctc
catcgccagggcaccacctccaacggcgactacctgcttcccttcaagaccggcgccttcctggccggggtgcccgtcc

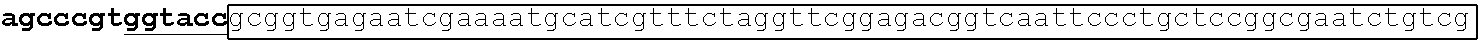

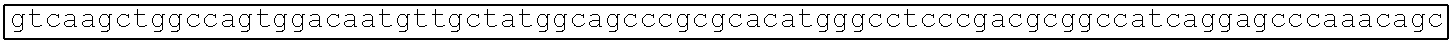

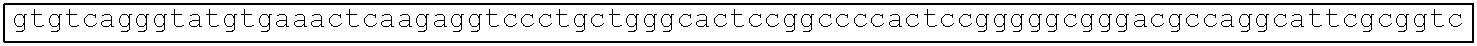

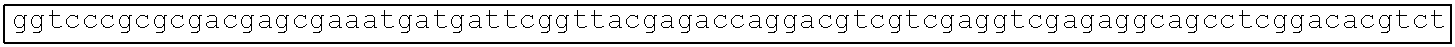

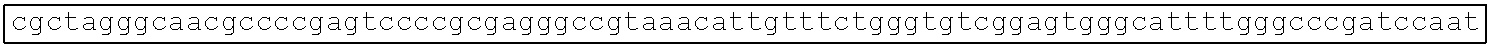

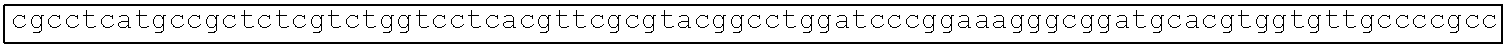

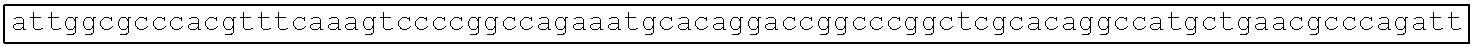

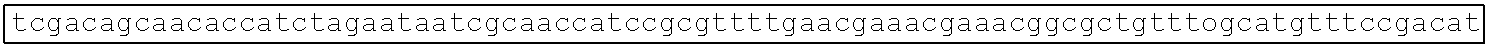

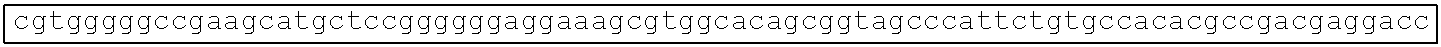

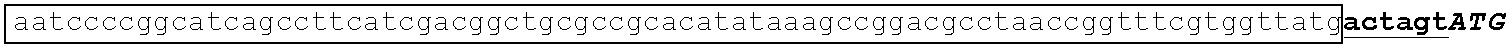

ttcgcgactacacctgacggcctgcatctccctgaagggcgtgttcggcgtctccccctcctacaacggcctgggcctga
cgccccagatgggctgggacaactggaacacgttcgcctgcgacgtctccgagcagctgctgctggacacggccgaccgc
atctccgacctgggcctgaaggacatgggctacaagtacatcatcctggacgactgctggtcctccggccgcgactccgac
ggcttcctggtcgccgacgagcagaagttccccaacggcatgggccacgtcgccgaccacctgcacaacaactccacctgt
tcggcatgtactcctccgcgggcgagtacacgtgcgccggctaccccggctccctgggccgcgaggaggaggacgcccag
acacgcgaacaaccgcgtggactacctgaagtacgacaactgctacaacaagggccagttcggcacgcccgagatctcc
taccaccgctacaaggccatgtccgacgccctgaacaagacgggccgccccatcttctactccctgtgcaactggggccag
gacctgaccttctactggggctccggcatcgcgaactcctggcgcatgtccggcgacgtcacggcggagttcacgcgcccc
gactcccgctgcccctgcgacggcgacgagtacgactgcaagtacgccggcttccactgctccatcatgaacatcctgaac
aaggccgcccccatgggccagaacgcgggcgtcggcggctggaacgacctggacaacctggaggtcggcgtcggcaac
ctgacggacgacgaggagaaggcgcacttctccatgtgggccatggtgaagtcccccctgatcatcggcgcgaacgtgaa
caacctgaaggcctcctcctactccatctactcccaggcgtccgtcatcgccatcaaccaggactccaacggcatccccgcca
cgcgcgtctggcgctactacgtgtccgacacggacgagtacggccagggcgagatccagatgtggtccggccccctggac
aacggcgaccaggtcgtggcgctgctgaacggcggctccgtgtcccgccccatgaacacgaccctggaggagatcacac
gactccaacctgggctccaagaagctgacctccacctgggacatctacgacctgtgggcgaaccgcgtcgacaactccacg
gcgtccgccatcctgggccgcaacaagaccgccaccggcatcctgtacaacgccaccgagcagtcctacaaggacggcct
gtccaagaacgacacccgcctgttcggccagaagatcggctccctgtcccccaacgcgatcctgaacacgaccgtccccgc

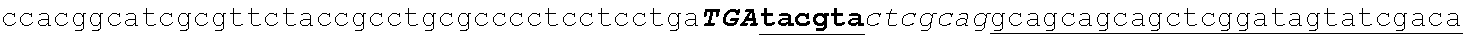

cactctggacgctggtcgtgtgatggactgttgccgccacacttgctgccttgacctgtgaatatccctgccgcttttatcaaaca
gcctcagtgtgtttgatcttgtgtgtacgcgcttttgcgagttgctagctgcttgtgctatttgcgaataccacccccagcatcccct
tccctcgtttcatatcgcttgcatcccaaccgcaacttatctacgctgtcctgctatccctcagcgctgctcctgctcctgctcactg
cccctcgcacagccttggtttgggctccgcctgtattctcctggtactgcaacctgtaaaccagcactgcaatgctgatgcacggg

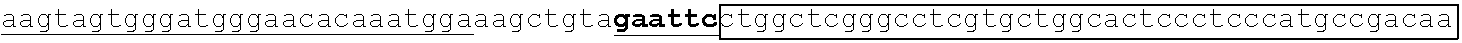

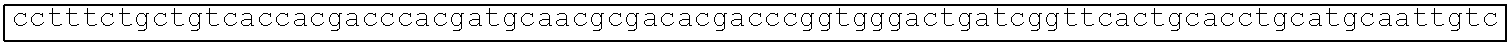

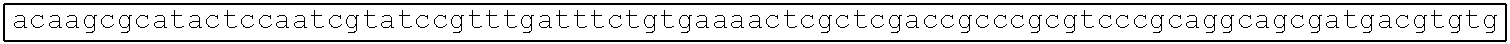

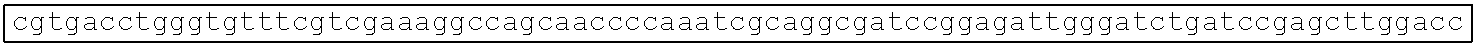

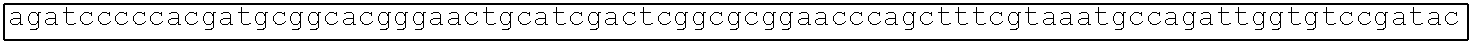

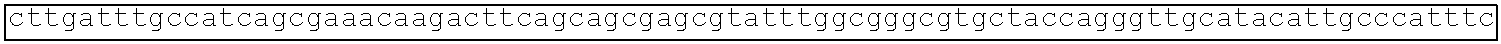

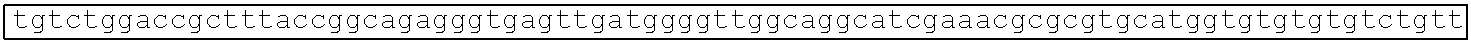

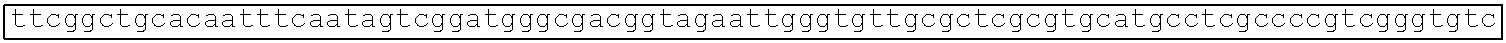

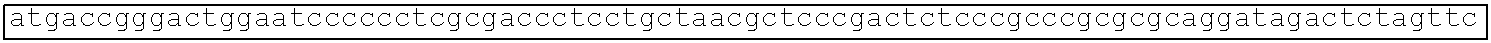

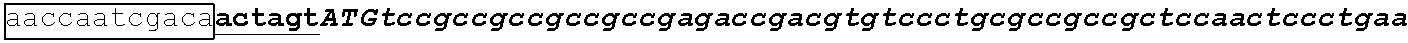

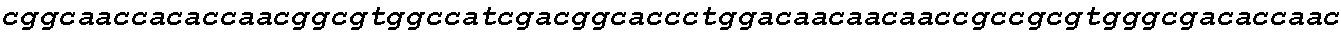

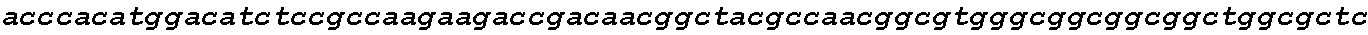

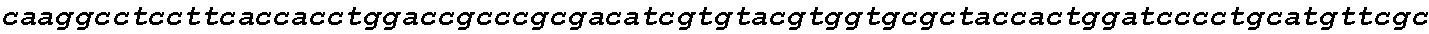

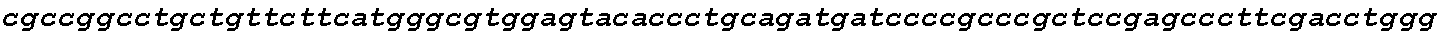

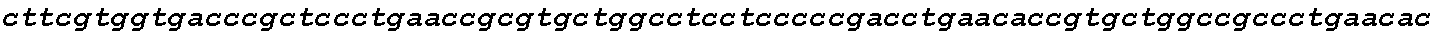

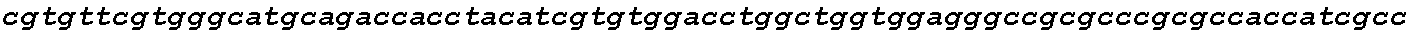

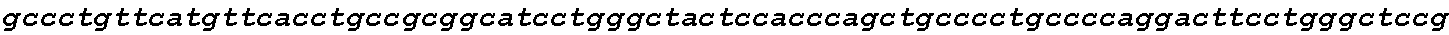

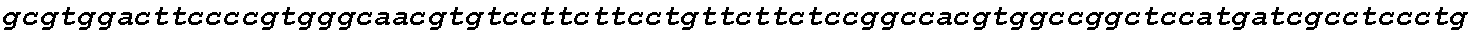

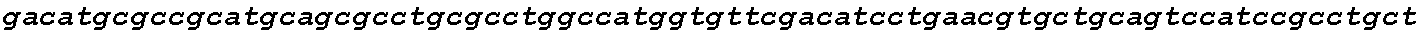

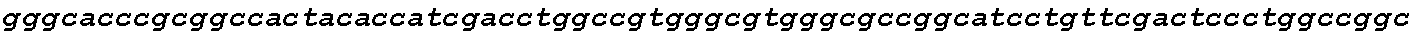

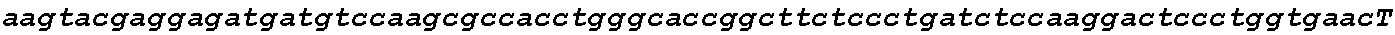

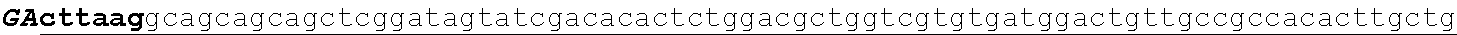

ccttgacctgtgaatatccctgccgcttttatcaaacagcctcagtgtgtttgatcttgtgtgtacgcgcttttgcgagttgctagct
gcttgtgctatttgcgaataccacccccagcatccccttccctcgtttcatatcgcttgcatcccaaccgcaacttatctacgctgtc
ctgctatccctcagcgctgctcctgctcctgctcactgcccctcgcacagccttggtttgggctccgcctgtattctcctggtactgc
aacctgtaaaccagcactgcaatgctgatgcacgggaagtagtgggatgggaacacaaatggaaagcttaattaagagctcc
gtcctccactaccacagggtatggtcgtgtggggtcgagcgtgttgaagcgcagaaggggatgcgccgtcaagatcag
gagctaaaaatggtgccagcgaggatccagcgctctcactcttgctgccatcgctcccacccttttccccaggggaccc
tgtggcccacgtgggagacgattccggccaagtggcacatcttcctgatgctctgccacccccgccacaaagtgaccgt
gatgaaggttaggacaagggtcgggacccgattctggatatgacctctgaggtgtgtttctcgcgcaagcgtcccccaa
ttcgttacaccacatccctcacaccctcgcccctgacactcgcagttgcccgtgtacgtccccaatgaggaggaaaagg
ccgaccccaagctgtacgcccaaaacgtccgcaaagccatggtgcgtcgggaaccgtcaaagtttgcttgcgggtggg
cggggcggctctagcgaattggctcattggccctcaccgaggcagcacatcggacaccagtcgccacccggcttgcat
cttcgccccctttcttctcgcagatggaggtcgccgggaccaaggacacgacggcggtgtttgaggacaagatgcgct
acctgaactccctgaagagaaagtacggcaagcctgtgcctaagaaaattgagtgaacccccgtcgtcgaccagaag
agc
AtLPCAT2
SEQ ID NO: 98

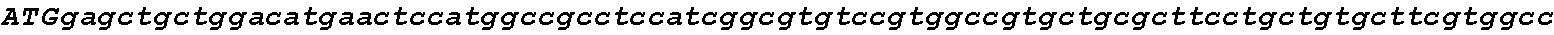

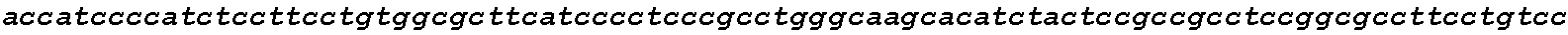

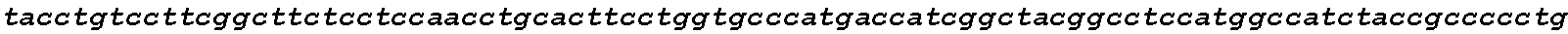

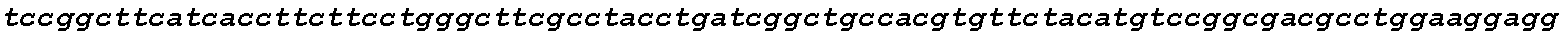

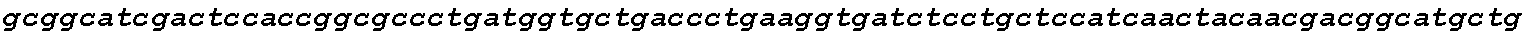

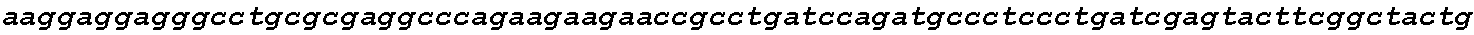

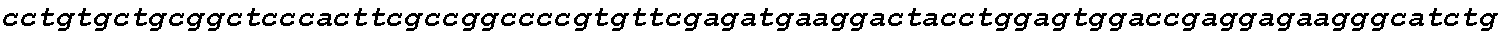

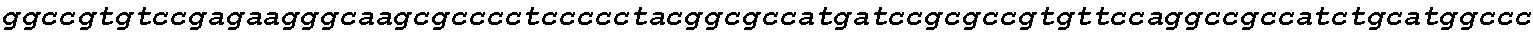

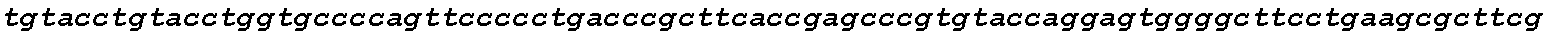

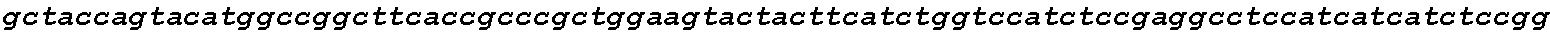

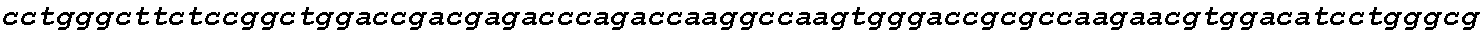

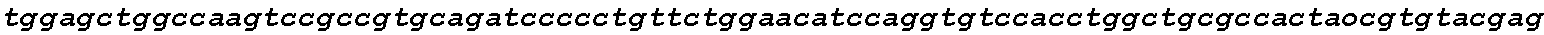

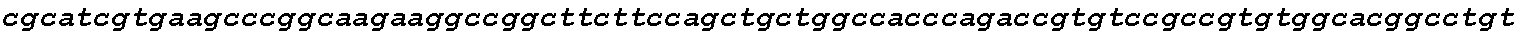

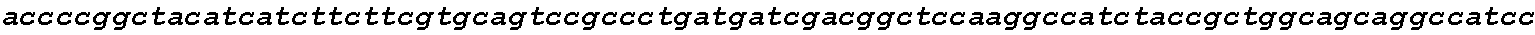

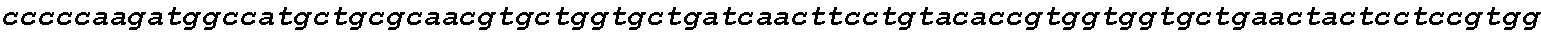

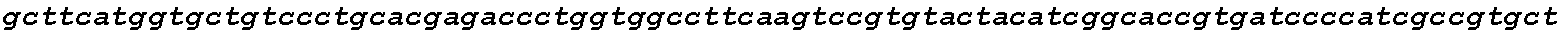

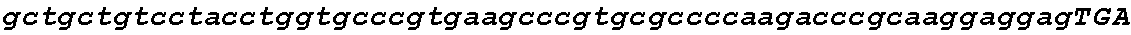

BrLPCAT
SEQ ID NO: 99

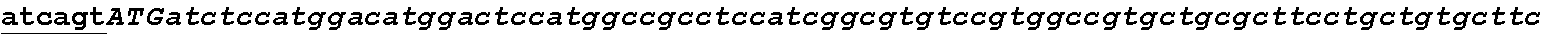

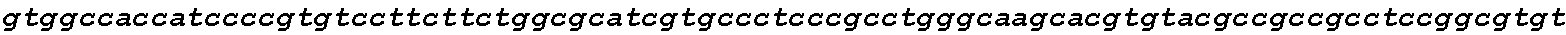

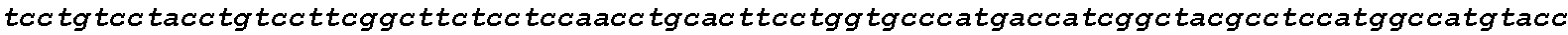

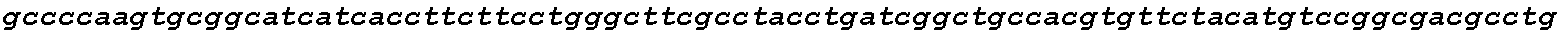

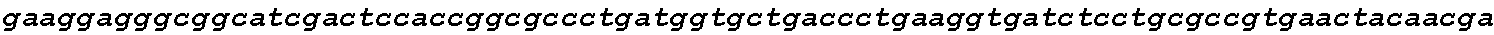

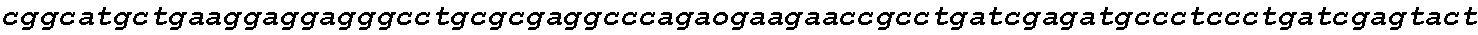

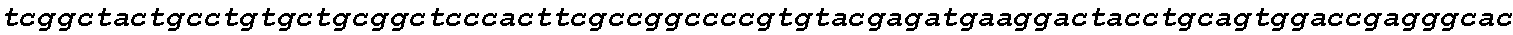

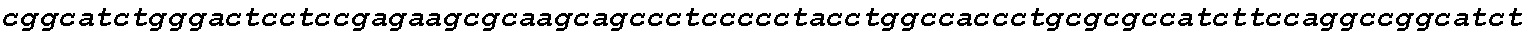

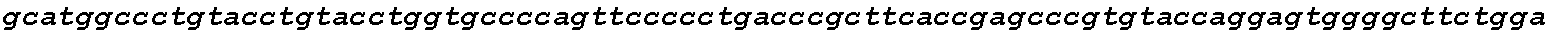

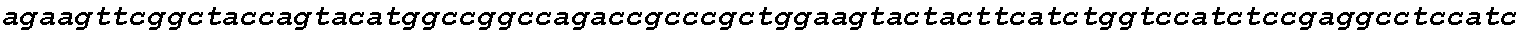

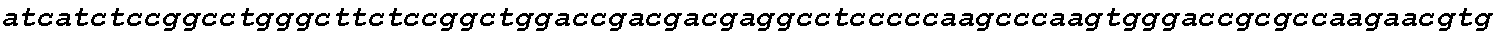

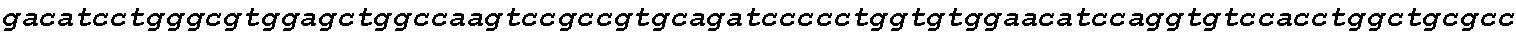

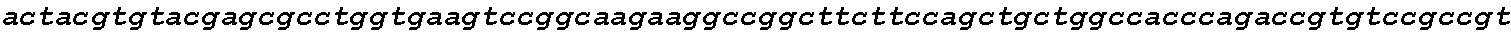

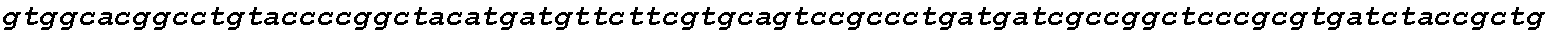

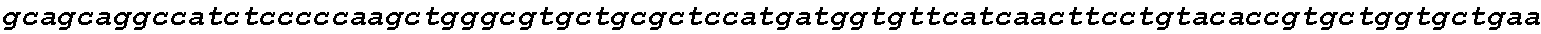

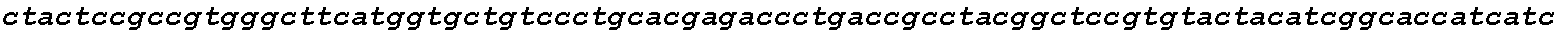

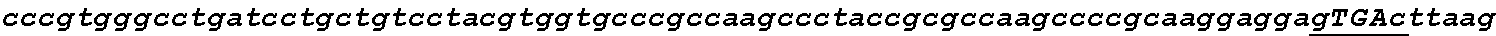

BjLPCAT
SEQ ID NO: 100

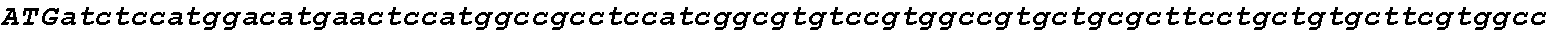

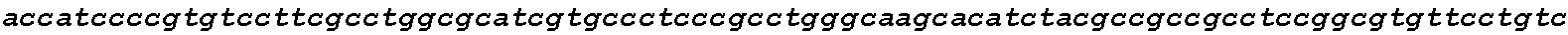

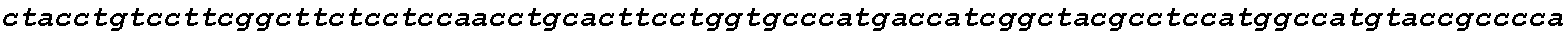

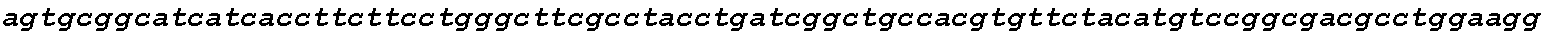

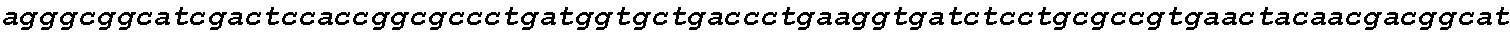

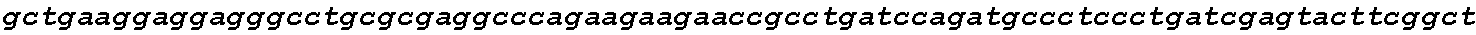

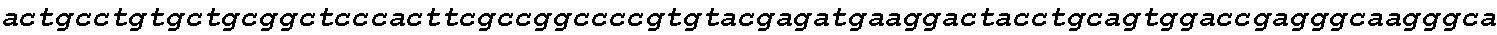

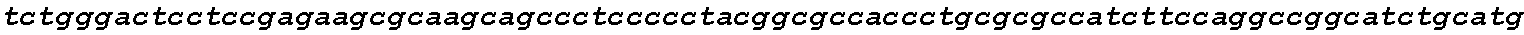

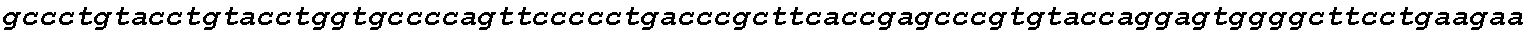

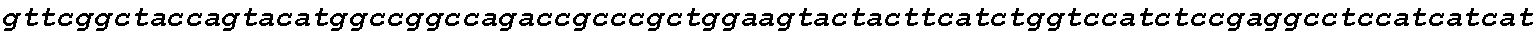

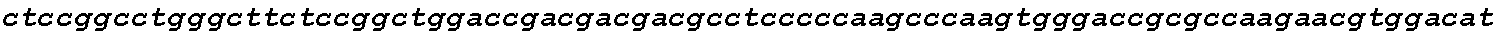

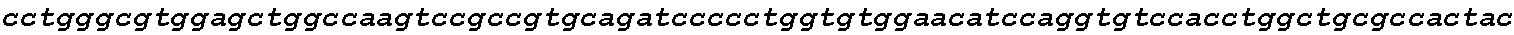

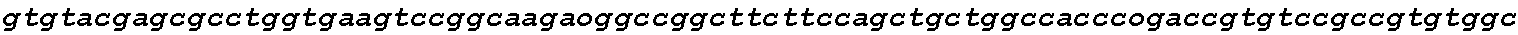

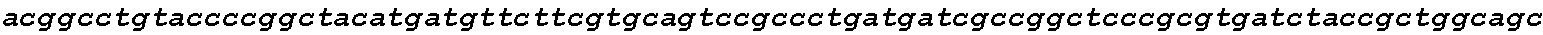

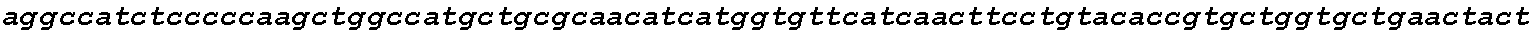

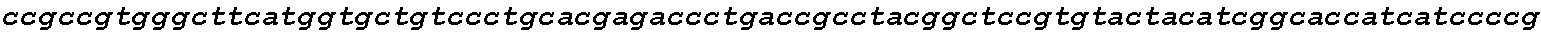

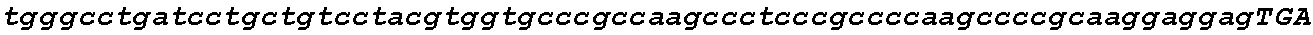

LimdLPCAT1
SEQ ID NO: 101

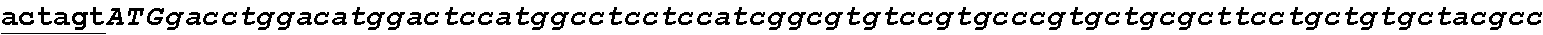

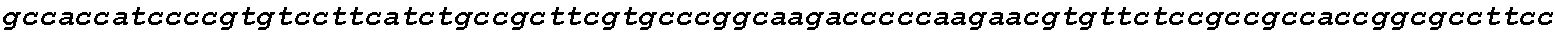

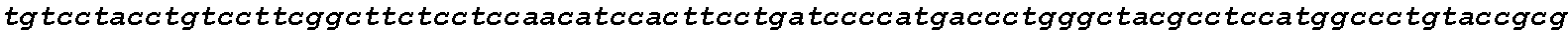

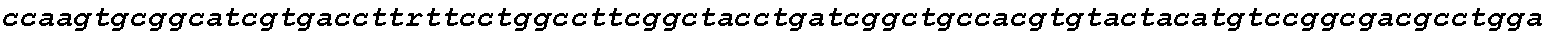

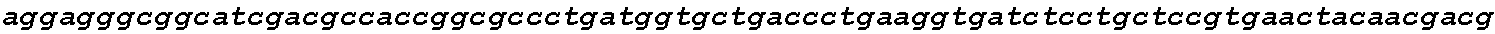

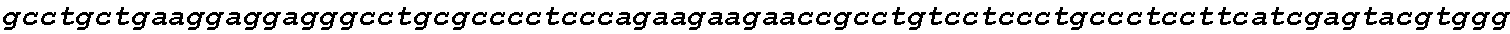

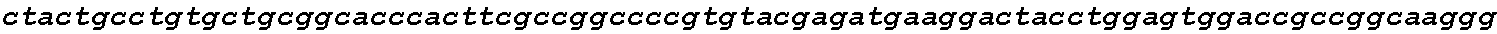

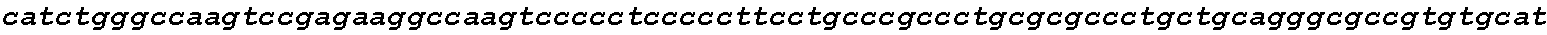

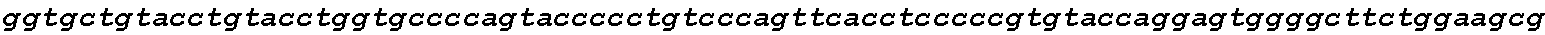

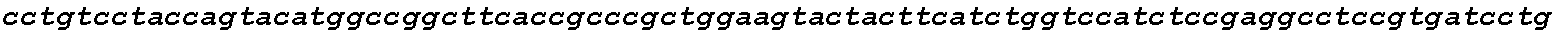

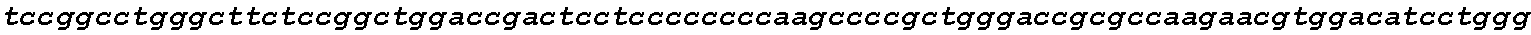

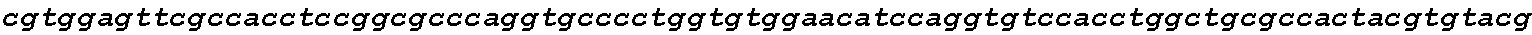

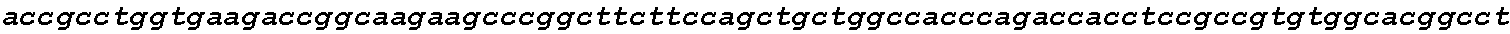

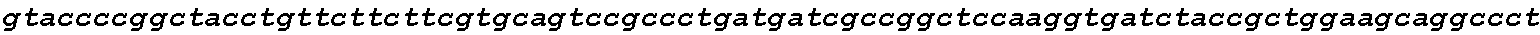

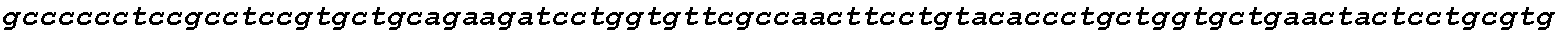

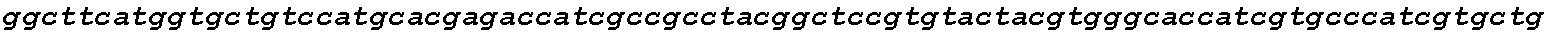

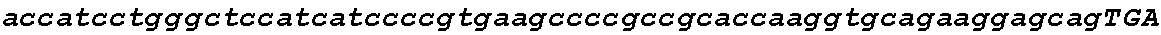

LimdLPCAT2
SEQ ID NO: 102

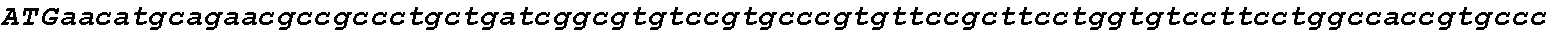

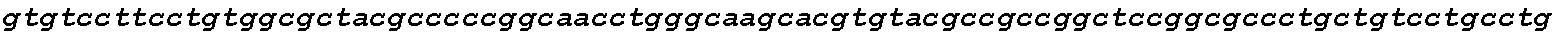

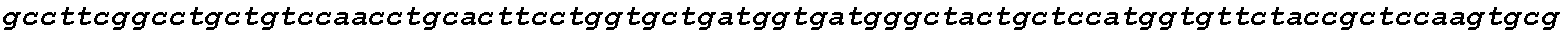

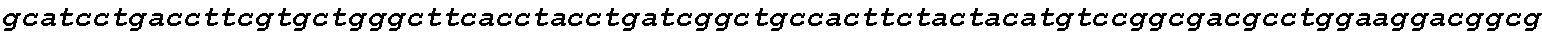

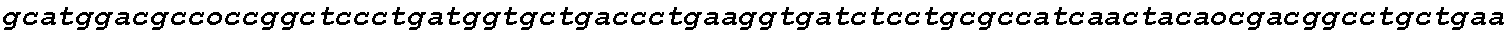

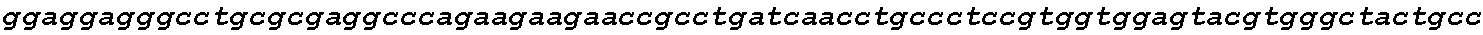

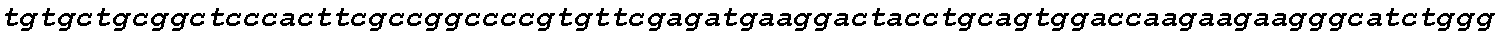

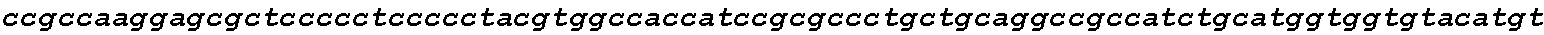

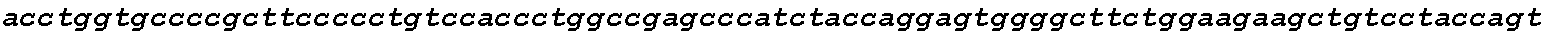

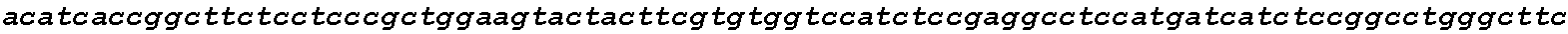

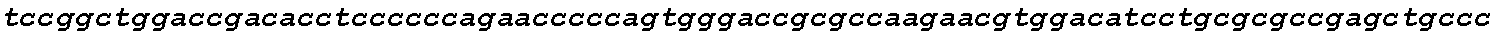

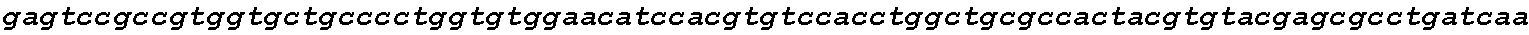

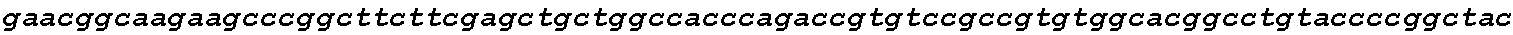

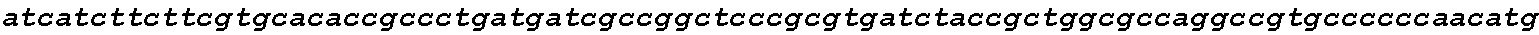

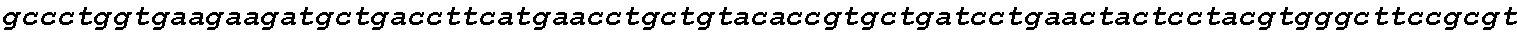

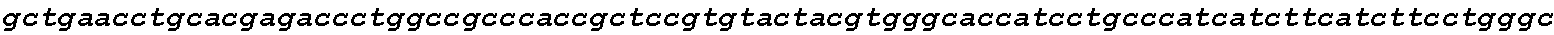

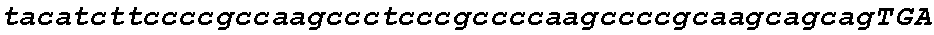

pSZ5297: AtLPCAT
SEQ ID NO: 103
gctcttctgcttcggattccactacatcaagtgggtgaacctggcgggcgcggaggagggcccccgcccgggcggcattgttagca
accactgcagctacctggacatcctgctgcacatgtccgattccttccccgcctttgtggcgcgccagtcgacggccaagctgccctt
tatcggcatcatcaggtgcgtgaaagtgggggctgctgtggtcgtggtgggcggggtcacaaatgaggacattgatgctgtcgttt
gccgatcaggggagctcgaaagtaagtgcagcctggtcatgggatcacaaatctcaccaccactcgtccaccttgcctgggccttg
cagccaaattatgagctgcctctacgtgaaccgcgaccgctcggggcccaaccacgtgggtgtggccgacctggtgaagcagcgc
atgcaggacgaggccgaggggaagaccccgcccgagtaccggccgctgctcctcttccccgaggtgggcttttgagacactgtttg
tgcttgaaactgtggacgcgcgtgccctgacgcgcctccggcgcctgtctcgcatccattcgcctctcaaccccatctcaccttttctc
catcgccagggcaccacctccaacggcgactacctgcttcccttcaagaccggcgccttcctggccggggtgcccgtccagcccgtg

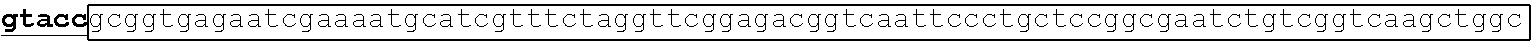

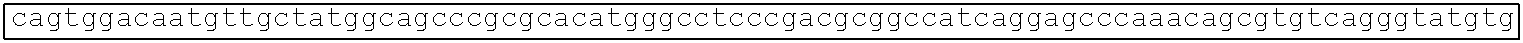

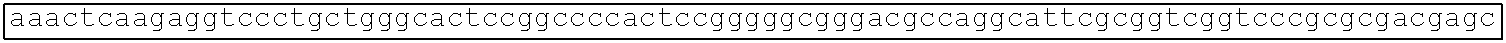

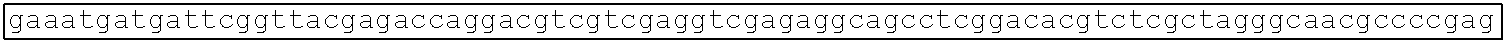

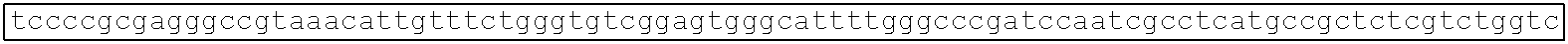

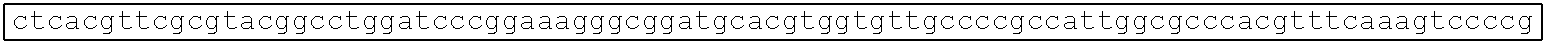

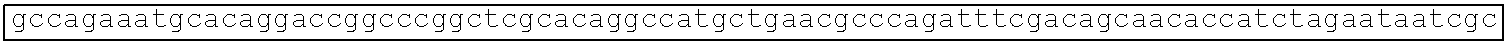

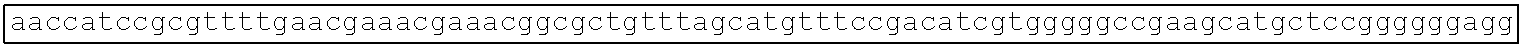

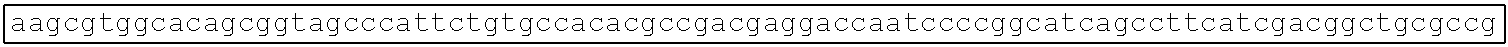

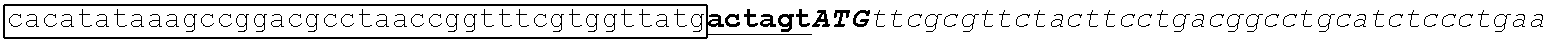

gggcgtgttcggcgtctccccctcctacaacggcctgggcctgacgccccagatgggctgggacaactggaacacgttcgcctg
cgacgtctccgagcagctgctgctggacacggccgaccgcatctccgacctgggcctgaaggacatgggctacaagtacatca
tcctggacgactgctggtcctccggccgcgactccgacggcttcctggtcgccgacgagcagaagttccccaacggcatgggcc
acgtcgccgaccacctgcacaacaactccttcctgttcggcatgtactcctccgcgggcgagtacacgtgcgccggctaccccgg
ctccctgggccgcgaggaggaggacgcccagttcttcgcgaacaaccgcgtggactacctgaagtacgacaactgctacaac
aagggccagttcggcacgcccgagatctcctaccaccgctacaaggccatgtccgacgccctgaacaagacgggccgcccca
tcttctactccctgtgcaactggggccaggacctgaccttctactggggctccggcatcgcgaactcctggcgcatgtccggcga
cgtcacggcggagttcacgcgccccgactcccgctgcccctgcgacggcgacgagtacgactgcaagtacgccggcttccact
gctccatcatgaacatcctgaacaaggccgcccccatgggccagaacgcgggcgtcggcggctggaacgacctggacaacct
ggaggtcggcgtcggcaacctgacggacgacgaggagaaggcgcacttctccatgtgggccatggtgaagtcccccctgatc
atcggcgcgaacgtgaacaacctgaaggcctcctcctactccatctactcccaggcgtccgtcatcgccatcaaccaggactcc
aacggcatccccgccacgcgcgtctggcgctactacgtgtccgacacggacgagtacggccagggcgagatccagatgtggt
ccggccccctggacaacggcgaccaggtcgtggcgctgctgaacggcggctccgtgtcccgccccatgaacacgaccctgga
ggagatcttcttcgactccaacctgggctccaagaagctgacctccacctgggacatctacgacctgtgggcgaaccgcgtcga
caactccacggcgtccgccatcctgggccgcaacaagaccgccaccggcatcctgtacaacgccaccgagcagtcctacaag
gacggcctgtccaagaacgacacccgcctgttcggccagaagatcggctccctgtcccccaacgcgatcctgaacacgaccgt

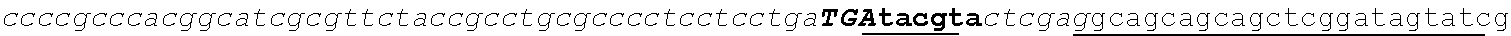

acacactctggacgctggtcgtgtgatggactgttgccgccacacttgctgccttgacctgtgaatatccctgccgcttttatcaaaca
gcctcagtgtgtttgatcttgtgtgtacgcgcttttgcgagttgctagctgcttgtgctatttgcgaataccacccccagcatccccttcc
ctcgtttcatatcgcttgcatcccaaccgcaacttatctacgctgtcctgctatccctcagcgctgctcctgctcctgctcactgcccctc
gcacagccttggtttgggctccgcctgtattctcctggtactgcaacctgtaaaccagcactgcaatgctgatgcacgggaagtagtg

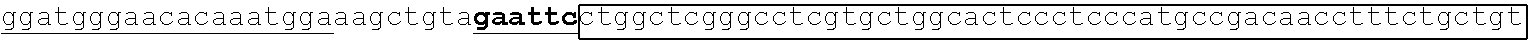

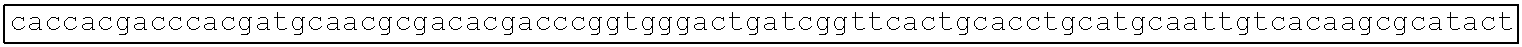

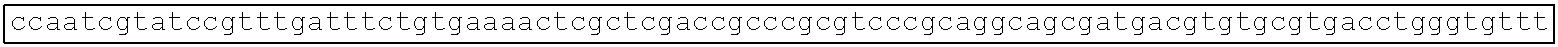

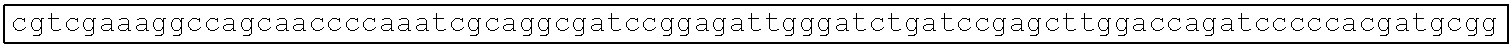

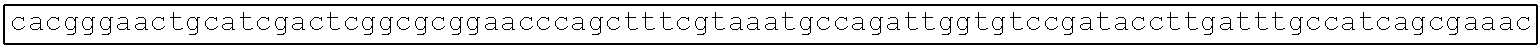

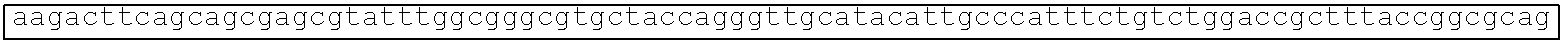

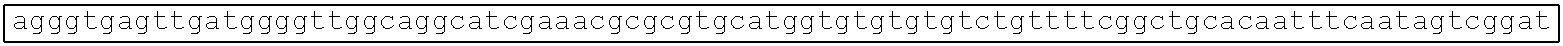

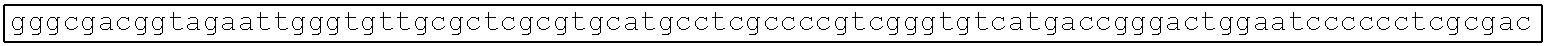

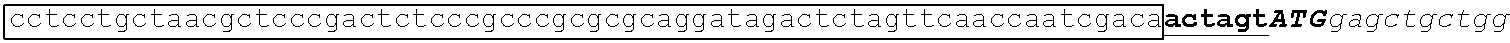

acatgaactccatggccgcctccatcggcgtgtccgtggccgtgctgcgcttcctgctgtgcttcgtggccaccatccccatctcct
tcctgtggcgcttcatcccctcccgcctgggcaagcacatctactccgccgcctccggcgccttcctgtcctacctgtccttcggcttc
tcctccaacctgcacttcctggtgcccatgaccatcggctacgcctccatggccatctaccgccccctgtccggcttcatcaccttct
tcctgggcttcgcctacctgatcggctgccacgtgttctacatgtccggcgacgcctggaaggagggcggcatcgactccaccg
gcgccctgatggtgctgaccctgaaggtgatctcctgctccatcaactacaacgacggcatgctgaaggaggagggcctgcgc
gaggcccagaagaagaaccgcctgatccagatgccctccctgatcgagtacttcggctactgcctgtgctgcggctcccacttc
gccggccccgtgttcgagatgaaggactacctggagtggaccgaggagaagggcatctgggccgtgtccgagaagggcaa
gcgcccctccccctacggcgccatgatccgcgccgtgttccaggccgccatctgcatggccctgtacctgtacctggtgccccagt
tccccctgacccgcttcaccgagcccgtgtaccaggagtggggcttcctgaagcgcttcggctaccagtacatggccggcttcac
cgcccgctggaagtactacttcatctggtccatctccgaggcctccatcatcatctccggcctgggcttctccggctggaccgacg
agacccagaccaaggccaagtgggaccgcgccaagaacgtggacatcctgggcgtggagctggccaagtccgccgtgcag
atccccctgttctggaacatccaggtgtccacctggctgcgccactacgtgtacgagcgcatcgtgaagcccggcaagaaggc
cggcttcttccagctgctggccacccagaccgtgtccgccgtgtggcacggcctgtaccccggctacatcatcttcttcgtgcagt
ccgccctgatgatcgacggctccaaggccatctaccgctggcagcaggccatcccccccaagatggccatgctgcgcaacgtg
ctggtgctgatcaacttcctgtacaccgtggtggtgctgaactactcctccgtgggcttcatggtgctgtccctgcacgagaccct
ggtggccttcaagtccgtgtactacatcggcaccgtgatccccatcgccgtgctgctgctgtcctacctggtgcccgtgaagcccg

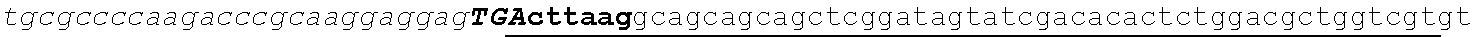

gatggactgttgccgccacacttgctgccttgacctgtgaatatccctgccgcttttatcaaacagcctcagtgtgtttgatcttgtgtgt
acgcgcttttgcgagttgctagctgcttgtgctatttgcgaataccacccccagcatccccttccctcgtttcatatcgcttgcatccca
accgcaacttatctacgctgtcctgctatccctcagcgctgctcctgctcctgctcactgcccctcgcacagccttggtttgggctccgc
ctgtattctcctggtactgcaacctgtaaaccagcactgcaatgctgatgcacgggaagtagtgggatgggaacacaaatggaaag
cttaattaagagctccgtcctccactaccacagggtatggtcgtgtggggtcgagcgtgttgaagcgcagaaggggatgcgccgtc
aagatcaggagctaaaaatggtgccagcgaggatccagcgctctcactcttgctgccatcgctcccacccttttccccaggggacc
ctgtggcccacgtgggagacgattccggccaagtggcacatcttcctgatgctctgccacccccgccacaaagtgaccgtgatgaa
ggttaggacaagggtcgggacccgattctggatatgacctctgaggtgtgtttctcgcgcaagcgtcccccaattcgttacaccaca
tccctcacaccctcgcccctgacactcgcagttgcccgtgtacgtccccaatgaggaggaaaaggccgaccccaagctgtacgccc
aaaacgtccgcaaagccatggtgcgtcgggaaccgtcaaagtttgcttgcgggtgggcggggcggctctagcgaattggctcatt
ggccctcaccgaggcagcacatcggacaccagtcgccacccggcttgcatcttcgccccctttcttctcgcagatggaggtcgccgg
gaccaaggacacgacggcggtgtttgaggacaagatgcgctacctgaactccctgaagagaaagtacggcaagcctgtgcctaa
gaaaattgagtgaacccccgtcgtcgaccagaagagc
pSZ5119
SEQ ID NO: 104
gctcttctgcttcggattccactacatcaagtgggtgaacctggcgggcgcggaggagggcccccgcccgggcggcattgttagca
accactgcagctacctggacatcctgctgcacatgtccgattccttccccgcctttgtggcgcgccagtcgacggccaagctgccctt
tatcggcatcatcaggtgcgtgaaagtgggggctgctgtggtcgtggtgggcggggtcacaaatgaggacattgatgctgtcgttt
gccgatcaggggagctcgaaagtaagtgcagcctggtcatgggatcacaaatctcaccaccactcgtccaccttgcctgggccttg
cagccaaattatgagctgcctctacgtgaaccgcgaccgctcggggcccaaccacgtgggtgtggccgacctggtgaagcagcgc
atgcaggacgaggccgaggggaagaccccgcccgagtaccggccgctgctcctcttccccgaggtgggcttttgagacactgtttg
tgcttgaaactgtggacgcgcgtgccctgacgcgcctccggcgcctgtctcgcatccattcgcctctcaaccccatctcaccttttctc
catcgccagggcaccacctccaacggcgactacctgcttcccttcaagaccggcgccttcctggccggggtgcccgtccagcccgtg

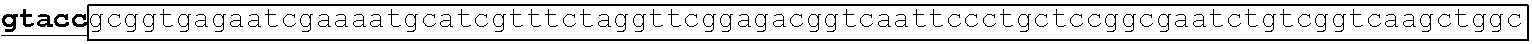

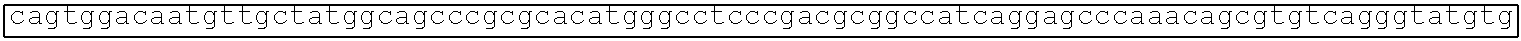

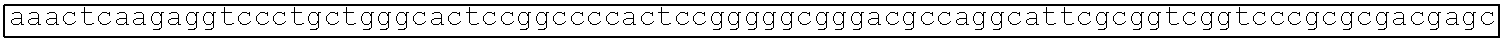

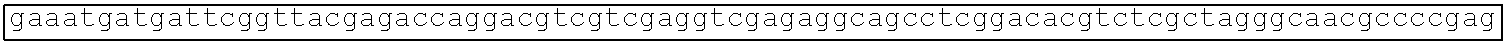

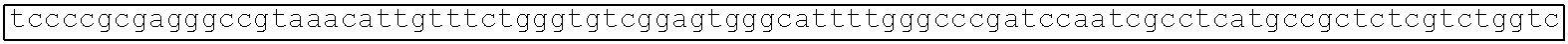

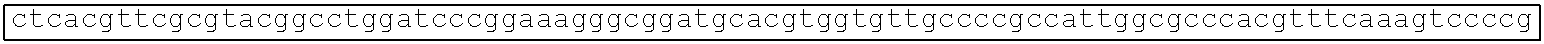

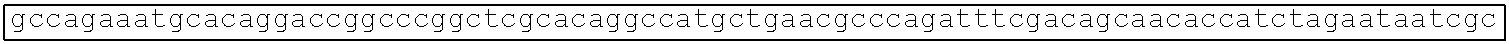

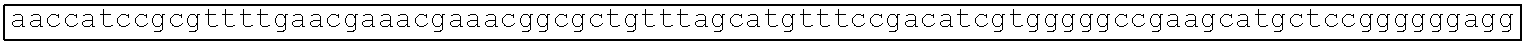

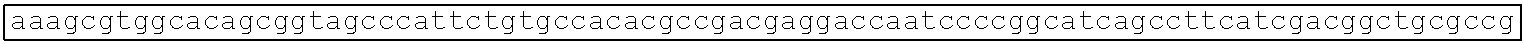

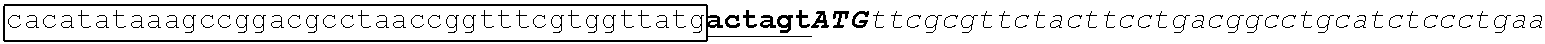

gggcgtgttcggcgtctccccctcctacaacggcctgggcctgacgccccagatgggctgggacaactggaacacgttcgcctg
cgacgtctccgagcagctgctgctggacacggccgaccgcatctccgacctgggcctgaaggacatgggctacaagtacatca
tcctggacgactgctggtcctccggccgcgactccgacggcttcctggtcgccgacgagcagaagttccccaacggcatgggcc
acgtcgccgaccacctgcacaacaactccttcctgttcggcatgtactcctccgcgggcgagtacacgtgcgccggctaccccgg
ctccctgggccgcgaggaggaggacgcccagttcttcgcgaacaaccgcgtggactacctgaagtacgacaactgctacaac
aagggccagttcggcacgcccgagatctcctaccaccgctacaaggccatgtccgacgccctgaacaagacgggccgcccca
tcttctactccctgtgcaactggggccaggacctgaccttctactggggctccggcatcgcgaactcctggcgcatgtccggcga
cgtcacggcggagttcacgcgccccgactcccgctgcccctgcgacggcgacgagtacgactgcaagtacgccggcttccact
gctccatcatgaacatcctgaacaaggccgcccccatgggccagaacgcgggcgtcggcggctggaacgacctggacaacct
ggaggtcggcgtcggcaacctgacggacgacgaggagaaggcgcacttctccatgtgggccatggtgaagtcccccctgatc
atcggcgcgaacgtgaacaacctgaaggcctcctcctactccatctactcccaggcgtccgtcatcgccatcaaccaggactcc
aacggcatccccgccacgcgcgtctggcgctactacgtgtccgacacggacgagtacggccagggcgagatccagatgtggt
ccggccccctggacaacggcgaccaggtcgtggcgctgctgaacggcggctccgtgtcccgccccatgaacacgaccctgga
ggagatcttcttcgactccaacctgggctccaagaagctgacctccacctgggacatctacgacctgtgggcgaaccgcgtcga
caactccacggcgtccgccatcctgggccgcaacaagaccgccaccggcatcctgtacaacgccaccgagcagtcctacaag
gacggcctgtccaagaacgacacccgcctgttcggccagaagatcggctccctgtcccccaacgcgatcctgaacacgaccgt

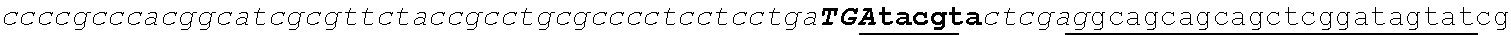

acacactctggacgctggtcgtgtgatggactgttgccgccacacttgctgccttgacctgtgaatatccctgccgcttttatcaaaca
gcctcagtgtgtttgatcttgtgtgtacgcgcttttgcgagttgctagctgcttgtgctatttgcgaataccacccccagcatccccttcc
ctcgtttcatatcgcttgcatcccaaccgcaacttatctacgctgtcctgctatccctcagcgctgctcctgctcctgctcactgcccctc
gcacagccttggtttgggctccgcctgtattctcctggtactgcaacctgtaaaccagcactgcaatgctgatgcacgggaagtagtg

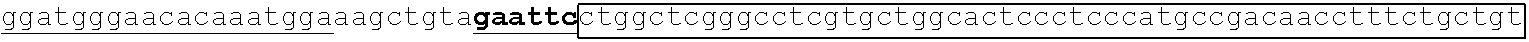

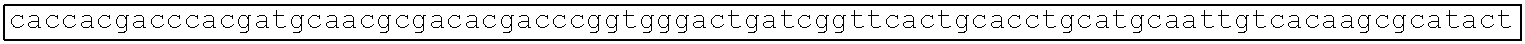

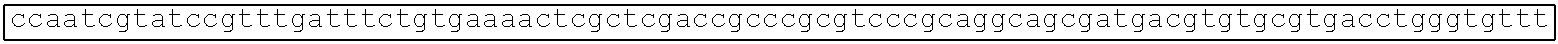

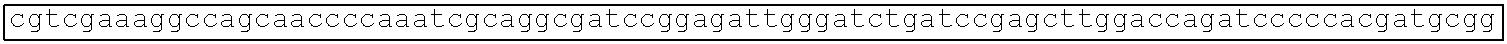

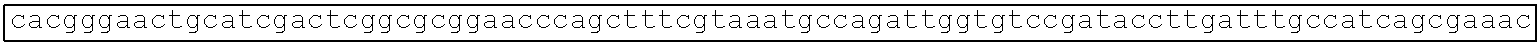

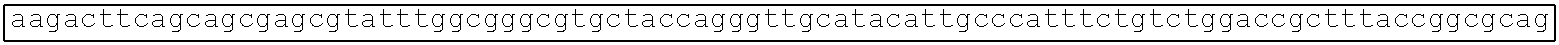

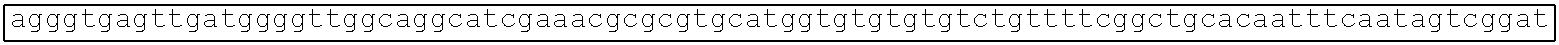

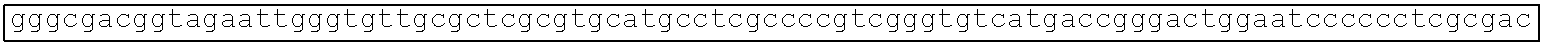

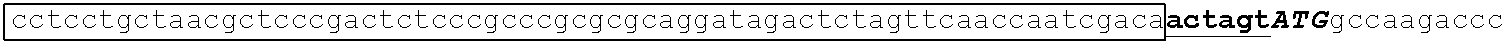

gcacctcctccctgcgcaaccgccgccagctgaagcccgccgtggccgccaccgccgacgacgacaaggacggcgtgttcatg
gtgctgctgtcctgcttcaagatcttcgtgtgcttcgccatcgtgctgatcaccgccgtggcctggggcctgatcatggtgctgctg
ctgccctggccctccctgcgcctccgcctgggccccctgtccggccccctcatcggcggcctggtgctctggctctacggcatcc
ccatcacgatcccgggctccgcgccccccccgcagcgcgccatctacatctccaaccacgcctcccccatcgccgccttcttcgt
gatgtggctggcccccctcggccccgtgggcgtggccaagacggcggtgatctggtaccccctgctgggcccgctgtcccccc
tggcccaccacatccgcatcgaccgctccaaccccgccgccgccatccagtccatgaaggaggccgtgcgcgtgatcaccgag
aagaacctgtccctgatcatgttccccgagggcacccgctcccgcgacggccgcctgctgcccttcccgccgggcttcgtgccc
ctggccctgcagtcccacctgcccatcgtgcccatgatcctgaccggcacccacctggcctggcgcaagggcaccttccgcgtgc
gccccgtgcccatcaccgtgaagtacctgccccccatcaacaccgacgactggaccgtggacaagatcgacgactacgtgaa

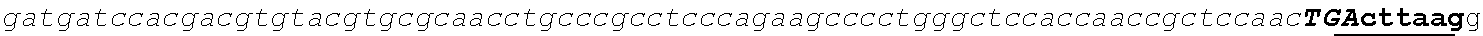

cagcagcagctcggatagtatcgacacactctggacgctggtcgtgtgatggactgttgccgccacacttgctgccttgacctgtgaa
tatccctgccgcttttatcaaacagcctcagtgtgtttgatcttgtgtgtacgcgcttttgcgagttgctagctgcttgtgctatttgcga
ataccacccccagcatccccttccctcgtttcatatcgcttgcatcccaaccgcaacttatctacgctgtcctgctatccctcagcgctg
ctcctgctcctgctcactgcccctcgcacagccttggtttgggctccgcctgtattctcctggtactgcaacctgtaaaccagcactgca
atgctgatgcacgggaagtagtgggatgggaacacaaatggaaagcttaattaagagctccgtcctccactaccacagggtatggt
cgtgtggggtcgagcgtgttgaagcgcagaaggggatgcgccgtcaagatcaggagctaaaaatggtgccagcgaggatccag
cgctctcactcttgctgccatcgctcccaccdtttccccaggggaccctgtggcccacgtgggagacgattccggccaagtggcac
atcttcctgatgctctgccacccccgccacaaagtgaccgtgatgaaggttaggacaagggtcgggacccgattctggatatgacc
tctgaggtgtgtttctcgcgcaagcgtcccccaattcgttacaccacatccctcacaccctcgcccctgacactcgcagttgcccgtg
tacgtccccaatgaggaggaaaaggccgaccccaagctgtacgcccaaaacgtccgcaaagccatggtgcgtcgggaaccgtca
aagtttgcttgcgggtgggcggggcggctctagcgaattggctcattggccctcaccgaggcagcacatcggacaccagtcgccac
ccggcttgcatcttcgccccctttcttctcgcagatggaggtcgccgggaccaaggacacgacggcggtgtttgaggacaagatgc
gctacctgaactccctgaagagaaagtacggcaagcctgtgcctaagaaaattgagtgaacccccgtcgtcgaccagaagagc
Sequence of PLSC-2/LPAAT1-2 5′ flank in pSZ5120 and pSZ5348
SEQ ID NO: 105
gctcttctgcttcggattccactacatcaagtgggtgaacctggcgggcgcggaggagggcccccgcccgggcggcat
tgttagcaaccactgcagctacctggacatcctgctgcacatgtccgactccttccccgcctttgtggcgcgccagtcga
cggccaagctgccctttatcggcatcatcaggtgcgtgaaagcgggggctgctgtggccgtggtgggcagggttgcga
aggggggcaggcgtaggcgtgcagtgtgagcggacattgatgccgtcgtttgccggtcaggagagctcgaaatcaga
gccagcctggtcatgggatcacagagctcaccaccactcgtccacctcgcctgcgccttgcagccaaatcatgagctgc
ctctacgtgaaccgcgaccgctcggggcccaaccacgtgggcgtggccgatctggtgaagcagcgcatgcaggacga
ggccgaggggaggaccccgcccgagtaccgaccgctgctcctcttccccgaggtgggctttcgaggcaccgtttgtgct
tgaaactgtgggcacgcgtgccccgacgcgcctctggcgcctgcttcgcatccattcgcctctcaaccccgtctctccttt
cctccatcgccagggcaccacctccaacggcgactacctgcttcccttcaagaccggcgccttcctggccggggtgccc
gtccagcccgtggtacc
PLSC-2/LPAAT1-2 3′ flank in pSZ5120 and pSZ5348
SEQ ID NO: 106
gagctccgtcctccactaccacagggtatggtggtgtggggtcgagcgtgttgaagcgcggaaggggatgcgctgtca
agttttggagctgaaaatggtgcccgcgaggatccagcgcgccccactcacccttgctgccatcgctccccacccttttc
cccagggaaccctgtggcccacgtgggagacgattccggccaagtggcacatcttcctgatgctctgccacccccgcc
acaaagtgaccgtgatgaaggtacgaacaagggtcgggccccgattctggatatcacgtctggggtgtgtttctcgcg
cacgcgtcccccgatgcgctgcacagtctccctcacaccctcacccctaacgctcgcagttgcccgtgtacgtccccaat
gaggaggaaaaggccgaccccaagctgtacgcccaaaatgttcgcaaagccatggtgcgtcgggaaccgttcaagtt
tgcttgcgggtgggcggggcggctctagcgaattggcgcattggccctcaccgaggcagcacatcggacaccaatcgt
cacccggcgagcaattccgccccctctgtcttctcgcagatggaggtcgccgggaccaaggacacgacggcggtgttt
gaggacaagatgcgctacctgaactccctgaagagaaagtacggcaagcctgtgcctaagaaaattgagtgaacccc
cgtcgtcgaccagaagagc
L. alba LPAAT (LimaLPAAT) contained in pSZ5343 and pSZ5348
SEQ ID NO: 107

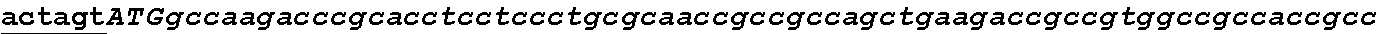

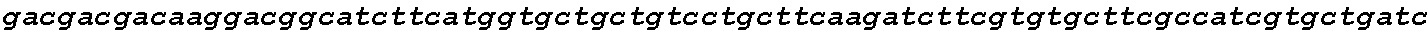

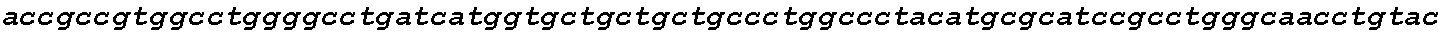

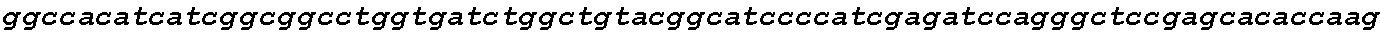

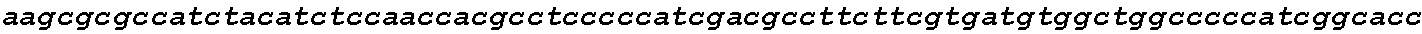

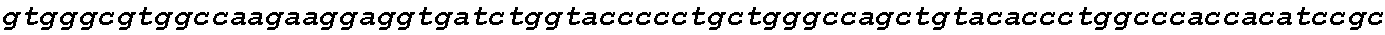

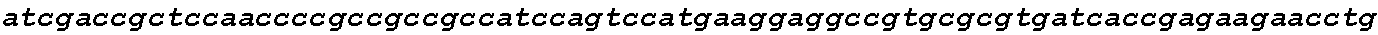

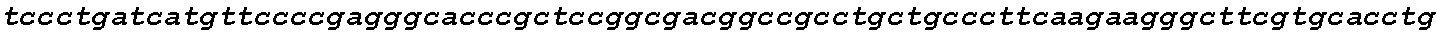

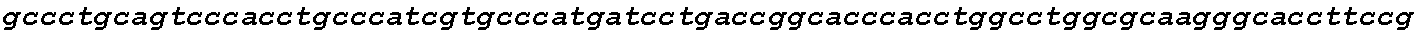

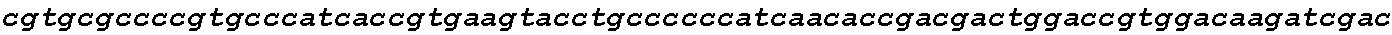

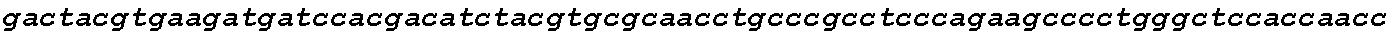

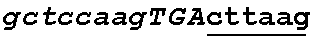

B. Juncea LPCAT1 (BjLPCAT1) contained in pSZ5346 and pSZ5351
SEQ ID NO: 108

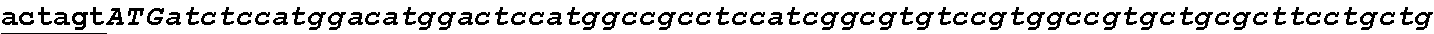

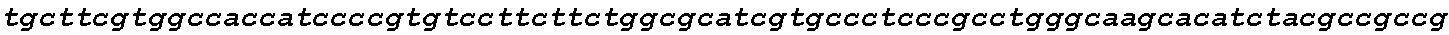

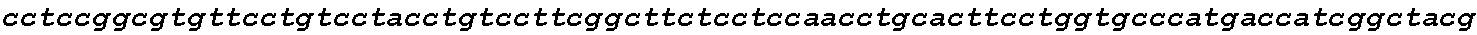

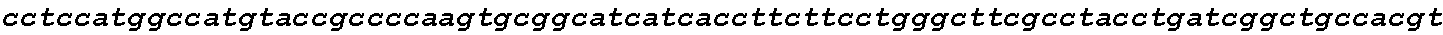

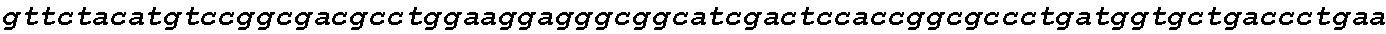

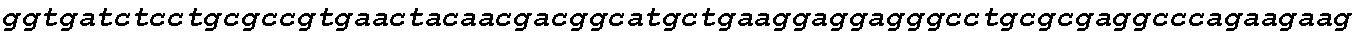

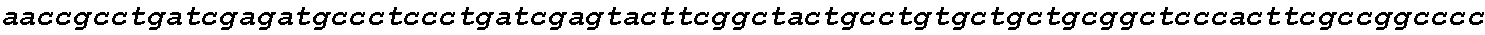

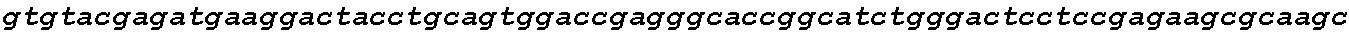

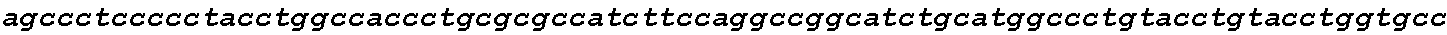

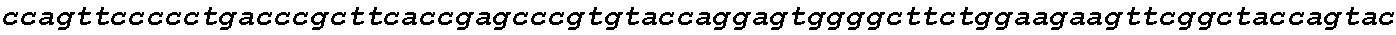

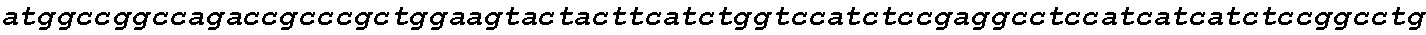

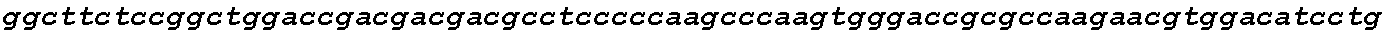

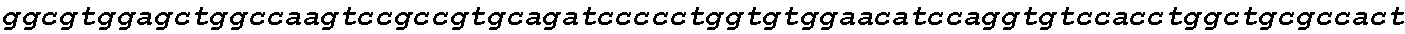

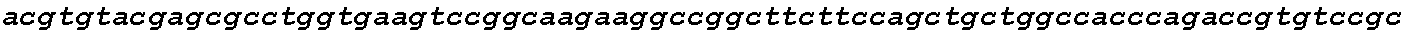

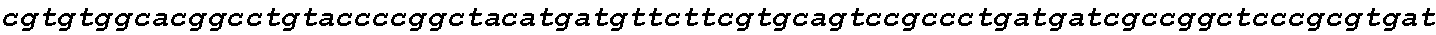

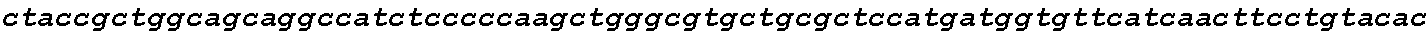

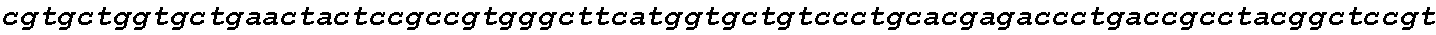

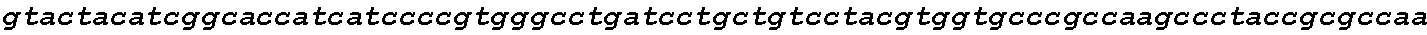

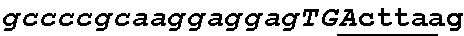

B. juncea LPCAT2 (BjLPCAT2) contained in pSZ5298 and pSZ5352
SEQ ID NO: 109

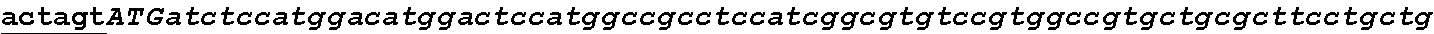

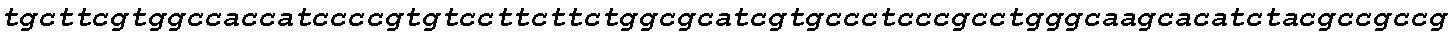

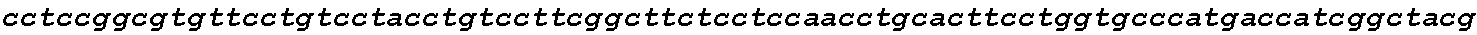

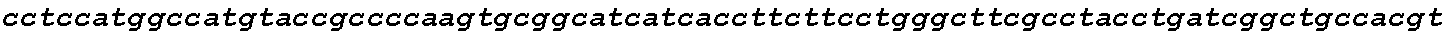

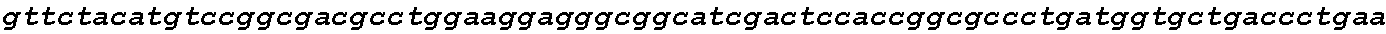

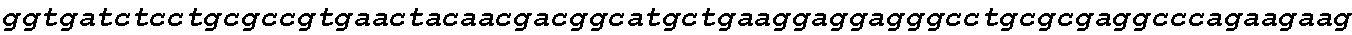

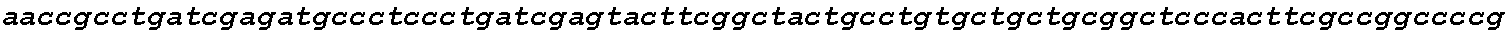

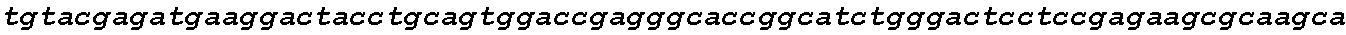

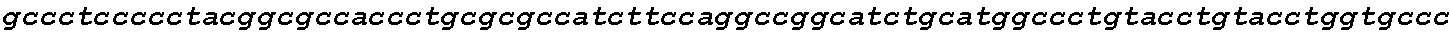

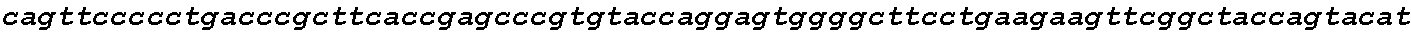

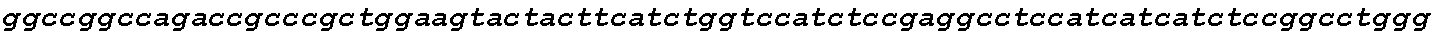

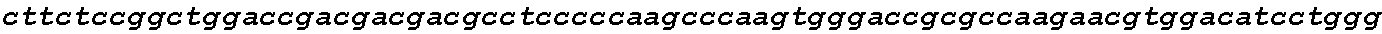

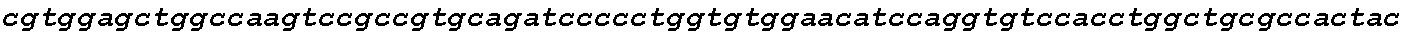

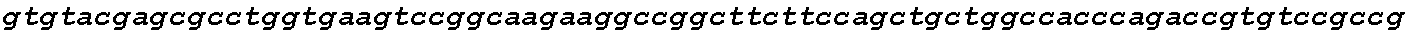

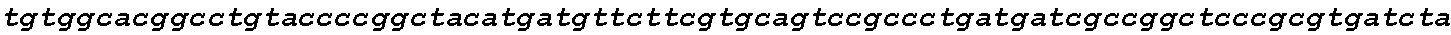

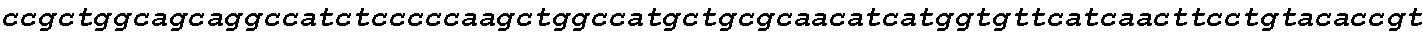

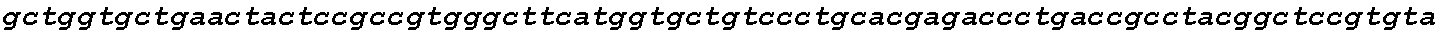

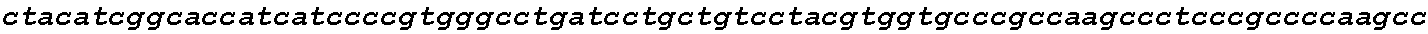

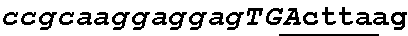

PSZ5298
SEQ ID NO: 110
gctcttctgcttcggattccactacatcaagtgggtgaacctggcgggcgcggaggagggcccccgcccgggcggcattgttagca
accactgcagctacctggacatcctgctgcacatgtccgattccttccccgcctttgtggcgcgccagtcgacggccaagctgccctt
tatcggcatcatcaggtgcgtgaaagtgggggctgctgtggtcgtggtgggcggggtcacaaatgaggacattgatgctgtcgttt
gccgatcaggggagctcgaaagtaagtgcagcctggtcatgggatcacaaatctcaccaccactcgtccaccttgcctgggccttg
cagccaaattatgagctgcctctacgtgaaccgcgaccgctcggggcccaaccacgtgggtgtggccgacctggtgaagcagcgc
atgcaggacgaggccgaggggaagaccccgcccgagtaccggccgctgctcctcttccccgaggtgggcttttgagacactgtttg
tgcttgaaactgtggacgcgcgtgccctgacgcgcctccggcgcctgtctcgcatccattcgcctctcaaccccatctcaccttttctc
catcgccagggcaccacctccaacggcgactacctgcttcccttcaagaccggcgccttcctggccggggtgcccgtccagcccgtg

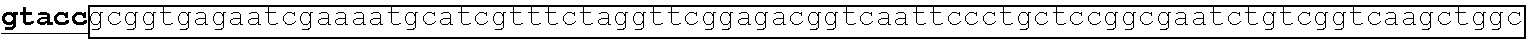

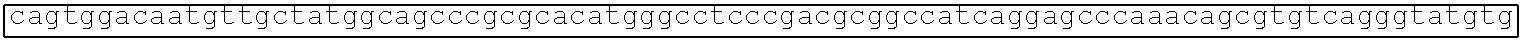

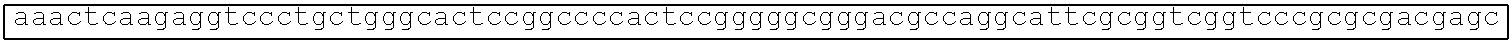

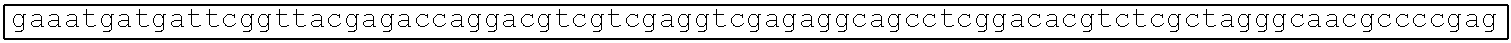

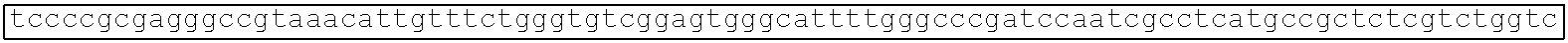

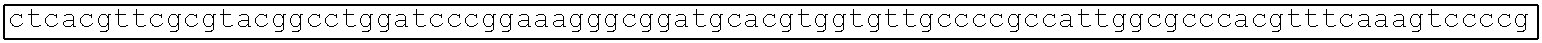

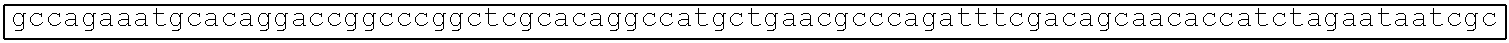

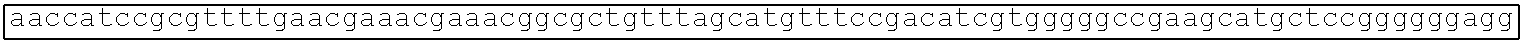

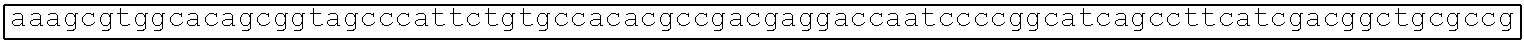

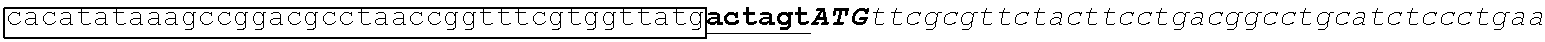

gggcgtgttcggcgtctccccctcctacaacggcctgggcctgacgccccagatgggctgggacaactggaacacgttcgcctg
cgacgtctccgagcagctgctgctggacacggccgaccgcatctccgacctgggcctgaaggacatgggctacaagtacatca
tcctggacgactgctggtcctccggccgcgactccgacggcttcctggtcgccgacgagcagaagttccccaacggcatgggcc
acgtcgccgaccacctgcacaacaactccttcctgttcggcatgtactcctccgcgggcgagtacacgtgcgccggctaccccgg
ctccctgggccgcgaggaggaggacgcccagttcttcgcgaacaaccgcgtggactacctgaagtacgacaactgctacaac
aagggccagttcggcacgcccgagatctcctaccaccgctacaaggccatgtccgacgccctgaacaagacgggccgcccca
tcttctactccctgtgcaactggggccaggacctgaccttctactggggctccggcatcgcgaactcctggcgcatgtccggcga
cgtcacggcggagttcacgcgccccgactcccgctgcccctgcgacggcgacgagtacgactgcaagtacgccggcttccact
gctccatcatgaacatcctgaacaaggccgcccccatgggccagaacgcgggcgtcggcggctggaacgacctggacaacct
ggaggtcggcgtcggcaacctgacggacgacgaggagaaggcgcacttctccatgtgggccatggtgaagtcccccctgatc
atcggcgcgaacgtgaacaacctgaaggcctcctcctactccatctactcccaggcgtccgtcatcgccatcaaccaggactcc
aacggcatccccgccacgcgcgtctggcgctactacgtgtccgacacggacgagtacggccagggcgagatccagatgtggt
ccggccccctggacaacggcgaccaggtcgtggcgctgctgaacggcggctccgtgtcccgccccatgaacacgaccctgga
ggagatcttcttcgactccaacctgggctccaagaagctgacctccacctgggacatctacgacctgtgggcgaaccgcg
caactccacggcgtccgccatcctgggccgcaacaagaccgccaccggcatcctgtacaacgccaccgagcagtcctacaag
gacggcctgtccaagaacgacacccgcctgttcggccagaagatcggctccctgtcccccaacgcgatcctgaacacgaccgt

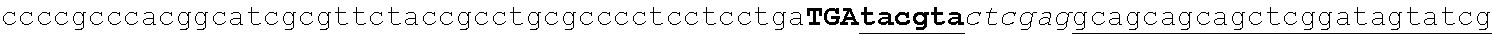

acacactctggacgctggtcgtgtgatggactgttgccgccacacttgctgccttgacctgtgaatatccctgccgcttttatcaaaca
gcctcagtgtgtttgatcttgtgtgtacgcgcttttgcgagttgctagctgcttgtgctatttgcgaataccacccccagcatccccttcc
ctcgtttcatatcgcttgcatcccaaccgcaacttatctacgctgtcctgctatccctcagcgctgctcctgctcctgctcactgcccctc
gcacagccttggtttgggctccgcctgtattctcctggtactgcaacctgtaaaccagcactgcaatgctgatgcacgggaagtagtg

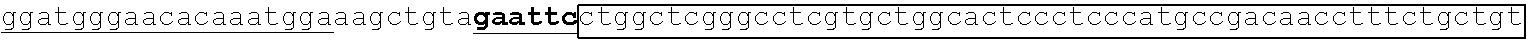

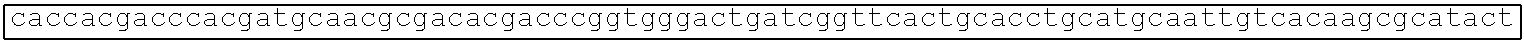

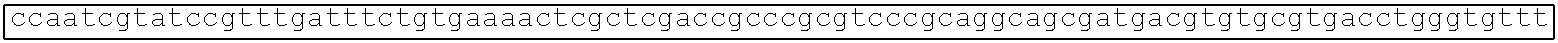

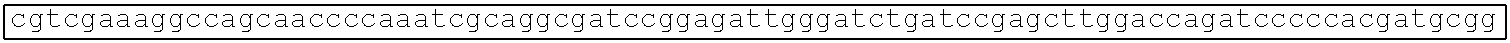

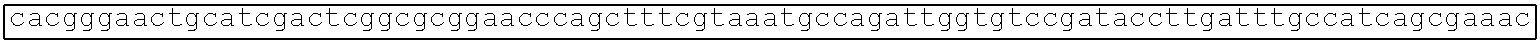

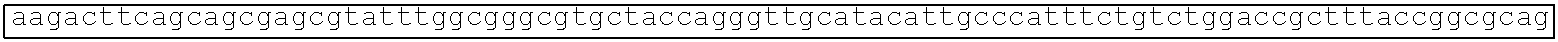

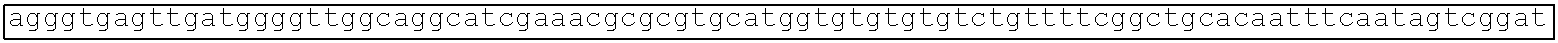

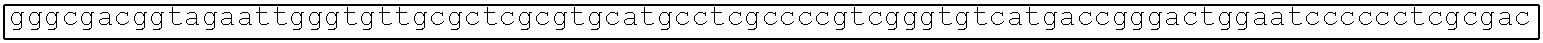

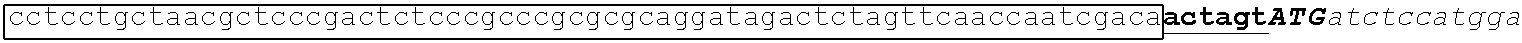

catgaactccatggccgcctccatcggcgtgtccgtggccgtgctgcgcttcctgctgtgcttcgtggccaccatccccgtgtcctt
cgcctggcgcatcgtgccctcccgcctgggcaagcacatctacgccgccgcctccggcgtgttcctgtcctacctgtccttcggctt
ctcctccaacctgcacttcctggtgcccatgaccatcggctacgcctccatggccatgtaccgccccaagtgcggcatcatcacct
tcttcctgggcttcgcctacctgatcggctgccacgtgttctacatgtccggcgacgcctggaaggagggcggcatcgactccac
cggcgccctgatggtgctgaccctgaaggtgatctcctgcgccgtgaactacaacgacggcatgctgaaggaggagggcctg
cgcgaggcccagaagaagaaccgcctgatccagatgccctccctgatcgagtacttcggctactgcctgtgctgcggctcccac
ttcgccggccccgtgtacgagatgaaggactacctgcagtggaccgagggcaagggcatctgggactcctccgagaagcgc
aagcagccctccccctacggcgccaccctgcgcgccatcttccaggccggcatctgcatggccctgtacctgtacctggtgcccc
agttccccctgacccgcttcaccgagcccgtgtaccaggagtggggcttcctgaagaagttcggctaccagtacatggccggcc
agaccgcccgctggaagtactacttcatctggtccatctccgaggcctccatcatcatctccggcctgggcttctccggctggacc
gacgacgacgcctcccccaagcccaagtgggaccgcgccaagaacgtggacatcctgggcgtggagctggccaagtccgcc
gtgcagatccccctggtgtggaacatccaggtgtccacctggctgcgccactacgtgtacgagcgcctggtgaagtccggcaa
gaaggccggcttcttccagctgctggccacccagaccgtgtccgccgtgtggcacggcctgtaccccggctacatgatgttcttc
gtgcagtccgccctgatgatcgccggctcccgcgtgatctaccgctggcagcaggccatctcccccaagctggccatgctgcgc
aacatcatggtgttcatcaacttcctgtacaccgtgctggtgctgaactactccgccgtgggcttcatggtgctgtccctgcacga
gaccctgaccgcctacggctccgtgtactacatcggcaccatcatccccgtgggcctgatcctgctgtcctacgtggtgcccgcca

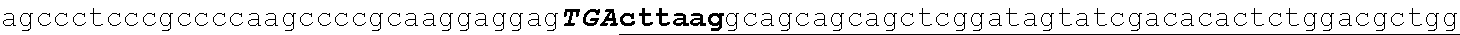

tcgtgtgatggactgttgccgccacacttgctgccttgacctgtgaatatccctgccgcttttatcaaacagcctcagtgtgtttgatctt
gtgtgtacgcgcttttgcgagttgctagctgcttgtgctatttgcgaataccacccccagcatccccttccctcgtttcatatcgcttgca
tcccaaccgcaacttatctacgctgtcctgctatccctcagcgctgctcctgctcctgctcactgcccctcgcacagccttggtttgggc
tccgcctgtattctcctggtactgcaacctgtaaaccagcactgcaatgctgatgcacgggaagtagtgggatgggaacacaaatgg
aaagcttaattaagagctccgtcctccactaccacagggtatggtcgtgtggggtcgagcgtgttgaagcgcagaaggggatgcg
ccgtcaagatcaggagctaaaaatggtgccagcgaggatccagcgctctcactcttgctgccatcgctcccacccttttccccaggg
gaccctgtggcccacgtgggagacgattccggccaagtggcacatcttcctgatgctctgccacccccgccacaaagtgaccgtga
tgaaggttaggacaagggtcgggacccgattctggatatgacctctgaggtgtgtttctcgcgcaagcgtcccccaattcgttacac
cacatccctcacaccctcgcccctgacactcgcagttgcccgtgtacgtccccaatgaggaggaaaaggccgaccccaagctgtac
gcccaaaacgtccgcaaagccatggtgcgtcgggaaccgtcaaagtttgcttgcgggtgggcggggcggctctagcgaattggct
cattggccctcaccgaggcagcacatcggacaccagtcgccacccggcttgcatcttcgccccctttcttctcgcagatggaggtcg
ccgggaccaaggacacgacggcggtgtttgaggacaagatgcgctacctgaactccctgaagagaaagtacggcaagcctgtgc
ctaagaaaattgagtgaacccccgtcgtcgaccagaagagc
SEQ ID NO: 111
gctcttctgcttcggattccactacatcaagtgggtgaacctggcgggcgcggaggagggcccccgcccgggcggcattgtta
gcaaccactgcagctacctggacatcctgctgcacatgtccgactccttccccgcctttgtggcgcgccagtcgacggccaagc
tgccctttatcggcatcatcaggtgcgtgaaagcgggggctgctgtggccgtggtgggcagggttgcgaaggggggcaggcg
taggcgtgcagtgtgagcggacattgatgccgtcgtttgccggtcaggagagctcgaaatcagagccagcctggtcatgggat
cacagagctcaccaccactcgtccacctcgcctgcgccttgcagccaaatcatgagctgcctctacgtgaaccgcgaccgctc
ggggcccaaccacgtgggcgtggccgatctggtgaagcagcgcatgcaggacgaggccgaggggaggaccccgcccgagt
accgaccgctgctcctcttccccgaggtgggctttcgaggcaccgtttgtgcttgaaactgtgggcacgcgtgccccgacgcgc
ctctggcgcctgcttcgcatccattcgcctctcaaccccgtctctcctttcctccatcgccagggcaccacctccaacggcgacta
cctgcttcccttcaagaccggcgccttcctggccggggtgcccgtccagcccgtggtacc
SEQ ID NO: 112
gagctccgtcctccactaccacagggtatggtggtgtggggtcgagcgtgttgaagcgcggaaggggatgcgctgtcaagttt
tggagctgaaaatggtgcccgcgaggatccagcgcgccccactcacccttgctgccatcgctccccacccttttccccagggaa
ccctgtggcccacgtgggagacgattccggccaagtggcacatcttcctgatgctctgccacccccgccacaaagtgaccgtg
atgaaggtacgaacaagggtcgggccccgattctggatatcacgtctggggtgtgtttctcgcgcacgcgtcccccgatgcgct
gcacagtctccctcacaccctcacccctaacgctcgcagttgcccgtgtacgtccccaatgaggaggaaaaggccgaccccaa
gctgtacgcccaaaatgttcgcaaagccatggtgcgtcgggaaccgttcaagtttgcttgcgggtgggcggggcggctctagc
gaattggcgcattggccctcaccgaggcagcacatcggacaccaatcgtcacccggcgagcaattccgccccctctgtcttctc
gcagatggaggtcgccgggaccaaggacacgacggcggtgtttgaggacaagatgcgctacctgaactccctgaagagaaa
gtacggcaagcctgtgcctaagaaaattgagtgaacccccgtcgtcgaccagaagagc
SEQ ID NO: 113

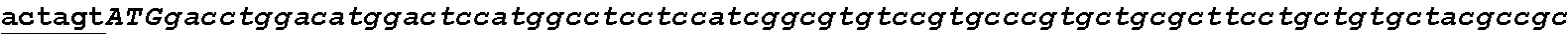

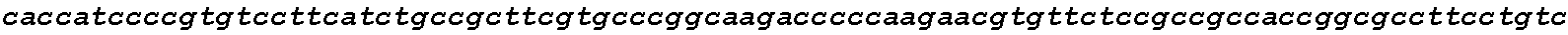

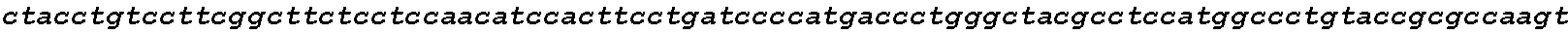

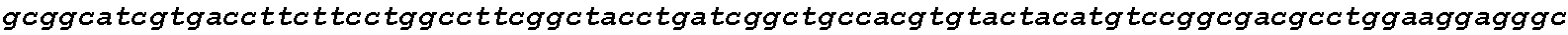

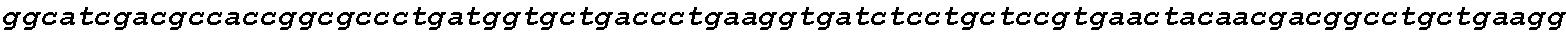

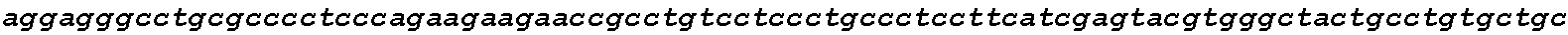

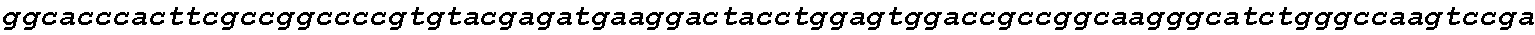

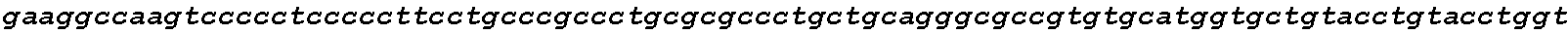

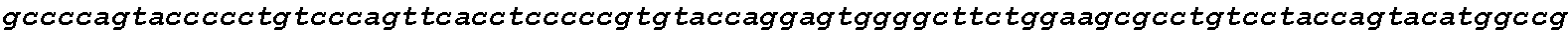

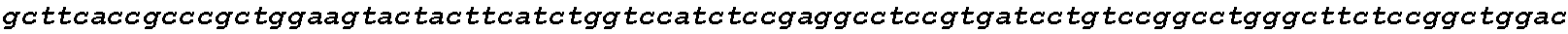

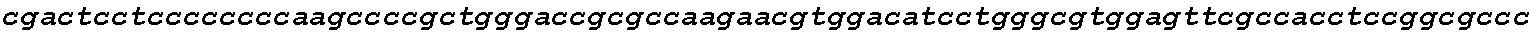

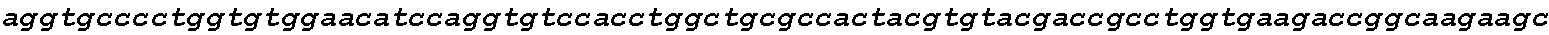

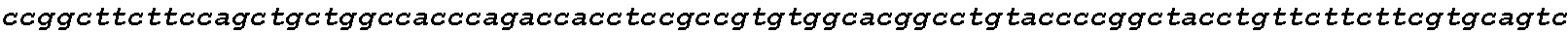

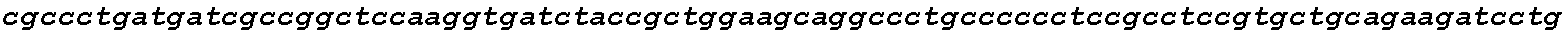

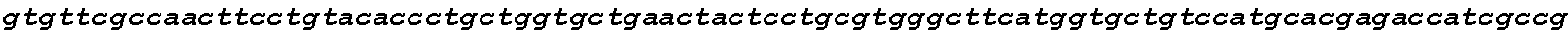

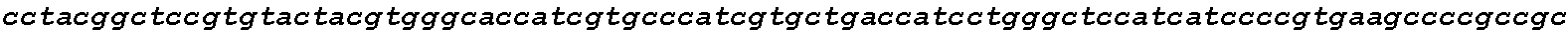

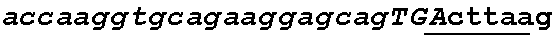

SEQ ID NO: 114

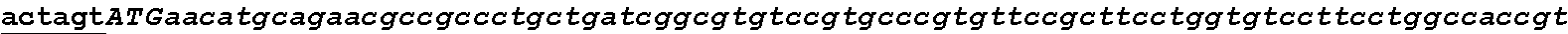

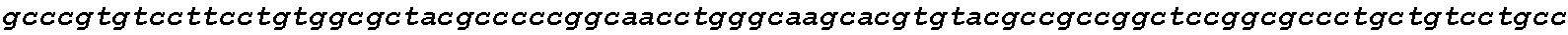

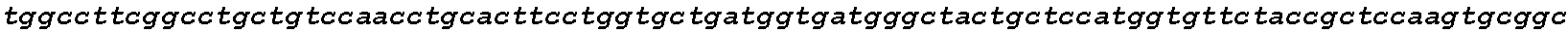

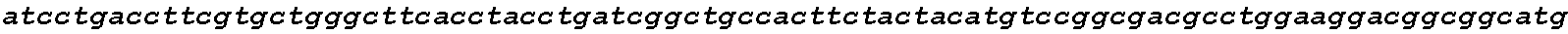

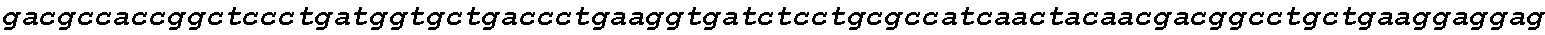

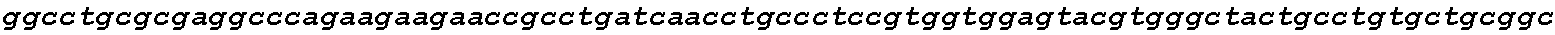

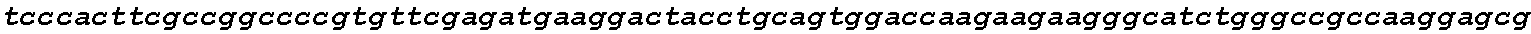

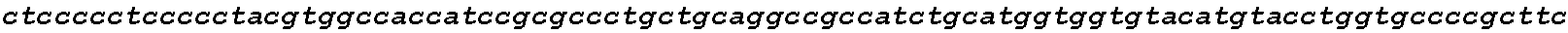

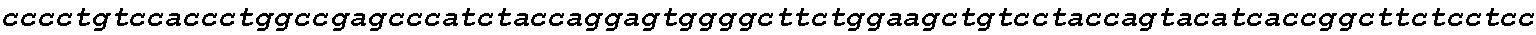

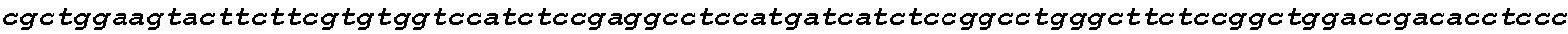

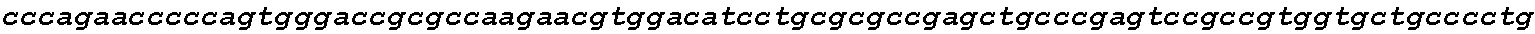

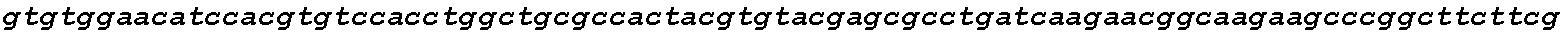

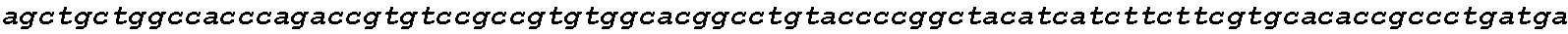

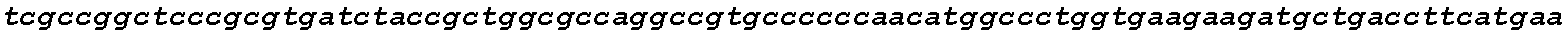

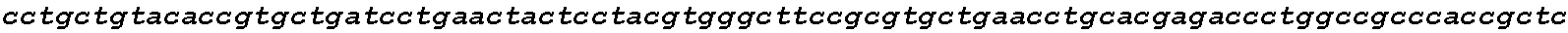

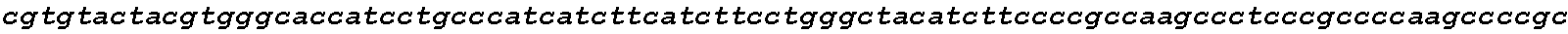

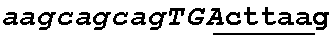

SEQ ID NO: 115

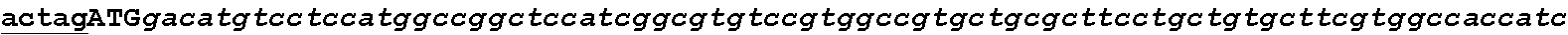

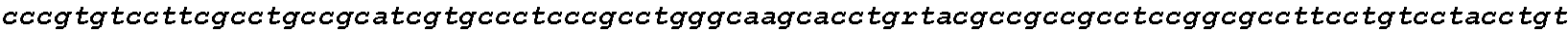

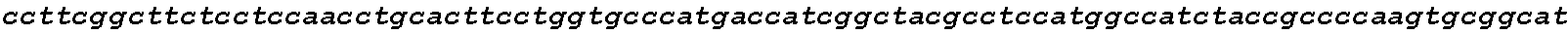

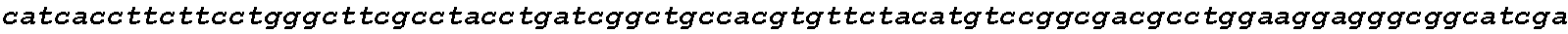

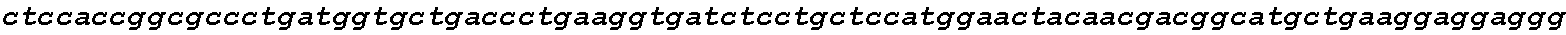

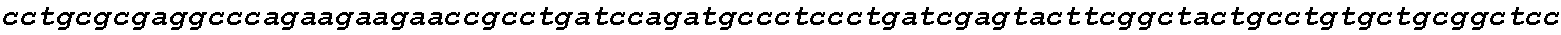

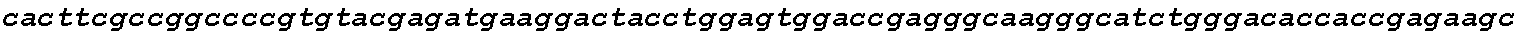

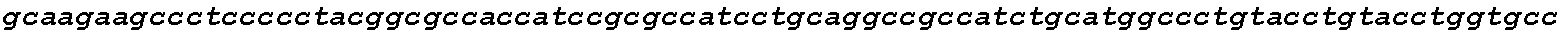

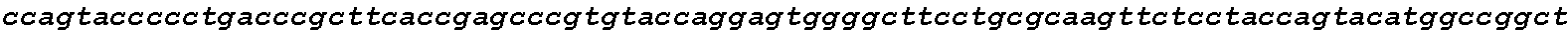

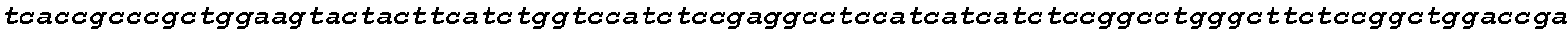

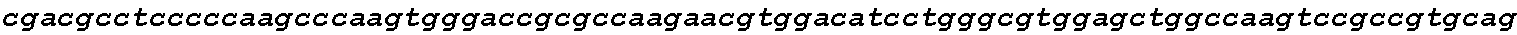

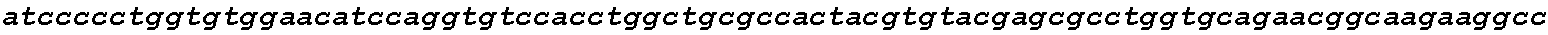

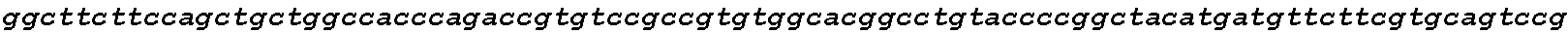

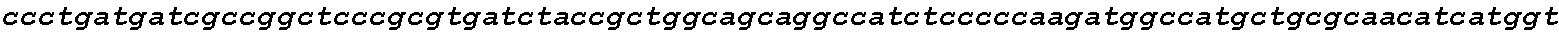

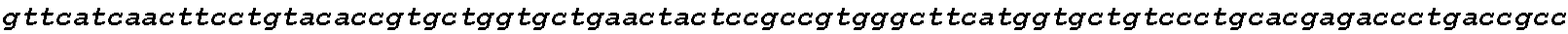

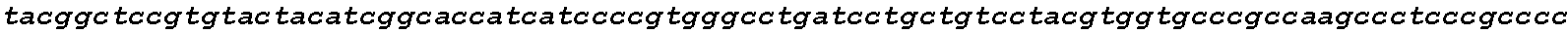

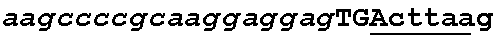

SEQ ID NO: 116

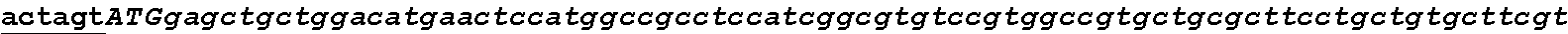

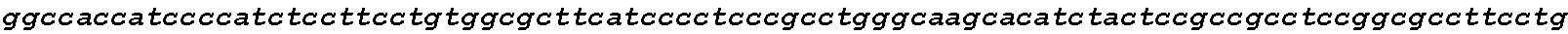

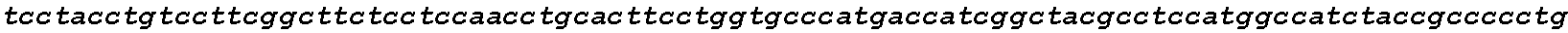

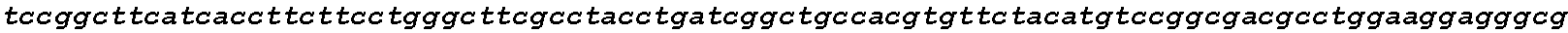

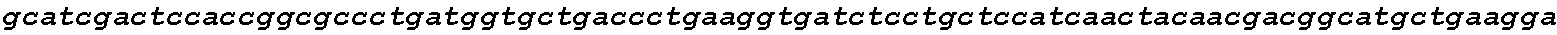

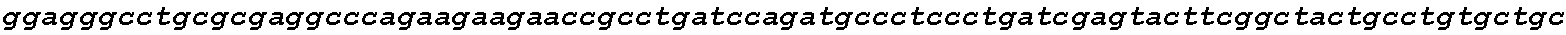

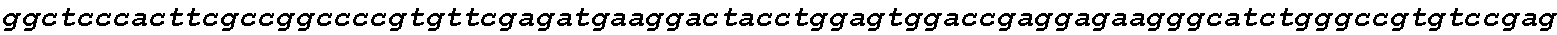

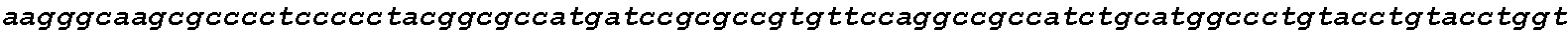

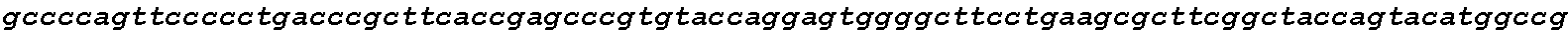

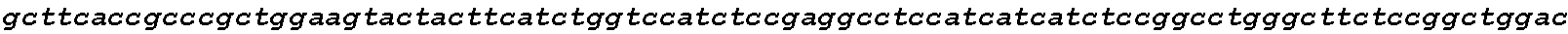

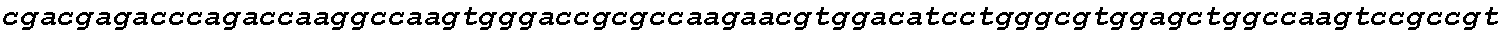

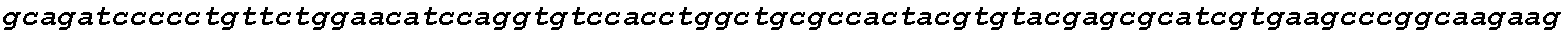

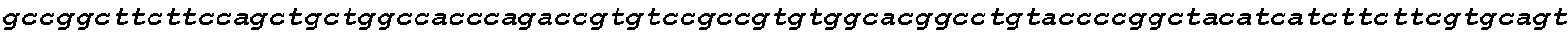

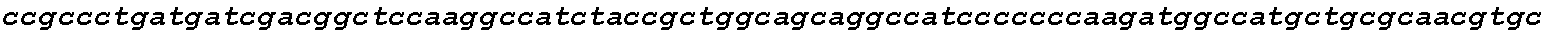

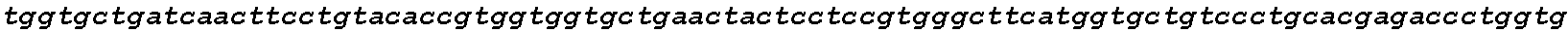

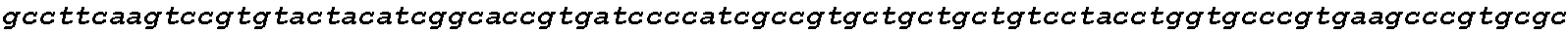

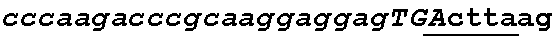

SEQ ID NO: 117
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccattatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg

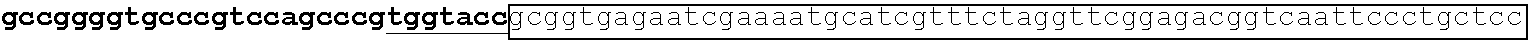

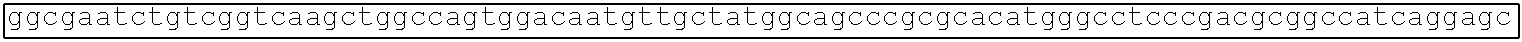

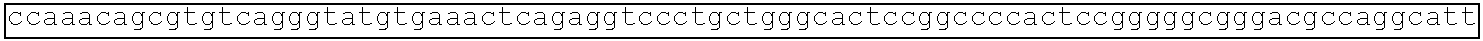

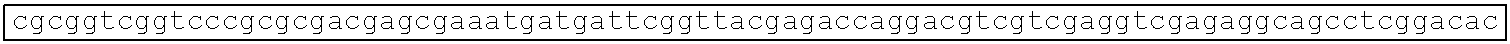

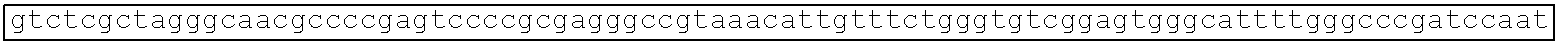

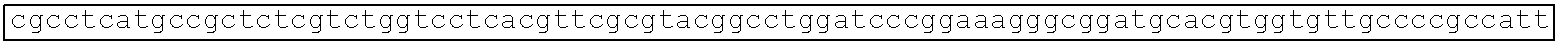

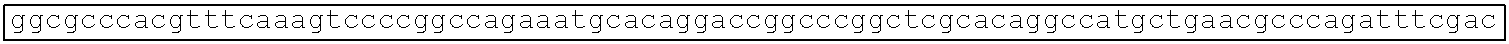

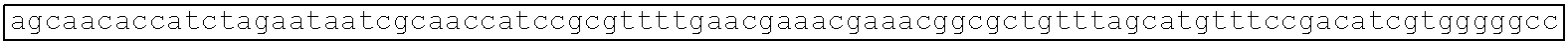

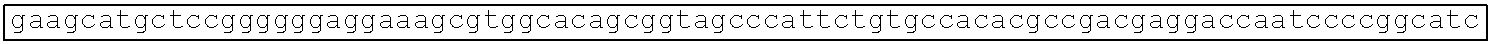

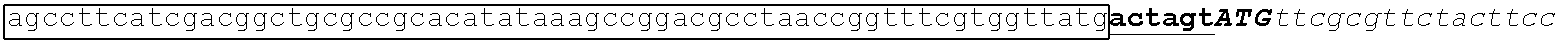

tgacggcctgcatctccctgaagggcgtgttcggcgtctccccctcctacaacggcctgggcctgacgccccagatgggctggga
caactggaacacgttcgcctgcgacgtctccgagcagctgctgctggacacggccgaccgcatctccgacctgggcctgaagga
catgggctacaagtacatcatcctggacgactgctggtcctccggccgcgactccgacggcttcctggtcgccgacgagcagaag
ttccccaacggcatgggccacgtcgccgaccacctgcacaacaactccttcctgacggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgacggccagaagatcggctccctgtcccccaacgcgatcctgaacacg

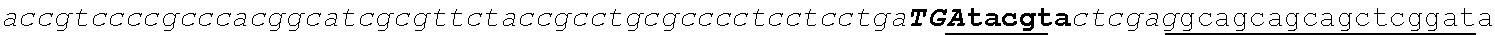

gtatcgacacactctggacgctggtcgtgtgatggactgagccgccacacttgctgccttgacctgtgaatatccctgccgcattatcaa
acagcctcagtgtgatgatcagtgtgtacgcgcattgcgagagctagctgatgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccaggtagggctccgcctgtattctcctggtactgcaacctgtaaaccagcactgcaatgctgatgcacgggaagtagtggg

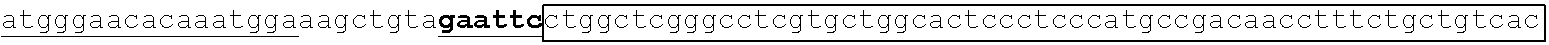

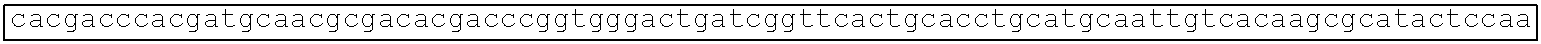

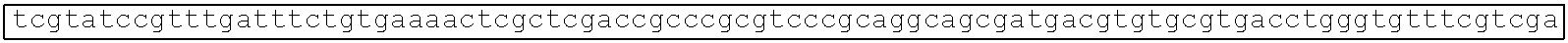

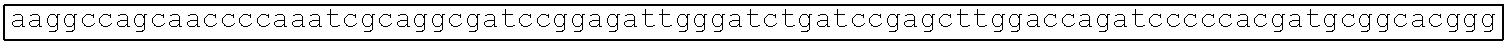

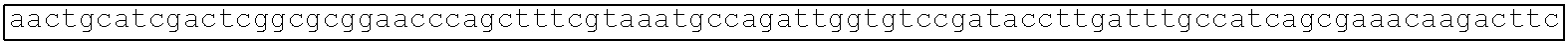

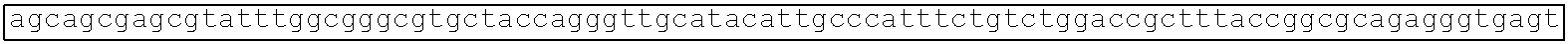

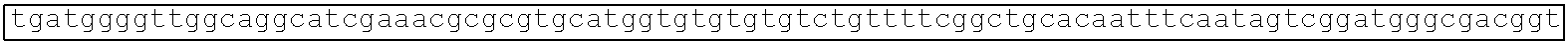

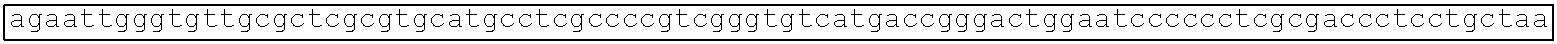

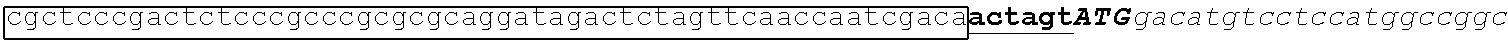

tccatcggcgtgtccgtggccgtgctgcgcttcctgctgtgcttcgtggccaccatccccgtgtccttcgcctgccgcatcgtgccctcc
cgcctgggcaagcacctgtacgccgccgcctccggcgccttcctgtcctacctgtccttcggcttctcctccaacctgcacttcctggt
gcccatgaccatcggctacgcctccatggccatctaccgccccaagtgcggcatcatcaccttcttcctgggcttcgcctacctgatc
ggctgccacgtgactacatgtccggcgacgcctggaaggagggcggcatcgactccaccggcgccctgatggtgctgaccctga
aggtgatctcctgctccatgaactacaacgacggcatgctgaaggaggagggcctgcgcgaggcccagaagaagaaccgcct
gatccagatgccctccctgatcgagtacttcggctactgcctgtgctgcggctcccacttcgccggccccgtgtacgagatgaagga
ctacctggagtggaccgagggcaagggcatctgggacaccaccgagaagcgcaagaagccctccccctacggcgccaccatc
cgcgccatcctgcaggccgccatctgcatggccctgtacctgtacctggtgccccagtaccccctgacccgcttcaccgagcccgt
gtaccaggagtggggcttcctgcgcaagttctcctaccagtacatggccggcttcaccgcccgctggaagtactacttcatctggtc
catctccgaggcctccatcatcatctccggcctgggcttctccggctggaccgacgacgcctcccccaagcccaagtgggaccgc
gccaagaacgtggacatcctgggcgtggagctggccaagtccgccgtgcagatccccctggtgtggaacatccaggtgtccacc
tggctgcgccactacgtgtacgagcgcctggtgcagaacggcaagaaggccggcttatccagctgctggccacccagaccgtgt
ccgccgtgtggcacggcctgtaccccggctacatgatgacttcgtgcagtccgccctgatgatcgccggctcccgcgtgatctacc
gctggcagcaggccatctcccccaagatggccatgctgcgcaacatcatggtgttcatcaacttcctgtacaccgtgctggtgctga
actactccgccgtgggcttcatggtgctgtccctgcacgagaccctgaccgcctacggctccgtgtactacatcggcaccatcatcc

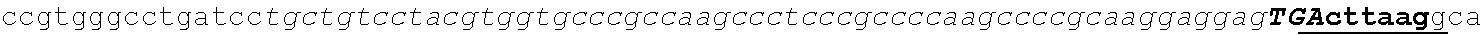

gcagcagctcggatagtatcgacacactctggacgctggtcgtgtgatggactgttgccgccacacttgctgccttgacctgtgaatatc
cctgccgcattatcaaacagcctcagtgtgatgatcagtgtgtacgcgcattgcgagagctagctgcttgtgctatttgcgaataccac
ccccagcatcccatccctcgatcatatcgcttgcatcccaaccgcaacttatctacgctgtcctgctatccctcagcgctgctcctgctc
ctgctcactgcccctcgcacagccaggtagggctccgcctgtattctcctggtactgcaacctgtaaaccagcactgcaatgctgatgc
acgggaagtagtgggatgggaacacaaatggaaagcttaattaagagctccgtcctccactaccacagggtatggtcgtgtgggg
tcgagcgtgttgaagcgcagaaggggatgcgccgtcaagatcaggagctaaaaatggtgccagcgaggatccagcgctctc
actcttgctgccatcgctcccacccttttccccaggggaccctgtggcccacgtgggagacgattccggccaagtggcacatctt
cctgatgctctgccacccccgccacaaagtgaccgtgatgaaggttaggacaagggtcgggacccgattctggatatgacctc
tgaggtgtglltctcgcgcaagcgtcccccaattcgttacaccacatccctcacaccctcgcccctgacactcgcagttgcccgt
gtacgtccccaatgaggaggaaaaggccgaccccaagctgtacgcccaaaacgtccgcaaagccatggtgcgtcgggaacc
gtcaaagtttgcttgcgggtgggcggggcggctctagcgaattggctcattggccctcaccgaggcagcacatcggacaccag
tcgccacccggcttgcatcttcgccccctttcttctcgcagatggaggtcgccgggaccaaggacacgacggcggtgtttgagg
acaagatgcgctacctgaactccctgaagagaaagtacggcaagcctgtgcctaagaaaattgagtgaacccccgtcgtcga
ccagaagagc
SEQ ID NO: 118
Gctcttctgcttcggattccactacatcaagtgggtgaacctggcgggcgcggaggagggccmcgcccgggcggcattgtta
gcaaccactgcagctacctggacatcctgctgcacatgtccgactccttccccgcctttgtggcgcgccagtcgacggccaagc
tgccdttatcggcatcatcaggtgcgtgaaagcgggggctgctgtggccgtggtgggcagggttgcgaaggggggcaggcg
taggcgtgcagtgtgagcggacattgatgccgtcgtttgccggtcaggagagctcgaaatcagagccagcctggtcatgggat
cacagagctcaccaccactcgtccacctcgcctmccttgcagccaaatcatgagdgcctctacgtgaaccgcgaccgctc
ggggcccaaccacgtgggcgtggccgatctggtgaagcagcgcatgcaggacgaggccgaggggaggaccccgcccgagt
accgaccgctgctcctcttccccgaggtgggctttcgaggcaccgtttgtgcttgaaactgtgggcacgcgtgccccgacgcgc
ctctggcgcctgcttcgcatccattcgcctdcaaccccgtctctcctttcctccatcgccagggcaccacctccaacggcgacta
cctgcttcccttcaagaccggcgccttcctggccggggtgcccgtccagcccgtggtacc
SEQ ID NO: 119
Gagctccgtcctccactaccacagggtatggtggtgtggggtcgagcgtgttgaagcgcggaaggggatgagtcgctgtcaagttt
tggagctgaaaatggtgcccgcgaggatccagcgcgccccactcacccttgctgccatcgctccccacccttttccccagggaa
ccctgtggcccacgtgggagacgattccggccaagtggcacatcttcctgatgctctgccacccccgccacaaagtgaccgtg
atgaaggtacgaacaagggtcgggccccgattctggatatcacgtctggggtgtgtttctcgcgcacgcgtcccccgatgcgct
gcacagtctccctcacaccctcacccctaacgctcgcagttgcccgtgtacgtccccaatgaggaggaaaaggccgaccccaa
gctgtacgcccaaaatgttcgcaaagccatggtgcgtcgggaaccgttcaagtttgcttgcgggtgggcggggcggctctagc
gaattggcgcattggccctcaccgaggcagcacatcggacaccaatcgtcacccggcgagcaattccgccccctctgtcttctc
gcagatggaggtcgccgggaccaaggacacgacggcggtgtttgaggacaagatgcgctacctgaactccctgaagagaaa
gtacggcaagcctgtgcctaagaaaattgagtgaacccccgtcgtcgaccagaagagc
SEQ ID NO: 120

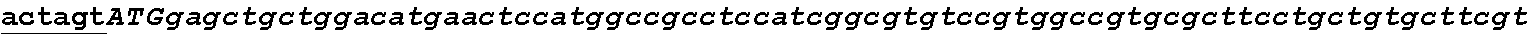

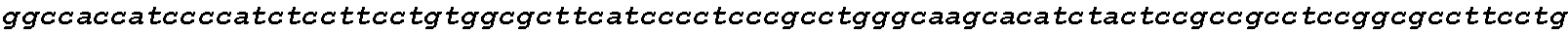

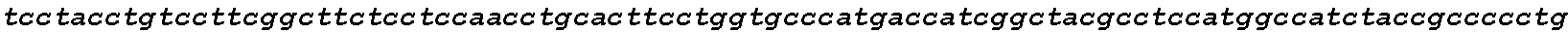

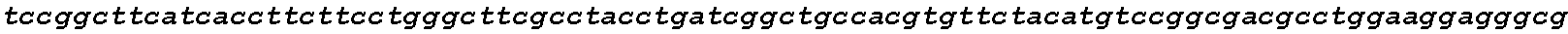

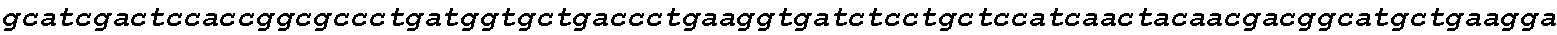

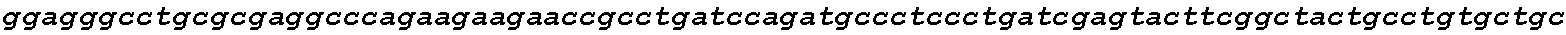

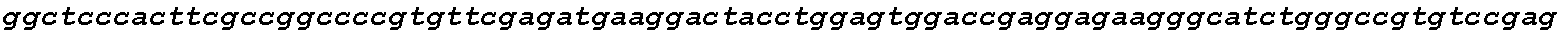

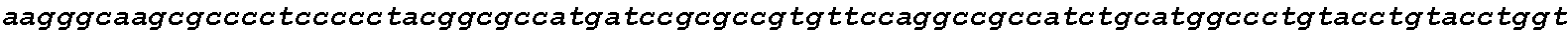

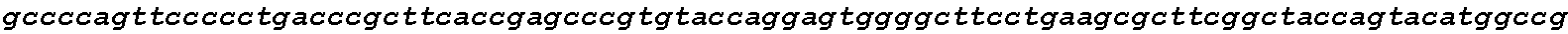

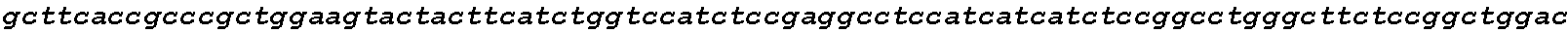

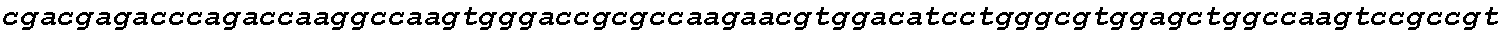

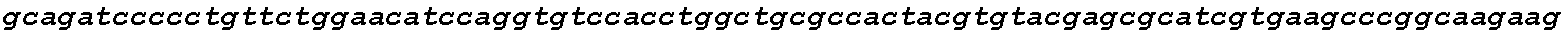

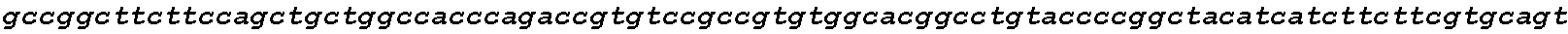

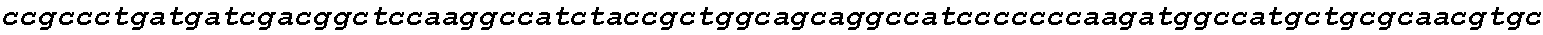

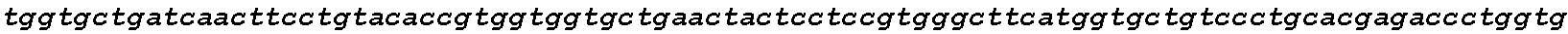

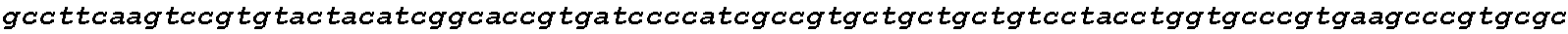

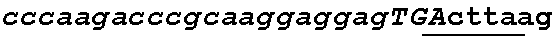

SEQ ID NO: 121

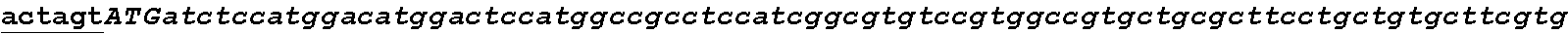

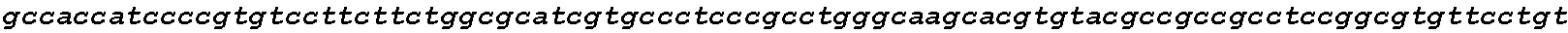

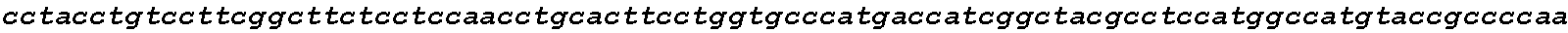

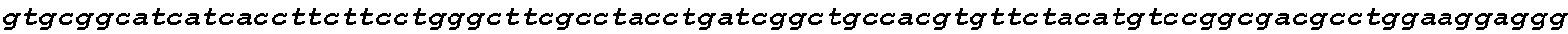

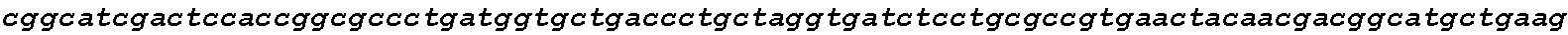

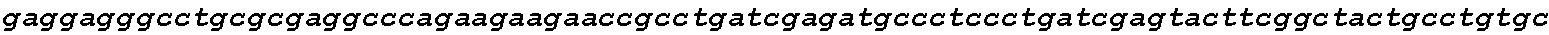

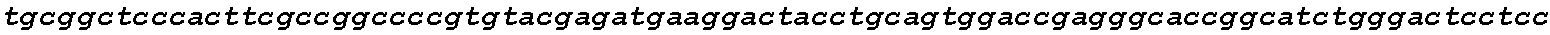

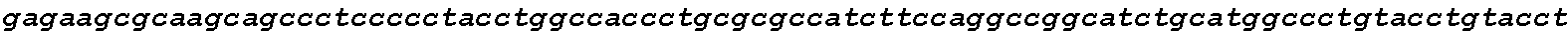

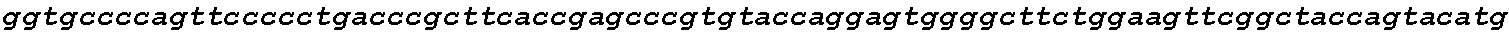

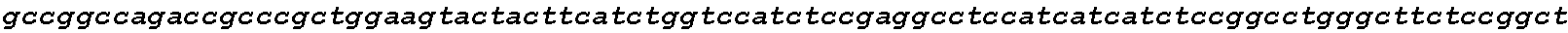

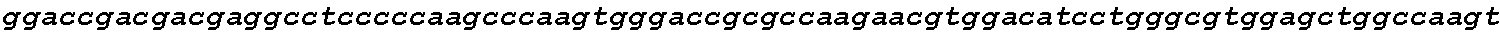

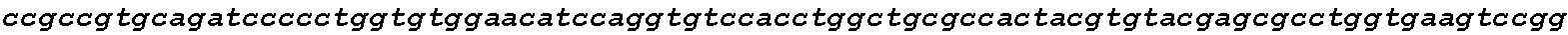

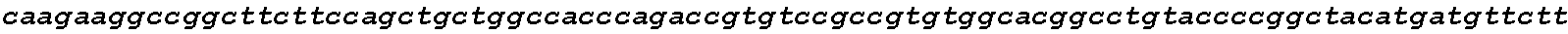

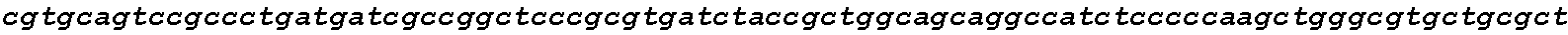

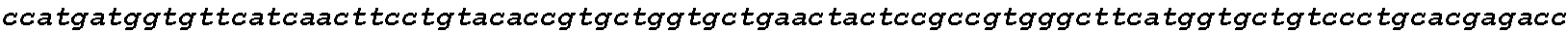

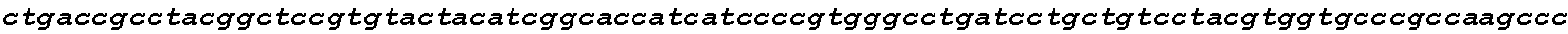

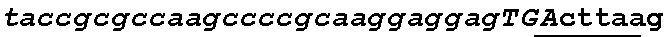

SEQ ID NO: 122

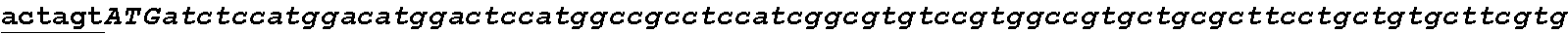

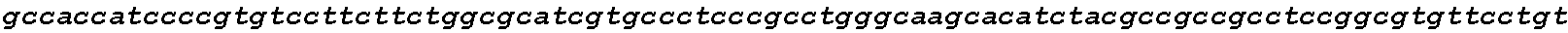

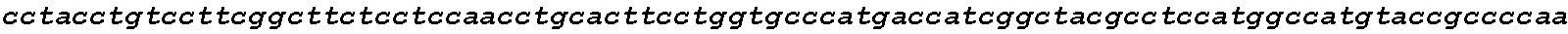

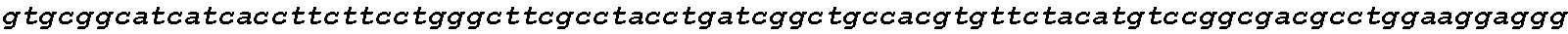

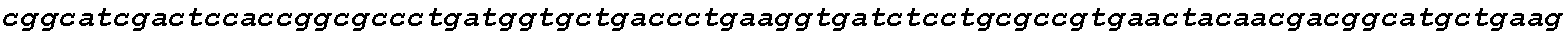

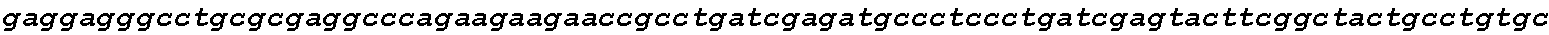

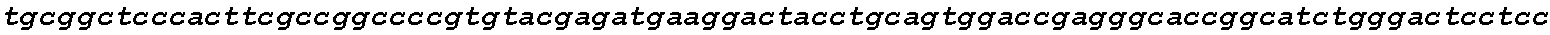

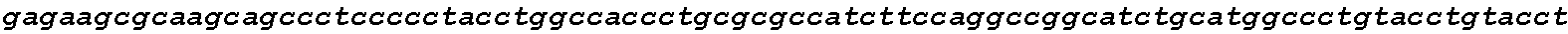

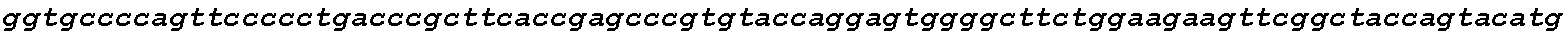

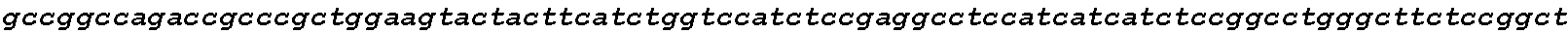

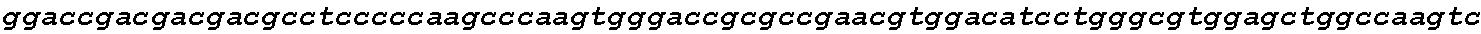

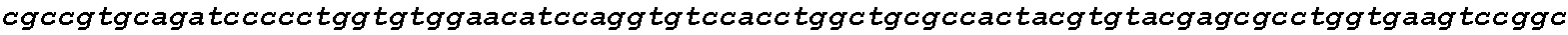

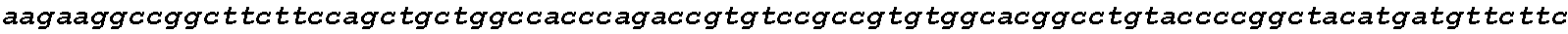

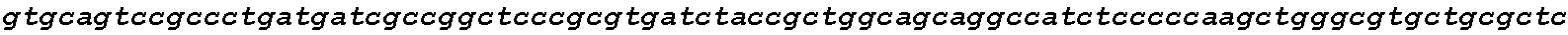

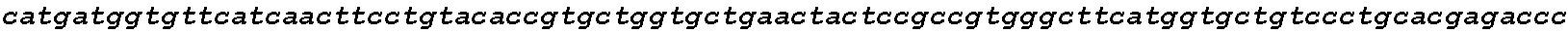

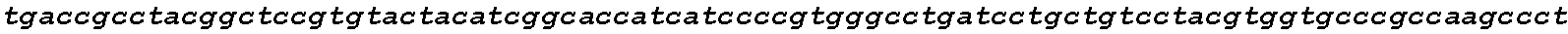

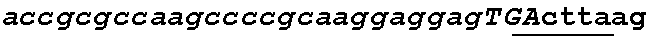

SEQ ID NO: 123

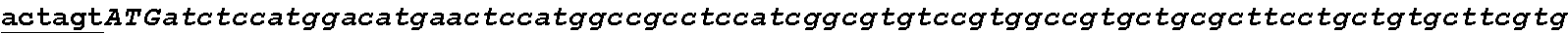

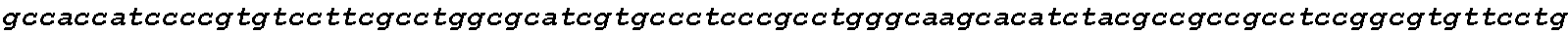

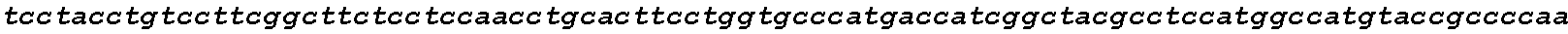

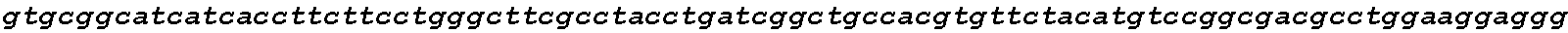

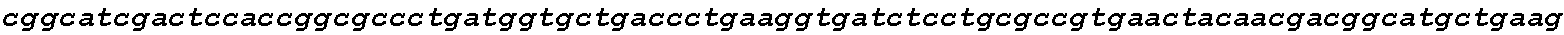

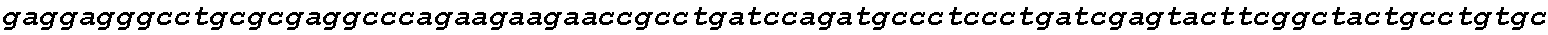

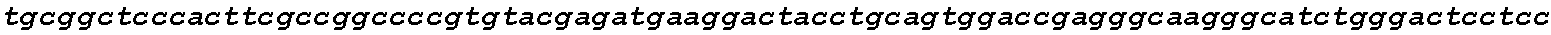

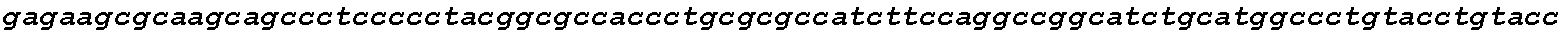

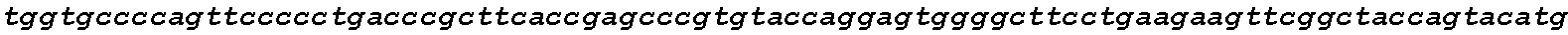

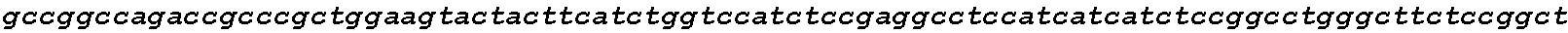

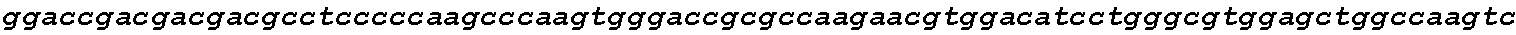

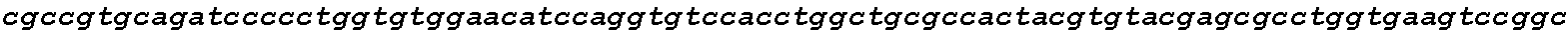

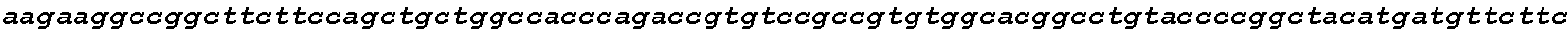

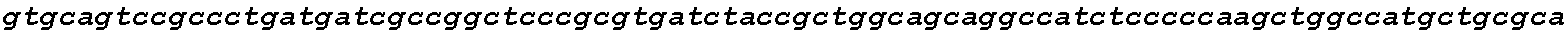

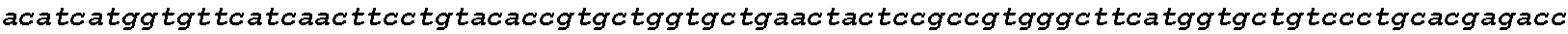

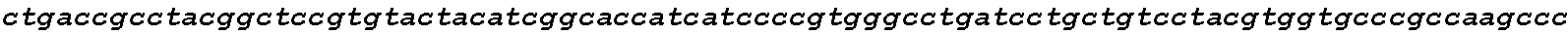

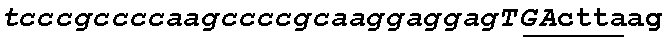

SEQ ID NO: 124

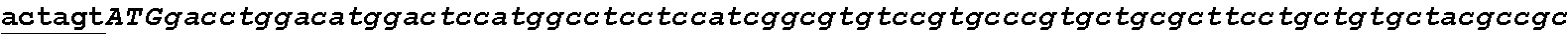

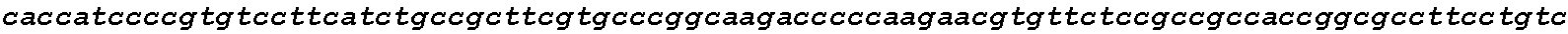

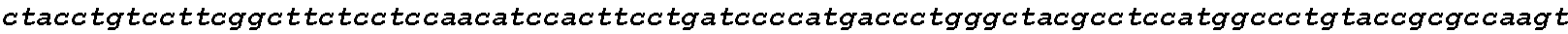

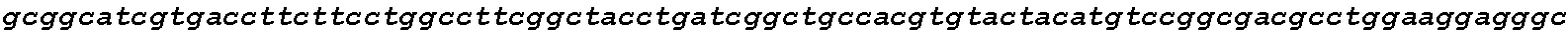

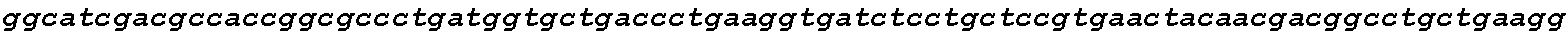

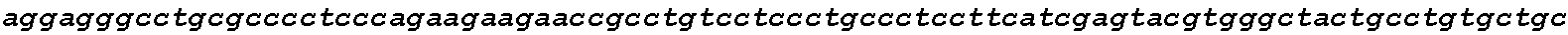

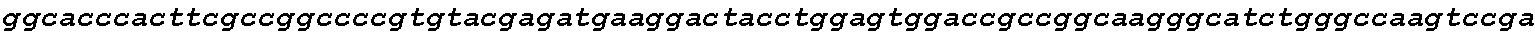

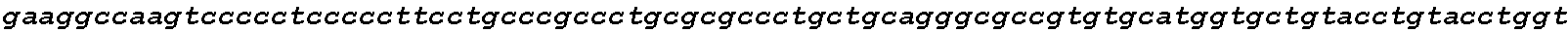

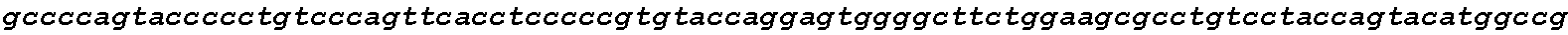

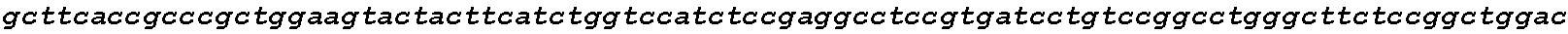

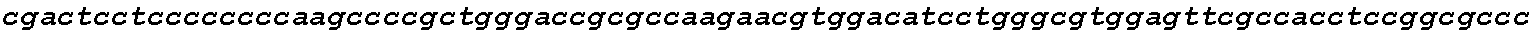

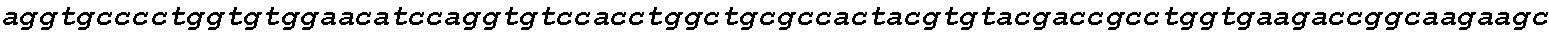

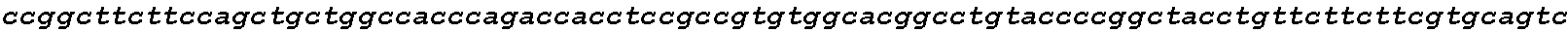

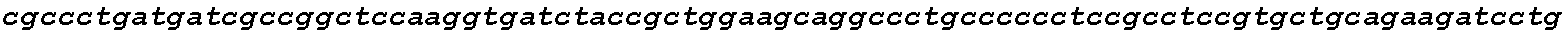

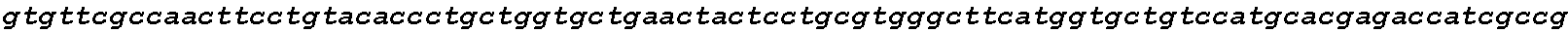

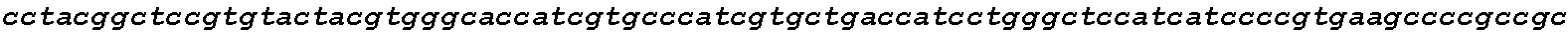

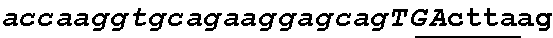

SEQ ID NO: 125

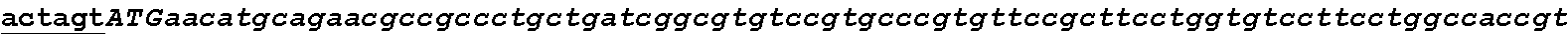

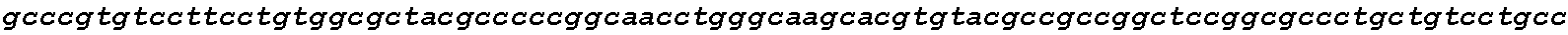

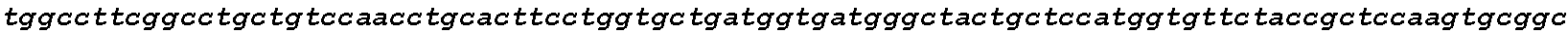

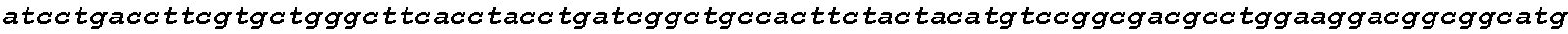

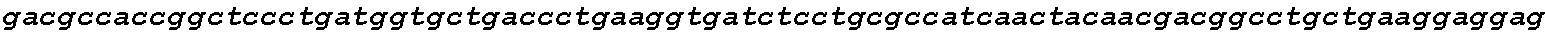

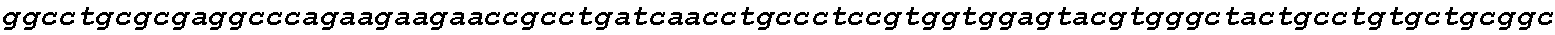

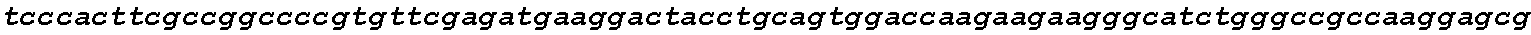

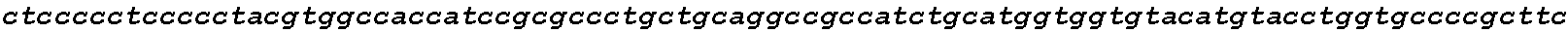

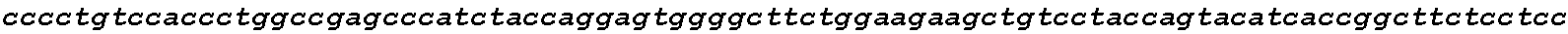

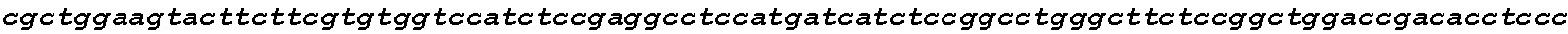

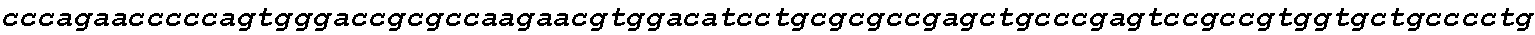

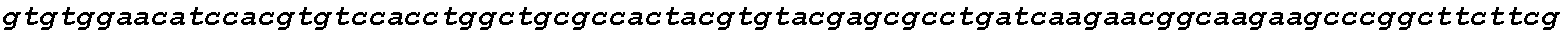

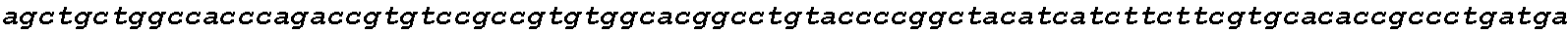

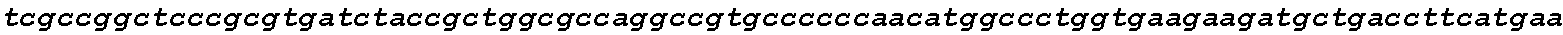

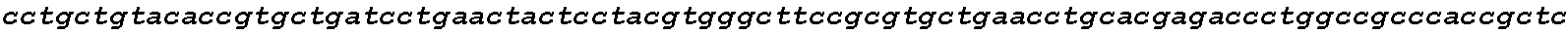

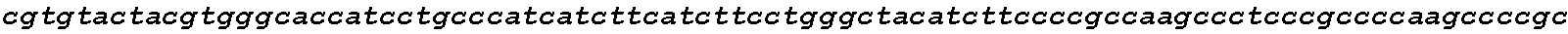

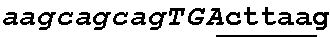

SEQ ID NO: 126
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccdttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccgmcctgtdcgcatccattcgcctct
caaccccatctcaccttttctccatcgccagggcaccacctccaacggcgactacctgcttcccttcaagaccggcgccttcctg

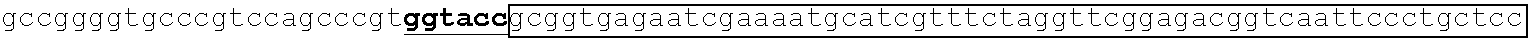

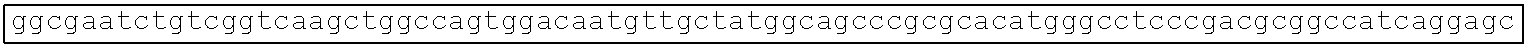

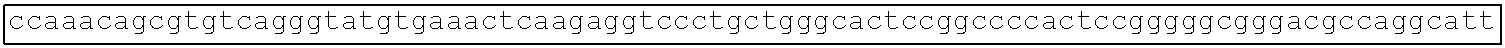

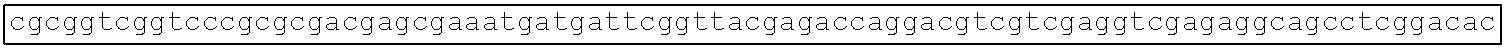

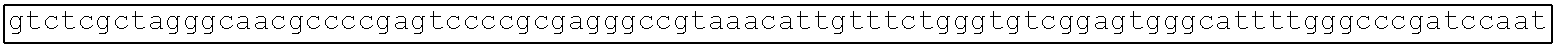

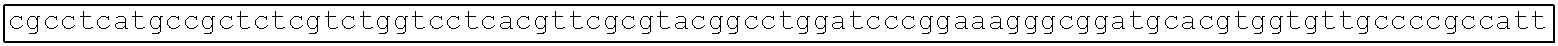

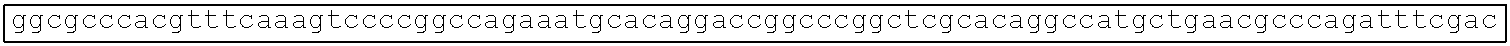

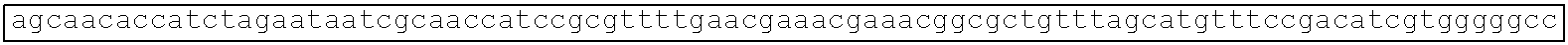

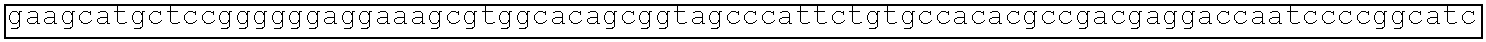

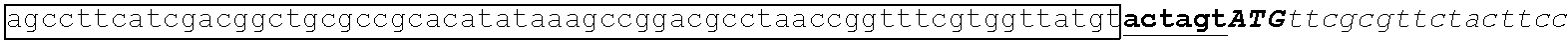

tgacggcctgcatctccctgaagggcgtgttcggcgtctccccctcctacaacggcctgggcctgacgccccagatgggctggga
caactggaacacgttcgcctgcgacgtctccgagcagctgctgctggacacggccgaccgcatctccgacctgggcctgaagga
catgggctacaagtacatcatcctggacgactgctggtcctccggccgcgactccgacggcttcctggtcgccgacgagcagaag
ttccccaacggcatgggccacgtcgccgaccacctgcacaacaactccttcctgttcggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgttcggccagaagatcggctccctgtcccccaacgcgatcctgaacacg

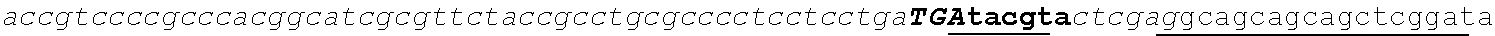

gtatcgacacactctggacgctggtcgtgtgatggactgttgccgccacacttgctgccttgacctgtgaatatccctgccgcattatcaa
acagcctcagtgtgatgatcagtgtgtacgcgcattgcgagagctagctgcttgtgctatttgcgaataccacccccagcatccccttc
cctcgatcatatcgcttgcatcccaaccgcaacttatctacgctgtcctgctatccctcagcgctgctcctgctcctgctcactgcccctcg
cacagccaggtagggctccgcctgtattctcctggtactgcaacctgtaaaccagcactgcaatgctgatgcacgggaagtagtggg

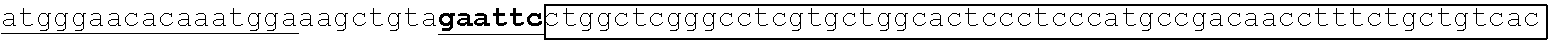

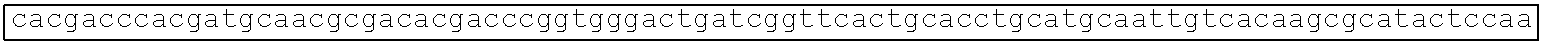

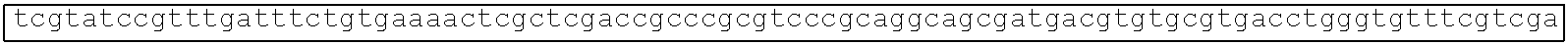

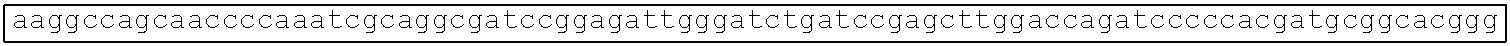

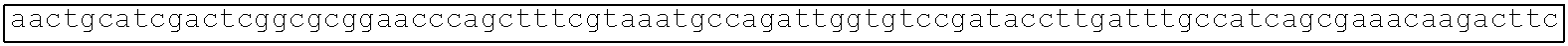

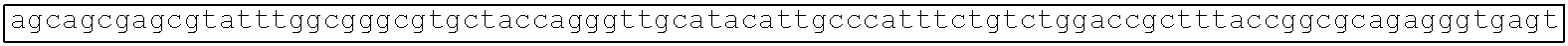

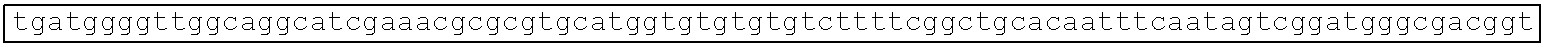

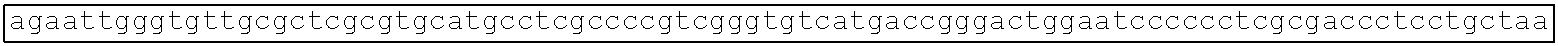

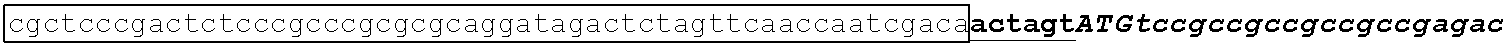

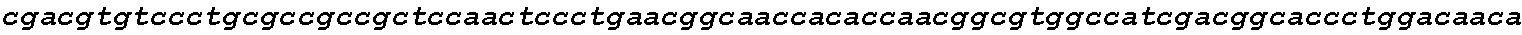

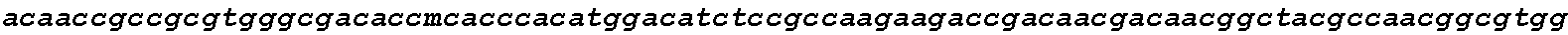

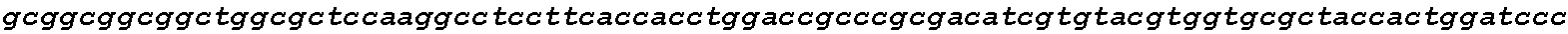

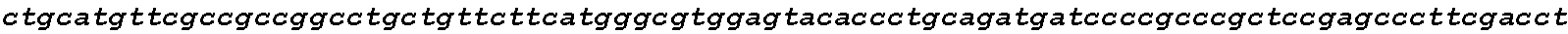

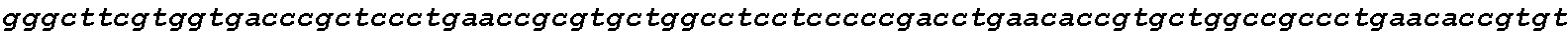

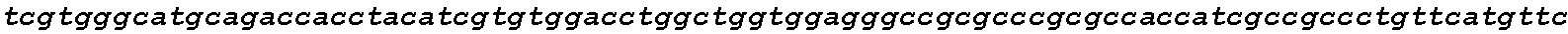

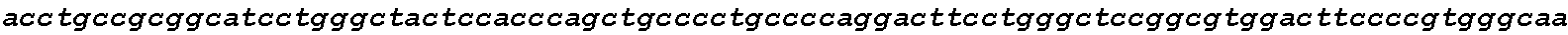

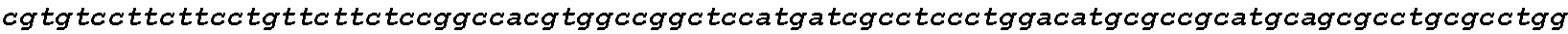

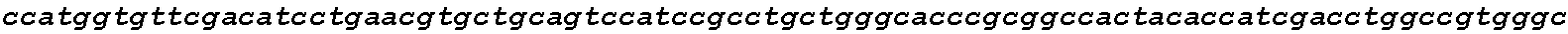

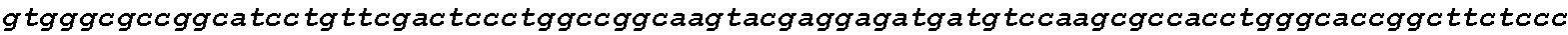

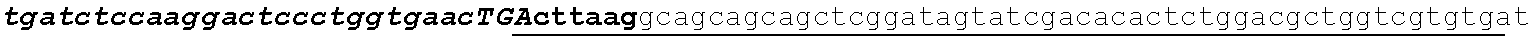

ggacgttgccgccacacttgctgccttgacctgtgaatatccctgccgcattatcaaacagcctcagtgtgatgatcagtgtgtacgcg
cttttgcgagagctagctgcttgtgctatttgcgaataccacccccagcatcccatccctcgatcatatcgcttgcatcccaaccgcaac
ttatctacgctgtcctgctatccctcagcgctgctcctgctcctgctcactgcccctcgcacagccaggtagggctccgcctgtattctcc
tggtactgcaacctgtaaaccagcactgcaatgctgatgcacgggaagtagtgggatgggaacacaaatggaaagcttaattaagag
ctccgtcctccactaccacagggtatggtcgtgtggggtcgagcgtgttgaagcgcagaaggggatgcgccgtcaagatcag
gagctaaaaatggtgccagcgaggatccagcgctctcactcttgctgccatcgctcccacccttttccccaggggaccctgtgg
cccacgtgggagacgattccggccaagtggcacatcttcctgatgctctgccacccccgccacaaagtgaccgtgatgaaggt
taggacaagggtcgggacccgattctggatatgacctctgaggtgtgtttctcgcgcaagcgtcccccaattcgttacaccaca
tccctcacaccctcgcccctgacactcgcagttgcccgtgtacgtccccaatgaggaggaaaaggccgaccccaagctgtacg
cccaaaacgtccgcaaagccatggtgcgtcgggaaccgtcaaagtttgcttgcgggtgggcggggcggctctagcgaattgg
ctcattggccctcaccgaggcagcacatcggacaccagtcgccacccggcttgcatcttcgccccctttcttctcgcagatggag
gtcgccgggaccaaggacacgacggcggtgtttgaggacaagatgcgctacctgaactccctgaagagaaagtacggcaag
cctgtgcctaagaaaattgagtgaacccccgtcgtcgaccagaagagc
SEQ ID NO: 127
gctcttctgcttcggattccactacatcaagtgggtgaacctggcgggcgcggaggagggcccccgcccgggcggcattgtta
gcaaccactgcagctacctggacatcctgctgcacatgtccgactccttccccgcctttgtggcgcgccagtcgacggccaagc
tgccctttatcggcatcatcaggtgcgtgaaagcgggggctgctgtggccgtggtgggcagggttgcgaaggggggcaggcg
taggcgtgcagtgtgagcggacattgatgccgtcgtttgccggtcaggagagctcgaaatcagagccagcctggtcatgggat
cacagagctcaccaccactcgtccacctcgcctgcgccttgcagccaaatcatgagctgcctctacgtgaaccgcgaccgctc
ggggcccaaccacgtgggcgtggccgatctggtgaagcagcgcatgcaggacgaggccgaggggaggaccccgcccgagt
accgaccgctgctcctcttccccgaggtgggctttcgaggcaccgtttgtgcttgaaactgtgggcacgcgtgccccgacgcgc
ctctggcgcctgcttcgcatccattcgcctctcaaccccgtctctcctttcctccatcgccagggcaccacctccaacggcgacta
cctgcttcccttcaagaccggcgccttcctggccggggtgcccgtccagcccgtggtacc
SEQ ID NO: 128
gagctccgtcctccactaccacagggtatggtggtgtggggtcgagcgtgttgaagcgcggaaggggatgcgctgtcaagttt
tggagctgaaaatggtgcccgcgaggatccagcgcgccccactcacccttgctgccatcgctccccacccttttccccagggaa
ccctgtggcccacgtgggagacgattccggccaagtggcacatcttcctgatgctctgccacccccgccacaaagtgaccgtg
atgaaggtacgaacaagggtcgggccccgattctggatatcacgtctggggtgtgtttctcgcgcacgcgtcccccgatgcgct
gcacagtctccctcacaccctcacccctaacgctcgcagttgcccgtgtacgtccccaatgaggaggaaaaggccgaccccaa
gctgtacgcccaaaatgttcgcaaagccatggtgcgtcgggaaccgttcaagtttgcttgcgggtgggcggggcggctctagc
gaattggcgcattggccctcaccgaggcagcacatcggacaccaatcgtcaccggcgagcaattccgccccctctgtcttctc
gcagatggaggtcgccgggaccaaggacacgacggcggtgtttgaggacaagatgcgctacctgaactccctgaagagaaa
gtacggcaagcctgtgcctaagaaaattgagtgaacccccgtcgtcgaccagaagagc
SEQ ID NO: 129
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccctttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccac
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg
gccggggtgcccgtccagcccgtggtaccgcggtgagaatcgaaaatgcatcgtttctaggttcggagacggtcaattccctgctcc
ggcgaatctgtcggtcaagctggccagtggacaatgttgctatggcagcccgcgcacatgggcctcccgacgcggccatcaggagc
ccaaacagcgtgtcagggtatgtgaaactcaagaggtccctgctgggcactccggccccactccgggggcgggacgccaggcattc
gcggtcggtcccgcgcgacgagcgaaatgatgattcggttacgagaccaggacgtcgtcgaggtcgagaggcagcctcggacacg
tctcgctagggcaacgccccgagtccccgcgagggccgtaaacattgtttctgggtgtcggagtgggcattttgggcccgatccaatc
gcctcatgccgctctcgtctggtcctcacgttcgcgtacggcctggatcccggaaagggcggatgcacgtggtgttgccccgccattg
gcgcccacgtttcaaagtccccggccagaaatgcacaggaccggcccggctcgcacaggccatgctgaacgcccagatttcgaca
gcaacaccatctagaataatcgcaaccatccgcgttttgaacgaaacgaaacggcgctgtttagcatgtttccgacatcgtgggggccg
aagcatgctccggggggaggaaagcgtggcacagcggtagcccattctgtgccacacgccgacgaggaccaatccccggcatca

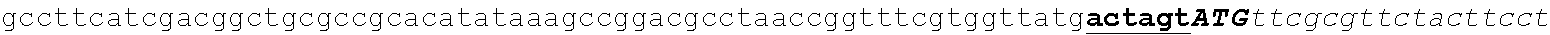

gacggcctgcatctccctgaagggcgtgttcggcgtctccccctcctacaacggcctgggcctgacgccccagatgggctgggac
aactggaacacgttcgcctgcgacgtctccgagcagctgctgctggacacggccgaccgcatctccgacctgggcctgaaggac
atgggctacaagtacatcatcctggacgactgctggtcctccggccgcgactccgacggcttcctggtcgccgacgagcagaagt
tccccaacggcatgggccacgtcgccgaccacctgcacaacaactccttcctgttcggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgttcggccagaagatcggctccctgtcccccaacgcgatcctgaacacg

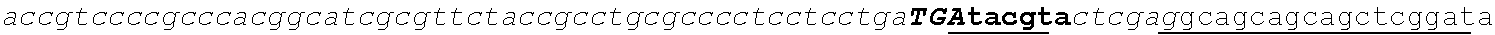

gtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtggg
atgggaacacaaatggaaagctgtagaattcctggctcgggcctcgtgctggcactccctcccatgccgacaacctttctgctgtcacc
acgacccacgatgcaacgcgacacgacccggtgggactgatcggttcactgcacctgcatgcaattgtcacaagcgcatactccaat
cgtatccgtttgatttctgtgaaaactcgctcgaccgcccgcgtcccgcaggcagcgatgacgtgtgcgtgacctgggtgtttcgtcga
aaggccagcaaccccaaatcgcaggcgatccggagattgggatctgatccgagcttggaccagatcccccacgatgcggcacggg
aactgcatcgactcggcgcggaacccagctttcgtaaatgccagattggtgtccgataccttgatttgccatcagcgaaacaagacttca
gcagcgagcgtatttggcgggcgtgctaccagggttgcatacattgcccatttctgtctggaccgctttaccggcgcagagggtgagtt
gatggggttggcaggcatcgaaacgcgcgtgcatggtgtgtgtgtctgttttcggctgcacaatttcaatagtcggatgggcgacggta
gaattgggtgttgcgctcgcgtgcatgcctcgccccgtcgggtgtcatgaccgggactggaatcccccctcgcgaccctcctgctaac

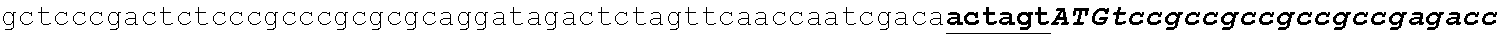

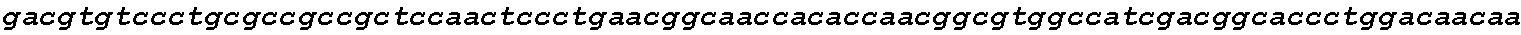

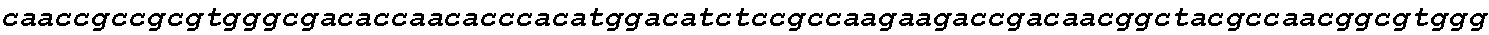

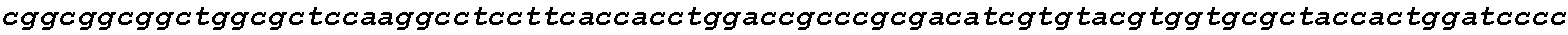

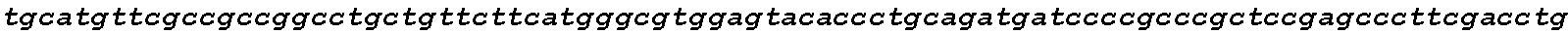

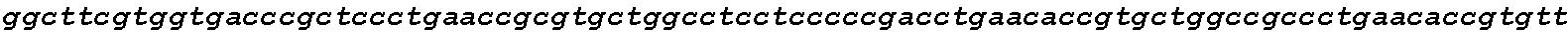

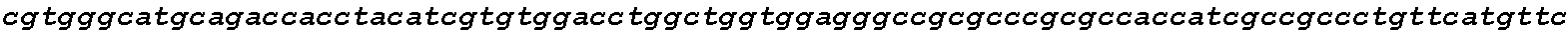

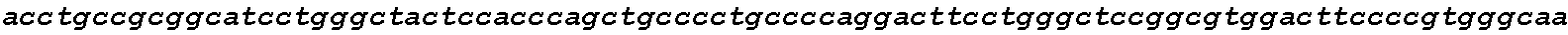

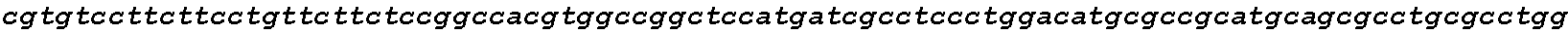

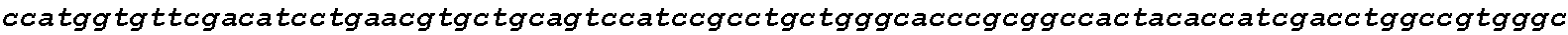

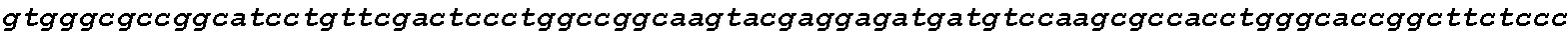

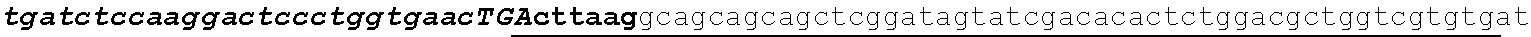

ggactgttgccgccacacttgctgccttgacctgtgaatatccctgccgcttttatcaaacagcctcagtgtgtttgatcttgtgtgtacgcg
cttttgcgagttgctagctgcttgtgctatttgcgaataccacccccagcatccccttccctgtttcatatcgcttgcatcccaaccgcaac
ttatctacgctgtcctgctatccctcagcgctgctcctgctcctgctcactgcccctcgcacagccttggtttgggctccgcctgtattctcc
tggtactgcaacctgtaaaccagcactgcaatgctgatgcacgggaagtagtgggatgggaacacaaatggaaagcttaattaagag
ctccgtcctccactaccacagggtatggtcgtgtggggtcgagcgtgttgaagcgcagaaggggatgcgccgtcaagatcag
gagctaaaaatggtgccagcgaggatccagcgctctcactcttgctgccatcgctcccacccttttccccaggggaccctgtgg
cccacgtgggagacgattccggccaagtggcacatcttcctgatgctctgccacccccgccacaaagtgaccgtgatgaaggt
taggacaagggtcgggacccgattctggatatgacctctgaggtgtgtttctcgcgcaagcgtcccccaattcgttacaccaca
tccctcacaccctcgcccctgacactcgcagttgcccgtgtacgtccccaatgaggaggaaaaggccgaccccaagctgtacg
cccaaaacgtccgcaaagccatggtgcgtcgggaaccgtcaaagtttgcttgcgggtgggcggggcggctctagcgaattgg
ctcattggccctcaccgaggcagcacatcggacaccagtcgccacccggcttgcatcttcgccccctttcttctcgcagatggag
gtcgccgggaccaaggacacgacggcggtgtttgaggacaagatgcgctacctgaactccctgaagagaaagtacggcaag
cctgtgcctaagaaaattgagtgaacccccgtcgtcgaccagaagagc
SEQ ID NO: 130
gctcttctgcttcggattccactacatcaagtgggtgaacctggcgggcgcggaggagggcccccgcccgggcggcattgtta
gcaaccactgcagctacctggacatcctgctgcacatgtccgactccttccccgcctttgtggcgcgccagtcgacggccaagc
tgccctttatcggcatcatcaggtgcgtgaaagcgggggctgctgtggccgtggtgggcagggttgcgaaggggggcaggcg
taggcgtgcagtgtgagcggacattgatgccgtcgtttgccggtcaggagagctcgaaatcagagccagcctggtcatgggat
cacagagctcaccaccactcgtccacctcgcctgcgccttgcagccaaatcatgagctgcctctacgtgaaccgcgaccgctc
ggggcccaaccacgtgggcgtggccgatctggtgaagcagcgcatgcaggacgaggccgaggggaggaccccgcccgagt
accgaccgctgctcctcttccccgaggtgggctttcgaggcaccgtttgtgcttgaaactgtgggcacgcgtgccccgacgcgc
ctctggcgcctgcttcgcatccattcgcctctcaaccccgtctctcctttcctccatcgccagggcaccacctccaacggcgacta
cctgcttcccttcaagaccggcgccttcctggccggggtgcccgtccagcccgtggtacc
SEQ ID NO: 131
gagctcgtcctccactaccacagggtatggtggtgtggggtcgagcgtgttgaagcgcggaaggggatgcgctgtcaagttt
tggagctgaaaatggtgcccgcgaggatccagcgcgccccactcacccttgctgccatcgctccccacccttttccccagggaa
ccctgtggcccacgtgggagacgattccggccaagtggcacatcttcctgatgctctgccacccccgccacaaagtgaccgtg
atgaaggtacgaacaagggtcgggccccgattctggatatcacgtctggggtgtgtttctcgcgcacgcgtcccccgatgcgct
gcacagtctccctcacaccctcacccctaacgctcgcagttgcccgtgtacgtccccaatgaggaggaaaaggccgaccccaa
gctgtacgcccaaaatgttcgcaaagccatggtgcgtcgggaaccgttcaagtttgcttgcgggtgggcggggcggctctagc
gaattggcgcattggccctcaccgaggcagcacatcggacaccaatcgtcacccggcgagcaattccgccccctctgtcttctc
gcagatggaggtcgccgggaccaaggacacgacggcggtgtttgaggacaagatgcgctacctgaactccctgaagagaaa
gtacggcaagcctgtgcctaagaaaattgagtgaacccccgtcgtcgaccagaagagc
SEQ ID NO: 132
gctcttctgcttcggattccactacatcaagtgggtgaacctggcgggcgcggaggagggcccccgcccgggcggcattgtta
gcaaccactgcagctacctggacatcctgctgcacatgtccgattccttccccgcctttgtggcgcgccagtcgacggccaagc
tgccctttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg
gccggggtgcccgtccagcccgtggtaccgcgcggtgagaatcgaaaatgcatcgtttctaggttcggagacggtcaattccctgctcc
ggcgaatctgtcggtcaagctggccagtggacaatgttgctatggcagcccgcgcacatgggcctcccgacgcggccatcaggagc
ccaaacagcgtgtcagggtatgtgaaactcaagaggtccctgctgggcactccggccccactccgggggcgggacgccaggcattc
gcggtcggtcccgcgcgacgagcgaaatgatgattcggttacgagaccaggacgtcgtcgaggtcgagaggcagcctcggacacg
tctcgctagggcaacgccccgagtccccgcgagggccgtaaacattgtttctgggtgtcggagtgggcattttgggcccgatccaatc
gcctcatgccgctctcgtctggtcctcacgttcgcgtacggcctggatcccggaaagggcggatgcacgtggtgttgccccgccattg
gcgcccacgtttcaaagtccccggccagaaatgcacaggaccggcccggctcgcacaggccatgctgaacgcccagatttcgaca
gcaacaccatctagaataatcgcaaccatccgcgttttgaacgaaacgaaacggcgctgtttagcatgtttccgacatcgtgggggccg
aagcatgctccggggggaggaaagcgtggcacagcggtagcccattctgtgccacacgccgacgaggaccaatccccggcatcac

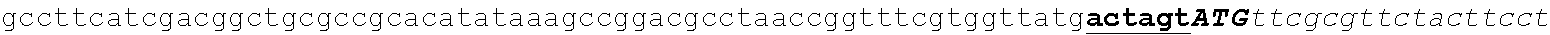

gacggcctgcatctccctgaagggcgtgttcggcgtctccccctcctacaacggcctgggcctgacgccccagatgggctgggac
aactggaacacgttcgcctgcgacgtctccgagcagctgctgctggacacggccgaccgcatctccgacctgggcctgaaggac
atgggctacaagtacatcatcctggacgactgctggtcctccggccgcgactccgacggcttcctggtcgccgacgagcagaagt
tccccaacggcatgggccacgtcgccgaccacctgcacaacaactccttcctgttcggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccctttcctgggctccaagaagctgacctccacctgggacatctacgacctgtgggcgaacc
gcgtcgacaactccacggcgtccgccatcctgggccgcaacaagaccgccaccggcatcctgtacaacgccaccgagcagtcc
tacaaggacggcctgtccaagaacgacacccgcctgucggccagaagatcggctccctgtcccccaacgcgatcctgaacacg

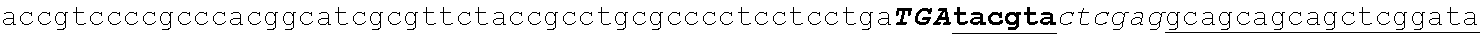

gtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtggg
atgggaacacaaatggaaagctgtagaattcctggctcgggcctcgtgctggcactccctcccatgccgacaacctttctgctgtcacc
acgacccacgatgcaacgcgacacgacccggtgggactgatcggttcactgcacctgcatgcaattgtcacaagcgcatactccaat
cgtatccgtttgatttctgtgaaaactcgctcgaccgcccgcgtcccgcaggcagcgatgacgtgtgcgtgacctgggtgtttcgtcga
aaggccagcaaccccaaatcgcaggcgatccggagattgggatctgatccgagcttggaccagatcccccacgatgcggcacggg
aactgcatcgactcggcgcggaacccagctttcgtaaatgccagattggtgtccgataccttgatttgccatcagcgaaacaagacttca
gcagcgagcgtatttggcgggcgtgctaccagggttgcatacattgcccatttctgtctggaccgctttaccggcgcagagggtgagtt
gatggggttggcaggcatcgaaacgcgcgtgcatggtgtgtgtgtctgttttcggctgcacaatttcaatagtcggatgggcgacggta
gaattgggtgttgcgctcgcgtgcatgcctcgccccgtcgggtgtcatgaccgggactggaatcccccctcgcgaccctcctgctaac

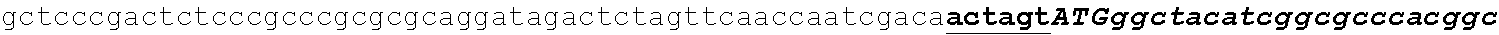

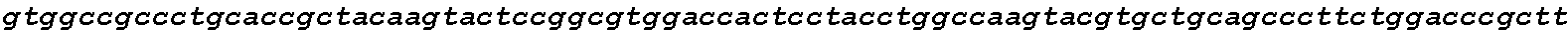

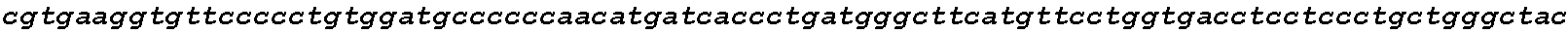

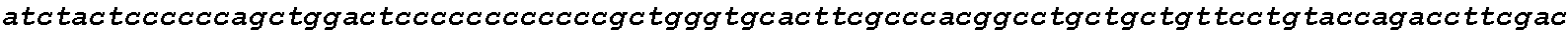

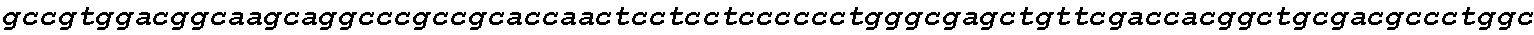

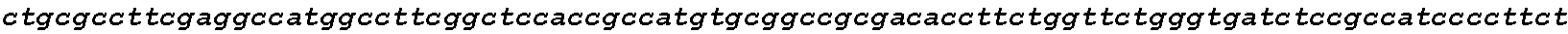

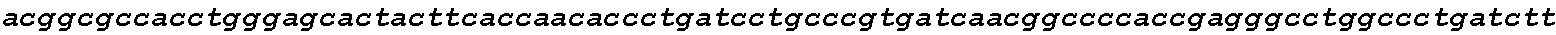

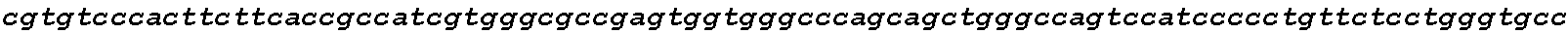

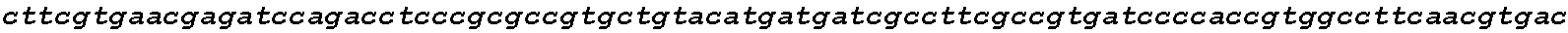

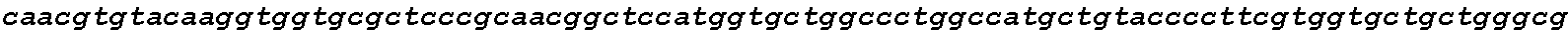

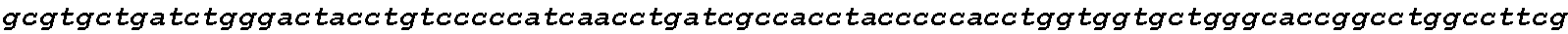

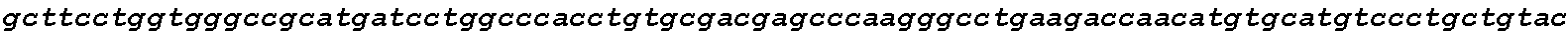

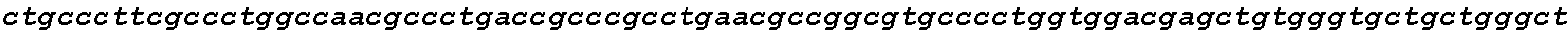

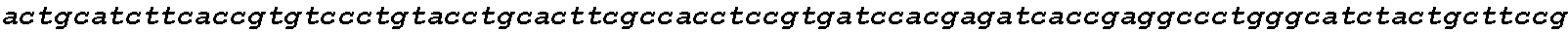

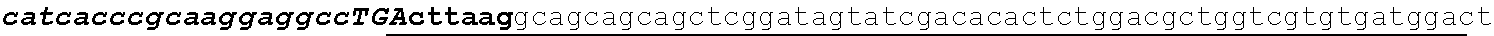

gttgccgccacacttgctgccttgacctgtgaatatccctgccgcttttatcaaacagcctcagtgtgtttgatcttgtgtgtacgcgcttttg
cgagttgctagctgcttgtgctatttgcgaataccacccccagcatccccttccctcgtttcatatcgcttgcatcccaaccgcaacttatct
acgctgtcctgctatccctcagcgctgctcctgctcctgctcactgcccctcgcacagccttggtttgggctccgcctgtattctcctggta
ctgcaacctgtaaaccagcactgcaatgctgatgcacgggaagtagtgggatgggaacacaaatggaaagcttaattaagagctccg
tcctccactaccacagggtatggtcgtgtggggtcgagcgtgttgaagcgcagaaggggatgcgccgtcaagatcaggagct
aaaaatggtgccagcgaggatccagcgctctcactcttgctgccatcgctcccacccttttccccaggggaccctgtggcccac
gtgggagacgattccggccaagtggcacatcttcctgatgctctgccacccccgccacaaagtgaccgtgatgaaggttagga
caagggtcgggacccgattctggatatgacctctgaggtgtgtttctcgcgcaagcgtcccccaattcgttacaccacatccctc
acaccctcgcccctgacactcgcagttgcccgtgtacgtccccaatgaggaggaaaaggccgaccccaagctgtacgcccaa
aacgtccgcaaagccatggtgcgtcgggaaccgtcaaagtttgcttgcgggtgggcggggcggctctagcgaattggctcatt
ggccctcaccgaggcagcacatcggacaccagtcgccacccggcttgcatcttcgccccctttcttctcgcagatggaggtcgc
cgggaccaaggacacgacggcggtgtttgaggacaagatgcgctacctgaactccctgaagagaaagtacggcaagcctgt
gcctaagaaaattgagtgaacccccgtcgtcgacatgaagagc
SEQ ID NO: 133
gctcttctgcttcggattccactacatcaagtgggtgaacctggcgggcgcggaggagggcccccgcccgggcggcattgtta
gcaaccactgcagctacctggacatcctgctgcacatgtccgactccttccccgcctttgtggcgcgccagtcgacggccaagc
tgccctttatcggcatcatcaggtgcgtgaaagcgggggctgctgtggccgtggtgggcagggttgcgaaggggggcaggcg
toggcgtgcagtgtgagcggacattgatgccgtc+tttgccggtcaggagagctcgaaatcagagccagcctggtcatgggat
cacagagctcaccaccactcgtccacctcgcctgcgccttgcagccaaatcatgagctgcctctacgtgaaccgcgaccgctc
ggggcccaaccacgtgggcgtggccgatctggtgaagcagcgcatgcaggacgaggccgaggggaggaccccgcccgagt
accgaccgctgctcctcttccccgaggtgggctttcgaggcaccgtttgtgcttgaaactgtgggcacgcgtgccccgacgcgc
ctctggcgcctgcttcgcatccattcgcctctcaaccccgtctctcctttcctccatcgccagggcaccacctccaacggcgacta
cctgcttcccttcaagaccggcgccttcctggccggggtgcccgtccagcccgtggtacc
SEQ ID NO: 134
gagctccgtcctccactaccacagggtatggtggtgtggggtcgagcgtgttgaagcgcggaaggggatgctgtcaagttt
tggagctgaaaatggtgcccgcgaggatccagcgcgccccactcacccttgctgccatcgctccccacccttttccccagggaa
ccctgtggcccacgtgggagacgattccggccaagtggcacatcttcctgatgctctgccacccccgccacaaagtgaccgtg
atgaaggtacgaacaagggtcgggccccgattctggatatcacgtctggggtgtgtttctcgcgcacgcgtcccccgatgcgct
gcacagtctccctcacaccctcacccctaacgctcgcagttgcccgtgtacgtccccaatgaggaggaaaaggccgaccccaa
gctgtacgcccaaaatgttcgcaaagccatggtgcgtcgtcgggaaccgttcaagtttgcttgcgggtgggcggggcggctctagc
gaattggcgcattggccctcaccgaggcagcacatcggacaccaatcgtcacccggcgagcaattccgccccctctgtcttctc
gcagatggaggtcgccgggaccaaggacacgacggcggtgtttgaggacaagatgcgctacctgaactccctgaagagaaa
gtacggcaagcctgtgcctaagaaaattgagtgaacccccgtcgtcgaccagaagagc
SEQ ID NO: 135

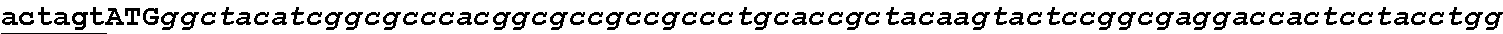

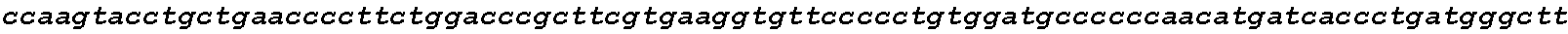

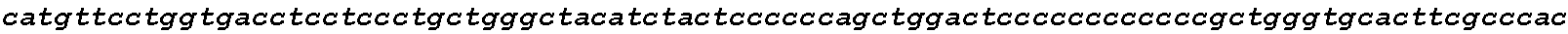

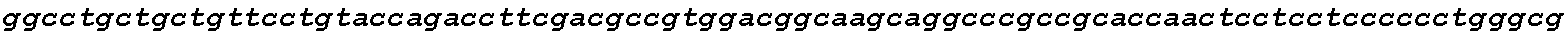

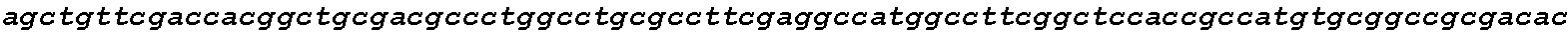

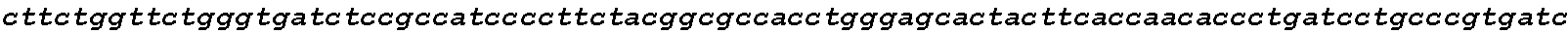

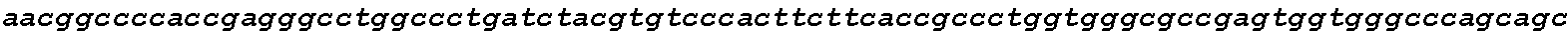

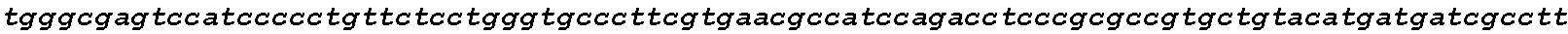

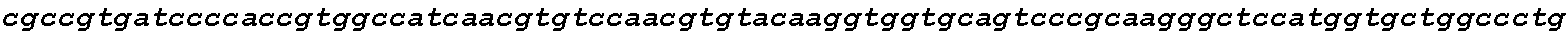

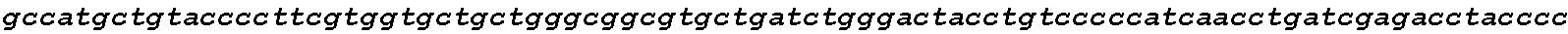

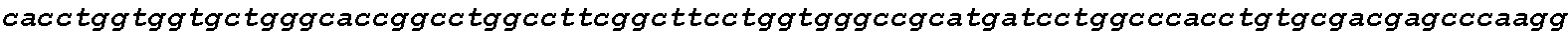

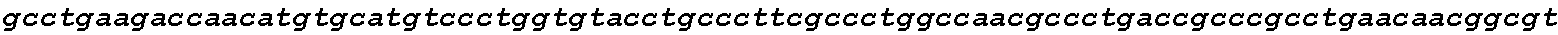

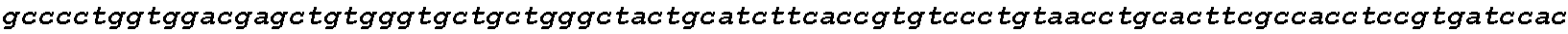

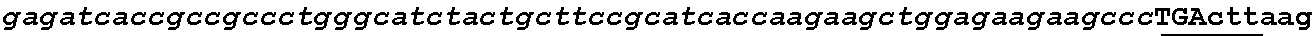

SEQ ID NO: 136

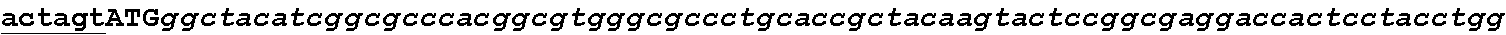

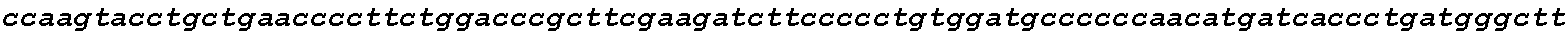

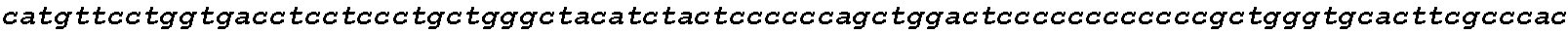

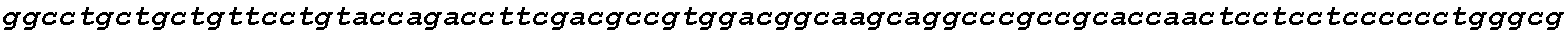

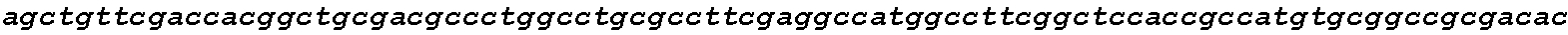

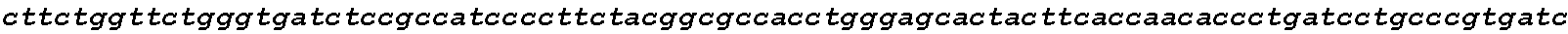

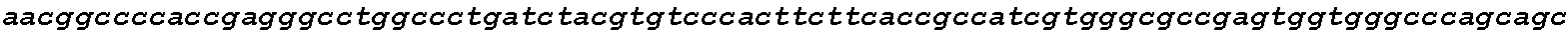

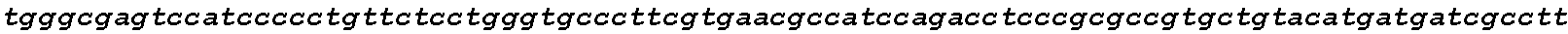

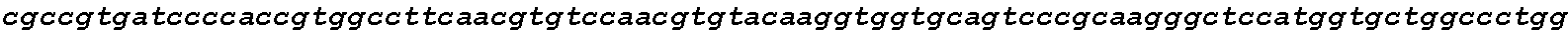

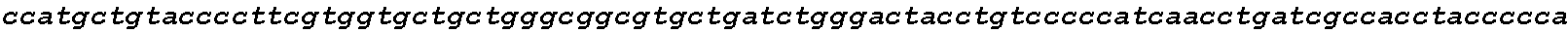

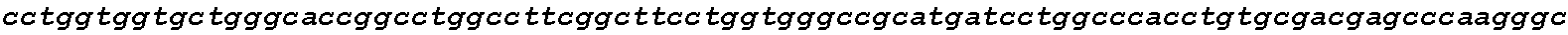

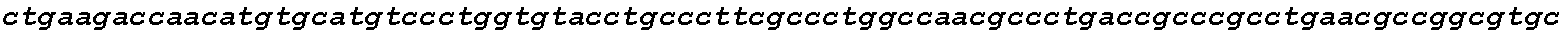

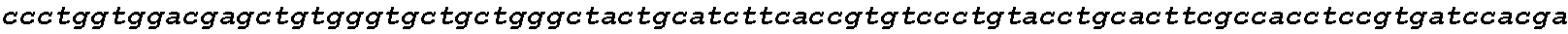

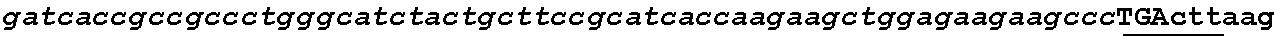

SEQ ID NO: 137
gctcttctgcttcggattccactacatcaagtaagtgaacctggcgggcgcggaggagggcccccgcccgggcggcattgtta
gcaaccattgcagctacctggacatcctgctgcacatgtccgattccttccccgcctttgtggcgcgccagtcgacggccaagc
tgccctttatcggcatcatcaggtgcgtgaaagtgggggctgctgtggtcgtggtgggcggggtcacaaatgaggacattgat
gctgtcgtttgccgatcaggggagctcgaaagtaagtgcagcctggtcatgggatcacaaatctcaccaccactcgtccacctt
gcctgggccttgcagccaaattatgagctgcctctacgtgaaccgcgaccgctcggggcccaaccacgtgggtgtggccgacc
tggtgaagcagcgcatgcaggacgaggccgaggggaagaccccgcccgagtaccggccgctgctcctcttccccgaggtgg
gcttttgagacactgtttgtgcttgaaactgtggacgcgcgtgccctgacgcgcctccggcgcctgtctcgcatccattcgcctct
caaccccatctcaccttttctccatcgccagggcaccacctccaacggcgactacctgcttcccttcaagaccggcgccttcctg
gccggggtgcccgtccagcccgtggtaccgcggtgagaatcgaaaatgcatcgtttctaggttcggagacggtcaattccctgctcc
ggcgaatctgtcggtcaagctggccagtggacaatgttgctatggcagcccgcgcacatgggcctcccgacgcggccatcaggagc
ccaaacagcgtgtcagggtatgtgaaactcaagaggtccclgctgggcactccggccccactccgggggcgggacgccaggcattc
gcggtcggtcccgcgcgacgagcgaaatgatgattcggttacgagaccaggacgtcgtcgaggtcgagaggcagcctcggacacg
tctcgctagggcaacgccccgagtccccgcgagggccgtaaacattgtttctgggtgtcggagtgggcattttgggcccgatccaatc
gcctcatgccgctctcgtctggtcctcacgttcgcgtacggcctggatcccggaaagggcggatgcacgtggtgttgccccgccattg
gcgcccacgtttcaaagtccccggccagaaatgcacaggaccggcccggctcgcacaggccatgctgaacgcccagatttcgaca
gcaacaccatctagaataatcgcaaccatccgcgttttgaacgaaacgaaacggcgctgtttagcatgtttccgacatcgtgggggcc
aagcatgctccggggggaggaaagcgtggcacagcggtagcccattctgtgccacacgccgacgaggaccaatccccggcatca

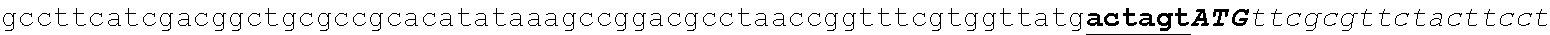

gacggcctgcatctccctgaagggcgtgttcggcgtctccccctcctacaacggcctgggcctgacgccccagatgggctgggac
aactggaacacgttcgcctgcgacgtctccgagcagctgctgctggacacggccgaccgcatctccgacctgggcctgaaggac
atgggctacaagtacatcatcctggacgactgctggtcctccggccgcgactccgacggcttcctggtcgccgacgagcagaagt
tccccaacggcatgggccacgtcgccgaccacctgcacaacaactccttccttcctgttcggcatgtactcctccgcgggcgagtacac
gtgcgccggctaccccggctccctgggccgcgaggaggaggacgcccagttcttcgcgaacaaccgcgtggactacctgaagt
acgacaactgctacaacaagggccagttcggcacgcccgagatctcctaccaccgctacaaggccatgtccgacgccctgaac
aagacgggccgccccatcttctactccctgtgcaactggggccaggacctgaccttctactggggctccggcatcgcgaactcctg
gcgcatgtccggcgacgtcacggcggagttcacgcgccccgactcccgctgcccctgcgacggcgacgagtacgactgcaagt
acgccggcttccactgctccatcatgaacacatcctgaacaaggccgcccccatgggccagaacgcgggcgtcggcggctggaac
gacctggacaacctggaggtcggcgtcggcaacctgacggacgacgaggagaaggcgcacttctccatgtgggccatggtgaa
gtcccccctgatcatcggcgcgaacgtgaacaacctgaaggcctcctcctactccatctactcccaggcgtccgtcatcgccatca
accaggactccaacggcatccccgccacgcgcgtctggcgctactacgtgtccgacacggacgagtacggccagggcgagatc
cagatgtggtccggccccctggacaacggcgaccaggtcgtggcgctgctgaacggcggctccgtgtcccgccccatgaacacg
accctggaggagatcttcttcgactccaacctgggctccaagaagctgacctccacctgggacatctacgacctgtgggcgaacc
gcgtcgacaactccacggcgtccgccatgggccgcaacaagaccgccaccggcatcctgtacaacgccaccgagcagtcc
tacaaggacggcctgtccaagaacgacacccgcctgacggccagaagatcggctccctgtcccccaacgcgatcctgaacacg

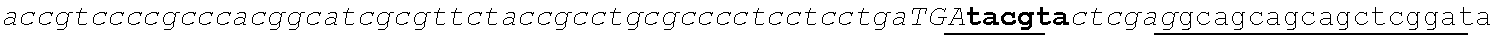

gtatcgacacactctggacgctggtcgtgtgatggactgttgccgccacacttgctgccttgacctgtgaatatccctgccgcttttatcaa
acagcctcagtgtgatgatcagtgtgtacgcgcattgcgagagctagctgatgtgctatttgcgaataccacccccagcatccccttc
cctcgatcatatcgcttgcatcccaaccgcaacttatctacgctgtcctgctatccctcagcgctgctcctgctcctgctcactgcccctcg
cacagccaggtagggctccgcctgtattctcctggtactgcaacctgtaaaccagcactgcaatgctgatgcacgggaagtagtggg
atgggaacacaaatggaaagctgtagaattcctggctcgggcctcgtgctggcactccctcccatgccgacaacctactgctgtcacc
acgacccacgatgcaacgcgacacgacccggtgggactgatcggacactgcacctgcatgcaattgtcacaagcgcatactccaat
cgtatccgtttgatttctgtgaaaactcgctcgaccgcccgcgtcccgcaggcagcgatgacgtgtgcgtgacctgggtgtttcgtcga
aaggccagcaaccccaaatcgcaggcgatccggagattgggatctgatccgagcaggaccagatcccccacgatgcggcacggg
aactgcatcgactcggcgcggaacccagattcgtaaatgccagattggtgtccgataccttgatttgccatcagcgaaacaagacttca
gcagcgagcgtataggcgggcgtgctaccagggagcatacattgcccatactgtctggaccgattaccggcgcagagggtgagtt
gatggggaggcaggcatcgaaacgcgcgtgcatggtgtgtgtgtctgattcggctgcacaatttcaatagtcggatgggcgacggta
gaattgggtgttgcgctcgcgtgcatgcctcgccccgtcgggtgtcatgaccgggactggaatcccccctcgcgaccctcctgctaac

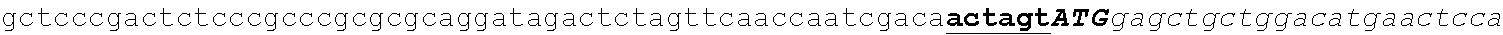

tggccgcctccatcggcgtgtccgtggccgtgctgcgcttcctgctgtgcttcgtggccaccatccccatctccttcctgtggcgcttca
tcccctcccgcctgggcaagcacatctactccgccgcctccggcgccttcctgtcctacctgtcatcggcttctcctccaacctgcac
ttcctggtgcccatgaccatcggctacgcctccatggccatctaccgccccctgtccggcttcatcaccttcttcctgggcttcgcctac
ctgatcggctgccacgtgttctacatgtccggcgacgcctggaaggagggcggcatcgactccaccggcgccctgatggtgctga
ccctgaaggtgatctcctgctccatcaactacaacgacggcatgctgaaggaggagggcctgcgcgaggcccagaagaagaa
ccgcctgatccagatgccctccctgatcgagtacttcggctactgcctgtgctgcggctcccacttcgccggccccgtgacgagatg
aaggactacctggagtggaccgaggagaagggcatctgggccgtgtccgagaagggcaagcgcccctccccctacggcgcca
tgatccgcgccgtgaccaggccgccatctgcatggccctgtacctgtacctggtgccccagttccccctgacccgcttcaccgagc
ccgtgtaccaggagtggggcttcctgaagcgcttcggctaccagtacatggccggcttcaccgcccgctggaagtactacttcatct
ggtccatctccgaggcctccatcatcatctccggcctgggcttctccggctggaccgacgagacccagaccaaggccaagtggg
accgcgccaagaacgtggacatcctgggcgtggagctggccaagtccgccgtgcagatccccctgttctggaacatccaggtgtc
cacctggctgcgccactacgtgtacgagcgcatcgtgaagcccggcaagaaggccggcttcttccagctgctggccacccagac
cgtgtccgccgtgtggcacggcctgtaccccggctacatcatcttcttcgtgcagtccgccctgatgatcgacggctccaaggccat
ctaccgctggcagcaggccatcccccccaagatggccatgctgcgcaacgtgctggtgctgatcaacttcctgtacaccgtggtgg
tgctgaactactcctccgtgggcttcatggtgctgtccctgcacgagaccctggtggccttcaagtccgtgtactacatcggcaccgt

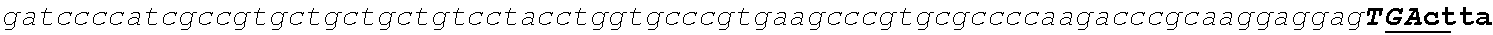

aggcagcagcagctcggatagtatcgacacactctggacgctggtcgtgtgatggactgagccgccacacttgctgccttgacctgtg
aatatccctgccgcattatcaaacagcctcagtgtgatgatcagtgtgtacgcgcattgcgagagctagctgcttgtgctatttgcgaat
accacccccagcatccccttccctcgtttcatatcgcttgcatcccaaccgcaacttatctacgctgtcctgctatccctcagcgctgctcc
tgctcctgctcactgcccctcgcacagccttggtttgggctccgcctgtattctcctggtactgcaacctgtaaaccagcactgcaatgct
gatgcacgggaagtagtgggatgggaacacaaatggaaagcttaattaagagctccgtcctccactaccacagggtatggtcgtgt
ggggtcgagcgtgttgaagcgcagaaggggatgcgccgtcaagatcaggagctaaaaatggtgccagcgaggatccagcg
ctctcactcttgctgccatcgctcccacccttttccccaggggaccctgtggcccacgtgggagacgattccggccaagtggcac
atcttcctgatgctctgccacccccgccacaaagtgaccgtgatgaaggttaggacaagggtcgggacccgattctggatatg
acctctgaggtgtgtttctcgcgcaagcgtcccccaattcgttacaccacatccctcacaccctcgcccctgacactcgcagttg
cccgtgtacgtccccaatgaggaggaaaaggccgaccccaagctgtacgcccaaaacgtccgcaaagccatggtgcgtcgg
gaaccgtcaaagtttgcttgcgggtgggcggggcggctctagcgaattggctcattggccctcaccgaggcagcacatcggac
accagtcgccacccggcttgcatcttcgccccctttcttctcgcagatggaggtcgccgggaccaaggacacgacggcggtgtt
tgaggacaagatgcgctacctgaactccctgaagagaaagtacggcaagcctgtgcctaagaaaattgagtgaacccccgtc
gtcgaccagaagagc
SEQ ID NO: 138

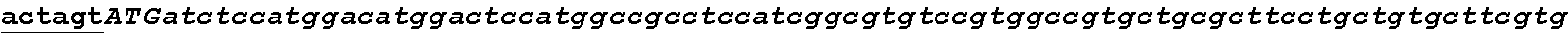

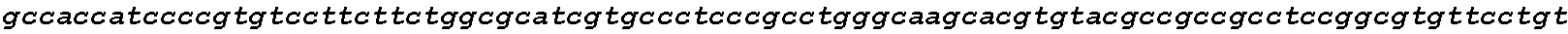

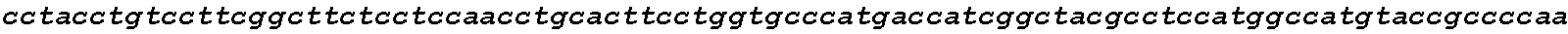

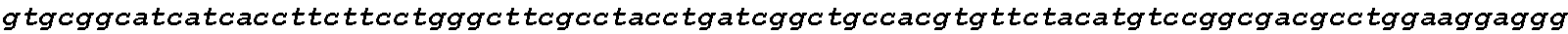

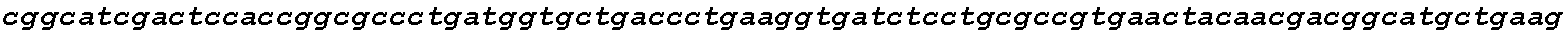

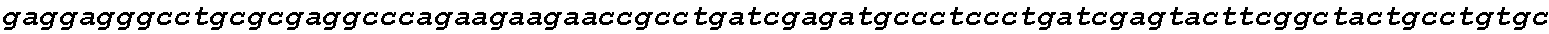

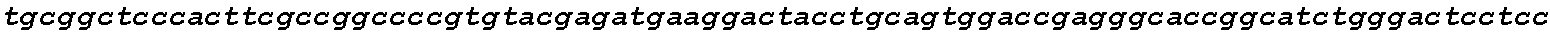

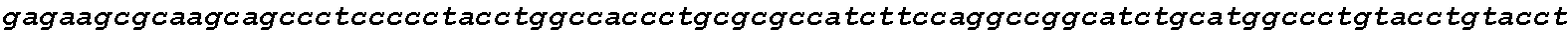

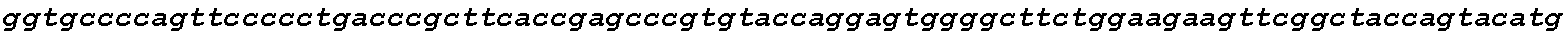

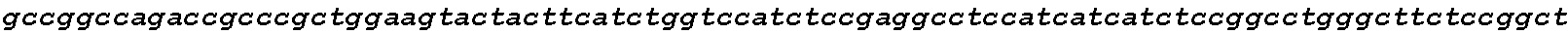

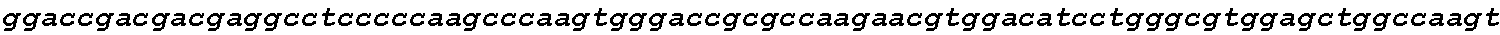

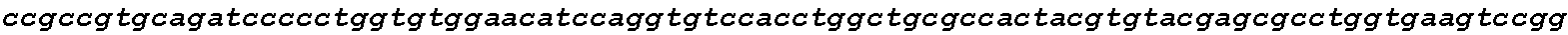

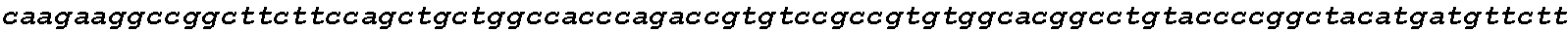

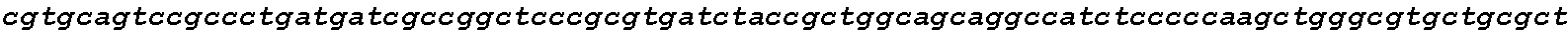

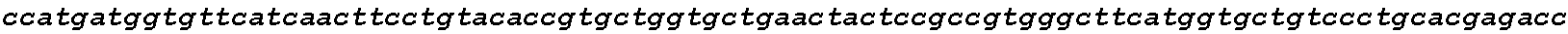

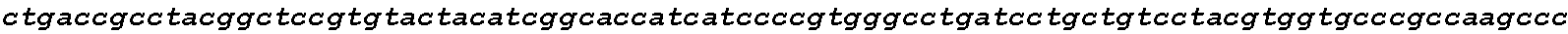

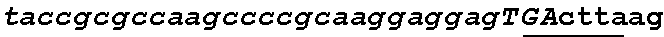

SEQ ID NO: 139
gctcttcgcgaaggtcattttccagaacaacgaccatggcttgtcttagcgatcgctcgaatgactgctagtgagtcgtacgctcgacccagt
cgctcgcaggagaacgcggcaactgccgagcttcggcttgccagtcgtgactcgtatgtgatcaggaatcattggcattggtagcattata
attcggcttccgcgctgtttatgggcatggcaatgtctcatgcagtcgaccttagtcaaccaattctgggtggccagctccgggcgaccggg
ctccgtgtcgccgggcaccacctcctgccatgagtaacagggccgccctctcctcccgacgttggcccactgaataccgtgtcttggggccc
tacatgatgggctgcctagtcgggcgggacgcgcaactgcccgcgcaatctgggattctgggacgtggtctgaatcctccaggcgggtttccccgaga
aagaaagggtgccgatttcaaagcagagccatgtgccgggccctgtggcctgtgttggcctgtgttggcgcctatgtagtcaccccccctcacccaattgtc
gccagtttgcgcaatccataaactcaaaactgcagcttctgagctgcgctgttcaagaacacctctggggtttgctcacccgcgaggtcgac

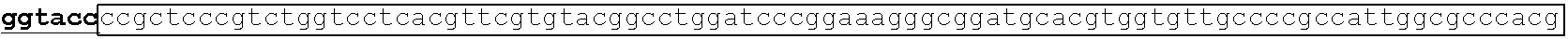

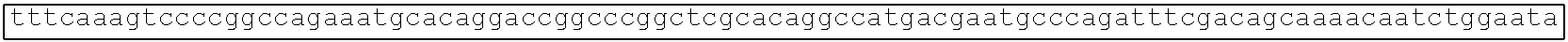

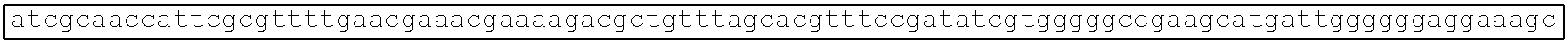

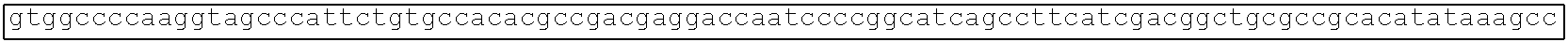

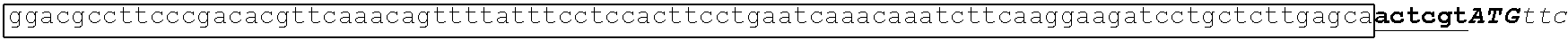

gcgttctacttcctgacggcctgcatctccctgaagggcgtgttcggcgtctccccctcctacaacggcctgggcctgacgccccagatgggctg
ggacaactggaacacgttcgcctgcgacgtctccgagcagctgctgctggacacggccgaccgcatctccgacctgggcctgaaggacatg
ggctacaagtacatcatcctggacgactgctggtcctccggccgcgactccgacggcttcctggtcgccgacgagcagaagttccccaacggc
atgggccacgtcgccgaccacctgcacaacaactccttcctgttcggcatgtactcctccgcgggcgagtacacgtgcgccggctaccccggc
tccctgggccgcgaggaggaggacgcccagttcttcgcgaacaaccgcgtggactacctgaagtacgacaactgctacaacaagggccagt
tcggcacgcccgagatctcctaccaccgctacaaggccatgtccgacgccctgaacaagacgggccgccccatcttctactccctgtgcaact
ggggccaggacctgaccttctactggggctccggcatcgcgaactcctggcgcatgtccggcgacgtcacggcggagttcacgcgccccgac
tcccgctgcccctgcgacggcgacgagtacgactgcaagtacgccggcttccactgctccatcatgaacatcctgaacaaggccgcccccat
gggccagaacgcgggcgtcggcggctggaacgacctggacaacctggaggtcggcgtcggcaacctgacggacgacgaggagaaggc
gcacttctccatgtgggccatggtgaagtcccccctgatcatcggcgcgaacgtgaacaacctgaaggcctcctcctactccatctactcccag
gcgtccgtcatcgccatcaaccaggactccaacggcatccccgccacgcgcgtctggcgctactacgtgtccgctcacggctcgagtacggcca
gggcgagatccagatgtggtccggccccctggacaacggcgaccaggtcgtggcgctgctgctgaacggcggctccgtgtcccgccccatgaac
acgaccctggaggagatcttcttcgactccaacctgggctccaagaagctgacctccacctgggacatctacgacctgtgggcgaaccgcgtc
gacaactccacggcgtccgccatcctgggccgcaacaagaccgccaccggcatcctgtacaacgccaccgagcagtcctacaaggacggc
ctgtccaagaacgacacccgcctgncggccagaagatcggctccctgtcccccaacgcgatcctgaacacgaccgtccccgcccacggcat

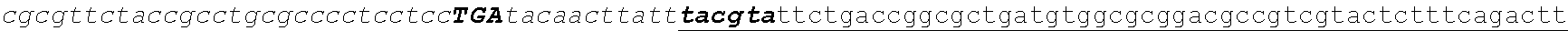

tactcttgaggaattgaacctttctcgcttgctggcatgtaaacattggcgcaattaattgtgtgatgaagaaagggtggcacaagatggatcgcgaat
gtacgagatcgacaacgatggtgattgttatgaggggceaaacctggctcaatcttgtcgcatgtccggcgcaatgtgatccagcggcgtgactctc
gcaacctggtagtgtgtgcgcaccgggtcgctttgattaaaactgatcgcattgccatcccgtcaactcacaagcctactctagctcccattgcgcact
cgggcgcccggctcgatcaatgttctgagcggagggcgaagcgtcaggaaatcgtctcggcagctggaagcgcatggaatgcggagcggagat
cgaatcaggatcccgcgtctcgaacagagcgcgcagaggaacgctgaaggtctcgcctctgtcgcacctcagcgcggcatacaccacaataacc
acctgacgaatgcgcttggttcttcgtccattagcgaagcgtccggttcacacacgtgccacgttggcgaggtggcaggtgacaatgatcggtgga

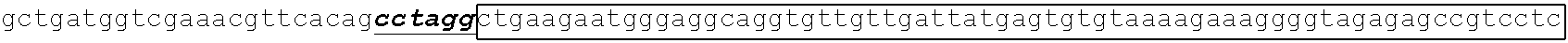

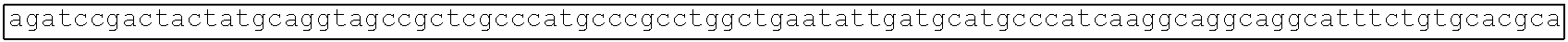

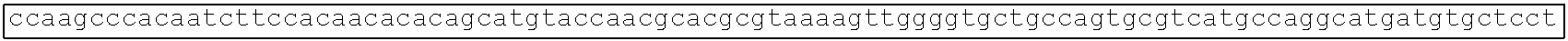

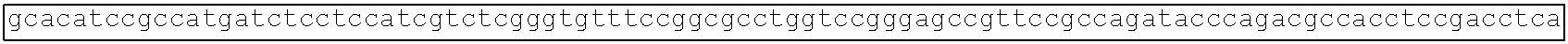

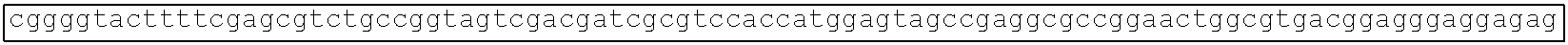

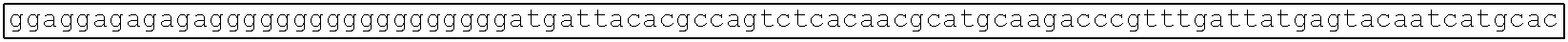

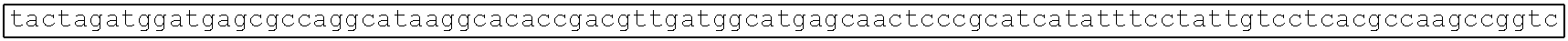

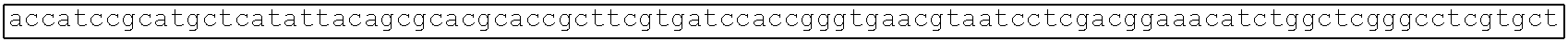

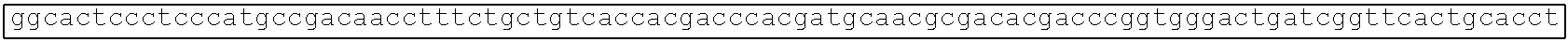

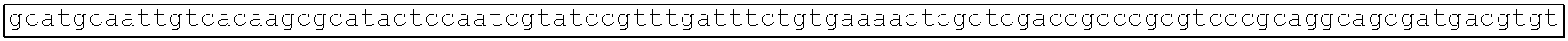

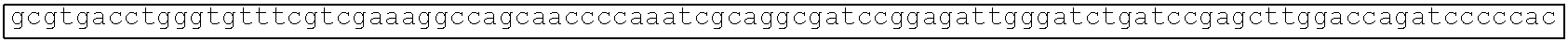

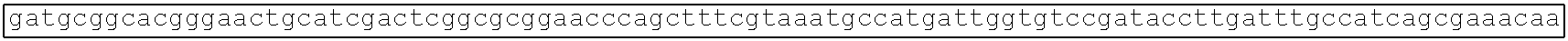

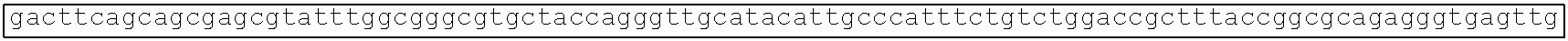

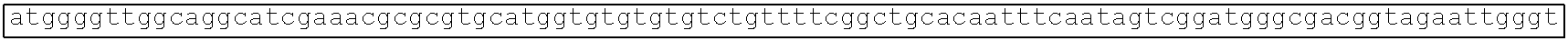

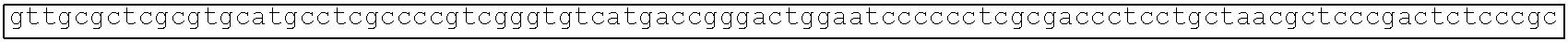

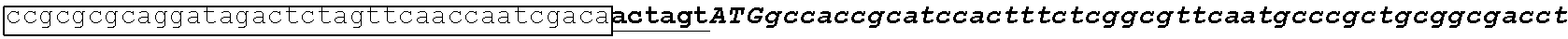

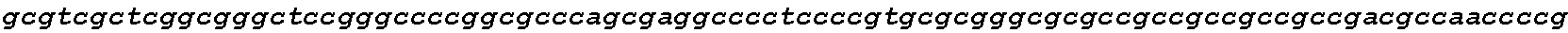

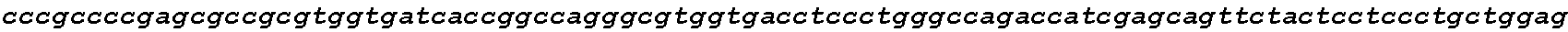

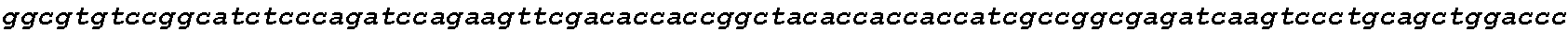

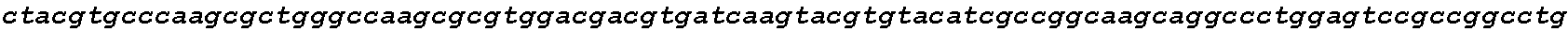

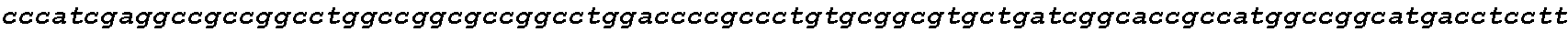

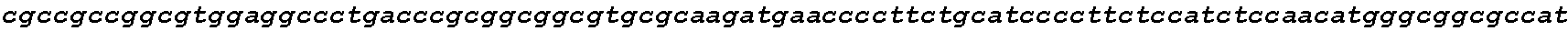

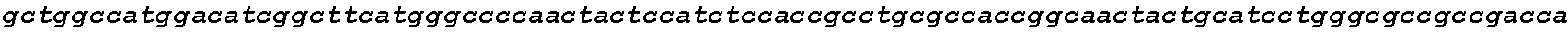

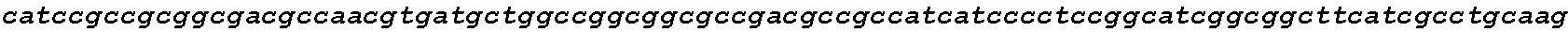

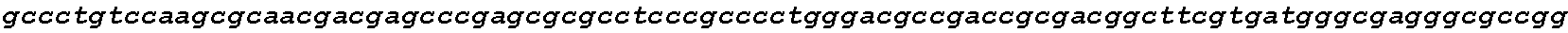

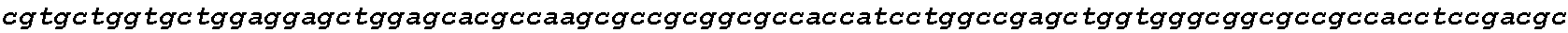

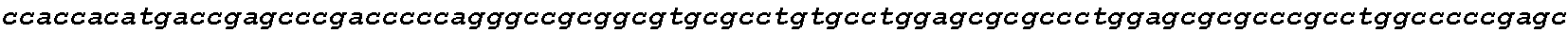

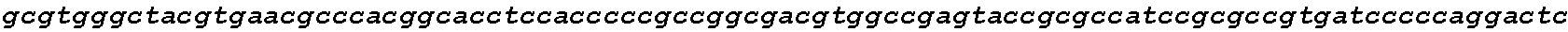

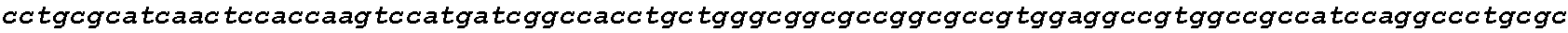

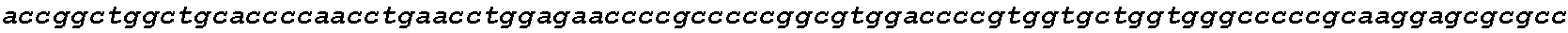

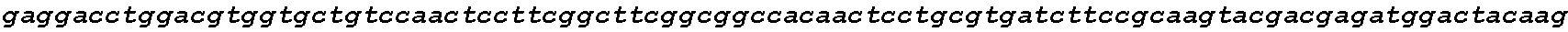

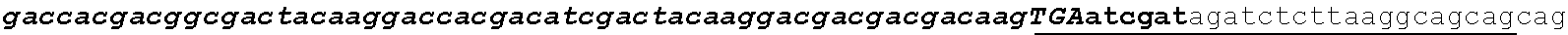

ctcggatagtatcgacacactctggacgctggtcgtgtgatggactgttgccgccacacttgctgccttgacctgtgaatatccctgccgcttttatcaa
acagcctcagtgtgtttgatcttgtgtgtacgcgcttttgcgagttgctagctgcttgtgctatttgcgaataccacccccagcatccccttccctcgtttc
atatcgcttgcatcccaaccgcaacttatctacgctgtcctgctatccctcagcgctgctcctgctcctgctcactgcccctcgcacagccttggtttgg
gctccgcctgtattctcctggtactgcaacctgtaaaccagcactgcaatgctgatgcacgggaagtagtgggatgggaacacaaatggagaattc

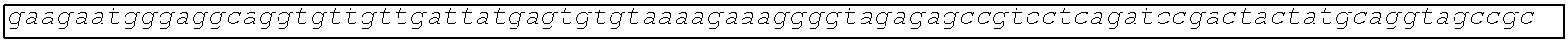

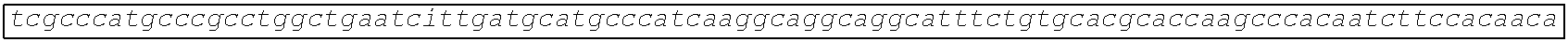

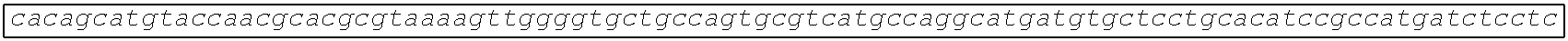

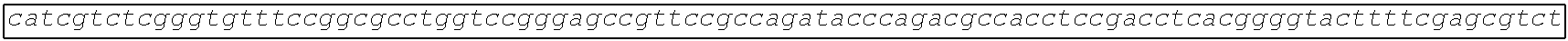

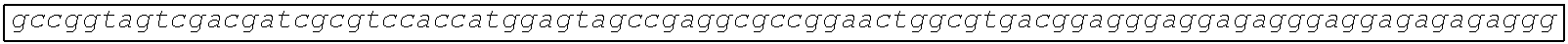

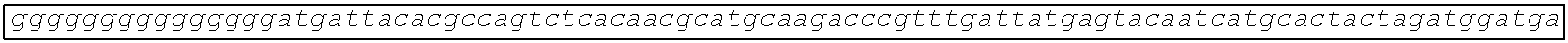

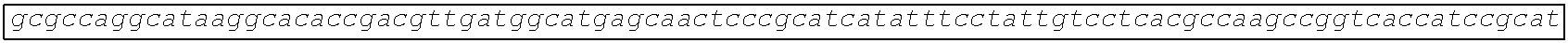

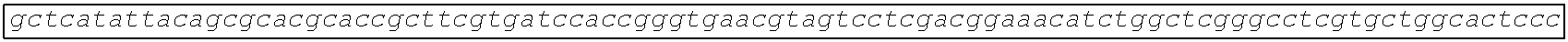

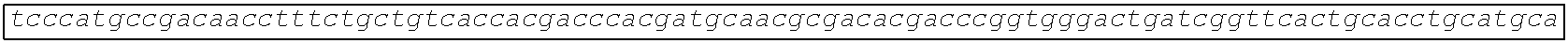

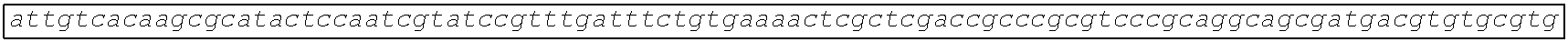

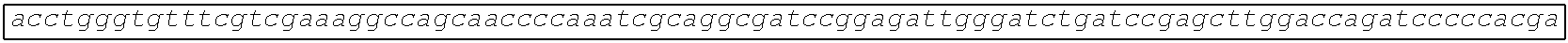

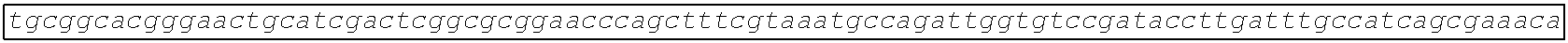

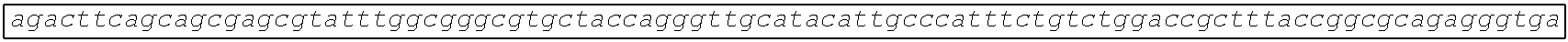

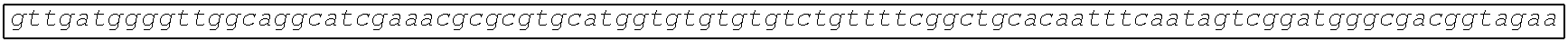

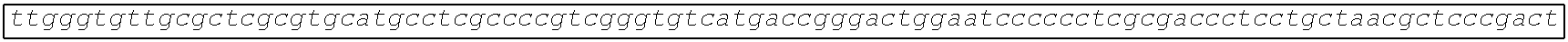

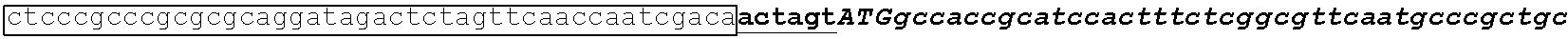

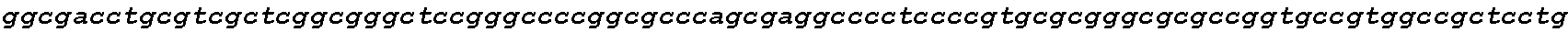

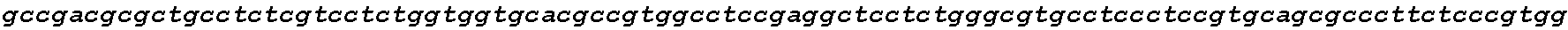

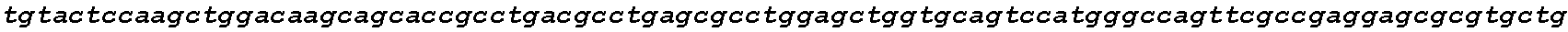

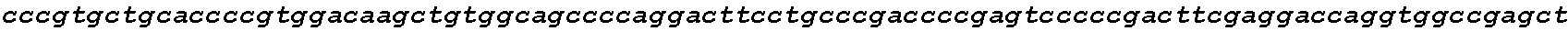

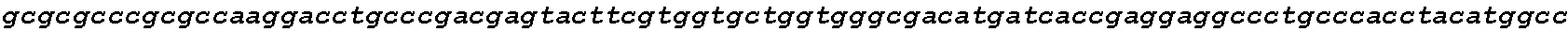

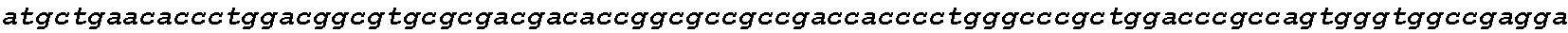

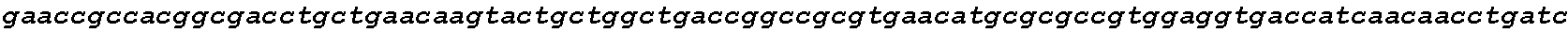

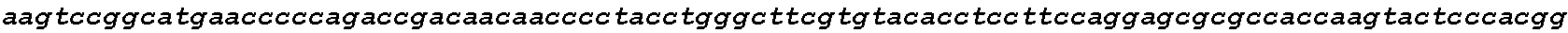

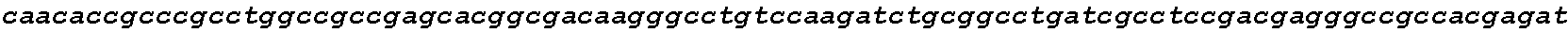

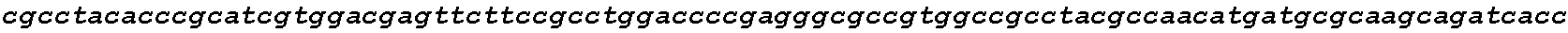

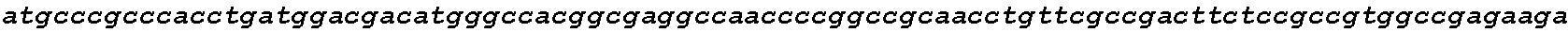

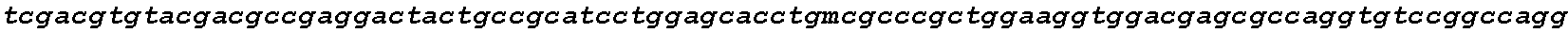

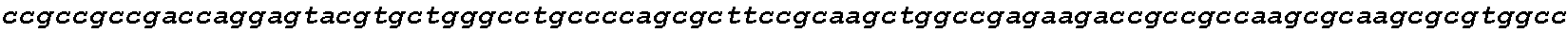

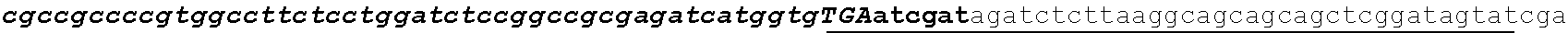

cacactctggacgctggtcgtgtgatggactgttgccgccacacttgctgccttgacctgtgaatatccctgccgcttttatcaaacagcctcagtgtgt
ttgatcttgtgtgtacgcgcttttgcgagttgctagctgcttgtgctatttgcgaataccacccccagcatccccttccctcgtttcatatcgcttgcatccc
aaccgcaacttatctacgctgtcctgctatccctcagcgctgctcctgctcctgctcactgcccctcgcacagccttggtttgggctccgcctgtattct
cctggtactgcaacctgtaaaccagcactgcaatgctgatgcacgggaagtagtgggatgggaacacaaatggaaagcttaattaagagctcctc
actcagcgcgcctgcgcggggatgcggaacgccgccgccgccttgtcttttgcacgcgcgactccgtcgcttcgcgggtggcacccccatt
gaaaaaaacctcaattctgtttgtggaagacacggtgtacccccaaccacccacctgcacctctattattggtattattgacgcgggagcgg
gcgttgtactctacaacgtagcgtctctggttttcagctggctcccaccattgtaaattcttgctaaaatagtgcgtggttatgtgagaggtat
ggtgtaacagggcgtcagtcatgttggttttcgtgctgatctcgggcacaaggcgtcgtcgacgtgacgtgcccgtgatgagagcaatacc
gcgctcaaagccgacgcatggcctttactccgcactccaaacgactgtcgctcgtatttttcggatatctattttttaagagcgagcacagcg
ccgggcatgggcctgaaaggcctcgcggccgtgctcgtggtgggggccgcgagcgcgtggggcatcgcggcagtgcaccaggcgcaga
cggaggaacgcatggtgagtgcgcatcacaagatgcatgtcttgttgtctgtactataatgctagagcatcaccaggggcttagtcatcgca
cctgctttggtcattacagaaattgcacaagggcgtcctccgggatgaggagatgtaccagctcaagctggagcggcttcgagccaagca
ggagcgcggcgcatgacgacctacccacatgcgaagagc
SEQ ID NO: 140
gctcttcacccaactcagataataccaatacccctccttctcctcctcatccattcagtacccccccccttctcttcccaaagcagcaagcgcg
tggcttacagaagaacaatcggcttccgccaaagtcgccgagcactgcccgacggcggcgcgcccagcagcccgcttggccacacaggc
aacgaatacattcaatagggggcctcgcagaatggaaggagcggtaaagggtacaggagcactgcgcacaaggggcctgtgcaggag
tgactgactgggcgggcagacggcgcaccgcgggcgcaggcaagcagggaagattgaagcggcagggaggaggatgctgattgagg
ggggcatcgcagtctctcttggacccgggataaggaagcaaatattcggccggttgggttgtgtgtgtgcacgttttcttcttcagagtcgtg

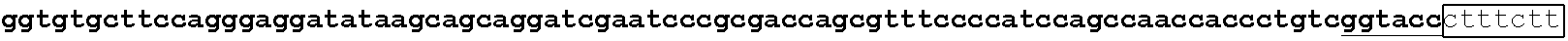

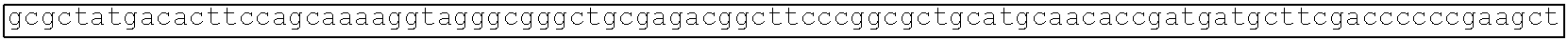

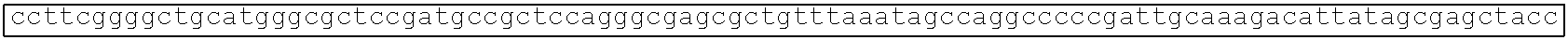

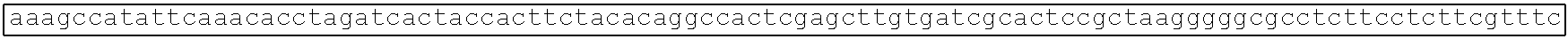

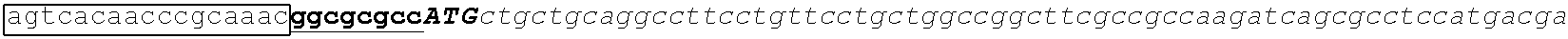

acgagacgtccgaccgccccctggtgcacttcacccccaacaagggctggatgaacgaccccaacggcctgtggtacgacgagaaggacg
ccaagtggcacctgtacttccagtacaacccgaacgacaccgtctgggggacgcccttgttctggggccacgccacgtccgacgacctgacc
aactgggaggaccagcccatcgccatcgccccgaagcgcaacgactccggcgccttctccggctccatggtggtggactacaacaacacct
ccggcttcttcaacgacaccatcgacccgcgccagcgctgcgtggccatctggacctacaacaccccggagtccgaggagcagtacatctcc
tacagcctggacggcggctacaccttcaccgagtaccagaagaaccccgtgctggccgccaactccacccagttccgcgacccgaaggtctt
ctggtacgagccctcccagaagtggatcatgaccgcggccaagtcccaggactacaagatcgagatctactcctccgacgacctgaagtcct
ggaagctggagtccgcgttcgccaacgagggcttcctcggctaccagtacgagtgccccggcctgatcgaggtccccaccgagcaggaccc
cagcaagtcctactgggtgatgttcatctccatcaaccccggcgccccggccggcggctccttcaaccagtacttcgtcggcagcttcaacggc
acccacttcgaggccttcgacaaccagtcccgcgtggtggacttcggcaaggactactacgccctgcagaccttcttcaacaccgacccgacc
tacgggagcgccctgggcatcgcgtgggcctccaactgggagtactccgccttcgtgcccaccaacccctggcgctcctccatgtccctcgtgc
gcaagttctccctcaacaccgagtaccaggccaacccggagacggagctgatcaacctgaaggccgagccgatcctgaacatcagcaacg
ccggcccctggagccggttcgccaccaacaccacgttgacgaaggccaacagctacaacgtcgacctgtccaacagcaccggcaccctgg
agttcgagctggtgtacgccgtcaacaccacccagacgatctccaagtccgtgttcgcggacctctccctctggttcaagggcctggaggaccc
cgaggagtacctccgcatgggcttcgaggtgtccgcgtcctccttcttcctggaccgcgggaacagcaaggtgaagttcgtgaaggagaaccc
ctacttcaccaaccgcatgagcgtgaacaaccagcccttcaagagcgagaacgacctgtcctactacaaggtgtacggcttgctggaccaga
acatcctggagctgtacttcaacgacggcgacgtcgtgtccaccaacacctacttcatgaccaccgggaacgccctgggctccgtgaacatga

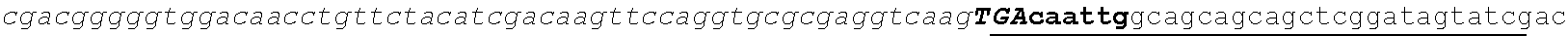

acactctggacgctggtcgtgtgatggactgttgccgccacacttgctgccttgacctgtgaatatccctgccgcttttatcaaacagcctcagtgtgttt
gatcttgtgtgtacgcgcttttgcgagttgctagctgcttgtgctatttgcgaataccacccccagcatccccttccctcgtttcatatcgcttgcatccca
accgcaacttatctacgctgtcctgctatccctcagcgctgctcctgctcctgctcactgcccctcgcacagccaggtagggctccgcctgtaactc
ctggtactgcaacctgtaaaccagcactgcaatgctgatgcacgggaagtagtgggatgggaacacaaatggaggatcccgcgtctcgaacaga
gcgcgcagaggaacgctgaaggtctcgcctctgtcgcacctcagcgcggcatacaccacaataaccacctgacgaatgcgcaggacacgtcca
ttagcgaagcgtccggttcacacacgtgccacgttggcgaggtggcaggtgacaatgatcggtggagctgatggtcgaaacgttcacagcctagg

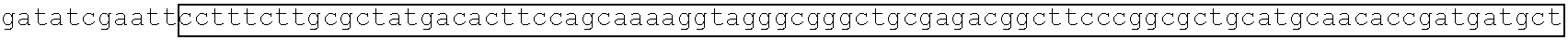

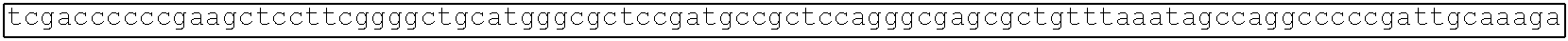

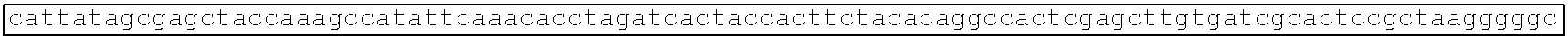

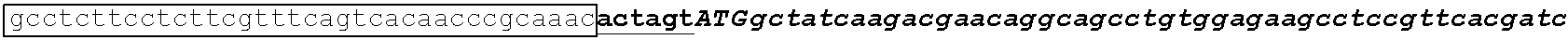

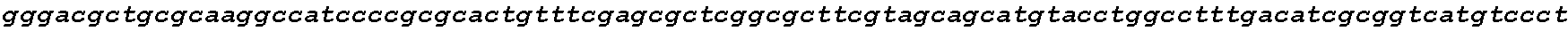

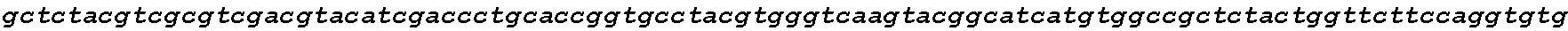

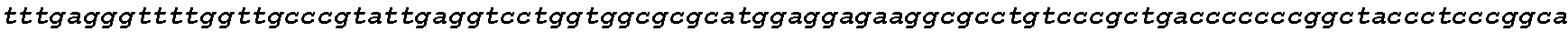

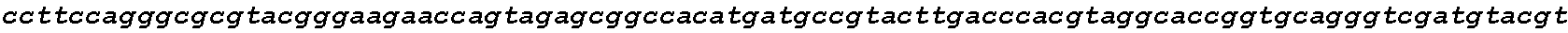

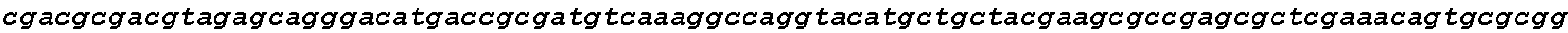

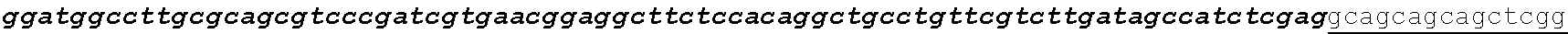

atagtatcgacacactctggacgctggtcgtgtgatggactgagccgccacacagctgccagacctgtgaatatccctgccgcattatcaaacagc
ctcagtgtgtttgatcttgtgtgtacgcgcttttgcgagttgctagctgcttgtgctatttgcgaataccacccccagcatccccttccctcgtttcatatcg
cagcatcccaaccgcaacttatctacgctgtcctgctatccctcagcgctgctcctgctcctgctcactgcccctcgcacagccaggtagggctccg
cctgtaactcctggtactgcaacctgtaaaccagcactgcaatgctgatgcacgggaagtagtgggatgggaacacaaatggaaagctgtagagc
tcagattccagaaggagagctccagagccatcattctcagcctcgataacctccaaagccgctctaattgtggagggggacgaaccgaatgctg
cgtgaacgggaaggaggaggagaaagagtgagcagggagggattcagaaatgagaaatgagaggtgaaggaacgcatccctatgcc
cttgcaatggacagtgtttctggccaccgccaccaagacttcgtgtcctctgatcatcatgcgattgattacgttgaatgcgacggccggtca
gccccggacctccacgcaccggtgctcctccaggaagatgcgcttgtcctccgccatcttgcagggctcaagctgctcccaaaactcttggg
cgggttccggacggacggctaccgcgggtgcggccctgaccgccactgttcggaagcagcggcgctgcatgggcagcggccgctgcggt
gcgccacggaccgcatgatccaccggaaaagcgcacgcgctggagcgcgcagaggaccacagagaagcggaagagacgccagtact
ggcaagcaggctggtcggtgccatggcgcgctactaccctcgctatgactcgggtcctcggccggctggcggtgctgacaattcgtttagtg
gagcagcgactccattcagctaccagtcgaactcagtggcacagtgactccgctcttc
SEQ ID NO: 141
gctcttcgccgccgccactcctgctcgagcgcgcccgcgcgtgcgccgccagcgccttggccttttcgccgcgctcgtgcgcgtcgctgatgt
ccatcaccaggtccatgaggtctgccttgcgccggctgagccactgcttcgtccgggcggccaagaggagcatgagggaggactcctggt
ccagggtcctgacgtggtcgcggctctgggagcgggccagcatcatctggctctgccgcaccgaggccgcctccaactggtcctccagca
gccgcagtcgccgccgaccctggcagaggaagacaggtgaggggggtatgaattgtacagaacaaccacgagccttgtctaggcagaa
tccctaccagtcatggctttacctggatgacggcctgcgaacagctgtccagcgaccctcgctgccgccgcttctcccgcacgcttctttcca
gcaccgtgatggcgcgagccagcgccgcacgctggcgctgcgcttcgccgatctgaggacagtcggggaactctgatcagtctaaacccc

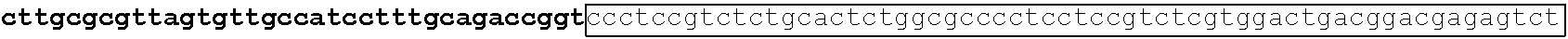

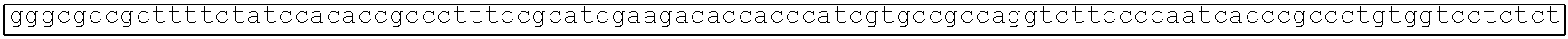

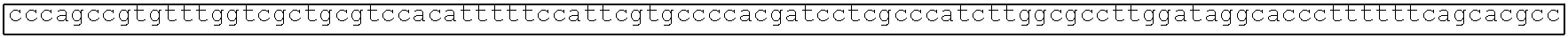

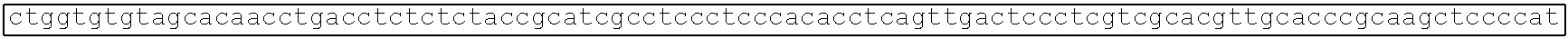

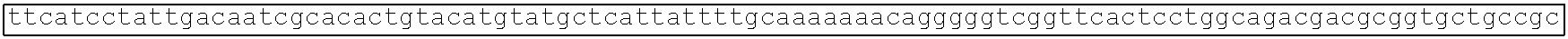

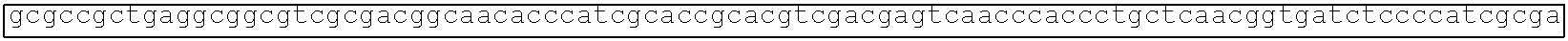

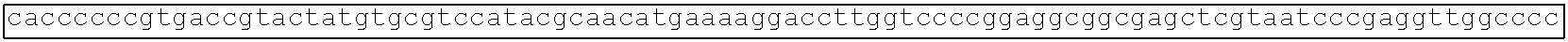

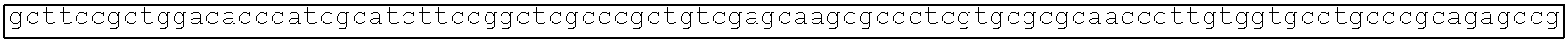

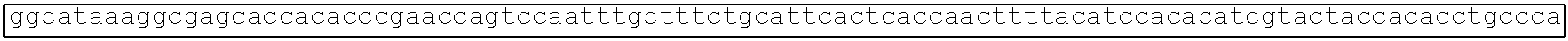

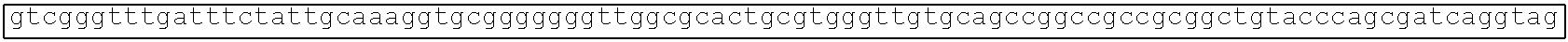

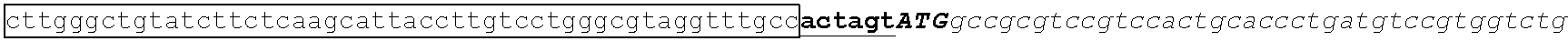

caacaacaagaaccactccgcccgccccaagctgcccaactcctccctgctgcccggcttcgacgtggtggtccaggccgcggccacccgct
tcaagaaggagacgacgaccacccgcgccacgctgacgttcgacccccccacgaccaactccgagcgcgccaagcagcgcaagcacac
catcgacccctcctcccccgacttccagcccatcccctccttcgaggagtgcttccccaagtccacgaaggagcacaaggaggtggtgcacga
ggagtccggccacgtcctgaaggtgccatccgccgcgtgcacctgtccggcggcgagcccgcatcgacaactacgacacgtccggccccc
agaacgtcaacgcccacatcggcctggcgaagctgcgcaaggagtggatcgaccgccgcgagaagctgggcacgccccgctacacgcag
atgtactacgcgaagcagggcatcatcacggaggagatgctgtactgcgcgacgcgcgagaagctggaccccgagttcgtccgctccgagg
tcgcgcggggccgcgccatcatcccctccaacaagaagcacctggagctggagcccatgatcgtgggccgcaagttcctggtgaaggtgaa
cgcgaacatcggcaactccgccgtggcctcctccatcgaggaggaggtctacaaggtgcagtgggccaccatgtggggcgccgacaccatc
atggacctgtccacgggccgccacatccacgagacgcgcgagtggatcctgcgcaactccgcggtccccgtgggcaccgtccccatctacca
ggcgctggagaaggtggacggcatcgcggagaacctgaactgggaggtgttccgcgagacgctgatcgagcaggccgagcagggcgtgg
actacttcacgatccacgcgggcgtgctgctgcgctacatccccctgaccgccaagcgcctgacgggcatcgtgtcccgcggcggctccatcc
acgcgaagtggtgcctggcctaccacaaggagaacttcgcctacgagcactgggacgacatcctggacatctgcaaccagtacgacgtcgc
cctgtccatcggcgacggcctgcgccccggctccatctacgacgccaacgacacggcccagttcgccgagctgctgacccagggcgagctg
acgcgccgcgcgtgggagaaggacgtgcaggtgatgaacgagggccccggccacgtgcccatgcacaagatccccgagaacatgcaga
agcagctggagtggtgcaacgaggcgcccttctacaccctgggccccctgacgaccgacatcgcgcccggctacgaccacatcacctccgc
catcggcgcggccaacatcggcgccctgggcaccgccctgctgtgctacgtgacgcccaaggagcacctgggcctgcccaaccgcgacga
cgtgaaggcgggcgtcatcgcctacaagatcgccgcccacgcggccgacctggccaagcagcacccccacgcccaggcgtgggacgacg
cgctgtccaaggcgcgcttcgagttccgctggatggaccagttcgcgctgtccctggaccccatgacggcgatgtccttccacgacgagacgct
gcccgcggacggcgcgaaggtcgcccacttctgctccatgtgcggccccaagttctgctccatgaagatcacggaggacatccgcaagtacg
ccgaggagaacggctacggctccgccgaggaggccatccgccagggcatggacgccatgtccgaggagttcaacatcgccaagaagacg

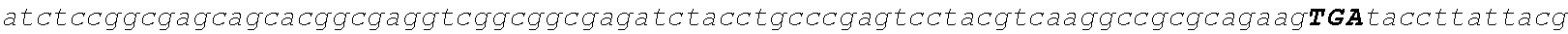

taacagacgaccaggcaggcgtcgggtagggaggtggtggtgatggcgtctcgatgccatcgcacgcatccaacgaccgtatacgcatcgtcca
atgaccgtcggtgtcctctctgcctccgattgtgagatgtctcaggcttggtgcatcctcgggtggccagccacgttgcgcgtcgtgctgcttgcctct
cttgcgcctctgtggtactggaaaatatcatcgaggcccgtttttttgctcccatttcctttccgctacatcttgaaagcaaacgacaaacgaagcagca
agcaaagagcacgaggacggtgaacaagtctgtcacctgtatacatctatttccccgcgggtgcacctactctctctcctgccccggcagagtcagc

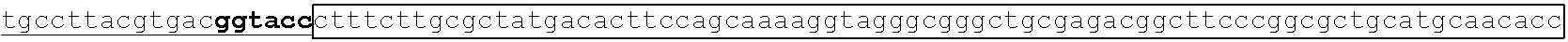

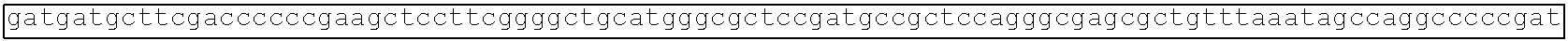

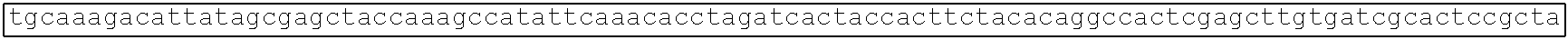

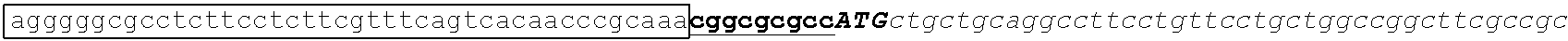

caagatcagcgcctccatgacgaacgagacgtccgaccgccccctggtgcacttcacccccaacaagggctggatgaacgaccccaacgg
cctgtggtacgacgagaaggacgccaagtggcacctgtacttccagtacaacccgaacgacaccgtctgggggacgccatgttctggggcc
acgccacgtccgacgacctgaccaactgggaggaccagcccatcgccatcgccccgaagcgcaacgactccggcgccttctccggctccat
ggtggtggactacaacaacacctccggcttcttcaacgacaccatcgacccgcgccagcgctgcgtggccatctggacctacaacaccccgg
agtccgaggagcagtacatctcctacagcctggacggcggctacaccttcaccgagtaccagaagaaccccgtgctggccgccaactccac
ccagttccgcgacccgaaggtcttctggtacgagccctcccagaagtggatcatgaccgcggccaagtcccaggactacaagatcgagatct
actcctccgacgacctgaagtcctggaagctggagtccgcgttcgccaacgagggcttcctcggctaccagtacgagtgccccggcctgatcg
aggtccccaccgagcaggaccccagcaagtcctactgggtgatgttcatctccatcaaccccggcgccccggccggcggctcatcaaccagt
acttcgtcggcagcttcaacggcacccacttcgaggccttcgacaaccagtcccgcgtggtggacttcggcaaggactactacgccctgcaga
ccttcttcaacaccgacccgacctacgggagcgccctgggcatcgcgtgggcctccaactgggagtactccgccttcgtgcccaccaacccct
ggcgctcctccatgtccctcgtgcgcaagttctccctcaacaccgagtaccaggccaacccggagacggagctgatcaacctgaaggccgag
ccgatcctgaacatcagcaacgccggcccctggagccggttcgccaccaacaccacgttgacgaaggccaacagctacaacgtcgacctgt
ccaacagcaccggcaccctggagttcgagctggtgtacgccgtcaacaccacccagacgatctccaagtccgtgttcgcggacctctccctct
ggttcaagggcctggaggaccccgaggagtacctccgcatgggcttcgaggtgtccgcgtcctccttcttcctggaccgcgggaacagcaag
gtgaagttcgtgaaggagaacccctacttcaccaaccgcatgagcgtgaacaaccagccatcaagagcgagaacgacctgtcctactaca
aggtgtacggcttgctggaccagaacatcctggagctgtacttcaacgacggcgacgtcgtgtccaccaacacctacttcatgaccaccggga

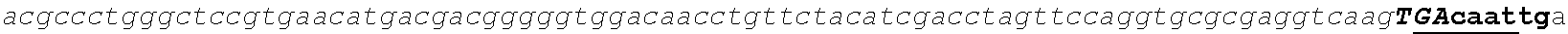

cgcccgcgcggcgcacctgacctgttctctcgagggcgcctgttctgccttgcgaaacaagcccctggagcatgcgtgcatgatcgtctctggcgc
cccgccgcgcggtttgtcgccctcgcgggcgccgcggccgcgggggcgcattgaaattgttgcaaaccccacctgacagattgagggcccagg
caggaaggcgttgagatggaggtacaggagtcaagtaactgaaagtttttatgataactaacaacaaagggtcgtttctggccagcgaatgacaag
aacaagattccacatttccgtgtagaggcttgccatcgaatgtgagcgggcgggccgcggacccgacaaaacccttacgacgtggtaagaaaaac
gtggcgggcactgtccctgtagcctgaagaccagcaggagacgatcggaagcatcacagcacaggatcccgcgtctcgaacagagcgcgcag
aggaacgctgaaggtctcgcctctgtcgcacctcagcgcggcatacaccacaataaccacctgacgaatgcgcaggacttcgtccattagcgaag
cgtccggttcacacacgtgccacgaggcgaggtggcaggtgacaatgatcggtggagctgatggtcgaaacgttcacagcctagggcagcagc
agctcggatagtatcgacacactctggacgctggtcgtgtgatggactgagccgccacacttgctgccttgacctgtgaatatccctgccgatttatc
aaacagcctcagtgtgatgatcagtgtgtacgcgcttagcgagttgctagctgcttgtgctatttgcgaataccacccccagcatccccttccctcgtt
tcatatcgcttgcatcccaaccgcaacttatctacgctgtcctgctatccctcagcgctgctcctgctcctgctcactgcccctcgcacagccaggtag
ggctccgcctgtattctcctggtactgcaacctgtaaaccagcactgcaatgctgatgcacgggaagtagtgggatgggaacacaaatggaaagct

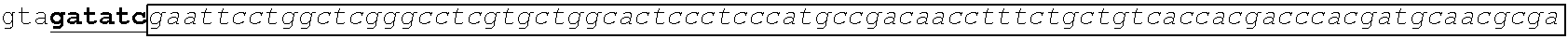

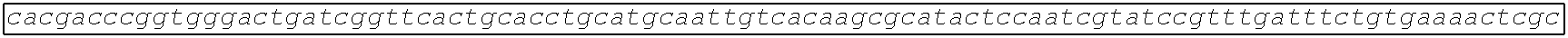

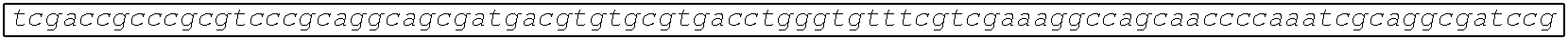

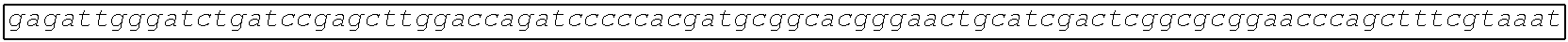

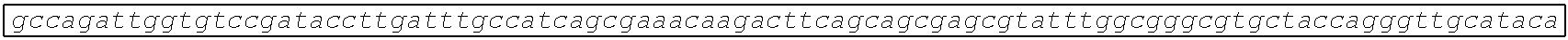

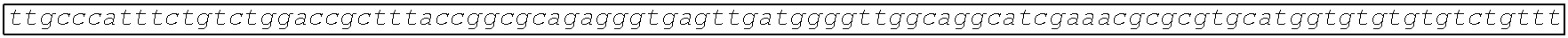

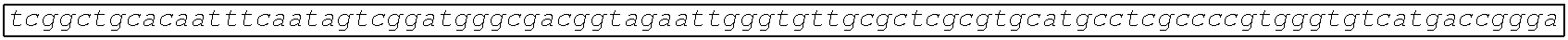

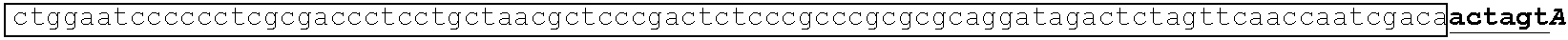

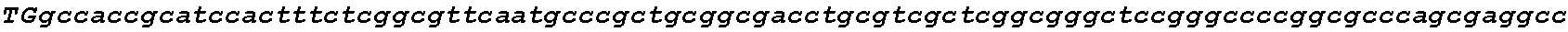

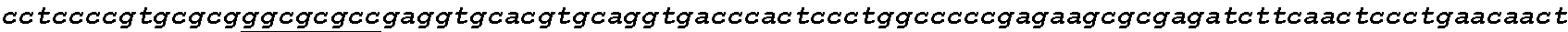

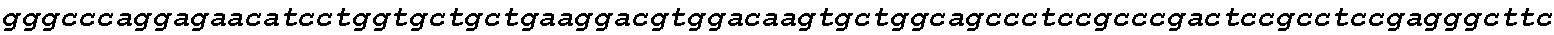

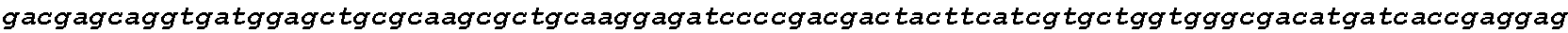

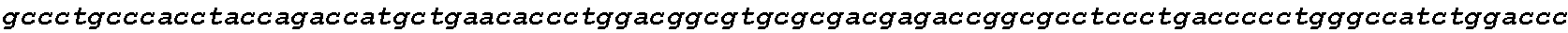

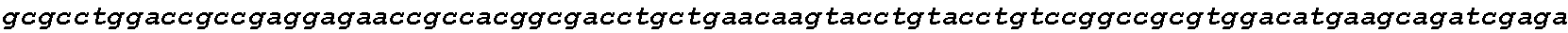

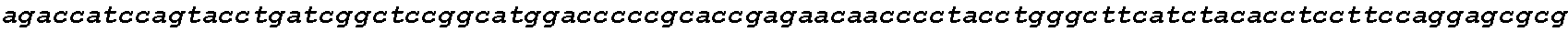

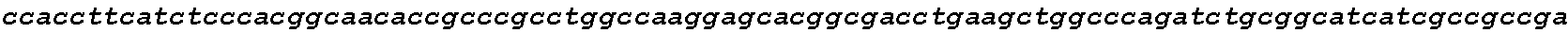

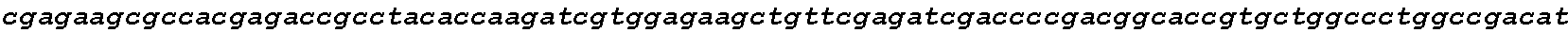

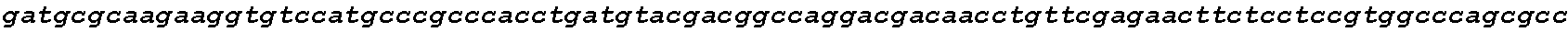

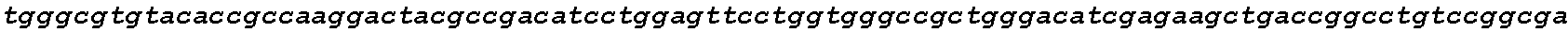

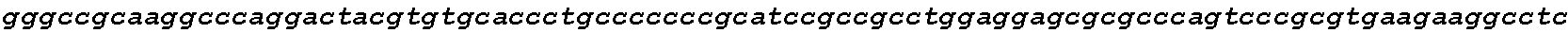

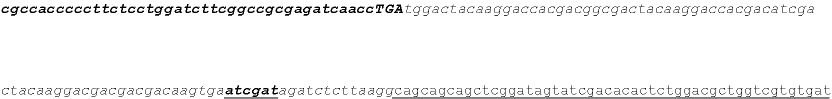

ggactgttgccgccacacttgctgccttgacctgtgaatatccctgccgcttttatcaaacagcctcagtgtgtttgatcttgtgtgtacgcgcttttgcga
gttgctagctgcttgtgctatttgcgaataccacccccagcatccccttccctcgtttcatatcgcttgcatcccaaccgcaacttatctacgctgtcctgc
tatccctcagcgctgctcctgctcctgctcactgcccctcgcacagccaggtagggctccgcctgtaactcctggtactgcaacctgtaaaccagca
ctgcaatgctgatgcacgggaagtagtgggatgggaacacaaatggaaagcttaattaagagctcttgttttccagaaggagttgctccttgagc
ctttcattctcagcctcgataacctccaaagccgctctaattgtggagggggttcgaatttaaaagcttggaatgttggttcgtgcgtctggaa
caagcccagacttgttgctcactgggaaaaggaccatcagctccaaaaaacttgccgctcaaaccgcgtacctctgctttcgcgcaatctg
ccctgttgaaatcgccaccacattcatattgtgacgcttgagcagtctgtaattgcctcagaatgtggaatcatctgccccctgtgcgagccc
atgccaggcatgtcgcgggcgaggacacccgccactcgtacagcagaccattatgctacctcacaatagttcataacagtgaccatatttc
tcgaagctccccaacgagcacctccatgctctgagtggccaccccccggccctggtgcttgcggagggcaggtcaaccggcatggggcta
ccgaaatccccgaccggatcccaccacccccgcgatgggaagaatctctccccgggatgtgggcccaccaccagcacaacctgctggcc
caggcgagcgtcaaaccataccacacaaatatccttggcatcggccctgaattccttctgccgctctgctacccggtgcttctgtccgaagc
aggggttgctagggatcgctccgagtccgcaaacccttgtcgcgtggcggggcttgttcgagcttgaagagc
SEQ ID NO: 142
catatgcggtgtgaaataccgcacagatgcgtaaggagaaaataccgcatcaggcgccattcgccattcaggctgcgcaactgttgg
gaagggcgatcggtgcgggcctcttcgctattacgccagctggcgaaagggggatgtgctgcaaggcgattaagttgggtaacgcc
agggattcccagtcacgacgagtaaaacgacggccagtgaattgatgcatgctatcgcgaaggtcattttccagaacaacgacca
tggcttgtcttagcgatcgctcgaatgactgctagtgagtcgtacgctcgacccagtcgctcgcaggagaacgcggcaactgcc
gagcttcggcttgccagtcgtgactcgtatgtgatcaggaatcattggcattggtagcattataattcggcttccgcgctgtttat
gggcatggcaatgtctcatgcagtcgaccttagtcaaccaattctgggtggccagctccgggcgaccgggctccgtgtcgccg
ggcaccacctcctgccatgagtaacagggccgccactcctcccgacgttggcccactgaataccgtgtcttggggccctacat
gatgggctgcctagtcgggcgggacgcgcaactgcccgcgcaatctgggacgtggtctgaatcctccaggcgggtttccccga
gaaagaaagggtgccgatttcaaagcagagccatgtgccgggccagtggcctgtgttggcgcctatgtagtcaccccccctc
acccaattgtcgccagtttgcgcaatccataaactcaaaactgcagcttctgagagcgctgttcaagaacacctaggggtttg

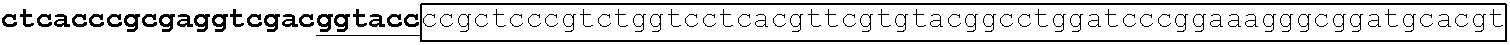

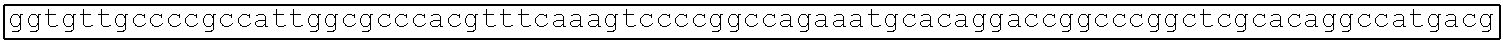

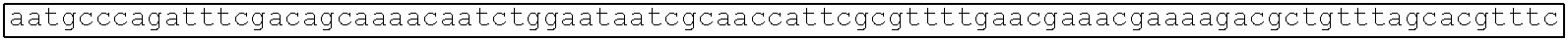

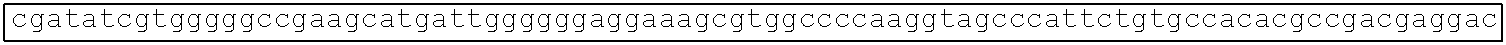

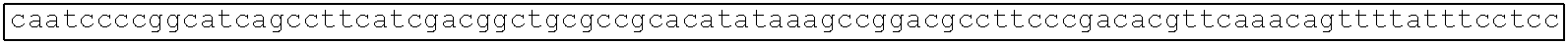

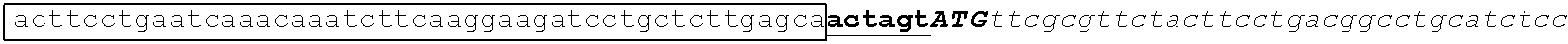

ctgaagggcgtgttcggcgtaccccctcctacaacggcctgggcctgacgccccagatgggctgggacaactggaacacgttcg
cctgcgacgtaccgagcagagagaggacacggccgaccgcataccgacctgggcctgaaggacatgggctacaagtaca
tcatcctggacgactgctggtcctccggccgcgactccgacggcttcctggtcgccgacgagcagaagttccccaacggcatggg
ccacgtcgccgaccacctgcacaacaactccttcctgttcggcatgtactcctccgcgggcgagtacacgtgcgccggctaccccg
gctccctgggccgcgaggaggaggacgcccagttcttcgcgaacaaccgcgtggactacctgaagtacgacaactgctacaac
aagggccagttcggcacgcccgagatctcctaccaccgctacaaggccatgtccgacgccagaacaagacgggccgccccat
cttctactccctgtgcaactggggccaggacctgaccttctactggggaccggcatcgcgaactcctggcgcatgtccggcgacgt
cacggcggagttcacgcgccccgactcccgctgcccctgcgacggcgacgagtacgactgcaagtacgccggatccactgac
catcatgaacatcctgaacaaggccgcccccatgggccagaacgcgggcgtcggcggctggaacgacctggacaacctggag
gtcggcgtcggcaacctgacggacgacgaggagaaggcgcacttctccatgtgggccatggtgaagtcccccctgatcatcggc
gcgaacgtgaacaacctgaaggcctcctcctactccatctactcccaggcgtccgtcatcgccatcaaccaggactccaacggca
tccccgccacgcgcgtaggcgctactacgtgtccgacacggacgagtacggccagggcgagatccagatgtggtccggccccc
tggacaacggcgaccaggtcgtggcgctgctgaacggcggctccgtgtcccgccccatgaacacgaccaggaggagatatctt
cgactccaacctgggaccaagaagagacctccacctgggacatctacgacctgtgggcgaaccgcgtcgacaactccacggc
gtccgccatcctgggccgcaacaagaccgccaccggcatcctgtacaacgccaccgagcagtcctacaaggacggcctgtcca
agaacgacacccgcctgttcggccagaagatcggctccctgtcccccaacgcgatcctgaacacgaccgtccccgcccacggc

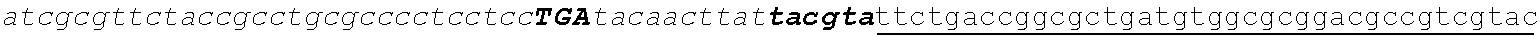

tctttcagactttactcttgaggaattgaacctttctcgcttgctggcatgtaaacattggcgcaattaattgtgtgatgaagaaagggtggc
acaagatggatcgcgaatgtacgagatcgacaacgatggtgattgttatgaggggccaaacctggctcaatcttgtcgcatgtccggc
gcaatgtgatccagcggcgtgactctcgcaacctggtagtgtgtgcgcaccgggtcgctttgattaaaactgatcgcattgccatcccgt
caactcacaagcctactctagctcccattgcgcactcgggcgcccggctcgatcaatgttctgagcggagggcgaagcgtcaggaaa

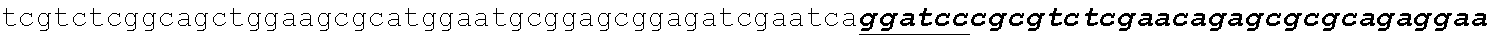

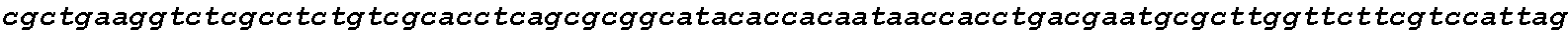

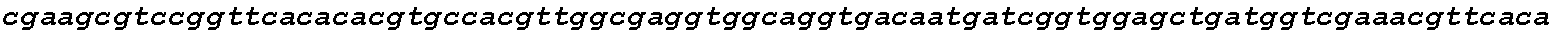

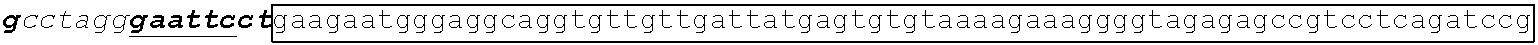

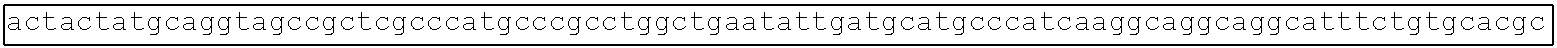

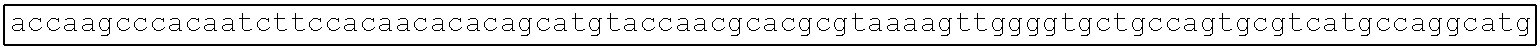

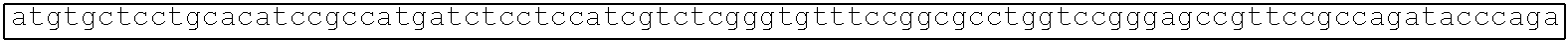

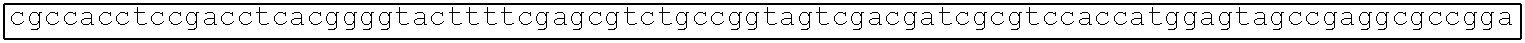

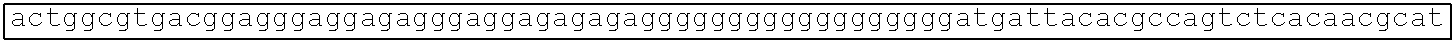

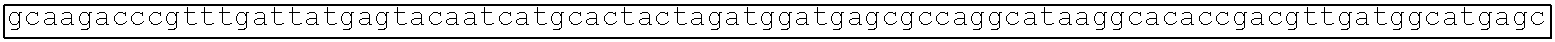

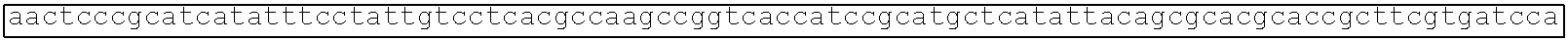

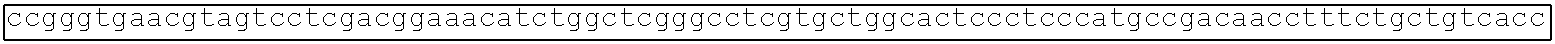

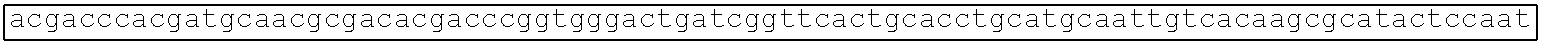

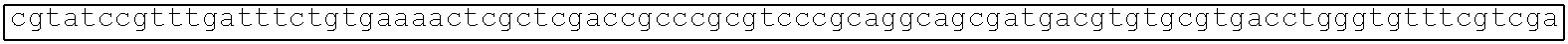

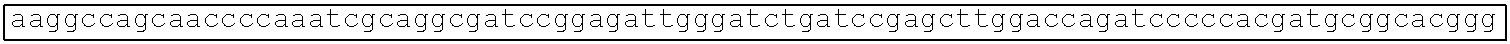

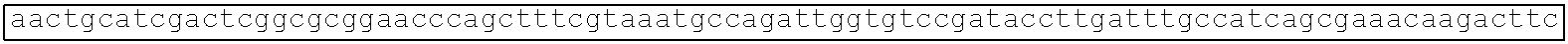

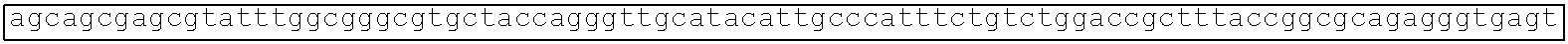

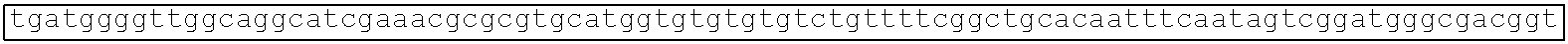

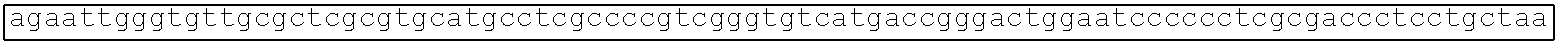

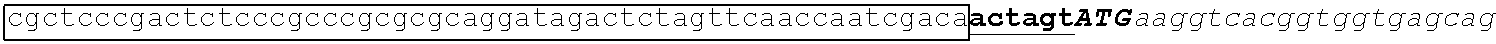

gtccggcagggaggtgacaaggcccccaggacctgccggactccgccacggtcgctgacctccaggaggccttccacaagc
gcgcgaagaagttttatcccagccgccagcggctgacccttccggtggcccccggaccaaggacaagccggtggtgctgaact
cgaagaagagcctcaaggagtactgcgacggtaacaccgactcgacacggtggtgtttaaggacttgggcgcgcaggtacct
accgcaccagttcttatcgagtacctgggccccctgctgatctaccccgtatctactacttccagtctataagtacctgggctacgg
cgaggaccgcgtcatccacccggtgcagacgtatgccatgtactactggtgatccactactttaagcgcattatggagacgttcttc
gtgcaccgatcagccacgccacctcgcccatcggtaacgtatccgcaactmcctactactggacgttcggcgcctacatcgct
tactacgtgaaccaccccctgtacacccccgtgagcgacttgcagatgaagatcggcttcgggttcggcctcgtgtttcaggtggcg
aacttctactgccacatcctgctgaagaatctgcgcgacccgaacggcagcggcggttaccagatcccgcgcggcttcctgttcaa
catcgtcacgtgcgcgaactacaccacggagatctaccagtggctcggattaacatcgccacgcagaccatcgccggctacgtg
ttcctcgcggtggccgccagattatgaccaactgggccacggcaagcactcgcggaccggaagatatcgacggcaaggacg

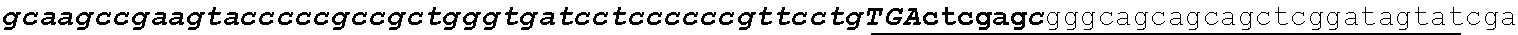

cacactctggacgctggtcgtgtgatggactgttgccgccacacagctgccagacctgtgaatatccctgccgctatatcaaacagcc
tcagtgtgtagatcagtgtgtacgcgcattgcgagagctagctgcagtgctatttgcgaataccacccccagcatccccttccctcgat
catatcgcttgcatcccaaccgcaacttatctacgctgtcctgctatccctcagcgctgctcctgctcctgctcactgcccctcgcacagc
cttggtttgggctccgcctgtaactcctggtactgcaacctgtaaaccagcactgcaatgctgatgcacgggaagtagtgggatggga
acacaaatggaaagctgtagagctcctcactcagcgcgcctgcgcggggatgcggaacgccgccgccgccttgtcttttgcacgc
gcgactccgtcgcttcgcgggtggcacccccattgaaaaaaacctcaattctgtttgtggaagacacggtgtacccccaaccac
ccacctgcacctctattattggtattattgacgcgggagcgggcgttgtactctacaacgtagcgtctctggttttcagctggctc
ccaccattgtaaattcttgctaaaatagtgcgtggttatgtgagaggtatggtgtaacagggcgtcagtcatgttggttttcgtgc
tgatctcgggcacaaggcgtcgtcgacgtgacgtgcccgtgatgagagcaataccgcgctcaaagccgacgcatggcctttac
tccgcactccaaacgactgtcgctcgtatttttcggatatctattttttaagagcgagcacagcgccgggcatgggcctgaaagg
cctcgcggccgtgctcgtggtgggggccgcgagcgcgtggggcatcgcggcagtgcaccaggcgcagacggaggaacgcat
ggtgagtgcgcatcacaagatgcatgtcttgttgtctgtactataatgctagagcatcaccaggggcttagtcatcgcacctgct
ttggtcattacagaaattgcacaagggcgtcctccgggatgaggagatgtaccagctcaagctggagcggcttcgagccaag
caggagcgcggcgcatgacgacctacccacatgcgaagagcctctaga
SEQ ID NO: 143

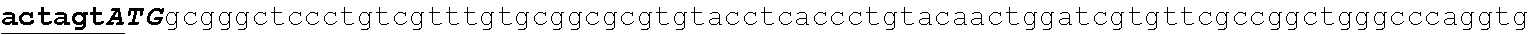

ctgtactagccgtcaagacgctcaaggagtccggccacgagaacgtgtacgacgccgtggagaagcccctccagctggcgcaaac
cgccgcggtcctggagatcctccacggcctggtcggcctcgtcaggagcccggtctcggccaccctgccgcagatcgggagccgc
ctctactgacctggggcattctgtattccacccggaggtccagagccactactggtgacctccctcgtgatcagctggtcgatcacgg
aaatcatccgctacagatcacggcctgaaggaggcgctgggcacgcgcccagctggcacctgtggctccgctattcgagctactg
gtgctctaccccaccggcatcacctccgaggtcggcctcatctacctggccctgccgcacatcaagacgtcggagatgtactccgtcc
gcatgcccaacaccagaacttaccacgacatactacgccacgattctcgtcctcgcgatctacgtccccggacgccccacatgtacc

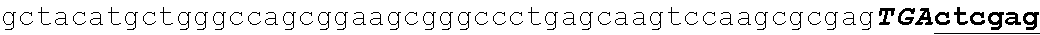

SEQ ID NO: 144

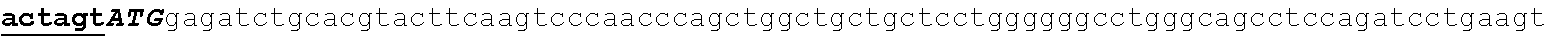

cgacgttctccctcctgaagagcctgtacatctacttcctgcgccccggcaagaacctccgccgctacgggtcctgggccattatcacc
ggcccgaccgacggcatcggcaaggccatgcgaccagctggcccacaagggcctgaacctggtgctggtggcgcgcaacccgg
acaagctgaaggacgtctccgacagcatcaggtccaagcatagcaacgtgcagatcaagacggtgatcatggacatagcggcgac
gttgacgacggcgtccgccgcatcaaggagaccatcgaggggctggaggtgggcatcctgatcaacaatgccggcatgtcctaccc
gtacgcgaagtactacacgaggtcgacgaggagctcgtcaacggcctcatcaaaatcaacgtcgagggcacgaccaaggtgaccc
aggccgtgctgccgggcatgctggagcgcaagcgcggcgccatcgtcaacatgggcagcggcgcggccgccctgatcccgtcgt
accccactacagcgtgtatgccggcgcgaagacgtacgtggaccagacacccggtgcctgcacgtcgagtacaagaagagcggc
attgacgtccagtgccaggtcccgctctacgtggccacgaagatgacgaagatccgccgcgcctccacctggtcgcctcccccgag
ggctacgccaaggccgccctgcggttcgtggggtacgaggcccggtgcaccccctactggccgcacgccctgatgggctacgtcgt
ctccgccctgccccagtccgtgacgagtccacaacatcaagcgctgcctgcagatccgcaagaagggcatgctgaaggattcgcgg

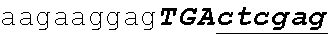

SEQ ID NO: 145
gatttctatcatcaagtttctcatatgtttcacgcgttgctcacaacaccggcaaatgcgttgttgttccctgtttttacaccttgcc
agagcctggtcaaagcttgacagtttgaccaaattcaggtggcctcatctctctcgcactgatagacattgcagatttggaaga
cccagtcagtacactacatgcacagccgtttgctcctgcgccatgaacttgccacttttgtgcgccggtcgggggtgatagctcg
gcagccgccgatcccaaaggtcccgcggcccaggggcacgagaacccccgacacgattaaatagccaaaatcagttagaac
ggcacctccaccctacccgaatctgacagggtcatcaagcgcgcgaaacaacggcgagggtgcgttcgggaagcgcgcgta
gttgacgcaagaagcctgggtcaggctgggagggccgcgagaagatcgcttcctgccgagtctgcacccacgcctcgagcgc
accgtccgcgaacaaccaacccctttgcgcgagccctgacattctttcaattgccaaggatgcacatgtgacacgtatagccat
tcggctttgtttgtgcctgcttgactcgcgtcatttaattgatttgtgccggtgagccgggagtcggccactcgtctccgagccgc
agtcccggcgccagtcccccggcctctgatctgggtccggaagggttggtataggagcggtctcggctatctgaagcccattac

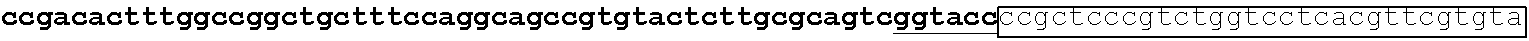

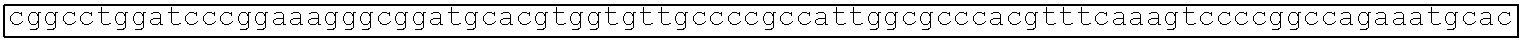

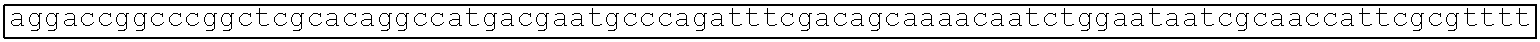

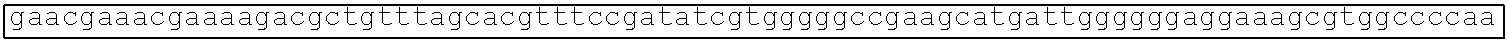

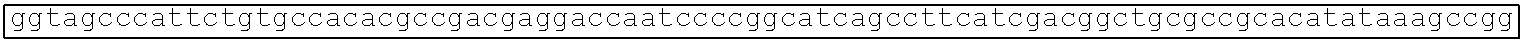

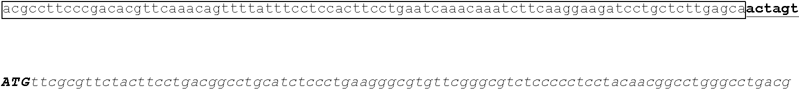

ATGttcgcgttctacttcctgacggcctgcatctccctgaagggcgtgttcggcgtaccccctcctacaacggcctgggcctgacg
ccccagatgggctgggacaactggaacacgttcgcctgcgacgtctccgagcagctgctgctggacacggccgaccgcatctcc
gacctgggcctgaaggacatgggctacaagtacatcatcctggacgactgctggtcctccggccgcgactccgacggcttcctgg
tcgccgacgagcagaagttccccaacggcatgggccacgtcgccgaccacctgcacaacaactccttcctgttcggcatgtactc
ctccgcgggcgagtacacgtgcgccggctaccccggctccctgggccgcgaggaggaggacgcccagttcttcgcgaacaacc
gcgtggactacctgaagtacgacaactgctacaacaagggccagttcggcacgcccgagatctcctaccaccgctacaaggcc
atgtccgacgccctgaacaagacgggccgccccatatctactccctgtgcaactggggccaggacctgaccttctactggggctc
cggcatcgcgaactcctggcgcatgtccggcgacgtcacggcggagttcacgcgccccgactcccgctgcccctgcgacggcga
cgagtacgactgcaagtacgccggcttccactgctccatcatgaacatcctgaacaaggccgcccccatgggccagaacgcgg
gcgtcggcggctggaacgacctggacaacctggaggtcggcgtcggcaacctgacggacgacgaggagaaggcgcacttctc
catgtgggccatggtgaagtcccccctgatcatcggcgcgaacgtgaacaacctgaaggcctcctcctactccatctactcccagg
cgtccgtcatcgccatcaaccaggactccaacggcatccccgccacgcgcgtctggcgctactacgtgtccgacacggacgagt
acggccagggcgagatccagatgtggtccggccccctggacaacggcgaccaggtcgtggcgctgctgaacggcggctccgtg
tcccgccccatgaacacgaccctggaggagatatcttcgactccaacctgggctccaagaagctgacctccacctgggacatct
acgacctgtgggcgaaccgcgtcgacaactccacggcgtccgccatcctgggccgcaacaagaccgccaccggcatcctgtac
aacgccaccgagcagtcctacaaggacggcctgtccaagaacgacacccgcctgacggccagaagatcggctccctgtcccc

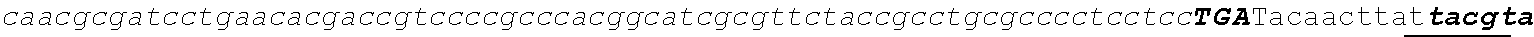

ttctgaccggcgctgatgtggcgcggacgccgtcgtactcatcagacatactcagaggaattgaaccatctcgcttgctggcatgta
aacattggcgcaattaattgtgtgatgaagaaagggtggcacaagatggatcgcgaatgtacgagatcgacaacgatggtgattgttat
gaggggccaaacctggctcaatcttgtcgcatgtccggcgcaatgtgatccagcggcgtgactctcgcaacctggtagtgtgtgcgca
ccgggtcgctttgattaaaactgatcgcattgccatcccgtcaactcacaagcctactctagctcccattgcgcactcgggcgcccggct
cgatcaatgttctgagcggagggcgaagcgtcaggaaatcgtctcggcagctggaagcgcatggaatgcggagcggagatcgaat

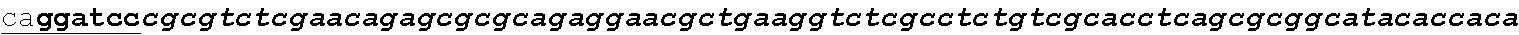

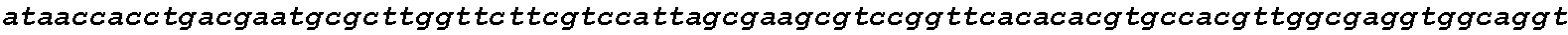

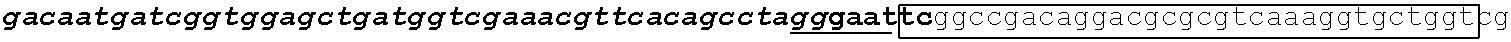

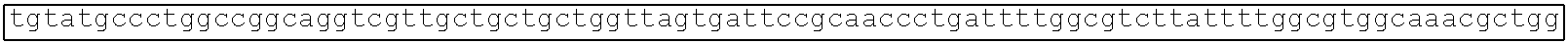

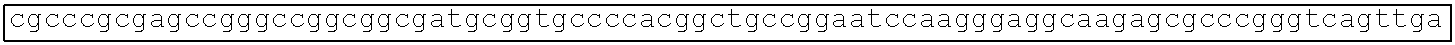

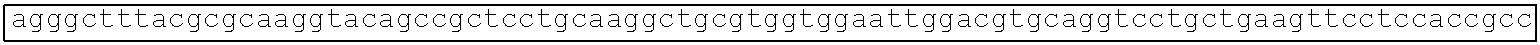

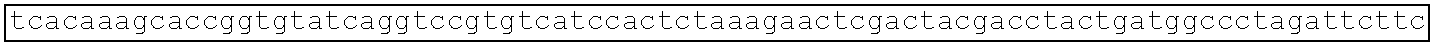

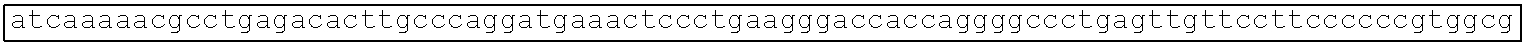

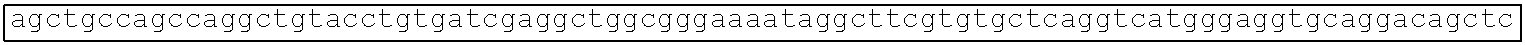

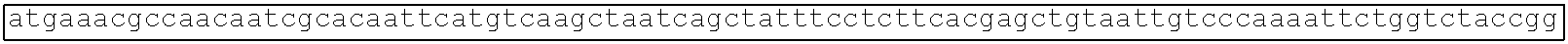

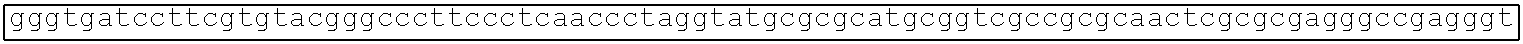

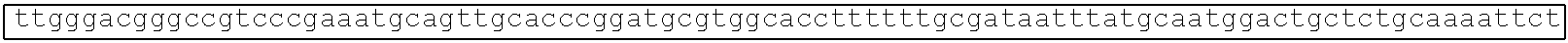

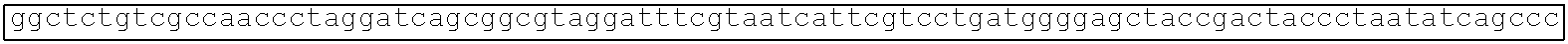

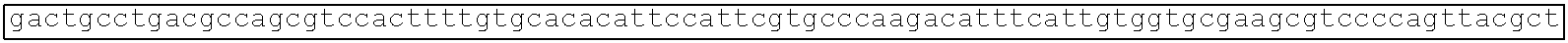

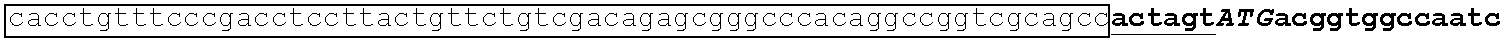

ccccggaagccccgttcgacagcgagggttcctcgctggcgcccgacaatgggtccagcaagcccaccaagctgagctccac
ccggtccttgctgtccatctcctaccgggagctctcgcgttccaagtgcgtgcaggggcgggggcaccttttgttggtgttgtttg
ggcgggcctcagcactggggtggaggaagaatgcgtgagtgtgcttgcacacctcggcggtttaagatgtaatgcgccaattt
cttgctgatgcattcctagacacaaagagtactcattcgagtctcatcgcggttgtgcgctcctcactccgtgcagccagcagtc
gcggtcgttcacttcgcggggggtgccagggaggacggacgtttcggatgagaggagcgccgcatcctcgagtggcagggc
gatcgcgccatccacaggtcggttgggtgggaaagggggggcgttggggtcaggtcagaagtcgtgaagttacaggcctgca
tttgcacatcctgcgcgcgcctctggccgcttgtcttaagacccttgcactcgcttcctcatgaacccccatgaactccctcctgc
accccacagcgtgctggtggccaacaacggtctggcggcggtcaagttcatccggtcgatccggtcgtggtcgtacaagacgt
ttgggaacgagcgtgcggtgaagctgatcgcgatggcgacgcccgaggacatgcgcggacgcggagcacatccgcatgg
cggaccagtttgtggaggtccccggcggcaagaacgtgcagaactacgccaacgtgggcctgatcacctcggtggcggtgcg
caccggggtggacgcggtgcctgcagg
SEQ ID NO: 146
Gattcatatcatcaaatttcgcatatgtttcacgagttgctcacaacatcggcaaatgcgttgttgttccctgtttttacaccttgccagggcc
tggtcaaagcttgacagtttgaccaaattcaggtggcctc atctctttcgcactgatagacattgcagatttggaagacccagccagtaca
ttacatgcacagccatttgctcctgcaccatgaacttgccacttttgtgcgccggtcgggggtgatagctcggcagccgccgatcccaa
aggtcccgcggcccaggggcacgagaccccccgacacgattaaatagccaaaatcagtcagaacggcacctccaccctacccgaa
tctgacaaggtcatcaaacgcgcgaaacaacggcgagggtgcgttcgggaagcgcgcgtagttgacgcaagaagcctgggtcagg
ctggagggccgcgagaagatcgcttcctgccgagtctgcacccacgcctcgagcgcaccgtccgcgaacaaccaaccccttttcgc
gagccctggcattctttcaattgccaaggatgcacatgtgacacgtatagccattcggctttgtttgtgcctgcttgactcgcgccatttaat
tgttttgtgccggtgagccgggagtcggccactcgtctccgagccgcagtcccggcgccagtcccccggcctctgatctgggtccgg
aagggttggtataggagcagtctcggctatctgaagcccgttaccagacactttggccggctgctttccaggcagccgtgtactcttgc
gcagtcggtacc
SEQ ID NO: 147
actagtATGacggtggccaatcccccggaagccccgttcgacagcgagggttcctcgctggcgcccgacaatgggtccagcaag
cccaccaagctgagctccacccggtccctgctgtccatctcctaccgggagctctcgcgttccaagtgcgtacaggggcgagggcac
cttttgttggtgttgtttgggcgggcctcggtactgggaggaggaggaatgcgtgcacacctctgcggttttagatgcaatgcgacaagt
gcctgctgatgcattttctagacatgaagcatctcgtattcgagtctcaacgcgggtgtgcgctcctcactccgtgcagccagcagtcgc
ggtcgttcacttcgcggggggtgccagggaggacggacgtttcggatgagctggagcgccgcatcctcgagtggcagggcgatcg
cgccatccacaggtcggttgggtgggaaagggggagtaccggggtcaggtcagaagtcgtgcatttacaggcatgcatctgcacatc
gtgcgcacgcgcacgtctttggccgcttgtctcaagactcttgcactcgtttcctcatgcaccataatcaattccctcccccctcgcaaact
cacagcgtgctggtggccaacaacggtctggcggcggtcaagttcatccggtcgatccggtcgtggtcgtacaagacgtttgggaac
gagcgcgcggtgaagctgattgcgatggcgacgcccgagggcatgcgcgcggacgcggagcacatccgcatggcggaccagttt
gtggaggtccccggcggcaagaacgtgcagaactacgccaacgtgggcctgatcacctcggtggcggtgcgcaccggggtggac
gcggtgcctgcagg
SEQ ID NO: 148

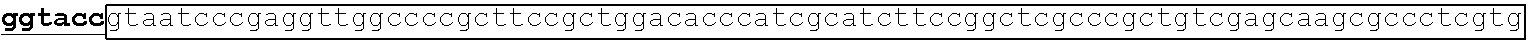

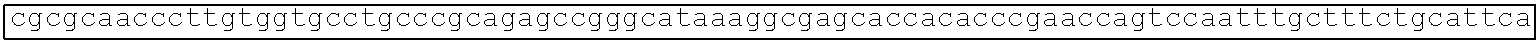

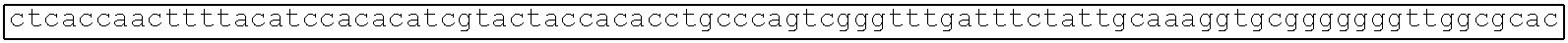

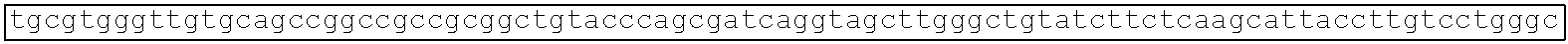

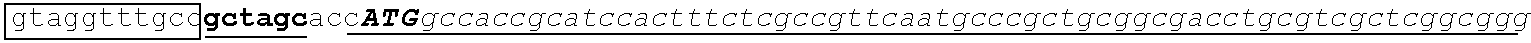

ctccgggccccggcgcccagcgaggcccctccccgtgcgcgggcgcgccgtccaggccgcggccacccgcttcaagaaggag
acgacgaccacccgcgccacgctgacgttcgacccccccacgaccaactccgagcgcgccaagcagcgcaagcacaccatc
gacccctcctcccccgacttccagcccatcccctccttcgaggagtgcttccccaagtccacgaaggagcacaaggaggtggtgc
acgaggagtccggccacgtcctgaaggtgcccttccgccgcgtgcacctgtccggcggcgagcccgccttcgacaactacgaca
cgtccggcccccagaacgtcaacgcccacatcggcctggcgaagctgcgcaaggagtggatcgaccgccgcgagaagctggg
cacgcccgctacacgcagatgtactacgcgaagcagggcatcatcacggaggagatgctgtactgcgcgacgcgcgagaag
ctggaccccgagttcgtccgctccgaggtcgcgcggggccgcgccatcatcccctccaacaagaagcacctggagctggagcc
catgatcgtgggccgcaagttcctggtgaaggtgaacgcgaacatcggcaactccgcgtggcctcctccatcgaggaggaggt
ctacaaggtgcagtgggccaccatgtggggcgccgacaccatcatggacctgtcacgggccgccacatccacgagacgcgcg
agtggatcctgcgcaactccgcggtccccgtgggcaccgtcccatctacaggcgctggagaaggtggacggcatcgcggag
aacctgaactgggaggtgttccgcgagacgctgatcgagcaggccgagcagggcgtggactacttcacgatccacgcgggcgt
gctgctgcgctacatccccctgaccgccaagcgcatgacgggcatcgtgcccgcggcggctccatccacgcgaagtggtgcctg
gcctaccacaggagaacttcgcctacgagcactgggacgacatcctggacatctgcaaccagtacgacgtcgccctgtccatc
ggcgacggcctgcgccccggctccatctacgacgccaacgacacggcccagttcgccgagctgctgacccagggcgagctgac
gcgccgcgcgtgggagaaggacgtgcaggtgatgaacgagggccccggccacgtgcccatgcacaagatccccgagaacat
gcagaagcagctggagtggtgcaacgaggcgcccttctacaccctgggccccctgacgaccgacatcgcgcccggctacgacc
acatcacctccgccatcggcgcggccaacatcggcgccctgggcaccgccctgctgtgctacgtgacgcccaaggagcacctgg
gcctgcccaaccgcgacgacgtgaaggcgggcgtcatcgcctacaagatcgccgcccacgcggccgacctggccaagcagca
cccccacgcccaggcgtgggacgacgcgctgtccaaggcgcgcttcgagttccgctggatggaccagttcgcgctgtccctggac
cccatgacggcgatgtccttccacgacgagacgctgcccgcggacggcgcgaaggtcgcccattctgctccatgtgcggcccc
aagttctgctccatgaagatcacggaggacatccgcaagtacgccgaggagaacggctacggctccgccgaggaggccatcc
gccagggcatggacgccatgtccgaggagttcaacatcgccaagaagacgatctccggcgagcagcacggcgaggtcggcg

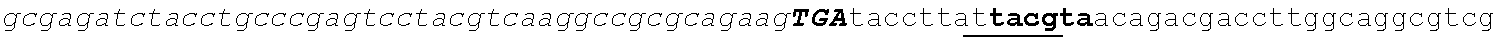

ggtaggaggtggtggtgatggcgtctcgatgccatcgcacgcatccaacgaccgtatacgcatcgtccaatgaccgtcggtgtcctc
tctgcctccgttttgtgagatgtctcaggcttggtgcatcctcgggtggccagccacgttgcgcgtcgtgctgcttgcctctcttgcgcctc
tgtggtactggaaaatatcatcgaggcccgtttttttgctcccatttcctttccgcacatcttgaaagcaaacgacaaacgaagcagcaa
gcaaagagcacgaggacggtgaacaagtctgtcacctgtatacatctatttccccgcgggtgcacctactctctctcctgccccggcag
agtcagctgccttacgtgacggatcc
SEQ ID NO: 149
catatgtttcacgcgttgctcacaacaccggcaaatgcgttgttgttccctgtttttacaccttgccagagcctggtcaaagcttg
acagtttgaccaaattcaggtggcctcatctctctcgcactgatagacattgcagatttggaagacccagtcagtacactacatg
cacagccgtttgctcctgcgccatgaacttgccacttttgtgcgccggtcgggggtgatagctcggcagccgccgatcccaaag
gtcccgcggcccaggggcacgagaacccccgacacgattaaatagccaaaatcagttagaacggcacctccaccctacccg
aatctgacagggtcatcaagcgcgcgaaacaacggcgagggtgcgttcgggaagcgcgcgtagttgacgcaagaagcctgg
gtcaggctgggagggccgcgagaagatcgcttcctgccgagtctgcacccacgcctcgagcgcaccgtccgcgaacaacca
acccctttgcgcgagccctgacattctttcaattgccaaggatgcacatgtgacacgtatagccattcggctttgtttgtgcctgct
tgactcgcgtcatttaattgatttgtgccggtgagccgggagtcggccactcgtctccgagccgcagtcccggcgccagtcccc
cggcctctgatctgggtccggaagggttggtataggagcggtctcggctatctgaagcccattacccgacactttggccggctg

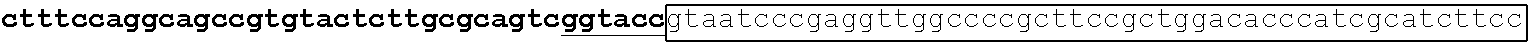

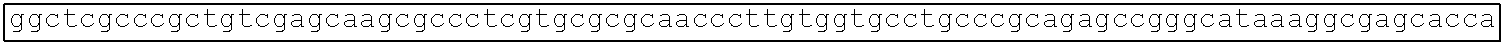

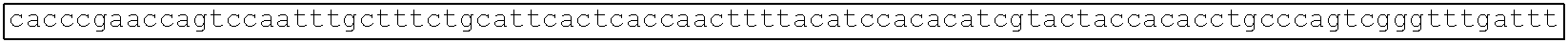

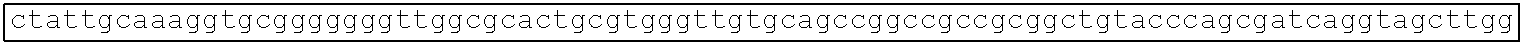

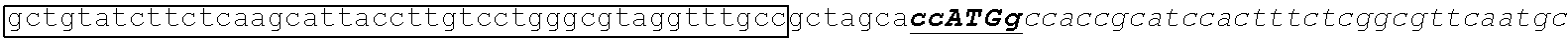

ccgctgcggcgacctgcgtcgctcggcgggctccgggccccggcgcccagcgaggcccctcccccgtgcgcgggcgcgccgtcc
aggccgcggccacccgcttcaagaaggagacgacgaccacccgcgccacgctgacgttcgacccccccacgaccaactccga
gcgcgccaagcagcgcaagcacaccatcgacccctcctcccccgacttccagcccatcccctccttcgaggagtgcttccccaag
tccacgaaggagcacaaggaggtggtgcacgaggagtccggccacgtcctgaaggtgcccttccgccgcgtgcacctgtccgg
cggcgagcccgccttcgacaactacgacacgtccggcccccagaacgtcaacgcccacatcggcctggcgaagctgcgcaag
gagtggatcgaccgccgcgagaagctgggcacgccccgctacacgcagatgtactacgcgaacagggcatcatcacggagg
agatgctgtactgcgcacgcgcgagaagctggaaaagagttcgtccgctccgaggtcgcgcggggccgcgccatcatcccct
ccaacaagaagcacctggagctggagcccatgatcgtgggccgcaagttcctggtgaaggtgaacgcgaacatcggcaactcc
gccgtggcctcctccatcgaggaggaggtctacaaggtgcagtgggccaccatgtggggcgccgacaccatcatggacctgtcc
acgggccgccacatccacgagacgcgcgagtggatcctgcgcaactccgcggtccccgtgggcaccgtccccatctaccaggc
gctggagaaggtggacggcatcgcggagaacctgaactgggaggtgttccgcgagacgctgatcgagcaggccgagcaggg
cgtggactacttcacgatccacgcgggcgtgctgctgcgctacatccccctgaccgccaagcgcatgacgggcatcgtgtcccgc
ggcggctccatccacgcgaagtggtgcctggcctaccacaaggagaacttcgcctacgagcactgggacgacatcctggacatc
tgcaaccagtacgacgtcgccctgtccatcggcgacggcctgcgccccggctccatctacgacgccaacgacacggcccagttc
gccgagctgctgacccagggcgagctgacgcgccgcgcgtgggagaaggacgtgcaggtgatgaacgagggccccggccac
gtgcccatgcacaagatccccgagaacatgcagaagcagctggagtggtgcaacgaggcgcccttctacaccctgggccccct
gacgaccgacatcgcgcccggctacgaccacatcacctccgccatcggcgcggccaacatcggcgccctgggcaccgccctgc
tgtgctacgtgacgcccaaggagcacctgggcctgcccaaccgcgacgacgtgaaggcgggcgtcatcgcctacaagatcgcc
gcccacgcggccgacctggccaagcagcacccccacgcccaggcgtgggacgacgcgctgtccaaggcgcgcttcgagttcc
gctggatggaccagttcgcgctgtccctggaccccatgacggcgatgtccttccacgacgagacgctgcccgcggacggcgcga
aggtcgcccacttctgctccatgtgcggccccaagttctgctccatgaagatcacggaggacatccgcaagtacgccgaggaga
acggctacggctccgccgaggaggccatccgccagggcatggacgccatgtccgaggagttcaacatcgccaagaagacgat

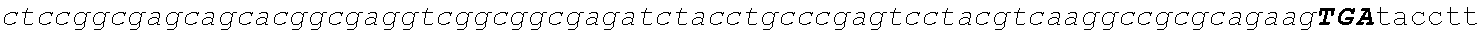

attacgtaacagacgaccttggcaggcgtcgggtagggaggtggtggtgatggcgtctcgatgccatcgcacgcatccaacgaccg
tatacgcatcgtccaatgaccgtcggtgtcctctctgcctccgttttgtgagatgtctcaggcttggtgcatcctcgggtggccagccacg
ttgcgcgtcgtgctgcttgcctctcttgcgcctctgtggtactggaaaatatcatcgaggcccgtttttttgctcccatttcctttccgctacat
cttgaaagcaaacgacaaacgaagcagcaagcaaagagcacgaggacggtgaacaagtctgtcacctgtatacatctatttccccgc
gggtgcacctactctctctcctgccccggcagagtcagctgccttacgtgacggatcccgcgtctcgaacagagcgcgcagagga
acgctgaaggtctcgcctctgtcgcacctcagcgcggcatacaccacaataaccacctgacgaatgcgcttggttcttcgtcca
ttagcgaagcgtccggttcacacacgtgccacgttggcgaggtggcaggtgacaatgatcggtggagctgatggtcgaaacg

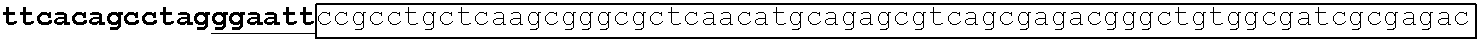

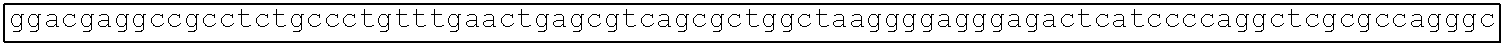

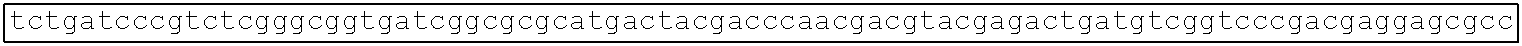

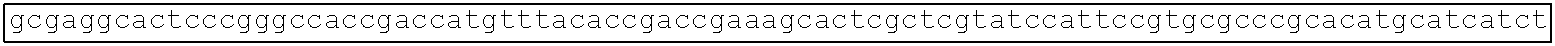

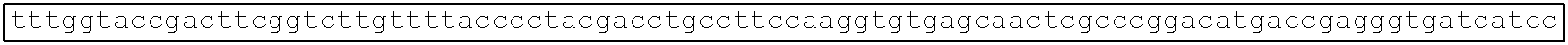

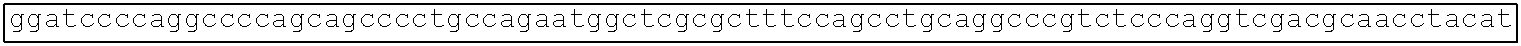

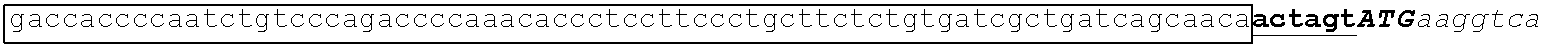

cggtggtgagcaggtccggcagggaggtgctcaaggcccccctggacctgccggactccgccacggtcgctgacctccaggag
gccttccacaagcgcgcgaagaagttttatcccagccgccagcggctgaccctgccggtggcccccggctccaaggacaagcc
ggtggtgctgaactcgaagaagagcctcaaggagtactgcgacggtaacaccgactcgctcacggtggtgtttaaggacttggg
cgcgcaggtctcctaccgcaccctgttcttcttcgagtacctgggccccctgctgatctaccccgtcttctactacttccctgtctataag
tacctgggctacggcgaggaccgegtcatccacccggtgcagacgtatgccatgtactactggtgcttccactactttaagcgcatt
atggagacgttcttcgtgcaccgcttcagccacgccacctcgcccatcggtaacgtcttccgcaactgcgcctactactggacgttc
ggcgcctacatcgcttactacgtgaaccaccccctgtacacccccgtgagcgacttgcagatgaagatcggcttcgggttcggcct
cgtgtttcaggtggcgaacttctactgccacatcctgctgaagaatctgcgcgacccgaacggcagcggcggttaccagatccg
cgcggcttcctgttcaacatcgtcacgtgcgcgaactacaccacggagatctaccagtggctcggctttaacatcgccacgcagac
catcgccggctacgtgttcctcgcggtggccgccctgattatgaccaactgggccctcggcaagcactcgcggctccggaagatct

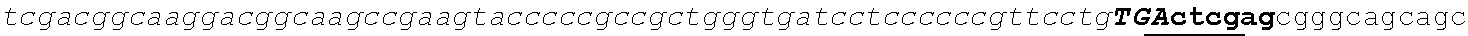

agctcggatagtatcgacacactctggacgctggtcgtgtgatggactgttgccgccacacttgctgccttgacctgtgaatatccctgc
cgcttttatcaaacagcctcagtgtgtttgatcttgtgtgtacgcgcttttgcgagttgctagctgcttgtgctatttgcgaataccaccccca
gcatccccttccctcgtttcatatcgcttgcatcccaaccgcaacttatctacgctgtcctgctatccctcagcgctgctcctgctcctgctc
actgcccctcgcacagccttggtttgggctccgcctgtattctcctggtactgcaacctgtaaaccagcactgcaatgctgatgcacggg

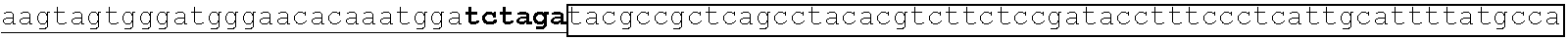

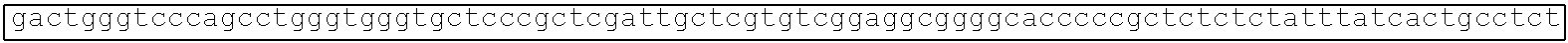

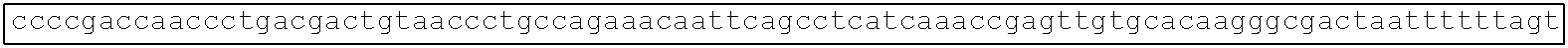

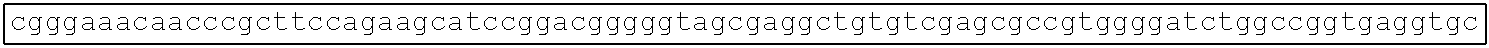

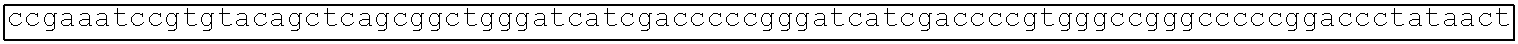

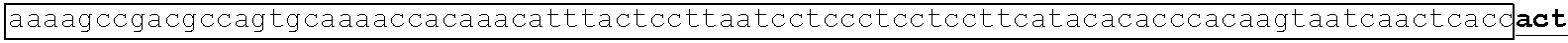

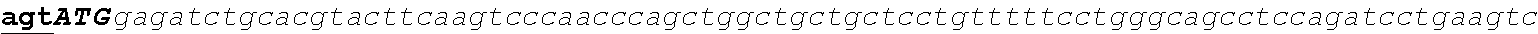

gacgttctccctcctgaagagcctgtacatctacttcctgcgccccggcaagaacctccgccgctacgggtcctgggccattatcac
cggcccgaccgacggcatcggcaaggcctttgcgttccagctggcccacaagggcctgaacctggtgctggtggcgcgcaaccc
ggacaagctgaaggacgtctccgacagcatcaggtccaagcatagcaacgtgcagatcaagacggtgatcatggactttagcg
gcgacgttgacgacggcgtccgccgcatcaaggagaccatcgaggggctggaggtgggcatcctgatcaacaatgccggcatg
tcctacccgtacgcgaagtactttcacgaggtcgacgaggagctcgtcaacggcctcatcaaaatcaacgtcgagggcacgacc
aaggtgacccaggccgtgctgccgggcatgctggagcgcaagcgcggcgccatcgtcaacatgggcagcggcgcggccgccc
tgatcccgtcgtaccccttctacagcgtgtatgccggcgcgaagacgtacgtggaccagttcacccggtgcctgcacgtcgagtac
aagaagagcggcattgacgtccagtgccaggtcccgctctacgtggccacgaagatgacgaagatccgccgcgcctccttcctg
gtcgcctcccccgagggctacgccaaggccgccctgcggttcgtggggtacgaggcccggtgcaccccctactggccgcacgcc
ctgatgggctacgtcgtctccgccctgccccagtccgtgttcgagtccttcaacatcaagcgctgcctgcagatccgcaagaaggg

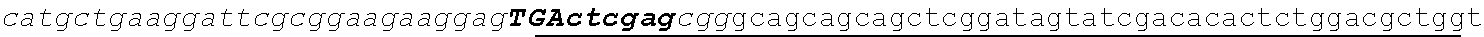

cgtgtgatggactgttgccgccacacttgctgccttgacctgtgaatatccctgccgcttttatcaaacagcctcagtgtgtttgatcttgtg
tgtacgcgcttttgcgagttgctagctgcttgtgctatttgcgaataccacccccagcatccccttccctcgtttcatatcgcttgcatccca
accgcaacttatctacgctgtcctgctatccctcagcgctgctcctgctcctgctcactgcccctcgcacagccttggtttgggctccgcc
tgtattctcctggtactgcaacctgtaaaccagcactgcaatgctgatgcacgggaagtagtgggatgggaacacaaatggagatatc

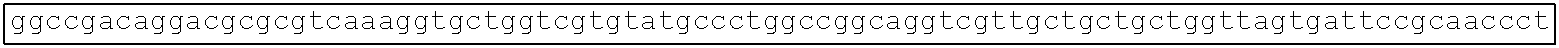

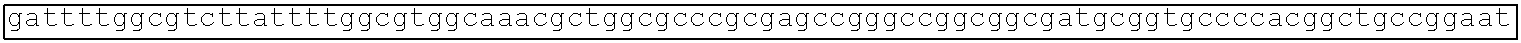

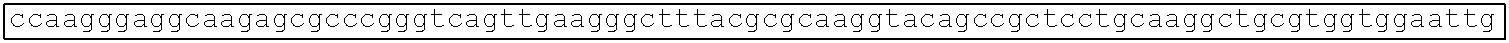

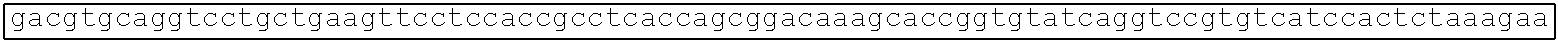

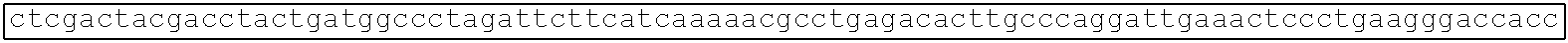

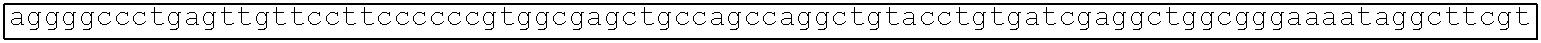

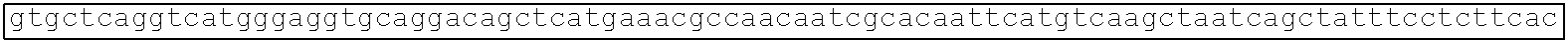

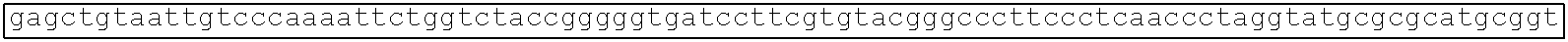

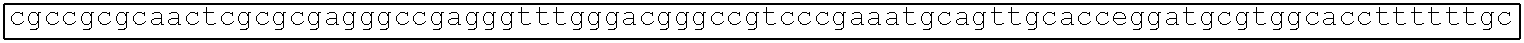

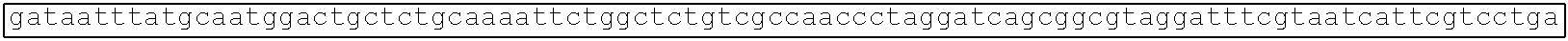

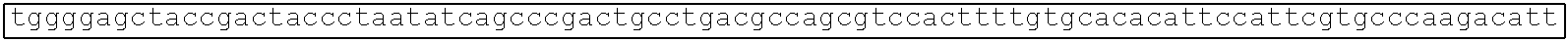

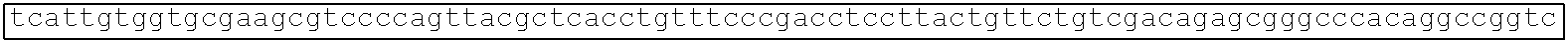

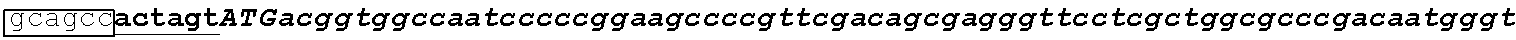

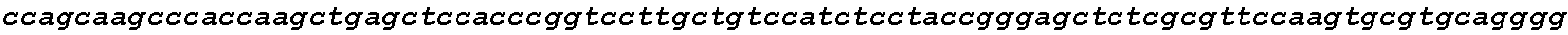

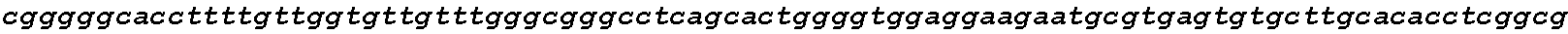

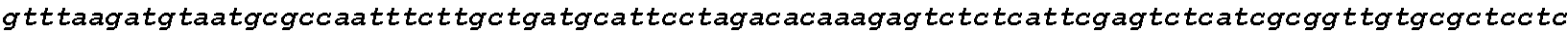

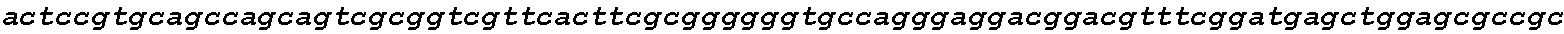

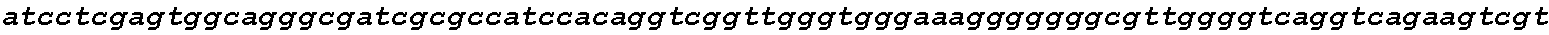

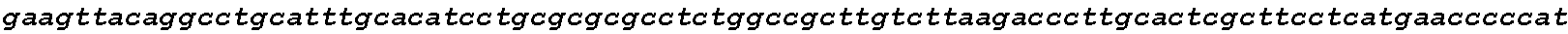

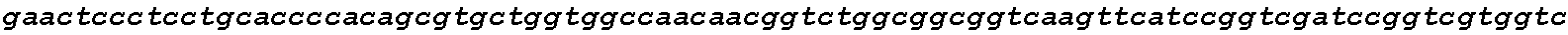

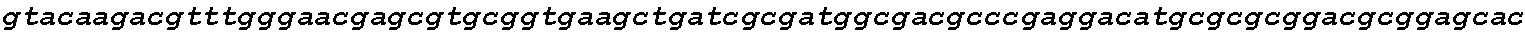

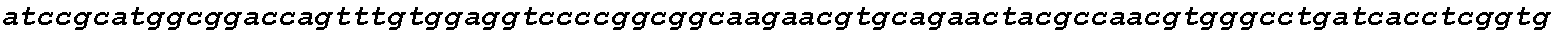

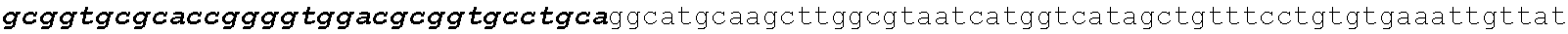

ccgctcacaattccacacaacatacgagccggaagcataaagtgtaaagcctggggtgcctaatgagtgagctaactcacattaat
SEQ ID NO: 150

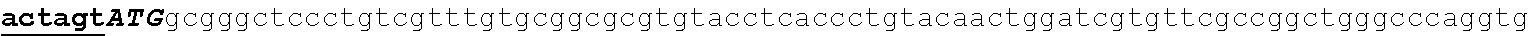

ctgtactttgccgtcaagacgctcaaggagtccggccacgagaacgtgtacgacgccgtggagaagcccctccagctggcgcaaac
cgccgcggtcctggagatcctccacggcctggtcggcctcgtcaggagcccggtctcggccaccctgccgcagatcgggagccgc
ctctttctgacctggggcattctgtattccttcccggaggtccagagccactttctggtgacctccctcgtgatcagctggtcgatcacgg
aaatcatccgctacagcttcttcggcctgaaggaggcgctgggcttcgcgcccagctggcacctgtggctccgctattcgagctttctg
gtgctctaccccaccggcatcacctccgaggtcggcctcatctacctggccctgccgcacatcaagacgtcggagatgtactccgtcc
gcatgcccaacaccagaactatccacgactattctacgccacgattctcgtcctcgcgatctacgtccccggacgccccacatgtacc

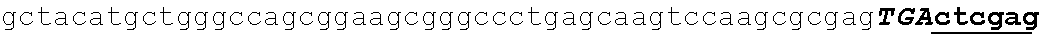

SEQ ID NO: 151
gattcatatcatcaaatttcgcatatgtttcacgagttgctcacaacatcggcaaatgcgttgttgttccctgttttacaccttgcc
agggcctggtcaaagcttgacagtttgaccaaattcaggtggcctcatctattcgcactgatagacattgcagatttggaagac
ccagccagtacattacatgcacagccatttgctcctgcaccatgaacttgccacttttgtgcgccggtcgggggtgatagctcgg
cagccgccgatcccaaaggtcccgcggcccaggggcacgagaccccccgacacgattaaatagccaaaatcagtcagaac
ggcacctccaccctacccgaatctgacaaggtcatcaaacgcgcgaaacaacggcgagggtgcgttcgggaagcgcgcgta
gttgacgcaagaagcctgggtcaggctggagggccgcgagaagatcgcttcctgccgagtdgcacccacgcctcgagcgca
ccgtccgcgaacaaccaaccccttttcgcgagccaggcattctttcaattgccaaggatgcacatgtgacacgtatagccattc
ggctttgtttgtgcctgcttgactcgcgccatttaattgttttgtgccggtgagccgggagtcggccactcgtaccgagccgcag
tcccggcgccagtcccccggcctctgatctgggtccggaagggttggtataggagcagtctcggctatctgaagcccgttacca
gacactttggccggctgctttccaggcagccgtgtactcttgcgcagtcggtacc
SEQ ID NO: 152

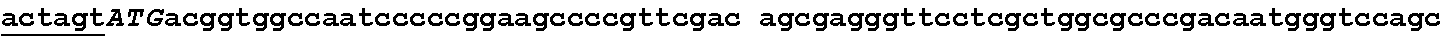

aagcccaccaagctgagctccacccggtccctgctgtccatctcctaccgggagctctcgcgttccaagtgcgtacaggggcg
agggcaccttttgttggtgttgtttgggcgggcctcggtactgggaggaggaggaatgcgtgcacacctctgcggttttagatgc
aatgcgacaagtgcctgctgatgcattttctagacatgaagcatctcgtattcgagtctcaacgcgggtgtgcgctcctcactcc
gtgcagccagcagtcgcggtcgttcacttcgcggggggtgccagggaggacggacgtttcggatgagctggagcgccgcatc
ctcgagtggcagggcgatcgcgccatccacaggtcggttgggtgggaaagggggagtaccggggtcaggtcagaagtcgtg
catttacaggcatgcatctgcacatcgtgcgcacgcgcacgtctttggccgcttgtctcaagactcttgcactcgtttcctcatgc
accataatcaattccctcccccctcgcaaactcacagcgtgctggtggccaacaacggtctggcggcggtcaagttcatccggt
cgatccggtcgtggtcgtacaagacgtttgggaacgagcgcgcggtgaagctgattgcgatggcgacgcccgagggcatgcg
cgcggacgcggagcacatccgcatggcggaccagtttgtggaggtccccggcggcaagaacgtgcagaactacgccaacgt
gggcctgatcacctcggtggcggtgcgcaccggggtggacgcggtgcctgcagg

1. An oleaginous eukaryotic microalgal cell that produces a cell oil, the cell optionally of the genus Prototheca, the cell comprising an ablation of one or more alleles of an endogenous polynucleotide encoding a lysophosphatidic acid acyltransferase (LPAAT).

2. The cell of claim 1, wherein the endogenous polynucleotide encoding the LPAAT has at least 80, 85, 90 or 95% sequence identity to SEQ ID NOs: 105 or 106.

3. The cell of claim 1, further comprising an exogenous gene encoding an active enzyme selected from the group consisting of
(a) a lysophosphatidylcholine acyltransferase (LPCAT);
(b) a phosphatidylcholine diacylglycerol cholinephosphotransferase (PDCT);
(c) CDP-choline:1,2-sn-diacylglycerol cholinephosphotransferase (DAG-CPT);
(d) a lysophosphatidic acid acyltransferase LPAAT; and
(e) a fatty acid elongase (FAE).

4. The cell of claim 3, wherein the exogenous gene encodes a lysophosphatidylcholine acyltransferase having at least 80, 85, 90 or 95% sequence identity to SEQ ID NOs: 98, 99, 100, 101, 102, or 108.

5-7. (canceled)

8. The cell of claim 3, wherein the exogenous gene encodes a fatty acid elongase having at least 80, 85, 90 or 95% sequence that encodes the amino acid of SEQ ID NO: 19, 20, 84 or 85.

9-31. (canceled)

32. An oleaginous eukaryotic microalgal cell that produces a cell oil, the cell optionally of the genus Prototheca, the cell comprising a first exogenous gene encoding an active enzyme of one of the following types:
(a) a lysophosphatidylcholine acyltransferase (LPCAT);
(b) a phosphatidylcholine diacylglycerol cholinephosphotransferase (PDCT); or
(c) CDP-choline:1,2-sn-diacylglycerol cholinephosphotransferase (DAG-CPT);
(d) an LPAAT;
(e) and optionally a second exogenous gene encoding
(f) a fatty acid elongase (FAE).

33. The cell of claim 32, wherein the cell comprises a fatty acid elongase enzyme having at least 80, 85, 90 or 95% sequence identity to SEQ ID NOs: 20, 84 or 85.

34. The cell of claim 32, wherein the first exogenous gene encodes a phosphatidylcholine diacylglycerol cholinephosphotransferase having at least 80, 85, 90 or 95% sequence identity to SEQ ID NO: 93.

35. The cell of claim 32, wherein the first exogenous gene encodes a lysophosphatidylcholine acyltransferase having at least 80, 85, 90 or 95% sequence identity to SEQ ID NOs: 98, 99, 100, 101, 102, or 108.

36. The cell of claim 32, wherein the first exogenous gene encodes an LPAAT having at least 80, 85, 90 or 95% sequence identity to SEQ ID NOs: 12, 29, 30, 32, 33, or 34.

37-53. (canceled)

54. An oleaginous eukaryotic microalgal cell that produces a cell oil, the cell optionally of the genus Prototheca, the cell comprising an exogenous polynucleotide that encodes an active ketoacyl-CoA reductase, hydroxyacyl-CoA dehydratase, or enoyl-CoA reductase.

55. The oleaginous eukaryotic microalgal cell of claim 54, wherein the exogenous polynucleotide has at least 80, 85, 90 or 95% sequence identity to SEQ ID NO: 144 and encodes an active ketoacyl-CoA reductase.

56. The oleaginous eukaryotic microalgal cell of claim 54, wherein the exogenous polynucleotide has at least 80, 85, 90 or 95% sequence identity to SEQ ID NO: 143 and encodes an active hydroxyacyl-CoA dehydratase.

57. The oleaginous eukaryotic microalgal cell of claim 54, wherein the exogenous polynucleotide has at least 80, 85, 90 or 95% sequence identity to the enoyl-CoA reductase encoding portion of SEQ ID NO: 142 and encodes an active enoyl-CoA reductase.

58. The oleaginous eukaryotic microalgal cell of claim 54, wherein the cell further comprises an exogenous nucleic acid encoding a lysophosphatidylcholine acyltransferase (LPCAT), a phosphatidylcholine diacylglycerol cholinephosphotransferase (PDCT), CDP-choline:1,2-sn-diacylglycerol cholinephosphotransferase (DAG-CPT), a lysophosphatidic acid acyltransferase (LPAAT) or a fatty acid elongase (FAE).

59. The oleaginous eukaryotic microalgal cell of claim 58, wherein the cell further comprises an exogenous nucleic acid encoding an enzyme selected from the group consisting of a sucrose invertase and an alpha galactosidase.

60. The oleaginous eukaryotic microalgal cell of claim 54, wherein the cell further comprises an exogenous nucleic acid that encodes a desaturase and/or a ketoacyl synthase.

61-64. (canceled)

65. An oil produced by an oleaginous eukaryotic microalgal cell, the cell optionally of the genus Prototheca, the cell comprising an exogenous polynucleotide that encodes an active ketoacyl-CoA reductase, hydroxyacyl-CoA dehydratase, or enoyl-CoA reductase.

66. The oil of claim 65, wherein the exogenous polynucleotide has at least 80, 85, 90 or 95% sequence identity to SEQ ID NO: 144 and encodes an active ketoacyl-CoA reductase.

67. The oil of claim 65, wherein the exogenous polynucleotide has at least 80, 85, 90 or 95% sequence identity to SEQ ID NO: 143 and encodes an active hydroxyacyl-CoA dehydratase.

68. The oil of claim 65, wherein the exogenous polynucleotide has at least 80, 85, 90 or 95% sequence identity to the enoyl-CoA reductase encoding portion of SEQ ID NO: 142 and encodes an active enoyl-CoA reductase.

69. The oil of claim 65, wherein the cell further comprises an exogenous nucleic acid encoding a lysophosphatidylcholine acyltransferase (LPCAT), a phosphatidylcholine diacylglycerol cholinephosphotransferase (PDCT), CDP-choline:1,2-sn-diacylglycerol cholinephosphotransferase (DAG-CPT), a lysophosphatidic acid acyltransferase (LPAAT) or a fatty acid elongase (FAE).

70. The oil of claim 69, wherein the cell further comprises and exogenous nucleic acid encoding an enzyme selected from the group consisting of a sucrose invertase and an alpha galactosidase.

71-110. (canceled)
